(19) United States
(12) Patent Application Publication
(10) Pub. No.: US 2003/0138432 A1
(43) Pub. Date: Jul. 24, 2003

(54) Selective cellular targeting: multifunctional delivery vehicles, multifunctional prodrugs, use as antineoplastic drugs

(75) Inventor: Arnold Glazier, Newton, MA (US)

Correspondence Address: N. Scott Pierce, Esq., HAMILTON, BROOK, SMITH & REYNOLDS, P.C., Two Militia Drive, Lexington, MA, 02421-4799, US

(73) Assignee: Drug Innovation & Design, Inc.

(21) Appl. No.: 09/738,625

(22) Filed: Dec. 15, 2000

Related U.S. Application Data
(63) Continuation of application No. 09/712,465, filed on Nov. 15, 2000, now abandoned.
(60) Provisional application No. 60/165,485, filed on Nov. 15, 1999.
(60) Provisional application No. 60/239,478, filed on Oct. 11, 2000.
(60) Provisional application No. 60/241,939, filed on Oct. 10, 2000.

(51) Int. Cl. A61K 39/395

(52) U.S. Cl. 424/178.1

(57) ABSTRACT

The present invention relates to the compositions, methods, and applications of a novel approach to selective cellular targeting. The purpose of this invention is to enable the selective delivery and/or selective activation of effector molecules to target cells for diagnostic or therapeutic purposes. The present invention relates to multi-functional prodrugs or targeting vehicles wherein each functionality is capable of enhancing targeting selectivity, affinity, intracellular transport, activation or detoxification. The present invention also relates to ultra-low dose, multiple target, multiple drug chemotherapy and targeted immunotherapy for cancer treatment.

### BACKGROUND OF THE INVENTION

Approximately 8 million Americans have a history of cancer. An estimated 500,000 people in the U.S. die from cancer yearly. The need for new and improved anti-cancer drugs is clear and compelling. The goal of cancer chemotherapy is to kill all malignant cells without undo toxicity to the patient. The fundamental technical obstacle to the development of safe and effective anti-cancer drugs is the problem of tumor selectivity. Cells become malignant by the abnormal regulation of normal cellular functions caused by changes in DNA. With few exceptions the quest for an enzyme or target which is absolutely selective for malignant cells has been elusive. Furthermore, it has become increasingly evident that an enormous number of gene defects that interfere with the regulation of cell growth and proliferation can cause cancer or reinforce the malignant state.

Hard learned lessons in pediatric oncology have defined the clinical requirements for the complete eradication of cancer. The administration of multiple drugs each capable of independently giving a 1-3 log reduction of tumor burden without the combined drug toxicity producing unacceptable side effects. The following reference relates to this subject matter: Frei, E. III., “Curative Cancer Chemotherapy,” Cancer Res, 45(12 Pt 1):6523-37 (1985), the contents of which are incorporated herein by reference in their entirety. Drug toxicity due to the low selectivity of anti-cancer drugs is the fundamental barrier to the routine cure of cancer. A compounding factor is the development of drug resistance. Current therapeutic regimens attempt to deal with the problem of drug resistance by the administration of multiple agents. However, the combined toxicity of multiple agents limits the effectiveness of this approach. Enormous efforts have been directed to the development of highly selective anti-cancer drugs. Monoclonal antibodies have been employed as targeting agents for the delivery of cytotoxic drugs to tumors. However, very few antigens, that are absolutely tumor specific, are available for tumor targeting. In addition monoclonal antibodies are large molecules, and often do not penetrate well into tumors. Proteins and oligopeptides have also been used as targeting agents. Small molecules described as targeting agents include: folate, sigma receptor binding agents and agmatine. A variety of approaches have also been described to target cells by prodrugs, which are activated by enzymes that are increased in tumor cells. Despite great efforts a general solution to the problem of selective cell targeting and selective destruction of cancer cells remains elusive. This is the subject of the present invention.

### SUMMARY OR THE INVENTION

The present invention relates to the compositions, methods, and applications of a novel approach to selective cellular targeting. The purpose of this invention is to enable the selective delivery and/or selective activation of effector molecules to target cells for diagnostic or therapeutic purposes. The present invention relates to multi-functional prodrugs or targeting vehicles wherein each functionality is capable of enhancing targeting selectivity, affinity, intracellular transport, activation or detoxification. The present invention also relates to ultra-low dose, multiple target, multiple drug chemotherapy and targeted immunotherapy for cancer treatment.

### BREIF DESCRIPTION OF THE DRAWINGS

No drawings

### DETAILED DESCRIPTION OF THE INVENTION

Definitions:

Analog

A compound or moiety possessing significant structural similarity as to possess substantially the same function.

At a Target Cell

A phrase used to refer to in, on, or in the microenvironment of a target cell.

Binding Affinity

Tightness of binding between a ligand receptor.

Bioreversibly Masked Group

A chemical group that is derivatized in a bioreversible manner. For example, an ester group can be a bioreversibly masked group for a hydroxy group. A bioreversible masking group is a chemical group that when bonded with a second group produces a bioreversibly masked group for said second group.

Bioreversible Protecting Group

A chemical group or trigger that can be modified in vivo and wherein said modification unmasks the group which is protected.

Chemically Modify

To change the chemical property of a molecule by making one or more new chemical bonds and/or by breaking one or more chemical bonds of the molecule.

Connectivity

The sites at which chemical structures or functional groups are attached together to give a single molecule. For example, various connectivity between groups A, B, C include structures such as A-B-C, B-A-C, or A-C-B. Connectivity can be direct such as by a covalent bond between an atom of A and B or indirect such as through a covalently bonded linker.

Derivative

A compound or moiety that has been further modified or functionalized from the corresponding compound or moiety.

Effector

An agent that exerts an activity and evokes a physical, chemical or biological response such as a pharmacologically beneficial response such as cytotoxicity, or a diagnostic effect.

Functional Cooperation between Components

If the effect produced by two or more components of a drug acting jointly or together is greater than the effect produced by the components acting individually or independently the components “functionally cooperate”.

Good Leaving Group

A chemical group that readily cleaves from the group to which it is attached. For example, a group that is easily displaced in a nucleophilic reaction, or which undergoes facile solvolysis in an SN1 type reaction.

Inert Substituents

A chemical substituent which does not interfere with functionality to a significant degree.

Linker

A chemical group that serves to attach targeting ligands, triggers and effectors or other chemical structures together.

Lower Alkyl Group

A hydrocarbon containing about 10 or less carbon atoms which can be linear or cyclic and which can bear substituents.

Masked Group

A chemical group that is hidden or blocked,or derivatized until unmasked.

Microenvironment of the Target

The volume of space around a target cell within which a drug is able to evoke its intended pharmacological activity upon the target. Alternatively, the volume encompassed by a sphere centered on a tumor cell with a radius of between about 10 to about 500 microns.

Multifactorial

A function of multiple factors or variables.

Multivalent Binding

Binding at multiple targeting ligand- target receptor sites.

Non-selective Targeting Ligand

A chemical structure that binds to a receptor or physically associates with biomolecules that are ubiquitous or not enriched on the target compared to non-target.

Non-target

A cell, cells, tissue, or tissue type to which it is not desired to direct effector activity, such as normal cells, bone marrow stem cells, or normal liver.

Over-expressed

present at increased amounts.

Pharmacological activity

A beneficial physical, chemical or biological response that is evoked by a drug or effector agent such as a cytotoxicity or stimulation of the immune system or a diagnostic effect.

Target

A cell, cells, tissue, or tissue type to which it is desired to direct effector activity such as tumor cells, or autoimmune lymphocytes.

Targeting Agent

A chemical structure or group of chemical structures composed of targeting ligand(s) and/or trigger(s) that confer a degree of specificity towards a target.

Targeting Ligand

A chemical structure, which binds with a degree of specificity to a targeting receptor that is enriched at a target cell compared to at a non-target cell.

Targeting Property

Any characteristic, feature, or factor, such as a targeting receptor, a triggering agent, an enzyme, or a chemical or biochemical factor that can be used to distinguish between target and non-target.

Targeting Receptor

A chemical structure at the target that binds with a useful degree of specificity to a targeting ligand that is present in increased amounts in a target compared to a non-target but not necessarily all non-targets.

Targeting Selectivity

The ability to evoke a greater effector activity at target compared to non-target.

Target Molecules

Biomolecules that are either target receptors or triggering agents such as a protein that binds a targeting ligand or an enzyme at the target cell which can activate a trigger and which are increased at a target compared to a non-target but not necessarily all non-targets.

Trigger

A chemical group which can undergo in vivo chemical modification either spontaneously or by a triggering agent with the modification leading to trigger activation that modulates the pharmacological activity of the drug. A trigger can be considered as a chemical switch that upon activation gives a consistent and predictable output such as unmasking a chemical group, or detoxifying the drug, or toxifying the drug, or liberating an effector agent.

Trigger Activation

The process of chemical modification that causes a trigger to modulate the pharmacological activity of the drug.

Triggering Factor

An enzyme, biomolecule or other agent which is able to activate a trigger, also referred to as a “triggering agent”.

Tumor Component

is a biomolecule which is present in tumor cells, on tumor cells, in the microenvironment of tumor cells, on tumor stromal cells or present in tumor bulk.

Tumor-selective Target Receptor

A target receptor that is present in increased amounts on tumor cells or in the microenvironment of tumor cells compared to that of normal cells but not necessarily all types of normal cells.

Tumor-selective Triggering Agent

A triggering agent or triggering factor that is present in increased amounts on tumor cells, in tumor cells, or in the microenvironment of tumor cells compared to that of normal cells but not necessarilly all types of normal cells.

Vital Normal Cells

Cells that if destroyed would produce unacceptable clinical toxicity to a patient such as bone marrow stem cells, liver cells and cardiac cells.

In order to eradicate cancer it is necessary to administer sufficient drugs to kill the last cancer cell without prohibitive toxicity to the patient. The poor selectivity and high toxicity of current anti-cancer drugs is the major road-block to routinely achieving this goal. What is needed is a technology that can allow the safe use of multiple drugs directed against multiple properties of the tumor without multiple toxicity. This invention relates to an integrated description of technologies directed towards this goal.

There are two fundamental problems in anti-cancer drug design and therapy:

1.) Absolute enzymatic differences between normal and malignant cells are with rare exceptions elusive.

2.) Tumors are heterogenous and can develop resistance to any drug.

In order for any type of therapy to selectively kill cancer cells the therapy must be directed to differences between normal cells and cancer cells.

There are two types of differences:

1.) Specific differences that are the causative lesions of cancer.

2.) Nonspecific differences that are secondary consequences of the causative lesions of cancer. These are the abnormal patterns of normal protein expresson that define the malignant phenotype.

It has become increasingly evident that an enormous range of DNA mutations, that disrupt critical regulatory pathways that control cell growth, can cause cancer and reinforce the malignant state in cancerous cells. Although the DNA mutations are specific to the cancer cells, targeting the mutations or the defective proteins that result from the DNA mutations may not be practical. It is likely that out of the estimated 140,000 genes in the human genome hundreds or perhaps thousands are capable of causing cancer. It is infeasible to prepare drugs that target each of these primary causes of cancer. In addition, many DNA mutations are known that induce malignant transformation by the loss of key regulatory proteins. In these cases the only way to distinguish the normal from malignant cells is by the secondary consequences that result from the absence of the regulatory protein. These consequences are the abnormal patterns of normal protein expression that define the malignant state. Although individually the proteins are normal and not unique to malignant cells the patterns of protein expression are highly specific to cancer. DNA mutations are the spark, but abnormal patterns of normal protein expression are the explosion and fire that is cancer. Anti-cancer drugs must be able to recognize the abnormal patterns of normal protein expression that define the malignant state. This is the purpose of the present invention.

These considerations highlight an important principle central to the problem of anti-cancer therapy. Anti-cancer therapies should be multifactorial unless directed against a causative lesion of cancer.

The hallmark of malignancy is uncontrolled cell proliferation and tissue invasion. The biochemical manifestation of these processes provides the basis for understanding and defining optimal tumor targeting. Neither the processes of cell replication nor the enzymology of tissue invasion (remodeling) are by themselves uniquely diagnostic of malignancy. But jointly, these processes likely provide highly selective criteria to define effective targeting for the treatment of malignancy. The current class of multifunctional anti-cancer drugs provides the opportunity to have anti-cancer agents that are targeted simultaneously and jointly to both the proliferative and the invasive character of malignant cells.

In order to achieve tumor selectivity it is necessary to make drugs that can identify cancer cells. It is possible for a pathologist to distinguish malignant from normal cells in biopsies because the diagnostic criteria are multiple. Multiple factors such as cell size, shape, organization, location, and histochemistry allow differentiation between normal and malignant cells. In contrast, present anti-cancer drugs are essentially monofactorial directed against one property of malignancy such as cellular replication, invasiveness, or a tumor antigen. These individual properties are not unique to cancer cells and severely limit the selectivity of present anti-cancer drugs. The hallmark of malignancy is uncontrolled proliferation and invasiveness. The biochemistry of either alone is nonspecific. Jointly these properties characterize malignancy.

Although a single property or characteristic is not unique to malignant cells the pattern of expression of multiple such properties can provide almost absolute tumor specificity. Exquisite antitumor selectivity can be obtained by multifactorial drugs that target cells only if the cells jointly express multiple properties associated with the malignant phenotype. The present invention relates to technologies that can enable multifactorially targeted toxicity that is a consequence of multifactorial target recognition, effector action, or both. The present invention relates to a class of multifunctional, multifactorial drugs with pattern recognition capabilities. The present technology also relates to compositions and methods by which selective multifactorial toxicity can be achieved by delivering multiple monofactorially targeted effector molecules. The invention also relates to key patterns of protein expression useful for selectively targeting cancer.

The present invention is a technology, which can allow the selective targeting of tumors with ultra-low doses of multiple drugs directed against multiple tumor targets. The high selectivity and high affinity of the drugs for tumor cells can enable the total dose of chemotherapy to be reduced thousands of times below current levels. The severe side effects currently associated with chemotherapy are not expected with ultra-low dose multiple drug therapy. Most importantly, the simultaneous use of multiple drugs directed against multiple tumor targets can potentially eliminate the problem of tumor resistance. The probability that a tumor could simultaneously develop resistance to ten independent drugs each capable of giving a 2 log reduction in tumor burden is essentially zero.

A second major application of the technology described in this patent is targeted immunotherapy in which an intense immune response directed against non-tumor antigens is specifically targeted to tumors to elicit tumor rejection. In addition, technology is described that can allow the targeted formation of neotumor antigens.

The present invention relates to the compositions, methods, and applications of a novel approach to selective cellular targeting. The purpose of this invention is to enable the selective delivery and/or selective activation of effector molecules to target cells for diagnostic or therapeutic purposes. The present invention relates to multi-functional prodrugs or targeting vehicles wherein each functionality is capable of enhancing targeting selectivity, affinity, intracellular transport or activation. The present invention can be used to selectively target cells for diagnostic or therapeutic purposes. The principle applications are in the field of anti-cancer therapy. However, the applications are not limited to the delivery of antineoplastic drugs and can be employed in other applications where selective drug targeting is beneficial such as in the delivery of immunosuppressants.

Most current anti-cancer drugs are nonspecific or have low selectivity for tumor cells versus normal cells. The present invention seeks to address this problem by exploiting more than one property of tumor cells to define drug selectivity through the use of multi-functional delivery vehicles or prodrugs. Multifunctionality is also exploited to prevent the emergence of tumor drug resistance, and to selectively detoxify the drug in vital normal cells and to selectively toxify the drug in tumor cells.

Polymeric drugs and dendritic type drugs are well known, but do not provide an adequate solution to the seletive targeting and destruction of tumor cells even when connected to a targeting group such as a monoclonal antibody. The fundamental problem remains targeting specificity. The following references relates to this subject mafter: WO 99/53951, Oct. 28, 1999, Martinez, et al., “Terminally-Branched Polymeric Linkers and Polymeric Conjugates Containing the Same”; U.S. Pat. No. 5,783,178, Jul. 21, 1998 Kabanov, et al., “Polymer Linked Biological Agents.”; Schacht E. H., et al., “Macromolecular Carriers for Drug Targeting,” Wermuth C. G. (ed), The Practice of Medicinal Chemistry, Academic Press Limited, 1996, pp.717-736, the contents of which are incorporated herein by reference in their entirety.

The present invention also encompasses (embodiment ET1) A compound ET wherein E is comprised of one or more effector agents having pharmacological activity designated as “PA” and T is comprised of a targeting agent comprised of two or more groups each of which functions to specifically enhance the targeting selectivity by either increasing the pharmacological activity PA at targeted cells and/or decreasing the pharmacological activity PA at non-target cells;

and provided that at least one component of T is comprised of a group designated as a “selective targeting ligand” that binds specifically to a site designated as a “selective targeting receptor” on the target;

and wherein if a second selective targeting ligand is present in T then the first and second targeting ligands are able to bind simultaneously to two targeting receptor molecules;

and provided that T is not an antibody, or an analog or component of an antibody, or a complex of antibodies, or a bispecific antibody, or an analog of a bispecific antibody, or a natural protein, or a complex of natural proteins, or a protein, or a naturally occurring polymer, or a radiolabelled dimer, or a polymer to which is attached at multiple sites one or more pharmacologically active compounds that mediate the same pharmacological activity PA.

The present invention also relates to the method of selectively targeting cells by the administration of said compound.

The present invention addresses the following critical aspects of antitumor drug function:

1.) Targeting specificity or the ability to localize the drug selectively to tumor cells.

2.) Transport of the targeted drug into the tumor cells.

3.) Triggering or activation to liberate the cytotoxic moiety at or in the tumor cell.

4.) Detoxification: the ability to selectively detoxify the drug to protect vital normal cells.

5.) Prevention of drug resistance.

Mechanism of Action

The mechanisms of actions and scientific basis of the present invention are described beginning on page 122.

The present invention encompasses a method (embodiment M1) to evoke a greater effector activity referred to as the pharmacological activity “PA”; at target cells compared to non-target cells;

wherein at the target cells there are present “m” different types of target molecules designated as (p1 . . . pm); at least one of which is present at increased amounts compared to at a non-target cell, and wherein the type of the targeting molecule which is increased on the target cells compared to a non-target cell can be different for a different non-target cell;

and wherein at non-target cells there can be present the same types of target molecules (p1 . . . pm);

wherein target molecules are biomolecules that are either target receptors or triggering agents;

wherein a target receptor is a chemical structure at the target cells that binds with a useful degree of specificity to a targeting ligand wherein said target receptor is present in increased amounts at the target cells compared to at some non-target cells;

and wherein a “triggering agent” is an enzyme, or biomolecule or other agent which is able to activate a trigger and which is increased at a target compared to at some non-target cells;

and wherein the method is comprised of contacting the cell or cell populations with one or more compounds designated as (C1 . . . Cn), wherein at least one of the compounds has the structure E₁T₁; wherein E₁ is comprised of x effector agents that evoke the pharmacological activity PA, and T₁ is comprised of the y different targeting ligands, and z different triggers, which increase the pharmacological activity PA at targeted cells and/or decrease the pharmacological activity PA at non-target cells;

and wherein a targeting ligand comprises a chemical structure, which binds with a degree of specificity to a targeting receptor that is enriched at a target cell compared to at a non-target cell;

and wherein a trigger is a chemical group which can undergo in vivo chemical modification either spontaneously or by a triggering agent with the modification leading to trigger activation that modulates the pharmacological activity of the drug;

and wherein the number m is 2, 3, 4, 5, 6, 7, 8, 9, 10, 11, 12, 13, 14, 15, 16, 17, 18, 19, or 20, or about 20;

and wherein the number x is 1, 2, 3, 4, 5 or about 5;

and wherein the number y is 1, 2, 3, 4, 5, 6, 7, 8, 9, or 10 or about 10;

and wherein the number z is 0, 1, 2, 3, 4, 5, 6, 7, 8, 9, or 10 or about 10;

and n is 1, 2, 3, 4, 5, 6, 7, 8, 9, or 10 or about 10;

and wherein if n equals one then the sum of y and z is equal to or greater than m;

and wherein if n>1 the selectivity of the evoked response in targeted cells is not due solely to internalization and functional cooperation of the different effector groups inside the cells.

The present invention also encompasses (embodiment ET2) a multifunctional drug delivery vehicle which comprises a compound ET wherein E is comprised of one or more effector agents designated as E1 . . . En wherein n=1, 2, 3, 4, or 5 or about 5 and wherein these effector agents have pharmacological activity referred to as “PA”; and wherein T comprises a targeting agent which comprises: targeting ligands; or targeting ligands and triggers; and wherein T increases the pharmacological activity PA to a target cell compared to a non-target cell;

and wherein a targeting ligand is a group that binds selectively to a structure associated with the target referred to as a “targeting receptor”;

and wherein a trigger is a group that upon in vivo modification by biomolecules referred to as “triggering agents” becomes activated and modulates the activity of ET;

and wherein at the target cells there are present “m” different types of target molecules designated as (p1 . . . pm); at least one of which is present at increased amounts compared to at a non-target cell, and wherein the type of the targeting molecule which is increased on the target cells compared to a non-target cell can be different for a different non-target cell;

and wherein at non-target cells there can be present the same types of target molecules (p1 . . . pm);

wherein the number m is 2, 3, 4, 5, 6, 7, 8, 9, 10, 11, 12, 13, 14, 15, 16, 17, 18, 19, or 20.

In a preferred embodiment the number m is 2, 3, 4, 5, or about 6.

The present invention also encompasses (embodiment ET3) a compound ET in which E is comprised of one or more effector agents having pharmacological activity designated as “PA” and wherein T comprises:

a) A group referred to as a “targeting ligand” which selectively binds to a target receptor on the surface of the target cell or in the microenvironment of the target cell; and

b) One or more of the following:

I. A targeting ligand which selectively binds to a target receptor on the surface of the target cell or in the microenvironment of the target cell;

II. A group, referred to as a “masked intracellular transport ligand” which can be modified in vivo to give a group referred to as an “intracellular transport ligand” which binds to a target cell receptor that actively transports bound ligands into the target cell;

III. A group referred to as a “trigger” that can be modified in vivo, wherein in vivo modification activates the trigger and modulates the pharmacological activity PA; and

IV. A group referred to as an “intracellular trapping ligand”, which binds to one or more intracellular receptors or a group referred to as a “masked intracellular trapping ligand” which can be modified in vivo to give an “intracellular trapping ligand”;

and wherein if a second targeting ligand is present in T then the first and second targeting ligands are able to bind simultaneously to two targeting receptor molecules;

and wherein if T is comprised solely of a targeting ligand a trigger and in vivo modification of the trigger increases the pharmacological activity PA then the in vivo modification which activates the trigger is caused by an enzyme or enzymatic activity that is increased at target cells or decreased at non-target cells;

and wherein if T is comprised solely of a targeting ligand a trigger and in vivo modification of the trigger decreases the pharmacological activity PA then the in vivo modification which activates the trigger is caused by an enzyme or enzymatic activity that is decreased at target cells or increased at non-target cells;

and provided that T is not an antibody, or an analog or component of an antibody, or a complex of antibodies, or a bispecific antibody, or an analog of a bispecific antibody, or a natural protein, or a complex of natural proteins, or a protein, or a naturally occurring polymer, or a radiolabelled dimer, or a polymer to which is attached at multiple sites one or more pharmacologically active compounds that evoke the same pharmacological activity PA.

A preferred embodiment, of all the prior embodiments of ET, comprises ET wherein ET evokes a greater pharmacological activity PA at the target cell compared to a non-target cell and wherein this target cell selectivity is due to functional cooperation between the components of ET and not due to any single component of ET acting alone.

A preferred embodiment comprises ET wherein ET is comprised of a compound in which the targeting ligand selectively binds to a target receptor on the surface of the target cell or in the microenvironment of the target cell wherein the concentration of the target receptor is greater on the surface of the target cell or in the microenvironment of the target cell than on the surface or in the microenvironment of non-target cells.

A preferred embodiment of the present invention (embodiment ET4) comprises ET wherein ET is comprised of a compound with two or more targeting ligands wherein at least one of the targeting ligands binds to a target receptor on the surface of the target cell or in the microenvironment of the target cell wherein the target has an increased amount of that target receptor compared to a non-target cell that binds to a second targeting ligand of the compound. Generally, the increased amount is greater than about two times or greater than about 5 times, or greater than about 10 times. A preferred embodiment is comprised of ET in which at least one of the targeting ligands binds to a receptor that is absent or essentially absent from a non-targeted cell.

Methods for detecting increased amounts of receptors are well known to one skilled in the arts and include immunohistochemistry, radioimmunoassays, enzymatic assays, and a variety of nucleic acid hybridization techniques.

A preferred embodiment (embodiment ET5) comprises ET wherein ET is comprised of a compound with two or more targeting ligands that binds to a target cell with an affinity that is greater than a non-target cell presenting a target receptor(s) that bind to the targeting ligands of said compound. In preferred embodiments the above mentioned binding affinity to the target cell is at least about 2-5 times greater, or at least about 5-10 times greater, or at least about 10-50 times greater, or at least about 50-500 times greater, or at least about 500-5000 times greater, or at least about 5000-50,000 times greater, or at least about 50,000-1,000,000 times greater or more then 1 million times greater than to the non-target cell.

A preferred embodiment (embodiment ET6) comprises ET wherein ET is comprised of a drug with binding affinity to target cells that is approximately the same as to a population of non-target cells however said population of non-target cells have decreased sensitivity to the effects of the effector agent because said normal cells have decreased levels of an intracellular trapping receptor, or decreased sensitivity to the effector agent, or decreased levels of a specific protein necessary for neoantigen formation, or decreased levels of an enzyme that activates a trigger that increases the toxicity of ET, or increased levels of an enzyme that activates a trigger that decreases the toxicity of ET, or by virtue of said normal cells being located in the body at a site such as the brain where the drug ET cannot penetrate to a significant degree.

A preferred embodiment of ET is comprised of a compound in which the intracellular trapping ligand selectively binds to one or more intracellular receptors wherein the concentration of the intracellular receptors is greater in target cells than in non-target cells.

A preferred embodiment of ET is comprised of a compound with a trigger that increases the pharmacological activity PA upon in vivo modification and wherein the in vivo modification that activates the trigger is caused by an enzyme or enzymatic activity that is increased at target cells or decreased at non-target cells.

A preferred embodiment of ET is comprised of a compound with a trigger that decreases the pharmacological activity PA upon in vivo modification and wherein the in vivo modification that activates the trigger is caused by an enzyme or enzymatic activity that is decreased at target cells or increased at non-target cells.

A preferred embodiment of ET is comprised of a compound in which the intracellular transport ligand binds to a molecule referred to as a “transporter molecule” to form a complex and wherein this complex binds to a target cell receptor that actively transports bound ligands into the target cell.

A preferred embodiment of ET is comprised of a compound in which the concentration of transporter molecules is increased at the surface of target cells compared to non-target cells.

A preferred embodiment of ET is comprised of a compound with two targeting ligands that selectively bind to target receptors on the surface of the target cell or in the microenvironment of the target cell wherein the concentration of the target receptors is greater on the surface of the target cell or in the microenvironment of the target cell than on the surface or in the microenvironment of non-target cells. In a preferred embodiment these targeting ligands are the same. In another preferred embodiment these targeting ligands are different and bind to different types of targeting receptors.

A preferred embodiment of ET is comprised of a compound with three targeting ligands that selectively bind to target receptors on the surface of the target cell or in the microenvironment of the target cell wherein the concentration of the target receptors is greater on the surface of the target cell or in the microenvironment of the target cell than on the surface or in the microenvironment of non-target cells. In a preferred embodiment these targeting ligands are the same. In another preferred embodiment these targeting ligands are different and bind to different types of targeting receptors.

A preferred embodiment of ET is comprised of a compound with four targeting ligands that selectively bind to target receptors on the surface of the target cell or in the microenvironment of the target cell wherein the concentration of the target receptors is greater on the surface of the target cell or in the microenvironment of the target cell than on the surface or in the microenvironment of non-target cells. In a preferred embodiment these targeting ligands are the same. In another preferred embodiment these targeting ligands are different and bind to different types of targeting receptors.

Another preferred embodiment of ET is comprised of a compound with two or more targeting ligands wherein at least one of the targeting ligands binds to a target receptor on the surface of the target cell or in the microenvironment of the target cell wherein the target has an increased amount of that target receptor compared to a non-target cell that binds to a second targeting ligand of the compound. A preferred embodiment of this embodiment comprises a compound with two different targeting ligands that bind to two different targeting receptors. Another preferred embodiment of this embodiment comprises a compound with three different targeting ligands that bind to three different targeting receptors. Another preferred embodiment of this embodiment comprises a compound with four different targeting ligands that bind to four different targeting receptors.

A preferred embodiment (embodiment ET7) of ET is comprised of the following groups:

I. N1 targeting ligands, which can differ;

II. N2 masked intracellular transport ligands which can differ;

III. N3 triggers, which can differ, designated “detoxification triggers” wherein activation of the trigger decreases the pharmacological activity PA;

IV. N4 effector agents which can differ;

V. N5 triggers which can differ, wherein activation of the trigger increases the pharmacological activity PA;

VI. N6 intracellular trapping ligands or masked intracellular trapping ligands, which can differ;

and wherein:

N1=1, 2, 3, 4, 5, 6, 7, 8, 9, 10, or about 10;

N2=0, 1, 2, 3, 4, or about 4;

N3=0, 1, 2, 3, 4, 5, or about 5;

N4=1, 2, 3, 4, 5, or about 5;

N5=0, 1, 2, 3, 4, 5, 6, 7, 8, 9, 10, or about 10; and

N6=0, 1, 2, 3, 4, 5, 6, 7, 8, 9, 10, or about 10;

and wherein the components are covalently coupled directly or by one or more linkers, and wherein the connectivity between groups can vary provided that the functionality of the different components remains intact and wherein the function of ligands is to bind to their respective receptors; the function of the triggers is to be activated and modulate drug activity, and the function of the effector agent is to evoke the pharmacological activity PA;

and wherein the linker lengths can be 1, 2, 3, 4, 5, 6, 7, 8, 9, 10, 11, 12, 13, 14, 15, 16, 17, 18, 19, . . . 300 bond lengths or about 300 bond lengths; wherein the ( . . . ) are meant to represent the continuation of the sequence of numbers up to 300.

The connectivity is not critical because the target molecules that the groups interact with are not rigidly fixed in space.

Detailed descriptions of each of the components of ET are given in later sections A preferred embodiment (embodiment ET8) comprises ET with

N1=1, 2, 3, or 4;

N2=0, 1, or 2;

N3=0, 1, or 2;

N4=1, 2, or 3;

N5=0, 1, 2, or 3;

N6=1, 2,or 3;

Additional preferred embodiments of ET (embodiment ET8.X wherein X=# in the list below) are listed on each line below wherein:

1) N1=1, N2=0, N3=1, N4=1, N5=0, and N6=0

2) N1=1, N2=0, N3=0, N4=2, N5=0, and N6=0

3) N1=1, N2=0, N3=0, N4=3, N5=0, and N6=0

4) N1=1, N2=0, N3=0, N4=1, N5=1, and N6=0

5) N1=1, N2=0, N3=0, N4=1, N5=2, and N6=0

6) N1=1, N2=0, N3=0, N4=1, N5=3, and N6=0

7) N1=1, N2=0, N3=0, N4=1, N5=0, and N6=1

8) N1=1, N2=0, N3=1, N4=2, N5=0, and N6=0

9) N1=1, N2=0, N3=1, N4=3, N5=0, and N6=0

10) N1=1, N2=0, N3=1, N4=1, N5=1, and N6=0

11) N1=1, N2=0, N3=1, N4=1, N5=2, and N6=0

12) N1=1, N2=0, N3=1, N4=1, N5=3, and N6=0

13) N1=1, N2=0, N3=1, N4=1, N5=0, and N6=1

14) N1=1, N2=0, N3=1, N4=2, N5=1, and N6=0

15) N1=1, N2=0, N3=1, N4=2, N5=1, and N6=1

16) N1=1, N2=0, N3=1, N4=2, N5=2, and N6=0

17) N1=1, N2=0, N3=1, N4=2, N5=2, and N6=1

18) N1=1, N2=0, N3=1, N4=2, N5=3, and N6=0

19) N1=1, N2=0, N3=1, N4=2, N5=3, and N6=1

20) N1=1, N2=0, N3=1, N4=2, N5=0, and N6=1

21) N1=1, N2=0, N3=1, N4=3, N5=1, and N6=0

22) N1=1, N2=0, N3=1, N4=3, N5=1, and N6=1

23) N1=1, N2=0, N3=1, N4=3, N5=2, and N6=0

24) N1=1, N2=0, N3=1, N4=3, N5=2, and N6=1

25) N1=1, N2=0, N3=1, N4=3, N5=3, and N6=0

26) N1=1, N2=0, N3=1, N4=3, N5=3, and N6=1

27) N1=1, N2=0, N3=1, N4=3, N5=0, and N6=1

28) N1=1, N2=0, N3=1, N4=1, N5=1, and N6=1

29) N1=1, N2=0, N3=1, N4=1, N5=2, and N6=1

30) N1=1, N2=0, N3=1, N4=1, N5=3, and N6=1

31) N1=1, N2=1, N3=0, N4=1, N5=0, and N6=0

32) N1=1, N2=1, N3=0, N4=2, N5=0, and N6=0

33) N1=1, N2=1, N3=0, N4=3, N5=0, and N6=0

34) N1=1, N2=1, N3=0, N4=1, N5=1, and N6=0

35) N1=1, N2=1, N3=0, N4=1, N5=2, and N6=0

36) N1=1, N2=1, N3=0, N4=1, N5=3, and N6=0

37) N1=1, N2=1, N3=0, N4=1, N5=0, and N6=1

38) N1=1, N2=1, N3=0, N4=2, N5=1, and N6=0

39) N1=1, N2=1, N3=0, N4=2, N5=1, and N6=1

40) N1=1, N2=1, N3=0, N4=2, N5=2, and N6=0

41) N1=1, N2=1, N3=0, N4=2, N5=2, and N6=1

42) N1=1, N2=1, N3=0, N4=2, N5=3, and N6=0

43) N1=1, N2=1, N3=0, N4=2, N5=3, and N6=1

44) N1=1, N2=1, N3=0, N4=2, N5=0, and N6=1

45) N1=1, N2=1, N3=0, N4=3, N5=1, and N6=0

46) N1=1, N2=1, N3=0, N4=3, N5=1, and N6=1

47) N1=1, N2=1, N3=0, N4=3, N5=2, and N6=0

48) N1=1, N2=1, N3=0, N4=3, N5=2, and N6=1

49) N1=1, N2=1, N3=0, N4=3, N5=3, and N6=0

50) N1=1, N2=1, N3=0, N4=3, N5=3, and N6=1

51) N1=1, N2=1, N3=0, N4=3, N5=0, and N6=1

52) N1=1, N2=1, N3=0, N4=1, N5=1, and N6=1

53) N1=1, N2=1, N3=0, N4=1, N5=2, and N6=1

54) N1=1, N2=1, N3=0, N4=1, N5=3, and N6=1

55) N1=1, N2=1, N3=1, N4=1, N5=0, and N6=0

56) N1=1, N2=1, N3=1, N4=2, N5=0, and N6=0

57) N1=1, N2=1, N3=1, N4=3, N5=0, and N6=0

58) N1=1, N2=1, N3=1, N4=1, N5=1, and N6=0

59) N1=1, N2=1, N3=1, N4=1, N5=2, and N6=0

60) N1=1, N2=1, N3=1, N4=1, N5=3, and N6=0

61) N1=1, N2=1, N3=1, N4=1, N5=0, and N6=1

62) N1=1, N2=1, N3=1, N4=2, N5=1, and N6=0

63) N1=1, N2=1, N3=1, N4=2, N5=1, and N6=1

64) N1=1, N2=1, N3=1, N4=2, N5=2, and N6=0

65) N1=1, N2=1, N3=1, N4=2, N5=2, and N6=1

66) N1=1, N2=1, N3=1, N4=2, N5=3, and N6=0

67) N1=1, N2=1, N3=1, N4=2, N5=3, and N6=1

68) N1=1, N2=1, N3=1, N4=2, N5=0, and N6=1

69) N1=1, N2=1, N3=1, N4=3, N5=1, and N6=0

70) N1=1, N2=1, N3=1, N4=3, N5=1, and N6=1

71) N1=1, N2=1, N3=1, N4=3, N5=2, and N6=0

72) N1=1, N2=1, N3=1, N4=3, N5=2, and N6=1

73) N1=1, N2=1, N3=1, N4=3, N5=3, and N6=0

74) N1=1, N2=1, N3=1, N4=3, N5=3, and N6=1

75) N1=1, N2=1, N3=1, N4=3, N5=0, and N6=1

76) N1=1, N2=1, N3=1, N4=1, N5=1, and N6=1

77) N1=1, N2=1, N3=1, N4=1, N5=2, and N6=1

78) N1=1, N2=1, N3=1, N4=1, N5=3, and N6=1

79) N1=1, N2=0, N3=0, N4=2, N5=1, and N6=0

80) N1=1, N2=0, N3=0, N4=2, N5=2, and N6=0

81) N1=1, N2=0, N3=0, N4=2, N5=3, and N6=0

82) N1=1, N2=0, N3=0, N4=2, N5=0, and N6=1

83) N1=1, N2=0, N3=0, N4=3, N5=1, and N6=0

84) N1=1, N2=0, N3=0, N4=3, N5=2, and N6=0

85) N1=1, N2=0, N3=0, N4=3, N5=3, and N6=0

86) N1=1, N2=0, N3=0, N4=3, N5=0, and N6=1

87) N1=1, N2=0, N3=0, N4=1, N5=2, and N6=1

88) N1=1, N2=0, N3=0, N4=1, N5=3, and N6=1

89) N1=1, N2=0, N3=0, N4=2, N5=1, and N6=1

90) N1=1, N2=0, N3=0, N4=2, N5=2, and N6=1

91) N1=1, N2=0, N3=0, N4=2, N5=3, and N6=1

92) N1=1, N2=0, N3=0, N4=3, N5=1, and N6=1

93) N1=1, N2=0, N3=0, N4=3, N5=2, and N6=1

94) N1=1, N2=0, N3=0, N4=3, N5=3, and N6=1

95) N1=1, N2=0, N3=0, N4=1, N5=1, and N6=1

96) N1=2, N2=0, N3=0, N4=1, N5=0, and N6=0

97) N1=2, N2=0, N3=1, N4=1, N5=0, and N6=0

98) N1=2, N2=0, N3=0, N4=2, N5=0, and N6=0

99) N1=2, N2=0, N3=0, N4=3, N5=0, and N6=0

100) N1=2, N2=0, N3=0, N4=1, N5=1, and N6=0

101) N1=2, N2=0, N3=0, N4=1, N5=2, and N6=0

102) N1=2, N2=0, N3=0, N4=1, N5=3, and N6=0

103) N1=2, N2=0, N3=0, N4=1, N5=0, and N6=1

104) N1=2, N2=0, N3=1, N4=2, N5=0, and N6=0

105) N1=2, N2=0, N3=1, N4=3, N5=0, and N6=0

106) N1=2, N2=0, N3=1, N4=1, N5=1, and N6=0

107) N1=2, N2=0, N3=1, N4=1, N5=2, and N6=0

108) N1=2, N2=0, N3=1, N4=1, N5=3, and N6=0

109) N1=2, N2=0, N3=1, N4=1, N5=0, and N6=1

110) N1=2, N2=0, N3=1, N4=2, N5=1, and N6=0

111) N1=2, N2=0, N3=1, N4=2, N5=1, and N6=1

112) N1=2, N2=0, N3=1, N4=2, N5=2, and N6=0

113) N1=2, N2=0, N3=1, N4=2, N5=2, and N6=1

114) N1=2, N2=0, N3=1, N4=2, N5=3, and N6=0

115) N1=2, N2=0, N3=1, N4=2, N5=3, and N6=1

116) N1=2, N2=0, N3=1, N4=2, N5=0, and N6=1

117) N1=2, N2=0, N3=1, N4=3, N5=1, and N6=0

118) N1=2, N2=0, N3=1, N4=3, N5=1, and N6=1

119) N1=2, N2=0, N3=1, N4=3, N5=2, and N6=0

120) N1=2, N2=0, N3=1, N4=3, N5=2, and N6=1

121) N1=2, N2=0, N3=1, N4=3, N5=3, and N6=0

122) N1=2, N2=0, N3=1, N4=3, N5=3, and N6=1

123) N1=2, N2=0, N3=1, N4=3, N5=0, and N6=1

124) N1=2, N2=0, N3=1, N4=1, N5=1, and N6=1

125) N1=2, N2=0, N3=1, N4=1, N5=2, and N6=1

126) N1=2, N2=0, N3=1, N4=1, N5=3, and N6=1

127) N1=2, N2=1, N3=0, N4=1, N5=0, and N6=0

128) N1=2, N2=1, N3=0, N4=2, N5=0, and N6=0

129) N1=2, N2=1, N3=0, N4=3, N5=0, and N6=0

130) N1=2, N2=1, N3=0, N4=1, N5=1, and N6=0

131) N1=2, N2=1, N3=0, N4=1, N5=2, and N6=0

132) N1=2, N2=1, N3=0, N4=1, N5=3, and N6=0

133) N1=2, N2=1, N3=0, N4=1, N5=0, and N6=1

134) N1=2, N2=1, N3=0, N4=2, N5=1, and N6=0

135) N1=2, N2=1, N3=0, N4=2, N5=1, and N6=1

136) N1=2, N2=1, N3=0, N4=2, N5=2, and N6=0

137) N1=2, N2=1, N3=0, N4=2, N5=2, and N6=1

138) N1=2, N2=1, N3=0, N4=2, N5=3, and N6=0

139) N1=2, N2=1, N3=0, N4=2, N5=3, and N6=1

140) N1=2, N2=1, N3=0, N4=2, N5=0, and N6=1

141) N1=2, N2=1, N3=0, N4=3, N5=1, and N6=0

142) N1=2, N2=1, N3=0, N4=3, N5=1, and N6=1

143) N1=2, N2=1, N3=0, N4=3, N5=2, and N6=0

144) N1=2, N2=1, N3=0, N4=3, N5=2, and N6=1

145) N1=2, N2=1, N3=0, N4=3, N5=3, and N6=0

146) N1=2, N2=1, N3=0, N4=3, N5=3, and N6=1

147) N1=2, N2=1, N3=0, N4=3, N5=0, and N6=1

148) N1=2, N2=1, N3=0, N4=1, N5=1, and N6=1

149) N1=2, N2=1, N3=0, N4=1, N5=2, and N6=1

150) N1=2, N2=1, N3=0, N4=1, N5=3, and N6=1

151) N1=2, N2=1, N3=1, N4=1, N5=0, and N6=0

152) N1=2, N2=1, N3=1, N4=2, N5=0, and N6=0

153) N1=2, N2=1, N3=1, N4=3, N5=0, and N6=0

154) N1=2, N2=1, N3=1, N4=1, N5=1, and N6=0

155) N1=2, N2=1, N3=1, N4=1, N5=2, and N6=0

156) N1=2, N2=1, N3=1, N4=1, N5=3, and N6=0

157) N1=2, N2=1, N3=1, N4=1, N5=0, and N6=1

158) N1=2, N2=1, N3=1, N4=2, N5=1, and N6=0

159) N1=2, N2=1, N3=1, N4=2, N5=1, and N6=1

160) N1=2, N2=1, N3=1, N4=2, N5=2, and N6=0

161) N1=2, N2=1, N3=1, N4=2, N5=2, and N6=1

162) N1=2, N2=1, N3=1, N4=2, N5=3, and N6=0

163) N1=2, N2=1, N3=1, N4=2, N5=3, and N6=1

164) N1=2, N2=1, N3=1, N4=2, N5=0, and N6=1

165) N1=2, N2=1, N3=1, N4=3, N5=1, and N6=0

166) N1=2, N2=1, N3=1, N4=3, N5=1, and N6=1

167) N1=2, N2=1, N3=1, N4=3, N5=2, and N6=0

168) N1=2, N2=1, N3=1, N4=3, N5=2, and N6=1

169) N1=2, N2=1, N3=1, N4=3, N5=3, and N6=0

170) N1=2, N2=1, N3=1, N4=3, N5=3, and N6=1

171) N1=2, N2=1, N3=1, N4=3, N5=0, and N6=1

172) N1=2, N2=1, N3=1, N4=1, N5=1, and N6=1

173) N1=2, N2=1, N3=1, N4=1, N5=2, and N6=1

174) N1=2, N2=1, N3=1, N4=1, N5=3, and N6=1

175) N1=2, N2=0, N3=0, N4=2, N5=1, and N6=0

176) N1=2, N2=0, N3=0, N4=2, N5=2, and N6=0

177) N1=2, N2=0, N3=0, N4=2, N5=3, and N6=0

178) N1=2, N2=0, N3=0, N4=2, N5=0, and N6=1

179) N1=2, N2=0, N3=0, N4=3, N5=1, and N6=0

180) N1=2, N2=0, N3=0, N4=3, N5=2, and N6=0

181) N1=2, N2=0, N3=0, N4=3, N5=3, and N6=0

182) N1=2, N2=0, N3=0, N4=3, N5=0, and N6=1

183) N1=2, N2=0, N3=0, N4=1, N5=2, and N6=1

184) N1=2, N2=0, N3=0, N4=1, N5=3, and N6=1

185) N1=2, N2=0, N3=0, N4=2, N5=1, and N6=1

186) N1=2, N2=0, N3=0, N4=2, N5=2, and N6=1

187) N1=2, N2=0, N3=0, N4=2, N5=3, and N6=1

188) N1=2, N2=0, N3=0, N4=3, N5=1, and N6=1

189) N1=2, N2=0, N3=0, N4=3, N5=2, and N6=1

190) N1=2, N2=0, N3=0, N4=3, N5=3, and N6=1

191) N1=2, N2=0, N3=0, N4=1, N5=1, and N6=1

192) N1=3, N2=0, N3=0, N4=1, N5=0, and N6=0

193) N1=3, N2=0, N3=1, N4=1, N5=0, and N6=0

194) N1=3, N2=0, N3=0, N4=2, N5=0, and N6=0

195) N1=3, N2=0, N3=0, N4=3, N5=0, and N6=0

196) N1=3, N2=0, N3=0, N4=1, N5=1, and N6=0

197) N1=3, N2=0, N3=0, N4=1, N5=2, and N6=0

198) N1=3, N2=0, N3=0, N4=1, N5=3, and N6=0

199) N1=3, N2=0, N3=0, N4=1, N5=0, and N6=1

200) N1=3, N2=0, N3=1, N4=2, N5=0, and N6=0

201) N1=3, N2=0, N3=1, N4=3, N5=0, and N6=0

202) N1=3, N2=0, N3=1, N4=1, N5=1, and N6=0

203) N1=3, N2=0, N3=1, N4=1, N5=2, and N6=0

204) N1=3, N2=0, N3=1, N4=1, N5=3, and N6=0

205) N1=3, N2=0, N3=1, N4=1, N5=0, and N6=1

206) N1=3, N2=0, N3=1, N4=2, N5=1, and N6=0

207) N1=3, N2=0, N3=1, N4=2, N5=1, and N6=1

208) N1=3, N2=0, N3=1, N4=2, N5=2, and N6=0

209) N1=3, N2=0, N3=1, N4=2, N5=2, and N6=1

210) N1=3, N2=0, N3=1, N4=2, N5=3, and N6=0

211) N1=3, N2=0, N3=1, N4=2, N5=3, and N6=1

212) N1=3, N2=0, N3=1, N4=2, N5=0, and N6=1

213) N1=3, N2=0, N3=1, N4=3, N5=1, and N6=0

214) N1=3, N2=0, N3=1, N4=3, N5=1, and N6=1

215) N1=3, N2=0, N3=1, N4=3, N5=2, and N6=0

216) N1=3, N2=0, N3=1, N4=3, N5=2, and N6=1

217) N1=3, N2=0, N3=1, N4=3, N5=3, and N6=0

218) N1=3, N2=0, N3=1, N4=3, N5=3, and N6=1

219) N1=3, N2=0, N3=1, N4=3, N5=0, and N6=1

220) N1=3, N2=0, N3=1, N4=1, N5=1, and N6=1

221) N1=3, N2=0, N3=1, N4=1, N5=2, and N6=1

222) N1=3, N2=0, N3=1, N4=1, N5=3, and N6=1

223) N1=3, N2=1, N3=0, N4=1, N5=0, and N6=0

224) N1=3, N2=1, N3=0, N4=2, N5=0, and N6=0

225) N1=3, N2=1, N3=0, N4=3, N5=0, and N6=0

226) N1=3, N2=1, N3=0, N4=1, N5=1, and N6=0

227) N1=3, N2=1, N3=0, N4=1, N5=2, and N6=0

228) N1=3, N2=1, N3=0, N4=1, N5=3, and N6=0

229) N1=3, N2=1, N3=0, N4=1, N5=0, and N6=1

230) N1=3, N2=1, N3=0, N4=2, N5=1, and N6=0

231) N1=3, N2=1, N3=0, N4=2, N5=1, and N6=1

232) N1=3, N2=1, N3=0, N4=2, N5=2, and N6=0

233) N1=3, N2=1, N3=0, N4=2, N5=2, and N6=1

234) N1=3, N2=1, N3=0, N4=2, N5=3, and N6=0

235) N1=3, N2=1, N3=0, N4=2, N5=3, and N6=1

236) N1=3, N2=1, N3=0, N4=2, N5=0, and N6=1

237) N1=3, N2=1, N3=0, N4=3, N5=1, and N6=0

238) N1=3, N2=1, N3=0, N4=3, N5=1, and N6=1

239) N1=3, N2=1, N3=0, N4=3, N5=2, and N6=0

240) N1=3, N2=1, N3=0, N4=3, N5=2, and N6=1

241) N1=3, N2=1, N3=0, N4=3, N5=3, and N6=0

242) N1=3, N2=1, N3=0, N4=3, N5=3, and N6=1

243) N1=3, N2=1, N3=0, N4=3, N5=0, and N6=1

244) N1=3, N2=1, N3=0, N4=1, N5=1, and N6=1

245) N1=3, N2=1, N3=0, N4=1, N5=2, and N6=1

246) N1=3, N2=1, N3=0, N4=1, N5=3, and N6=1

247) N1=3, N2=1, N3=1, N4=1, N5=0, and N6=0

248) N1=3, N2=1, N3=1, N4=2, N5=0, and N6=0

249) N1=3, N2=1, N3=1, N4=3, N5=0, and N6=0

250) N1=3, N2=1, N3=1, N4=1, N5=1, and N6=0

251) N1=3, N2=1, N3=1, N4=1, N5=2, and N6=0

252) N1=3, N2=1, N3=1, N4=1, N5=3, and N6=0

253) Ni=3, N2=1, N3=1, N4=1, N5=0, and N6=1

254) N1=3, N2=1, N3=1, N4=2, N5=1, and N6=0

255) N1=3, N2=1, N3=1, N4=2, N5=1, and N6=1

256) N1=3, N2=1, N3=1, N4=2, N5=2, and N6=0

257) N1=3, N2=1, N3=1, N4=2, N5=2, and N6=1

258) N1=3, N2=1, N3=1, N4=2, N5=3, and N6=0

259) N1=3, N2=1, N3=1, N4=2, N5=3, and N6=1

260) N1=3, N2=1, N3=1, N4=2, N5=0, and N6=1

261) N1=3, N2=1, N3=1, N4=3, N5=1, and N6=0

262) N1=3, N2=1, N3=1, N4=3, N5=1, and N6=1

263) Nl=3, N2=1, N3=1, N4=3, N5=2, and N6=0

264) N1=3, N2=1, N3=1, N4=3, N5=2, and N6=1

265) N1=3, N2=1, N3=1, N4=3, N5=3, and N6=0

266) N1=3, N2=1, N3=1, N4=3, N5=3, and N6=1

267) N1=3, N2=1, N3=1, N4=3, N5=0, and N6=1

268) N1=3, N2=1, N3=1, N4=1, N5=1, and N6=1

269) N1=3, N2=1, N3=1, N4=1, N5=2, and N6=1

270) N1=3, N2=1, N3=1, N4=1, N5=3, and N6=1

271) N1=3, N2=0, N3=0, N4=2, N5=1, and N6=0

272) N1=3, N2=0, N3=0, N4=2, N5=2, and N6=0

273) N1=3, N2=0, N3=0, N4=2, N5=3, and N6=0

274) N1=3, N2=0, N3=0, N4=2, N5=0, and N6=1

275) N1=3, N2=0, N3=0, N4=3, N5=1, and N6=0

276) N1=3, N2=0, N3=0, N4=3, N5=2, and N6=0

277) N1=3, N2=0, N3=0, N4=3, N5=3, and N6=0

278) N1=3, N2=0, N3=0, N4=3, N5=0, and N6=1

279) N1=3, N2=0, N3=0, N4=1, N5=2, and N6=1

280) N1=3, N2=0, N3=0, N4=1, N5=3, and N6=1

281) Nl=3, N2=0, N3=0, N4=2, N5=1, and N6=1

282) Nl=3, N2=0, N3=0, N4=2, N5=2, and N6=1

283) Nl=3, N2=0, N3=0, N4=2, N5=3, and N6=1

284) N1=3, N2=0, N3=0, N4=3, N5=1, and N6=1

285) N1=3, N2=0, N3=0, N4=3, N5=2, and N6=1

286) N1=3, N2=0, N3=0, N4=3, N5=3, and N6=1

287) N1=3, N2=0, N3=0, N4=1, N5=1, and N6=1

288) N1=4, N2=0, N3=0, N4=1, N5=0, and N6=0

289) N1=4, N2=0, N3=1, N4=1, N5=0, and N6=0

290) N1=4, N2=0, N3=0, N4=2, N5=0, and N6=0

291) N1=4, N2=0, N3=0, N4=3, N5=0, and N6=0

292) N1=4, N2=0, N3=0, N4=1, N5=1, and N6=0

293) N1=4, N2=0, N3=0, N4=1, N5=2, and N6=0

294) N1=4, N2=0, N3=0, N4=1, N5=3, and N6=0

295) N1=4, N2=0, N3=0, N4=1, N5=0, and N6=1

296) N1=4, N2=0, N3=1, N4=2, N5=0, and N6=0

297) N1=4, N2=0, N3=1, N4=3, N5=0, and N6=0

298) N1=4, N2=0, N3=1, N4=1, N5=1, and N6=0

299) N1=4, N2=0, N3=1, N4=1, N5=2, and N6=0

300) N1=4, N2=0, N3=1, N4=1, N5=3, and N6=0

301) N1=4, N2=0, N3=1, N4=1, N5=0, and N6=1

302) N1=4, N2=0, N3=1, N4=2, N5=1, and N6=0

303) N1=4, N2=0, N3=1, N4=2, N5=1, and N6=1

304) N1=4, N2=0, N3=1, N4=2, N5=2, and N6=0

305) N1=4, N2=0, N3=1, N4=2, N5=2, and N6=1

306) N1=4, N2=0, N3=1, N4=2, N5=3, and N6=0

307) N1=4, N2=0, N3=1, N4=2, N5=3, and N6=1

308) N1=4, N2=0, N3=1, N4=2, N5=0, and N6=1

309) N1=4, N2=0, N3=1, N4=3, N5=1, and N6=0

310) N1=4, N2=0, N3=1, N4=3, N5=1, and N6=1

311) N1=4, N2=0, N3=1, N4=3, N5=2, and N6=0

312) N1=4, N2=0, N3=1, N4=3, N5=2, and N6=1

313) N1=4, N2=0, N3=1, N4=3, N5=3, and N6=0

314) N1=4, N2=0, N3=1, N4=3, N5=3, and N6=1

315) N1=4, N2=0, N3=1, N4=3, N5=0, and N6=1

316) N1=4, N2=0, N3=1, N4=1, N5=1, and N6=1

317) N1=4, N2=0, N3=1, N4=1, N5=2, and N6=1

318) N1=4, N2=0, N3=1, N4=1, N5=3, and N6=1

319) N1=4, N2=1, N3=0, N4=1, N5=0, and N6=0

320) N1=4, N2=1, N3=0, N4=2, N5=0, and N6=0

321) N1=4, N2=1, N3=0, N4=3, N5=0, and N6=0

322) N1=4, N2=1, N3=0, N4=1, N5=1, and N6=0

323) N1=4, N2=1, N3=0, N4=1, N5=2, and N6=0

324) N1=4, N2=1, N3=0, N4=1, N5=3, and N6=0

325) N1=4, N2=1, N3=0, N4=1, N5=0, and N6=1

326) N1=4, N2=1, N3=0, N4=2, N5=1, and N6=0

327) N1=4, N2=1, N3=0, N4=2, N5=1, and N6=1

328) N1=4, N2=1, N3=0, N4=2, N5=2, and N6=0

329) N1=4, N2=1, N3=0, N4=2, N5=2, and N6=1

330) N1=4, N2=1, N3=0, N4=2, N5=3, and N6=0

331) N1=4, N2=1, N3=0, N4=2, N5=3, and N6=1

332) N1=4, N2=1, N3=0, N4=2, N5=0, and N6=1

333) N1=4, N2=1, N3=0, N4=3, N5=1, and N6=0

334) N1=4, N2=1, N3=0, N4=3, N5=1, and N6=1

335) N1=4, N2=1, N3=0, N4=3, N5=2, and N6=0

336) N1=4, N2=1, N3=0, N4=3, N5=2, and N6=1

337) N1=4, N2=1, N3=0, N4=3, N5=3, and N6=0

338) N1=4, N2=1, N3=0, N4=3, N5=3, and N6=1

339) N1=4, N2=1, N3=0, N4=3, N5=0, and N6=1

340) N1=4, N2=1, N3=0, N4=1, N5=1, and N6=1

341) N1=4, N2=1, N3=0, N4=1, N5=2, and N6=1

342) N1=4, N2=1, N3=0, N4=1, N5=3, and N6=1

343) N1=4, N2=1, N3=1, N4=1, N5=0, and N6=0

344) N1=4, N2=1, N3=1, N4=2, N5=0, and N6=0

345) N1=4, N2=1, N3=1, N4=3, N5=0, and N6=0

346) N1=4, N2=1, N3=1, N4=1, N5=1, and N6=0

347) N1=4, N2=1, N3=1, N4=1, N5=2, and N6=0

348) N1=4, N2=1, N3=1, N4=1, N5=3, and N6=0

349) N1=4, N2=1, N3=1, N4=1, N5=0, and N6=1

350) N1=4, N2=1, N3=1, N4=2, N5=1, and N6=0

351) N1=4, N2=1, N3=1, N4=2, N5=1, and N6=1

352) N1=4, N2=1, N3=1, N4=2, N5=2, and N6=0

353) N1=4, N2=1, N3=1, N4=2, N5=2, and N6=1

354) N1=4, N2=1, N3=1, N4=2, N5=3, and N6=0

355) N1=4, N2=1, N3=1, N4=2, N5=3, and N6=1

356) N1=4, N2=1, N3=1, N4=2, N5=0, and N6=1

357) N1=4, N2=1, N3=1, N4=3, N5=1, and N6=0

358) N1=4, N2=1, N3=1, N4=3, N5=1, and N6=1

359) N1=4, N2=1, N3=1, N4=3, N5=2, and N6=0

360) N1=4, N2=1, N3=1, N4=3, N5=2, and N6=1

361) N1=4, N2=1, N3=1, N4=3, N5=3, and N6=0

362) N1=4, N2=1, N3=1, N4=3, N5=3, and N6=1

363) N1=4, N2=1, N3=1, N4=3, N5=0, and N6=1

364) N1=4, N2=1, N3=1, N4=1, N5=1, and N6=1

365) N1=4, N2=1, N3=1, N4=1, N5=2, and N6=1

366) N1=4, N2=1, N3=1, N4=1, N5=3, and N6=1

367) N1=4, N2=0, N3=0, N4=2, N5=1, and N6=0

368) N1=4, N2=0, N3=0, N4=2, N5=2, and N6=0

369) N1=4, N2=0, N3=0, N4=2, N5=3, and N6=0

370) N1=4, N2=0, N3=0, N4=2, N5=0, and N6=1

371) N1=4, N2=0, N3=0, N4=3, N5=1, and N6=0

372) N1=4, N2=0, N3=0, N4=3, N5=2, and N6=0

373) N1=4, N2=0, N3=0, N4=3, N5=3, and N6=0

374) N1=4, N2=0, N3=0, N4=3, N5=0, and N6=1

375) N1=4, N2=0, N3=0, N4=1, N5=2, and N6=1

376) N1=4, N2=0, N3=0, N4=1, N5=3, and N6=1

377) N1=4, N2=0, N3=0, N4=2, N5=1, and N6=1

378) N1=4, N2=0, N3=0, N4=2, N5=2, and N6=1

379) N1=4, N2=0, N3=0, N4=2, N5=3, and N6=1

380) N1=4, N2=0, N3=0, N4=3, N5=1, and N6=1

381) N1=4, N2=0, N3=0, N4=3, N5=2, and N6=1

382) N1=4, N2=0, N3=0, N4=3, N5=3, and N6=1

383) N1=4, N2=0, N3=0, N4=1, N5=1, and N6=1

Some preferred embodiments of the present invention and of embodiments ET1, ET2, ET3, ET7 and ET8 are shown below and designated as embodiments “ETS 1.X” wherein X=1, 2, 3, 4, 5, 6 . . . 295 and is the number of the structure below:

wherein A1, A2, and A3 designate targeting ligands, which may be the same or different; and B, B1, and B2, designate triggers that increase the effector activity PA and may be the same or different, and C designates a masked intracellular transport ligand; and D designates an intracellular trapping ligand; or a masked intracellular trapping ligand; and E, E1, and E2 designate effector agents which may be the same or different, and F designates a trigger that when activated decreases the effector activity PA; and L designates a linker; which may be the same or different from other linkers; and the lines represent the connectivity of the above components:

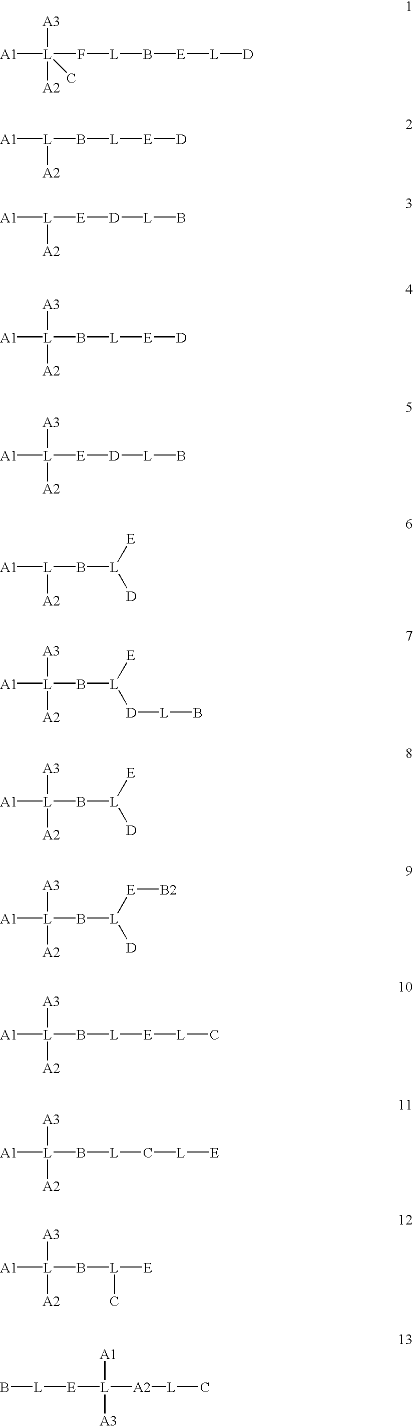

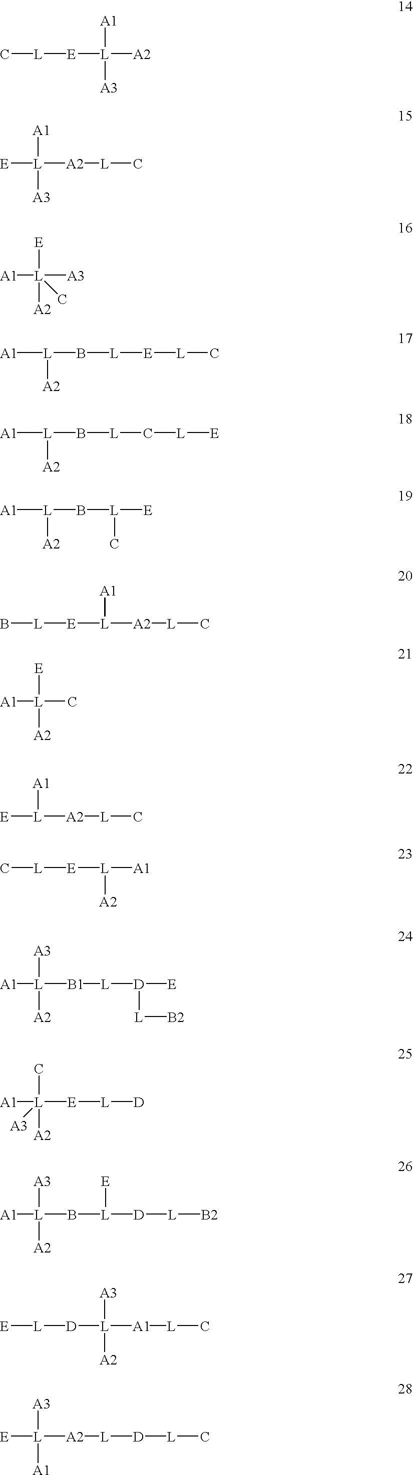

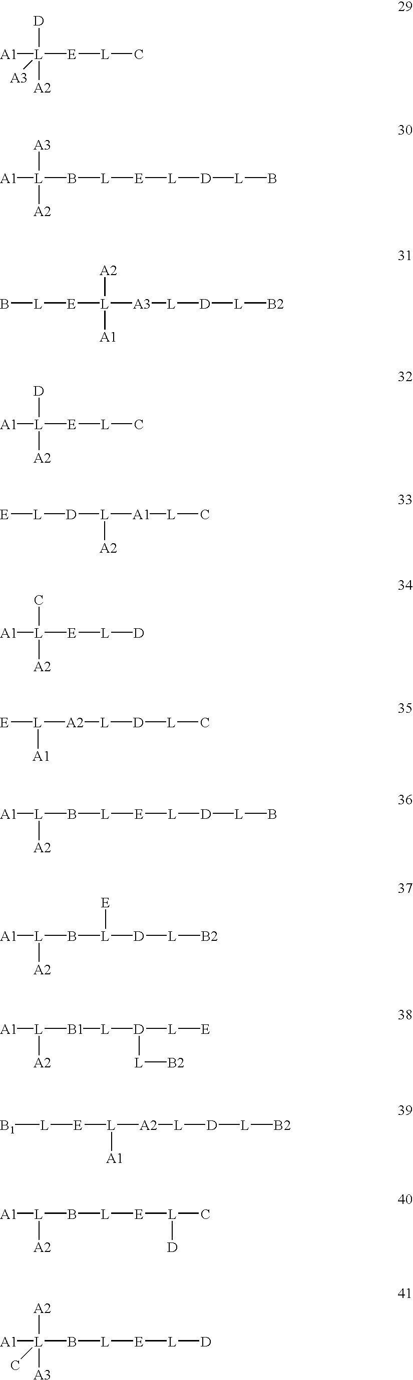

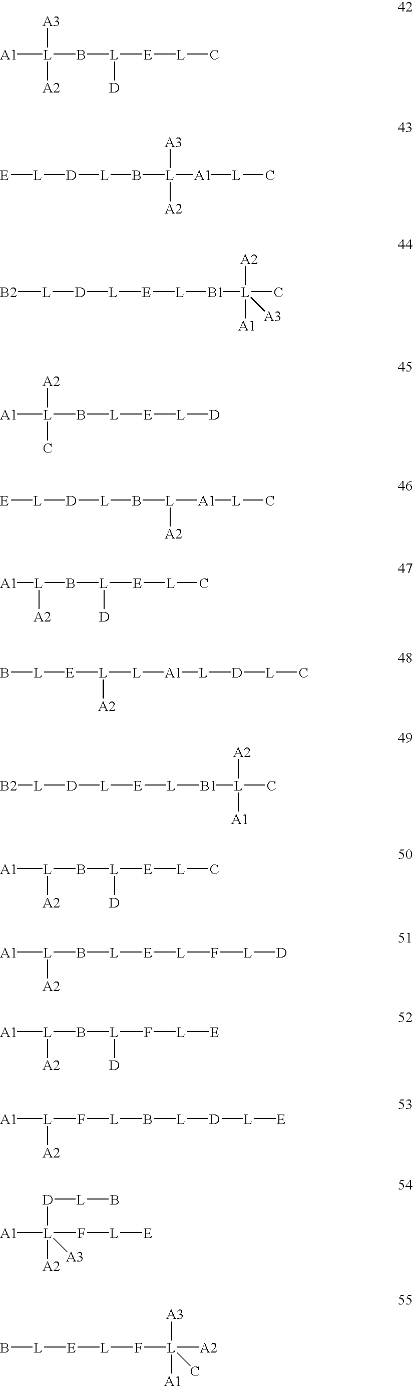

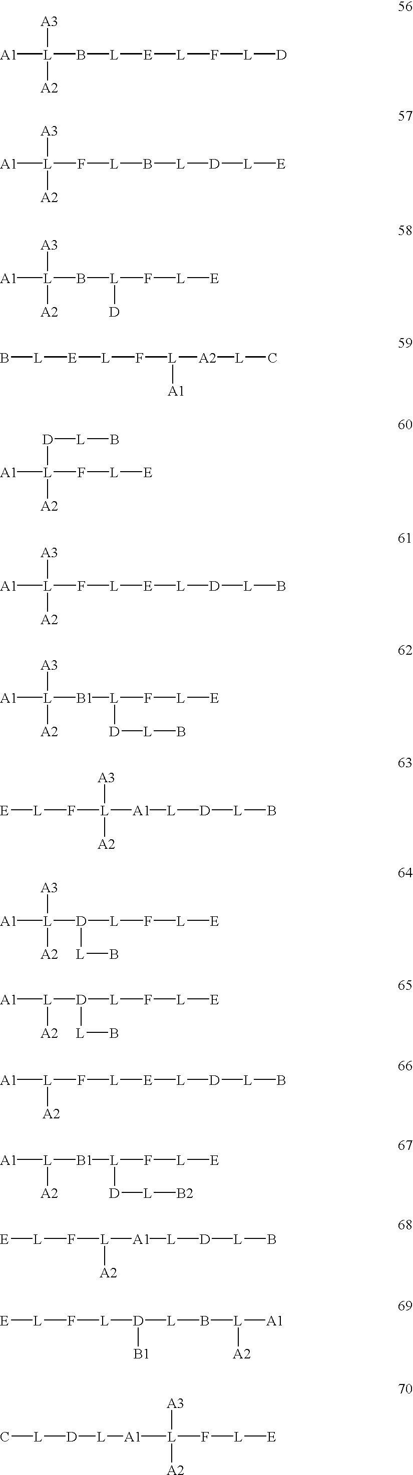

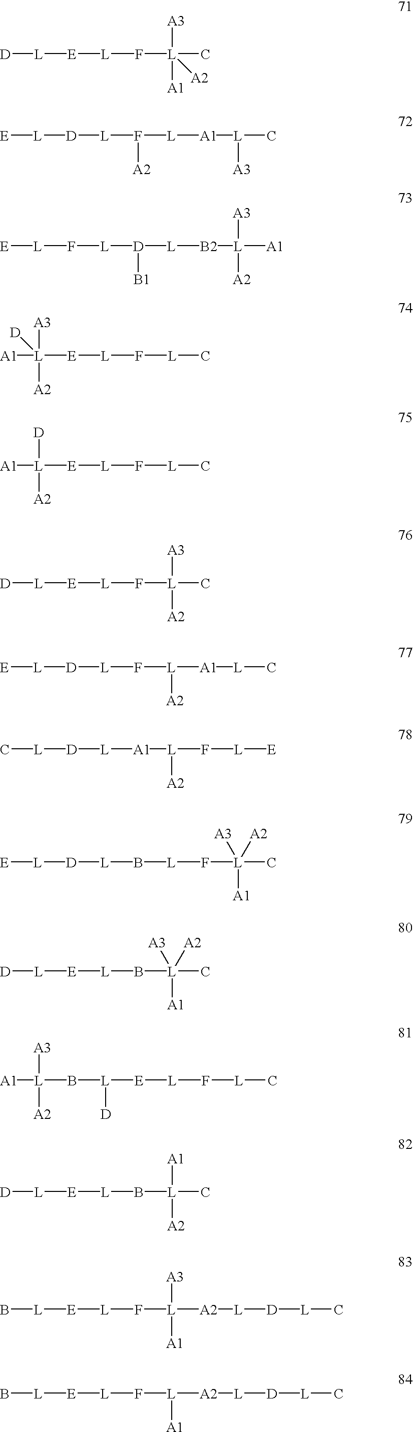

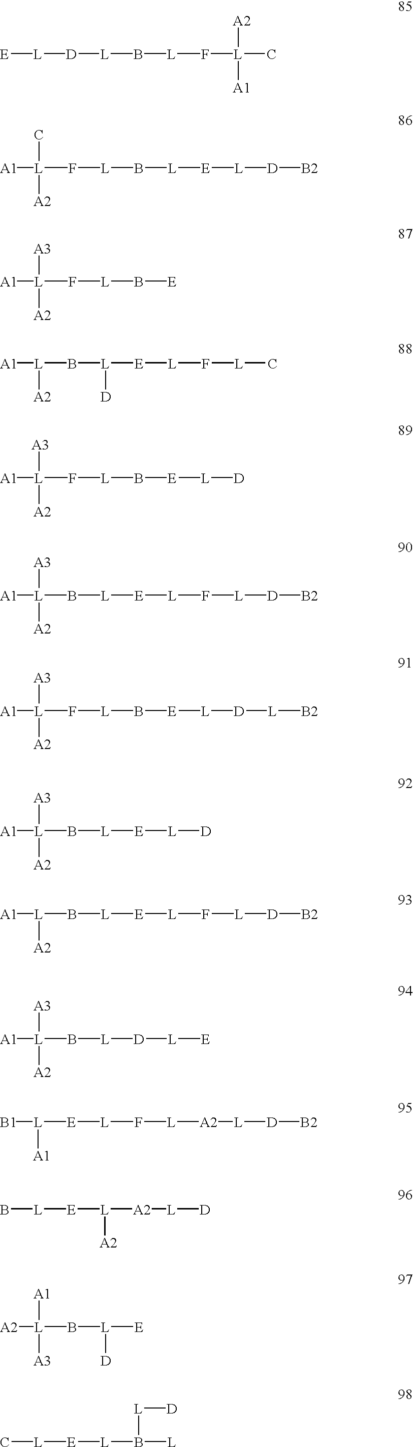

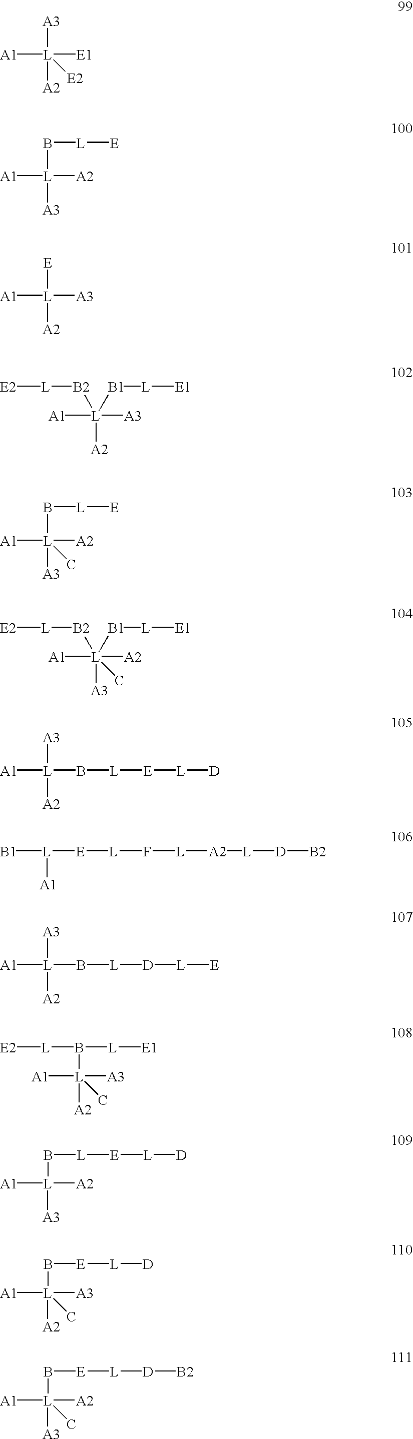

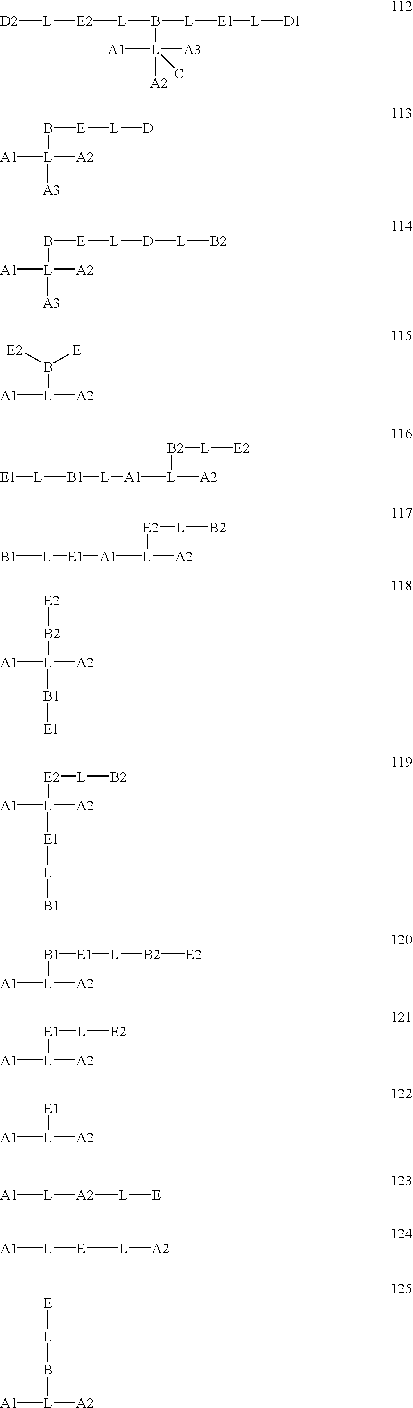

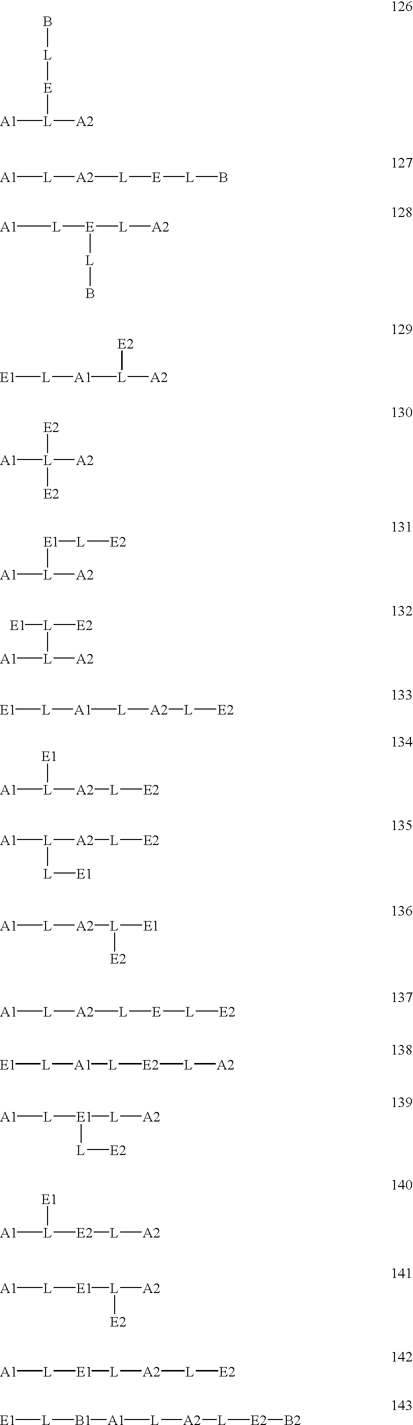

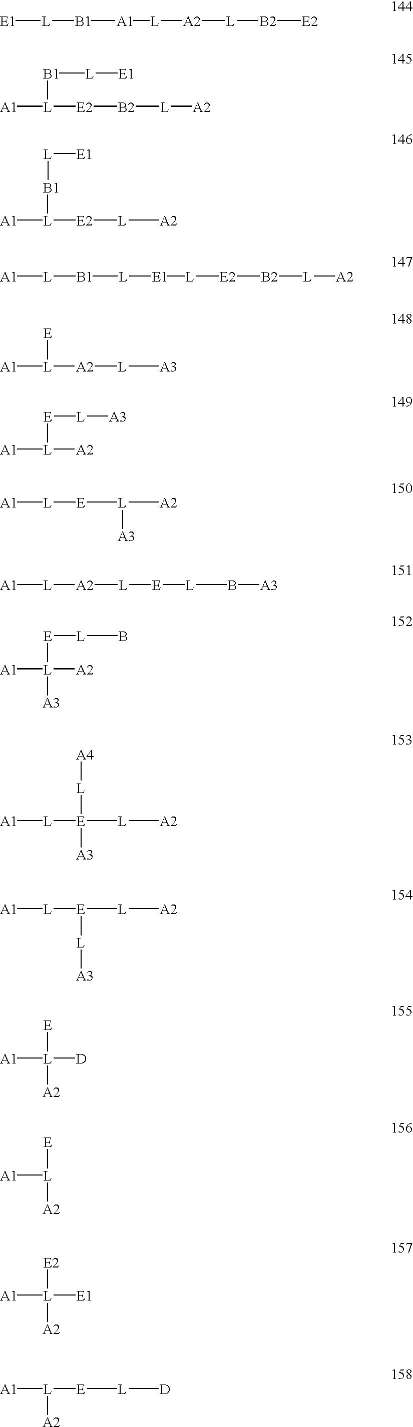

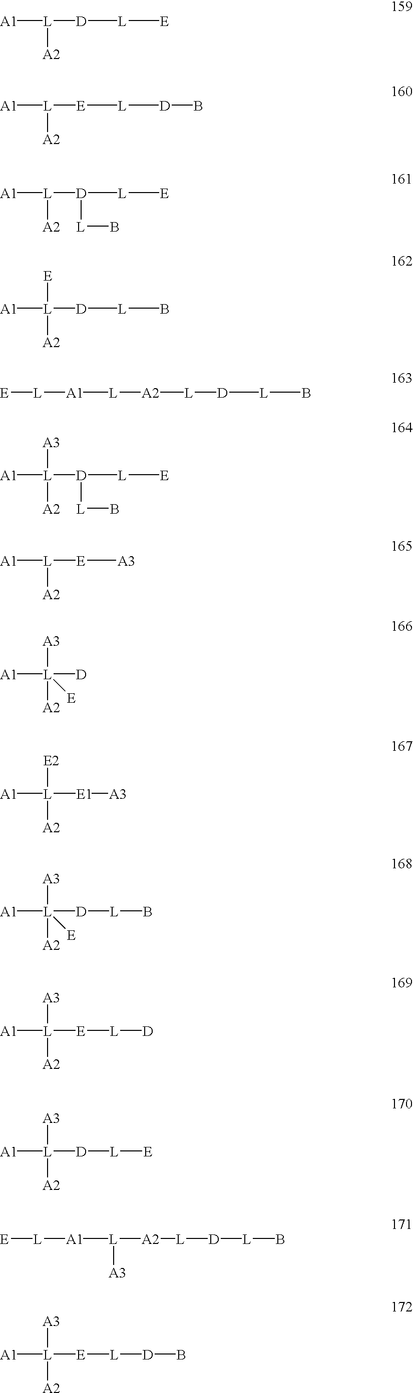

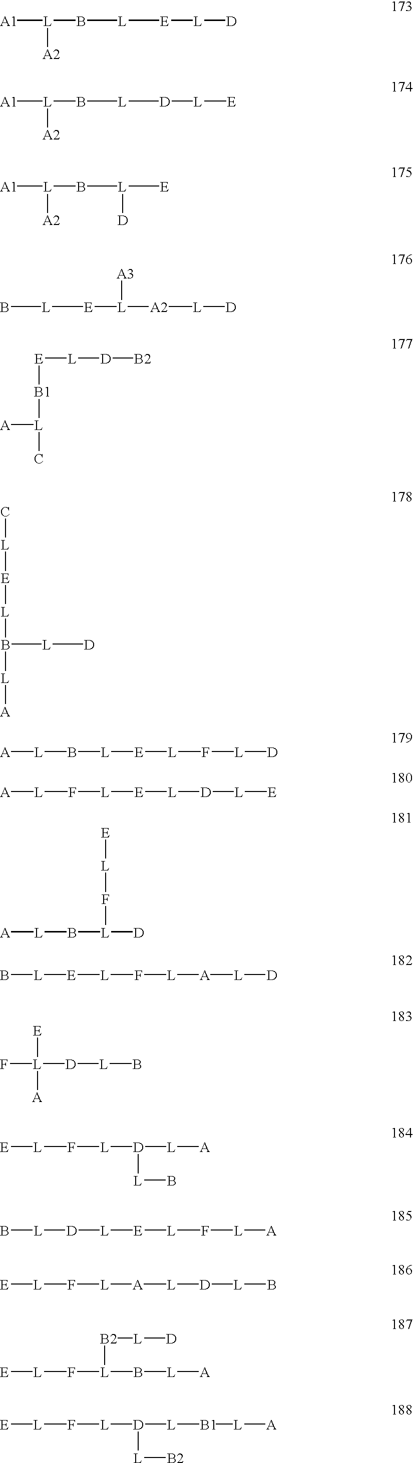

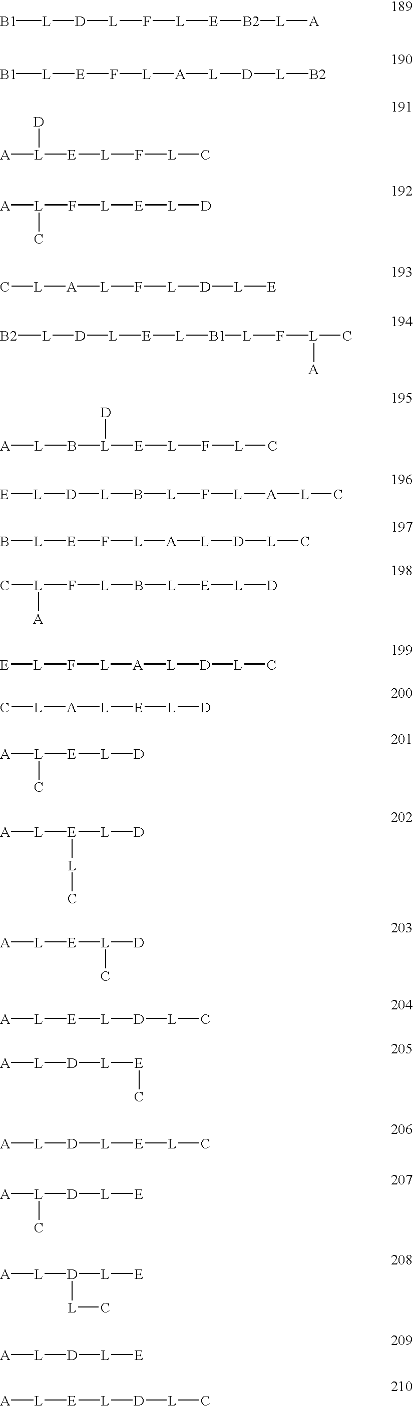

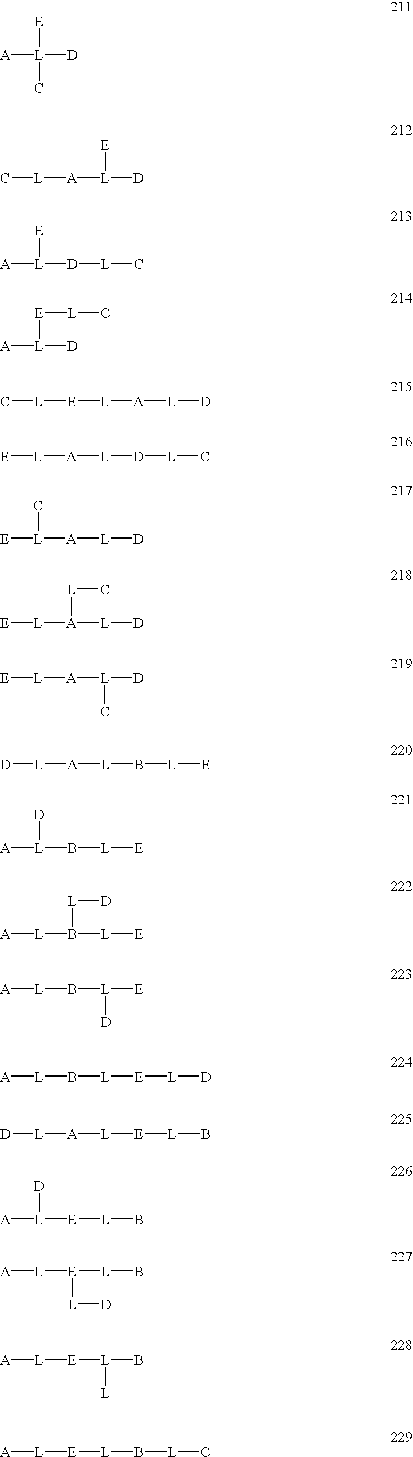

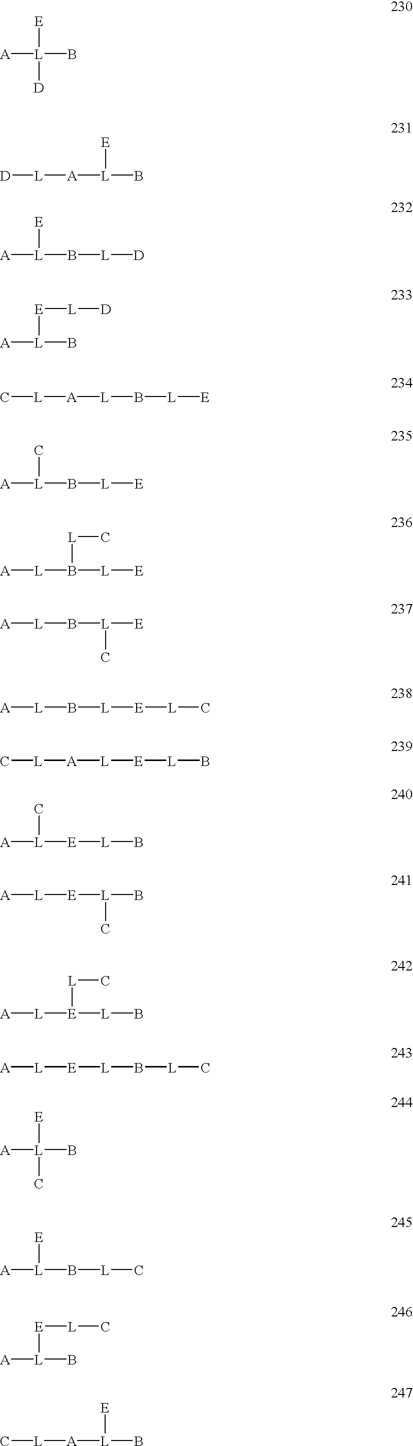

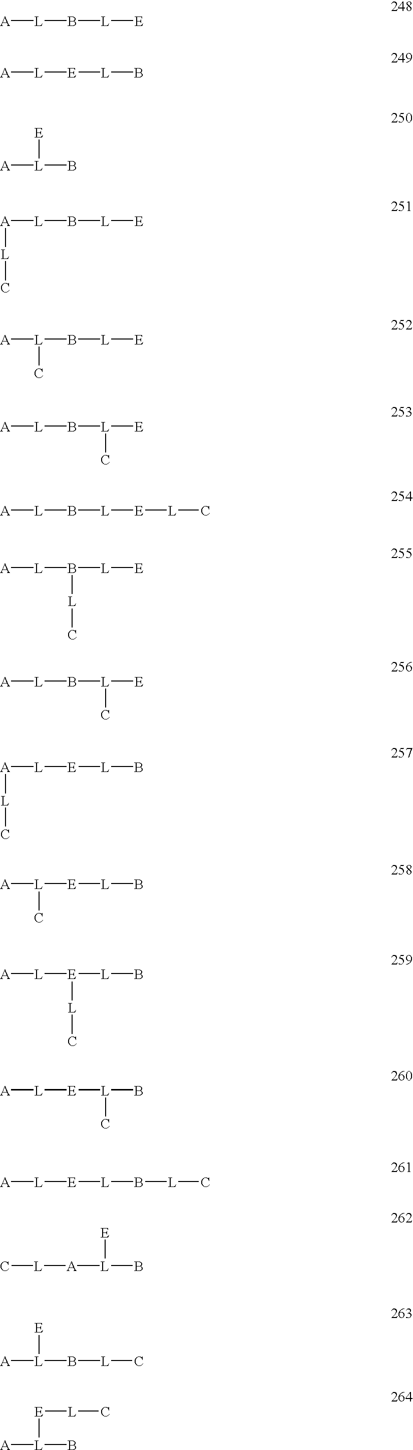

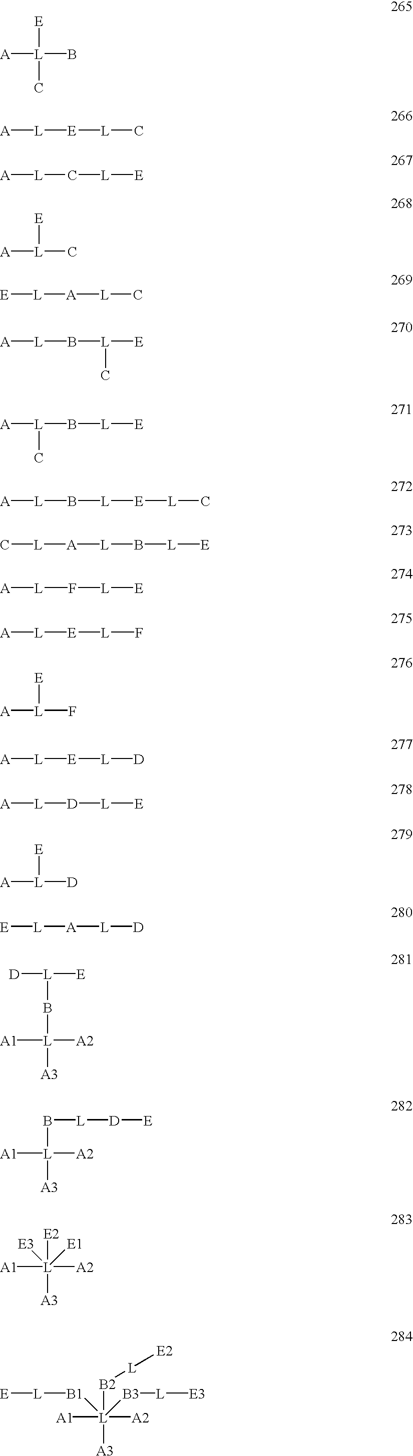

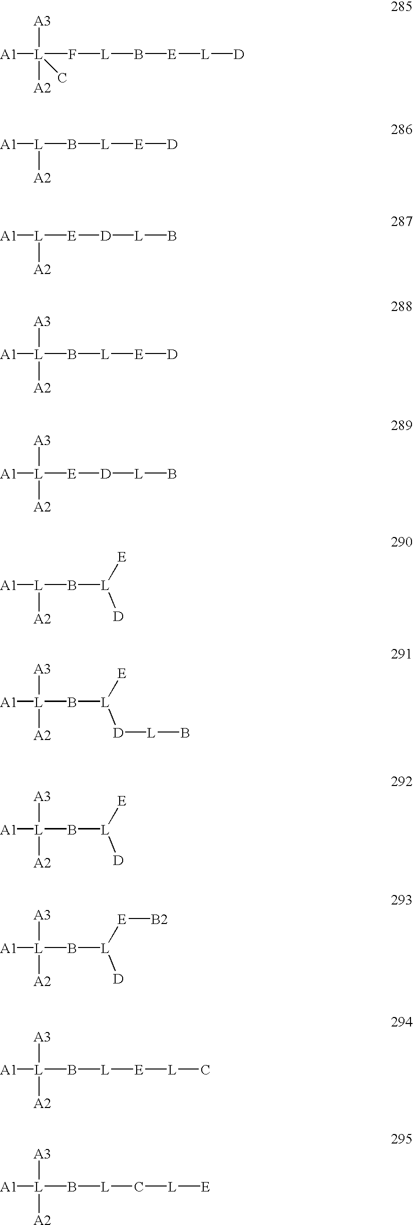

A preferred embodiment comprises ET with one selective targeting ligand at least one masked intracellular transport ligand or where (N1=1 and N2≠0).

A preferred embodiment comprises ET with one selective targeting ligand at least one detoxification trigger or where (N1=1 and N3≠0).

A preferred embodiment comprises ET with one selective targeting ligand at least one intracellular trapping ligand or masked intracellular trapping ligand or where (N1=1 and N6≠0).

A preferred embodiment comprises ET with one selective targeting ligand at least one trigger or where (N1=1 and N5≠0).

A preferred embodiment comprises ET with one selective targeting ligand at least one masked intracellular transport ligand or where (N1=2 and N2≠0).

A preferred embodiment comprises ET with one selective targeting ligand one non-selective targeting ligand.

A preferred embodiment comprises ET with one selective targeting ligand at least one detoxification trigger or where (N1=2 and N3≠0).

A preferred embodiment comprises ET with one selective targeting ligand one non-selective targeting ligand.

A preferred embodiment comprises ET with two targeting ligands and at least one intracellular trapping ligand or masked intracellular trapping ligand or where (N1=2 and N6≠0).

A preferred embodiment comprises ET with one selective targeting ligand one non-selective targeting ligand.

A preferred embodiment comprises ET with two targeting ligands and at least

one trigger or where (N1=2 and N5≠0).

A preferred embodiment comprises ET with one selective targeting ligand one non-selective targeting ligand.

A preferred embodiment comprises ET with three targeting ligands and at least one masked intracellular transport ligand or where (N1=3 and N2≠0).

A preferred embodiment comprises ET with three targeting ligands and at least one detoxification trigger or where (N1=3 and N3≠0).

A preferred embodiment comprises ET with three targeting ligands and at least one intracellular trapping ligand or masked intracellular trapping ligand or where (N1=3 and N6≠0).

A preferred embodiment comprises ET with three targeting ligands and at least one trigger or where (N1=3 and N5≠0).

A preferred embodiment comprises ET with 4 targeting ligands or where N1=4.

Another preferred embodiment of the present invention is comprised of at least one molecule ET that has been covalently linked to a second molecule that binds to a receptor present in increased amounts at a target cell compared to at a non-target cell; and wherein said second molecule is comprised of a monoclonal antibody, or targeting receptor binding fragment of a monoclonal antibody, or an analog or derivative thereof which bears amino acid sequence similarity to portions of a monoclonal antibody. Also the second molecule coupled to ET can be comprised of a natural protein, or a complex of natural proteins, or a protein, or a naturally occurring polymer that binds to the targeting receptor.

The present invention also comprises (embodiment ET9) a compound ET wherein E is comprised of one or more effector agents designated as E1 . . . En wherein n=1, 2, 3, 4, or 5 or about 5 and wherein these effector agents have pharmacological activity referred to as “PA”; and wherein T is a targeting agent which comprises targeting ligands or targeting ligands and triggers; and wherein T increases the pharmacological activity PA to a target cell compared to non-target cells;

and wherein a targeting ligand is a group that binds selectively to a structure associated with the target referred to as a “targeting receptor”;

and wherein a trigger is a group that upon in vivo modification by biomolecules referred to as “triggering agents” becomes activated and modulates the activity of ET;

wherein at the target cells there are present “m” different types of target molecules designated as (p1 . . . pm); at least one of which is present at increased amounts compared to at a non-target cell, and wherein the type of the targeting molecule which is increased on the target cells compared to a non-target cell can be different for a different non-target cell;

and wherein at non-target cells there can be present the same types of target molecules (p1 . . . pm);

and wherein ET is able to “interact with” each of the targeting molecules (p1 . . . pm); wherein the term “interact with” means to bind to a targeting receptor or to have a trigger modified by a triggering agent;

and wherein the number m is 2, 3, 4, 5, 6, 7, 8, 9, 10, 11, 12, 13, 14, 15, 16, 17,18, 19, or 20, or about 20.

In a preferred embodiment (embodiment ET10) the number m is about 2 to 5.

In a preferred embodiment (embodiment ET 11) of the present invention, the target is comprised of a tumor, or tumor cell, or components of a tumor, or biomolecules present in the microenvironment of the tumor, or stromal cells present in a tumor, and the effector agent or the pharmacalogical activity PA can evoke or can contribute to tumor cell killing and/or comprises a diagnostic agent.

In a preferred embodiment of the invention and of the embodiments ET1, and ET2, and ET3,and ET4, ET5,and ET6,and ET7,and ET8, and (ET8.X, wherein X=1, 2, 3 . . . 383), and ET9, and ET10, and (ETS1.X where X=1, 2, 3, 4, . . . 295); designated respectively as embodiments ET12.ET1, and ET12.ET2, and ET12.ET3, and ET12.ET3, and ET12.ET4, and ET12.ET5, and ET12.ET5 and ET12.ET6, and ET12.ET7, and ET12.ET8 and (ET12.ET8.X with X=1, 2, 3, 4 . . . 383) and ET12.ET9; and ET12.ET10, and ET12.ET8.X with X=1, 2, 3, 4, 5 . . . 295);

the target is comprised of a tumor, or tumor cell, or components of a tumor, or biomolecules present in the microenvironment of the tumor, or stromal cells present in a tumor, and the effector agent can evoke or can contribute to tumor cell killing and/or comprises a diagnostic agent.

Some preferred embodiments of the invention and of embodiments ET12.ET1, and ET12.ET2, and ET12.ET3, and ET12.ET3, and ET12.ET4, and ET12.ET5, and ET12.ET5 and ET12.ET6, and ET12.ET7, and ET12.ET8 and (ET12.ET8.X with X=1, 2, 3, 4 . . . 383) and ET12.ET9; and ET12.ET10, and ET12.ET8.X with X=1, 2, 3, 4, 5 . . . 295); follow:

A preferred embodiment is an anti-cancer drug ET comprised of effector agents that are cytotoxic drugs, and/or radionuclides, and/or immunostimulatory drugs. A preferred embodiment is an anti-cancer drug ET comprised of effector agents that are cytotoxic drugs. A preferred embodiment is an anti-cancer drug ET comprised of effector agents that are radionuclides. A preferred embodiment is an anti-cancer drug ET comprised of effector agents that are cytotoxic drugs that produce synergistic cytotoxicity. A preferred embodiment is an anti-cancer drug ET comprised of effector agents that stimulate the immune system. A preferred embodiment is an anti-cancer drug ET comprised of effector agents that stimulate the innate immune system. A preferred embodiment is an anti-cancer drug ET comprised of effector agents that irreversibly chemically modify one or more tumor components. A preferred embodiment is an anti-cancer drug ET comprised of effector agents that irreversibly chemically modify one or more tumor components that are present in increased amounts in tumor cells or in the microenvironment of tumors compared to vital normal cells. A preferred embodiment is an anti-cancer drug ET comprised of effector agents that potentiates the cytotoxic activity of a second effector agent. A preferred embodiment is an anti-cancer drug ET with an effector agent that comprises an inhibitor to multi-drug transporter proteins. A preferred embodiment is an anti-cancer drug ET with an effector agent that comprises an inhibitor to a membrane protein transporter that faciltates uptake of a nutrient or biomolecule into tumor cells. In a preferred embodiment ET is an anti-cancer drug with an effector agent that comprises an inhibitor to nucleoside transporter proteins.

In a preferred embodiment ET is an anti-cancer drug with targeting ligands that selectively bind to target receptors on the surface of the tumor cell or in the microenvironment of the tumor cell wherein the concentration of the target receptor is greater on the surface of the tumor cell or in the microenvironment of the tumor cell than on the surface or in the microenvironment of normal cells especially vital normal cells.

In a preferred embodiment ET is an anti-cancer drug with an intracellular trapping ligand that selectively binds to one or more intracellular receptors wherein the concentration of the intracellular receptors is greater in tumor cells then in vital normal cells.

In a preferred embodiment ET is an anti-cancer drug with a trigger that increases cytotoxicity of the drug upon in vivo modification and wherein the in vivo modification that activates the trigger is caused by an enzyme or enzymatic activity that is increased at tumor cells or decreased at vital normal cells.

In a preferred embodiment ET is an anti-cancer drug with a trigger that decreases the cytotoxicity of the drug upon in vivo modification and wherein the in vivo modification that activates the trigger is caused by an enzyme or enzymatic activity that is decreased at tumor cells or increased at vital normal cells.

In a preferred embodiment ET is an anti-cancer drug in which the intracellular transport ligand binds to a molecule referred to as a “transporter molecule” to form a complex and wherein this complex binds to a target cell receptor that actively transports bound ligands into the tumor cell. In a preferred embodiment ET is an anti-cancer drug for which the concentration of transporter molecules is increased at the surface of tumor cells compared to vital normal cells.

In a preferred embodiment of the present invention ET is comprised of an anti-cancer drug with two targeting ligands that selectively bind to target receptors on the surface of the tumor cell or in the microenvironment of the tumor cell wherein the concentration of the target receptors is greater on the surface of the tumor cell or in the microenvironment of the tumor cell than on the surface or in the microenvironment of vital normal cells or normal cells. In a preferred embodiment the targeting ligands are the same. In a preferred embodiment the targeting ligands are different and bind to different types of targeting receptors.

In a preferred embodiment ET is an anti-cancer drug with three targeting ligands that selectively bind to target receptors on the surface of the tumor cell or in the microenvironment of the tumor cell wherein the concentration of the target receptors is greater on the surface of the tumor cell or in the microenvironment of the tumor cell than on the surface or in the microenvironment of normal cells or vital normal cells. In a preferred embodiment the targeting ligands are the same. In a preferred embodiment the targeting ligands are different and bind to different types of targeting receptors.

In a preferred embodiment ET is an anti-cancer drug with four targeting ligands that selectively bind to target receptors on the surface of the tumor cell or in the microenvironment of the tumor cell wherein the concentration of the target receptors is greater on the surface of the tumor cell or in the microenvironment of the tumor cell than on the surface or in the microenvironment of normal cells or vital normal cells. In a preferred embodiment the targeting ligands are the same. In a preferred embodiment the targeting ligands are different and bind to different types of targeting receptors.

In another preferred embodiment ET is an anti-cancer drug comprised of a compound with two or more targeting ligands wherein at least one of the targeting ligands binds to a target receptor on the surface of the target cell or in the microenvironment of the target cell wherein the target has an increased amount of that target receptor compared to a non-target cell that binds to a second targeting ligand of the compound. A preferred embodiment of this embodiment comprises a compound with two different targeting ligands that bind to two different targeting receptors. Another preferred embodiment of this embodiment comprises a compound with three different targeting ligands that bind to three different targeting receptors. Another preferred embodiment of this embodiment comprises a compound with four different targeting ligands that bind to four different targeting receptors. In a preferred embodiment the drug binds to at most one type of receptor present on normal cells.

In another preferred embodiment the anti-cancer compound ET is comprised of one tumor-selective targeting ligand at least one masked intracellular transport ligand or where (N1=1 and N2≠0).

In another preferred embodiment the anti-cancer compound ET is comprised of one tumor-selective targeting ligand at least one detoxification trigger or where (N1=1 and N3≠0).

In another preferred embodiment the anti-cancer compound ET is comprised of one tumor-selective targeting ligand at least one intracellular trapping ligand or masked intracellular trapping ligand or where (N1=1 and N6≠0).

In another preferred embodiment the anti-cancer compound ET is comprised of one tumor-selective targeting ligand at least one trigger or where (N1=1 and N5≠0).

In another preferred embodiment the anti-cancer compound ET is comprised of two targeting ligands and at least one masked intracellular transport ligand or where (N1=2 and N2≠0). In a preferred embodiment of this the anti-cancer compound ET is comprised of one selective targeting ligand one non-selective targeting ligand. In another embodiment of this both targeting ligands are tumor-selective.

In another preferred embodiment the anti-cancer compound ET is comprised of two targeting ligands and at least one detoxification trigger or where (N 1=2 and N3≠0). In a preferred embodiment of this the anti-cancer compound ET is comprised of one selective targeting ligand one non-selective targeting ligand. In another embodiment of this, both targeting ligands are tumor-selective.

In another preferred embodiment the anti-cancer compound ET is comprised of two targeting ligands and at least one intracellular trapping ligand or masked intracellular trapping ligand or where (N1=2 and N6≠0). In a preferred embodiment of this the anti-cancer compound ET is comprised of one selective targeting ligand one non-selective targeting ligand. In another embodiment of this, both targeting ligands are tumor-selective.

In another preferred embodiment the anti-cancer compound ET is comprised of two targeting ligands and at least one trigger or where (N1=2 and N5≠0). In a preferred embodiment of this the anti-cancer compound ET is comprised of one selective targeting ligand one non-selective targeting ligand. In another embodiment of this, both targeting ligands are tumor-selective.

In another preferred embodiment the anti-cancer compound ET is comprised of three targeting ligands and at least one masked intracellular transport ligand or where (N1=3 and N2≠0).

In another preferred embodiment the anti-cancer compound ET is comprised of three targeting ligands and at least one detoxification trigger or where (N1=3 and N3≠0).

In another preferred embodiment the anti-cancer compound ET is comprised of three targeting ligands and at least one intracellular trapping ligand or masked intracellular trapping ligand or where (N1=3 and N6≠0). In another preferred embodiment the anti-cancer compound ET is comprised of three targeting ligands and at least one trigger or where (N1=3 and N5≠0). In another preferred embodiment the anti-cancer compound ET is comprised of 4 targeting ligands or where (N1=4).

In another preferred embodiment the anti-cancer compound ET is comprised of two identical tumor-selective targeting ligands and one effector agent or where N1=2, and N2=0, and N3=0, and N4=1, and N5=0, and N6=0. In a preferred embodiment of this, the effector agent is a cytotoxic drug. In another embodiment of this, the effector agent is comprised of a radionuclide.

In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of two different tumor-selective targeting ligands and one effector agent or where N1=2, and N2=0, and N3=0, and N4=1, and N5=0, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of two identical tumor-selective targeting ligands and one effector agent and one trigger that increases the toxicity of the effector agent or where N1=2, and N2=0, and N3=0, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of two different tumor-selective targeting ligands that bind to different tumor-selective receptors and one effector agent and one trigger that increases the toxicity of the effector agent or where N1=2, and N2=0, and N3=0, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of two identical tumor-selective targeting ligands and one effector agent and one trigger that increases the toxicity of the effector and one masked intracellular transporter ligand; or where N1=2, and N2=1, and N3=0, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of two different tumor-selective targeting ligands that bind to two different tumor-selective receptors and one effector agent and one trigger that increases the toxicity of the effector and one masked intracellular transporter ligand; or where N1=2, and N2=1, and N3=0, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of two different tumor-selective targeting ligands and one effector agent and one trigger that increases the toxicity of the effector agent and one masked intracellular transport ligand, and one intracellular trapping ligand or one masked intracellular trapping ligand or where N1=2, and N2=1, and N3=0, and N4=1, and N5=1, and N6=1. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of two different tumor-selective targeting ligands and one effector agent and one trigger that increases the toxicity of the effector agent, and one trigger that decrease the toxicity of the effector agent, and one masked intracellular transport ligand or where N1=2, and N2=1, and N3=1, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of two different tumor-selective targeting ligands and one effector agent and one trigger that increases the toxicity of the effector agent, and one trigger that decrease the toxicity of the effector agent, and one masked intracellular transport ligand one intracellular trapping ligand or masked intracellular trapping ligand. or where N1=2, and N2=1, and N3=1, and N4=1, and N5=1, and N6=1. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of three different tumor-selective targeting ligands to three different targeting receptors and one effector agent or where N1=3, and N2=0, and N3=0, and N4=1, and N5=0, and N6=0. In a preferred embodiment of this the effector agent is a cytotoxic drug In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of three different tumor-selective targeting ligands to three different targeting receptors and one effector agent and one trigger that increases the toxicity of the effector agent, or where N1=3, and N2=0, and N3=0, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of three different tumor-selective targeting ligands to three different targeting receptors and one effector agent and one trigger that increases the toxicity of the effector agent and one masked intracellular transporter ligand or where N1=3, and N2=1, and N3=0, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of three different tumor-selective targeting ligands to three different targeting receptors and one effector agent and one trigger that increases the toxicity of the effector agent and one masked intracellular transport ligand one intracellular trapping ligand or masked intracellular trapping ligand, or where N1=3, and N2=1, and N3=0, and N4=1, and N5=1, and N6=1. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of three different tumor-selective targeting ligands to three different targeting receptors and one effector agent and one trigger that increases the toxicity of the effector agent and one masked intracellular transport ligand one intracellular trapping ligand or masked intracellular trapping ligand, and one trigger that decreases the toxicity of the effector agent or where N1=3, and N2=1, and N3=1, and N4=1, and N5=1, and N6=1. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of three different tumor-selective targeting ligands to three different targeting receptors and two effector agents or where N1=3, and N2=0, and N3=0, and N4=2, and N5=0, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of three different tumor-selective targeting ligands to three different targeting receptors and two effector agents and two triggers, or where N1=3, and N2=0, and N3=0, and N4=2, and N5=2, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of three different tumor-selective targeting ligands to three different targeting receptors and two effector agents, and two triggers, and one masked intracellular transport ligand or where N1=3, and N2=1, and N3=0, and N4=2, and N5=2, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of three different tumor-selective targeting ligands to three different targeting receptors and two effector agents, and two triggers, and one masked intracellular transport ligand an intracellular trapping ligand or a masked intracellular trapping ligand, or where N1=3, and N2=1, and N3=0, and N4=2, and N5=2, and N6=1. In a preferred embodiment of this, the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of four different tumor-selective targeting ligands to four different target receptors and one effector agent or where N1=4, and N2=0, and N3=0, and N4=1, and N5=0, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of four different tumor-selective targeting ligands to four different target receptors and one effector agent and one trigger that increases the toxicity of the effector agent, or where N1=4, and N2=0, and N3=0, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of four different tumor-selective targeting ligands to four different target receptors and one effector agent and one trigger that increases the toxicity of the effector agent and one masked intracellular transporter ligand or where N1=4, and N2=1, and N3=0, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another embodiment the anti-cancer compound ET is comprised of four different tumor-selective targeting ligands to four different target receptors and one effector agent and one trigger that increases the toxicity of the effector agent and one masked intracellular transport ligand one intracellular trapping ligand or masked intracellular trapping ligand, or where N1=4, and N2=1, and N3=0, and N4=1, and N5=1, and N6=1. In a preferred embodiment of this the effector agent is a cytotoxic drug. In another embodiment of this, the effector agent is comprised of radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of four different tumor-selective targeting ligands to four different target receptors and one effector agent and one trigger that increases the toxicity of the effector agent and one masked intracellular transport ligand one intracellular trapping ligand or masked intracellular trapping ligand, and one trigger that decreases the toxicity of the effector agent or where N1=4, and N2=1, and N3=1, and N4=1, and N5=1, and N6=1. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of four different tumor-selective targeting ligands to four different target receptors and two effector agents or where N1=4, and N2=0, and N3=0, and N4=2, and N5=0, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of four different tumor-selective targeting ligands to four different target receptors and two effector agents and two triggers, or where N1=4, and N2=0, and N3=0, and N4=2, and N5=2, and N6=0.

In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of four different tumor-selective targeting ligands to four different target receptors and two effector agents, and two triggers, and one masked intracellular transport ligand or where N1=4, and N2=1, and N3=0, and N4=2, and N5=2, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of four different tumor-selective targeting ligands to four different target receptors and two effector agents, and two triggers, and one masked intracellular transport ligand an intracellular trapping ligand or a masked intracellular trapping ligand, or where N1=4, and N2=1, and N3=0, and N4=2, and N5=2, and N6=1. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with two targeting ligands wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound.

In another preferred embodiment the anti-cancer compound ET is comprised of a compound with two targeting ligands wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein ET is comprised of two different tumor-selective targeting ligands that bind to different targeting ligands and one effector agent or where N1=2, and N2=0, and N3=0, and N4=1, and N5=0, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with two targeting ligands that bind to different targeting ligands wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein ET is comprised of two different tumor-selective targeting ligands and one effector agent and one trigger that increases the toxicity of the effector agent or where N1=2, and N2=0, and N3=0, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with two targeting ligands wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein ET is comprised of two different tumor-selective targeting ligands that bind to different targeting ligands and one effector agent and one trigger and one masked intracellular transporter ligand or where N1=2, and N2=1, and N3=0, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with two targeting ligands wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein ET is comprised of two different tumor-selective targeting ligands that bind to different targeting ligands and one effector agent and one trigger that increases the toxicity of the effector agent and one masked intracellular transport ligand, and one intracellular trapping ligand or one masked intracellular trapping ligand or where N1=2, and N2=1, and N3=0, and N4=1, and N5=1, and N6=1. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with two targeting ligands wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein ET is comprised of two different tumor-selective targeting ligands that bind to different targeting ligands and one effector agent and one trigger that increases the toxicity of the effector agent, and one trigger that decreases the toxicity of the effector agent, and one masked intracellular transport ligand or where N1=2, and N2=1, and N3=1, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with two targeting ligands wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein ET is comprised of two different tumor-selective targeting ligands that bind to different targeting ligands and one effector agent and one trigger that increases the toxicity of the effector agent, and one trigger that decreases the toxicity of the effector agent, and one masked intracellular transport ligand one intracellular trapping ligand or masked intracellular trapping ligand or where N1=2, and N2=1, and N3=1, and N4=1, and N5=1, and N6=1. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with two targeting ligands wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein ET is comprised of two different tumor-selective targeting ligands that bind to different targeting ligands and two tumor-selective targeting ligands and two effector agents or where N1=2 and N4=2. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with three different targeting ligands that bind to three different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound.

In another preferred embodiment the anti-cancer compound ET is comprised of a compound with three different targeting ligands that bind to three different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with three different targeting ligands that bind to three different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of one effector agent or where N1=3, and N2=0, and N3=0, and N4=1, and N5=0, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with three different targeting ligands that bind to three different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of one effector agent and one trigger that increases the toxicity of the effector agent, or where N1=3, and N2=0, and N3=0, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with three different targeting ligands that bind to three different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of one effector agent and one trigger that increases the toxicity of the effector agent and one masked intracellular transporter ligand or where N1=3, and N2=1, and N3=0, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with three different targeting ligands that bind to three different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of one effector agent and one trigger that increases the toxicity of the effector agent and one masked intracellular transport ligand one intracellular trapping ligand or masked intracellular trapping ligand, or where N1=3, and N2=1, and N3=0, and N4=1, and N5=1, and N6=1. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with three different targeting ligands that bind to three different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of one effector agent and one trigger that increases the toxicity of the effector agent and one masked intracellular transport ligand one intracellular trapping ligand or masked intracellular trapping ligand, and one trigger that decreases the toxicity of the effector agent or where N1=3, and N2=1, and N3=1, and N4=1, and N5=1, and N6=1. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with three different targeting ligands that bind to three different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of two effector agents or where N1=3, and N2=0, and N3=0, and N4=2, and N5=0, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with three different targeting ligands that bind to three different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of two effector agents and two triggers, or where N1=3, and N2=0, and N3=0, and N4=2, and N5=2, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with three different targeting ligands that bind to three different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of two effector agents, and two triggers, and one masked intracellular transport ligand or where N1=3, and N2=1, and N3=0, and N4=2, and N5=2, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with three different targeting ligands that bind to three different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of two effector agents, and two triggers, and one masked intracellular transport ligand an intracellular trapping ligand or a masked intracellular trapping ligand, or where N1=3, and N2=1, and N3=0, and N4=2, and N5=2, and N6=1. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins. In another preferred embodiment the anti-cancer compound ET is comprised of a compound with four different targeting ligands that bind to four different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound. In another preferred embodiment the anti-cancer compound ET is comprised of a compound with four different targeting ligands that bind to four different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of four different tumor-selective targeting ligands and one effector agent or where N1=4, and N2=0, and N3=0, and N4=1, and N5=0, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with four different ligands that bind to four different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of four different tumor-selective targeting ligands and one effector agent and one trigger that increases the toxicity of the effector agent, or where N1=4, and N2=0, and N3=0, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor or multi-drug transporter proteins. In another embodiment of this, the effector agent is comprised of an inhibitor to nucleoside transporter proteins.

In another preferred embodiment the anti-cancer compound ET is comprised of a compound with four different targeting ligands that bind to four different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of four different tumor-selective targeting ligands and one effector agent and one trigger that increases the toxicity of the effector agent and one masked intracellular transporter ligand or where N1=4, and N2=1, and N3=0, and N4=1, and N5=1, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with four different targeting ligands that bind to four different target receptors and wherein at least one of the targeting ligands binds to a target receptor on the surface of the tumor cell or in the microenvironment of the tumor cell wherein the tumor has an increased a mount of that target receptor compared to a normal cell or a vital normal cell that binds to a second targeting ligand of the compound; and wherein the compound is comprised of four different tumor-selective targeting ligands and one effector agent and one trigger that increases the toxicity of the effector agent and one masked intracellular transport ligand one intracellular trapping ligand or masked intracellular trapping ligand, or where N1=4, and N2=1, and N3=0, and N4=1, and N5=1, and N6=1. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with four different targeting ligands that bind to four different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of four different tumor-selective targeting ligands and one effector agent and one trigger that increases the toxicity of the effector agent and one masked intracellular transport ligand one intracellular trapping ligand or masked intracellular trapping ligand, and one trigger that decreases the toxicity of the effector agent or where N1=4, and N2=1, and N3=1, and N4=1, and N5=1, and N6=1. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with four different targeting ligands that bind to four different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of four different tumor-selective targeting ligands and two effector agents or where N1=4, and N2=0, and N3=0, and N4=2, and N5=0, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with four different targeting ligands that bind to four different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of four different tumor-selective targeting ligands and two effector agents and two triggers, or where N1=4, and N2=0, and N3=0, and N4=2, and N5=2, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with four different targeting ligands that bind to four different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of four different tumor-selective targeting ligands and two effector agents, and two triggers, and one masked intracellular transport ligand or where N1=4, and N2=1, and N3=0, and N4=2, and N5=2, and N6=0. In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment the anti-cancer compound ET is comprised of a compound with four different targeting ligands that bind to four different target receptors and wherein at least one of the targeting ligands binds to a target receptor on the surface of the tumor cell or in the microenvironment of the tumor cell wherein the tumor has an increased amount of that target receptor compared to a normal cell or a vital normal cell that binds to a second targeting ligand of the compound; and wherein the compound is comprised of four different tumor-selective targeting ligands and two effector agents, and two triggers, and one masked intracellular transport ligand an intracellular trapping ligand or a masked intracellular trapping ligand, or where N1=4, and N2=1, and N3=0, and N4=2, and N5=2, and N6=1.

In a preferred embodiment of this the effector agent is a cytotoxic drug. In another embodiment of this, the effector agent is comprised of a radionuclide. In another embodiment of this, the effector agent is comprised of a drug that stimulates the immune system. In another embodiment of this, the effector agent is comprised of an effector agent that irreversibly chemically modifies one or more tumor components. In another embodiment of this, the effector agent is comprised of an inhibitor to multi-drug transporter proteins. In another embodiment of this, the effector agent is comprised of an inhibitor to nucleoside transporter proteins.

In another preferred embodiment, ET is an anti-cancer drug comprised of a compound with two or more targeting ligands that binds to a tumor cell with an affinity that is greater than a normal cell presenting a target receptor(s) that bind to the targeting ligands of said compound. In preferred embodiments the above mentioned binding affinity to the tumor cell is at least about 2-5 times greater, or at least about 5-10 times greater, or at least about 10-50 times greater, or at least about 50-500 times greater, or at least about 500-5,000 times greater, or at least about 5,000-50,000 times greater, or at least about 50,000-1,000,000 times greater or more than 1 million times greater than to a normal cell or to a vital normal cell. In a preferred embodiment the compound has three different targeting ligands. In another preferred embodiment the compound has 4 different targeting ligands.

In another preferred embodiment ET is an anti-cancer drug with binding affinity to tumor cells that is approximately the same as to populations of normal cells. However, said population of normal cells have decreased sensitivity to the toxic effects of the effector agent because said normal cells have decreased levels of an intracellular trapping receptor, or decreased sensitivity to the effector agent, or decreased levels of a specific protein necessary for neoantigen formation, or by virtue of said normal cells being located in the body at a site, such as the brain, where the drug ET cannot penetrate. In another preferred embodiment the anti-cancer drug ET is comprised of:

I. N1 targeting ligands, which can differ;

II. N2 masked intracellular transport ligands which can differ;

III. N3 triggers, which can differ, designated “detoxification triggers” wherein activation of the trigger decreases the toxicity of the drug;

IV. N4 effector agents which can differ;

V. N5 triggers which can differ, wherein activation of the trigger increases the toxicity of the drug;

VI. N6 intracellular trapping ligands or masked intracellular trapping ligands, which can differ;

and wherein:

N1=1, 2, 3, or 4, or about 4;

N2=0, 1, or 2, or about 2;

N3=0,1, or 2, or about 2;

N4=1, 2, or 3, or about 3;

N5=0,1, 2,or 3, or about 3;

N6=1, 2, or 3, or about 3;

And, wherein ET evokes a greater toxicity to a tumor cell compared to a non-tumor cell or a vital normal cell and wherein this increased antitumor selectivity is due to functional cooperation between the components of ET and not due to any single component of ET.

In a preferred embodiment of the invention and of the embodiments ET12.ET1, and ET12.ET2, and ET12.ET3, and ET12.ET3, and ET12.ET4, and ET12.ET5, and ET12.ET5 and ET12.ET6, and ET12.ET7, and ET12.ET8 and (ET12.ET8.X with X=1, 2, 3, 4 . . . 383) and ET12.ET9; and ET12.ET10, and ET12.ET8.X with X=1, 2, 3, 4, 5 . . . X);

the compound ET is comprised of an anti-cancer drug with at least one targeting ligand that binds to a target receptor selected from the following list:

1.) a cathepsin type protease

2.) a collagenase

3.) a gelatinase

4.) a matrix metalloproteinase

5.) a membrane type matrix metalloproteinase

6.) alpha v beta 3 integrin

7.) bombesin/gastrin releasing peptide receptors

8.) cathepsin B

9.) cathepsin D

10.) cathepsin K

11.) cathepsin L

12.) cathepsin O

13.) fibroblast activation protein

14.)folate binding receptors

15.) gastrin/cholecystokinin type B receptor

16.) glutamate carboxypeptidase II or (PSMA)

17.) guanidinobenzoatase

18.) laminin receptor

19.) matrilysin or

20.) matripase

21.) melanocyte stimulating hormone receptor

22.) nitrobenzylthioinosine-binding receptors

23.) norepenephrine transporters

24.) nucleoside transporter proteins

25.) peripheral benzodiazepam binding receptors

26.) plasmin

27.) seprase

28.) sigma receptors

29.) somatostatin receptors

30.) stromelysin 3

31.) trypsin

32.) urokinase

33.) MMP 1

34.) MMP 2

35.) MMP 3

36.) MMP 7

37.) MMP 9

38.) Membrane type matrix metalloproteinase I

39.) MMP 12

40.)MMP 13

In a preferred embodiment of the present invention ET is comprised of an anti-cancer drug with two targeting ligands for receptors that are increased on a tumor cell compared to a normal cell wherein at least one of the targeting ligands binds to a receptor selected from the list given above.

In a preferred embodiment ET is comprised of an anti-cancer drug with 2 targeting ligands that bind to receptors selected from the above list. In a preferred embodiment these receptors are the same. In a preferred embodiment these receptors are different and bind to different receptors.

In a preferred embodiment ET is comprised of an anti-cancer drug with 3 targeting ligands that bind to receptors selected from the above list. In a preferred embodiment these receptors are the same. In a preferred embodiment these receptors are different and bind to different receptors.

In a preferred embodiment ET is comprised of an anti-cancer drug with 4 targeting ligands that bind to receptors selected from the above list. In a preferred embodiment these receptors are the same. In a preferred embodiment these receptors are different and bind to different receptors.

In a preferred embodiment of the anti-cancer drug ET the targeting ligands are selected to bind to targeting receptors that are enriched on tumor cells or in the microenvironment of tumor cells.

Mechanism of Action

A preferred embodiment of the invention comprises an anti-cancer drug comprised of 2 to n targeting ligands designated as “A1”, and “A2”, . . . “An” that are connected by a linker designated as “L”, and wherein “An” refers to a targeting ligand that can bind to a targeting receptor designated “an” that is enriched on the surface of or in the microenvironment of the target and to which is also attached aone or more cytotoxic cytotoxic agents.

The targeting ligand-target receptor complex a1-A1-L-A2-a2 can be stabilized by the binding energy of both the A1-a1 and A2-a2 interactions which can result in extraordinary affinity of E-T to the target cell. By this mechanism super high affinity (essentially irreversible) targeting is possible provided that both A1-a1 and A2-a2 are high affinity bindings. Doubling the decrease in standard free energy for a reaction squares the equilibrium constant. Although entropic factors can intervene to preclude the addition of a second receptor site from actually doubling the standard free energy change, the impact on the equilibrium constant (binding affinity) can be enormous. Targeting affinity exceeding that seen with monoclonal antibodies can be achieved with low molecular weight compounds. An important consequence of this type of multivalent binding is multifactorial targeting. Super high affinity binding can occur only if the target cell has both targeting receptors a1 and a2. The higher the affinity the lower the drug concentration required to bind the drug to the target cell. Accordingly, at sufficiently low concentrations the drug can bind almost exclusively to target cells that jointly express both a1 and a2.

The relationship between increased binding affinity and multisite binding is a consequence of the most basic laws of thermodynamics and is exemplified by the properties of antibodies, peptabodies, certain drug dimers which display multisite binding affinity up to a million times greater than with single site binding. The following reference relates to this subject matter: Kaufman E. N.; Jain R. K., “Effect of Bivalent Interaction upon Apparent Antibody Affinity: Experimental Confirmation of Theory Using Fluorescence Photobleaching and Implications for Antibody Binding Assays,” Cancer Research, 52:4157-4167 (1992); Terskikh A. V., et al., “‘Peptabody’: A New Type of High Avidity Binding Protein,” Proc NatlAcad Sci USA, 94:1663-1668 (1997); Hubble J., “A Model of multivalent Ligand-receptor Equilibria which Explains the Effect of Multivalent Binding Inhibitors,” Molecular Immunology, 36:13-18 (1999); Page D.; René Roy, “Synthesis and Biological Properties of Mannosylated Starburst Poly(amidoamine) Dendrimers,” Bioconjugate Chem, 8:714-723 (1997); Calas M., et al., “Antimalarial Activity of Compounds Interfering with Plasmodium falciparum Phospholipid Metabolism: Comparison between Mono- and Bisquaternary Ammonium Salts,” J Med Chem, 43:505-516 (2000); Kramer R. H.; Karpen J. W., “Spanning Binding Sites on Allosteric Proteins with Polymer-linked Ligand Dimers,” Nature, 395:710-713 (1998); Fan E., et al., “High-Affinity Pentavalent Ligands of Escherichia coli Heat-Labile Enterotoxin by Modular Structure-Based Design,” J Am Chem Soc, 122:2663-2664 (2000); Blaustein R. O., et al., “Tethered Blockers as Molecular ‘Tape Measures’ for a Voltage-gated K+ Channel,” Nature Structure Biol, 7(4):309-311 (2000); Riley A. M.; Potter B. V. L., “Poly(ethylene glycol)-Linked Dimers of D-myo-inositol 1,4,5-trisphosphate,” Chem Commun, 983-984 (2000); Mammen M., et al., “Polyvalent Interactions in Biological Systems: Implications for Design and Use of Multivalent Ligands and Inhibitors,” Angew Chem Int Ed, 37:2754-2794 (1998); Johnson D. L., et al., “Amino-terminal Dimerization of an Erythropoietin Mimetic Peptide Results in Increased Erythropoietic Activity,” Chem Biol, 4:939-950 (1997), the contents of which are incorporated herein by reference in their entirety.

If A1 and A2 are identical and the target site a1 is present at sufficient density on the target cell then drugs incorporating this structure can induce crosslinking of the cell receptors. Many membrane associated proteins are highly mobile within the surface of the cell membrane. The binding energy of the drug to the cell can also be substantially increased which can translate into a markedly increased affinity and potency of targeting. If the affinity of A1 to its target site is high then the crosslinked form can be essentially irreversible. Crosslinking of the receptors can also enhance cellular uptake by triggering endocytosis. The following reference relates to this subject matter: York S. J. et al., “The Rate of Internalization of the Mannose 6-Phosphate/Insulin-like Growth Factor II Receptor is Enhanced by Multivalent Ligand Binding,” J Biol Chem, 274(2):1164-1171 (1999), the contents of which are incorporated herein by reference in their entirety.

The rate of crosslinking can be a function of the square of the receptor concentration. For example, if a tumor cell has 10 times more target sites al than normal cells then the tumor cell can form crosslinked receptors at a rate 100 times faster (to a first approximation) than the normal cells. A prerequisite for the successful application of this class of compounds is that the receptor density on the target cell be sufficiently high to allow crosslinking to occur at a meaningful rate. The linker length can be selected to optimize crosslinking capacity.

In the embodiment where A1 and A2 are different, the rate of crosslinking and essentially irreversible binding of the prodrug to the cell can be a function of the product of the concentration of the receptor target sites a1 and a2. For example, if a tumor cell has 10 times more a1 and 30 times more a2 than normal cells then the tumor cell can form crosslinked receptors at a rate approximately 300 times faster than the normal cells. If the product of the concentration is too low then the magnitude of the avidity enhancement can be minimal. Accordingly, if a1 is a target receptor, which is present only at very low concentrations, then a2 can be selected to be a target receptor, which is present at high concentrations.

The embodiment, in which a1 is enriched on the target cell and a2 is present on target and normal cells at equal concentrations, also has useful applications. For example, if a1 is a cell membrane protein, which is poorly internalized then a drug complex coupled to a1 can fail to enter the target cell. However, if a2 is a cell membrane protein that undergoes facile endocytosis then crosslinked complex can be transported into the cell with increased efficiency.

A2 can also serve to localize the drug to the cell membrane. For example, if A2 is a simple fatty acid it can partition into the cell membrane in a nonspecific fashion. Nonetheless, this can contribute significantly to the binding energy of the drug to the cell and markedly increase overall target cell affinity. The transfer of a fatty acid chain from solution to the lipid phase of the membrane is expected to be a much slower process then the binding of typical high affinty ligand-receptors that are often under diffusion control. Since the equilbrium constant is the ratio of the forward and backward reaction rates (rate of solvation/rate of desolvation), the rate at which the fatty acid group desolvates from the cell membrane can be even slower which can contribute to the retention of the targeted drug to the target cell. Accordingly, the use of a nonspecific group which binds with relatively low energy, and has minimal entropic requirements, in conjunction with a target selective high affinity ligand can markedly enhance targeting effectiveness. It can be noted that the drugs are designed for use in the nanomolar to picomolar range orders of magnitude below the critical micelle concentration.

In another preferred embodiment the drug has three target selective ligands A1, A2, and A3. Drugs of this type can bind with high affinity to target cells that express all three or any combination of two of the receptors (a1, a2, a3 or a1, a2, or a2, a3 or, a1, a3). The advantage of having three receptors is that loss of one receptor is unlikely to confer the tumor resistance to the drug.

In a preferred embodiment, A1 and A2 are selected so as to bind to target sites that are enriched on tumor cells compared to normal cells. For example, A1 or A2 can bind to a receptor, structural component, or enzyme located on the tumor cell surface or to an enzyme that binds to the cell surface.

A preferred embodiment of the invention and of embodiment ET8 has the structure shown below:

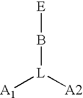

Wherein A1 and A2 are tumor-selective targeting ligands, and the L are linkers and B is a trigger that when activated frees the effector agent E from the remainder of the drug; and wherein E is a cytotoxin.

Drugs of this class feature two tumor specific high affinity binding ligands covalently coupled via a linker designed to allow both A1 and A2 to interact with receptors a1 and a2 on the tumor cell surface. A toxic moiety is coupled covalently to the linker via a functionality which has a trigger mechanism that when activated releases the toxin. The requirements for the trigger functionality differ depending upon the nature of the toxin to be delivered and the rate of cellular uptake of E-T. If the free toxin is readily internalized by the target cells then a trigger can be activated by extracellular or ultracellular enzymes or chemical processes. In a preferred embodiment, the trigger can be activated by an enzyme that is enriched in the tumor microenvironment. If the free toxin is poorly taken up by tumor cells, then a trigger that is preferentially activated inside cells can be used to free the drug intracellularly. This can be achieved by employing a trigger that is activated by intracellular enzymes. Alternatively, the trigger can be activated by extracelluar enzymes or by spontaneous chemical processes provided that a time delay mechanism is incorporated which allows sufficient time between trigger activation and toxin release for the drug ligand complex E-T to be internalized. Finally, in circumstances where the toxin is effective extracellullary (or intracellularly when still attached to the targeting ligands), the trigger can be omitted entirely.

In a preferred embodiment the trigger can be activated by an enzyme that is delivered to the target cell via independently selective mechanisms. There have been intense efforts towards the development of tumor-selective antibodies coupled to enzymes to selectively activate prodrugs. A significant limitation with Antibody Directed Enzyme Prodrug Therapy (ADEPT), and related approaches is the requirement that for the targeted enzyme to efficiently activate the prodrug, the prodrug can be given at a concentration near the Michaelis Menton constant (Km) for the enzyme substrate interaction which is generally micromolar. Since all drugs are expected to have multiple pathways of metabolism, prodrug activation by non-targeted enzyme mechanisms can result in dose limiting toxicity. The following reference relates to this subject matter: Bagshawe K. D., “ADEPT and Related Concepts,” Cell Biophys, 24-25:83-91 (1994); Syrigos K. N.; Epenetos A. A., “Antibody Directed Enzyme Prodrug Therapy (ADEPT): A Review of the Experimental and Clinical Considerations,” Anti-cancer Res, 19(1A):605-13 (1999); Bagshawe K. D., “Antibody-Directed Enzyme Prodrug Therapy for Cancer: Its Theoretical Basis and Application,” Mol Med Today, 1(9):424-31 (1995); Melton R. G.; Sherwood R. F., “Antibody-Enzyme Conjugates for Cancer Therapy,” J Nati Cancer Inst, 88(3-4):153-65 (1996); Stribbling S. M., et al., “Biodistribution of an Antibody-Enzyme Conjugate for Antibody-Directed Enzyme Prodrug Therapy in Nude Mice Bearing a Human Colon Adenocarcinoma Xenograft,” Cancer Chemother Pharmacol, 40(4):277-84 (1997); Bagshawe K. D., et al., “Developments with Targeted Enzymes in Cancer Therapy,” Curr Opin Immunol, 11 (5):579-83 (1999); Sharma S. K., et al., “Human Immune Response to Monoclonal Antibody-Enzyme Conjugates in ADEPT Pilot Clinical Trial,” Cell Biophys, 21(1-3):109-20 (1992); Dowell R. I., et al., “New Mustard Prodrugs for Antibody-Directed Enzyme Prodrug Therapy: Alternatives to the Amide Link,” J Med Chem, 39(5):1100-5 (1996); Connors T. A.; Knox R. J., “Prodrugs in Cancer Chemotherapy,” Stem Cells (Dayt), 13(5):501-1 (1995); Springer C. J., et al., “Prodrugs of Thymidylate Synthase Inhibitors: Potential for Antibody Directed Enzyme Prodrug Therapy (ADEPT),” Anti-cancer Drug Des, 11(8):625-36 (1996); Wallace P. M.; Senter P. D., “Selective Activation of Anti-cancer Prodrugs by Monoclonal Antibody- Enzyme Conjugates,” Methods Find Exp Clin Pharmacol, 16(7):505-12 (1994); Denny W. A.; Wilson W. R., “The Design of Selectively-Activated Anti-Cancer Prodrugs for Use in Antibody-Directed And Gene-Directed Enzyme-Prodrug Therapies,” J Pharm Pharmacol, 50(4):387-94 (1998); Senter P. D.; Svensson H. P., “A Summary of Monoclonal Antibody-Enzyme/Prodrug,” Adv Drug Delivery Rev, 22:341-349 (1996); Roger G. Melton, “Preparation and Purification of Antibody-Enzyme Conjugates for Therapeutic Applications,” Adv Drug Delivery Rev, 22:289-301 (1996); Roger F. Sherwood, “Advanced Drug Delivery Reviews: Enzyme Prodrug Therapy,” Adv Drug Delivery Rev, 22:269-288 (1996); Niculescu-Duvaz I.; Springer C. J., “Antibody-Directed Enzyme Prodrug Therapy (ADEPT): A Review,” Adv Drug Delivery Rev, 26:151-172 (1997); Ravi V. J. Chari, “Targeted Delivery of Chemotherapeutics: Tumor-Activated Prodrug Therapy,” Adv Drug Delivery Rev, 31:89-104 (1998); U.S. Pat. No. 4,975,278, Dec. 4, 1990, Senter, et al., “Antibody-enzyme Conjugates in Combination with Prodrugs for the Delivery of Cytotoxic Agents to Tumor Cells”, the contents of which are incorporated herein by reference in their entirety.

Drugs embodied by the present invention can preferably be used at extremely low concentrations in vivo, generally in the nanomolar to picomolar range or lower. At these concentrations the fate of the drug can be defined by high affinity targeting interactions under perhaps nonequilibrium conditions. Typically metabolic enzymes function by forming an enzyme substrate complex that is transformed into the products. In general, the Km for enzymes is in the micromolar range. Accordingly, drug metabolism can predominantly occur at the sites where the drug is trapped by the high affinity binding, provided that the drug has a sufficiently long half-life to allow distribution to the target site. If the drug E-T is selectively localized to the tumor surface and the triggering enzyme is also selectively localized to the tumor surface then greatly enhanced antitumor selectivity can result.

If the free toxin is poorly internalized by cells, then the extracellular liberation of the toxin from E-T can functionally detoxify the drug. In a preferred embodiment, of the present invention applied to this circumstance the trigger can be activated by an enzyme which is enriched in non-tumor cells where dose limiting toxicity takes place. In an even more preferred embodiment of the invention the (detoxifying) trigger can be activated by an enzyme that is selectively delivered to non-tumor cells. For example, the detoxifying trigger can be activated by an enzyme that is coupled to an antibody selective for bone marrow stem cells. This can allow for the selective detoxification of the drug by bone marrow stem cells. Currently, the sparing of bone marrow stem cell toxicity is accomplished by the use of bone marrow transplantation, which is a risky and costly one time procedure. There is a very significant practical advantage to employing a prodrug of the present class along with a detoxifying enzyme that is selectively targeted to vital normal cells. Targeting of normal cells is an easier proposition than targeting tumor cells. The blood supply is generally superior in normal tissues. Most importantly, to achieve a protective effect it can be sufficient to deliver antibody-enzyme conjugate to a minority of the normal bone marrow stem cells. In contrast, to achieve a therapeutic effect (3 log reduction in tumor burden) by targeting the tumor cells it is necessay to deliver antibody enzyme complex to 99.9% of the tumor cells.

The scope of the present invention includes a method of sparing vital normal cells of drug toxicity by targeting, to the normal cells, an enzyme that activates a detoxification trigger on the administered targeted drug that detoxifies the drug.

The scope of the present invention includes the set of a targeted drug with a detoxfication trigger and a targeted enzyme that can activate the detoxification trigger and detoxify or markedly lower the toxicity of the drug.

The scope of the present invention includes a drug that has a detoxification trigger that when activated functionally detoxifies or lowers the toxicity of the drug by interfering with cellular uptake.

In another preferred embodiment of the present invention, E-T comprises the following structure:

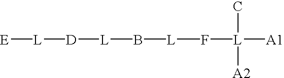

wherein A1 and A2 are targeting ligands; B is a trigger that upon activation liberates the effector agent portion of the molecule from the targeting ligands; C is a masked intracellular transport ligand; D is an intracellular trapping ligand or masked intracellular trapping ligand; E is an effector agent; and F is a detoxification trigger that when activated decreases the toxicity or effector activity by interfering with cellular uptake of the effector agent into the cell.

The drug can bind with very high affinity to targeted tumor cells via receptors a1 and a2. At the tumor cells surface either spontaneous chemical processes or enyzymatic processes can trigger the unmasking of the intracellular transporter ligand that is comprised of a ligand that binds to a cellular receptor that then actively transports the complex into the cell. Trigger B can either be activated by intracellular enzymes or be activated extracellularly with a delay mechanism that allows sufficient time for the complex to be transported into the cell prior to the release of the toxin. The trigger, which unmasks the intracellular transporter ligand, can be activated by enzymes that are enriched in the tumor microenvironment, or by ubiquitous enzymes, or by spontaneous chemical processes. If the unmasking trigger can be activated by a ubiquitous enzyme such as esterase, then it is desirable to incorporate a time delay mechanism. The time delay mechanism can serve to allow time for the targeting receptors rather then the intracellular transport functionality to define the specificity of drug distribution. A time delay mechanism can be made having a triggering event such as the enzymatic cleavage of an ester that initiates a second chemical reaction that proceeds at a rate with the desired half-life. Triggers are described in detail in a latter section.

In one embodiment of the invention, the detoxifying trigger can be activated by an enzyme that is selectively delivered to non-tumor cells. Complementary to this, is this case in which the trigger that unmasks the transport ligand can be activated by an enzyme that is selectively and independently targeted to the tumor cells.

Multifunctional drug delivery vehicles with both toxifying and detoxifying triggers can have the ability to be either toxic or nontoxic to cells depending upon relative rates of activation of the respective trigger functionalities. The drugs have a logic circuit with decision-making ability. The input corresponds to the levels of enzyme activity available to activate the toxifying and detoxifying triggers respectively. The output is increased or decreased drug toxicity for the potential target cell. Glazier previously disclosed a class of anti-cancer drugs that have toxification and detoxification functionalities.

The following reference relates to this subject matter: U.S. Pat. No. 5,274,162, Dec. 28, 1993, Glazier, “Antineoplastic Drugs with Bipolar Toxification/Detoxification Functionalities.”; U.S. Pat. No. 5,659,061, Aug. 19, 1997, Glazier, “Tumor Protease Activated Prodrugs of Phosphoramide Mustard Analogs with Toxification and Detoxification Functionalities”, the contents of which are incorporated herein by reference in their entirety. However, the previously disclosed Antineoplastic Drugs with Bipolar Toxification/Detoxification Functionalities lacked targeting ligands, would need to be used at relatively high doses, and could potentially undergo substantial non-target site metabolism. The present invention can allow for very high affinty multifactorial drug targeting. In preferred embodiments the present drugs can be employed at ultra-low doses under conditions in which drug metabolism (activation of triggers) can be defined by the tumor microenvironment.

The scope of the present invention includes, the class of drugs E-T, wherein the drug binds to the target cell and exerts the biological effector activity of E depending upon the input received by triggers that turn on (or increase) or turn off (or decrease) the biological effector activity of E. This class of drugs enables multifactorial targeting in which the factors or properties that define targeting selectivity and biological activity include both the targeting receptors and triggering factors tr1 . . . trn. The designation “trn” is used to refer to enzymes or biomolecules or other factors that activate a particular trigger referred to as “trigger N” or “TRN.”

The scope of the present invention also encompasses the method comprising the following steps:

1.) The administration of one or more targeted drugs E-T that has one or more triggers TR1 . . . TRN that when activated by the triggering factors tr1 . . . trn undergoes either an increase or decrease in drug effector activity.

2.) The administration of one or more compounds (Txn-trn) comprised of targeting groups (Txn) linked to a triggering factor (trn), such that the targeting group delivers the triggering factors to selected population of cells (Pxn); and thereby modulates the biological activity of the drug(s) ET at the population of cells Pxn.

This technology can allow an enhancement of tumor selectivity. Vital normal cell populations can be targeted with triggering factors trn that activate the detoxification trigger and decrease the toxicity of the drug E-T. While tumor cells can be targeted with triggering factors that activate toxifying triggers and thereby enhance the toxicity of the drug E-T.

The triggering factor trn can be a wide range of enzymes that utilize a component of the trigger as a substrate and thereby activate the trigger functionality. The targeting group Txn can be any group or set of groups linked together that bind to the desired population of cells Pxn. Depending upon the context, the targeting group Txn can be selective for tumor cells or for normal cells. A large number of targeting groups selective for tumor cells are described in other sections. Suitable targeting groups include ligands that bind to receptors that are enriched on tumor cells, monoclonal antibodies, monoclonal antibody analogs, Fab portions or an antibody or monoclonal antibody, growth factor or any other structure which binds selectively to the target cell.

When targeting Txn-trn to normal cells the Txn can be selected to bind to receptors that are enriched on vital normal cells relative to tumor cells. For example, Txn can be a monoclonal antibody specific for the CD34 antigen, which is present on the surface of vital bone marrow stem cells but absent from most tumors. The complex Txn-trn could then be used to selectively detoxify the drug E-T on CD34 + bone marrow stem cells. The following reference relates to this subject matter: Civin C. I, et al., “Highly Purified CD34-Positive Cells Reconstitute Hematopoiesis,” J Clin Oncol, 14(8):2224-33 (1996), the contents of which are incorporated herein by reference in their entirety.

Many malignancies are characterized by the loss of critical membrane proteins. The present method allows the loss of one or more of these proteins from tumor cells to be a factor in defining the domain of tumor targeting. In a preferred embodiment, Txn-trn is selected such that Txn binds to a protein or factor that is lost or under-expressed on the surface of tumor cells and trn is comprised of an enzyme that activates a detoxification trigger on the drug E-T. In preferred embodiments, Txn is a monoclonal antibody or monoclonal antibody analog which binds to one of the following membrane associated proteins which is under-expressed in various human cancers: E-cadherin; Transforming growth factor beta receptors; Syndecan-1; Galectin -3; Deleted in colorectal cancer (DCC); Epil or Epitheal Protein Lost in Neoplasm; KAI protein; Connexin 43; H-cadherin; CD38; VLA-2 collagen receptor; P-cadherin; Luminal epithelial antigen (LEA135); Maspin; Mel-Cam; Billiary glycoprotein; Epithelial cell adhesion molecule C-CAM; Beta 4 integrin subunit; and Hemidesmosomal proteins.

Masked Intracellular Transport Ligands

Intracellular delivery is essential for the activity of many drugs. A general method to deliver drugs into cells is to couple the drugs to a ligand such as folic acid, which is taken up by cells via receptor mediated endocytosis. The following reference relates to this subject matter: U.S. Pat. No. 5,688,488, Nov. 18, 1997, Low, et al., “Composition and Method for Tumor Imaging.”; U.S. Pat. No. 5,416,016, May 16, 1995, Low, et al., “Method for Enhancing Transmembrane Transport of Exogenous Molecules.”, the contents of which are incorporated herein by reference in their entirety.

However, the use of an intracellular transport ligand such as folic acid can often define targeting selectivity to the benefit or the detriment of the therapy. If the intracellular transport ligand were simply folic acid then the spectrum of drug distribution and targeting would be significantly defined by the distribution of folate receptors in the body. Folate targeted moieties end up largely in the kidney, which is often undesirable. The following reference relates to this subject matter: Wang S., et al., “Design and Synthesis of [111In]DTPA-Folate for use as a Tumor-Targeted Radiopharmaceutical,” Bioconjug Chem, 8(5):673-9 (1997), the contents of which is incorporated herein by reference in its entirety. The properties of a complex of the protein pro-urokinase and saporin serves to illustrate how targeting and internalization can be mechanistically distinct. This complex binds to the urokinase receptor of tumor cells and is internalized following binding of the saporin to the low-density lipoprotein transport receptor. This example does not involve a masked intracellular transport ligand. The following reference relates to this subject matter: Ippoliti R., et al., “Endocytosis of a Chimera between Human Pro-Urokinase and the Plant Toxin Saporin: An Unusual Internalization Mechanism,” FASEB, 14(10):1335-1344 (2000), the contents of which is incorporated herein by reference in its entirety.

A compound ET, further comprising a masked intracellular transporter ligand provides a general solution to the problem of efficient intracellular drug transport while retaining targeting selectivity due to the targeting ligands. A masked intracellular transporter ligand is comprised of a group which when unmasked is able to bind to cellular receptors that transport bound ligands into the cell. The current invention allows targeting to be defined by the targeting ligands. A second major advantage is that the cell associated target receptors that provide targeting specificity need not possess the property of being able to transport the targeted drug into the cells. Finally, as discussed below, the masked intracellular transported ligand provides a means by which to provide a simultaneous plurality of intracellular transport mechanisms that can decrease the development of drug resistance.

A variety of masked transporter ligands can be employed. Preferably the following factors are considered individually or in combination in selecting the masked ligand:

1.) When unmasked the group can bind with sufficient affinity to a structure on the target cell, which can activate transport into the cell;

2.) The group has a chemical moiety which can be modified in a reversible manner such that the modification impairs the ability of the group to bind productively to the cellular transport mechanism (ie., a group that allows for masking);

3.) The masked transporter group can be capable of being unmasked by interaction with an enzyme, metabolite, or by a spontaneous chemical process; and

4.) The unmasked intracellular transporter group can bind to a protein or other factor that also binds to a cell membrane receptor and activates intracellular transport of bound ligands.

In a preferred embodiment, the masked intracellular transporter ligand is a folic acid derivative coupled via one of its carboxylate groups, preferably the gamma carboxylate group, through a linker to the rest of the drug, wherein the folic acid is substituted in a bioreversible manner such that binding of the derivative to the folate receptors is impaired in a bioreversible manner. Preferred sites of derivatization are nitrogen 10 or at the alpha carboxy group. A preferred embodiment comprises substitution at the N10 position of the folic acid by a bioreversible amino protecting group referred to as a “trigger” that can be modified in vivo and which, upon this modification referred to as “trigger activation”, unmasks the amino group. Another preferred embodiment comprises folic acid substituted at the alpha carboxy group to yield an ester or amide. These are illustrated below:

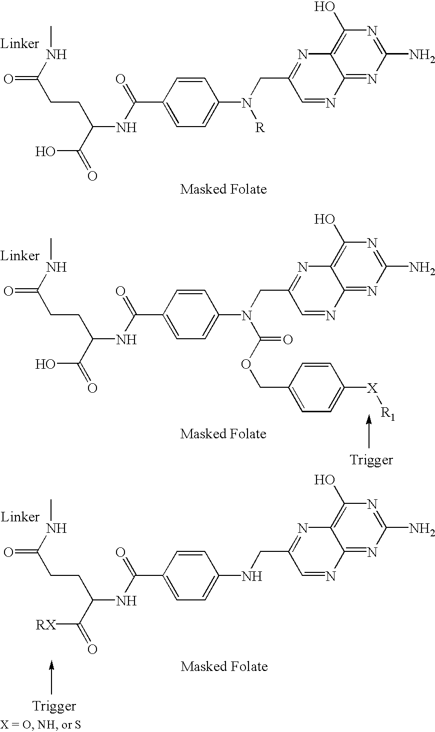

wherein R is a trigger group and R1 is a bioreversible protecting group for —X—H, and wherein X is 0, NH, or S. Preferred triggers and preferred embodiments of R1 are described in the trigger section of this document. Cleavage of the trigger can unmask the folate and initiate the process of active cell uptake. A wide variety of triggers can be employed including esters, phosphoesters, phosphodiesters, amides, substituted disulfides, oligopeptides, and glycosides. In principle, any functionality suited for use in the ADEPT approach as a trigger could be employed along with an appropriately selected target enzyme that cleaves that trigger. The trigger can be activated by tumor-selective proteases. A description of triggers of this type can be found in: U.S. Pat. No. 5,659,061, Aug. 19, 1997, Glazier A., “Tumor Protease Activated Prodrugs of Phosphoramide Mustard Analogs with Toxification and Detoxification Functionalities”, the contents of which is incorporated herein by reference in its entirety.

In a preferred embodiment, a clock-like time delay trigger is employed to unmask the intracellular transport ligand. Triggers of this type can allow the drug to have time to bind to the tumor prior to unmasking of the intracellular transport ligand. A variety of clock-like time delay triggers are described in the trigger section of the present invention.

In a preferred embodiment of the present invention the masked intracellular transporter ligand comprises biotin that is chemically modified in such a manner as to interfere with receptor binding in a bioreversible manner. Biotin can be linked to the remainder of the drug via its carboxylate group and can retain binding affinity to biotin receptors. A preferred embodiment comrpises biotin with bioreversible substitution of one or more of the ureido amidic protons as illustrated below:

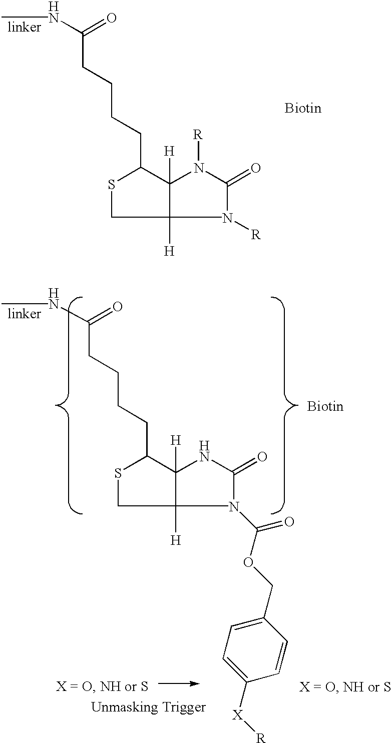

X and R can be groups as described previously for the masked folate trigger. Drugs of this class (with a masked biotin receptor) can be administered in conjunction with one or more transporter moieties to which is coupled a biotin binding factor such as avidin or streptavidin. The transporter moieties are selected such, they bind to receptors on the tumor cell surface and are internalized. Binding of the unmasked biotin to the administered avidin-transporter moiety can transport the drug complex into the tumor cell. The avidin-transporter moiety can be tumor-selective or non-selective without specificity for tumor cells. Its role is to efficiently deliver the drug already targeted and located on the tumor cell surface into the cell. It is preferred to administer the drug first, allow time for the tumor localization to occur and then to administer the avidin-transporter moiety. Although it can be pointed out that high affinity between the drug and the avidin-transporter can only occur after the biotin is unmasked. The avidin-transporter can be given intravenously at a sufficiently high dose to allow contact with the tumor cells. It is preferable to use simultaneously at least two different types of avidin-transporters to avoid the selection of tumor drug resistance based on lack of binding or impaired internalization of one particular type of avidin-transporter.

Some considerations for the avidin-transporter moiety are as follows:

1.) Avidin can be coupled to the transporter function in a fashion that does not impede high affinity biotin binding;

2.) The transporter function can bind to the target cells and be internalized; and

3.) The avidin-transporter can be of low toxicity.

Any protein, hormone, lipid, nutrient, or substance, which is internalized by cells by efficient endocytotic proceess, that can be coupled to a biotin-binding moiety such as avidin can be employed. Preferred transporter moieties include: transferrin, alpha 2 macroglobulin, insulin, folic acid, and epidermal growth factor. Monoclonal antibodies against receptors or occupied receptor complexes known to undergo endocytosis can also be used. Techniques for coupling biotin-binding factors such as avidin to other moieties are well known. The following reference relates to this subject matter: Mukherjee S., et al., “Endocytosis”, Physiological Reviews,77(3):759-803 (1997); Hanover John A.; Dickson Robert B. (1985) Transferrin: Receptor-Mediated Endocytosis and Iron Delivery. in “Endocytosis” (I. Pastan and M. Caningham, eds.), pp.131-161. Plenum Press, New York; Hermanson Greg T. (1996) “Bioconjugate Techniques.” Academic Press, Inc., the contents of which are incorporated herein by reference in their entirety.

The scope of the present invention includes compounds comprised of one or more masked intracellular transport ligands and the method of delivering drugs or other effector molecules into cells by contacting the cells with a compound that has one or more masked intracellular transport ligands.

The scope of the present invention also includes the method of delivering drugs and effector molecules into cells that comprises contacting the cells with a targeted drug ET and also contacting the cells with one or more targeted transport moiety that facilitates drug transport into the cell. In a preferred embodiment the drug ET and targeted transport moiety are each targeted to different targets present on the target cells. In a preferred embodiment the target is a tumor cell. Preferred tumor-selective targets are described throughout this document.

The scope of the present invention includes a preferred embodiment that comprises a method of delivering a targeted drug or effector molecule into cells by multiple independent non-target endocytotic receptors. This method can be useful to circumvent drug resistance due to the loss of a single intracellular transport or endocytotic receptor.

Another preferred embodiment of the present invention comprises the following structure:

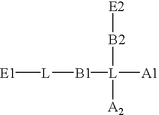

wherein A1 and A2 are targeting ligands; B1 and B2 are triggers that upon activation liberate the effector agent portion of the molecule from the targeting ligands and E is an effector agent. This embodiment incorporates, in addition to the features discussed previously, an effector mechanism comprised of two different cytotoxic agents, which can be released by two different triggering mechanisms. This feature can markedly decrease the rate at which tumor resistance develops to the drugs without significantly increasing overall drug toxicity. In addition, this can allow the joint delivery of two drugs that exhibit synergistic toxicity. In the preferred embodiment, the toxins are selected such that resistance to each is mediated by independent mechanisms. For example, if tumor resistance to one of the toxins is mediated by MDR1 gene product then ideally the second toxin can retain activity in cells expressing this phenotype. The following reference relates to this subject matter: Gottesman Michael M., “How Cancer Cells Evade Chemotherapy” Sixteenth Richard and Hinda Rosenthal Foundation Award Lecture”, Cancer Research, 53:747-754 (1993), the contents of which is incorporated herein by reference in its entirety.

A preferred embodiment of the present invention is the anti-cancer compound ET comprised both of a cytotoxic moiety(s) and an inhibitor to multi-drug resistance mechanisms such as MDRI P-glycoprotein. This can allow major mechanisms of tumor drug resistance to be overcome at a target specific level without increasing total systemic toxicity. The emergence of tumor resistance to a broad range of unrelated antineoplastic drugs by increased expression of the multi-drug transporter P-glycoprotein, which actively transports the drugs out of the tumor cells, is a major and fundamental limitation in cancer treatment. There have been extensive efforts towards the development of inhibitors to MDR P-glycoprotein. Clinical trials to date have been unsuccessful and complicated by systemic toxicity. The present invention can allow for the selective delivery of the multi-drug resistance inhibitors to tumor cells concurrently with the selective delivery of the anti-cancer drugs.

Targeting Specificity

The present invention can be used to target drugs to essentially any type of cell, cell population, tissue, or tissue type. The targeting specificity or targeting domain of multifunctional drug delivery vehicles can be defined as the populations of cells that are subjected to the effector action of the drug. The targeting domain of multifunctional drug delivery vehicles is a multifactorial or multivariable function in which the variables are targeting ligands specificity, specificity of triggers, and nature of the effector agent ultimately delivered. It is the interaction between these variables that ultimately defines the targeting domain and can allow exquisitely specific tumor targeting despite the fact that no single factor is unique to tumors.

The initial targeting specificity of the drugs can be defined by the combined high affinity interactions of the drug targeting ligands A1, . . . An with the target cell associated receptors a1 . . . an. The drugs are to be administered at a dose sufficient to bind an effective quantity to the targeted cell population or at a dose sufficient to evoke the desired therapeutic activity. For some of the preferred embodiments of the present invention the concentration range can generally be in the nanamolar to picomolar range or lower. The use of excessive concentrations can allow secondary non-targeting factors to dominate the pattern of drug distribution and metabolism with a potential reduction in the targeting selectivity and therapeutic index. The present class of drugs can be orders of magnitude more potent for targeted cells than the non-target toxin due to the high receptor mediated affinity of the drug to the targeted cells.

The targeting ligands A1, . . . An can be selected to bind a large variety of receptors a1, . . . an which are present at increased amounts on the surface of tumor cells compared to vital normal cells. The terms “target selective” and “tumor-selective” are used in a functional sense in this patent application. Absolute selectivity is elusive. Drugs always have some form of dose limiting toxicity that restricts the therapeutic index. A target can be considered tumor-selective if it is enriched on tumor cells compared to vital normal cells in the tissue that ordinarily suffers dose limiting toxicity. For example, if the dose limiting toxicity of the parent drug being delivered is bone marrow toxicity then a receptor or enzyme enriched on tumor cells compared to bone marrow stem cells would be a suitable “tumor-selective” target even if this target is not unique to tumor cells. Normal enzymes or receptors in abnormal locations can also function as tumor-selective targets and are a biochemical manifestation of metastasis. For example, if an enzyme is ordinarily confined to the luminal surface of the gastrointestinal tract the presence of that enzyme on malignant cells metastatic to the liver can be used for selective targeting. This can be accomplished by employing a drug that is given intravenously and fails to penetrate to the luminal surface of the GI tract. (Alternatively, the target sites on the normal GI cells can be blocked by an orally nonabsorbable inhibitor to the receptor or enzyme.) Useful tumor-selective targets can also be receptors or enzymes that are present on both malignant cells and normal cells provided that the targeted normal cells are not vital for life. Normal enzymes that are present intracellularly in normal cells but released or activated extracellularly in the tumor microenvironment can also be used for selective targeting provided that the drug is designed to remain in the extracellular space.

The targeted cell receptors can be any chemical moiety that is enriched on the target cells relative to the cell populations which one desires not to target. With the advent of combinatorial chemistry, and high throughput automated screening it is now possible to select high affinity ligands that can bind to essentially any biological receptor. The following reference relates to this subject matter: Wilson, Stephen R.; Czarnik, Anthony W.(eds.), “Combinatorial Chemistry; Synthesis and Application.” John Wiley & Sons, Inc., the contents of which is incorporated herein by reference in its entirety.

The steps in this process are well known to one skilled in the arts and include:

1.) Coupling a large library of potential receptor binding ligands to a linker and reporter functionality such as a fluorescent group, an enzyme, or a group such as biotin which can be readily detected;

2.) Coupling the receptor moiety to a solid phase;

3.) Incubating the receptor ligand-detector molecules with the receptor;

4.) Washing to remove unbound ligand; and

5.) Assaying for the reporter functionality bound to the receptor to identify high afffinity binding ligands.

For example, one can couple a fluorescent derivative via a linker to a library of millions of compounds and screen potential ligands for binding affinity to the desired receptor using a fluorescent based binding assay.

The hallmark of malignancy is uncontrolled cell proliferation and tissue invasion. Neither the processes of cell replication nor the enzymology of tissue invasion (remodeling) are by themselves uniquely diagnostic of malignancy. But jointly, these processes likely can provide highly selective criteria to define effective targeting for the treatment of malignancy. The current class of multifunctional anti-cancer drugs provides the opportunity to have anti-cancer agents that are targeted simultaneously and jointly to both the proliferative and the invasive character of malignant cells.

Antineoplastic agents directed against cell replication are well-known and typified by anti-cancer drugs such as alkylating agents, topoisomerase inhibitors, DNA antimetabolites, DNA polymerase inhibitors, and antimitotic agents. Targeting such drugs to cells that express the property of tissue invasiveness can significantly increase antitumor selectivity. Since the biochemical expression of tissue invasiveness is an essential component of malignancy, the development of tumor resistance by loss of these properties can be incompatable with persistence of the malignant phenotype. It is precisely for this reason that cytotoxic targeting towards the coupled expression of invasiveness and proliferation is so compelling. It is also important to recognize that tumors are composed of a heterogenous population with the most invasive and malignant cells defining the ultimate clinical outcome.

Many targeting receptors and receptor combinations are unrelated to cell survival or the processes of malignancy. Resistance to drugs directed towards these targeting features is predictable and expected. However, even if only a two log reduction in tumor burden is obtained prior to the development of resistance to the specific targeted drug by loss of the nonessential target receptor sites by the tumor cells, the net result is useful towards the overall goal of achieving sufficient log reductions of tumor burden to completely eliminate the disease.

There are five general classes of receptors which can be employed as “tumor-selective targets”:

1.) Enzymes and factors that are related to the biochemical manifestations of uncontrolled cell growth. Some examples include: autocrine growth factors, and abnormal receptor tyrosine kinases;

2.) Enzymes and molecules that are expressed by the tumor cells or in the microenvironment of tumor cells that are involved in the mechanism of tissue invasion. Some examples include collagenases, plasmin, urokinases, metalloproteinases, cathepsins, heparanase;

3.) Normal enzymes and receptors present in abnormal locations in association with tumor cells. Examples include: trypsin in ovarian cancer, sucrase-isomaltase in colon adenocarcinoma, pepsin in breast adenocarcinoma, and dipeptide transporter (PEPT1) colon adenocarcinoma;

4.) Normal enzymes and molecules associated with both tumor cells and normal tissue provided that the normal tissue is not vital to life or not sensitive to the delivered anti-cancer drug. Examples include: prostatic membrane surface antigen, prostatic specific antigen, hepsin in ovarian cancer, and neutral endopeptidase in leukemia; and

5.) Receptors unique to tumor cells, such as tumor specific antigens.

Suitable receptor targets include enzymes that are membrane associated with the target cell or which bind to receptors on the target cell, structural components of the target cell, or hormone receptors on the target cell. It is important to emphasize the point that targets, which individually cannot provide sufficient specificity in combination with the multifunctionality of the present invention, can provide useful targeting selectivity and in preferred cases can provide excellent target specificity. Targets also can be localized to the microenvironment of tumors. This is discussed in more detail in the section on targeted immunotherapy.

Targeting ligands can also bind to intracellular receptors that are enriched in target cells. For most anti-cancer drugs the biological activity is dependent upon intracellular concentration that is a function of the relative rates of drug influx and drug efflux. Many anti-cancer drugs are actively pumped out of cells by p-glycoprotein and related proteins. This is a major mechanism of tumor resistance to antineoplastic drugs. Intracellular targeting ligands that bind to intracellular receptors that are enriched in target cells can contribute to drug selectivity by trapping drug selectively in target cells. A variety of specific and non-specific intracellular trapping ligands are described elsewhere in this patent.

Preferred embodiments (embodiments TF#.X, wherein X is the number given below) include the anti-cancer compounds ET comprised of targeting ligands, triggers, and effector agents that are selective for combinations of the following factors or targeting properties:

1) 5′nucleotidase

2) 5-aminoimidazole-4-carboxamide ribonucleotide transferase

3) a cathepsin type protease

4) a collagenase

5) a gelatinase

6) a matrix metalloproteinase

7) a membrane type matrix metalloproteinase

8) acid phosphatase

9) activated Factor X

10) adenine phosphoribosyltransferase

11) alkaline phosphatase

12) alpha v beta 3 integrin

13) amino-peptidase N

14) androgen receptor

15) aspartate transcarbamylase

16) basic fibroblast growth factors and their receptors

17) bombesin /gastrin releasing peptide receptors

18) carbamoyl phosphate synthetase

19) carboxypeptidase M

20) cathepsin B

21) cathepsin D

22) cathepsin K

23) cathepsin L

24) cathepsin O

25) CD44

26) CXCR4 receptor

27) deoxycytidine kinase

28) deoxyguanosine kinase

29) dihydrofolate reductase

30) dihydroorotate dehydrogenase

31) dipeptidyl peptidase IV

32) emmprin

33) epidermal growth factor receptors and related proteins

34) epidermal growth factors

35) estrogen receptor

36) Fas ligand

37) fibroblast activation protein

38) folate binding receptors

39) galactosyltransferase

40) gamma-glutamyl transpeptidase

41) gastrin/cholecystokinin type B receptor

42) GDP-L-fucose:beta-D-galactoside alpha-2-L-fucosyltransferase

43) glutamate carboxypeptidase II or Prostate-specific membrane antigen

44) glutathione S-transferase

45) glycinamide ribonucleotide transformylase

46) gonadotropin releasing hormone receptor

47) GPIIb/IIIa fibrinogen receptor

48) guanidinobenzoatase

49) heparanase

50) hepsin

51) human glandular kallikrein 2

52) compounds made reactive or modified in vivo as the result of hypoxia

53) hypoxanthine-guanine phosphoribosyltransferase

54) inosine 5′monophosphate dehydrogenase

55) insulin-like growth factor receptors

56) insulin-like growth factors

57) laminin receptor

58) leutinizing hormone releasing receptor

59) matrilysin

60) matripase

61) melanocyte stimulating hormone receptor

62) mitogen activated protein kinase

63) multi-drug resistance protein

64) nerve growth factors and their receptors

65) neuroleukin/ phosphohexose isomerase lautocrine motility factor

66) neuropeptide Y receptors

67) neutral endopeptidase

68) nitrobenzylthioinosine-binding receptors (nucleoside transporter)

69) norepenephrine transporters

70) nucleoside transporter proteins

71) opioid receptors

72) orotidine-5′-phosphate decarboxylase

73) oxytocin receptor

74) p53 antigen

75) patelet derived growth factor receptor

76) pepsin c

77) peripheral benzodiazepam binding receptors

78) p-glycoprotein

79) phospatidylinositol 3-kinase

80) placental alkaline phosphatase

81) plasmin

82) platelet-derived growth factors and their receptors

83) polyamine transporters

84) porphyrin receptors

85) progesterone receptors

86) prolactin receptor

87) prostate specific antigen

88) prostatic acid phosphatase

89) protein kinase A

90) ribonucleotide diphosphate reductase

91) ribonucleotide reductase

92) seprase

93) sex hormone globulin binding receptor

94) sigma receptors

95) somatostatin receptors

96) SP220K

97) Src kinase

98) steroid sulfatase

99) stromelysin 3

100) sucrase-isomaltase

101) TADG14

102) Thiolesterase II

103) thrombin

104) thrombin receptor

105) thymidine kinase

106) thymidylate synthase

107) tissue factor

108) tissue plasminogen activator

109) TMPRSS2

110) transferrin receptors

111) transforming growth factors and their receptors

112) transporter (PEPT1)

113) trypsin

114) tumor necrosis factor receptor

115) type IV collagenase

116) uridine/cytidine kinase

117) urokinase

118) vacuolar type proton pump (V- ATPase)

119) xanthine-guanine phosphoribosyltransferase

120) any tumor-selective antigen

121) any tissue specific antigen which is present on tumor cells, but absent from vital normal cells

Tumor-selective Targets and Targeting Ligands:

The targeting ligands described below are preferred embodiments of targeting ligands for anti-cancer drugs ET of the present invention and all targeted anti-cancer drugs that are embodiments of the present invention:

Laminin Receptors

The laminin receptor is a membrane associated protein which binds laminin, elastin and, type IV collagen. The receptor facilitates the cell adhesion and migration key components of invasiveness characteristic of malignancy. The laminin receptor is over-expressed in a large number of malignancies including: breast, colon, prostate, ovarian, renal, pancreatic, melanoma, thyroid, lung, lymphomas, leukemias, gastric, and hepatocellular cancer. It is strongly associated with metastatic ability and is an independent adverse prognostic in breast, prostate, lung, thyroid and gastric cancer. The following references relate to this subject matter: Viacava P., et al., “The Spectrum of 67-kD Laminin Receptor Expression in Breast Carcinoma Progression,” J Pathol, 182:36-44 (1997); Menard S., et al., “Immunodetection of Bone Marrow Micrometastases in Breast Carcinoma Patients and its Correlation with Primary Tumour Prognostic Features,” Br J Cancer, 69(6):1126-9 (1994); Putz E., et al., “Phenotypic Characteristics of Cell Lines Derived from Disseminated Cancer Cells in Bone Marrow of Patients with Solid Epithelial Tumors: Establishment of Working Models for Human Micrometastases,” Cancer Res, 59(1):241-8 (1999); Hipfel R., et al., “Specifically Regulated Genes in Malignant Melanoma Tissues Identified by Subtractive Hybridization,” Br J Cancer, 82(6):1149-57 (2000); Pelosi G., et al., ‘High-Affinity Monomeric 67-Kd Laminin Receptors and Prognosis in Pancreatic Endocrine Tumours,” J Pathol, 183(1):62-9 (1997); Sanjuan X., et al., “Over-expression of the 67-kD Laminin Receptor Correlates with Tumour Progression in Human Colorectal Carcinoma,” J Pathol, 179(4):376-80 (1996); van den Brule F. A., et al., “Expression of the 67 kD Laminin Receptor in Human Ovarian Carcinomas as Defined by a Monoclonal Antibody, MLuC5,” Eur J Cancer, 32A(9):1598-602 (1996); Hand P. H., et al., “Expression of Laminin Receptor in Normal and Carcinomatous Human Tissues as Defined by a Monoclonal Antibody,” Cancer Res, 45(6):2713-9 (1985); Cioce V., et al., “Increased Expression of the Laminin Receptor in Human Colon Cancer,” J Natl Cancer Inst, 83:29-36 (1991); Massia S. P., et al., “Covalently Immobilized Laminin Peptide Tyr-Ile-Gly-Ser-Arg (YIGSR) Supports Cell Spreading and Co-Localization of the 67-Kilodalton Laminin Receptor with Alpha-Actinin and Vinculin,” J Biol Chem, 268(11):8053-9 (1993); Nadji M., et al., “Laminin Receptor in Lymph Node Negative Breast Carcinoma,” Cancer, 85(2):432-6 (1999); Terranova V. P., et al., “Laminin Receptor on Human Breast Carcinoma Cells,” Proc Natl Acad Sci USA, 80(2):444-8 (1983).Montuori N., et al., “Laminin Receptors in Differentiated Thyroid Tumors: Restricted Expression of the 67-Kilodalton Laminin Receptor in Follicular Carcinoma Cells,” J Clin Endocrinol Metab, 84(6):2086-92 (1999); Fontanini G., et al., “67-Kilodalton Laminin Receptor Expression Correlates with worse Prognostic Indicators in Non-Small Cell Lung Carcinomas,” Clin Cancer Res, 3(2):227-31 (1997); Menard S., et al., “New Insights into the Metastasis-Associated 67 kD Laminin Receptor,” J Cell Biochem, 6792):155-65 (1997); de Manzoni G., et al., “Prognostic Significance of 67-kDa Laminin Receptor Expression in Advanced Gastric Cancer,” Oncology, 55(5):456-60 (1998); Wewer U. M., et al., “Role of Laminin Receptor in Tumor Cell Migration,” Cancer Res, 47(21):5691-8 (1987); Menard S., et al., “The 67 kDa Laminin Receptor as a Prognostic Factor in Human Cancer,” Breast Cancer Res Treat, 52(1-3):137-45 (1998); Zheng S., et al., “The Relationship between 67KD Laminin Receptor Expression and Metastasis of Hepatocellular Carcinoma,” J Tongji Med Univ, 17(4):200-2 (1997); van den Brule F. A., et al., “Expression of the 67-kD Laminin Receptor, Galectin-1, and Galectin-3 in Advanced Human Uterine Adenocarcinoma,” Hum Pathol, 27(11):1185-91 (1996); Waltregny D., et al., “Brief Communication. Independent Prognostic Value of the 67-kD Laminin Receptor in Human Prostate Cancer,” J Natl Cancer Inst, 89(16):1224-1227 (1997), the contents of which are incorporated herein by reference in their entirety.

The laminin receptor, although highly over-expressed in many malignancies, is a normal cellular component of many tissues especially endothelial cells. The very low levels of laminin receptor in normal bone marrow cells is of significance as bone marrow toxicity is dose limiting for most anti-cancer drugs. The following references relate to this subject matter: Hand P. H., et al., “Expression of Laminin Receptor in Normal and Carcinomatous Human Tissues as Defined by a Monoclonal Antibody,” Cancer Res, 45(6):2713-9 (1985);Hilario E., et al., “Presence of Laminin and 67kDa Laminin-Receptor on Endothelial Surface of Lung Capillaries. An Immunocytochemical Study,” Histol Histopathol, 11 (4):915-8 (1996); Montuori N., et al., “Expression of the 67-kDa Laminin Receptor in Acute Myeloid Leukemia Cells Mediates Adhesion to Laminin and is Frequently Associated with Monocytic Differentiation,” Clin Cancer Res, 5(6): 1465-72 (1999), the contents of which are incorporated herein by reference in their entirety.

The laminin receptor binds with high affinity to a number of oligopeptides that are related to laminin or elastin. Laminin receptor antagonists have been shown to inhibit metastasis in animals. Radiolabelled laminin binding analogs and monoclonal antibodies specific for the laminin receptor have been explored as potential diagnostic and or therapeutic agents. The following references relate to this subject matter: Maeda M., et al., “Amino Acids and Peptides. XXXIII. A Bifunctional Poly(Ethylene Glycol) Hybrid of Laminin-Related Peptides,” Biochem Biophys Res Commun, 248(3):485-9 (1998); Graf J., et al., “A Pentapeptide from the Laminin B1 Chain Mediates Cell Adhesion and Binds the 67,000 Laminin Receptor,” Biochemistry, 26(22):6896-900 (1987); Rahman A., et al., “Anti-Laminin Receptor Antibody Targeting of Liposomes with Encapsulated Doxorubicin to Human Breast Cancer Cells in Vitro,” J Natl Cancer Inst, 81:1794-1800 (1989); Mu Y., et al., “Bioconjugation of Laminin Peptide YIGSR with Poly(Styrene Co-Maleic Acid) Increases its Antimetastatic Effect on Lung Metastasis of B16-BL6 Melanoma Cells,” Biochem Biophys Res Commun, 255(1):75-9 (1999); Mu Y., et al., “Bioconjugation of Laminin-Related Peptide YIGSR with Polyvinyl Pyrrolidone Increases its Antimetastatic Effect due to a Longer Plasma Half-Life,” Biochem Biophys Res Commun, 264(3):763-7 (1999); Iwamoto Y., et al., “YIGSR, a Synthetic Laminin Peptide, Inhibits the Enhancement by Cyclophosphamide of Experimental Lung Metastasis of Human Fibrosarcoma Cells,” Clin Exp Metastasis, 10(3):183-9 (1992); Massia S. P., et al., “Covalently Immobilized Laminin Peptide Tyr-lle-Gly-Ser-Arg (YIGSR) Supports Cell Spreading and Co-Localization of the 67-Kilodalton Laminin Receptor with Alpha-Actinin and Vinculin,” J Biol Chem, 268(11):8053-9 (1993); Koliakos G., et al., “Lung Carcinoma Imaging using a Synthetic Laminin Derivative Radioiodinated Peptide YIGSR,” J Nucl Med, 38(12):1940-4 (1997); Zhao M., et al., “Synthetic Laminin-Like Peptides and Pseudopeptides as Potential Antimetastatic Agents,” J Med Chem, 37(20):3383-8 (1994); Hinek A., et al., “The 67-kD Elastin/Laminin-Binding Protein is Related to an Enzymatically Inactive, Alternatively Spliced Form of Beta-Galactosidase,” J Clin Invest, 91(3):1198-205 (1993); Iwamoto Y., et al., “YIGSR, a Synthetic Laminin Pentapeptide, Inhibits Experimental Metastasis Formation,” Science, 238(4830):132-4 (1987); Blood C. H., et al., “Identification of a Tumor Cell Receptor for VGVAPG, an Elastin-Derived Chemotactic Peptide,” J Cell Biol, 107(5):1987-93 (1988); Grosso L. E.; Scott M., “PGAIPG, a Repeated Hexapeptide of Bovine and Human Tropoelastin, is Chemotactic for Neutrophils and Lewis Lung Carcinoma Cells,” Arch Biochem Biophys, 305(2):401-4 (1993); Grosso L. E.; Scott M., “Peptide Sequences Selected by BA4, a Tropoelastin-Specific Monoclonal Antibody, are Ligands for the 67-Kilodalton Bovine Elastin Receptor,” Biochemistry, 32(48):13369-74 (1993); Mecham R. P., et al., “Elastin Binds to a Multifunctional 67-Kilodalton Peripheral Membrane Protein,” Biochemistry, 28(9):3716-22 (1989); Mecham R. P., et al., “The Elastin Receptor Shows Structural and Functional Similarities to the 67-kDa Tumor Cell Laminin Receptor,” J Biol Chem, 264(28):16652-7 (1989); 5,567,408, 10/22196, Zamora, “YIGSR Peptide Radiopharmaceutical Applications”; U.S. Pat. No. 5,556,609, Sep. 17, 1996, Zamora, “YIGSR Peptide Radiopharmaceutical Applications”; U.S. Pat. No. 5,759,515, Jun. 2, 1998, Rhodes, et al., “Polyvalent Peptide Pharmaceutical Applications”; U.S. Pat. No. 5,231,082, Jul. 27, 1993, Schasteen, “Cyclic Peptide with Anti-Metastasis Activity”; U.S. Pat. No. 5,092,885, Mar. 3, 1992, Yamada, et al., “Peptides with Laminin Activity”; U.S. Pat. No. 5,039,662, Aug. 13, 1991, Schasteen, “Peptide with Anti-Metastasis Activity”; U.S. Pat. No. 4,565,789, Jan. 21, 1986, Liotta, et al., “Cell Matrix Receptor System and Use in Cancer Diagnosis and Management”, the contents of which are incorporated herein by reference in their entirety.

A preferred embodiment of the present invention is a compound ET with a targeting ligand comprised of a structure that binds to a laminin receptor binding ligand.

In preferred embodiments (embodiments TL1, TL2, TL3, TL4, and TL5), the targeting ligand comprises the following structures:

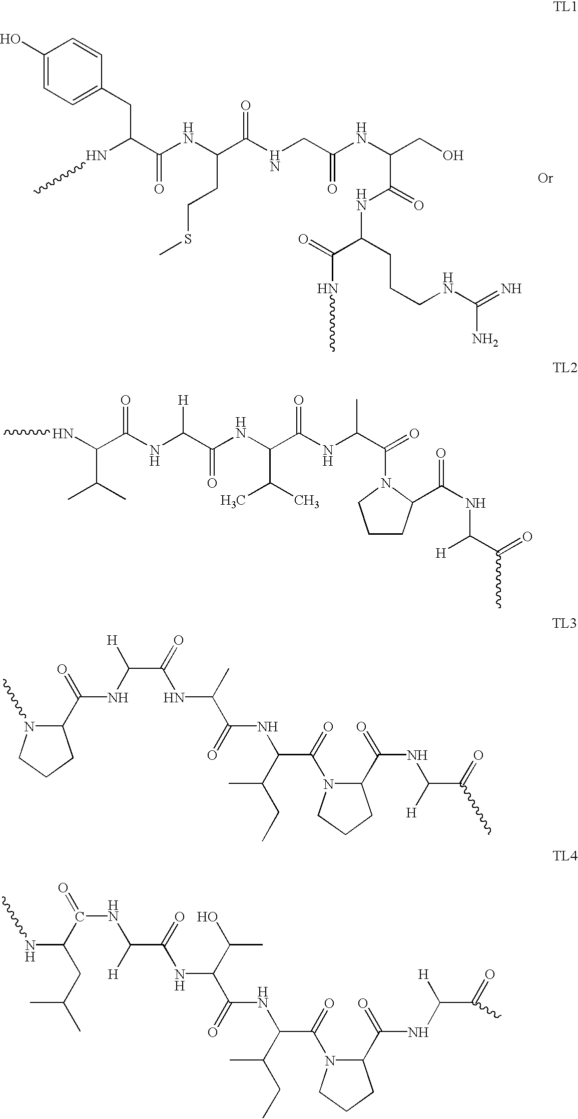

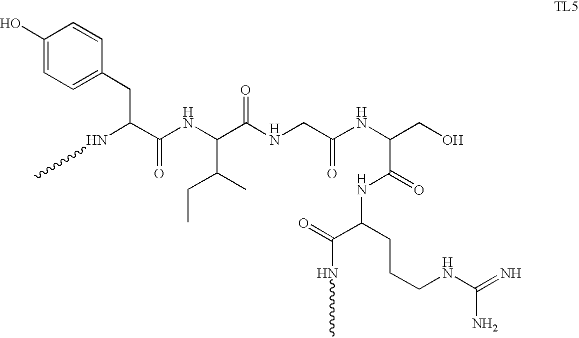

wherein the wavy line is H, OH, NH₂, or the site of linker attachment to the remainder of the drug complex; and wherein the amino acid residues have the L-configuration, or the D configuration, or are a racemic mixture.

Integrin alpha V beta 3

Integrin alpha V beta 3 (αᵥβ₃) are cell adhesion molecules which bind to RGD peptide sequences present in many extracellular matrix proteins. αᵥβ₃ is over-expressed on tumor cells in a number of important malignancies including: melanoma, breast cancer metastatic to bone, ovarian cancer, and neuroblastoma. In addition, αᵥβ₃ over-expressed by endothelial cells in tumor neovasculature. αᵥβ₃ expression is a strong adverse prognostic indicator in patients with breast cancer. αᵥβ₃ is not unique to tumors or tumor neovasculature and is also expressed by platlets, osteoclasts, endothelial cells during wound repair, and by vascular smooth muscle cells. Antagonists and monoclonal antibodies to αᵥβ₃ inhibit angiogenesis and tumor growth. Radiolabelled ligands for αᵥβ₃ have been described as potential tumor imaging agents. Doxorubicin conjugates of integrin ligands have been described as potential anti-cancer drugs. Monoclonal antibodies to αᵥβ₃ are used to reduce coronary artery restenosis following angioplasty. The following references relate to this subject matter: Horton M. A., et al., “The Alpha V Beta 3 Integrin ‘Vitronectin Receptor’,” Int J Biochem Cell Biol, 29(5):721-5 (1997); Pasqualini R, et al., “Alpha V Integrins as Receptors for Tumor Targeting by Circulating Ligands,” Nat Biotechnol, 15(6):542-6 (1997); Luna J., et al., “Antagonists of Integrin Alpha v Beta 3 Inhibit Retinal Neovascularization in a Murine Model,” Lab Invest, 75(4):563-73 (1996); Brooks P. C., et al., “Antiintegrin Alpha V Beta 3 Blocks Human Breast Cancer Growth and Angiogenesis in Human Skin,” J Clin Invest, 96(4):1815-22 (1995); Rabb H., et al., “Alpha-V/beta-3 and alpha-V/beta-5 Integrin Distribution in Neoplastic Kidney,” Am J Nephrol, 16(5):402-8 (1996).Timar J., et al., “Expression and Function of the High Affinity AlphaII/beta3 Integrin in Murine Melanoma Cells,” Clin Exp Metastasis, 16(5):437-45 (1998); Deryugina E. I., et al., “Functional Activation of Integrin Alpha V Beta 3 in Tumor Cells Expressing Membrane-Type 1 Matrix Metalloproteinase,” Int J Cancer, 86(1):15-23 (2000); Gladson C. L., et al., “Expression of Integrin Alpha v Beta 3 in Small Blood Vessels of Glioblastoma Tumors,” J Neuropathol Exp Neurol, 55(11):1143-9 (1996); Platten M., et al., “Transforming Growth Factors Beta(1) (TGF-beta(1)) and TGF-beta(2) Promote Glioma Cell Migration via Up-Regulation of Alpha(V)Beta(3) Integrin Expression,” Biochem Biophys Res Commun, 268(2):607-11 (2000); Trusolino L., et al., “Growth Factor-Dependent Activation of Alphavbeta3 Integrin in Normal Epithelial Cells: Implications for Tumor Invasion,” J Cell Biol, 142(4):1145-56 (1998); Max R., et al., “Immunohistochemical Analysis of Integrin Alpha Vbeta3 Expression on Tumor-Associated Vessels of Human Carcinomas,” Int J Cancer, 71(3):320-4 (1997); Racanelli A. L., et al., “Inhibition of Neointima Formation by a Nonpeptide Alpha(V)Beta(3) Integrin Receptor Antagonist in a Rabbit Cuff Model,” J Cell Biochem, 77(2):213-20 (2000); Liapis H., et al., “Integrin Alpha V Beta 3 Expression by Bone-Residing Breast Cancer Metastases,” Diagn Mol Pathol, 5(2):127-35 (1996); Brassard D. L., et al., Integrin Alpha(V)Beta(3)-Mediated Activation of Apoptosis,” Exp Cell Res, 251(1):33-45 (1999); Brooks P. C., et al., “Localization of Matrix Metalloproteinase MMP-2 to the Surface of Invasive Cells by Interaction with Integrin Alpha V Beta 3,” Cell, 85(5):683-93 (1996); DeNardo S. J., et al., “Neovascular Targeting with Cyclic RGD Peptide (Crgdf-ACHA) to Enhance Delivery of Radioimmunotherapy,” Cancer Biother Radiopharm, 15(1):71-9 (2000); Kerr J. S., et al., “Novel Small Molecule Alpha V Integrin Antagonists: Comparative Anti-Cancer Efficacy with Known Angiogenesis Inhibitors,” Anti-cancer, 19(2A):959-68 (1999); Liapis H., et al., “Expression of Alpha(V)Beta3 Integrin is Less Frequent in Ovarian Epithelial Tumors of Low Malignant Potential in Contrast to Ovarian Carcinomas,” Hum Pathol, 28(4):443-9 (1997); Gasparini G., et al., “Vascular Integrin Alpha(V)Beta3: A New Prognostic Indicator in Breast Cancer,” Clin Cancer Res, 4(11):2625-34 (1998); Lanza P., et al., “Selective Interaction of a Conformationally-Constrained Arg-Gly-Asp (RGD) Motif with the Integrin Receptor Alphavbeta3 Expressed on Human Tumor Cells,” Blood Cells Mol Dis, 23(2):230-41 (1997); Romanov V. I. et al., “RGD-Recognizing Integrins Mediate Interactions of Human Prostate Carcinoma Cells with Endothelial Cells in Vitro,” Prostate, 39(2):108-18 (1999); Trikha M., et al., “Role of Alphall(B)Beta3 Integrin in Prostate Cancer Metastasis,” Prostate, 35(3):185-92 (1998); Cheresh D. A., “Structure, Function and Biological Properties of Integrin Alpha V Beta 3 on Human Melanoma Cells,” Cancer Metastasis Rev, 10(1):3-10 (1991); Shahan T. A., et al., “Regulation of Tumor Cell Chemotaxis by Type IV Collagen is Mediated by a Ca(2+)-Dependent Mechanism Requiring CD47 and the Integrin Alpha(V)Beta(3),” J Biol Chem, 275(7):4796-802 (2000); Singh B., et al., “Vascular Expression of the Alpha(V)Beta(3)-lntegrin in Lung and Other Organs,” Am J Physiol Lung Cell Mol Physiol, 278(1):L217-26 (2000); Clark R. A., et al., “Transient Functional Expression of Alphavbeta 3 on Vascular Cells During Wound Repair,” Am J Pathol, 148(5):1407-21 (1996), the contents of which are incorporated herein by reference in their entirety.

A large number of compounds are known that bind with high affinity and selectivity to αᵥβ₃. The following references relate to this subject matter: Keenan R. M., et al., “Benzimidazole Derivatives as Arginine Mimetics in 1,4-Benzodiazepine Nonpeptide Vitronectin Receptor (Alpha V Beta 3) Antagonists,” Bioorg Med Chem Lett, 8(22):3165-70 (1998); Hart S. L., et al., “Cell Binding and Internalization by Filamentous Phage Displaying a Cyclic Arg-Gly-Asp-Containing Peptide,” J Biol Chem, 269(17):12468-74 (1994); Keenan R. M., et al., “Conformational Preferences in a Benzodiazepine Series of Potent Nonpeptide Fibrinogen Receptor Antagonists,” J Med Chem, 42(4):545-59 (1999); Nicolaou K. C., et al., “Design, Synthesis and Biological Evaluation of Nonpeptide Integrin Antagonists,” Bioorg Med Chem, 6(8):1185-208 (1998); Keenan R. M., et al., “Discovery of Potent Nonpeptide Vitronectin Receptor (Alpha V Beta 3) Antagonists,” J Med Chem, 40(15):2289-92 (1997); Bitan G., et al., “Design and Evaluation of Benzophenone-Containing Conformationally Constrained Ligands as Tools for Photoaffinity Scanning of the Integrin Alphavbeta3-Ligand Bimolecular Interaction,” J Pept Res, 55(3):181-94 (2000); Rockwell A. L., et al., “Rapid Synthesis of RGD Mimetics with lsoxazoline Scaffolds on Solid Phase: Identification of Alphavbeta3 Antagonists Lead Compounds,” Bioorg Med Chem Lett, 9(7):937-42 (1999); Keenan R. M., et al., “Orally Bioavailable Nonpeptide Vitronectin Receptor Antagonists Containing 2-Aminopyridine Arginine Mimetics,” Bioorg Med Chem Lett, 9(13):1801-6 (1999); Burgess K., et al., “Synthesis and Solution Conformation of Cyclo[RGDRGD]: a Cyclic Peptide with Selectivity for the Alpha V Beta 3 Receptor,” J Med Chem, 39(22):4520-6 (1996); Yamada T., et al., “Tailoring Echistatin to Possess Higher Affinity for Integrin alpha(IIb)beta(3),” FEBS Lett, 387(1):11-15 (1996); WO 96-US13500, 1997, Ruminski P. G., et al., “Preparation of Meta-Guanidine, Urea, Thiourea or Azacyclic Amino Benzoic Acid Derivatives as Integrin Antagonists”; Carron C. P., et al., “A Peptidomimetic Antagonist of the Integrin αᵥβ₃ Inhibits Leydig Cell Tumor Growth and the Development of Hypercalcemia of Malignancy,” Cancer Res, 58(9):1930-1935 (1998), the contents of which are incorporated herein by reference in their entirety.

A preferred embodiment of the present invention is a compound ET with a targeting ligand comprised of a structure that binds to αᵥβ₃. In preferred embodiments, (embodiments TL6, TL7, and TL8) the targeting ligand is comprised of one of the following structures:

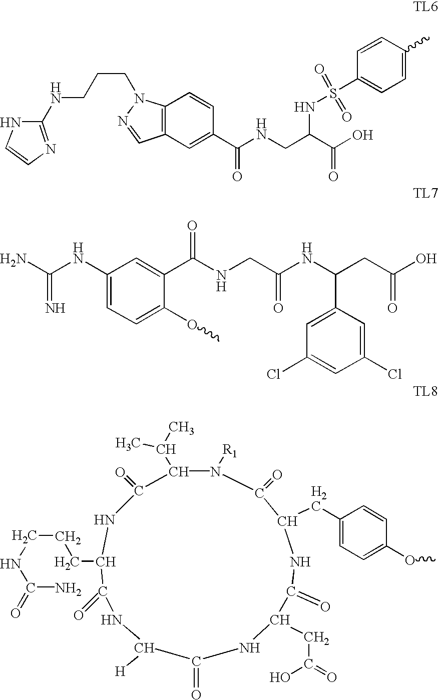

wherein the wavy line is the site of linker attachment to the remainder of the drug complex and R₁ is H, or methyl, and amino acids in the cyclopeptide are the L-configuration except for the tyrosine which is the D-configuration.

In preferred embodiments of the invention the targeting ligand for αᵥβ₃ is used in conjunction with targeting ligands that bind to other target receptors which are over-expressed on tumor neovasculature such as urokinase, plasmin, MMP-1-, MMP-3. MMP-9, membrane type −1 matrix metalloproteinase, or prostate specific membrane antigen.

Matrix Metalloproteinases as Targets

Matrix metalloproteases (MMP) are enzymes, which degrade connective tissue and which are over-expressed by a large number of tumors and stroma of tumors. There have been an enormous number of inhibitors to matrix metalloproteases developed as potential anti-cancer drugs. However, inhibition of MMP activity does not typically produce cytotoxicity and several clinical trials to date have failed to show efficacy of MMP inhibitors as antimetastatic drugs. At the present time, there are no known methods to convert the over-expression of MMPs into selective tumor toxicity. The following references relate to this subject matter: Nelson A. R., et al., “Matrix Metalloproteinases: Biologic Activity and Clinical Implications,” J Clin Oncol, 18(5):1135 (2000); Whittaker M., et al., “Design and Therapeutic Application of Matrix Metalloproteinase Inhibitors,” Chem Rev, 99:2735-2776 (1999); Curran S.; Murray G. I., “Matrix Metalloproteinases in Tumour Invasion and Metastasis,” J Pathol, 189(3):300-308 (1999), the contents of which are incorporated herein by reference in their entirety.

Membrane type metalloproteinases are associated with the cell surface by a hydrophobic transmembrane domains or glycosylphosphatidylinositol anchors. Other MMP's become associated with the surface of tumor cells by a variety of mechanisms which include binding to:

1.) MT-1-MMP and TIMP2 (tissue inhibitor of metalloproteinase);

2.) Heparin sulfate proteoglycans;

3.) Hyaluronan receptor CD44;

4.) Integrin alpha V beta 3; and

5.) Extracellular matrix metalloproteinase inducer (EMMPRIN) specific receptors.

Accordingly, ligands, which bind to MMP's, can be employed in targeting tumors. The following references relate to this subject matter: Sato H., et al., “Cell Surface Binding and Activation of Gelatinase a Induced by Expression of Membrane-Type-1-Matrix Metalloproteinase (MTI -MMP),” FEBS Lett, 385(3):238-40 (1996); Monsky W. L., et al., “Binding and Localization of M(r) 72,000 Matrix Metalloproteinase at Cell Surface Invadopodia,” Cancer Res, 53(13):3159-64 (1993); Yu Q; Stamenkovic I., “Cell Surface-Localized Matrix Metalloproteinase-9 Proteolytically Activates TGF-Beta and Promotes Tumor Invasion and Angiogenesis,” Genes Dev, 14(2):163-76 (2000); Menashi S., et al., “Density-dependent Regulation of Cell-Surface Association of Matrix Metalloproteinase-2 (MMP-2) in Breast-Carcinoma Cells,” Int J Cancer, 75(2):259-65 (1998); Deryugina E. I., et al., “Functional Activation of Integrin Alpha V Beta 3 in Tumor Cells Expressing Membrane-Type 1 Matrix Metalloproteinase,” Int J Cancer, 86(1):15-23 (2000); Guo H., et al., “EMMPRIN (CD147), an Inducer of Matrix Metalloproteinase Synthesis, also Binds Interstitial Collagenase to the Tumor Cell Surface,” Cancer Res, 60(4):888-91 (2000); Sawicki G., et al., “Expression of the Active Form of MMP-2 on the Surface of Leukemic Cells Accounts for their in Vitro Invasion,” J Cancer Res Clin Oncol, 124(5):245-52 (1998); Yu W. H.; Woessner J. F. Jr., “Heparan Sulfate Proteoglycans as Extracellular Docking Molecules for Matrilysin (Matrix Metalloproteinase 7),” J Biol Chem, 275(6):4183-91 (2000); Chen W. T.; Wang J. Y., “Specialized Surface Protrusions of Invasive Cells, Invadopodia and Lamellipodia, have Differential MT1-MMP, MMP-2, and TIMP-2 Localization,” Ann NY Acad Sci, 878:361-71 (1999); Brooks P. S., et al., “Localization of Matrix Metalloproteinase MMP-2 to the Surface of Invasive Cells by Interaction with Integrin Alpha V Beta 3,” Cell, 85(5):683-93 (1996); Yu Q; Stamenkovic I., “Localization of Matrix Metalloproteinase 9 to the Cell Surface Provides a Mechanism for CD44-Mediated Tumor Invasion,” Genes Dev, 13(1):35-48 (1999); Bourguignon L. Y., et al., “CD44v(3,8-10) is Involved in Cytoskeleton-Mediated Tumor Cell Migration and Matrix Metalloproteinase (MMP-9) Association in Metastatic Breast Cancer Cells,” J Cell Physiol, 176(1):206-15 (1998); Corcoran M. L., et al., “TIMP-2 Mediates Cell Surface Binding of MMP-2,” Adv Exp Med Biol,” 389:295-304 (1996); Emonard H. P., et al., “Tumor Cell Surface-Associated Binding Site for the M(R) 72,000 Type IV Collagenase,” Cancer Res, 52(20):5845-8 (1992); Barmina O. Y., et al., “Collagenase-3 Binds to a Specific Receptor and Requires the Low Density Lipoprotein Receptor-Related Protein for Internalization,” J Biol Chem, 274(42):30087-93 (1999), the contents of which are incorporated herein by reference in their entirety.

A preferred embodiment of the present invention is a compound ET with a targeting ligand comprised of a structure that binds to a matrix metalloproteinase.

Matrix Metalloproteinase 7 Selective Ligands:

Matrix Metalloproteinase 7 (MMP-7 or Matrilysin) is a protease, which is constitutively produced by exocrine epithelial cells. MMP-7 is over-expressed by tumor cells in wide range of malignancies including: ovarian, gastric, prostate, colorectal, endometrial, gliomas, and breast cancer. MMP-7 contrasts with many other metalloproteases, which are over-expressed by tumor stromal elements rather than the tumor cells. At the present time there are no known methods to convert the over-expression of MMP-7 into selective tumor toxicity. The following references relate to this subject matter: Yamamoto H., et al., “Association of Matrilysin Expression with Recurrence and Poor Prognosis in Human Esophageal Squamous Cell Carcinoma,” Cancer Res, 59(14):3313-6 (1999); Adachi Y., et al., “Contribution of Matrilysin (MMP-7) to the Metastatic Pathway of Human Colorectal Cancers,” Gut, 45(2):252-8 (1999); Yamashita K, et al., “Expression and Tissue Localization of Matrix Metalloproteinase 7 (Matrilysin) in Human Gastric Carcinomas. Implications for Vessel Invasion and Metastasis,” Int J Cancer, 79(2):187-94 (1998); Pacheco M. M., et al., “Expression of Gelatinases A and B, Stromelysin-3 and Matrilysin Genes in Breast Carcinomas: Clinico-Pathological Correlations,” Clin Exp Metastasis, 16(7):577-85 (1998); Hashimoto K., et al., “Expression of Matrix Metalloproteinase-7 and Tissue Inhibitor of Metalloproteinase-1 in Human Prostate,” J Urol, 160(5):1872-6 (1998); Mori M., et al., “Over-expression of Matrix Metalloproteinase-7 mRNA in Human Colon Carcinomas,” Cancer, 75(6 Suppl):1516-9 (1995); Honda M., et al., “Matrix Metalloproteinase-7 Expression in Gastric Carcinoma,” Gut, 39(3):444-8 (1996); Nakano A., et al., “[Increased Expression of Gelatinases A and B, Matrilysin and TIMP-1 Genes in Human Malignant Gliomas],” Nippon Rinsho, 53(7):1816-21 (1995); Knox J. D., et al., “Matrilysin Expression in Human Prostate Carcinoma,” Mol Carcinog, 15(1):57-63 (1996); Adachi Y., et al., “Matrix Metalloproteinase Matrilysin (MMP-7) Participates in the Progression of Human Gastric and Esophageal Cancers,” Int J Oncol, 13(5):1031-5 (1998); Ueno H., et al., “Enhanced Production and Activation of Matrix Metalloproteinase-7 (Matrilysin) in Human Endometrial Carcinomas,” Int J Cancer, 84(5):470-7 (1999); Barille S., et al., “Production of Metalloproteinase-7 (Matrilysin) by Human Myeloma Cells and its Potential Involvement in Metalloproteinase-2 Activation,” J Immunol, 163(10):5723-8 (1999); Senota A., et al.,” Relation of Matrilysin Messenger RNA Expression with Invasive Activity in Human Gastric Cancer,” Clin Exp Metastasis, 16(4):313-21 (1998); Saarialho-Kere U. K., et al., “Matrix Metalloproteinase Matrilysin is Constitutively Expressed in Adult Human Exocrine Epithelium,” J Invest Dermatol, 105(2):190-6 (1995); Tanimoto H., et al., “The Matrix Metalloprotease Pump-1 (MMP-7, Matrilysin): A Candidate Marker/Target for Ovarian Cancer Detection and Treatment,” Tumour Biol, 20(2):88-98 (1999), the contents of which are incorporated herein by reference in their entirety.

In a preferred embodiment, An is a ligand for MMP-7. A large number of potent reversible ligands are known that reversibly inhibit MMP-7. The following references relate to this subject matter: Whittaker M., et al., “Design and Therapeutic Application of Matrix Metalloproteinase Inhibitors,” Chem Rev, 99:2735-2776 (1999); Pratt L. M., et al., “The Synthesis of Novel Matrix Metalloproteinase Inhibitors Employing the Ireland-Claisen Rearrangement,” Bioorg Med Chem Lett, 8:1359-1364 (1998); Abramson S. R., et al., “Characterization of Rat Uterine Matrilysin and Its cDNA,” J Biological Chem, 270(27):16016-16022 (1995); Nelson A. R., et al., “Matrix Metalloproteinases: Biologic Activity and Clinical Implications,” J Clin Oncology, 18(5):1135-1149 (2000), the contents of which are incorporated herein by reference in their entirety.

Preferred embodiment (embodiment TL9 and TL1 0) of the present invention is a compound ET with a targeting ligand comprised of a structure that binds to MMP-7 comprised of the following structure:

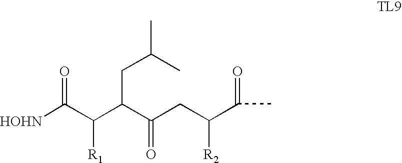

wherein the dotted line is the site of attachment or linker attachment to the remainder of the drug complex and wherein R₁ is hydroxy, methyl, ethyl, isopropyl, cyclopentyl, 3-(tetrahydrothiophenyl), or thiopen-2-ylthiomethyl-, and wherein R2 is benzyl, t-butyl, or isopropyl. These ligands can also bind to a number of other MMP's that are enriched in tumors.

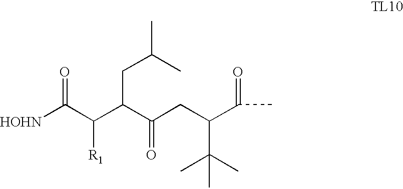

MMP1, 2, 3, 9 and Membrane Type 1 MMP. Targeting Ligands:

MMP 1, 2, 3, 9 and membrane type MMP 1(MT-MMP-1) are all over-expressed in a wide variety of malignancies. The following references relate to this subject matter: Stearns M.; Stearns M. E., “Evidence for Increased Activated Metalloproteinase 2 (MMP-2a) Expression Associated with Human Prostate Cancer Progression,” Oncol Res, 8(2):69-75 (1996); Moll U. M., et al., “Localization of Collagenase at the Basal Plasma Membrane of a Human Pancreatic Carcinoma Cell Line,” Cancer Res, 50(21):6995-70 (1990); Poulsom R., et al., “Expression of Gelatinase A and TIMP-2 mRNAs in Desmoplastic Fibroblasts in Both Mammary Carcinomas and Basal Cell Carcinomas of the Skin,” J Clin Pathol, 46(5):429-36 (1993); Jones, J. L., et al., “Expression of MMP-2 and MMP-9, Their Inhibitors, and the Activator MT1-MMP in Primary Breast Carcinomas,” J Pathol, 189(2):161-168 (1999); Polette M., et al., “Gelatinase A Expression and Localization in Human Breast Cancers. An in Situ Hybridization Study and Immunohistochemical Detection using Confocal Microscopy,” Virchows Arch, 424(6):641-5 (1994); Ohtani H., et al., “Immunoelectron Microscopic Localization of Gelatinase A in Human Gastrointestinal and Skin Carcinomas: Difference Between Cancer Cells and Fibroblasts,” Jpn J Cancer Res, 86(3):304-9 (1995); Montironi R., et al., “Immunohistochemical Evaluation of Type IV Collagenase (72-Kd Metalloproteinase) in Prostatic Intraepithelial Neoplasia,” Anti-cancer Res, 16(4A):2057-62 (1996); Stearns M. E.; Stearns M., “Immunohistochemical Studies of Activated Matrix Metalloproteinase-2 (MMP-2a)Expression in Human Prostate Cancer,” Oncol Res, 8(2):63-7 (1996); Caudroy S., et al., “Expression of the Extracellular Matrix Metalloproteinase Inducer (EMMPRIN) and the Matrix Metalloproteinase-2 in Bronchopulmonary and Breast Lesions,” J Histochem Cytochem, 47:1575-1580 (1999); Montironi R., et al., “Location of 72-kd Metalloproteinase (Type IV Collagenase) in Untreated Prostatic Adenocarcinoma,” Pathol Res Pract, 191(11):1140-6 (1995); Hamdy F. C., et al., “Matrix Metalloproteinase 9 Expression in Primary Human Prostatic Adenocarcinoma and Benign Prostatic Hyperplasia,” Br J Cancer, 69(1):177-82 (1994); Nelson A. R., et al., “Matrix Metalloproteinases: Biologic Activity and Clinical Implications,” J Clin Oncol, 18(5):1135 (2000); Emonard H. P. et al., “Tumor Cell Surface-Associated Binding Site for the M(R) 72,000 Type IV Collagenase,” Cancer Res, 52(20):5845-8 (1992); Bramhall S. R., et al., “Imbalance of Expression of Matrix Metalloproteinases (MMPs) and Tissue Inhibitors of the Matrix Metalloproteinases (TIMPs) in Human Pancreatic Carcinoma,” J Pathol, 182(347-355 (1997); Väiänen A., et al., “Prognostic Value of MMP-2 Immunoreactive Protein (72kD Type IV Collagenase) in Primary Skin Melanoma,” J Pathol, 186:51-58 (1998); Murray G. I., et al., “Matrix Metalloproteinases and their Inhibitors in Gastric Cancer,” Gut, 43(6):791-7 (1998); Lebeau A., et al., “Tissue Distribution of Major Matrix Metalloproteinases and their Transcripts in Human Breast Carcinomas,” Anti-cancer Res, 19(5B):4257-64 (1999); Murray G. I., et al., “Matrix Metalloproteinase-1 is Associated with Poor Prognosis in Oesophageal Cancer,” J Pathol, 185:256-261 (1998); Guo H., et al., “Emmprin (CD147), an Inducer of Matrix Metalloproteinase Synthesis, also Binds Interstitial Collagenase to the Tumor Cell Surface,” Cancer Res, 60(4):888-91 (2000), the contents of which are incorporated herein by reference in their entirety.

Similarities in the active site of these enzymes allow for targeting with a common family of ligands. Compounds of the following structure bind reversibly to MMP 1, 2, 3, 9 and membrane type MMP 1 with IC₅₀ in the nanomolar to subnanomolar range.

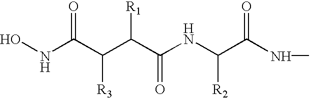

wherein R₁ is —CH₂CH(CH₃)₂, —(CH₂)₂C₆H₅, —(CH₂)₃C₆H₅, n-butyl, n-hexyl, n-octyl, R₂ is C₆H₅, - - - C₆H₁₁, —C(CH₃)₃, (indol-3-yl)methyl, —CH₂C₆H₅, (5, 6, 7, 8 -terahydro-1-napthyl)methyl, —CH(CH₃)₂, 1-(napthyl)methyl, 3-(napthyl)methyl, 1-(quinolyl)methyl, 3-(quinolyl)methyl, 3-pyridylmethyl, 4-pyridylmethyl, t-butyl, and R₃ is H, OH, methyl, 2-thienylthiomethyl, or allyl. The following references relate to this subject matter: Yamamoto M., et al., “Inhibition of Membrane-Type 1 Matrix Metalloproteinase by Hydroxamate Inhibitors: An Examination of the Subsite Pocket,” J Med Chem, 41:1209-1217 (1998); Curtin M. L., et al., “Broad Spectrum Matrix Metalloproteinase Inhibitors: An Examination of Succinamide Hydroxamate Inhibitors with P₁C_{α} Gem-Disubstitution,” Biorg Med Chem Lett, 8:1443-1448 (1998); Levy D. E., et al., “Matrix Metalloproteinase Inhibitors: A Structure-Activity Study,” J Med Chem, 41:199-223 (1998), the contents of which are incorporated herein by reference in their entirety.

A preferred embodiment of the present invention is a compound ET with a targeting ligand comprised of a structure that binds to MMP1, 2, 3, 9 or MT-MMP-1. In preferred embodiments, the targeting ligand comprises the following structure:

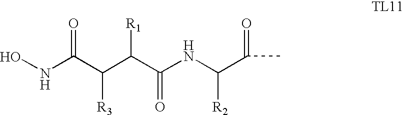

wherein the dotted line is the site of linker attachment to the remainder of the drug complex wherein R₁ is —CH₂CH(CH₃)₂, —(CH₂)₂C₆H₅, —(CH₂)₃C₆H₅, n-butyl, n-hexyl, n-octyl, R₂ is C₆H₅, - - - C₆H₁₁, —C(CH₃)₃, (indol-3-yl)methyl, —CH₂C₆H₅, (5, 6, 7, 8-terahydro-1-napthyl)methyl, —CH(CH₃)₂, 1-(napthyl)methyl, 3-(napthyl)methyl, 1-(quinolyl)methyl, 3-(quinolyl)methyl, 3-pyridylmethyl, 4-pyridylmethyl, t-butyl, and R₃ is H, OH, methyl, 2-thienylthiomethyl, or allyl.

In preferred embodiments (embodiment TL12), the targeting ligand comprises the following structures:

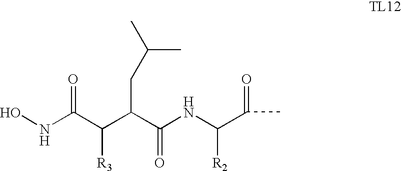

wherein R₂ is benzyl and R₃ is 2-thienylthiomethyl; or wherein R₂ is 5, 6, 7, 8,-terahydro-1-napthyl)methyl and R₃ is methyl; or wherein R₂ is t-butyl and R₃ is OH; or wherein R₂ is H and R₃ is (indol-3-yl)methyl; and wherein the dotted line is the site of linker attachment to the remainder of the drug complex.

Another preferred embodiment is based on diphenlyether sulfone inhibitors of MMP's, which are highly active against MMP2, 3, 9, 12, and 13 MMP. The following references relate to this subject matter: U.S. Pat. No. 5,932,595, Aug. 3, 1999, Bender et al., “Matrix Metalloprotease Inhibitors”; Lovejoy B., et al., “Crystal Structures of MMP-1 and -13 Reveal the Structural Basis for Selectivity of Collagenase Inhibitors,” Nat Struct Biol, 6(3):217-21 (1999); botos I., et al., “Structure of Recombinant Mouse Collagenase-3 (MMP-13),” J Mol Biol, 292:837-844 (1999), the contents of which are incorporated herein by reference in their entirety.

MMP 13 is an attractive target as it is over-expressed in a wide range of malignancies. The following references relate to this subject matter: Pendas A. M., et al., “An overview of Collagenase-3 Expression in Malignant Tumors and Analysis of its Potential Value as a Target in Antitumor Therapies,” Clin Chim Acta, 291(2):137-55 (2000); Shalinsky D. R., et al., “Broad Antitumor and Antiangiogenic Activities of AG3340, a Potent and Selective MMP Inhibitor Undergoing Advanced Oncology Clinical Trials,” Ann NY Acad Sci, 878:236-70 (1999); Johansson N., et al., “Collagenase-3 (MMP-13) is Expressed by Tumor Cells in Invasive Vulvar Squamous Cell Carcinomas,” Am J Pathol, 154(2):469-80 (1999); Barmina O. Y., et al., “Collagenase-3 Binds to a Specific Receptor and Requires the Low Density Lipoprotein Receptor-Related Protein for Internalization,” J Biol Chem, 274(42):30087-93 (1999); Cazorla M., et al., “Collagenase-3 Expression is Associated with Advanced Local Invasion in Human Squamous Cell Carcinomas of the Larynx,” J Pathol, 186(2):144-150 (1998); Balbin M., et al., “Expression and Regulation of Collagenase-3 (MMP-13) in Human Malignant Tumore,” APMIS, 107(1):45-53 (1999); Johansson N., et al., “Expression of Collagenase-3 (Matrix Metalloproteinase-13) in Squamous Cell Carcinomas of the Head and Neck,” Am J Pathol, 151(2):499-508 (1997); Uria J. A., et al., “Regulation of Collagenase-3 Expression in Human Breast Carcinomas is Mediated by Stromal-Epithelial Cell Interactions,” Cancer Res, 57(21):4882-8 (1997); Airola K., et al., “Human Collagenase-3 is Expressed in Malignant Squamous Epithelium of the Skin,” J Invest Dermatol, 109:225-231 (1997); Freije J. M., et al., “Molecular Cloning and Expression of Collagenase-3, A Novel Human Matrix Metalloproteinase Produced by Breast Carcinomas,” J Biol Chem, 269:24):16766-73 (1994); Uria J. A., et al., “Regulation of Collagenase-3 Expression in Human Breast Carcinomas is Mediated by Stromal-Epithelial Cell Interactions,” Cancer Res, 57(2):4882-8 (1997), the contents of which are incorporated herein by reference in their entirety.

A preferred embodiment of the present invention is a compound ET with a targeting ligand comprised of a structure that binds to MMP13. In preferred embodiments (TL13, TL14, and TL15), the targeting ligand comprises the following structure:

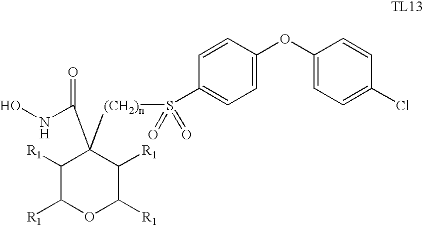

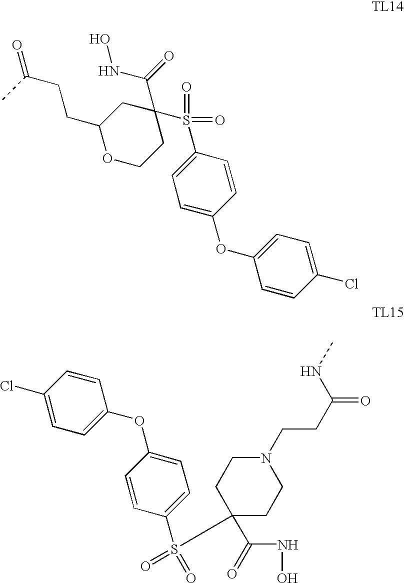

wherein n=0 or 1 and wherein R₁ is H, or the site of linker attachment to the remainder of the drug complex, and the dotted line is the site of linker attachment to the remainder of ET.

Urokinase Selective Ligands:

Urokinase is a serine protease, which converts plasminogen into enzymatically active plasmin. The enzyme binds to specific cell surface receptors and is over-expressed in most major types of cancers. Hepatocyte growth factor/Scatter Factor activation of the c-Met receptor, which is a characteristic of most malignancies, stimulates urokinase production. The overexpression of urokinase is a major adverse prognostic factor in multiple types of cancer including: breast, ovarian, prostate, colorectal, pancreatic, esophageal, gastric, renal, endometrial, and lung cancer. The expression of urokinase facilitates tissue invasion and metastasis. Depending upon the tumor type, urokinase can be located on tumor cells, stromal cells in the tumor, and on tumor-associated neovasculature. Urokinase, although an excellent marker of the malignant phenotype, is not unique to malignancy. Urokinase is constitutively expressed in the eye, kidney, testes, and in atherosclerotic vessels. The following references relate to this subject matter: Duffy M. J., et al., “Urokinase Plasminogen Activator: A Prognostic Marker in Multiple Types of Cancer,” J Surg Oncol, 71(2):130-5 (1999); Ploug M., et al., “Ligand Interaction between Urokinase-Type Plasminogen Activator and its Receptor Probed with 8-Anilino-1-Naphthalenesulfonate. Evidence for a Hydrophobic Binding Site Exposed only on the Intact Receptor,” Biochemistry, 33(30):8991-7 (1994); Shiomi H., et al., “Cellular Distribution and Clinical Value of Urokinase-Type Plasminogen Activator, its Receptor, and Plasminogen Activator Inhibitor-2 in Esophageal Squamous Cell Carcinoma,” Am J Pathol, 156(2):567-75 (2000); Harvey S. R., et al., “Demonstration of Urokinase Expression in Cancer Cells of Colon Adenocarcinomas by Immunohistochemistry and in Situ Hybridization,” Am J Pathol, 155(4):1115-20 (1999); Bouchet C., et al., “Dissemination Risk Index Based on Plasminogen Activator System Components in Primary Breast Cancer,” J Clin Oncol, 17(10):3048-57 (1999); Miyake H., et al., “Elevation of Urokinase-Type Plasminogen Activator and its Receptor Densities as New Predictors of Disease Progression and Prognosis in Men with Prostate Cancer,” Int J Oncol, 14(3):535-41 (1999); Dubuisson L., et al., “Expression and Cellular Localization of the Urokinase-Type Plasminogen Activator and its Receptor in Human Hepatocellular Carcinoma,” J Pathol, 190(2):190-5 (2000); Monvoisin A., et al., “Direct Evidence that Hepatocyte Growth Factor-Induced Invasion of Hepatocellular Carcinoma Cells is Mediated by Urokinase,” J Hepatol, 30(3):511-8 (1999); Kobayashi H, et al., “Increased Cell-Surface Urokinase in Advanced Ovarian Cancer,” Jpn J Cancer Res, 84(6):633-40 (1993); Yamamoto M., et al., “Increased Expression of Low Density Lipoprotein Receptor-Related Protein/Alpha2-Macroglobulin Receptor in Human Malignant Astrocytomas,” Cancer Res, 57(13):2799-805 (1997); Casslen B., et al., “Degradation of Urokinase Plasminogen Activator (UPA) in Endometrial Stromal Cells Requires both the UPA Receptor and the Low-Density Lipoprotein Receptor-Related Protein/Alpha2-Macroglobulin Receptor,” Mol Hum Reprod, 4(6):585-93 (1998); Nykjaer A., et al., “Mannose 6-phosphate/insulin-like Growth Factor-II Receptor Targets the Urokinase Receptor to Lysosomes via a Novel Binding Interaction,” J Cell Biol, 141(3):815-28 (1998); Noorman F., et al., “Degradation of Tissue-Type Plasminogen Activator by Human Monocyte-Derived Macrophages is Mediated by the Mannose Receptor and by the Low-Density Lipoprotein Receptor-Related Protein,” Blood, 86(9):3421-7 (1995); Wohn K. D., et al., “The Urokinase-Receptor (CD87) is Expressed in Cells of the Megakaryoblastic Lineage,” Thromb Haemost, 77(3):540-7 (1997); Volm M., et al., “Relationship of Urokinase and Urokinase Receptor in Non-Small Cell Lung Cancer to Proliferation, Angiogenesis, Metastasis and Patient Survival,” Oncol Rep, 6(3):611-5 (1999); Foekens J. A., et al., “The Urokinase System of Plasminogen Activation and Prognosis in 2780 Breast Cancer Patients,” Cancer Res, 60(3):636-43 (2000); Eatock, M. M., et al., “Tumour Vasculature as a Target for Anti-cancer Therapy,” Cancer Treat Rev, 26(3):191-204 (2000); Conese M., et al., “alpha-2 Macroglobulin Receptor/Ldl Receptor-Related Protein(Lrp)-Dependent Internalization of the Urokinase Receptor,” J Cell Biol, 131(6 Pt 1):1609-22 (1995); Duffy M J, et al. “Urokinase Plasminogen Activator as a Predictor of Aggressive Disease in Breast Cancer,” Enzyme Protein, 49(1-3):85-93 (1996); Fujii T., et al., “Urokinase-type Plasminogen Activator and Plasminogen Activator Inhibitor-1 as a Prognostic Factor in Human Colorectal Carcinomas,” Hepatogastroenterology, 46(28):2299-308 (1999); Brown P. A., et al., “Urokinase-Plasminogen Activator is Synthesized in Vitro by Human Glomerular Epithelial Cells but not by Mesangial Cells,” Kidney Int, 45(1):43-7 (1994); Gunnarsson M., et al., “Factors of the Plasminogen Activator System in Human Testis, as Demonstrated by In-Situ Hybridization and lmmunohistochemistry,” Mol Hum Reprod, 5(10):934-40 (1999); Falkenberg M., et al., “Localization of Fibrinolytic Activators and Inhibitors in Normal and Atherosclerotic Vessels,” Thromb Haemost, 75(6):933-8 (1996); Tripathi R. C., et al., “Localization of Urokinase-Type Plasminogen Activator in Human Eyes: An Immunocytochemical Study,” Exp Eye Res, 51(5):545-52 (1990); Wagner S. N., et al., “Sites of Urokinase-Type Plasminogen Activator Expression and Distribution of its Receptor in the Normal Human Kidney,” Histochem Cell Biol, 105(1):53-60 (1996), the contents of which are incorporated herein by reference in their entirety.

Since urokinase is such an important biochemical manifestation of the malignant phenotype, there have been extensive efforts to develop urokinase inhibitors and urokinase-targeted anti-cancer drugs. The following references relate to this subject matter: Jankun J., “Antitumor Activity of the Type I Plasminogen Activator Inhibitor and Cytotoxic Conjugate In Vitro,” Cancer Res, 52(20):5829-32 (1992); Ke S. H., et al., “Optimal Subsite Occupancy and Design of a Selective Inhibitor of Urokinase,” J Biol Chem, 272(33):20456-62 (1997); Ray P., et al., “Inhibitory Effect of Amiloride on the Urokinase Plasminogen Activators in Prostatic Cancer,” Tumour Biol, 19(1):60-4 (1998); Yang S. Q., et al., “Engineering Bidentate Macromolecular Inhibitors for Trypsin and Urokinase-Type Plasminogen Activator,” J Mol Biol, 279(4):1001-11 (1998); Christova E., et al., “Hydrophobic Interactions in the Urokinase Active Centre. Inhibitory Action of Alkyl Ammonium and Amidinium Ions: Comparison with Trypsin,” Int J Pept Protein Res, 15(5):459-63 (1980); Burgle M., et al., “Inhibition of the Interaction of Urokinase-Type Plasminogen Activator (uPA) with its Receptor (UPAR) by Synthetic Peptides,” Biol Chem, 378(3-4):231-7 (1997); Towle M. J., et al., “Inhibition of Urokinase by 4-Substituted Benzo[B]Thiophene-2-Carboxamidines: An Important New Class of Selective Synthetic Urokinase Inhibitor,” Cancer Res, 53(11):2553-9 (1993); Rabbani S. A., et al., “Prevention of Prostate-Cancer Metastasis In Vivo by a Novel Synthetic Inhibitor of Urokinase-Type Plasminogen Activator (uPA),” Int J Cancer, 63(6):840-5 (1995); Katz B. A., et al., “Structural Basis for Selectivity of a Small Molecule, S1-Binding, Submicromolar Inhibitor of Urokinase-Type Plasminogen Activator,” Chem Biol, 7(4):299-312 (2000); Bridges A. J., et al., “The Synthesis of Three 4-Substituted BenzoThiophene-2-Carboxamidines as Potent and Selective Inhibitors of Urokinase,” Bioorg Med Chem, 1(6):403-10 (1993); Billstrom A., et al., “The Urokinase Inhibitor P-Aminobenzamidine Inhibits Growth of a Human Prostate Tumor in SCID Mice,” Int J Cancer, 61(4):542-7 (1995); Evans D. M., et al., “Time and Dose Dependency of the Suppression of Pulmonary Metastases of Rat Mammary Cancer by Amiloride,” Clin Exp Metastasis, 16(4):353-7 (1998); Min H. Y., et al., “Urokinase Receptor Antagonists Inhibit Angiogenesis and Primary Tumor Growth in Syngeneic Mice,” Cancer Res, 56(10):2428-33 (1996); Fibbi G., et al., “Urokinase-Dependent Angiogenesis In Vitro and Diacylglycerol Production are Blocked by Antisense Oligonucleotides against the Urokinase Receptor,” Lab Invest, 78(9): 1109-19 (1998); Benzo[b]thiophene-2-carboxamidines: An Important New Class of Selective Synthetic Urokinase Inhibitor,” Cancer Res, 53(11):2553-9 (1993); U.S. Pat. No. 5,656,726, Aug. 12, 1997, Rosenberg, et al., “Peptide Inhibitors of Urokinase Receptor Activity”; Rabbani S A, et al., “Prevention of Prostate-Cancer Metastasis In Vivo by a Novel Synthetic Inhibitor of Urokinase-Type Plasminogen Activator (uPA),” Int J Cancer, 63(6):840-5 (1995); Billstrom A., et al., “The Urokinase Inhibitor p-Aminobenzamidine Inhibits Growth of a Human Prostate Tumor in SCID Mice,” Int J Cancer, 61(4):542-7 (1995); U.S. Pat. No. 5,747,458, May 5, 1998, Rosenberg, et al., “Urokinase Receptor Ligands”; Schmitt M., “Urokinase-Type Plasminogen Activator (uPA) and its Receptor (CD87): A New Target in Tumor Invasion and Metastasis,” J Obstet Gynaecol, 21(2):151-65 (1995); 5,679,350, 10/21/97, Jankun, et al., “Method of Delivery of a Medicament to a cancer Cell using a Pathway of Plasminogen Activator Material”; U.S. Pat. No. 5,552,390, Sep. 3, 1996, Scholar, et al., “Phosphorothioate Inhibitors of Metastatic Breast Cancer”; U.S. Pat. No. 5,519,120, May 21, 1996, Dano, et al., “Urokinase-type Plasminogen Activator Receptor Antibodies”; U.S. Pat. No. 5,902,812, May 11, 1999, Brocchini, et al., “Pharmaceutical Piperazine Compounds”; U.S. Pat. No. 5,891,877, Apr. 6, 1999, Brocchini, et al., “Pharmaceutical Compounds”; U.S. Pat. No. 5,750,530, May 12, 1998, Bryans, et al., “Pharmaceutical Diketopiperazine Compounds”; U.S. Pat. No. 5,700,804, Dec. 23, 1997, Collins, et al., “Pharmaceutical Compounds”; U.S. Pat. No. 5,550,213, Aug. 27, 1996, Anderson, et al., “Inhibitors of Urokinase Plasminogen Activator”; U.S. Pat. No. 5,314,994, May 24, 1994, Loskutoff, et al., “Inhibitor of Tissue-type and Urokinase-type Plasminogen Activators”; U.S. Pat. No. 5,340,833, Aug. 23, 1994, Bridges, et al., “Urokinase Inhibitors”, the contents of which are incorporated herein by reference in their entirety.

An especially potent class of reversible urokinase inhibitors is naphthamidines, which are active inhibitors at nanomolar levels. The following references relate to this subject matter: Nienaber V. L., et al., “Structure-Directed Discovery of Potent Non-Peptidic Inhibitors of Human Urokinase that Access a Novel Binding Subsite,” Structure Fold Des, 8(5):553-563 (2000), the contents of which are incorporated herein by reference in their entirety.

A preferred embodiment of the present invention is a compound ET with a targeting ligand comprised of a structure that binds to urokinase.

In preferred embodiments (TL16, TL17), the targeting ligand comprises the following structure:

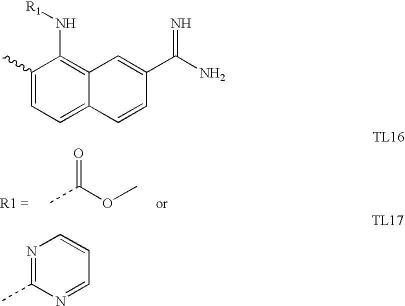

wherein the wavy line is the site of linker attachment to the remainder of the drug complex and the dotted line is the site of attachment of R₁.

Another preferred embodiment is based on the ability of phenylguanidines to inhibit urokinase. The following references relate to this subject matter: Sperl S., et al., “(4-Aminomethyl)Phenylguanidine Derivatives as Nonpeptidic Highly Selective Inhibitors of Human Urokinase,” Proc Natl Acad Sci USA, 97(10):5113-5118 (2000), the contents of which is incorporated herein by reference in its entirety.

In preferred embodiment (TL19), the targeting ligand comprises the following structure:

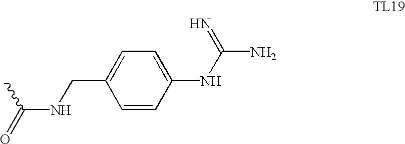

wherein the wavy line is the site of linker attachment to the remainder of the drug complex. In a preferred embodiment, ET has two groups of the structure shown above.

Another class of urokinase selective ligands is based on arginine aldehyde derivatives, which bind reversibly to urokinase with nanomolar affinity. The following references relate to this subject matter: Tamura S. Y., et al., “Synthesis and Biological Activity of Peptidyl Aldehyde Urokinase Inhibitors,” Bioorg Med Chem Lett, 10:983-987 (2000), the contents of which is incorporated herein by reference in its entirety.

In preferred embodiment (TL20 and TL21), the targeting ligand comprises the following structure:

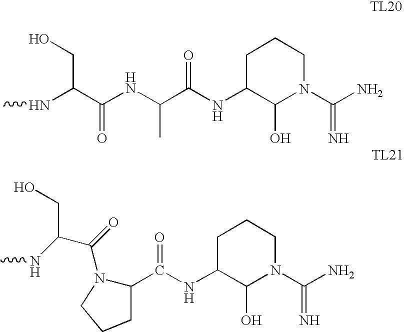

wherein the wavy line is the site of linker attachment to the remainder of the drug complex, and the serine residue has the D-configuration and the remainder of the amino acid residues has the L-configuration; or wherein the structures are L, D, or a racemic mixture.

Additional urokinase binding ligands are described in the neoantigen section that also comprise targeting ligands for urokinase.

Plasmin Selective Ligands:

As discussed above, many types of malignancies are characterized by high levels of urokinase and tissue plasminogen activator, which converts plasminogen into plasmin. Adenocarcinoma cells of the breast, colon and malignant osteoscarcoma bind large quantities of plasminogen and plasmin on the cell surface (10⁵ to 5×10⁷ molecules/cell). The membrane binding facilitates activation of the plasminogen into plasmin. Plasmin binds approximately 80 times tighter than plasminogen. In addition, membrane bound plasmin is resistant to inactivation by α₂ antiplasmin and α₂-macroglobulin. Cytokeratin 8 is the major plasminogen receptor on carcinoma cells. Plasminogen also binds to cell surface annexin II on tumors. Plasminogen is widely distributed throughout the body with plasma concentrations of approximately 1-2 micromolar. However, proteolytically active plasmin is tightly regulated and inhibited by a variety of naturally occurring protease inhibitors. Plasmin plays an important physiological role in fibrinolysis, wound healing, and ovulation. Congenital deficiency of plasminogen is characterized by the development of fibrinous conjunctivitis. The following references relate to this subject matter: Hembrough T A, et al., “A Cytokeratin 8-Like Protein with Plasminogen-Binding Activity is Present on the External Surfaces of Hepatocytes, HepG2 Cells and Breast Carcinoma Cell Lines,” J Cell Sci., 108 (Pt 3):1071-82 (1995); Campbell P G, et al., “Binding and Activation of Plasminogen on the Surface of Osteosarcoma Cells,” J Cell Physiol, 159(1):1-10 (1994); Hembrough T A, et al., “Cell-Surface Cytokeratin 8 is the Major Plasminogen Receptor on Breast Cancer Cells and is Required for the Accelerated Activation of Cell-Associated Plasminogen by Tissue-Type Plasminogen Activator,” J Biol Chem, 271 (41): 25684-91 (1996); Hembrough TA, et al., “Cytokeratin 8 Released by Breast Carcinoma Cells In Vitro Binds Plasminogen and Tissue-Type Plasminogen Activator and Promotes Plasminogen Activation,” Biochem J., 317(Pt 3): 763-9 (1996); Clavel C., et al., “Detection of The Plasmin System in Human Mammary Pathology Using Immunofluorescence,” Cancer Res., 46(11):5743-7 (1986); Ranson M., et al., “Increased Plasminogen Binding is Associated with Metastatic Breast Cancer Cells: Differential Expression of Plasminogen Binding Proteins,” Br J Cancer, 77(10):1586-97 (1998); Costantini V., et al., “Occurrence of Components of Fibrinolysis Pathways In Situ in Neoplastic and Nonneoplastic Human Breast Tissue,” Cancer Res, 51(1):354-8 (1991); Gonzalez-Gronow M., et al., “Plasmin Binding to the Plasminogen Receptor Enhances Catalytic Efficiency and Activates the Receptor for Subsequent Ligand Binding,” Arch Biochem Biophys, 286(2):625-8 (1991); Burtin P; Fondaneche M C., “Receptor for plasmin on human carcinoma cells,” J Natl Cancer Inst, 80(10): 762-5 (1988); Miles LA, et al., “Role of Cell-Surface Lysines in Plasminogen Binding to Cells: Identification of Alpha-Enolase as a Candidate Plasminogen Receptor,” Biochemistry, 30(6):1682-91 (1991); Burtin P., et al., “The Plasmin System in Human Adenocarcinomas and their Metastases. A Comparative Immunofluorescence Study,” Int J Cancer, 39(2):170-8 (1987); Burtin P., et al., “The Plasmin System in Human Colonic Tumors: An Immunofluorescence Study,” Int J Cancer, 35(3):307-14 (1985); Plow E F, et al., “The Plasminogen System and Cell Surfaces: Evidence for Plasminogen and Urokinase Receptors on the Same Cell Type,” J Cell Biol, 103(6 Pt 1):2411-20 (1986); Kwaan HC, “The Plasminogen-Plasmin System in Malignancy,” Cancer Metastasis Rev, 11(3-4):291-311 (1992); Correc P., et al., “The Presence of Plasmin Receptors on Three Mammary Carcinoma MCF-7 Sublines.,” Int J Cancer, 46(4):745-50 (1990); Correc P., et al., “Visualization of the Plasmin Receptor on Carcinoma Cells,” Int J Cancer, 50(5):767-71 (1992), the contents of which are incorporated herein by reference in their entirety.

There have been attempts to utilize the enhanced plasmin activity of tumor cells to activate plasmin selective cytotoxic prodrugs. Plasminogen activators have been employed to target malignant cells by coupling cytotoxic agents to the protein plasminogen activator inhibitor types 1 and 2. However, tumor-associated plasmin has not been utilized as a target site for the selective delivery of anti-cancer drugs. The following references relate to this subject matter: Chakravarty P K, et al., “Plasmin-Activated Prodrugs for Cancer Chemotherapy. 1. Synthesis and Biological Activity of Peptidylacivicin and Peptidylphenylenediamine Mustard,” J Med Chem, 26(5):633-8 (1983); Chakravarty P K, et al., “Plasmin-Activated Prodrugs for Cancer Chemotherapy. 2. Synthesis and Biological Activity of Peptidyl Derivatives of Doxorubicin,” J Med Chem, 26(5):638-44 (1983); Abaza M S, et al., “Anti-Urokinase-Type Plasminogen Activator Monoclonal Antibodies Inhibit the Proliferation of Human Breast Cancer Cell Lines In Vitro,” Tumour Biol, 19(4):229-37 (1998); Towle M J, et al., “Inhibition of Urokinase by 4-Substituted.”; U.S. Pat. No. 5,679,350, Oct. 21, 1997, Jankun, et al., “Method of Delivery of a Medicament to a Cancer Cell using a Pathway of Plasminogen Activator Material.”, the contents of which are incorporated herein by reference in their entirety.

Plasmin is serine protease with broad substrate specificity for cleaving amide bond adjacent to lysine or arginine. P-amidinophenol esters are potent inhibitors of plasmin. These derivatives are inverse substrates and acylate a serine hydroxy group in the active site of the enzyme. If the acyl enzyme intermediate is sufficiently stable, irreversible inactivation of enzyme activity results. The p-amidinophenol ester of p-methoxybenzoic acid irreversibly inactivates plasmin. The following references relate to this subject matter: Nozawa M., et al., “Behavior of Trypsin and Related Enzymes Toward Amidinophenyl Esters,” J Pharmacobiodyn, 4(8):559-64 (1981); Nozawa M., et al., “Comparative Studies on the Structure of Active Sites. Behavior of “Inverse Substrates” Toward Trypsin and Related Enzymes,” J Biochem (Tokyo), 91(6):1837-43 (1982); Yamada H., et al., “Differentiation of Tryptic Enzymes Based on Enantiomeric Specificity at the Deacylation Step,” FEBS Lett, 227(2):195-7 (1988); McLaren AB; Tanizawa K., “Deacylation Rate Constants of Acylated Human and Porcine Plasmins,” Aust. J. Biol. Sci., 37:205-10 (1984); Tanizawa K., et al., ““Inverse Substrates” for Trypsin. Efficient Enzymatic Hydrolysis of Certain Esters with a Cationic Center in the Leaving Group,” J Amer. Chem Soc., 99(13):4485-4488 (1977); Turner A D, et al., “p-Amidino Esters as Irreversible Inhibitors of Factors IXa and Xa and Thrombin,” Biochemistry, 25:4929-4935 (1986); Fujioka T., et al., “Analysis of Latent Properties of Trypsin. Acyl Trypsins Derived from Enantiomeric Pairs of “Inverse Substrates”,” J Biochem., 89:637-643 (1981); Lynas J., et al., “Peptidyl Inverse Esters of p-Methoxybenzoic Acid: A Novel Class of Potent Inactivator of the Serine Proteases,” Biochem J., 325 (Pt 3):609-16 (1997); Fujioka T., et al., “Analysis of Latent Properties of Trypsin. Acyl Trypsins Derived from Enantiomeric Pairs of “Inverse Substrates””, J Biochem (Tokyo), 89(2):637-43 (1981), the contents of which are incorporated herein by reference in their entirety.

By attaching a linker to this class of inhibitors, it is possible to chemically couple or target molecules to plasmin. This serves as the basis for being able to employ plasmin as a targeting entity.

A preferred embodiment of the present invention is a compound ET with a targeting ligand comprised of a structure that binds to plasmin.

In preferred embodiment (TL22), the targeting ligand comprises the following structure:

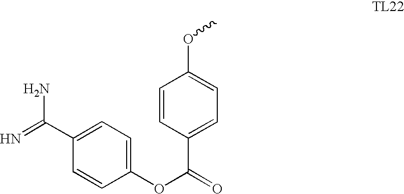

wherein the wavy line is the site of the linker attachment to the remainder of the drug. Other preferred plasmin binding ligands are described in the neoantigen section that can be targeting ligands.

Cathepsin B Targeting Ligands

Cathepsin B is over-expressed and a major adverse prognostic factor in many human malignancies. Cathepsin B binds to annexin II on the surface of tumor cells. The following reference relate to this subject matter: Mai J., et al., “Cell Surface Complex of Cathepsin B/Annexin II Tetramer in Malignant Progression,”

Biochimica Biophysica Acta (BBA)-Protein Molecular Enzymology, 1477(1-2):215-230 (2000), the contents of which is incorporated herein by reference in its entirety.

A preferred embodiment of the present invention is a compound ET with a targeting ligand comprised of a structure that binds to Cathepsin B. This is discussed in more detail in the neoantigen section.

Matripase, Seprase, and Fibroblast Activation Protein Targeting Ligands

A preferred embodiment of the present invention is a compound ET with a targeting ligand comprised of a structure that binds to seprase, or fibroblast activation protein. This is discussed in more detail in the neoantigen section.

Prostate Specific Membrane Antigen Targeting Ligands:

Prostatic adenocarcinoma cells have high concentrations of the enzyme Glutamate Carboxypeptidase II or Prostatic Specific Membrane Antigen (PSMA) on the cell surface. In addition, the enzyme is present on the brush border of the kidneys, the luminal surface of parts of the proximal small intestine and in the brain. Radiolabelled monoclonal antibodies against PSMA (ProstaScint™) are in clinical use to assess metastatic tumor spread. PSMA has also been detected on the surface of tumor neovasculature. Inhibitors of PSMA have been described as anti-cancer drugs. However, the activity of these compounds is weak and unlikely to be of clinical utility. PSMA positive human prostate tumor cells readily grow in vitro in the presence of high concentrations of 2-(phosphonomethyl)pentanedioic acid, a potent inhibitor of PSMA (A. Glazier, unpublished observations). Efforts are also underway to utilize PSMA related peptides as vaccines against prostate cancer. The following references relate to this subject matter: U.S. Pat. No. 5,804,602, Sep. 8, 1998, Slusher, et al., “Methods of Cancer Treatment Using NAALADase Inhibitors”; U.S. Pat. No. 5,795,877, Aug. 18, 1998, Jackson, et al., “Inhibitors of NAALADase Enzyme Activity”; Murphy G P, et al., “Current Evaluation of the Tissue Localization and Diagnostic Utility of Prostate Specific Membrane Antigen,” Cancer, 83(11):2259-69 (1998); Heston W D, “Characterization and Glutamyl Preferring Carboxypeptidase Function of Prostate Specific Membrane Antigen: A Novel Folate Hydrolase,” Urology, 49(3A Suppl):104-12 (1997); Tiffany C W, et al., “Characterization of the Enzymatic of PSM: Comparison with Brain NAALADase [In Process Citation],” Prostate, 39(1):28-35 (1999); Murphy G P, et al., “Comparison of Serum PSMA, PSA Levels with Results of Cytogen-356 Prostascint Scanning in Prostatic Cancer Patients,” Prostate, 33(4):281-5 (1997); Serval V. et al., “Competitive Inhibition of N-Acetylated-Alpha-Linked Acidic Dipeptidase Activity by N-Acetyl-L-Aspartyl-Beta-Linked L-Glutamate,” J Neurochem, 55(1):39-46 (1990); Liu H. et al., “Constitutive and Antibody-Induced Internalization of Prostate-Specific Membrane Antigen,” Cancer Res, 58(18):4055-60 (1998); Murphy G P, et al., “Current Evaluation of the Tissue Localization and Diagnostic Utility of Prostate Specific Membrane Antigen,” Cancer, 83(11):2259-69 (1998); Jackson P F, et al., “Design, Synthesis, and Biological Activity of a Potent Inhibitor of the Neuropeptidase N-Acetylated Alpha-Linked Acidic Dipeptidase,” J Med Chem, 39(2):619-22 (1996); Troyer J K; Beckett M L; Wright G L Jr., “Detection and Characterization of the Prostate-Specific Membrane Antigen (PSMA) in Tissue Extracts and Body Fluid,” Int J Cancer, 62(5):552-8 (1995); Douglas T H, et al., “Effect of Exogenous Testosterone Replacement on Prostate-Specific Antigen and Prostate-Specific Membrane Antigen Levels in Hypogonadal Men,” J Surg Oncol, 59(4):246-50 (1995); Kawakami M; Nakayama J., “Enhanced Expression of Prostate-Specific Membrane Antigen Gene in Prostate Cancer as Revealed by in Situ Hybridization,” Cancer Res, 57(12):23214 (1997); Israeli R S, et al., “Expression of the Prostate-Specific Membrane Antigen,” Cancer Res, 54(7):1807-11 (1994); Halsted C H, et al., “Folylpoly-Gamma-Glutamate Carboxypeptidase from Pig Jejunum. Molecular Characterization and Relation to Glutamate Carboxypeptidase II,” J Biol Chem, 273(32):20417-24 (1998); Luthi-Carter R. et al., “Hydrolysis of The Neuropeptide N-Acetylaspartylglutamate (NAAG) by Cloned Human Glutamate Carboxypeptidase II,” Brain Res, 795(1-2):341-8 (1998); Grauer L S, et al., “Identification, Purification, and Subcellular Localization of Prostate- Specific Membrane Antigen PSM' Protein in the LNCaP Prostatic Carcinoma Cell Line,” Cancer Res, 58(21):4787-9 (1998); Slusher B S; Tsai G; Yoo G; Coyle J T, “Immunocytochemical Localization of the N-Acetyl-Aspartyl-Glutamate (NAAG) Hydrolyzing Enzyme N-Acetylated Alpha-Linked Acidic Dipeptidase (Naaladase),” J Comp Neurol, 315(2):217-29 (1992); Luthi-Carter R. et al., “Isolation and Expression of a Rat Brain cDNA Encoding Glutamate Carboxypeptidase II,” Proc NatlAcad Sci, 95(6):3215-20 (1998); Troyer J. K., et al., “Location of Prostate-Specific Membrane Antigen in the LNCaP Prostate Carcinoma Cell Line,” Prostate, 30(4):232-42 (1997); Sweat S. D, et al., “Prostate-Specific Membrane Antigen Expression is Greatest in Prostate Adenocarcinoma and Lymph Node Metastases,” Urology, 52(4):637-40 (1998); Luthi-Carter R., et al., “Molecular Characterization of Human Brain N-Acetylated Alpha-Linked Acidic Dipeptidase (Naaladase),” J Pharmacol Exp Ther, 286(2):1020-5 (1998); Bzdega T. et al., “Molecular Cloning of a Peptidase Against N-Acetylaspartylglutamate from a Rat Hippocampal cDNA Library,” J Neurochem, 69(6):2270-7 (1997); Liu H. et al., “Monoclonal Antibodies to the Extracellular Domain of Prostate-Specific Membrane Antigen also React with Tumor Vascular Endothelium,” Cancer Res, 57(17):3629-34 (1997); Barren R J 3^{rd}. et al., “Monoclonal Antibody 7E11.C5 Staining of Viable LNCaP Cells,” Prostate, 30(1):65-8 (1997); Kawamata H. et al., “Active-MMP2 in Cancer Cell Nests of Oral Cancer Patients: Correlation with Lymph Node Metastasis,” Int J Oncol, 13(4):699-704 (1998); Weissensteiner T., “Prostate Cancer Cells Show a Nearly 100-Fold Increase in the Expression of the Longer of Two Alternatively Spliced mRNAs of the Prostate- Specific Membrane Antigen (PSM) [letter],” Nucleic Acids Res, 26(2):687 (1998); Bostwick D G, et al., “Prostate Specific Membrane Antigen Expression in Prostatic Intraepithelial Neoplasia and Adenocarcinoma: A Study of 184 Cases,” Cancer, 82(11):2256-61 (1998); Pinto JT, et al., “Prostate-Specific Membrane Antigen: A Novel Folate Hydrolase in Human Prostatic Carcinoma Cells,” Clin Cancer Res, 2(9):1445-51 (1996); Silver DA, et al., “Prostate-Specific Membrane Antigen Expression in Normal and Malignant Human Tissues,” Clin Cancer Res, 3(1):81-5 (1997); Sweat S D, et al., “Prostate-Specific Membrane Antigen Expression is Greatest in Prostate Adenocarcinoma and Lymph Node Metastases,” Urology, 52(4):637-40 (1998); Carter R. E., et al., “Prostate-Specific Membrane Antigen is a Hydrolase with Substrate and Pharmacologic Characteristics of a Neuropeptidase,” Proc Natl Acad Sci, 93(2):749-53 (1996); Fair W. R.; et al., “Prostate-Specific Membrane Antigen,” Prostate, 32(2):140-8 (1997); Slusher B S, et al., “Rat Brain N-Acetylated Alpha-Linked Acidic Dipeptidase Activity. Purification and Immunologic Characterization,” J Biol Chem, 265(34):21297-301 (1990); Subasinghe N. et al., “Synthesis Of Acyclic And Dehydroaspartic Acid Analogues of Ac-Asp-Glu- OH and Their Inhibition of Rat Brain N-acetylated Alpha-linked Acidic Dipeptidase (NAALA Dipeptidase),” J Med Chem, 33(10):2734-44 (1990); Stauch B L, et al., “The Effects of N-acetylated Alpha-linked Acidic Dipeptidase (NMLADase) Inhibitors on [3H]NAAG Catabolism In Vivo,” Neurosci Lett, 100(1-3):295-300 (1989); Berger U. V. et al., “The Immunocytochemical Localization of N-acetylaspartyl glutamate, its Hydrolysing Enzyme NAALADase, and the NMDAR-1 Receptor at a Vertebrate Neuromuscular Junction,” Neuroscience, 64(4):847-50 (1995); Wright GL Jr. et al., “Upregulation of Prostate-Specific Membrane Antigen after Androgen- Deprivation Therapy,” Urology, (2):326-34 (1996); Chang S. S., et al., “Prostate-specific Membrane Antigen is Produced in Tumor-associated Neovasculature,” Clin Cancer Res, 5:2674-2681 (1999). Tjoa B. A., et al., “Follow-Up Evaluation of a Phase II Prostate Cancer Vaccine Trial,” Prostate, 40(2):125-9 (1999), the contents of which are incorporated herein by reference in their entirety.

PSMA is a zinc carboxypeptidase, which catalyzes the hydrolysis of N-acetyl-aspartylglutamate and gamma glutamates. The enzyme is potently inhibited by phosphorous based transition state analogs. 2-(phosphonomethyl)-pentanedioic acid inhibits the enzyme with a Ki of 0.3 nanomolar. As described later in Example 1, it is possible to attach a linker to compounds of this class and retain inhibitory and enzyme binding capacity.

A preferred embodiment of the present invention is a compound ET with a targeting ligand comprised of a structure that binds to PSMA. In a preferred embodment, the targeting ligand comprises the following structure:

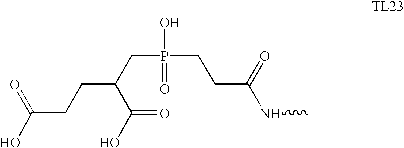

wherein the wavy line is the site of linker attachment to the remainder of the drug complex.

The scope of the present invention also encompasses a ligand-linker complex that binds to PSMA and the use of this ligand-linker complex to target drugs to PSMA positive cells wherein the ligand is not a monoclonal antibody. The present invention also includes an anti-cancer drug comprised of the structure shown above covalently linked to a cytotoxic drug or cytotoxic agent.

Sigma Receptor Targeting Ligands

Sigma receptors are a class of membrane associated receptors, that are present in increased amounts on a variety of malignant tumors including: prostatic adenocarcinoma, neuroblastoma, melanoma, breast carcinoma, pheochromocytoma, renal carcinoma, colon carcinoma, and lung carcinoma. Prostatic adenocarcinoma cells have approximately 2 million receptors molecules/cells. Sigma receptors are also present on a variety of normal tissues including the liver, brain, kidney, and endocrine glands. Radiolabelled sigma receptor ligands concentrate in malignant tumors in vivo and have been described as tumor imaging agents. However, sigma receptors have not previously been exploited for targeting antineoplastic drugs. By themselves, sigma receptors are unlikely to have sufficient tumor selectivity for tumor targeting purposes. However, sigma receptor targeting by a multifunctional delivery vehicle, which jointly targets other receptors enriched on tumor cells, can provide excellent tumor specificity. The following references relate to this subject matter: John C S, et al., “99mTc-labeled Sigma-Receptor-Binding Complex: Synthesis, Characterizaton, and Specific Binding to Human Ductal Breast Carcinoma (T47D) Cells,” Bioconjug Chem, 8(3):304-9 (1997); John C S, et al., “A Malignant Melanoma Imaging Agent: Synthesis, Characterization, In Vitro Binding and Biodistribution of Iodine-125-(2-piperidinylaminoethyl)4-iodobenzamide,” J Nucl Med, 34(12):2169-75 (1993); John C S, et al., “Synthesis, In Vitro Binding, and Tissue Distribution of Radioiodinated 2-[125I]N-(N-benzylpiperidin-4-yl)-2-iodo benzamide, 2-[125I]BP: A Potential Sigma Receptor Marker for Human Prostate Tumors,” Nucl Med Biol, 25(3):189-94 (1998); John C S, et al., “An Improved Synthesis of [125I]N-(diethylaminoethyl)-4-iodobenzamide: A Potential Ligand for Imaging Malignant Melanoma,” Nucl Med Biol, 20(1):75-9 (1993); Zhang Y. et al., “Characterization of Novel N,N′-disubstituted Piperazines as Sigma Receptor Ligands,” J Med Chem, 41(25):4950-7 (1998); Vilner B J, et al. “Cytotoxic Effects of Sigma Ligands: Sigma Receptor-Mediated Alterations in Cellular Morphology and Viability,” J Neurosci, 15(1 Pt 1):117-34 (1995); Waterhouse R N, et al., “Examination of Four 123I-labeled Piperidine-Based Sigma Receptor Ligands as Potential Melanoma Imaging Agents: Initial Studies in Mouse Tumor Models,” Nucl Med Biol, 24(6):587-93 (1997); John C S, et al., “Synthesis and Pharmacological Characterization of 4-[125I]-N-(N-benzylpiperidin-4-yl)-4-iodobenzamide: A High Affinity Sigma Receptor Ligand for Potential Imaging of Breast Cancer,” Cancer Res, 55(14):3022-7 (1995); Zamora PO; Moody TW; John CS, “Increased Binding to Sigma Sites of N-[1′(2-piperidinyl)ethyl)-4-[I-125]-iodobenzamide (I-125-PAB) With Onset of Tumor Cell Proliferation,” Life Sci, 63(18):1611-8 (1998); Bern W T, et al., “Over-expression of Sigma Receptors in Nonneural Human Tumors,” Cancer Res, 51(24):6558-6 (1991); Mach R H, et al. “Sigma 2 Receptors as Potential Biomarkers of Proliferation in Breast Cancer,” Cancer Res, 57(1):156-61 (1997); Brent P J, et al. “Sigma Binding Site Ligands Inhibit Cell Proliferation in Mammary and Colon Carcinoma Cell Lines and Melanoma Cells in Culture,” Eur J Pharmacol, 278(2):151-60 (1995); John C S, et al., “Sigma Receptors are Expressed in Human Non-Small Cell Lung Carcinoma,” Life Sci, 56(26):2385-92 (1995); Vilner B J, et al., “Sigma-I and Sigma-2 Receptors are Expressed in a Wide Variety of Human and Rodent Tumor Cell Lines.,” Cancer ReS, 55(2):408-13 (1995); Everaert H, et al., “Sigma-Receptor Imaging by Means of I123-IDAB Scintigraphy: Clinical Application in Melanoma and Non-Small Cell Lung Cancer,” Anti-cancer Res, 17(3B):1577-82 (1997); Glennon R A, et al., “Structural Features Important for Sigma 1 Receptor Binding,” J Med Chem, 37(8):1214-9 (1994); John C S, et al., “Substituted Halogenated Arylsulfonamides: A New Class Of Sigma Receptor Binding Tumor Imaging Agents,” J Med Chem, 41(14):2445-50 (1998); John C S, et al., “Synthesis and Characterization of [125I]-N-(N-benzylpiperidin-4-yl)-4- iodobenzamide, a New Sigma Receptor Radiopharmaceutical High-affinity Binding to MCF-7 Breast Tumor Cells,” J Med Chem, 37(12):1737-9 (1994); Dence C S, John C S, Bowen W D, Welch M J, “Synthesis and Evaluation of [18F] Labeled Benzamides: High Affinity Sigma Receptor Ligands for PET Imaging,” Nucl Med Biol, 24(4):333-40 (1997); de Costa B R, et al., “Synthesis and Evaluation of Optically Pure [3H]-(+)-pentazocine, A Highly Potent and Selective Radioligand for Sigma Receptors,” FEBS Lett, 251(1-2):53-8 (1989); Waterhouse RN, Mardon K, O'Brien J C, “Synthesis and Preliminary Evaluation of [123I]1-(4-cyanobenzyl)-4-[[(trans-iodopropen-2-yl)oxy]methyl]piperidin e: A Novel High Affinity Sigma Receptor Radioligand for SPECT,” Nucl Med Biol, 24(1):45-5 (1997); John C S, et al., “Synthesis, In Vitro Pharmacologic Characterization, and Preclinical Evaluation of N-[2-(1′-piperidinyl)ethyl]-3-[125I]iodo-4-methoxybenzamide (P[125I]MBA) for Imaging Breast Cancer,” Nucl Med Biol, 26(4):377-82 (1999); John C S, et al., “Synthesis, In Vitro Validation and In Vivo Pharmacokinetics of [125I]N-[2-(4-iodophenyl)ethyl]-N-methyl-2-(1-piperidinyl) ethylamine: A High-Affinity Ligand for Imaging Sigma Receptor Positive Tumors,” Nucl Med Biol, 23(6):761-6 (1996); Huang Y., et al., “Synthesis and Quantitative Structure-activity Relationships of N-(1-benzylpiperidin-4-yl)phenylacetamides and Related Analogues as Potent and Selective Sigmal Receptor Ligands,” J Med Chem, 41(13):2361-70 (1998); Berardi F., et al., “N-[omega-(Tetralin-1-yl)alkyl] Derivatives of 3,3-Dimethylpiperidine are Highly Potent and Selective Sigmal or Sigma2 Ligands,” J Med Chem, 41(21):3940-7 (1998), the contents of which are incorporated herein by reference in their entirety.

A preferred embodiment of the present invention is a compound ET with a targeting ligand comprised of a structure that binds to sigma receptors.

A large variety of lipophilic piperazines are known to bind with high affinity to sigma receptors. The following reference relates to this subject matter: Zhang Y. et al., “Characterization of Novel N,N′-disubstituted Piperazines as Sigma Receptor Ligands,” J Med Chem, 41(25):4950-7 (1998), the contents of which is incorporated herein by reference in its entirety.

In preferred embodiments (TL24 and TL25) the targeting ligands comprise the following structures:

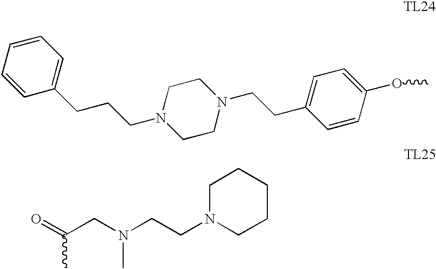

wherein the wavy line is the site of linker attachment to the remainder of the drug complex.

The following reference relates to this subject matter: John C S, et al., “99m Tc-labeled Sigma-Receptor-Binding Complex: Synthesis, Characterization, and Specific Binding to Human Ductal Breast Carcinoma (T47D) Cells,” Bioconjug Chem, 8(3):304-9 (1997), the contents of which is incorporated herein by reference in its entirety.

Nucleoside Transporter Targeting Ligands

Nucleoside Transporter (NT) catalyzes the equilibrative transport of nucleosides into cells. The transporter is markedly over-expressed in malignant cells following exposure to agents that interfere with the denovo synthesis of nucleosides derivatives. For example, human bladder cancer cells treated with an inhibitor, to a thymidylate synthase inhibitor displayed a 39 times increase in the amount of nucleoside transporter protein. Potent inhibitors of NT include the drugs dipyridamole and Dilazep. In addition, nitrobenzylthioadenosine analogs are potent inhibitors. The following references relate to this subject matter: Griffiths M., et al., “Cloning of a Human Nucleoside Transporter Implicated in the Cellular Uptake of Adenosine and Chemotherapeutic Drugs,” Nat Med, 3(1):89-93 (1997); Pressacco J., et al., “Effects of Thymidylate Synthase Inhibition on Thymidine Kinase Activity and Nucleoside Transporter Expression,” Cancer Res, 55(7):1505-8 (1995); Belt J. A., et al., “Nucleoside Transport in Normal and Neoplastic Cells,” Adv Enzyme Regul, 33:235-52 (1993); Wiley J. S., et al., “A New Fluorescent Probe for the Equilibrative Inhibitor-Sensitive Nucleoside Transporter. 5′-S-(2-Aminoethyl)-N6-(4-Nitrobenzyl)-5′-Thioadenosine (SAENTA)-chi 2-Fluorescein,” Biochem J, 273(Pt 3):667-72 (1991); Agbanyo F. R., et al., “5′-S-(2-Aminoethyl)-N6-(4-Nitrobenzyl)-5′-Thioadenosine (SAENTA), a Novel Ligand with High Affinity for Polypeptides Associated with Nucleoside Transport. Partial Purification of the Nitrobenzylthioinosine-Binding Protein of Pig Erythrocytes by Affinity Chromatography,” Biochem J, 270(3):605-14 (1990); Baldwin S. A., et al., “Nucleoside Transporters: Molecular Biology and Implications for Therapeutic Development,” Mol Med Today, 5:216-224 (1999), the contents of which are incorporated herein by reference in their entirety.

A preferred embodiment of the present invention is a compound ET with a targeting ligand comprised of a structure that binds to nucleoside transporter proteins. In preferred embodiments (TL26, TKL27, TL28 and TL29)) the targeting ligands comprise the following structures:

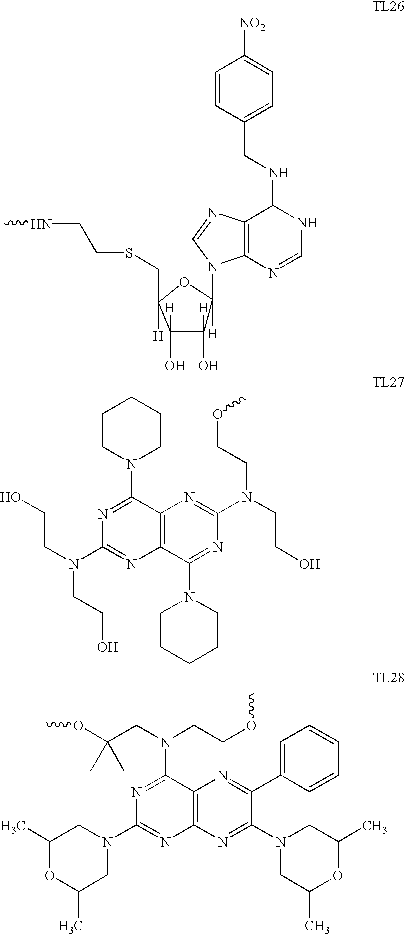

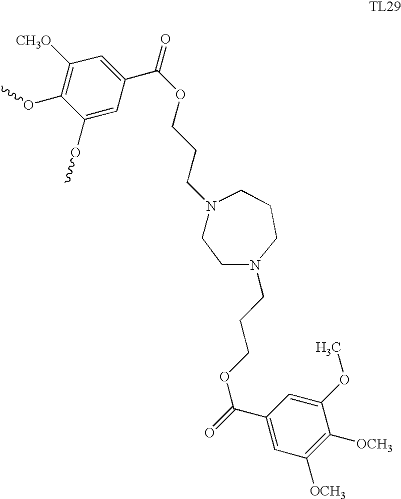

wherein the wavy line is the site of linker attachment to the remainder of the drug ET or H.

Folate Receptor Targeted Ligands

The high affinity folate receptor (FR) is over-expressed in a number of malignancies including most ovarian and endometrial carcinomas and some breast, lung, colorectal, and renal cell cancers. Nonmucinous ovarian carcinomas have levels of FR that are increased 80 to 90 times over the levels present in normal ovaries. FR is also widely distributed in normal tissues with high levels in normal kidney, lung, thyroid, and choroidal plexus. Substantial efforts have been directed towards targeting folate receptors for diagnosis and therapy of FR+ malignancies. Monoclonal antibodies, conjugates of folic acid and radiolabelled groups, conjugates of folic acid and cytotoxic agents, and cancer vaccines have all been explored. A major barrier to success is the high concentration of FR in vital locations such as the kidney and choroid plexus. In rats radio-imaging studies have demonstrated intense accumulation in normal kidneys of FR targeted compounds. The following references relate to this subject matter: Susten S S, et al., “A Fluorescent Analogue of Methotrexate as a Probe for Folate Antagonist Molecular Receptors,” Biochem Pharmacol, 33(12):1957-62 (1984); Holm J., et al., “A High-Affinity Folate Binding Protein in Proximal Tubule Cells of Human Kidney,” Kidney Int, 41(1):50-5 (1992); Kranz DM, et al., “Conjugates of Folate and Anti-T-Cell-Receptor Antibodies Specifically Target Folate-Receptor-Positive Tumor Cells for Lysis,” Proc Natl Acad Sci USA, 92(20):9057-61 (1995); Westerhof G R, et al., “Carrier- and Receptor-Mediated Transport of Folate Antagonists Targeting Folate-Dependent Enzymes: Correlates of Molecular-Structure and Biological Activity,” Mol Pharmacol, 48(3):459-71 (1995); Wang S., et al., “Design and Synthesis of [111In]DTPA-Folate for use as a Tumor-Targeted Radiopharmaceutical,” Bioconjug Chem, 8(5):673-9 (1997); Rothberg K G, et al., “The Glycophospholipid-Linked Folate Receptor Internalizes Folate Without Entering the Clathrin-Coated Pit Endocytic Pathway,” J Cell Biol, 110(3):637-49 (1990); Fan J., et al., “Novel Substrate Analogs Delineate an Endocytotic Mechanism for Uptake of Folate via the High-Affinity, Glycosylphosphatidylinositol-Linked Transport Protein in L1210 Mouse Leukemia Cells,” Oncol Res, 7(10-11):511-6 (1995); Mantovani L T, et al., “Folate Binding Protein Distribution in Normal Tissues and Biological Fluids from Ovarian Carcinoma Patients as Detected by the Monoclonal Antibodies MOv18and MOv19,” Eur J Cancer, 30A(3):363-9 (1994); Leamon CP; Low PS, “Membrane Folate-Binding Proteins are Responsible for Folate-Protein Conjugate Endocytosis into Cultured Cells,” Biochem J, 291 (Pt 3):855-60 (1993); Holm J, et al., “Folate Receptor of Human Mammary Adenocarcinoma,” APMIS, 102(6):413-9 (1994); Holm J., et al., “Folate Receptors in Malignant and Benign Tissues of Human Female Genital Tract,” Biosci Rep, 17(4):415-27 (1997); Wang S., et al., “Synthesis, Purification, and Tumor Cell Uptake of 67Ga-deferoxamine—Folate, a Potential Radiopharmaceutical for Tumor Imaging,” Bioconjug Chem, 7(1):56-62 (1996); Mathias C J, et al., “Tumor-Selective Radiopharmaceutical Targeting via Receptor-Mediated Endocytosis of Gallium-67-Deferoxamine-Folate,” J Nucl Med, 37(6):1003-8 (1996); Ladino C A, et al., “Folate-Cantansinoids: Target-Selective Drugs of Low Molecular Weight,” Int J Cancer, 73(6):859-64 (1997); Li S; Deshmukh HM; Huang L., “Folate-Mediated Targeting of Antisense Oligodeoxynucleotides to Ovarian Cancer Cells,” Pharm Res, 15(10):1540-5 (1998); Reddy JA, Low PS, “Folate-Mediated Targeting of Therapeutic and Imaging Agents to Cancers,” Crit Rev Ther Drug Carrier Syst, 15(6):587-627 (1998); Leamon CP; Low PS, “Membrane Folate-Binding Proteins are Responsible for Folate-Protein Conjugate Endocytosis into Cultured Cells,” Biochem J, 291 (Pt 3):855-60 (1993); Leamon C P; Low P S, “Delivery of Macromolecules into Living Cells: a Method that Exploits Folate Receptor Endocytosis,” Proc Natl Acad Sci USA, 88(13):5572-6 (1991); Ginobbi P., et al., “Folic Acid-Polylysine Carrier Improves Efficacy of c-myc Antisense Oligodeoxynucleotides on Human Melanoma (M14) Cells,” Anti-cancer Res, 17(1A):29-35 (1997); Holm J., et al., “High-Affinity Folate Receptor in Human Ovary, Serous Ovarian Adenocarcinoma, and Ascites: Radioligand Binding Mechanism, Molecular Size, Ionic Properties, Hydrophobic Domain, and Immunoreactivity,” Arch Biochem Biophys, 366(2):183-91 (1999); Reddy J A; Low P S, “Folate-Mediated Targeting of Therapeutic and Imaging Agents to Cancers [In Process Citation],” Crit Rev Ther Drug Carrier Syst, 15(6):587-627 (1998); Li P Y, et al., “Local Concentration of Folate Binding Protein GP38 in Sections of Human Ovarian Carcinoma by In Vitro Quantitative Autoradiography,” J Nucl Med, 37(4):665-72 (1996); Toffoli G., et al., “Over-expression of Folate Binding Protein in Ovarian Cancers,” Int J Cancer, 74(2):193-8 (1997); Birn H; Selhub J; Christensen E I, “Internalization and Intracellular Transport of Folate-Binding Protein in Rat Kidney Proximal Tubule,” Am J Physiol, 264(2 Pt 1):C302-10 (1993); Abraham A., et al., “Folate Analogues. 33. Synthesis of Folate and Antifolate Poly-Gamma- Glutamates by [(9-fluorenylmethoxy)oxy]carbonyl Chemistry and Biological Evaluation of Certain Methotrexate Polyglutamate polylysine Conjugates as Inhibitors of the Growth of H35 Hepatoma Cells,” J Med Chem, 33(2):711-7 (1990); Gabizon A, et al., “Targeting Folate Receptor with Folate Linked to Extremities of Poly(Ethylene Glycol)-Grafted Liposomes: In Vitro Studies,” Bioconjug Chem, 10(2):289-98 (1999); Ladino C A, et al., “Folate-Cantansinoids: Target-Selective Drugs of Low Molecular Weight,” Int J Cancer, 73(6):859-64 (1997); Leamon C P, Low P S, et al., “Selective Targeting of Malignant Cells with Cytotoxin-Folate Conjugates,” J. Drug Target, 2(2):101-1 (1994); Konda S. D., et al., “Development of a Tumor-Targeting MR Contrast Agent using the High-Affinity Folate Receptor: Work In Progress,” Invest Radiol, 35(1):50-7 (2000); Toffoli G., et al., “Expression of Folate Binding Protein as a Prognostic Factor for Response to Platinum-Containing Chemotherapy and Survival in Human Ovarian Cancer,” Int J Cancer, 79(2):121-6 (1998); Lu J. Y., et al., “Folate-Targeted Enzyme Prodrug Cancer Therapy Utilizing Penicillin-V Amidase and a Doxorubicin Prodrug,” J Drug Target, 7(1):43-53 (1999); Sudimack J; Lee R. J., “Targeted Drug Delivery via the Folate Receptor,” Adv Drug Deliv Rev, 41(2):147-162 (2000); Peoples G. E., et al., “Vaccine Implications of Folate Binding Protein, a Novel Cytotoxic T Lymphocyte-Recognized Antigen System in Epithelial Cancers,” Clin Cancer Res, 5(12):4214-23 (1999), the contents of which are incorporated herein by reference in their entirety.

A preferred embodiment of the present invention is a compound ET with a targeting ligand comprised of a structure that binds to the high affinity folate receptor. The multifactorial properties of multifunctional drug delivery vehicles can allow the FR to be exploited as a tumor target without damage to FR+ non-tumor tissues. For example, a drug targeted to ovarian cancer with targeting ligands for FR and MMP-7 and fatty acid synthase can have increased selectivity for ovarian cancer. Adult kidneys lack or have very low levels of both fatty acid synthase and MMP-7.

In preferred embodiments (TL30)) the targeting ligands comprise the following structures:

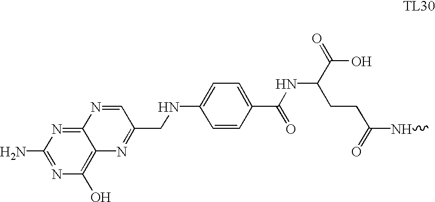

Another preferred embodiment (TL31) shown below is based upon the ability of bicyclic 5-thiapyrimidinones to bind with subpicomolar affinity to the FR. The following references relate to this subject matter: Varney M. D., et al., “Protein Structure-Based Design, Synthesis, and Biological Evaluation of 5-Thia-2,6-diamino-4(3H)-oxopyrimidines: Potent Inhibitors of Glycinamide Ribonucleotide Transformylase with Potent Cell Growth Inhibition,” J Med Chem, 40:2502-2524 (1997), the contents of which are incorporated herein by reference in their entirety.

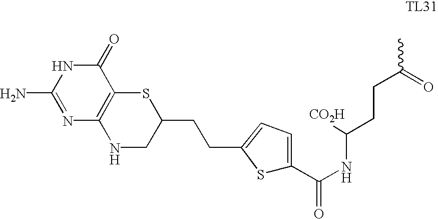

wherein the wavy line is the site of linker attachment to the remainder of the drug complex.

Somatostatin Receptor Targeted Ligands

Somatostatin receptors (SSR) are expressed at high levels in a variety of human malignancies including: breast, prostate, neuroblastoma, medullabalstoma, pancreatic, ovarian, gastrinoma, thyroid, melanoma, renal, lymphoma, glioma, colorectal, small cell lung cancer, and most neuroendocrine tumors. The over-expression of somatostatin receptors on malignant cells has been utilized for both diagnostic and therapeutic purposes. A large variety of radiolabelled SSR analogs have been developed. In addition, conjugates of potent cytotoxic agents have been coupled to SSR binging groups as potential antineoplastic drugs. In addition, a large number of analogs, which bind to SSR, have been investigated as anti-cancer therapies. The potential of SSR targeted therapies is currently limited by the fact that the receptor is not uniquely specific for cancer cells.

Somatostatin receptors are present in important normal tissues including the brain, pituitary, adrenal glands, pancreas, gastrointestinal tract, and kidney. The following references relate to this subject matter: Forssell-Aronsson E. B., et al.,” 111In-DTPA-D-Phel-octreotide Binding and Somatostatin Receptor Subtypes in Thyroid Tumors,” J Nucl Med, 41 (4):636-42 (2000); Sulkowski U., et al., “A Phase II Study of High-Dose Octreotide in Patients with Unresectable Pancreatic Carcinoma,” Eur J Cancer, 35(13):1805-8 (1999); Reubi J. C., et al., “A Selective Analog for the Somatostatin sstl -Receptor Subtype Expressed by Human Tumors,” Eur J Pharmacol, 345(1):103-10 (1998); O'Byrne K. J. et al., “Phase II Study of RC-160 (Vapreotide), an Octapeptide Analogue of Somatostatin, in the Treatment of Metastatic Breast Cancer,” Br J Cancer, 79(9-10):1413-8 (1999); Bajc M., et al., “Dynamic Indium-111-Pentetreotide Scintigraphy in Breast Cancer,” J Nucl Med, 37(4):622-6 (1996); Schally A. V.; Nagy A., “Cancer Chemotherapy Based on Targeting of Cytotoxic Peptide Conjugates to their Receptors on Tumors,” Eur J Endocrinol, 141(1):1-14 (1999Nagy A., et al., “Synthesis and Biological Evaluation of Cytotoxic Analogs of Somatostatin Containing Doxorubicin or its Intensely Potent Derivative, 2-Pyrrolinodoxorubicin,” Proc Natl Acad Sci USA, 95(4):1794-9 (1998); Sinisi A. A., et al., “Different Expression Patterns of Somatostatin Receptor Subtypes in Cultured Epithelial Cells from Human Normal Prostate and Prostate Cancer,” J Clin Endocrinol Metab, 82(8):2566-9 (1997); Albert R., et al., “Direct Synthesis of [DOTA-DPhe1]-octreotide and [DOTA-DPhel,Tyr3]-octreotide (SMT487): Two Conjugates for Systemic Delivery of Radiotherapeutical Nuclides to Somatostatin Receptor Positive Tumors in Man,.” Bioorg Med Chem Lett, 8(10):1207-10 (1998); Reubi Plonowski A., et al., “Inhibition of PC-3 Human Androgen-Independent Prostate Cancer and its Metastases by Cytotoxic Somatostatin Analogue AN-238,” Cancer Res, 59(8):1947-53 (1999); Papotti M., et al., “Correlative Immunohistochemical and Reverse Transcriptase Polymerase Chain Reaction Analysis of Somatostatin Receptor Type 2 in Neuroendocrine Tumors of the Lung,” Diagn Mol Pathol, 9(1):47-57 (2000); Moertel C. L., et al., “Expression of Somatostatin Receptors in Childhood Neuroblastoma,” Am J Clin Pathol, 102(6):752-6 (1994); Lewis J. S., et al., “In vitro and in Vivo Evaluation of 64Cu-TETA-Tyr3-Octreotate. a New Somatostatin Analog with Improved Target Tissue Uptake,” Nucl Med Biol, 26(3):267-73 (1999); Plonowski A., et al., “Inhibition of PC-3 Human Androgen-Independent Prostate Cancer and its Metastases by Cytotoxic Somatostatin Analogue AN-238,” Cancer Res, 59(8):1947-53 (1999); Koppan M., et al., “Targeted Cytotoxic Analogue of Somatostatin AN-238 Inhibits Growth of Androgen-Independent Dunning R-3327-AT-1 Prostate Cancer in Rats at Nontoxic Doses,” Cancer Res, 58(18):4132-7 (1998); Nilsson S., et al., “Metastatic Hormone-Refractory Prostatic Adenocarcinoma Expresses Somatostatin Receptors and is Visualized in Vivo by [111In]-labeled DTPA-D-[Phe1]-octreotide Scintigraphy,” Cancer Res, 55(23 Suppl):5805s-5810s (1995); Briganti V., et al., “Imaging of Somatostatin Receptors by Indium-11-Pentetreotide Correlates with Quantitative Determination of Somatostatin Receptor Type 2 Gene Expression in Neuroblastoma Tumors,” Clin Cancer Res, 3(12 Pt 1):2385-91 (1997); Thakur M. L., et al., “Radiolabeled Somatostatin Analogs in Prostate Cancer,” Nucl Med Biol, 24(1):105-13 (1997); Reubi J. C.; Kvols L., “Somatostatin Receptors in Human Renal Cell Carcinomas,” Cancer Res, 52(21):6074-8 (1992); Vitale G., et al., “Slow Release Lanreotide in Combination with Interferon-Alpha2b in the Treatment of Symptomatic Advanced Medullary Thyroid Carcinoma,” J Clin Endocrinol Metab, 85(3):983-8 (2000); Reisinger I., et al., “Somatostatin Receptor Scintigraphy in Small-Cell Lung Cancer: Results of a Multicenter Study,” J Nuci Med, 39(2):224-7 (1998); Albini A., et al., “Somatostatin Controls Kaposi's Sarcoma Tumor Growth through Inhibition of Angiogenesis,” FASEB J, 13:647-655 (1999); Reubi J. C., et al., “Expression and Localization of Somatostatin Receptor SSTR1, SSTR2, and SSTR3 Messenger RNas in Primary Human Tumors using in Situ Hybridization,” Cancer Res, 54(13):3455-9 (1994); Vuaroqueaux V., et al., “No Loss of sst Receptors Gene Expression in Advanced Stages of Colorectal Cancer,” Eur J Endocrinol, 140(4):362-6 (1999); Kahan Z., et al., “Inhibition of Growth of MX-1, MCF-7-MIII and MDA-MB-231 Human Breast Cancer Xenografts after Administration of a Targeted Cytotoxic Analog of Somatostatin, AN-238,” Int J Cancer, 82(4):592-8 (1999); Krenning E. P., et al., “The Role of Radioactive Somatostatin and its Analogues in the Control of Tumor Growth,” Recent Results Cancer Res, 153:1-13 (2000); Kath R.; Hoffken K. “The “Yttrium-90 DOTATOC: First Clinical Results,” Eur J Nucl Med, 26(11):1439-47 (1999); Friedberg et al., “Octreotide Scanning in Metastatic Sarcoma,” Cancer, 86:1621-7 (1999); Froidevaux S., et al., “Receptor Targeting for Tumor Localisation and Therapy with Radiopeptides,” Curr Med Chem, 7(9):971-994 (2000); Freidinger R. M., “Nonpeptidic Ligands for Peptide and Protein Receptors,” Curr Opin Chem Biol, 3(4):395-406 (1999), the contents of which are incorporated herein by reference in their entirety.

A preferred embodiment of the present invention is a compound ET with a targeting ligand comprised of a structure that binds to somatostatin receptors. A preferred embodiment is based on the ability of a class of small peptidomimetics that bind to type 2 SSR with picomolar potency. The following references relate to this subject matter: Yang L., et al., “Synthesis and Biological Activities of Potent Peptidomimetics Selective for Somatostatin Receptor Subtype 2,” Proc Natl Acad Sci USA, 95(18):10836-41 (1998), the contents of which are incorporated herein by reference in their entirety.

In preferred embodiments (TL32 and TL33) the targeting ligands comprise the following structures:

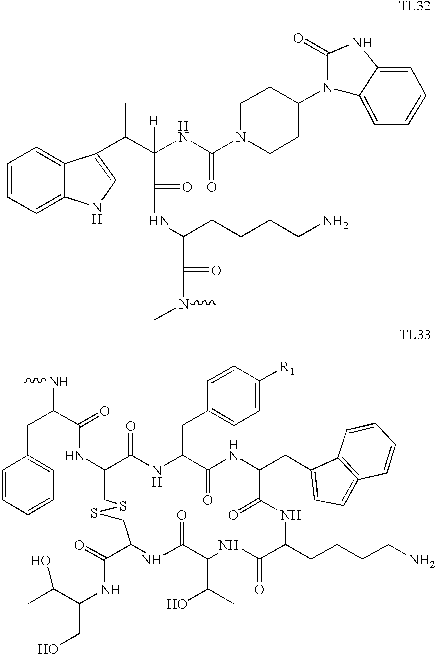

wherein the wavy line is the site of linker attachment to the remainder of the drug complex.

Other preferred embodiments are based on the ability of somatostatin analogs substituted on the amino terminus with chelating agents to retain the ability to bind to SSR. The following references relate to this subject matter: Lewis J. S., et al., “Comparison of Four ⁶⁴Cu-Labeled Somatostatin Analogues in Vitro and in a Tumor-Bearing Rat Model: Evaluation of New Derivatives for Positron Emission Tomography Imaging and Targeted Radiotherapy,” J Med Chem, 42(8):1341-1347 (1999), the contents of which are incorporated herein by reference in their entirety.

Wherein for TL33 the wavy line is the site of linker attachment to the remainder of the drug complex and R₁ is H, or OH, and the terminal phenylalanine and the tryptophan have the D- configuration and the remainder of the amino acid residues have the L-configuration.

Gastrin Releasing Peptide Receptor Targeting Ligands

Gastrin releasing peptide receptors (GRPR) are over-expressed in a variety of malignancies including: lung, breast, prostate, colorectal, gastric, and melanoma. Gastrin releasing peptide is produced in small cell lung carcinoma in an autocrine manner and stimulates cell growth by binding to GRPR. A large variety of radiolabelled GRPR analogs have been developed. Conjugates of potent cytotoxic agents have been coupled to GRPR binding groups as potential antineoplastic drugs. In addition a large number of analogs that bind to GRPR have been investigated as anti-cancer therapies. However, GRPR is not specific to malignant cells, which currently limits its utility as an anti-cancer target.

Normal tissues that express significant amounts of GRPR include the gastric antrum, breast, ovarian, pancreas, brain, and skin. The following references relate to this subject mafter: Karra S. R., et al., “^{99m}Tc-Labeling and in Vivo Studies of a Bombesin Analogue with a Novel Water-Soluble Dithiadiphosphine-Based Bifunctional Chelating Agent,” Bioconjugate Chem, 10(2):254-260 (1999); Carroll R. E., et al., “Aberrant Expression of Gastrin-Releasing Peptide and its Receptor by Well-Differentiated Colon Cancers in Humans,” AJP-Gastrointestinal and Liver Physiology, 276 (3):G655-G665 (1999); Wang Q. J., et al., “Bombesin Can Stimulate Proliferation of Human Pancreatic Cancer Cells through an Autocrine Pathway,” Int J Cancer, 68(4):528-34 (1996); Carroll R. E., et al., “Characterization of Gastrin-Releasing Peptide Receptors Aberrantly Expressed by Non-Antral Gastric Adenocarcinomas,” Peptides, 20(2):229-37 (1999); Chave H. S., et al., “Bombesin Family Receptor and Ligand Gene Expression in Human Colorectal Cancer and Normal Mucosa,” Br J Cancer, 82(1):124-30 (2000); Azay J., et al., “Comparative Study of in Vitro and in Vivo Activities of Bombesin Pseudopeptide Analogs Modified on the C-Terminal Dipeptide Fragment,” Peptides, 19(1):57-63 (1998); Nagy A., et al., “Design, Synthesis, and in Vitro Evaluation of Cytotoxic Analogs of Bombesin-Like Peptides Containing Doxorubicin or its Intensely Potent Derivative, 2-Pyrrolinodoxorubicin,” Proc Natl Acad Sci U S A, 94(2):652-6 (1997); Baidoo K. E., et al., “Design, Synthesis, and Initial Evaluation of High-Affinity Technetium Bombesin Analogues,” Bioconjugate Chem., 9(2):218-225, (1998); Breeman W. A., et al., “Evaluation of Radiolabelled Bombesin Analogues for Receptor-Targeted Scintigraphy and Radiotherapy,” Int J Cancer, 81(4):658-65 (1999); Tang C., et al. “Expression of Receptors for Gut Peptides in Human Pancreatic Adenocarcinoma and Tumour-Free Pancreas,” Br J Cancer, 75(10):1467-73 (1997); Sainz E., et al., “Four Amino Acid Residues are Critical for High Affinity Binding of Neuromedin B to the Neuromedin B Receptor,” J Biol Chem, 273(26):15927-15932 (1998); Kiaris H., et al., “Targeted Cytotoxic Analogue of Bombesin/Gastrin-Releasing Peptide Inhibits the Growth of H-69 Human Small-Cell Lung Carcinoma in Nude Mice,” Br J Cancer, 81(6):966-71 (1999); Gugger M.; Reubi J. C., “Gastrin-Releasing Peptide Receptors in Non-Neoplastic and Neoplastic Human Breast,” American Journal of Pathology, 155:2067-2076 (1999); Markwalder R.; Reubi J. C., “Gastrin-Releasing Peptide Receptors in the Human Prostate: Relation to Neoplastic Transformation,” Cancer Res, 59(5):1152-9 (1999); Sun B., et al., “The Presence of Receptors for Bombesin/GRP and Mrna for Three Receptor Subtypes in Human Ovarian Epithelial Cancers,” Regul Pept, 90(1-3):77-84 (2000); Sun B., et al., “Presence of Receptors for Bombesin/Gastrin-Releasing Peptide and Mrna for Three Receptor Subtypes in Human Prostate Cancers,” Prostate, 42(4):295-303 (2000); Pradhan T. K., et al. “Identification of a Unique Ligand which has High Affinity for all Four Bombesin Receptor Subtypes,” Eur J Pharmacol, 343(2-3):275-87 (1998); Pansky A., et al., “Identification of Functional GRP-Preferring Bombesin Receptors on Human Melanoma Cells,” Eur J Clin Invest, 27(1):69-76 (1997); Bartholdi M. F., et al., “In Situ Hybridization for Gastrin-Releasing Peptide Receptor (GRP Receptor) Expression in Prostatic Carcinoma,” Int J Cancer, 79(1):82-90 (1998); Jungwirth A., et al.,” Inhibition of Growth of Androgen-Independent DU-145 Prostate Cancer in Vivo by Luteinising Hormone-Releasing Hormone Antagonist Cetrorelix and Bombesin Antagonists RC-3940-II and RC-3950-II,” Eur J Cancer, 33(7):1141-8 (1997); Kahan Z., et al., “Inhibition of Growth of MDA-MB-468 Estrogen-Independent Human Breast Carcinoma by Bombesin/Gastrin-Releasing Peptide Antagonists RC-3095 and RC-3940-II,” Cancer, 88(6):1384-92 (2000); Ferris H. A., et al., “Location and Characterization of the Human GRP Receptor Expressed by Gastrointestinal Epithelial Cells,” Peptides, 18(5):663-72 (1997); Toi-Scott M., et al., “Clinical Correlates of Bombesin-Like Peptide Receptor Subtype Expression in Human Lung Cancer Cells,” Lung Cancer, 15(3):341-54 (1996); Jungwirth A., et al., “Luteinizing Hormone-Releasing Hormone Antagonist Cetrorelix (SB-75) and Bombesin Antagonist RC-3940-II Inhibit the Growth of Androgen-independent PC-3 Prostate Cancer in Nude Mice,” Prostate, 32(3):164-72 (1997); Safavy A., et al., “Paclitaxel Derivatives for Targeted Therapy of Cancer: Toward the Development of Smart Taxanes,” J. Med. Chem., 42(23):4919-4924 (1999); Katsuno T., et al., “Pharmacology and Cell Biology of the Bombesin Receptor Subtype 4 (BB₄-R),” Biochemistry, 38(22):7307-7320 (1999); Breeman W. A., et al., “Pre-clinical Evaluation of [(111)In-DTPA-Pro(1), Tyr(4)]Bombesin, a New Radioligand for Bombesin-Receptor Scintigraphy,” Int J Cancer, 83(5):657-63 (1999); Halmos G.; Schally A. V., “Reduction in Receptors for Bombesin and Epidermal Growth Factor in Xenografts of Human Small-Cell Lung Cancer after Treatment with Bombesin Antagonist RC-3095,” Proc. Nati. Acad. Sci. USA, 94:956-960 (1997); Miyazaki M., et al., “Inhibition of Growth of MDA-MB-231 Human Breast Cancer Xenografts in Nude Mice by Bombesin/Gastrin-Releasing Peptide (GRP) Antagonists RC-3940-II and RC-3095,” Eur J Cancer, 34(5):710-7 (1998); Staniek V., et al., “Expression of Gastrin-Releasing Peptide Receptor in Human Skin,” Acta Derm Venereol, 76(4):282-6 (1996); Llinares M., et al., “Syntheses and Biological Activities of Potent Bombesin Receptor Antagonists,” J Pept Res, 53(3):275-83 (1999); Cristau M., et al., “Synthesis and Biological Evaluation of Bombesin Constrained Analogues,” Med Chem ASAP Article, 10:1021 (Can 12, 2000); Carrithers M. D.; Lerner M. R., “Synthesis and Characterization of Bivalent Peptide Ligands Targeted to G-Protein-Coupled Receptors,” Chem Biol, 3(7):537-42 (1996); Safavy A., et al., “Synthesis of Bombesin Analogues for Radiolabeling with Rhenium-188,” Cancer, 80(12 Suppl):2354-9 (1997); Slice L. W., et al., “Visualization of Internalization and Recycling of the Gastrin Releasing Peptide Receptor-Green Fluorescent Protein Chimera Expressed in Epithelial Cells,” Receptors Channels, 6(3):201-12 (1998), the contents of which are incorporated herein by reference in their entirety.

A preferred embodiment of the present invention is a compound ET with a targeting ligand comprised of a structure that binds to GRPR. A preferred embodiment is based upon the ability of a bombesin analog with a chelating agent coupled to the amino terminus to bind with high affinity to GRPR. The following references relate to this subject matter: Karra S. R., et al., “^{99m}Tc-Labeling and in Vivo Studies of a Bombesin Analogue with a Novel Water-Soluble Dithiadiphosphine-Based Bifunctional Chelating Agent,” Bioconjugate Chem, 10(2):254-260 (1999), the contents of which are incorporated herein by reference in their entirety.

In preferred embodiments (TL34 and TL35) the targeting ligands comprise the following structures:

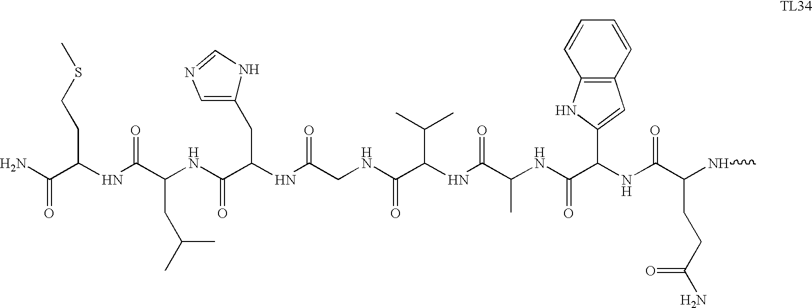

wherein the wavy line is the site of linker attachment to the remainder of the drug.

Another preferred embodiment is based on the ability of the nonpeptide gastrin releasing receptor antagonist to bind with high affinity to GRPR.

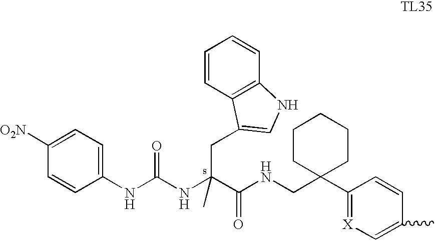

wherein the wavy line is the site of linker attachment to the remainder of the drug, and X is C, or N. The following references relate to this subject matter: Ashwood V., et al., “PD 176252—The First High Affinity Non-Peptide Gastrin-Releasing Peptide (BB2) Receptor Antagonist,” Bioorg Med Chem Lett, 8(18):2589-94 (1998), the contents of which are incorporated herein by reference in their entirety.

Melanocyte Stimulating Hormone Receptor Targeting Ligands

Melanocyte Stimulating Hormone Receptors (MSHR) bind melanocyte stimulating hormone and related peptide factors with high affinity. The MSH receptors are localized to melanocytes, keratinocytes, monocytes, macrophages, and the dermal microvasculature. The consistent expression of MSHR in malignant melanoma has stimulated efforts to employ the receptor for diagnostic imaging and chemotherapy targeting. A large number of potent analogs are known which bind with high affinity to this receptor. Compounds with multiple copies of MSHR binding ligands have been prepared for diagnostic and potential therapeutic use. MSHR are present at low density with approximately 2,000 to 10,000 receptor molecules/cell. The low receptor density necessitates the delivery of an extremely potent toxin that is cytotoxic at sub-nanomolar concentrations to kill the MSHR positive tumor cells. To achieve selective toxicity the concentration of targeted drug required to saturate the receptors on the tumor cells must be even lower. Also, melanoma cells secrete melanocyte stimulating hormone, which can act as a competitive inhibitor to MSHR targeted drugs. Accordingly, targeting affinity in the sub-picomolar range is required, which markedly exceeds the high binding affinity of currently known MSHR ligands. Even given a ligand with the requisite affinity the problem of toxicity to non-tumor MSHR positive keratinocytes and dermal blood vessels remains. Both of these problems can be solved with the present invention by utilizing a multifunctional drug delivery vehicle that incorporates a MSHR binding ligand a second ligand that binds with high affinity to a second target present at high concentrations on melanoma cells, but absent or present at low levels on keratinocytes and derrmal microvasculature. The following references relate to this subject matter: Tsatmali M., et al., “ACTH1-17 is a More Potent Agonist at the Human MC1 Receptor than Alpha-MSH,” Cell Mol Biol (Noisy-le-grand), 45(7):1029-34 (1999); Hruby V. J., et al., “Cyclic Lactam Alpha-Melanotropin Analogues of Ac-Nle4-cyclo[Asp5, D-Phe7,Lys10] Alpha-Melanocyte-Stimulating Hormone-(4-10)-NH2 with Bulky Aromatic Amino Acids at Position 7 Show High Antagonist Potency and Selectivity at Specific Melanocortin Receptors,” J Med Chem, 38(18):3454-61 (1995); Funasaka Y., et al., ‘Expression of Proopiomelanocortin, Corticotropin-Releasing Hormone (CRH), and CRH Receptor in Melanoma Cells, Nevus Cells, and Normal Human Melanocytes,” J Investig Dermatol Symp Proc, 4(2):105-9 (1999); Vaidyanathan G.; Zalutsky M. R., “Fluorine-18-labeled [Nle4,D-Phe7]-alpha-MSH, an Alpha-Melanocyte Stimulating Hormone Analogue,” Nucl Med Biol, 24(2):171-8 (1997); Jiang J., et al., “Human Epidermal Melanocyte and Keratinocyte Melanotropin Receptors: Visualization by Melanotropic Peptide Conjugated Macrospheres (Polyamide Beads),” Exp Dermatol, 6(1):6-12 (1997); Hartmeyer M., et al., “Human Dermal Microvascular Endothelial Cells Express the Melanocortin Receptor Type 1 and Produce Increased Levels of IL-8 Upon Stimulation with Alpha-Melanocyte-Stimulating Hormone,” J Immunol, 159(4):1930-7 (1997); Loir B., et al., “Immunoreactive Alpha-Melanotropin as an Autocrine Effector in Human Melanoma Cells,” Eur J Biochem, 244(3):923-30 (1997); Loir B., et al., “Expression of the MC1 Receptor Gene in Normal and Malignant Human Melanocytes. A Semiquantitative RT-PCR Study,” Cell Mol Biol (Noisy-le-grand), 45(7):1083-92 (1999); Rajora N., et al., “Alpha-MSH Production, Receptors, and Influence on Neopterin in a Human Monocyte/Macrophage Cell Line,” J Leukoc biol, 59(2):248-53 (1996); Sharma S. D., et al., “Melanotropic Peptide-Conjugated Beads for Microscopic Visualization and Characterization of Melanoma Melanotropin Receptors,” Proc Natl Acad Sci USA, 93:13715-13720 (1996); Jiang J., et al., “Melanotropic Peptide Receptors: Membrane Markers of Human Melanoma Cells,” Exp Dermatol, 5(6):325-33 (1996); Ghanem G. E., et al., “Human Melanoma Targeting with Alpha-MSH-Melphalan Conjugate,” Melanoma Res, 1(2):105-14 (1991); Hadley M. E., et al., “[Nle4, D-Phe7]-alpha-MSH: A Superpotent Melanotropin that “Irreversibly” Activates Melanoma Tyrosinase,” Endocr Res, 11(3-4):157-70 (1985); Sharma S. D. et al., “Multivalent Melanotropic Peptide and Fluorescent Macromolecular Conjugates: New Reagents for Characterization of Melanotropin Receptors,” Bioconjug Chem, 5(6):591-601 (1994); Giblin M. F., et al., “Design and Characterization of α-Melanotropin Peptide Analogs Cyclized through Rhenium and Technetium Metal Coordination,” PNAS Online, 95(22):12814-12818 (1998); O'Hare K. B, et al., “Polymeric Drug-Carriers Containing Doxorubicin and Melanocyte-Stimulating Hormone: In Vitro and in Vivo Evaluation against Murine Melanoma,” J Drug Target, 1(3):217-29 (1993); Bagufti C., et al., “[111In]-DTPA-labeled Analogues of Alpha-Melanocyte-Stimulating Hormone for Melanoma Targeting: Receptor Binding in Vitro and in Vivo,” Int J Cancer, 58(5):749-55 (1994); Morandini R., et al., “Receptor-Mediated Cytotoxicity of Alpha-MSH Fragments Containing Melphalan in a Human Melanoma Cell Line,” Int J Cancer, 56(1):129-33 (1994); Ghanem G. E., et al., “Evidence for alpha-Melanocyte-Stimulating Hormone (Alpha-MSH) Receptors on Human Malignant Melanoma Cells,” Int J Cancer, 41(2):248-55 (1988); Bednarek M. A., et al., “Structure-Function Studies on the Cyclic Peptide MT-II, Lactam Derivative of Alpha-Melanotropin,” Peptides, 20(3):401-9 (1999); Chaturvedi D. N., et al., “Synthesis and Biological Evaluation of the Superagonist [N Alpha-Chlorotriazinylaminofluorescein-Ser1, Nle4,D-Phe7]-apha-MSH,” J Pharm Sci, 74(3):237-40 (1985); Giblin M. F., et al., “Synthesis and Characterization of Rhenium-Complexed -Melanotropin Analogs,” Bioconjugate Chem, 8(3):347-353 (1997); Brandenburger Y., et al., “Synthesis and Receptor Binding Analysis of Thirteen Oligomeric Alpha-MSH Analogs,” J Recept Signal Transduct Res, 19(1-4):467-80 (1999); Erskine-Grout M. E, et al., “Melanocortin Probes for the Melanoma MC1 Receptor: Synthesis, Receptor Binding and Biological Activity,” Melanoma Res, 6(2):89-94 (1996); Haskell-Luevano C., et al., “Truncation Studies of Alpha-Melanotropin Peptides Identify Tripeptide Analogues Exhibiting Prolonged Agonist Bioactivity,” Peptides, 17(6):995-1002 (1996), the contents of which are incorporated herein by reference in their entirety.

A preferred embodiment of the present invention is a compound ET with a targeting ligand comprised of a structure that binds to MSHR. Preferred embodiments are based on some melanotropin analogs, which possess extremely high receptor affinity. It is known that the amino terminus can be substituted without impairing receptor binding. The following references relate to this subject matter: Haskell-Luevano C., et al., “Characterizations of the Unusual Dissociation Properties of Melanotropin Peptides from the Melanocortin Receptor, hMC1 R,” J Med Chem, 39:432-435 (1996), the contents of which are incorporated herein by reference in their entirety.

In preferred embodiments (TL36 and TL37) the targeting ligands comprise the following structures:

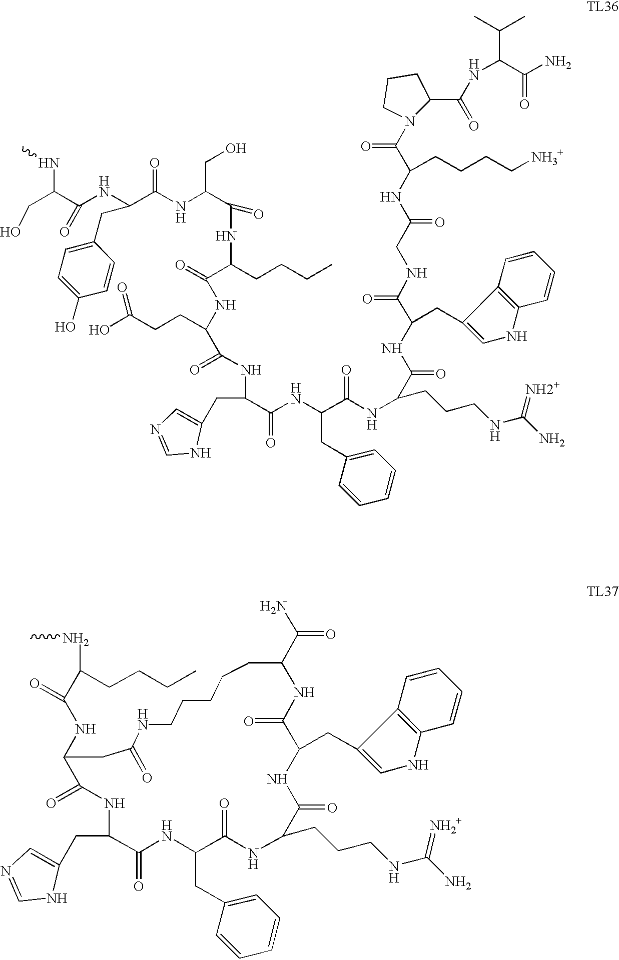

wherein the wavy line is the site of linker attachement to the remainder of the drug complex. The following references relate to this subject matter: Haskell-Luevano C., et al., “Biological and Conformational Examination of Stereochemical Modifications Using the Template Melanotropin Peptide, Ac-NIe-c[Asp-His-Phe-Arg-Trp-Ala-Lys]-NH₂, on Human Melanocortin Receptors,” J Med Chem, 40:1738-1748 (1997), the contents of which are incorporated herein by reference in their entirety.

Gastrin/Cholecystokinin Type B Receptor Targeting Ligands

Gastrin/Cholecystokinin Type B Receptor (CCKBR) are enriched on the membranes of a variety of human malignancies including: medullary thyroid cancer, small cell lung cancer, astrocytomas, stromal ovarian cancers, and occasionally in gastroenteropancreatic tumors, breast, endometrial, and ovarian adenocarcinomas. CCKBR are present normally in the stomach, and brain. CCKBR selective ligands coupled to cytotoxin and radionuclides have been described as potential tumor therapeutic and diagnostic agents. However, the potential is severely limited by the expression of CCKBR by normal important tissues. The following references relate to this subject matter: Czerwinski G., et al., “Cytotoxic Agents Directed to Peptide Hormone Receptors: Defining the Requirements for a Successful Drug,” Proc Nati Acad Sci USA, 95(20):11520-5 (1998); de Jong M., et al., “Preclinical and Initial Clinical Evaluation of 111In-Labeled Nonsulfated CCK8 Analog: A Peptide for CCK-B Receptor-Targeted Scintigraphy and Radionuclide Therapy,” J Nucl Med, 40(12):2081-7 (1999); Behr T. M., et al., “Targeting of Cholecystokinin-B/Gastrin Receptors in Vivo: Preclinical and Initial Clinical Evaluation of the Diagnostic and Therapeutic Potential of Radiolabelled Gastrin,” Eur J Nucl Med, 25(4):424-30 (1998); Sinha J., et al., “Quantitative Structure-Activity Relationship Study on Some Nonpeptidal Cholecystokinin Antagonists,” Bioorg Med Chem, 7(6):1127-30 (1999); Behr T. M., et al., “Radiolabeled Peptides for Targeting Cholecystokinin-B/Gastrin Receptor-Expressing Tumors,” J Nucl Med, 40(6):1029-44 (1999); Biagini P., et al., “The human Gastrin/Cholecystokinin Receptors: Type B and Type C Expression in Colonic Tumors and Cell Lines,” Life Sci, 61(10):1009-18 (1997); Reubi J. C.; Waser B., “Unexpected High Incidence of Cholecystokinin-B/Gastrin Receptors in Human Medullary Thyroid Carcinomas,” Int J Cancer, 67(5):644-7 (1996); Reubi J. C., et al., “Unsulfated DTPA- and DOTA-CCK Analogs as Specific High-Affinity Ligands for CCK-B Receptor-Expressing Human and Rat Tissues in Vitro and in Vivo,” Eur J Nucl Med, 25(5):481-90 (1998); the contents of which are incorporated herein by reference in their entirety.

A preferred embodiment of the present invention is a compound ET with a targeting ligand comprised of a structure that binds to CCKBR.

A large number of groups, which bind to CCKBR with high affinity, are known. A preferred embodiment shown below is based upon the high affinity binding of certain benzodiazepam analog for the CCKBR. The following references relate to this subject matter: Showell G. A., et al., “High-Affinity and Potent, Water-Soluble 5-Amino-1,4-Benzodiazepine CCKB/Gastrin Receptor Antagonists Containing a Cationic Solubilizing Group,” J Med Chem, 37(6):719-21 (1994), the contents of which are incorporated herein by reference in their entirety.

In preferred embodiments (TL38 , TL39, andTL40) the targeting ligands comprise the following structures:

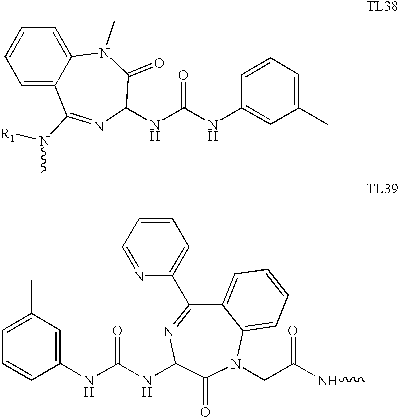

wherein the wavy line is the site of linker attachment, and R₁ is H, or methyl, or ethyl.

The following reference relates to this subject matter: Matassa V. G., “5-(Piperidin-2-yl)- and 5-(Homopiperidin-2-yl)-1,4-benzodiazepines: High-Affinity, Basic Ligands for the Cholecystokinin-B Receptor,” J Med Chem, 40(16):2491-2501 (1997), the contents of which is incorporated herein by reference in its entirety.

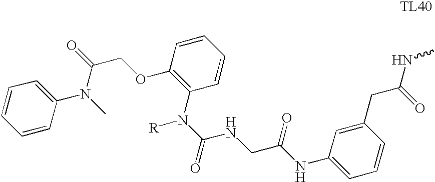

wherein the wavy line is the site of linker attachment and R is benzyl, cyclohexylmethyl, cyclohexylethyl, cyclohexylpropyl, cyclopropylmethyl, cyclobutylmethyl, cyclopentylmethyl, cycloheptylmethyl, 2-methylpropyl, 2,2,dimethylpropyl, 3-methylbutyl, n-butyl, 2-ethylbutyl, 3-methylpentyl, 4-methyl-3-pentenyl, or 4-methylpentyl.

The following reference relates to this subject matter: Takeda Y., et al., “Synthesis of Phenoxyacetic Acid Derivatives as Highly Potent Antagonists of Gastrin/Cholecystokinin-B Receptors. III,” Chem Pharm Bull (Tokyo), 47(6):755-71 (1999), the contents of which is incorporated herein by reference in its entirety.

Guanidinobenzoatase Selective Targeting Ligands

Guanidinobenzoatase GB is protease that is enriched on the surface of most human malignancies. There is evidence that GB is a precursor to a tumor-associated collagenase. Selective stains for GB have been employed to identify malignant cells in pathology specimens. GB binds to a variety of guanidino and amino analogs. GB Selective ligands coupled to mitomycin C and adriamycin have been described as potential anti-cancer agents. However, GB is not specific to malignant cells and as a solo targeting factor is unlikely to provide sufficient tumor selectivity. GB is also present on the surface of normal colonic epithelial cells. The following references relate to this subject matter: Steven F., et al., “A Fluorescent Study of Ligands for Guanidinobenzoatase, a Protease Associated with Tumour Cells,” Anti-cancer Res, 8(6):1179-83 (1988); Steven F. S., et all, “A Simple Fluorescent Technique for the Location of Tumour Cells in Frozen Sections of the Head and Neck Region,” Anti-cancer Res, 11(3):1189-94 (1991); Poustis-Delpont C., et al., “Characterization and Purification of a Guanidinobenzoatase: A Possible Marker of Human Renal Carcinoma,” Cancer Res, 52(13):3622-8 (1992); Anees M.; Benbow E. W. et al., “Dansyl Fluoride, a Fluorescent Inhibitor for the Location of Tumour Cells in Human Tissues,” J Enzyme Inhib, 10(3):195-201 (1996); Thaon S., et al., “Differential SP220K Expression in Renal Carcinoma and Oncocytoma Cells,” Int J Cancer, 72(5):752-7 (19973); Steven F. S., et al., “Fluorescent Location of Malignant Cells in Smears Obtained From Sputum,” Anti-cancer Res, 14(5A):2021-4 (1994); Steven F. S., et al., “Fluorescent Location of Abnormal Cells in Cell Smears Obtained from the Lungs of Patients with Lung Cancer,” Anti-cancer Res, 12(3):625-9 (1992); Steven F. S., et al., “Fluorescent Locaton of Tumour Cells in Fine Needle Aspirates,” Anti-cancer Res, 11(5):1697-9 (1991); Bernstein L. J., et al., “Guanidinobenzoatase and UPA in High-Grade Human Astrocytomas and after Xenografting Cell Suspensions into the Rat Cerebral Cortex: Proteases for Metastasis and Disease Progression,” Anti-cancer Res, 18(4A):2583-90 (1998); Steven F. S., et al., “GB (Guanidinobenzoatase) Cell Surface Protease and Serum Inhibitors in Colorectal Neoplasia,” J Pathol, 167(1):19-24 (1992); Anees M.; Steven F. S., et al., “Inhibition of a Tumour Protease with 3,4-Dichloroisocoumarin, Pentamidine-Isethionate and Guanidino Derivatives,” J Enzyme Inhib, 8(3):213-21 (1994); Anees M., “Interaction of Tissue Plasminogen Activator Inhibitor with Cell Surface Guanidinobenzoatase and Urokinase Plasminogen Activator,” J Enzyme Inhib, 10(4):281-8 (1996); Steven F. S., et al., “Labelling of Tumour Cells with a Biotinylated Inhibitor of a Cell Surface Protease,” J Enzyme Inhib, 4(4):337-46 (1991); Anees M., “Location of Tumour Cells in Colon Tissue by Texas Red Labelled Pentosan Polysulphate, an Inhibitor of a Cell Surface Protease,” J Enzyme Inhib, 10(3):203-14 (1996); Steven F. S., et al., “Studies on the Activity of a Protease Associated with Cells at the Advancing Edge of Human Tumour Masses in Frozen Sections,” Br J Cancer, 58(1):57-60 (1988); Steven F. S., et al., “Targeting Adriamycin to Tumour Cells by Means of an Affinity Ligand; A Model System for Drug Delivery,” Anti-cancer Res, 9(1):247-53 (1989); Steven F. S., et al., “The Targeting of Agmatine-Liganded Mitomycin C to an Enzyme on the Surface of Tumour Cells,” Anti-cancer Res, 10(3):583-9 (1990); Steven F. S., et al., “The Design of Fluorescent Probes which Bind to the Active Centre of Guanidinobenzoatase. Application to the Location of Cells Possessing this Enzyme,” Eur J Biochem, 149(1):35-40 (1985); Poustis-Delpont C., et al., “Monomeric 55-kDa Guanidinobenzoatase Switches to a Serine Proteinase Activity upon Tetramerization. Tetrameric Proteinase SP 220 K Appears as the Native Form,” J Biol Chem, 269(20):14666-71 (1994), the contents of which are incorporated herein by reference in their entirety.

A preferred embodiment of the present invention is a compound ET with a targeting ligand comprised of a structure that binds to GB.

In preferred embodiment (TL41) the targeting ligand comprises the following structure:

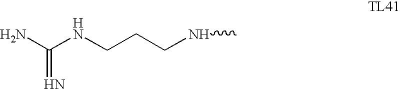

wherein the wavy line is the site of linker attachment.

Norepinephrine Transporter Selective Ligands

A variety of neuroendocrine malignancies including neuroblastoma, and malignant pheochromocytomas have increased expression of the norepinephrine transporter (NET). M-lodobenzylguanidine, which binds to NET, has been utilized for diagnosis and therapy of NET+ malignancies. Fluorescent analogs have also been described as potential diagnostic aids. The following references relate to this subject matter: Nakagami Y., et al., “A Case of Malignant Pheochromocytoma Treated with 131I-Metaiodobenzylguanidine and Alpha-Methyl-P-Tyrosine,” Jpn J Med, 29(3):329-33 (1990); Smets L. A., et al., “Extragranular Storage of the Neuron Blocking Agent Meta-lodobenzylguanidine (MIBG) in Human Neuroblastoma Cells,” Biochem Pharmacol, 39(12):1959-64 (1990); Gelfand M. J., et al., “Meta-lodobenzylguanidine in Children,” Semin Nucl Med, 23(3):231-42 (1993); Hadrich D., et al., “Synthesis and Characterization of Fluorescent Ligands for the Norepinephrine Transporter: Potential Neuroblastoma Imaging Agents,” J Med Chem, 42(16):3101-8 (1999); Beierwaltes W. H., et al., “Update on Basic Research and Clinical Experience with Metaiodobenzylguanidine,” Med Pediatr Oncol, 15(4):163-9 (1987), the contents of which are incorporated herein by reference in their entirety.

A preferred embodiment of the present invention is a compound ET with a targeting ligand comprised of a structure that binds to the norepinephrine transporter.

In preferred embodiments (TL42 , TL43, and TL44) the targeting ligands comprise the following structures:

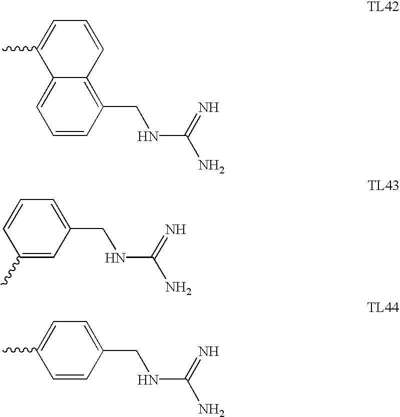

wherein the wavy line is the site of linker attachment.

Monoclonal Antibody Based Multifunctional Drug Delivery Vehicles

Monoclonal antibody-toxin conjugates are well known. It has been 25 years since the landmark development of monoclonal antibodies by Kohler and Milstein. The following reference relates to this subject matter: Kohler G.; Milstein C., “Continuous Cultures of Fused Cells Secreting Antibody of Predefined Specificity,” Nature, 256:495-497 (1975), the contents of which is incorporated herein by reference in its entirety.

Despite enormous efforts, only a handful of monoclonal antibody based drugs have been approved for clinical use. Monoclonal antibodies offer the promise of exquisite targeting specificity. However, there are actually very few antigenic targets known which are absolutely specific for malignant cells. Attempts to utilize anti-Lewis Y monoclonal antibodies conjugated to the anti-cancer drug doxorubicin illustrate the problem. The Lewis Y antigen is an excellent tumor-associated antigen, which is enriched on the majority of epitheal malignancies including: breast cancer, colon cancer, non small cell lung cancer, cervical cancer, ovarian cancer and melanoma. The conjugate known as Br96-Doxorubicin, when evaluated in women with metastatic breast cancer, was found to be less effective than non-targeted doxorubicin and to have gastrointestinal toxicity. The Lewis Y antigen is present in parts of the GI tract and resulted localization of the toxin to the gastric mucosa that is believed to have the GI toxicity. The following references relate to this subject matter: Saleh M. N., et al., “Phase I Trial of the Anti-Lewis Y Drug Immunoconjugate BR96-Doxorubicin in Patients with Lewis Y-Expressing Epithelial Tumors,” J Clinical Oncology, 18(11):2282-2292 (2000); Tolcher,A. W. et al., “Randomized Phase II Study of BR96-Doxorubicin Conjugate in Patients With Metastatic Breast Cancer” J Clinical Oncology, 17(2):478 (1999), the contents of which are incorporated herein by reference in their entirety.

The fundamental problem is that a single factor (in this case the Lewis Y antigen) is not sufficient to distinguish malignant cells from normal cells. Anti-cancer drugs need to be multifactorial. The present invention can enable multifactorial targeting. For example, a drug targeted against cells that only express both the Lewis Y antigen and urokinase would provide exquisite selectivity for breast cancer cells. Urokinase is not present in the GI tract. Targeting specificity alone is often not sufficient to achieve therapeutic effect.

The present invention can be employed to enhance the function of monoclonal antibodies including the ability to:

1.) Enhance the affinity of binding to the target cells;

2.) Enhance the selectivity of target cell binding;

3.) Enhance uptake by target cells;

4.) Allow detoxification by non-target cells;

5.) Overcome multi-drug resistance; and

6.) Ameliorate the problem of antibody inactivation by the binding of soluble antigens.

The present invention encompasses a compound ET; wherein T is a targeting agent that binds or interacts with the target cell or its microenvironment and E is one or more effector moieties that effect the desired chemical, physical, or biological activity; and wherein T is comprised of two or more groups such that each functionality independently and specifically enhances targeting selectivity, affinity, specificity, drug activation, intracellular transport, intracellular localization, or drug detoxification; and wherein one of the groups that comprise T is a monoclonal antibody or targeting receptor binding fragment of a monoclonal antibody, or an analog or derivative thereof which bears amino acid sequence similarity to ligand binding portion of a monoclonal antibody or a fab fragment of an antibody.

A preferred embodiment is comprised of the compound ET in which E is comprised of one or more effector agents having pharmacological activity designated as “PA” and wherein T comprises:

a) A group referred to as a “targeting ligand” which selectively binds to a target receptor on the surface of the target cell or in the microenvironment of the target cell; and wherein this targeting ligand is comprised of: a monoclonal antibody or targeting receptor binding fragment of a monoclonal antibody, or an analog or derivative thereof which bears amino acid sequence similarity to portions of a monoclonal antibody; or a natural protein, or a complex of natural proteins, or a protein, or a naturally occurring polymer; and

b) One or more of the following:

I. A second targeting ligand which selectively binds to a target receptor on the surface of the target cell or in the microenvironment of the target cell;

II. A group, referred to as a “masked intracellular transport ligand” which can be modified in vivo to give a group referred to as an “intracellular transport ligand” which binds to a target cell receptor that actively transports bound ligands into the target cell;

III. A group referred to as a “trigger” that can be modified in vivo, wherein in vivo modification activates the trigger and modulates the pharmacological activity PA;

IV. or a group referred to as a “masked intracellular trapping ligand” which can be modified in vivo to give an “intracellular trapping ligand”; that can bind to one or more type of intracellular receptor;

and wherein if a second targeting ligand is present in T then the first and second targeting ligands are able to bind simultaneously to two targeting receptor molecules;

and wherein if T consists solely of a targeting ligand a trigger and in vivo modification of the trigger increases the pharmacological activity PA then the in vivo modification which activates the trigger is caused by an enzyme or enzymatic activity that is increased at target cells or decreased at non-target cells;

and wherein if T consists solely of a targeting ligand a trigger and in vivo modification of the trigger decreases the pharmacological activity PA then the in vivo modification which activates the trigger is caused by an enzyme or enzymatic activity that is decreased at target cells or increased at non-target cells;

and provided that T is not: an antibody, or an analog or component of an antibody, or a complex of antibodies, or a bispecific antibody, or an analog of a bispecific antibody, or a natural protein, or a complex of natural proteins, or a protein, or a naturally occurring polymer.

Affinity of the targeting complex to the cell can be increased by having a targeting ligand that also binds to a cell associated receptor. Target selectivity can be enhanced and modified by the increased rate and affinity of binding that can occur with the addition of a second binding ligand designated “A2”. This can be especially useful if the density of antigen on the target cells is low while the density of receptors to A2 is high. Binding of a monoclonal antibody to a cell does not insure that effective internalization can take place. Several embodiments of the present invention can be used to insure effective intracellular delivery. One approach is to employ a second targeting ligand A2 that binds to receptors on tumor cells and undergoes receptor mediated endocytosis. The use of a masked intracellular transporter ligand, as discussed previously, can allow for efficient cell uptake without compromising the targeting selectivity. The incorporation of a detoxification trigger allows for the option to selectively inactivate the drug in non-target cells. For example, a monoclonal antibody enzyme conjugate can be targeted to antigens present on critical non-target cells and can selectively detoxify drug at this site. As discussed previously, multi-drug resistance can be overcome by incorporating, in the effector portion of the drug, an inhibitor to P-glycoprotein. The simultaneous coupling of different antineoplastic drugs can also decrease the emergence of drug resistance. The present invention can also be used to ameliorate the problem of soluble antigen interfering with cell binding. The rate at which antigen-antibody binds, is a function of the concentration of the antigen. A second targeting ligand A2 with high affinity for a target cell receptor can increase the concentration of the antibody at the surface of the target cell and consequently increase the rate at which the antibody binds to target cell associated antigen. In addition, even if the antibody has complexed antigen in circulation, A2 can still localize the drug to target cells. Exchange of the soluble antigen with cell bound antigen can be favored by the higher concentration of the antigen on the cell membrane.

The scope of the present invention includes a method to increase the selectivity and binding affinity of monoclonal antibodies, antibody analogs (and other proteins or factors) for targets by coupling to the monoclonal antibody one or more targeting ligands that bind to independent receptors on the intended target and also the method of coupling to the monoclonal antibody one or more groups of the structure E-T. The scope of the present invention also includes the compounds that result from the coupling of ET and an antibody or other protein or natural product that can benefit from the enhanced targeting selectivity, binding affinity, intracellular transport or trapping possible with multifunctional drug delivery vehicles ET of the present invention.

The scope of the present invention also includes a method to increase the intracellular delivery of monoclonal antibodies, antibody analogs (and other proteins or factors) by coupling to the monoclonal antibody a masked intracellular transporter ligand.

Affinity of the targeting complex to the cell can be increased by having a group A2 that also binds to a cell associated receptor. Target selectivity can be enhanced and modified by the increased rate and affinity of binding that can occur with the addition of a second binding ligand A2. This can prove especially useful if the density of antigen on the target cells is low while the density of receptors to A2 is high. Binding of a monoclonal antibody to a cell does not insure that effective internalization can take place. Several embodiments of the present invention can be used to insure effective intracellular delivery. One approach is to employ a second binding ligand A2 that is known to bind to receptors on tumor cells and undergoes receptor mediated endocytosis. The use of a masked intracellular transporter ligand, as discussed previously, can allow for efficient cell uptake without compromising the targeting selectivity. The incorporation of a detoxification trigger allows for the option to selectively inactivate the drug in non-target cells. For example, a monoclonal antibody enzyme conjugate can be targeted to antigens present on critical non-target cells and can selectively detoxify drug at this site. As discussed previously, multi-drug resistance can be overcome by incorporating, in the effector portion of the drug, an inhibitor to P-glycoprotein. The simultaneous coupling of different antineoplastic drugs can also decrease the emergence of drug resistance. The present invention can also be used to ameliorate the problem of soluble antigen interfering with cell binding. The rate at which antigen-antibody binds is a function of the concentration of the antigen. A second receptor A2 with high affinity for a target cell receptor can increase the concentration of the antibody at the surface of the target cell and consequently increase the rate at which the antibody binds to target cell associated antigen. In addition, even if the antibody has complex antigen in circulation, A2 can still localize the drug to target cells. Exchange of the soluble antigen with cell bound antigen can be favored by the higher concentration of the antigen on the cell membrane.

The scope of the present invention includes a method to increase the selectivity and binding affinity of monoclonal antibodies, antibody analogs (and other proteins or factors) for targets by coupling to the monoclonal antibody one or more targeting ligands that bind to independent receptors on the intended target and also the method of coupling to the monoclonal antibody one or more groups of the structure E-T.

The scope of the present invention also includes a method to increase the intracellular delivery of monoclonal antibodies, antibody analogs (and other proteins or factors) by coupling to the monoclonal antibody a masked intracellular transporter ligand.

Linkers

A large variety of chemical structures can be employed as linkers. Considerations for the selection of linkers designated as “L” are as follows:

1) L should have chemical groups that allow it to be covalently coupled to the components of the compound ET. The covalently coupling preferably should not significantly interfere with the function of the attached components;

2) For some but not all embodiments, L should be of sufficient length to allow for crosslinking of targeting receptors;

3) L can preferably be inert in the sense that L should generally not bind with high affinity to cells or tissue components;

4) L should be sufficiently chemically stable to allow the drug to reach its target site functionally intact;

5) L can also have sites to which groups that allow manipulation of drug solubility can be attached; and

6) L preferably should have low immunogenicity.

Linkers with water solubility are especially preferred. Similar requirements apply to linkers used to couple other components of the drug molecule together. The optimal length of the linkers can vary depending on the structure of the receptors. The expected range is from one up to about 350 bond lengths or from to about 10 bond lengths, or from about 10 to about 40 bond lengths, or from about 20 to about 80 bond lengths, or from about 80 to about 150 bond lengths, or from about 150 to about 350 bond lengths, or 1, 2, 3, 4, 5, 6, 7, 8, 9, 10, 11, 12, 13, 14 . . . 350 or about 350 bond lengths; wherein the dots are used to represent the individual numbers in the sequence between 14 and 350. The linkers can be comprised of oligo or poly-ethylene glycols —(O—CH₂—CH2—)n— with (n=1, 2, 3, 4, 5, 6, 7, 8, 9, 10, 11 . . . or 120 or about 120), gycols, oligo or polypropylene glycols, polypeptides, oligopeptides, —(CH2)n—, with (n=1, 2, 3, 4, 5, 6, 7, 8, 9, 10, 11 . . . or 25 or about 25). The linker can have groups that increase water solubility. Preferred embodiments of such groups comprise: phosphates, phosphonates, phosphinates, sulfonates, carboxylates, amines, hydroxy groups, and polyalcohols. The linker can be connected to the other components of ET by a large variety of chemical bonds. Preferred functionalities include, but are not limited to: carboxylate esters and amides, amides, ethers, carbon- carbon, disulfides, -S-S-S-, acetals, esters of phosphates, esters of phosphinates, esters of phosphonates, carbamates, ureas, N—C bonds, thioethers, sulfonamides, and thioureas. Especially preferred are amide bonds.

Linkers can be linear or can be nonlinear with branches. Linkers can be comprised of shorter linkers that are covalently joined. In preferred embodiments the covalent joining is at a multivalent molecule to which multiple linkers can be coupled. The multivalent molecule can be essentially any molecule to which the linkers can be covalently coupled. Preferred embodiments are molecules that have multiple chemical functionalities such as amino, carboxylate, hydroxy, —SH, isocyanate, and isothiocyanate that can be reacted with the linker to form a covalent bond. Preferred embodiments include: L-amino acids, D- amino acids, or racemic mixtures thereof, amino acid analogs, lysine, aspartic acid, cysteine, glutamic acid, serine, homoserine, hydroxyproline, ornithine, tyrosine, glycerol, pentaerithrol, erithol, and citric acid. One skilled in the arts would readily recognize a very large number of other polyfunctional molecules that can be employed to connect smaller linkers together.

Examples of molecules that are suitable for use as linkers or as molecules to join together multiple linkers can be found in the Aldrich Chemical Catalog (2000) of Sigma -Aldrich Co. and the Shearwater Polymers, Inc. Catalog “Functionalized Biocompatible Polymers for Research and Pharmaceuticals. Polyethylene Glycol and Derivatives,” (2000), and Calas M., et al., “Antimalarial Activity of Compounds Interfering with Plasmodium falciparum Phospholipid Metabolism: Comparison between Mono- and Bisquaternary Ammonium Salts,” J Med Chem, 43:505-516 (2000); and Girault S., et al., “Antimalarial, Antitrypanosomal, and Antileishmanial Activities and Cytotoxicity of Bis(9-amino-6-chloro-2-methoxyacridines): Influence of the Linker,” J Med Chem, 43:2646-2654 (2000); and Halazy S., et al., “Serotonin Dimers: application of the Bivalent Ligand Approach to the Design of New Potent and Selective 5-HTAgonists,” J Med Chem, 39:4920-4927 (1996); and Yano K., et al., “Simultaneous Activation of Two Different Receptor Systems by Enkephalin/Neurotensin Conjugates having Spacer Chains of Various Lengths,” Eur J Pharm Sci, 7:41-48 (1998);and Profit A. A., et al., “Bivalent Inhibitors of Protein Tyrosine Kinases,” J Am Chem Soc, 121:280-283 (1999)and Portoghese P. S., et al., “Hybrid Bivalent Ligands with Opiate and Enkephalin Pharmacophores,” J Med Chem, 30:1991-1994 (1987); and Glick G. D., et al., “Ligand Recognition by Influenza Virus,” J Biol Chem, 266(35):23660-23669 (1991); Glick J. D.; Knowles J. R., “Molecular Recognition of Bivalent Sialosides by Influenza Virus,” J Am Chem Soc, 113:4701-4703 (1991);and Blaustein R. O., et al., “Tethered Blockers as Molecular ‘Tape Measures’ for a Voltage-Gated K+ Channel,” Nature Structural Biology, 7(4):309-311 (2000); Tetsui S., et al., “Opioid Receptor Affinity of Multivalent Ligand System Consisting of Polymerized Liposome,” Int J Peptide Protein Res, 48:95-101 (1996); and Sasaki-Yagi Y., et al., “Binding of Enkephalin/Dextran Conjugates to Opioid Receptors,” Int J Peptide Protein Res, 43:219-224 (1994); and ; and Zhao J., et al., “Receptor Affinity of Neurotensin Message Segment Immobilized on Liposome,” Biochimica et Biophysica Acta, 1282:249-256 (1996) and Kane P., et al., “Cross-Linking of IgE-Receptor Complexes at the Cell Surface: Synthesis and Characterization of a Long Bivalent Hapten that is Capable of Triggering Mast Cells and Rat Basophilic Leukemia Cells,” Molecular Immunology,” 23(7):783-790 (1986); and WO 99/53951 10/28/99 Martinez, et al., “Terminally-Branched Polymeric Linkers and Polymeric Conjugates Containing the Same”.; and U.S. Pat. No. 5,783,178 Jul. 21, 1998 and Rose K.; Vizzavona J., “Stepwise Solid-Phase Synthesis of Polyamides as Linkers,” J Am Chem Soc, 121:7034-7038 (1999); and Kramer R. H.; Karpen J. W., “Spanning Binding Sites on Allosteric Proteins with Polymer-Linked Ligand Dimers,” Nature, 395:710-713 (1998); and Fan E., et al., “High-Affinity Pentavalent Ligands of Escherichia coli Heat-Labile Enterotoxin by Modular Structure-Based Design,” J Am Chem Soc, 122:2663-2664 (2000); and Riley A M.; Potter B. V. L., “Poly(ethylene glycol)-linked Dimers of D-myo-inositol 1,4,5-trisphosphate,” Chem Commun, 983-984 (2000); and Rajur S. B., et al., “Hoechst 33258 Tethered by a Hexa(ethylene glycol) Linker to the 5′-Termini of Oligodeoxynucleotide 15-Mers: Duplex Stabilization and Fluorescence Properties,” J Org Chem, 62:523-529 (1997); and Schwabacher A. W., et al., “Desymmetrization Reactions: Efficient Preparation of Unsymmetrically Substituted Linker Molecules,” J Org Chem, 63:1727-1729 (1998); and Bertozzi C. R.; Bednarski M. D., “The Synthesis of Heterobifunctional Linkers for the Conjugation of Ligands to Molecular Probes,” J Org Chem, 56:4326-4329 (1991); and Sépulchre M., et al., “Specific Functionalization of Polyoxirane by Amino, Carboxyl, Sulfo, and Halogeno End Groups,” Makromol Chem, 184:1849-1859 (1983); and Cook R. M., et al., “The Preparation and Synthetic Application of Heterobifunctional Biocompatible Spacer Arms,” Tetrahedron Letters, 35(37):6777-6780 (1994), the contents of which are hereby incorporated by reference in their entirety.

Some preferred embodiments of linkers are shown below:

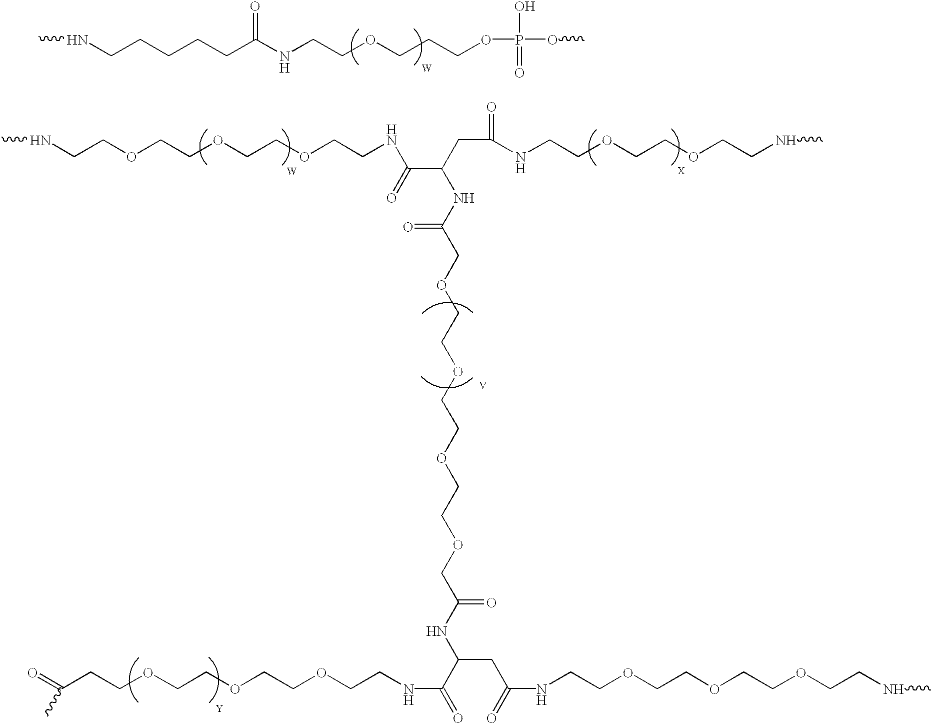

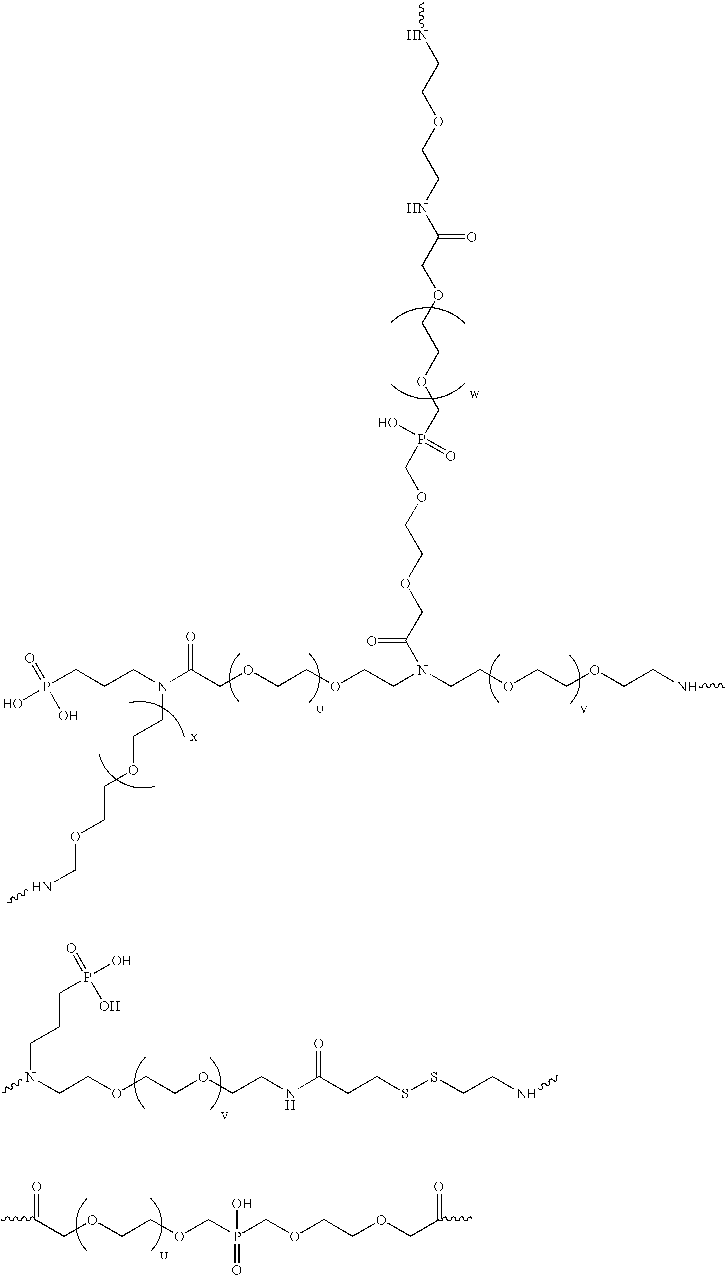

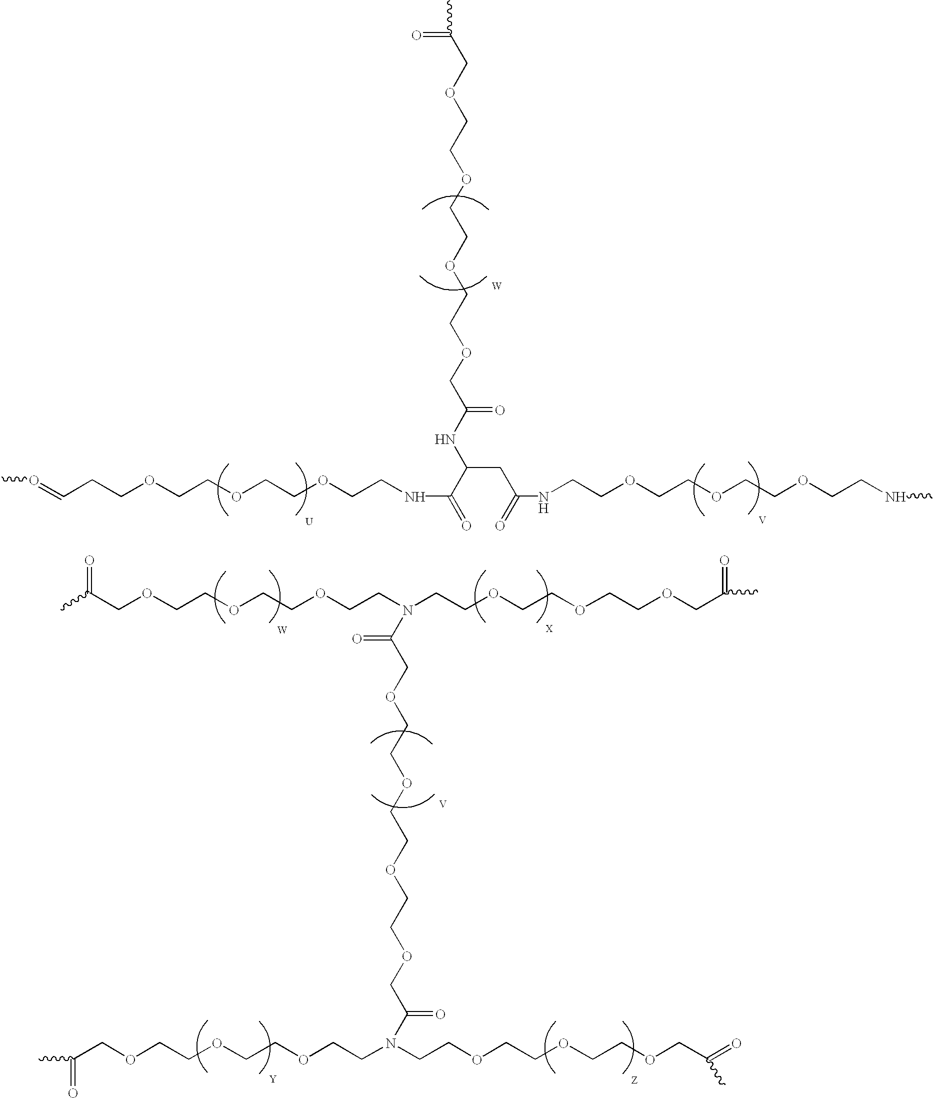

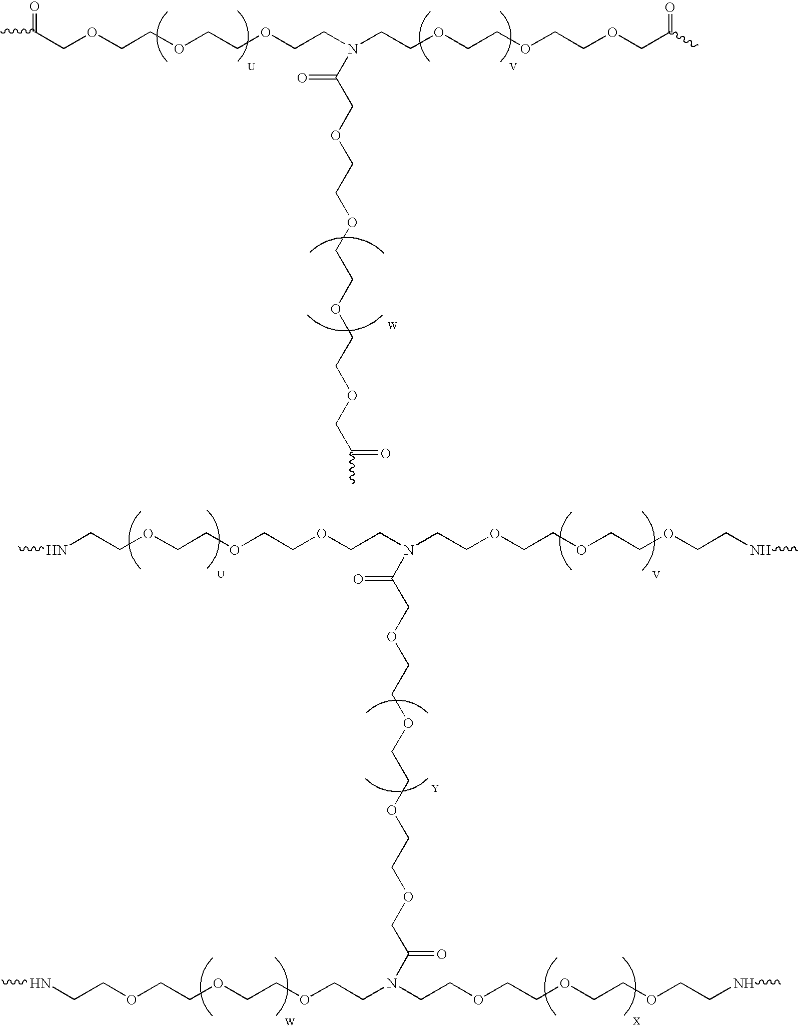

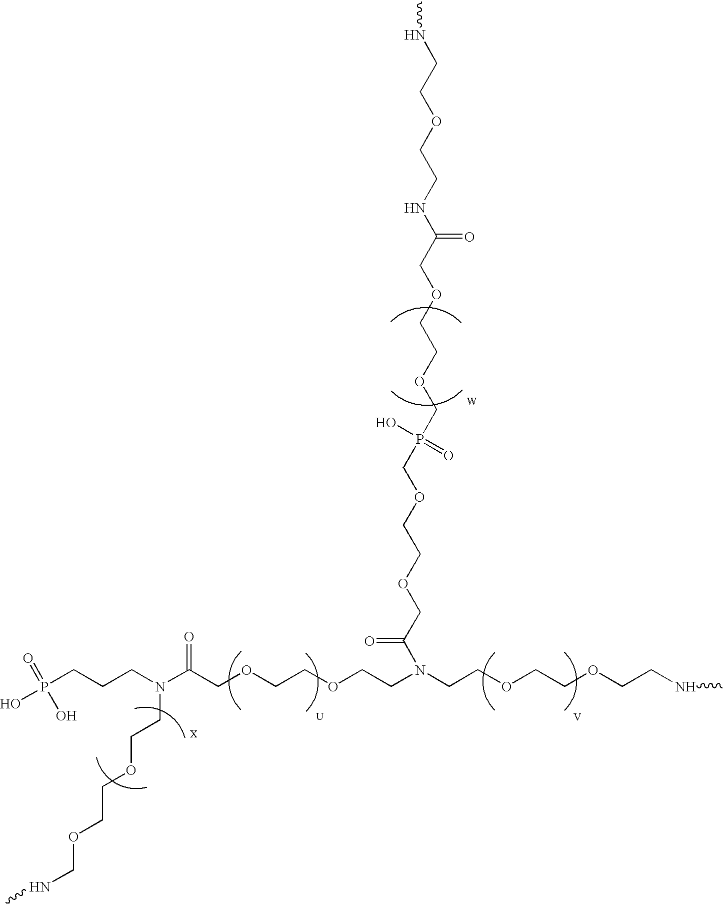

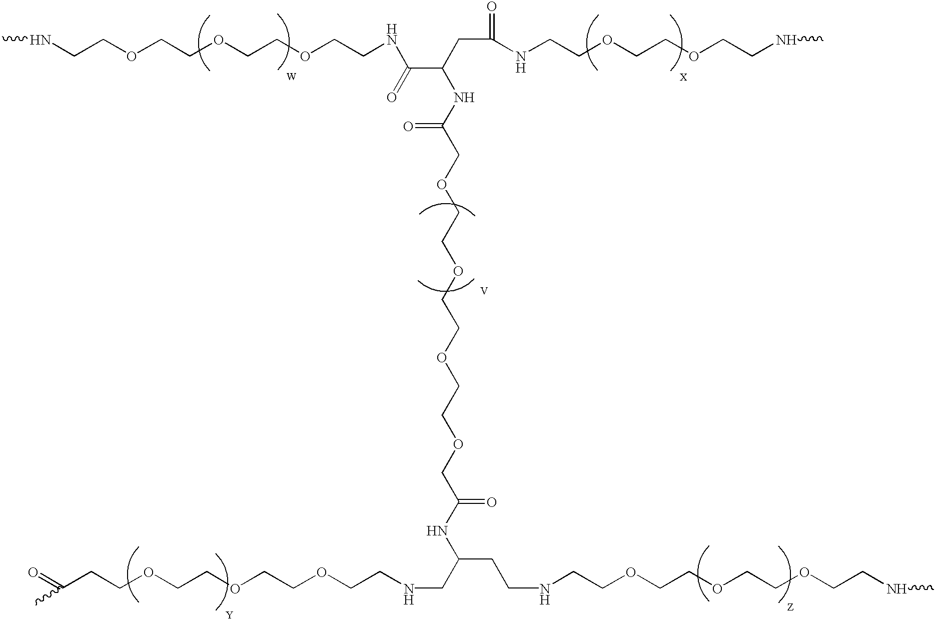

where U=0, 1, 2, 3, 4, 5, 6, . . . 150 or about 150;

where V=0, 1, 2, 3, 4, 5, 6, . . . 150 or about 150;

where w=0, 1, 2, 3, 4, 5, 6, . . . 150 or about 150;

where x=0, 1, 2, 3, 4, 5, 6, . . . 150 or about 150;

where y=0, 1, 2, 3, 4, 5, 6, . . . 150 or about 150;

where z=0, 1, 2, 3, 4, 5, 6, . . . 150 orabout 150;

and wherein the wavy lines are the sites of attachment of the linkers to other components of ET.

Additional preferred embodiments of linkers are comprised of the following structures:

| | |
|---|---|
| 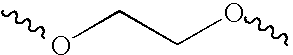 | 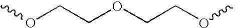 |

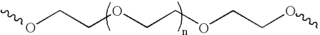

| | |
|---|---|
| 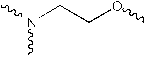 | 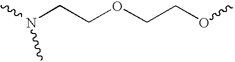 |

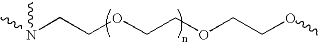

| | |
|---|---|
| 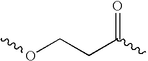 | 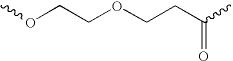 |

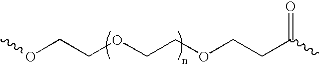

| | |
|---|---|
| 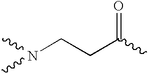 | 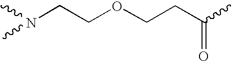 |

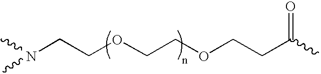

| | |
|---|---|
| 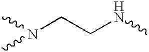 | 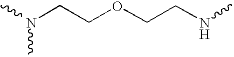 |

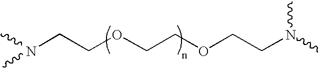

| | |
|---|---|
| 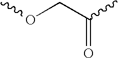 | 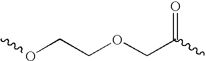 |

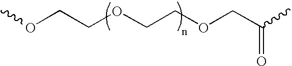

| | |
|---|---|
| 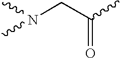 | 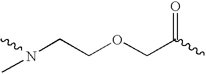 |

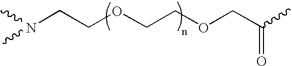

| | |
|---|---|
| 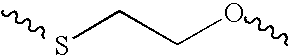 | 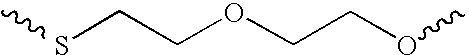 |

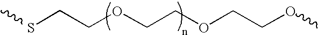

| | |
|---|---|
| 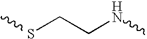 | 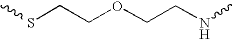 |

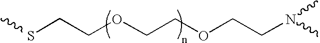

| | | |
|---|---|---|
| 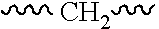 | 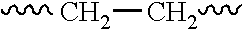 | 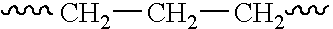 |

| | |
|---|---|
| 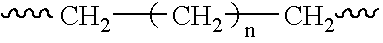 | 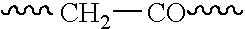 |

| | |
|---|---|
| 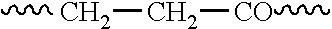 | 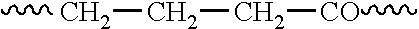 |

| | |
|---|---|
| 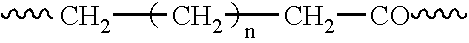 | 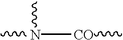 |

| | |
|---|---|
| 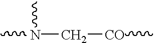 | 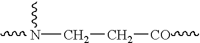 |

| | |
|---|---|
| 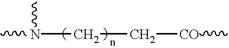 | 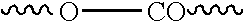 |

| | |
|---|---|
| 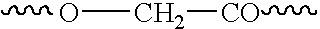 | 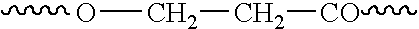 |

| | | |
|---|---|---|
| 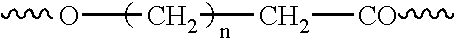 | 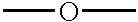 | 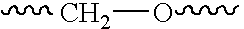 |

| | |
|---|---|
| 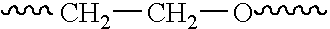 | 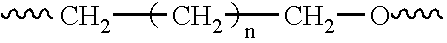 |

| | |
|---|---|
| 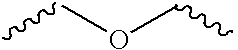 | 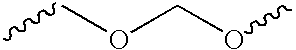 |

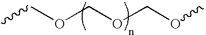

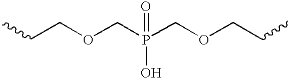

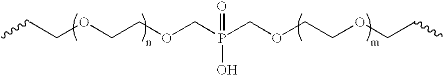

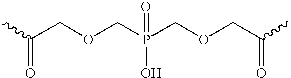

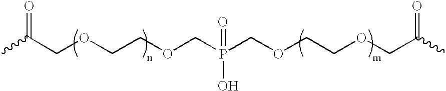

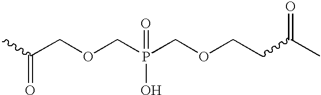

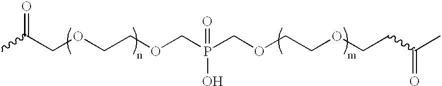

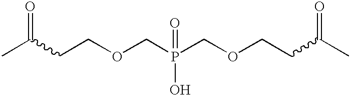

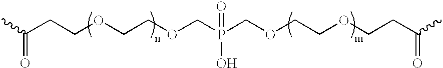

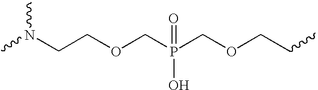

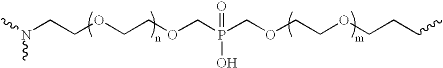

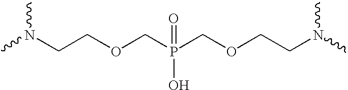

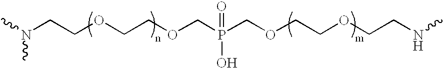

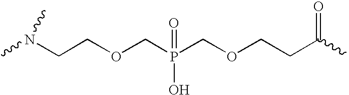

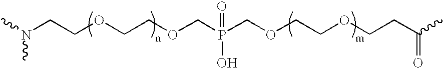

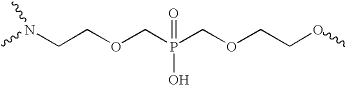

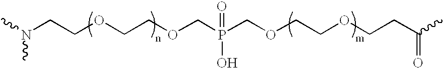

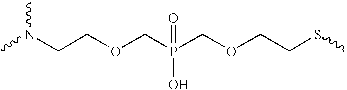

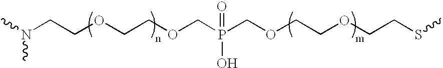

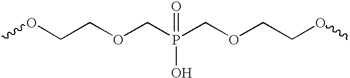

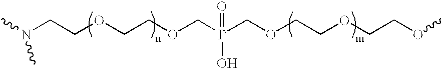

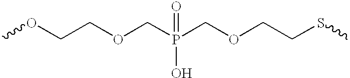

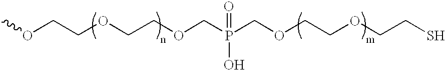

wherein the wavy line is the site of linker attachment to the components of ET or may be H, and wherein m=0, 1, 2, 3, 4, 5, 6, . . . 150 or about 150; and wherein n=0, 1, 2, 3, 4, 5, 6, . . . 150 or about 150;

and wherein the linkers can also be connected to each other or to multifunctional joiner molecules as described above.

Triggers

A wide variety of triggers can be employed in the drug. The function of the triggers is to cause a change in drug function either directly or indirectly by changing the chemistry of the drug upon in vivo modification. Trigger activation can be spontaneous or enzyme catalyzed. Enzyme activated triggers can be non-selective or selective. Selectivity can be for enzymes enriched on target cells or enriched on non-target cells. The triggers can undergo either extracellular or intracellular activation. The triggers can lead to immediate or delayed alteration in drug functionality depending upon the rate of the reaction that is initiated by the triggering event.

The triggers can be attached to the drug in a variety of manners. The key requirements for triggers are as follows:

1.) The trigger can be attached to E-T in a manner that allows for the intended change in drug function upon activation; and

2.) The binding affinity of the trigger, to its activating enzymes, can be much lower than the affinity of the drug to target cells.

In a preferred embodiment, toxifying triggers are designed to undergo cleavage intracellularly and thereby release then free toxins. Intracellular triggers can be activated by a wide range of intracellular enzymes including: hydrolases, proteases, amidases, glycoside hydrolases, thioreductases, Glutathione-S-Transferases, nitroreductases, oxidases, phosphodiesterases, quinone reductases, phosphatases, thiolesterases, oxidoreductases, sulfatases, and esterases.

Note: For the sake of clarity the trigger groups shown in this section include an attached moiety that is released upon trigger activation or trigger function. Strictly speaking, the released group is not part of the trigger group.

In a preferred embodiment (designated TR1) the trigger is comprised of a substituted benzylic analog with a masked or latent electron donating group in the ortho or para positions. Unmasking of this group triggers cleavage of the bond between the benzylic carbon and a leaving group as shown below:

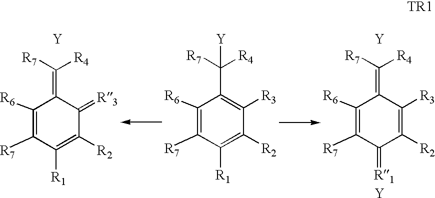

wherein Y is a leaving group and R₁ and R₃, either alone or both, are groups which can be transformed into electron donating groups designated as R″₁ and R″₃ by spontaneous or enzymatic chemical processes; and wherein R₁, R₂, R₃, R₄, R₅, R₆, and R₇ can be a wide range of groups including hydrogen, alkyl groups, halogens, alkoxy, —CO—R₈, where R₈ is OH, an alkyl alkoxy group, or where R₈ can be such that COR₈ comprises an amide. Groups R₁-R₈ also can have an attachment site for a linker attached to the site on the remainder of the drug E-T. A site for linker attachment is optional and is not needed in all embodiments of the invention. If a linker is needed it can be attached in a manner that does not impede trigger function. At least one of the groups R₁ and R₃ must be capable of transformation or biotransformation into an electron donating group. R₁ and R₃ can be an ester, amide, thioester, disulfide, nitro group, H, azido, phosphoester, phosphonoester, phosphinoester, sulfate, alkoxy group, an amino group that is phosphonylated, or phosphorylated and enol ether, an acetal group, a carbonate, or a carbamate. For a detailed discussion of this type of trigger see: Carl, P., “A Novel Connector Linkage Applicable in Prodrug Design,” J Med Chem, 24(5):479-480 (1981); U.S. Pat. No. 5,627,165, May 6, 1997, Glazier, “Phosphorous Prodrugs and Therapeutic Delivery Systems Using Same”; U.S. Pat. No. 5,274,162, Dec. 28, 1993, Glazier, “Antineoplastic Drugs with Bipolar Toxification/Detoxification Functionalities”; U.S. Pat. No. 5,659,061, Aug. 19, 1997, Glazier, “Tumor Protease Activated Prodrugs of Phosphoramide Mustard Analogs with Toxification and Detoxification Functionalities”; Senter, Peter D., et al., “Development of a Drug-Release Strategy Based on the Reductive Fragmentation of Benzyl Carbamate Disulfides,” J Org Chem, 55:2975-2978 (1990), the contents of which are incorporated herein by reference in their entirety.

The table below summarizes some of the groups that are suitable for R₁ and R₃ and the electron donating derivatives into which they are transformed. The mechanisms of the transformation are also shown.

TABLE 1

| | Electron Donating | |
|---|---|---|
| Group R1 or R3 | Derivative | Mechanism |
| esters | Hydroxy, oxy anion | esterases |
| amides | amino | Amidases, proteases |
| thioesters | Thiol, sulfide anion, | Thioesterases, esterases |
| disulfides | Thiol, sulfide anion | Thioreductases |
| nitro | Amino, hydroxyamino | Nitro reductases |
| azido | amino | Azido reductase |
| phosphate | Hydroxy, oxy anion | phosphatases |
| phosphodiesters | Hydroxy, oxy anion | phosphodiesterases |
| phosphonoesters | Hydroxy, oxy anion | phosphodiesterases |
| phospinoesters | Hydroxy, oxy anion | hydrolysis |
| sulfate | Hydroxy, oxy anion | sulfatase |
| alkoxy | Hydroxy, oxy anion | oxidases |
| phosphoramides | amino | Hydrolysis |
| phosphonoamides | amino | hydrolysis |
| enol ether | Hydroxy, oxy anion | hydrolysis |
| acetals | Hydroxy, oxy anion | Acid catalyzed, or |
| | | enzymatic |
| carbonates | Hydroxy, oxy anion | esterases |
| carbamates | amino | Oxidative N-dealkylation |
| hydrogen | Hydroxy, oxy anion | hydroxylation |
| phosphotriester | Hydroxy, oxy anion | See text |

Another preferred embodiment of the trigger utilizes a masked nucleophile which when unmasked catalyzes an intramolecular reaction. The following references relate to this subject matter: Nielsen, N. M. and Bundgaard, H., “Bioreversible Derivatization of Peptides,” Int J Pharm, 29(9):49-68 (1986); Cain, B. F., “2-Acyloxymethylbenzoic Acids. Novel Amine Protective Functions Providing Amides with the Lability of Esters,” J Org Chem, 41(11): 2029-2031 (1976); Chiong, K. N. G., et al., “Rationalization of the Rate of the Acylation Step in Chymotrypsin-Catalyzed Hydrolysis of Amides,” J Am Chem Soc, 97(2):418-423 (1975), the contents of which are incorporated herein by reference in their entirety.

A preferred embodiment of a trigger (TR2) is a group comprised of the following structure:

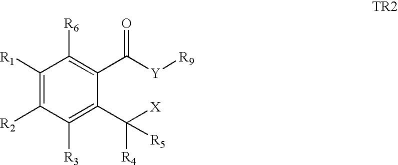

wherein Y is a N bearing group such as NH, NHR7 where R7 is a lower alkyl group which may be substituted with inert groups, or an —S— group, or an —O— group, and HY-R₉ is the compound which is freed upon activation of the trigger, and X is a masked nucleophile and can be a masked amino, hydroxy, or thiol group. R₁-R₆ can be a wide range of groups including: hydrogen, alkyl groups, halogens, Cl, I. F. Br, alkoxy, and —CO—R₈; where R₈ is OH, a lower alkoxy group, or where R₈ can be such that COR₈ comprises an amide. Groups R₁-R₈ also can bear an attachment site for a linker attached to the site on the remainder of the drug. The masked nucleophile X can be any of the groups described in Table 1.

Another preferred embodiment (embodiment TR3) of a trigger is shown below:

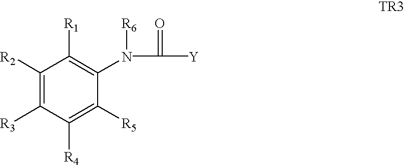

wherein Y is a N bearing group such as NH, R7NH where R7 is a lower alkyl group which may be substituted, or an —S— group, or an —O— group.; wherein R₁, R₂, R₃, and R₄ can be H, a halogen, Cl, F, Br, I, nitro, CH₃, a lower alkyl group, a lower alkoxy group, a sulphonate group, a phosphonate, a phosphate group, or —CO-R₈; where R₈ is OH, a lower alkoxy group, or where R₈ can be such that COR₈ comprises an amide and R₂-R₄ can also be an amino group, a substituted amino group; and HY is the compound released upon trigger activation; R₅ is a masked nucleophile, such as OH, SH, or NH₂, which is masked in a bioreversible fashion;. R₆ is H, or an alkyl group, which can bear inert substituents; wherein R₁-R₆ can have a site of linker attachment to the remainder of the drug complex.

In a preferred embodiment, R₅ is a disulfide, R₂ is H, or nitro, Y is —O—, and R₆ is a linker for attachment to the remainder of the drug complex.

A preferred embodiment (embodiment TR4) of a trigger comprises the following structure:

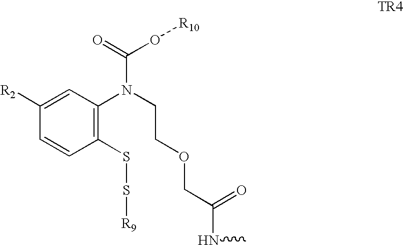

wherein R₂ is H, or nitro; R₉ is any group such that the resulting S—S bond can be reduced by cells to give the corresponding thiol; R₉ can be an alkyl or aryl group, which can bear substituents; and R₉ can be a cysteine or a derivative of cysteine. Substituents on R₉ can include amino, hydroxy, phosphonate, phosphate, or sulfate, which can serve to increase water solubility. R₉ can also be a complex structure such that, both thiol groups that are generated from reduction of the disulfide, each trigger the release of independent drugs. R9 can be a group such as:

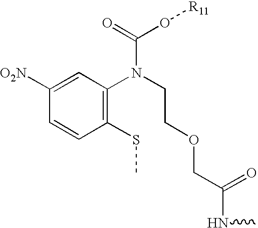

and wherein R₁₀—OH and R₁₁—OH are the compounds that are freed upon activation of the trigger; and wherein the wavy line is the site of attachment of the trigger to the remainder of the drug complex.

Triggers of this class function by a rapid cyclization reaction due to the high effective molarity of the neighboring nucleophile. The following references relate to this subject matter: Hutchins J. E. C.; Fife T. H., “Fast Intramolecular Nucleophilic Attack by Phenoxide Ion on Carbamate Ester Groups,” J Am Chem Soc, 95(7):2282-2286 (1973); and Fife T. H., et al., “Highly Efficient Intramolecular Nucleophilic Reactions. The Cyclization of p-Nitrophenyl N-(2-Mercaptophenyl)-N-methylcarbamate and Phenyl N-(2-Aminophenyl)-N-methylcarbamate,” J Am Chem Soc, 97(20):5878-5882 (1975), the contents of which are incorporated herein by reference in their entirety.

Triggers of this class provide a means of employing a hydroxy group on a drug as the site of trigger attachment, while producing a hydrolytically stable derivative. Triggers of this type can be activated principally inside cells since the concentration of glutathione is approximately 10-30 micromolar in plasma versus 1-10 mM inside cells. Thiol reductase activity is also chiefly intracellular. The following reference relates to this subject matter: Tew K. D., “Glutathione-associated Enzymes in Anti-cancer Drug Resistance,” Cancer Res, 54:4313-4320 (1994), the contents of which is incorporated herein by reference in its entirety.

Another preferred embodiment (TR5) of an intracellular trigger, comprises the following structure:

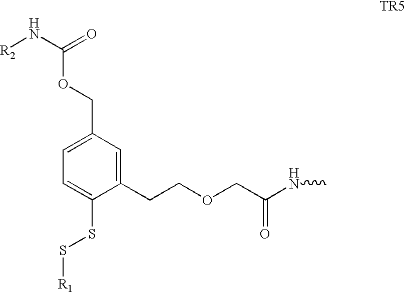

wherein R₁ is a group such that the resulting S—S bond can be reduced by cells to give the corresponding thiol. R₁ can be a lower alkyl or aryl group, which can bear inert substituents. R₁ can be a cysteine or a derivative of cysteine. Substituents on R₁ can include: amino, hydroxy, phosphonate, phosphate, or sulfate groups that increase water solubility. R₁ can also be a complex structure such that both thiol groups, that are generated from reduction of the disulfide, each trigger the release of independent drugs; and wherein R₂—NH₂ is the drug or molecule that is freed upon activation of the trigger; and wherein the wavy line is the site of a linker attachment to the remainder of the drug complex.

Another preferred embodiment (TR6)of an intracellular trigger comprises the structure shown below:

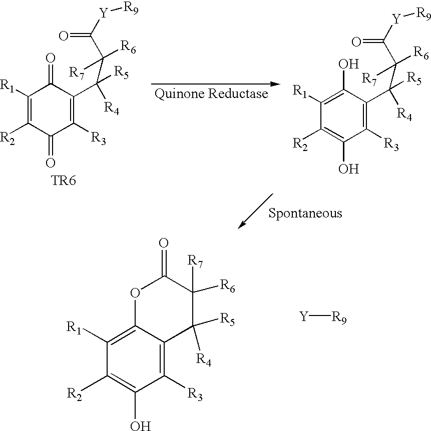

wherein Y is a N bearing group such as NH, or an —O— group, or an —S— group; and wherein HY—R₉ is the compound that is released upon activation of the trigger and R₁-R₇ can be a hydrogen, alkyl groups, halogens, alkoxy, and —CO— R₈; where R₈ is OH; a lower alkoxy group, or where R₈ is such that COR₈ is an amide. Groups R₁-R₇ can bear a site for a linker attached to the site on the remainder of the drug. Triggers of this type are activated by quinone reductases, which function largely intracellularly. The up regulation of quinone reductase by tamoxifen in breast cancer cells is relevant to triggers of this type. The following references relate to this subject matter: Carpino L A, et al., “Reductive Lactonization of Strategically Methylated Quinone Propionic Acid Esters and Amides,” J Org Chem, 54:3303-3310 (1989); and Montano, Monica M.; Katzenellenbogen, Benita S., “The Quinone Reductase Gene: A Unique Estrogen Receptor-Regulated Gene that is Activated by Antiestrogens,” Proc Natl Acad Sci USA, 94:2581-2586 (1997), the contents of which are incorporated herein by reference in their entirety.

Another preferred embodiment (TR7) of intracellular triggers is comprised of the structure shown below:

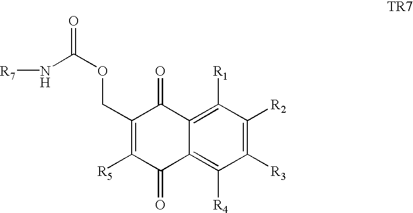

wherein R₁-R₅ can be H, methyl, ethyl, a lower alkyl group, methoxy, a lower alkoxy group, a halogen, Cl, Br, F, I, or —C(O)OR₈; where R₈ is a lower alkyl group, and wherein R₁R₅ can also bear a site of linker attachment to the remainder of the drug complex; and wherein R₇-NH₂ is the compound liberated by trigger activation.

Preferred embodiments (TR8 and TR9) of triggers of this class is shown below:

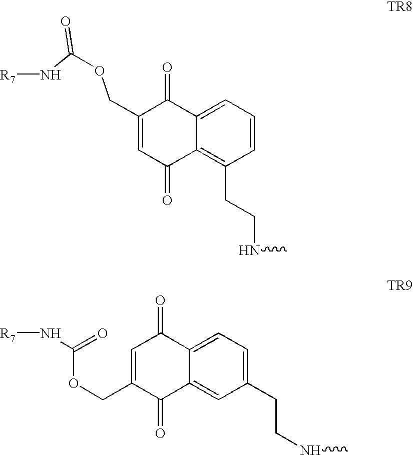

wherein the wavy line is the site of linker attachment. The triggers can be activated intracellularly either by quinone reductase or by nucleophilic addition of glutathione. The following reference relates to this subject matter: Flader C., et al., “Development of Novel Quinone Phosphorodiamidate Prodrugs Targeted to DT-Diaphorase,” J Med Chem, 43:3157-3167 (2000), the contents of which is incorporated herein by reference in its entirety.

Clock-like Time Delayed Triggers

A common theme in multifunctional drug delivery function is localization of the drug to the tumor cells or target cells followed by the activation or unmasking of key components of the drug complex. The timing sequence is important. For example, premature unmasking of a nonspecific intracellular transport ligand can alter the pattern of drug targeting if the intracellular transport ligand has high affinity to its receptor. Clock-like time delayed triggers can allow the drug to have sufficient time to localize to the tumor before the consequences of trigger activation are manifested. The basis of clock-like triggers is that a triggering event initiates a spontaneous chemical reaction that proceeds with a predictable and suitable half-life.

In a preferred embodiment (TR10), the trigger comprises the following structure:

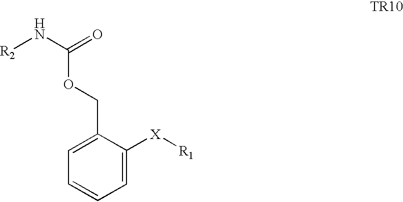

wherein X is O, NH, NCH₃, or S, and R₁ is a bioreversible protecting group which either spontaneously or by enzyme mediated processes is cleaved to unmask —OH, SH, or NH₂, and wherein NH₂—R₂ is the compound that is liberated upon trigger activation.

Ortho positioned electron donating groups promote elimination of benzylic compounds at rates that are slower than the corresponding para derivatives and provide for a time delay clock-like trigger. For example, under conditions in which para thio-benzyl carbamates undergo elimination with a half-life of 10 minutes the corresponding ortho derivative has a half-life of 72 min. Similar behavior is expected for ortho hydroxy, and ortho amino benzylic derivatives. The rate of solvolysis can be adjusted by placing electron-donating or electron withdrawing substituents on the benzylic ring. The following reference relates to this subject matter: Senter, Peter D., et al., “Development of a Drug-Release Strategy Based on the Reductive Fragmentation of Benzyl Carbamate Disulfides,” J Org Chem, 55:2975-2978 (1990), the contents of which is incorporated herein by reference in its entirety.

Other preferred embodiments (TRl1 and TRl2) of a clock-like time delay trigger are comprised of the structures shown below:

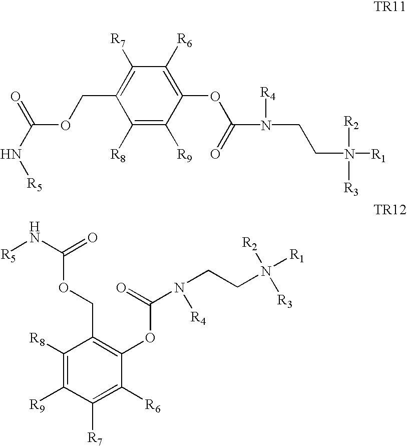

wherein R₁ is a bioreversible amino protecting group; R₂, R₃, and R4 are H, methyl, ethyl, propyl, or a lower alkyl group; and R₆, R₇, R₈ and R₉, are H, a halogen, Cl, Br, I, F, methyl, ethyl, methoxy, or a lower alkoxy group; R₆ and R₉ can be a hydroxy group. Additionally, R₆, R₇, R₈, and R₉ can be the site of linker attachment to the remainder of ET complex; and wherein R₅-NH₂ is the compound that is liberated upon trigger activation.

Activation of these triggers by cleavage of the N—R₁ bond enhances the nucleophilicity of the amino group and initiates a spontaneous cyclization reaction leading to unmasking of the phenolic hydroxy group. The unmasked hydroxy group in turn triggers decomposition of the carbamate group. The half-life of the cyclization reaction can be varied by changing groups R₂- R₄ and R₆-R₉. Increasing steric bulk at R₂, R₃, and R₄ can slow the reaction. Substituents on the phenyl ring that are electron donating and increase the pKa of the corresponding phenol can slow the reaction.

In a preferred embodiment (TR13), R₂ and R₃ are methyl or ethyl and R₁ is an acyl-oxy-methyl group or a phosphono-oxy-methyl group. The resulting positively charged ammonium group cannot participate as effectively in intramolecular catalysis of the carbamate decomposition. Cleavage of the acyl-oxy-methyl group by esterase or of the phosphono-oxy-methyl group by phosphatase can unmask a tertiary amino group. The tertiary amino group can then catalyze the hydrolysis of the carbamate by a cyclic intermediate with a half-life of approximately 40 minutes for the case in which R₂═R₃═R₄═methyl. The following references relate to this subject matter: Saari W. S., et al., “Cyclization-Activated Prodrugs. Basic Carbamates of 4-Hydroxyanisole,” J Med Chem, 33:97-101 (1990); Krise J. P., et al., “Novel Prodrug Approach for Tertiary Amines: Synthesis and Preliminary Evaluation of N-Phosphonooxymethyl Prodrugs,” J Med Chem, 42:3094-3100 (1999); and Krise J. P., et al., “A Novel Prodrug Approach for Tertiary Amines. 3. In Vivo Evaluation of Two N-Phosphonooxymethyl Prodrugs in Rats and Dogs,” J Pharm Sciences, 88(9):928-932 (1999), the contents of which are incorporated herein by reference in their entirety.

Another preferred embodiment (TR14) of a clock-like time delay trigger is comprised of the following structure:

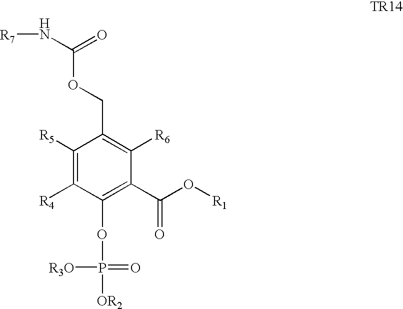

wherein R₁ is a group such that the resulting ester is cleaved either spontaneously or by esterases, and R₂ and R₃ are methyl, ethyl, or lower alkyl groups. R₂ and R₃ can be connected by one or more methylene groups, which can bear inert substituents; and wherein R₄ can be H, OH, methoxy, a lower alkoxy group, methyl, ethyl, or a halogen, or Cl, or F, or I, or Br; . R₅ and R₆ can be H, methoxy, a lower alkoxy group, methyl, ethyl, or Cl, Br, F, I, ; and wherein R₁-R₆ can also bear a site of linker attachment to the remainder of the drug complex; and wherein R₇-NH₂ is the compound liberated by trigger activation.

Triggers of this structure are activated by cleavage of the carboxylic acid ester. The carboxylate group then, by an intramolecular nucleophilic reaction, unmasks the phenolic hydroxy group that in turn initiates decomposition of the carbamate group. The half-life of the intramolecular nucleophilic reaction can be adjusted by varying the nature of the substituents R₂ and R₃. Increasing steric bulk can slow the reaction. Electron donating groups that increase the pKa of the phenolic OH group can also slow the reaction. Steric bulk at R₄ and R₅ can increase the rate. The following reference relates to this subject matter: Bromilow R. H., et al., “Intramolecular Catalysis of Phosphate Triester Hydrolysis. Nucleophilic Catalysis by the Neighbouring Carboxyl Group of the Hydrolysis of Dialkyl 2-Carboxyphenyl Phosphates,” J Chem Soc, 1091-1096 (1971), the contents of which is incorporated herein by reference in its entirety.

A preferred embodiment (TR15) of the above embodiment has the following structure:

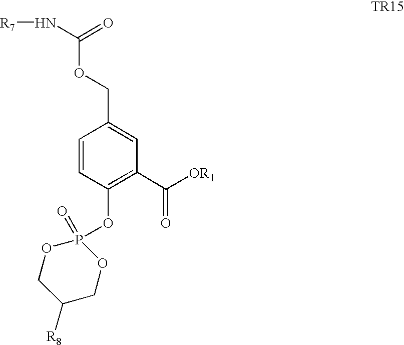

wherein R₈ is H, or O—P(O) (OH)₂. The intramolecular nucleophilic reaction is expected to proceed with a half-life of approximately 90 minutes under physiological conditions for compounds of the above structure with R₈═H.

Other preferred embodiments (TR16 and Tr17) of a clock-like time delay triggers are comprised of the structures shown below:

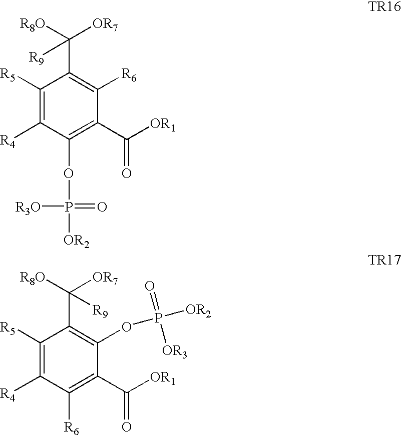

wherein R₁ is a group such that the resulting ester is cleaved either spontaneously or by esterases, and R₂ and R₃ are methyl, ethyl, or lower alkyl groups. R₂ and R₃ can be connected by one or more methylene groups which can bear substituents; and wherein R₄ can be H, OH, methoxy, a lower alkoxy group, methyl, ethyl, or Cl, Br, F, I, R₅ and R₆ can be H, methoxy, a lower alkoxy group, methyl, ethyl, or Cl, Br, F, I, and R₉ can be H, methyl, ethyl, or a lower alkyl group, and wherein R₁-R₆ and R₉ can also bear a site of linker attachment to the remainder of the drug complex; and wherein R₇—OH and R₈—OH are the compounds liberated by trigger activation. R₇ and R₈ can also be connected parts of a single compound, which is released upon trigger activation.

Triggers of this type can be activated by esterase. The resulting carboxylate group can by an intramolecular nucleophilic reaction with the phosphotriester group unmasking the phenolic hydroxy group. The phenolic hydroxy group in equilibrium with the phenolate ion can stabilize carbocation formation at the benzylic carbon and can trigger acetal decompostion.

Another preferred embodiment (TR18) of a clock-like time delay trigger comprises the structure shown below:

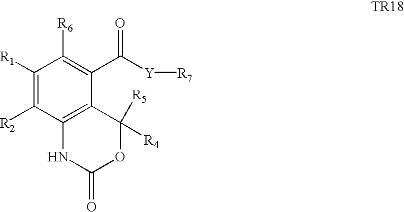

Wherein Y is a N bearing group such as NH, or an —O— group, or an —S— group; and wherein Y—R₇ is the compound that is released upon activation of the trigger; and wherein R₂ and R₆ can be a wide range of groups including: hydrogen, alkyl groups, halogens, alkoxy, and —CO—R₈; where R₈ is OH; or a lower alkoxy group, or where R₈ is selected such that COR₈ is an amide. Groups R₄ and R₅ can be H, an alkyl group, or a phenyl group that can optionally be substituted. Group R₁ is a group of the type described in Table I that can undergo transformation to an electron donating group. Groups R₁-R₆ can optionally bear a site for a linker attached to a site on the remainder of the drug.

This trigger is activated by conversion of R₁ to an electron donating group that initiates cleavage of the benzylic C—O bond. Readdition of the carboxylate group to the benzylic carbon can compete with decarboxylation effectively slowing the rate of carbamate fragmentation as compared to that for noncyclic carbamates. The reactive quinone methide type intermediate can react with water forming a benzylic alcohol. The benzylic alcohol then can undergo intramolecular cyclization and cleave the carboxylate ester or amide functionality releasing Y. The rate can be increased by intramolecular base catalysis via the unmasked meta amino group. The mechansim is shown below:

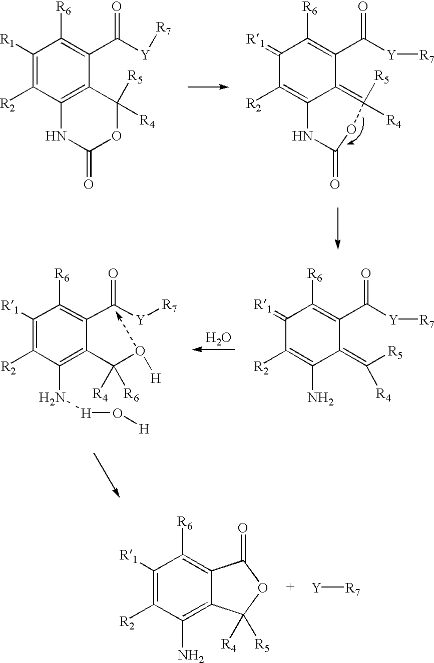

The following reference relates to this subject matter: Fife, Thomas H. and Benjamin, Bruce M., “Intramolecular General Base Catalyzed Alcoholysis of Amides,” J Chem Soc Chem Comm, 14:525-527 (1974), the contents of which is incorporated herein by reference in its entirety.

Triggers can also be strategically placed in groups such as esters, amides, disulfides, acetals, carbonates, and enol ethers which undergo spontaneous or enzymatic transformation and which initiates the intended change in drug function upon activation or in vivo modification.

Detoxification Triggers

Triggers can effect toxification or detoxification of the drug depending upon the particular design. Detoxification triggers can function in a variety of manners. The trigger can impair function of the drug directly or can impair intracellular transport of the drug. For a discussion of detoxification triggers see the following references that relate to this subject matter: U.S. Pat. No. 5,274,162, Dec. 28, 1993, Glazier A., “Antineoplastic Drugs with Bipolar Toxification/Detoxification Functionalities”; and U.S. Pat. No. 5,659,061, Aug. 19, 1997, GlazierA., “Tumor Protease Activated Prodrugs of Phosphoramide Mustard Analogs with Toxification and Detoxification Functionalities”, the contents of which are incorporated herein by reference in their entirety.

In a preferred embodiment of the present invention the detoxification trigger functionally detoxifies the drug by uncoupling the drug from the targeting intracellular transport functionalities. In one embodiment the detoxifier trigger cleaves the active drug coupled to a linker which functions to prevent non-selective cellular uptake. Ionic compounds diffuse very poorly through cell membranes and can be employed in the linker for this purpose.

The factors that trigger detoxification can be specific or non-selective. As discussed previously, a preferred embodiment consists of a drug in which the detoxification trigger can be activated by an enzyme that is selectively delivered to vital non-target cells such as bone marrow stem cells. In a preferred embodiment a detoxification trigger can be activated preferentially in non-target locations. A wide range of detoxifying triggers can be used with the approach of targeted delivery of the detoxifying enzyme to non-tumor cells. Considerations for a selectively targeted detoxification enzyme are as follows:

1.) The enzyme activity deliverable to non-tumor cells can be sufficient to effect detoxification;

2.) The enzyme preferably should be of low toxicity to the normal cells;

3.) The enzyme preferably should not stimulate an autoimmune response against the normal cells;

4.) The affinity of the detoxifying enzyme for the detoxifying trigger preferably should be lower than the affinity of the drug to the targeted tumor cells;

5.) The targeted enzyme preferably should be retained by the cells on the cell surface and not be rapidly internalized;

6.) The level of detoxifying enzyme activity present in the microenvironment of the tumor cells preferably should be insufficient to impede the delivery of a cytotoxic effect to the tumor cells; and

7.) The enzyme preferably should not be rapidly inhibited by plasma factors.

In a preferred embodiment of the invention the detoxification trigger can be a substrate for aryl sulfatase. Aryl sulfatase activity of the blood is low. For example, bone marrow stem cells are characterized by the presence of the CD34 antigen on the cell membrane. A complex of human aryl sulfatase linked to a humanized monoclonal antibody specific for CD34 could be employed to selectively deliver a detoxifying quantity of aryl sulfatase to protect vital bone marrow stem cells (provided, of course the malignancy is CD34 negative). The following references relate to this subject matter: Civin C. I., et al., “Sustained, Retransplantable, Multilineage Engraftment of Highly Purified Adult Human Bone Marrow Stem Cells In Vivo,” Blood, 88(11): 4102-9 (1996); Hill B., et al., “High-Level Expression of a Novel Epitope of CD59 Identifies a Subset of CD34+ Bone Marrow Cells Highly Enriched for Pluripotent Stem Cells,” Exp Hematol, 24(8):936-43 (1996); Civin C. I., et al., “Highly Purified CD34-Positive Cells Reconstitute Hematopoiesis,” J Clin Oncol, 14(8): 2224-33 (1996); and Civin C. I., et al., “Purification and Expansion of Human Hematopoietic Stem/Progenitor Cells,” Ann NYAcad Sci, 770:91-8 (1995), the contents of which are incorporated herein by reference in their entirety.

The use of a human enzyme and humanized monoclonal antibodies can allow for multiple cycles of therapy without problems related to allergenicity. It is also worth emphazing that the presence of circulating anti-CD34 antibody-aryl sulphatase molecules is not expected to significantly interfere with drug delivery to the targeted tumor cells. The reason for this is that the plasma concentration of the drug can be orders of magnitude lower than the Km for the aryl sulfatase towards the detoxifying trigger. This can be compensated for on bone marrow stem cells by the high enzyme concentration at the membrane surface.

Another application of a detoxification trigger is to serve as a time clock. The detoxification trigger can be selected to be activated by nonspecific mechanisms and to initiate detoxification at a predictable rate. This provides the functional equivalent of exposing cells to a timed pulse of active drug. The quantity of drug that the target cells internalize during that time pulse is a function of the rate of uptake. The rate of uptake is a different parameter than the quantity of drug bound to the cells. For example, if tumor cells internalize the drug much faster due to the cross linking of receptors it can be useful to employ such a detoxification trigger, which serves as a time clock.

The combination of a time clock-like detoxification trigger and a tumor-selective trigger for the masked transport ligand provides unique opportunities to refine targeting specificty. One can combine the selectivity of the targeting mechanism of the drug E-T with the targeting specificity of the tumor-selective activating antibody-enzyme complex to achieve enhanced degrees of selectivity. This embodiment of the present invention consists of:

1.) Selecting the masked transport ligand trigger to be such that it is specifically activated by an enzyme referred to as “EZ”;

2.) Selectively delivering EZ to the tumor cells via a tumor antigen specific antibody-EZ complex (or functional analog thereof); or other target agent;

3.) Selecting the detoxification trigger to be activated by a nonspecific clock-like mechanism which provides a sufficient half-life for target cell associated drug to be toxified by the target cell associated antibody-EZ and internalized; and

4.) Selecting the targeting ligands of the drug to be specific for target cell associated receptors that are inefficiently internalized so that intracellular transport is dependent on the intracellular transport ligand.

Since the ultimate target specificity is defined by both the targeted drug and the targeted activating enzyme neither need to have extraordinary tumor selectivity in order to achieve precision targeting. The role of the detoxification trigger is to provide a time limit to the process and restrict toxicity to those sites with efficient cellular uptake, which can correspond to targeted cells.

Masking Triggers

An important application of triggers is to allow a chemical group of the drug complex to be masked or hidden like a trojan horse until the trigger is activated. Numerous examples are given in other sections. The function of masking triggers is to prevent the modified or masked group of exerting its biological activity until trigger activation. A masking trigger is comprised of a chemical structure covalently coupled to a compound which prevents the binding of that compound to a receptor and wherein activation of the masking trigger by spontaneous or enzymatic processes cleaves the bond or bonds between the masking trigger and the compound thereby restoring receptor binding activity.

Intracellular Trapping Ligands

For most anti-cancer drugs the intracellular concentration is the key determinant of cytotoxicity. This is a function of the rate of drug influx and drug efflux. Many tumors exhibit drug resistance by actively pumping anti-cancer drugs out of the cells. To counteract this outward drug flux current drugs are often given in high doses. However, targeted anti-cancer drugs preferably can be given at ultra-low doses such that the concentration outside the cells is close to zero. Under these conditions diffusion of the drug out of the target cell is favored. The function of intracellular trapping ligands is to prevent drug that is intracellular from escaping to outside the cell.

Intracellular trapping ligands can be tumor-selective or non-selective. If the intracellular trapping ligand binds to a receptor that is selectively enriched in tumor cells, enhance tumor targeting selectivity can result. The intracellular trapping ligand can also function to target the drug to critical intracellular locations such as the nucleus or mitochondria and thereby enhance drug activity. The interaction between the intracellular trapping ligand its receptor can be irreversible or reversible, but with high affinity. A wide range of groups that can be adapted for use as intracellular trapping ligands are described in the neoantigen section of this application.

An intracellular trapping ligand is comprised of a group which has the following properties:

1.) The ligand is able to bind with sufficient and preferably high affinity or irreversibly to one or more intracellular receptors (intracellular structures or components);

2.) The ligand must have a site to which a linker can be attached that does not interfere with receptor binding; and

3.) If the intracellular trapping ligand has significant affinity to extracellular structures than it is preferable to employ a masked intracellular trapping ligand.

A masked intracellular trapping ligand is comprised of an intracellular trapping ligand a masking trigger that is preferentially activated inside cells; such that the masking trigger inactivates or interferes with the ability of the group to bind to its receptor and wherein activation of the intracellular trigger can unmask the functional intracellular trapping ligand. Intracellular triggers described in the trigger section of this document can be used as components of masked intracellular trapping ligands.

Non-selective Intracellular Trapping Ligands

In a preferred embodiment, the intracellular trapping ligand is comprised of a masked functionality, which is able to covalently bind to cellular structures following activation of a trigger. The mechanisms of covalent modification of cellular proteins compatible with this embodiment of the invention include: the reaction of electrophiles with nucleophilic groups such as thiols, amines, and hydroxy groups of the proteins; the reaction of nucleophiles with electrophilic centers in proteins and free radical reactions. It is preferrable to employ groups, which require triggering to unmask the reactivity required for protein modification. This can allow the drug targeting specificity to be defined by the high affinity interaction of the targeting ligands the target receptors rather than by the pattern of nonspecific covalent protein modification. The use of chemically stable drugs, which require triggering to unmask reactivity, also has major practical pharmaceutical advantages. A large number of compounds are known, which require triggering or bioactivation for the unmasking of the chemical reactivity including: phosphoramide mustard analogs, quinone methide precursors, enediynes, and nitroimadazoles.

A preferred embodiment (embodiment IT1) of a non-selective intracellular trapping ligand is comprised of the structure shown below:

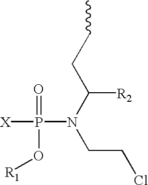

wherein X is NH₂, CH₃NH, (CH₃)₂N, CH₃, C₆C₅, C₆H₅CH₂, a substituted benzyl or a substituted phenyl group, CH₃O, or a lower alkoxy group, and the wavy line is the site of linker attachment to the toxin group, and wherein R₁ is a protecting group which when triggered results in umasking of the free hydroxy group on the phosphorous. R₁ can also bear a site for linker attachment to the remainder of the targeted drug. In this case, the structure R₁ above can serve a dual function of both freeing the toxin‘intracellular trapping ligand from the remainder of the targeted drug complex and activating it towards nucleophilic attack. R₂ is H or CH₂CH₂Cl.

A large number of suitable embodiments of the group R₁ are described in the section on triggers and in U.S. Pat. No. 5,627,165, May 6, 1997 Glazier A., “Phosphorous Prodrugs and Therapeutic Delivery Systems Using Same”. Unmasking of the free OH group on the phosphorous can dramatically increase reactivity towards nucleophiles on adjacent proteins. The conversion of the phosphoester to the negatively charged species enormously increases the nucleophilicity of the adjacent nitrogen and triggers the formation of a highly reactive aziridinium cation, which can rapidly alkylate nucleophiles.

Another preferred embodiment (embodiment IT2) of an intracellular trapping ligand is shown below:

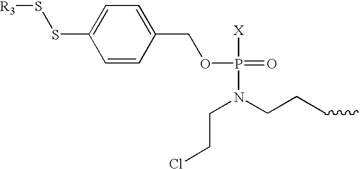

wherein R₃ is a group such that the resulting disulfide can be cleaved intracellularly. In preferred embodiments, R₂—SH is cysteine, an oligopepetide containing cysteine, or an analog of cysteine in which the amino and or carboxylate groups are derivatized.

Intracellular Trapping Ligands Selective for DNA

The site of action of many anti-cancer drugs is on cellular DNA and on nuclear enzymes. In a preferred embodiment, the intracellular targeting ligand is a group that binds to DNA. A large number of agents that bind to DNA are known and can serve the dual function of trapping the drug intracellularly and focusing the drug to site of action at DNA. A preferred embodiment comprised of ethidium homodimer, which binds with high affinity to DNA. The following references relate to this subject matter: Gaugain B., et al., “DNA Bifunctional Intercalators. I. Synthesis and Conformational Properties of an Ethidium Homodimer and of an Acridine Ethidium Heterodimer,” Biochemistry, 17(24):5071-8 (1978); Gaugain B., et al., “DNA Bifunctional Intercalators. 2. Fluorescence Properties and DNA Binding Interaction of an Ethidium Homodimer and an Acridine Ethidium Heterodimer,” Biochemistry, 17(24):5078-88 (1978); Markovits J., et al., “Ethidium Dimer: A New Reagent for the Fluorimetric Determination of Nucleic Acids,” Anal Biochem, 94(2):259-64 (1979); Glazer A. N., et al., “A Stable Double-Stranded DNA-Ethidium Homodimer Complex: Application to Picogram Fluorescence Detection of DNA in Agarose Gels,” Proc Natl Acad Sci, 87:3851-3855 (1990), the contents of which are incorporated herein by reference in their entirety.

In a preferred embodiment (embodiment IT3), the intracellular trapping ligand comprises the following structure:

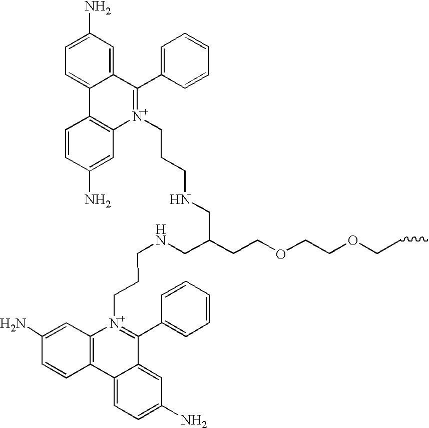

wherein the wavy line is the site of linker attachment to the toxin moiety.

Intracellular Trapping Ligands Selective for Mitochondria

Mitochondria are an important site of action of many anti-cancer drugs. In a preferred embodiment, the intracellular trapping ligand is a group that binds to mitochondrial components. The peripheral benzodiazepam receptor (PBR) is a protein that is localized on the outer mitochondrial membrane and microsomal membranes. Although PBR is widely distributed it is enriched in a variety of tumors. A number of compounds that bind with nanomolar to subnanomolar affinity to PBR are known. The following references relate to this subject matter: Trapani G., et al., “Synthesis and Binding Affinity of 2-Phenylimidazo[1,2-Alpha]Pyridine Derivatives for Both Central and Peripheral Benzodiazepine Receptors. A New Series of High-Affinity and Selective Ligands for the Peripheral Type,” J Med Chem, 40(19):3109-18 (1997); Campiani G., et al., “Synthesis, Biological Activity, and SARs of Pyrrolobenzoxazepine Derivatives, a New Class of Specific “Peripheral-Type” Benzodiazepine Receptor Ligands,” J Med Chem, 39(18):3435-50 (1996); Chaki S., et al., “Binding Characteristics of [3H]DM1106, a Novel and Selective Ligand for Peripheral Benzodiazepine Receptors,” Eur J Pharmacol, 371(2-3):197-204 (1999); Dussossoy D., et al., “Development of a Monoclonal Antibody to Immuno-Cytochemical Analysis of the Cellular Localization of the Peripheral Benzodiazepine Receptor,” Cytometry, 24(1):39-48 (1996); Batra S.; losif C. S., “Elevated Concentrations of Mitochondrial Peripheral Benzodiazepine Receptors in Ovarian Tumors,” Int J Oncol, 12(6):1295-8 (1998); Beinlich A., et al., “Specific Binding of Benzodiazepines to Human Breast Cancer Cell Lines,” Life Sci, 65(20):2099-108 (1999); Venturini I, et al., “Increased Expression of Peripheral Benzodiazepine Receptors and Diazepam Binding Inhibitor in Human Tumors Sited in the Liver,” Life Sci, 65(21):2223-31 (1999); Taketani S., et al., “Involvement of Peripheral-Type Benzodiazepine Receptors in the Intracellular Transport of Heme and Porphyrins,” J Biochem (Tokyo), 117(4):875-80 (1995); Davies L. P., et al., “New Imidazo[1,2-B]Pyridazine Ligands for Peripheral-Type Benzodiazepine Receptors on Mitochondria and Monocytes,” Life Sci, 57(25):PL381-6 (1995); Trapani G., et al., “Novel 2-Phenylimidazo[1,2-A]Pyridine Derivatives as Potent and Selective Ligands for Peripheral Benzodiazepine Receptors: Synthesis, Binding Affinity, and in Vivo Studies,” J Med Chem, 42(19):3934-41 (1999); Bono F., et al., “Peripheral Benzodiazepine Receptor Agonists Exhibit Potent Antiapoptotic Activities,” Biochem Biophys Res Commun, 265(2):457-61 (1999); Batra S.; losif C. S., “Peripheral Benzodiazepine Receptor in Human Endometrium and Endometrial Carcinoma,” Anti-cancer Res, 29(1A):463-6 (2000); Beinlich A., et al., “Relation of Cell Proliferation to Expression of Peripheral Benzodiazepine Receptors in Human Breast Cancer Cell Lines,” Biochem Pharmacol, 60(3):397-402 (2000); Hardwick M., et al., “Peripheral-Type Benzodiazepine Receptor (PBR) in Human Breast Cancer: Correlation of Breast Cancer Cell Aggressive Phenotype with PBR Expression, Nuclear Localization, and PBR-Mediated Cell Proliferation and Nuclear Transport of Cholesterol,” Cancer Res, 59(4):831-42 (1999); Alenfall J., et al., “Cytotoxic Effects of 125I-Labeled PBZr Ligand PK 11195 In Prostatic Tumor Cells: Therapeutic Implications,” Cancer Lett, 134(2):187-92 (1998); Venturini I., et al., “Up-Regulation of Peripheral Benzodiazepine Receptor System in Hepatocellular Carcinoma,” Life Sci, 63(14):1269-80 (1998); Kozikowski A. P., et al., “Synthesis and Biology of a 7-Nitro-2,1,3-Benzoxadiazol-4-YI Derivative of 2-Phenylindole-3-Acetamide: A Fluorescent Probe for the Peripheral-Type Benzodiazepine Receptor,” J Med Chem, 40(16):2435-9 (1997), the contents of which are incorporated herein by reference in their entirety.

In a preferred embodiment (embodiment IT4), the intracellular trapping ligand is a group that binds to PBR. In a preferred embodiment, the group comprises the following structure:

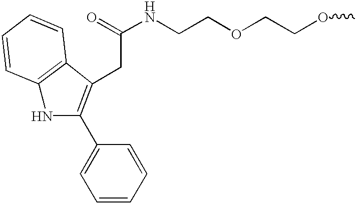

wherein the wavy line is the site of linker attachment to the toxin.

Intracellular Trapping Ligands Selective for Estrogen Receptors

Estrogen receptors (ER) are over-expressed in a number of important human malignancies and can be employed to both trap drugs inside cells and to deliver the drug to the cell nucleus. (See the neoantigen section for a discussion on estrogen receptors and related references.) In a preferred embodiment, the intracellular trapping ligand comprises a group, that binds to estrogen receptors.

In preferred embodiments (embodiments IT5), the intracellular trapping ligand comprises the following structure based on tamoxifen:

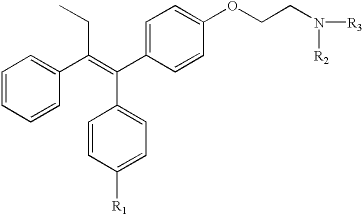

wherein R₁ is H, or OH, or the site of attachment of a trigger connected to the remainder of the targeted drug such that activation of the trigger liberates the tamoxifen analog; and wherein R₂ is H or methyl, and R₃ is the site of linker attachment to the toxin moiety of the drug.

Other preferred embodiments are based on the ability of tamoxifen aziridine and related compounds to irreversibly bind to ER by alkylation of a cysteine residue. The following references relate to this subject matter: Katzenellenbogen J. A., et al., “Efficient and Highly Selective Covalent Labeling of the Estrogen Receptor with [³H]Tamoxifen Aziridine,” J Biol Chem, 258(6):3487-3495 (1983); Harlow K. W., et al., “Identification of Cysteine 530 as the Covalent Attachment Site of an Affinity-labeling Estrogen (Ketononestrol Aziridine) and Antiestrogen (Tamoxifen Aziridine) in the Human Estrogen Receptor,” J Biol Chem, 264(29):17476-17485 (1989); Reese J. C.; Katzenellenbogen B. S., “Mutagenesis of Cysteines in the Hormone Binding Domain of the Human Estrogen Receptor,” 266(17):10880-10887 (1991); Aliau S., et al., “Cysteine 530 of the Human Estrogen Receptor α is the Main Covalent Attachment Site of 11β-(Aziridinylalkoxyphenyl)estradiols,” Biochemistry, 38:14752-14762 (1999), the contents of which are incorporated herein by reference in their entirety.

In these embodiments the intracellular trapping ligand is comprised of an ER binding ligand to which is coupled a latent alkylating agent which is unmasked upon activation of a trigger.

In a preferred embodiment (embodiment IT6 and IT7), the intracellular trapping ligand comprises the following structure:

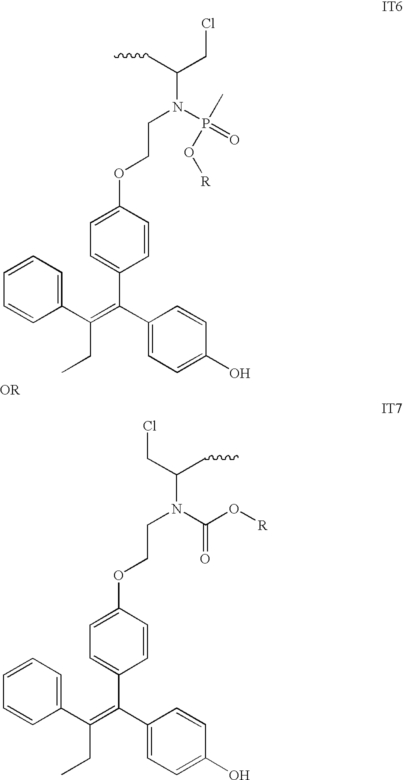

wherein R is a trigger attached to the remainder of the targeted drug such that activation of the trigger cleaves the phophoester or carbamate generating an electrophilic species and wherein the wavy line is the site of linker attachment to the toxin group. A wide variety of suitable triggers have been described elsewhere in this patent. The trigger group R can also bear a site of attachment to the remainder of the targeted drug complex in which case activation of the trigger serves the dual function of both freeing the toxin—intracellular trapping ligand from the remainder of the targeted drug complex and activating it towards nucleophilic attack.

Another preferred set of structures is based on raloxifene. The following references relate to this subject matter: Palkowitz A. D., et al., “Discovery and Synthesis of [6-Hydroxy-3-[4-[2-(1-piperidinyl)ethoxy]phenoxy]-2-(4-hydroxyphenyl)]benzo[b]thiophene: A Novel, Highly Potent, Selective Estrogen Receptor Modulator,” J Med Chem, 40(10):1407-1416 (1997), the contents of which are incorporated herein by reference in their entirety.

A preferred embodiments (embodiments IT8) of an intracellular trapping ligands is comprised of the structure shown below:

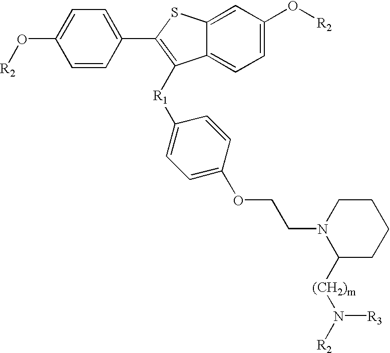

wherein R₁ is CO, CH₂, S, 0, or NH, and m=1 to 6; R₂ is H, or the site of attachment of a trigger connected to the remainder of the targeted drug such that activation of the trigger liberates the raloxifene analog, and R₃ is the site of linker attachment to the toxin group.

Intracellular Trapping Ligands Selective for Fatty Acid Synthase

In a preferred embodiment, the intracellular trapping ligand is a mechanism based enzyme inhibitor of fatty acid synthase, an enzyme that is over-expressed in breast cancer, colon cancer, ovarian, endometrial and prostate cancer. (See the Neoantigen section for discussion and references.)

A preferred embodiment (embodiment IT9) comprises the following structure:

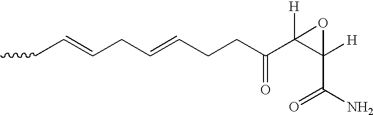

wherein the site of linker attachment to the rest of the drug is indicated by the wavy line.

Intracellular Trapping Ligands Selective for Epidermal Growth Factor Receptor

Epidermal growth factor receptors (EGFR) are membrane associated tyrosine kinases that are over-expressed in a large number of malignancies including: breast, prostate, ovarian, lung, gastric, and bladder. In a preferred embodiment, the intracellular trapping ligand is an irreversible inhibitor to EGFR (and members of the epidermal growth factor receptor family of proteins), that covalently modifies the protein. (See the Neoantigen section for discussion and references.)

In preferred embodiments (embodiments IT10), the intracellular trapping ligand comprises the following structures:

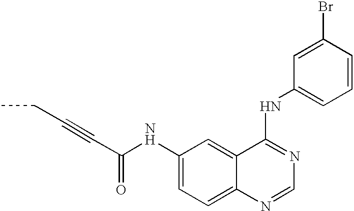

wherein the dotted line is the site of linker attachment to the remainder of the drug.

In other preferred embodiments (embodiment IT11, IT12, IT13, IT14, IT15, IT16, IT17, IT18, IT19, IT20 and IT21), E comprises the following structure:

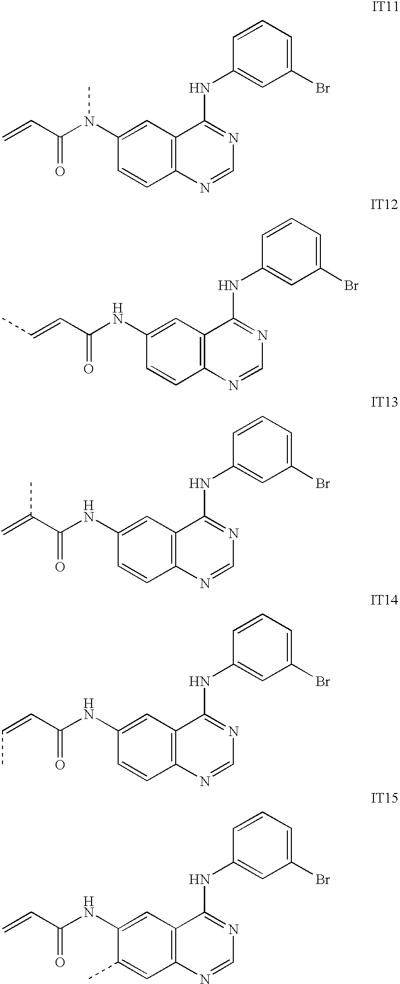

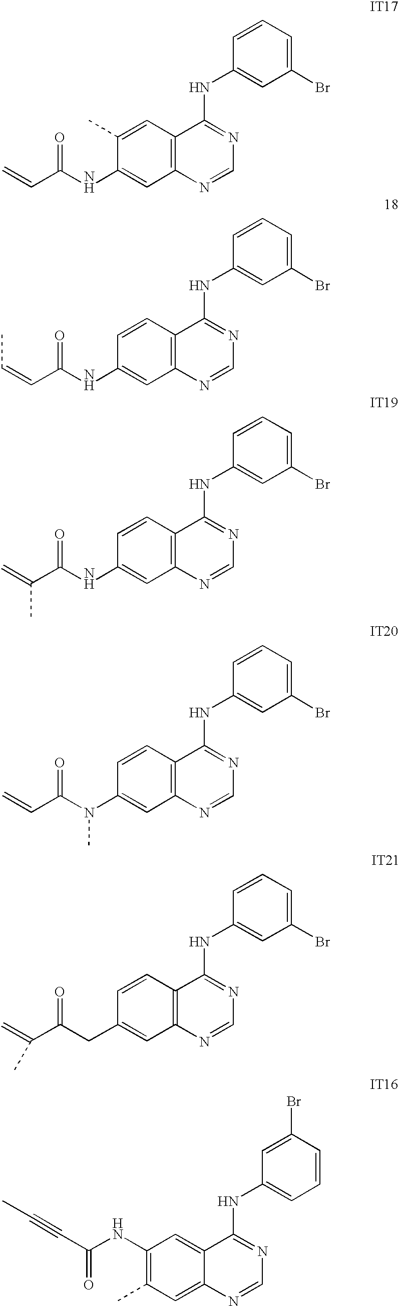

wherein the dotted line is the site of linker attachment to the remainder of the drug.

Intracellular Trapping Ligands Selective for Phospatidylinositol 3-Kinase

Phospatidyl inositol 3-kinase (PIK3) is over-expressed in numerous malignancies including ovarian, breast, prostate, and lung cancer. In a preferred embodiment, the intracellular trapping ligand is an i rreversible inhibitor of PIK3. (See the Neoantigen section for discussion and references.)

A preferred embodiment (emodiment IT22) comprises the following structure:

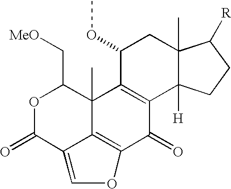

wherein the dotted line is the site of linker attachment to the remainder of the drug and R is O, or OH.

Effector Mechanisms and Effector Agents Diagnostic Applications:

The present invention, E-T, can be employed with an enormous range of effector functionalities E, depending on the intended drug indication.

For diagnostic purposes, a wide E can be comprised of a wide range of entities that allow for detection using imaging techniques commonly employed in radiology and nuclear medicine. The following reference relates to this subject matter:; Reichert D. E., et al., “Metal Complexes as Diagnostic Tools,” Coordination Chemistry Reviews, 184:3-66 (1999); the contents of which is hereby incorporated by reference in its entirety.

Examples include, radioactive moieties, ligands which bind radioisotopes, groups applicable to positron emission tomography, and groups applicable to magnetic resonance imaging, such as gadolinium chelates. The detector group can also be a fluorescent moiety or a group such as biotin, which is amenable to histochemical detection for the applications related to histopathology.

Therapeutic Applications

Although the principle application of this invention is in the area of anti-cancer therapy, the invention can, in principle, be applied to many other areas of drug delivery. For example, the targeting methodology can be used to deliver a cytotoxic agent to a selected class of lymphocytes for the treatment of an autoimmune disease such as scleroderma or lupus erythematosis. The targeting technology can also be used to deliver any therapeutically useful enzyme, protein, or polynucleotide or oligonucleotide.

Anti-cancer Agents

A wide range of anti-cancer drugs can be selectively targeted to tumor cells with the present invention. The high target affinity of the drug E-T for tumor cells can potentially allow a reduction in the total drug dose employed by a factor of 1000 to perhaps 1 million fold compared to non-targeted drug. At these low doses toxicity of the non-targeted drugs generated by metabolism of the targeted drug can be completely inconsequential. However, the targeted drug complex can and potentilly has toxicity defined by the domain of targeting which can never be absolutely specific for tumor cells. Proper selection of the targeted drugs can influence the ultimate therapeutic index as much as selection of the target sites. The optimal situation is when the anti-cancer agent employed has some selective toxicity for tumor cells independent of targeting. Agents which are selectively directed against the mechanisms of cell replication are preferred.

Agents which, mediate toxicity by a single high affinity interaction with a single key enzyme are preferred over drugs with multiple mechanisms of action. For example, if a targeted drug E-T has some affinity for receptors in the heart and the drug delivered is adriamycin (a known cardiotoxin) then cardiotoxicty can result. On the other hand, if the drug delivered is a very selective inhibitor to thymidylate synthetase, (an enzyme nonessential to the heart) then cardiotoxicity is unlikely.

Toxins directed specifically against the key enzymes of cell replication are preferred. These include inhibitors to: thymidylate synthase, DNA polymerase alpha, Toposisomerase I and II, ribonucleotide reductase, Thymidylate kinase, cyclin dependent kinases, DNA primase, DNA helicase, and microtubule function.

Highly preferred embodiments of the invention are with E being comprised of two different anti-cancer drugs or an anti-cancer drug and an inhibitor to p-glycoprotein. Also included within the scope of the present invention is the embodiment in which E is comprised of one or more inhibitors to multi-drug resistance without a coupled toxin. The following references relate to this subject matter: Gottesman Michael M., “How Cancer Cells Evade Chemotherapy” Sixteenth Richard and Hinda Rosenthal Foundation Award Lecture”, Cancer Research, 53:747-754 (1993); Roe M., et al., “Reversal of P-Glycoprotein Mediated Multi-drug Resistance by Novel Anthranilamide Derivatives,” Bioorg Med Chem Lett, 9(4):595-600 (1999); Szakacs G., et al., “Diagnostics of Multi-drug Resistance in Cancer,” Pathol Oncol Res, 4(4):251-7 (1998); Sumizawa T., et al., “Reversal of Multi-drug Resistance-Associated Protein-Mediated Drug Resistance by the Pyridine Analog PAK-104P,” Mol Pharmacol, 51 (3):399-405 (1997); Caner U., “Full Blockade of Intestinal P-Glycoprotein and Extensive Inhibition of Blood-Brain Barrier P-Glycoprotein by Oral Treatment of Mice with PSC833,” J Clin Invest, 10(10):2430-6 (1997); Alexander D., “Histopathological Assessment of Multi-drug Resistance in Gastric Cancer: Expression of P-Glycoprotein, Multi-drug Resistance-Associated Protein, and Lung-Resistance Protein,” Surg Today, 29(5):401-6 (1999); Zhou D. C., et al., “Effect of the Multi-drug Inhibitor GG918 on Drug Sensitivity of Human Leukemic Cells,” Leukemia, 11(9):1516-22 (1997); Courtois A., etal., “Inhibition of Multi-drug Resistance-Associated Protein (MRP) Activity by Rifampicin in Human Multi-drug-Resistant Lung Tumor Cells,” Cancer Lett, 139(1):97-104 (1999); Rappa G., et al., “New Insights into the Biology and Pharmacology of the Multi-drug Resistance Protein (MRP) from Gene Knockout Models,” Biochem Pharmacol, 58(4):557-62 (1999); Kaye S. B., “Multi-drug Resistance: Clinical Relevance in Solid Tumours and Strategies for Circumvention,” Curr Opin Oncol, 10 Suppl 1:S15-9 (1998); Mendez-Vidal C.; Quesada A. R., “Reversal of P-Glycoprotein-Mediated Multi-drug Resistance In Vitro by AV200, a New Ardeemin Derivative,” Cancer Lett, 132(1-2):45-50 (1998); Atadja P., et al., “PSC-833, a Frontier in Modulation of P-Glycoprotein Mediated Multi-drug Resistance,” Cancer Metastasis Rev, 17(2):163-8 (1998); Klopman G., et al., “Quantitative Structure-Activity Relationship of Multi-drug Resistance Reversal Agents,” Mol Pharmacol, 52(2):323-34 (1997); Rabindran S. K., et al., “Reversal of a Novel Multi-drug Resistance Mechanism in Human Colon Carcinoma Cells by Fumitremorgin C,” Cancer Res, 58(24):5850-8 (1998); Dale I. L., et al., “Reversal of P-Glycoprotein-Mediated Multi-drug Resistance by XR9051, a Novel Diketopiperazine Derivative,” Br J Cancer, 78(7):885-92 (1998); Wallstab A., et al., “Selective Inhibition of MDR1 P-Glycoprotein-Mediated Transport by the Acridone Carboxamide Derivative GG918,” Br J Cancer, 79(7-8):1053-60 (1999); Chen G.; Waxman D. J.,” Complete Reversal by Thaliblastine of 490-Fold Adriamycin Resistance in Multi-drug-Resistant (MDR) Human Breast Cancer Cells. Evidence that Multiple Biochemical Changes in MDR Cells Need not Correspond to Multiple Functional Determinants tor Drug Resistance,” J Pharmacol Exp Ther, 274(3):1271-7 (1995); Mistry P., et al., “In vivo Efficacy of XR9051, a Potent Modulator of P-Glycoprotein Mediated Multi-drug Resistance,” Br J Cancer, 79(11-12):1672-8 (1999), the contents of which are incorporated herein by reference in their entirety.

Preferred toxins include: anthracyclines, ellipticines, taxols, mitoxantrones, epothilones, quinazoline inhibitors of thymidylate synthase, stautosporin, podophyllotoxins, bleomycin, aphidicolin, cryptophycin-52, mitomycin c, phosphoramide mustard analogs, vincristine, vinblastine, and indanocine, and compounds with cytotoxicity for cells in the submicromolar range that are currently listed in the U.S. National Cancer Institute's Developmental Therapeutics Program's, Human Tumor Cell Line Screen for Anti-cancer Agents data base which is accessible at http://dtp.nci.nih.gov/. The following references relate to this subject matter: Bisagni E., et al., “Synthesis of 1-Substituted Ellipticines by a New Route to Pyrido[4,3-b]-Carbazoles,” JCS Perkin I, 8(1347):1706-1711 (1978); Martinez E. J., et al., “Phthalascidin, A Synthetic Antitumor Agent with Potency and Mode of Action Comparable to Ecteinascidin 743,” Proc Nati Acad Sci USA, 96:3496-3501 (1999); Dongfang M., et al., “Remote Effects in Macrolide Formation Through Ring-Forming Olefin Metathesis: An Application to the Synthesis of Fully Active Epothilone Congeners,” J Am Chem Soc, 119:2733-2734 (1997); Dai-Shi S., et al., “Structure-Activity Relationships of the Epothilones and the First In Vivo Comparison with Paclitaxel,” Angew Chem Int Ed Engl, 36(19):2093-2096 (1997); Chou Ting-Chao, et al., “Desoxyepothilone B: An Efficacious Microtubule-Targeted Antitumor Agent with a Promising In Vivo Profile Relative to Epothilone B,” Proc Natl Acad Sci USA, 95:9642-9647 (1998); Chou Ting-Chao, et al., “Desoxyepothilone B is Curative Against Human Tumor Xenografts that are Refractory to Paclitaxel,” Proc Natl Acad Sci USA, 95:15798-15802 (1998); Hattori H., et al., “Nucleosides and Nucleotides. 158. 1-(3-C-Ethynyl-β-D-ribo-pentofuranosyl)-cytosine, 1-(3-3-C- Ethynyl-β-D-ribo-pentofu ranosyl)uracil, and Their Nucleobase Analogues as New Potential Multifunctional Antitumor Nucleosides with a Broad Spectrum of Activity,” J Med Chem, 39:5005-5011 (1996); Jesson M. I., et al., “Characterization of the DNA-DNA Cross-Linking Activity of 3′-(3-Cyano-4-morpholinyl)-3′-deaminoadriamycin,” Cancer Res, 49:7031-7036 (1989); Acton E. M., et al., “Intensely Potent Morpholinyl Anthracyclines,” J Med Chem, 27:638-645 (1984); Nagy A., et al., “High Yield 20 Conversion of Doxorubicin to 2-pyrrolinodoxorubicin, and Analog 500-1000 Times More Potent: Structure-Activity Relationship of Daunosamine-Modified Derivatives of Doxorubicin,” Proc Natl Acad Sci USA, 93:2464-2469 (1996); Duch D. S., et al., “Biochemical and Cellular Pharmacology of 1843U89, a Novel Benzoquinazoline Inhibitor of Thymidylate Synthase,” Cancer Res, 53:810-818 (1993); Panda D., et al., “Antiproliferative Mechanism of Action of Cryptophycin-52: Kinetic Stabilization of Microtubule Dynamics by High-Affinity Binding to Microtubule Ends,” Proc Natl Acad Sci USA, 95:9313-9318 (1998); Marsham P. R., et al., “Design and Synthesis of Potent Non-Polyglutamatable Quinazoline Antifolate Thymidylate Synthase Inhibitors,” J Med Chem, 42:3809-3820 (1999); Nicolaou K. C., et al., “Chemical Biology of Epothilones,” Angew Chem Int Ed, 37:2014-2045 (1998); Boger D. L.; Cai H., “Bleomycin: Synthetic and Mechanistic Studies,” Angew Chem Int Ed, 38:448-476 (1999); Leioni L., et al., “Indanocine, a Microtubule-Binding Indanone and a Selective Inducer of Apoptosis in Multi-drug-Resistant Cancer Cells,” J Nat Cancer Inst, 92(3):217-224 (2000), the contents of which are incorporated herein by reference in their entirety.

Preferred cytotoxins to comprise E are compounds that are cytotoxic to cells at low concentrations, preferably at submicromolar or nanomolar concentrations or subnanomolar concentrations. However, in some preferred embodiments even effector agents that are active at micromolar or higher concentrations may be utilized. This is especially true if the effector agent is operative at a cellular compartment that is targeted by the ET drug. Targeting can result in a profound localized increase in concentration of the effector agent and produce localized concentrations thousands to millions of times higher then the overall concentration.

The scope of the present invention also includes the case where E is comprised of a protein, oligopeptide analog, oligonucleotide analog, polynucleotide analog, or other molecular species, which would benefit from the targeted delivery methods.

E can also be comprised of a group, with a therapeutic radioisotope or a boron bearing group, for use in neutron capture therapy. Suitable radioactive agents are well known to one skilled in the arts.

E can be connected to the drug complex either by a trigger, which when activated releases it or E can be connected in a stable fashion directly to a linker. The mode of connection depends upon the requirements for E to exert its effector function. For example, if E is a radioisotope liberation form the target drug complex is unnecessary for activity.

Preferrably the connection of the effector agent to the remainder of the drug ET should be by chemical groups that are sufficiently stable in vivo to allow the drug to reach the target site mostly intact. If the effector agent can evoke its intended pharmacological activity while still attached to the remainder of the molecule ET than it is preferable that the connection of E to T be by a chemical linkage that is resistant or significantly resistant to cleavage in vivo. Examples of preferred chemical linkages for this case include: C-C bonds; ether bonds; amides; carbamates; thioethers; C-N bonds; and ureas.

In a preferred embodiment the effector agent E is a cytotoxic drug that is connected to a trigger that is connected to a linker that is connected to the remainder of the drug ET. In a preferred embodiment the trigger is a group that can be preferentially modified or activated inside cells and releases the cytotoxin inside the cell. Preferred embodiments of triggers are described in the linker section. Other preferred embodiments of triggers are also shown in the Example section. In a preferred embodiment the connection of E to T can be by a chemical linkage that is resistant or significantly resistant to cleavage in vivo but which is cleaved upon in vivo modification or activation of a trigger group. Preferred chemical linkages of an efffector agent to a trigger are by chemical groups such as carbamates, amides, acetals, and ketals, phosphotriesters, phosphonate diesters, and disulfides. Other functionalities such as esters, carbonates, or any other type of chemical linkage that is sufficiently stable in vivo to allow the drug to reach the target site substantially intact may be employed.

Immunological Effector Groups

The present invention can also be used to label target cells for destruction by the immune system. Nature has endowed the body with powerful and effective mechanisms to destroy foreign antigens. The fundamental obstacle to the utilization of these capabilities in the therapy of cancer is the paucity of antigens unique to malignant cells that can trigger an effective immune response. An impressive array of approaches has been utilized to marshal the immune response against tumors with variable results. The following references relate to this subject matter: Vollmer C. M. Jr., et al., “Alpha-Fetoprotein-Specific Genetic Immunotherapy For Hepatocellular Carcinoma,” Cancer Res, 59(13):3064-7 (1999); Gan Y. H., et al., “Antitumour Immunity of Bacillus Calmette-Guerin and Interferon Alpha in Murine Bladder Cancer,” Eur J Cancer, 35(7):1123-9 (1999); Ganss R., et al., “Autoaggression and Tumor Rejection: It takes More than Self-Specific T-Cell Activation,” Immunol Rev, 169:263-72 (1999); Berd D., et al., “Autologous, Hapten-Modified Vaccine as a Treatment for HumanCancers,” Semin Oncol, 25(6):646-53 (1998); Greten T. F.; Jaffee E. M., “Cancer Vaccines,” J Clin Oncol, 17(3):1047-60 (1999); Manzke O., et al., “CD3X Anti-Nitrophenyl Bispecific Diabodies: Universal Immunotherapeutic Tools tor Retargeting T Cells to Tumors.” Int J Cancer, 82(5):700-8 (1999); Vet J. A., et al., “Comparison of P53 Protein Over-expression with P53 Mutation in Bladder Cancer: Clinical and Biologic Aspects,” Lab Invest, 73(6):837-43 (1995); Jager E., et al., “CTL-Defined Cancer Vaccines: Perspectives for Active Immunotherapeutic Interventions in Minimal Residual Disease,” Cancer Metastasis Rev, 18(1):143-50 (1999); Hart D.; Hill G., “Dendritic Cell Immunotherapy for Cancer: Application to Low-Grade Lymphoma and Multiple Myeloma,” Immunol Cell Biol, 77(5):451-9 (1999); Timmerman J. M.; Levy R., “Dendritic Cell Vaccines for Cancer Immunotherapy,” Annu Rev Med, 50:507-29 (1999); Tjoa B. A., et al., “Follow-Up Evaluation Of A Phase II Prostate Cancer Vaccine Trial,” Prostate, 40(2):125-9 (1999); Palmer K., et al., “Gene Therapy with Autologous, Interleukin 2-Secreting Tumor Cells in Patients with Malignant Melanoma,” Hum Gene Ther, 10(8):1261-8 (1999); Takahashi T., et al.,” IgM anti-ganglioside Antibodies Induced by Melanoma Cell Vaccine Correlate with Survival of Melanoma Patients,” J Invest Dermatol, 112(2):205-9 (1999); Riker A., et al., “Immune Selection after Antigen-Specific Immunotherapy of Melanoma,” Surgery, 126(2):112-20 (1999); Harris D. T., et al., “Immunologic Approaches to the Treatment of Prostate Cancer,” Semin Oncol, 26(4):439-47 (1999); Peralta E. A., et al., “Immunotherapy of Bladder Cancer Targeting P53,” J Urol, 162(5):1806-11(1999); McGee J. M., et al., “Melanoma Vaccines as a Therapeutic Option,” South Med J, 92(7):698-704 (1999); Ben-Efraim S., “One Hundred Years of Cancer Immunotherapy: A Critical Appraisal,” Tumour Biol, 20(1):1-24 (1999); Rickinson A. B., “Targeting Human Tumours with Antigen-Specific Cytotoxic T-Cells,” Br J Cancer, 80 Suppl 1:51-6 (1999); McCarty T. M., et al., “Targeting P53 for Adoptive T-Cell Immunotherapy,” Cancer Res, 58(12):2601-5 (1998); Lindauer M., et al., “The Molecular Basis of Cancer Immunotherapy by Cytotoxic T Lymphocytes,” J Mol Med, 76(1):32-47 (1998); Gilliland L. K., et al., “Universal Bispecific Antibody for Targeting Tumor Cells for Destruction by Cytotoxic T Cells,” Proc Nati Acad Sci U S A, 85(20):7719-23 (1998), the contents of which are incorporated herein by reference in their entirety.

The immune system is able to destroy tumors via a number of different mechanisms including: cytotoxic CD8+ lymphocytes, CD4+ lymphocytes, NK cells, activated macrophages, neutrophils, antibody dependent cytotoxicity, activated eosinophils, and gamma/delta T lymphocytes. Antigen specific T cells function as triggers that activate a wide range of antigen nonspecific effectors that can cause profound tissue destruction by antigen nonspecific mechanisms. The importance of nonspecific effector mechanisms in tumor rejection is highlighted by the rejection of MHC II negative melanomas by MHC II restricted CD4+ T cells. The following references relate to this subject matter: Hung K., et al., “The Central Role of CD4+ T Cells in the Antitumor Immune Response,” J Exp Med. 188(12):2357-2368 (1998); Steinman Lawrence, “A Few Autoreactive Cells in an Autoimmune Infiltrate Control a Vast Population of Nonspecific Cells: A Tale of Smart Bombs and the Infantry,” Proc Natl Acad Sci USA, 93:2253-2256 (1996); Greenberg P. D., et al., “Therapy of Disseminated Murine Leukemia with Cyclophosphamide and Immune Lyt-1+,2⁻ T Cells,” J Exp Med, 161:1122-1134 (1985); Mumberg, et al., “CD4⁺ T Cells Eliminate MHC Class II-Negative Cancer Cells In Vivo by Indirect Effects of IFN-γ,” Proc Natl Acad Sci USA, 96:8633-8638 (1999); Qin Z.; Blankenstein T., “CD4+ T Cell-Mediated Tumor Rejection Involves Inhibition of Angiogenesis that is Dependent on IFNγ Receptor Expression by Nonhematopoietic Cells,” Immunity, 12:677-686 (2000), the contents of which are incorporated herein by reference in their entirety.

The immune system has evolved to allow a small number of antigen specific T cells to orchestrate the destructive activities of a large number of nonspecific effector cells. This has the following profound consequences for the targeted immune destruction of tumors:

1.) The targeted delivery or targeted generation in a tumor of a triggering antigen recognized by sensitized T cells can initiate tumor rejection;

2.) The triggering antigen need not be displayed on tumor cells in a form recognizable by antigen specific T cells;

3.) The triggering antigen can be presented to sensitized antigen specific T cells by macrophages and dendritic cells within the stromal compartment of the tumor and initiate tumor rejection; and

4.) Triggering antigens can be derived from intracellular or extracellular factors in the tumor or tumor microenvironment.

The present invention can allow for the immune system to destroy specifically targeted cells. This can be achieved by delivering to the tumor a variety of immunostimulatory molecules including but not limited to:

1.) Masked antigens;

2.) Masked reactive haptens;

3.) Ligands that result in the formation of neoantigens;

4.) Masked ligands for delta/gamma T cell receptors;

5.) Masked ligands that recruit and mobilize macrophages, monocytes and neutrophils; and

6.) Masked ligands that recruit and activate NK cells.

Masked Antigens

The present invention can allow for an intense immune response to be generated against antigens that are completely unrelated to the tumor and for this immune response to be specifically targeted against the tumor. This can be achieved as follows:

1.) The patient is sensitized to the antigen referred to as “AG” so as to generate high levels of cell mediated immunity against cells bearing the antigen AG;

2.) The antigen AG, masked by one or more bioreversible triggers is selectively delivered to the targeted cell;

3.) After localizing to the target cell via high affinity target cell selective ligands the trigger is activated and the antigen AG is unmasked;

4.) The antigen is then processed either by tumor cells or by macrophages in the tumor microenvironment and complexed to MHC I and or MHC II molecules which trigger the activation of sensitized T cells; and

5.) The antigen activated T cells in the tumor trigger tumor destruction by the recruitment and activation of nonspecific effector cells.

A masked antigen is employed in order to prevent the deactivation of lymphocytes by excess free antigen. The following references relate to this subject matter: Butler L. D., et al., “Unresponsiveness in Hapten-Specific Cytotoxic T Lymphocytes,” J Immunol, 131(4):1663-1669 (1983), the contents of which are incorporated herein by reference in their entirety.

A pronounced inflammatory reaction can occur at the target site that can amplify the antitumor activity through the innocent bystander effect and other non-selective mechanisms such as vascular thrombosis. This approach can allow target cell destruction without the use of cytotoxic agents. However, the approach can be used in combination with the administration of targeted cytotoxic drugs. The inflammatory reaction, which accompanies the immune response can increase vascular permeability in the tumor microenvironment and facilitate drug penetration into the tumor. The intensity of the immune response can also be amplified by the concurrent administration of a variety of immunomodulators and cytokines such as Interleukins 2, 4, 6, 7, and 15. The following references relate to this subject matter: Vella A. T., et al., “Cytokine-induced Survival of Activated T Cells In Vitro and In Vivo,” Proc Natl Aced Sci USA, 95:3810-3815 (1998); Ayroldi E., et al., “Interleukin-6 (IL-6) Prevents Activation-Induced Cell Death: IL-2-Independent Inhibition of Fas/fasL Expression and Cell Death,” Blood, 92(11):4212-4219 (1998), the contents of which are incorporated herein by reference in their entirety.

Targets that are present in the tumor microenvironment but not located on the tumor cell surface can also be employed with this embodiment of the invention. Profound local necrosis due to inflammation and vascular occlusion typified by the Arthus reaction could quite effectively mediate tumor cell death. For example, PSMA is present on the neovasculature of a wide range of malignant tumors including: renal, pancreatic, breast, colon, bladder, testicular carcinoma, melanoma, glioblastoma, and soft tissue sarcomas. Selectively delivering a masked antigen to this site can initiate a delayed hypersensitivity reaction, which would be expected to exert considerable antitumor activity. The following references relate to this subject matter: Chang S. S., et al., “Five Different Anti-Prostate-Specific Membrane Antigen (PSMA) Antibodies Confirm PSMA Expression in Tumor-associated Neovasculature,” Cancer Res, 59:3192-3198 (1999), the contents of which are incorporated herein by reference in their entirety.

The trigger to unmask the latent antigen AG can be selective or non-selective. The situation is completely analogous to that discussed for masked intracellular transport ligand triggers. The antigen masking trigger serves the following important roles:

1.) It can allow the drug to localize to the target site prior to the initiation of the immune reaction;

2.) It can prevent desensitization of effector lymphocytes by soluble antigen; and

3.) It can allow the intensity of the immune response to be increased by the systemic administration of additional targeted drug bearing masked antigen during the course of an ongoing immune reaction.

Peptide antigens are recognized by lymphocytes in association with major histocompatibility complex (MHC) molecules. Complex antigenic proteins are degraded to peptide fragments that bind to MHC molecules and trigger lymphocyte activation. The binding of antigenic peptides to both MHC class I and MHC class II molecules can occur either intracellularly or extracellulary. Accordingly, the targeted delivery of an antigenic peptide or complex antigen to tumor cells can result in the binding of that antigen or a portion of the antigen by cellular MHC proteins which can mark the cells for immune destruction. T cells with delta gamma receptors recognize antigens directly in the absence of antigen presentation or complexation to MHC molecules. The targeted delivery of an antigen specific for delta gamma T cells could eliminate the need for antigen processing and complexation to tumor cell MHC molecules. The following references relate to this subject matter: Jondal M., et al., “MHC Class I-Restricted CTL Responses to Exogenous Antigens,” Immunity, 5:295-302 (1996); Schirmbeck R., et al., “Processing of Exogenous Heat-Aggregated (Denatured) and Particulate (Native) Hepatitis B Surface Antigen for Class I-Restricted Epitope Presentation,” J Immunol, 155:4676-4684 (1995); Schirmbeck R.; Reimann J., “‘Empty’ L^{d} Molecules Capture Peptides from Endocytosed Hepatitis B Surface Antigen Particles for Major Histocompatibility Complex Class I-Restricted Presentation,” Eur J Immunol, 26:2812-2822 (1996); Santambrogio L., et al., “Extracellular Antigen Processing and Presentation by Immature Dendritic Cells,” PNAS, 96(26):15056-15061 (1999); Chiu I., et al., “Trafficking of Spontaneously Endocytosed MHC Proteins,” PNAS, 96(24):13944-13949 (1999); Gromme M., et al., “Recycling MHC Class I Molecules and Endosomal Peptide Loading,” Proc Nati Acad Sci USA, 96:10326-10331 (1999); Santambrogio L., et al., “Abundant Empty Class II MHC Molecules on the Surface of Immature Dendritic Cells,” PNAS, 96(26):15050-15055 (1999); Hosken N. A., et al., “Class I-Restricted Presentation Occurs Without Intenalization or Processing of Exogenous Antigenic Peptides,” J Immunol, 142(4):1079-1083 (1989); Jondal M., et al., “MHC Class I-Restricted CTL Responses to Exogenous Antigens,” Immunity, 5:295-302 (1996); Yewdell J. W., et al., “Cells Process Exogenous Proteins for Recognition by Cytotoxic T Lymphocytes,” Science, 239:637-640 (1988); Barlow A. K., et al., “Exogenously Provided Peptides of a Self-antigen Can Be Processed into Forms that Are Recognized by Self-T Cells,” J Exp Med, 187(9):1403-1415 (1998); Hill A; Ploegh H., “Getting the Inside Out: The Transporter Associated with Antigen Processing (TAP) and the Presentation of Viral Antigen,” Proc Natl Acad Sci, 92:341-343 (1995); Schirmbeck R., et al., “Similar as well as Distinct MHC Class I-Binding Peptides are Generated by Exogenous and Endogenous Processing of Hepatitis B Virus Surface Antigen,” Eur J Immunol, 28:4149-4161 (1998); Schirmbeck R., et al., “Injection of Detergent-Denatured Ovalbumin Primes Murine Class I-Restricted Cytotoxic T Cells in Vivo,” Eur J Immunol, 24:2068-2072 (1994); Kovacsovics-Bankowski M., et al., “Efficient Major Histocompatibility Complex Class I Presentation of Exogenous Antigen Upon Phagocytosis by Macrophages,” Proc Nati Acad Sci USA, 90:49424946 (1993); Song R.; Harding C. V., “Roles of Proteasomes, Transporter for Antigen Presentation (TAP), and β₂-Microglobulin in the Processing of Bacterial or Particulate Antigens Via an Alternate Class I MHC Processing Pathway,” J Immunol, 156:4182-4190 (1996); Schirmbeck R., et al., “Processing of Exogenous Heat-Aggregated (Denatured) and Particulate (Native) Hepatitis B Surface Antigen for Class I-Restricted Epitope Presentation,” J Immunol, 155:2676-4686 (1995); Schumacher T. N. M., et al., “Direct Binding of Peptide to Empty MHC Class I Molecules on Intact Cells and In Vitro,” Cell, 62:563-567 (1990); Staerz U. D., et al., “Cytotoxic T Lymphocytes Against a Soluble Protein,” Nature, 329:449-451 (1987); Reimann J., et al., “Alternative Processing Pathways for MHC Class I-Restricted Epitope Presentation to CD8⁺ Cytotoxic T Lymphocytes,” Biol Chem Hoppe-Seyler, 375:731-736 (1994), the contents of which are incorporated herein by reference in their entirety.

A major advantage of the current approach is the ability to generate an intense immune response against an antigen completely unrelated to the tumor and channel this response against the tumor. The present invention can also be used in conjunction with the in vitro sensitization of the patient's lymphocytes, clonal expansion, and subsequent intravenous infusion of the activated lymphocytes into the patient to adoptively transfer the selected immune response.

The present invention can also be used in conjunction with passively administered antibodies directed against the antigen that is masked. Although most antibodies are not directly cytotoxic for tumor cells the antibodies can trigger tumor rejection by cell mediated mechanisms. The following references relate to this subject matter: Dyall R., et al., “Cellular Requirements for the Monoclonal Antibody-mediated Eradication of an Established Solid Tumor,” Eur J Immunol, 29:30-37 (1999); Clynes R., et al., “Fc Receptors are Required in Passive and Active Immunity to Melanoma,” Proc Natl Acad Sci USA, 95:652-656 (1998), the contents of which are incorporated herein by reference in their entirety.

Key requirement for the selective targeted delivery of an antigen to mark a tumor for destruction by the immune system are as follows:

1.) The antigen can be masked in a bioreversible fashion that allows the drug to localize at the target site prior to antigen unmasking. This can be accomplished functionally in a number of ways:

a.) The antigen can be chemically masked by a trigger that is activated specifically or nonspecifically in the tumor microenvironment.

b.) The trigger can be activated by a time clock type trigger, which unmasks the antigen spontaneously at a rate slow enough to allow prior target cell localization.

c.) The trigger can be unmasked by an enzyme specifically and independently targeted to the tumor.

d.) Alternatively, the antigen can be generated at the target site by the very interaction of the targeting ligands with target receptors. For example, tamoxifen binding to the estrogen receptor generates new antigenic determinants. The following reference relates to this subject matter: Martin P. M., et al., “Binding of Antiestrogens Exposes an Occult Antigenic Determinant in the Human Estrogen Receptor,” Proc Natl Acad Sci, 85:2533-2537 (1988), the contents of which is incorporated herein by reference in its entirety.

2.) The masked chemical moiety can have sufficient molecular size and complexity to function as an antigen following unmasking;

3.) The antigen should preferably be capable of evoking a strong immune response;

4.) The antigen should preferably be a foreign chemical species that does not elicit cross reactivity to normal structures;

5.) The antigen preferably should be a distinct subsite of the molecule which can be seperately used to presensitize the individual without risking sensitization to other portions of the drug; and

6.) The antigen needs to have functionalities, which can be masked and which can prevent antigen recognition until unmasking is triggered.

A variety of molecular structures can be employed as a masked antigen. A molecular size comparable to that of an oligopeptide of around 7-8 amino acid groups is required to provide the requisite complexity to elicit a cellular immune response. The following reference relates to this subject matter: Schlossman, S. F; Levine H., “Immunochemical Studies on Delayed and Arthus-Type Hypersensitivity Reactions,” J Immunol, 98(2):211-219 (1967), the contents of which is incorporated herein by reference in its entirety.

Masked Reactive Haptens

A potential limitation of delivering antigens, which require complexation to MHC proteins for immunogenicity, is the polymorphic nature of the MHC proteins which can impart a significant genetic component to the immune response. This can be addressed by delivering a masked reactive hapten to the tumor that can generate multiple types of haptenized oligopeptides. This can be regarded as analogous to the targeted delivery of a masked contact sensitizing agent. The drug binds to the target cells and a reactive hapten is unmasked which covalently modifies cellular proteins. These hapten modified proteins are then processed and complexed to MHC proteins which trigger the activation of hapten specific sensitized T cells. It is likely that a large number of different hapten modified peptides can be complexed to MHC proteins and recognized by cross reacting hapten sensitized CD4+ and CD8+ T cells. In mice, individual CD4+ T cell clones are able to react to haptens attached to MHC class II molecules via multiple different carrier peptides. The extreme sensitivity and amplification possible by this approach is highlighted by data, which indicates that a single hapten molecule on the surface of a target cell can lead to target cell lysis, by hapten specific lymphocytes. The following references relate to this subject matter: Kohler J., et al., “Cross-reactive Trinitrophenylated Peptides as Antigens for Class II Major Histocompatibility Complex-restricted T Cells and Inducers of Contact Sensitivity in Mice. Limited T Cell Receptor Repertoire,” Eur J Immunol, 25:92-101 (1995); Sykulev Y.; Joo M.; Vturina I.; Tsomides T. J.; Eisen H. N.; Evidence that a single peptide-MHC complex on a target cell can elicit a cytolytic T cell response Immunity 6:565-71(1996), the contents of which are incorporated herein by reference in their entirety.

An important feature of this embodiment of the invention is that effector agent E is comprised of one or more groups that are able to covalently modify proteins and components of the tumor. In a preferred embodiment, the chemical reactivity of E is unmasked following the activation of one or more triggers. A large number of chemical entities are able to covalently react with proteins and generate antigenic groups, which can evoke an immune response. Simple haptens such as dinitrophenol can also be coupled to proteins by a large variety of covalent linkers. The mechanisms of covalent modification of cellular proteins compatible with this embodiment of the invention is very broad including the reaction of electrophilies with nucleophilic groups such as thiols, amines, and hydroxy groups of the proteins, the reaction of nucleophiles with electrophilic centers in proteins, and free radical reactions. It is preferrable to employ groups that require triggering to unmask the reactivity required for protein modification. This can allow the drug targeting specificity to be defined by the high affinity interaction of the targeting ligands with the target receptors rather than by the pattern of nonspecific covalent protein modification. The use of chemically stable drugs that require triggering to unmask reactivity also has major practical pharmaceutical advantages. A large number of compounds are known, which require triggering or bioactivation for the unmasking of the chemical reactivity including phosphoramide mustard analogs, quinone methide precursors, enediynes, and nitroimadazoles.

A preferred embodiment (embodiment MRH1) is shown below:

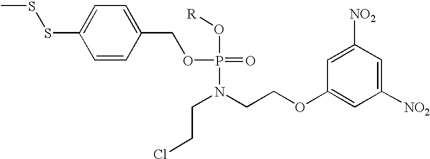

wherein R is the point of linker attachment to the remainder of the target drug complex; cleavage of the disulfide by thiol reductases can release the following compound:

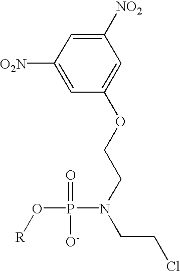

which is an active alkylating agent and can react with nucleophilic groups on adjacent proteins. The conversion of the phosphoester to the negatively charged species enormously increases the nucleophilicity of the adjacent nitrogen and triggers the formation of a highly reactive aziridinium cation, which can rapidly alkylate nucleophiles.

Another preferred embodiment (embodiment MRH2) of E is shown below:

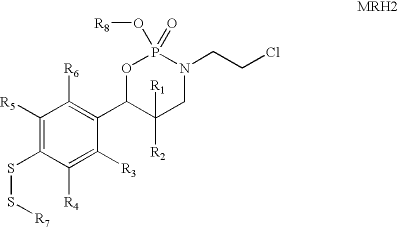

wherein R₁ and R₂ can be H, or lower alkyl group; and R₃ to R₆ can be H, Cl, Br, F, I, a nitro group, a lower alkyl group, C₁-C a methoxy or alkoxy group, —CO₂H, —CO₂R₉, where R₉ is a lower alkyl group, —CONHR₉, —PO₃H₂, —PO₃HR₉, a sulphonic acid group, or other inert groups, which do not interfere with the mechanism of action shown below; and wherein R₇ is an alkyl group, and a phenyl group. R₇—SH can be cysteine or a derivative of cysteine, and R₇ can be a group such that the resulting disulfide is reduced by cells; and wherein R₈ is the point of attachment to the remainder of ET.

The mechanism of protein modification by this group is shown below wherein Nu represents a nucleophilic group on the protein.

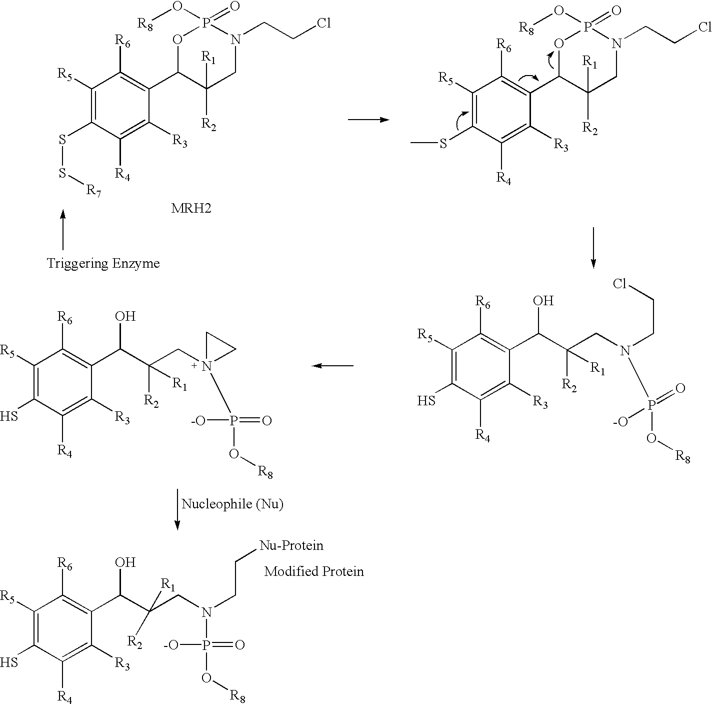

The modified protein can be internalized and degraded by tumor cells and antigen presenting cells such as macrophages in the tumor stroma. The P—N bond is labile and can undergo cleavage. Oligopeptide fragments displaying the hapten shown below can ultimately be presented on the cells in association with MHC I and MHC II molecules.

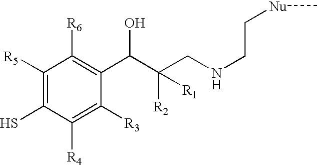

The patient can be sensitized to this hapten without exposure and without sensitizationto the masked hapten. This can be accomplished by immunizing the patient with a compound of the following structure:

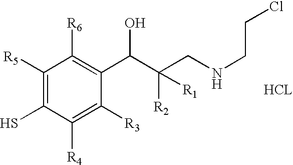

This compound can react with cellular proteins and generate the requisite MHC associated hapten derivatized oligopeptide complexes. The patient may also be immunized with biomolecules such as tumor-associated proteins that have been modified by a compound of the above structure, or fragments, or derivatives of such modified molecules. Methods of sensitization are well known to one skilled in the arts and include:

1.) Topical administration;

2.) Intradermal administration with or without adjuvants; or other immunostimulatory agents;

3.) Administration of dendritic cells exposed in vitro to the haptenizing agent; and

4.) In vitro sensitization of the patients lymphocytes, clonal expansion in vitro, and infusion of the sensitized cells into the patient.

Targeted Neoantigens Formation

Targeted neoantigen formation is a broadly applicable method, which can allow the immune system to be directed against virtually any factor that is over-expressed by tumor cells or by stromal elements with a tumor. There are an enormous number of proteins and enzymes that are enriched in tumor tissues. However, translating the over-expression of a protein or enzyme into toxicity for the tumor is in general not possible with current technologies. In selected cases, inhibition of an overexpresed enzyme can induce cell death. However, this is by no means the rule. In addition, a large number of proteins are enriched in the tumor microenvironment due to over-expression by stromal elements, rather then tumor cells. Examples include a variety of matrix metalloproteinases. Currently no means exist to convert these microenvironmental factors into selective tumor toxicity. Phase III clinical trials of metalloproteinase inhibitors to date have failed to show antitumor efficacy. The following references relate to this subject matter: Basset P.; Okada A.; Chenard M. P.; Kannan R.; Stoll I.; Anglard P.; Bellocq J. P.; Rio M. C., Matrix metalloproteinases as stromal effectors of human carcinoma progression: therapeutic implications. Matrix Biol;15:535-41(1997); Genetic Engineering News, No Anti-cancer Benefit in Trials of Marimastat, Feb. 15, 2000, the contents of which are incorporated herein by reference in their entirety.

In the most general embodiment targeted neoantigen formation consists of the selective generation of neoantigens by the delivery of a drug that irreversibly chemically modifies the target rn. The delivery of a neoantigen forming agent can be by selective or non-selective means. In a preferred embodiment the neoantigen generating effector agent is selectively targeted to the tumor. For certain targets the highly restricted localization of rn can allow for tumor-selective neoantigen formation in the absence of other targeting mechanisms. For example, the uniqueness of a prostatic specific antigen to the prostate can allow for a selective mechanism based suicide inhibitor to PSA to be employed for neoantigen generation and targeted immunotherapy. In this case, the delivery of the PSA selective mechanism based suicide inhibitor need not necessarily be by a targeted multifunctional drug delivery vehicle.

In a preferred embodiment, E is comprised of a group that selectively and irreversibly modifies a target selective receptor rn(s) and generates a neoantigen(s) (AG) to which an immune response can be generated. The selective interaction between the target receptor rn and E confers targeting selectivity to the drug in addition to that provided by the interaction of targeting ligands and target receptors. The molecule rn can be a protein, cellular constituent, or biomolecule either inside, on the surface, or in the microenvironment of tumor cells, which preferably is enriched in the tumor relative to normal tissues.

Table 2 lists some preferred rn for neoantigen formation. It should be noted that the target receptors that bind to the targeting ligands of the drug ET can also be modified and generate neoantigens.

TABLE 2
Preferred Target Receptors Rn for use in Neoantigen Formation

| Enzyme | Malignancies |
|---|---|
| Prostate specific Antigen | prostate, breast |
| Human glandular kallikrein 2 | prostate, breast |
| Prostatic acid phosphatase | prostate |
| Plasmin | numerous |
| Placental type alkaline phosphatase | ovarian, testicular |
| Matriptase | breast |
| Cathepsins | numerous |
| Matrix metalloproteinases | numerous |
| Thymidine phosphorylase | numerous |
| Trypsin | ovarian |
| Urokinase | numerous |
| Fatty Acid Synthase | breast , ovarian, prostate, |
| | endometrial |
| Steroid sulfatase | breast, ovarian, endometrial |
| Epidermal growth factor receptor | numerous |
| Mitogen activated protein kinase | numerous |
| kinase | |
| Phosphatidylinositol 3-kinase | Breast, lung, prostate, ovarian |
| Mitogen activated protein kinase | Breast, prostate, colon, ovarian, |
| | lung |
| Mitogen activated protein kinase | Breast, prostate, ovarian, |
| | endometrial |
| Thymidylate synthase | Colon, cervical, gastric, leukemias, |
| | breast |
| Protein kinase A | Breast, ovarian, lung, colon |
| Fibroblast activation protein/seprase | Stroma of breast, colon, lung, |
| | ovarian, tumor cells of sarcomas |

A general method for converting rn into a neoantigen (AG) (or neoantigen precursor) is to employ a drug E-T in which E has the general structure:

RN—L—V

wherein RN is a group that binds with high affinity to the target rn, and L is a linker, and V is a group that can covalently modify the target rn; and wherein RN and V are linked together in a manner so as to allow RN to retain binding affinity to rn and V to functionally modify rn. In a preferred embodiment, V is activated to a reactive form by a clock-like trigger in which the triggering event is followed by the generation of a reactive intermediate over a predictable time course. In another preferred embodiment V is activated by a trigger that is selectively activated by an enzyme that is enriched at the target cell. The reactive intermediate generated upon activation of V can modify rn and generate neoantigens either by covalently binding to rn or by inducing other covalent changes in rn. For example, V upon activation can generate free radicals that lead to a chemical modification of the target rn. The generation of free radicals in the immediate proximity of the target rn can result in chemical modification of the target. The extreme reactivity of free radicals can enable modification of sites lacking reactive functionalities. The affinity labeling of proteins with azido and diazo compounds that bind and are then activated with ultraviolet light to generate free radical, is a well known biochemical technique. Neoantigens can also be unmasked by the induction breaks in the peptide chain that lead to peptide fragments that are not generated in the normal course of catabolism of the protein. Ene-diyne anti-cancer drugs damage DNA by the generation of a diradical and are also able to react with proteins. Targeted chelating agents have been reported which modify proteins via free radicals generated by the Fenton reaction. The following references relate to this subject matter: Smith A. L.; Nicolaou K. C. “The enediyne Antibiotics,” J Med Chem, 39(11):2103-2117 (1996); Wang K. K., “Cascade Radical Cyclizations via Biradicals Generated from Enediynes, Enyne-Allenes, and Enyne-Ketenes,” Chem Rev, 96:207-222 (1996); Jones G. B., et al., “Understanding Enediyne-Protein Interactions: Diyl Atom Transfer Results in Generation of Aminoacyl Radicals,” Org Lett, 2(6):811-813 (2000); Hoyer D., et al., “A New Strategy for Selective Protein Cleavage,” J Am Chem Soc, 112:3249-3250 (1990); Jones G. B., et al., “Target Directed Enediyne Prodrugs: hER and AhR Degradation by a Synthetic Oxo-Enediyne,” Biorg Med Chem Lett, 6(16):1971-1976 (1996); Schepartz A.; Cuenoud B., et al., “Site-Specific Cleavage of the Protein Calmodulin Using a Trifluoperazine-Based Affinity Reagent,” J Am Chem Soc, 112:3247-3249 (1990); Hirama M., et al., “Synthesis and DNA-Cleaving Abilities of Functional Neocarzinostatin Chromophore Analogues. Base Discrimination by a Simple Alcohol,” J Am Chem Soc, 9851-9853 (1991); Myers A. G.; Proteau P. J., “Evidence for Spontaneous, Low-Temperature Biradical Formation from a Highly Reactive Neocarzinostatin Chromophore-Thiol Conjugate,” J Am Chem Soc, 1146-1147 (1989); Antoniou A. N., et al., “Control of Antigen Presentation by a Single Protease Cleavage Site,” Immunity, 12:391-398 (2000); Casciola-Rosen L., et al., “Scieroderma Autoantigens are Uniquely Fragmented by Metal-catalyzed Oxidation Reactions: Implications for Pathogenesis,” J Exp Med, 185:71-80 (1997); Kalluri R., et al., “Reactive Oxygen Species Expose Cryptic Epitopes Associated with Autoimmune Goodpasture Syndrome,” J Biol Chem, Mar. 23, 2000, the contents of which are incorporated herein by reference in their entirety.

Free radicals can be generated in the course of a variety of cycloaromatization reactions. The following references relate to this subject matter: Hirama M., et al., “Synthesis and Cycloaromatization of a Neocarcinostatin Chromophore Analogue Equipped with an Intramolecular Nucleophile,” Synlett, 651-653 (1991), the contents of which is incorporated herein by reference in their entirety. For example,

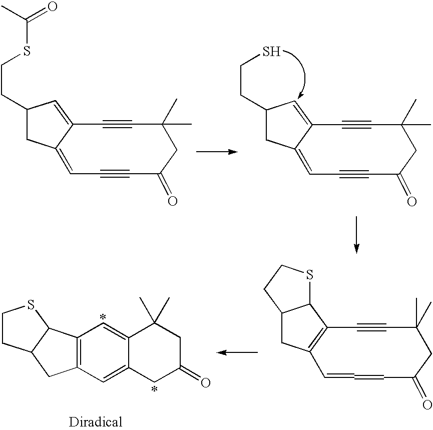

In a preferred embodiment (embodiment V0), V is a triggerable free radical generator.

In a preferred embodiment, (embodiment V1) V comprises the following structure: structure:

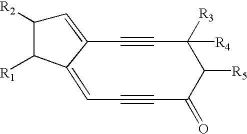

wherein R₁-R₅ can be H, a lower alkyl group or the site of linker attachment to the remainder of the drug; and wherein R₂ can also be grouped with a masked thiol, amino, carboxylate, or other masked nucleophile that can react with the adjacent double bond and form a 3, 4, 5, or 6 membered ring when unmasked.

In preferred embodiments (V2, V3, and V4), V comprises the following structures:

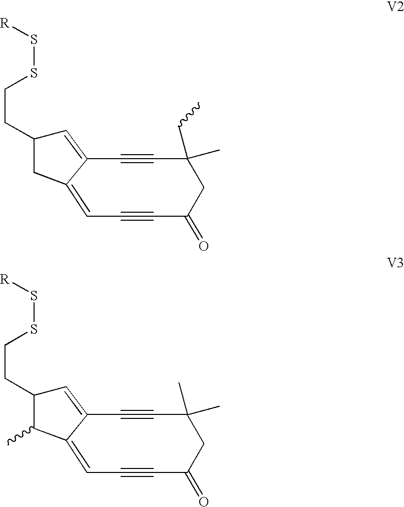

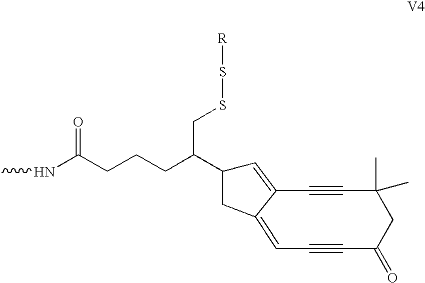

wherein, the dotted line is the site of linker attachement to the remainder of the drug.

A related class of triggerable free radical generators comprises the following structure:

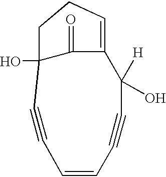

Nucleophilic addition of a thiol triggers a Bergman type cycloaromatization reaction via the intermediacy of a diradical. Compounds of this structure are known to react with proteins. The following references relate to this subject matter: Zein N., et al., “Protein Damage Caused by a Synthetic Enediyne Core,” Biorg Med Chem Lett, 3(6):1351-1356 (1993); Kadow J. F., et al., “Conjugate Addition-Aldol Approach to the Simple Bicyclic-Diynene Core Structure Found in the Esperamicins and Calicheamicins,” Tetrahedron Left, 33(11):1423-1426 (1992), the contents of which are incorporated herein by reference in their entirety.

In a preferred embodiment (V5), V comprises the following structure:

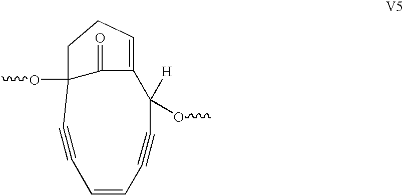

wherein, the wavy line is a linker or H.

In another preferred embodiment, V is a chelating group that binds a metal capable of catalyzing Fenton like reactions and generating hydroxy radicals or other highly reactive radicals. Human tumor cells can produce large amounts of hydrogen peroxide that can generate hydroxy radicals via the Fenton reaction. In addition, under aerobic conditions, the autooxidation of metal complexes can generate hydroxy free radicals. In the presence of the reducing agent ascorbic acid, a redox cycle can be established leading to augmented hydroxy radical production. Ascorbic acid is generated intracellularly by the reduction of dehydroascorbic acid. Dehydroascorbic acid is transported into cells by the Glut 1 and Glut 3 transporter proteins, which are over-expressed in a wide range of malignancies. Accordingly, the combination of a free radical generator, based on a chelating agent ion complex, used in combination with the administration of ascorbic acid or dehydroascorbic acid, can have synergystic activity and enhanced selectivity for Glut 1 and Glut 3 positive tumors. The following references relate to this subject matter: Szatrowski T. P.; Nathan C. F., “Production of large Amounts of Hydrogen Peroxide by Human Tumor Cells,” Cancer Res, 51(3):794-8 (1991); Samuni, A., et al., “On the Cytotoxicity of Vitamin C and Metal Ions,” Eur J Biochem, 137:119-124 (1983); Klebanoff S. J., et al., “Oxygen-based Free Radical Generation by Ferrous Ions and Deferoxamine,” J Biological Chem, 264(33):19765-19771 (1989); Ito, T., et al., “Expression of Facilitative Glucose Transporter Isoforms in Lung Carcinomas: its Relation to Histologic Type, Differentiation Grade, and Tumor Stage,” Mod Pathol, 11(5):437-43 (1998); Baer S. C., et al., “Expression of the Human Erythrocyte Glucose Transporter Glut1 in Cutaneous Neoplasia,” J Am Acad Dermatol, 37(4):575-7 (1997); Younes M., et al., “Over-expression of Glut1 and Glut3 in Stage I Nonsmall Cell Lung Carcinoma is Associated with Poor Survival,” Cancer, 80(6):1046-51 (1997); Younes M., et al., “GLUT1 Expression in Human Breast Carcinoma: Correlation with Known Prognostic Markers,” Anti-cancer Res, 15(6B):2895-8 (1995); Haber R. S., et al., “GLUT1 Glucose Transporter Expression in Colorectal Carcinoma: A Marker for Poor Prognosis,” Cancer, 83(1):34-40 (1998); Burstein D. E., et al., “GLUT1 Glucose Transporter: A Highly Sensitive Marker of Malignancy in Body Cavity Effusions,” Mod Pathol, 11(4):392-6 (1998); Younes M., et al., “Immunohistochemical Detection of Glut3 in Human Tumors and Normal Tissues,” Anti-cancer Res, 17(4A):2747-50 (1997); Grover-McKay M., et al., “Role for Glucose Transporter 1 Protein in Human Breast Cancer,” Pathol Oncol Res, 4(2):115-20 (1998); Younes M., et al., “Wide Expression of the Human Erythrocyte Glucose Transporter Glut1 in Human Cancers,” Cancer Res, 56(5):1164-7 (1996), the contents of which are incorporated herein by reference in their entirety.

Iron (II) complexes with chelating agents are known to generate free radicals under a variety of conditions. The following references relate to this subject matter: Kocha T., et al., “Hydrogen Peroxide-mediated Degradation of protein: Different Oxidation Modes of Copper- and Iron-dependent Hydroxyl Radicals on the Degradation of Albumin,” Biochem Biophys Acta, 1337:319-326 (1997); Egan T. J., et al., “Catalysis of the Haber-Weiss Reaction by Iron-Diethylenetriaminepentaacetate,” J Inorg Biochem, 48:241-249 (1992); Hertzberg R. P.; Dervan P. B., “Cleavage of DNA with Methidiumpropyl-EDTA-Iron (II): Reaction Conditions and Product Analyses,” Biochemistry, 23:3934-3945 (1984); Schepartz A.; Cuenoud B., “Site-Specific Cleavage of the Protein Calmodulin Using a Trifluoperazine-Based Affinity Reagent,” J Am Chem Soc, 112:3247-3249 (1990), the contents of which are incorporated herein by reference in their entirety.

In a preferred embodiment (V6), V is iron complexed with a chelating agent. In a preferred embodiment (V7), V comprises the following structure:

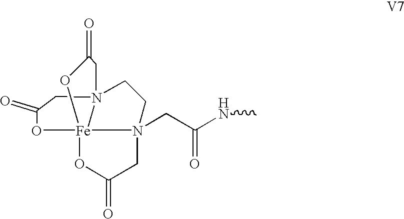

wherein the wavy line is the site of linker attachment to RN.

Salen copper and salen iron complexes are known to generate free radicals under a variety of conditions. The presence of ortho or para hydroxy substituents on the salicylidene moieties leads to a radical generating system from oxygen. The hydroxy substituted salicylidene moieties form hydroquinones, that cooperate in the redox reaction and aid in the generation of free radicals. Intracellularly a variety of mechanisms exist that can lead to redox cycling and the continued generation of free radicals. The following references relate to this subject matter: Lamour E., et al., “Oxidation of Cu^{II} to Cu^{III}, Free Radical Production, and DNA Cleavage by Hydroxy-Salen-Copper Complexes. Isomeric Effects Studied by ESR and Electrochemisty,” J Am Chem Soc, 121:1862-1869 (1999); Routier S., et al., “DNA Cleavage by Hydroxy-Salicylidene-Ethylendiamine-Iron Complexes,” Nucleic Acids Res, 27(21):4160-4166 (1999); Routier S., et al., “Synthesis of a Functionalized Salen-Copper Complex and Its Interaction with DNA,” J Org Chem, 61:2326-2331 (1996); Routier S., et al., “Synthesis, DNA Binding, and Cleaving Properties of an Ellipticine-Salen Copper Conjugate,” Bioconjugate Chem, 8:789-792 (1997), the contents of which are incorporated herein by reference in their entirety.

In another preferred embodiment (V8), V comprises the following structure:

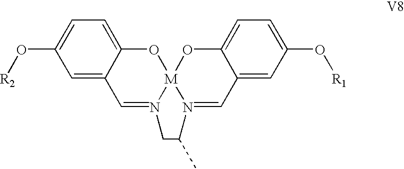

Wherein M is iron (II) or copper (II) and the dotted line is the site of linker attachment to RN; and wherein R₁ and R₂ are H or R₁ and R₂ are bioreversable masking groups for the p-hydroxy groups. In a preferred embodiment, R₁ and R₂ are acyl groups. Cleavage of the esters unmasks p-hydroxy groups, which can trigger the reaction of the complex with oxygen and targeted free radical formation.

Another general method to convert rn into a neoantigen is to use a drug E-T in which E is comprised of a mechanism based suicide inhibitor of the target rn. Mechanism based suicide inhibitors are a class of enzyme inhibitors that are converted by the catalytic activity of an enzyme into a product that irreversibly modifies and inactivates the enzyme.

Patients can be sensitized to the neoantigen either by immunization with the covalently modified target protein or with synthetic oligopeptides that correspond to the modified portion of the targeted proteins. The target protein rn can be modified in a defined manner and can generate defined and identifiable modified oligopeptide fragments upon intracellular proteolytic processing. The patient can also be immunized with these modified oligopeptide fragments. The advantage of this approach is that small chemically defined oligopeptides that correspond to the actual neoantigens, presented by host MHC molecules, can be employed for sensitization rather then complex proteins. The neoantigens generated can be characterized by employing standard labeling and biochemical techniques commonly used to identify the site of affinity labeling of enzymes.

Prostate Specific Antigen Targeted Neoantigens

Prostatic adenocarcinoma cells produce prostatic specific antigen (PSA), a serine protease, which is released into the tumor microenvironment. PSA is both a clinically useful marker for prostate cancer and an attractive target for prostate cancer therapies since the enzyme is expressed in large quantities by a high percentage of prostate cancers. Doxorubicin prodrugs, designed to be selectively activated by PSA, have been described. PSA is rapidly inactivated by alpha 2-macroglobulin and alpha 1-antichymotrypsin in the circulation. A variety of vaccination approaches against PSA have been developed and are in clinical trials.

PSA is a chymotrypsin-like serine protease with a preference for the cleavage of the tyrosine-serine bond in oligopeptides of the sequence Ser-Ser-Phe-Tyr-Ser. Other peptide sequences such as His-Ser-Ser-Lys-Leu-Gln-X are also substrates. The catalytic site of PSA bears striking similarity to chymotrypsin, human glandular kallikrein, and tonin. Serine proteases, as a family, are characterized by conserved features in the catalytic active site and are subject to irreversible inactivation by a variety of well-studied mechanism based suicide inhibitors. The very action of the enzyme on the inhibitor results in covalent modification of the enzyme and generates a neoantigen or neoantigen precursor. A neoantigen precursor yields a neoantigen upon cellular proteolytic processing. The following references relate to this subject matter: Coombs G. S., et al., “Substrate Specificity of Prostate-Specific Antigen (PSA),” Chem & Biol, 5(9):475-488 (1998); Villoutreix B. O., et al., “A Structural Model for the Prostate Disease Marker, Human Prostate-specific Antigen,” Protein Sci, 3:2033-2044 (1994); Vihinen, Mauno, “Modeling of Prostate Specific Antigen and human Glandular Kallikrein Structures,” Biochem Biophys Res Comm, 204(3):1251-1256 (1994); Denmeade S. R., et al., “Enzymatic Activation of a Doxorubicin-Peptide Prodrug by Prostate-Specific Antigen,” Cancer Res, 58:2537-2540 (1998); Christensson A., et al., “Enzymatic Activity of Prostate-Specific Antigen and its Reactions with Extracellular Serine Proteinase Inhibitors,” Eur J Biochem, 194(3):755-63 (1990); Zhang W. M., et al., “Characterization and Immunological Determination of the Complex Between Prostate-Specific Antigen and Alpha2-Macroglobulin,” Clin Chem, 44(12):2471-9 (1998); Meidenbauer N., et al., “Generation of PSA-reactive Effector Cells after Vaccination with a PSA-based Vaccine in Patients with Prostate Cancer,” Prostate, 43(2):88-100 (2000); Correale P., et al., “In Vitro Generation of Human Cytotoxic T Lymphocytes Specific for Peptides Derived from Prostate-Specific Antigen,” J Natl Cancer Inst, 89(4):293-300 (1997); Sanda M. G., et al., “Recombinant Vaccinia-PSA (PROSTVAC) can Induce a Prostate-Specific Immune Response in Androgen-Modulated Human Prostate Cancer,” Urology, 53(2):260-6 (1999); Slovin S. F.; Scher H. I., “Peptide and Carbohydrate Vaccines in Relapsed Prostate Cancer: Immunogenicity of Synthetic Vaccines in Man—Clinical Trials at Memorial Sloan-Kettering Cancer Center,” Semin Oncol, 26(4):448-54 (1999), the contents of which are incorporated herein by reference in their entirety.

In a preferred embodiment (Eneo1), the effector group E of the drug E-T comprises a mechanism-based inhibitor of PSA. Alpha-(aminoalkyl)phosphonate diphenyl esters irreversibly inactivates serine proteases by phosphonylating serine in the catalytic site. The following references relate to this subject matter: Oleksyszyn, Jozef; Powers, James C., “Irreversible Inhibition of Serine Proteases by Peptidyl Derivatives of α-Aminoalkylphosphonate Diphenyl Esters,” Biochem Biophys Res Comm, 161(1):143-149 (1989); Oleksyszyn, Jozef; Powers, James C., “Irreversible Inhibition of Serine Proteases by Peptide Derivatives of (α-Aminoalkyl)phosphonate Diphenyl Esters,” Biochem, 30:485-493 (1991); Oleksyszyn J., et al., “Novel Amidine-Containing Peptidyl Phosphonates as Irreversible Inhibitors for Blood Coagulation and Related Serine Proteases,” J Med Chem, 37:226-231 (1994), the contents of which are incorporated herein by reference in their entirety.

In a preferred embodiment (Eneo2), E is comprised of a compound given by the following structure:

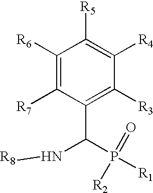

wherein either R₁ or R₂ is a good leaving group for nucleophilic substitution reactions at phosphorus; and wherein R₃-R₇ can be H, Cl, Br, F, I, a lower alkyl group, a lower alkoxy group, OH, or NO₂; and wherein R₈ is an oligopeptide or oligopeptide analog connected to the remainder of the drug.

In a preferred embodiment (Eneo3), E comprises the following structure:

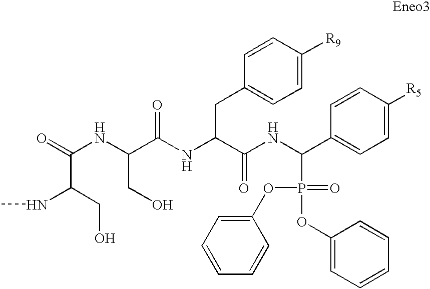

wherein R₅ and R₉ are H, or OH, and wherein the dashed line indicates the site of linker attachment to the remainder of the drug.

The inhibition of PSA, by a compound of the above structure, can give rise to a family of neoantigens derived from PSA in which the hydroxy group of the serine of the catalytic triad is phosphonylated. Patients can be sensitized to these neoantigens by immunization with PSA that has been modified by treatment with an inhibitor of related structure such as:

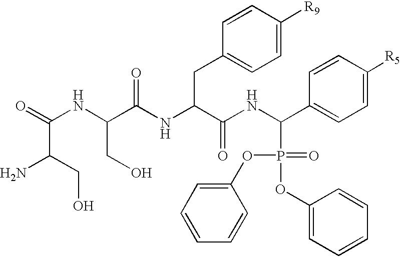

Alternatively, the pateint can be sensitized by immunization with oligopeptide fragments containing the region of the PSA protein that bears the phosphonylated serine residue. The primary amino acid sequence of PSA is known; therefore, the sequence of the neoantigen family is also known.

In another preferred embodiment E is a haloenol based mechansim based suicide inhibitor of PSA. Haolenol lactones are a class of irreversible serine protease inhibitors, which alkylate the enzyme. The following references relate to this subject matter: Baek D. J., et al., “Alternate Substrate Inhibitors of an alpha-Chymotrypsin: Enantioselective Interaction of Aryl-Substituted Enol Lactones,” Biochemistry, 29(18): 4305-11 (1990); Sofia M. J., et al., “Enol Lactone Inhibitors of Serine Proteases. The Effect of Regiochemistry on the Inactivation Behavior of Phenyl-Substituted (Halomethylene)Tetra- and -Dihydrofuranones and (Halomethylene) Tetrahydropyranones Toward Alpha-Chymotrypsin: Stable Acyl Enzyme Intermediate,” J Med Chem, 29(2):230-8 (1986); Raj R., et al., “Guanidinophenyl-Substituted Enol Lactones as Selective, Mechanism-Based Inhibitors of Trypsin-Like Serine Proteases,” J Med Chem, 35(22):4150-9 (1992); Baek D. J. and Katzenellenbogen J. A., “Halo Enol Lactone Inhibitors of Chymotrypsin: Burst Kinetics and Enantioselectivity of Inactivation,” Biochem Biophys Res Commun, 178(3):1335-42 (1991); Reed P. E., et al., “Proline-Valine Pseudo Peptide Enol Lactones. Effective and Selective Inhibitors of Chymotrypsin and Human Leukocyte Elastase,” J Biol Chem, 266(1):13-21 (1991); Daniels S. B. et al., “Halo Enol Lactones: Studies on the Mechanism of Inactivation of Alpha-Chymotrypsin,” Biochem, 25(6):1436-44 (1986); Rai R. and Katzenellenbogen J. A., “Effect of Conformational Mobility and Hydrogen-Bonding Interactions on the Selectivity of Some Guanidinoaryl-Substituted Mechanism-Based Inhibitors of Trypsin-like Serine Proteases,” J Med Chem, 35:4297-4305 (1992); Daniels S. B., et al., “Haloenol Lactones,” J Biol Chem, 258(24):15046-15053 (1983); Baek DJ, et al., “Alternate Substrate Inhibitors of Chymotrypsin: Enantioselective Interaction of Aryl-Substituted Enol Lactones,” Biochem, 29:4305-4311 (1990); Baek D J and Katzenellenbogen J. A., “Halo Enol Lactone Inhibitors of Chymotrypsin: bust Kinetics and Enantioselectivity of Inactivation,” Biochem Biophys Res Comm, 178(3):1335-1342 (1991), the contents of which are incorporated herein by reference in their entirety.

In a preferred embodiment (Eneo4), E is comprised of a compound given by the following structure:

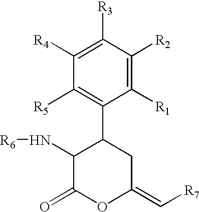

wherein R₁-R₅ can be H, Cl, Br, F, I, a lower alkyl group, a lower alkoxy group, OH, or NO₂; and wherein R₆ is an oligopeptide or oligopeptide analog connected to the remainder of the drug, or wherein R₆ is a linker connected to the remainder of the drug, and wherein R₇ is Cl, Br, F, I.

In a preferred embodiment (Eneo5), R₁, R₂, R₄, and R₅ are H, and R₃ is H or OH. In another preferred embodiment, E comprises the following structure:

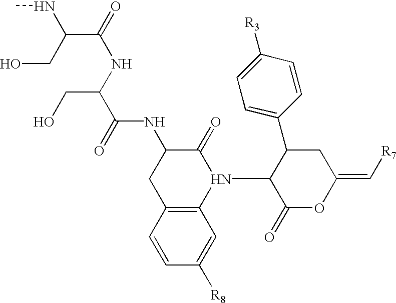

wherein R₃ and R₈ are H, or OH, and the dashed line indicates the site of linker attachment to the remainder of the drug.

Human Glandular Kallikrein 2 Targeted Neoantigens

Human glandular kallikrein 2 (HGK2) is a prostate specific serine protease that is closely related to PSA. HGK2 cleaves amide bonds adjacent to arginine residues. For example, H-D-Pro-Phe-Arg-p-nitroanilide is cleaved by HGK2. HGK2 is an excellent marker for prostate cancer that is over-expressed in essentially all prostate cancer. Increased invasiveness of prostate cancer is accompanied by increased expression of HGK2. HGK2, like PSA, is also expressed in a significant proportion of human breast cancers. The enzyme is rapidly inactivated by normal plasma protease inhibitors. Currently no methodology exists to exploit the tremendous potential of HGK2 as a target for the therapy of prostate and breast cancer. The following references relate to this subject matter: Heeb M. J., et al., “alpha2-Macroglobulin and C1-Inactivator are Plasma Inhibitors of Human Glandular Kallikrein,” Blood Cells Mol Dis, 24(4):412-419 (1998), Grauer L. S., et al., “Detection of Human Glandular Kallikrein, Hk2, as its Precursor Form and in Complex with Protease Inhibitors in Prostate Carcinoma Serum,” J Androl, 19(4):407-11 (1998), Kumar A., et al., “Expression of Human Glandular Kallikrein, hK2, in Mammalian Cells,” Cancer Res, 56(23):5397-402 (1996); Darson M. F., et al., “Human Glandular Kallikrein 2 (hk2) Expression in Prostatic Intraepithelial Neoplasia and Adenocarcinoma: A Novel Prostate Cancer Marker,” Urology, 49(6):857-62 (1997); Darson M. F., et al., “Human Glandular Kallikrein 2 Expression in Prostate Adenocarcinoma and Lymph Node Metastases,” Urology, 53(5):939-44 (1999); Mikolajczyk S. D., et al., “Human Glandular Kallikrein, hk2, Shows Arginine-Restricted Specificity and Forms Complexes with Plasma Protease Inhibitors,” Prostate, 34(1):44-50 (1998); Grauer L. S., et al., “Identification of Human Glandular Kallikrein hHk2 from LNCaP Cells,” J Androl, 17(4):353-9 (1996); McGarvey T., et al., “In Situ Hybridization Studies of Alpha 2-Macroglobulin Receptor and Receptor-Associated Protein in Human Prostate Carcinoma,” Prostate, 28(5):311-7 (1996); Saedi M. S., et al., “Over-expression of a Human Prostate-Specific Glandular Kallikrein, hk2, In E. Coli and Generation of Antibodies,” Mol Cell Endocrinol, 109(2):23741 (1995), the contents of which are incorporated herein by reference in their entirety.

In a preferred embodiment of the invention, E is a mechanism based suicide inhibitor for HGK2.

In a preferred embodiment (Eneo6), E comprises the following structure:

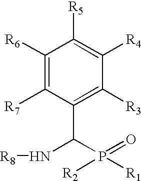

wherein either R₁ or R₂ is a good leaving group for nucleophilic substitution reactions at phosphorus; and wherein R₃, R₄, R₆, and R₇ can be H, Cl, Br, F, I, a lower alkyl group, a lower alkoxy group, OH, or NO₂; and wherein R₅ is an amidino group, a guanidino group, or a positively charged group, and R₈ is an oligopeptide or oligopeptide analog connected to the remainder of the drug.

In a preferred embodiment (Eneo7), E comprises the following structure:

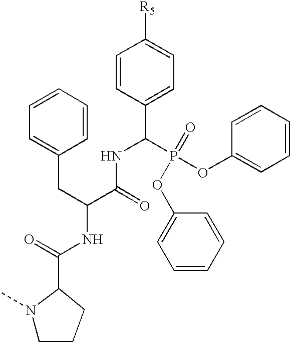

wherein R₅ is an amidino group, or a guanidino group.

In another preferred embodiment, E is a haloenol mechansim based suicide inhibitor of HGK2. Haolenol lactones are a class of irreversible serine protease inhibitors that alkylate the enzyme. In a preferred embodiment (Eneo8), E is comprised of a compound given by the following structure:

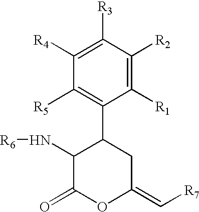

wherein R₁, R₂, R₄, and R₅ can be H, Cl, Br, F, I, a lower alkyl group, a lower alkoxy group, OH, or NO₂; and wherein R₃ is an amidino group, a guanidino group, or a positively charged group. R₇ is Cl, Br, F, I, and R₆ is an oligopeptide or oligopeptide analog connected to the remainder of the drug.

In a preferred embodiment designated as (Eneo9), R₁, R₂, R₄, and R₅ are H, and R₃ is an amidino, or guanidino group.

In a preferred embodiment (Eneo10), E comprises the following structure:

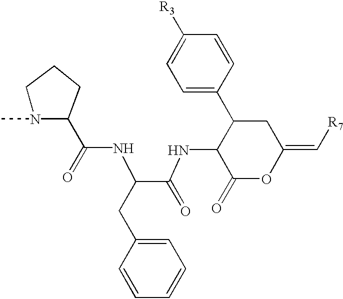

wherein R₃ is an amidino or a guanidino group and R₇ is Cl, Br, F, I, and the dotted line is the site of linker attachment to the remainder of the drug.

Patients can be sensitized to the neoantigens that arise from the modification of HGK2 by inhibitors using the same approaches as described for PSA derived neoantigens.

Plasmin Targeted Neoantigens

Most human malignancies are characterized by the elevated expression of urokinase and tissue plasminogen activator that results in the activation of plasminogen into plasmin. Tumor-associated plasmin can serve as an excellent tumor marker for neoantigen directed therapy. Plasmin is a serine protease with specificity for cleaving amide bonds adjacent to lysine and arginine. For example, benzyloxycarbonyl-D-IIe-Phe-Lys-p-nitroanilide is an excellent substrate for plasmin.

In a preferred embodiment of the invention, E is a mechanism based suicide inhibitor for plasmin.

In a preferred embodiment (Eneo11), E comprises the following structure:

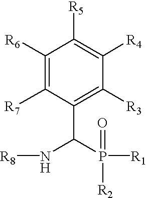

wherein either R₁ or R₂ is a good leaving group for nucleophilic substitution reactions at phosphorus; and wherein R₃, R₄, R₆, and R₇ can be H, Cl, Br, F, I,, a lower alkyl group, a lower alkoxy group, OH, or NO₂; and wherein R₅ is an amidino group, a guanidino group, or a positively charged group, and R₈ is an oligopeptide or oligopeptide analog connected to the remainder of the drug.

In a preferred embodiment (Eneo12), E comprises the following structure:

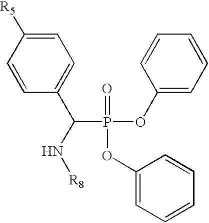

wherein R₅ is an amidino or guanidino group.

In another preferred embodiment (Eneo13), E comprises the following structure:

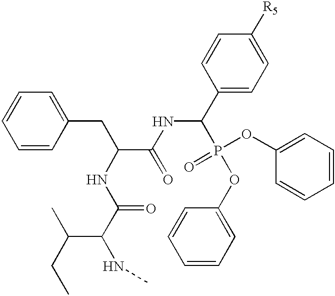

wherein R₅ is an amidino or guanidino group, and the dotted line is the site of linker attachment to the remainder of the drug.

In another preferred embodiment, E is a haloenol mechansim based suicide inhibitor of plasmin. In a preferred embodiment (Eneo14), E is comprised of a compound given by the following structure:

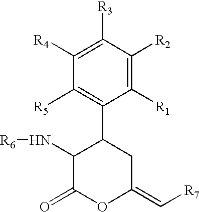

wherein R₁, R₂, R₄, and R₅ can be H, Cl, Br, F, I, a lower alkyl group, a lower alkoxy group, OH, or NO₂; and wherein R₃ is an amidino group, a guanidino group, or a positively charged group. R₇ is Cl, Br, F, I, and R₆ is an oligopeptide or oligopeptide analog connected to the remainder of the drug.

In a preferred embodiment (Eneo15), R₁, R₂, R₄, and R₅ are H, and R₃ is an amidino, or guanidino group.

In another preferred embodiment (Eneo16), E comprises the following structure:

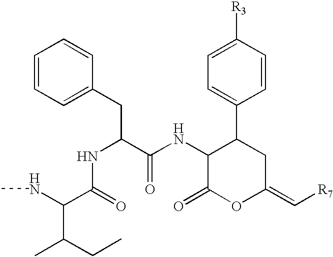

wherein R₃ is an amidino or a guanidino group and R₇ is Cl, Br, F, I, and the dotted line is the site of linker attachment to the remainder of the drug.

Urokinase Targeted Neoantigens

Urokinase is a serine protease, which is over-expressed by most human malignancies and functions to activate plaminogen into plasmin on the surface of tumor cells. Urokinase preferentially cleaves amide bonds adjacent to arginine and lysine residues.

In a preferred embodiment (Eneo17), E is a mechanism based suicide inhibitor for urokinase.

In a preferred embodiment (Eneo18), E comprises the following structure:

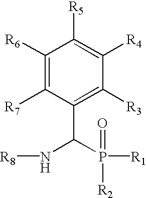

wherein either R₁ or R₂ is a good leaving group for nucleophilic substitution reactions at phosphorus; and wherein R₃, R4, R₆, and R₇ can be H, Cl, Br, F, I, a lower alkyl group, a lower alkoxy group, OH, or NO₂; and wherein R₅ is an amidino group, a guanidino group, or a positively charged group and R₈ is an oligopeptide or oligopeptide analog connected to the remainder of the drug.

In a preferred embodiment (Eneo18), E comprises the following structure:

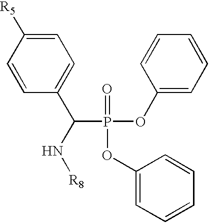

wherein R₅ is an amidino or guanidino group.

In another preferred embodiment (Eneo19), E comprises the following structure:

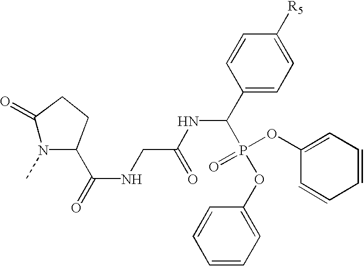

wherein R₅ is an amidino or guanidino group, and the dotted line is the site of linker attachment to the remainder of the drug.

In another preferred embodiment, E is a haloenol mechansim based suicide inhibitor of urokinase. In a preferred embodiment (Eneo20) E is comprised of a compound given by the following structure:

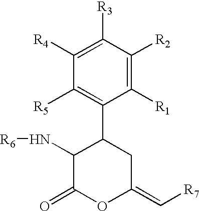

wherein R₁, R₂, R₄, and R₅ can be H, Cl, Br, F, I, a lower alkyl group, a lower alkoxy group, OH, or NO₂; and wherein R₃ is an amidino group, a guanidino group, or a positively charged group. R₇ is Cl, Br, F, I, and R₆ is an oligopeptide or oligopeptide analog connected to the remainder of the drug.

In a preferred embodiment, R₁, R₂, R₄, and R₅ are H, and R₃ is an amidino, or guanidino group.

In another preferred embodiment (Eneo21), E comprises the following structure:

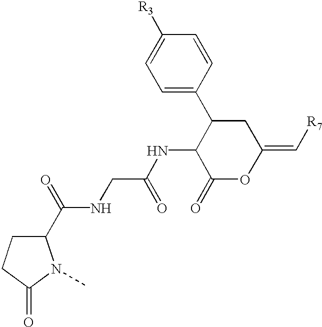

Wherein R₃ is an amidino or a guanidino group and R₇ is Cl, Br, F, I, and the dotted line is the site of linker attachment to the remainder of the drug.

Matriptase Targeted Neoantigens

Matriptase is a typical serine protease with gelatinase activity that is expressed on the surface of breast cancer cells. The enzyme like trypsin and urokinase cleaves preferentially amide bonds adjacent to arginine or lysine residues. The following references relate to this subject matter: Lin C. Y., et al.,“Characterization of a Novel, Membrane-Bound, 80-kDa Matrix-Degrading Protease from Human Breast Cancer Cells. Monoclonal Antibody Production, Isolation, and Localization,” J Biol Chem, 272(14):9147-52 (1997); Lin C. Y., et al., “Molecular Cloning of cDNA for Matriptase, a Matrix-Degrading Serine Protease with Trypsin-Like Activity,” J Biol Chem, 274(26):18231-6 (1999); Lin C. Y., et al., “Purification and Characterization of a Complex Containing Matriptase and a Kunitz-Type Serine Protease Inhibitor from Human Milk,” J Biol Chem, 274(26):18237-42 (1999), the contents of which are incorporated herein by reference in their entirety.

In a preferred embodiment, E is a mechanism based suicide inhibitor for matriptase. The same structures described above for urokinase can be employed to generate neoantigens for matriptase.

Fibroblast Activation Protein Targeted Neoantigens

Fibroblast Activation Protein (FAP) is a serine protease with gelatinase and prolyl oligopeptidase activity. FAP is expressed on the surface of tumor-associated fibroblasts in the vast majority of human malignancies including: breast, colon, lung, ovarian, and pancreatic cancer. In addition, the enzyme is present on the surface of human malignancies of mesenchymal origin such as fibrosarcomas and osteogenic sarcomas. FAP is also expressed on fibroblasts during wound healing. The potential of FAP as an almost universal tumor target has been appreciated for many years but remains to be exploited. These considerations make FAP an excellent tumor-associated target for neoantigen directed immunotherapy. As discussed previously, the induction of an intense immune reaction in the tumor stroma can exert pronounced antitumor activity by nonspecific mechanisms. For sarcomas and other FAP+ tumor cell types, the drug can be targeted to the tumor cells. For tumors in which the stromal cells are FAP positive and the tumor cells are FAP negative, the drug can be targeted to other features of tumor-associated fibroblasts by the targeting ligands of ET. Numerous other targets are known to be enriched on tumor-associated fibroblasts including a variety of matrix metalloproteinases and collagenases. The following references relate to this subject matter: Niedermeyer J., et al., “Mouse Fibroblast-Activation Protein—Conserved Fap Gene Organization and Biochemical Function as a Serine Protease,” Eur J Biochem, 254(3):650-4 (1998); Park J. E., et al., “Fibroblast Activation Protein, a Dual Specificity Serine Protease Expressed in Reactive Human Tumor Stromal Fibroblasts,” J Biol Chem, 274(51):36505-36512 (1999); Mueller S. C., et al., “A Novel Protease-docking Function of Integrin at Invadopodia,” J Biol Chem, 274(35):24947-24952 (1999); Scanlan M. J., et al., “Molecular Cloning of Fibroblast Activation Protein α, a Member of the Serine Protease Family Selectively Expressed in Stromal Fibroblasts of Epithelial Cancers,” Proc Natl Acad Sci USA, 91:5657-5661 (1994); Goldstein L. A., et al., “Molecular Cloning of Seprase: a Serine Integral Membrane Protease from Human Melanoma,” Biochim Biophys Acta, 1361(1):11-9 (1997); Rettig W. J., et al., “Fibroblast Activation Protein: Purification, Epitope Mapping and Induction by Growth Factors,” Int J Cancer, 58(3):385-92 (1994); Levy M. T., et al., “Fibroblast Activation Protein: a Cell Surface Dipeptidyl Peptidase and Gelatinase Expressed by Stellate Cells at the Tissue Remodelling Interface in Human Cirrhosis,” Heptagoloty, 29(6):1768-78 (1999); Rettig W. J., et al., “Regulation and Heteromeric Structure of the Fibroblast Activation Protein in Normal and Transformed Cells of Mesenchymal and Neuroectodermal Origin,” Cancer Res, 50(14):3327-35 (1993); Niedermeyer J., et al., “Targeted Disruption of Mouse Fibroblast Activation Protein,” Molec Cell Biol, 20(3):1089-1094 (2000); Welt S., et al., “Antibody Targeting in Metastatic Colon Cancer: a Phase I Study of Monoclonal Antibody F19 Against a Cell-surface Protein of Reactive Tumor Stromal Fibroblasts,” J Clin Oncol, 12(6):1193-203 (1994); Garin-Chesa P., et al., “Cell Surface Glycoprotein of Reactive Stromal Fibroblasts as a Potential Antibody Target in Human Epithelial Cancers,” Immunology, 87:7235-7239 (1990), the contents of which are incorporated herein by reference in their entirety.

In a preferred embodiment, E is a mechanism based suicide inhibitor for FAP.

In a preferred embodiment (Eneo22), E comprises the following structure:

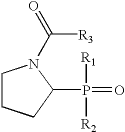

wherein either R₁ or R₂ is a good leaving group for nucleophilic substitution reactions at phosphorus. R₃ is an oligopeptide, oligopeptide analog, or a linker connected to the remainder of the drug.

In a preferred embodiment (Eneo23), E comprises the following structure:

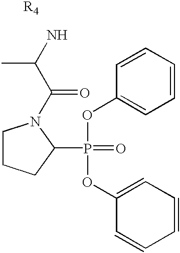

wherein R₄ is the site of linker attachment to the remainder of ET.

In another preferred embodiment (Eneo25), E comprises the following structure:

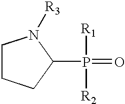

wherein either R₁ or R₂ is a good leaving group for nucleophilic substitution reactions at phosphorus. R₃ is an amino acid, coupled via its carboxylic group, and wherein either R₁ or R₂ has a site to which a linker is attached to the remainder of the drug.

In a preferred embodiment (Eneo26), E comprises the following structure:

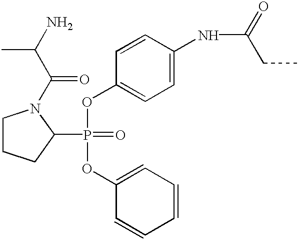

Wherein the dotted line is the site of linker attachment to the remainder of the drug.

In another preferred embodiment, E is a haloenol mechansim based suicide inhibitor of FAP. In a preferred embodiment (Eneo27), E is comprised of a compound given by the following structure:

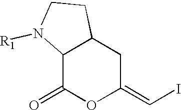

wherein R₁ is an oligopeptide, oligopeptide analog, or a linker connected to the remainder of the drug.

Seprase Targeted Neoantigens

Seprase is a serine protease that is very similar if not identical to FAP. The enzyme is over-expressed on the surface of malignant melanoma and breast cancer cells. The following references relate to this subject matter: Levy M. T., et al., “Fibroblast Activation Protein: a Cell Surface Dipeptidyl Peptidase and Gelatinase Expressed by Stellate Cells at the Tissue Remodelling Interface in Human Cirrhosis,” Hepatology, 29(6):1768-78 (1999); Mueller S. C., et al., “A Novel Protease-docking Function of Integrin at Invadopodia,” J Biol Chem, 35:24947-24952 (1999); Goldstein L. A., et al., “Molecular Cloning of Seprase: A Serine Integral Membrane Protease from Human Melanoma,” Biochem Biophys Acta, 1361(1):11-9 (1997); Kelly T., “Evaluation of Seprase Activity,” Clin Exp Metastasis, 17(1):57-62 (1999); Goldstein L. A.; Chen W. T., “Identification of an Alternatively Spliced Seprase mRNA that Encodes a Novel Intracellular Isoform,” J Biol Chem, 275(4):2554-2559 (2000); Pineiro-Sanchez M. L., et al., “Identification of the 170-kDa Melanoma Membrane-Bound Gelatinase (Seprase) as a Serine Integral Membrane Protease,” J Biol Chem, 272(12):7595-601 (1997); Mueller S. C., et al., “A Novel Protease-docking Function of Integrin at Invadopodia,” J Biol Chem, 274(35):24947-24952 (1999); Monsky W. L., et al., “A Potential Marker Protease of Invasiveness, Seprase, is Localized on Invadopodia of Human Malignant Melanoma Cells,” Cancer Res, 54(21):5702-10 (1994); Scanlan M. J., et al., “Molecular Cloning of Fibroblast Activation Protein Alpha, a Member of the Serine Protease Family Selectively Expressed in Stromal Fibroblasts Of Epithelial Cancers,” Proc Natl Acad Sci USA, 91(12):5657-61 (1994); Goldstein L. A., et al., “Molecular Cloning of Seprase: A Serine Integral Membrane Protease From Human Melanoma,” Biochim Biophys Acta, 1361(1):11-9 (1997); Kelly T., et al., “Seprase, a Membrane-Bound Protease, is Over-expressed by Invasive Ductal Carcinoma Cells of Human Breast Cancers,” Mod Pathol, 11 (9):855-63 (1998); Niedermeyer J., et al., “Mouse Fibroblast Activation Protein: Molecular Cloning, Alternative Splicing and Expression in the Reactive Stroma of Epithelial Cancers,” Int J cancer, 71(3):383-9 (1997), the contents of which are incorporated herein by reference in their entirety.

In a preferred embodiment, E is a mechanism based suicide inhibitor for seprase. Structures described above for FAP can be used to generate neoantigens to seprase.

Fatty Acid Synthetase Targeted Neoantigens.

Fatty acid synthetase (FAS) is an enzyme, which catalyzes the synthesis of long chain fatty acids. The enzyme is over-expressed in breast cancer, colon cancer, ovarian, endometrial and prostate cancer. Inhibitors of FAS have been described as potential anti-cancer drugs, which trigger aptoptosis. Cerulenin is a mechanism based suicide inhibitor for FAS. A critical cysteine in the active site of the enzyme is alkiated by cerulenin. This modification generates a neoantigen precursor, which can be used to trigger an immune response. The following references relate to this subject matter: Funabashi H., et al., “Binding Site of Cerulenin in Fatty Acid Synthetase,” J Biochem, 105:751-755 (1989); Moche M., et al., “Structure of the Complex between the Antibiotic Cerulenin and Its Target, β-Ketoacyl-Acyl Carrier Protein Synthase,” J Biological Chem, 274(10):6031-6034 (1999); Kuhajda F. P., et al., “Synthesis and Antitumor Activity of an Inhibitor of Fatty Acid Synthase,” Proc Natl Acad Sci USA, 97(7):3450-3454 (2000); Pizer E. S., et al., “Pharmacological Inhibitors of Mammalian Fatty Acid Synthase Suppress DNA Replication and Induce Apoptosis in Tumor Cell Lines,” Cancer Res, 58(20):4611-5 (1998); Pizer E. S., et al., “Malonyl-coenzyme-A is a Potential Mediator of Cytotoxicity Induced by Fatty-Acid Synthase Inhibition in Human Breast Cancer Cells and Xenografts,” Cancer Res, 60(2):213-8 (2000); Gansler T. S., et al., “Increased Expression of Fatty Acid Synthase (OA-519) in Ovarian Neoplasms Predicts Shorter Survival,” Hum Pathol, 28(6):686-92 (1997); Visca P., et al., “Immunohistochemical Expression of Fatty Acid Synthase, Apoptotic-Regulating Genes, Proliferating Factors, and Ras Protein Product in Colorectal Adenomas, Carcinomas, and Adjacent Nonneoplastic Mucosa,” Clin Cancer Res, 5(12):4111-8 (1999); Kuhajda F. P., “Fatty-Acid Synthase and Human Cancer: New Perspectives on its Role in Tumor Biology,” Nutrition, 16(3):202-208 (2000); Krontiras H., et al., “Fatty Acid Synthase Expression is Increased in Neoplastic Lesions of the Oral Tongue,” Head Neck, 21(4):325-9 (1999); Nakamura I., et al., “Fatty Acid Synthase Expression in Japanese Breast Carcinoma Patients,” Int J Mol Med, 4(4):381-7 (1999); Pizer E. S., et al., “Fatty Acid Synthase Expression in Endometrial Carcinoma: Correlation with Cell Proliferation and Hormone Receptors,” Cancer, 83(3):528-37 (1998); Alo P. L., et al., “Fatty Acid Synthase (FAS) Predictive Strength in Poorly Differentiated Early Breast Carcinomas,” Tumori, 85(1):35-40 (1999); Milgraum L. Z., et al., “Enzymes of the Fatty Acid Synthesis Pathway are Highly Expressed in In Situ Breast Carcinoma,” Clin Cancer Res, 3(11):2115-20 (1997); Rashid A., et al., “Elevated Expression of Fatty Acid Synthase and Fatty Acid Synthetic Activity in Colorectal Neoplasia,” Am J Pathol, 150(1):201-8 (1997); Jayakumar A., et al., “Human Fatty Acid Synthase: Properties and Molecular Cloning,” Proc Nati Acad Sci USA, 92(19):8695-9 (1995); Hennigar R. A., et al., “Characterization of Fatty Acid Synthase in Cell Lines Derived from Experimental Mammary Tumors,” Biochim Biophys Acta, 1392(1):85-100 (1998); Swinnen J. V., et al., “Androgens Stimulate Fatty Acid Synthase in the Human Prostate Cancer Cell Line LNCaP,” Cancer Res, 57(6):1086-90 (1997); Kuhajda F. P., et al., “Fatty Acid Synthesis: A Potential Selective Target for Antineoplastic Therapy,” Proc Natl Acad Sci USA, 91(14):6379-83 (1994); Kusakabe T., et al., “Fatty Acid Synthase is Expressed Mainly in Adult Hormone-sensitive Cells or Cells with High Lipid Metabolism and in Proliferating Fetal Cells,” J Histochem Cytochem, 48:613-622 (2000), the contents of which are incorporated herein by reference in their entirety.

In a preferred embodiment, E is a mechanism based enzyme inhibitor of FAS.

In a preferred embodiment (Eneo28) E is comprised of cerulenin.

In a preferred embodiment (Eneo29), E comprises the following structure:

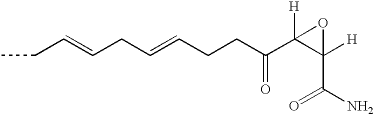

wherein the site of linker attachment to the rest of the drug is indicated by the dotted line.

The interaction of FAS and the above inhibitor can generate a neoantigen derived from FAS in which a cysteine of the enzyme is modified as shown below:

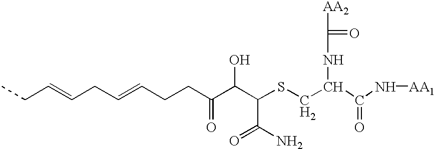

wherein AA1 and AA2 represent the amino acids adjacent to the modified cysteine residue. As in previous examples patients can be sensitized to the neoantigen by immunization with either appropriately modified FAS or by oligopeptides that correspond to the modified portion of the protein.

Steroid Sulfatase Targeted Neoantigens

Steroid sulfatase catalyzes the conversion of dehydroepiandrosterone sulfate and estrone sulfate into the unconjugated steroids. Steriod sulfatases are expressed in a variety of steriod dependent malignancies including breast cancer, ovarian, and endometrial cancer. Steroid sulfatase expression is an independent risk factor for tumor recurrence in breast cancer patients. A variety of inhibitors to steroid sulfatase have been developed as potential therapies to suppress estrogen production and estrogen dependent malignancies. A variety of steroidal and nonsteroidal sulfamates have been described which are mechanism based suicide inhibitors of steroid sulfatase. The enzyme is covalently modified by sulfamoylation.

In a preferred embodiment, E is a mechanism based suicide inhibitor for steroid sulfatase. In a preferred embodiment, E is a sulfamate based suicide inhibitor of steroid sulfatase.

In a preferred embodiment (Eneo30), E is the following structure:

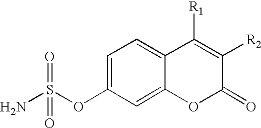

wherein R₁ and R₂ is a lower alkyl group, H, or a phenyl group; and wherein either R₁ or R₂ has a site for linker attachment to the remainder of the drug.

The neoantigen that results from the interaction of the inhibitor and steroid sulfatase can be a sulfamoylated enzyme. The pateint can be immunized either with this modified enzyme or with the corresponding sulfamolyated oligopeptide.

Epidermal Growth Factor Receptor Targeted Neoantigens

Epidermal growth factor receptors (EGFR) are membrane associated tyrosine kinases that are over-expressed in a large number of malignancies including: breast, prostate, ovarian, lung, gastric, and bladder. Aberrant activation of the tyrosine kinase activity results in neoplastic transformation. Accordingly, EGFR has attracted great attention as a target for anti-cancer therapy. Herceptin is a monoclonal antibody in clinical use for the treatment of breast cancer which binds to a member of the epidermal growth family (HER2) present on breast cancer cells. In patients with chemotherapy resistant metastatic HER2+ breast cancer treated with herceptin an objective response rate of 15% was observed. In hopes of improving therapy targeted to EGFR numerous inhibitors to EGFR have been developed. Unfortunately, inhibitors to EGFR tyrosine kinase are cytostatic rather than cytotoxic. The adenosine triphosphate binding site of EGFR has a reactive cysteine residue that is readily alkylated by a number of highly potent, selective irreversible inhibitors to EGFR. This covalent modification of the EGFR generates a neoantigen which can be exploited to target the immune system against EGFR+ cancers resulting in tumor cell death rather than just growth suppression. The following references relate to this subject matter: Cobleigh M. A., et al., “Multinational Study of the Efficacy and Safety of Humanized Anti-HER2 Monoclonal Antibody in Women who have HER2-Overexpressing Metastatic Breast Cancer that has Progressed after Chemotherapy for Metastatic Disease,” J Clin Oncology, 17(9):2639-2648 (1999); Discafani C. M., et al., “Irreversible Inhibition of Epidermal Growth Factor Receptor Tyrosine Kinase with In Vivo Activity by N-[4-[(3-Bromophenyl)amino]-6-quinazolinyl]-2-butynamide (CL-387,785),” Biochem Pharm, 57:917-925 (1999); Fry D. W., et al., “Specific, Irreversible Inactivation of the Epidermal Growth Factor Receptor and erbB2, by a New Class of Tyrosine Kinase Inhibitor,” Proc Natl Acad Sci USA, 95:12022-12027 (1998); Smaill J. B., et al., “Tyrosine Kinase Inhibitors. 4-(Phenylamino)quinazoline and 4-(Phenylamino)pyrido[d]pyrimidine Acrylamides as Irreversible Inhibitors of the ATP Binding Site of the Epidermal Growth Factor Receptor,” J Med Chem, 42:1803-1815 (1999); Rewcastle G. W., et al., “Tyrosine Kinase Inhibitors. 10. Isomeric 4-[(3-Bromophenyl)amino]pyrido[d]-pyrimidines are Potent ATP Binding Site Inhibitors of the Tyrosine Kinase Function of the Epidermal Growth Factor Receptor,” J Med Chem, 39:1823-1835 (1996); Rewcastle G. W., et al., “Tyrosine Kinase Inhibitors. 14. Structure-Activity Relationships for Methyl-amino-Substituted Derivatives of 4-[(3-Bromophenyl amino]-6-(methylamino)-pyrido[3,4-d]pyrimidine (PD 158780), a Potent and Specific Inhibitor of the Tyrosine Kinase Activity of Receptors for the EGF Family of Growth Factors,” J Med Chem, 41:742-751 (1998); Bridges A. J., et al., “Tyrosine Kinase Inhibitors. 8. An Unusually Steep Structure-activity Relationship for Analogues of 4-(3-Bromoanilino)-6,7-dimethoxyquinazoline (PD 153035), a Potent Inhibitor of the Epidermal Growth Factor Receptor,” J Med Chem, 39:267-276 (1996); Thompson, A. M., et al., “Tyrosine Kinase Inhibitors. 13. Structure-Activity Relationships for Soluble 7-Substituted 4-[(3-Bromophenyl)amino]pyrido[4,3-d]pyrimidines Designed as Inhibitors of the Tyrosine Kinase Activity of the Epidermal Growth Factor Receptor,” J Med Chem, 40:3915-3925 (1997); Rewcastle G. W., et al., “Tyrosine Kinase Inhibitors. 9. Synthesis and Evaluation of Fused Tricyclic Quinazoline Analogues as ATP Site Inhibitors of the Tyrosine Kinase Activity of the Epidermal Growth Factor Receptor,” J Med Chem, 39:918-928 (1996); Rewcastle G. W., et al., “Tyrosine Kinase Inhibitors. 12. Synthesis and Structure-Activity Relationships for 6-Substituted 4-(Phenylamino)pyrimido[5,4-d]pyrimidines Designed as Inhibitors of the Epidermal Growth Factor Receptor,” J Med Chem, 40:1820-1826 (1997); Smaill J. B., et al., “Tyrosine Kinase Inhibitors. 17. Irreversible Inhibitors of the Epidermal Growth Factor Receptor: 4-(Phenylamino)quinazoline- and 4-(Phenylamino)pyrido[3,2-d]pyrimidine-6-acrylamides Bearing Additional Solubilizing Functions,” J Med Chem, 43:1380-1397 (2000), the contents of which are incorporated herein by reference in their entirety.

In a preferred embodiment, E is an irreversible inhibitor to EGFR, which covalently modifies the protein generating a neoantigen.

In preferred embodiments (Eneo31 to Eneo42), E comprises the following structures:

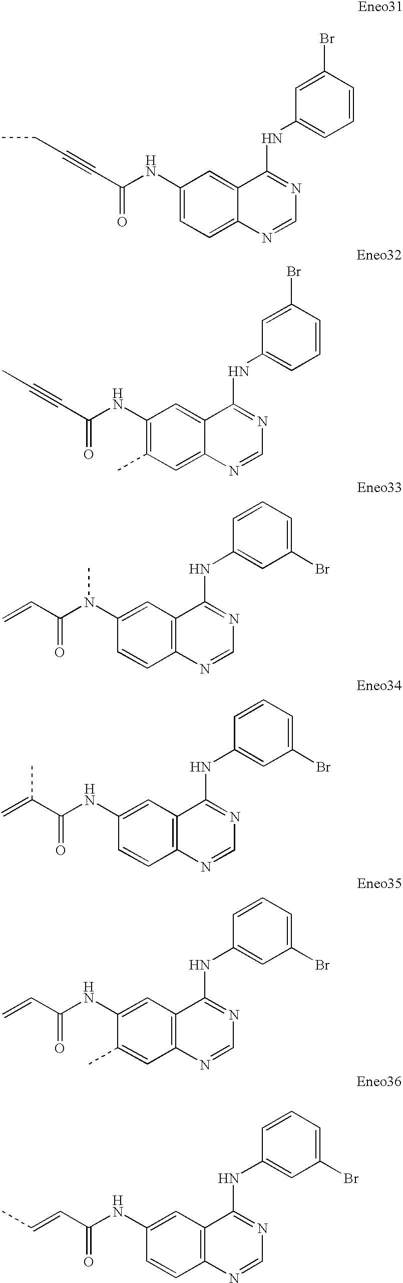

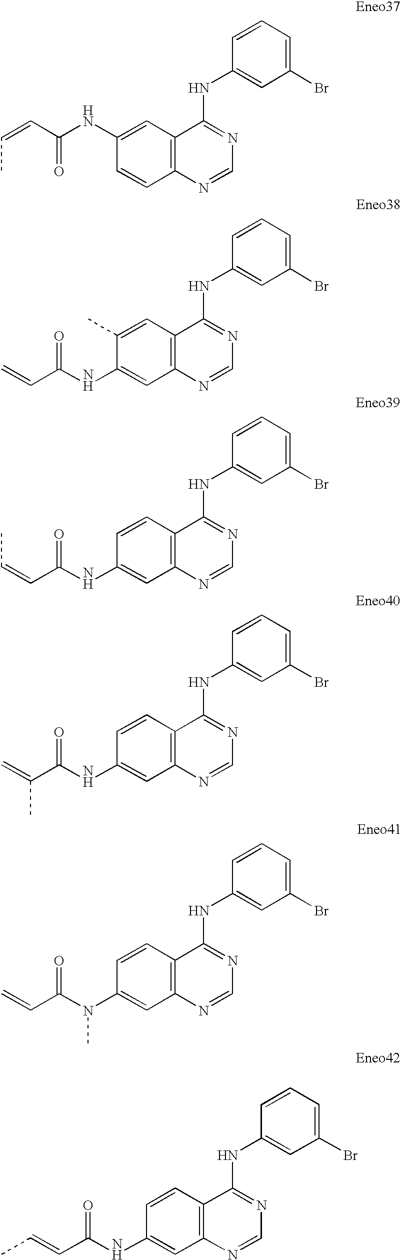

wherein the dotted line is the site of linker attachment to the remainder of the drug.

The neoantigens derived from the interaction of these inhibitors with EGFR can correspond to peptide sequences of the enzyme in which the thiol of cysteine 773 undergoes addition to the triple bond or the acrylamide double bond. Patients can be sensitized to these neoantigens either by immunization with the inhibited enzyme or by immunization with the corresponding modified oligopeptides neoantigens.

Phospatidylinositol 3-kinase Targeted Neoantigens

Phospatidylinositol 3-kinase (PIK3) is over-expressed in numerous malignancies including ovarian, breast, prostate, and lung cancer. The enzyme plays a key role in growth factor signal transduction. Over-expression results in neoplastic transformation. The PIK3CA oncogene, which is expressed in 40% of cases of ovarian cancer, encodes catalytic subunit of phosphatidylinositol 3-kinase. Accordingly, PIK3 is an attractive target for cancer therapy. A large number of inhibitors to the enzyme have been prepared as potential anti-cancer drugs. However, PIK3 inhibitors to date have exhibited toxicity and poor therapeutic index against tumors.

Wortmannin and related analogs are potent irreversible inhibitors of PIK3. The inhibitor covalently modifies the enzyme. The following references relate to this subject matter: Creemer L. C., et al., “Synthesis and in Vitro Evaluation of New Wortmannin Esters: Potent Inhibitors of Phosphatidylinositol 3-Kinase,” J Med Chem, 39:5021-5024 (1996); Powis G., et al., “Wortmannin, a Potent and Selective Inhibitor of Phosphatidylinositol-3-kinase,” Cancer Res, 54:2419-2423 (1994); Norman B. H., et al., “Studies on the Mechanism of Phosphatidylinositol 3-Kinase Inhibition by Wortmannin and Related Analogs,” J Med Chem, 39:1106-1111 (1996); Qiao L., et al., “3-Deoxy-D-myo-inositol 1-Phosphate, 1-Phosphonate, and Ether Lipid Analogues as Inhibitors of Phosphatidylinositol-3-kinase Signaling and Cancer Cell Growth,” J Med Chem, 41(18):3303-3306 (1998); Vlahos C. J., et al., “A Specific Inhibitor of Phosphatidylinositol 3-Kinase, 2-(4-Morpholinyl))-8-phenyl-4H-1 benzopyran-4-one (LY294002),” J Biol Chem, 269(7):5241-5248 (1994); Stefka Stoyanova et al, “Lipid Kinase and Protein Kinase Activities of G-Protein-Coupled Phosphoinositide 3-Kinase: Structure-Activity Analysis And Interactions with Wortmannin,” Biochem J, 324, 489-495 (1997); Wymann M. P., et al “Wortmannin Inactivates Phosphoinositide 3-Kinase by Covalent Modification of Lys-802, a Residue Involved in the Phosphate Transfer Reaction,” Mol Cell Biol, (4):1722-33 (1996), the contents of which are incorporated herein by reference in their entirety.

In a preferred embodiment, E is an irreversible inhibitor of PIK3.

In a preferred embodiment (Eneo43), E comprises the following structure:

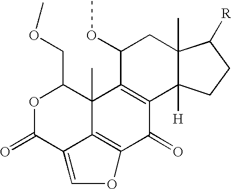

wherein the dotted line is the site of linker attachment to the remainder of ET and R is O, or OH.

Mitogen Activated Protein Kinase Kinase Targeted Neoantigens

Mitogen Activated Protein Kinase Kinase (MEK) plays a key role in growth factor signal transduction. Constitutive over-expression is oncogenic. Hyperactivity of the MEK-MAPK pathway is involved in numerous malignancies. Accordingly, MEK has attracted significant attention as a target for anti-cancer drugs. Inhibitors to MEK are cytostatic rather than cytotoxic and suppress tumor growth rather than killing tumors. Resorcylic acid lactones are extremely potent irreversible inhibitors of MEK. It is likely that the unsaturated alpha beta ketone alkylates a nucleophile in the active site of the enzyme. The covalently modified inhibited MEK can serve as a neoantigen for use in targeted immunotherapy. The following references relate to this subject matter: Zheng C. F; Guan K. L., “Cloning and Characterization of Two Distinct Human Extracellular Signal-regulated Kinase Activator Kinases, MEK1 and MEK2,” J Biol Chem, 268(15):11435-9 (1993); Salh B., et al., “Investigation of the Mek-MAP Kinase-Rsk Pathway in Human Breast Cancer,” Anti-cancer Res, 19(1B):731-40 (1999); Dudley D. T., et al., “A Synthetic Inhibitor of the Mitogen-Activated Protein Kinase Cascade,” Proc Nati Acad Sci USA, 92:7686-7689 (1995); Sebolt-Leopold J. S., et al., “Blockade of the MAP Kinase Pathway Suppresses Growth of Colon Tumors In Vivo,” Nature Med, 5(7):810-816 (1999); Zhao A., et al., “Resorcylic Acid Lactones: Naturally Occurring Potent and Selective Inhibitors of MEK,” J Antibiotics, 52(12):1086-1094 (1999); Hoshino R., et al., “Constitutive Activation of the 41-143-kDa Mitogen-activated Protein Kinase Signaling Pathway in Human Tumors,” Oncogene, 18:813-822 (1999); Duesbery N. S., et al., “MEK Wars, a New Front in the Battle Against Cancer,” Nature Med, 5(7):736-737 (1999), the contents of which are incorporated herein by reference in their entirety.

In a preferred embodiment, E is an irreversible inhibitor of MEK. In a preferred embodiment (Eneo44), E comprises the following structure:

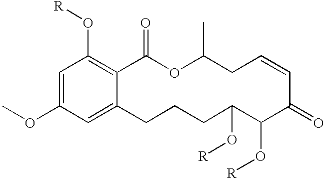

wherein R is H or the site of attachment to the remainder of the targeted drug by a trigger.

Glutathione -Transferase Targeted Neoantigens

Glutathione S-Transferases (GST) are over-expressed by a variety of malignancies including ovarian, breast, renal, colon and lung cancer. GST can be massively over-expressed in chemotherapy resistant tumor cells. Haloenol lactones are mechanism based enzyme inhibitors of Pi type GST. The haloenol lactones covalently modify GST. In the process, a neoantigen is generated which can be exploited for targeted immunotherapy. The following references relate to this subject matter: Mitchell A. E., et al., “Structural and Functional Consequences of Haloenol Lactone Inactivation of Murine and Human Glutathione S-Transferase,” Biochemistry, 27:6752-6759 (1998); Zheng J., et al., “Haloenol Lactone is a New Isozyme-selective and Active Site-directed Inactivator of Glutathione S-Transferase,” J Biol Chem, 271(34):20421-20425 (1996), the contents of which are incorporated herein by reference in their entirety.

In a preferred embodiment, E is a mechanism based suicide inhibitor of GST. In a preferred embodiment, E is a haloenol lactone mechanism based inhibitor of GST. In a preferred embodiment (Eneo45), E comprises the following structure:

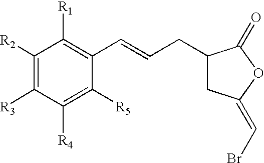

wherein R₁-R₅ can be H, Cl, Br, F, I, a lower alkyl group, a lower alkoxy group, OH or NO₂, an amino group, a cyano group, a carboxylate group, a phosphate, a phosphonate group, a sulfonate group, an ester group, or an amide group, and wherein either R₁, R₂, R₃, R₄, or R₅ has a site of attachment to the remainder of the target drug.

The neoantigen for immunization purposes can be prepared by treating the enzyme with an inhibitor based on the above structure.

Thymidylate Synthase Targeted Neoantigens

Thymidylate synthase (TS) catalyzes the synthesis of conversion of deoxyuridine 5′-monophosphate into thymidine 5′-monophosphate. TS provides the sole de novo source of thymidylate, a key precursor for DNA synthesis. TS is over-expressed in a variety of malignancies including colorectal, and breast. TS has long been recognized as an important target in cancer therapy and is the basis of a number of anti-cancer drugs currently in clinical use. Clinical short comings of current TS inhibitors include toxicity, and the development of tumor resistance by over-expression of TS, thymidine kinase and the nucleoside transporter proteins.

A number of mechanism based suicide inhibitors of thymidylate synthase that covalently modify the enzyme are known. For example, 5-(3-fluoropropyn-1-yl)-2′-deoxyuridine 5′ phosphate is a potent covalent inhibitor of TS. The interaction of TS and such an inhibitor can generate neoantigens, which can be exploited in targeted immunotherapy. The following references relate to this subject matter: Aschele C., et al., “Immunohistochemical Quantitation of Thymidylate Synthase Expression in Colorectal Cancer Metastases Predicts for Clinical Outcome to Fluorouracil-Based Chemotherapy,” J Clin Oncology, 17(6):1760-1770 (1999); Lobo A. P., et al., “Mode of Action of Site-Directed Irreversible Folate Analogue Inhibitors of Thymidylate Synthase,” Biochem, 37:4535-4542 (1998); Kalman T. I., et al., “Mechanism-Based Inactivation of Thymidylate Synthase by 5-(3-Fluoropropyn-1-yl)-2′-deoxyuridine 5′-Phosphate,” Biorg Med Chem Let, 10:391 -394 (2000); Bastian G., et al., “Inhibition of Thymidylate Synthetase by 5-Alkynyl-2′-deoxyuridylates,” J Med Chem, 24:1385-1388 (1981); Montgomery J. A., et al., “Phosphonate Analogue of 2′-deoxy-5-fluorouridylic Acid,” J Med Chem, 22(1):109-11 (1979), the contents of which are incorporated herein by reference in their entirety.

In a preferred embodiment, E is a mechanism based suicide inhibitor of TS. In a preferred embodiment (Eneo46), E comprises the following structure:

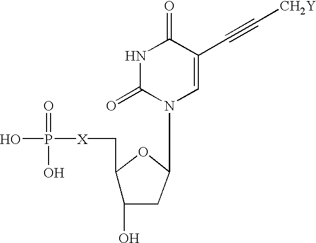

wherein X is O, CH₂, CHF, and CF₂, and Y is Cl, Br, F, I, or other good leaving group; and wherein E is attached to the remainder of ET by a biocleavable linker (a linker with a trigger) attached at either the phosphate, phosphonate, or hydroxy group.

In a preferred embodiment, X is O or CH₂ and Y is F, and the site of attachment is at the phosphate or phosphonate group.

In another preferred embodiment, E is comprised of a ligand which binds to TS and to which is attached a free radical generator that can covalently modify TS and thereby generates neoantigens. 1843U89 is an extremely potent inhibitor of TS with a Ki of 90 pM. The following references relate to this subject matter: Duch D. S., et al., “Biochemical and Cellular Pharmacology of 1843U89, a Novel Benzoquinazoline Inhibitor of Thymidylate Synthase,” Cancer Res, 53(4):810-8 (1993); Stout T. J.; Stroud R. M., “The Complex of the Anti-Cancer Therapeutic, BW1843U89, with Thymidylate Synthase at 2.0 a Resolution: Implications for a New Mode of Inhibition,” Structure, 4(1):67-77 (1996), the contents of which are incorporated herein by reference in their entirety.

In preferred embodiments (Eneo47 and Eneo48)), E comprises the following structures:

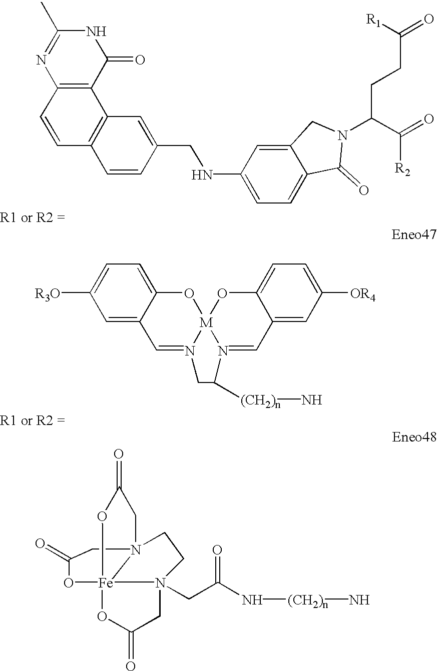

and wherein R₁ and R₂ can be OH or the structure as shown above, or the site of a trigger attachment; and wherein R3 and R4 are H , or bioreversible hydroxy masking groups, that can be converted in vivo into OH groups; and wherein n is 1-6, M is Cu (II) or Fe (II); and wherein E is linked to the remainder of ET by a trigger linked to one of the carboxylate or amino groups and wherein activation of said trigger liberates E from the remainder of ET. Suitable triggers have been described in the trigger section.

In a preferred embodiment (Eneo49) E comprises the following structure:

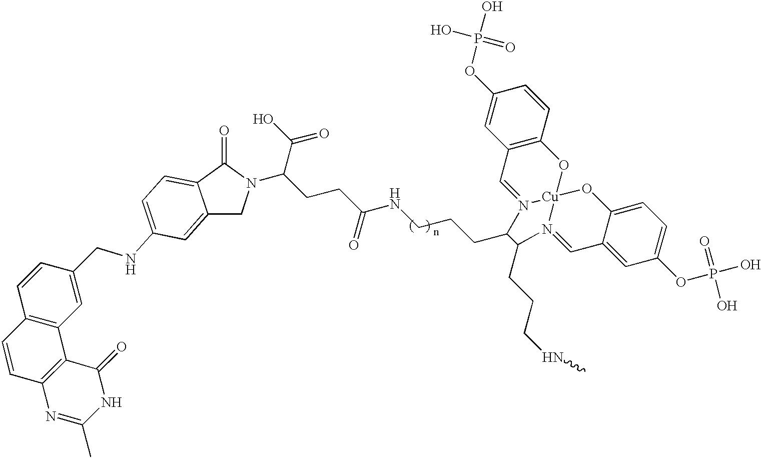

wherein n=0,1,2,3,4,5,6,7, 8,9,10 or about 10; the cooper is Cu(II); and the wavy line is the site of attachment to the remainder of ET preferably the E is attached to a trigger that is activated inside cells.

In a preferred embodiment (Eneo50), E is comprised of the following structure:

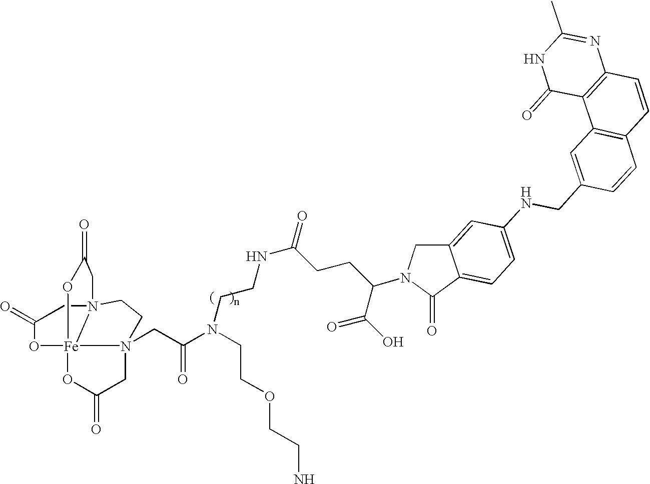

wherein n=0,1,2,3,4,5,6,7, 8,9,10 or about 10; the iron is Fe(II); and the wavy line is the site of attachment to the remainder of ET preferably the E is attached to a trigger that is activated inside cells.

In a preferred embodiment (Eneo 51) E is comprised of the following structure:

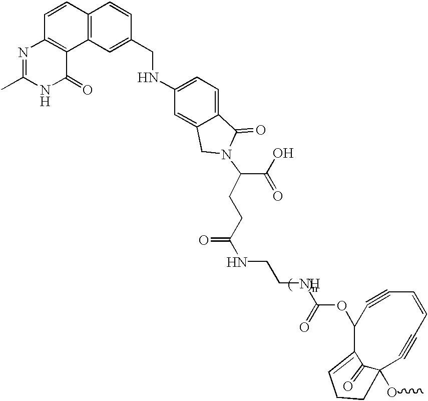

wherein n=0,1,2,3,4,5,6,7, 8,9,10 or about 10; and the wavy line is the site of attachment to the remainder of ET preferably the E is affached to a trigger that is activated inside cells.

Cathepsin B Targeted Neoantigens

Cathepsin B (Cat B) is a cysteine protease, which is over-expressed in a large number of tumors including: lung, colon, prostate, breast, gastric, glioblastoma, thyroid, melanoma, and ovarian cancers. Cat B plays an important role in tissue invasion and angiogenesis. Cat B over-expression is associated with poor patient outcome in a number of malignancies including: lung, brain, and breast cancers. A large number of inhibitors to Cat B have been developed. In addition, prodrugs designed to be activated by CAT B have been evaluated as anti-cancer drugs. However, to date there remains no satisfactory method to exploit Cat B as a tumor target. The following references relate to this subject matter: Foekens J. A., et al., “Prognostic Significance of Cathepsins B and L in Primary Human Breast Cancer,” J Clin Oncol, 16:1013-1021 (1998); Yan S., et al., “Cathepsin B and Human Tumor Progression,” Biol Chem, 379(2):113-23 (1998); Towatari T., et al., “Novel Epoxysuccinyl Peptides. A Selective Inhibitor of Cathepsin B, in Vivo,” FEBS, 280(2):311-315 (1991); Matsumoto K., et al., “X-Ray Crystal Structure of Papain Complexed with Cathepsin B-specific Covalent-type Inhibitor: Substrate Specificity and Inhibitory Activity,” Biochim Biophys Acta, 1383:93-100 (1998); Yamamoto A., et al., “Binding Mode of CA074, a Specific Irreversible Inhibitor, to Bovine Cathepsin B as Determined by X-Ray Crystal Analysis of the Complex,” J Biochem, 121:974-977 (1997); Palmer, J. T., et al., “Vinyl Sulfones as Mechanism-Based Cysteine Protease Inhibitors,” J Med Chem, 38:3193-3196 (1995), the contents of which are incorporated herein by reference in their entirety.

A large number of mechanism based suicide inhibitors of Cat B that colvalently modify the enzyme are known. The resulting neoantigen can be exploited in targeted immunotherapy.

In a preferred embodiment, E is a mechanism based suicide inhibitor of Cat B.

In a preferred embodiment (Eneo52), E comprises the following structure:

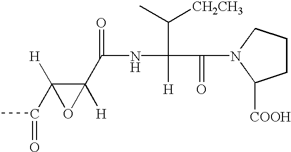

wherein the dotted line is the site of linker attachment to the remainder of the drug.

The resulting neoantigen is derived from the peptide of Cat B and the cysteine addition product to the epoxide ring.

Cathepsin L Targeted Neoantigens

Cathepsin L, like Cat B, is over-expressed by a number of malignancies. Mechansim based suicide inhibitors to Cat L are known and the resulting neoantigen can be exploited for targeted immunotherapy. The following references relate to this subject matter: Towatari T., et al., “Novel Epoxysuccinyl Peptides. A Selective Inhibitor of Cathepsin B, in Vivo,” FEBS, 280(2):311-315 (1991), the contents of which are incorporated herein by reference in their entirety.

In a preferred embodiment, E is a mechanism based suicide inhibitor or Cat L. In a preferred embodiment (Eneo53), E comprises the following structure:

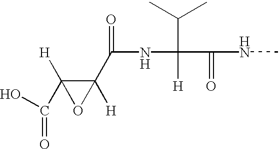

wherein the dotted line is the site of linker attachment to the remainder of the drug.

Cathepsin K Targeted Neoantigens

Cathepsin K is over-expressed in a number of malignancies and can play a role in the mechanisms of development of metastatic bone lesions. Cat K is inhibited by compounds of similar structure as for Cat L and can be employed in neoantigen targeted immunotherapy in a similar fashion.

Ribonucleotide Diphosphate Reductase Targeted Neoantigens

Ribonucleotide diphosphate reductase (RDPR) is a key enzyme in the synthesis of deoxyribonucleotides, which are essential precursors for DNA synthesis. RDPR is well recognized as a target of cancer therapy. Inhibition of the enzyme is central to the mechanism of action of a number of anti-cancer drugs including: hydroxyurea, Trimidox, (E)-2′-deoxy-2′-(fluoromethylene) cytidine, and gemcitabine. Current targeting of RDPR is associated with clinical toxicity and limited efficacy. Mechanism based suicide inhibitors that covalently modify RDPR are known. The resulting neoantigen can be employed in targeted immunotherapy. The following references relate to this subject matter: Baker C. H. et al., “2′-Deoxy-2′-methylenecytidine and 2′-deoxy-2′,2′-difluorocytidine 5′-diphosphates: Potent Mechanism-Based Inhibitors of Ribonucleotide Reductase,” J Med Chem, 34(6):1879-84 (1991); Salowe S. et al., “Alternative Model for Mechanism-Based Inhibition of Escherichia Coli Ribonucleotide Reductase by 2′-azido-2′-deoxyuridine 5′-diphosphate,” Biochemistry, 32(47):12749-60 (1993); Sjoberg B. M., et al., “A Substrate Radical Intermediate in the Reaction between Ribonucleotide Reductase from Escherichia Coli and 2′-Azido-2′-deoxynucleoside Diphosphates,” J Biol Chem, 258(13):8060-7 (1983); Szekeres T., et al., “Biochemical and Antitumor Activity of Trimidox, a New Inhibitor of Ribonucleotide Reductase,” Cancer Chemother Pharmacol, 34(1):63-6 (1994); Kang S. H.; Cho M. J., “Biological Activity and Phosphorylation of 2′-azido-2′-deoxyuridine and 2′-azido-2′-deoxycytidine,” Nucleosides Nucleotides, 17(6):1077-88 (1998); Cory J. G., “Ribonucleotide Reductase as a Chemotherapeutic Target,” Adv Enzyme Regul, 27:437-55 (1988); Bokemeyer C., et al., “Gemcitabine in Patients with Relapsed or Cisplatin-Refractory Testicular Cancer,” J Clin Oncol, 17(2):512 (1999); van der Donk W. A., et al., “Inactivation of Ribonucleotide Reductase by (E)-2′-fluoromethylene-2′-deoxycytidine 5′-diphosphate: a Paradigm for Nucleotide Mechanism-Based Inhibitors,” Biochemistry, 35(25):8381-91 (1996); Harris G. et al., “Mechanism of Inactivation of Escherichia Coli and Lactobacillus Leichmannii Ribonucleotide Reductases by 2′-chloro-2′-deoxynucleotides: Evidence for Generation of 2-methylene-3(2H)-furanone,” Biochemistry, 23(22):5214-25 (1984); Masuda N., et al., “Phase I and Pharmacologic Study of Oral (E)-2′-deoxy-2′-(fluoromethylene)cytidine: on a Daily×5-day Schedule,” Invest New Drugs, 16(3):245-54 (1998); Kang S. H. et al., “Synthesis and Biological Activity of bis(pivaloyloxymethyl) Ester of 2′-azido-2′-deoxyuridine 5′-monophosphate,” Nucleosides Nucleotides, 17(6):1089-98 (1998); Szekeres T. et al., “The Enzyme Ribonucleotide Reductase: Target For Antitumor and Anti-HIV Therapy,” Crit Reve Clin Lab Sci, 34(6):503-28 (1997); Takahashi T., et al., “Metabolism and Ribonucleotide Reductase Inhibition of (E)-2′-deoxy-2′-(fluoromethylene)cytidine, MDL 101, 731, in Human Cervical Carcinoma HeLa S3 Cells,” Cancer Chemother Pharmacol, 41(4):268-74 (1998); Salowe S. P., et al., “Products of the Inactivation of Ribonucleoside Diphosphate Reductase from Escherichia coli with 2′-Azido-2'deoxyuridine 5′-Diphosphate,” Biochemistry, 26:3408-3416 (1987); Thelander L., et al., “Active Site of Ribonucleoside Diphosphate Reductase from Escherichia Coli,” J Biological Chem, 251 (5):1398-1405 (1976); Bitonti A. J., et al., “Regression of Human Breast Tumor Xenografts in Response to (E)-2deoxy-2′(fluoromethylene)cytidine, and Inhibitor of Ribonucleoside Diphosphate Reductase,” Cancer Res, 54(6):1485-90 (1994); Bitonti A. J., et al., “Response of Human Colon and Prostate Tumor Xenografts to (E)-2′-deoxy-2′-(fluoromethylene) cytidine, an Inhibitor of Ribonucleotide Reductase,” Anti-cancer Res, 15(4):1179-82 (1995), the contents of which are incorporated herein by reference in their entirety.

In a preferred embodiment, E is a mechanism based suicide inhibitor of RDPR.

In a preferred embodiment (Eneo54), E comprises the following structure:

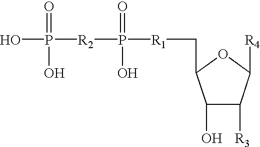

wherein R₁ and R₂ is O, CH₂, CHF, CF₂, and R₃ is azido, or a haolgen, and R₄ is a pyrimidine or purine base attached at N₁ or N₉ respectively; and wherein E is attached to the remainder of the targeted drug by a biocleavable linker (a linker with a trigger) attached at either the phosphate, phosphonate, or hydroxy group.

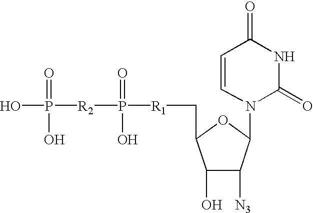

In a preferred embodiment (Eneo55), E comprises the following structure:

The interaction of RDPR with 2′ azido, and 2′ chloro nucleotide diphosphates covalently modifies the enzyme in an almost stochiometric mannner. There is evidence that the covalent modification is due to the generation of 2-methylene-3-(2H)-furanone in the active site. Regardless of the mechanism, the stable covalent modification of the enzyme can generate neoantigens that can be employed in targeted immunotherapy.

Trypsin Targeted Neoantigens

Trypsin is a serine protease, which is abnormally expressed by several important human malignancies including: ovarian cancer, gastric cancer, and lung cancer. A large number of mechanism based suicide inhibitors for trypsin are known and can be employed in neoantigen generation.

In a preferred embodiment, E is a mechanism based suicide inhibitor for trypsin.

In a preferred embodiment (Eneo56), E comprises the following structure:

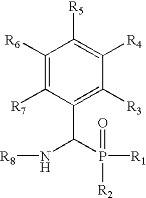

wherein either R₁ and R₂ is a good leaving group for nucleophilic substitution reactions at phosphorus; and wherein R₃, R₄, R₆, and R₇ can be H, Cl, Br, F, I, a lower alkyl group, a lower alkoxy group, OH, or NO₂; and wherein R₅ is an amidino group, a guanidino group, or a positively charged group, and R₈ is an oligopeptide or oligopeptide analog connected to the remainder of the drug.

In a preferred embodiment (Eneo57), E comprises the following structure:

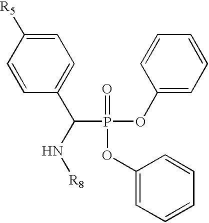

wherein R₅ is an amidino or guanidino group.

Protein Kinase A Targeted Neoantigens

Protein Kinase A type 1 (PKA) or cyclic AMP dependent protein kinase is a serine/threonine kinase which is over-expressed in a wide range of malignancies including: breast, colon, prostate, melanoma, renal cell, and lung cancer. PKA is an intracellular enzyme and also is released by tumors into the extracellular space. The massive overproduction of PKA by tumors leads to an average 10-fold increase in PKA type 1 serum levels in cancer patients. Estrogen receptor negative breast cancer cells show even greater over-expression of PKA than hormone dependent cells. Activation of epidermal growth factor, which is central to many malignancies, is accompanied by increased expression of PKA. A variety of inhibitors have been developed as potential anti-cancer drugs targeted to PKA. The expression of PKA is not limited to tumor cells and to date no technology exists to effectively utilize PKA as an anti-cancer target. PKA over-expression can be utilized as a targeting variable in targeted neoantigen immunotherapy. The following references relate to this subject matter: Kondrashin A., et al., “Cyclic Adenosine 3′:5′-Monophosphate-Dependent Protein Kinase on the External Surface of LS-174T Human Colon Carcinoma Cells,” Biochem, 38(1):172-9 (1999); Putz T., et al., “Epidermal Growth Factor (EGF) Receptor Blockade Inhibits the Action of EGF, Insulin-Like Growth Factor I, and a Protein Kinase A Activator on the Mitogen-Activated Protein Kinase Pathway in Prostate Cancer Cell Lines,” Cancer Res, 59(1):227-33 (1999); Nazareth L. V.; Weigel N. L., “Activation of the Human Androgen Receptor through a Protein Kinase A Signaling Pathway,” J Biol Chem, 271(33):19900-19907 (1996); Ciardiello F., et al., “Antitumor Activity of Combined Blockade of Epidermal Growth Factor Receptor and Protein Kinase A,” J Natl Cancer Inst, 88(23):1770-6 (1996); Ciardiello F.; Tortora G., “Interactions between the Epidermal Growth Factor Receptor and Type I Protein Kinase A: Biological Significance and Therapeutic Implications,” Clin Cancer Res, 4(4):821-8 (1998); Ciardiello F., et al., “Down-Regulation of Type I Protein Kinase A by Transfection of Human Breast Cancer Cells with an Epidermal Growth Factor Receptor Antisense Expression Vector,” Breast Cancer Res Treat, 47(1):57-62 (1998); Gordge P. C., et al., “Elevation of Protein Kinase A and Protein Kinase C Activities in Malignant as Compared with Normal Human Breast Tissue,” Eur J Cancer, 32A(12):2120-6 (1996); Cho Y. S., et al., “Extracellular Protein Kinase A as a Cancer Biomarker: its Expression by Tumor Cells and Reversal by a Myristate-Lacking Cα and RII^{β} Subunit Over-expression,” Proc Natl Acad Sci USA, 97(2):835-840 (2000), the contents of which are incorporated herein by reference in their entirety.

Balanol and its analogs are potent reversible inhibitors of PKA and Protein Kinase C (PKC). The analog 10″-deoxybalanol inhibits PKA with a Ki of 4 nm and PKC with a Ki of 640 nm. The following references relate to this subject matter: Gustafsson A. B., et al., “Differential and Selective Inhibition of Protein Kinase A and Protein Kinase C in Intact Cells by Balanol Congeners,” Molec Pharm, 56:377-382 (1999); Narayana N., et al., “Crystal Structure of the Potent Natural Product Inhibitor Balanol in Complex with the Catalytic Subunit of cAMP-Dependent Protein Kinase,” Biochemistiy, 38(8):2367-2376 (1999); Setyawan J., et al., “Inhibition of Protein Kinases by Balanol: Specificity within the Serine/Threonine Protein Kinase Subfamily,” Mol Pharmacol, 56(2):370-376 (1999), the contents of which are incorporated herein by reference in their entirety.

In a preferred embodiment, E is a ligand or a masked ligand for PKA to which is attached a triggerable free radical generator that irreversibly modifies PKA creating neoantigens.

In preferred embodiments (Eneo58 and Eneo59), E comprises the following structures:

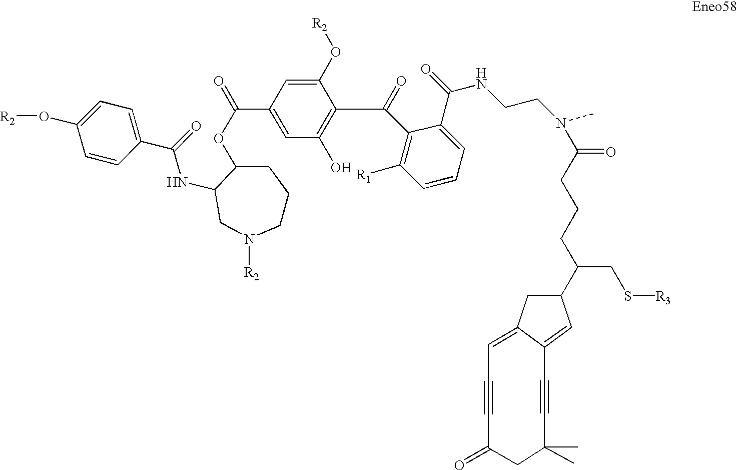

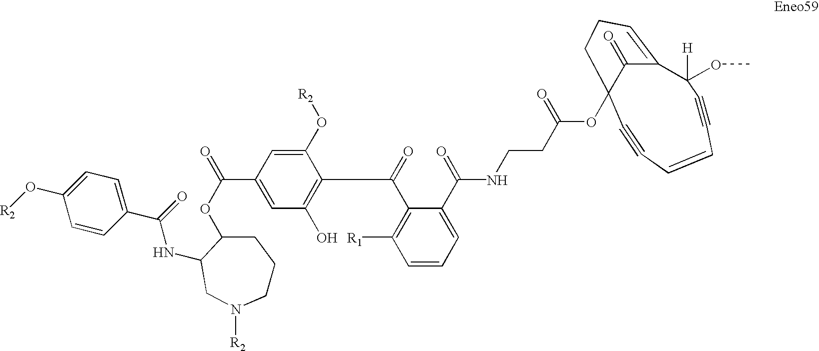

wherein R₁ is H, or OH, the dotted line is the site of linker attachment to the remainder of ET; and R₂ is H or trigger, which functions as a masking group; and wherein R₃ is trigger, or a bioreversible thiol protecting group such as an acyl group or a —S—R₄ where R₄ is any group such that the resulting disulfide is converted in cells to the free thiol.

Dihydrofolate Reductase Targeted Neoantigens

Dihydrofolate reductase (DHFR) catalyzes the reduction of 7,8-dihydrofolate to 5,6,7,8-tetrahydrofolate, which is essential for the synthesis of thymidylate, purines and glycine. DHFR is over-expressed in malignant cells and is under the control of the transcriptional factor E2F that plays a fundamental role in the biochemistry of malignancy. Inhibitors to DHFR such as methotrexate are routinely used as antineoplastic drugs. Gene amplification and over-expression of DHFR leads to resistance and impaired efficacy. In addition the metabolic block produced by inhibitors of DHFR can be by-passed by salvage pathways. The following references relate to this subject matter: Banerjee D., et al., “Molecular Mechanisms of Resistance to Antifolates, a Review,” Acta Biochem Pol, 42(4):457-64 (1995); Schuetz J. D., et al., “Transient Inhibition of DNA Synthesis By 5-Fluorodeoxyuridine Leads to Over-expression of Dihydrofolate Reductase with Increased Frequency of Methotrexate Resistance,” J Biol Chem, 263(16):7708-12 (1988); Blakley R. L; Benkovic S. J., Folates and Pterins, John Wiley & Sons, New York 1984; Piper J. R., “Methotrexate and Related Diaminoheterocycles,” in Caniam O. Foye , Ed., Cancer Chemotherapeutic Agents, American Chemical Society, Washington D.C., 1995. p. 97; Eastman H. B., et al., “Stimulation of Dihydrofolate Reductase Promoter Activity by Antimetabolic Drugs,” Proc Natl Acad Sci USA, 888:8572-8576 (1991); Muller H.; Helin K., “The E2F Transcription Factors: Key Regulators of Cell Proliferation,” Biochim Biophys Acta, 1470:M1-M12 (2000), the contents of which are incorporated herein by reference in their entirety.

In a preferred embodiment, E is comprised of a ligand, which binds to DHFR to which is attached a triggerable free radical generator. A large number of inhibitors of DHFR that are suitable ligands are known. The x-ray structures of some DHFR inhibitor complexes are known and provide detailed information about the solvent accessible sites on inhibitors to which a linker and free radical generator can be attached without compromising binding affinity to the enzyme. The DHFR inhibitor PT523 binds with a Ki of 0.35pM. The following references relate to this subject matter: Rosowsky A., et al., “Analogues of N^{α}-(4-Amino-4-deoxypteroyl)-N^{δ}-hemiphthaloyl-L-ornithine (PT523) Modified in the Side Chain: Synthesis and Biological Evaluation,” J Med Chem, 40:286-299 (1997); Johnson J. M., et al., “NMR Solution Structure of the Antitumor Compound PT523 and NADPH in the Ternary Complex with Human Dihydrofolate Reductase,” Biochemistry, 36:4399-4411 (1997); Rosowsky A., et al., “Synthesis and Biological Activity of N^{ω}-Hemiphthaloyl-α,ω-diaminoalkanoic Acid Analogues of Aminopterin and 3′,5-Dichloroaminopterin,” J Med Chem, 37:2167-2174 (1994), the contents of which are incorporated herein by reference in their entirety.

In preferred embodiments (Eneo60 and Eneo61), E comprises the following structures:

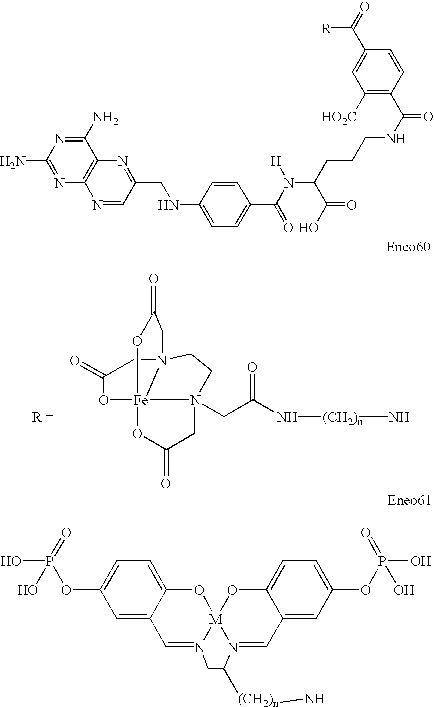

wherein n is 1-6, M is Cu (II) or Fe (II) and wherein E is linked to the remainder of ET by a trigger linked to one of the carboxylate or amino groups, which when activated, liberates E from the remainder of the drug. Suitable triggers have been described previously.

Src Kinase Targeted Neoantigens

Src Kinases (Src) are a family of nonreceptor tyrosine kinases, which are over-expressed in a number of malignancies including: breast, pancreatic, colon cancer and myeloid leukemia. Constituitive over-expression of Src is oncogenic. Src has been recognized as a potential anti-cancer target; and a large number of inhibitors to Src have been developed. The following references relate to this subject matter: Verbeek B. S., et al., “c-Src Protein Expression is Increased in Human Breast Cancer. An Immunohistochemical and Biochemical Analysis,” J Pathol, 180(4):383-8 (1996); Lutz M. P., et al., “Over-expression and Activation of the Tyrosine Kinase Src in Human Pancreatic Carcinoma,” Biochem Biophys Res Commun, 243(2):503-8 (1998); Cartwright C. A., et al., “pp60c-src Activation in Human Colon Carcinoma,” J Clin Invest, 83(6):2025-33 (1989); Brown M. T.; Cooper J. A., “Regulation, Substrates and Functions of Src,” Biochim Biophys Acta, 1287(2-3):121-49 (1996); Rosen N., et al., “Analysis of pp60c-Src Protein Kinase Activity in Human Tumor Cell Lines and Tissues,” J Biol Chem, 261(29):13754-9 (1986); Roginskaya V., et al., “Therapeutic Targeting of Src-Kinase Lyn in Myeloid Leukemic Cell Growth,” Leukemia, 13(6):855-61 (1999); Moasser M. M., et al., “Inhibition of Src Kinases by a Selective Tyrosine Kinase Inhibitor Causes Mitotic Arrest,” Cancer Res, 59(24):6145-52 (1999); Klutchko S. R., et al., “2-Substituted Aminopyrido[2,3-d]pyrimidin-7(8H)-ones. Structure-Activity Relationships Against Selected Tyrosine Kinases and in Vitro and in Vivo Anti-cancer Activity,” J Med Chem, 41:3276-3292 (1998); Hanke J. H., et al., “Discovery of a Novel, Potent, and Src Family-selective Tyrosine Kinase Inhibitor,” J Biol Chem, 271(2):695-701 (1996); Panek R. L., et al., “In Vitro Pharmacological Characterization of PD 166285, a New Nanomolar Potent in Broadly Active Protein Tyrosine Kinase Inhibitor,” J Pharm Exp Therap, 283(3):1433-1444 (1997), the contents of which are incorporated herein by reference in their entirety.

In a preferred embodiment, E is comprised of a ligand, which binds to Src and to which is attached a free radical generator. The generation of radicals can modify Src and create neoantigens. As mentioned above a large number of inhibitors, which bind tightly to Src, are known. Preferred embodiments are based on 2-Amino-8H-pyrido[2,3-d]pyrimidines analogs which bind at nanomloar levels to Src. The following references relate to this subject matter: Boschelli D. H., et al., “Synthesis and Tyrosine Kinase Inhibitory Activity of a Series of 2-Amino-8H-pyrido[2,3-d]pyrimidines: Identification of Potent, Selective Platelet-Derived Growth Factor Receptor Tyrosine Kinase Inhibitors,” J Med Chem, 41:4365-4377 (1998), the contents of which are incorporated herein by reference in their entirety.

In preferred embodiments (Eneo62 and Eneo63), E comprises the following structures:

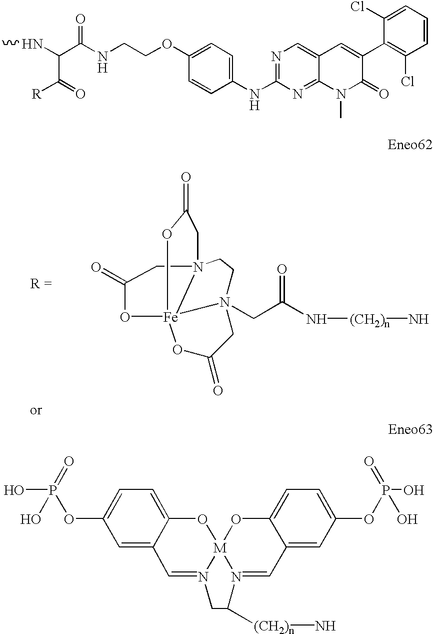

wherein n is 1-6, M is Cu (II) or Fe (II) and wherein E is linked to the remainder of the drug at the site indicated by the wavy line. In a preferred embodiment, E is linked to the remainder of the drug by a trigger, which is activated intracellularly and releases inside the cell the SRC binding free radical generator.

Platelet-derived Growth Factor Receptor Targeted Neoantigens

Platelet-derived growth factor receptors (PDGFR) are receptor tyrosine kinases which are over-expressed in numerous malignancies including ovarian, breast, prostate, pancreatic cancer, osteosarcoma, melanoma; and brain tumors. The constitutive expression of PDGFR tyrosine kinase activity is oncogenic. A large number of inhibitors to PDGFR tyrosine kinase have been developed as potential anti-cancer drugs. The following references relate to this subject matter: Coltrera M. D., et al., “Expression of Platelet-Derived Growth Factor B-Chain and the Platelet-Derived Growth Factor Receptor Beta Subunit in Human Breast Tissue and Breast Carcinoma,” Cancer Res, 55(12):2703-8 (1995); Henriksen R., et al., “Expression and Prognostic Significance of Platelet-Derived Growth Factor and its Receptors in Epithelial Ovarian Neoplasms,” Cancer Res, 53(19):4550-4 (1993); Barnhill R. L., et al., “Expression of Platelet-Derived Growth Factor (PDGF)-A, PDGF-B and the PDGF-Alpha Receptor, but not the PDGF-Beta Receptor, in Human Malignant Melanoma in Vivo,” Br J Dermatol, 135(6):898-904 (1996); Choft A., et al., “Tyrosine Kinases Expressed in Vivo by Human Prostate Cancer Bone Marrow Metastases and Loss of the Type 1 Insulin-Like Growth Factor Receptor,” Am J Pathol, 155(4):1271-9 (1999); Ebert M., et al., “Induction of Platelet-Derived Growth Factor A and B Chains and Over-Expression of their Receptors in Human Pancreatic Cancer,” Int J Cancer, 62(5):529-35 (1995); Shawver L. K, et al., “Inhibition of Platelet-Derived Growth Factor-Mediated Signal Transduction and Tumor Growth by N-[4-(trifluoromethyl)-phenyl]5-methylisoxazole-4-carboxamide,” Clin Cancer Res, 3(7):1167-77 (1997); Bhardwaj B., et al., “Localization of Platelet-Derived Growth Factor Beta Receptor Expression in the Periepithelial Stroma of Human Breast Carcinoma,” Clin Cancer Res, 2(4):773-82 (1996); Kawai T, et al., “Expression in Lung Carcinomas of Platelet-Derived Growth Factor and its Receptors,” Lab Invest, 77(5):431-6 (1997); Oda Y., et al., “Expression of Growth Factors and their Receptors in Human Osteosarcomas. Immunohistochemical Detection of Epidermal Growth Factor, Platelet-Derived Growth Factor and their Receptors: Its Correlation with Proliferating Activities and P53 Expression,” Gen Diagn Pathol, 141(2):97-103 (1995); Westermark B., et al., “Platelet-Derived Growth Factor in Human Glioma,” Glia, 15(3):257-63 (1995); Liu Y. C., et al., “Platelet-Derived Growth Factor is an Autocrine Stimulator for the Growth and Survival of Human Esophageal Carcinoma Cell Lines,” Exp Cell Res, 228(2):206-11 (1996); Showalter H. D.; Kraker A. J., et al., “Small Molecule Inhibitors of the Platelet-Derived Growth Factor Receptor, the Fibroblast Growth Factor Receptor, and Src Family Tyrosine Kinases,” Pharmacol Ther, 76(1-3):55-71 (1997); Palmer B. D., et al., “Structure-Activity Relationships for 5-Substituted 1-Phenylbenzimidazoles as Selective Inhibitors of the Platelet-Derived Growth Factor Receptor,” J Med Chem, 42(13):2373-2382 (1999); Boschelli D. H., et al., “Synthesis and Tyrosine Kinase Inhibitory Activity of a Series of 2-Amino-8H-pyrido[2,3-d]pyrimidines: Identification of Potent, Selective Platelet-Derived Growth Factor Receptor Tyrosine Kinase Inhibitors,” J Med Chem, 41(22):4365-4377 (1998), the contents of which are incorporated herein by reference in their entirety.

In a preferred embodiment, E is a ligand that binds to PDGFR to which is attached a free radical generator which induces neoantigen formation. The structures shown above regarding Src targeted neoantigen formation can also target PDGFR and Fibroblast growth factor receptor which is similarly over-expressed in numerous malignancies and within the scope of this patent.

Estrogen Receptor Targeted Neoantigens

Estrogen receptors (ER) are over-expressed in a number of malignancies including breast cancer, ovarian, endometrial and some prostate cancers. Tamoxifen an antiestrogen is routinely used in the treatment of receptor positive breast cancer. Unfortunately approximately 50% of pateints with estrogen receptor positive breast cancer do not respond to tamoxifen. Resistance to tamoxifen commonly develops despite the continued presence of the estrogen recptor. There have been a number of attempts to develop cytotoxins that are targeted towards the estrogen receptor. However there remains a need for a method to convert estrogen receptor expression into cytotoxicity. The following references relate to this subject matter: Macgregor J. I.; Jordan V. C., “Basic Guide to the Mechanisms of Antiestrogen Action,” Pharm Rev, 50(2):151-196 (1998); Bonkhoff H., et al., “Estrogen Receptor Expression in Prostate Cancer and Premalignant Prostatic Lesions,” Am J Pathol, 155:641-647 (1999); Devraj R., et al., “Design, Synthesis, and Biological Evaluation of Ellipticine-Estradiol Conjugates,” J Med Chem, 39(17):3367-3374 (1996); Roger P., et al., “Increased Immunostaining of Fibulin-1, an Estrogen-Regulated Protein in the Stroma of Human Ovarian Epithelial Tumors,” Am J Pathol, 153:1579-1588 (1998); Krohn K., et al., “Diethylstilbestrol-linked Cytotoxic Agents: Synthesis and Binding Affinity for Estrogen Receptors,” J Med Chem, 32(7):1532-8 (1989); Zablocki J. A., et al., “Estrogenic Affinity Labels: Synthesis, Irreversible Receptor Binding, and Bioactivity of Aziridine-Substituted Hexestrol Derivatives,” J Med Chem, 30(5):829-3; Leclercq G., “Guide-lines in the Design of New Antiestrogens and Cytotoxic-Linked Estrogens for the Treatment of Breast Cancer,” J Steroid Biochem, 19(1A):75-85 (1983); Kohle H., et al., “Hexestrol-Linked Cytotoxic Agents: Synthesis and Binding Affinity for Estrogen Receptors,” J Med Chem, 32(7):1538-47 (1989); Brinkman A., et al., “BCAR1, a Human Homologue of the Adapter Protein p130Cas, and Antiestrogen Resistance in Breast Cancer Cells,” J Nat Cancer Inst, 92(2):112-120 (2000); V. Craig Jordan, “How is Tamoxifen's Action Subverted,” J Nat Cancer Inst, 92(2):92-94 (2000); Rink S. M., et al., “Synthesis and Biological Activity of DNA Damaging Agents that Form Decoy Binding Sites for the Estrogen Receptor,” Proc Natl Acad Sci, 93:15063-15068 (1996); Kuduk S. D., et al., “Synthesis and Evaluation of Geldanamycin-Estradiol Hybrids,” Bioorg Med Chem Lett, 9(9):1233-8 (1996); Robertson D. W., et al., “Tamoxifen Aziridines: Effective Inactivators of the Estrogen Receptor,” Endocrinology, 109(4):1298-300 (1981), the contents of which are incorporated herein by reference in their entirety.

In a preferred embodiment, E is comprised of a ligand that binds to the estrogen receptors and to which is attached a moiety capable of irreversibly modifying the ER and generating neoantigens. A large number of compounds such as 4-hydroxy tamoxifen and raloxifene, bind estrogen receptors with high affinity and by known structural mechanisms. The following references relate to this subject matter: Macgregor J. I.; Jordan V. C., “Basic Guide to the Mechanisms of Antiestrogen Action,” Pharm Rev, 50(2):151-196 (1998); Shiau A. K. et al., “The Structural Basis of Estrogen Receptor/Coactivator Recognition and the Antagonism of this Interaction by Tamoxifen,” Cell, 95(7):927-37 (1998), the contents of which are incorporated herein by reference in their entirety.

In preferred embodiments (Eneo64 and Eneo65), E comprises the following structures:

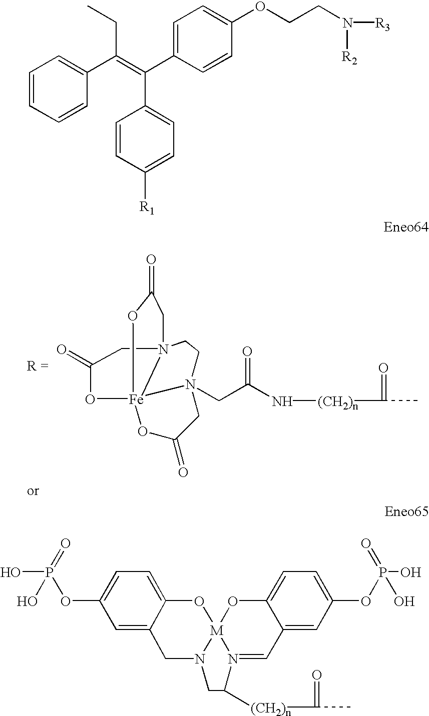

wherein R₁ is H, or OH, or the site of attachment of a trigger connected to the remainder of the targeted drug such that activation of the trigger liberates the tamoxifen analog, and wherein R₂ is H, methyl, or the site of attachment to the remainder of the targeted drug; and wherein n=1 to 6; and M is Cu(II) of Fe(II).

Another preferred set of structures is based on raloxifene. The following references relate to this subject matter: Palkowitz A. D., et al., “Discovery and Synthesis of [6-Hydroxy-3-[4-[2-(1-piperidinyl)ethoxy]phenoxy]-2-(4-hydroxyphenyl)]benzo[b]thiophene: A Novel, Highly Potent, Selective Estrogen Receptor Modulator,” J Med Chem, 40(10):1407-1416 (1997), the contents of which are incorporated herein by reference in their entirety.

These preferred embodiments (Eneo66 and Eneo67) of E are shown below:

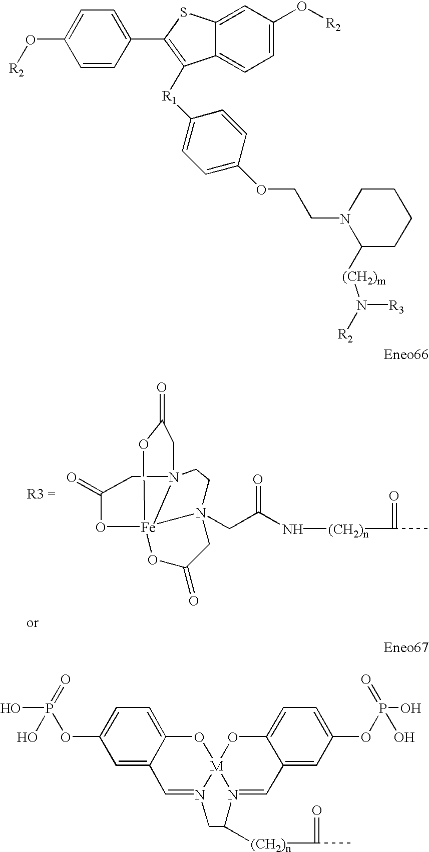

wherein R₁ is CO, CH₂, S, O, or NH, and m=1 to 6; n=1,2,3,4,5,6 or about 6, M is Cu(II) or Fe(II); R2 is H, or the site of attachment of a trigger connected to the remainder of the targeted drug such that activation of the trigger liberates the raloxifene analog.

Other preferred embodiments are based on the ability of tamoxifen aziridine and related compounds to efficiently affinity label ER by alkylation of a cysteine residue. The following references relate to this subject matter: Katzenellenbogen J. A., et al., “Efficient and Highly Selective Covalent Labeling of the Estrogen Receptor with [³H]Tamoxifen Aziridine,” J Biol Chem, 258(6):3487-3495 (1983); Harlow K. W., et al., “Identification of Cysteine 530 as the Covalent Attachment Site of an Affinity-labeling Estrogen (Ketononestrol Aziridine) and Antiestrogen (Tamoxifen Aziridine) in the Human Estrogen Receptor,” J Biol Chem, 264(29):17476-17485 (1989); Reese J. C.; Katzenellenbogen B. S., “Mutagenesis of Cysteines in the Hormone Binding Domain of the Human Estrogen Receptor,” 266(17):10880-10887 (1991); Aliau S., et al., “Cysteine 530 of the Human Estrogen Receptor α is the Main Covalent Attachment Site of 11β-(Aziridinylalkoxyphenyl)estradiols,” Biochemistry, 38:14752-14762 (1999), the contents of which are incorporated herein by reference in their entirety.

In these embodiments, E is comprised of an ER binding ligand to which is coupled a latent alkylating agent which is unmasked upon activation of a trigger.

In a preferred embodiment (Eneo68 and Eneo69), E comprises the following structure:

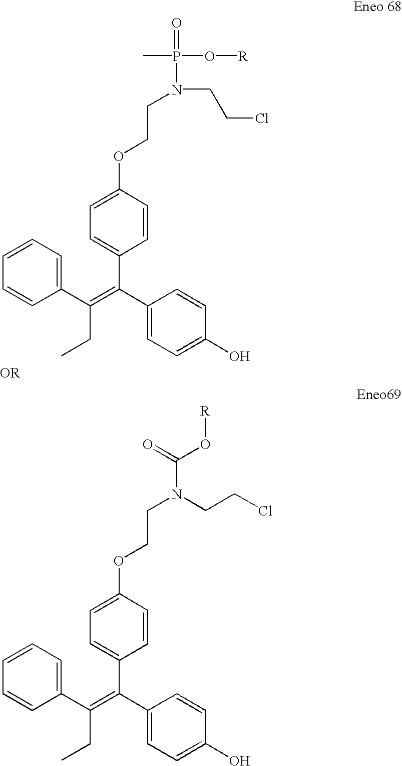

wherein R is a trigger attached to the remainder of the targeted drug such that activation of the trigger cleaves the phophoester or carbamate generating an electrophilic species. A wide variety of suitable triggers have been described elsewhere in this patent.

P-Glycoprotein Targeted Neoantigens

P-glycoprotein (p-G) is a protein that pumps a diverse range of drugs out of cells and is a major mediator of resistance to anti-cancer drugs. p-G is constitutively over-expressed in a large number of malignancies. In addition, p-G over-expression in human tumors may be rapidly induced by exposure to anti-cancer drugs. A large number of componds that inhibit p-G have been developed and some are in clinical trials as chemosensitzers. The following references relate to this matter: Ambudkar S. V., et al., “Biochemical, Cellular, and Pharmacological Aspects of the Multi-drug Transporter,” Annu Rev Pharmacol Toxicol 39:361-398 (1999); and Sutoh I., et al., “Concurrent Expressions of Metallothionein, Glutathione S-transferase-pi, and P-glycoprotein in Colorectal Cancers,” Dis Colon Rectum, 43(2):221-32 (2000); and Chan H. S., et al., “Immunohistochemical Detection of P-glycoprotein: Prognostic Correlation in Soft Tissue Sarcoma of Childhood,” J Clin Oncol, 8:689-704 (1990); and Yang J. M., et al., “Inhibitory Effect of Alkylating Modulators on the Function of P-glycoprotein,” Oncol Res, 9(9):477-84 (1997); and Callaghan R; Higgins C. F., “Interaction of Tamoxifen with the Multi-drug Resistance P-glycoprotein,” Br J Cancer, 71(2):294-9 (1995); and Hofmann J., et al., “Mechanism of Action of Dexniguldipine-Hcl (B8509-035), A New Potent Modulator of Multi-drug Resistance,” Biochem Pharmacol, 49(5):603-9 (1995); and Loo T. W; Clarke D. M., “Merck Frost Award Lecture 1998. Molecular Dissection of the Human Multi-drug Resistance P-glycoprotein,” Biochem Cell Biol, 77(1):11-23 (1999); and Fracasso P. M., et al., “Phase I Study of Paclitaxel in Combination with a Multi-drug Resistance Modulator, PSC 833 (Valspodar), in Refractory Malignancies,” J Clin Oncol, 18(5):1124 (2000); and Tidefelt U., et al., “P-Glycoprotein Inhibitor Valspodar (PSC 833) Increases the Intracellular Concentrations of Daunorubicin In Vivo in Patients with P-Glycoprotein-Positive Acute Myeloid Leukemia,” J Clin Oncol, 18(9):1837-1844 (2000); and Abolhoda A., et al., “Rapid Activation of MDRI Gene Expression in Human Metastatic Sarcoma after In Vivo Exposure to Doxorubicin,” Clin Cancer Res, 5(11):3352-6 (1999); and Traunecker H. C., et al., “The Acridonecarboxamide GF120918 Potently Reverses P-Glycoprotein-Mediated Resistance in Human Sarcoma MES-Dx5 Cells,” Br J Cancer, 81(6):942-51 (1999); and Martin C., et al., “The Molecular Interaction of the High Affinity Reversal Agent XR9576 with P-glycoprotein,” Br J Pharmacol, 128(2):403-11 (1999); and the contents are hereby incorporated by reference in their entirety.

In a preferred embodiment E is a ligand, which irreversibly modifies p-G and induces neoantigen formation. A large number of compounds are known which bind with high affinity to p-G. In a preferred embodiment, E is comprised of a p-G binding compound attached to an alkylating agent or a free radical generator. A preferred embodiment is based on the ability of tamoxifen aziridine to covalently bind to p-G. The following reference relates to this matter: Safa A. R., et al., “Tamoxifen Aziridine, a Novel Affinity Probe for P-glycoprotein in Multi-drug Resistant Cells,” Biochem Biophys Res Commun, 202(1):606-12 (1994), and the contents are hereby incorporated by reference in their entirety.

In a preferred embodiment(Eneo70), E has the following structure:

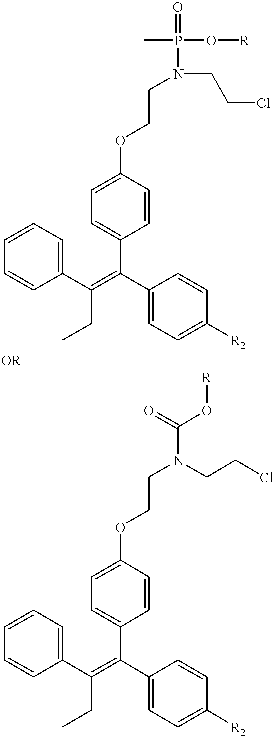

wherein R is a trigger attached to the remainder of the targeted drug such that activation of the trigger cleaves the phophoester or carbamate generating an electrophilic species. A wide variety of suitable triggers have been described elsewhere in this patent. R₂ is H, OH, or O—CH₃.

Prostatic Acid Phosphatase Targeted Neoantigens

Prostatic acid phosphatase (PAP) is a marker for prostatic epithelial cells, which is expressed in prostate cancer. PAP has been recognized as a potential target for immunotherapy of prostate cancer. PAP is relatively nonspecific and is able to hydrolyse a broad range of phosphate esters including even large proteins with phosphorylated residues. The following references relate to this matter: Peshwa M. V., et al., “Induction of Prostate Tumor-Specific CD8+ Cytotoxic T-Lymphocytes in Vitro using Antigen-Presenting Cells Pulsed with Prostatic Acid Phosphatase Peptide,” Prostate, 36(2):129-38 (1998); Ljung G., et al., “Characterization of Residual Tumor Cells Following Radical Radiation Therapy for Prostatic Adenocarcinoma; Immunohistochemical Expression of Prostate-Specific Antigen, Prostatic Acid Phosphatase, and Cytokeratin 8,” Prostate, 31(2):91-7 (1997); Mori K.; Wakasugi C., “Immunocytochemical Demonstration of Prostatic Acid Phosphatase: Different Secretion Kinetics between Normal, Hyperplastic and Neoplastic Prostates,” J Urol, 133(5):877-83 (1985); Fong L., et al., “Induction of Tissue-Specific Autoimmune Prostatitis with Prostatic Acid Phosphatase Immunization: Implications for Immunotherapy of Prostate Cancer,” J Immunol, 159(7):3113-7 (1997); Workman P., “Inhibition of Human Prostatic Tumour Acid Phosphatase by N,N-p-di-2-chloroethylaminophenol, N,N-p-di-2-chloroethylaminophenyl Phosphate and Other Difunctional Nitrogen Mustards,” Chem Biol Interact, 20(1):103-12 (1978); Sinha A. A., et al., “Immunocytochemical Localization of an Immunoconjugate (Antibody IgG against Prostatic Acid Phosphatase Conjugated to 5-fluoro-2′-deoxyuridine) in Human Prostate Tumors,” Anti-cancer Res, 18(3A): 1385-92 (1998); Warhol M. J.; Longtine J. A., “The Ultrastructural Localization of Prostatic Specific Antigen and Prostatic Acid Phosphatase in Hyperplastic and Neoplastic Human Prostates,” J Urol, 134(3):607-13 (1985); Lee H., et al., “Endogenous Protein Substrates for Prostatic Acid Phosphatase in Human Prostate,” Prostate, 19(3):251-63 (1991); Lin M. F.; Clinton G. M., “Human Prostatic Acid Phosphatase has Phosphotyrosyl Protein Phosphatase Activity,” Biochem J, 235(2):351-7 (1986); Wasylewska E., et al., “Phosphoprotein Phosphatase Activity of Human Prostate Acid Phosphatase,” Acta Biochim Pol, 30(2):175-84 (1983); and the contents are hereby incorporated by reference in their entirety.

In a preferred embodiment E is a group which irreversibly modifies PAP and generates neoantigens. Benzylaminophosphonic acid derivatives inhibit PAP reversibly at nanomolar concentrations. The following reference relates to this matter: Beers S. A., et al., “Phosphatase Inhibitors-III. Benzylaminophosphonic Acids as Potent Inhibitors of Human Prostatic Acid Phosphatase,” Bioorg Med Chem, 4(10):1693-701 (1996), and the contents is hereby incorporated by reference in its entirety.

In a preferred embodiment E is comprised of a free radical generator coupled to an inhibitor of PAP.

In a preferred embodiment (Eneo73) E is comprised of the structure shown below:

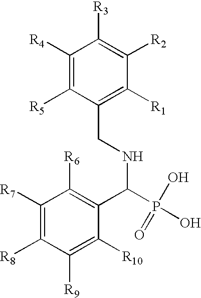

wherein a linker(s) coupled to a free radical generator and the remainder of the targeted drug is attached directly or indirectly to a site selected from R₁- to R₁₀, and wherein R₁-R₁₀ may be inert groups which do not interfere with the binding to PAP. In preferred embodiments R₁-R₁₀ are H, OH, a Cl, Br, F, I, nitro, a phenol, a lower alkoxy group, an amino group, a lower alkyl group, —CO₂H, and —CO₂R₁₁; wherein R₁₁ is a lower alkyl group; —CONHR₁₂; wherein NHR₁₂ is an amino acid or oligopeptide.

In preferred embodiments (Eneo74 and Eneo75), E is comprised of the structures shown below:

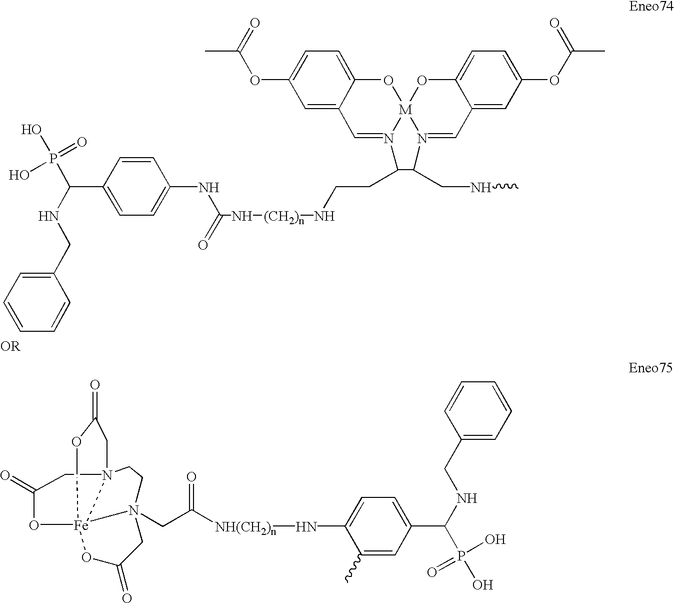

wherein n=1,2,3,4,5,6 or about 6, M is Cu(II) or Fe(II), and the wavy line is the site of attachment to the remainder of the targeted drug.

Matrix Metalloprotease Targeted Neoantigens

Matrix metalloproteases are enzymes which degrade connective tissue and which are over-expressed by a large number of tumors and stroma of tumors. There have been an enormous number of inhibitors to matrix metalloproteases developed as potential anti-cancer drugs. However, inhibition of MMP activity does not typically produce cytotoxicity. At the present time there are no known methods to convert the over-expression of MMPs into selective tumor toxicity. The following reference relate to this matter: Nelson A. R., et al., “Matrix Metalloproteinases: Biologic Activity and Clinical Implications,” J Clin Oncol, 18(5):1135 (2000); and Whittaker M., et al., “Design and Therapeutic Application of Matrix Metalloproteinase Inhibitors,” Chem Rev, 99:2735-2776 (1999); and Curran S.; Murray G. I., “Matrix Metalloproteinases in Tumour Invasion and Metastasis,” J Pathol, 189(3):300-308 (1999); and the contents are hereby incorporated by reference in their entirety.

In a preferred embodiment, E is a ligand, which binds to a matrix metalloprotease and irreversibly modifies the enzyme generating neoantigens.

Matrix Metalloproteinase 7 Targeted Neoantigens

Matrix Metalloproteinase 7 (MMP-7 or Matrilysin) is a protease, which is constitutively produced by exocrine epithelial cells. MMP-7 is over-expressed by the tumor cells of a wide range of malignancies including ovarian, gastric, prostate, colorectal, endometrial, gliomas, and breast cancer. MMP-7 contrasts with many other metalloproteases which are over-expressed by tumor stromal elements rather then the tumor cells. At the present time there are no known methods to convert the over-expression of MMP-7 into selective tumor toxicity. The following references relate to this matter: Yamamoto H., et al., “Association of Matrilysin Expression with Recurrence and Poor Prognosis in Human Esophageal Squamous Cell Carcinoma,” Cancer Res, 59(14):3313-6 (1999); Adachi Y., et al., “Contribution of Matrilysin (MMP-7) to the Metastatic Pathway of Human Colorectal Cancers,” Gut, 45(2):252-8 (1999); Yamashita K, et al., “Expression and Tissue Localization of Matrix Metalloproteinase 7 (Matrilysin) in Human Gastric Carcinomas. Implications for Vessel Invasion and Metastasis,” Int J Cancer, 79(2):187-94 (1998); Pacheco M. M., et al., “Expression of Gelatinases A and B, Stromelysin-3 and Matrilysin Genes in Breast Carcinomas: Clinico-Pathological Correlations,” Clin Exp Metastasis, 16(7):577-85 (1998); Hashimoto K., et al., “Expression of Matrix Metalloproteinase-7 and Tissue Inhibitor of Metalloproteinase-1 in Human Prostate,” J Urol, 160(5):1872-6 (1998); Mori M., et al., “Over-expression of Matrix Metalloproteinase-7 mRNA in Human Colon Carcinomas,” Cancer, 75(6 Suppl):1516-9 (1995); Honda M., et al., “Matrix Metalloproteinase-7 Expression in Gastric Carcinoma,” Gut, 39(3):444-8 (1996); Nakano A., et al., “[Increased Expression of Gelatinases A and B, Matrilysin and TIMP-1 Genes in Human Malignant Gliomas],” Nippon Rinsho, 53(7):1816-21 (1995); Knox J. D., et al., “Matrilysin Expression in Human Prostate Carcinoma,” Mol Carcinog, 15(1):57-63 (1996); Adachi Y., et al., “Matrix Metalloproteinase Matrilysin (MMP-7) Participates in the Progression of Human Gastric and Esophageal Cancers,” Int J Oncol, 13(5):1031-5 (1998); Ueno H., et al., “Enhanced Production and Activation of Matrix Metalloproteinase-7 (Matrilysin) in Human Endometrial Carcinomas,” Int J Cancer, 84(5):470-7 (1999); Barille S., et al., “Production of Metalloproteinase-7 (Matrilysin) by Human Myeloma Cells and its Potential Involvement in Metalloproteinase-2 Activation,” J Immunol, 163(10):5723-8 (1999); Senota A., et al., “Relation of Matrilysin Messenger RNA Expression with Invasive Activity in Human Gastric Cancer,” Clin Exp Metastasis, 16(4):313-21 (1998); Saarialho-Kere U. K., et al., “Matrix Metalloproteinase Matrilysin is Constitutively Expressed in Adult Human Exocrine Epithelium,” J Invest Dermatol, 105(2):190-6 (1995);. Tanimoto H., et al., “The Matrix Metalloprotease Pump-1 (MMP-7, Matrilysin): A Candidate Marker/Target for Ovarian Cancer Detection and Treatment,” Tumour Biol, 20(2):88-98 (1999); and the contents are hereby incorporated by reference in their entirety.

In a preferred embodiment, E is a ligand for MMP-7 to which is attached a triggerable free radical generator. A large number of potent reversible ligands are known that reversibly inhibit MMP-7. These ligands will tether the free radical generator to MMP-7 and focus free radical induced protein modification leading to the generation of MMP-7 based neoantigens. The following references relate to this matter: Pratt L. M., et al., “The Synthesis of Novel Matrix Metalloproteinase Inhibitors Employing the Ireland-Claisen Rearrangement,” Bioorg Med Chem Lett, 8:1359-1364 (1998); and Abramson S. R., et al., “Characterization of Rat Uterine Matrilysin and Its cDNA,” J Biological Chem, 270(27):16016-16022 (1995); and Nelson A. R., et al., “Matrix Metalloproteinases: Biologic Activity and Clinical Implications,” J Clin Oncology, 18(5):1135-1149 (2000); and Whittaker M., et al., “Design and Therapeutic Application of Matrix Metalloproteinase Inhibitors,” Chem Rev, 99:2735-2776 (1999) and the contents are hereby incorporated by reference in their entirety.

In a preferred embodiment, E is comprised of a MMP-7 ligand of the following structure:

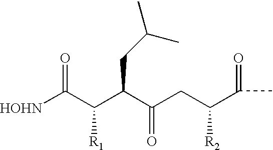

wherein the dotted line is the site of attachment or linker attachment to the triggerable free radical generator and wherein R₁ is hydroxy, methyl, ethyl, isopropyl, cyclopentyl, 3-(tetrahydrothiophenyl), or thiopen-2-ylthiomethyl; and wherein R₂ is benzyl, t-butyl, or isopropyl.

In preferred embodiments (Eneo76-Eneo79), E has the following structures:

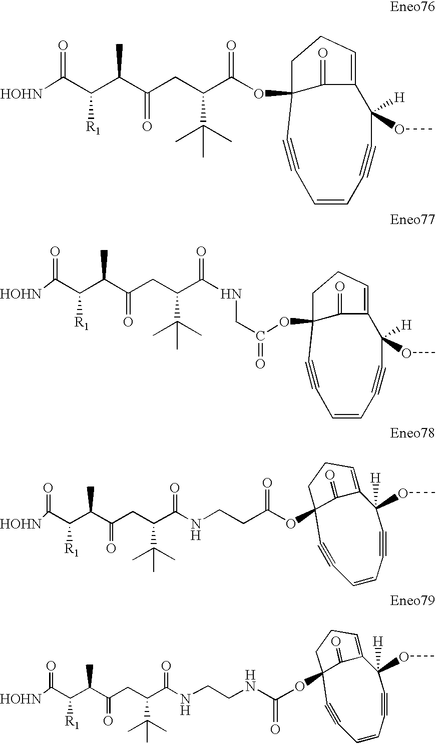

and wherein, the dotted line indicates the site of attachment of the remainder of the drug.

As discussed previously, addition of a thiol to the double bond of the free radical generator will trigger the formation of a reactive diradical that will react with the MMP-7 and generate neoantigens.

In another preferred embodiment (Eneo80), E has the following structure:

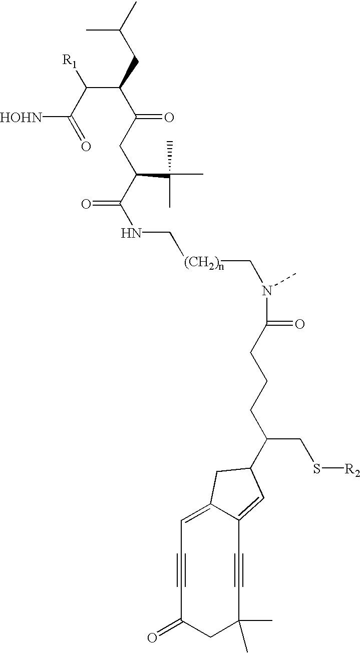

wherein the dofted line is the site of linker attachment to the remainder of ET and wherein R₁ is hydroxy, methyl, ethyl, isopropyl, cyclopentyl, 3-(tetrahydrothiophenyl), or thiopen-2-ylthiomethyl-; and wherein R₂ is an acyl group, or R₂ is a clock-like time delay trigger, or a bioreversible thiol protecting group such as —S—R₄, where R₄ is a group such that the disulfide is reduced to the thiol by cells; and wherein N=0,1,2,3,4,5,6 or about 6.

In other preferred embodiments (Eneo81 and Eneo82), E has the following structures:

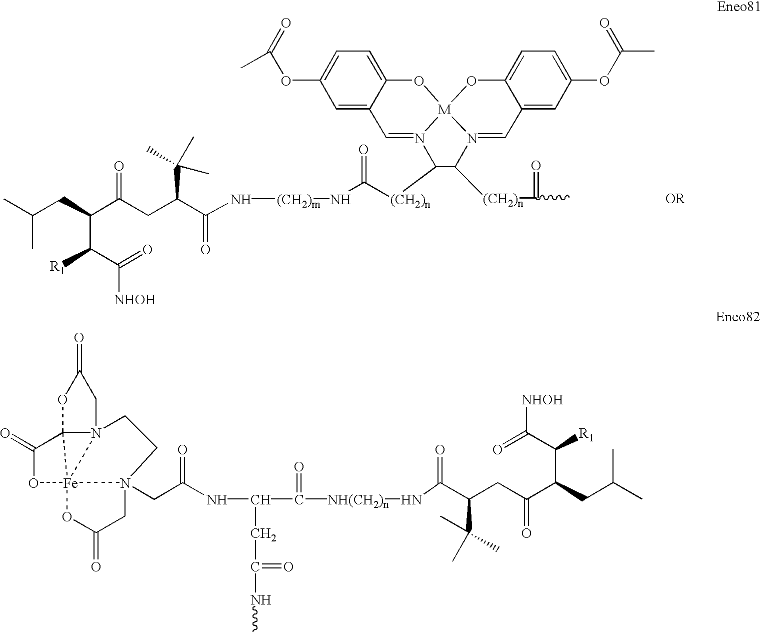

wherein n=1,2,3,4,5,6 or about 6; m=2,3,4,5,6 or about 6; the wavy line is the site of linker attachment to the remainder of ET; and wherein R₁ is hydroxy, methyl, ethyl, isopropyl, cyclopentyl, 3-(tetrahydrothiophenyl), or thiopen-2-ylthiomethyl; and wherein M is Cu(II) or Fe(II).

Neoantigen Formation Targeted to MMP1, 2, 3, 9 and Membrane Type 1 MMP.

MMP 1, 2, 3, 9 and membrane type MMP 1(MT-MMP-1) are all over-expressed in a wide variety of malignancies.

Similarities in the active site of these enzymes allow for targeting with a common family of ligands. The neoantigens generated and required for sensitization however should be unique for each enzyme. Compounds of the following structure bind reversibly to MMP 1, 2, 3, 9 and membrane type MMP with IC₅₀

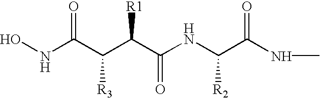

in the nanomolar to subnanomolar range.

wherein R₁ is —CH₂CH(CH₃)₂, —(CH₂)₂C₆H₅, —(CH₂)₃C₆H₅ , n-butyl, n-hexyl, or n-octyl; R2 is C₆H₅, - - - C₆H₁₁, —C(CH₃)₃, (indol-3-yl)methyl, —CH₂C₆H₅, (5, 6, 7, 8,-terahydro-1-napthyl)methyl, —CH(CH₃)₂, 1-(napthyl)methyl, 3-(napthyl)methyl, (quinolyl)methyl, 3-(quinolyl)methyl, 3-pyridylmethyl, 4-pyridylmethyl, or t-butyl; and R3 is H, OH, methyl, 2-thienylthiomethyl, or allyl.

The following references relate to this matter:Yamamoto M., et al., “Inhibition of Membrane-Type 1 Matrix Metalloproteinase by Hydroxamate Inhibitors: An Examination of the Subsite Pocket,” J Med Chem, 41:1209-1217 (1998).; and Curtin M. L., et al., “Broad Spectrum Matrix Metalloproteinase Inhibitors: An Examination of Succinamide Hydroxamate Inhibitors with P₁C_{α} Gem-Disubstitution,” Biorg Med Chem Lett, 8:1443-1448 (1998); and Levy D. E., et al., “Matrix Metalloproteinase Inhibitors: A Structure-Activity Study,” J Med Chem, 41:199-223 (1998) and their contents are hereby incorporated by reference in their entirety.

In a preferred embodiment, E is comprised of a ligand which binds to MMP1, 2, 3, 9 or MT-MMP-1 to which is attached a free radical generator. In preferred embodiments (Eneo8-Eneo86), E has the following structures:

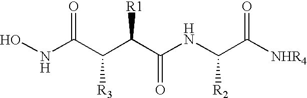

wherein R₄ has the following structure:

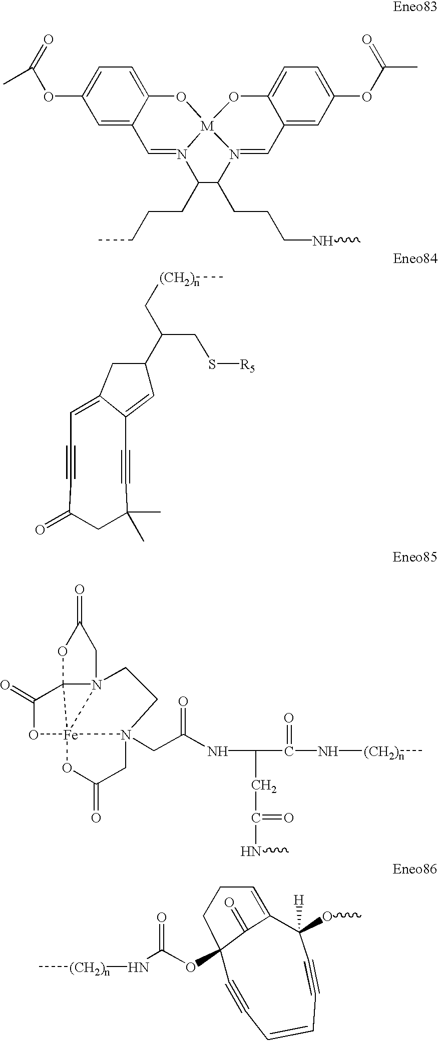

and wherein the dotted line is the site of attachment to the N of the MMP ligand the wavy line is the site of attachment to the remainder of the targeted drug ; and wherein R₁ is —CH₂CH(CH₃)₂, —(CH₂)₂C₆H₅, —(CH₂)₃C₆H₅, n-butyl, n-hexyl, or n-octyl. R₂ is C₆H₅, - - - C₆H₁₁, —C(CH₃)₃, (indol-3-yl)methyl, —CH₂C₆H₅, (5,6,7,8,-terahydro-1-napthyl)methyl, —CH(CH₃)₂, 1-(napthyl)methyl, 3-(napthyl)methyl, 1-(quinolyl)methyl, 3-(quinolyl)methyl, 3-pyridylmethyl, 4-pyridylmethyl, or t-butyl; and R₃ is H, OH, methyl, 2-thienylthiomethyl, or allyl; and wherein n=1,2,3,4,5,6 or about 6 and M is Cu(II) of Fe(II); and wherein R₅ is an acyl group, or R₅ is a clock-like time delay trigger, or a bioreversible thiol protecting group such as —S—R₆; where R₆ is any group such that the disulfide is reduced to the thiol by cells.

In preferred embodiments (Eneo87), E has the following structures:

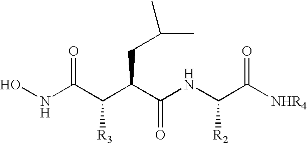

wherein R₂ is benzyl and R₃ is 2-thienylthiomethyl; or wherein R₂ is 5,6,7,8,-terahydro-1-napthyl)methyl, and R₃ is methyl; or wherein R₂ is t-butyl and R₃ is OH; or wherein R₂ is H and R₃ is (indol-3-yl)methyl; and wherein R₄ is as shown above.

Another preferred embodiment is based on diphenlyether sulfone inhibitors of MMP's which are highly active against MMP2, 3, 9, 12, and 13. The following references relate to this matter: U.S. Pat. No. 5,932,595, Aug. 3, 1999 Bender et al., “Matrix Metalloprotease Inhibitors”; and Lovejoy B., et al., “Crystal Structures of MMP-1 and -13 Reveal the Structural Basis for Selectivity of Collagenase Inhibitors,” Nat Struct Biol, 6(3):217-21 (1999)and; Botos I., et al., “Structure of Recombinant Mouse Collagenase-3 (MMP-13),” J Mol Biol, 292:837-844 (1999), and their contents are hereby incorporated by reference in their entirety.

MMP 13 is an attractive target for neoantigen formation as it is over-expressed in a wide range of malignancies.

Pendas A. M., et al., “An Overview of Collagenase-3 Expression in Malignant Tumors and Analysis of its Potential Value as a Target in Antitumor Therapies,” Clin Chim Acta, 291(2):137-55 (2000); and Shalinsky D. R., et al., “Broad Antitumor and Antiangiogenic Activities of AG3340, a Potent and Selective MMP Inhibitor Undergoing Advanced Oncology Clinical Trials,” Ann NY Acad Sci, 878:236-70 (1999); and Johansson N., et al., “Collagenase-3 (MMP-13) is Expressed by Tumor Cells in Invasive Vulvar Squamous Cell Carcinomas,” Am J Pathol, 154(2):469-80 (1999); and Barmina O. Y., et al., “Collagenase-3 Binds to a Specific Receptor and Requires the Low Density Lipoprotein Receptor-Related Protein for Internalization,” J Biol Chem, 274(42):30087-93 (1999); and Cazorla M., et al., “Collagenase-3 Expression is Associated with Advanced Local Invasion in Human Squamous Cell Carcinomas of the Larynx,” J Pathol, 186(2):144-150 (1998); and Balbin M., et al., “Expression and Regulation of Collagenase-3 (MMP-13) in Human Malignant Tumors,” APMIS, 107(1):45-53 (1999); and Johansson N., et al., “Expression of Collagenase-3 (Matrix Metalloproteinase-13) in Squamous Cell Carcinomas of the Head and Neck,” Am J Pathol, 151(2):499-508 (1997); and Uria J. A., et al., “Regulation of Collagenase-3 Expression in Human Breast Carcinomas is Mediated by Stromal-Epithelial Cell Interactions,” Cancer Res, 57(21):4882-8 (1997); and Airola K., et al., “Human Collagenase-3 is Expressed in Malignant Squamous Epithelium of the Skin,” J Invest Dermatol, 109:225-231 (1997); and Freije J. M., et al., “Molecular Cloning and Expression of Collagenase-3, A Novel Human Matrix Metalloproteinase Produced by Breast Carcinomas,” J Biol Chem, 269(24):16766-73 (1994); and Uria J. A., et al., “Regulation of Collagenase-3 Expression in Human Breast Carcinomas is Mediated by Stromal-Epithelial Cell Interactions,” Cancer Res, 57(2):4882-8 (1997); and their contents are hereby incorporated by reference in their entirety.

In preferred embodiments (Eneo87 and Eneo88) E has the following structure:

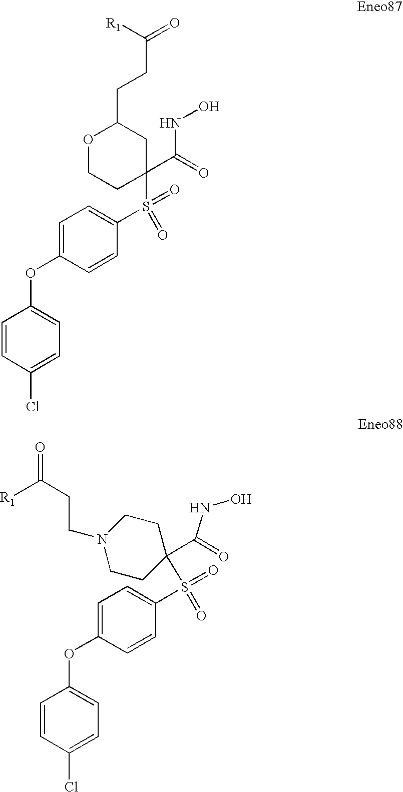

wherein R₁ is as shown below:

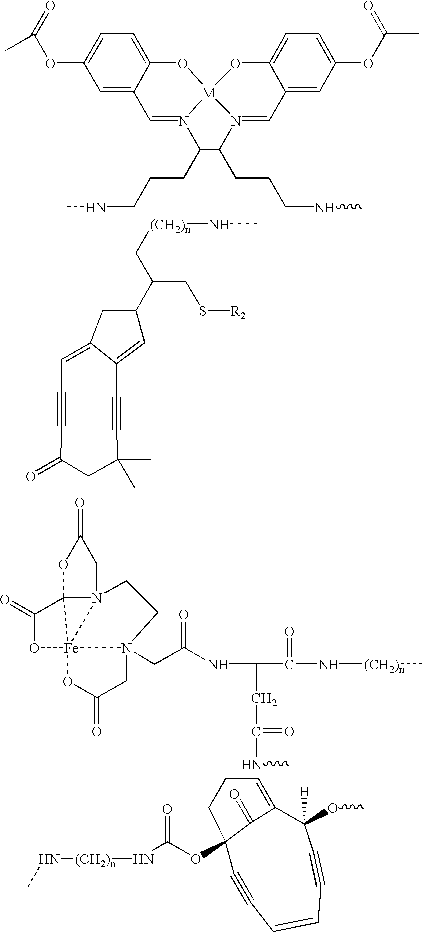

and wherein the dotted line is the site of attachment to the MMP ligand the wavy line is the site of attachment to the remainder of the targeted drug; and wherein n=1,2,3,4,5,6 or about 6, and M is Cu(II) of Fe(II); and wherein R₂ is an acyl group, or R₂ is a clock-like time delay trigger, or a bioreversible thiol protecting group such as —S—R₃ where R₃ is any group such that the disulfide is reduced to the thiol by cells.

Targeted Delivery of Activators of Innate Immunity

Evolution has endowed the body with the ability to mount effective and almost immediate nonspecific defenses against infectious agents. The body is highly tuned to detect and react to molecules derived from pathogens. The result is a rapid and massive influx of inflammatory cells such as neutrophils, monocyes, macrophages, natural killer cells and delta/gamma T cells. The release of a number of inflammatory cytokines amplifies the response. Phagocytosis and the production of toxic radicals such as superoxide, hypochlorous acid, nitric oxide, and peroxynitrite contribute to the killing of the invading microorganisms. The same intense immune response that is ellicited by microorganisms can be directed against tumors by selective targeting of activators of innate immunity to tumors. The ability of activated neutrophils, macrophages, monocytes and NK cells to kill tumors is well documented in numerous models. A pronounced synergy is expected when tumors are simultaneously targeted with both activators of innate immunity and antigen receptor specific T cell mediated immunity. The immune system evolved to deal precisely with this situation. Both innate and adaptive immune responses are simultaneously triggered by infectious agents and mutually reinforce and amplify the net immune response.

The following references relate to this matter: Seino K., et al., “Antitumor Effect of Locally Produced CD95 Ligand,” Nat Med, 3(2):165-70 (1997); and Shimizu M., et al., “Induction of Antitumor Immunity with Fas/APO-1 Ligand (CD95L)-Transfected Neuroblastoma Neuro-2a Cells,” J Immunol, 162(12):7350-7 (1999); and Stoppacciaro A., et al., “Regression of an Established Tumor Genetically Modified to Release Granulocyte Colony-stimulating Factor Requires Granulocyte-T Cell Cooperation and T Cell-produced Interferon γ,” J Exp Med, 178:151-161 (1993); and Cavallo F., et al., “Role of Neutrophils and CD4⁺ T Lymphocytes in the Primary and Memory Response to Nonimmunogenic Murine Mammary Adenocarcinoma made Immunogenic by IL-2 Gene,” J Immunol, 149(11):3627-3635 (1992); and Griffith T. S., et al., “Monocyte-mediated Tumoricidal Activity via the Tumor Necrosis Factor-related Cytokine, TRAIL,” J Exp Med, 189(8):1343-1353 (1999); and Yoneda Y.; Yoshida R., “The Role of T Cells in Allografted Tumor Rejection: IFN-γ Released from T Cells is Essential for Induction of Effector Macrophages in the Rejection Site,” J Immunol, 160:6012-6017 (1998); and Noffz G. et al., “Neutrophils but not Eosinophils are Involved in Growth Suppression of IL-4-Secreting Tumors,” J Immunol, 160:345-350 (1998); and Gerrard T. L., et al., “Human Neutrophil-Mediated Cytotoxicity to Tumor Cells,” JNCI, 66(3):483-488 (1981); and Clark R. A.; Klebanoff S. J., “Role of the Myeloperoxidase-H₂O₂-Halide System in Concanavalin A-Induced Tumor Cell Killing by Human Neutrophils,” J Immunol, 122(6):2605-2610 (1979); and Hafeman D. G.; Lucas Z. J., “Polymorphonuclear Leukocyte-Mediated, Antibody-Dependent, Cellular Cytotoxicity against Tumor Cells: Dependence on Oxygen and the Respiratory Burst,” J Immunol, 123(1):55-62 (1979); and Clark R. A.; Szot S., “The Myeloperoxidase-Hydrogen Peroxide-Halide System as Effector of Neutrophil-Mediated Tumor Cell Cytotoxicity,” J Immunol, 126(4):1295-1301 (1981); and Clark R. A.; Klebanoff S. J., “Neutrophil-mediated Tumor Cell Cytotoxicity: Role of the Peroxidase System,” J Exp Med, 141:1442-1447 (1975); and Pericle F., et al., “CD44 is a Cytotoxic Triggering Molecule on Human Polymorphonuclear Cells,” J Immunol, 157:4657-4663 (1996); and their contents are hereby incorporated by reference in their entirety.

A variety of mechanisms may be employed to target the innate immune system against tumors. Fundamentally this approach involves delivering, selectively to the tumor key, signal molecules that trick the immune system into regarding the tumor as a pathogenic microorgansim. A major advantage of this approach is that it is not necessary to sensitize or immunize the patient to evoke the immune response. Potent signal molecules that can be delivered to tumors to stimulate the innate immune system include:

1.) N-formyl peptide receptor agonists

2.) Tuftsin receptor agonists

3.) Lipoxin A(4) receptor agonists

4.) Leukotriene B4 agonists

5.) 3-formyl-1 -butyl-pyrophosphates receptor agonists

6.) Gal alpha(1,3)Gal. analogs

It is important that the targeting specificity of the drug be defined by the targeting ligands not by the interaction of the immune stimulator with immune effector cells. This can be accomplished by employing masked immunostimulator ligands, that are unmasked by a trigger after localization to tumor cells has occurred.

Targeted Delivery of Ligands for the Formyl Peptide Receptor

The formyl peptide receptor(s) (FPR) is a protein present on the surface of neutrophils, monocytes and macrophages that bind to n-formyl peptides with high affinity. Bacteria initiate protein translation with n-formyl methionine and the innate immune system has evolved to recognize the presence of n-formyl methionine peptides as a sign of bacterial infection. A large number of small formyl peptides such as N-formyl-Met-Leu-Phe are potent chemotactic and activating agents for leukocytes. Superoxide generation, and the release of inflammatory cytokines are potently stimulated by activation of FPR receptors.

Antibodies coupled to ligands for FPR have been explored as antitumor agents, but failed to show significant efficacy in vivo.

It is known that linkers with fluorescent groups may be attached to the carboxy terminus of N-formyl-Met-Leu-Phe without impairing affinity or biological activity, while the formyl group is critical for effective binding and activity for most, but not all analogs. For example, N-formyl-methionyl-norleucyl-leucyl-phenylalanine-phenylalanine and N-acetyl-methionyl-norleucyl-leucyl-phenylalanine-phenylalanine are both extremely potent activactors of FPR. The N unsubstituted analog is less potent, but still active at nanomolar concentrations. Certain N-terminal carbamates are also extremely potent activators of FPR.

The following reference relate to this matter: Obrist R., et al., “Conjugation Behaviour of Different Monoclonal Antibodies to F-Methionyl-Leucyl-Phenylalanine,” Int J Immunopharmacol, 8(6):629-32 (1986); and Yuli I.; Snyderman R., “Extensive Hydrolysis of N-Formyl-L-Methionyl-L-Leucyl-L-[3H] Phenylalanine by Human Polymorphonuclear Leukocytes. A Potential Mechanism for Modulation of the Chemoattractant Signal,” J Biol Chem, 261(11):4902-8 (1986); and Bycroft B. W., et al., “Antibacterial and Immunostimulatory Properties of Chemotactic N-Formyl Peptide Conjugates of Ampicillin and Amoxicillin,” Antimicrob Agents Chemother, 33(9):1516-21 (1989); and Obrist R., et al., “Chemotactic Monoclonal Antibody Conjugates: A Comparison of Four Different F-Met-Peptide-Conjugates,” Biochem Biophys Res Comm, 155(3):1139-44 (1988); and Niedel J., et al., “Covalent Affinity Labeling of the Formyl Peptide Chemotactic Receptor,” J Biol Chem, 255(15):7063-6 (1980); and Marasco W. A., et al., “Covalent Affinity Labeling, Detergent Solubilization, and Fluid-Phase Characterization of the Rabbit Neutrophil Formyl Peptide Chemotaxis Receptor,” Biochemistry, 24(9):2227-36 (1985); and Niedel J., “Detergent Solubilization of the Formyl Peptide Chemotactic Receptor. Strategy Based on Covalent Affinity Labeling,” J Biol Chem, 256(17):9295-9 (1981); and Vilven J. C., et al., “Strategies for Positioning Fluorescent Probes and Crosslinkers on Formyl Peptide Ligands,” J Recept Signal Transduct Res, 18(2-3):187-221 (1998); and Rot A., et al., “A Series of Six Ligands for the Human Formyl Peptide Receptor: Tetrapeptides with High Chemotactic Potency and Efficacy,” Proc Natl Acad Sci USA, 84(22):7967-71 (1987); and Mills J. S., et al., “Identification of a Ligand Binding Site in the Human Neutrophil Formyl Peptide Receptor Using a Site-Specific Fluorescent Photoaffinity Label and Mass Spectrometry,” J Biol Chem, 273(17):10428-35 (1998); and Prossnitz E. R.; Ye R. D. “The N-Formyl Peptide Receptor: A Model for the Study of Chemoattractant Receptor Structure and Function,” Pharmacol Ther, 74(1):73-102 (1997); and Fay S. P., et al., “Multiparameter Flow Cytometric Analysis of a pH Sensitive Formyl Peptide with Application to Receptor Structure and Processing Kinetics,” Cytometry, 15(2):148-53 (1994); and Johansson B., et al., “N-Formyl Peptide Receptors in Human Neutrophils Display Distinct Membrane Distribution and Lateral Mobility when Labeled with Agonist and Antagonist,” J Cell Biol, 121(6):1281-9 (1993); and Painter R. G., et al., “Photoaffinity Labeling of the N-Formyl Peptide Receptor of Human Polymorphonuclear Leukocytes,” J Cell Biochem, 20(2):203-14 (1982); and Allen R. A., et al., “Physicochemical Properties of the N-Formyl Peptide Receptor on Human Neutrophils,” J Biol Chem, 261(4):1854-7 (1986); and Allen R. A., et al., “Preparation and Properties of an Improved Photoaffinity Ligand for the N-Formyl Peptide Receptor,” Biochim Biophys Acta, 882(3):271-80 (1986); and Niedel J. E., et al., “Receptor-Mediated Internalization of Fluorescent Chemotactic Peptide by Human Neutrophils,” Science, 205(4413):1412-4 (1979). Obrist R., et al., “Acute and Subacute Toxicity of Chemotactic Conjugates between Monoclonal Antibody and fMet-Leu-Phe in Humans: A Phase I Clinical Trial,” Cancer Immunol Immunother, 32(6):406-8 (1991); and Obrist R.; Sandberg A. L., “Enhancement of Macrophage Invasion of Tumors by Administration of Chemotactic Factor-Antitumor Antibody Conjugates,” Cell Immunol, 81(1):169-74 (1983); and Balazovich K. J., et al., “Tumor Necrosis Factor-Alpha and FMLP Receptors are Functionally Linked During FMLP-Stimulated Activation of Adherent Human Neutrophils,” Blood, 88(2):690-6 (1996); and Freer R. J., et al., “Further Studies on the Structural Requirements for Synthetic Peptide Chemoattractants,” Biochemistry, 19:2404-2410 (1980); and Gao J. L., et al., “A High Potency Nonformylated Peptide Agonist for the Phagocyte N-Formylpeptide Chemotactic Receptor,” J Exp Med, 180:2191-2197 (1994); and Higgins J. D., et al., “N-Terminus Urea-Substituted Chemotactic Peptides: New Potent Agonists and Antagonists toward the Neutrophil fMLF Receptor,” J Med Chem, 39(5):1013-1015 (1996) and their contents are hereby incorporated by reference in their entirety.

In a preferred embodiment, E is comprised of a masked ligand for FPR that is masked in a bioreversible manner. E may be configured either to tether an FPR ligand to the target or to release an FPR ligand in the microenvironment of the target.

In preferred embodiment (Einl-Ein3), of E is comprised of the structure shown below:

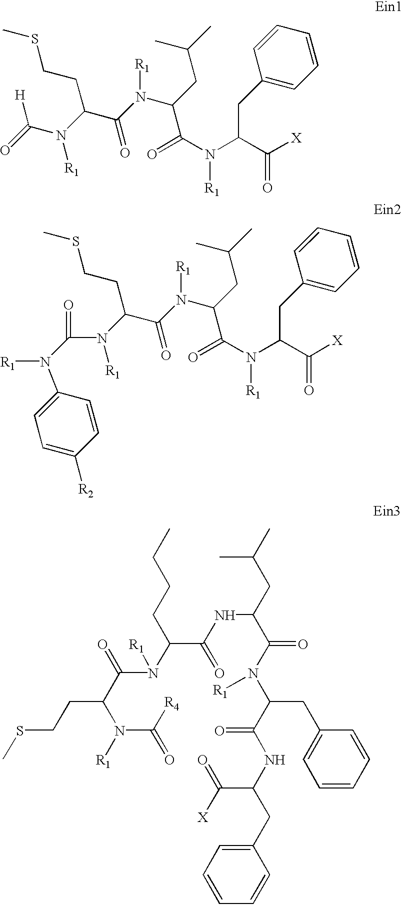

wherein X is either OH or the site of linker attachment to the remainder of the target drug; and wherein R₁ is either H, or a bioreversible protecting group, or masking trigger; and R₂ is Cl, methyl, or methoxy; and R₄ is H, or methyl; and wherein either X or R₁ has a site of attachment to the remainder of ET. A large number of suitable triggers are described in the trigger section of this document.

In a preferred embodiment (Ein4), E is comprised of the following structure:

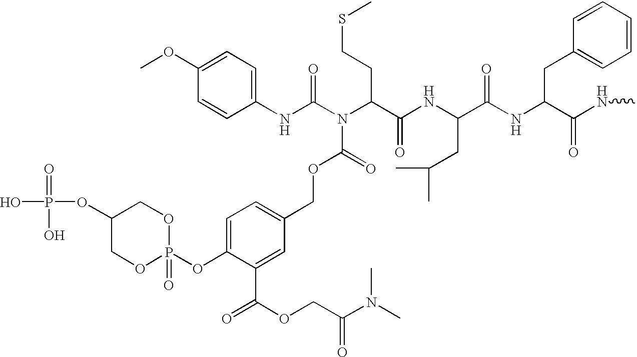

wherein the wavy line is the site of attachment to the remainder of the targeted drug. Activation of the trigger by esterase will liberate the biologically active FPR receptor activator.

Targeted Delivery of Tuftsin Analogs

Tuftsin is the tetrapeptide threonyl-lysyl-prolyl-arginine. Tuftsin is a potent activator of granulocyte, macrophage and monocyte function. Phagocytosis, chemotaxis, hydrogen peroxide and superoxide production, and tumor necrosis factor production are all stimulated by tuftsin. NK cell activity is also markedly potentiated by tuftsin. Tuftsin exerts considerable antitumor activity in a number of animal models. A large number of tufsin analogs, which bind to the tuftsin receptor, and evoke potent activity are known. Fluorescent analogs which retain activity have been synthesized by derivatizing the C terminus of tuftsin. The following references relate to this matter: Najjar V. A.; Fridkin M., “Anitneoplastic, Immunogenic and Other Effects of the Tetrapeptide Tuftsin: A Natural Macrophage Activator,” Ann of New York Acad Sci, 419:1-273 (1983); and Nishioka K., et al., “Antitumor Effect of Tuftsin,” Mol Cell Biochem, 41:13-8 (1981); and Fridkin M.; Najjar V. A., “Tuftsin: Its Chemistry, Biology, and Clinical Potential,” Crit Rev Biochem Mol Biol, 24(1):1-40 (1989); and Bar-Shavit Z., et al., “Functional Tuftsin Binding Sites on Macrophage-Like Tumor Line P388D1 and on Bone Marrow Cells Differentiated in Vitro into Mononuclear Phagocytes,” Mol Cell Biochem, 30(3):151-5 (1980); and Verdini A. S., et al., “Immunostimulation by a Partially Modified Retro-Inverso-Tuftsin Analogue Containing Thr1 psi[NHCO](R,S)Lys2 Modification,” J Med Chem, 34(12):3372-9 (1991); and Florentin I., et al., “In Vivo Immunopharmacological Properties of Tuftsin (Thr-Lys-Pro-Arg) and Some Analogues,” Methods Find Exp Clin Pharmacol, 8(2):73-80 (1986); and Kraus-Berthier L., et al., “In Vivo Immunopharmacological Properties of Tuftsin and Four Analogs,” Immunopharmacology, 25(3):261-7 (1993); and Phillips J. H., et al., “Tuftsin: a Naturally Occurring Immunopotentiating Factor. I. In Vitro Enhancement of Murine Natural Cell-Mediated Cytotoxicity,” J Immunol, 126(3):915-21 (1981); and Siemion I Z, Kluczyk A., “Tuftsin: on the 30-Year Anniversary of Victor Najjar's Discovery,” Peptides, 20(5):645-74 (1999); and Gottlieb P., et al., “Receptor-Mediated Endocytosis of Tuftsin by Macrophage Cells,” Biochem Biophys Res Commun, 119(1):203-11 (1984); and Dagan S., et al., “Tuftsin Analogues: Synthesis, Structure-Function Relationships, and Implications for Specificity of Tuftsin's Bioactivity,” J Med Chem, 29(10):1961-8 (1986); and Bump N. J., et al., “The characteristics of Purified HL60 Tuftsin Receptors,” Mol Cell Biochem, 92(1):77-84 (1990); and Cillari E., et al., “The Macrophage-Activating Tetrapeptide Tuftsin Induces Nitric Oxide Synthesis and Stimulates Murine Macrophages to Kill Leishmania Parasites In Vitro,” Infect Immun, 62(6):2649-52 (1994); and Tzehoval E., et al., “Tuftsin (an Ig-associated Tetrapeptide) Triggers the Immunogenic Function of Macrophages: Implications for Activation of Programmed Cells,” Proc Natl Acad Sci USA, 75(7):3400-4 (1978); and Bar-Shavit Z., et al., “Tuftsin-Macrophage Interaction: Specific Binding and Augmentation of Phagocytosis,” J Cell Physiol, 100(1):55-62 (1979); and the contents are hereby incorporated by reference in their entirety.

In a preferred embodiment, E is comprised of a masked tufsin receptor activator that is masked in a bioreversible fashion. E may be configured either to a tuftsin receptor agonist to the target or to release it in the microenvironment of the target. In a preferred embodiment (Ein5), E has the structure:

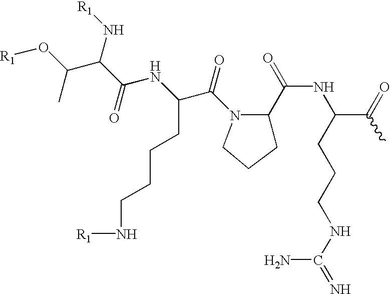

wherein the wavy line is the site of attachment of the remainder of the targeted drug and R₁ is H, or a masking trigger which when activated generates the biologically active tuftsin agonist. In a preferred embodiment (Ein6), E has the following structure:

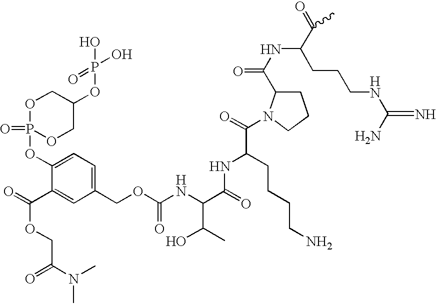

Activation of the clock-like time delayed masking trigger by esterase will liberate the biologically active Tuftsin receptor agonist.

Targeted Delivery of Lipoxin A4 Receptor Activators

The oligopeptide Trp-Lys-Tyr-Met-Val-D-Met-NH2 is an extremely potent chemotactic agent, which activates neutrophils and monocytes to produce hydrogen peroxide and superoxide, and release inflammatory cytokines. This activity is mediated by binding to the lipoxin A4 receptor at picomolar concentrations. At nanomolar concentration the FPR is also activated. The following references relate to this matter: Seo J. K., et al., “A Peptide with Unique Receptor Specificity: Stimulation of Phosphoinositide Hydrolysis and Induction of Superoxide Generation in Human Neutrophils,” J Immunol, 158(4):1895-901 (1997); and Bae Y. S., et al., “Trp-Lys-Tyr-Met-Val-D-Met is a Chemoattractant for Human Phagocytic Cells,” J Leukoc Biol, 66(6):915-22 (1999); and Bae Y. S., et al., “Trp-Lys-Tyr-Met-Val-D-Met Stimulates Superoxide Generation and Killing of Staphylococcus Aureus via Phospholipase D Activation in Human Monocytes,” J Leukoc Biol, 65(2):241-8 (1999); and Dahlgren C., et al., “The Synthetic Chemoattractant Trp-Lys-Tyr-Met-Val-Dmet Activates Neutrophils Preferentially through the Lipoxin A(4) Receptor,” Blood, 95(5):1810-8 (2000); and Le Y., et al., “Utilization of Two Seven-Transmembrane, G Protein-Coupled Receptors, Formyl Peptide Receptor-Like 1 and Formyl Peptide Receptor, by the Synthetic Hexapeptide WKYMVm for Human Phagocyte Activation.” J Immunol, 163(12):6777-84 (1999); and Seo J. K., et al., “Distribution of the Receptor for a Novel Peptide Stimulating Phosphoinositide Hydrolysis in Human Leukocytes,” Clin Biochem, 31(3):137-41 (1998); and the contents are hereby incorporated by reference in their entirety.

In a preferred embodiment E is an actiivator of the lipoxin A4 receptor. In a preferred embodiment (Ein7), E is comprised of the following structure structure:

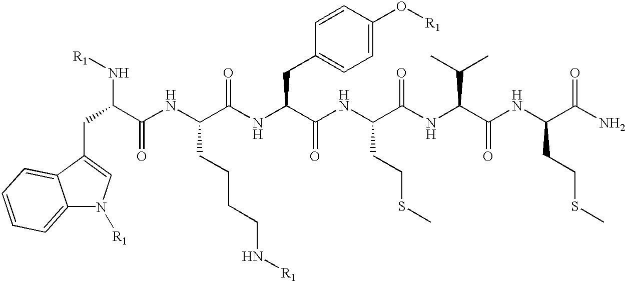

wherein the Met is the D isomer and R₁ is H or a trigger which when activated generates the biologically active lipoxin A4 receptor agonist; and wherein R₁ bears a site of attachment to the remainder of the targeted drug. In a preferred embodiment (Ein8), E is comprised of the following structure:

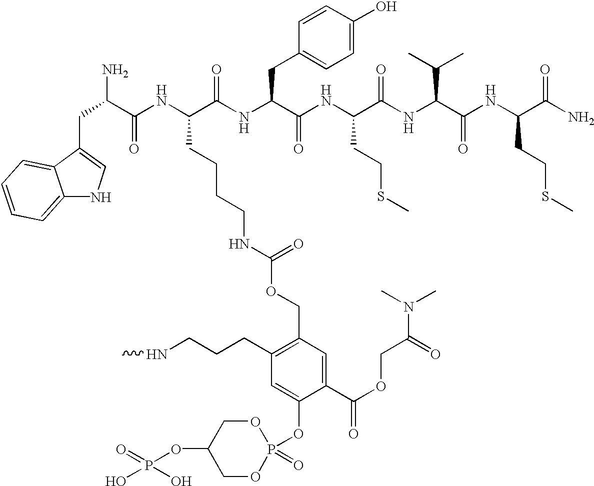

wherein the wavy line is the site of linker attachment to the remainder of ET. Esterase can activate the clock-like time delayed masking trigger which will free the lipoxin A4 agonist from the targeted drug complex.

Targeted Leukotriene B4 Agonists

Leukotriene B is a potent inflammatory mediator with chemotactic and neutrophil/monocyte activating properties. Neutrophil degranulation, superoxide production and vascular permeability are all markedky increased by leukotriene B4. Leukotriene B production is dramatically increased by phospholipase A2. Phospholipase A2 activators have been reported to induce massive inflammation in gliomas and produce tumor regression in animals. Because of its importance in inflammation, extensive research has focused on the development of antagonists for leukotriene B4. In the course of these studies, a large number of extremely potent leukotriene B4 agonists have been discovered. The following references relate to this matter: Soyombo O., et al., “Structure/Activity Relationship of Leukotriene B4 and its Structural Analogues in Chemotactic, Lysosomal-Enzyme Release and Receptor-Binding Assays,” Eur J Biochem, 218(1):59-66 (1993); and Leblanc Y., et al., “Analogs of Leukotriene B4: Effects of Modification of the Hydroxyl Groups on Leukocyte Aggregation and Binding to Leukocyte Leukotriene B4 Receptors,” Prostaglandins, 33(5):617-25 (1987); and Gapinski D. M., et al., “Benzophenone Dicarboxylic Acid Antagonists of Leukotriene B4. 2. Structure-Activity Relationships of the Lipophilic Side Chain,” J Med Chem, 33(10):2807-13 (1990); and Bomalaski J. S.; Mong S., “Binding of Leukotriene B4 and its Analogs to Human Polymorphonuclear Leukocyte Membrane Receptors,” Prostaglandins, 33(6):855-67 (1987); and Jackson W. T., et al., “Design, Synthesis, and Pharmacological Evaluation of Potent Xanthone Dicarboxylic Acid Leukotriene B4 Receptor Antagonists,” J Med Chem, 36(12):1726-34 (1993); and Hoover R. L., et al., “Leukotriene B4 Action on Endothelium Mediates Augmented Neutrophil/Endothelial Adhesion,” Proc Natl Acad Sci USA, 81(7):2191-3 (1984); and Palmblad J., et al., “Leukotriene B4 Triggers Highly Characteristic and Specific Functional Responses in Neutrophils: Studies of Stimulus Specific Mechanisms,” Biochim Biophys Acta, 871(1):92-102 (1988); and Crooks S. W.; Stockley R. A., “Leukotriene B4,” Int J Biochem Cell Biol, 30(2):173-8 (1998); and Lam B. K., et al., “Phospholipase A2 as Leukotriene B4 Secretagogue for Human Polymorphonuclear Leukocytes,” Adv Exp Med Biol, 275:183-91 (1990); and Goddard D. H., et al., “Phospholipase A2-Mediated Inflammation Induces Regression of Malignant Gliomas,” Cancer Lett, 102(1-2):1-6 (1996); and Daines R. A., et al., “Trisubstituted Pyridine Leukotriene B4 Receptor Antagonists: Synthesis and Structure-Activity Relationships,” J Med Chem, 36(22):3321-32 (1993); and Poudrel J. M., et al., “Synthesis and Structure-Activity Relationships of New 1,3-Disubstituted Cyclohexanes as Structurally Rigid Leukotriene B₄ Receptor Antagonists,” J Med Chem, 42(26):5289-5310 (1999); and Kingsbury W. D., et al., “Synthesis of Structural Analogs of Leukotriene B4 and their Receptor Binding Activity,” J Med Chem, 36(22):3308-20 (1993); and Konno M., et al., “Synthesis of Structural Analogues of Leukotriene B4 and their Receptor Binding Activity,” Bioorg Med Chem, 5(8):1621-47 (1997); and the contents are hereby incorporated by reference in their entirety.

In a preferred embodiment, E is comprised of a leukotriene B4 agonist. In a preferred embodiment (Ein9), E is comprised of the following structure:

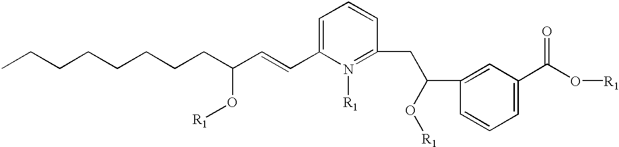

wherein R₁ is H, or the site of attachment to the remainder of the targeted drug.

In a preferred embodiment (Ein10), E has the following structure:

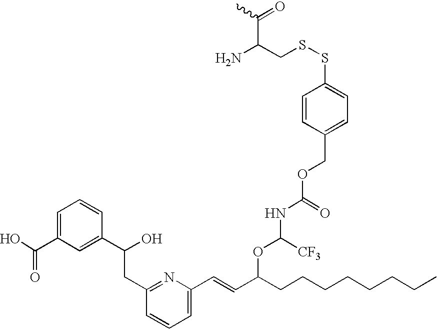

wherein the wavy line is the site of attachment to the remainder of the drug complex. Cleavage of the disulfide bond will free the leukotriene B4 agonist.

Targeted Delivery of γ/δ T Cell Activators

γ/δ T cells are a class of lymphocytes, which recognize antigens in a manner analogous to antibodies in the absence of MHC restriction. γ/δ T cells have been implicated in immunity to tuberculosis, malaria, listeria, and herpes simplex virus. Contact hypersensitivity, autoimmunity, graft versus host disease, and tumor rejection have all been associated with γ/δ T cells. γ/δ T cells produce target damage by perform mediated cytotoxicity, and the release of a variety of cytokines such as interferon gamma, macrophage inflammatory protein, lymphotactin, RANTES, and tumour necrosis factor alpha.

A high percentage of human γ/δ T cells are activated by phosphoantigens derived from mycobacterium such as prenyl pyrophosphate analogs. 3-Formyl-1-butyl-pyrophosphate and related derivatives are extremely potent activators of γ/δ T cells. The following references relate to this matter: Belmant C, et al., “3-Formyl-1-butyl Pyrophosphate a Novel Mycobacterial Metabolite-Activating Human Gammadelta T Cells,” J Biol Chem, 274(45):32079-84 (1999); and Huber H., et al., “Activation of Murine Epidermal TCR-Gamma Delta+ T Cells by Keratinocytes Treated with Contact Sensitizers,” J Immunol, 155(6):2888-94 (1995); and Groh V., et al., “Broad Tumor-Associated Expression and Receognition by Tumor-Derived γδ T Cells of MICA and MICB,” PNAS, 96(12):6879-6884 (1999); and Sciammas R., et al., “T Cell Receptor-Gamma/Delta Cells Protect Mice from Herpes Simplex Virus Type 1-Induced Lethal Encephalitis,” J Exp Med, 185(11):1969-75 (1997); and Bukowski J. F., et al., “Crucial Role of TCR Gamma Chain Junctional Region in Prenyl Pyrophosphate Antigen Recognition by Gamma Delta T Cells,” J Immunol, 161(1):286-93 (1998); and Zocchi M. R., et al., “Selective Lysis of the Autologous Tumor by Delta TCS1+ Gamma/Delta+ Tumor-Infiltrating Lymphocytes from Human Lung Carcinomas,” Eur J Immunol, 20(12):2685-9 (1990); and Morita C. T., et al., “Direct Presentation of Nonpeptide Prenyl Pyrophosphate Antigens to Human Gamma Delta T Cells,” Immunity, 3(4):495-507 (1995); and Elloso M. M., et al., “The Effects of Interleukin-15 on Human Gammadelta T Cell Responses to Plasmodium Falciparum in Vitro,” Immunol Lett, 64(2-3):125-32 (1998); and Ferrarini M., et al., “Killing of Laminin Receptor-Positive Human Lung Cancers by Tumor Infiltrating Lymphocytes Bearing Gammadelta(+) T-Cell Receptors,” J Natl Cancer Inst, 88(7):436-41 (1996); and Maeurer M. J., et al., “Human Intestinal Vdelta1+ Lymphocytes Recognize Tumor Cells of Epithelial Origin,” J Exp Med, 183(4):1681-96 (1996); and Yu S., et al., “Expansion and Immunological Study of Human Tumor Infiltrating Gamma/Delta T Lymphocytes In Vitro,” Int Arch Allergy Immunol, 119(1):31-7 (1999); and Weintraub B. C., et al., “Gamma Delta T Cells can Recognize Nonclassical MHC in the Absence of Conventional Antigenic Peptides,” J Immunol, 153(7):3051-8 (1994); and Kabelitz D, et al. “Gamma Delta T Cells, their T Cell Receptor Usage and Role in Human Diseases,” Springer Semin Immunopathol, 21(1):55-75 (1999); and Cipriani B., et al., “Activation of C-C Beta-Chemokines in Human Peripheral Blood Gammadelta T Cells by Isopentenyl Pyrophosphate and Regulation by Cytokines,” Blood, 95(1):39-47 (2000); and Bukowski J. F., et al., “Human Gamma Delta T Cells Recognize Alkylamines Derived from Microbes, Edible Plants, and Tea: Implications for Innate Immunity,” Immunity, 11 (1):57-65 (1999); and Laad A. D, et al., “Human Gamma Delta T Cells Recognize Heat Shock Protein-60 on Oral Tumor Cells,” Int J Cancer, 80(5):709-14 (1999); and Burk M. R., et al., “Human V Gamma 9-V Delta 2 Cells are Stimulated in a Cross-Reactive Fashion by a Variety of Phosphorylated Metabolites,” Eur J Immunol, 25(7):2052-8 (1995); and Garcia V. E., et al., “IL-15 Enhances the Response of Human Gamma Delta T Cells to Nonpeptide [Correction of Nonpetide] Microbial Antigens,” J Immunol, 160(9):4322-9 (1998); and Chandler P., et al., “Immune Responsiveness in Mutant Mice Lacking T-Cell Receptor Alpha Beta+ Cells,” Immunology, 85(4):531-7 (1995); and Dechanet J., et al., “Implication of Gammadelta T Cells in the Human Immune Response to Cytomegalovirus,” J Clin Invest, 103:10):1437-49 (1999); and Wei Y., et al., “Induction of Autologous Tumor Killing by Heat Treatment of Fresh Human Tumor Cells: Involvement of Gamma Delta T Cells and Heat Shock Protein 70,” Cancer Res, 56(5):1104-10 (1996); and Blazar B. R., et al., “Lethal Murine Graft-Versus-Host Disease Induced by Donor Gamma/Delta Expressing T Cells with Specificity for Host Nonclassical Major Histocompatibility Complex Class Ib Antigens,” Blood, 87(2):827-37 (1996); and Bialasiewicz A. A., et al., “Alpha/Beta- and Gamma/Delta TCR(+) Lymphocyte Infiltration in Necrotising Choroidal Melanomas,” Br J Ophthalmol, 83(9):1069-73 (1999); and Gan Y. H.; Malkovsky M., “Mechanisms of Simian Gamma Delta T Cell Cytotoxicity against Tumor and Immunodeficiency Virus-Infected Cells,” Immunol Lett, 49(3): 191-6 (1996) Manfredi A. A., et al., “Mycobacterium Tuberculosis Exploits the CD95/CD95 Ligand System of Gammadelta T Cells to Cause Apoptosis,” Eur J Immunol, 28(6):1798-806 (1998); and Tanaka Y., et al., “Natural and Synthetic Non-Peptide Antigens Recognized by Human Gamma Delta T Cells,” Nature, 375(6527):155-8 (1995); and Tanaka Y., et al., “Nonpeptide Ligands for Human Gamma Delta T Cells,” Proc Natl Acad Sci USA, 91(17):8175-9 (1994); and Poccia F., et al., “Phosphoantigen-Reactive Vgamma9Vdelta2 T Lymphocytes Suppress in Vitro Human Immunodeficiency Virus Type 1 Replication by Cell-Released Antiviral Factors including CC Chemokines,” J Infect Dis, 180(3):858-61 (1999); and De Libero G., et al., “Selection by Two Powerful Antigens may Account for the Presence of the Major Population of Human Peripheral Gamma/Delta T Cells,” J Exp Med, 173(6):1311-22 (1991); and Boullier S., et al., “Regulation by Cytokines (IL-12, IL-15, IL-4 and IL-10) of the Vgamma9Vdelta2 T Cell Response to Mycobacterial Phosphoantigens in Responder and Anergic HIV-Infected Persons,” Eur J Immunol, 29(1):90-9 (1999); and Ferrini S., et al., “Retargeting of T-Cell-Receptor Gamma/Delta+ Lymphocytes against Tumor Cells by Bispecific Monoclonal Antibodies. Induction of Cytolytic Activity and Lymphokine Production,” Int J Cancer Suppl, 4:53-5 (1989); and Salerno A., et al., “Role of Gamma Delta T Lymphocytes in Immune Response in Humans and Mice,” Crit Rev Immunol, 18(4):327-57 (1998); and Choudhary A., et al., “Selective Lysis of Autologous Tumor Cells by Recurrent Gamma Delta Tumor-Infiltrating Lymphocytes from Renal Carcinoma,” J Immunol, 154(8):3932-40 (1995); and Catalfamo M., et al., “Self-Reactive Cytotoxic Gamma Delta T Lymphocytes in Graves′ Disease Specifically Recognize Thyroid Epithelial Cells,” J Immunol, 156(2):804-11 (1996); and Constant P., et al., “Stimulation of Human Gamma Delta T Cells by Nonpeptidic Mycobacterial Ligands,” Science, 264(5156):267-70 (1994); and Li H., et al., “Structure of the Vdelta Domain of a Human Gammadelta T-Cell Antigen Receptor,” Nature, 391(6666):502-6 (1998); and Zhao X., et al., “Accumulation of Gamma/Delta T Cells in Human Dysgerminoma and Seminoma: Roles in Autologous Tumor Killing and Granuloma Formation,” Immunol Invest, 24(4):607-18 (1995); and Cipriani B., et al., “Activation of C-C-β-Chemokines in Human Peripheral Blood γδ T Cells by Isopentenyl Pyrophosphate and Regulation by Cytokines,” Blood, 95(1):39-47 (2000); and Bialasiewicz A. A., et al., “Alpha/Beta- and Gamma/Delta TCR(+) Lymphocyte Infiltration in Necrotising Choroidal Melanomas,” Br J Ophthalmol, 83(9):1069-73 (1999); and Yin Z., et al., “Dominance of IL-12 over IL-4 in Gamma Delta T Cell Differentiation Leads to Default Production of IFN-Gamma: Failure to Down-Regulate IL-12 Receptor Beta 2-Chain Expression,” J Immunol, 164(6):3056-64 (2000); and Thomas M. L., et al., “Gammadelta T Cells Lyse Autologous and Allogenic Oesophageal Tumours: Involvement of Heat-Shock Proteins in the Tumour Cell Lysis,” Cancer Immunol Immunother, 48(11):653-9 (2000); and Fujimiya Y., et al., “In Vitro Interleukin 12 Activation of Peripheral Blood CD3(+)CD56(+) and CD3(+)CD56(−) Gammadelta T Cells from Glioblastoma Patients,” Clin Cancer Res, 3(4):633-43 (1997); and Yamaguchi T., et al., “Interleukin-15 Effectively Potentiates the in Vitro Tumor-Specific Activity and Proliferation of Peripheral Blood Gammadelta T Cells Isolated from Glioblastoma,” Cancer Immunol lmmunother, 47(2):97-103 (1998); and Poccia F., et al., “Phosphoantigen-Reactive Vgamma9Vdelta2 T Lymphocytes Suppress in Vitro Human Immunodeficiency Virus Type 1 Replication by Cell-Released Antiviral Factors Including CC Chemokines,” J Infect Dis, 180(3):858-61 (1999); and Wesch D., et al., “Comparative Analysis of Alpha Beta and Gamma Delta T Cell Activation by Mycobacterium Tuberculosis and Isopentenyl Pyrophosphate,” Eur J Immunol, 27(4):952-6 (1997); and the contents are hereby incorporated by reference in their entirety.

In a preferred embodiment, E is an activator of γ/δ T cells which is masked in a bioreversible manner.

In preferred embodiments (Ein11 and Ein12), E is comprised of the following structures:

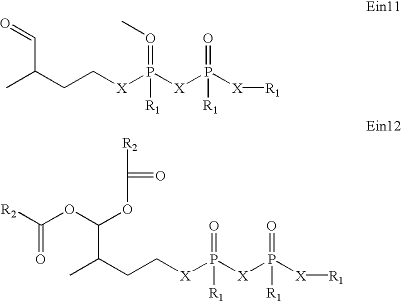

wherein X is O, or CH₂, and R₁ is OH, a bioreversible masking group, or a site of attachment to the remainder of the targeted drug, and R₂ is a lower alkyl group, or a phenyl group, or other group such that the resulting ester is cleaved by esterase; and wherein R₂ may also bear a site of attachment to the remainder of ET.

In a preferred embodiment (Ein13), E is comprised of the following structure:

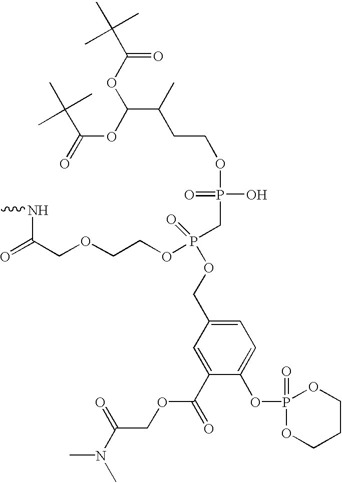

In this embodiment, the active formyl analog is generated following cleavage of the pivalate by esterase and following triggering of the clock-like time delayed trigger by esterase.

In a preferred embodiment, E is comprised of two masked activators of γ/δ T cells, which are masked in a bioreversible manner, connected by a linker, which is connencted to the remainder of the targeted drug; wherein the linker is selected so as to allow bivalent binding to the γ/δ T cell of the unmasked formyl pyro phosphate ligands.

Targeted Delivery of alpha-Galactosyl Epitopes

Humans naturally produce high titre antibodies to terminal α-galactosyl-(1,3)-β-galactosyl structures. These antibodies mediate the hyperacute rejection of xenographs. In addition, NK cells recognize terminal α-galactosyl-(1,3)Galβ structures as targets. Gene transfer of alpha(1,3)galactosyltransferase into tumor cells has been been explored as a means of inducing α-galactosyl directed immune responses against tumors. The following references relate to this matter: Fang J., et al., “A Unique Chemoenzymatic Synthesis of α-Galactosyl Epitope Derivatives Containing Free Amino Groups: Efficient Separation and Further Manipulation,” J Org Chem, 64(11):4089-4094 (1999); and Janczuk A., et al., “Alpha-Gal Oligosaccharides: Chemistry and Potential Biomedical Application,” Curr Med Chem, 6(2):155-64 (1999); and Galili U., “Abnormal Expression of Alpha-Galactosyl Epitopes in Man. A Trigger for Autoimmune Processes?” Lancet, 2(8659):358-61 (1989); and Jager U., et al., “Induction of Complement Attack on Human Cells by Gal(Alphal,3)Gal Xenoantigen Expression as a Gene Therapy Approach to Cancer,” Gene Ther, 6(6):1073-83 (1999); and Artrip J. H., et al., “Target Cell Susceptibility to Lysis by Human Natural Killer Cells is Augmented by α(1,3)-Galactosyltransferase and Reduced by α(1,2)-Fucosyltransferase-,” J Biol Chem, 274(16):10717-10722 (1999); and Vaughan H. A., et al., “Gal alpha(1,3)Gal is the Major Xenoepitope Expressed on Pig Endothelial Cells Recognized by Naturally Occurring Cytotoxic Human Antibodies,” Transplantation, 58(8):879-82 (1994); and Inverardi L., et al., “Human Natural Killer Lymphocytes Directly Recognize Evolutionarily Conserved Oligosaccharide Ligands Expressed by Xenogeneic Tissues,” Transplantation, 63(9):1318-30 (1997); and Ni Y., et al., “Specificity and Prevalence of Natural Bovine Anti-Alpha Galactosyl (Gal^{α}1-6Glc or Gal^{α}-16Gal) Antibodies,” Clin Diagnostic Lab Immunol, 7(3):490-496 (2000); and LaTemple D. C. et al., “Synthesis of Alpha-Galactosyl Epitopes by Recombinant Alpha1,3galactosyl Transferase for Opsonization of Human Tumor Cell Vaccines by Anti-Galactose,” Cancer Res, 56(13):3069-74 (1996); and Galili U., et al., “Human Natural Anti-Alpha-Galactosyl IgG. II. The Specific Recognition of Alpha (1,3)-Linked Galactose Residues,” J Exp Med, 162:573-582 (1985); and Link C. J. Jr., et al., “Eliciting Hyperacute Xenograft Response to Treat Human Cancer: Alpha(1,3) Galactosyltransferase Gene Therapy,” Anti-cancer Res, 18(4A):2301-8 (1998); and the contents are hereby incorporated by reference in their entirety.

The intense innate immunity that pre-exists in humans to these epitopes can be targeted against tumors by selectively delivering masked terminal α-galactosyl-(1,3)Galβ structures to tumors. In a preferred embodiment, E is comprised of one or more masked terminal α-galactosyl-(1-3)Galβ structures. Addition of a bioreversible masking group to one or more of the hydroxy groups on the disaccharide will alter the conformation and preclude antibody binding. Unmasking following tumor localization will expose the epitope and trigger an intense antitumor response.

In a preferred embodiment (Ein14), E is comprised of the following structure:

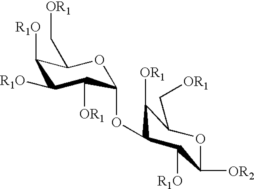

wherein R₁ is OH or a bioreversible masking group which when unmasked exposes the hydroxy group, and R₂ (which may bear additional sugar residues) is the site of linker attachment to the remainder of the drug. R₁ can be an ester, phosphate, acetal, carbonate, or any group which can generate the free hydroxy group by spontaneous or biochemical mechanisms.

In a preferred embodiment (Ein15), E has the following structure:

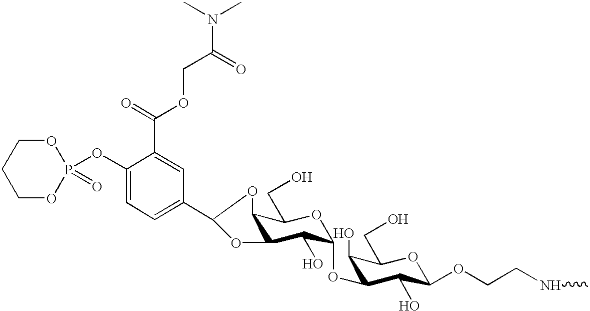

wherein the wavy line is the site of attachment to the remainder of the drug complex. Activation of the clock-like time delayed trigger by esterase will trigger acetal hydrolysis by stabilizing the carbocation formation at the benzylic carbon and unmask the antigen.

Multifactorial Targeting with Sets of Monofactorial Drugs and Multiple Set-MultiFactorial Targeting

Although a single property or characteristic is not unique to malignant cells the pattern of expression of multiple properties may provide almost absolute tumor specificity. Multifunctional drug delivery vehicles provide one means to accomplish multifactorial targeting. This section describes a complementary technology that may be used to achieve highly selective multifactorial targeting by using multiple independently targeted drugs to deliver multiple effector agents, wherein the effector agents individually have low toxicity, but jointly are highly toxic. This technology may be employed with monofactorially targeted drugs or with multifunctional drug delivery vehicles. When applied to multifunctional drug delivery vehicles, this technology will restrict the targeting domain of toxicity to cells that jointly express both sets of properties targeted by each multifunctional drug delivery vehicle.

This invention relates to the compositions, targets and methods of use of independent sets of targeted drugs; wherein the targeted drugs individually have low toxicity, but the combination of one or more of the drugs is potently toxic for cells that are jointly targeted. Any combination of effector agents that display potent synergystic toxicity, and which individually are of much lower toxicity may be employed. In this technology, multifatorial targeting occurs at the effector level.

Synergystic combinations of conventional drugs with enhanced toxicity are well known in cancer chemotherapy. Grosveld disclosed a means of targeting genes and regulatory elements, which functionally cooperate inside the cell using different independent targeting ligands. Combinations of targeted immunotoxins that exert synergystic toxicity are also known. The following references relate to this matter: U.S. Pat. No. 5,849,718 Dec. 15, 1998 Grosveld, “Targeting Complexes and Use Thereof”.; and Crews J. R., et al., “A Combination of Two Immunotoxins Exerts Synergistic Cytotoxic Activity Against Human Breast-Cancer Cell Lines,” Int J Cancer, 51:772-779 (1992) and the contents are hereby incorporated by reference in their entirety.

The combination of antimetabolites, which interfere with the denovo synthesis of a factor essential for cell growth and survival with inhibitors that block the salvage pathways related to the factor, may display striking synergystic toxicity. Examples include:

1.) Inhibitors of purine and pyrimidine synthesis in combination with nucleoside transport inhibitors; and

2.) Inhibitors of polyamine synthesis and polyamine transport inhibitors.

One embodiment of the present invention comprises a set of n targeted drugs referred to as “E1-T1” . . . “En-Tn” wherein E1 . . . En comprise effector groups which in combination exert synergystic toxicity, and T1 . . . Tn comprise different targeting ligands. (A large number of targeting ligands and tumor-selective targeting ligands have been detailed in other sections and apply to this embodiment of the present invention.) The present invention also relates to the method in which this set of drugs is administered, in combination, alone or in conjunction with non-targeted drugs that further potentiate selective toxicity for the treatment of neoplastic disease.

A preferred embodiment (referred to as embodiment “PET1”) of the present invention comprises the set of n different drugs referred to as “E1T1” . . . “EnTn” wherein EnTn is a compound comprised of one or more effector agents referred to as “En.v” having pharmacological activity designated as “PA” and wherein Tn comprises:

a) A group comprised of at least one structure referred to as a “targeting ligand” which selectively binds to a target receptor on the surface of the target cell or in the microenvironment of the target cell; and

And wherein the different drugs E1T1 . . . EnTn bind to different types of target receptors; and wherein the different effector groups E1 . . . En can evoke pharmacological activitites that are synergistic; wherein synergistic means that the pharmacological activity produced by the effector groups E1 . . . En is greater than the additive pharmacological activity of the individual effector groups acting independently;

and wherein n is at least two; and n=1,2,3,4,5,6,7,8,9,10,11,12,13,14 or about 15; preferably N is two or three;

and wherein v is 1, 2, 3, or about 4, and preferably v is 1 or 2;

and wherein the drugs E1T1 . . . EnTn are combined; wherein combined means that the drugs are present in the same solution (or liquid phase) or volume of space before being given to a patient or become so in a patient.

A preferred embodiment (embodiment PET2) of the above comprises the set of drugs E1-T1 . . . En-Tn wherein En-Tn is comprised of the following groups:

a) N1 targeting ligands, which can differ;

b) N2 masked intracellular transport ligands which can differ;

c) N3 triggers, which can differ, designated “detoxification triggers” wherein activation of the trigger decreases the pharmacological activity PA;

d) N4 effector agents which can differ;

e) N5 triggers which can differ, wherein activation of the trigger increases the pharmacological activity PA;

f) N6 intracellular trapping ligands or masked intracellular trapping ligands, which can differ;

and wherein:

N1=1, 2, 3, 4, 5, 6, 7, 8, 9, 10, or about 10;

N2=0, 1, 2, 3, 4, or 5;

N3=0, 1, 2, 3, 4, 5, or about 5;

N4=1, 2, 3, 4, 5, or about 5;

N5=0, 1, 2, 3, 4, 5, 6, 7, 8, 9, 10,orabout10;and

N6=0, 1, 2, 3, 4, 5, 6, 7, 8, 9, 10, or about 10;

and wherein the components are covalently coupled directly or by one or more linkers, and wherein the connectivity between groups can vary provided that the functionality of the different components remains intact and wherein the function of ligands is to bind to their respective receptors; the function of the triggers is to be activated and modulate drug activity, and the function of the effector agent is to evoke the pharmacological activity PA;

and wherein the linker lengths can be 1, 2, 3, 4, 5, 6, 7, 8, 9, 10, 11, 12, 13,14, 15, 16, 17, 18, 19, . . . 300 bond lengths or about 300 bond lengths; wherein the ( . . . ) are meant to represent the continuation of the sequence of numbers up to 300.

In a preferred embodiment (embodiment PET3) of the above:

N1=1, 2, 3, or 4;

N2=0, 1, or 2;

N3=0, 1, or 2;

N4=1, 2, or 3;

N5=0, 1, 2, or 3;

N6=1, 2, or 3;

Additional preferred embodiments (referred to as embodiment PET4.x wherein is X=the number of the line below for X=1, 2, 3 . . . 383) of EnTn are listed on each line below wherein:

1) N1=1, N2=0, N3=1, N4=1, N5=0, and N6=0

2) N1=1, N2=0, N3=0, N4=2, N5=0, and N6=0

3) N1=1, N2=0, N3=0, N4=3, N5=0, and N6=0

4) N1=1, N2=0, N3=0, N4=1, N5=1, and N6=0

5) N1=1, N2=0, N3=0, N4=1, N5=2, and N6=0

6) N1=1, N2=0, N3=0, N4=1, N5=3, and N6=0

7) N1=1, N2=0, N3=0, N4=1, N5=0, and N6=1

8) N1=1, N2=0, N3=1, N4=2, N5=0, and N6=0

9) N1=1, N2=0, N3=1, N4=3, N5=0, and N6=0

10) N1=1, N2=0, N3=1, N4=1, N5=1, and N6=0

11) N1=1, N2=0, N3=1, N4=1, N5=2, and N6=0

12) N1=1, N2=0, N3=1, N4=1, N5=3, and N6=0

13) N1=1, N2=0, N3=1, N4=1, N5=0, and N6=1

14) N1=1, N2=0, N3=1, N4=2, N5=1, and N6=0

15) N1=1, N2=0, N3=1, N4=2, N5=1, and N6=1

16) N1=1, N2=0, N3=1, N4=2, N5=2, and N6=0

17) N1=1, N2=0, N3=1, N4=2, N5=2, and N6=1

18) N1=1, N2=0, N3=1, N4=2, N5=3, and N6=0

19) N1=1, N2=0, N3=1, N4=2, N5=3, and N6=1

20) N1=1, N2=0, N3=1, N4=2, N5=0, and N6=1

21) N1=1, N2=0, N3=1, N4=3, N5=1, and N6=0

22) N1=1, N2=0, N3=1, N4=3, N5=1, and N6=1

23) N1=1, N2=0, N3=1, N4=3, N5=2, and N6=0

24) N1=1, N2=0, N3=1, N4=3, N5=2, and N6=1

25) N1=1, N2=0, N3=1, N4=3, N5=3, and N6=0

26) N1=1, N2=0, N3=1, N4=3, N5=3, and N6=1

27) N1=1, N2=0, N3=1, N4=3, N5=0, and N6=1

28) N1=1, N2=0, N3=1, N4=1, N5=1, and N6=1

29) N1=1, N2=0, N3=1, N4=1, N5=2, and N6=1

30) N1=1, N2=0, N3=1, N4=1, N5=3, and N6=1

31) N1=1, N2=1, N3=0, N4=1, N5=0, and N6=0

32) N1=1, N2=1, N3=0, N4=2, N5=0, and N6=0

33) N1=1, N2=1, N3=0, N4=3, N5=0, and N6=0

34) N1=1, N2=1, N3=0, N4=1, N5=1, and N6=0

35) N1=1, N2=1, N3=0, N4=1, N5=2, and N6=0

36) N1=1, N2=1, N3=0, N4=1, N5=3, and N6=0

37) N1=1, N2=1, N3=0, N4=1, N5=0, and N6=1

38) N1=1, N2=1, N3=0, N4=2, N5=1, and N6=0

39) N1=1, N2=1, N3=0, N4=2, N5=1, and N6=1

40) N1=1, N2=1, N3=0, N4=2, N5=2, and N6=0

41) N1=1, N2=1, N3=0, N4=2, N5=2, and N6=1

42) N1=1, N2=1, N3=0, N4=2, N5=3, and N6=0

43) N1=1, N2=1, N3=0, N4=2, N5=3, and N6=1

44) N1=1, N2=1, N3=0, N4=2, N5=0, and N6=1

45) N1=1, N2=1, N3=0, N4=3, N5=1, and N6=0

46) N1=1, N2=1, N3=0, N4=3, N5=1, and N6=1

47) N1=1, N2=1, N3=0, N4=3, N5=2, and N6=0

48) N1=1, N2=1, N3=0, N4=3, N5=2, and N6=1

49) N1=1, N2=1, N3=0, N4=3, N5=3, and N6=0

50) N1=1, N2=1, N3=0, N4=3, N5=3, and N6=1

51) N1=1, N2=1, N3=0, N4=3, N5=0, and N6=1

52) N1=1, N2=1, N3=0, N4=1, N5=1, and N6=1

53) N1=1, N2=1, N3=0, N4=1, N5=2, and N6=1

54) N1=1, N2=1, N3=0, N4=1, N5=3, and N6=1

55) N1=1, N2=1, N3=1, N4=1, N5=0, and N6=0

56) N1=1, N2=1, N3=1, N4=2, N5=0, and N6=0

57) N1=1, N2=1, N3=1, N4=3, N5=0, and N6=0

58) N1=1, N2=1, N3=1, N4=1, N5=1, and N6=0

59) N1=1, N2=1, N3=1, N4=1, N5=2, and N6=0

60) N1=1, N2=1, N3=1, N4=1, N5=3, and N6=0

61) N1=1, N2=1, N3=1, N4=1, N5=0, and N6=1

62) N1=1, N2=1, N3=1, N4=2, N5=1, and N6=0

63) N1=1, N2=1, N3=1, N4=2, N5=1, and N6=1

64) N1=1, N2=1, N3=1, N4=2, N5=2, and N6=0

65) N1=1, N2=1, N3=1, N4=2, N5=2, and N6=1

66) N1=1, N2=1, N3=1, N4=2, N5=3, and N6=0

67) N1=1, N2=1, N3=1, N4=2, N5=3, and N6=1

68) N1=1, N2=1, N3=1, N4=2, N5=0, and N6=1

69) N1=1, N2=1, N3=1, N4=3, N5=1, and N6=0

70) N1=1, N2=1, N3=1, N4=3, N5=1, and N6=1

71) N1=1, N2=1, N3=1, N4=3, N5=2, and N6=0

72) N1=1, N2=1, N3=1, N4=3, N5=2, and N6=1

73) N1=1, N2=1, N3=1, N4=3, N5=3, and N6=0

74) N1=1, N2=1, N3=1, N4=3, N5=3, and N6=1

75) N1=1, N2=1, N3=1, N4=3, N5=0, and N6=1

76) N1=1, N2=1, N3=1, N4=1, N5=1, and N6=1

77) N1=1, N2=1, N3=1, N4=1, N5=2, and N6=1

78) N1=1, N2=1, N3=1, N4=1, N5=3, and N6=1

79) N1=1, N2=0, N3=0, N4=2, N5=1, and N6=0

80) N1=1, N2=0, N3=0, N4=2, N5=2, and N6=0

81) N1=1, N2=0, N3=0, N4=2, N5=3, and N6=0

82) N1=1, N2=0, N3=0, N4=2, N5=0, and N6=1

83) N1=1, N2=0, N3=0, N4=3, N5=1, and N6=0

84) N1=1, N2=0, N3=0, N4=3, N5=2, and N6=0

85) N1=1, N2=0, N3=0, N4=3, N5=3, and N6=0

86) N1=1, N2=0, N3=0, N4=3, N5=0, and N6=1

87) N1=1, N2=0, N3=0, N4=1, N5=2, and N6=1

88) N1=1, N2=0, N3=0, N4=1, N5=3, and N6=1

89) N1=1, N2=0, N3=0, N4=2, N5=1, and N6=1

90) N1=1, N2=0, N3=0, N4=2, N5=2, and N6=1

91) N1=1, N2=0, N3=O, N4=2, N5=3, and N6=1

92) N1=1, N2=0, N3=0, N4=3, N5=1, and N6=1

93) N1=1, N2=0, N3=0, N4=3, N5=2, and N6=1

94) N1=1, N2=0, N3=0, N4=3, N5=3, and N6=1

95) N1=1, N2=0, N3=0, N4=1, N5=1, and N6=1

96) N1=2, N2=0, N3=0, N4=1, N5=0, and N6=0

97) N1=2, N2=0, N3=1, N4=1, N5=0, and N6=0

98) N1=2, N2=0, N3=0, N4=2, N5=0, and N6=0

99) N1=2, N2=0, N3=0, N4=3, N5=0, and N6=0

100) N1=2, N2=0, N3=0, N4=1, N5=1, and N6=0

101) N1 =2, N2=0, N3=0, N4=1, N5=2, and N6=0

102) N1=2, N2=0, N3=0, N4=1, N5=3, and N6=0

103) N1=2, N2=0, N3=0, N4=1, N5=0, and N6=1

104) N1=2, N2=0, N3=1, N4=2, N5=0, and N6=0

105) N1=2, N2=0, N3=1, N4=3, N5=0, and N6=0

106) N1=2, N2=0, N3=1

, N4=1, N5=1, and N6=0

107) N1=2, N2=0, N3=1, N4=1, N5=2, and N6=0

108) N1=2, N2=0, N3=1, N4=1, N5=3, and N6=0

109) N1=2, N2=0, N3=0, N4=1, N5=0, and N6=1

110) N1=2, N2=0, N3=1, N4=2, N5=1, and N6=0

111) N1=2, N2=0, N3=1, N4=2, N5=1, and N6=1

112) N1=2, N2=0, N3=1, N4=2, N5=2, and N6=0

113) N1=2, N2=0, N3=1, N4=2, N5=2, and N6=1

114) N1=2, N2=0, N3=1, N4=2, N5=3, and N6=0

115) N1=2, N2=0, N3=1, N4=2, N5=3, and N6=1

116) N1=2, N2=0, N3=1, N4=2, N5=0, and N6=1

117) N1=2, N2=0, N3=1, N4=3, N5=1, and N6=0

118) N1=2, N2=0, N3=1, N4=3, N5=1, and N6=1

119) N1=2, N2=0, N3=1, N4=3, N5=2, and N6=0

120) N1=2, N2=0, N3=1, N4=3, N5=2, and N6=1

121) N1=2, N2=0, N3=1, N4=3, N5=3, and N6=0

122) N1=2, N2=0, N3=1, N4=3, N5=3, and N6=1

123) N1=2, N2=0, N3=1, N4=3, N5=0, and N6=1

124) N1=2, N2=0, N3=1, N4=1, N5=1, and N6=1

125) N1=2, N2=0, N3=1, N4=1, N5=2, and N6=1

126) N1=2, N2=0, N3=1, N4=1, N5=3, and N6=1

127) N1=2, N2=1, N3=0, N4=1, N5=0, and N6=0

128) N1=2, N2=1, N3=0, N4=2, N5=0, and N6=0

129) N1=2, N2=1, N3=0, N4=3, N5=0, and N6=0

130) N1=2, N2=1, N3=0, N4=1, N5=1, and N6=0

131) N1=2, N2=1, N3=0, N4=1, N5=2, and N6=0

132) N1=2, N2=1, N3=0, N4=1, N5=3, and N6=0

133) N1=2, N2=1, N3=0, N4=1, N5=0, and N6=1

134) N1=2, N2=1, N3=0, N4=2, N5=1, and N6=0

135) N1=2, N2=1, N3=0, N4=2, N5=1, and N6=1

136) N1=2, N2=1, N3=0, N4=2, N5=2, and N6=0

137) N1=2, N2=1, N3=0, N4=2, N5=2, and N6=1

138) N1=2, N2=1, N3=0, N4=2, N5=3, and N6=0

139) N1=2, N2=1, N3=0, N4=2, N5=3, and N6=1

140) N1=2, N2=1, N3=0, N4=2, N5=0, and N6=1

141) N1=2, N2=1, N3=0, N4=3, N5=1, and N6=0

142) N1=2, N2=1, N3=0, N4=3, N5=1, and N6=1

143) N1=2, N2=1, N3=0, N4=3, N5=2, and N6=0

144) N1=2, N2=1, N3=0, N4=3, N5=2, and N6=1

145) N1=2, N2=1, N3=0, N4=3, N5=3, and N6=0

146) N1=2, N2=1, N3=0, N4=3, N5=3, and N6=1

147) N1=2, N2=1, N3=0, N4=3, N5=0, and N6=1

148) N1=2, N2=1, N3=0, N4=1, N5=1, and N6=1

149) N1=2, N2=1, N3=0, N4=1, N5=2, and N6=1

150) N1=2, N2=1, N3=0, N4=1, N5=3, and N6=1

151) N1=2, N2=1, N3=1, N4=1, N5=0, and N6=0

152) N1=2, N2=1, N3=1, N4=2, N5=0, and N6=0

153) N1=2, N2=1, N3=1, N4=3, N5=0, and N6=0

154) N1=2, N2=1, N3=1, N4=1, N5=1, and N6=0

155) N1=2, N2=1, N3=1, N4=1, N5=2, and N6=0

156) N1=2, N2=1, N3=1, N4=1, N5=3, and N6=0

157) N1=2, N2=1, N3=1, N4=1, N5=0, and N6=1

158) N1=2, N2=1, N3=1, N4=2, N5=1, and N6=0

159) N1=2, N2=1, N3=1, N4=2, N5=1, and N6=1

160) N1=2, N2=1, N3=1, N4=2, N5=2, and N6=0

161) N1=2, N2=1, N3=1, N4=2, N5=2, and N6=1

162) N1=2, N2=1, N3=1, N4=2, N5=3, and N6=0

163) N1=2, N2=1, N3=1, N4=2, N5=3, and N6=1

164) N1=2, N2=1, N3=1, N4=2, N5=0, and N6=1

165) N1=2, N2=1, N3=1, N4=3, N5=1, and N6=0

166) N1=2, N2=1, N3=1, N4=3, N5=1, and N6=1

167) N1=2, N2=1, N3=1, N4=3, N5=2, and N6=0

168) N1=2, N2=1, N3=1, N4=3, N5=2, and N6=1

169) N1=2, N2=1, N3=1, N4=3, N5=3, and N6=0

170) N1=2, N2=1, N3=1, N4=3, N5=3, and N6=1

171) N1=2, N2=1, N3=1, N4=3, N5=0, and N6=1

172) N1=2, N2=1, N3=1, N4=1, N5=1, and N6=1

173) N1=2, N2=1, N3=1, N4=1, N5=2, and N6=1

174) N1=2, N2=1, N3=1, N4=1, N5=3, and N6=1

175) N1=2, N2=0, N3=0, N4=2, N5=1, and N6=0

176) N1=2, N2=0, N3=0, N4=2, N5=2, and N6=0

177) N1=2, N2=0, N3=0, N4=2, N5=3, and N6=0

178) N1=2, N2=0, N3=0, N4=2, N5=0, and N6=1

179) N1=2, N2=0, N3=0, N4=3, N5=1, and N6=0

180) N1=2, N2=0, N3=0, N4=3, N5=2, and N6=0

181) N1=2, N2=0, N3=0, N4=3, N5=3, and N6=0

182) N1=2, N2=0, N3=0, N4=3, N5=0, and N6=1

183) N1=2, N2=0, N3=0, N4=1, N5=2, and N6=1

184) N1=2, N2=0, N3=0, N4=1, N5=3, and N6=1

185) N1=2, N2=0, N3=0, N4=2, N5=1, and N6=0

186) N1=2, N2=0, N3=0, N4=2, N5=2, and N6=1

187) N1=2, N2=0, N3=0, N4=2, N5=3, and N6=1

188) N1=2, N2=0, N3=0, N4=3, N5=1, and N6=1

189) N1=2, N2=0, N3=0, N4=3, N5=2, and N6=1

190) N1=2, N2=0, N3=0, N4=3, N5=3, and N6=1

191) N1=2, N2=0, N3=0, N4=1, N5=1, and N6=1

192) N1=3, N2=0, N3=0, N4=1, N5=0, and N6=0

193) N1=3, N2=0, N3=1, N4=1, N5=0, and N6=0

194) N1=3, N2=0, N3=0, N4=2, N5=0, and N6=0

195) N1=3, N2=0, N3=0, N4=3, N5=0, and N6=0

196) N1=3, N2=0, N3=0, N4=1, N5=1, and N6=0

197) N1=3, N2=0, N3=0, N4=1, N5=2, and N6=0

198) N1=3, N2=0, N3=0, N4=1, N5=3, and N6=0

199) N1=3, N2=0, N3=0, N4=1, N5=0, and N6=1

200) N1=3, N2=0, N3=1, N4=2, N5=0, and N6=0

201) N1=3, N2=0, N3=1, N4=3, N5=0, and N6=0

202) N1=3, N2=0, N3=1, N4=1, N5=1, and N6=0

203) N1=3, N2=0, N3=1, N4=1, N5=2, and N6=0

204) N1=3, N2=0, N3=1, N4=1, N5=3, and N6=0

205) N1=3, N2=0, N3=1, N4=1, N5=0, and N6=1

206) N1=3, N2=0, N3=1, N4=2, N5=1, and N6=0

207) N1=3, N2=0, N3=1, N4=2, N5=1, and N6=1

208) N1=3, N2=0, N3=1, N4=2, N5=2, and N6=0

209) N1=3, N2=0, N3=1, N4=2, N5=2, and N6=1

210) N1 =3, N2=0, N3=1, N4=2, N5=3, and N6=0

211) N1=3, N2=0, N3=1, N4=2, N5=3, and N6=1

212) N1=3, N2=0, N3=1, N4=2, N5=0, and N6=1

213) N1=3, N2=0, N3=1, N4=3, N5=1, and N6=0

214) N1=3, N2=0, N3=1, N4=3, N5=1, and N6=1

215) N1=3, N2=0, N3=1, N4=3, N5=2, and N6=0

216) N1=3, N2=0, N3=1, N4=3, N5=2, and N6=1

217) N1=3, N2=0, N3=1, N4=3, N5=3, and N6=0

218) N1=3, N2=0, N3=1, N4=3, N5=3, and N6=1

219) N1=3, N2=0, N3=1, N4=3, N5=0, and N6=1

220) N1=3, N2=0, N3=1, N4=1, N5=1, and N6=1

221) N1=3, N2=0, N3=1, N4=1, N5=2, and N6=1

222) N1=3, N2=0, N3=1, N4=1, N5=3, and N6=1

223) N1=3, N2=1, N3=0, N4=1, N5=0, and N6=0

224) N1=3, N2=1, N3=0, N4=2, N5=0, and N6=0

225) N1=3, N2=1, N3=0, N4=3, N5=0, and N6=0

226) N1=3, N2=1, N3=0, N4=1, N5=1, and N6=0

227) N1=3, N2=1, N3=0, N4=1, N5=2, and N6=0

228) N1=3, N2=1, N3=0, N4=1, N5=3, and N6=0

229) N1=3, N2=1, N3=0, N4=1, N5=0, and N6=1

230) N1=3, N2=1, N3=0, N4=2, N5=1, and N6=0

231) N1=3, N2=1, N3=0, N4=2, N5=1, and N6=1

232) N1=3, N2=1, N3=0, N4=2, N5=2, and N6=0

233) N1=3, N2=1, N3=0, N4=2, N5=2, and N6=1

234) N1=3, N2=1, N3=0, N4=2, N5=3, and N6=0

235) N1=3, N2=1, N3=0, N4=2, N5=3, and N6=1

236) N1=3, N2=1, N3=0, N4=2, N5=0, and N6=1

237) N1=3, N2=1, N3=0, N4=3, N5=1, and N6=0

238) N1=3, N2=1, N3=0, N4=3, N5=1, and N6=1

239) N1=3, N2=1, N3=0, N4=3, N5=2, and N6=0

240) N1=3, N2=1, N3=0, N4=3, N5=2, and N6=1

241) N1=3, N2=1, N3=0, N4=3, N5=3, and N6=0

242) N1=3, N2=1, N3=0, N4=3, N5=3, and N6=1

243) N1=3, N2=1, N3=0, N4=3, N5=0, and N6=1

244) N1=3, N2=1, N3=0, N4=1, N5=1, and N6=1

245) N1=3, N2=1, N3=0, N4=1, N5=2, and N6=1

246) N1=3, N2=1, N3=0, N4=1, N5=3, and N6=1

247) N1=3, N2=1, N3=1, N4=1, N5=0, and N6=0

248) N1=3, N2=1, N3=1, N4=2, N5=0, and N6=0

249) N1=3, N2=1, N3=1, N4=3, N5=0, and N6=0

250) N1=3, N2=1, N3=1, N4=1, N5=1, and N6=0

251) N1=3, N2=1, N3=1, N4=1, N5=2, and N6=0

252) N1=3, N2=1, N3=1, N4=1, N5=3, and N6=0

253) N1=3, N2=1, N3=1, N4=1, N5=0, and N6=1

254) N1=3, N2=1, N3=1, N4=2, N5=1, and N6=0

255) N1=3, N2=1, N3=1, N4=2, N5=1, and N6=1

256) N1=3, N2=1, N3=1, N4=2, N5=2, and N6=0

257) N1=3, N2=1, N3=1, N4=2, N5=2, and N6=1

258) N1=3, N2=1, N3=1, N4=2, N5=3, and N6=0

259) N1=3, N2=1, N3=1, N4=2, N5=3, and N6=1

260) N1=3, N2=1, N3=1, N4=2, N5=0, and N6=1

261) N1=3, N2=1, N3=1, N4=3, N5=1, and N6=0

262) N1=3, N2=1, N3=1, N4=3, N5=1, and N6=1

263) N1=3, N2=1, N3=1, N4=3, N5=2, and N6=0

264) N1=3, N2=1, N3=1, N4=3, N5=2, and N6=1

265) N1=3, N2=1, N3=1, N4=3, N5=3, and N6=0

266) N1=3, N2=1, N3=1, N4=3, N5=3, and N6=1

267) N1=3, N2=1, N3=1, N4=3, N5=0, and N6=1

268) N1=3, N2=1, N3=1, N4=1, N5=1, and N6=1

269) N1=3, N2=1, N3=1, N4=1, N5=2, and N6=1

270) N1=3, N2=1, N3=1, N4=1, N5=3, and N6=1

271) N1=3, N2=0, N3=0, N4=2, N5=1, and N6=0

272) N1=3, N2=0, N3=0, N4=2, N5=2, and N6=0

273) N1=3, N2=0, N3=0, N4=2, N5=3, and N6=0

274) N1=3, N2=0, N3=0, N4=2, N5=0, and N6=1

275) N1=3, N2=0, N3=0, N4=3, N5=1, and N6=0

276) N1=3, N2=0, N3=0, N4=3, N5=2, and N6=0

277) N1=3, N2=0, N3=0, N4=3, N5=3, and N6=0

15 278) N1=3, N2=0, N3=0, N4=3, N5=0, and N6=0

279) N1=3, N2=0, N3=0, N4=1, N5=2, and N6=1

280) N1=3, N2=0, N3=0, N4=1, N5=3, and N6=1

281) N1=3, N2=0, N3=0, N4=2, N5=3, and N6=1

282) N1=3, N2=0, N3=0, N4=2, N5=2, and N6=1

283) N1=3, N2=0, N3=0, N4=2, N5=3, and N6=1

284) N1=3, N2=0, N3=0, N4=3, N5=3, and N6=1

285) N1=3, N2=0, N3=0, N4=3, N5=2, and N6=1

286) N1=3, N2=0, N3=0, N4=3, N5=3, and N6=1

287) N1=3, N2=0, N3=0, N4=1, N5=1, and N6=1

288) N1=4, N2=0, N3=0, N4=1, N5=0, and N6=0

289) N1=4, N2=0, N3=1, N4=1, N5=0, and N6=0

290) N1=4, N2=0, N3=0, N4=2, N5=0, and N6=0

291) N1=4, N2=0, N3=0, N4=3, N5=0, and N6=0

292) N1=4, N2=O, N3=0, N4=1, N5=1, and N6=0

293) N1=4, N2=0, N3=0, N4=1, N5=2, and N6=0

294) N1 =4, N2=0, N3=0, N4=1, N5=3, and N6=0

295) N1=4, N2=0, N3=0, N4=1, N5=0, and N6=1

296) N1=4, N2=0, N3=1, N4=2, N5=0, and N6=0

297) N1=4, N2=0, N3=1, N4=3, N5=0, and N6=0

298) N1=4, N2=0, N3=1, N4=1, N5=1, and N6=0

299) N1=4, N2=0, N3=1, N4=1, N5=2, and N6=0

300) N1=4, N2=0, N3=1, N4=1, N5=3, and N6=0

301) N1=4, N2=0, N3=1, N4=1, N5=0, and N6=1

302) N1=4, N2=0, N3=1, N4=2, N5=1, and N6=0

303) N1=4, N2=0, N3=1, N4=2, N5=1, and N6=1

304) N1=4, N2=0, N3=1, N4=2, N5=2, and N6=0

305) N1=4, N2=0, N3=1, N4=2, N5=2, and N6=1

306) N1=4, N2=0, N3=1, N4=2, N5=3, and N6=0

307) N1=4, N2=0, N3=1, N4=2, N5=3, and N6=1

308) N1=4, N2=0, N3=1, N4=2, N5=0, and N6=1

309) N1=4, N2=0, N3=1, N4=3, N5=1, and N6=0

310) N1=4, N2=0, N3=1, N4=3, N5=1, and N6=1

311) N1=4, N2=0, N3=1, N4=3, N5=2, and N6=0

312) N1=4, N2=0, N3=1, N4=3, N5=2, and N6=1

313) N1=4, N2=0, N3=1, N4=3, N5=3, and N6=0

314) N1=4, N2=0, N3=1, N4=3, N5=3, and N6=1

315) N1=4, N2=0, N3=1, N4=3, N5=0, and N6=1

316) N1=4, N2=0, N3=1, N4=1, N5=1, and N6=1

317) N1=4, N2=0, N3=1, N4=1, N5=2, and N6=1

318) N1=4, N2=0, N3=1, N4=1, N5=3, and N6=1

319) N1=4, N2=1, N3=0, N4=1, N5=0, and N6=0

320) N1=4, N2=1, N3=0, N4=2, N5=0, and N6=0

321) N1=4, N2=1, N3=0, N4=3, N5=0, and N6=0

322) N1=4, N2=1, N3=0, N4=1, N5=1, and N6=0

323) N1=4, N2=1, N3=0, N4=1, N5=2, and N6=0

324) N1=4, N2=1, N3=0, N4=1, N5=3, and N6=0

325) N1=4, N2=1, N3=0, N4=1, N5=0, and N6=1

326) N1=4, N2=1, N3=0, N4=2, N5=1, and N6=0

327) N1=4, N2=1, N3=0, N4=2, N5=1, and N6=1

328) N1=4, N2=1, N3=0, N4=2, N5=2, and N6=0

329) N1=4, N2=1, N3=0, N4=2, N5=2, and N6=1

330) N1=4, N2=1, N3=0, N4=2, N5=3, and N6=0

331) N1=4, N2=1, N3=0, N4=2, N5=3, and N6=1

332) N1=4, N2=1, N3=0, N4=2, N5=0, and N6=1

333) N1=4, N2=1, N3=0, N4=3, N5=1, and N6=0

334) N1=4, N2=1, N3=0, N4=3, N5=1, and N6=1

335) N1=4, N2=1, N3=0, N4=3, N5=2, and N6=0

336) N1=4, N2=1, N3=0, N4=3, N5=2, and N6=1

337) N1=4, N2=1, N3=0, N4=3, N5=3, and N6=0

338) N1=4, N2=1, N3=0, N4=3, N5=3, and N6=1

339) N1=4, N2=1, N3=0, N4=3, N5=0, and N6=1

340) N1=4, N2=1, N3=0, N4=1, N5=1, and N6=1

341) N1=4, N2=1, N3=0, N4=1, N5=2, and N6=1

342) N1=4, N2=1, N3=0, N4=1, N5=3, and N6=1

343) N1=4, N2=1, N3=1, N4=1, N5=0, and N6=0

344) N1=4, N2=1, N3=1, N4=2, N5=0, and N6=0

345) N1=4, N2=1, N3=1, N4=3, N5=0, and N6=0

346) N1=4, N2=1, N3=1, N4=1, N5=1, and N6=0

347) N1=4, N2=1, N3=1, N4=1, N5=2, and N6=0

348) N1=4, N2=1, N3=1, N4=1, N5=3, and N6=0

349) N1=4, N2=1, N3=1, N4=1, N5=0, and N6=1

350) N1=4, N2=1, N3=1, N4=2, N5=1, and N6=0

351) N1=4, N2=1, N3=1, N4=2, N5=1, and N6=1

352) N1=4, N2=1, N3=1, N4=2, N5=2, and N6=0

353) N1=4, N2=1, N3=1, N4=2, N5=2, and N6=1

354) N1=4, N2=1, N3=1, N4=2, N5=3, and N6=0

355) N1=4, N2=1, N3=1, N4=2, N5=3, and N6=1

356) N1=4, N2=1, N3=1, N4=2, N5=0, and N6=1

357) N1=4, N2=1, N3=1, N4=3, N5=1, and N6=0

358) N1=4, N2=1, N3=1, N4=3, N5=0, and N6=1

359) N1=4, N2=1, N3=1, N4=3, N5=2, and N6=0

360) N1=4, N2=1, N3=1, N4=3, N5=2, and N6=1

361) N1=4, N2=1, N3=1, N4=3, N5=3, and N6=0

362) N1=4, N2=1, N3=1, N4=3, N5=3, and N6=1

363) N1=4, N2=1, N3=1, N4=3, N5=0, and N6=1

364) N1=4, N2=1, N3=1, N4=1, N5=1, and N6=1

365) N1=4, N2=1, N3=1, N4=1, N5=2, and N6=1

366) N1=4, N2=1, N3=1, N4=1, N5=3, and N6=1

367) N1=4, N2=0, N3=0, N4=2, N5=1, and N6=0

368) N1=4, N2=0, N3=0, N4=2, N5=2, and N6=0

369) N1=4, N2=0, N3=0, N4=2, N5=3, and N6=0

370) N1=4, N2=0, N3=0, N4=2, N5=0, and N6=1

371) N1=4, N2=0, N3=0, N4=3, N5=1, and N6=0

372) N1=4, N2=0, N3=0, N4=3, N5=2, and N6=0

373) N1=4, N2=0, N3=0, N4=3, N5=3, and N6=0

374) N1=4, N2=0, N3=0, N4=3, N5=0, and N6=1

375) N1=4, N2=0, N3=0, N4=1, N5=2, and N6=1

376) N1=4, N2=0, N3=0, N4=1, N5=3, and N6=1

377) N1=4, N2=0, N3=0, N4=2, N5=1, and N6=1

378) N1=4, N2=0, N3=0, N4=2, N5=2, and N6=1

379) N1=4, N2=0, N3=0, N4=2, N5=3, and N6=1

380) N1=4, N2=0, N3=0, N4=3, N5=1, and N6=1

381) N1=4, N2=0, N3=0, N4=3, N5=2, and N6=1

82) N1=4, N2=0, N3=0, N4=3, N5=3, and N6=1

383) N1=4, N2=0, N3=0, N4=1, N5=1, and N6=1

The different structures that can comprise the components are described in other sections of this document that detail components for ET.

In a preferred embodiment N1=1 and N4=1.

In a preferred embodiment, The targeting ligands are selective for receptors increased on tumor cells and the efector agents are drugs that exert synergistic toxicity. In a preferred embodiment the effector groups E1 . . . En exert synergystic toxicity by inhibiting the denovo synthesis of vital cellular factors and also inhibit salvage pathways related to these factors. In a preferred embodiment, at least one component of the set of effector groups En functions outside the cells and inhibits salvage pathways.

In a preferred embodiment, E1 . . . En inhibit the denovo synthesis of purine and or pyrimidine metabolites and related uptake and salvage pathways. In preferred embodiments, E1 . . . En inhibit denovo synthesis by inhibiting one or more of the following enzymes:

1.) thymidylate synthase

2.) ribonucleotide reductase

3.) glycinamide ribonucleotide transformylase

4.) 5-aminoimidazole-4-carboxamide ribonucleotide transferase

5.) dihydroorotate dehydrogenase

6.) carbamoyl phosphate synthetase

7.) orotidine-5′-phosphate decarboxylase

8.) inosine 5′-monophosphate dehydrogenase

9.) aspartate transcarbamylase

and inhibit salvage pathways by inhibiting one or more of the following:

1.) nucleoside transporter proteins

2.) thymidine kinase

3.) uridine/cytidine kinase

4.) deoxycytidine kinase

5.) deoxyguanosine kinase

6.) hypoxanthine-guanine phosphoribosyltransferase

7.) xanthine-guanine phosphoribosyltransferase

8.) adenine phosphoribosyltransferase

In a preferred embodiment, the targeted set of denovo and salvage pathway inhibitors are used in conjunction with a non-targeted analog related to the inhibited pathways that can be taken up by cells even in the presence of the salvage pathway inhibitors; and wherein the non-targeted inhibitor provides additional synergystic toxicity.

In a preferred embodiment, the set E1 . . . En can inhibit both thymidine monophosphate synthesis and thymidine transport, and azidothymidine (AZT) is administered concurrently in a non-targeted manner.

AZT, which enters cells by a mechanism independent of the nucleoside transporter system is known to inhibit thymidine kinase and potentiate the toxicity of inhibitors of denovo thymidine synthesis. The following references relate to this subject matter: Weber G., et al., “Regulation of De Novo and Salvage Pathways in Chemotherapy,” Adv Enzyme Regul, 31:45-67 (1991); Weber G., et al., “Salvage Capacity of Hepatoma 3924A and Action of Dipyridamole,” Adv Enzyme Regul, 21:53-69 (1983); Zimmerman T. P., et al., “3′-azido-3′-deoxythymidine. An Unusual Nucleoside Analogue that Permeates the Membrane of Human Erythrocytes and Lymphocytes by Nonfacilitated Diffusion,” J Biol Chem, 262(12):5748-54 (1987); Chan T. C. K., et al., “Permeation and Metabolism of Anti-HIV and Endogenous Nucleosides in Human Immune Effector Cells,” Biochemical Pharmacology, 46(2):273-278 (1993); Betageri G. V., et al., “Effect of Dipyridamole on Transport and Phosphorylation of Thymidine and 3′-azido-3′-deoxythymidine in Human Monocyte/Macrophages,” Biochemical Pharmacology, 404:867-870 (1990); Van Mouwerik T. J., et al., “Augmentation of Methotrexate Cytotoxicity in Human Colon Cancer Cells Achieved Through Inhibition of Thymidine Salvage by Dipyridamole,” Biochemical Pharmacology, 36(6):809-814 (1987); Andreuccetti M., et al., “Azidothymidine in Combination with 5-fluorouracil in Human Colorectal Cell Lines: In Vitro Synergistic Cytotoxicity and DNS-Induced Strand-Breaks,” Eur J Cancer, 32A(7):1219-26 (1996); Zhen Y. S., et al., “Azidothymidine and Dipyridamole as Biochemical Response Modifiers: Synergism with Methotrexate and 5-Fluorouracil in Human Colon and Pancreatic Carcinoma Cells,” Oncol Res, 4(2):73-8 (1992); Lehman N. L.; Danenberg P. V., “Modulation of RTX Cytotoxicity by Thymidine and Dipyridamole in Vitro: Implications for Chemotherapy,” Cancer Chemother Pharmacol, 45(2):142-8 (2000); Smith P. G., et al., “Dipyridamole Potentiates the in Vitro Activity of MTA (LY231514) by Inhibition of Thymidine Transport,” Br J Cancer, 82(4):924-30 (2000); Weber G., et al., “AZT: A Biochemical Response Modifier of Methotrexate and 5-Fluorouracil Cytotoxicity in Human Ovarian and Pancreatic Carcinoma Cells,” Cancer Commun, 3(4):127-32 (1991); Weber G., et al., “Azidothymidine Inhibition of Thymidine Kinase and Synergistic Cytotoxicity with Methotrexate and 5-Fluorouracil in Rat Hepatoma and Human Colon Cancer Cells,” Cancer Commun, 2(4):129-33 (1990); Zimmerman T. P., et al., “Inhibition of Thymidine Transport by 3′-azido-3′-deoxythymidine and its Metabolites,” Oncol Res, 5(12):483-7 (1993), the contents of which are incorporated herein by reference in their entirety.

A preferred embodiment of the present invention consists of the set of targeted drugs E1-T1 and E2-T2, wherein E1 comprises an inhibitor to thymidylate synthase and E2 comprises an inhibitor to nucleoside transporters. A preferred embodiment of E1 is based on the compound 1843U89 which is an extremely potent inhibitor of thymidylate synthase with a Ki of 90 pM. The following references relate to this subject matter: Duch D. S., et al., “Biochemical and Cellular Pharmacology of 1843U89, a Novel Benzoquinazoline Inhibitor of Thymidylate Synthase,” Cancer Res, 53(4):810-8 (1993); Stout T. J.; Stroud R. M., “The complex of the Anti-Cancer Therapeutic, BW1843U89, with Thymidylate Synthase at 2.0 a Resolution: Implications for a New Mode of Inhibition,” Structure, 4(1):67-77 (1996), the contents of which are incorporated herein by reference in their entirety.

In a preferred embodiment E1 comprises the following structure referred to as 1E1.1:

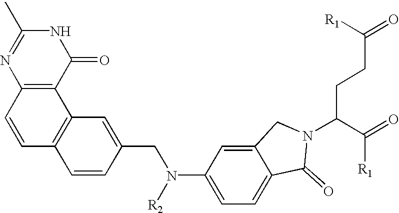

wherein R₁ is OH or the site of attachment of a linker or trigger connected to the remainder of the drug complex, and R₂ is H or the site of attachment of a trigger; and wherein the trigger, when activated, cleaves the R₁—C bond or the R₂—N bond.

And, E2 comprises the structure below referred to as embodiment 1 E2.1:

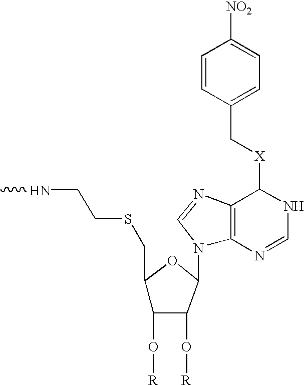

wherein R is H or a bioreversible hydroxy masking group that undergoes spontaneous or enzymatically triggered cleavage to expose the free hydroxy moiety; and wherein the wavy line is the site of linker attachment to the remainder of the drug; and wherein X is NH or S. The masking group can allow the targeting group T2 of the drug rather than E2 to define the targeting specificity. The principle is exactly the same as described previously for the case of masked intracellular transporter ligands.

Or E2 comprises the structure below referred to as embodiment 1 E2.2:

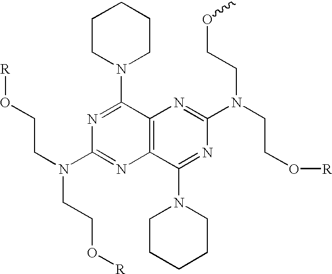

wherein the wavy line is the site of linker attachment to the remainder of the drug complex, and R is H or a bioreversible hydroxy masking group or masking trigger that undergoes spontaneous or enzymatically triggered cleavage to expose the free hydroxy moiety.

This structure is based upon the ability of dipyridamole to block nucleoside transporter function. The masking group can be employed to allow the targeting group T2 of the drug rather than E2 to define the targeting specificity. In addition, the masking group can prevent the tight binding of the dipyridamole moiety to acidic glycoprotein. It can be emphasized that targeting can tether the dipyridamole group to the cell surface and result in extremely high effective concentrations at the nucleoside transporter sites at the site of action on the cell surface. The following references relate to this subject matter: Baldwin S. A., et al., “Nucleoside Transporters: Molecular Biology and Implications for Therapeutic Development,” Molecular Med Today, 5:216-224 (1999); Bamford C. H., et al., “Polymeric Inhibitors of Platelet Aggregation. II. Copolymers of Dipyridamole and Related Drugs with N-vinylpyrrolidone,” Biochimica et Biophysica Acta, 924:38-44 (1987), the contents of which are incorporated herein by reference in their entirety.

Or E2 comprises the structure below referred to as embodiment 1 E2.3:

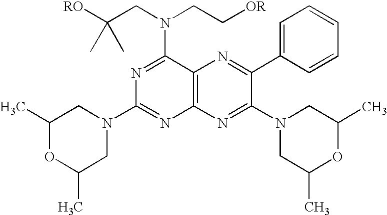

wherein R is H or a bioreversible hydroxy masking group that undergoes spontaneous or enzymatically triggered cleavage to expose the free hydroxy moiety; or wherein R is the site of linker attachment to the remainder of ET. This structure is based upon the ability of compound BIBW 22 to inhibit nucleoside transport. The following references relate to this subject matter: Chen H., et al., “BIBW 22, a Dipyridamole Analogue, Acts as a Bifunctional Modulator on Tumor Cells by Influencing Both P-Glycoprotein and Nucleoside Transport,” Cancer Research, 53:1974-1977 (1993), the contents of which are incorporated herein by reference in their entirety.

Or E2 comprises the following structure (referred to as embodiment 1E2.4),

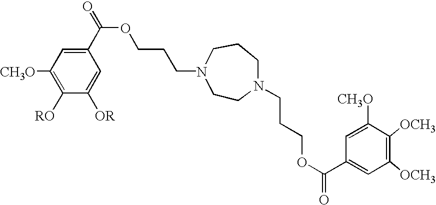

which is based upon the ability of dilazep to inhibit nucleoside transport.

Wherein R is H or a bioreversible hydroxy masking group that undergoes spontaneous or enzymatically triggered cleavage to expose the free hydroxy moiety or wherein R is the site of linker attachment to the remainder of ET.

Another preferred embodiment is based on the super synergystic toxicity that results from the combination of folic acid, inhibitors of dihydrofolate reductase, and inhibitors of other folate dependent enzymes such as glycinamide ribonucleotide formyltransferase, 5-aminomidazole-4-carboxamide ribonucleotide formyltransferase, and thymidylate synthase. The mechanisms responsible for this super synergystic toxicity are poorly understood. The following references relate to this subject matter: Gaumont Y., et al., “Quantitation of Folic Acid Enhancement of Antifolate Synergism,” Cancer Research, 52:2228-2235 (1992); Faessel H. M., et al., “Super in Vitro Synergy between Inhibitors of Dihydrofolate Reductase and Inhibitors of other Folate-requiring Enzymes: The Critical Role of Polyglutamylation,” Cancer Research, 58:3036-3050 (1998.); Kisliuk R. L., et al., “The Effect of Polyglutamylation on the Inhibitory Activity of Folate Analogs,” In: D.Goldman (ed,), Proceedings of the Second Workshop on Folyl and Antifolyl Polyglutamates, pp. 319-328. New York: Praeger (1985); Kisliuk R. L., et al., “Synergistic Growth Inhibition by Combination of Antifolates,” In: M. F. Picciano, et al., (eds.), Evaluation of Folate Metabolism in Health and Disease, pp. 79-89, New York: Alan R. Liss (1990); Galivan J., et al., “Antifolate Drug Interactions: Enhancement of Growth Inhibition Due to the Antipurine 5,10-Dideazatetrahydrofolic Acid by the Lipophilic Dihydrofolate Reductase Inhibitors Metoprine and Trimetrexate,” Cancer Res, 48:2421-2425 (1988); Galivan J., et al., “Synergistic Growth Inhibition of Rat Hepatoma Cells Exposed in Vitro to N¹⁰-Propargyl-5,8-dideazafolate with Methotrexate or the Lipophilic Antifolates Trimetrexate or Metoprine,” Cancer Res, 47:5256-5260 (1987); Faessel H. M., et al., “Folic Acid-enhanced Synergy for the Combination of Trimetrexate Plus the Glycinamide Ribonucleotide Formyltransferase Inhibitor 4-[2-(2-amino-4-oxo-4,6,7,8-tetrahydro-3H-pyrimidino[5,4,6][1,4]thiazin-6-yl)-(S)-ethyl]-2,5-thienoylamino-L-glutamic Acid (AG2034): Comparison Across Sensitive and Resistant Human Tumor Cell Lines,” Biochem Pharmacol, 57(5):567-77 (1999); Galivan J., et al., “The Role of Cellular Folates in the Enhancement of Activity of the Thymidylate Synthase Inhibitor 10-Propargyl-5,8-dideazafolate against Hepatoma Cells in Vitro by Inhibitors of Dihydrofolate Reductase,” J Biological Chem, 264(18):10685-10692 (1989), the contents of which are incorporated herein by reference in their entirety.

A preferred embodiment of the present invention consists of the set of targeted drugs E1-T1 and E2-T2, wherein E1 comprises an inhibitor of dihydrofolate reductase and E2 comprises an inhibitor of glycinamide ribonucleotide formyltransferase, 5-aminomidazole-4-carboxamide ribonucleotide formyltransferase, or thymidylate synthase, and folic acid is administer in conjunction with the targeted drugs E1-T1 and E2-T2.

In preferred embodiments, E1 comprises the structure (2E1.1) shown below:

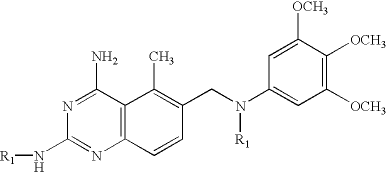

wherein R₁ is H, or a bioreversible amino masking group which when triggered by enzymatic or spontaneous processes cleaves the R₁—N bond and wherein R₁ can also bear a site of linker attachment to the remainder of the drug complex. Activation of the trigger can liberate the dihydrofolate reductase inhibitor trimetrexate.

and E2 comprises the structure (2E2.1) shown below:

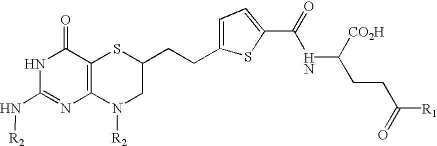

wherein R₁ is OH, or the site of linker attachment to the remainder of the drug complex, and R₂ is H, or a bioreversible amino protecting group which when triggered by enzymatic or spontaneous mechanisms unmasks the free amino group, and where R2 can also bear a site of linker attachment to the remainder of ET complex.

The above structure is based on AG2034 a compound that is a potent inhibitor of glycinamide ribonucleotide formyltransferase. The masking group R₂ can be used to prevent binding to the folate receptor from defining the domain of targeting specificity. The following references relate to this subject matter: Varney M. D., et al., “Protein Structure-Based Design, Synthesis, and Biological Evaluation of 5-Thia-2,6-diamino-4(3H)-oxopyrimidines: Potent Inhibitors of Glycinamide Ribonucleotide Transformylase with Potent Cell Growth Inhibition,” J Med Chem, 40:2502-2524 (1997); Boritzki T. J., et al., “AG2034: A Novel Inhibitor of Glycinamide Ribonucleotide Formyltransferase,” Invest New Drugs, 14(3):295-303 (1996), the contents of which are incorporated herein by reference in their entirety.

Or E2 comprises the structure (2E2.2) shown below:

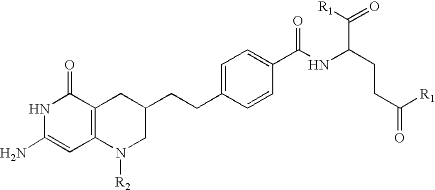

wherein R₁ is OH, or the site of linker attachment to the remainder of the drug complex; or wherein R₁ can be a bioreversible protecting group which when triggered unmasks the carboxylate group and to which is attached a linker connnected to the remainder of the drug complex, and R₂ is H, or a bioreversible amino protecting group which when triggered by enzymatic or spontaneous mechanisms unmasks the free amino group. This structure is based on Lometrexol a compound that is a potent inhibitor of glycinamide ribonucleotide formyltransferase. The following references relate to this subject matter: Roberts J. D., et al., “Weekly Lometrexol with Daily Oral Folic Acid is Appropriate for Phase II Evaluation,” Cancer Chemother Pharmacol, 45(2):103-10 (2000), the contents of which are incorporated herein by reference in their entirety.

Or E2 comprises the structure (2E2.3) shown below:

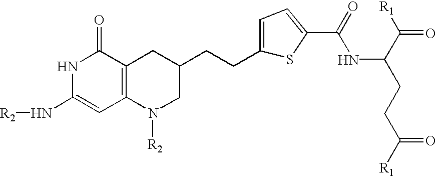

wherein R₁ is OH, or the site of linker attachment to the remainder of the drug complex; or wherein R₁ can be a bioreversible protecting group which when triggered unmasks the carboxylate group and to which is attached a linker connnected to the remainder of the drug complex, and R₂ is H, or a bioreversible amino protecting group which when triggered by enzymatic or spontaneous mechanisms unmasks the free amino group; and wherein R₂ can have a site of linker attachment to the remainder of ET. This structure is based on LY309887 a compound that is a potent inhibitor of glycinamide ribonucleotide formyltransferase. The following references relate to this subject matter: Mendelsohn L. G., et al., “Biochemistry and Pharmacology of Glycinamide Ribonucleotide Formyltransferase Inhibitors: LY309887 and Lometrexol,” Invest New Drugs, 14(3):287-94 (1996), the contents of which are incorporated herein by reference in their entirety.

Or E2 comprises the structure (2E2.3) shown below::

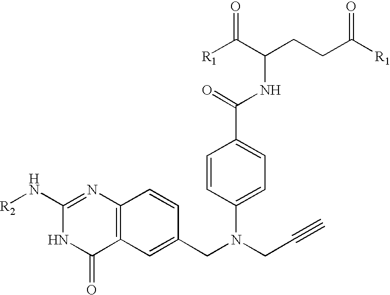

wherein R₁ is OH, or the site of linker attachment to the remainder of the drug complex; or wherein R₁ can be a bioreversible protecting group which when triggered unmasks the carboxylate group and to which is attached a linker connnected to the remainder of the drug complex, and R₂ is H, or a bioreversible amino protecting group which when triggered by enzymatic or spontaneous mechanisms unmasks the free amino group; and wherein R₂ can have a site of linker attachment to the remainder of the drug complex. This structure is based on 10-propargyl-5-8-dideazafolic acid, a potent inhibitor of thymidylate synthase.

In a preferred embodiment the above are E1-T1 and E2-T2 are administered in conjunction with folic acid.

Another preferred embodiment is based on the synergystic toxicity that results from the inhibition of denovo guanine nucleotide synthesis and the salvage pathway by inhibition of hypoxanthine-guanine phosphoribosyltransferase. The following references relate to this subject matter: Weber G., et al., “Regulation of De Novo and Salvage Pathways in Chemotherapy,” Adv Enzyme Regul, 31:45-67 (1991); Weber G., et al., “Salvage Capacity of Hepatoma 3924A and Action of Dipyridamole”, Adv Enzyme Regul, 21:53-69 (1983); Digits J. A.; Hedstrom L., “Species-Specific Inhibition of Inosine 5′-Monophosphate Dehydrogenase by Mycophenolic Acid,” Biochemistry, 38:15388-15397 (1999), the contents of which are incorporated herein by reference in their entirety.

A preferred embodiment of the present invention consists of the set of targeted drugs E1-T1 and E2-T2, wherein E1 comprises an inhibitor to inosine monophosphate dehydrogenase and E2 comprises an inhibitor to hypoxanthine-guanine phosphoribosyltransferase.

In a preferred embodiment, E1 comprises the following structure referred to embodiment 3E1.1:

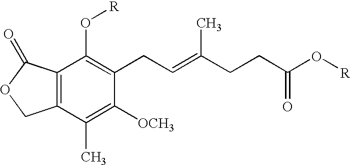

wherein R is H, or a bioreversible masking group which when triggered by enzymatic or chemical processes exposes the free OH group, and wherein R can have a site of linker attachment to the remainder of the drug complex. This structure is based on mycophenolic acid, which inhibits IMP dehydrogenase at nanomolar levels. The following references relate to this subject matter: Shi W., et al., “The 2.0 A Structure of Human Hypoxanthine-guanine Phosphoribosyltransferase in Complex with a Transition-state Analog Inhibitor,” Nature Structural Biology, 6(6):588-593 (1999); Digits J. A.; Hedstrom L., “Species-Specific Inhibition of Inosine 5′-Monophosphate Dehydrogenase by Mycophenolic Acid,” Biochemistry, 38:15388-15397 (1999), the contents of which are incorporated herein by reference in their entirety.

And, E2 comprises the structure referred to as 3E2.1:

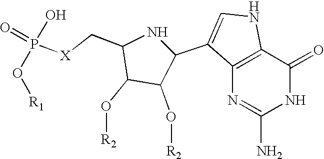

wherein R₁ and R₂ is H, or a bioreversible masking group which when triggered by enzymatic or chemical processes exposes the free OH group; and wherein R₁ or R₂ can can have a site of linker attachment to the remainder of ET; and wherein X is O or CH₂. This structure is based on immucillin GP, which inhibits hypoxanthine-guanine phosphoribosyltransferase at low nonmolar levels. The following references relate to this subject matter: Shi W., et al., “The 2.0 A Structure of Human Hypoxanthine-guanine Phosphoribosyltransferase in Complex with a Transition-state Analog Inhibitor,” Nature Structural Biology, 6(6):588-593 (1999), the contents of which are incorporated herein by reference in their entirety.

Or E1 can comprise the following structure (3E1.2):

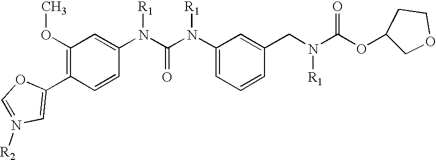

wherein R₁ is H or a bioreversible masking group which when triggered cleaves the R—N bond, and wherein R₁ or R₂ can have a site of linker attachment to the remainder of ET; and wherein R₂ can be a bioreversible masking group which when triggered cleaves the R—N bond, or wherein R₂ can be absent from the structure. Activation of the trigger can release VX-497, which is a potent inhibitor of IMP dehydrogenase. The following references relate to this subject matter: Markland W., et al., “Broad-Spectrum Antiviral Activity of the IMP Dehydrogenase Inhibitor VX-497: a Comparison with Ribavirin and Demonstration of Antiviral Additivity with Alpha Interferon,” Antimicrobial Agents and Chemotherapy, 44(4):859-866 (2000), the contents of which are incorporated herein by reference in their entirety.

Additional synergystic toxicity would be expected upon the addition of a third drug E3-T3 in which E3 is a nucleoside transport inhibitor as described above as embodiments 1E2.1,1 E2.2, 1E2.3 and 1E2.4.

A preferred embodiment of the present invention consists of the set of targeted drugs E1-T1 and E2-T2, wherein E1 comprises an inhibitor to dihydroorotic acid dehydrogenase and E2 comprises an inhibitor to nucleoside transport. Dihydroorotic acid dehydrogenase is the fourth enzyme in the committed pathway of de novo pyrimidine synthesis. A preferred embodiment is based on brequinar, a compound that inhibits dihydroorotic acid dehydrogenase at nonamolar levels. The following references relate to this subject matter: Bruneau J. M., et al., “Purification of Human Dihydro-orotate Dehydrogenase and its Inhibition by A77 1726, The Active Metabolite of Leflunomide,” Biochem J, 336, 299-303 (1998); Chen S. F., et al., “Inhibition of Dihydroorotate Dehydrogenase Activity by Brequinar Sodium,” Cancer Res, 52:3521-3527 (1992), the contents of which are incorporated herein by reference in their entirety.

In a preferred embodiment, E1 comprises the following structure (4E1.1):

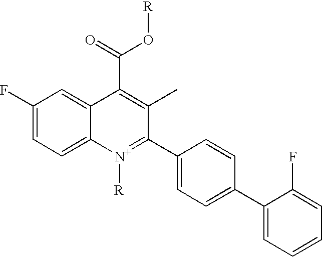

wherein R is H or the site of bioreversible masking group to which is attached a linker connected to the remainder of the drug complex, wherein spontaneous or enzymatic triggering unmasks the the active enzyme inhibitor.

And, E2 is comprised of an inhibitor to nucleoside transport such as described previously in embodiments 1E2.1, 1 E2.2, 1E2.3 and 1E2.4.

A preferred embodiment of the present invention consists of the set of targeted drugs E1-T1 and E2-T2, wherein E1 comprises an inhibitor to orotidine 5′-phosphate decarboxylase and E2 comprises an inhibitor to nucleoside transport. Orotidine 5′-phosphate decarboxylase catalyzes the final step in the de novo synthesis of uridine monophosphate. A preferred embodiment is based upon 1-(5′-phospho-ribofuranosyl)barbituric acid which is a potent inhibitor of the enzyme. The following references relate to this subject matter: Levine H. L., et al., “Inhibition of Orotidine-5′-phosphate Decarboxylase by 1-(5′-Phospho-β-D-ribofuranosyl)barbituric Acid, 6-Azauridine 5′-Phosphate, and Uridine 5′-Phosphate,” Biochemistry, 19:4993-4999 (1980), the contents of which are incorporated herein by reference in their entirety.

In a preferred embodiment, E1 comprises the following structure (5E1.1):

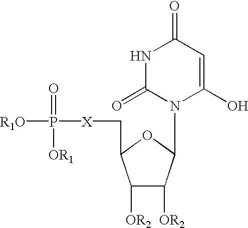

wherein in X is O, CH₂, or S, and R₁ is H, or a bioreversible phosphate protecting group which when triggered by spontaneous or enzymatic processes unmasks the free phosphate; and in which R₁ can have a site of linker attachment to the remainder of ET; and wherein R₂ is H or a bioreversible hydroxy protecting group which when activated unmasks the free hydroxy group and wherein R₂ can have a site of linker attachment to the remainder of ET.

And, E2 is comprised of an inhibitor to nucleoside transport such as described previously in embodiments 1E2.1, 1E2.2, 1E2.3 and 1E2.4.

In a preferred embodiment, E1 comprises an inhibitor of aspartate transcarbamylase, the second key enzyme in the de novo synthesis of pyrimidine rings. A preferred embodiment is based on (N-phosphonoacetyl)-L-aspartate, which is a potent inhibitor of aspartate transcarbamylase. The following references relate to this subject matter: Erlichman C., “An Overview of the Clinical Pharmacology of N-phosphonacetyl-L-aspartate (PALA), a New Antimetabolite,” Recent Results Cancer Res, 74:65-71 (1980); Johnson R. K., et al., “Antitumor Activity of N-(phosphonacetyl)-L-aspartic Acid, a Transition-State Inhibitor of Aspartate Transcarbamylase,” Cancer Res, 36(8):2720-5 (1976); Erlichman C.; Vidgen D., “Antitumor Activity of N-phosphonacetyl-L-aspartic Acid in Combination with Nitrobenzylthioinosine,” Biochem Pharmacol, 33(20):3177-81 (1984), the contents of which are incorporated herein by reference in their entirety.

In a preferred embodiment, E1 comprises the following structure (6E1.1):

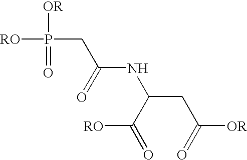

Wherein R is H, or a bioreversible protecting group which when triggered by spontaneous or enzymatic processes unmasks the free phosphonate or carboxylate group, and in which R can have a site of linker attachment to the remainder of ET.

And, E2 is comprised of an inhibitor to nucleoside transport such as described previously in embodiments 1E2.1,1 E2.2, 1E2.3 and 1E2.4

Methods of Use

The compounds of the present invention are used by contacting the target cells with a sufficient quantity to evoke the desired diagnostic or therapeutic result. The drugs can be administered in combination with commonly employed pharmacological excipients, preservatives and stabilizers that are well known to one skilled in the arts. In general, the drugs are for intravenous use and can be administered dissolved in sterile saline or water or a buffered salt solution. In selected situations the drugs could be given routes such as intra-arterially, intraperitoneally, orally or topically.

The drugs should be administered to a patient in a sufficient amount and for a sufficient period of time to achieve the desired pharmacological result and will depend upon the severity of the illness and the other factor well known to one skilled in the art. For a drug ET in which E is comprised of a known drug, the dose of ET can be lower than or about equal to the dose of drug E as currently used in clinical practice. The dose of the drug administered can be in the range of about 1 picogram per kilogram body weight to about 50 mg/kg.

In a preferred embodiment the drugs ET are administered at ultra-low dose as described below. In other embodiments the drug ET is given at conventional doses similar to those currently used for the drug E. Procedures for dose optimization are well known to one skilled in the art.

For diagnostic use, routine procedures and methodologies applicable to the detection and imaging of the targeted moiety can be used.

Anti-cancer Therapy

Targeted Toxins

The following general guidelines and principles are relevant to the use of anti-cancer drugs of the class described herein.

1.) The smallest dose of drug that exceeds that required to saturate the target receptors on the tumor cells can be used. This is referred to, in this patent application, as “ultra-low dose”. This can be a dose that results in subnanomolar to picomolar plasma concentrations or can be higher depending upon the affinity of the particular drug for the tumor cells. The use of excess drug dosage can lower the targeting selectivity and therapeutic index without increasing therapeutic efficacy.

2.) Only a subset of tumor cells at any given time can be able to contact the drug. Tumors are heterogeneous with respect to drug penetration even for small molecules. Accordingly, multiple cycles of therapy can be used.

3.) Only a subset of the tumor cells can be sensitive to any particular drug. There is no point to single agent therapy. Accordingly, multiple drugs can be used concurrently. The extremely low doses employed can allow for the simultaneous administration of effective doses of multiple targeted agents without prohibitive toxicity.

4.) When applicable, the drugs can be used in conjunction with agents that suppress delivery to non-tumor areas. For example, a drug, which inclus a targeting ligand against glutamate carboxypeptidase II for the treatment of prostate or breast cancer, can be used in conjunction with an orally administered nonabsorbable inhibitor to the enzyme to suppress targeting to the enzyme on the luminal surface of the small intestine.

5.) If a particular targeted drug has significant nonspecific affinity to serum proteins then it is advisable to administer a pharmaceutical agent, which competitively displaces the targeted drug (displacer drug) from the serum protein. Conceptually, this is similar to the displacement of phenytoin by salicylate from serum albumen. Since the displacer drug can be selected to be of very low toxicity, concentrations thousands of times higher then the target drug can be employed to give effective competitive inhibition of the nonspecific protein binding.

6.) The targeted toxin class of drugs can be used in conjunction with targeted drugs that stimulate the innate or adaptive immune system. These drugs can provoke an inflammatory reaction at the tumor site. These targeted drugs can be given first, and then after about 48 hours when a tumor inflammatory reaction is present, the targeted toxin type drugs can be administered. The inflammatory reaction can facilitate the tumor penetration of the drugs. Targeted toxin type drugs may also be given concurrently with targeted immunostimulator type drugs.

7.) If the drug has a detoxifying trigger that is activated by an independently targeted antibody enzyme conjugate then the drug can be administered after the detoxifying enzyme has localized to the non-tumor cells.

8.) If the drug has a tumor-selective trigger, that can be activated by an enzyme independently targeted to tumor cells, then the drug can be administered first, allowed to localize to target cells, and then the targeted enzyme trigger administered.

9.) If the drug bears a masked transporter ligand comprised of masked biotin then the drug can be administered, allowed to localize to target cells, and then the avidin-transporter moieties can be administered.

10.)The drugs can be used in addition to other anti-cancer therapeutic modalities such as surgery, radiation therapy, angiogenisis inhibitors, and immunotherapy.

11.)Tumor cells develop resistance to drugs by predictable “escape mechanisms”. The typical response of tumors to a metabolic inhibitor is a compensatory increase in the expression of the targeted enzyme and increased expression of enzymes that by-pass the inhibited metabolic step. By the administration of a combination of drugs these “escape mechanisms” can be transformed into an ever-tightening noose which amplifies tumor killing. The use of a metabolic inhibitor, coupled with targeting, directed against the mechanisms of resistance to that inhibitor, can increase tumor killing cells by the very mechanisms that typically confer drug resistance.

Ultra-low Dose Multiple Drug Multiple Target Therapy

It is increasingly apparent that cancer is not a single disease, but a changing spectrum of different diseases even in an individual patient. The average colon cancer cell has over ten thousand different DNA mutations. The following references relate to this subject matter: Stoler D. L., et al., “The Onset and Extent of Genomic Instability in Sporadic Colorectal Tumor Progression,” PNAS, 96(26):15121-15126 (1999), the contents of which are incorporated herein by reference in their entirety.

A patient with disseminated cancer can have 1 trillion (10¹²) cancer cells spread throughout the body. To ensure eradication of the disease, it is necessary to kill every last cancer cell without undo toxicity to the patient. Any single drug against any single tumor target can give at most a 2 to 4 log reduction in tumor cell burden, which represents killing of 99% to 99.99% of the tumor cells. Potentially any one of the residual tumor cells can grow and cause progressive illness from cancer. The only way to deal with this is to use multiple independent drugs or therapies directed against multiple tumor targets. If the probability that a tumor cell can develop resistance to a single drug is 10⁻² then the joint probability that a tumor cell could develop resistance simultaneously to 10 independent drugs is 10^{-20.} In other words, the combination of ten independent drugs can be a billion billion times more effective than a single drug.

Today it is difficult to treat cancer patients with even one or two anti-cancer drugs at a time, because the drugs are poorly selective and highly toxic. A severe price is paid if a patient's tumor is resistant to the anti-cancer drugs. Toxicity often precludes the administration of effective doses of alternate drugs. What is needed is a failure tolerant anti-cancer technology based on the reality that no single drug can be effective. What is needed is a technology to enable the use of multiple drugs against multiple targets so that the probability of tumor escape is precluded.

The technology detailed in this patent application is designed to enable the simultaneous administration of multiple drugs against multiple tumor targets without undo toxicity. The high binding affinity and selectivity that multifunctional drug delivery vehicles can have for tumor cells can translate into the ability to effectively target tumor cells with ultra-low nontoxic doses of drugs.

The present invention is a method of treating cancer that is comprised of the administration of ultra-low doses of multiple drugs targeted against multiple properties of the tumor. The definition of “ultra-low dose” was previously given. The drugs can be given simultaneously or in sufficient temporal proximity that for resistance to develop, the tumor cells must acquire joint resistance to each agent. The method of ultra-low dose multiple drug multiple target therapy is by its very design inherently failure tolerant. The key is redundancy of targeting and mechanisms of tumor cell killing. In this method the average tumor cell can be exposed to numerous (about 2 to about 20) different drugs, any single one of which would be sufficient to kill the tumor cell. Although this can seem like massive over-kill reminiscent of the nuclear arms race, this is what is realistically needed to address the problem of cancer. Killing the average cancer cell is clinically meaningless. What is needed is to kill the last cancer cell. Tumor heterogeneity mandates the use of multiple drugs against multiple targets to achieve this goal.

Extremely minute quantities of anti-cancer drugs when delivered into cancer cells can be lethal to the cell. For example, 500 molecules of bleomycin delivered intracellularly are sufficient to kill the cell. The following references relate to this subject matter: Pron G., et al., “Internalisation of the Bleomycin Molecules Responsible for Bleomycin Toxicity: A Receptor-mediated Endocytosis Mechanism,” Biochemical Pharmacology, 57:45-56 (1999), the contents of which are incorporated herein by reference in their entirety.

In principal, given ideal drug delivery, a patient with widely disseminated cancer and a tumor burden of 100 billion cancer cells could be treated with 5×10¹³ molecules or approximately 10⁻¹⁰ moles of drug. For a 100 kg patient this represents a drug concentration of about 10⁻¹² Molar. The drugs embodied by the present invention are designed to approach this ideal, but in practice unachievable, theoretical limit of minimal drug dose. Currently in actual practice patients are treated with bleomycin at doses approximately 100,000 times higher.

The method of ultra-low dose multiple drug multiple target therapy is based upon the ability of multifunctional drug delivery vehicles to selectively deliver and trap cytotoxic concentrations of drug inside tumor cells at doses far below levels which can produce systemic toxicity. The tighter the binding affinity the lower the drug concentration that is required to saturate the target receptors on the tumor cells and deliver a lethal dose of drug to the tumor cells. The drugs embodied by the present invention are expected to bind effectively to tumor cells at concentrations that are orders of magnitude lower than the levels needed to produce systemic toxicity.

A preferred embodiment consists of administering to a patient with cancer the drugs (E1-T1), (E2-T2) and . . . (En-Tn); which are compounds of the present invention; and wherein the drugs are directed against or selective for multiple sets of targets that are increased on tumor cells; and wherein the drugs deliver multiple different antitumor agents. Preferably the delivered effector agents should be such that tumor resistance develops by different independent mechanisms for each drug. The drugs are administered systemically for a sufficient duration, at a sufficient dose, and sufficient frequency to achieve the desired antitumor response.

In a preferred embodiment of the above, the doses are ultra-low wherein ultra-low refers to a minimal dose that is sufficient to bind the drug to target receptors on accessible tumor cells. An accessible tumor cell is a tumor cell that is able to contact the drug.

Targeted Masked Antigens and Targeted Neoantigens

Drugs, which exert activity by evoking an immune response to a targeted masked antigen or a targeted neoantigen, require that the patient be pre-sensitized to the relevant antigens prior to drug therapy. This can be accomplished by immunizing the patient with the respective unmasked antigen or neoantigen in combination with a variety of adjuvants and immunostimulators. The antigen can be administered by a variety of routes with the intradermal route being preferred. Only that portion of the drug bearing the antigenic moiety or the neoantigen is used for immunization purposes. In some cases, it can be desirable to use an antigenic moiety with a short linker bearing a reactive group such as an isothiocyanate group. The function of this group is to increase the immunogenicity of the antigen by enhancing uptake and presentation by dendritic cells. As discussed previously, the sensitization can also be conducted in vitro and adoptively transferred by the infusion of sensitized lymphocytes.

The drugs of the present invention may be given to either a person or an animal in need of the pharmaceutical effect of said drugs.

Additional Preferred Embodiments of the Invention:

Preferred Combinations of Tumor Targeting Ligands

In Preferred Embodiments;

designated: (embodiment TLP #.X, wherein X is the number given below to the pairs of target receptors and X=1, 2, 3, . . . 795);

ET is an anti-cancer drug or diagnostic drug comprised of one targeting ligand that binds the first target receptor (a1) and a second targeting ligand that binds to the second target receptor (a2) indicated in the pairs of (a1 - - - a2) listed below:

1) urokinase—a cathepsin type protease;

2 urokinase—a collagenase;

3) urokinase—a gelatinase;

4) urokinase—a matrix metalloproteinase;

5) urokinase—a membrane type matrix metalloproteinase;

6) urokinase—alpha v beta 3 integrin;

7) urokinase—bombesin/gastrin releasing peptide receptors;

8) urokinase—cathepsin B;

9) urokinase—cathepsin D;

10) urokinase—to cathepsin K;

11) urokinase—cathepsin L;

12) urokinase—cathepsin O;

13) urokinase—fibroblast activation protein;

14) urokinase—folate binding receptors;

15) urokinase—gastrin/cholecystokinin type B receptor;

16) urokinase—glutamate carboxypeptidase II or (PSMA);

17) urokinase—guanidinobenzoatase;

18) urokinase—laminin receptor;

19) urokinase—matrilysin;

20) urokinase—matripase;

21) urokinase—melanocyte stimulating hormone receptor;

22) urokinase—nitrobenzylthioinosine-binding receptors or (nucleoside transporter);

23) urokinase—norepinephrine transporters;

24) urokinase—nucleoside transporter proteins;

25) urokinase—peripheral benzodiazepam binding receptors;

26) urokinase—plasmin;

27) urokinase—seprase;

28) urokinase—sigma receptors;

29) urokinase—somatostatin receptors;

30) urokinase—stromelysin 3;

31) urokinase—trypsin;

32) urokinase—urokinase;

33) urokinase—MMP 1;

34) urokinase—MMP 2;

35) urokinase—MMP 3;

36) urokinase—MMP 7;

37) urokinase—MMP 9;

38) urokinase—membrane type matrix metalloproteinase I;

39) urokinase—MMP 12;

40) urokinase—MMP 13;

41) urokinase—a tumor antigen;

42) plasmin—a cathepsin type protease;

43) plasmin—a collagenase;

44) plasmin—a gelatinase;

45) plasmin—a matrix metalloproteinase;

46) plasmin—a membrane type matrix metalloproteinase;

47) plasmin—alpha v beta 3 integrin;

48) plasmin—bombesin/gastrin releasing peptide receptors;

49) plasmin—cathepsin B;

50) plasmin—cathepsin D;

51) plasmin—to cathepsin K;

52) plasmin—cathepsin L;

53) plasmin—cathepsin O;

54) plasmin—fibroblast activation protein;

55) plasmin—folate binding receptors;

56) plasmin—gastrin/cholecystokinin type B receptor;

57) plasmin—glutamate carboxypeptidase II or (PSMA);

58) plasmin—guanidinobenzoatase;

59) plasmin—laminin receptor;

60) plasmin—matrilysin;

61) plasmin—matripase;

62) plasmin—melanocyte stimulating hormone receptor;

63) plasmin—nitrobenzylthioinosine-binding receptors or (nucleoside transporter);

64) plasmin—norepinephrine transporters;

65) plasmin—nucleoside transporter proteins;

66) plasmin—peripheral benzodiazepam binding receptors;

67) plasmin—plasmin;

68) plasmin—seprase;

69) plasmin—sigma receptors;

70) plasmin—somatostatin receptors;

71) plasmin—stromelysin 3;

72) plasmin—trypsin;

73) plasmin—urokinase;

74) plasmin—MMP 1;

75) plasmin—MMP 2;

76) plasmin—MMP3;

77) plasmin—MMP 7;

78) plasmin—MMP 9;

79) plasmin—membrane type matrix metalloproteinase I;

80) plasmin—MMP 12;

81) plasmin—MMP 13;

82) plasmin—a tumor antigen;

83) a collagenase—a cathepsin type protease;

84) a collagenase—a collagenase;

85) a collagenase—a gelatinase;

86) a collagenase—a matrix metalloproteinase;

87) a collagenase—a membrane type matrix metalloproteinase;

88) a collagenase—alpha v beta 3 integrin;

89) a collagenase—bombesin /gastrin releasing peptide receptors;

90) a collagenase—cathepsin B;

91) a collagenase—cathepsin D;

92) a collagenase—to cathepsin K;

93) a collagenase—cathepsin L;

94) a collagenase—cathepsin O;

95) a collagenase—fibroblast activation protein;

96) a collagenase—folate binding receptors;

97) a collagenase—gastrin/cholecystokinin type B receptor;

98) a collagenase—glutamate carboxypeptidase II or (PSMA);

99) a collagenase—guanidinobenzoatase;

100) a collagenase—laminin receptor;

101) a collagenase—matrilysin;

102) a collagenase—matripase;

103) a collagenase—melanocyte stimulating hormone receptor;

104) a collagenase—nitrobenzylthioinosine-binding receptors or (nucleoside transporter);

105) a collagenase—norepinephrine transporters;

106) a collagenase—nucleoside transporter proteins;

107) a collagenase—peripheral benzodiazepam binding receptors;

108) a collagenase—seprase;

109) a collagenase—sigma receptors;

110) a collagenase—somatostatin receptors;

111) a collagenase—stromelysin 3;

112) a collagenase—trypsin;

113) a collagenase—a collagenase;

114) a collagenase—MMP 1;

115) a collagenase—MMP 2;

116) a collagenase—MMP 3;

117) a collagenase—MMP 7;

118) a collagenase—MMP 9;

119) a collagenase—membrane type matrix metalloproteinase I;

120) a collagenase—MMP 12;

121) a collagenase—MMP 13;

122) a collagenase—a tumor antigen;

123) a gelatinase—a cathepsin type protease;

124) a gelatinase—a gelatinase;

125) a gelatinase—a matrix metalloproteinase;

126) a gelatinase—a membrane type matrix metalloproteinase;

127) a gelatinase—alpha v beta 3 integrin;

128) a gelatinase—bombesin/gastrin releasing peptide receptors;

129) a gelatinase—cathepsin B;

130) a gelatinase—cathepsin D;

131) a gelatinase—to cathepsin K;

132) a gelatinase—cathepsin L;

133) a gelatinase—cathepsin O;

134) a gelatinase—fibroblast activation protein;

135) a gelatinase—folate binding receptors;

136) a gelatinase—gastrin/cholecystokinin type B receptor;

137) a gelatinase—glutamate carboxypeptidase II or (PSMA);

138) a gelatinase—guanidinobenzoatase;

139) a gelatinase—laminin receptor;

140) a gelatinase—matrilysin;

141) a gelatinase—matripase;

142) a gelatinase—melanocyte stimulating hormone receptor;

143) a gelatinase—nitrobenzylthioinosine-binding receptors or (nucleoside transporter);

144) a gelatinase—norepinephrine transporters;

145) a gelatinase—nucleoside transporter proteins;

146) a gelatinase—peripheral benzodiazepam binding receptors;

147) a gelatinase—seprase;

148) a gelatinase—sigma receptors;

149) a gelatinase—somatostatin receptors;

150) a gelatinase—stromelysin 3;

151) a gelatinase—trypsin;

152) a gelatinase—MMP 1;

153) a gelatinase—MMP 2;

154) a gelatinase—MMP 3;

155) a gelatinase—MMP 7;

156) a gelatinase—MMP 9;

157) a gelatinase—membrane type matrix metalloproteinase I;

158) a gelatinase—MMP 12;

159) a gelatinase—MMP 13;

160) a gelatinase—a tumor antigen;

161) a matrix metalloproteinase—a cathepsin type protease;

162) a matrix metalloproteinase—a collagenase;

163) a matrix metalloproteinase—a gelatinase;

164) a matrix metalloproteinase—a matrix metalloproteinase;

165) a matrix metalloproteinase—a membrane type matrix metalloproteinase;

166) a matrix metalloproteinase—alpha v beta 3 integrin;

167) a matrix metalloproteinase—bombesin/gastrin releasing peptide receptors;

168) a matrix metalloproteinase—cathepsin B;

169) a matrix metalloproteinase—cathepsin D;

170) a matrix metalloproteinase—to cathepsin K;

171) a matrix metalloproteinase—cathepsin L;

172) a matrix metalloproteinase—cathepsin O;

173) a matrix metalloproteinase—fibroblast activation protein;

174) a matrix metalloproteinase—folate binding receptors;

175) a matrix metalloproteinase—gastrin/cholecystokinin type B receptor;

176) a matrix metalloproteinase—glutamate carboxypeptidase II or (PSMA);

177) a matrix metalloproteinase—guanidinobenzoatase;

178) a matrix metalloproteinase—laminin receptor;

179) a matrix metalloproteinase—matrilysin;

180) a matrix metalloproteinase—matripase;

181) a matrix metalloproteinase—melanocyte stimulating hormone receptor;

182) a matrix metalloproteinase—nitrobenzylthioinosine-binding receptors or (nucleoside transporter);

183) a matrix metalloproteinase—norepinephrine transporters;

184) a matrix metalloproteinase—nucleoside transporter proteins;

185) a matrix metalloproteinase—peripheral benzodiazepam binding receptors;

186) a matrix metalloproteinase—plasmin;

187) a matrix metalloproteinase—seprase;

188) a matrix metalloproteinase—sigma receptors;

189) a matrix metalloproteinase—somatostatin receptors;

190) a matrix metalloproteinase—stromelysin 3;

191) a matrix metalloproteinase—trypsin;

192) a matrix metalloproteinase—a matrix metalloproteinase;

193) a matrix metalloproteinase—MMP 1;

194) a matrix metalloproteinase—MMP 2;

195) a matrix metalloproteinase—MMP 3;

196) a matrix metalloproteinase—MMP 7;

197) a matrix metalloproteinase—MMP 9;

198) a matrix metalloproteinase—membrane type matrix metalloproteinase I;

199) a matrix metalloproteinase—MMP 12;

200) a matrix metalloproteinase—MMP 13;

201) a matrix metalloproteinase—a tumor antigen;

202) a membrane type metalloproteinase—a cathepsin type protease;

203) a membrane type metalloproteinase—a membrane type matrix metalloproteinase;

204) a membrane type metalloproteinase—alpha v beta 3 integrin;

205) a membrane type metalloproteinase—bombesin/gastrin

releasing peptide receptors;

206) a membrane type metalloproteinase—cathepsin B;

207) a membrane type metalloproteinase—cathepsin D;

208) a membrane type metalloproteinase—to cathepsin K;

209) a membrane type metalloproteinase—cathepsin L;

210) a membrane type metalloproteinase—cathepsin 0;

211) a membrane type metalloproteinase—fibroblast activation protein;

212) a membrane type metalloproteinase—folate binding receptors;

213) a membrane type metalloproteinase—gastrin/cholecystokinin type B receptor;

214) a membrane type metalloproteinase—glutamate carboxypeptidase II or (PSMA);

215) a membrane type metalloproteinase—guanidinobenzoatase;

216) a membrane type metalloproteinase—laminin receptor;

217) a membrane type metalloproteinase—matrilysin;

218) a membrane type metalloproteinase—matripase;

219) a membrane type metalloproteinase—melanocyte stimulating hormone receptor;

220) a membrane type metalloproteinase—nitrobenzylthioinosine-binding receptors or (nucleoside transporter);

221) a membrane type metalloproteinase—norepinephrine transporters;

222) a membrane type metalloproteinase—nucleoside transporter proteins;

223) a membrane type metalloproteinase—peripheral benzodiazepam binding receptors;

224) a membrane type metalloproteinase—seprase;

225) a membrane type metalloproteinase—sigma receptors;

226) a membrane type metalloproteinase—somatostatin receptors;

227) a membrane type metalloproteinase—stromelysin 3;

228) a membrane type metalloproteinase—trypsin;

229) a membrane type metalloproteinase—MMP 1;

230) a membrane type metalloproteinase—MMP 2;

231) a membrane type metalloproteinase—MMP 3;

232) a membrane type metalloproteinase—MMP 7;

233) a membrane type metalloproteinase—MMP 9;

234) a membrane type metalloproteinase—membrane type matrix metalloproteinase I;

235) a membrane type metalloproteinase—MMP 12;

236) a membrane type metalloproteinase—MMP 13;

237) a membrane type metalloproteinase—a tumor antigen;

238) alpha v beta 3 integrin—a cathepsin type protease;

239) alpha v beta 3 integrin—alpha v beta 3 integrin;

240) alpha v beta 3 integrin—bombesin/gastrin releasing peptide receptors;

241) alpha v beta 3 integrin—cathepsin B;

242) alpha v beta 3 integrin—cathepsin D;

243) alpha v beta 3 integrin—cathepsin K;

244) alpha v beta 3 integrin—cathepsin L;

245) alpha v beta 3 integrin—cathepsin 0;

246) alpha v beta 3 integrin—fibroblast activation protein;

247) alpha v beta 3 integrin—folate binding receptors;

248) alpha v beta 3 integrin—gastrin/cholecystokinin type B receptor;

249) alpha v beta 3 integrin—glutamate carboxypeptidase II or (PSMA);

250) alpha v beta 3 integrin—guanidinobenzoatase;

251) alpha v beta 3 integrin—laminin receptor;

252) alpha v beta 3 integrin—matrilysin;

253) alpha v beta 3 integrin—matripase;

254) alpha v beta 3 integrin—melanocyte stimulating hormone receptor;

255) alpha v beta 3 integrin—nitrobenzylthioinosine-binding receptors

or (nucleoside transporter);

256) alpha v beta 3 integrin—norepinephrine transporters;

257) alpha v beta 3 integrin—nucleoside transporter proteins;

258) alpha v beta 3 integrin—peripheral benzodiazepam binding

receptors;

259) alpha V beta 3 integrin—seprase;

260) alpha v beta 3 integrin—sigma receptors;

261) alpha v beta 3 integrin—somatostatin receptors;

262) alpha v beta 3 integrin—stromelysin 3;

263) alpha v beta 3 integrin—trypsin;

264) alpha v beta 3 integrin—MMP 1;

265) alpha v beta 3 integrin—MMP 2;

266) alpha v beta 3 integrin—MMP 3;

267) alpha v beta 3 integrin—MMP 7;

268) alpha v beta 3 integrin—MMP 9;

269) alpha v beta 3 integrin—membrane type matrix metalloproteinase I;

270) alpha v beta 3 integrin—MMP 12;

271) alpha v beta 3 integrin—MMP 13;

272) alpha v beta 3 integrin—a tumor antigen;

273) cathepsin B—a cathepsin type protease;

274) cathepsin B—bombesin/gastrin releasing peptide receptors;

275) cathepsin B—cathepsin B;

276) cathepsin B—cathepsin D;

277) cathepsin B—to cathepsin K;

278) cathepsin B—cathepsin L;

279) cathepsin B—cathepsin O;

280) cathepsin B—fibroblast activation protein;

281) cathepsin B—folate binding receptors;

282) cathepsin B—gastrin/cholecystokinin type B receptor;

283) cathepsin B—glutamate carboxypeptidase II or (PSMA);

284) cathepsin B—guanidinobenzoatase;

285) cathepsin B—laminin receptor;

286) cathepsin B—matrilysin;

287) cathepsin B—matripase;

288) cathepsin B—melanocyte stimulating hormone receptor;

289) cathepsin B—nitrobenzylthioinosine-binding receptors or (nucleoside transporter);

290) cathepsin B—norepinephrine transporters;

291) cathepsin B—nucleoside transporter proteins;

292) cathepsin B—peripheral benzodiazepam binding receptors;

293) cathepsin B—seprase;

294) cathepsin B—sigma receptors;

295) cathepsin B—somatostatin receptors;

296) cathepsin B—stromelysin 3;

297) cathepsin B—trypsin;

298) cathepsin B—MMP 1;

299) cathepsin B—MMP 2;

300) cathepsin B—MMP 3;

301) cathepsin B—MMP 7;

302) cathepsin B—MMP 9;

303) cathepsin B—membrane type matrix metalloproteinase I;

304) cathepsin B—MMP 12;

305) cathepsin B—MMP 13;

306) cathepsin B—a tumor antigen;

307) bombesin/gastrin releasing peptide receptors—a cathepsin type protease;

308) bombesin/gastrin releasing peptide receptors—bombesin/gastrin releasing peptide receptors;

309) bombesin/gastrin releasing peptide receptors—cathepsin B;

310) bombesin/gastrin releasing peptide receptors—cathepsin D;

311) bombesin/gastrin releasing peptide receptors—to cathepsin K;

312) bombesin/gastrin releasing peptide receptors—cathepsin L;

313) bombesin/gastrin releasing peptide receptors—cathepsin O;

314) bombesin/gastrin releasing peptide receptors—fibroblast activation protein;

315) bombesin/gastrin releasing peptide receptors—folate binding receptors;

316) bombesin/gastrin releasing peptide receptors—gastrin/cholecystokinin type B receptor;

317) bombesin/gastrin releasing peptide receptors—glutamate carboxypeptidase II or (PSMA);

318) bombesin/gastrin releasing peptide receptors—guanidinobenzoatase;

319) bombesin/gastrin releasing peptide receptors—laminin receptor;

320) bombesin/gastrin releasing peptide receptors—matrilysin;

321) bombesin/gastrin releasing peptide receptors—matripase;

322) bombesin/gastrin releasing peptide receptors—melanocyte stimulating hormone receptor;

323) bombesin/gastrin releasing peptide receptors —nitrobenzylthioinosine-binding receptors or (nucleoside transporter);

324) bombesin/gastrin releasing peptide receptors—norepinephrine transporters;

325) bombesin/gastrin releasing peptide receptors—nucleoside transporter proteins;

326) bombesin/gastrin releasing peptide receptors—peripheral benzodiazepam binding receptors;

327) bombesin/gastrin releasing peptide receptors—seprase;

328) bombesin/gastrin releasing peptide receptors—sigma receptors;

329) bombesin/gastrin releasing peptide receptors—somatostatin receptors;

330) bombesin/gastrin releasing peptide receptors—stromelysin 3;

331) bombesin/gastrin releasing peptide receptors—trypsin;

332) bombesin/gastrin releasing peptide receptors—MMP 1;

332) bombesin/gastrin releasing peptide receptors—MMP 2;

334) bombesin/gastrin releasing peptide receptors—MMP 3;

335) bombesin/gastrin releasing peptide receptors—MMP 7;

336) bombesin/gastrin releasing peptide receptors—MMP9;

337) bombesin/gastrin releasing peptide receptors—membrane type matrix metalloproteinase I;

338) bombesin/gastrin releasing peptide receptors—MMP 12;

339) bombesin/gastrin releasing peptide receptors—MMP 13;

340) bombesin/gastrin releasing peptide receptors—a tumor antigen;

341) fibroblast activation protein—a cathepsin type protease;

342) fibroblast activation protein—cathepsin D;

343) fibroblast activation protein—to cathepsin K;

344) fibroblast activation protein—cathepsin L;

345) fibroblast activation protein—cathepsin O;

346) fibroblast activation protein—fibroblast activation protein;

347) fibroblast activation protein—folate binding receptors;

348) fibroblast activation protein—gastrin/cholecystokinin type B

receptor;

349) fibroblast activation protein—glutamate carboxypeptidase II or (PSMA);

350) fibroblast activation protein—guanidinobenzoatase;

351) fibroblast activation protein—laminin receptor;

352) fibroblast activation protein—matrilysin;

353) fibroblast activation protein—matripase;

354) fibroblast activation protein—melanocyte stimulating hormone receptor;

355) fibroblast activation protein—nitrobenzylthioinosine-binding receptors or (nucleoside transporter);

356) fibroblast activation protein—norepinephrine transporters;

357) fibroblast activation protein—nucleoside transporter proteins;

358) fibroblast activation protein—peripheral benzodiazepam binding receptors;

359) fibroblast activation protein—plasmin;

360) fibroblast activation protein—seprase;

361) fibroblast activation protein—sigma receptors;

362) fibroblast activation protein—somatostatin receptors;

363) fibroblast activation protein—stromelysin 3;

364) fibroblast activation protein—trypsin;

365) fibroblast activation protein—MMP 1;

366) fibroblast activation protein—MMP 2;

367) fibroblast activation protein—MMP 3;

368) fibroblast activation protein—MMP 7;

369) fibroblast activation protein—MMP 9;

370) fibroblast activation protein—membrane type matrix metalloproteinase I;

371) fibroblast activation protein—MMP 12;

372) fibroblast activation protein—MMP 13;

373) fibroblast activation protein—a tumor antigen;

374) glutamate carboxypeptidase II or PSMA—cathepsin D;

375) glutamate carboxypeptidase II or PSMA—to cathepsin K;

376) glutamate carboxypeptidase II or PSMA—cathepsin L;

377) glutamate carboxypeptidase II or PSMA—cathepsin O;

378) glutamate carboxypeptidase II or PSMA—fibroblast activation protein;

379) glutamate carboxypeptidase II or PSMA—folate binding receptors;

380) glutamate carboxypeptidase II or PSMA—gastrin/cholecystokinin type B receptor;

381) glutamate carboxypeptidase II or PSMA—glutamate

carboxypeptidase II or (PSMA);

382) glutamate carboxypeptidase II or PSMA—guanidinobenzoatase;

383) glutamate carboxypeptidase II or PSMA—laminin receptor;

384) glutamate carboxypeptidase II or PSMA—matrilysin;

385) glutamate carboxypeptidase II or PSMA—matripase;

386) glutamate carboxypeptidase II or PSMA—melanocyte stimulating hormone receptor;

387) glutamate carboxypeptidase II or PSMA—nitrobenzylthioinosine-binding receptors or (nucleoside transporter);

388) glutamate carboxypeptidase II or PSMA—nucleoside transporter proteins;

389) glutamate carboxypeptidase II or PSMA—peripheral benzodiazepam binding receptors;

390) glutamate carboxypeptidase II or PSMA—seprase;

391) glutamate carboxypeptidase II or PSMA—sigma receptors;

392) glutamate carboxypeptidase II or PSMA—somatostatin receptors;

393) glutamate carboxypeptidase II or PSMA—stromelysin 3;

394) glutamate carboxypeptidase II or PSMA—trypsin;

395) glutamate carboxypeptidase II or PSMA—MMP 1;

396) glutamate carboxypeptidase II or PSMA—MMP 2;

397) glutamate carboxypeptidase II or PSMA—MMP 3;

398) glutamate carboxypeptidase II or PSMA—MMP 7;

399) glutamate carboxypeptidase II or PSMA—MMP 9;

400) glutamate carboxypeptidase II or PSMA—membrane type matrix metalloproteinase I;

401) glutamate carboxypeptidase II or PSMA—MMP 12;

402) glutamate carboxypeptidase II or PSMA—MMP 13;

403) glutamate carboxypeptidase II or PSMA—a tumor antigen;

404) laminin receptor—a cathepsin type protease;

405) laminin receptor—cathepsin B;

406) laminin receptor—cathepsin D;

407) laminin receptor—to cathepsin K;

408) laminin receptor—cathepsin L;

409) laminin receptor—cathepsin O;

410) laminin receptor—fibroblast activation protein;

411) laminin receptor—folate binding receptors;

412) laminin receptor—gastrin/cholecystokinin type B receptor;

413) laminin receptor—guanidinobenzoatase;

414) laminin receptor—laminin receptor;

415) laminin receptor—matrilysin;

416) laminin receptor—matripase;

417) laminin receptor—melanocyte stimulating hormone receptor;

418) laminin receptor—nitrobenzylthioinosine-binding receptors or (nucleoside transporter);

419) laminin receptor—norepinephrine transporters;

420) laminin receptor—nucleoside transporter proteins;

421) laminin receptor—peripheral benzodiazepam binding receptors;

422) laminin receptor—seprase;

423) laminin receptor—sigma receptors;

424) laminin receptor—somatostatin receptors;

425) laminin receptor—stromelysin 3;

426) laminin receptor—trypsin;

427) laminin receptor—MMP 1;

428) laminin receptor—MMP 2;

429) laminin receptor—MMP 3;

430) laminin receptor—MMP 7;

431) laminin receptor—MMP 9;

432) laminin receptor—membrane type matrix metalloproteinase I;

433) laminin receptor—MMP 12;

434) laminin receptor—MMP 13;

435) laminin receptor—a tumor antigen;

436) seprase—a cathepsin type protease;

437) seprase—cathepsin D;

438) seprase—to cathepsin K;

439) seprase—cathepsin L;

440) seprase—cathepsin O;

441) seprase—fibroblast activation protein;

442) seprase—folate binding receptors;

443) seprase—gastrin/cholecystokinin type B receptor;

444) seprase—guanidinobenzoatase;

445) seprase—matripase;

446) seprase—melanocyte stimulating hormone receptor;

447) seprase—nitrobenzylthioinosine-binding receptors or (nucleoside transporter);

448) seprase—norepinephrine transporters;

449) seprase—nucleoside transporter proteins;

450) seprase—peripheral benzodiazepam binding receptors;

451) seprase—seprase;

452) seprase—sigma receptors;

453) seprase—somatostatin receptors;

454) seprase—stromelysin 3;

455) seprase—trypsin;

456) seprase—MMP 1;

457) seprase—MMP 2;

458) seprase—MMP 3;

459) seprase—MMP 7;

460) seprase—MMP 9;

461) seprase—membrane type matrix metalloproteinase I;

462) seprase—MMP 12;

463) seprase—MMP 13;

464) seprase—a tumor antigen;

465) guanidinobenzoatase—a cathepsin type protease;

466) guanidinobenzoatase—cathepsin D;

467) guanidinobenzoatase—to cathepsin K;

468) guanidinobenzoatase—cathepsin L;

469) guanidinobenzoatase—cathepsin O;

470) guanidinobenzoatase—fibroblast activation protein;

471) guanidinobenzoatase—folate binding receptors;

472) guanidinobenzoatase—gastrin/cholecystokinin type B receptor;

473) guanidinobenzoatase—guanidinobenzoatase;

474) guanidinobenzoatase—matripase;

475) guanidinobenzoatase—melanocyte stimulating hormone

receptor;

476) guanidinobenzoatase—nitrobenzylthioinosine-binding receptors or (nucleoside transporter);

477) guanidinobenzoatase—norepinephrine transporters;

478) guanidinobenzoatase—nucleoside transporter proteins;

479) guanidinobenzoatase—peripheral benzodiazepam binding receptors;

480) guanidinobenzoatase—sigma receptors;

481) guanidinobenzoatase—somatostatin receptors;

482) guanidinobenzoatase—stromelysin 3;

483) guanidinobenzoatase—trypsin;

484) guanidinobenzoatase—MMP 1;

485) guanidinobenzoatase—MMP 2;

486) guanidinobenzoatase—MMP 3;

487) guanidinobenzoatase—MMP 7;

488) guanidinobenzoatase—MMP 9;

489) guanidinobenzoatase—membrane type matrix metalloproteinase I;

490 ) guanidinobenzoatase—MMP 12;

491) guanidinobenzoatase—MMP 13;

492) guanidinobenzoatase—a tumor antigen;

493) peripheral benzodiazepam binding receptors—a cathepsin type protease;

494) peripheral benzodiazepam binding receptors—cathepsin D;

495) peripheral benzodiazepam binding receptors—to cathepsin K;

496) peripheral benzodiazepam binding receptors—cathepsin L;

497) peripheral benzodiazepam binding receptors—cathepsin O;

498) peripheral benzodiazepam binding receptors—fibroblast activation protein;

499) peripheral benzodiazepam binding receptors—folate binding receptors;

500) peripheral benzodiazepam binding receptors

gastrin/cholecystokinin type B receptor;

501) peripheral benzodiazepam binding receptors—guanidinobenzoatase;

502) peripheral benzodiazepam binding receptors matripase;

503) peripheral benzodiazepam binding receptors—melanocyte stimulating hormone receptor;

504) peripheral benzodiazepam binding receptors nitrobenzylthioinosine-binding receptors or (nucleoside transporter);

505) peripheral benzodiazepam binding receptors—norepinephrine transporters;

506) peripheral benzodiazepam binding receptors—nucleoside transporter proteins;

507) peripheral benzodiazepam binding receptors—peripheral benzodiazepam binding receptors;

508) peripheral benzodiazepam binding receptors—sigma receptors;

509) peripheral benzodiazepam binding receptors—somatostatin receptors;

510) peripheral benzodiazepam binding receptors—stromelysin 3;

511) peripheral benzodiazepam binding receptors—trypsin;

512) peripheral benzodiazepam binding receptors—MMP 1;

513) peripheral benzodiazepam binding receptors—MMP 2;

514) peripheral benzodiazepam binding receptors—MMP 3;

515) peripheral benzodiazepam binding receptors—MMP 7;

516) peripheral benzodiazepam binding receptors—MMP 9;

517) peripheral benzodiazepam binding receptors—membrane type matrix metalloproteinase I;

518) peripheral benzodiazepam binding receptors—MMP 12;

519) peripheral benzodiazepam binding receptors—MMP 13;

520) peripheral benzodiazepam binding receptors—a tumor antigen;

521) folate binding receptors—a cathepsin type protease;

522) folate binding receptors—cathepsin D;

523) folate binding receptors—to cathepsin K;

524) folate binding receptors—cathepsin L;

525) folate binding receptors—cathepsin O;

526) folate binding receptors—fibroblast activation protein;

527) folate binding receptors—folate binding receptors;

528) folate binding receptors—matripase;

529) folate binding receptors—melanocyte stimulating hormone receptor;

530) folate binding receptors—nitrobenzylthioinosine-binding receptors or (nucleoside transporter);

531) folate binding receptors—norepinephrine transporters;

532) folate binding receptors—nucleoside transporter proteins;

533) folate binding receptors—sigma receptors;

534) folate binding receptors—somatostatin receptors;

535) folate binding receptors—stromelysin 3;

536) folate binding receptors—trypsin;

537) folate binding receptors—MMP;

538) folate binding receptors—MMP 2;

539) folate binding receptors—MMP 3;

540) folate binding receptors—MMP 7;

541) folate binding receptors—MMP 9;

542) folate binding receptors—membrane type matrix metalloproteinase I;

543) folate binding receptors—MMP 12;

544) folate binding receptors—MMP 13;

545) folate binding receptors—a tumor antigen;

546) folate binding receptors—a cathepsin type protease;

547) folate binding receptors—cathepsin D;

548) folate binding receptors—to cathepsin K;

549) folate binding receptors—cathepsin L;

550) folate binding receptors—cathepsin O;

551) folate binding receptors—fibroblast activation protein;

552) folate binding receptors—folate binding receptors;

553) folate binding receptors—matripase;

554) folate binding receptors—melanocyte stimulating hormone receptor;

555) folate binding receptors—nitrobenzylthioinosine-binding receptors or (nucleoside transporter);

556) folate binding receptors—norepinephrine transporters;

557) folate binding receptors—nucleoside transporter proteins;

558) folate binding receptors—sigma receptors;

559) folate binding receptors—somatostatin receptors;

560) folate binding receptors—stromelysin 3;

561) folate binding receptors—trypsin;

562) folate binding receptors—MMP 1;

563) folate binding receptors—MMP 2;

564) folate binding receptors—MMP 3;

565) folate binding receptors—MMP 7;

566) folate binding receptors—MMP 9;

567) folate binding receptors—membrane type matrix metalloproteinase I;

568) folate binding receptors—MMP 12;

569) folate binding receptors—MMP 13;

570) folate binding receptors—a tumor antigen;

571) nucleoside transporter proteins—a cathepsin type protease;

572) nucleoside transporter proteins—cathepsin D;

573) nucleoside transporter proteins—to cathepsin K;

574) nucleoside transporter proteins—cathepsin L;

575) nucleoside transporter proteins—cathepsin O;

576) nucleoside transporter proteins—fibroblast activation protein;

577) nucleoside transporter proteins—nucleoside transporter proteins;

578) nucleoside transporter proteins—matripase;

579) nucleoside transporter proteins—melanocyte stimulating hormone receptor;

580) nucleoside transporter proteins—nitrobenzylthioinosine-binding receptors or (nucleoside transporter);

581) nucleoside transporter proteins—norepinephrine transporters;

582) nucleoside transporter proteins—nucleoside transporter proteins;

583) nucleoside transporter proteins—sigm a receptors;

584) nucleoside transporter proteins—somatostatin receptors;

585) nucleoside transporter proteins—stromelysin 3;

586) nucleoside transporter proteins—trypsin;

587) nucleoside transporter proteins—MMP 1;

588) nucleoside transporter proteins—MMP 2;

589) nucleoside transporter proteins—MMP 3;

590) nucleoside transporter proteins—MMP 7;

591) nucleoside transporter proteins—MMP 9;

592) nucleoside transporter proteins—membrane type matrix metalloproteinase I;

593) nucleoside transporter proteins—MMP 12;

594) nucleoside transporter proteins—MMP 13;

595) nucleoside transporter proteins—a tumor antigen;

596) melanocyte stimulating hormone receptor—a cathepsin type protease;

597) melanocyte stimulating hormone receptor—cathepsin D;

598) melanocyte stimulating hormone receptor—to cathepsin K;

599) melanocyte stimulating hormone receptor—cathepsin L;

600) melanocyte stimulating hormone receptor—cathepsin O;

601) melanocyte stimulating hormone receptor—fibroblast activation protein;

602) melanocyte stimulating hormone receptor—melanocyte stimulating hormone receptor;

603) melanocyte stimulating hormone receptor—melanocyte stimulating hormore receptor;

604) melanocyte stimulating hormone receptor—nitrobenzylthioinosine-binding receptors or (nucleoside transporter);

605) melanocyte stimulating hormone receptor—norepinephrine transporters;

606) melanocyte stimulating hormone receptor—nucleoside transporter proteins;

607) melanocyte stimulating hormone receptor—sigma receptors;

608) melanocyte stimulating hormone receptor—somatostatin receptors;

609) melanocyte stimulating hormone receptor—stromelysin 3;

610) melanocyte stimulating hormone receptor—trypsin;

611) melanocyte stimulating hormone receptor—MMP 1;

612) melanocyte stimulating hormone receptor—MMP 2;

613) melanocyte stimulating hormone receptor—MMP 3;

614) melanocyte stimulating hormone receptor—MMP 7;

615) melanocyte stimulating hormone receptor—MMP 9;

616) melanocyte stimulating hormone receptor—membrane type matrix metalloproteinase I;

617) melanocyte stimulating hormone receptor—MMP 12;

618) melanocyte stimulating hormone receptor—MMP 13;

619) melanocyte stimulating hormone receptor—a tumor antigen;

620) sigma receptors—a cathepsin type protease;

621) sigma receptors—cathepsin D;

622) sigma receptors—to cathepsin K;

623) sigma receptors—cathepsin L;

624) sigma receptors—cathepsin O;

625) sigma receptors—fibroblast activation protein;

626) sigma receptors—sigma receptors;

627) sigma receptors—matripase;

628) sigma receptors—norepinephrine transporters;

629) sigma receptors—sigma receptors;

630) sigma receptors—somatostatin receptors;

631) sigma receptors—stromelysin 3;

632) sigma receptors—trypsin;

633) sigma receptors—MMP 1;

634) sigma receptors—MMP 2;

635) sigma receptors—MMP 3;

636) sigma receptors—MMP 7;

637) sigma receptors—MMP 9;

638) sigma receptors—membrane type matrix metalloproteinase I;

639) sigma receptors—MMP 12;

640) sigma receptors—MMP 13;

641) sigma receptors—a tumor antigen

642) somatostatin receptors—a cathepsin type protease;

643) somatostatin receptors—cathepsin D;

644) somatostatin receptors—to cathepsin K;

645) somatostatin receptors—cathepsin L;

646) somatostatin receptors—cathepsin O;

647) somatostatin receptors—fibroblast activation protein;

648) somatostatin receptors—somatostatin receptors;

649) somatostatin receptors—matripase;

650) somatostatin receptors—melanocyte stimulating hormone receptor;

651) somatostatin receptors—sigma receptors;

652) somatostatin receptors—somatostatin receptors;

653) somatostatin receptors—stromelysin 3;

654) somatostatin receptors—trypsin;

655) somatostatin receptors—MMP 1;

656) somatostatin receptors—MMP 2;

657) somatostatin receptors—MMP 3;

658) somatostatin receptors—MMP 7;

659) somatostatin receptors—MMP 9;

660) somatostatin receptors—membrane type matrix metalloproteinase I;

661) somatostatin receptors—MMP 12;

662) somatostatin receptors—MMP 13;

663) somatostatin receptors—a tumor antigen;

664) stromelysin 3—a cathepsin type protease;

665) stromelysin 3—cathepsin D;

666) stromelysin 3—to cathepsin K;

667) stromelysin 3—cathepsin L;

668) stromelysin 3—cathepsin O;

669) stromelysin 3—fibroblast activation protein;

670) stromelysin 3—stromelysin 3;

671) stromelysin 3—matripase;

672) stromelysin 3—melanocyte stimulating hormone receptor;

673) stromelysin 3—somatostatin receptors;

674) stromelysin 3—trypsin;

675) stromelysin 3—MMP 1;

676) stromelysin 3—MMP 2;

677) stromelysin 3—MMP 3;

678) stromelysin 3—MMP 7;

679) stromelysin 3—MMP 9;

680) stromelysin 3—membrane type matrix metalloproteinase I;

681) stromelysin 3—MMP 12;

682) stromelysin 3—MMP 13;

683) stromelysin 3—a tumor antigen;

684) trypsin—a cathepsin type protease;

685) trypsin—cathepsin D;

686) trypsin—to cathepsin K;

687) trypsin—cathepsin L;

688) trypsin—cathepsin O;

689) trypsin—fibroblast activation protein;

690) trypsin—trypsin;

691) trypsin—matripase;

692) trypsin—melanocyte stimulating hormone receptor;

693) trypsin—stromelysin 3;

694) trypsin—MMP 1;

695) trypsin—MMP 2;

696) trypsin—MMP 3;

697) trypsin—MMP 7;

698) trypsin—MMP 9;

699) trypsin—membrane type matrix metalloproteinase I;

700) trypsin—MMP 12;

701) trypsin—MMP 13;

702) trypsin—a tumor antigen;

703) MMP 1—a cathepsin type protease;

704) MMP 1—cathepsin D;

705) MMP 1—to cathepsin K;

706) MMP 1—cathepsin L;

707) MMP 1—cathepsin O;

708) MMP 1—fibroblast activation protein;

709) MMP 1—matripase;

710) MMP 1—melanocyte stimulating hormone receptor;

711) MMP 1—stromelysin 3;

712) MMP 1—MMP 1;

713) MMP 1—MMP 2;

714) MMP 1—MMP 3;

715) MMP 1—MMP 7;

716) MMP 1—MMP9;

717) MMP 1—membrane type matrix metalloproteinase 1;

718) MMP 1—MMP 12;

719) MMP 1—MMP 13;

720) MMP 1—a tumor antigen;

721) MMP-2—a cathepsin type protease;

722) MMP-2—cathepsin D;

723) MMP-2—to cathepsin K;

724) MMP-2—cathepsin L;

725) MMP-2—cathepsin O;

726) MMP-2—fibroblast activation protein;

727) MMP-2—matripase;

728) MMP-2—melanocyte stimulating hormone receptor;

729) MMP-2—stromelysin 3;

730) MMP-2—MMP 2;

731) MMP-2—MMP 3;

732) MMP-2—MMP7;

733) MMP-2—MMP 9;

734) MMP-2—membrane type matrix metalloproteinase I;

735) MMP-2—MMP-2;

736) MMP-2—MMP-3;

737) MMP-2—a tumor antigen;

738) MMP-3—a cathepsin type protease;

739) MMP-3—cathepsin D;

740) MMP-3—to cathepsin K;

741) MMP-3—cathepsin L;

742) MMP-3—cathepsin O;

743) MMP-3—matripase;

744) MMP-3—MMP 3;

745) MMP-3—MMP 7;

746) MMP-3—MMP 9;

747) MMP-3—membrane type matrix metalloproteinase I;

748) MMP-3—MMP-3;

749) MMP-3—a tumor antigen;

750) MMP 7—a cathepsin type protease;

751) MMP 7—cathepsin D;

752) MMP 7—to cathepsin K;

753) MMP 7—cathepsin L;

754) MMP 7—cathepsin O;

755) MMP 7—fibroblast activation protein;

756) MMP 7—matripase;

757) MMP 7—stromelysin 3;

758) MMP 7—MMP 7;

759) MMP 7—MMP 9;

760) MMP 7—membrane type matrix metalloproteinase I;

761) MMP 7—a tumor antigen;

762) MMP 9—a cathepsin type protease;

763) MMP 9—cathepsin D;

764) MMP 9—to cathepsin K;

765) MMP 9—cathepsin L;

766) MMP 9—cathepsin O;

767) MMP 9—matripase;

768) MMP 9—MMP 9;

769) MMP 9—membrane type matrix metalloproteinase I;

770) MMP 9—a tumor antigen;

771) MMP 12—a cathepsin type protease;

772) MMP 12—cathepsin D;

773) MMP 12—to cathepsin K;

774) MMP 12—cathepsin L;

775) MMP 12—cathepsin O;

776) MMP 12—matripase;

777) MMP 12—MMP 2;

778) MMP 12—membrane type matrix metalloproteinase I;

779) MMP 12—a tumor antigen;

780) MMP 13—a cathepsin type protease;

781) MMP 13—cathepsin D;

782) MMP 13—to cathepsin K;

783) MMP 13—cathepsin L;

784) MMP 13—cathepsin O;

785) MMP 13—matripase;

786) MMP 13—membrane type matrix metalloproteinase I;

787) MMP 13—a tumor antigen;

788) Membrane type matrix metalloproteinase—a cathepsin type protease;

789) Membrane type matrix metalloproteinase—cathepsin D;

790) Membrane type matrix metalloproteinase—to cathepsin K;

791) Membrane type matrix metalloproteinase—cathepsin L;

792) Membrane type matrix metalloproteinase—cathepsin O;

793) Membrane type matrix metalloproteinase—matripase;

794) Membrane type matrix metalloproteinase—membrane type matrix metalloproteinase I;

795) and Membrane type matrix metalloproteinase—a tumor antigen.

In preferred embodiments of (embodiments TLP #.X, wherein X=1, 2, 3, . . . 795), the structure of the respective targeting ligands are of embodiments TL#Z (wherein Z=1, 2, 3 . . . 44) or as described in the targeting ligand neoantigen sections of this document.

The scope of the present invention includes a compound comprised of one of the pairs of tumor targeting ligands listed above and an effector agent with anti-cancer activity.

In a preferred embodiment, ET is comprised of a third targeting receptor that is enriched on a tumor cell and a pair of targeting receptors selected from the list above. In a preferred embodiment this third targeting receptor binds to PSMA.

In preferred embodiments (designated 0.neoA) the compound ET is an anti-cancer drug comprised of at least one targeting ligand that is increased on a tumor cell compared to a normal cell and an effector agent that can irreversibly chemically modify a component of tumors that is also increased at a tumor cell compared to a normal cell. In a preferred embodiment (desiginated 1.neoA) the number of targeting ligands is one. In a preferred embodiment (designated 2.neoA) the number of targeting ligands is two. In a preferred embodiment (designated 3.neoA) the number of targeting ligands is three. These compounds are useful in the method of target neaoantigen immunotherapy described in a latter section of this document.

In a preferred embodiment of the above embodiments, (0.neoA and 1neoA and 2.neoA, and 3.neoA); the tumor component that can be irreversibly modified is Prostate Specific Antigen, or Human glandular kallikrein 2, or Prostatic acid phosphatase, or Plasmin, or Placental type alkaline phosphatase, or Matriptase, or A Matrix metalloproteinases, or Thymidine phosphorylase, or Trypsin, or Urokinase, or Fatty Acid Synthase, or Steroid sulfatase, or Epidermal growth factor receptors, or Mitogen activated protein kinase kinase, or Phosphatidylinositol 3-kinase, or Mitogen activated protein kinase, or an Estrogen receptor, or Thymidylate synthase, or Protein kinase A, or Fibroblast activation protein or seprase, or P-glycoprotein, or Ribonucleotide diphosphate reductase, or Dihydrofolate reductase, or Src Kinases, or Platelet-derived growth factor receptors, or MMP 7, or MMP 1, or MMP 2, or MMP 3, or MMP 9, or MMP 12, or MMP 13, or Membrane type MMP 1, or A Cathepsin, or Cathepsin B, or Glutathione S -Transferases.

In a preferred embodiment, of (embodiment TLP #.X, for X=1, 2, 3, . . . 795) the effector agent is comprised of a group that can irreversibly chemically modify Prostate Specific Antigen.

In a preferred embodiment, of (embodiment TLP #.X, for X=1, 2, 3, . . . 795) the effector agent is comprised of a group that can irreversibly chemically modify Human glandular kallikrein 2.

In a preferred embodiment, of (embodiment TLP #.X, for X=1, 2, 3, . . . 795) the effector agent is comprised of a group that can irreversibly chemically modify Prostatic acid phosphatase.

In a preferred embodiment, of (embodiment TLP #.X, for X=1, 2, 3, . . . 795) the effector agent is comprised of a group that can irreversibly chemically modify Plasmin.

In a preferred embodiment, of (embodiment TLP #.X, for X=1, 2, 3, . . . 795) the effector agent is comprised of a group that can irreversibly chemically modify Placental type alkaline phosphatase.

In a preferred embodiment, of (embodiment TLP #.X, for X=1, 2, 3, . . . 795) the effector agent is comprised of a group that can irreversibly chemically modify Matriptase.

In a preferred embodiment, of (embodiment TLP #.X, for X=1, 2, 3, . . . 795) the effector agent is comprised of a group that can irreversibly chemically modify A Matrix metalloproteinases.

In a preferred embodiment, of (embodiment TLP #.X, for X=1, 2, 3, . . . 795) the effector agent is comprised of a group that can irreversibly chemically modify Thymidine phosphorylase.

In a preferred embodiment, of (embodiment TLP #.X, for X=1, 2, 3, . . . 795) the effector agent is comprised of a group that can irreversibly chemically modify Trypsin.

In a preferred embodiment, of (embodiment TLP #.X, for X=1, 2, 3, . . . 795) the effector agent is comprised of a group that can irreversibly chemically modify Urokinase.

In a preferred embodiment, of (embodiment TLP #.X, for X=1, 2, 3, . . . 795) the effector agent is comprised of a group that can irreversibly chemically modify Fatty Acid Synthase.

In a preferred embodiment, of (embodiment TLP #.X, for X=1, 2, 3, . . . 795) the effector agent is comprised of a group that can irreversibly chemically modify Steroid sulfatase.

In a preferred embodiment, (embodiment TLP #.X, for X=1, 2, 3, . . . 795) the effector agent is comprised of a group that can irreversibly chemically modify Epidermal growth factor receptors.

In a preferred embodiment, of (embodiment TLP #.X, for X=1, 2, 3, . . . 795) the effector agent is comprised of a group that can irreversibly chemically modify Mitogen activated protein kinase kinase.

In a preferred embodiment, of (embodiment TLP #.X, for X=1, 2, 3, . . . 795) the effector agent is comprised of a group that can irreversibly chemically modify Phosphatidylinositol.

In a preferred embodiment, of (embodiment TLP #.X, for X=1, 2, 3, . . . 795) the effector agent is comprised of a group that can irreversibly chemically modify 3-kinase.

In a preferred embodiment, of (embodiment TLP #.X, for X=1, 2, 3, . . . 795) the effector agent is comprised of a group that can irreversibly chemically modify Mitogen activated protein kinase.

In a preferred embodiment, of (embodiment TLP #.X, for X=1, 2, 3, . . . 795) the effector agent is comprised of a group that can irreversibly chemically modify an Estrogen receptor.

In a preferred embodiment, of (embodiment TLP #.X, for X=1, 2, 3, . . . 795) the effector agent is comprised of a group that can irreversibly chemically modify Thymidylate synthase.

In a preferred embodiment, of (embodiment TLP #.X, for X=1, 2, 3, . . . 795) the effector agent is comprised of a group that can irreversibly chemically modify Protein kinase A.

In a preferred embodiment, of (embodiment TLP #.X, for X=1, 2, 3, . . . 795) the effector agent is comprised of a group that can irreversibly chemically modify Fibroblast activation protein or seprase.

In a preferred embodiment, of (embodiment TLP #.X, for X=1, 2, 3, . . . 795) the effector agent is comprised of a group that can irreversibly chemically modify P-glycoprotein.

In a preferred embodiment, of (embodiment TLP #.X, for X=1, 2, 3, . . . 795) the effector agent is comprised of a group that can irreversibly chemically modify Ribonucleotide diphosphate reductase.

In a preferred embodiment, of (embodiment TLP #.X, for X=1, 2, 3, . . . 795) the effector agent is comprised of a group that can irreversibly chemically modify Dihydrofolate reductase.

In a preferred embodiment, of (embodiment TLP #.X, for X=1, 2, 3, . . . 795) the effector agent is comprised of a group that can irreversibly chemically modify Src Kinases.

In a preferred embodiment, of (embodiment TLP #.X, for X=1, 2, 3, . . . 795) the effector agent is comprised of a group that can irreversibly chemically modify Platelet-derived growth factor receptors.

In a preferred embodiment, of (embodiment TLP #.X, for X=1, 2, 3, . . . 795) the effector agent is comprised of a group that can irreversibly chemically modify MMP 7.

In a preferred embodiment, of (embodiment TLP #.X, for X=1, 2, 3, . . . 795) the effector agent is comprised of a group that can irreversibly chemically modify MMP 1.

In a preferred embodiment, of (embodiment TLP #.X, for X=1, 2, 3, . . . 795) the effector agent is comprised of a group that can irreversibly chemically modify MMP 2.

In a preferred embodiment, of (embodiment TLP #.X, for X=1, 2, 3, . . . 795) the effector agent is comprised of a group that can irreversibly chemically modify MMP 3.

In a preferred embodiment, of (embodiment TLP #.X, for X=1, 2, 3, . . . .795) the effector agent is comprised of a group that can irreversibly chemically modify MMP 9.

In a preferred embodiment, of (embodiment TLP #.X, for X=1, 2, 3, . . . 795) the effector agent is comprised of a group that can irreversibly chemically modify MMP 12.

In a preferred embodiment, of (embodiment TLP #.X, for X=1, 2, 3, . . . 795) the effector agent is comprised of a group that can irreversibly chemically modify MMP 13.

In a preferred embodiment, of (embodiment TLP #.X, for X=1, 2, 3, . . . 795) the effector agent is comprised of a group that can irreversibly chemically modify Membrane type MMP 1.

In a preferred embodiment, of (embodiment TLP #.X, for X=1, 2, 3, . . . 795) the effector agent is comprised of a group that can irreversibly chemically modify a cathepsin.

In a preferred embodiment, of (embodiment TLP #.X, for X=1, 2, 3, . . . 795) the effector agent is comprised of a group that can irreversibly chemically modify Cathepsin B.

In a preferred embodiment, of (embodiment TLP #.X, for X=1, 2, 3, . . . 795) the effector agent is comprised of a group that can irreversibly chemically modify Glutathione S -Transferases.

In a preferred embodiment ET is an anti-cancer drug comprised of a pair of targeting ligands that bind to a pair of targeting receptors (a1 - - - a2) listed above or a pair of said targeting ligands and a third tumor-selective targeting ligand; and wherein the effector agents are comprised of one or more cytotoxic agents selected from the following list:

1. anthracyclines

2. ellipticines

3. mitoxantrones

4. bleomycins

5. taxols

6. inhibitors of thymidylate synthase

7. hydroxystaurosporine

8. cryptophycin analogs

9. vincristine

10. vinblastine

11. indanocine

12. mitomycin c

13. phosphoramide mustard analogs

14. podophyllotoxins

15. ecteinascidins

16. didemnin

17. BW1843U89

18. 2-pyrrolinodoxorubicin

19. phthalascidin

20. an inhibitor of glycinamide ribonucleotide transformylase

21. an inhibitor hypoxanthene-guanine phosphoribosyltransferase

22. campothecin

23. trimetrexate

24. a nucleoside transporter inhibitor

25. mycophenolic acid

26. an inhibitor of dihydroorotic acid dehydrogenase

27. an inhibitor to Orotidine 5′-phosphate decarboxylase

28. a radionuclide

In a preferred embodiment, of the above the embodiment, the number of anti-cancer drugs from the list that comprises E is 1, or 2.

In a preferred embodiment, of (embodiment TLP #.X, for X=1, 2, 3, . . . 795) the effector agent is comprised of anthracyclines.

In a preferred embodiment, of (embodiment TLP #.X, for X=1, 2, 3, . . . 795) the effector agent is comprised of ellipticines.

In a preferred embodiment, of (embodiment TLP #.X, for X=1, 2, 3, . . . 795) the effector agent is comprised of mitoxantrones.

In a preferred embodiment, of (embodiment TLP #.X, for X=1, 2, 3, . . . 795) the effector agent is comprised of Bleomycin.

In a preferred embodiment, of (embodiment TLP #.X, for X=1, 2, 3, . . . 795) the effector agent is comprised of taxol.

In a preferred embodiment, of (embodiment TLP #.X, for X=1, 2, 3, . . . 795) the effector agent is comprised of an inhibitor of thymidylate synthase.

In a preferred embodiment, of (embodiment TLP #.X, for X=1, 2, 3, . . . 795) the effector agent is comprised of Hydroxystaurosporine.

In a preferred embodiment, of (embodiment TLP #.X, for X=1, 2, 3, . . . 795) the effector agent is comprised of a cryptophycin analogs.

In a preferred embodiment, of (embodiment TLP #.X, for X=1, 2, 3, . . . 795) the effector agent is comprised of Vincristine.

In a preferred embodiment, of (embodiment TLP #.X, for X=1, 2, 3, . . . 795) the effector agent is comprised of Vinblastine.

In a preferred embodiment, of (embodiment TLP #.X, for X=1, 2, 3, . . . 795) the effector agent is comprised of Indanocine.

In a preferred embodiment, of (embodiment TLP #.X, for X=1, 2, 3, . . . 795) the effector agent is comprised of mitomycin c.

In a preferred embodiment, of (embodiment TLP #.X, for X=1, 2, 3, . . . 795) the effector agent is comprised of a phosphoramide mustard analogs.

In a preferred embodiment, of (embodiment TLP #.X, for X=1, 2, 3, . . . 795) the effector agent is comprised of Podophyllotoxins.

In a preferred embodiment, of (embodiment TLP #.X, for X=1, 2, 3, . . . 795) the effector agent is comprised of Ecteinascidins.

In a preferred embodiment, of (embodiment TLP #.X, for X=1, 2, 3, . . . 795) the effector agent is comprised of a Didemnin.

In a preferred embodiment, of (embodiment TLP #.X, for X=1, 2, 3, . . . 795) the effector agent is comprised of BW1843U89.

In a preferred embodiment, of (embodiment TLP #.X, for X=1, 2, 3, . . . 795) the effector agent is comprised of 2-pyrrolinodoxorubicin.

In a preferred embodiment, of (embodiment TLP #.X, for X=1, 2, 3, . . . 795) the effector agent is comprised of a Phthalascidin.

In a preferred embodiment, of (embodiment TLP #.X, for X=1, 2, 3, . . . 795) the effector agent is comprised of an inhibitor of glycinamide ribonucleotide transformylase.

In a preferred embodiment, of (embodiment TLP #.X, for X=1, 2, 3, . . . 795) the effector agent is comprised of an inhibitor hypoxanthene-guanine phosphoribosyltransferase.

In a preferred embodiment, of (embodiment TLP #.X, for X=1, 2, 3, . . . 795) the effector agent is comprised of Campothecin.

In a preferred embodiment, of (embodiment TLP #.X, for X=1, 2, 3, . . . 795) the effector agent is comprised of Trimetrexate.

In a preferred embodiment, of (embodiment TLP #.X, for X=1, 2, 3, . . . 795) the effector agent is comprised of a nucleoside transporter inhibitor.

In a preferred embodiment, of (embodiment TLP #.X, for X=1, 2, 3, . . . 795) the effector agent is comprised of mycophenolic acid.

In a preferred embodiment, (embodiment TLP #.X, for X=1, 2, 3, . . . 795) the effector agent is comprised of an inhibitor of dihydroorotic acid dehydrogenase.

In a preferred embodiment, of (embodiment TLP #.X, for X=1, 2, 3, . . . 795) the effector agent is comprised of an inhibitor to Orotidine 5′-phosphate decarboxylase.

In a preferred embodiment, of (embodiment TLP #.X, for X=1, 2, 3, . . . 795) the effector agent is comprised of a radionuclide.

In Preferred Embodiments;

(referred to as embodiments “OSTLP #X”, for X=1, 2, 3, 4 . . . 40 wherein X is the number of the targeting receptor in the list below);

E1T1 and E2T2 are a set of anti-cancer drugs for use together, wherein E1 and E2 are effector agents that exhibit synergistic toxicity to a cell; and wherein T1 comprises a targeting ligand that binds to a first target receptor and T2 comprises a second targeting ligand that binds to the second target receptor, which is increased on a tumor cell compared to a normal cell and where the first targeting ligand binds to a targeting receptor selected from the following list:

1 ) a cathepsin type protease

2) a collagenase

3) a gelatinase

4) a matrix metalloproteinase

5) a membrane type matrix metalloproteinase

6) alpha v beta 3 integrin

7) bombesin/gastrin releasing peptide receptors

8) cathepsin B

9) cathepsin D

10) cathepsin K

11) cathepsin L

12) cathepsin O

13) fibroblast activation protein

14) folate binding receptors

15) gastrin/cholecystokinin type B receptor

16) glutamate carboxypeptidase II or (PSMA)

17) guanidinobenzoatase

18) laminin receptor

19) matrilysin or

20) matripase

21) melanocyte stimulating hormone receptor

22) nitrobenzylthioinosine-binding receptors

23) norepenephrine transporters

24) nucleoside transporter proteins

25) peripheral benzodiazepam binding receptors

26) plasmin

27) seprase

28) sigma receptors

29) somatostatin receptors

30) stromelysin 3

31) trypsin

32) urokinase

33) MMP 1

34) MMP 2

35) MMP 3

36) MMP 7

37) MMP9

38) Membrane type matrix metalloproteinase I

39) MMP 12

40) MMP 13

In preferred embodiments; of embodiments (OSTLP #X, for X=1, 2, 3, 4 . . . 40); The effector agent E1 inhibits the denovo synthesis of a biomolecule(s) that is

necessary for cell replication and or survival, and the effector agent E2 inhibits a salvage pathway(s) that can enable a cell to by-pass the metabolic block caused by E1. In a preferred embodiment of these embodiments, E1 inhibits nucleoside synthesis and E2 inhibits nucleoside uptake.

In preferred embodiments of the above embodiments, E1 is comprised of an inhibitor to one or more of the following enxymes:

1.) thymidylate synthase

2.) ribonucleotide reductase

3.) glycinamide ribonucleotide transformylase

4.) 5-aminoimidazole-4-carboxamide ribonucleotide transferase

5.) dihydroorotate dehydrogenase

6.) carbamoyl phosphate synthetase

7.) orotidine-5′-phosphate decarboxylase

8.) inosine 5′monophosphate dehydrogenase

9.) aspartate transcarbamylase

and E2 is comprised of an inhibitor to one or more of the following enzymes:

1.) nucleoside transporter proteins

2.) thymidine kinase

3.) uridine/cytidine kinase

4.) deoxycytidine kinase

5.) deoxyguanosine kinase

6.) hypoxanthine-guanine phosphoribosyltransferase

7.) xanthine-guanine phosphoribosyltransferase

8.) adenine phosphoribosyltransferase

In Preferred Embodiments:

designated: (embodiment “1STLP #.X”, wherein X is the number given below to the pairs of target receptors and X=1, 2, 3, . . . 795);

E1T1 and E2T2 are a set of anti-cancer drugs for use together, wherein E1 and E2 exhibit synergistic toxicity to a cell; and wherein T1 comprises a targeting ligand that binds to the first target receptor (a1); and T2 comprises a second targeting ligand that binds to the second target receptor (a2) indicated in the pairs of (a1 - - - a2) listed below:

1 ) urokinase—a cathepsin type protease;

2) urokinase—a collagenase;

3) urokinase—a gelatinase;

4) urokinase—a matrix metalloproteinase;

5) urokinase—a membrane type matrix metalloproteinase;

6) urokinase—alpha v beta 3 integrin;

7) urokinase—bombesin/gastrin releasing peptide receptors;

8) urokinase—cathepsin B;

9) urokinase—cathepsin D;

10) urokinase—to cathepsin K;

11) urokinase—cathepsin L;

12) urokinase—cathepsin O;

13) urokinase—fibroblast activation protein;

14) urokinase—folate binding receptors;

15) urokinase—gastrin/cholecystokinin type B receptor;

16) urokinase—glutamate carboxypeptidase II or (PSMA);

17) urokinase—guanidinobenzoatase;

18) urokinase—laminin receptor;

19) urokinase—matrilysin;

20) urokinase—matripase;

21) urokinase—melanocyte stimulating hormone receptor;

22) urokinase—nitrobenzylthioinosine-binding receptors or (nucleoside transporter);

23) urokinase—norepinephrine transporters;

24) urokinase—nucleoside transporter proteins;

25) urokinase—peripheral benzodiazepam binding receptors;

26) urokinase—plasmin;

27) urokinase—seprase;

28) urokinase—sigma receptors;

29) urokinase—somatostatin receptors;

30) urokinase—stromelysin 3;

31) urokinase—trypsin;

32) urokinase—urokinase;

33) urokinase—MMP 1;

34) urokinase—MMP 2;

35) urokinase—MMP 3;

36) urokinase—MMP 7;

37) urokinase—MMP 9;

38) urokinase—membrane type matrix metalloproteinase I;

39) urokinase—MMP 12;

40) urokinase—MMP 13;

41) urokinase—a tumor antigen;

42) plasmin—a cathepsin type protease;

43) plasmin—a collagenase;

44) plasmin—a gelatinase;

45) plasmin—a matrix metalloproteinase;

46) plasmin—a membrane type matrix metalloproteinase;

47) plasmin—alpha v beta 3 integrin;

48) plasmin—bombesin/gastrin releasing peptide receptors;

49) plasmin—cathepsin B;

50) plasmin—cathepsin D;

51) plasmin—to cathepsin K;

52) plasmin—cathepsin L;

53) plasmin—cathepsin O;

54) plasmin—fibroblast activation protein;

55) plasmin—folate binding receptors;

56) plasmin—gastrin/cholecystokinin type B receptor;

57) plasmin—glutamate carboxypeptidase II or (PSMA);

58) plasmin—guanidinobenzoatase;

59) plasmin—laminin receptor;

60) plasmin—matrilysin;

61) plasmin—matripase;

62) plasmin—melanocyte stimulating hormone receptor;

63) plasmin—nitrobenzylthioinosine-binding receptors or (nucleoside transporter);

64) plasmin—norepinephrine transporters;

65) plasmin—nucleoside transporter proteins;

66) plasmin—peripheral benzodiazepam binding receptors;

67) plasmin—plasmin;

68) plasmin—seprase;

69) plasmin—sigma receptors;

70) plasmin—somatostatin receptors;

71) plasmin—stromelysin 3;

72) plasmin—trypsin;

73) plasmin—urokinase;

74) plasmin—MMP 1;

75) plasmin—MMP 2;

76) plasmin—MMP 3;

77) plasmin—MMP 7;

78) plasmin—MMP 9;

79) plasmin—membrane type matrix metalloproteinase I;

80) plasmin—MMP 12;

81) plasmin—MMP 13;

82) plasmin—a tumor antigen;

83) a collagenase—a cathepsin type protease;

84) a collagenase—a collagenase;

85) a collagenase—a gelatinase;

86) a collagenase—a matrix metalloproteinase;

87) a collagenase—a membrane type matrix metalloproteinase;

88) a collagenase—alpha v beta 3 integrin;

89) a collagenase—bombesin/gastrin releasing peptide receptors;

90) a collagenase—cathepsin B;

91) a collagenase—cathepsin D;

92) a collagenase—to cathepsin K;

93) a collagenase—cathepsin L;

94) a collagenase—cathepsin O;

95) a collagenase—fibroblast activation protein;

96) a collagenase—folate binding receptors;

97) a collagenase—gastrin/cholecystokinin type B receptor;

98) a collagenase—glutamate carboxypeptidase II or (PSMA);

99) a collagenase—guanidinobenzoatase;

100) a collagenase—laminin receptor;

101) a collagenase—matrilysin;

102) a collagenase—matripase;

103) a collagenase—melanocyte stimulating hormone receptor;

104) a collagenase—nitrobenzylthioinosine-binding receptors or (nucleoside transporter);

105) a collagenase—norepinephrine transporters;

106) a collagenase—nucleoside transporter proteins;

107) a collagenase—peripheral benzodiazepam binding receptors;

108) a collagenase—seprase;

109) a collagenase—sigma receptors;

110) a collagenase—somatostatin receptors;

111) a collagenase—stromelysin 3;

112) a collagenase—trypsin;

113) a collagenase—a collagenase;

114) a collagenase—MMP 1;

115) a collagenase—MMP 2;

116) a collagenase—MMP 3;

117) a collagenase—MMP 7;

118) a collagenase—MMP 9;

119) a collagenase—membrane type matrix metalloproteinase I;

120) a collagenase—MMP 12;

121) a collagenase—MMP 13;

122) a collagenase—a tumor antigen;

123) a gelatinase—a cathepsin type protease;

124) a gelatinase—a gelatinase;

125) a gelatinase—a matrix metalloproteinase;

126) a gelatinase—a membrane type matrix metalloproteinase;

127) a gelatinase—alpha v beta 3 integrin;

128) a gelatinase—bombesin/gastrin releasing peptide receptors;

129) a gelatinase—cathepsin B;

130) a gelatinase—cathepsin D;

131) a gelatinase—to cathepsin K;

132) a gelatinase—cathepsin L;

133) a gelatinase—cathepsin O;

134) a gelatinase—fibroblast activation protein;

135) a gelatinase—folate binding receptors;

136) a gelatinase—gastrin/cholecystokinin type B receptor;

137) a gelatinase—glutamate carboxypeptidase II or (PSMA);

138) a gelatinase—guanidinobenzoatase;

139) a gelatinase—laminin receptor;

140) a gelatinase—matrilysin;

141) a gelatinase—matripase;

142) a gelatinase—melanocyte stimulating hormone receptor;

143) a gelatinase—nitrobenzylthioinosine-binding receptors or (nucleoside transporter);

144) a gelatinase—norepinephrine transporters;

145) a gelatinase—nucleoside transporter proteins;

146) a gelatinase—peripheral benzodiazepam binding receptors;

147) a gelatinase—seprase;

148) a gelatinase—sigma receptors;

149) a gelatinase—somatostatin receptors;

150) a gelatinase—stromelysin 3;

151) a gelatinase—trypsin;

152) a gelatinase—MMP 1;

153) a gelatinase—MMP 2;

154) a gelatinase—MMP 3;

155) a gelatinase—MMP 7;

156) a gelatinase—MMP 9;

157) a gelatinase—membrane type matrix metalloproteinase I;

158) a gelatinase—MMP 12;

159) a gelatinase—MMP 13;

160) a gelatinase—a tumor antigen;

161) a matrix metalloproteinase—a cathepsin type protease;

162) a matrix metalloproteinase—a collagenase;

163) a matrix metalloproteinase—a gelatinase;

164) a matrix metalloproteinase—a matrix metalloproteinase;

165) a matrix metalloproteinase—a membrane type matrix metalloproteinase;

166) a matrix metalloproteinase—alpha v beta 3 integrin;

167) a matrix metalloproteinase—bombesin/gastrin releasing peptide receptors;

168) a matrix metalloproteinase—cathepsin B;

169) a matrix metalloproteinase—cathepsin D;

170) a matrix metalloproteinase—to cathepsin K;

171) a matrix metalloproteinase—cathepsin L;

172) a matrix metalloproteinase—cathepsin O;

173) a matrix metalloproteinase—fibroblast activation protein;

174) a matrix metalloproteinase—folate binding receptors;

175) a matrix metalloproteinase—gastrin/cholecystokinin type B receptor;

176) a matrix metalloproteinase—glutamate carboxypeptidase II or (PSMA);

177) a matrix metalloproteinase—guanidinobenzoatase;

178) a matrix metalloproteinase—laminin receptor;

179) a matrix metalloproteinase—matrilysin;

180) a matrix metalloproteinase—matripase;

181) a matrix metalloproteinase—melanocyte stimulating hormone receptor;

182) a matrix metalloproteinase—nitrobenzylthioinosine-binding receptors or (nucleoside transporter);

183) a matrix metalloproteinase—norepinephrine transporters;

184) a matrix metalloproteinase—nucleoside transporter proteins;

185) a matrix metalloproteinase—peripheral benzodiazepam binding receptors;

186) a matrix metalloproteinase—plasmin;

187) a matrix metalloproteinase—seprase;

188) a matrix metalloproteinase—sigma receptors;

189) a matrix metalloproteinase—somatostatin receptors;

190) a matrix metalloproteinase—stromelysin 3;

191) a matrix metalloproteinase—trypsin;

192) a matrix metalloproteinase—a matrix metalloproteinase;

193) a matrix metalloproteinase—MMP 1;

194) a matrix metalloproteinase—MMP 2;

195) a matrix metalloproteinase—MMP 3;

196) a matrix metalloproteinase—MMP 7;

197) a matrix metalloproteinase—MMP 9;

198) a matrix metalloproteinase—membrane type matrix metalloproteinase I;

199) a matrix metalloproteinase—MMP 12;

200) a matrix metalloproteinase—MMP 13;

201) a matrix metalloproteinase—a tumor antigen;

202) a membrane type metailoproteinase—a cathepsin type protease;

203) a membrane type metalloproteinase—a membrane type matrix metalloproteinase;

204) a membrane type metalloproteinase—alpha v beta 3 integrin;

205) a membrane type metalloproteinase—bombesin/gastrin releasing peptide receptors;

206) a membrane type metalloproteinase—cathepsin B;

207) a membrane type metalloproteinase—cathepsin D;

208) a membrane type metalloproteinase—to cathepsin K;

209) a membrane type metalloproteinase—cathepsin L;

210) a membrane type metalloproteinase—cathepsin O;

211) a membrane type metalloproteinase—fibroblast activation protein;

212) a membrane type metalloproteinase—folate binding receptors;

213) a membrane type metalloproteinase—gastrin/cholecystokinin type B receptor;

214) a membrane type metalloproteinase—glutamate carboxypeptidase II or (PSMA);

215) a membrane type metalloproteinase—guanidinobenzoatase;

216) a membrane type metalloproteinase—laminin receptor;

217) a membrane type metalloproteinase—matrilysin;

218) a membrane type metalloproteinase—matripase;

219) a membrane type metalloproteinase—melanocyte stimulating hormone receptor;

220) a membrane type metalloproteinase—nitrobenzylthioinosine-binding receptors or (nucleoside transporter);

221) a membrane type metalloproteinase—norepinephrine transporters;

222) a membrane type metalloproteinase—nucleoside transporter proteins;

223) a membrane type metalloproteinase—peripheral benzodiazepam binding receptors;

224) a membrane type metalloproteinase—seprase;

225) a membrane type metalloproteinase—sigma receptors;

226) a membrane type metalloproteinase—somatostatin receptors;

227) a membrane type metalloproteinase—stromelysin 3;

228) a membrane type metalloproteinase—trypsin;

229) a membrane type metalloproteinase—MMP 1;

230) a membrane type metalloproteinase—MMP 2;

231) a membrane type metalloproteinase—MMP 3;

232) a membrane type metalloproteinase—MMP 7;

233) a membrane type metalloproteinase—MMP 9;

234) a membrane type metalloproteinase—membrane type matrix metalloproteinase I;

235) a membrane type metalloproteinase—MMP 12;

236) a membrane type metalloproteinase—MMP 13;

237) a membrane type metalloproteinase—a tumor antigen;

238) alpha v beta 3 integrin—a cathepsin type protease;

239) alpha v beta 3 integrin—alpha v beta 3 integrin;

240) alpha v beta 3 integrin—bombesin/gastrin releasing peptide receptors;

241) alpha v beta 3 integrin—cathepsin B;

242) alpha v beta 3 integrin—cathepsin D;

243) alpha V beta 3 integrin—cathepsin K;

244) alpha v beta 3 integrin—cathepsin L;

245) alpha v beta 3 integrin—cathepsin O;

246) alpha v beta 3 integrin—fibroblast activation protein;

247) alpha v beta 3 integrin—folate binding receptors;

248) alpha v beta 3 integrin—gastrin/cholecystokinin type B receptor;

249) alpha v beta 3 integrin—glutamate carboxypeptidase II or (PSMA);

250) alpha v beta 3 integrin—guanidinobenzoatase;

251) alpha v beta 3 integrin—laminin receptor;

252) alpha v beta 3 integrin—matrilysin;

253) alpha v beta 3 integrin—matripase;

254) alpha v beta 3 integrin—melanocyte stimulating hormone receptor;

255) alpha v beta 3 integrin—nitrobenzylthioinosine-binding receptors or (nucleoside transporter);

256) alpha v beta 3 integrin—norepinephrine transporters;

257) alpha v beta 3 integrin—nucleoside transporter proteins;

258) alpha v beta 3 integrin—peripheral benzodiazepam binding receptors;

259) alpha v beta 3 integrin—seprase;

260) alpha v beta 3 integrin—sigma receptors;

261) alpha v beta 3 integrin—somatostatin receptors;

262) alpha v beta 3 integrin—stromelysin 3;

263) alpha v beta 3 integrin—trypsin;

264) alpha v beta 3 integrin—MMP 1;

265) alpha V beta 3 integrin—MMP 2;

266) alpha v beta 3 integrin—MMP 3;

267) alpha v beta 3 integrin—MMP 7;

268) alpha v beta 3 integrin—MMP 9;

269) alpha v beta 3 integrin—membrane type matrix metalloproteinase I;

270) alpha v beta 3 integrin—MMP 12;

271) alpha v beta 3 integrin—MMP 13;

272) alpha v beta 3 integrin—a tumor antigen;

273) cathepsin B—a cathepsin type protease;

274) cathepsin B—bombesin/gastrin releasing peptide receptors;

275) cathepsin B—cathepsin B;

276) cathepsin B—cathepsin D;

277) cathepsin B—to cathepsin K;

278) cathepsin B—cathepsin L;

279) cathepsin B—cathepsin O;

280) cathepsin B—fibroblast activation protein;

281) cathepsin B—folate binding receptors;

282) cathepsin B—gastrin/cholecystokinin type B receptor;

283) cathepsin B—glutamate carboxypeptidase II or (PSMA);

284) cathepsin B—guanidinobenzoatase;

285) cathepsin B—laminin receptor;

286) cathepsin B—matrilysin;

287) cathepsin B—matripase;

288) cathepsin B—melanocyte stimulating hormone receptor;

289) cathepsin B—nitrobenzylthioinosine-binding receptors or (nucleoside transporter);

290) cathepsin B—norepinephrine transporters;

291) cathepsin B—nucleoside transporter proteins;

292) cathepsin B—peripheral benzodiazepam binding receptors;

293) cathepsin B—seprase;

294) cathepsin B—sigma receptors;

295) cathepsin B—somatostatin receptors;

296) cathepsin B—stromelysin 3;

297) cathepsin B—trypsin;

298) cathepsin B—MMP 1;

299) cathepsin B—MMP 2;

300) cathepsin B—MMP 3;

301) cathepsin B—MMP 7;

302) cathepsin B—MMP 9;

303) cathepsin B—membrane type matrix metalloproteinase I;

304) cathepsin B—MMP 12;

305) cathepsin B—MMP 13;

306) cathepsin B—a tumor antigen;

307) bombesin/gastrin releasing peptide receptors a cathepsin type protease;

308) bombesin/gastrin releasing peptide receptors—bombesin/gastrin releasing peptide receptors;

309) bombesin/gastrin releasing peptide receptors cathepsin B;

310) bombesin/gastrin releasing peptide receptors—cathepsin D;

311) bombesin/gastrin releasing peptide receptors—to cathepsin K;

312) bombesin/gastrin releasing peptide receptors—cathepsin L;

313) bombesin/gastrin releasing peptide receptors—cathepsin O;

314) bombesin/gastrin releasing peptide receptors—fibroblast activation protein;

315) bombesin/gastrin releasing peptide receptors—folate binding receptors;

316) bombesin/gastrin releasing peptide receptors—gastrin/cholecystokinin type B receptor;

317) bombesin/gastrin releasing peptide receptors—glutamate carboxypeptidase II or (PSMA);

318) bombesin/gastrin releasing peptide receptors—guanidinobenzoatase;

319) bombesin/gastrin releasing peptide receptors—laminin receptor;

320) bombesin/gastrin releasing peptide receptors—matrilysin;

321) bombesin/gastrin releasing peptide receptors—matripase;

322) bombesin/gastrin releasing peptide receptors—melanocyte stimulating hormone receptor;

323) bombesin/gastrin releasing peptide receptors—nitrobenzylthioinosine-binding receptors or (nucleoside transporter);

324) bombesin/gastrin releasing peptide receptors—norepinephrine transporters;

325) bombesin/gastrin releasing peptide receptors—nucleoside transporter proteins;

326) bombesin/gastrin releasing peptide receptors—peripheral benzodiazepam binding receptors;

327) bombesin/gastrin releasing peptide receptors—seprase;

328) bombesin/gastrin releasing peptide receptors—sigma receptors;

329) bombesin/gastrin releasing peptide receptors—somatostatin receptors;

330) bombesin/gastrin releasing peptide receptors—stromelysin 3;

331) bombesin/gastrin releasing peptide receptors—trypsin;

332) bombesin/gastrin releasing peptide receptors—MMP 1;

333) bombesin/gastrin releasing peptide receptors—MMP 2;

334) bombesin/gastrin releasing peptide receptors—MMP 3;

335) bombesin/gastrin releasing peptide receptors—MMP 7;

336) bombesin/gastrin releasing peptide receptors—MMP 9;

337) bombesin/gastrin releasing peptide receptors—membrane type matrix metalloproteinase I;

338) bombesin/gastrin releasing peptide receptors—MMP 12;

339) bombesin/gastrin releasing peptide receptors—MMP 13;

340) bombesin/gastrin releasing peptide receptors—a tumor antigen;

341) fibroblast activation protein—a cathepsin type protease;

342) fibroblast activation protein—cathepsin D;

343) fibroblast activation protein—to cathepsin K;

344) fibroblast activation protein—cathepsin L;

345) fibroblast activation protein—cathepsin O;

346) fibroblast activation protein—fibroblast activation protein;

347) fibroblast activation protein—folate binding receptors;

348) fibroblast activation protein—gastrin/cholecystokinin type B receptor;

349) fibroblast activation protein—glutamate carboxypeptidase II or (PSMA);

350) fibroblast activation protein—guanidinobenzoatase;

351) fibroblast activation protein—laminin receptor;

352) fibroblast activation protein—matrilysin;

353) fibroblast activation protein—matripase;

354) fibroblast activation protein—melanocyte stimulating hormone receptor;

355) fibroblast activation protein—nitrobenzylthioinosine-binding receptors or (nucleoside transporter);

356) fibroblast activation protein—norepinephrine transporters;

357) fibroblast activation protein—nucleoside transporter proteins;

358) fibroblast activation protein—peripheral benzodiazepam binding receptors;

359) fibroblast activation protein—plasmin;

360) fibroblast activation protein—seprase;

361) fibroblast activation protein sigma receptors;

362) fibroblast activation protein—somatostatin receptors;

363) fibroblast activation protein—stromelysin 3;

364) fibroblast activation protein—trypsin;

365) fibroblast activation protein—MMP 1;

366) fibroblast activation protein—MMP 2;

367) fibroblast activation protein—MMP 3;

368) fibroblast activation protein—MMP 7;

369) fibroblast activation protein—MMP 9;

370) fibroblast activation protein—membrane type matrix metalloproteinase I;

371) fibroblast activation protein—MMP 12;

372) fibroblast activation protein—MMP 13;

373) fibroblast activation protein—a tumor antigen;

374) glutamate carboxypeptidase II or PSMA—cathepsin D;

375) glutamate carboxypeptidase II or PSMA—to cathepsin K;

376) glutamate carboxypeptidase II or PSMA—cathepsin L;

377) glutamate carboxypeptidase II or PSMA—cathepsin O;

378) glutamate carboxypeptidase II or PSMA—fibroblast activation protein;

379) glutamate carboxypeptidase II or PSMA—folate binding receptors;

380) glutamate carboxypeptidase II or PSMA—gastrin/cholecystokin in type B receptor;

381) glutamate carboxypeptidase II or PSMA glutamate carboxypeptidase II or (PSMA);

382) glutamate carboxypeptidase II or PSMA—guanidinobenzoatase;

383) glutamate carboxypeptidase II or PSMA—laminin receptor;

384) glutamate carboxypeptidase II or PSMA—matrilysin;

385) glutamate carboxypeptidase II or PSMA—matripase;

386) glutamate carboxypeptidase II or PSMA—melanocyte stimulating hormone receptor;

387) glutamate carboxypeptidase II or PSMA—nitrobenzylthioinosine-binding receptors or (nucleoside transporter);

388) glutamate carboxypeptidase II or PSMA—nucleoside transporter proteins;

389) glutamate carboxypeptidase II or PSMA—peripheral benzodiazepam binding receptors;

390) glutamate carboxypeptidase II or PSMA—seprase;

391) glutamate carboxypeptidase II or PSMA—sigma receptors;

392) glutamate carboxypeptidase II or PSMA—somatostatin receptors;

393) glutamate carboxypeptidase II or PSMA—stromelysin 3;

394) glutamate carboxypeptidase II or PSMA—trypsin;

395) glutamate carboxypeptidase II or PSMA—MMP 1;

396) glutamate carboxypeptidase II or PSMA—MMP 2;

397) glutamate carboxypeptidase II or PSMA—MMP 3;

398) glutamate carboxypeptidase II or PSMA—MMP 7;

399) glutamate carboxypeptidase II or PSMA—MMP 9;

400) glutamate carboxypeptidase II or PSMA—membrane type matrix metalloproteinase I;

401) glutamate carboxypeptidase II or PSMA—MMP 12;

402) glutamate carboxypeptidase II or PSMA—MMP 13;

403) glutamate carboxypeptidase II or PSMA—a tumor antigen;

404) laminin receptor—a cathepsin type protease;

405) laminin receptor—cathepsin B;

406) laminin receptor—cathepsin D;

407) laminin receptor—to cathepsin K;

408) laminin receptor—cathepsin L;

409) laminin receptor—cathepsin O;

410) laminin receptor—fibroblast activation protein;

411) laminin receptor—folate binding receptors;

412) laminin receptor—gastrin/cholecystokinin type B receptor;

413) laminin receptor—guanidinobenzoatase;

414) laminin receptor—laminin receptor;

415) laminin receptor—matrilysin;

416) laminin receptor—matripase;

417) laminin receptor—melanocyte stimulating hormone receptor;

418) laminin receptor—nitrobenzylthioinosine-binding receptors or (nucleoside transporter);

419) laminin receptor—norepinephrine transporters;

420) laminin receptor—nucleoside transporter proteins;

421) laminin receptor—peripheral benzodiazepam binding receptors;

422) laminin receptor—seprase;

423) laminin receptor—sigma receptors;

424) laminin receptor—somatostatin receptors;

425) laminin receptor—stromelysin 3;

426) laminin receptor—trypsin;

427) laminin receptor—MMP 1;

428) laminin receptor—MMP 2;

429) laminin receptor—MMP 3;

430) laminin receptor—MMP 7;

431) laminin receptor—MMP 9;

432) laminin receptor—membrane type matrix metalloproteinase I;

433) laminin receptor—MMP 12;

434) laminin receptor—MMP 13;

435) laminin receptor—a tumor antigen;

436) seprase—a cathepsin type protease;

437) seprase—cathepsin D;

438) seprase—to cathepsin K;

439) seprase—cathepsin L;

440) seprase—cathepsin O;

441) seprase—fibroblast activation protein;

442) seprase—folate binding receptors;

443) seprase—gastrin/cholecystokinin type B receptor;

444) seprase—guanidinobenzoatase;

445) seprase—matripase;

446) seprase—melanocyte stimulating hormone receptor;

447) seprase—nitrobenzylthioinosine-binding receptors or (nucleoside transporter);

448) seprase—norepinephrine transporters;

449) seprase—nucleoside transporter proteins;

450) seprase—peripheral benzodiazepam binding receptors;

451) seprase—seprase;

452) seprase—sigma receptors;

453) seprase—somatostatin receptors;

454) seprase—stromelysin 3;

455) seprase—trypsin;

456) seprase—MMP 1;

457) seprase—MMP 2;

458) seprase—MMP 3;

459) seprase—MMP 7;

460) seprase—MMP 9;

461) seprase—membrane type matrix metalloproteinase I;

462) seprase—MMP 12;

463) seprase—MMP 13;

464) seprase—a tumor antigen;

465) guanidinobenzoatase—a cathepsin type protease;

466) guanidinobenzoatase—cathepsin D;

467) guanidinobenzoatase—to cathepsin K;

468) guanidinobenzoatase —-cathepsin L;

469) guanidinobenzoatase—cathepsin O;

470) guanidinobenzoatase—fibroblast activation protein;

471) guanidinobenzoatase—folate binding receptors;

472) guanidinobenzoatase—gastrin/cholecystokinin type B receptor;

473) guanidinobenzoatase—guanidinobenzoatase;

474) guanidinobenzoatase—matripase;

475) guanidinobenzoatase—melanocyte stimulating hormone receptor;

476) guanidinobenzoatase—nitrobenzylthioinosine-binding receptors or (nucleoside transporter);

477) guanidinobenzoatase—norepinephrine transporters;

478) guanidinobenzoatase—nucleoside transporter proteins;

479) guanidinobenzoatase—peripheral benzodiazepam binding receptors;

480) guanidinobenzoatase—sigma receptors;

481) guanidinobenzoatase—somatostatin receptors;

482) guanidinobenzoatase—stromelysin 3;

483) guanidinobenzoatase—trypsin;

484) guanidinobenzoatase—MMP 1;

485) guanidinobenzoatase—MMP 2;

486) guanidinobenzoatase—MMP 3;

487) guanidinobenzoatase—MMP 7;

488) guanidinobenzoatase—MMP 9;

489) guanidinobenzoatase—membrane type matrix metalloproteinase I;

490) guanidinobenzoatase—MMP 12;

491) guanidinobenzoatase—MMP 13;

492) guanidinobenzoatase—a tumor antigen;

493) peripheral benzodiazepam binding receptors—a cathepsin type protease;

494) peripheral benzodiazepam binding receptors—cathepsin D;

495) peripheral benzodiazepam binding receptors—to cathepsin K;

496) peripheral benzodiazepam binding receptors—cathepsin L;

497) peripheral benzodiazepam binding receptors—cathepsin O;

498) peripheral benzodiazepam binding receptors—fibroblast activation protein;

499) peripheral benzodiazepam binding receptors—folate binding receptors;

500) peripheral benzodiazepam binding receptors—gastrin/cholecystokinin type B receptor;

501) peripheral benzodiazepam binding receptors—guanidinobenzoatase;

502) peripheral benzodiazepam binding receptors—matripase;

503) peripheral benzodiazepam binding receptors—melanocyte stimulating hormone receptor;

504) peripheral benzodiazepam binding receptors—nitrobenzylthioinosine-binding receptors or (nucleoside transporter);

505) peripheral benzodiazepam binding receptors—norepinephrine transporters;

506) peripheral benzodiazepam binding receptors—nucleoside transporter proteins;

507) peripheral benzodiazepam binding receptors—peripheral benzodiazepam binding receptors;

508) peripheral benzodiazepam binding receptors—sigma receptors;

509) peripheral benzodiazepam binding receptors—somatostatin receptors;

510) peripheral benzodiazepam binding receptors stromelysin 3;

511) peripheral benzodiazepam binding receptors—trypsin;

512) peripheral benzodiazepam binding receptors—MMP 1;

513) peripheral benzodiazepam binding receptors—MMP 2;

514) peripheral benzodiazepam binding receptors—MMP 3;

515) peripheral benzodiazepam binding receptors—MMP 7;

516) peripheral benzodiazepam binding receptors—MMP 9;

517) peripheral benzodiazepam binding receptors—membrane type matrix metalloproteinase I;

518) peripheral benzodiazepam binding receptors—MMP 12;

519) peripheral benzodiazepam binding receptors—MMP 13;

520) peripheral benzodiazepam binding receptors—a tumor antigen;

521) folate binding receptors—a cathepsin type protease;

522) folate binding receptors—cathepsin D;

523) folate binding receptors—to cathepsin K;

524) folate binding receptors—cathepsin L;

525) folate binding receptors—cathepsin O;

526) folate binding receptors—fibroblast activation protein;

527) folate binding receptors—folate binding receptors;

528) folate binding receptors—matripase;

529) folate binding receptors—melanocyte stimulating hormone receptor;

530) folate binding receptors—nitrobenzylthioinosine-binding receptors or (nucleoside transporter);

531) folate binding receptors—norepinephrine transporters;

532) folate binding receptors—nucleoside transporter proteins;

533) folate binding receptors —-sigma receptors;

534) folate binding receptors—somatostatin receptors;

535) folate binding receptors—stromelysin 3;

536) folate binding receptors—trypsin;

537) folate binding receptors—MMP 1;

538) folate binding receptors—MMP 2;

539) folate binding receptors—MMP 3;

540 ) folate binding receptors—MMP 7;

541) folate binding receptors—MMP 9;

542) folate binding receptors—membrane type matrix metalloproteinase I;

543) folate binding receptors—MMP 12;

544) folate binding receptors—MMP 13;

545) folate binding receptors—a tumor antigen;

546) folate binding receptors—a cathepsin type protease;

547) folate binding receptors—cathepsin D;

548) folate binding receptors—to cathepsin K;

549) folate binding receptors—cathepsin L;

550) folate binding receptors—cathepsin O;

551) folate binding receptors—fibroblast activation protein;

552) folate binding receptors—folate binding receptors;

553) folate binding receptors—matripase;

554) folate binding receptors—melanocyte stimulating hormone receptor;

555) folate binding receptors—nitrobenzylthioinosine-binding receptors or (nucleoside transporter);

556) folate binding receptors—norepinephrine transporters;

557) folate binding receptors—nucleoside transporter proteins;

558) folate binding receptors—sigma receptors;

559) folate binding receptors—somatostatin receptors;

560) folate binding receptors—stromelysin 3;

561) folate binding receptors—trypsin;

562) folate binding receptors—MMP 1;

563) folate binding receptors—MMP 2;

564) folate binding receptors—MMP 3;

565) folate binding receptors—MMP 7;

566) folate binding receptors—MMP 9;

567) folate binding receptors—membrane type matrix metalloproteinase I;

568) folate binding receptors—MMP 12;

569) folate binding receptors—MMP 13;

570) folate binding receptors—a tumor antigen;

571) nucleoside transporter proteins—a cathepsin type protease;

572) nucleoside transporter proteins—cathepsin D;

573) nucleoside transporter proteins—to cathepsin K;

574) nucleoside transporter proteins—cathepsin L;

575) nucleoside transporter proteins—cathepsin O;

576) nucleoside transporter proteins—fibroblast activation protein;

577) nucleoside transporter proteins—nucleoside transporter proteins;

578) nucleoside transporter proteins—matripase;

579) nucleoside transporter proteins—melanocyte stimulating hormone receptor;

580) nucleoside transporter proteins—nitrobenzylthioinosine-binding receptors or (nucleoside transporter);

581) nucleoside transporter proteins—norepinephrine transporters;

582) nucleoside transporter proteins—nucleoside transporter proteins;

583) nucleoside transporter proteins—sigma receptors;

584) nucleoside transporter proteins—somatostatin receptors;

585) nucleoside transporter proteins—stromelysin 3;

586) nucleoside transporter proteins—trypsin;

587) nucleoside transporter proteins—MMP 1;

588) nucleoside transporter proteins—MMP 2;

589) nucleoside transporter proteins—MMP 3;

590) nucleoside transporter proteins—MMP 7;

591) nucleoside transporter proteins—MMP 9;

592) nucleoside transporter proteins—membrane type matrix metalloproteinase I;

593) nucleoside transporter proteins—MMP 12;

594) nucleoside transporter proteins—MMP 13;

595) nucleoside transporter proteins—a tumor antigen;

596) melanocyte stimulating hormone receptor—a cathepsin type protease;

597) melanocyte stimulating hormone receptor—cathepsin D;

598) melanocyte stimulating hormone receptor—to cathepsin K;

599) melanocyte stimulating hormone receptor—cathepsin L;

600) melanocyte stimulating hormone receptor—cathepsin O;

601) melanocyte stimulating hormone receptor—fibroblast activation protein;

602) melanocyte stimulating hormone receptor—melanocyte stimulating hormone receptor;

603) melanocyte stimulating hormone receptor—melanocyte stimulating hormone receptor;

604) melanocyte stimulating hormone receptor—nitrobenzylthioinosine-binding receptors or (nucleoside transporter);

605) melanocyte stimulating hormone receptor—norepinephrine transporters;

606) melanocyte stimulating hormone receptor—nucleoside transporter proteins;

607) melanocyte stimulating hormone receptor—sigma receptors;

608) melanocyte stimulating hormone receptor—somatostatin receptors;

609) melanocyte stimulating hormone receptor—stromelysin 3;

610) melanocyte stimulating hormone receptor—trypsin;

611) melanocyte stimulating hormone receptor—MMP 1;

612) melanocyte stimulating hormone receptor—MMP 2;

613) melanocyte stimulating hormone receptor—MMP 3;

614) melanocyte stimulating hormone receptor—MMP 7;

615) melanocyte stimulating hormone receptor—MMP 9;

616) melanocyte stimulating hormone receptor—membrane type matrix metalloproteinase I;

617) melanocyte stimulating hormone receptor—MMP 12;

618) melanocyte stimulating hormone receptor—MMP 13;

619) melanocyte stimulating hormone receptor—a tumor antigen;

620) sigma receptors—a cathepsin type protease;

621) sigma receptors—cathepsin D;

622) sigma receptors—to cathepsin K;

623) sigma receptors—cathepsin L;

624) sigma receptors—cathepsin O;

625) sigma receptors—fibroblast activation protein;

626) sigma receptors—sigma receptors;

627) sigma receptors—matripase;

628) sigma receptors—norepinephrine transporters;

629) sigma receptors—sigma receptors;

630) sigma receptors—somatostatin receptors;

631) sigma receptors—stromelysin 3;

632) sigma receptors—trypsin;

633) sigma receptors—MMP 1;

634) sigma receptors—MMP 2;

635) sigma receptors—MMP 3;

636) sigma receptors—MMP 7;

637) sigma receptors—MMP 9;

638) sigma receptors—membrane type matrix metalloproteinase I;

639) sigma receptors—MMP 12;

640) sigma receptors—MMP 13;

641) sigma receptors—a tumor antigen;

642) somatostatin receptors—a cathepsin type protease;

643) somatostatin receptors—cathepsin D;

644) somatostatin receptors—to cathepsin K;

645) somatostatin receptors—cathepsin L;

646) somatostatin receptors—cathepsin O;

647) somatostatin receptors—fibroblast activation protein;

648) somatostatin receptors—somatostatin receptors;

649) somatostatin receptors—matripase;

650) somatostatin receptors—melanocyte stimulating hormone receptor;

651) somatostatin receptors—sigma receptors;

652) somatostatin receptors—somatostatin receptors;

653) somatostatin receptors—stromelysin 3;

654) somatostatin receptors—trypsin;

655) somatostatin receptors—MMP 1;

656) somatostatin receptors—MMP 2;

657) somatostatin receptors—MMP 3;

658) somatostatin receptors—MMP 7;

659) somatostatin receptors—MMP 9;

660) somatostatin receptors—membrane type matrix metalloproteinase I;

661) somatostatin receptors—MMP 12;

662) somatostatin receptors—MMP 13;

663) somatostatin receptors—a tumor antigen;

664) stromelysin 3—a cathepsin type protease;

665) stromelysin 3—cathepsin D;

666) stromelysin 3—to cathepsin K;

667) stromelysin 3—cathepsin L;

668) stromelysin 3—cathepsin 0;

669) stromelysin 3—fibroblast activation protein;

670) stromelysin 3—stromelysin 3;

671) stromelysin 3—matripase;

672) stromelysin 3—melanocyte stimulating hormone receptor;

673) stromelysin 3—somatostatin receptors;

674) stromelysin 3—trypsin;

675) stromelysin 3—MMP 1;

676) stromelysin 3—MMP 2;

677) stromelysin 3—MMP 3;

678) stromelysin 3—MMP7;

679) stromelysin 3—MMP 9;

680) stromelysin 3—membrane type matrix metalloproteinase I;

681) stromelysin 3—MMP 12;

682) stromelysin 3—MMP 13;

683) stromelysin 3—a tumor antigen;

684) trypsin—a cathepsin type protease;

685) trypsin—cathepsin D;

686) trypsin—to cathepsin K;

687) trypsin—cathepsin L;

688) trypsin—cathepsin O;

689) trypsin—fibroblast activation protein;

690) trypsin—trypsin;

691) trypsin—matripase;

692) trypsin—melanocyte stimulating hormone receptor;

693) trypsin—stromelysin 3;

694) trypsin—MMP 1;

695) trypsin—MMP 2;

696) trypsin—MMP 3;

697) trypsin—MMP 7;

698) trypsin—MMP 9;

699) trypsin—membrane type matrix metalloproteinase I;

700) trypsin—MMP 12;

701) trypsin—MMP 13;

702) trypsin—a tumor antigen;

703) MMP 1—a cathepsin type protease;

704) MMP 1—cathepsin D;

705) MMP 1—to cathepsin K;

706) MMP 1—cathepsin L;

707) MMP 1—cathepsin O;

708) MMP 1—fibroblast activation protein;

709) MMP 1—matripase;

710) MMP 1—melanocyte stimulating hormone receptor;

711) MMP 1—stromelysin 3;

712) MMP 1—MMP1;

713) MMP 1—MMP 2;

714) MMP 1—MMP 3;

715) MMP 1—MMP 7;

716) MMP 1—MMP9;

717) MMP 1—membrane type matrix metalloproteinase I;

718) MMP 1—MMP 12;

719) MMP 1—MMP 13;

720) MMP 1—a tumor antigen;

721) MMP-2—a cathepsin type protease;

722) MMP-2—cathepsin D;

723) MMP-2—to cathepsin K;

724) MMP-2—cathepsin L;

725) MMP-2 —-cathepsin O;

726) MMP-2—fibroblast activation protein;

727) MMP-2—matripase;

728) MMP-2—melanocyte stimulating hormone receptor;

729) MMP-2—stromelysin 3;

730) MMP-2—MMP2;

731) MMP-2—MMP3;

732) MMP-2—MMP 7;

733) MMP-2—MMP 9;

734) MMP-2—membrane type matrix metalloproteinase I;

735) MMP-2—MMP-2;

736) MMP-2—MMP-3;

737) MMP-2—a tumor antigen;

738) MMP-3—a cathepsin type protease;

739) MMP-3—cathepsin D;

740) MMP-3—to cathepsin K;

741) MMP-3—cathepsin L;

742) MMP-3—cathepsin O;

743) MMP-3—matripase;

744) MMP-3—MMP 3;

745) MMP-3—MMP 7;

746) MMP-3—MMP 9;

747) MMP-3—membrane type matrix metalloproteinase I;

748) MMP-3—MMP-3;

749) MMP-3—a tumor antigen;

750) MMP 7—a cathepsin type protease;

751) MMP 7—cathepsin D;

752) MMP 7—to cathepsin K;

753) MMP 7—cathepsin L;

754) MMP 7—cathepsin O;

755) MMP 7—fibroblast activation protein;

756) MMP 7—matripase;

757) MMP 7—stromelysin 3;

758) MMP 7—MMP7;

759) MMP 7—MMP 9;

760) MMP 7—membrane type matrix metalloproteinase I;

761) MMP 7—a tumor antigen;

762) MMP 9—a cathepsin type protease;

763) MMP 9—cathepsin D;

764) MMP 9—to cathepsin K;

765) MMP 9—cathepsin L;

766) MMP 9—cathepsin O;

767) MMP 9—matripase;

768) MMP 9—MMP9;

769) MMP 9—membrane type matrix metalloproteinase I;

770) MMP 9—a tumor antigen;

771) MMP 12—a cathepsin type protease;

772) MMP 12—cathepsin D;

773) MMP 12—to cathepsin K;

774) MMP 12—cathepsin L;

775) MMP 12—cathepsin O;

776) MMP 12—matripase;

777) MMP 12—MMP 2;

778) MMP 12—membrane type matrix metalloproteinase I;

779) MMP 12—a tumor antigen;

780) MMP 13—a cathepsin type protease;

781) MMP 13—cathepsin D;

782) MMP 13—to cathepsin K;

783) MMP 13—cathepsin L;

784) MMP 13—cathepsin O;

785) MMP 13—matripase;

786) MMP 13—membrane type matrix metalloproteinase I;

787) MMP 13—a tumor antigen;

788) Membrane type matrix metalloproteinase—a cathepsin type protease;

789) Membrane type matrix metalloproteinase cathepsin D;

790) Membrane type matrix metalloproteinase—to cathepsin K;

791) Membrane type matrix metalloproteinase—cathepsin L;

792) Membrane type matrix metalloproteinase—cathepsin O;

793) Membrane type matrix metalloproteinase—matripase;

794) Membrane type matrix metalloproteinase—membrane type matrix metalloproteinase I;

795) and Membrane type matrix metalloproteinase—a tumor antigen.

In preferred embodiments of (embodiment “1STLP #.X”, for X=1, 2, 3, . . . 795); E1 is an inhibitor of thymidylate synthase and E2 is an inhibitor of nucleoside transporter proteins.

In preferred embodiments of (embodiment “1STLP #.X”, for X=1, 2, 3, . . . 795); E1 is an embodiment of 1E1.1, and E2 is an embodiment of 1 E2.1.

In preferred embodiments of (embodiment “1STLP #.X”, for X=1, 2, 3, . . . 795); E1 is an embodiment of 1E1.1, and E2is an embodiment of 1E2.2.

In preferred embodiments of (embodiment “1STLP #.X”, for X=1, 2, 3, . . . 795); E1 is an embodiment of 1E1.1,and E2 is an embodiment of 1E2.3.

In preferred embodiments of (embodiment “LSTLP #.X”, for X=1, 2, 3, . . . 795); E1 is an embodiment of 1E1.1, and E2 is an embodiment of 1E2.4.

In preferred embodiments of (embodiment “LSTLP #.X”, for X=1, 2, 3, . . . 795); E1 is an embodiment of 2E1.1, and E2 is an embodiment of 2E2.1.

In preferred embodiments of (embodiment “lSTLP #.X”, for X=1, 2, 3, . . . 795); E1 is an embodiment of 2E1.1,and E2 is an embodiment of 2E2.2.

In preferred embodiments of (embodiment “1STLP #.X”, for X=1, 2, 3, . . . 795); E1 is an embodiment of 2E1.1, and E2 is an embodiment of 2E2.3.

In preferred embodiments of (embodiment “1STLP #.X”, for X=1, 2, 3, . . . 795); E1 is an embodiment of 3E1.1, and E2 is an embodiment of 3E2.1.

In preferred embodiments of (embodiment “1STLP #.X”, for X=1, 2, 3, . . . 795); E1 is an embodiment of 3E1.2, and E2 is an embodiment of 3E2.1.

In preferred embodiments of (embodiment “1STLP #.X”, for X=1, 2, 3, . . . 795); E1 is an embodiment of 3E1.2, and E2is an embodiment of 1E2.1.

In preferred embodiments of (embodiment “1STLP #.X”, for X=1, 2, 3, . . . 795); E1 is an embodiment of 3E1.2, and E2is an embodiment of 1E2.2.

In preferred embodiments of (embodiment “1STLP #.X”, for X=1, 2, 3, . . . 795); E1 is an embodiment of 3E1.2, and E2 is an embodiment of 1E2.3.

In preferred embodiments of (embodiment “1STLP #.X”, for X=1, 2, 3, . . . 795); E1 is an embodiment of 3E1.2, and E2 is an embodiment of 1E2.4.

In preferred embodiments of (embodiment “1STLP #.X”, for X=1, 2, 3, . . . 795); E1 is an embodiment of 4E1.1, and E2is an embodiment of 1E2.1.

In preferred embodiments of (embodiment “1STLP #.X”, for X=1, 2, 3, . . . 795); E1 is an embodiment of 4E1.1, and E2is an embodiment of 1E2.2.

In preferred embodiments of (embodiment “1STLP #.X”, for X=1, 2, 3, . . . 795); E1 is an embodiment of 4E1.l, and E2 is an embodiment of 1E2.3.

In preferred embodiments of (embodiment “1STLP #.X”, for X=1, 2, 3, . . . 795); E1 is an embodiment of 4E1.1, and E2 is an embodiment of 1E2.4.

In preferred embodiments of (embodiment “1STLP #.X”, for X=1, 2, 3, . . . 795); E1 is an embodiment of 5E1.1, and E2is an embodiment of 1E2.1.

In preferred embodiments of (embodiment “1STLP #.X”, for X=1, 2, 3, . . . 795); E1 is an embodiment of 5E1.1, and E2is an embodiment of 1E2.2.

In preferred embodiments of (embodiment “1STLP #.X”, for X=1, 2, 3, . . . 795); E1 is an embodiment of 5E1.1, and E2 is an embodiment of 1E2.3.

In preferred embodiments of (embodiment “1STLP #.X”, for X=1, 2, 3, . . . 795); E1 is an embodiment of 5E1.1, and E2 is an embodiment of 1E2.4.

In preferred embodiments of (embodiment “1STLP #.X”, for X=1, 2, 3, . . . 795); E1 is an embodiment of 6E1.1, and E2 is an embodiment of 1E2.1.

In preferred embodiments of (embodiment “1STLP #.X”, for X=1, 2, 3, . . . 795); E1 is an embodiment of 6E1.1, and E2is an embodiment of 1E2.2.

In preferred embodiments of (embodiment “1STLP #.X”, for X=1, 2, 3, . . . 795); E1 is an embodiment of 6E1.1, and E2 is an embodiment of 1E2.3.

In preferred embodiments of (embodiment “1STLP #.X”, for X=1, 2, 3, . . . 795); E1 is an embodiment of 6E1.1, and E2 is an embodiment of 1E2.4.

In a preferred embodiment ET is an anti-cancer drug comprised of at least one tumor-selective targeting ligand a masked effector agent that can stimulate the innate immune system and that can be unmasked at the tumor.

In a preferred embodiment ET is an anti-cancer drug comprised of at least one tumor-selective targeting ligand one or more masked effector agents that can stimulate the innate immune system wherein that effector agent when unmasked comprises a:

1.) N-formyl peptide receptor agonists

2.) Tuftsin receptor agonists

3.) Lipoxin A(4) receptor agonists

4.) Leukotriene B4 agonists

5.) 3-formyl-1 -butyl-pyrophosphates receptor agonists

6.) Gal alpha(1,3)Gal. analogs

In a preferred embodiment of the above embodiment, ET is also comprised of a second group which can irreversibly modify a biomolecule that is over-expressed at the tumor. In a preferred embodiment ET is also comprised of two targeting ligands of (embodiments TLP #.X, wherein X=1, 2, 3, . . . 795).

A preferred embodiment of the present invention comprises a compound with a masked intracellular transport ligand.

A preferred embodiment of the present invention is a method of targeting an immune response against a tumor which is comprised of the following steps:

1.) Sensitizing the patient against a set of neoantigens, and

2.) Administering, to the patient, a compound that interacts with tumor components and thereby generates the neoantigens in the tumor.

A preferred embodiment, of the above method is comprised of administering to the patient a compound ET, wherein ET is comprised of a targeting legand that binds to a targeting receptor present at increased amounts at the tumor and an effector agent E that irreversibly chemically modifies a biomolecule that is increased at the tumor. Numerous examples of suitable compounds ET for this purpose are given in this document. In preferred embodiments of the above, the neoantigens are derived from one or more of the following:

1.) Prostate specific Antigen

2.) Human glandular kallikrein 2

3.) Prostatic acid phosphatase

4.) Plasmin

5.) Placental type alkaline phosphatase

6.) Matriptase

7.) Matrix metalloproteinases

8.) Thymidine phosphorylase

9.) Trypsin

10.) Urokinase

11.) Fatty Acid Synthase

12.) Steroid sulfatase

13.) Epidermal growth factor receptor

14.) Mitogen activated protein kinase kinase

15.) Phosphatidylinositol 3-kinase

16.) Mitogen activated protein kinase

17.) Thymidylate synthase

18.) Protein kinase A

19.) Fibroblast activation protein/seprase

20.) P-glycoprotein

A preferred embodiment of the present invention is a method for generating neoantigens (AG) from a target receptor (rn) by contacting the target receptor with a compound E-T in which E includes the structure: RN-L-V, wherein RN is a group that binds with high affinity to the target rn, L is a linker, and V is a group that can covalently modify the target rn; and wherein RN and V are linked together in a manner so as to allow RN to retain binding affinity to rn and V to functionally modify rn; and wherein T is a targeting agent. In a preferred embodiment V is a free radical generator and modifies rn by the production of free radicals.

A preferred embodiment of the present invention is a method of generating neoantigens comprised of contacting a tumor with a compound ET which is comprised of a tumor-selective targeting ligand wherein E is an effector agent comprised of an irreversible enzyme inhibitor. In a preferred embodiment E is a mechanism based suicide inhibitor for a target enzyme and the neoantigens are derived from said enzyme. In a preferred embodiment said enzyme is overexpresed at tumor cells. In a preferred embodiment E is a mechanism based suicide inhibitor for PSA. In a preferred embodiment E is a mechanism based suicide inhibitor or irreversible inhibitor for Prostate Specific Antigen, or Human glandular kallikrein 2, or Prostatic acid phosphatase, or Plasmin, or Matriptase, or A Matrix metalloproteinases, Trypsin, or Urokinase, or Fatty Acid Synthase, or Steroid sulfatase, or Epidermal growth factor receptors, or Mitogen activated protein kinase kinase, or Phosphatidylinositol 3-kinase, or Mitogen activated protein kinase, or an Estrogen receptor, or Thymidylate synthase, or Protein kinase A, or Fibroblast activation protein or seprase, or P-glycoprotein, or Ribonucleotide diphosphate reductase, or Dihydrofolate reductase, or Src Kinases, or Platelet-derived growth factor receptors, or MMP 7, or MMP 1, or MMP 2, or MMP 3, or MMP 9, or MMP 12, or MMP 13, or Membrane type MMP 1, or A Cathepsin, or Cathepsin B, or Glutathione S -Transferases.

A preferred embodiment of the present invention is a method of treating a patient with prostate cancer which is comprised of the following steps:

a. Sensitizing the patient against a set of neoantigens derived from PSA

b. Administering, to the patient, a compound that interacts with PSA and generates said PSA derived neoantigens at the tumor

In a preferred embodiment of the above method, PSA neoantigen generating compound is an irreversible enzyme inhibitor of PSA. In an even more preferred embodiment, the neoantigen generating inhibitor is comprised of an irreversible inhibitor of PSA and a targeting ligand that binds to PSMA, or Urokinase, or sigma receptors, or plasmin, or a matrix metalloproteinase.

A preferred embodiment of the present invention is a method of to treat A patient with prostate cancer comprised of the followng steps:

a) Sensitizing the patient against a set of neoantigens derived from multiple tumor-associated proteins that are enriched at prostate cancer cells; and

b) Administering, to the patient, a set of compounds that irreversibly modify said tumor-associated proteins thereby generating neoantigens;

In a preferred embodiment the set of administered compounds that generate the neoantigens irreversibly modify PSA and one or more of the proteins from the following list: Human glandular kallikrein 2, and Prostatic acid phosphatase, Plasmin, Urokinase, Fatty Acid Synthase, Epidermal growth factor receptors, Mitogen activated protein kinase kinase; Phosphatidylinositol 3-kinase, Thymidylate synthase, or Protein kinase A, or Fibroblast activation protein or seprase, or P-glycoprotein, or Ribonucleotide diphosphate reductase, or Dihydrofolate reductase, or Src Kinases, or Platelet-derived growth factor receptors, or MMP 7, or MMP 1, or MMP 2, or MMP 3, or MMP 9, or MMP 12, or MMP 13, or Membrane type MMP 1, or a Cathepsin, or Cathepsin B, or PSMA; In a preferred embodiment of the above, the neoantigen generating compounds are also comprised of one or more targeting lignads for one or more receptor that are increased at prostate tumor cells.

A preferred embodiment of the present invention is a method to treat a patient with breast cancer, or a patient with other forms of cancer, that have over-expression of the epidermal growth factor receptor, or related proteins which is comprised of:

a) Sensitizing the patient against a set of neoantigens derived from said epidermal growth factor receptor

b) Administering, to the patient, a compound that interacts with the epidermal growth factor receptor and generates said neoantigens at the tumor.

In a preferred embodiment the neoantigen generating compound is comprised of at least one targeting ligand that binds to a receptor that is increased on breast cancer cells. In a preferred embodiment, the neoantigen generating compound ET is comprised of two different tumor-selective targeting ligands. In a preferred embodiment, the effector group that irreversibly chemically modifies the epidermal growth factor receptor is a structure of embodiment Eneo 31, Eneo32, Eneo33, Eneo34, Eneo35. . . or Eneo42.

Methods of Drug Synthesis

The drugs of the present class can be prepared by a variety of synthetic approaches well known to one skilled in the arts. In order to effectively treat cancer, multiple targeted drugs can be required. Accordingly, a modular approach is preferred in which a small number of basic components such as linkers, triggers, and masked intracellular transporter ligands are synthesized and coupled with the desired targeting ligands and effector groups. A large variety of methods can be utilized to couple the respective components. The general steps include chemical protection of interfering groups, coupling, and deprotection. A preferred type of coupling reaction is the formation of an amide or ester bond. General references are given below and synthetic methodologies illustrated by examples that follow. The following references relate to this subject matter: Bodanszky M.; Bodanszky A. (1994) “The Practice of Peptide Synthesis” Springer-Verlag, Berlin Heidelberg; Greene, Theodora W.; Wuts, Peter G. M. (1991) “Protective Groups in Organic Synthesis” John Wiley & Sons, Inc.; March, Jerry (1985) “Advanced Organic Chemistry”, John Wiley & Sons Inc., the contents of which are incorporated herein by reference in their entirety.

The content of all references sited within this document are hereby incorporated by reference in entirety.

Equivalents

Those skilled in the arts can recognize or be able to ascertain, using no more then routine experimentation, many equivalents to the inventions, materials, methods, and components described herein. Such equivalents are intended to be within the scope of the claims of this patent.

While this invention has been particularly shown and described with references to preferred embodiments thereof, it will be understood by those skilled in the art that various changes in form and details may be made therein without departing from the scope of the invention encompassed by the appended claims.

### EXAMPLES

The following examples serve to illustrate certain aspects of the present invention. One skilled in the arts will recognize many other examples that are within the scope of the present invention. One skilled in the arts will recognize many instances where alternate reagents, protecting groups, or reaction sequences may be employed to prepare compounds encompassed by the present invention. The length of the various linker groups employed in the following examples can readily be changed by appropriate substitutions without altering the chemistry.

General Comments

In the following examples, the terms “coupled” or “coupling” are used to refer to the formation of an ester or amide bond from an alcohol or amine and acid. A large number of agents and methods are well known to one skilled in the arts for the coupling of amine or alcohols to acids. Relevant coupling agents and methods may be found within the following reference relates to this subject mafter: Bodanszky M.; Bodanszky A. (1994) “The Practice of Peptide Synthesis” Springer-Verlag, Berlin Heidelberg; Trost, Barry; (1991) Comprehensive Organic Synthesis, Pergamon Press, the contents of which are incorporated herein by reference in their entirety.

Unless otherwise specified, all reactions described in the examples can be conducted in an inert solvent under an inert atmosphere 4. All compounds and intermediates, unless indicated, can be purified by routine methods such as chromatography, distillation, or crystallization and stored in a stable form.

In compounds with chiral centers, the R, S, and racemic mixtures are to be considered within the scope of the present invention unless otherwise specified or unless specified in the references that relate to the starting materials or components.

Some of the normenclature employed in the following examples was generated by the software CS Chemdraw 5.0, CambridgeSoft Corporation.

Abbreviations:

Bsm-(1,1-Dioxo-1H-1λ6-benzo[b]thiophen-2-yl)-methyl

Bsmoc-(1,1-Dioxo-1H-1λ6-benzo[b]thiophen-2-yl)-methoxy-carbonyl

Fm-(9H-Fluoren-9-yl)-methyl

Fmoc-(9H-Fluoren-9-yl)-methoxy-carbonyl

TDBS-Tert-butyidimethylsilyl

### Example 1.1, 1.2

Prostatic adenocarcinoma cells have high concentrations of the enzyme Glutamate carboxypeptidase 11 or Prostatic Specific Membrane Antigen (PSMA) on the cell surface. PSMA is a zinc carboxypeptidase, which catalyzes the hydrolysis of N-acetyl-aspartylglutamate and gamma glutamates. The enzyme is potently inhibited by phosphorous based transition state analogs. 2-(phosphonomethyl)-pentanedioc acid inhibits the enzyme with a Ki of 0.3 nanomolar. The following references relate to this subject matter: U.S. Pat. No. 5,804,602 Sep. 8, 1998 Slusher, et al., “Methods of Cancer Treatment Using NAALADase Inhibitors”; U.S. Pat. No. 5,795,877 Aug. 18, 1998 Jackson, et al., “Inhibitors of NAALADase Enzyme Activity”; Jackson PF, et al., “Design, Synthesis, and Biological Activity of a Potent Inhibitor of the Neuropeptidase N-Acetylated Alpha-Linked Acidic Dipeptidase,” J Med Chem, 39(2):619-22 (1996), the contents of which are incorporated herein by reference in their entirety.

Compound 1 was synthesized and found to potently inhibit PSMA with a Ki of 20 nanomolar. Structure 1 has the fluorescent dye, Texas red, coupled by a linker

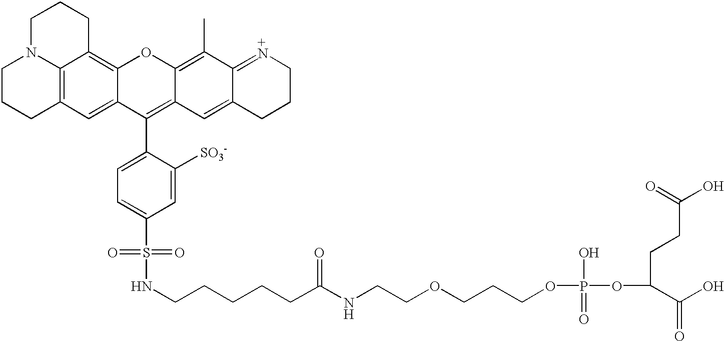

to a moiety that tightly binds to the active site of PSMA.

### Compound 1

Compound 1.1 was also synthesized and found to inhibit PSMA with a Ki of 3.4 nanomolar.

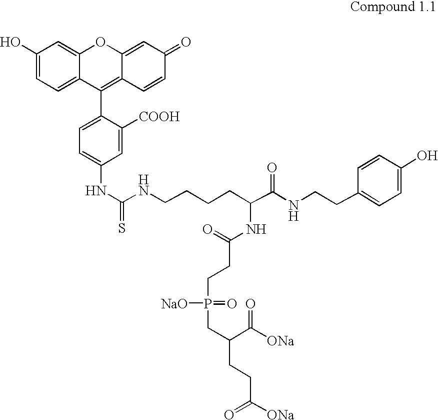

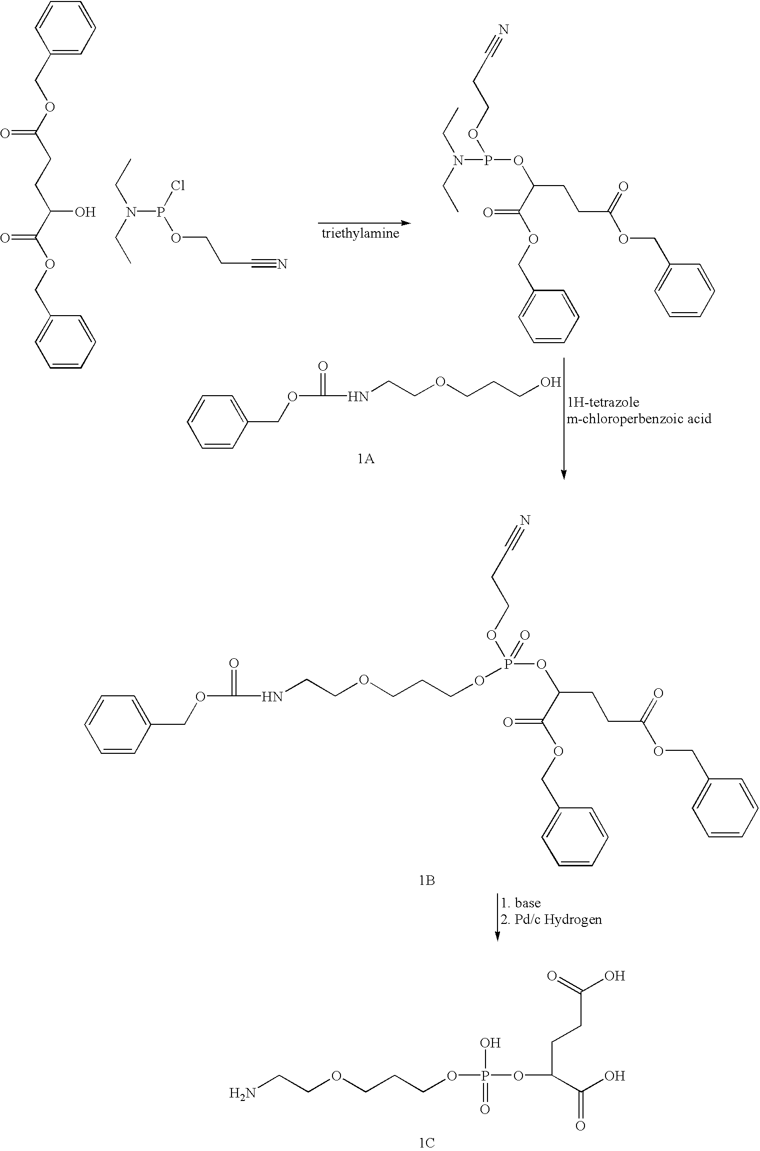

Compound 1 was synthesized by the scheme shown below:

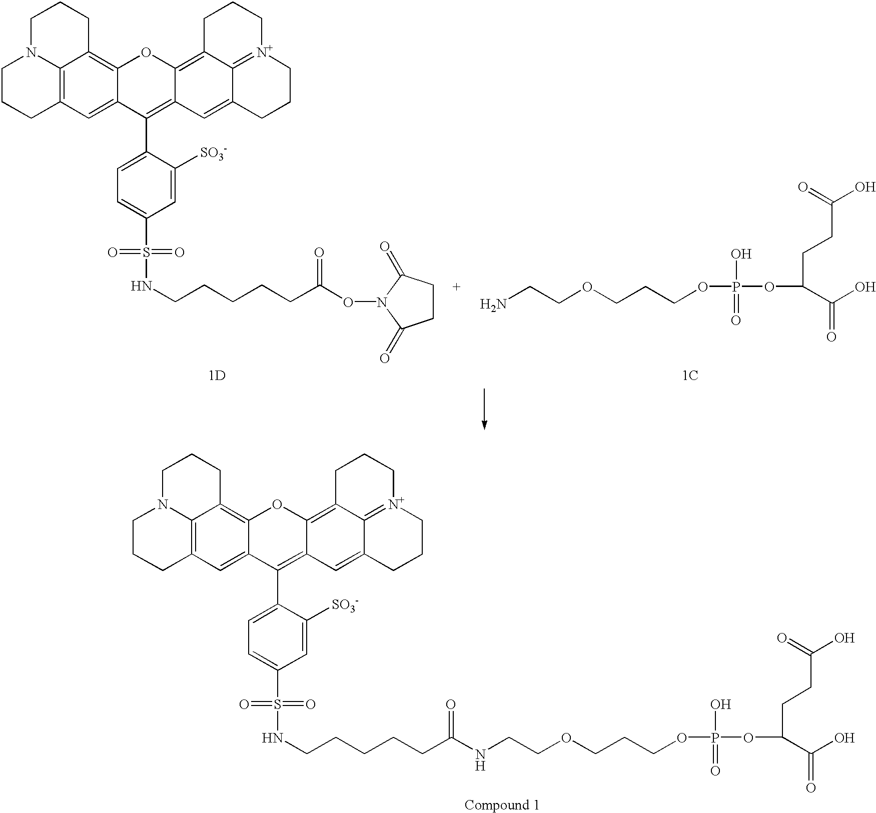

The Synthesis of Compound 1

Dibenzyl 2-hydroxyglutarate (5 mM) in 10 ml tetrahydrofuran and 5 mM of triethylamine was reacted at −78° C. with 2-cyanoethyl N,N diisopropyl chlorophosphoramidite. After 1 hour 5.3 mM of 1 A was added along with 5.3 mM of 1H tetrazole in 2 ml dimethylformamide. The reaction was allowed to warm to room temperature. After 2 hours it was cooled again to −78° C. and 5.5 mM of m-Cl-perbenzoic acid in 5 ml dimethylformamide was added. After 20 minutes the reaction was allowed to warm to room temperature. Compound 1B was then purified by silica gel chromatography using a gradient from 100% chloroform to 50:1 chloroform methanol. Yield was 74%. NMR was consistent with the structure 1B.

Compound 1B 135 mg was dissolved in 5 ml of acetonitrile and 2.4 ml of triethylamine was added. After 24 hours the solvent was evaporated and the residue dissolved. 4 ml methanol and 12 mg of 10% Pd on carbon was added. The suspension was then treated with hydrogen at atmospheric pressure for 4 hours, filtered and evaporated to yield 108 mg of 1 C. Proton and phosphorous NMR were consistent with structure 1C.

Compound 1 was synthesized by the reaction of the tributylammonium salt of 1 C (10 micromoles) and 3 micromoles of 1D (Molecular Probes) in 150 microliters of dimethylformamide and 10 microliters of tributylamine at room temperature for 12 hours. Compound 1 was then purified by preparative reverse phase HPLC using a C18 column and elution with a gradient from 0 to 70% acetonitrile in 20 mM ammonium bicarbonate buffer. The structure of compound 1 was confirmed by proton phosphorous and 2 dimensional proton NMR.

Compound 1.1 was synthesized according to Schemes 1-2.

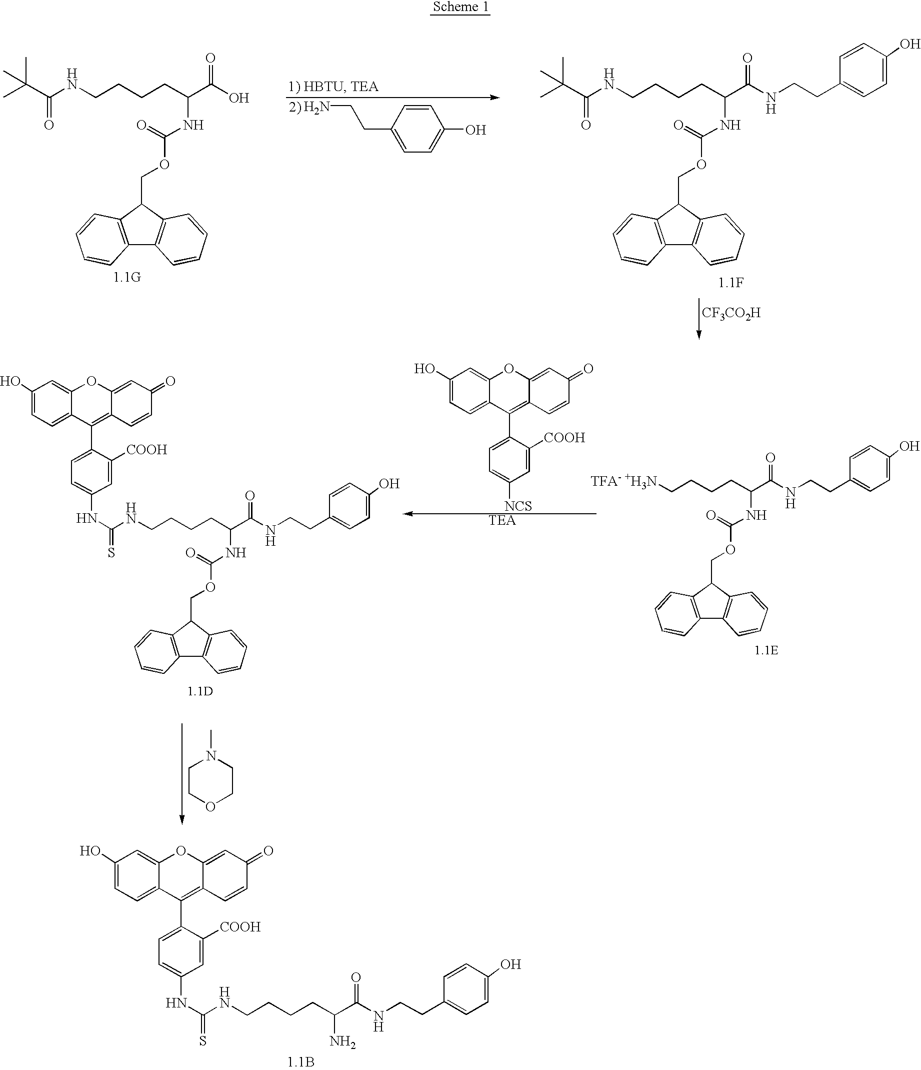

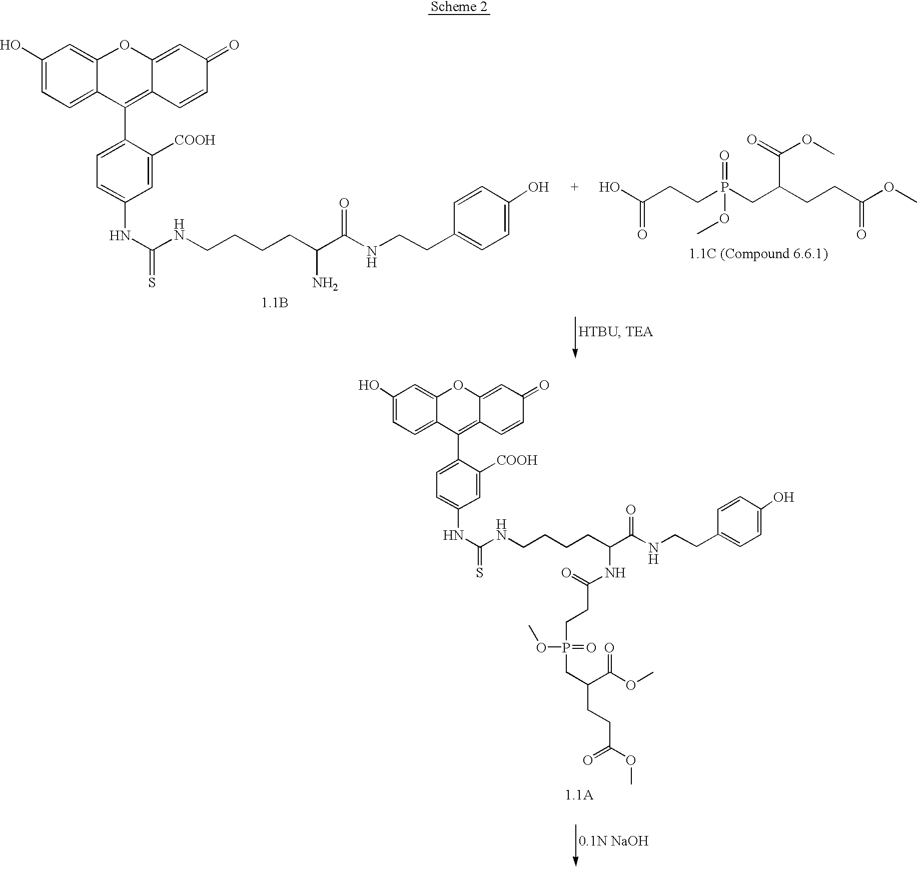

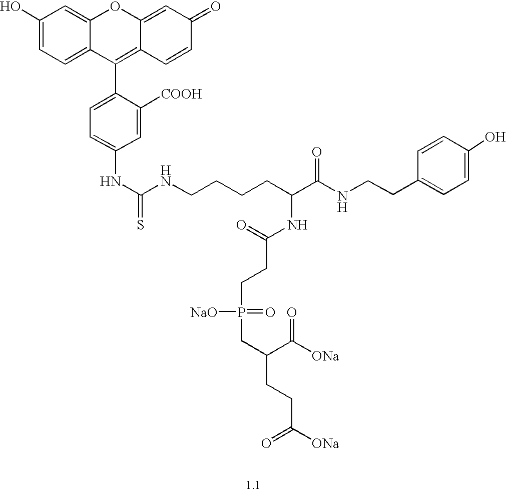

N^{α}-Fmoc-N^{ε}-Boc-L-lysine (10 mmol) in DMF (20 mL) was activated with HBTU (O-(1H-benzotriazole-1-yl)-N,N,N′,N′-tetramethyluronium hexafluorophosphate) and triethylamine (10 mmol of each) for 5 min., and condensed with 10 mmol of 4-(2-aminoethyl)phenol for 6 hours at r.t.(room temperature). The reaction mixture was evaporated under vacuum, dissolved in ethylacetate, washed with 0.5 M citric acid, water, 5% NaHCO₃, water, brine, and evaporated under vacuum. Flash chromatography on silicagel (chloroform/methanol 50:1) afforded 7.0 mmol, (70% yield) of compound 1.1F, which was characterized by ¹H NMR.

Compound 1.1F (1 mmol) was dissolved in a 50% solution of trifluoroacetic acid in chloroform. The solution, after 1 hour at r.t., was evaporated under vacuum, re-evaporated from toluene, and dried under vacuum to give compound 1.1E as a glassy residue (¹H NMR). This residue was dissolved in chloroform (3 mL) and treated with a solution of fluorescein-5-isothiocianate (1 mmol) in 1 mL DMF and 0.3 mL triethylamine. After 3 hours at r.t., the mixture was evaporated under vacuum. Flash chromatography of the residue (gradient from 10% to 20% methanol in methylene chloride) afforded 0.63 mmol (yield, 63%) of compound 1.1 D, the structure of which was confirmed by ¹H and COSY NMR.

Compound 1.1D (0.114 mmol) was dissolved in a mixture of 250 microliters of DMF and 250 microliters of N-methylmorpholine. The solution was kept tightly closed for 24 hours at 45° C., and then evaporated under vacuum. The residue was extracted two times with hexane and dried under vacuum to give quantitative yield of compound 1.1B.

(Compound 1.1C is the same as compound 6.6.1, the synthesis of which is described at a later point).

Compound 1.1C (0.144 mmol) was activated with HBTU and N-methylmorpholine (0.158 mmol of each) in 0.350 mL of DMF for 5 min. at r.t. and the resulting solution was added to the solution of compound 1.1B (0.114 mmol) in 0.300 mL of DMF. After 24 hours at r.t., the reaction mixture was evaporated under vacuum and compound 1.1A was isolated by flash chromatography on silicagel (gradient from chloroform/methanol 10:1 to chloroform/methanol/water 50:10:1) in 51% yield. The product was characterized by ¹H, COSY, and ³¹P NMR.

Compound 1.1A (27 micromols) was de-blocked to compound 1.1 by treatment with methanol/0.1 N NaOH 1:1 for 2.5 hours. Compound 1.1 was isolated by preparative reverse phase HPLC (20% acetonitrile in 20 mM ammonium bicarbonate buffer pH 7.8) in 52% yield as the ammonium salt. The structure was confirmed by ¹H, COSY, and ³¹P NMR.

Inhibition of PSMA by Compounds 1 and 1.1

PSMA was prepared from LNCaP prostatic carcinoma cells as described by Pinto J. T. et al., Clinical Cancer Res. Vol.2 p.1445 (1996). The enzyme was assayed employing radiolabelled N-acetylaspartylglutamate as a substrate and monitoring the generation of glutamic acid in the presence of varying concentrations of Compound 1 or 1.1. The data demonstrated that Compound 1 inhibited the enzyme 50% at a concentration of 20 nM. Compound 1.1 inhibited the enzyme 50% at 3.4 nM.

### Example 2

Compound 2 is a multifunctional drug delivery vehicle with targeting ligands for urokinase, matrix metalloproteinases (1,2,3,9, and MT-MMP-1) and melanocyte stimulating hormone receptor. The drug has a masked folic acid group as an intracellular transport ligand that will be activated by esterase. Bleomycin A2 will be freed upon cleavage of a disulfide trigger by thiol reductases. Five hundred molecules of bleomycin delivered intracellularly are sufficient to kill a cell. The drug is expected to have activity against malignant melanoma. The following references relate to this subject matter: Pron G., et al., “Internalisation of the Bleomycin Molecules Responsible for Bleomycin Toxicity: A Receptor-mediated Endocytosis Mechanism,” Biochemical Pharmacology, 57:45-56 (1999), the contents of which are incorporated herein by reference in their entirety.

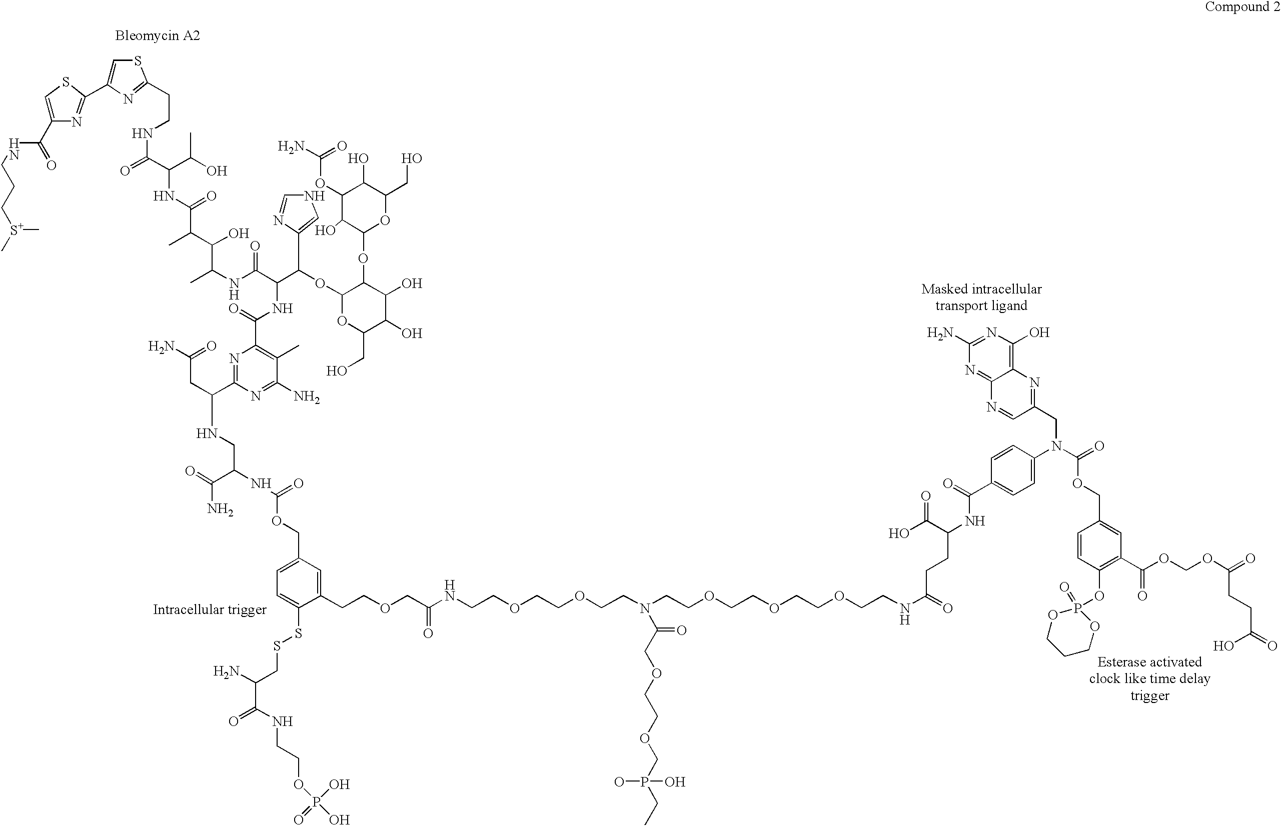

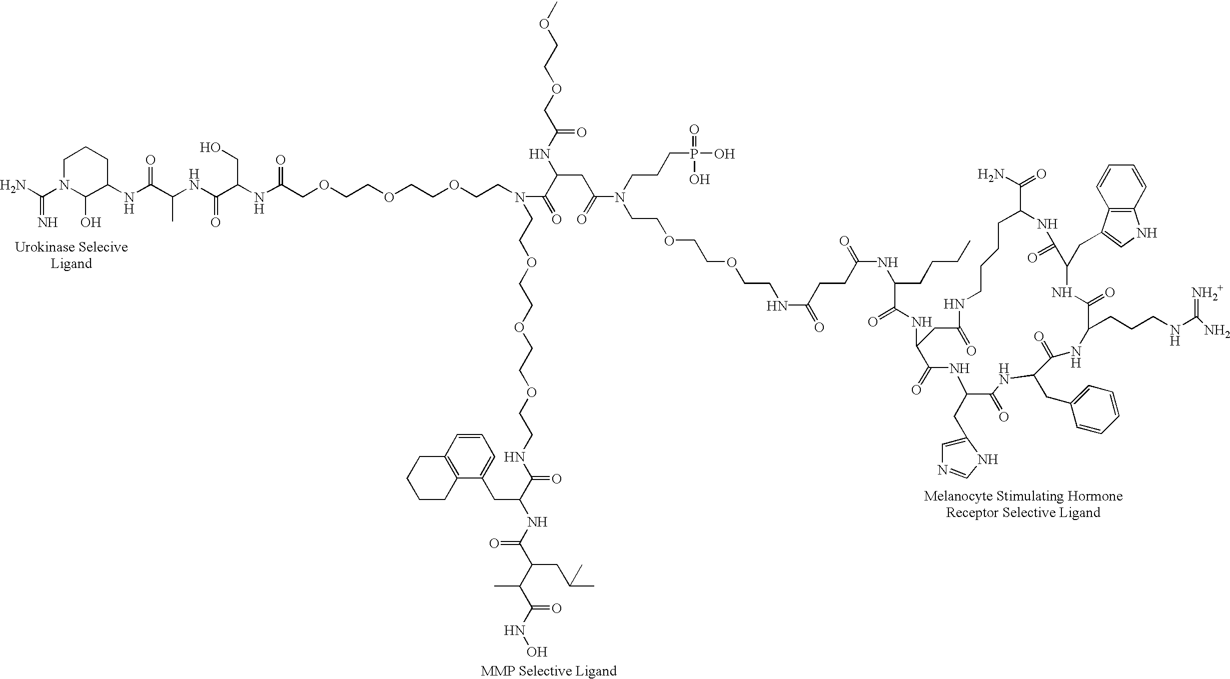

Compound 2 may be prepared by the methods similar to those described for compound 24 by replacing compound 23.2.a with bleomycin A2.

### Example 3

Compound 3 is a multifunctional drug delivery vehicle that will be selective for prostatic cancer cells that bear both the laminin receptor and PSMA. The drug has a masked folic acid moiety, as an intracellular transport with a clock like time delayed trigger that will be activated by esterase. The toxin Ecteinascidin 743 will be liberated following activation of the intracellular trigger by intracellular glutathione or by thioreductases. Ecteinascidin 743 is cytotoxic at picomolar concentrations. The following references relate to this subject matter: Zewail-Foote M.; Hurley L. H., “Ecteinascidin 743: A Minor Groove Alkylator that Bends DNA toward the Major Groove,” J Med Chem, 42(14):2493-2497 (1999); Takebayashi Y., et al., “Poisoning of Human DNA Topoisomerase I by Ecteinascidin 743, an Anticancer Drug that Selectively Alkylates DNA in the Minor Groove,” Proc Natl Acad Sci USA, 96:7196-7201 (1999); Hendriks H. R., et al., “High Antitumour Activity of ET743 against Human Tumour Xenografts from Melanoma, Non-Small-Cell Lung and Ovarian Cancer.” Ann Oncol, 10(10):1233-40 (1999), the contents of which are incorporated herein by reference in their entirety.

Compound 3 may be prepared by the methods described for compound 6 by replacing compound 6.2.0c with compound 3.1. Compound 3.1 may be prepared by reacting Ecteinascidin 743 with compound 3.2 in an inert solvent in the presence of a base such as pyridine and then selectively cleaving the Bsm ester with tris(2-aminoethyl)amine. Compound 3.2 may be prepared by treating compound 38.3 with phosgene in an inert solvent.

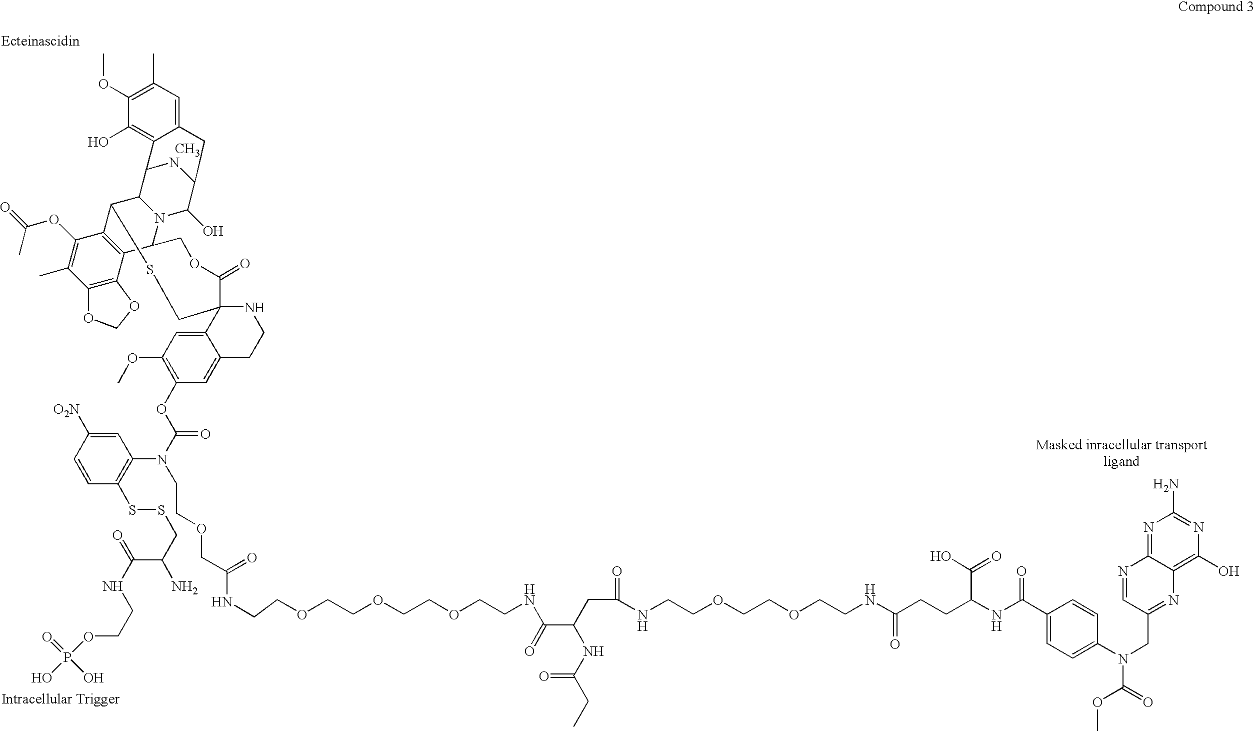

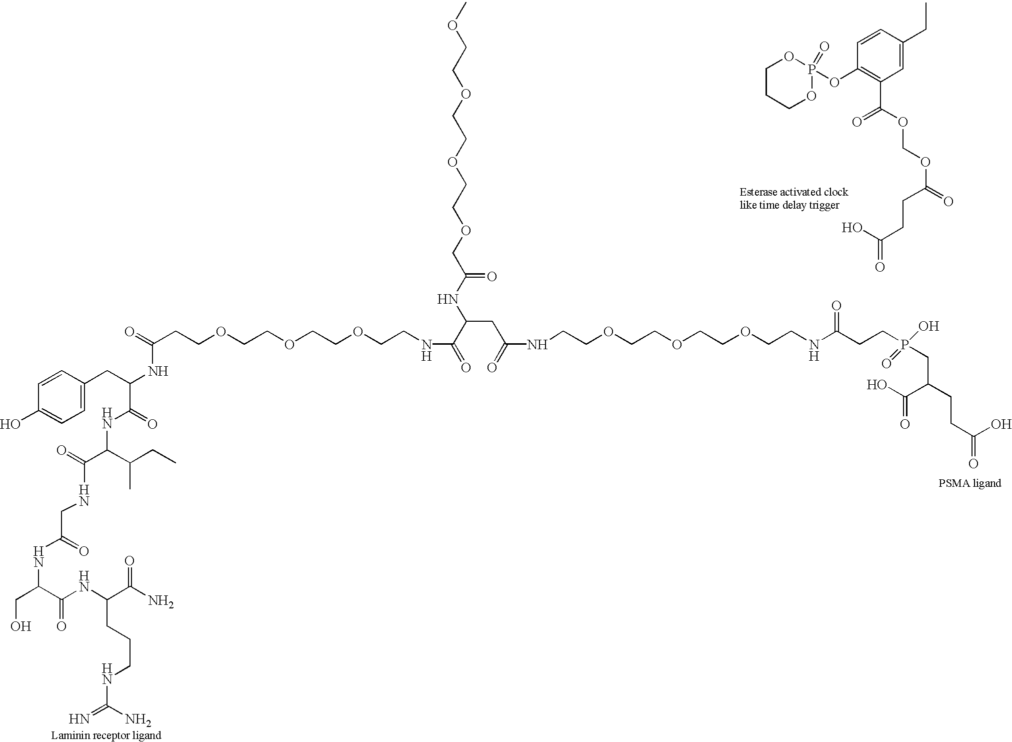

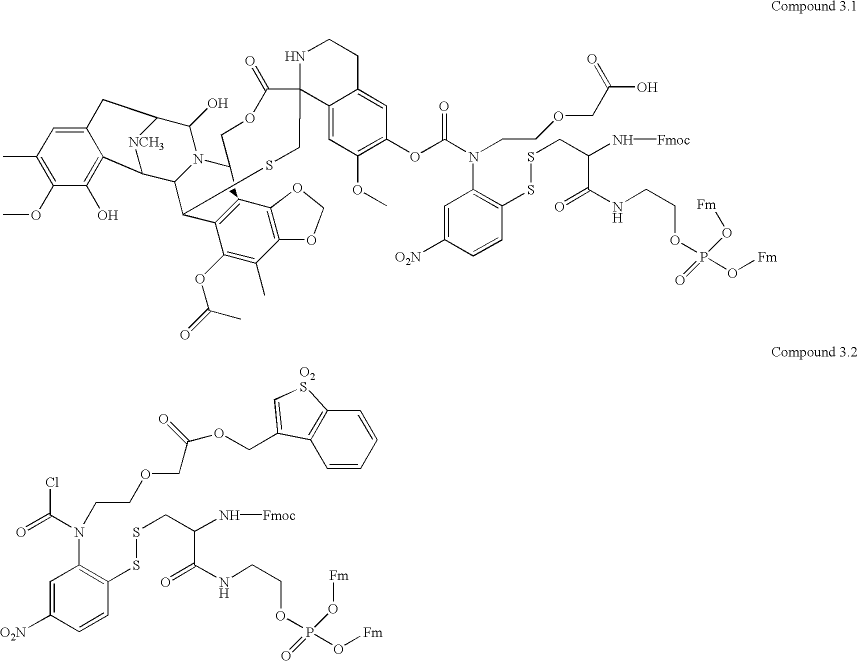

### Example 4

Compound 4 is a multifunctional drug delivery vehicle with targeting ligands for urokinase and laminin receptors. Like compound 3, the drug will liberate Ecteinascidin 743 following activation of the intracellular trigger by glutathione or by thioreductases.

Compound 4 may be prepared by the methods described for compound 11 by replacing compound 6.2.Oc with compound 3.1.

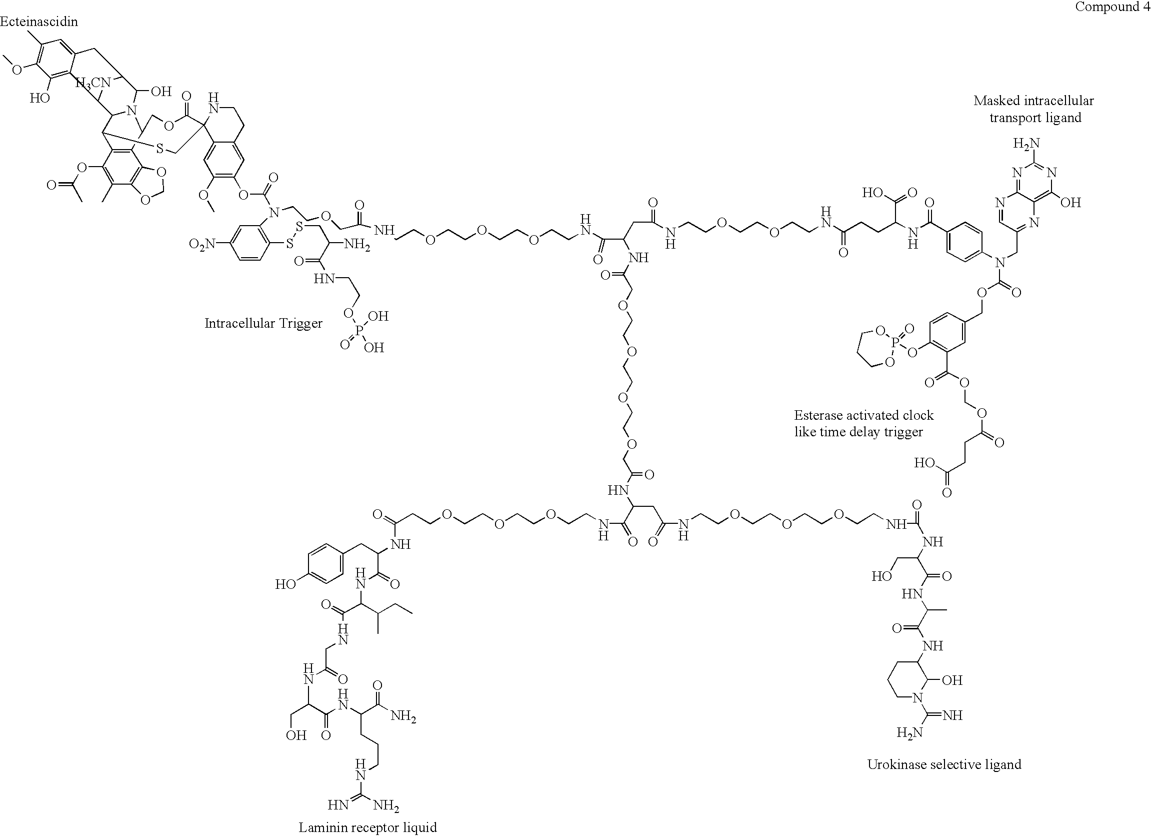

### Example 5

Compound 5 is a multifunctional drug delivery vehicle with targeting ligands for sigma receptors and MMP7, MMP2, MMP1, and MMP3. Didemnin B will be released upon activation of a trigger by plasmin. Didemnin B is cytotoxic at nanomolar to sub-nanomolar concentrations. However, Didemnin B has not proven clinically useful due to its poor antitumor selectivity and toxicity. Compound 5 will bind essentially irreversibly to the surface cells that are jointly positive for sigma receptors and the targeted matrix metalloproteinases. Tumor associated plasmin will toxify the drug by liberating the Didemnin B. Ubiquitous nonspecific esterases will detoxify the drug by opening the cyclic ring of the toxin. The ratio between plasmin mediated toxification and esterase mediated detoxification will determine the cytotoxicity. The drug is expected to have selective toxicity for cells that are jointly positive for sigma receptors, the targeted MMP's, and plasmin. The following references relate to this subject matter: Kiss I., et al., “Investigation on the Substrate Specificity of Human Plasmin using Tripeptidyl-P-Nitroanilide Substrates,” Biochem Biophys Res Comm, 131 (2):928-934 (1985); Sakai R., et al., “Structure-Activity Relationships of the Didemnins,” J Med Chem, 39:2819-2834 (1996); Meng L., et al., “The Antiproliferative Agent Didemnin B Uncompetitively Inhibits Palmitoyl Protein Thioesterase,” Biochemistry, 27:10488-10492 (1998); Ahuja D., et al., “Inhibition of Protein Synthesis by Didemnin B: How EF-1α: Mediates Inhibition of Translocation,” Biochemistry, 39:4339-4346 (2000); Mittelman A., et al., “Phase II Clinical Trial of Didemnin B in Patients with Recurrent or Refractory Anaplastic Astrocytoma or Glioblastoma Multiforme (NSC 325319),” Invest New Drugs, 17(2):179-82 (1999); Jones D. V., et al., “Phase II Study of Didemnin B in Advanced Colorectal Cancer,” Ivest New Drugs, 10(3):211-3 (1992); Grubb D. R., et al., “Didemnin B Induces Cell Death by Apoptosis: The Fastest Induction of Apoptosis ever Described,” Biochem Biophys Res Commun, 215(3):1130-6 (1995); Kucuk O., et al., “Phase II Trial of Didemnin B in Previously Treated Non-Hodgkin's Lymphoma: An Eastern Cooperative Oncology Group (ECOG) Study,” Am J Clin Oncol, 23(3):273-7 (2000); Sondak V. K., et al., “Didemnin B in Metastatic Malignant Melanoma: A Phase II Trial of the Southwest Oncology Group,” Anticancer Drugs, 5(2):147-50 (1994); Williamson S. K., et al., “Phase II Evaluation of Didemnin B in Hormonally Refractory Metastatic Prostate Cancer. A Southwest Oncology Group Study,” Invest New Drugs, 13(2):167-70 (1995); Lobo C., et al., “Effect of Dehydrodidemnin B on Human Colon Carcinoma Cell Lines,” Anticancer Res, 17(1 A):333-6 (1997); Geldof A. A., et al., “Cytotoxicity and Neurocytotoxicity of New Marine Anticancer Agents Evaluated using in Vitro Assays,” Cancer Chemother Pharmacol, 44(4):312-8 (1999), the contents of which are incorporated herein by reference in their entirety.

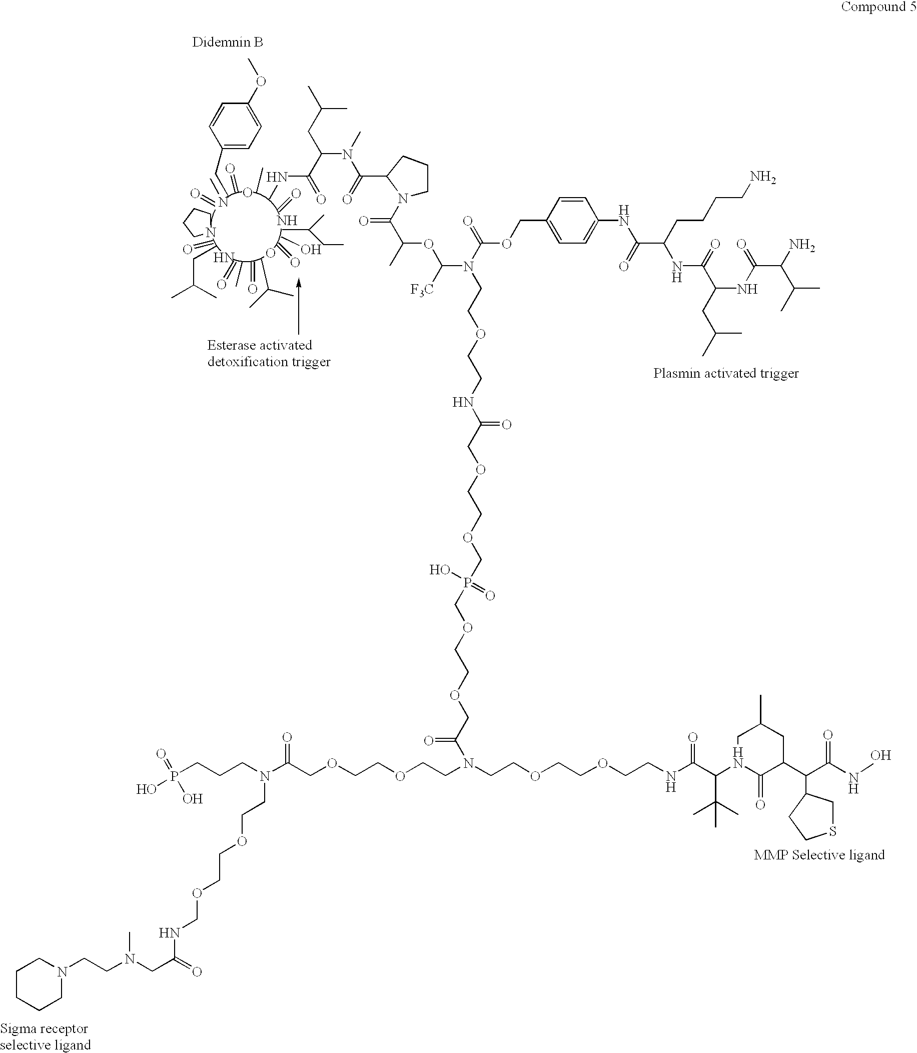

Compound 5 may be prepared by a multi-step process. Compound 5.1 may be coupled to compound 1 7.4b. The product may then be treated with Zn and acid to remove the trichloroethoxycarbonyl group. The product may then be coupled to compound 20.2.2a. The Bsm ester may then be selectively cleaved with tris(2-aminoethyl)amine. The product may then be coupled with compound 5.2. Deprotection with acid to remove the pixyl group followed by treatment with base to remove the Fm and Fmoc groups will give compound 5.

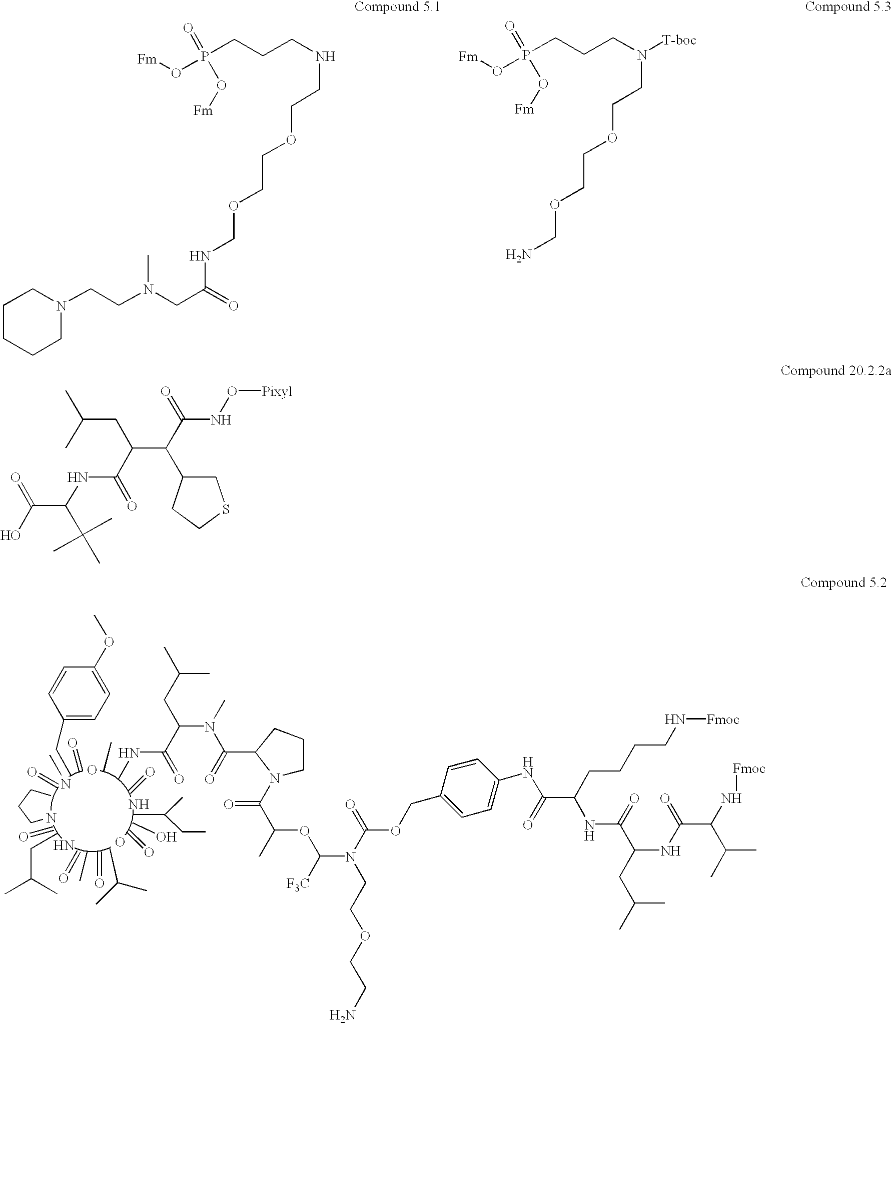

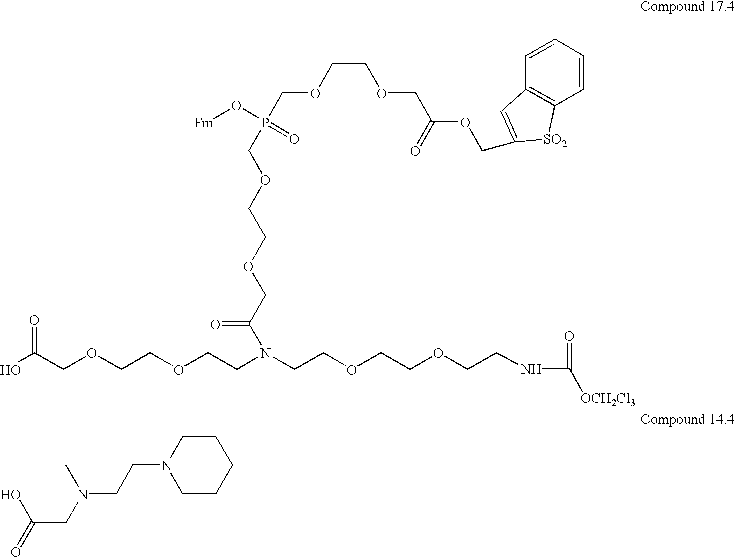

Compound 5.1 may be prepared by coupling compound 14.5 and compound 5.3 and then treating with acid to remove the T-Boc group. Compound 5.3 may be prepared by treating compound 1 3b3 with di-t-butyl pyrocarbonate in an inert solvent and then selectively removing the trityl group with acid.

Compound 5.2 may be prepared by reacting Didemnin B and compound 5.4 in an inert solvent in the presence of base and then selectively removing the Bsmoc group with tris(2-aminoethyl)amine.

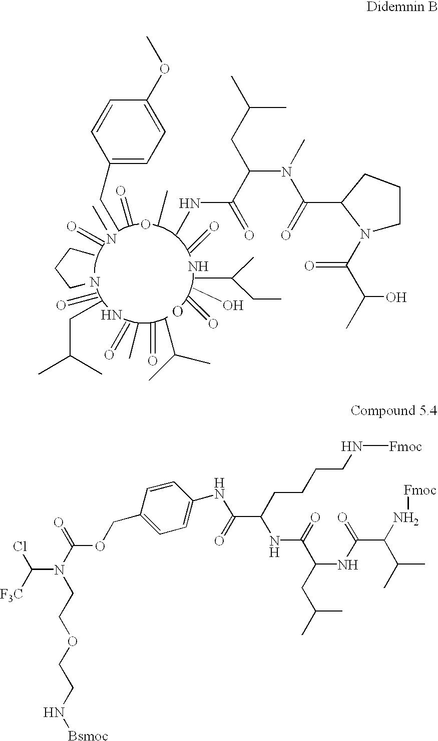

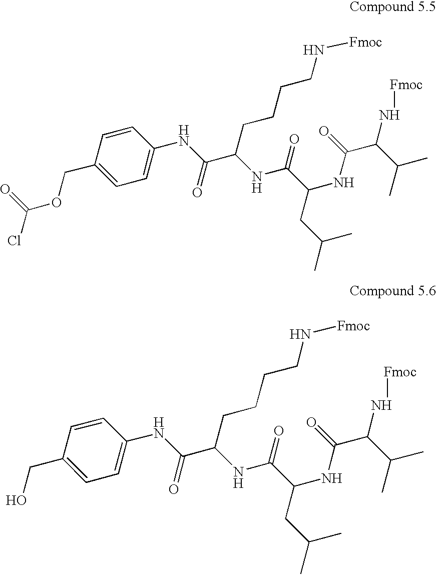

Compound 5.4 may be prepared by a multistep process. Compound 5.5 may be reacted with [2-(2-Amino-ethoxy)-ethyl]-carbamic acid 1,1-dioxo-1 H-1λ6-benzo[b]thiophen-2-ylmethyl ester in an inert solvent in the presence of a base such as pyridine. The product may then be treated with trifluoroacetaldehyde in an inert solvent and then treated with a reagent such as phosphorous trichloride to give compound 5.4

Compound 5.5 may be prepared by treating compound 5.6 with phosgene in an inert solvent. Compound 5.6 may be prepared by a multi-step process. The compounds p-aminobenzyl alcohol and L-N-α-allyloxycarbonyl-N ε-Fmoc-lysine may be coupled and then the product may be deblocked by with Pd (0).

The product may then be coupled to L-N-allyloxycarbonyl leucine. Deblocking with Pd (0) followed by coupling to D-Fmoc-valine will give compound 5.6.

### Example 6

Compound 6 is a multifunctional drug delivery vehicle that will be selective for prostatic cancer cells that bear both the laminin receptor and PSMA. The drug has a masked folic acid moiety as an intracellular transport with a clock like time delayed trigger that will be activated by esterase. The toxin indanocine will be liberated following activation of the intracellular trigger by intracellular glutathione or by thioreductases. The affinity of the drug for PSMA+, laminin receptor+ cells should be extremely high as each ligand independently will bind with Ki in the nanomolar range. The following references relate to this subject matter: Leioni L., et al., “Indanocine, a Microtubule-Binding Indanone and a Selective Inducer of Apoptosis in Multidrug-Resistant Cancer Cells,” J Nat Cancer Inst, 92(3):217-224 (2000), the contents of which are incorporated herein by reference in their entirety.

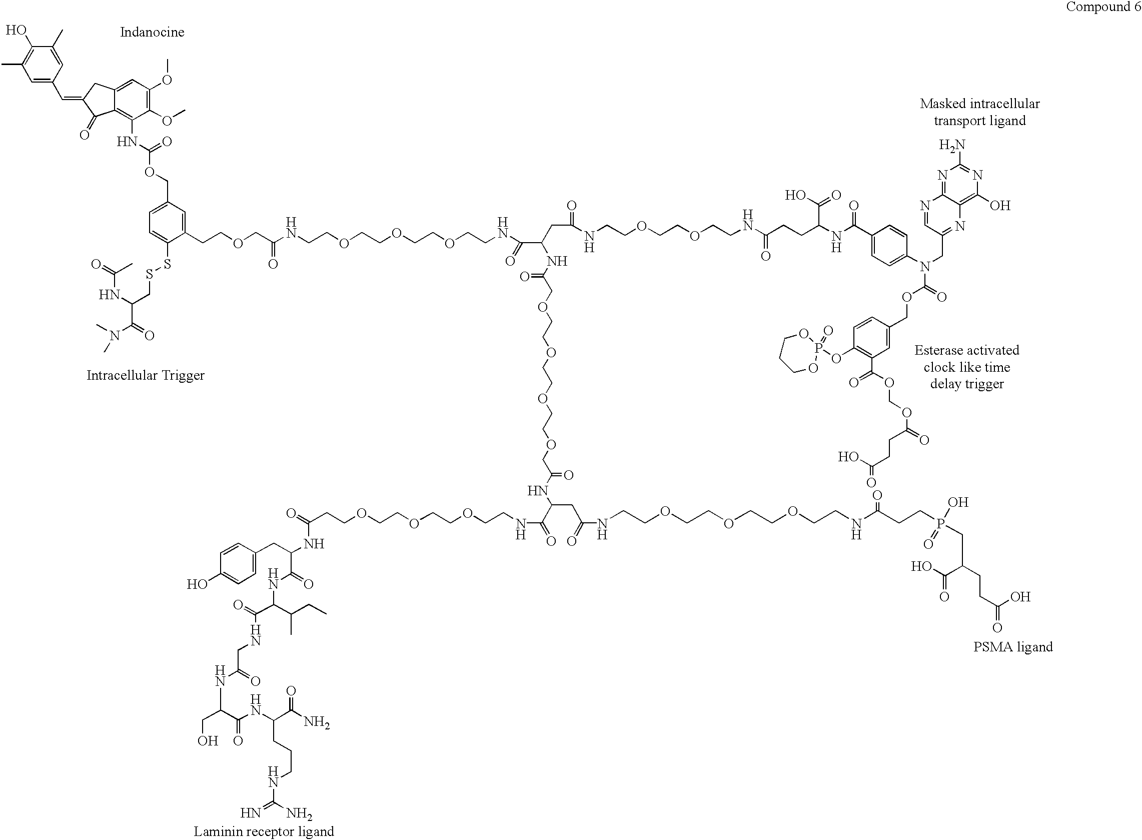

Compound 6 may be prepared by treating compound 6.1 with acid to remove the 2-Biphenyl-4-yl-propan-2-oxy-carbonyl protecting group and then treating with base to cleave the Fm esters.

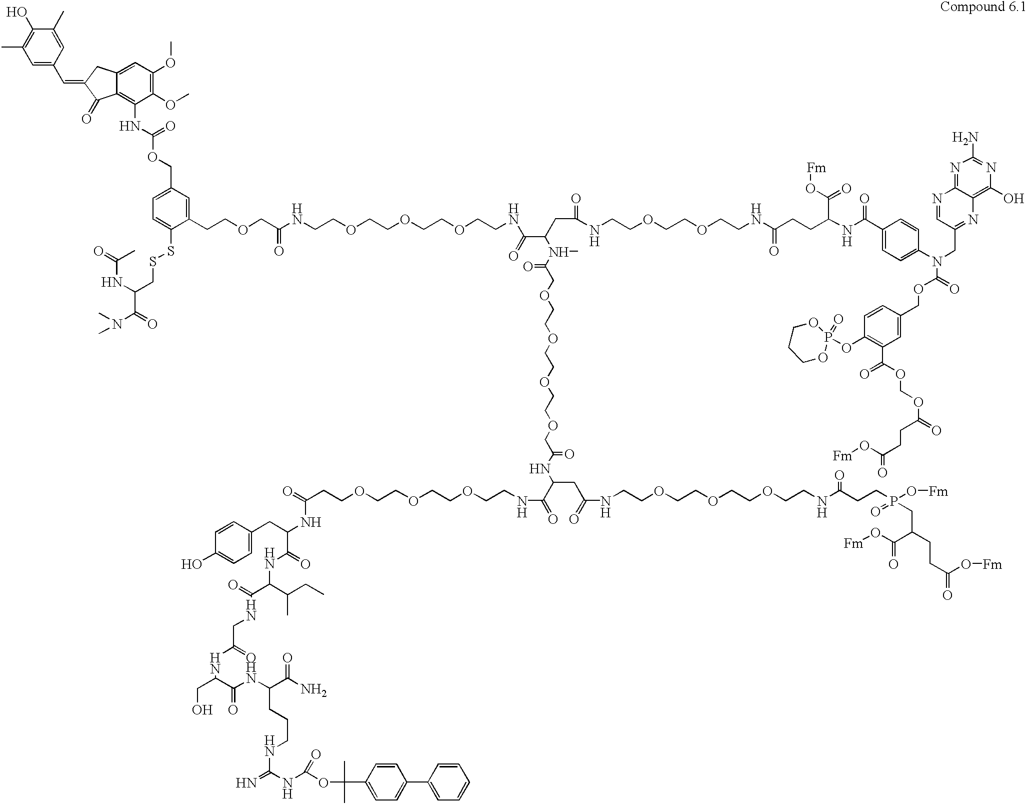

Compound 6.1 may be synthesized by coupling compound 6.2.0 and compound 6.2.1. Standard peptide coupling reagents, such as dicylohexycarbodimide or O-benzotriazol-1 -y-tetramethyluronium hexafluorophosphate, may be employed in an inert solvent with a base such as triethylamine.

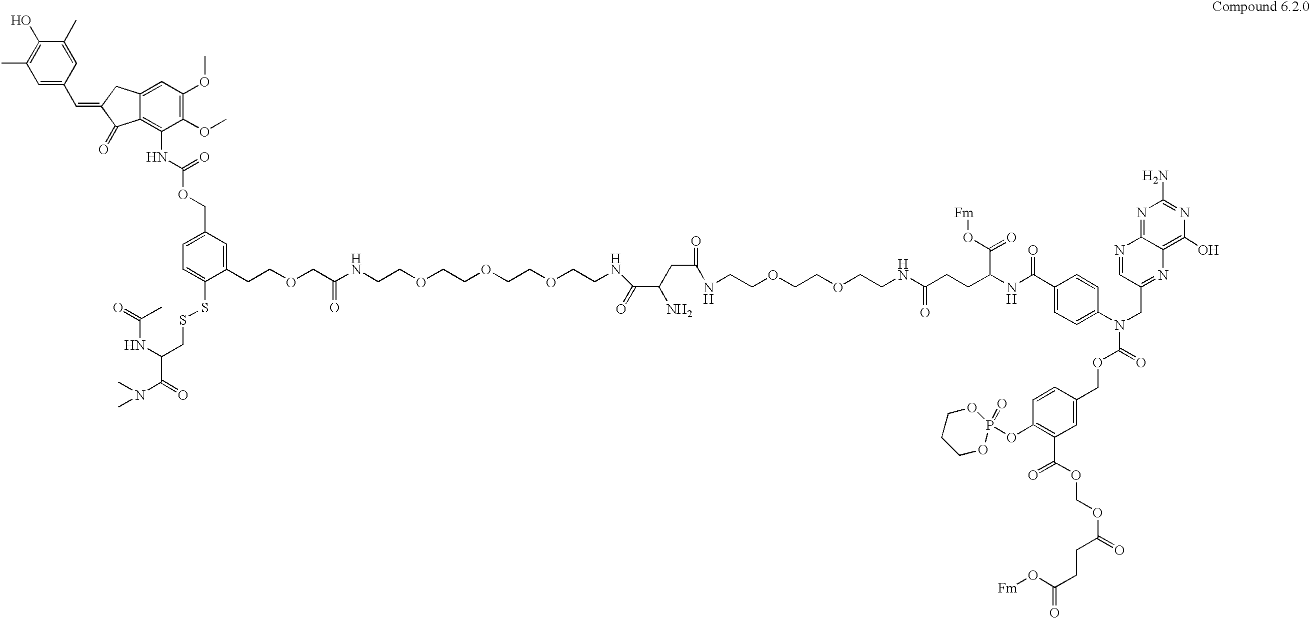

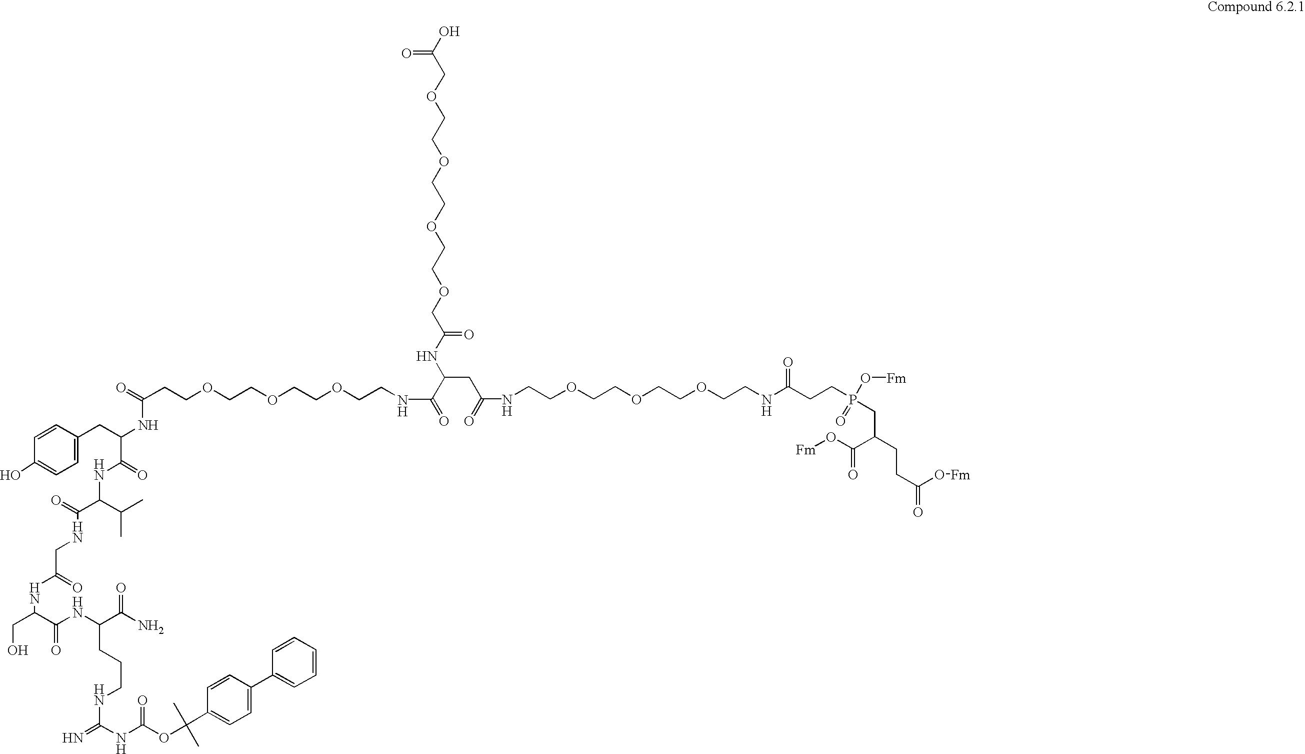

Compound 6.2.0 may be prepared by a multi-step process. Compound 6.2.Oa and compound 6.2.Ob may be coupled and then treated with Zn to remove the trichloroethoxycarbonyl group. The product may then be coupled with compound 6.2.0c. The Bsmoc group may then be selectively removed by treating with tris(2-aminoethyl)amine under conditions that will leave the Fmoc group intact to give compound 6.2.0. The following references relate to this subject matter: Just G.; Grozinger K., “A Selective, Mild Cleavage of Trichloroethyl Esters, Carbamates, and Carbonates to Carboxylic Acids, Amines, and Phenols using Zinc/Tetrahydrofuran/pH 4.2-7.2 Buffer,” Synthesis, 457-458 (1976); Carpino L. A., et al., “New Family of Base- and Nucleophile-Sensitive Amino-Protecting Groups. A Michael-Acceptor-Based Deblocking Process. Practical Utilization of the 1 ,1-Dioxobenzo[b]thiophene-2-ylmethyloxycarbonyl (Bsmoc) Group,” J Am Chem Soc, 119:9915-9916 (1997); Carpino L. A., et al., “The 1,1-Dioxobenzo[b]thiophene-2-ylmethyloxycarbonyl (Bsmoc) Amino-Protecting Group,” J Org Chem, 64:4324-4338 (1999), the contents of which are incorporated herein by reference in their entirety.

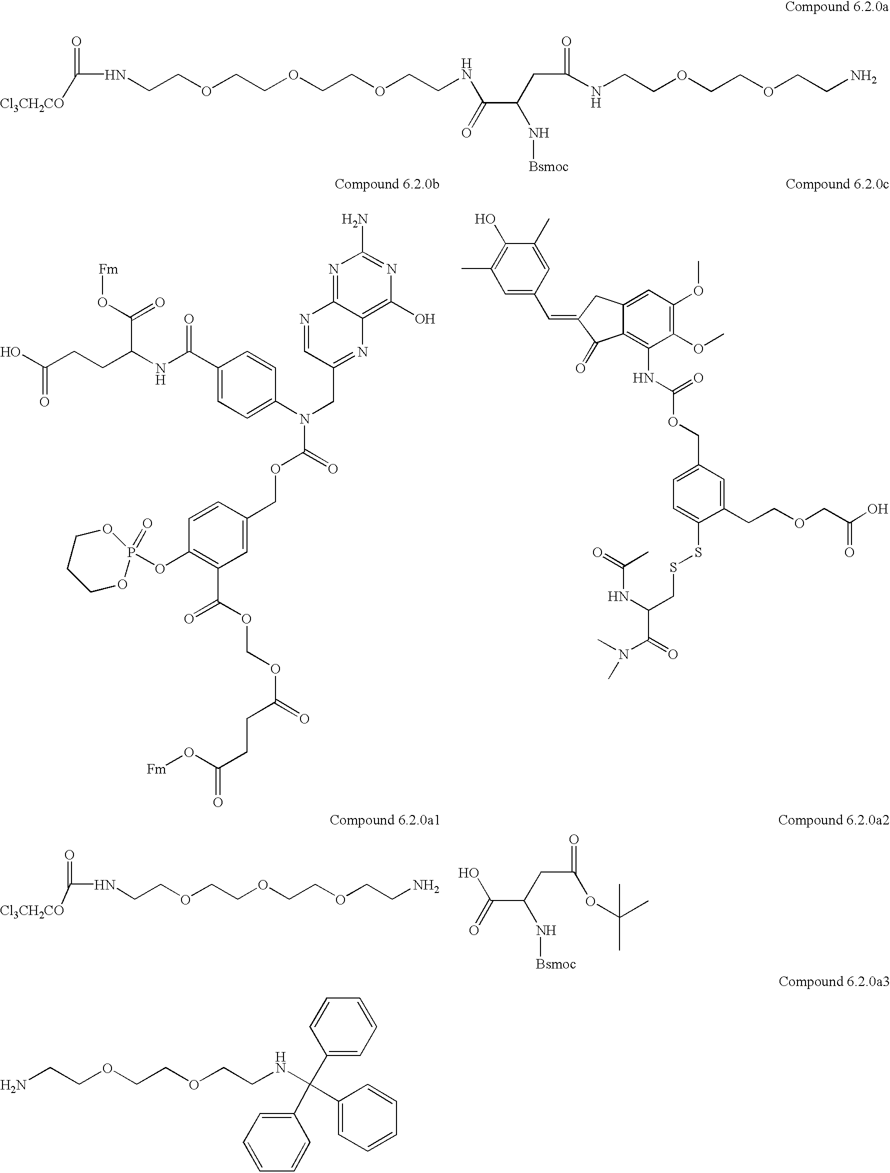

Compound 6.2.0a may be prepared by a multi-step process. Compound 6.2.0a1 may be coupled to compound 6.2.0a2. The product may then be treated with acid to cleave the t-butyl ester and then may be coupled to compound 6.2.0a3. Treatment with acid will then give compound 6.2.0a.

Compound 6.2.0al may be prepared by a multi-step process. Treating 2-{2-[2-(2-Amino-ethoxy)-ethoxy]-ethoxy}-ethylamine with one equivalent of trityl chloride and base and isolating the monotritylated product will give (2-{2-[2-(2-Amino-ethoxy)-ethoxy]-ethoxy}-ethyl)-trityl-amine. This may then be treated with 2,2,2 trichloroethyl chloroformate and base. Treatment with acid will then give compound 6.2.0a1.

Compound 6.2.0a2 may be prepared by treating L-aspartic acid β-t-butyl ester with (1,1 -Dioxo-1H-1λ6-benzo[b]thiophen-2-yl)-methyl chloroformate and base in an inert solvent or under Schotten-Bauman conditions.

Compound 6.2.0.a3 may be prepared by treating 2-[2-(2-Amino-ethoxy)-ethoxy]-ethylamine with one equivalent of trityl chloride and base.

Compound 6.2.0c may be prepared by reacting indanocine and compound 6.2.0b1 in an inert solvent in the presence of a base such as pyridine and then treating with with tris(2-aminoethyl)amine under conditions that will leave the Fm groups intact.

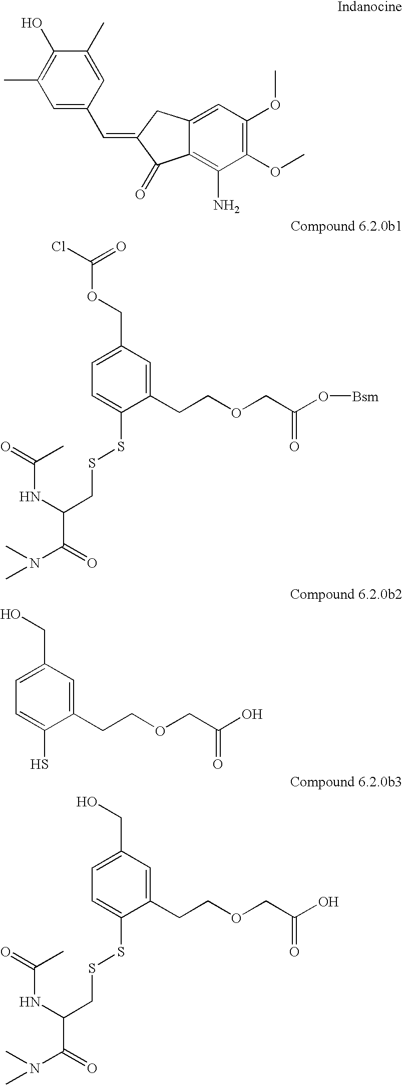

Compound 6.2.0b1 may be prepared by a multi-step process. Treating N-acetyl-L-cysteine N,N dimethylamide with diethyl azidocarboxylate, then reacting the product with compound 6.2.0b2, will give the mixed disulfide compound 6.2.0b3. Treatment with trityl chloride and base will give compound 6.2.0b4. Treatment with (1,1-Dioxo-1H-1λ6-benzo[b]thiophen-2-yl)-methanol and a reagent such as dicyclohexylcarbodiimide, followed by treatment with acid to remove the trityl group will give compound 6.2.0b5. Treatment with phosgene in an inert solvent will give compound 6.2.0b1.

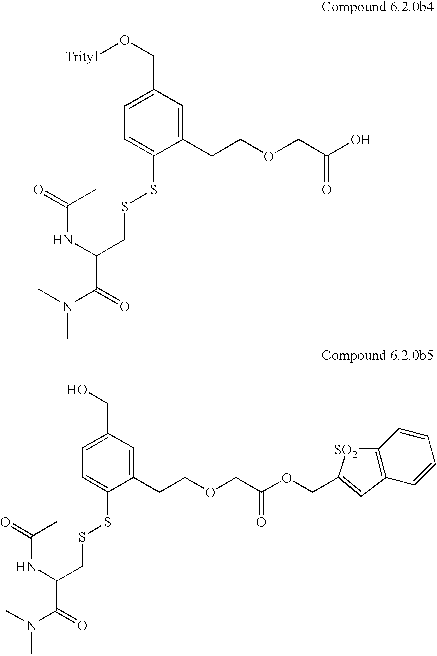

The following references relate to this subject matter: Mukaiyama T.; Takahashi K., “A Convenient Method for the Preparation of Unsymmetrical Disulfides by the use of Diethyl Azodicarboxylate,” Tetrahedron Letters, 56:5907-5908 (1968), the contents of which are incorporated herein by reference in their entirety.

Alternatively, a variety of other methods may also be employed to form the mixed disulfide compound described above. The following references relate to this subject matter: Harpp D. N., et al., “A New Synthesis of Unsymmetrical Disulfides,” Tetrahedron Letters, 41:3551-3554 (1970); Derbesy G.; Harpp D. N., “A Simple Method to Prepare Unsymmetrical Di- Tri- and Tetrasulfides,” Tetrahedron Letters, 35(30):5381-5384 (1994); Harpp D. N.; Back T. G., “The Synthesis of Some New Cysteine-Containing Unsymmetrical Disulfides,” J Org Chem, 36(24):3828-3829 (1971), the contents of which are incorporated herein by reference in their entirety.

Compound 6.2.0b2 may be prepared by a multi-step process. A Friedel-Crafts reaction between 4-mercapto-benzoic acid and chlorocarbonylmethoxy-acetic acid methyl ester will give 4-mercapto-3-(2-meethoxycarbonylmethoxy-acetyl)benzoic acid. Reduction of the resulting ketone with Zn/HCL will give 4-mercapto-3-(2-methoxycarbonylmethoxy-ethyl)-benzoic acid. Treatment with borane in a solvent such as tetrahydrofuran will reduce the carboxylic acid to the alcohol and give [2-(5-Hydroxymethyl-2-mercapto-phenyl)-ethoxy]-acetic acid methyl ester. Hydrolysis of the methyl ester will give compound 6.2.0b2. The following references relate to this subject matter: Gore P. H., “Aromatic Ketone Synthesis,” in Friedel-Crafts and Related Reactions, Olah G. A. (edt.), John Wiley & Sons, p.55 (1964); Read R. R.; Wood J. Jr., “o-n-Heptylphenol,” Org Syn Coll Volume 3, pp.444-446; Yoon N. M.; Pak C. S., “Selective Reductions. XIX. The Rapid Reaction of Carboxylic Acids with Borane-Tetrahydrofuran. A Remarkable Convenient Procedure for the Selective Conversion of Carboxylic Acids to the Corresponding Alcohols in the Presence of Other Functional Groups,” J Org Chem, 33(16):2786-2792 (1973), the contents of which are incorporated herein by reference in their entirety.

Compound 6.2.0b may be described by methods detailed in example 32. (See compound 32.1).

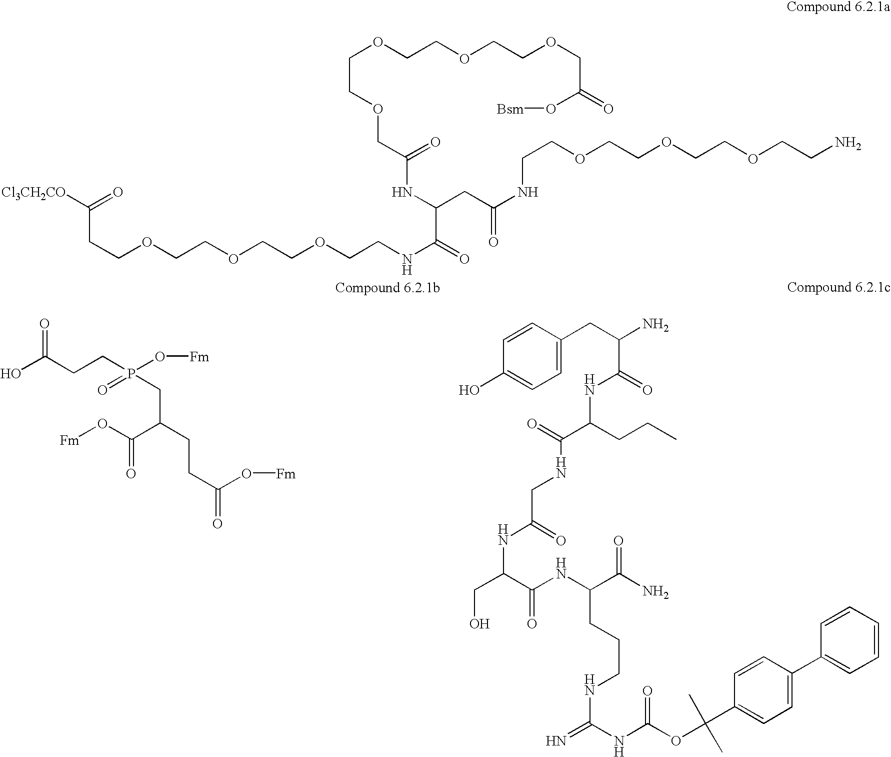

Compound 6.2.1 may be prepared by a multi-step process. Compound 6.2.la and compound 6.2.1 b may be coupled and the product treated with Zn to remove the trichloroethoxycarbonyl group. The product may then be coupled with compound 6.2.1c. Treatment with tris(2-aminoethyl)amine under conditions that will leave the Fm groups intact will then give compound 6.2.1.

Compound 6.2.la may be prepared by a multi-step process. Compound 6.2.0a2 may be coupled to 3-{2-[2-(2-Amino-ethoxy)-ethoxy]-ethoxy}-propionic acid 2,2,2-trichloro-ethyl ester. The product may be treated with acid to cleave the t-butyl ester and then may be coupled to (2-{2-[2-(2-Amino-ethoxy)-ethoxy]-ethoxy}-ethyl)-trityl-amine. The product may then be treated with base to remove the Bsmoc group and coupled to (2-{2-[2-(1, 1 -Dioxo-1H-1 X6-benzo[b]thiophen-2-ylmethoxycarbonylmethoxy)-ethoxy]-ethoxy}-ethoxy)-acetic acid. Acid treatment will remove the trityl group and give compound 6.2.1 a.

Compound 6.2.1b may be prepared by a multistep process. Compound 6.6.1 may be treated with isobutylene and acid (or t-butanol and dicyclohexylcarbodiimide) to give compound 6.2.1b2. The methyl esters may then be hydrolyzed with base. Treatment with (9H-Fluoren-9-yl)-methanol and a condensing agent, such as triisopropylbenzenesulfonyl 3-nitro-1,2,4 triazole and base in an inert solvent will give compound 6.2.1 b3. Treatment with acid will cleave the t-butyl ester and give compound 6.2.1 b.

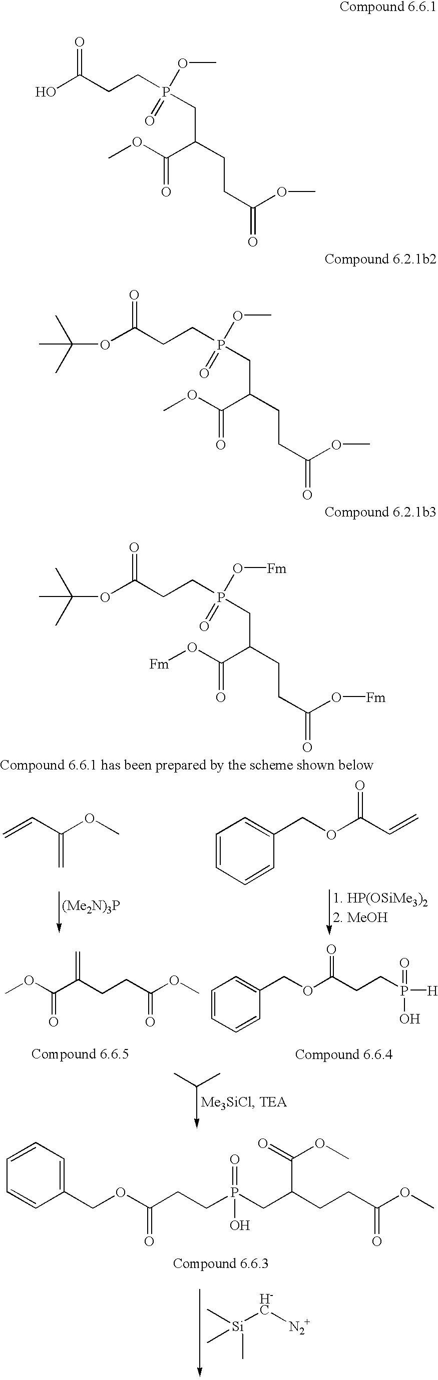

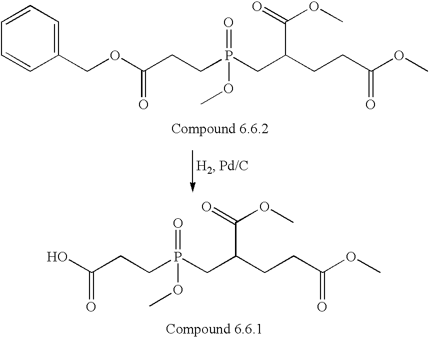

Synthesis of Compound 6.6.5:

Methyl acrylate, 100 ml was heated till reflux and hexamethylphosphorous triamide (HMPT), 3 ml was added in such a rate that the reaction mixture refluxed gently without heating. At the end the mixture was heated at 115° C. for 10 min. Vacuum distillation (0.06 mm Hg) afforded 36.7 g, 38% of compound 6.6.5 as clear liquid, which was characterized by ¹H and 13C NMR.

Synthesis of compound 6.6.4: A mixture of ammonium hypophosphite, 8.3 g, 0.100 mol and hexamethyidisilazane, 18.3 g, 23.4 ml, 0.113 mol was heated and stirred under reflux and nitrogen until the evolution of ammonia ceased. The mixture was cooled, diluted with 50 ml dichloromethane and benzyl acrylate 14.6 g; 0.090 mol was added drop wise on cooling and stirring in such a rate, that the temperature remained −10÷0° C. The reaction mixture was left to reach room temperature, treated with 20 ml methanol, diluted with ethyl acetate, and washed with 1 N HCl. After evaporation of the ethyl acetate, silica gel chromatography (5% acetic acid in dichloromethane) afforded 11.25 g, 55% of compound 6.6.4 as a colorless oil, which was characterized by ¹H and ³¹P NMR.

Synthesis of Compound 6.6.3:

To a solution of compound 6.6.4, 11.25 g, 0.049 mol in 50 ml dichloromethane were added on cooling and stirring under nitrogen at −15° C. triethylamine, 5.9 g, 8.12 ml, 0.059 mol, trimethylchlorosilane, 6.4 g, 7.5 ml, 0.059 mol, and compound 6.6.5,8.44 g, 0.049 mol. Another portion of triethylamine, 5.9 g, 8.12 ml, 0.059 mol, followed by trimethylchlorosilane, 6.4 g, 7.5 ml, 0.059 mol were added in such a rate, that the temperature remained below −10° C. After that the reaction mixture was left overnight at room temperature under nitrogen, diluted with ethyl acetate, washed with 1 N HCl, brine, and extracted with 5% sodium bicarbonate solution, the sodium bicarbonate extract was acidified to ˜pH1 with HCl and extracted with ethyl acetate. Ethyl acetate extract was washed with brine, dried under anhydrous sodium sulfate and evaporated to give 16.7 g, 85% of compound 6.6.3 as light yellow oil, which was characterized by ¹H and ³¹P NMR.

Synthesis of Compound 6.6.2:

A solution of compound 6.6.3, 8.0 g, 0.020 mol in methanol, 100 ml was treated on stirring drop wise with a 2 M solution of (trimethylsilyl)diazomethane in hexanes till a stable yellow color (ca. 45 ml). The reaction mixture was diluted with chloroform, 100 ml and washed with 5% sodium bicarbonate and water. Silica gel chromatography with eluant ethyl acetate afforded 5.3 g of compound 6.6.2 as a clear oil, which was characterized by ¹H and ³¹P NMR.

Synthesis of Compound 6.6.1:

Compound 6.6.2, 6.0 g, 0.014 mol was dissolved in 45 ml methanol and 1 ml acetic acid, and hydrogenated under 600 psi for 72 h in presence of 2.0 g 5% Pd/C in a Parr apparatus. The catalyst was removed by filtration, and the solvent evaporated to give a quantitative yield of compound 6.6.1. The structure and purity of compound 6.6.1 were confirmed by ¹H and ³¹P NMR.

Compound 6.2.1c is based on a known oligopeptide that is readily synthesized by routine techniques of peptide synthesis. The configuration of the amino acids that comprise compound 6.2.1c are L.

### Example 7

Example 7 is a multifunctional drug delivery vehicle that will be selective for prostatic cancer cells that bear both the laminin receptor and PSMA. The drug has a masked biotin moiety as an intracellular transport ligand with a trigger that will be activated by esterase. The toxin indanocine will be liberated following activation of the intracellular trigger by intracellular glutathione or by thioreductases. A wide variety of avidin-intracellular transport ligands may be administered, which will bind to the biotin and transport the drug into the prostate cancer cells by receptor mediated endocytosis.

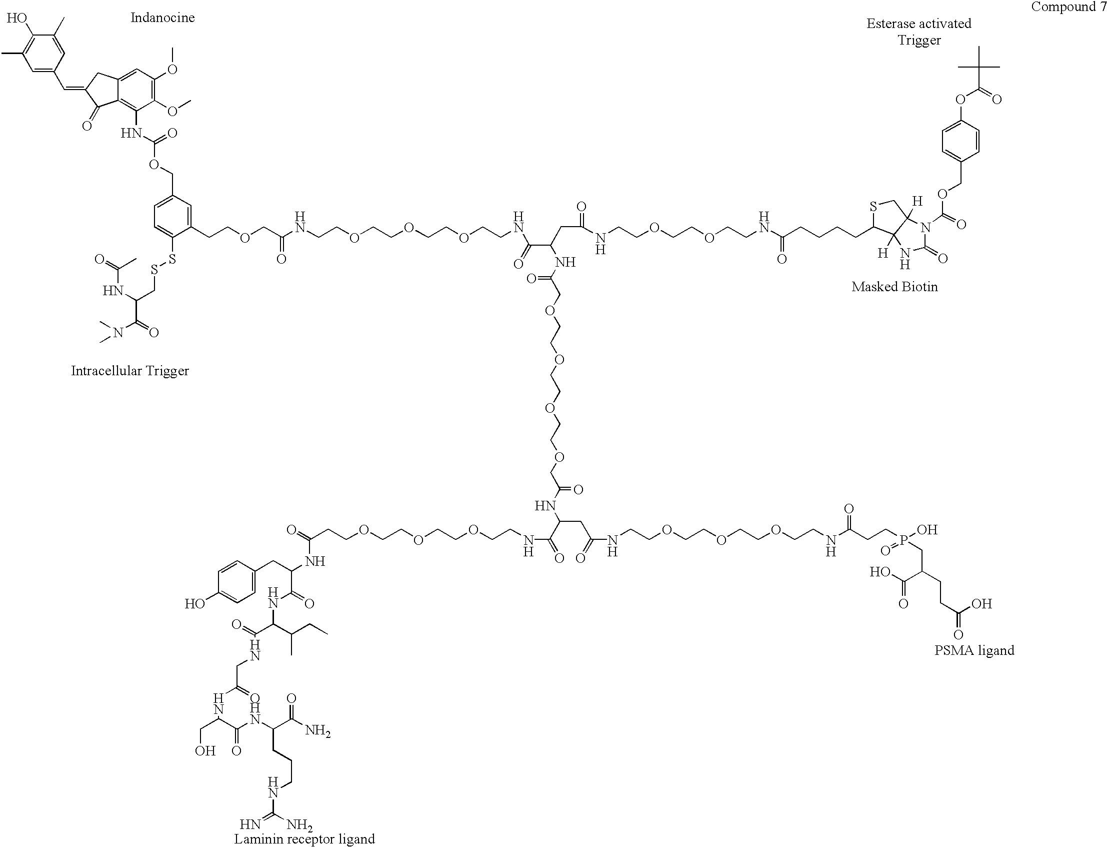

Compound 7 may be synthesized by the methods described for compound 6 by replacing compound 6.2.Ob with compound 7.1.

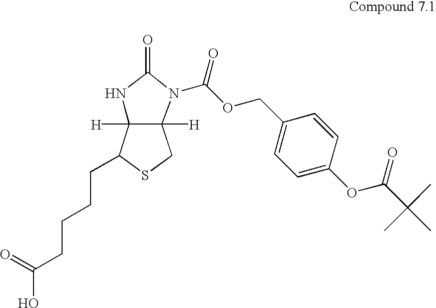

Compound 7.1 may be prepared by treating biotin with a strong base, such as sodium hydride in an inert solvent, such as tetrahydrofuran at low temperature and then reacting with compound 7.1.1 and isolating the product by chromatography. Alternatively, the tetrahydropyranyl ester of biotin may be reacted as described above for biotin and then cleaved by treatment with dilute acid.

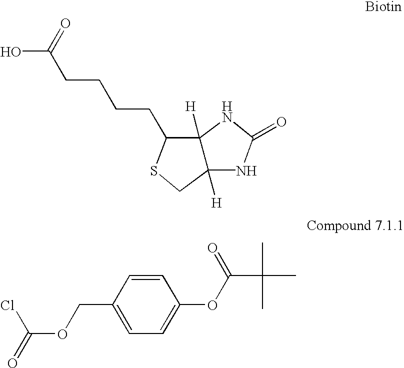

Compound 7.1.1 is readily prepared by the reaction of 2,2-dimethyl-propionic acid 4-hydroxymethyl-phenyl ester with phosgene in toluene. (Treatment of p-hydroxybenzaldehyde with pivaloly chloride and triethylamine in an inert solvent such as methylene chloride gives 2,2-dimethyl-propionic acid 4-formyl-phenyl ester. Catalytic hydrogenation with palladium on carbon yields 2,2-dimethyl-propionic acid 4-hydroxymethyl-phenyl ester.)

### Example 8

Compound 8 is a multifunctional drug delivery vehicle that will be selective for prostatic cancer cells that bear both the laminin receptor and PSMA. The drug has a masked folic acid moiety as an intracellular transport ligand with a clock like time delay trigger that will be activated by esterase. The N-(2-Amino-ethyl)-amide derivative of the toxin BW1843U89 will be liberated following activation of the intracellular trigger by quinone reductase. BW1843U89 inhibits thymidylate synthase at picomolar concentrations. X-ray crystallography of BW1843U89 bound to ecoli thymidylate synthase reveals the carboxylate groups to be free and solvent exposed. Accordingly, the presence of the amino-ethyl group should not impair binding to the thymidylate synthase. The following reference relates to this subject matter: Stout, T. J.; Stroud, R. M., “The Molecular Basis of the Anti-Cancer Therapeutic, BW1843U89, with Thymidylate Synthase at 2.0 Angstroms Resolution,” Protein Data Bank (1996) File 1SYN, the contents of which are incorporated herein by reference in their entirety.

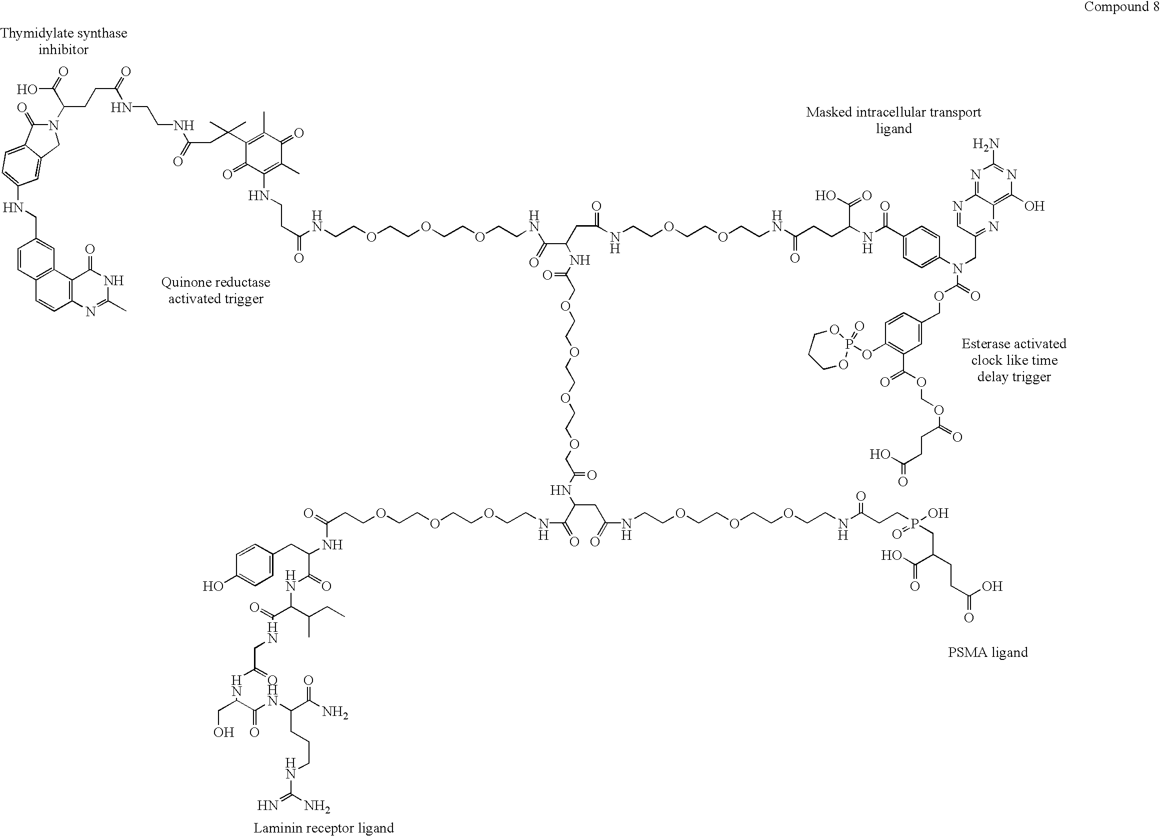

Compound 8 may be synthesized by the methods described for compound 6 by replacing compound 6.2.0.c with compound 8.1.

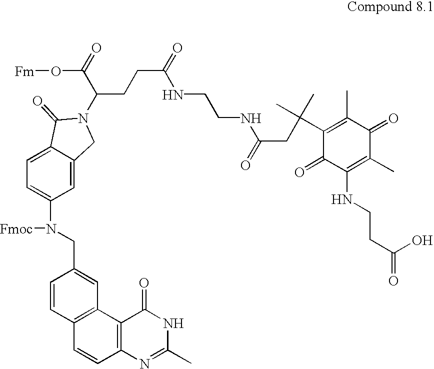

Compound 8.1 may be prepared by coupling compound 8.2 and compound 8.3 and then treating with trifluroacetic acid to cleave the t-butyl ester.

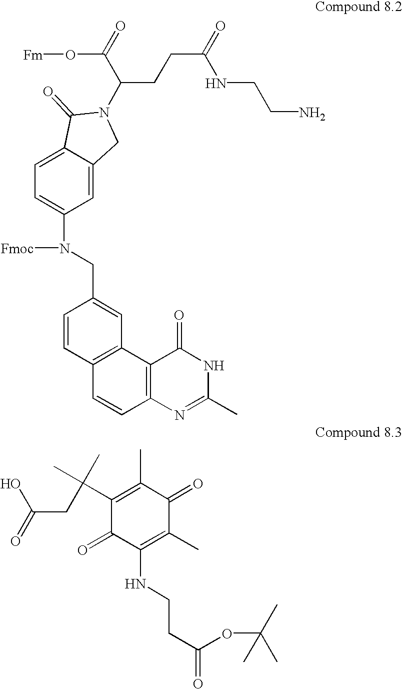

Compound 8.2 as the trifluoroacetate salt, may be prepared by coupling (2-amino-ethyl)-carbamic acid tert-butyl ester with compound 8.2.1 and then removing the t-butyl group with trifluoroacetic acid.

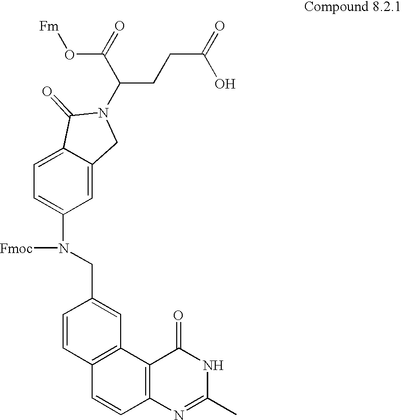

Compound 8.2.1 may be prepared by a multi-step process. The controlled hydrolysis of compound 8.2.1a with aqueous sodium hydroxide in acetonitrile, followed by acidification and chromatography, gives compound 8.2.1b. Treatment with di-t-butyl pyrocarbonate, t-butanol and dimethylaminopyridine in an inert solvent will give compound 8.2.1 c. Treatment with aqueous sodium hydroxide will give compound 8.2.1d. Esterification with 9-H-fluorenyl-9-yl-methanol and a coupling reagent, such as dicylcohexylcarbodiimide, will give compound 8.2.1e. Treatment with trifluoracetic acid and then treatment with one equivalent of (9-H-fluorenyl-9-yl)methyloxycarbonyl chloride in presence of base and in inert solvent will give compound 8.2.1.

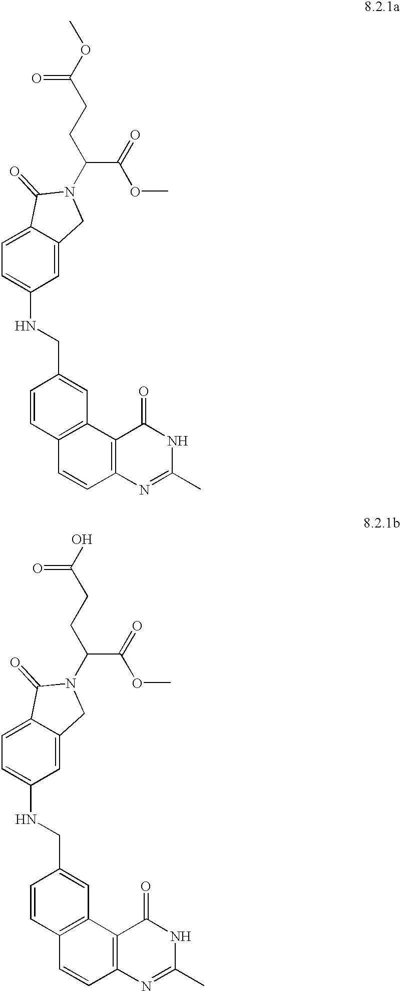

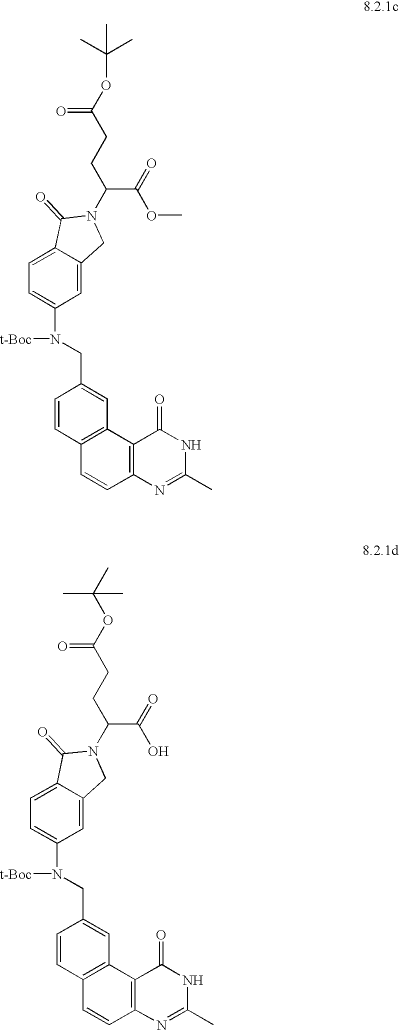

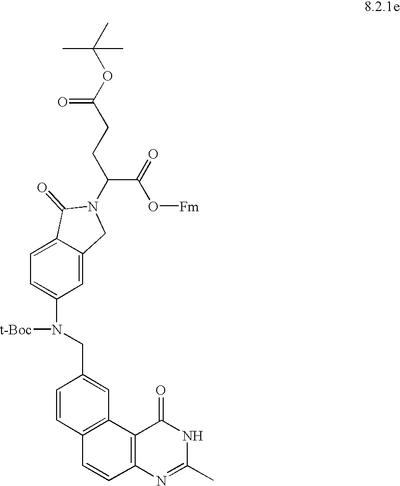

The following references relate to this subject matter: Takeda K., et al., “Dicarbonates: Convenient 4-Dimethylaminopyridine Catalyzed Esterification Reagents,” Synthesis, 1063-1066 (1994), the contents of which are incorporated herein by reference in their entirety.

Compound 8.3 may be prepared by reacting 3-Amino-propionic acid tert-butyl ester with compound 8.3.1.

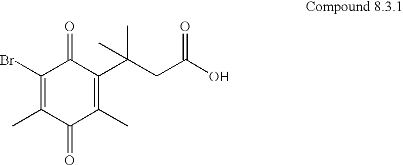

The following reference relates to this subject matter: Carpino L A, et al., “Reductive Lactonization of Strategically Methylated Quinone Propionic Acid Esters and Amides,” J Org Chem, 54:3303-3310 (1989), the contents of which are incorporated herein by reference in their entirety.

### Example 9

Compound 9 is a multifunctional drug delivery vehicle that will be selective for prostatic cancer cells that bear both the laminin receptor and PSMA. The drug has a masked folic acid moiety, as an intracellular transport ligand with a clock like time delay trigger that will be activated by esterase. Hydroxystaurosporine or UCN-01 will be freed upon activation of an intracellular trigger by thiol reductases and hydrolysis of the phosphate group by phosphatases. Hydroxystaurosporine is a potent inhibitor of protein kinases and exhibits synergistic toxicity with a wide range of antineoplastic compounds.

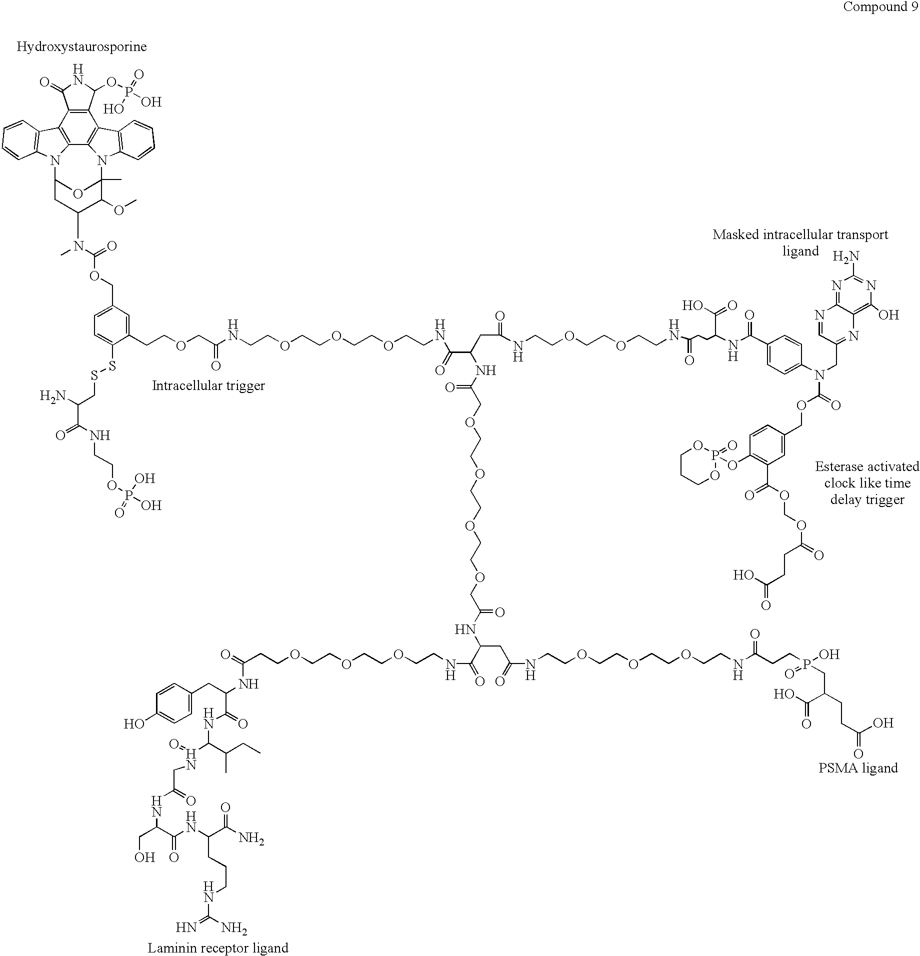

Compound 9 may be prepared by the methods described for compound 6 by replacing compound 6.2.0.c with compound A65.1. (see Example 65)

### Example 10

Compound 10a multifunctional drug delivery vehicle with similar targeting specificity to that of compound 9, the highly potent toxin 2-pyrrolinodoxorubicin, will be liberated upon activation of an intracellular disulfide trigger.

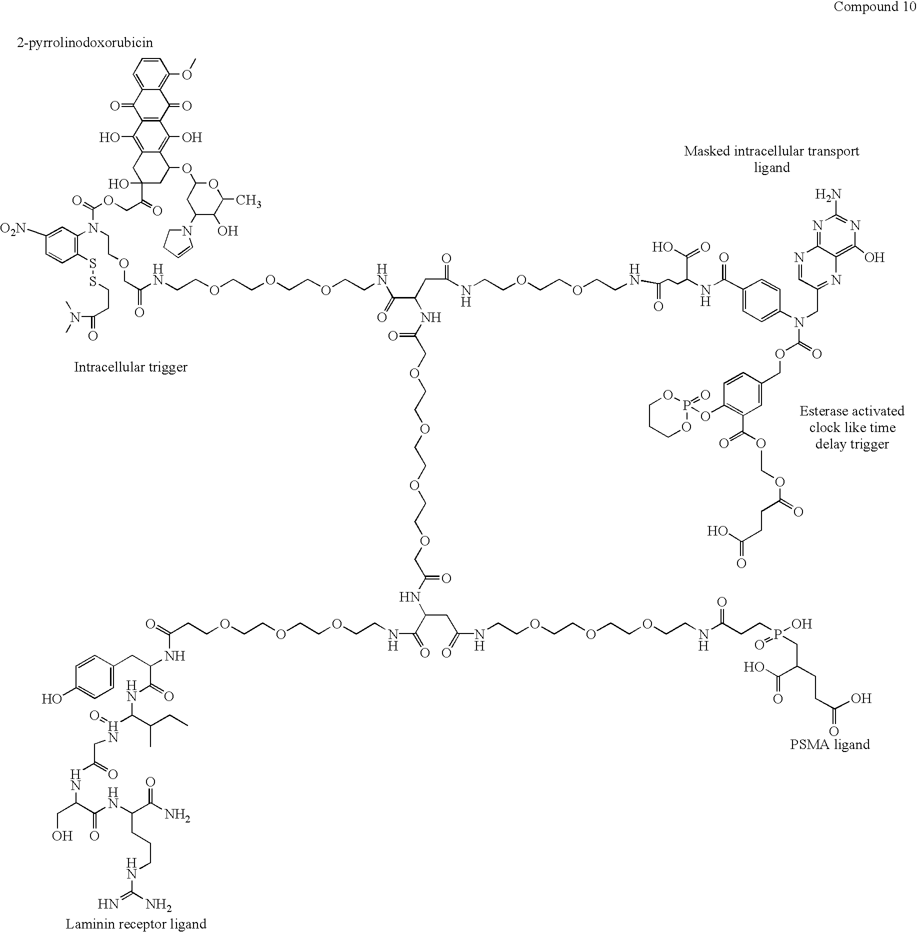

Compound 10 may be prepared by the methods described for compound 6 by replacing compound 6.2.0.c with compound 17.11. (See example 17). Alternatively, compound 10 may be prepared by a route in which 2-pyrrolinodoxorubicin is coupled in the step just prior to the final deblocking step.

### Example 11

Compound 11 is a multifunctional drug delivery vehicle that is similar to compound 10 except it has targeting ligands for urokinase and laminin receptors.

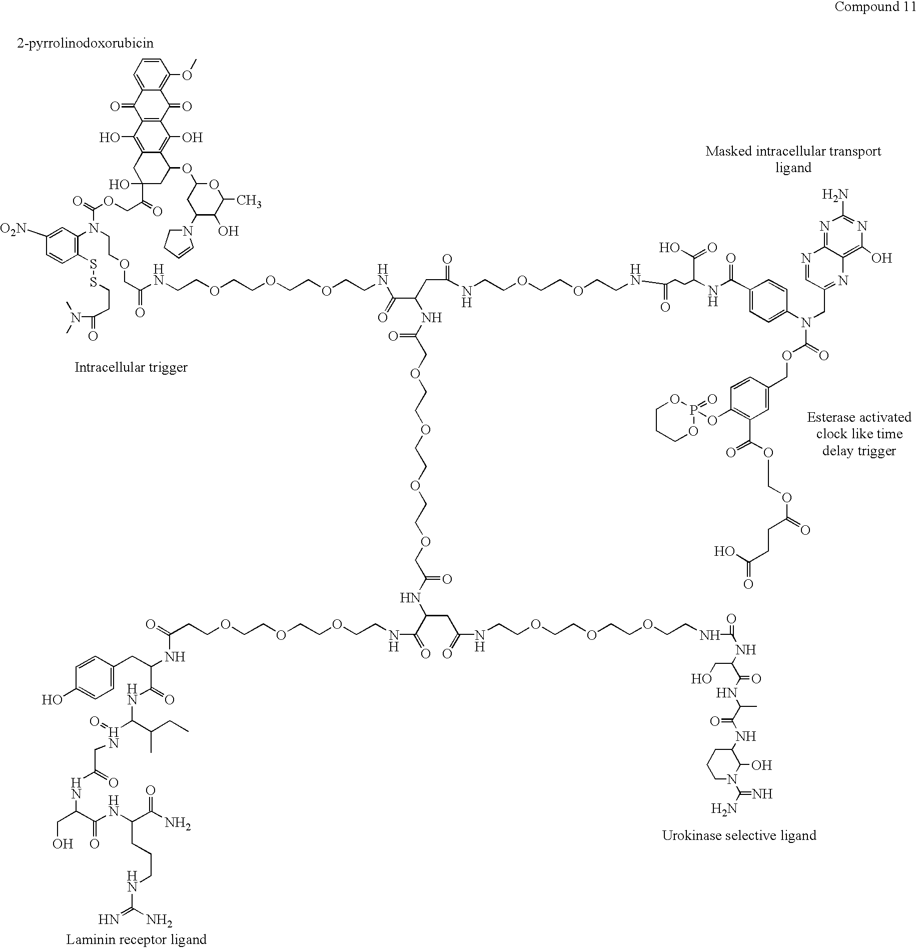

Compound 11 may be prepared by the method described for compound 10 by replacing compound 6.2.1b with compound 11.1 and adding a final deprotection step to remove the silyl protecting groups and treatment with dilute acid to remove the 2-Biphenyl-4-yl-propan-2-oxy-carbonyl protecting group. Methods for the cleavage of t-butyl-dimethylsilyl ethers are well known. The following reference relates to this subject matter: Greene, Theodora W.; Wuts, Peter G. M. (1999) “Protective Groups in Organic Synthesis” John Wiley & Sons, Inc. p 133, the contents of which are incorporated herein by reference in their entirety.

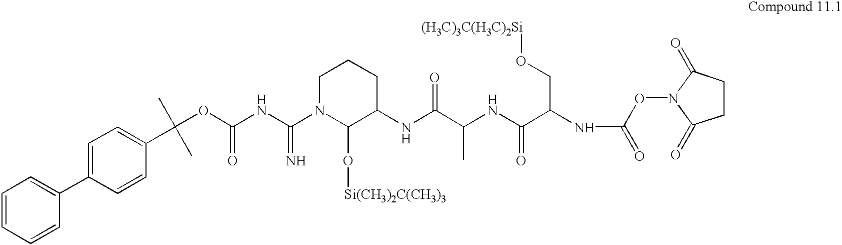

Compound 11.1 may be prepared by treating compound 14.7.8 with t-butyldimethylchlorosilane and base in an inert solvent, followed by treating with base to remove the Fmoc group, followed by treating with N,N′,-disuccinimidyl carbonate in an inert solvent in the presence of pyridine.

### Example 12

Compound 12 is a multifunctional drug delivery vehicle with targeting ligands for PSMA and sigma receptors, both of which are enriched on prostatic cancer cells.

The drug will release Phthalascidin a cytotoxin that has an IC₅₀ in the 0.1-1 nM range. The phathalascidin is linked to the drug complex by a carbamate group that will undergo cleavage upon reduction of a disulfide bond.

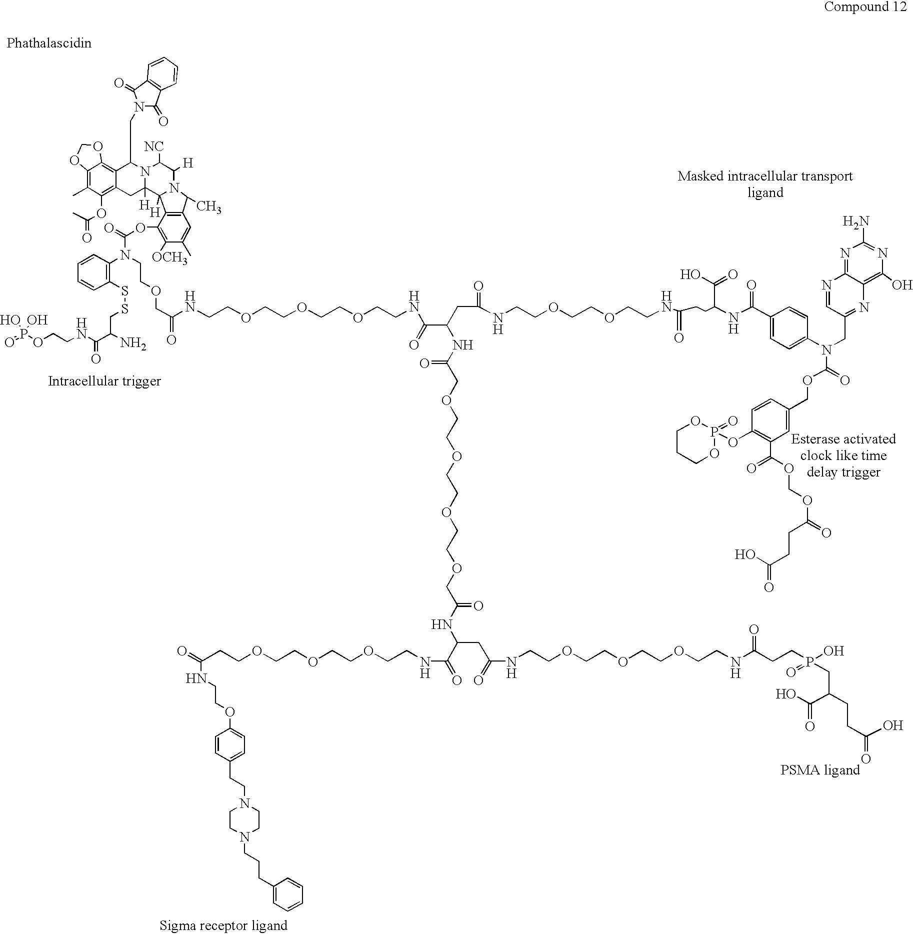

Compound 12 may be prepared by the methods described for compound 6 by replacing compound 6.2.0.c with compound 21.1.2 and compound 6.2.11c with compound 12.1.

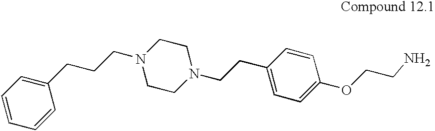

Compound 12.1 may be prepared by a multi-step process. Coupling 1-(3-Phenyl-propyl)-piperazine and [4-(2-Azido-ethoxy)-phenyl]-acetic acid (compound 12.1a) will give 2-[4-(2-Azido-ethoxy)-phenyl]-1-[4-(3-phenyl-propyl)-piperazin-1-yl]-ethanone. This may be reduced with a reagent such as aluminum hydride to give compound 12.1. The following references relate to this subject matter: Zhang Y. et al., “Characterization of Novel N,N′-disubstituted Piperazines as Sigma Receptor Ligands,” J Med Chem, 41(25):4950-7 (1998), the contents of which are incorporated herein by reference in their entirety.

Compound 12.1a may be prepared by a multi-step process. Treating (4-Hydroxy-phenyl)-acetic acid methyl ester with one eqivalent of strong base and 1 equivalent of ethylene oxide in an inert solvent will give [4-(2-Hydroxy-ethoxy)-phenyl]-acetic acid methyl ester. Treating with tosyl chloride and base in an inert solvent will give {4-[2-(Toluene-4-sulfonyloxy)-ethoxy]-phenyl}-acetic acid methyl ester. Treating with lithium azide in an inert solvent will give [4-(2-Azido-ethoxy)-phenyl]-acetic acid methyl ester. Hydrolysis of the methyl ester will give compound 12.1a.

### Example 13

Compounds 13a and 13b are an example of a versatile set of linkers that may be employed in the synthesis of a large number of multifunctional drug delivery vehicles. Compound 13a and 13b may be substituted with a large variety of ligands, triggers, and effector groups and then joined together. The linker contains a phosphonate group to increase water solubility of the ultimate multifunctional delivery vehicles.

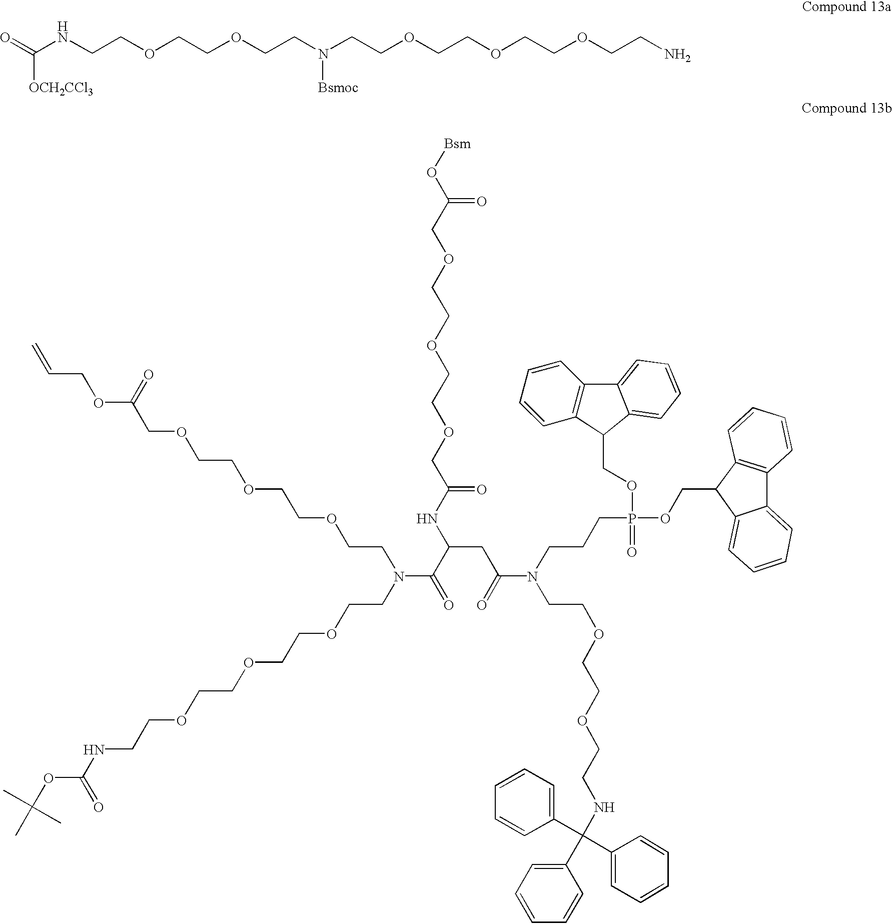

Compound 13a may be prepared by a multi-step process. Treating {2-[2-(2-Amino-ethoxy)-ethoxy]-ethyl}-[2-(2-{2-[2-(trityl-amino)-ethoxy]-ethoxy}-ethoxy)-ethyl]-amine (Compound 13a. 1) with one equivalent of 2,2,2 trichloroethyl N-succinimidyl carbonate in an inert solvent will give [2-(2-{2-[2-(2-{2-[2-(Trityl-amino)-ethoxy]-ethoxy}-ethoxy)-ethylamino]-ethoxy}-ethoxy)-ethyl]-carbarmic acid 2,2,2-trichloro-ethyl ester. Treating with (1,1-dioxo-1H-1λ6-benzo[b]thiophen-2-yl)-methyl chloroformate and a base such as pyridine followed by acid treatment to remove the trityl group will give compound 13a as the salt.

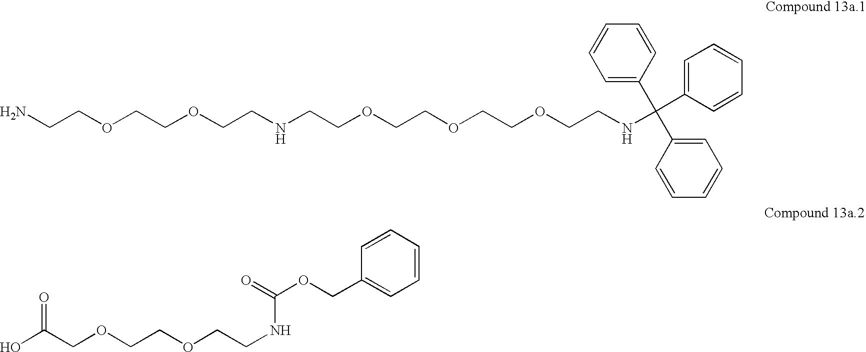

Compound 13a.1 may be prepared by a multi-step process. Treating 2-{2-[2-(2-Amino-ethoxy)-ethoxy]-ethoxy}-ethylamine with one equivalent of trityl chloride and base in an inert solvent will give, after purification, 2-(2-{2-[2-(Trityl-amino)-ethoxy]-ethoxy}-ethoxy)-ethylamine. This may then be coupled to [2-(2-Benzyloxycarbonyl-amino-ethoxy)-ethoxy]-acetic acid (Compound 13.a.2) and reduced with an agent such as lithium aluminum hydride in an inert solvent to give compound 13.a1.

Compound 13a.2 may be prepared by a multi-step process. Oxidation of 2-[2-(2-chloro-ethoxy)-ethoxy]-ethanol with Pt on carbon or platinum dioxide in water with air will give [2-(2-Chloro-ethoxy)-ethoxy]-acetic acid. Treatment with lithium azide, followed by catalytic hydrogenation with Pd on carbon, followed by reaction with benzyl chloroformate under Schotten-Bauman conditions, will give compound 13a.2.

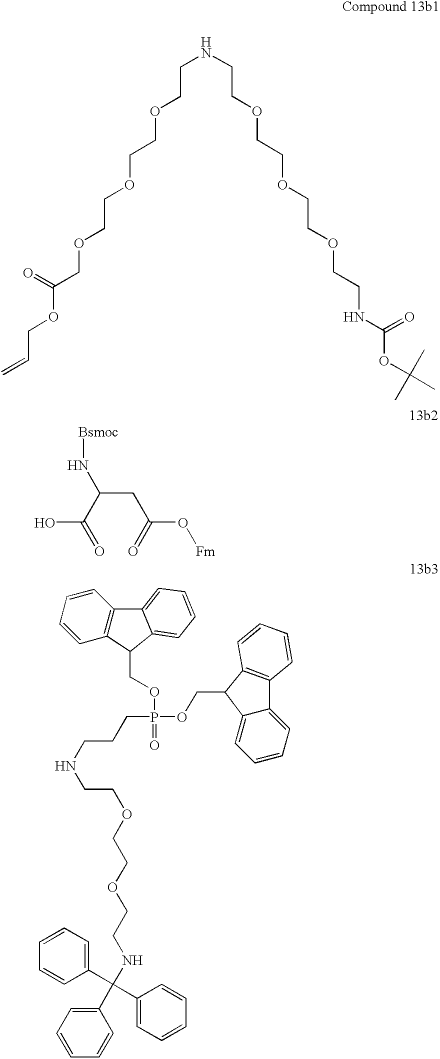

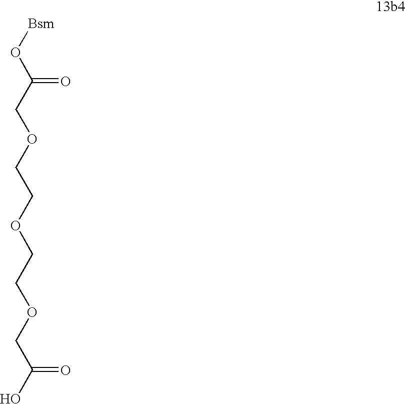

Compound 13b1 and compound 13b2 may be coupled and the product treated with a hindered base to selectively cleave the Fm ester. The product may then be coupled with compound 13b3 and treated with tris(2-aminoethyl) amine to cleave the Bsmoc group under conditions that will leave the Fmoc and Fm esters intact. The product may then be coupled to compound 13b4 to give compound 13b. The following references relate to this subject matter: Carpino L. A., et al., “New Family of Base- and Nucleophile-Sensitive Amino-Protecting Groups. A Michael-Acceptor-Based Deblocking Process. Practical Utilization of the 1,1-Dioxobenzo[b]thiophene-2-ylmethyloxycarbonyl (Bsmoc) Group,” J Am Chem Soc, 119:9915-9916 (1997); Carpino L. A., et al., “The 1,1-Dioxobenzo[b]thiophene-2-ylmethyloxycarbonyl (Bsmoc) Amino-Protecting Group,” J Org Chem, 64:4324-4338 (1999), the contents of which are incorporated herein by reference in their entirety.

Compound 13b1 may be prepared by a multi-step process. Reacting {2-[2-(2-chloro-ethoxy)-ethoxy]-ethoxy}-acetic acid and (2-{2-[2-(2-Amino-ethoxy)-ethoxy]-ethoxy}-ethyl)-trityl-amine with a base in an inert solvent will give, after purification, [2-(2-{2-[2-(2-{2-[2-(Trityl-amino)-ethoxy]-ethoxy}-ethoxy)-ethylamino]-ethoxy}-ethoxy)-ethoxy]-acetic acid. Treatment with (9H-Fluoren-9-yl)-methyl chloroformate and base in an inert solvent, followed by treatment with dicyclohexylcarbodiimide and allyl alcohol will give {2-[2-(2-{(9H-Fluoren-9-ylmethoxycarbonyl)-[2-(2-{2-[2-(trityl-amino)-ethoxy]-ethoxy}-ethoxy)-ethyl]-amino}-ethoxy)-ethoxy]-ethoxy}-acetic acid allyl ester. Treatment with acid followed by treatment with di-t-butyl pyrocarbonate and in an inert solvent will give [2-(2-{2-[(2-{2-[2-(2-tert-Butoxycarbonylamino-ethoxy)-ethoxy]-ethoxy}-ethyl)-(9H-fluoren-9-yl methoxy-carbonyl)-amino]-ethoxy}-ethoxy)-ethoxy]-acetic acid allyl ester. Treatment with base to remove the Fmoc group will give compound 13b1.

Compound 13b2 may be prepared by treating L-aspartic acid α-t-butyl ester with (1,1-Dioxo-1H-1λ6-benzo[b]thiophen-2-yl)-methyl chloroformate and base in an inert solvent or under Schotten-Bauman conditions, and then treating the product with dicyclohexylcarbodiimide and (9H-Fluoren-9-yl)-methanol, and then treating with acid to cleave the t-butyl ester.

Compound 13b3 may be prepared by a multi-step process. Treating [2-(2-Chloro-ethoxy)-ethoxy]-acetic acid with lithium azide in an inert solvent will give [2-(2-Azido-ethoxy)-ethoxy]-acetic acid. Coupling with 3-amino-propan-1-ol will give 2-[2-(2-Azido-ethoxy)-ethoxy]-N-(3-hydroxy-propyl)-acetamide. Reducing with a reagent, such as lithium aluminum hydride in an inert solvent will give 3-{2-[2-(2-Amino-ethoxy)-ethoxy]-ethylamino}-propan-1-ol. Treating with di-t-butyl pyrocarbonate and in an inert solvent will give {2-[2-(2-tert-Butoxycarbonylaminoethoxy)-ethoxy]-ethyl}-(3-hydroxy-propyl)-carbamic acid tert-butyl ester Treatment with triphenylphosphine and carbon tetrachloride will give {2-[2-(2-tert-Butoxycarbonylamino-ethoxy)-ethoxy]-ethyl}-(3-chloro-propyl)-carbamic acid tert-butyl ester. An Arbuzov reaction with tris (trimethylsilyl) phosphite will give compound 13.b3.1.

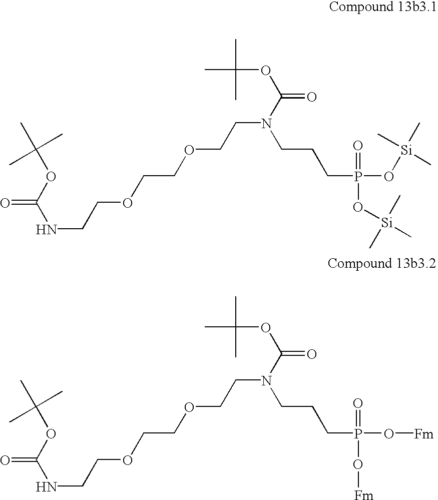

Treating compound 13.b3.1 with oxalyl chloride and a catalytic amount of dimethylformamide in an inert solvent and removing the chlorotrimethylsilane exvacuo will yield the phosphonic dichloride. Reacting with (9H-Fluoren-9-yl)-methanol in the presence of a base such as triethylamine will give compound 13b3.2. Alternatively, the silyl esters may be hydrolyzed and the resulting phosphonate may be esterified with (9H-Fluoren-9-yl)-methanol using an agent, such as triisopropylbenzenesulfonyl 3-nitro-1,2,4 triazole and base in an inert solvent to give compound 13b3.2. Treatment with acid will remove the t-Boc groups. Treatment with one equivalent of trityl chloride and base in an inert solvent will give compound 13b3.

Compound 13b4 may be prepared by treating [2-(2-carboxymethoxy-ethoxy)-ethoxy]-acetic acid with one equivalent of (9H-Fluoren-9-yl)-methanol and a reagent such as dicyclohexylcarbodiimide in an inert solvent, followed by chromatographic separation.

### Example 14

Example 14 is a multifunctional drug delivery vehicle with targeting ligands selective for urokinase, sigma receptors, and matrix metalloproteinases (1,2,3,9, and MT-MMP-1). The drug has a masked folic acid group as intracellular transport ligand with a clock like time deay trigger, which is unmasked by nonspecific esterase. A highly cytotoxic ellipticine analog will be released after activation of an intracellular trigger by thioreductase. The following references relate to this subject matter: Bisagni E., et al., “Synthesis of 1-Substituted Ellipticines by a New Route to Pyrido[4,3-b]-carbazoles,” JCS Perkin I, 1706-1711 (1978); Czerwinski G., et al., “Cytotoxic Agents Directed to Peptide Hormone Receptors: Defining the Requirements for a Successful Drug,” Proc Natl Acad Sci USA, 95:11520-11525 (1998), the contents of which are incorporated herein by reference in their entirety.

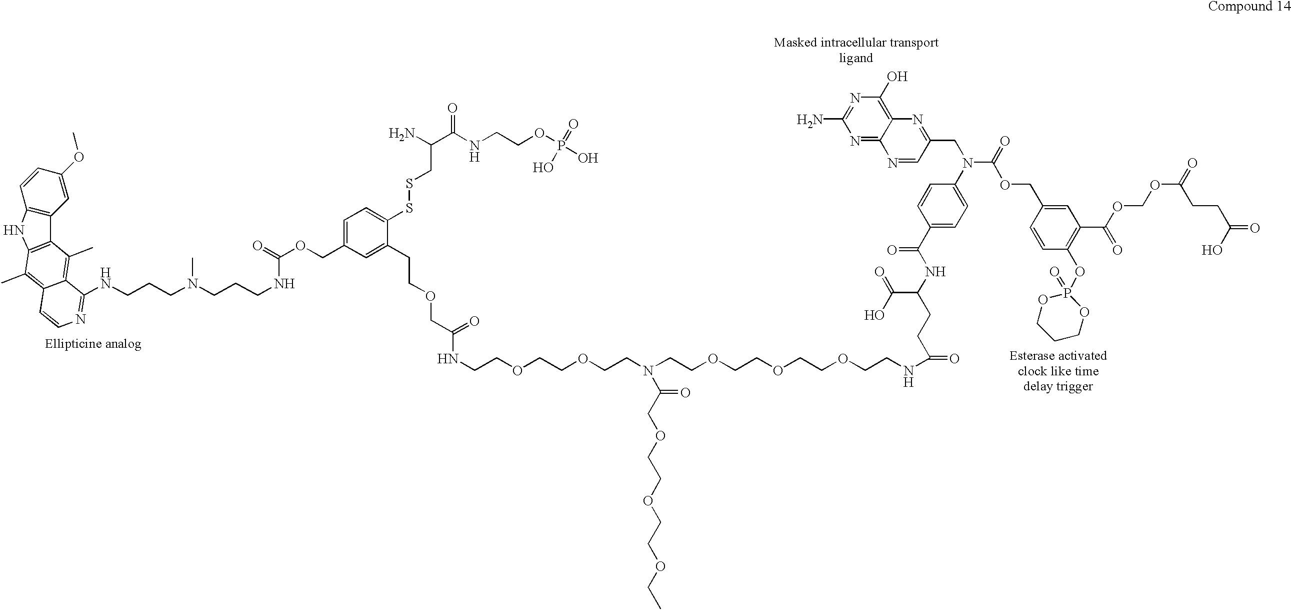

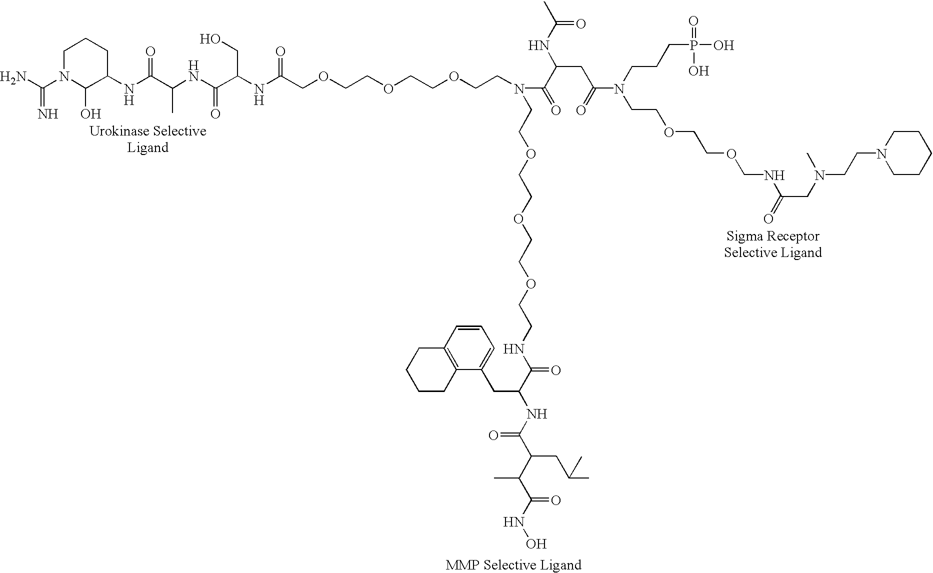

Synthesis of Compound 14

Coupound 14 may be prepared by coupling compound 14.1a and compound 14.1b and then treating with base to remove the Fm and Fmoc groups, and then treating with a reagent such as tetrabutyl ammonium fluoride or pyridine-HF to remove the silyl protecting groups. The 2-Biphenyl-4-yl-propan-2-oxy-carbonyl protecting group may be removed by treatment with dilute acid.

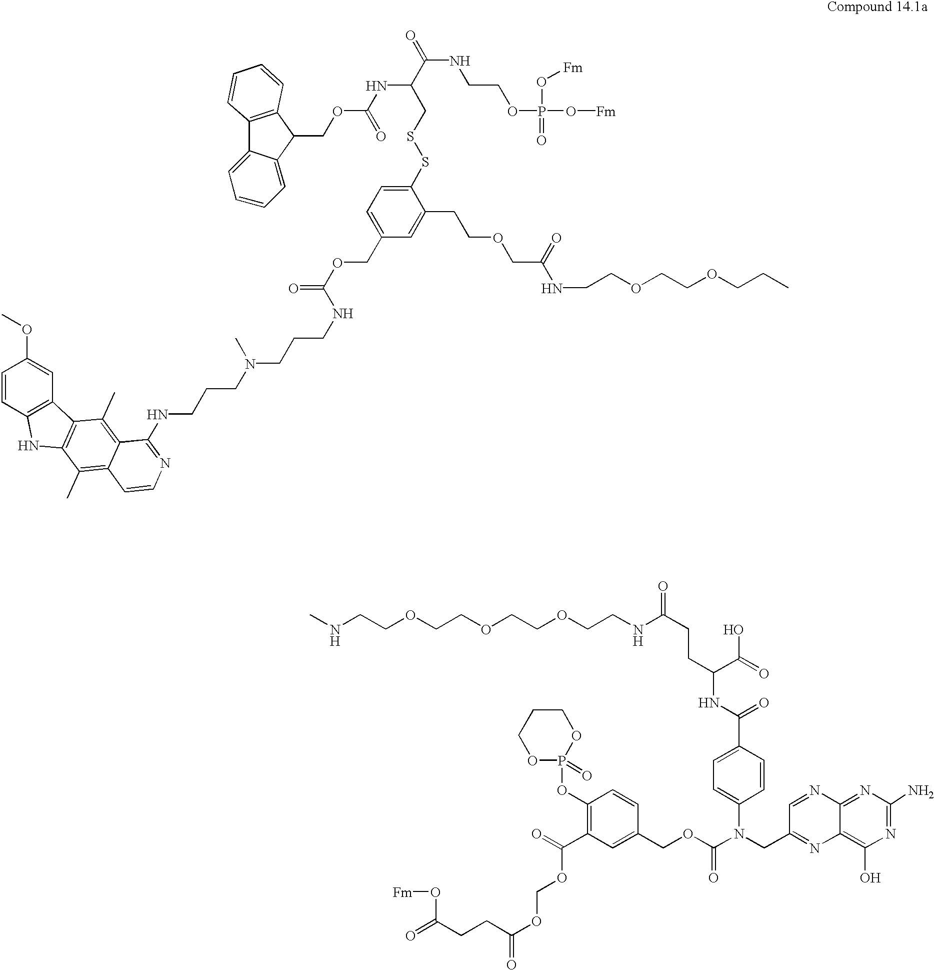

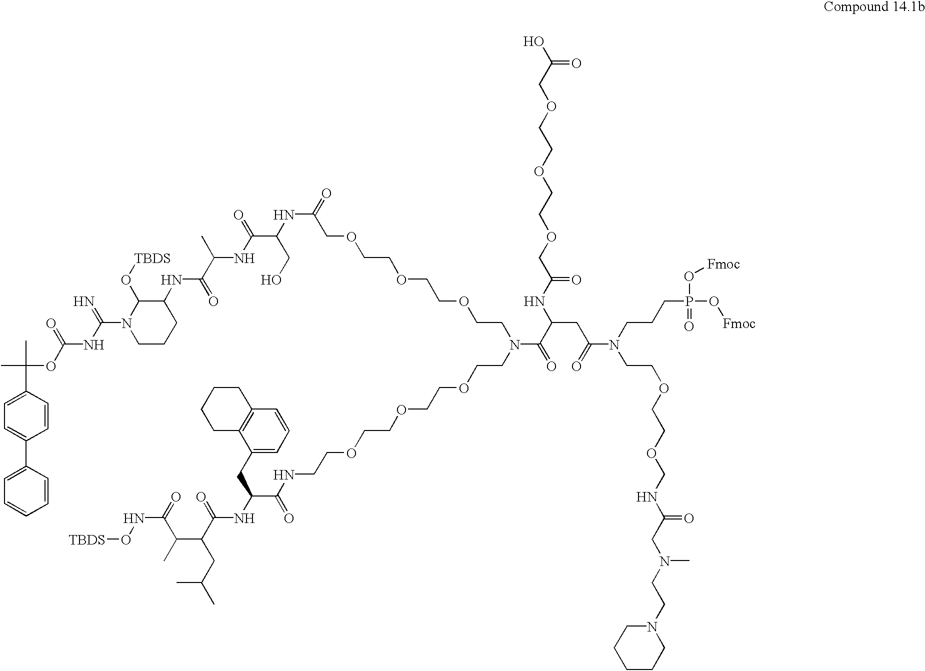

Compound 14.1a may be prepared by a multi-step process. Compound 13a may be coupled to compound 6.2.0b and then treated with Zn and acid to remove the trichloroethoxycarbonyl group. The product may then be coupled to compound 14.11. The product may then be treated with tris(2-aminoethyl)amine under conditions that will leave the Fmoc and Fm esters intact to give compound 14.1a.

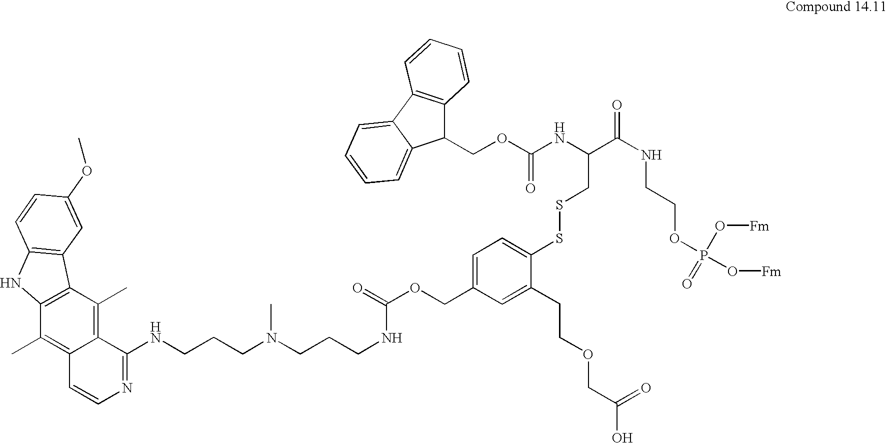

Compound 14.11 may be prepared by reacting compound 14.11.1 and compound 14.11.2 in an inert solvent and then selectively cleaving the Bsm ester with tris(2-aminoethyl)amine under conditions that will leave the Fm esters intact.

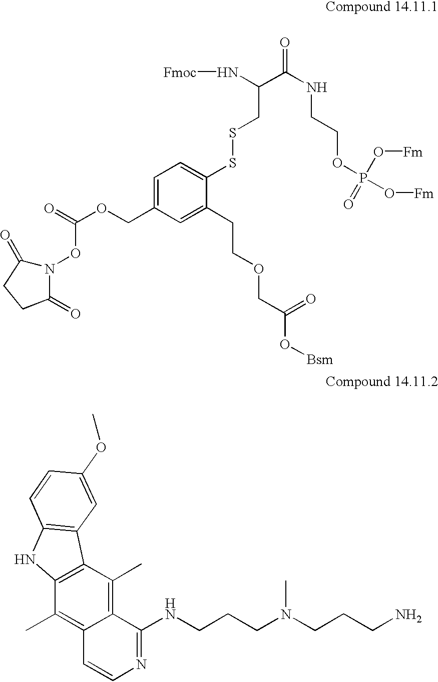

Compound 14.11.1 may be prepared by reacting the corresponding benzylic alcohol (compound 14.11.3) with N,N′, disuccinimidyl carbonate in an inert solvent in the presence of pyridine. The following reference relates to this subject matter: Manoharan M., et al., “N-(2 Cyanoethoxycarbonyloxy)succinimide: A New Reagent for Protection of Amino Groups in Oligonucleotides,” J Org Chem, 63:6468-6472 (1999), the contents of which is incorporated herein by reference in its entirety.

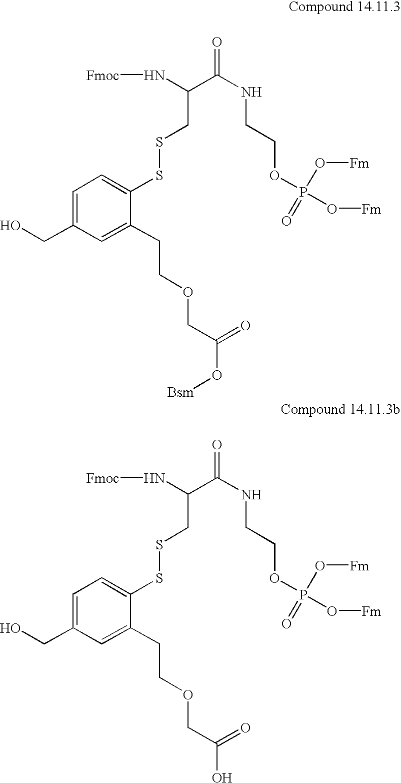

Compound 14.11.3 may be prepared by treating compound 14.11.3b with trityl chloride and a base such as pyridine, and the reacting with dicyclohexylcarbodiimide and (1,1-Dioxo-1H-1λ6-benzo[b]thiophen-2-yl)-methanol in an inert solvent, and then removing then removing the trityl group with acid.

Compound 14.11.3b may be prepared by forming the mixed disulfide between compound 14.11.4 and compound 6.2.0b2.

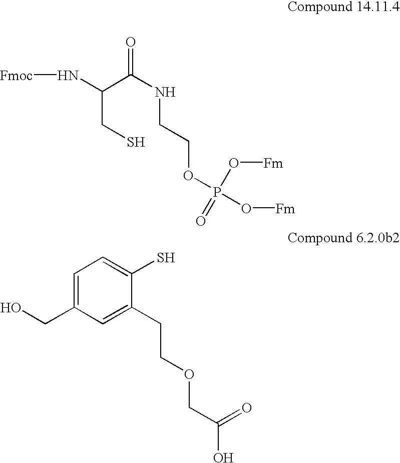

The mixed disulfide may be formed by a variety of methods, such as treatment of compound 14.11.4 with one equivalent of sulfuryl chloride and pyridine in an inert solvent at −78° C. to form the the sulfenyl chloride which can then be reacted without isolation at −78° C. with compound 6.2.0b2. The following references relate to this subject matter: Derbesy G.; Harpp D. N., “A Simple Method to Prepare Unsymmetrical Di- Tri- and Tetrasulfides,” Tetrahedron Letters, 35(30):5381-5384 (1994), the contents of which are incorporated herein by reference in their entirety.

Compound 14.11.4 may be prepared by coupling Fmoc-S-p-methoxytrityl-L-cysteine and 2-amino ethanol, reacting the product with phosphorochloridic acid bis-(9H-fluoren-9-ylmethyl) ester and a base such as triethylamine in an inert solvent, and finally treating with acid to cleave the methoxytrityl protecting group.

Phosphoric acid tris-(9H-fluoren-9-ylmethyl) ester may be prepared by reacting phosphorus oxychloride and (9H-Fluoren-9-yl)-methanol in an inert solvent in the presence of a base such as triethylamine. Treating with one equivalent of a strong base will give phosphoric acid bis-(9H-fluoren-9-ylmethyl) ester. Treatment with chlorotrimethylsilane and triethylamine in an inert solvent followed by treatment with oxalyl chloride and a catalytic amount of dimethylformamide will give phosphorochloridic acid bis-(9H-fluoren-9-ylmethyl) ester.

Compound 14.1b may be prepared by a multi-step process. Compound 13b may be treated with acid to selectively remove the trityl group. The product may then be coupled to compound 14.5. The product may then be treated with trifluoroacetic acid to remove the t-Boc group. The product may then be coupled to compound 14.6; the product may then be treated with Pd(0) to cleave the allyl ester. The product may then be coupled to compound 14.7. Selectively cleaving the Bsm ester with tris(2-aminoethyl)amine under conditions that will leave the Fm esters intact will give compound 14b. The following references relate to this subject matter: Genêt J. P., et al., “Practical Palladium-Mediated Deprotective Method of Allyloxycarbonyl in Aqueous Media,” Tetrahedron, 50(2):497-503 (1994); Kunz H.; Unverzagt C., “The Allyloxycarbonyl (Aloc) Moiety-Conversion of an Unsuitable into a Valuable Amino Protecting Group for Peptide Synthesis,” Angew Chem Int Ed Engl, 23 (6):436-437 (1984); Genêt J. P., et al., “A General and Simple Removal of the Allyloxycarbonyl Protecting Group by Palladium-Catalyzed Reactions Using Nitrogen and Sulfur Nucleophiles,” Synlett, 680-682 (1993), the contents of which are incorporated herein by reference in their entirety.

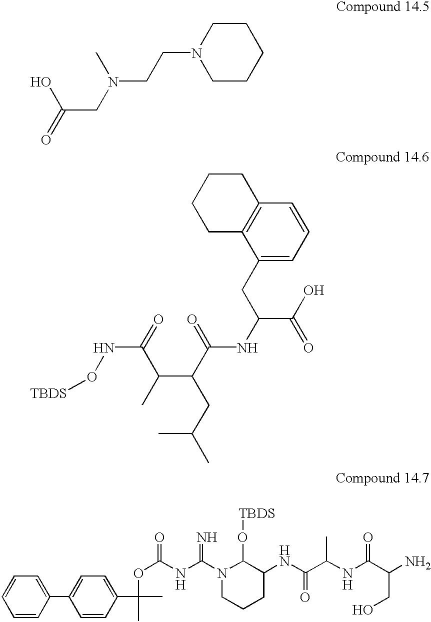

Compound 14.5 may be prepared by the reaction of ethyl bromoacetate and methyl-(2-piperidin-1-yl-ethyl)-amine followed by hydrolysis of the ethyl ester.

Compound 14.6 may be prepared by a multi-step procedure. Compound 14.6.1 and compound 14.6.2 may be coupled to give 14.6.3.

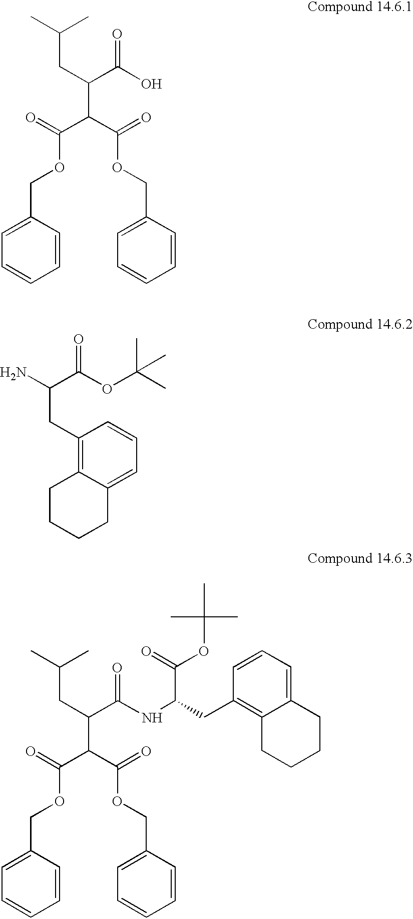

Catalytic hydrogenation of compound 14.6.3 followed by treatment with formaldehyde and piperidine will give compound 14.6.4. Catalytic hydrogenation of compound 14.6.4 with Pd will give compound 14.6.5. Esterfication with 4-nitrophenol followed by treatment with trifluoracetic acid to cleave the t-butyl ester will give compound 14.6.6. Treatment with )-tert-butyldimethylsilyl hydroxylamine will give compound 14.6. The following references relate to this subject matter: Yamamoto M., et al., “Inhibition of Membrane-Type 1 Matrix Metalloproteinase by Hydroxamate Inhibitors: An Examination of the Subsite Pocket,” J Med Chem, 41:1209-1217 (1998), the contents of which are incorporated herein by reference in their entirety.

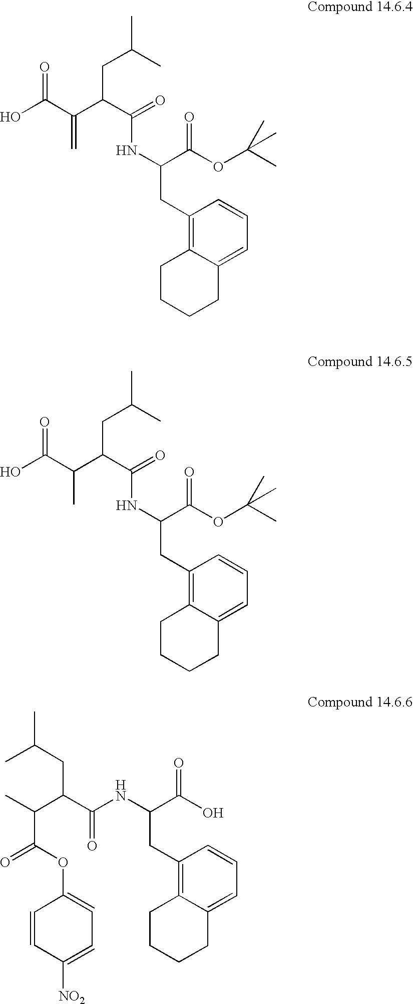

Compound 14.7 may be prepared by a multi-step procedure.

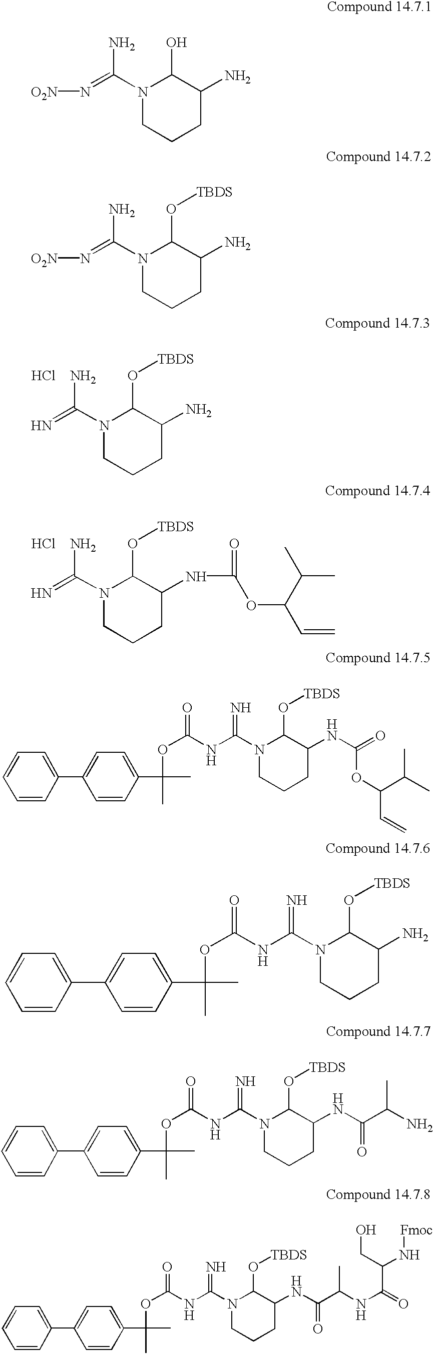

Compound 14.7 may be prepared by a multi-step procedure. Compound 14.7.1 may be treated with t-butyidimethylchlorosilane and base in an inert solvent to give compound 14.7.2. Catalytic hydrogenation with Pd on carbon in the presence of HCl will give compound 14.7.3. Treatment with 4,6-dimethyl-2-(1-isopropylallyl-oxycarbonylthio)pyrimidine and base in an inert solvent will give compound 14.7.4. Treatment with 1 equivalent of strong base in an inert solvent and a reagent such as 4-(1-Biphenyl-4-yl-1-methyl-ethoxycarbonyloxy)-benzoic acid methyl ester will give compound 14.7.5. Removal of the isopropylallyloxycarbonyl protecting group with Pd(0) will give compound 14.7.6. Compound 14.7.6 may then be coupled with Fmoc protected L-alanine and treated with base to remove the Fmoc group and give compound 14.7. Coupling with Fmoc protected D-serine will give compound 14.7.8. Treatment with base to remove the Fmoc group will give compound 14.7. The following references relate to this subject matter: Tamura S. Y., et al., “Synthesis and Biological Activity of Peptidyl Aldehyde Urokinase Inhibitors,” Bioorg Med Chem Lett, 10:983-987 (2000); Minami I., et al., “1-Isopropylallyloxycarbonyl (IPAoc) as a Protective Group of Amines and its Deprotection Catalysed by Palladium-Phosphine Complex,” Tetrahedron Let, 28(24):2737-2740 (1987), the contents of which are incorporated herein by reference in their entirety.

### Example 15

Compound 15 has targeting specificity similar to compound 14 by releases the highly potent cytotoxin mitoxantrone.

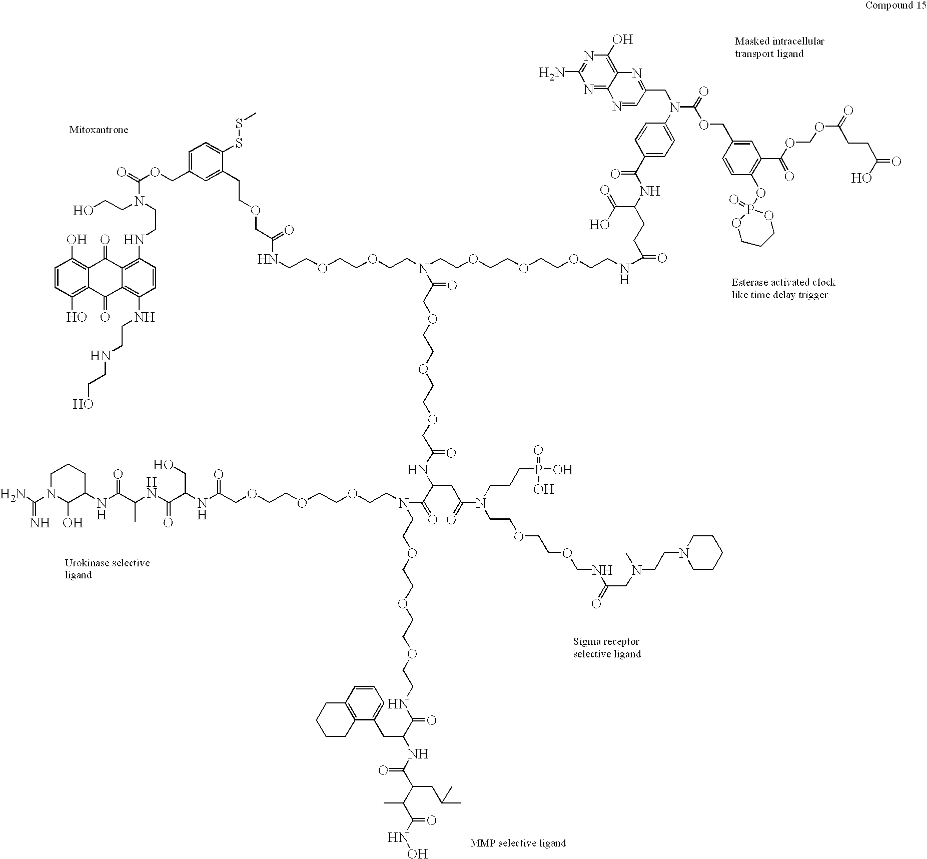

Compound 15 may be prepared by the methods described for compound 14 by replacing compound 14.11 with compound 15.1.

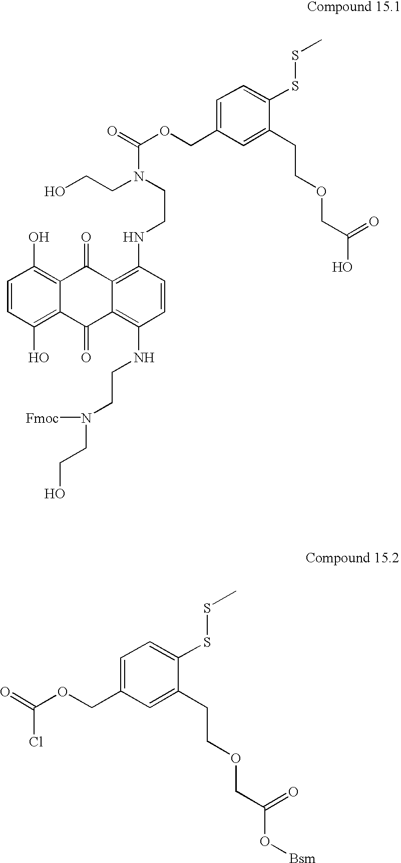

Compound 15.1 may be prepared by reacting mono Fmoc mitoxantrone or ((2-{5,8-dihydroxy-4-[2-(2-hydroxy-ethylamino)-ethylamino]-9,10-dioxo-9,10-dihydroanthracen-1-ylamino}-ethyl)-(2-hydroxy-ethyl)-carbamic acid 9H-fluoren-9-ylmethyl ester) with compound 15.2 in an inert solvent in the presence of a base, such as pyridine, and then treating with tris(2-aminoethyl)amine to cleave the Bsm ester under conditions that will leave the Fmoc group intact.

Compound 15.2 may be prepared by the methods described for the synthesis of compound 6.2.0b1 by replacing N-acetyl-L-cysteine N-N-dimethylamide with methanethiol.

### Example 16

Example 16 is similar to compound 14, but has a detoxification trigger that will be activated by aryl sulfatase. Cleavage of the sulfate ester will trigger the separation of the intracellular trigger-toxin moiety from the intracellular transport group and functionally detoxify the drug. The functional detoxification will result from impaired cellular penetration of the liberated ionic ellipiticine-intracellular trigger complex. This drug may be used with an aryl sulfatase-targeting complex that is specific for vital normal cells.

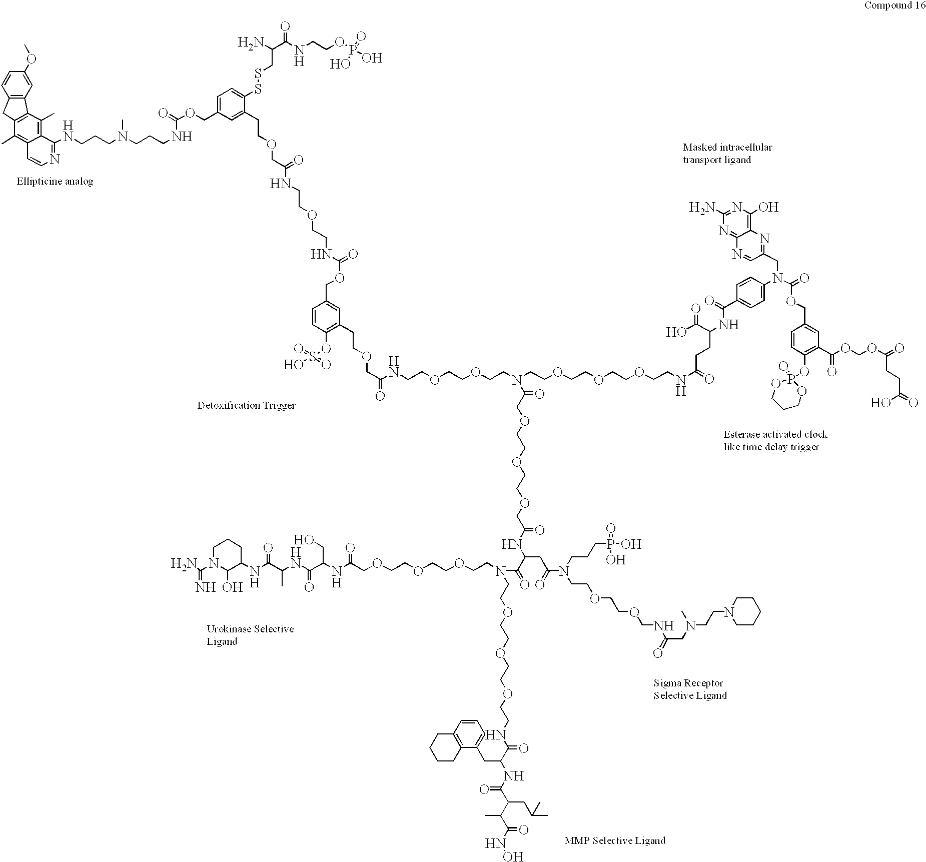

Compound 16 may be prepared by the methods described for compound 14 by replacing compound 14.11 with compound 16.1.

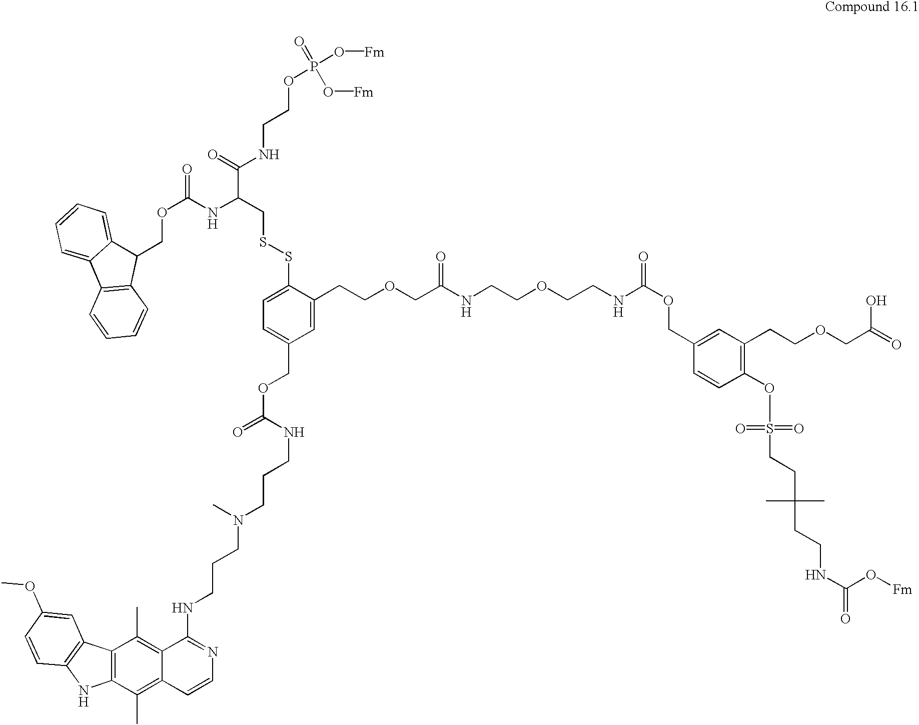

Compound 16.1 may be prepared by coupling compound 16.2 and compound 14.11 and then selectively cleaving the Bsm ester with tris(2-aminoethyl)amine under conditions that will leave the Fm esters intact.

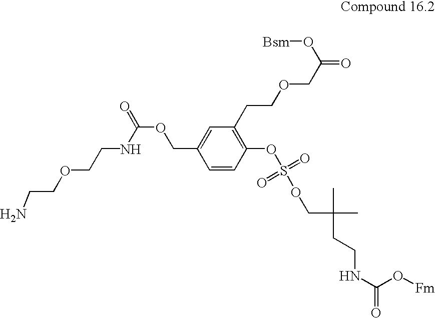

Compound 16.2 may be prepared by reacting 2-[2-(Trityl-amino)-ethoxy]-ethylamine and compound 16.3 in an inert solvent in the presence of a base such as pyridine and then treating with acid to remove the trityl protecting group.

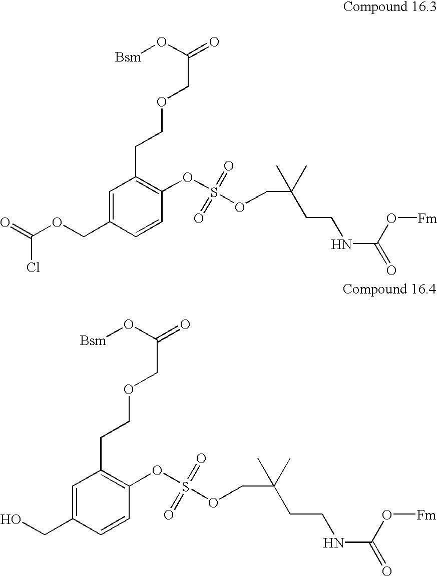

Compound 16.3 may be prepared by treating compound 16.4 with phosgene in an inert solvent.

Compound 16.4 may be prepared by a multi-step process. Compound 16.5 and compound 16.6 may be coupled using a reagent such as dicyclohexylcarbodiimide in an inert solvent. The product may then be treated with trifluoracetic acid to cleave the t-butyl ester. The product may then be treated with borane in a solvent such as tetrahydrofuran to reduce the carboxylic acid and give compound 16.4.

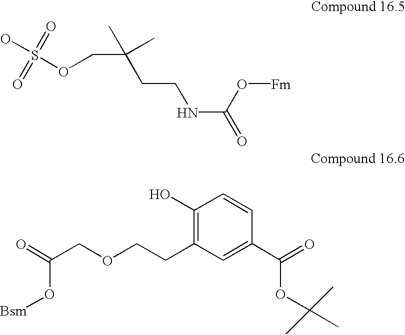

Compound 16.5 may be prepared by reacting 4-Amino-2,2-dimethyl-butan-1-ol with 9-fluorenylmethyl N-succinimidyl carbonate and then treating the product with sulfur trioxide-pyridine in an inert. The following references relate to this subject matter: Roberts J. C., et al., “Neopentyl Ester Protecting Groups for Arylsulfonic Acids,” Tetrahedron Letters, 38(3):355-358 (1997), the contents of which are incorporated herein by reference in their entirety.

Compound 16.6 may be prepared by a multi-step process. A Friedel-Crafts reaction between 4-hydroxy-benzoic acid and chlorocarbonylmethoxy acetic acid ethyl ester will give 3-(2-ethoxycarbonylmethoxy acetyl)-4-hydroxy-benzoic acid. Catalytic reduction with Pd on carbon will give 3-(2-Ethoxycarbonylmethoxyethyl)-4-hydroxy-benzoic acid. Treatment with acetic anhydride and base will give 4-acetoxy-3-(2-ethoxycarbonylmethoxy-ethyl)-benzoic acid. Treatment with di-t-butyl pyrocarbonate, t-butanol and dimethylaminopyridine in an inert solvent will give 4-acetoxy-3-(2-ethoxycarbonylmethoxy-ethyl)-benzoic acid tert-butyl ester. Treatment with sodium hydroxide, followed by HCl, will give 3-(2-Carboxymethoxy-ethyl)-4-hydroxy-benzoic acid tert-butyl ester. Treatment with trifluoroacetic anhydride and base will give 3-(2-Carboxymethoxy-ethyl)-4-(2,2,2-trifluoro-acetoxy)-benzoic acid tert-butyl ester. Coupling to (1,1-Dioxo-1H-1λ6-benzo[b]thiophen-2-yl)-methanol using a reagent such as dicyclohexylcarbodiimide will give 3-[2-(1,1-Dioxo-1H-1λ6-benzo[b]thiophen-2-ylmethoxycarbonylmethoxy)-ethyl]-4-(2,2,2-trifluoro -acetoxy)-benzoic acid tert-butyl ester. Hydrolysis of the trifluoroacetate ester will give compound 16.6.

### Example 17

Compound 17 is a multifunctional delivery vehicle which will target tumor cells that are positive for both α5β3 integrin, and MMPs 2, 3, 9, 12, and 13. The drug has a masked folic acid moiety as an intracellular transport ligand. The highly potent toxin 2-pyrrolinodoxorubicin will be liberated upon activation of an intracellular disulfide trigger. Cleavage of the disulfide by thiol reductases will unmask a thiol group, which will, via an intramolecular nucleophilic reaction, cleave the carbamate group and release the toxin. The following references relate to this subject matter: Batt D. G., et al., “Disubstituted Indazoles as Potent Antagonists of the Integrin αᵥβ3,” J Med chem, 43:41-58 (2000); Nagy A., et al., “High Yield Conversion of Doxorubicin to 2-pyrrolinodoxorubicin, an Analog 500-1000 Times More Potent: Structure-Activity Relationship of Daunosamine-Modified Derivatives of Doxorubicin,” Proc Natl Acad Sci USA, 93:2464-2469 (1996); WO99/25687, May 27, 1999, Williams R. A., et al., “Aromatic Sulfone Hydroxamic Acid Metalloprotease Inhibitor”; U.S. Pat. No. 5,932,595, Aug. 3, 1999, Bender et al., “Matrix Metalloprotease Inhibitors”; Lovejoy B., et al., “Crystal Structures of MMP-1 and -13 Reveal the Structural Basis for Selectivity of Collagenase Inhibitors,” Nat Struct Biol, 6(3):217-21 (1999); Botos I., et al., “Structure of Recombinant Mouse Collagenase-3 (MMP-13),” J Mol Biol, 292:837-844 (1999); Hutchins J. E. C.; Fife T. H., “Fast Intramolecular Nucleophilic Attack by Phenoxide Ion on Carbamate Ester Groups,” J Am Chem Soc, 95(7):2282-2286 (1973); Fife T. H., et al., “Highly Efficient Intramolecular Nucleophilic Reactions. The Cyclization of p-Nitrophenyl N-(2-Mercaptophenyl)-N-methylcarbamate and Phenyl N-(2-Aminophenyl)-N-methylcarbamate,” J Am Chem Soc, 97(20):5878-5882 (1975), the contents of which are incorporated herein by reference in their entirety.

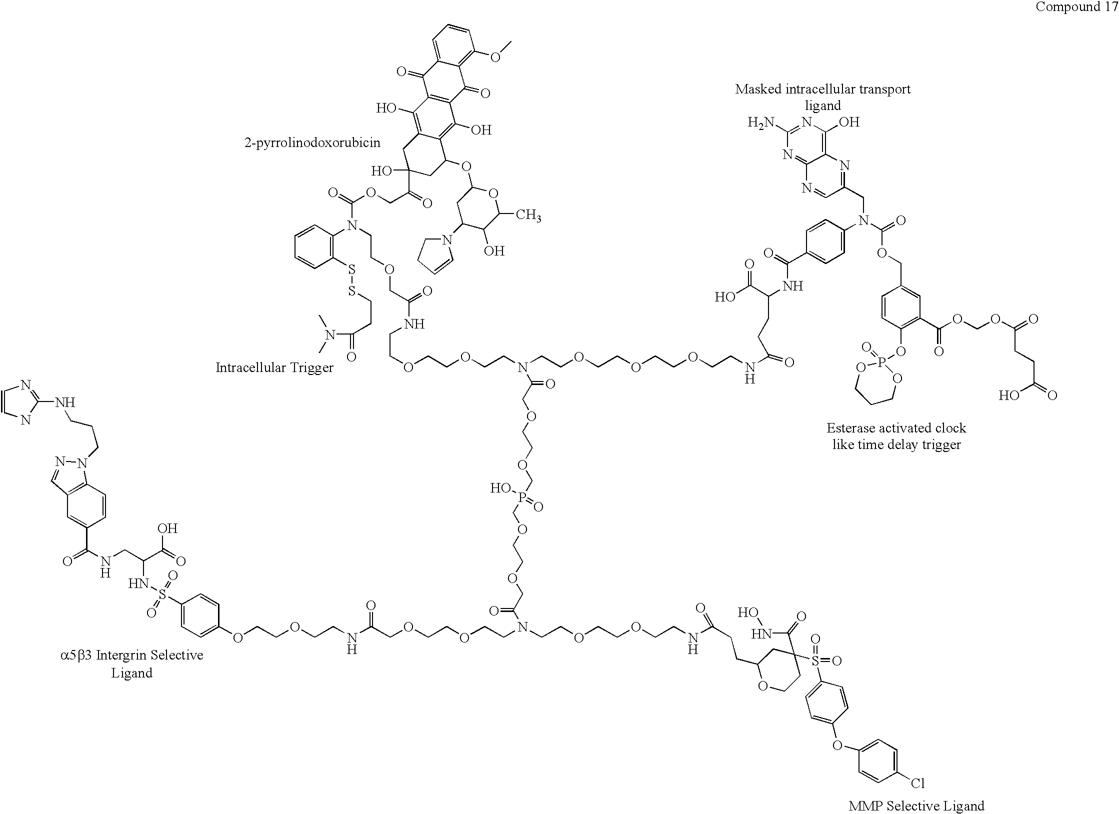

Compound 17 may be prepared by treating structure 17b with base to cleave the Fmoc and related fluorenylmethyl esters (Fm) groups.

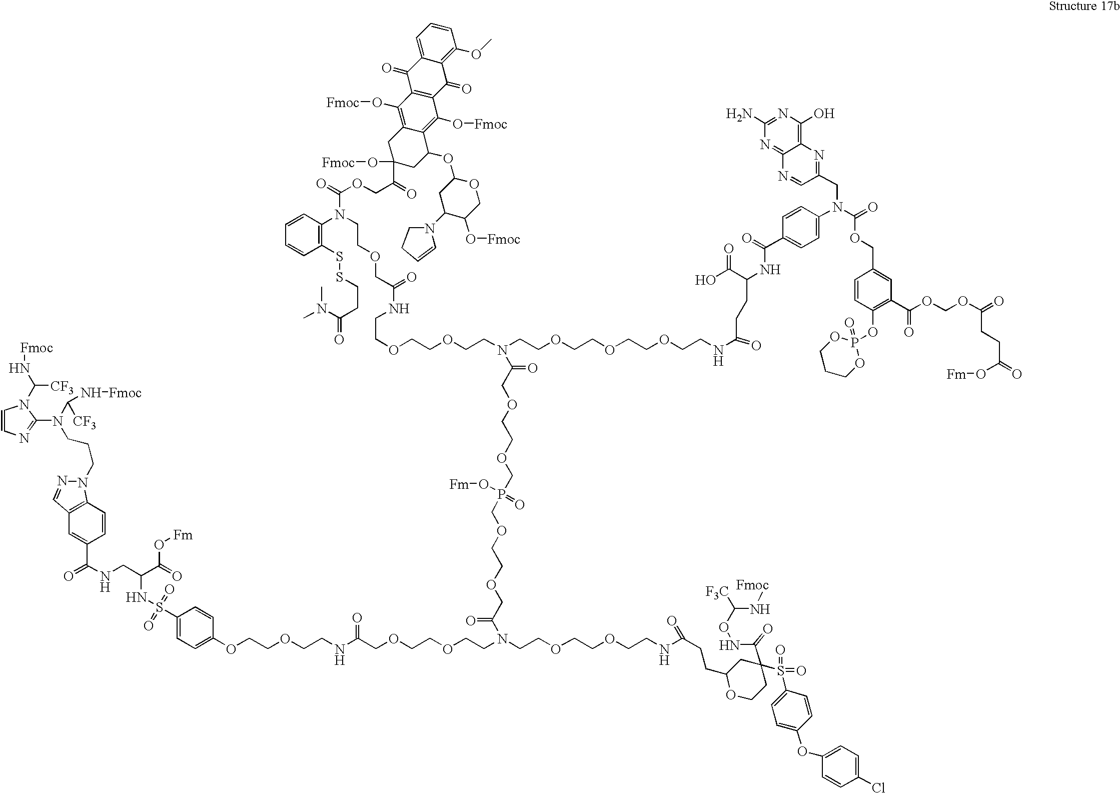

Compound 17b may be prepared by coupling compounds 17.1a and 17.2a.

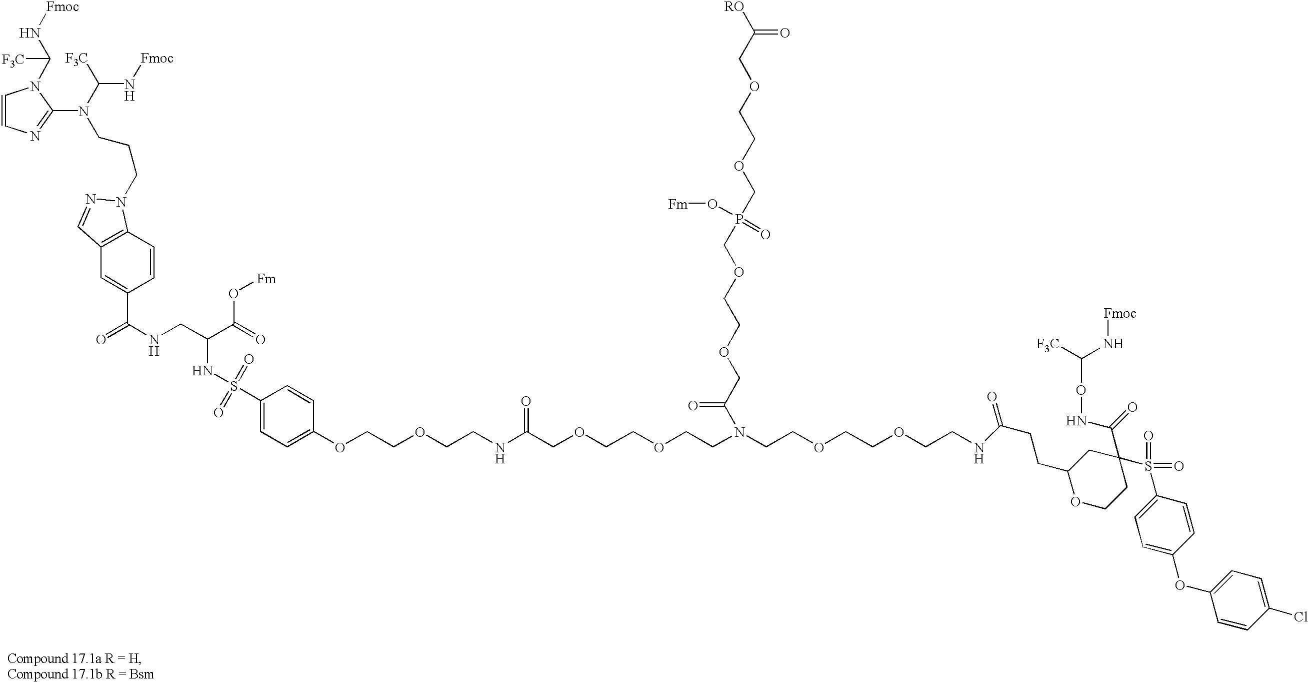

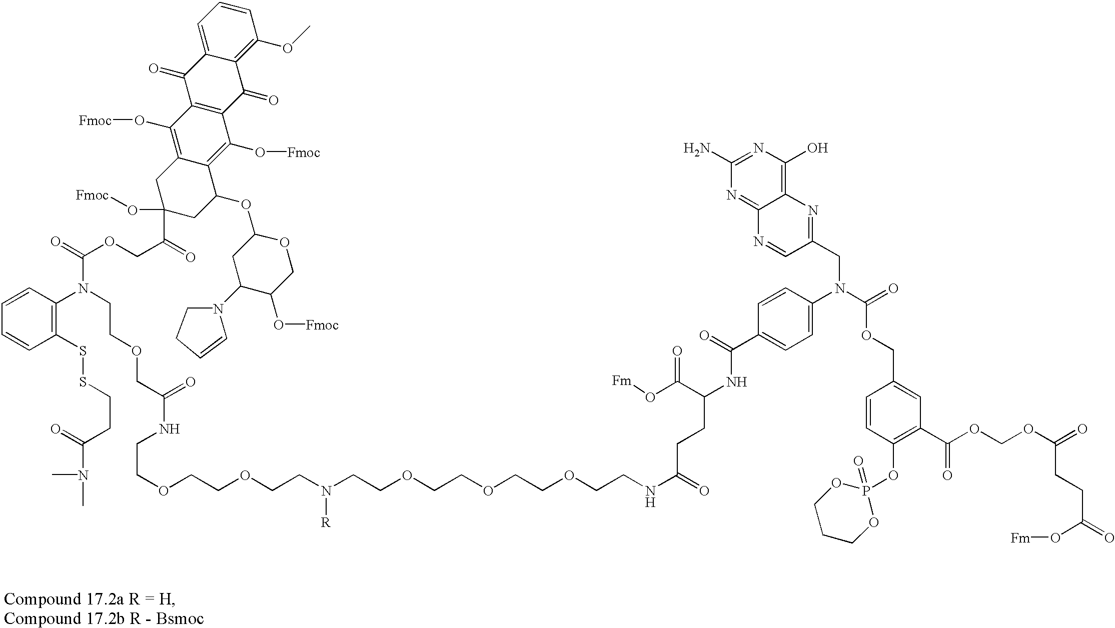

Compound 17.1a and 17.2a may be prepared by treatment of 17.1b and 17.2b with tris(2-aminoethyl)amine under conditions that will leave the Fmoc and Fm esters intact.

Compound 17.1b may be prepared in a multi-step procedure. Treating compound 17.4 with trifluoroacetic acid will selectively deblock the t-butyl ester group. The product may then be coupled to compound 17.5. Next, the 2,2,2 trichloroethoxycarbonyl protecting group may be selectively removed with Zn and acid. Then the product may be coupled to compound 17.6 to give compound 17.1b.

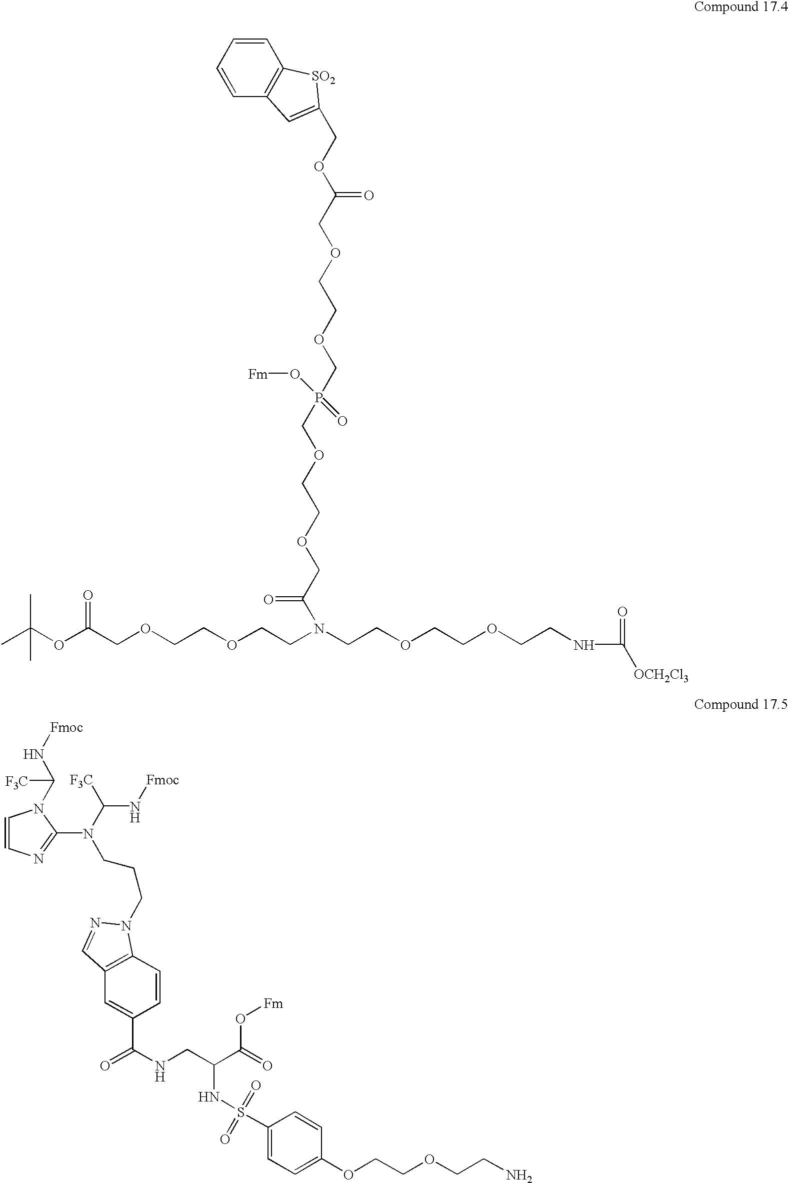

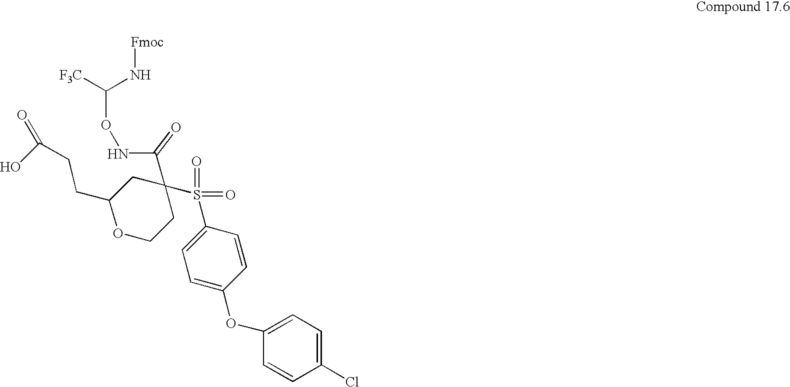

Compound 17.4 may be prepared by coupling compound 17.7 and 17.8.

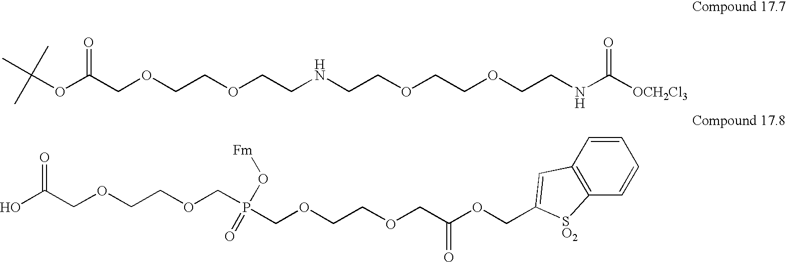

Compound 17.7 may be prepared by treating [2-(2-{2-[2-(2-Amino-ethoxy)-ethoxy]-ethylamino}-ethoxy)-ethoxy]-acetic acid tert-butyl ester (compound 17.7a) with 2,2,2,trichloroethyl N-succinimidyl carbonate in an inert solvent.

Compound 17.7a may be prepared by reacting [2-(2-Chloro-ethoxy)-ethoxy]-acetic acid tert-butyl ester with 2-{2-[2-(trityl-amino)-ethoxy]-ethoxy}-ethylamine in the presence of base in an inert solvent, isolating the product and then selectively removing the trityl group with acid. [2-(2-Chloro-ethoxy)-ethoxy]-acetic acid may be prepared by oxidizing 2-[2-(2-chloro-ethoxy)-ethoxy]-ethanol. This may be carried out by catalytic oxygenation with Pt on carbon or platinum dioxide in water with air. The t-butyl ester may be prepared using routine methods well known to one skilled in the arts. The following references relate to this subject matter: Tsou K. C., et al., “Synthesis of 5-Fluoro-2′-deoxyuridine-5′-carboxylic Acid and Its Derivatives,” J Med Chem, p.173 (1969), the contents of which are incorporated herein by reference in their entirety.

2-{2-[2-(trityl-amino)-ethoxy]-ethoxy}-ethylamine may be made by treating trityl chloride with amino)-ethoxy]-ethoxy}-ethylamine and isolating the monosubstituted product.

Compound 17.8 may be prepared in a multistep synthesis. Compound 17.8.1a may be prepared by reacting one equivalent of (2-Chloromethoxy-ethoxy)-acetic acid methyl ester and bis(trimethylsilyl)phosphonite, silylating the product, and without isolation, reacting with an additional equivalent of (2-Chloromethoxyethoxy)-acetic acid methyl ester. The following references relate to this subject matter: Boyd E. A.; Regan A. C., “Synthesis of Alkyl Phosphinic Acids from Silyl Phosphonites and Alkyl Halides,” Tetrahedron Letters, 35(24):4223-4226 (1994), the contents of which are incorporated herein by reference in their entirety.

(2-Chloromethoxy-ethoxy)-acetic acid methyl ester may be prepared by chloromethylation of 2-Hydroxy-ethoxy)-acetic acid methyl ester with HCL and paraformaldehyde.

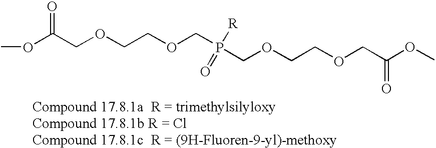

Compound 17.8.1a may be treated with thionyl chloride to give compound 17.8.1b. Reaction with 9-H-fluorenyl-9-yl-methanol and a base, such as triethylamine, will give compound 17.8.1c. Hydrolysis of the methyl esters by esterase or with a catalyst such as distannoxane, followed by coupling of 1 equivalent of 1,1-Dioxobenzo[b]thiophene-2-yl-methanol, with dicyclohexylcarbodiimide will give compound 17.8 after purification.

Compound 17.2b may be prepared in a multi-step procedure.

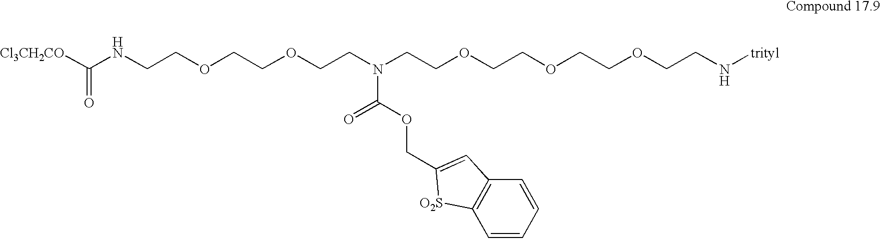

Treating compound 17.9 with acid will remove the trityl group. The product may then be coupled with compound 6.2.0b. The trichloroethoxycarbonyl group may then be removed with Zn and acid. The product may then be coupled to compound 17.11 to give compound 17.2b.

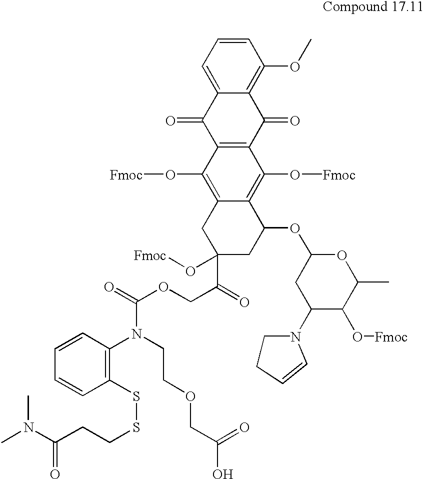

Compound 17.11 may be prepared by reacting compound 17.11.1 and compound 17.11.2 in an inert solvent in the presence of a base, such as pyridine, and then cleaving the Bsm ester with tris(2-aminoethyl)amine under conditions that will leave the Fmoc groups intact.

In an alternate method, compound 17.11.1 may be treated with phosgene in a solvent, such as toluene at low temperature to generate the carbamoyl chloride derivative, which may then be reacted with 2-pyrrolinodoxirubicin in the presence of a base such as pyridine. In this case, the Fmoc protection of the 2-pyrrolinodoxirubicin need not be employed.

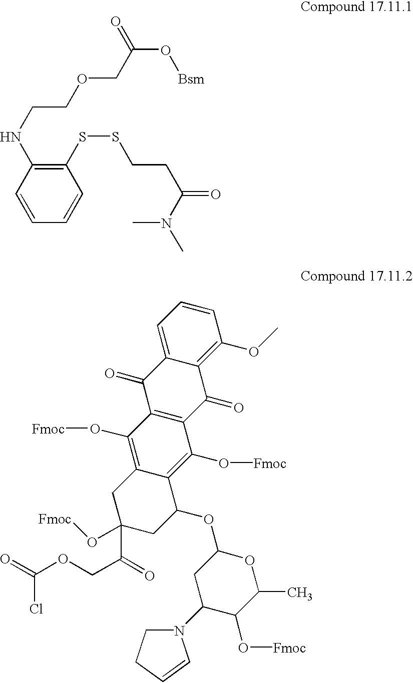

Compound 17.11.1 may be prepared by a multi-step process. Reacting diethyl azidocarboxylate with and 3-mercapto-N,N-dimethyl-propionamide and then reacting the product with compound 17.11.1a will form the mixed disulfide compound 17.11.1b. The following references relate to this subject matter: Mukaiyama T.; Takahashi K., “A Convenient Method for the Preparation of Unsymmetrical Disulfides by the use of Diethyl Azodicarboxylate,” Tetrahedron Letters, 56:5907-5908 (1968), the contents of which are incorporated herein by reference in their entirety.

Treating compound 17.11.1b with di-t-butyl pyrocarbonate and in an inert solvent will give compound 17.11.1c. Treating with (1,1-Dioxo-1H-1λ6-benzo[b]thiophen-2-yl)-methanol and a reagent such as dicyclohexylcarbodiimide in an inert solvent, followed by acid treatment to cleave the t-Boc group, will give compound 17.11.1d.

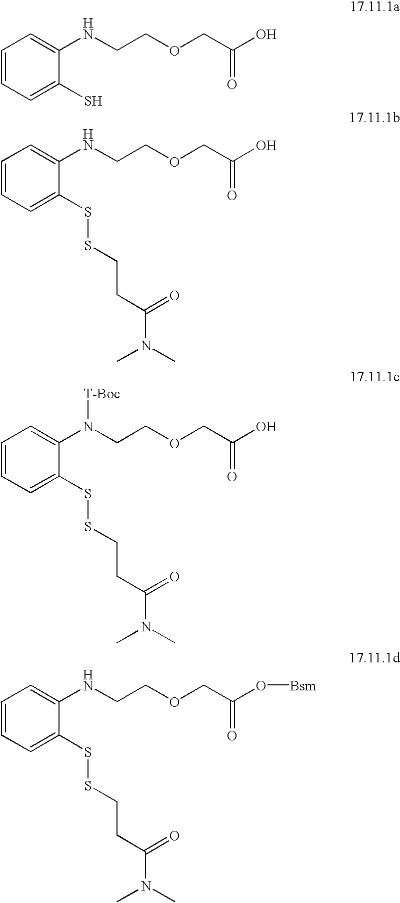

Compound 17.11.1a may be prepared by a multi-step procedure. 2-Aminophenyl-disulfide may be reacted with (2-Oxo-ethoxy)-acetic acid methyl ester in the presence of a dehydrating agent to form the Schiff base. The imine may then be reduced with a reagent such as sodium borohydride to give compound 17.11.1a.

The compound (2-Oxo-ethoxy)-acetic acid methyl ester may be prepared in a multi-step procedure. Treating [1,4]Dioxane-2,6-dione with methanol and dimethylaminopyridine will give methoxycarbonylmethoxy-acetic acid. This may then be converted into chlorocarbonylmethoxy-acetic acid methyl ester by treatment with thionyl chloride. The acid chloride may then be reduced to the desired aldehyde with lithium tri-tert-butoxyaluminum hydride at low temperature in an inert solvent.

Compound 17.11.2 may be prepared by a multi-step process. Treating 2-pyrrolinodoxorubicin in an inert solvent with 1 equivalent of 1,1-dioxobenzo[b]thiophene-2-yl-methoxycarbonyl chloride and a base such as pyridine will protect the primary hydroxy group on C14. The product may then be treated with 4 equivalents of 9-H-fluorenyl-9-yl-methoxycarbonyl chloride and a base such as pyridine. The product may then be treated with tris(2-aminoethyl)amine to selectively remove the 1,1-dioxobenzo[b]thiophene-2-yl-methoxycarbonyl protecting group. The product may then be treated with phosgene to give the chloroformate (compound 17.11.2).

Compound 17.5 may be prepared by coupling compound 17.5.1 and compound 17.5.2 and then removing the 1,1-dioxobenzo[b]thiophene-2-yl-methoxycarbonyl protecting group with tris(2-aminoethyl)amine.

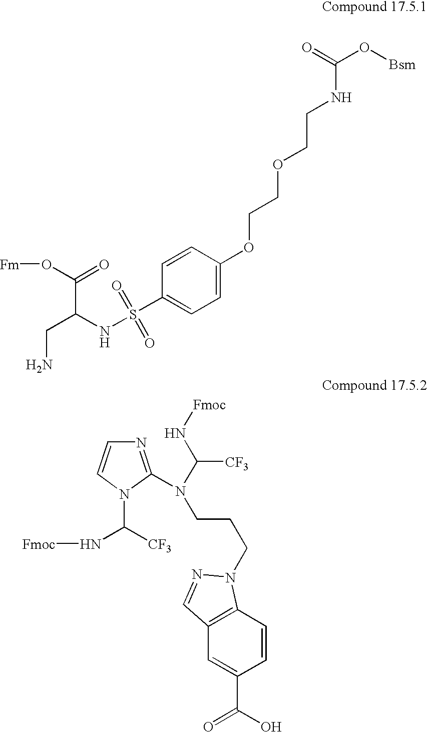

Compound 17.5.1 may be made by reacting compound 17.5.1a and 17.5.1b, in an inert solvent, in the presence of a base such as pyridine and then removing the t-Boc group with trifluoracetic acid.

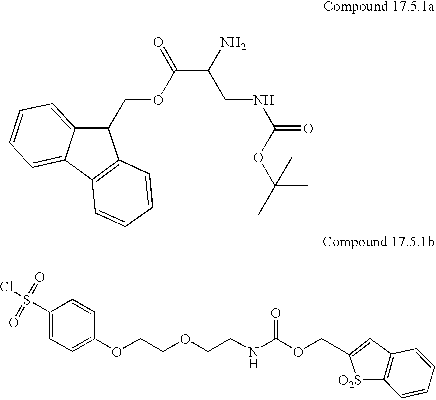

Compound 17.5.1a may be prepared from (S) 3-Amino-2-benzyloxycarbonylamino-propionic acid. Treatment with di-t-butyl dicarbonate will give 2-benzyloxycarbonylamino-3-tert-butoxycarbonylamino-propionic acid. Catalytic hydrogenation, followed by treatment with 2,2,2 trichloroethyl chloroformate and base, will give 3-tert-Butoxycarbonylamino-2-(2,2,2-trichloroethoxycarbonylamino)-propionic acid. Coupling with (9H-Fluoren-9-yl)-methanol will give 3-tert-Butoxycarbonylamino-2-(2,2,2-trichloro-ethoxycarbonylamino)-propionic acid 9H-fluoren-9-yl methyl ester. Treatment with Zn and acid will remove the trichloroethylcarbonyl protecting group and give compound 17.5.1a.

Compound 17.5.1b may be prepared by treating compound 17.5.1c with a reagent such as thionyl chloride.

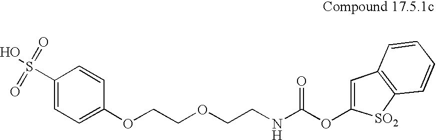

Compound 17.5.1c may be prepared by a multi-step procedure. Reacting 4-hydroxy-benzenesulfonic acid and 1-(2-Azido-ethoxy)-2-chloro-ethane in the presence of base will give 4-[2-(2-azido-ethoxy)-ethoxy]-benzenesulfonic acid. Reduction of the azido group by catalytic hydrogenation will give 4-[2-(2-amino-ethoxy)-ethoxy]-benzenesulfonic acid. Treatment with 1,1-dioxobenzo[b]thiophene-2-yl-methoxycarbonyl chloride and a base such as pyridine will give compound 17.5.1c.

Compound 17.5.2 may be prepared by treating 1-[3-(1H-Imidazol-2-ylamino)-propyl]-1H-indazole-5-carboxylic acid (compound 17.5.2a) with 2 equivalents of (1-Chloro-2,2,2-trifluoro-ethyl)-carbamic acid 9H-fluoren-9-ylmethyl ester (compound 17.5.2b) and base in an inert solvent. Compound 17.5.2b may be prepared by reacting carbamic acid 9H-fluoren-9-ylmethyl ester with trifluoroacetadehyde and then treating with phosphorous trichloride in an inert solvent.

The following references relate to this subject matter: Batt D. G., et al., “Disubstituted Indazoles as Potent Antagonists of the Integrin αᵥ3,” J Med Chem, 43:41-58 (2000); Weygand F., et al., “2,2,2-Trifluoro-1-acylaminoethyl Groups as Protective Groups for Imino Groups of Histidine in Peptide Synthesis,” Chem Ber, 100(12):3841-9 (1967); Weygand, Friedrich; Steglich, Wolfgang; Pietta, Pier G., Chem Ber, 99: p.1944 (1966), the contents of which are incorporated herein by reference in their entirety.

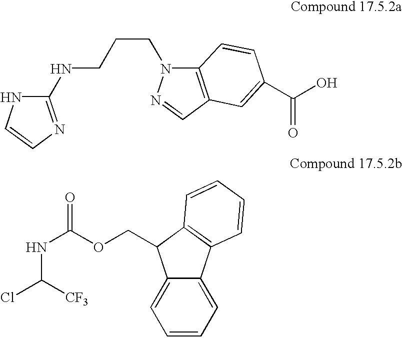

Compound 17.6 may be prepared by treating compound 17.6.1a with acid to remove the tert-butoxy group. Alternatively, esterase may be employed.

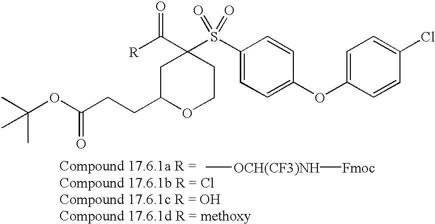

Compound 17.6.1a may be prepared by treating compound 17.6.1b with 0-trimethylsilyl protected hydroxylamine and base in an inert solvent, followed by hydrolysis of the silyl protecting group, followed by treatment with (1-Chloro-2,2,2-trifluoro-ethyl)-carbamic acid 9H-fluoren-9-ylmethyl ester and base.

Alternate synthetic approaches would be to react the hydroxamate with 4,4′-dimethoxytrityl chloride or 4-methoxytritylchloride, or pixyl chloride instead of (1-Chloro-2,2,2-trifluoro-ethyl)-carbamic acid 9H-fluoren-9-ylmethyl ester. These protecting groups may be removed at the end of the synthesis with dilute acid under conditions that do not cleave the acetal of the doxorubicin group.

Compound 17.6.1.b may be prepared by the treatment of the carboxylic acid derivative 17.6.1c with well known agents for the synthesis of acid chlorides such as triphenylphosphine/carbon tetrachloride, or thionyl chloride, or oxalyl chloride and dimethylformamide in inert solvents.

Compound 17.6.1c may be prepared by the selective hydrolysis of the methyl ester in compound 17.6.1d with aqueous sodium hydroxide.

Compound 17.6.1d may be prepared by the alkylation of compound 17.6.2 with compound 17.6.3 using a base such as sodium hydride in an inert solvent. The following reference relates to this subject matter: WO99/25687, May 27, 1999, Williams R. A., et al., “Aromatic Sulfone Hydroxamic Acid Metalloprotease Inhibitor”, the contents of which is incorporated herein by reference in its entirety.

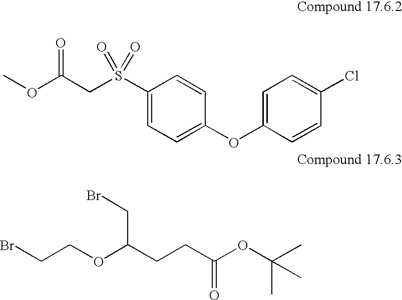

Compound 17.6.3 may be prepared by a multi-step procedure. Treating 5-hydroxy-tetrahydro-pyran-2-one with one equivalent of ethylene oxide in the presence of a strong base such as potassium tert-butoxide in an inert solvent will give, after purification by chromatography, 5-(2-Hydroxy-ethoxy)-tetrahydropyran-2-one. Treatment with HBr, followed by trimethylbromosilane in an inert solvent, will give 5-bromo-4-(2-bromo-ethoxy)-pentanoic acid after hydrolysis of the silyl ester. The tert-butyl ester (compound 17.6.3) may then be prepared by treatment with an excess of iso-butylene, catalyzed with strong acids, such as para-toluenesulfonic acid.

### Example 18

Compound 18 is similar to compound 17 in its targeting specificity. The difference is in the MMP selective ligand.

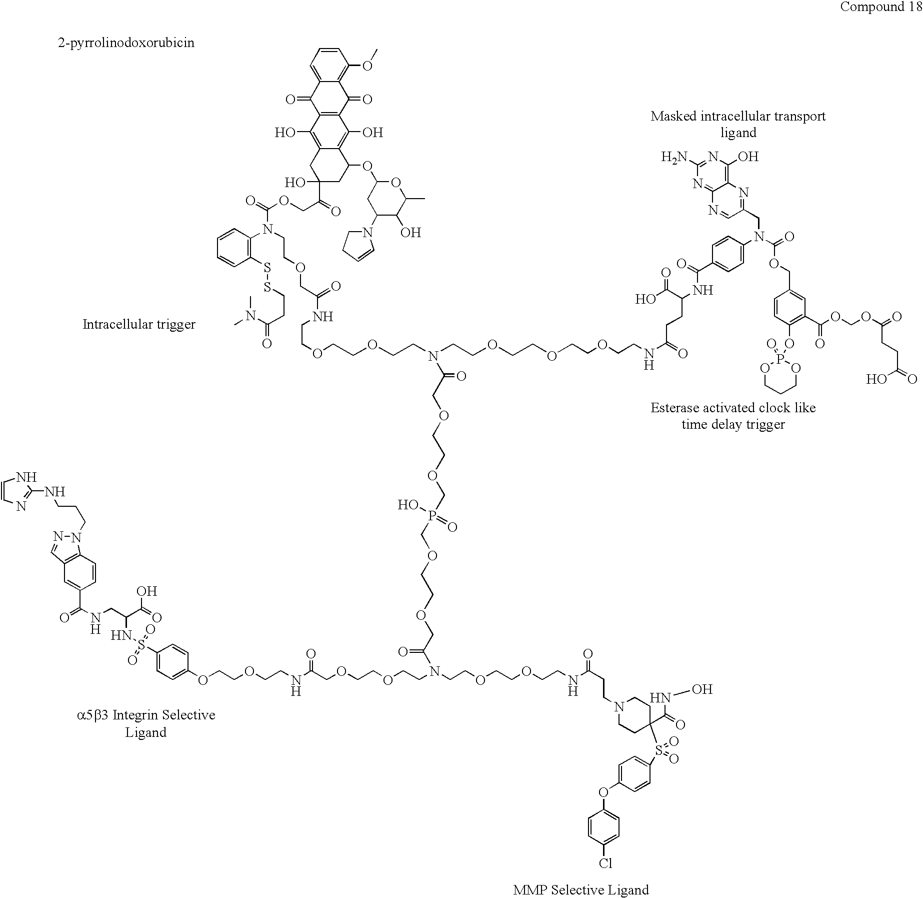

Compound 18 may be prepared using the procedures as described for compound 17 by replacing compound 17.6 with compound 18.1.

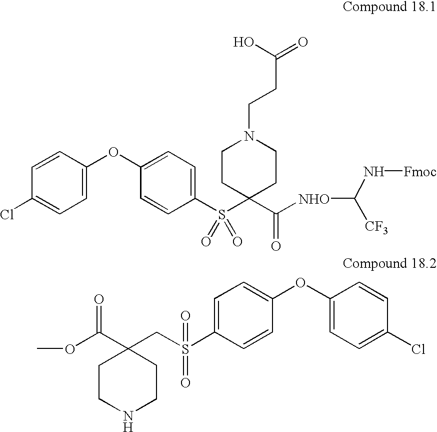

Compound 18.1 may be prepared by a multi-step procedure. Alkylating compound 18.2 with t-butyl 3-bromopropanoate in the presence of base, in an inert solvent, will give compound 18.3. The following references relate to this subject matter: EPO 780 386 A1 Jun. 25, 1997 Bender S. L., “Matrix Metalloprotease Inhibitors”, the contents of which are incorporated herein by reference in their entirety.

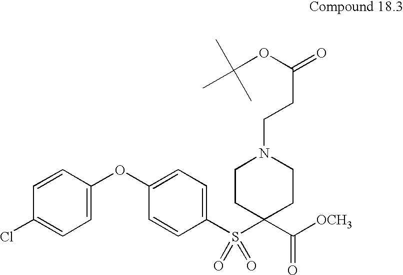

Compound 18.3 may be transformed into compound 18.1 using the same reaction procedures described to transform compound 17.6.1d into compound 17.6.

### Example 19

Compound 19 is similar to compound 18, however a different α5β3 integrin selective ligand is employed. The following references relate to this subject matter: WO 96-US13500 1997 Ruminski P. G., et al., “Preparation of Meta-Guanidine, Urea, Thiourea or Azacyclic Amino Benzoic Acid Derivatives as Integrin Antagonists”; Carron C. P., et al., “A Peptidomimetic Antagonist of the Integrin αvβ3 Inhibits Leydig Cell Tumor Growth and the Development of Hypercalcemia of Malignancy,” Cancer Res, 58(9):1930-1935 (1998), the contents of which are incorporated herein by reference in their entirety.

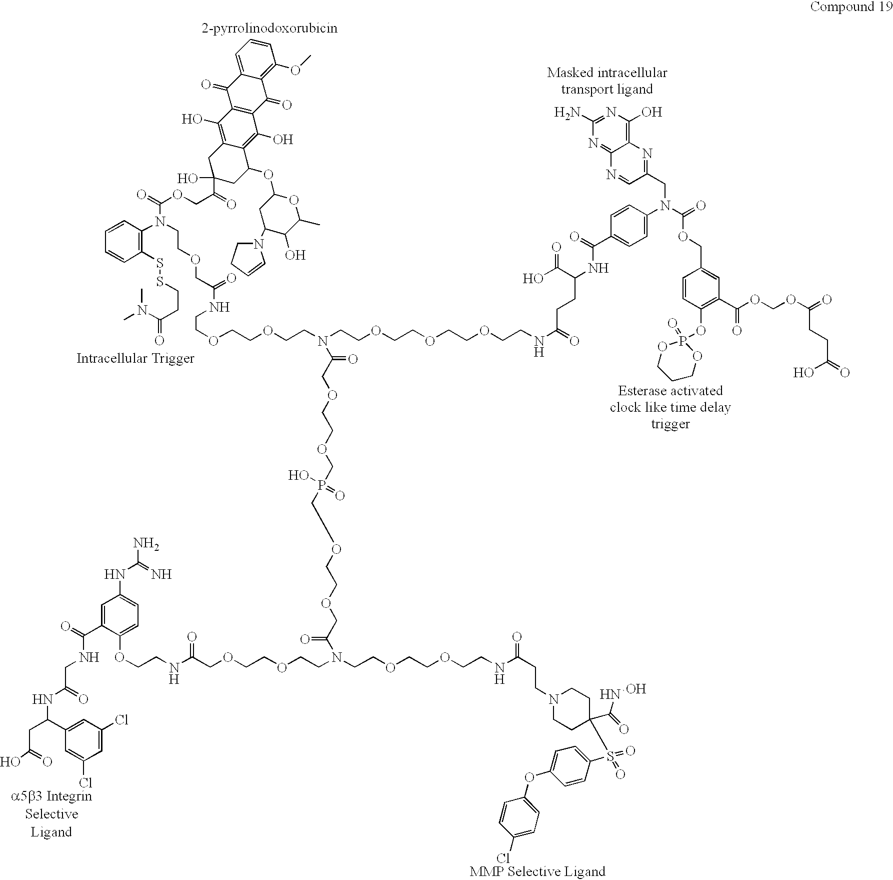

Compound 19 may be prepared using the procedures described for compounds 17 and compounds 18 by substituting compound 19.1a for compound 17.5.

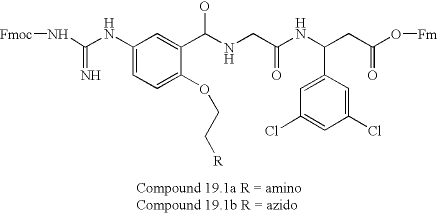

Compound 19.1a may be prepared by the reduction of the azido compound 19.1b with triphenylphosphine and water in an inert solvent. The following references relate to this subject matter: Pak J. K.; Hesse M., “Synthesis of Penta-N-Protected Homocaldopentamine and Its Selective Acylation,” J Org Chem, 63:8200-8204 (1998), the contents of which are incorporated herein by reference in their entirety.

Compound 19.1b may be prepared by coupling compound 19.2 and compound 19.3.

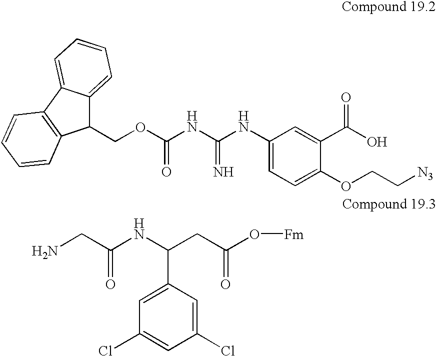

Compound 19.2 may be prepared by treating compound 19.4 with 9H-fluoren-9-ylmethyl chloroformate and a base such as pyridine and catalytic amounts of dimethylaminopyridine, and then treating with acid to remove the t-Boc and t-butyl ester groups. Compound 19.4 may be prepared by the selective removal of the p-methoxybenzyloxycarbonyl protecting group from compound 19.5 with dilute trifluoroacetic acid in an inert solvent. The following references relate to this subject matter: Wang S. S., et al., “4-Methoxybenzyloxycarbonyl Amino Acids in Solid Phase Peptide Synthesis,” Int J Peptide Protein Res, 30:662-667 (1987), the contents of which are incorporated herein by reference in their entirety.

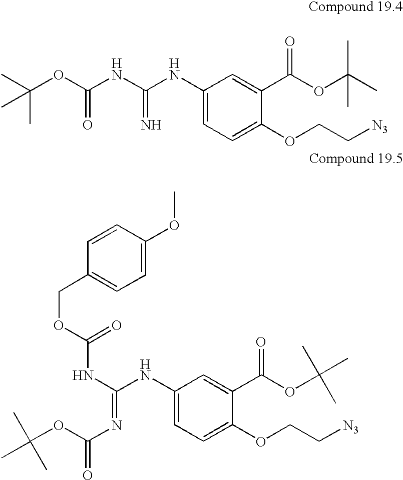

Compound 19.5 may be prepared by reacting compound 19.6 and compound 19.7 in the presence of a base such as triethylamine in an inert solvent.

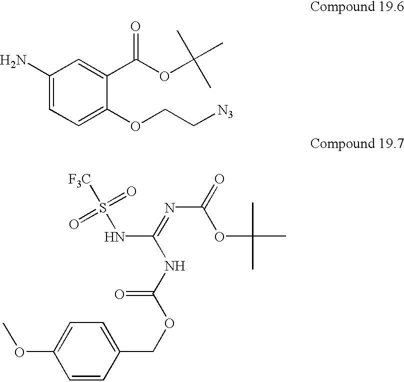

Compound 19.7 may be prepared by a multi-step procedure. Treating guanidine hydrochloride with base and one equivalent of a reagent such as 2-(4-methoxybenzyloxycarbonyloxyimino)-2-phenyl acetonitrile, followed by additional base and 1 equivalent of di-t-butyl dicarbonate, followed by treatment with NaH and triflic anhydride in an inert solvent will give compound 19.7. The following references relate to this subject matter: Feichtinger K., et al., “Diprotected Triflylguanidines: A New Class of Guanidinylation Reagents,” J Org Chem, 63:3804-3805 (1998), the contents of which are incorporated herein by reference in their entirety.

Compound 19.6 may be prepared by a multi-step procedure. The compound 2-Hydroxy-5-nitro-benzoic acid may be treated with pivaloyl chloride and a base in an inert solvent to give 2-(2,2-Dimethyl-propionyloxy)-5-nitro-benzoic acid.

Treatment with isobutylene and an acid will give 2-(2,2-dimethyl-propionyloxy)-5-nitro-benzoic acid tert-butyl ester. Treatment with aqueous sodium hydroxide will give, after neutralization, 2-hydroxy-5-nitro-benzoic acid tert-butyl ester. Treatment with one equivalent of a strong base and one equivalent of ethylene oxide in an inert solvent will give 2-(2-Hydroxy-ethoxy)-5-nitro-benzoic acid tert-butyl ester. Reduction of the nitro group by catalytic hydrogenation with Pd catalysis will give 5-Amino-2-(2-hydroxy-ethoxy)-benzoic acid tert-butyl ester. Treatment with 2-(4-methoxybenzyloxycarbonyloxyimino)-2-phenyl acetonitrile and base will give compound 19.6a. Treatment with tosyl chloride in an inert solvent with a base such as pyridine will give Compound 19.6b. Treatment with lithium azide in an inert solvent such as dimethylformamide will give compound 19.6c. The selective removal of the p-methoxycarbonyl protecting group with 10% trifluoroacetic acid in methylene chloride will give compound 19.6.

Compound 19.3 may be prepared by a multi-step procedure. Treating 3-amino-3-(3,5-dichloro-phenyl)-propionic acid with di-t-butyl dicarbonate and then coupling with (9H-Fluoren-9-yl)-methanol and dicyclohexylcarbodiimide, followed by deprotection of the amino group with trifluoracetic acid will give 3-Amino-3-(3,5-dichloro-phenyl)-propionic acid 9H-fluoren-9-ylmethyl ester. Coupling with t-Boc glycine followed by acid treatment to remove the t-Boc group will give compound 19.3.

### Example 20

Compound 20 is a multifunctional drug delivery vehicle that will target tumors that jointly express matrilysin (or MMP's MMP1, 2, and 3) and plasmin or urokinase. The MMP ligand will bind to MMP7 with a Ki in the low nanomolar range. The plasmin ligand will acylate the active site of the serine protease resulting in essentially irreversible binding. The masked intracellular transporter ligand will bind to the folate receptor after triggering by phosphatase and transport the drug into the cell. The intracellular transport ligand employed is a potent inhibitor of glycinamide ribonucleotide transformylase and will be freed, from the remainder of the drug along with an immucillinGp analog, upon activation of a disulfide trigger by intracellular thioreductases. The liberated N-{2-[2-(2-Amino-ethoxy)-ethoxy]-ethyl}-amide derivative will inhibit glycinamide ribonucleotide transformylase and inhibit denovo purine synthesis. Published crystallography data indicate that the gamma carboxylate group is exposed to solvent. Accordingly, the attached linker should not compromise inhibitor affinity. The immucillinGP analog will inhibit hypoxanthene-guanine phosphoribosyltransferase and block the purine salvage pathway. The combination of inhibitors for both denovo and salvage pathways of purine metabolism should exert pronounced synergistic toxicity. The multifunctional drug delivery vehicle has a second intracellular trigger, which when activated by thioreductasae, will free doxorubicin, coupled to an intracellular targeting ligand, that will bind with high affinity to the peripheral benzodiazepam receptors located on mitochondria and impair drug efflux from the cell. Free radical processes initiated by doxorubicin, bound to the mitochondrial membranes, will damage the mitochondria resulting cytochrome release and apotopsis. Accordingly, this multifunctional delivery vehicle will provide multiple independent mechanisms of cytotoxicity. The following references relate to this subject matter: Varney M. D., et al., “Protein Structure-Based Design, Synthesis, and Biological Evaluation of 5-Thia-2,6-diamino-4(3H)-oxopyrimidines: Potent Inhibitors of Glycinamide Ribonucleotide Transformylase with Potent Cell Growth Inhibition,” J Med Chem, 40:2502-2524 (1997); Pratt L. M., et al., “The Synthesis of Novel Matrix Metalloproteinase Inhibitors Employing the Ireland-Claisen Rearrangement,” Bioorg Med Chem Lett, 8:1359-1364 (1998); Kozikowski A. P., et al., “Synthesis and Biology of a 7-Nitro-2,1,3-Benzoxadiazol-4-Yl Derivative of 2-Phenylindole-3-Acetamide: A Fluorescent Probe for the Peripheral-Type Benzodiazepine Receptor,” J Med Chem, 40(16):2435-9 (1997); Shi W., et al., “The 2.0 A Structure of Human Hypoxanthine-guanine Phosphoribosyltransferase in Complex with a Transition-state Analog Inhibitor,” Nature Structural Biology, 6(6):588-593 (1999); U.S. Pat. No. 6,066,722 May 23, 2000 Furneaux et al., “Inhibitors of Nucleoside Metabolism”, the contents of which are incorporated herein by reference in their entirety.

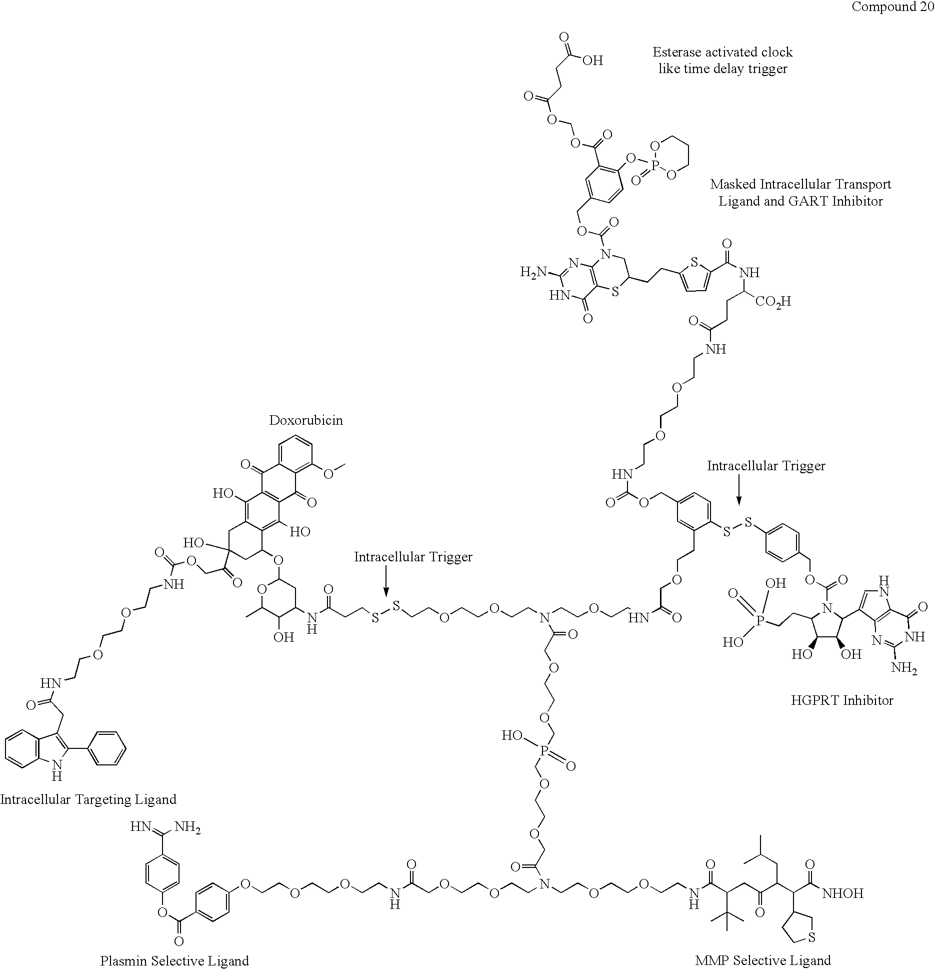

Compound 20 may be prepared by the deprotection of compound 20.1 with dilute acid to remove pixyl group followed by treatment with base to remove the Fmoc and fluorenylmethyl ester groups.

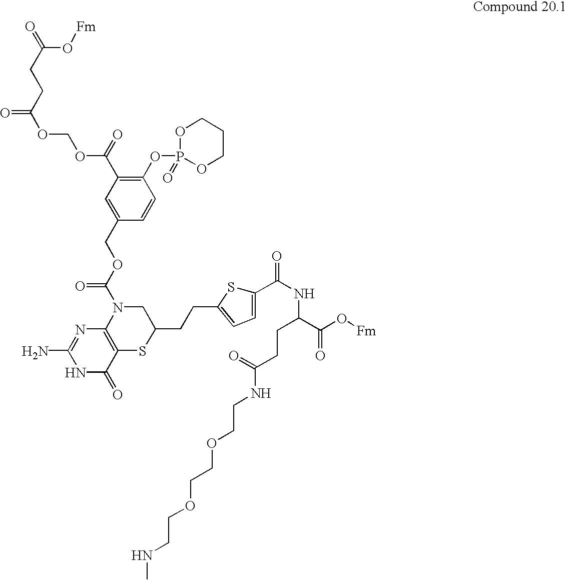

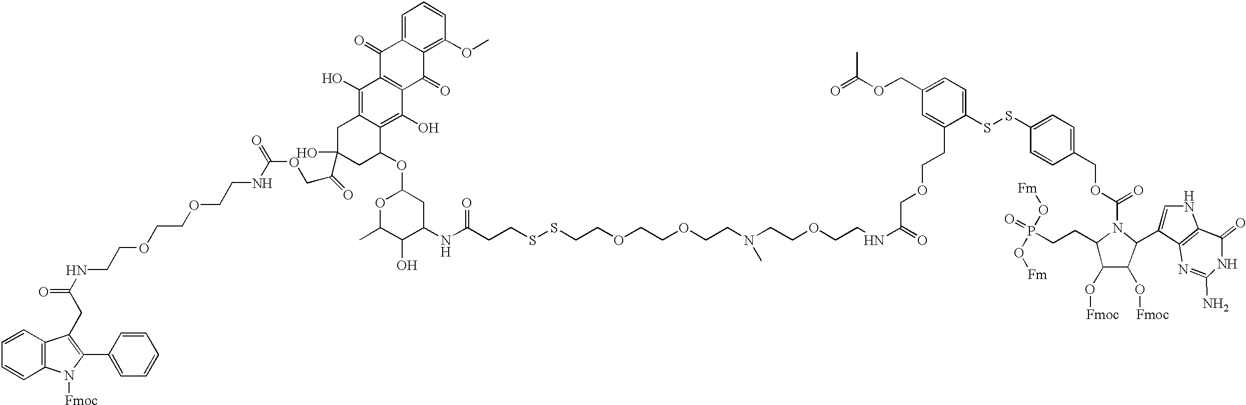

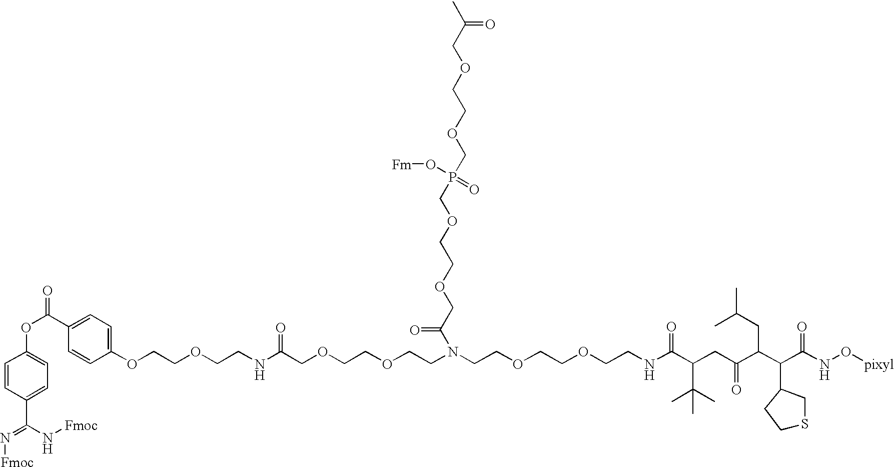

Compound 20.1 may be prepared by coupling compound 20.2a and 20.2b.

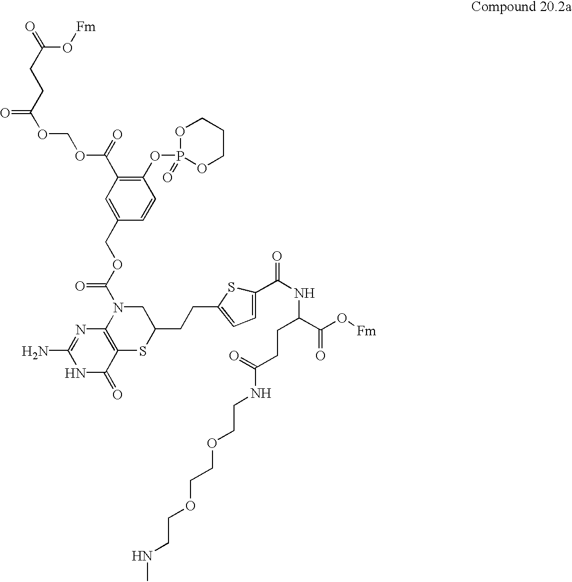

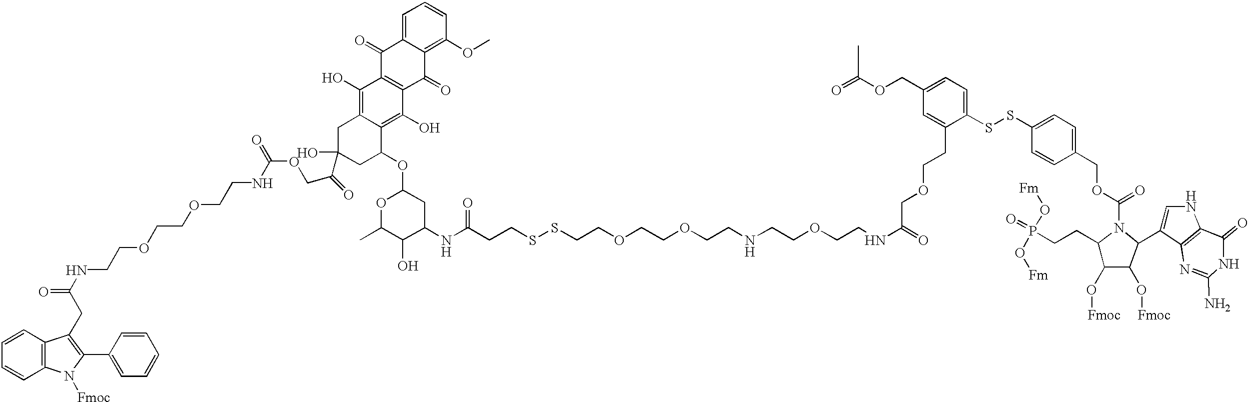

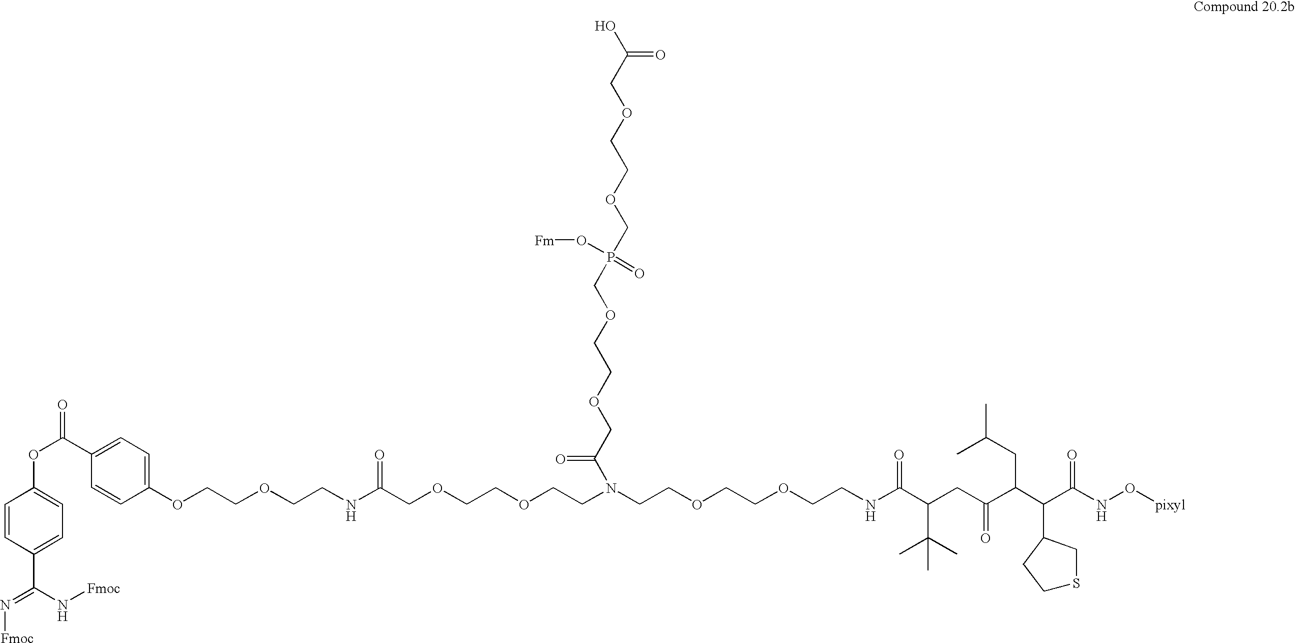

Compound 20.2a may be prepared by coupling compound 20.3a and 20.3b and then selectively removing the Bsmoc protecting group with tris(2-aminoethyl)amine under conditions that will leave the Fmoc and Fm esters intact.

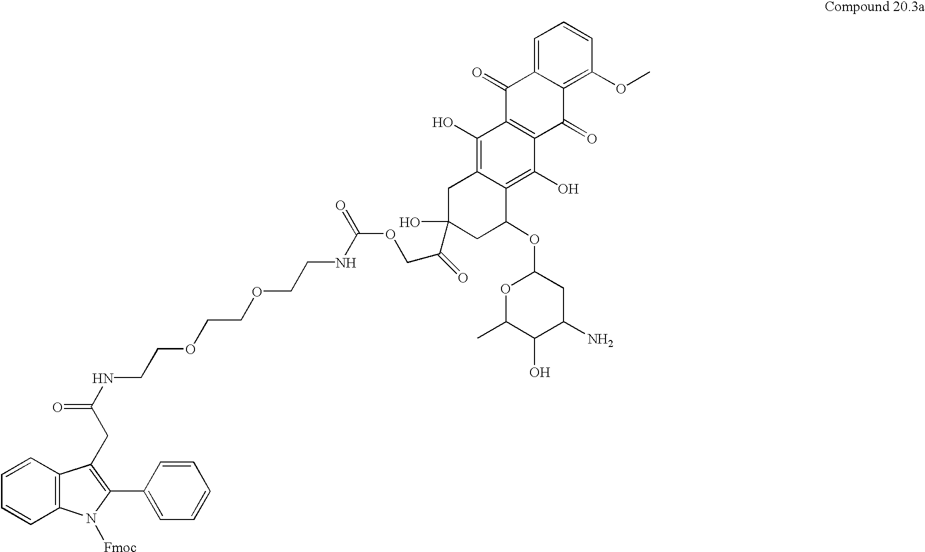

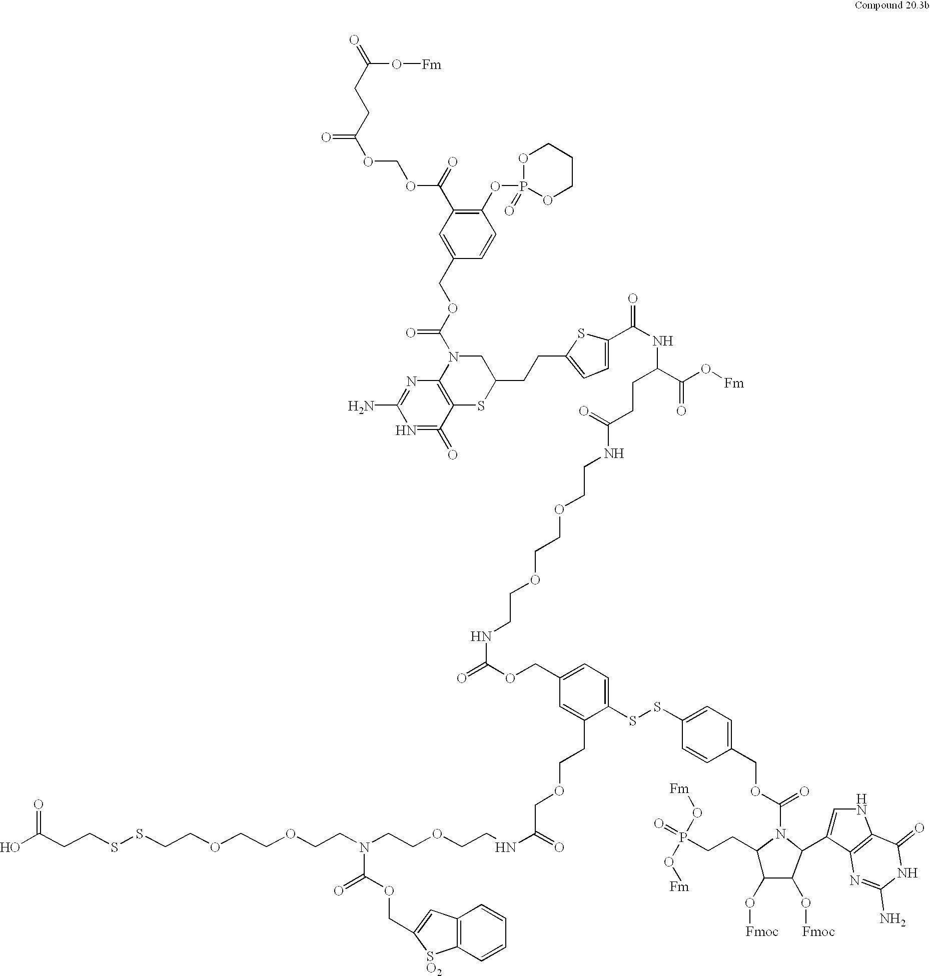

Compound 20.3a may be prepared by coupling compound 20.3a.1 and 20.3a.2 in an inert solvent in the presence of base (or a catalyst such as distannoxane) and then selectively removing the Bsmoc protecting group with tris(2-aminoethyl)amine under conditions that will leave the Fmoc group intact. The following references relate to this subject matter: Otera J., et al., “Distannoxane-Catalyzed Conversion of Chiral Alcohol to N-[1-(1-Naphthyl)ethyl]carbamate,” Synlett, 433-434 (1995), the contents of which are incorporated herein by reference in their entirety.

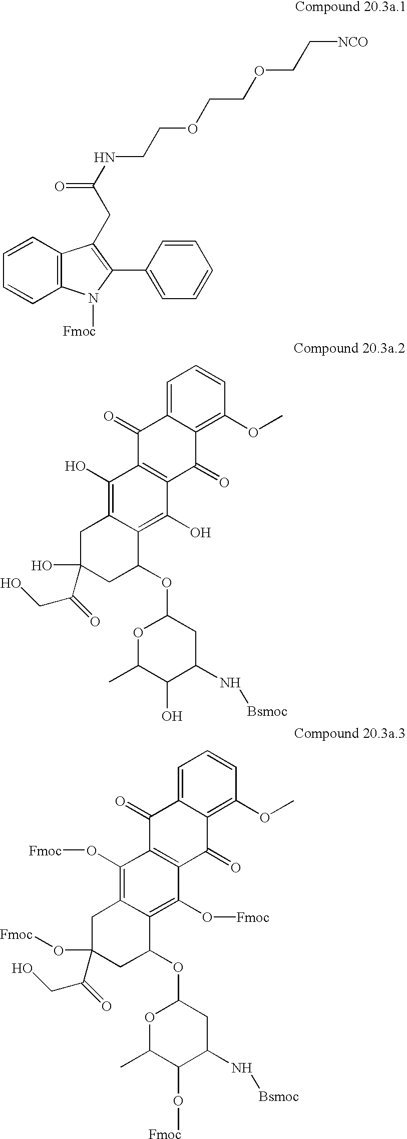

Compound 20.3a.1 Compound 20.3a.2 Compound 20.3a.3 In an alternate synthetic approach, compound 20.3a.1 may be reacted with compound 20.3a.3. Compound 20.3a.3 may be prepared by treating compound 20.3a.2 with trityl chloride and base in an inert solvent and then treating the product with 9-fluorenylmethyl chloroformate in the presence of base in an inert solvent, and then treating with acid to remove the trityl group.

Compound 20.3a.1 may be prepared by coupling compound 20.4a and 2-{2-[2-(Trityl-amino)-ethoxy]-ethoxy}-ethylamine, treating with acid to remove the trityl group, and then treating the product with phosgene in an insert solvent.

Compound 20.4a may be prepared by treatment of (2-Phenyl-1H-indol-3-yl)-acetic acid with 9-fluorenylmethyl chloroformate in the presence of base. The following references relate to this subject matter: Kozikowski A. P., et al., “Synthesis and Biology of a 7-Nitro-2,1,3-Benzoxadiazol-4-Yl Derivative of 2-Phenylindole-3-Acetamide: A Fluorescent Probe for the Peripheral-Type Benzodiazepine Receptor,” J Med Chem, 40(16):2435-9 (1997), the contents of which are incorporated herein by reference in their entirety.

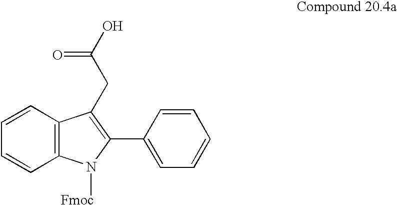

Compound 20.3b may be prepared by converting compound 20.6 into an active ester by treatment with a reagent such as N,N′, disuccinimidyl carbonate (or N-hydroxysuccinimide and dicylohexylcarbodiimide) and then reacting with compound 20.5.

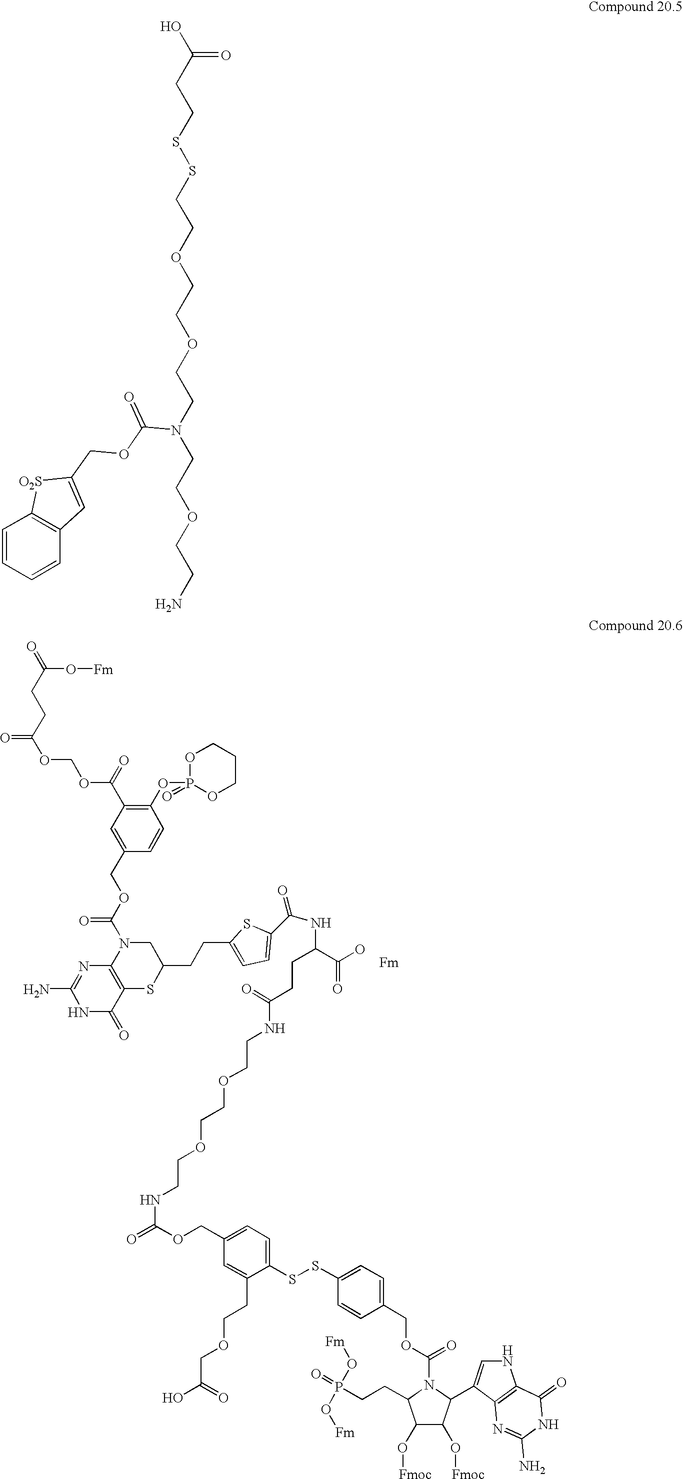

Compound 20.5 may be prepared by a multi-step procedure. [2-(2-Amino-ethoxy)-ethyl]-trityl-amine may be reacted with 2-[2-(2-Chloro-ethoxy)-ethoxy]-ethanol in an inert solvent in the presence of base and 2-[2-(2-{2-[2-(Trityl-amino)-ethoxy]-ethylamino}-ethoxy)-ethoxy]-ethanol isolated by chromatography.

Treatment with di-t-butyl pyrocarbonate and in an inert solvent will give {2-[2-(2-Hydroxy-ethoxy)-ethoxy]-ethyl}-{2-[2-(trityl-amino)-ethoxy]-ethyl}-carbamic acid tert-butyl ester. Treatment with tosyl chloride and base will give toluene-4-sulfonic acid 2-{2-[2-(tert-butoxycarbonyl-{2-[2-(trityl-amino)-ethoxy]-ethyl}-amino)-ethoxy]-ethoxy}-ethyl ester. Treatment with sodium hydrogen sulfide in an inert solvent will give {2-[2-(2-Mercapto-ethoxy)-ethoxy]-ethyl}-{2-[2-(trityl-amino)-ethoxy]-ethyl}-carbamic acid tert-butyl ester. Then the mixed disulfide with 3-Mercapto-propionic acid 9H-fluoren-9-ylmethyl ester may then be formed by a variety of previously referenced methods. The product 3-(2-{2-[2-(tert-butoxycarbonyl-{2-[2-(trityl-amino)-ethoxy]-ethyl}-amino)-ethoxy]-ethoxy}-ethyldiulfanyl)-propionic acid 9H-fluoren-9-ylmethyl ester may then be treated with acid to selectively remove the trityl group and then reacted with 9-fluorenylmethyl chloroformate and base to give 3-(2-{2-[2-(tert-Butoxycarbonyl-{2-[2-(9H-fluoren-9-ylmethoxycarbonylamino)-ethoxy]-ethyl}-amino)-ethoxy]-ethoxy}-ethyldisulfanyl)-propionic acid 9H-fluoren-9-ylmethyl ester. Treatment with trifluoracetic acid will remove the t-Boc group. Treatment with 1,1-dioxobenzo[b]thiophene-2-yl-methoxycarbonyl chloride and a base such as pyridine will give 3-(2-{2-[2-((1,1-dioxo-1H-1λ6-benzo[b]thiophen-2-ylmethoxycarbonyl)-{2-[2-(9H-fluoren-9-yl-methoxycarbonylamino)-ethoxy]-ethyl}-amino)-ethoxy]-ethoxy}-ethyldisulfanyl)-propionic acid 9H-fluoren-9-ylmethyl ester. Selective removal of the Fmoc group and Fm ester with a hindered base will give compound 20.5.

Compound 20.6 may be prepared by coupling compound 20.7 and 20.8.

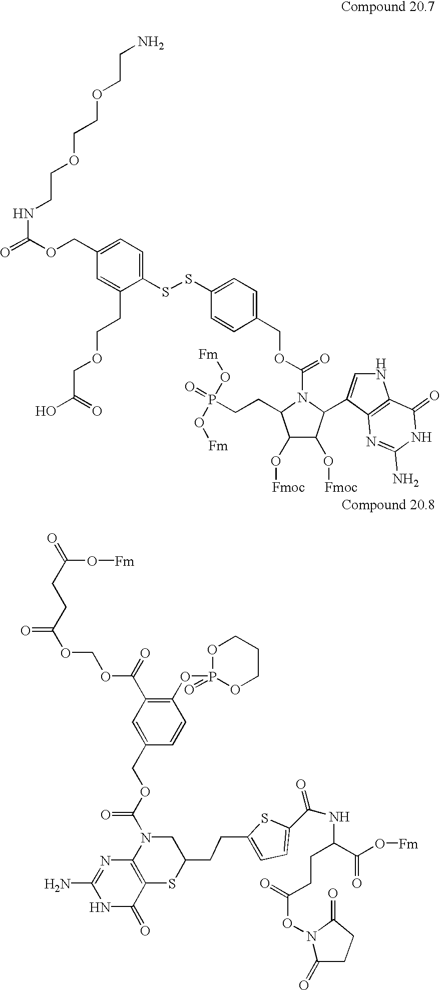

Compound 20.7 may be prepared by a multi-step procedure. Reacting {2-[2-(2-Amino-ethoxy)-ethoxy]-ethyl}-trityl amine and compound 20.7a, followed by treatment with acetic acid to remove the trityl group, and followed by treatment with tris(2-aminoethyl)amine to selectively cleave the Bsm ester, will give compound 20.7.

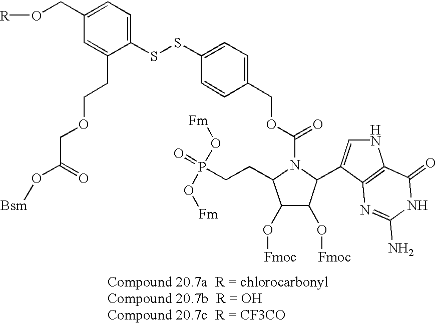

Compound 20.7a may be prepared by treating compound 20.7b with phosgene in an inert solvent. Compound 20.7b may be prepared by the selective removal of the triflouroacetate group from compound 20.7c with aqueous base. Compound 20.7c may be prepared by reacting compound 20.7d and compound 20.9 in an inert solvent in the presence of a base such as pyridine.

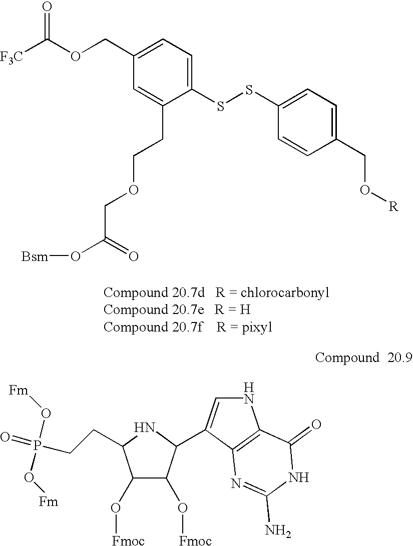

Compound 20.7d may be prepared by treating compound 20.7e with phosgene in an inert solvent. Compound 20.7e may be prepared by treating compound 20.7f with dilute trifluororacetic acid in an inert solvent to remove the pixyl group. Compound 20.7f may be prepared by reacting compound 20.7g with trifloroacetic anhydride and base and then treating the product with 1,1-dioxobenzo[b]thiophene-2-yl-methanol and a reagent such as dicyclohexylcarbodiimide in an inert solvent.

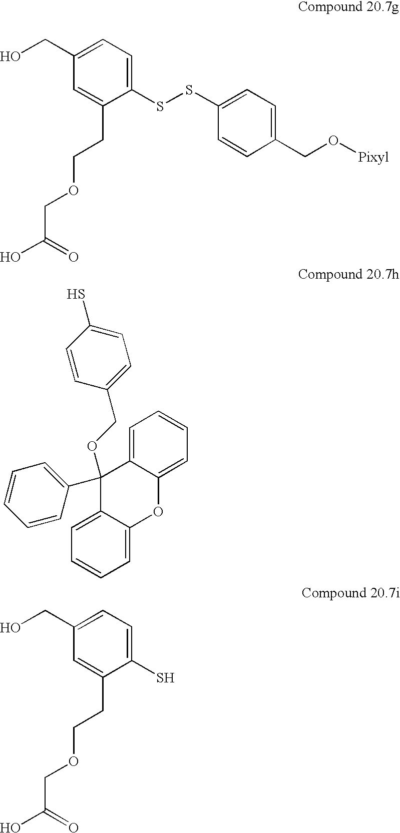

Compound 20.7g may be prepared by reacting diethyl azidocarboxylate with compound 20.7h and then reacting the product with compound 20.7i to form the mixed disulfide. The following references relate to this subject matter: Mukaiyama T.; Takahashi K., “A Convenient Method for the Preparation of Unsymmetrical Disulfides by the use of Diethyl Azodicarboxylate,” Tetrahedron Letters, 56:5907-5908 (1968), the contents of which are incorporated herein by reference in their entirety.

Compound 20.7h may be prepared by reacting 2 equivalents of 9-chloro-9-phenyl-9H-xanthene (pixyl chloride) with 4-mercapto phenol disulfide and base and then reducing the disulfide with an agent such as sodium borohydride.

Compound 20.7i may be prepared by a multi-step procedure. A Friedel-Crafts reaction between 4-mercapto-benzoic acid and chlorocarbonylmethoxy-acetic acid methyl ester will give 4-mercapto-3-(2-methoxycarbonylmethoxy-acetyl)-benzoic acid. Reduction of the ketone with Zn/HCL will give 4-mercapto-3-(2-methoxycarbonylmethoxy-ethyl)-benzoic acid. Treatment with borane in a solvent such as tetrahydrofuran will reduce the carboxylic acid to the alcohol. Hydrolysis of the methyl ester will give compound 20.7i. The following references relate to this subject matter: Gore P. H., “Aromatic Ketone Synthesis, in” Friedel-Crafts and Related Reactions, Olah G. A. (edt.), John Wiley & Sons, p.55 (1964); Read R. R.; Wood J. Jr., “o-n-Heptylphenol,” Org Syn Coll Volume 3, pp. 444-446; Yoon N. M.; Pak C. S., “Selective Reductions. XIX. The Rapid Reaction of Carboxylic Acids with Borane-Tetrahydrofuran. A Remarkable Convenient Procedure for the Selective Conversion of Carboxylic Acids to the Corresponding Alcohols in the Presence of Other Functional Groups,” J Org Chem, 33(16):2786-2792 (1973), the contents of which are incorporated herein by reference in their entirety.

Compound 20.9 may be prepared by a multi-step method. Compound 20.9.1 is a known compound. The following references relate to this subject matter: U.S. Pat. No. 6,066,722 May 23, 2000 Furneaux et al., “Inhibitors of Nucleoside Metabolism”. Shi W., et al., “The 2.0 A Structure of Human Hypoxanthine-guanine Phosphoribosyltransferase in Complex with a Transition-state Analog Inhibitor,” Nature Structural Biology, 6(6):588-593 (1999), the contents of which are incorporated herein by reference in their entirety.

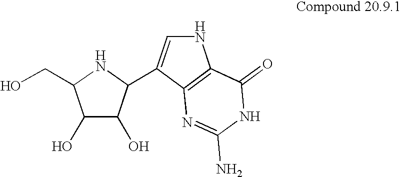

Treatment of compound 20.9.1 with trityl chloride to protect the 5′ hydroxy group, followed by treatment with benzyloxycarbonyl chloride (Cbz chloride) in an inert solvent, in the presence of a base, such as pyridine base will give compound

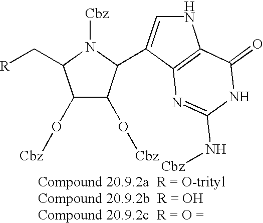

Treatment with acid will remove the trityl group and give compound 20.9.2b. Oxidation with dimethylsulfoxide and an agent such as dicyclohexylcarbodiimide will give the aldehyde (compound 20.9.2c). Reaction with [diisopropyll)-methylidene] triphenylphosphorane followed by catalytic hydrogenation with palladium on carbon will give compound 20.10a. The following references relate to this subject matter: Xu Y., et al., “Preparation of New Wittig Reagents and Their Application to the Synthesis of αβ-Unsaturated Phosphonates,” J Org Chem, 61:7697-7701 (1996); Montgomery J. A.; Thomas H. J., “Phosphonate Analogue of 2′-Deoxy-5-fluorouridylic Acid,” J Med Chem, 22(1):109-111 (1979), the contents of which are incorporated herein by reference in their entirety.

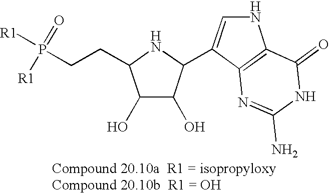

Treatment of compound 20.10a with hydrochloric acid will give compound 20.10b as the hydrochloride salt. Treatment with 2-(4-methoxybenzyloxycarbonyloxyimino)-2-phenyl acetonitrile and base will give compound 20.10c.

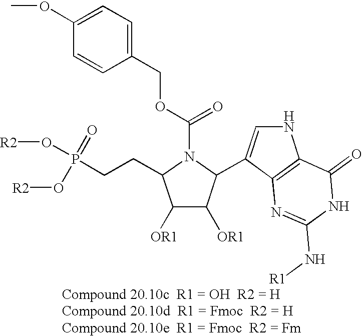

Treatment of compound 20.10c with 9-fluorenylmethyl chloroformate and base in an inert solvent will give compound 20.10.d. Compound 20.10d may be converted into the bis 9-fluorenylmethyl ester by treatment with (9H-Fluoren-9-yl)-methanol and a condensing reagent such as 1-mesitylenesulphonyl chloride. Alternatively, compound 20.10d may be converted into the dichlorophosphonate derivative by reagents such as oxalyl chloride/dimethylformamide and reacted with (9H-Fluoren-9-yl)-methanol and base to give compound 20.10e. Treatment of compound 20.10e with acid will remove the p-methoxybenzylcarbonyl protecting group and will give compound 20.9.

Compound 20.8 may be prepared by a multi-step process. The following references relate to this subject matter: Varney M. D., et al., “Protein Structure-Based Design, Synthesis, and Biological Evaluation of 5-Thia-2,6-diamino-4(3H)-oxopyrimidines: Potent Inhibitors of Glycinamide Ribonucleotide Transformylase with Potent Cell Growth Inhibition,” J Med Chem, 40:2502-2524 (1997), the contents of which are incorporated herein by reference in their entirety.

Treatment of compound 20.8.1 with compound 20.8.2 in the presence of base in an inert solvent will give compound 20.8.3.

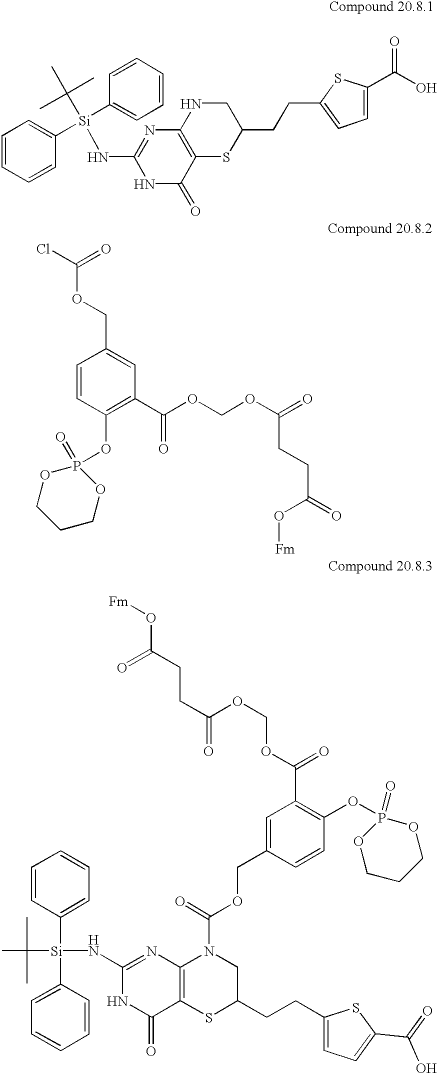

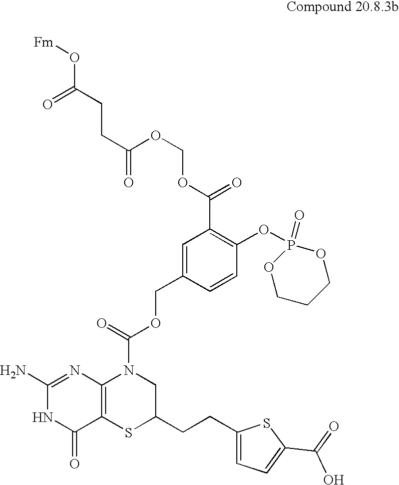

The silyl based protecting group may be removed with a reagent such as pyridine-HF to give compound 20.8.3b. Compound 20.8.3b may be coupled with compound 20.8.3c and the trichloroethyl ester cleaved with Zn and acid to give compound 20.8.3d.

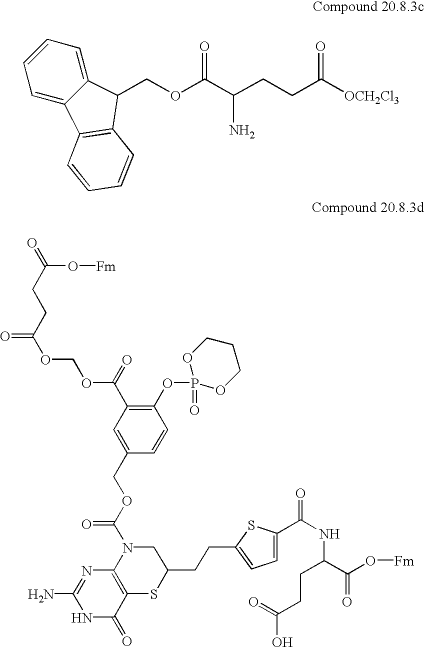

Treating compound 20.8.3d with dicyclohexylcarbodiimide and N-hydryoxy-succinimide in an inert solvent will give compound 20.8.

Compound 20.8.3c may be prepared by a multi-step process. The known compound L-N-t-Boc glutamic acid a (9H-Fluoren-9-yl)-methyl ester may be coupled with 2,2,2,trichloroethanol with a reagent such as dicyclohexylcarbodiimide in an inert solvent. Treatment with acid will remove the t-Boc group and give compound 20.8.3c as the salt.

Compound 20.2b may be prepared by a multi-step process. Coupling compound 20.2.11a and compound 17.4b, followed by removal of the trichloroethylcarbonyl protecting group with Zn and acid, followed by coupling compound 20.2.2a, followed by selective removal of the Bsmoc group with tris(2-aminoethyl)-amine will give compound 20.2b.

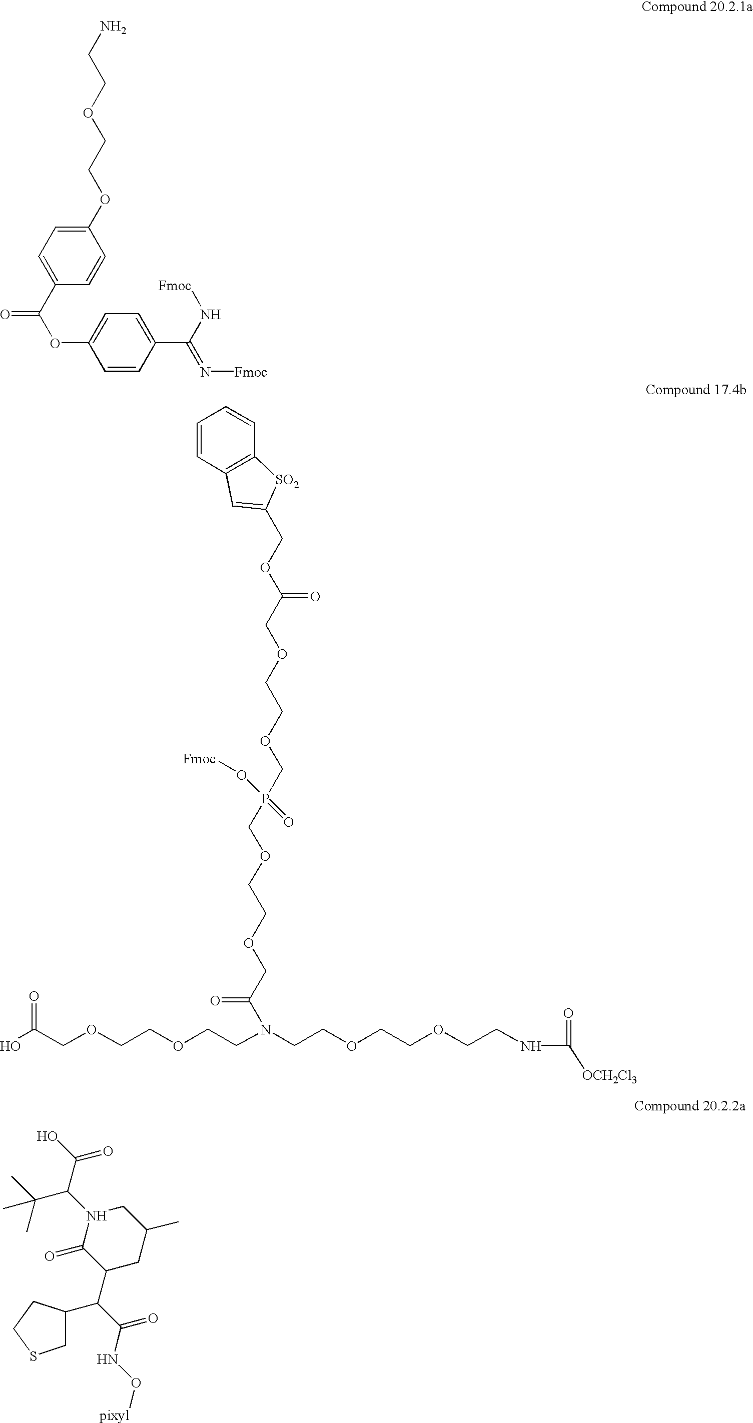

Compound 20.2.1a may be prepared by coupling compound 20.2.1b and 20.2.1c and then reducing the azido group to an amino group. The reduction of the azido group may be carried out by a variety of methods including catalytic hydrogenation with palladium on carbon, or triphenyl phosphine water.

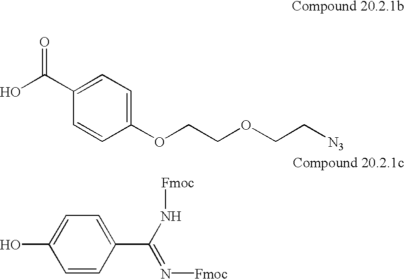

Compound 20.2.1b may be prepared by a multi-step process. Reacting 2-(2-Chloro-ethoxy)-ethanol and 4-Hydroxy-benzoic acid tert-butyl ester in the presence of base in an inert solvent will give 4-[2-(2-Hydroxy-ethoxy)-ethoxy]-benzoic acid tert-butyl ester. Treatment with tosyl chloride and base followed by the reaction with lithium azide will give 4-[2-(2-azido-ethoxy)-ethoxy]-benzoic acid tert-butyl ester. The tert-butyl ester may then be cleaved with acid to give compound 20.2.1b.

Compound 20.2.1c may be prepared by treating 4-hydroxy-benzamidine with a silylating agent such as chlorotrimethylsilane and base or hexamethyidisilazane and then treating with 9-fluorenylmethyl chloroformate and base, and hydrolyzing the trimethylsilyl ether group.

Compound 20.2.2a may be prepared by a multi-step procedure. The known compound 20.2.2b may be converted into the methyl ester compound 20.2.2c by routine methods. The following references relate to this subject matter: Praft L. M., et al., “The Synthesis of Novel Matrix Metalloproteinase Inhibitors Employing the Ireland-Claisen Rearrangement,” Bioorg Med Chem Lett, 8:1359-1364 (1998), the contents of which are incorporated herein by reference in their entirety.

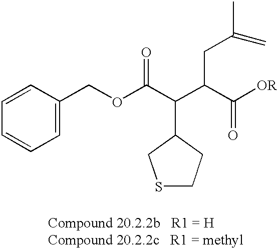

Catalytic hydrogenation with palladium on carbon will give compound 20.2.2d. Using routine methods the t-butyl ester compound 20.2.2e may be prepared. Selective hydrolysis of the methyl ester with aqueous sodium hydroxide will give compound 20.2.2f.

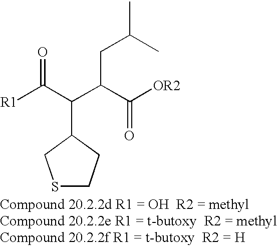

Coupling with compound 20.2.2f and compound 20.2.2g will give compound 20.2.2h.

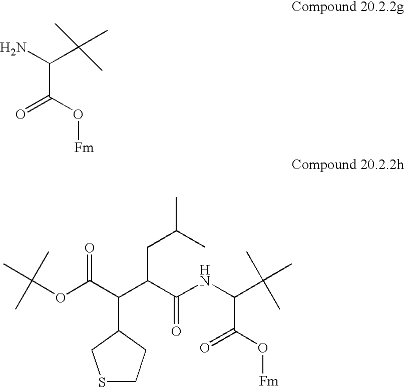

Treatment with acid will cleave the t-butyl ester and give compound 20.2.2i. Coupling with O-trimethylsilyl hydroxylamine and an agent such as dicyclohexylcarbodiimide will give, after hydrolysis, compound 20.2.2j.

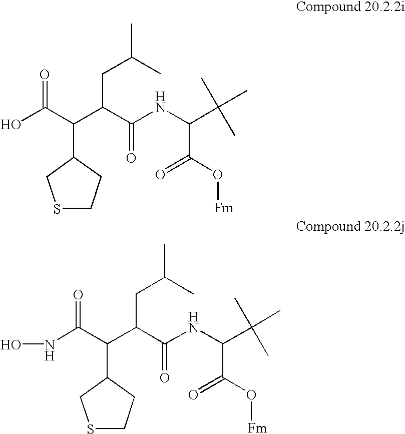

Treatment with 9-chloro-9-phenyl-9H-xanthene and base will give compound 20.2.2a.

### Example 21

Compound 21 is a multifunctional drug delivery vehicle that is targeted against tumor cells that express urokinase, matrix metalloproteinases (1, 2, 3, 9, and MT-MMP-1) and gastrin releasing peptide receptor. Individually each ligand will bind at nanomolar concentrations to its receptor. Accordingly, binding of any two of the ligands should give essentially irreversible binding to the tumor cell. The drug has a masked folic acid group as an intracellular transport ligand. The drug will release Phthalascidin a cytotoxin that has an IC₅₀ in the 0.1-1 nM range. The phathaloscidin is linked to the drug complex by a carbamate group that will undergo cleavage upon reduction of a disulfide bond. The following references relate to this subject matter: Martinez EJ, et al., “Phthalascidin, A Synthetic Antitumor Agent with Potency and Mode of Action Comparable to Ecteinascidin 743,” Proc Natl Acad Sci USA, 96:3496-3501 (1999); Ashwood V., et al., “PD 176252—The First High Affinity Non-Peptide Gastrin-Releasing Peptide (BB2) Receptor Antagonist,” Bioorg Med Chem Lett, 8(18):2589-94 (1998); WO 98/07718 Feb. 26, 1998Horwell et al., “Non-Peptide Bombesin Receptor Antagonist”, the contents of which are incorporated herein by reference in their entirety.

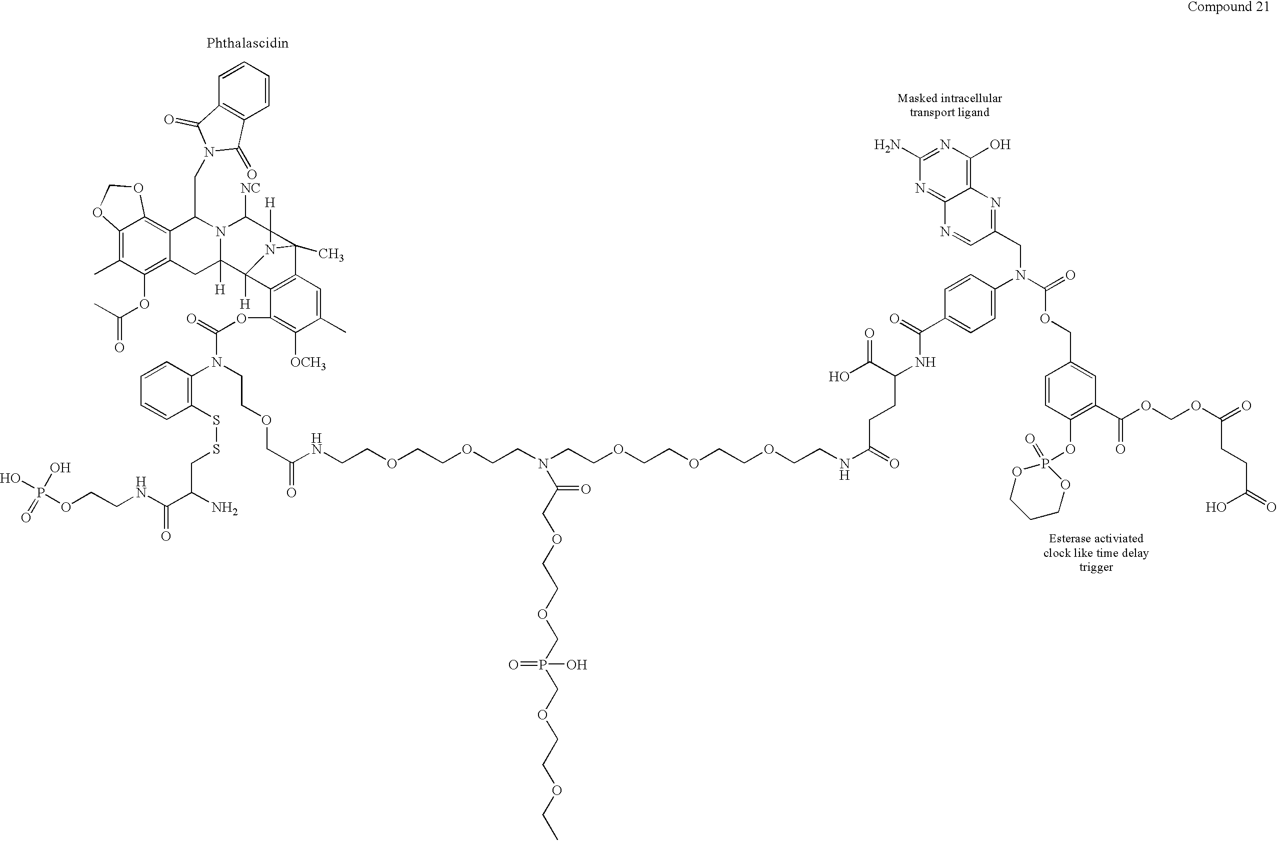

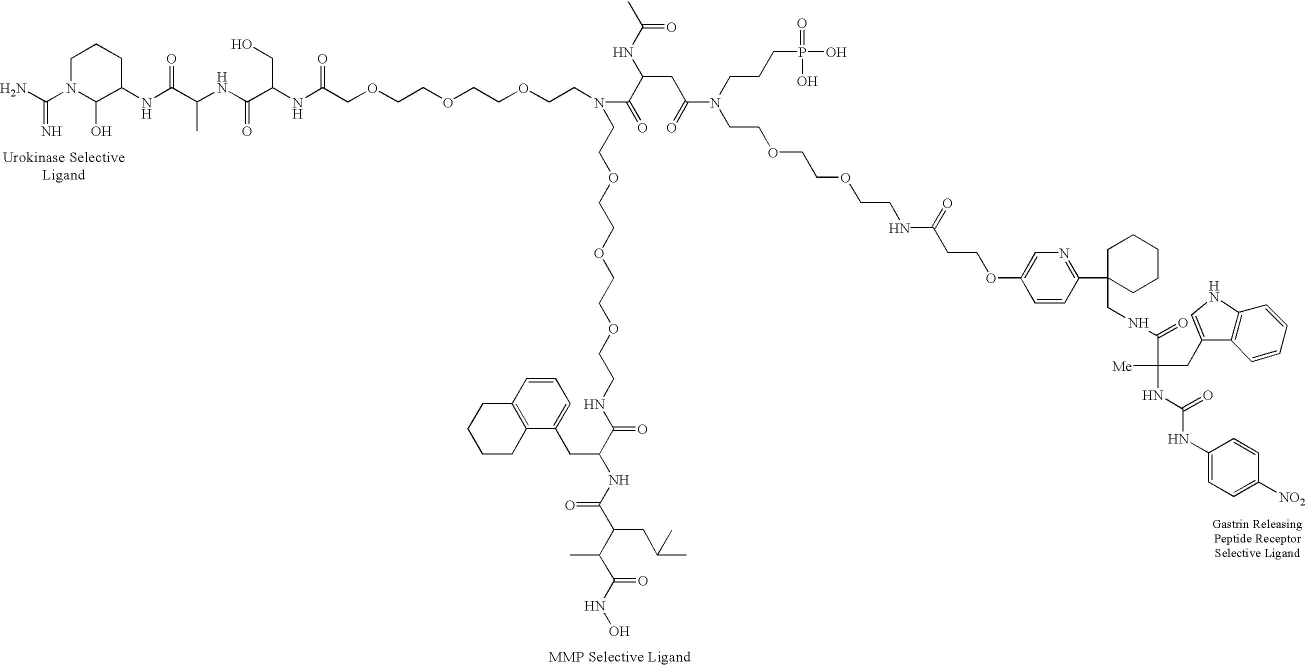

Compound 21 may be prepared by deprotecting compound 21a with tetrabutylammonium fluoride to remove both the silyl and fluorenyl based protecting groups. A deblocking step with dilute acid is also required. Alternatively, a variety of other reagents known to cleave t-butyidimethylsilyl ethers may be employed. The Fmoc and fluorenylmethyl esters may be cleaved with base.

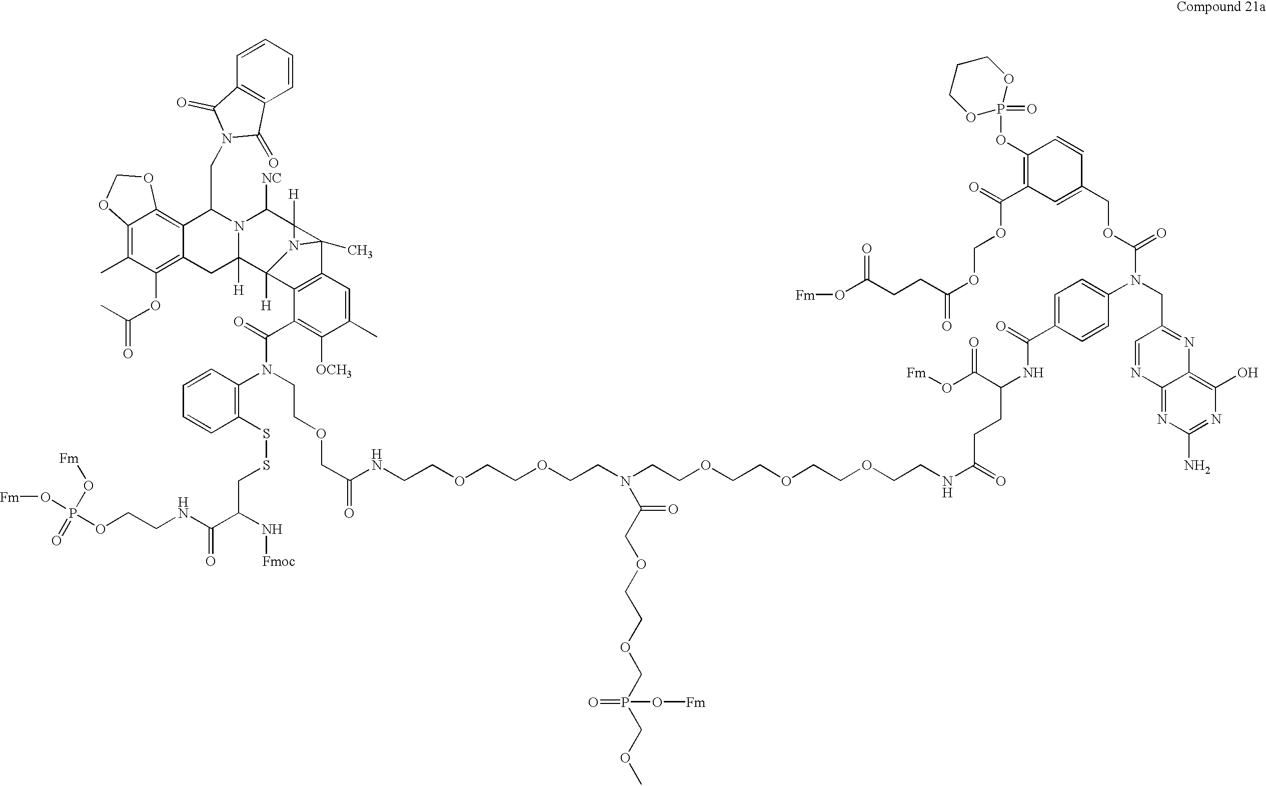

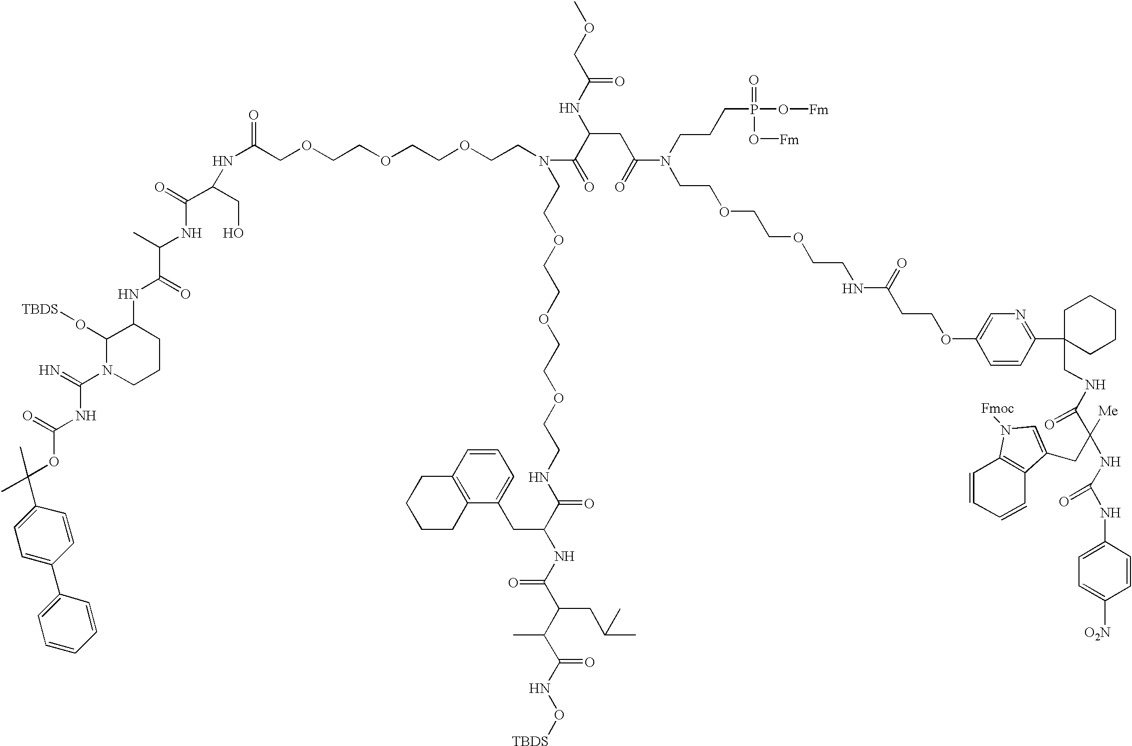

Compound 21a may be prepared by coupling compound 21.1 and compound 21.2.

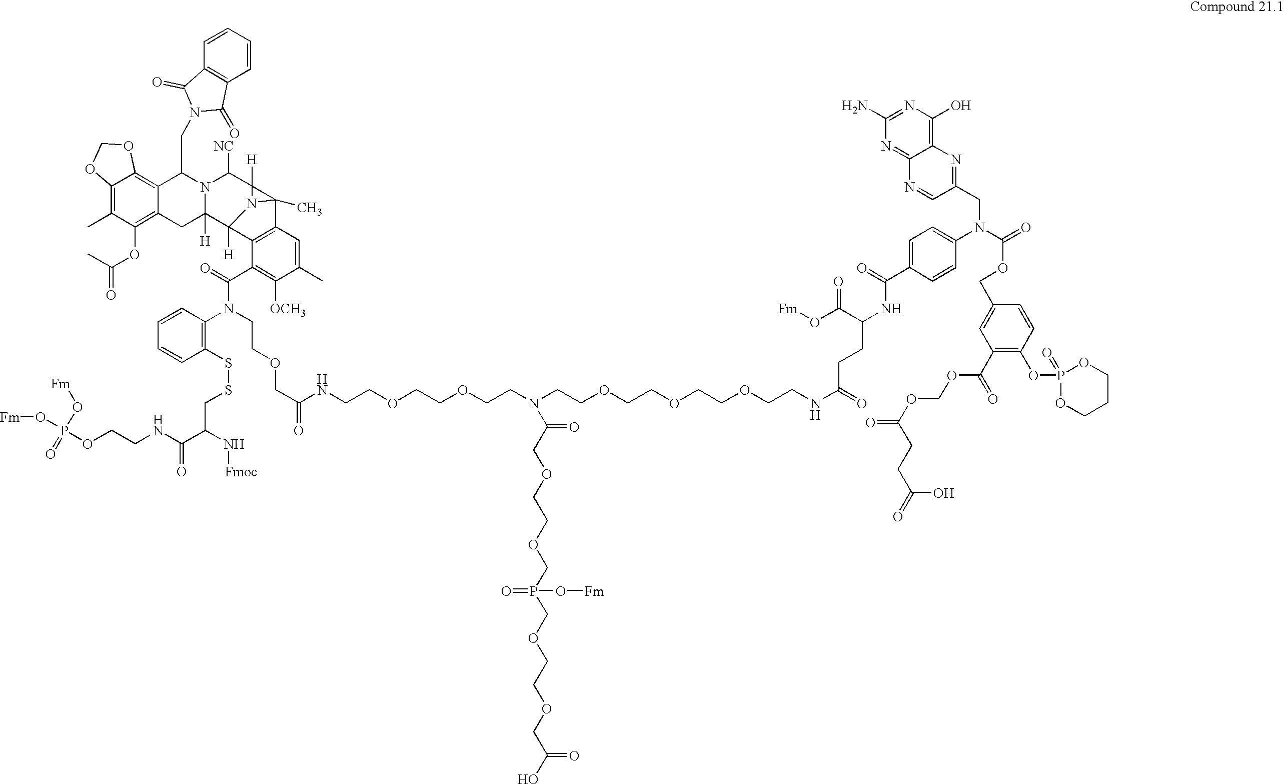

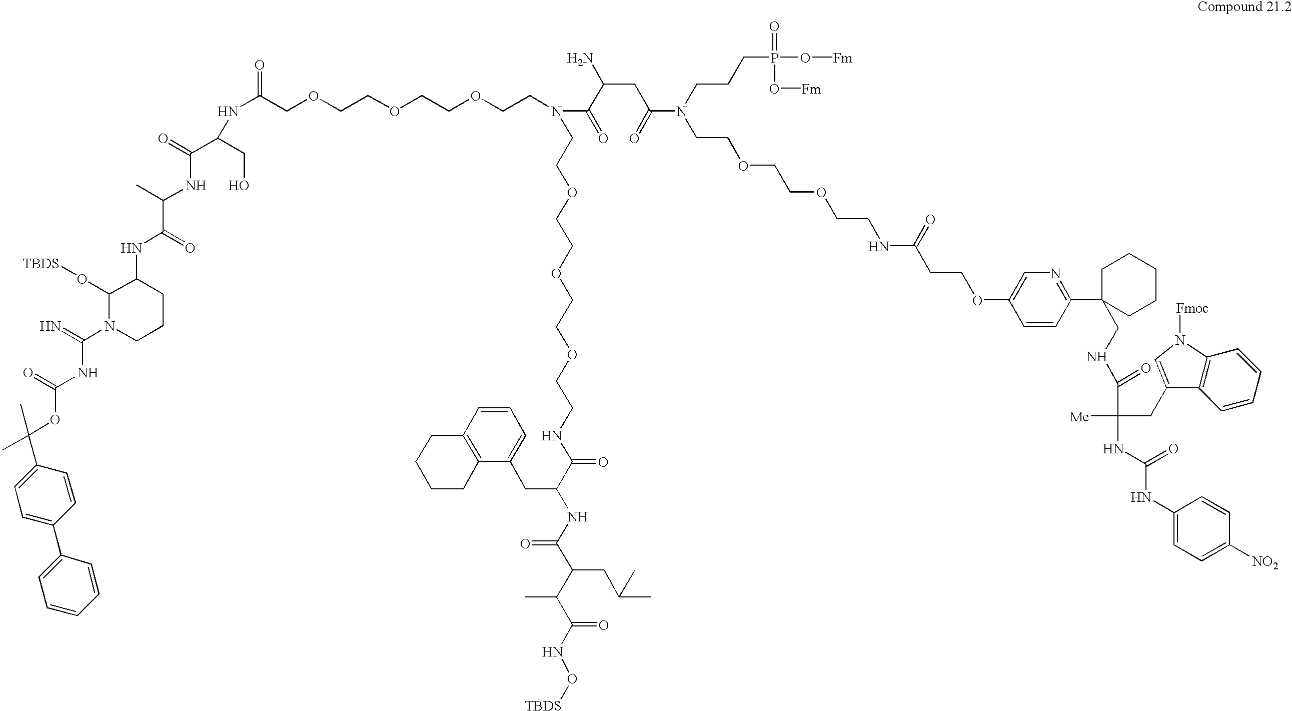

Compound 21.1 may be prepared by a multi-step process. Coupling compounds 21.1.1 and 21.1.2, followed by removal of the p-methoxy-benzyloxycarbonyl protecting group with dilute trifluoroacetic acid in an inert solvent, followed by coupling with compound 6.2.0b, followed by selective cleavage of the Bsm group with tris(2-aminoethyl)-amine will give compound 21.1.

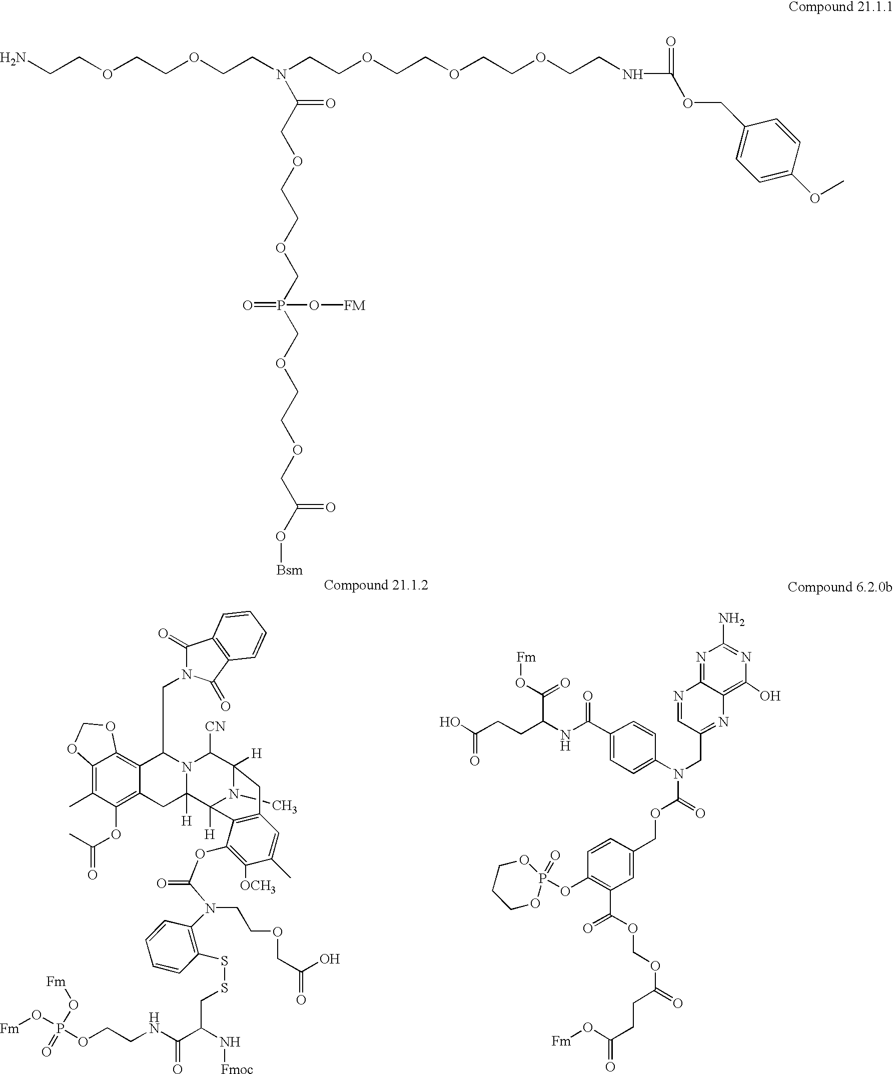

Compound 21.1.1 may be prepared by coupling compounds 21.1.1a and 21.1.1b and then removing the trichloroethoxycarbonyl group with Zn and phosphate buffer or Zn and dilute acid.

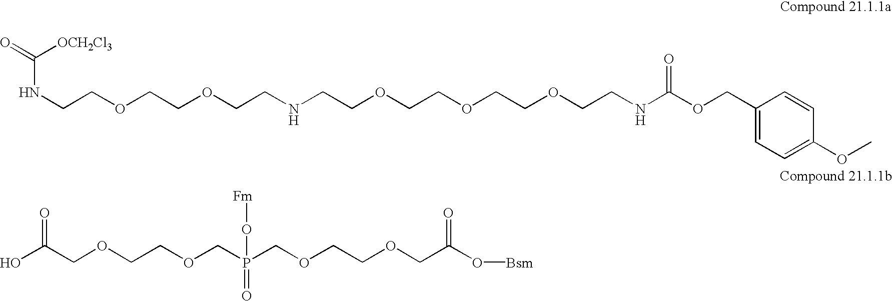

The synthesis of compound 21.1.1b was described previously. Compound 21.1.1a may be prepared by a multistep procedure. 2-[2-(2-Amino-ethoxy)-ethoxy]-ethanol may be treated with 2,2,2,trichloroethylchloroformate and base to give {2-[2-(2-Hydroxy-ethoxy)-ethoxy]-ethyl}-carbamic acid 2,2,2-trichloro-ethyl ester. Treatment with tosyl chloride and base will give toluene-4-sulfonic acid 2-{2-[2-(2,2,2-trichloro-ethoxycarbonylamino)-ethoxy]-ethoxy}-ethyl ester. Reaction with (2-{2-[2-(2-Amino-ethoxy)-ethoxy]-ethoxy}-ethyl)-carbamic acid 4-methoxybenzyl ester and base in an inert solvent will give, after purification, compound 21.1.1a.

Compound 21.1.2 may be prepared by reacting compounds 21.1.2a and Phthalascidin in an inert solvent in the presence of a base such as pyridine.

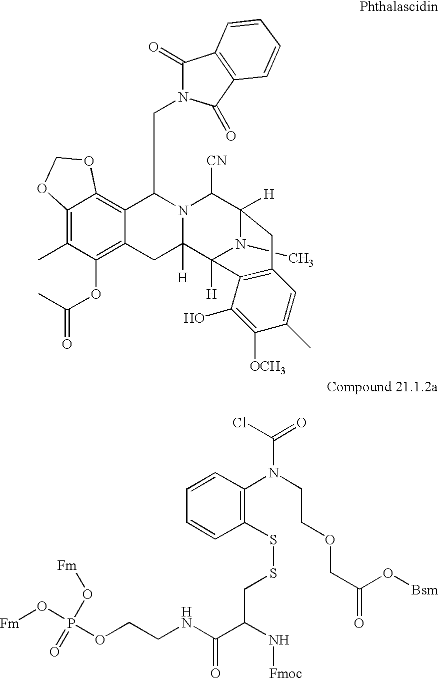

Compound 21.1.2a may be prepared by treating compound 21.1.2b with phosgene in an inert solvent.

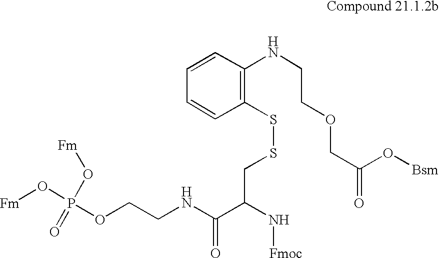

Compound 21.1.2b may be prepared by a multistep process. Reacting diethyl azidocarboxylate with compound 14.11.4 and then reacting the product with compound 17.11.1a will form the mixed disulfide.

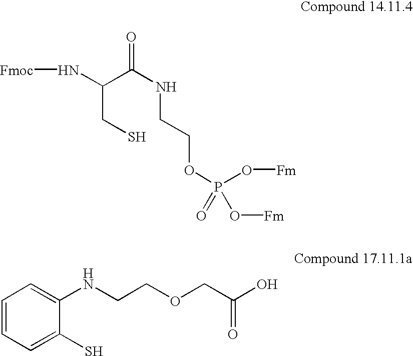

The product may then be treated with di-t-butyl pyrocarbonate in an inert solvent. Treating with (1,1-Dioxo-1H-1λ6-benzo[b]thiophen-2-yl)-methanol and dicyclohexylcarbodiimide in an inert solvent, followed by acid treatment to remove the t-Boc group will give compound 21.1.2b.

Compound 21.2 may be prepared by coupling compounds 21.2.1a and 21.2.2a and then treating with tris(2-aminoethyl)-amine to selectively cleave the Bsmoc group.

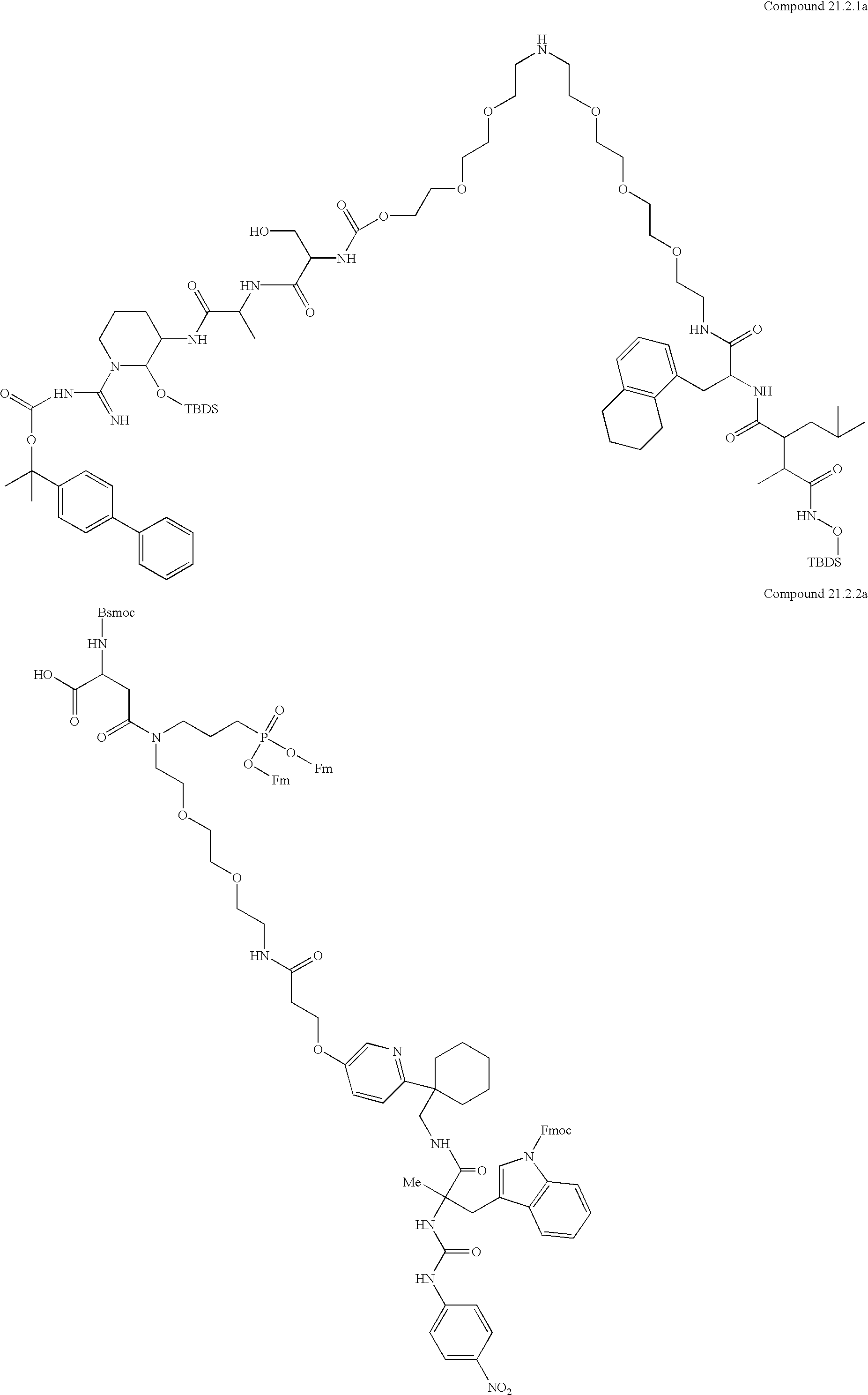

Compound 21.2.1a may be prepared by a multi-step process. Compound 21.2.1b and 21.2.1c may be coupled and then the product may be treated with Zn phosphate buffer to remove the trichloroethoxycarbonyl protecting group. Coupling with compound 21.2.1d followed by the removal of the Bsmoc group with tris(2-aminoethyl)-amine will give compound 21.2.1a.

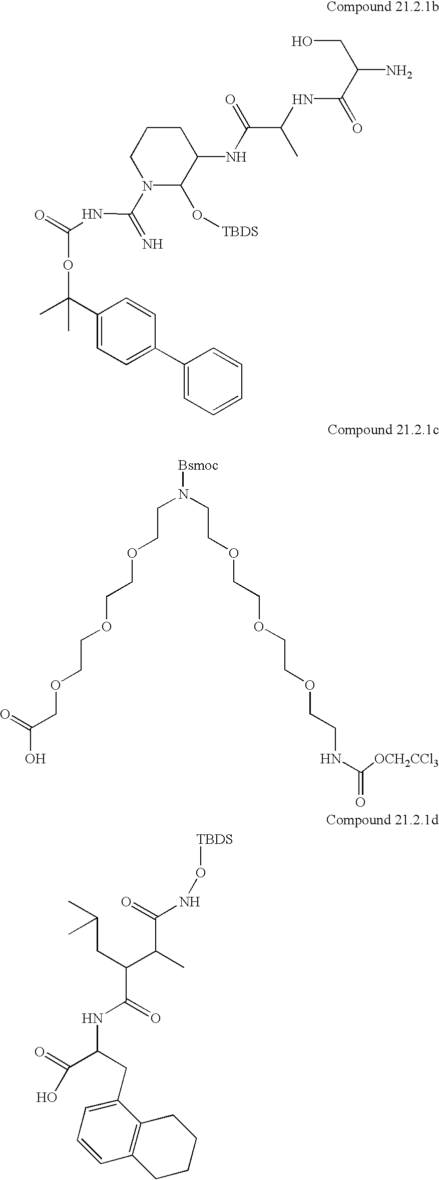

The synthesis of compound 21.2.1b was given previously as compound 14.7.

Compound 21.2.1c may be prepared by a multi-step process. 2-{2-[2-(2-Amino-ethoxy)-ethoxy]-ethoxy}-ethylamine may be treated with trityl chloride and base and (2-{2-[2-(2-Amino-ethoxy)-ethoxy]-ethoxy}-ethyl)-trityl-amine isolated. Alkylation with {2-[2-(2-Chloro-ethoxy)-ethoxy]-ethoxy}-acetic acid in an inert solvent in the presence of base will give [2-(2-{2-[2-(2-{2-[2-(trityl-amino)-ethoxy]-ethoxy}-ethoxy)-ethylamino]-ethoxy}-ethoxy)-ethoxy]-acetic acid. Treating with (1,1-Dioxo-1H-1λ6-benzo[b]thiophen-2-yl)-methyl chloroformate and base in an inert solvent will give compound 21.2.1c.2.

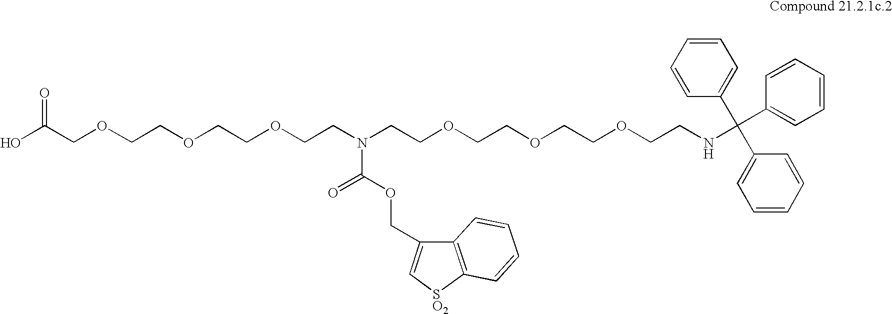

Treating with acid to remove the trityl group followed by the reaction with 2,2,2 trichloroethyl chloroformate and base in an inert solvent or under Schoften-Bauman conditions will give compound 21.2.1c.

Compound 21.2.1d may be prepared by a multi-step procedure from compound 14.6.5. Coupling of compound 14.6.5 with 2,2,2 trichloroethanol will give compound 21.2.1d.1. Treatment with acid will cleave the t-butyl ester and give compound 21.2.1d.2. Treatment with (9H-Fluoren-9-yl)-methanol and a coupling agent such as dicyclohexylcarbodiimide will give compound 21.2.2d.3. Treatment with Zn and acid will cleave the trichloroethyl ester and give compound 21.2.1d.4. Coupling with O-tert-butyl-dimethyl-silyl hydroxylamine will give compound 21.2.1d.5. Treatment with base will cleave the fluorenylmethyl ester and give compound 21.2.1d.

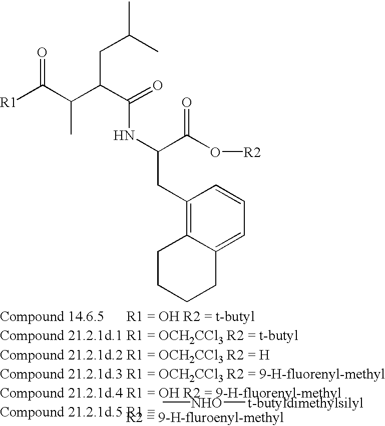

Compound 21.2.2a may be prepared by a multi-step process.

Compound 13b3 may be coupled with Bsmoc-L-aspartic acid α t-butyl ester in an inert solvent will give compound 21.2.2c.

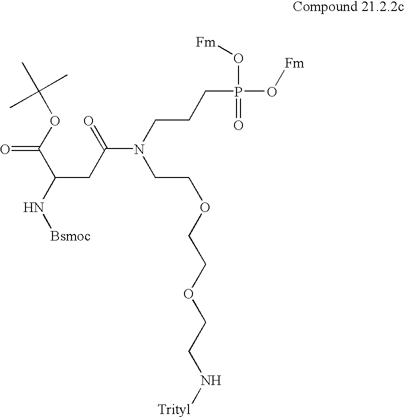

Treating with acid will remove the trityl group. The product may be coupled to compound 21.2.2e. Treatment with acid will cleave the t-Butyl ester group and give compound 21.2.2a.

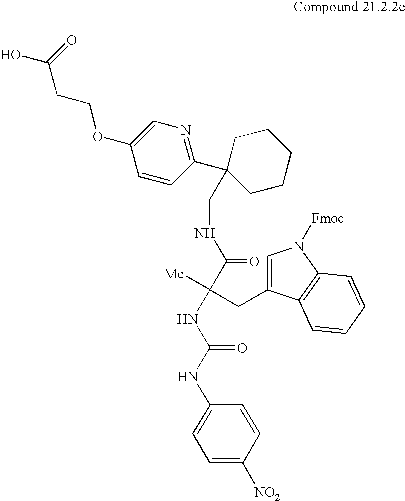

Compound 21.2.2e may be prepared by coupling compound 21.2.2f and compound 21.2.2g and treating with acid to cleave the t-butyl ester. The following reference relates to this subject matter: WO 98/07718, Feb. 26, 1998, Horwell et al., “Non-Peptide Bombesin Receptor Antagonist”, the contents of which are incorporated herein by reference in their entirety.

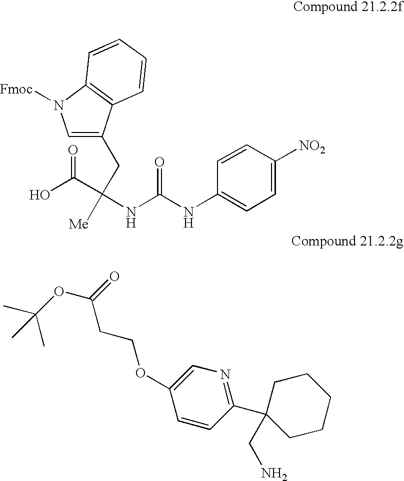

Compound 21.2.2g may be prepared by a multi-step process. 6-Methyl-pyridin-3-ol may be alkylated with 3-Bromo-propionic acid tert-butyl ester in an inert solvent in the presence of a strong base such as sodium hydride to give 3-(6-Methyl-pyridin-3-yloxy)-propionic acid tert-butyl ester. This can be oxidized with m-chloroperbenzoic acid to the corresponding N-oxide, which on reflux in acetic anhydride will rearrange to give 3-(6-Acetoxymethyl-pyridin-3-yloxy)-propionic acid tert-butyl ester. Treatment with sodium hydroxide will give 3-(6-Hydroxymethyl-pyridin-3-yloxy)-propionic acid tert-butyl ester. Treatment with tosyl chloride and base in an inert solvent followed by treatment with potassium cyanide will give 3-(6-cyanomethyl-pyridin-3-yloxy)-propionic acid tert-butyl ester. Alkylation with 1,5-dibromo-pentane, in the presence of a strong base such as sodium hydride, in an inert solvent will give 3-[6-(1-Cyano-cyclohexyl)-pyridin-3-yloxy]-propionic acid tert-butyl ester. Catalytic hydrogenation will give 3-[6-(1-aminomethyl-cyclohexyl)-pyridin-3-yloxy]-propionic acid tert-butyl ester (compound 21.2.2g). The following references relate to this subject matter: WO 98/07718 Feb. 26, 1998Horwell et al., “Non-Peptide Bombesin Receptor Antagonist”, the contents of which are incorporated herein by reference in their entirety.

### Example 22

Compound 22 is similar to compound 21, however a different gastrin releasing protein receptor selective ligand is employed. The following references relate to this subject matter: Karra S. R., et al., “^{99m}Tc-Labeling and in Vivo Studies of a Bombesin Analogue with a Novel Water-Soluble Dithiadiphosphine-Based Bifunctional Chelating Agent,” Bioconjugate Chem, 10(2):254-260 (1999), the contents of which are incorporated herein by reference in their entirety.

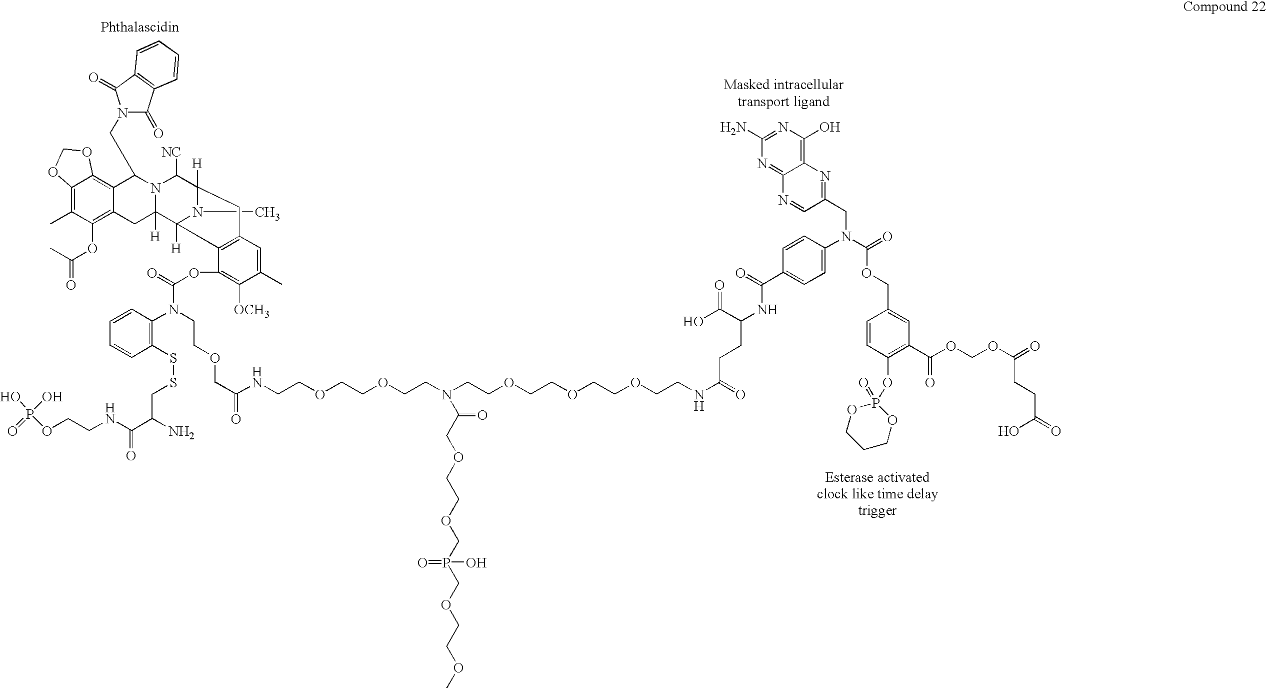

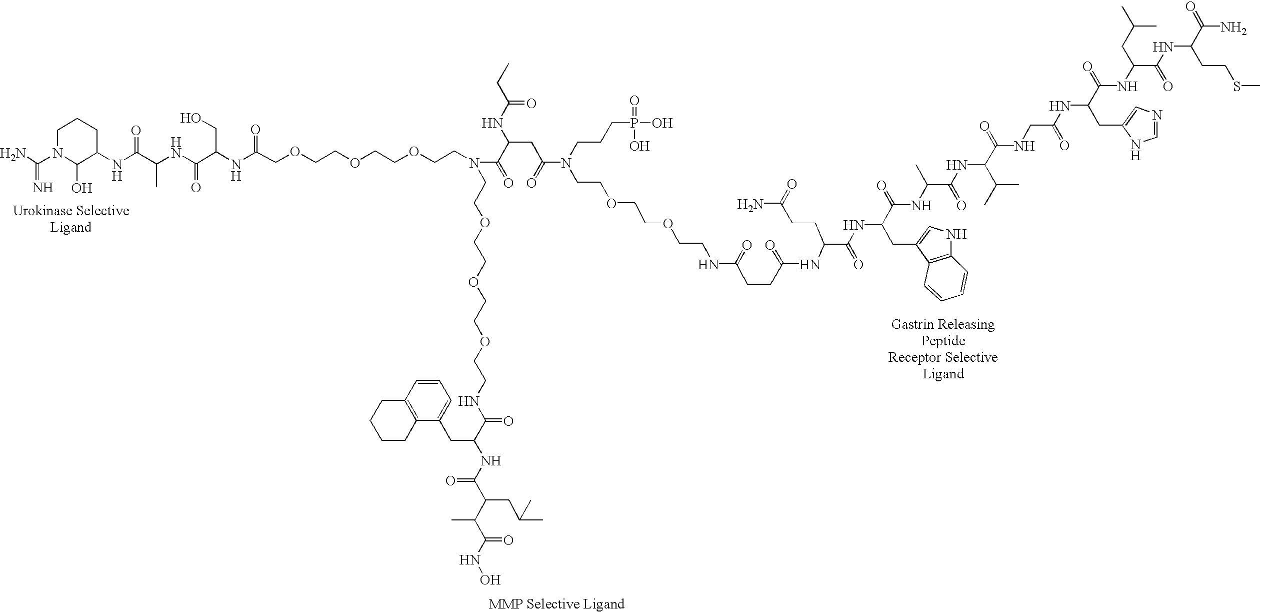

Compound 22 may be prepared by replacing compound 21.2.2e with compound 22.1 in the process described for the synthesis of compound 21.

Compound 22.1 may be prepared using routine methods of peptide synthesis.

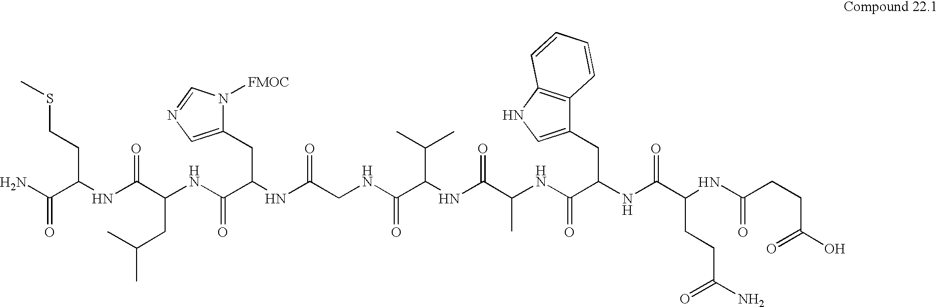

### Example 23

Compound 23 is a multifunctional drug delivery vehicle with targeting ligands for urokinase, matrix metalloproteinases (1, 2, 3, 9, and MT-MMP-1) and Gastrin/Cholecystokinin type B Receptors. The drug has a masked folic acid group as an intracellular transport ligand that will be activated by esterase. A derivative of cryptophycin that is toxic at picomolar concentrations will be freed upon cleavage of a disulfide trigger by thiol reductases. The following references relate to this subject matter: Showell G. A., et al., “High-Affinity and Potent, Water-Soluble 5-Amino-1,4-Benzodiazepine CCKB/Gastrin Receptor Antagonists Containing a Cationic Solubilizing Group,” J Med Chem, 37(6):719-21 (1994); Panda D., et al., “Antiproliferative Mechanism of Action of Cryptophycin-52: Kinetic Stabilization of Microtubule Dynamics by High-Affinity Binding to Microtubule Ends,” Proc Natl Acad Sci USA, 95:9313-9318 (1998); Smith C. D., et al., “Cryptophycin: A New Antimicrotubule Agent Active against Drug-resistant Cells,” Cancer Res, 54:3779-3784 (1994); Patel V. F., et al., “Novel Cryptophycin Antitumor Agents: Synthesis and Cytotoxicity of Fragment “B” Analogues,” J Med Chem, 42:2588-2603 (1999), the contents of which are incorporated herein by reference in their entirety.

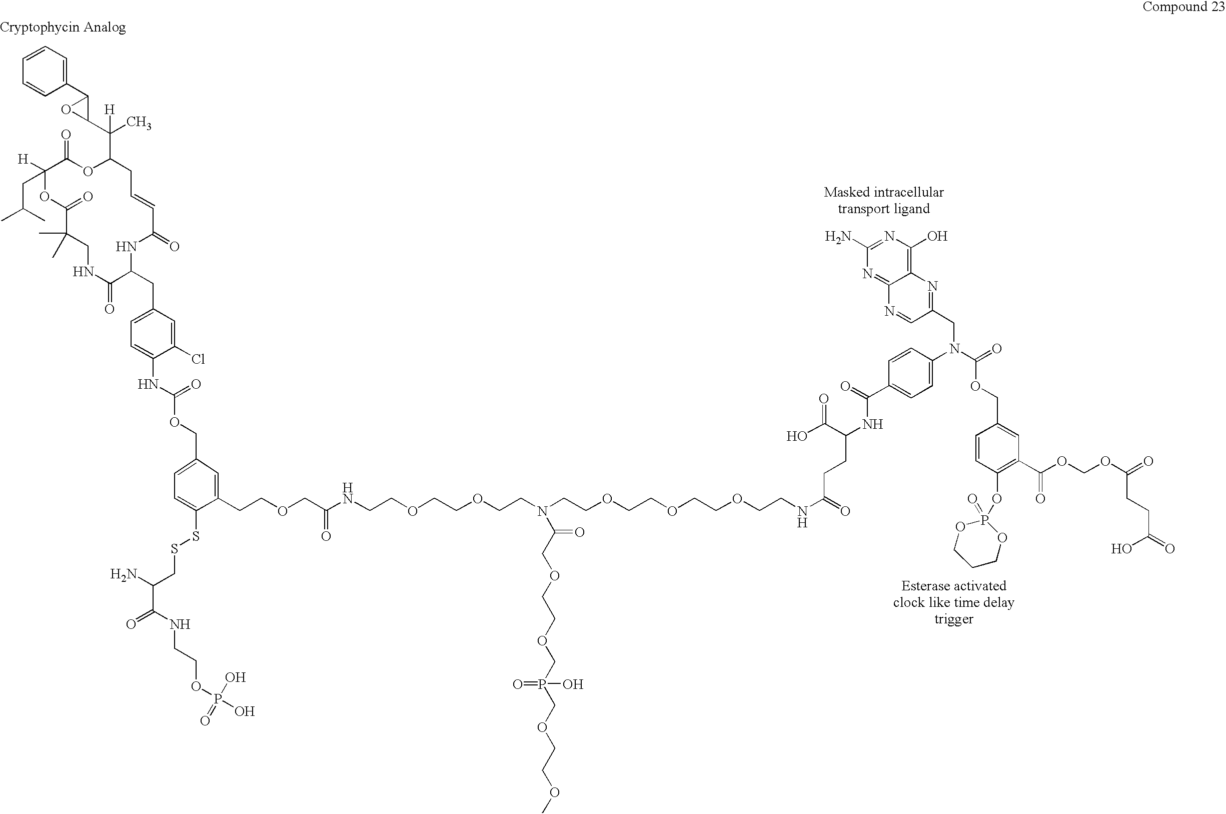

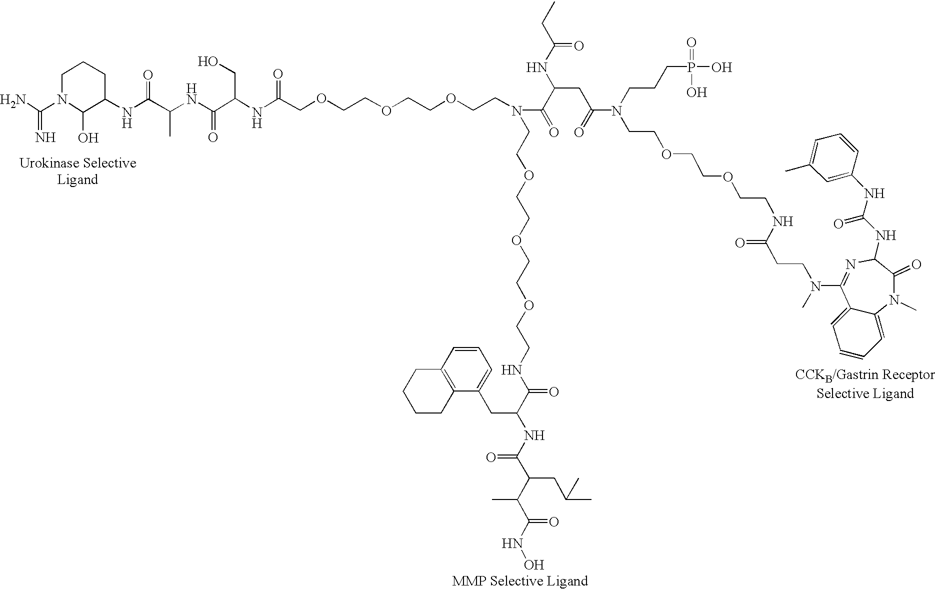

Compound 23 may be prepared by replacing compound 21.2.2e with compound 23.1 and replacing compound 21.1.2 with compound 23.2 as in the process described for the synthesis of compound 21.

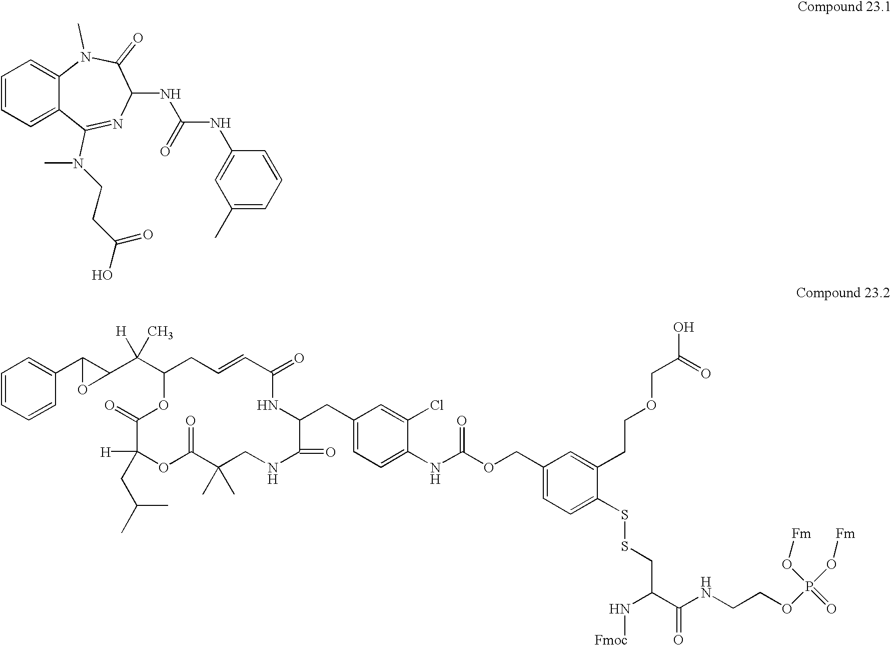

Compound 23.1 may be prepared by the methods decribed by Showell G. A. The following references relate to this subject matter: Showell G. A., et al., “High-Affinity and Potent, Water-Soluble 5-Amino-1,4-Benzodiazepine CCKB/Gastrin Receptor Antagonists Containing a Cationic Solubilizing Group,” J Med Chem, 37(6):719-21 (1994), the contents of which are incorporated herein by reference in their entirety.

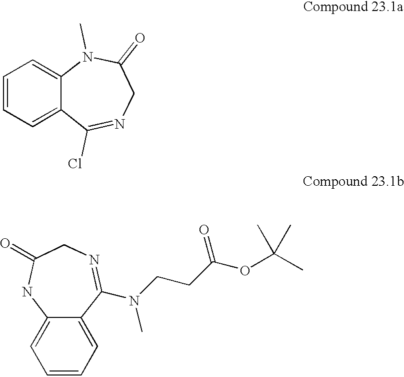

Treating compound 23.1a with 3-methylamino-propionic acid tert-butyl ester and a base such as triethylamine in an inert solvent will give compound 23.1b. Compound 21.3b may then be transformed into the t-butyl ester of compound 23.1 using the methods decribed by Showell G. A et al. Treatment with acid will cleave the t-butyl ester and give compound 23.1.

Compound 23.2 may be prepared by reacting 23.2a and 23.2b in an inert solvent in the presence of a base such as pyridine and then treating with tris(2-aminoethyl)-amine to selectively cleave the Bsm ester.

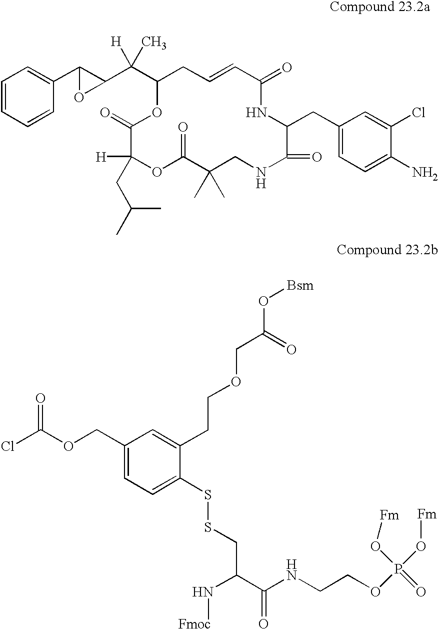

Compound 23.2a is a known compound. The following references relate to this subject matter: Patel V. F., et al., “Novel Cryptophycin Antitumor Agents: Synthesis and Cytotoxicity of Fragment “B” Analogues,” J Med Chem, 42:2588-2603 (1999), the contents of which are incorporated herein by reference in their entirety.

Compound 23.2b may be prepared by treating compound 14.11.3 with phosgene in an inert solvent.

### Example 24

Compound 24 is a multifunctional drug delivery vehicle with targeting ligands for urokinase, matrix metalloproteinases (1, 2, 3, 9, and MT-MMP-1) and melanocyte stimulating hormone receptor. The drug has a masked folic acid group as an intracellular transport ligand, which will be activated by esterase. A derivative of cryptophycin, which is toxic at picomolar concentrations, will be freed upon cleavage of a disulfide trigger by thiol reductases. The drug is expected to have activity against malignant melanoma.

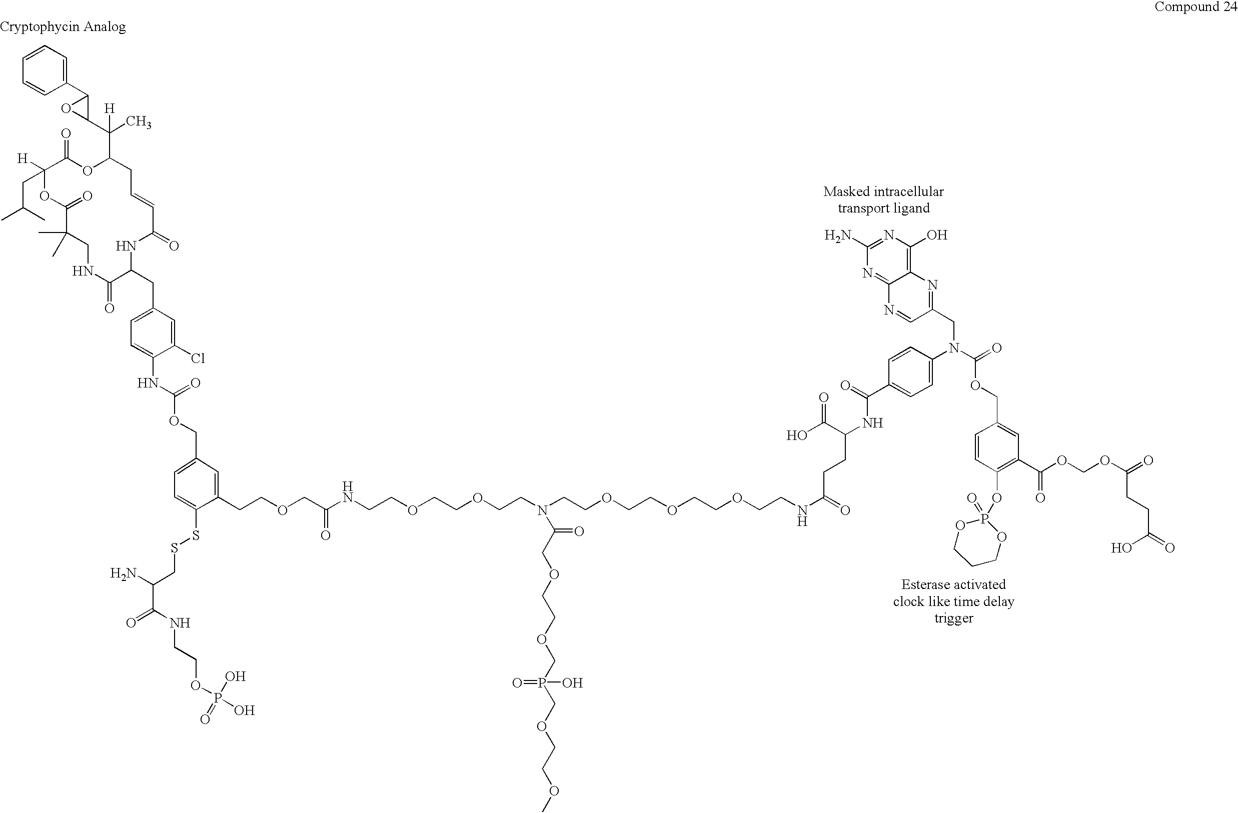

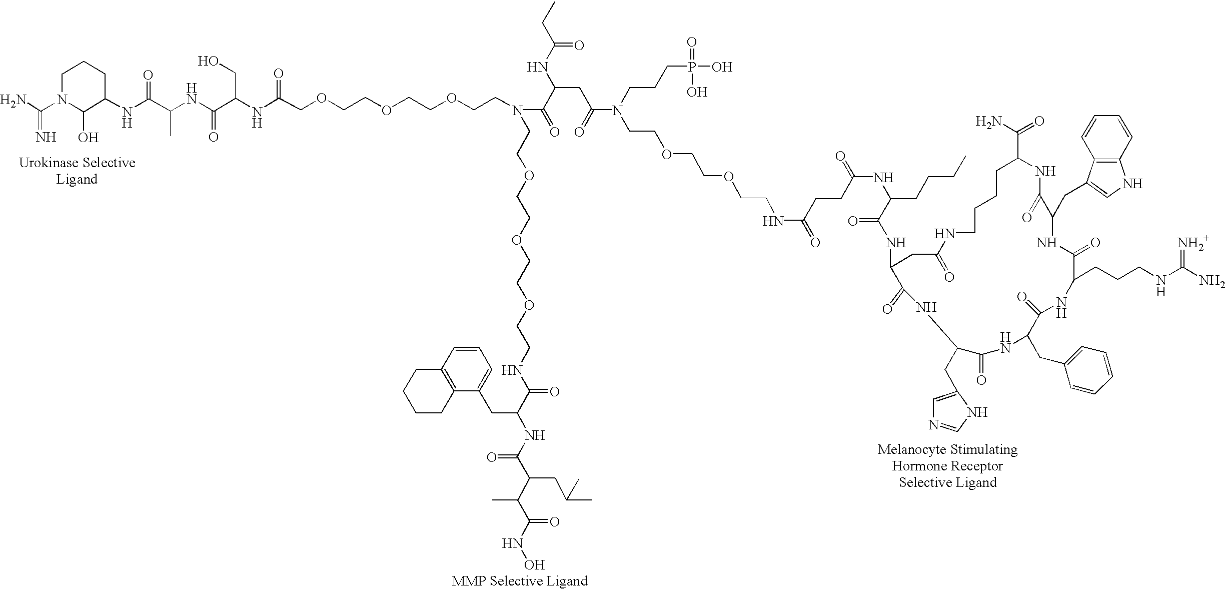

Compound 24 may be prepared by replacing compound 23.1 with compound 24.1 in the method described for the synthesis of compound 23. Also in this example, final deprotection should include an additional treatment with dilute acid to remove the 1-methyl-1-(4-biphenylyl)ethyl carbamate (Bpoc).

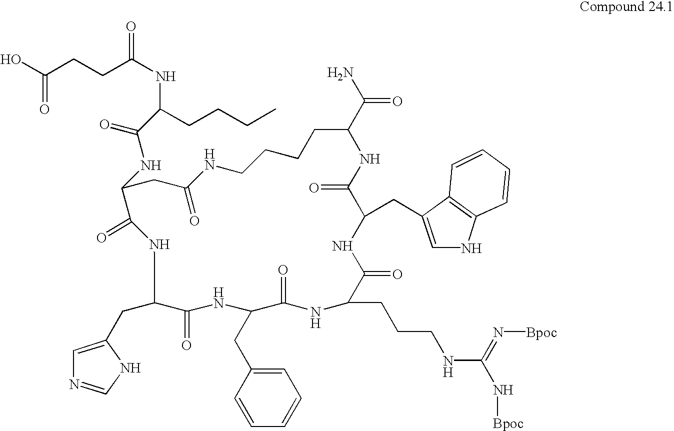

Compound 24.1 may be prepared using routine methods of peptide synthesis. The phenylalanine residue has the D configuration. The other amino acids have the L-configuration. The following references relate to this subject matter: Haskell-Luevano C., et al., “Biological and Conformational Examination of Stereochemical Modifications Using the Template Melanotropin Peptide, Ac-Nle-c[Asp-His-Phe-Arg-Trp-Ala-Lys]-NH₂, on Human Melanocortin Receptors,” J Med Chem, 40:1738-1748 (1997); Bednarek M. A., et al., “Structure-function Studies on the Cyclic Peptide MT-II, Lactam Derivative of α-melanotropin,” Peptides, 20:401-409 (1999), the contents of which are incorporated herein by reference in their entirety.

### Example 25

Compound 25 is similar to compound 24 except that a different melanocyte stimulating hormone receptor selective ligand is employed.

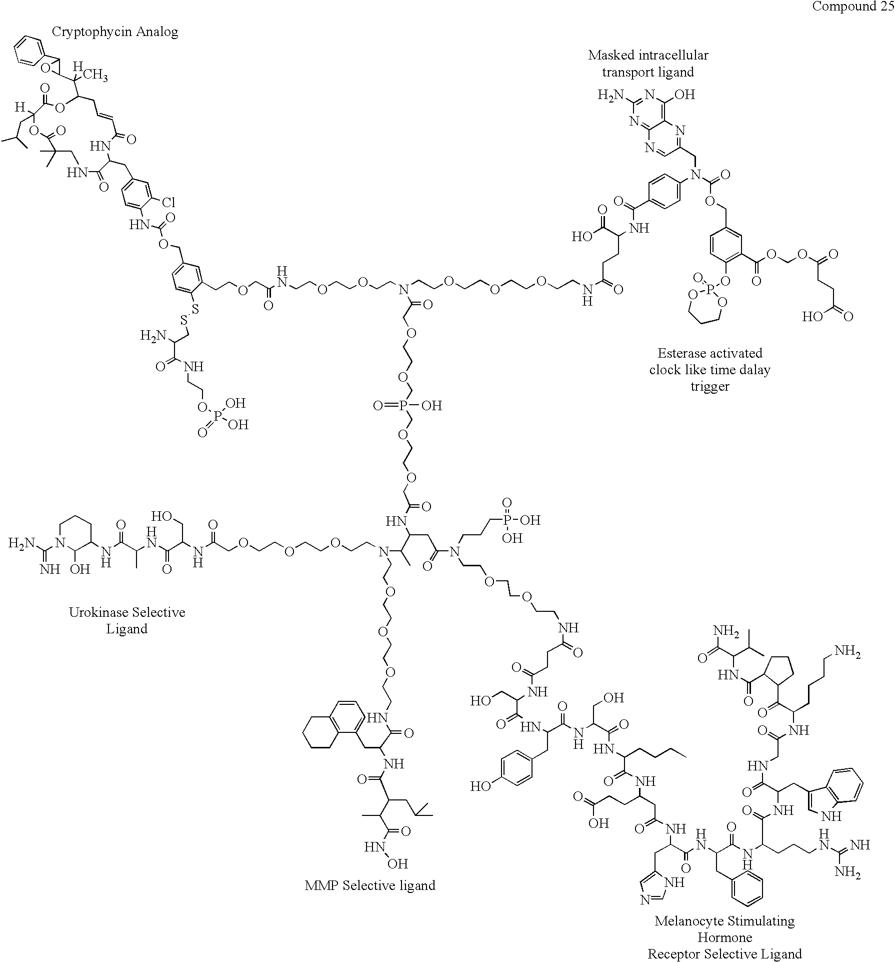

Compound 25 may be prepared as described for compound 24 replacing compound 24.1 with compound 25.1 which may be synthesized using routine techniques of peptide chemistry. The phenylalanine residue has the D configuration. The other amino acids have the L-configuration. The following references relate to this subject matter: Haskell-Luevano C., et al., “Characterizations of the Unusual Dissociation Properties of Melanotropin Peptides from the Melanocortin Receptor, hMC1R,” J Med Chem, 39:432-435 (1996); Haskell-Luevano C., et al., “Biological and Conformational Examination of Stereochemical Modifications Using the Template Melanotropin Peptide, Ac-Nle-c[Asp-His-Phe-Arg-Trp-Ala-Lys]-NH₂, on Human Melanocortin Receptors,” J Med Chem, 40:1738-1748 (1997); Bednarek M. A., et al., “Structure-function Studies on the Cyclic Peptide MT-II, Lactam Derivative of α-melanotropin,” Peptides, 20:401-409 (1999), the contents of which are incorporated herein by reference in their entirety.

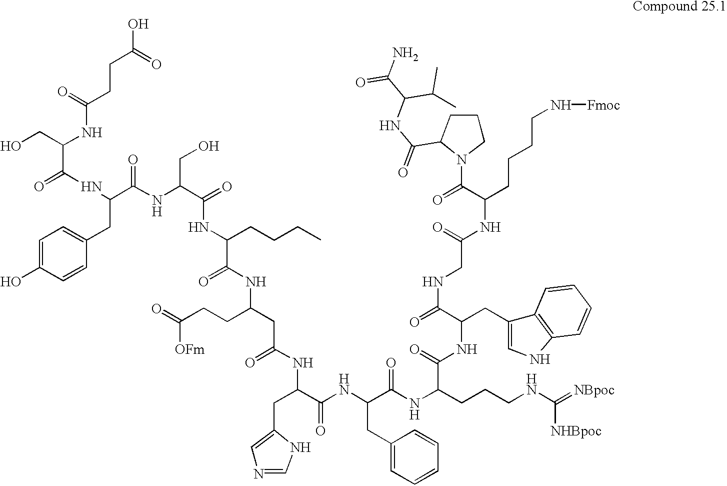

### Example 26

Compound 26 is similar to compound 23 but has targeting ligands for urokinase, matrix metalloproteinases (1, 2, 3, 9, and MT-MMP-1) and somatostatin receptor subtype2. The following references relate to this subject matter: Yang L., et al., “Synthesis and Biological Activities of Potent Peptidomimetics Selective for Somatostatin Receptor Subtype 2,” Proc Natl Acad Sci USA, 95(18):10836-41 (1998), the contents of which are incorporated herein by reference in their entirety.

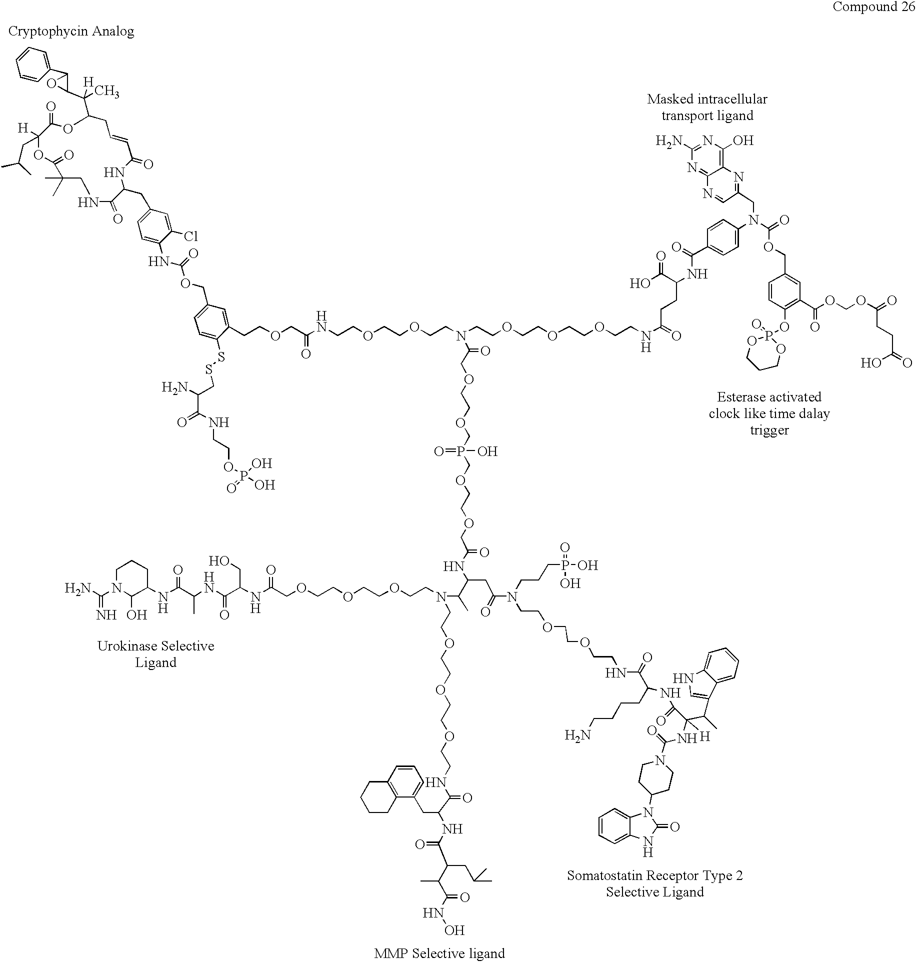

Compound 26 may be prepared by substituting compound 26.1 for compound 24.1 in the method described for the preparation of compound 24.

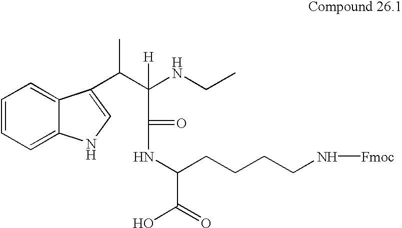

Compound 26.1 may be prepared by treating the corresponding t-butyl ester with 9H-fluoren-9-ylmethyl chloroformate in the presence of a base such as pyridine in an inert solvent followed by treatment with trifluoroacetic acid to cleave the t-butyl ester. The t-butyl ester derivative is a known compound. The following references relate to this subject matter: Yang L., et al., “Synthesis and Biological Activities of Potent Peptidomimetics Selective for Somatostatin Receptor Subtype 2,” Proc Natl Acad Sci USA, 95(18):10836-41 (1998), the contents of which are incorporated herein by reference in their entirety.

### Example 27

Compound 27 is similar to compound 26, however a different somatostatin receptor selective ligand (Octreotide) is employed which binds with high affinity to SSTR2b, 3, 4,and 5. The following references relate to this subject matter: U.S. Pat. No. 4,395,403 Jul. 26, 1983 Bauer, et al., “Polypeptides, Processes for their Production, Pharmaceutical Compositions Comprising Said Polypeptides and their Use”, the contents of which are incorporated herein by reference in their entirety.

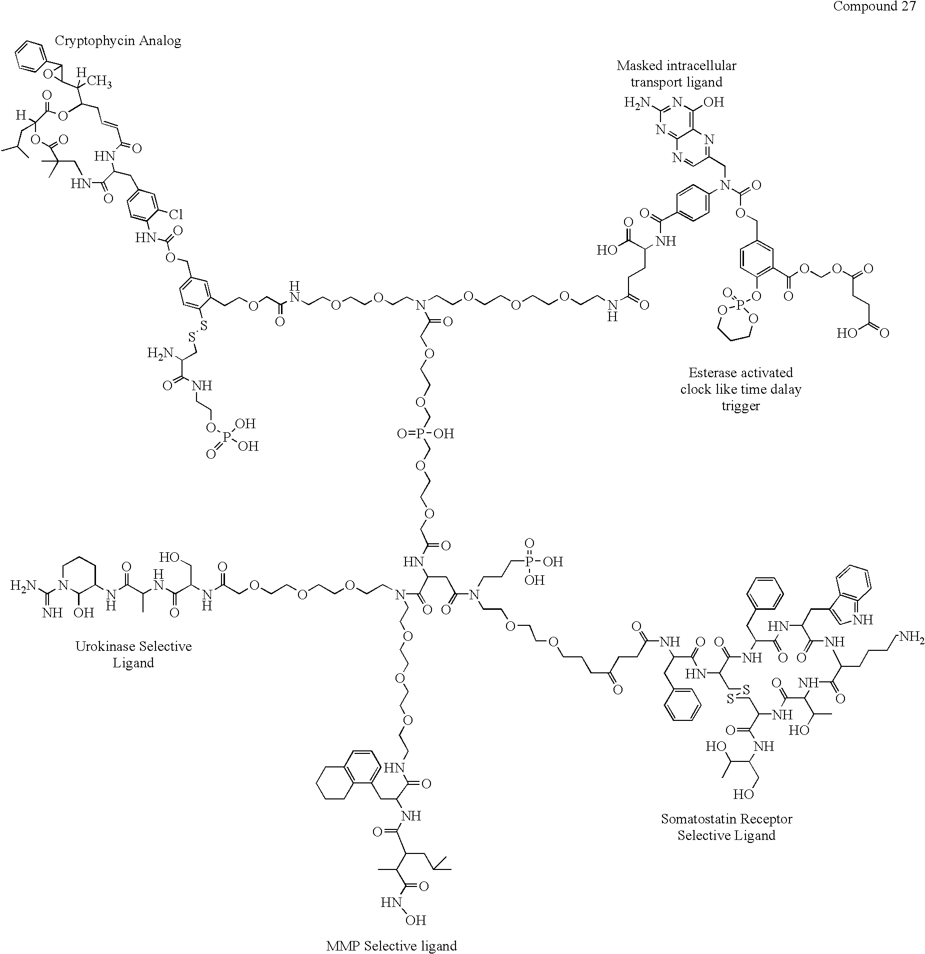

Compound 27 may be prepared by the procedures described for compound 24 by substituting compound 27.1 for compound 24.1 in the synthetic scheme.

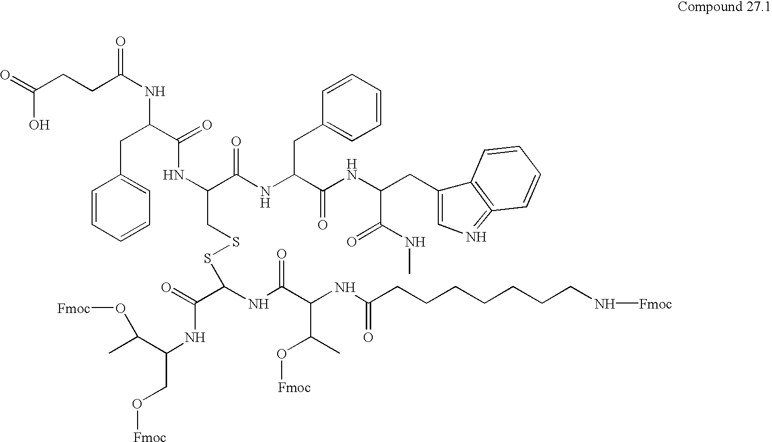

### Example 28

Compound 28 has targeting ligands for Cathepsin B, urokinase, matrix metalloproteinases (1, 2, 3, 9, and MT-MMP-1). The drug has a masked folic acid as an intracellular transport ligand, and will release a cryptophycin analog upon activation of an intracellular trigger by thioreductase. The cathepsin B ligand will irreversibly bind to the enzyme and in the process will create neoantigens. The patient may be sensitized to these neoantigens to evoke a targeted immune response. Accordingly, this drug will provide dual mechanisms of tumor destruction: direct killing by the potent cryptophycin analog and indirect killing by an intense immune response against the neoantigens. The following references relate to this subject matter: Matsumoto K., et al., “X-Ray Crystal Structure of Papain Complexed with Cathepsin B-specific Covalent-type Inhibitor: Substrate Specificity and Inhibitory Activity,” Biochim Biophys Acta, 1383:93-100 (1998); Towatari T., et al., “Novel Epoxysuccinyl Peptides. A Selective Inhibitor of Cathepsin B, in Vivo,” FEBS, 280(2):311-315 (1991); Yamamoto A., et al., “Binding Mode of CA074, a Specific Irreversible Inhibitor, to Bovine Cathepsin B as Determined by X-Ray Crystal Analysis of the Complex,” J Biochem, 121:974-977 (1997); Gour-Salin B. J., et al., “Epoxysuccinyl Dipeptides as Selective Inhibitors of Cathepsin B,” J Med Chem, 36:720-725 (1993), the contents of which are incorporated herein by reference in their entirety.

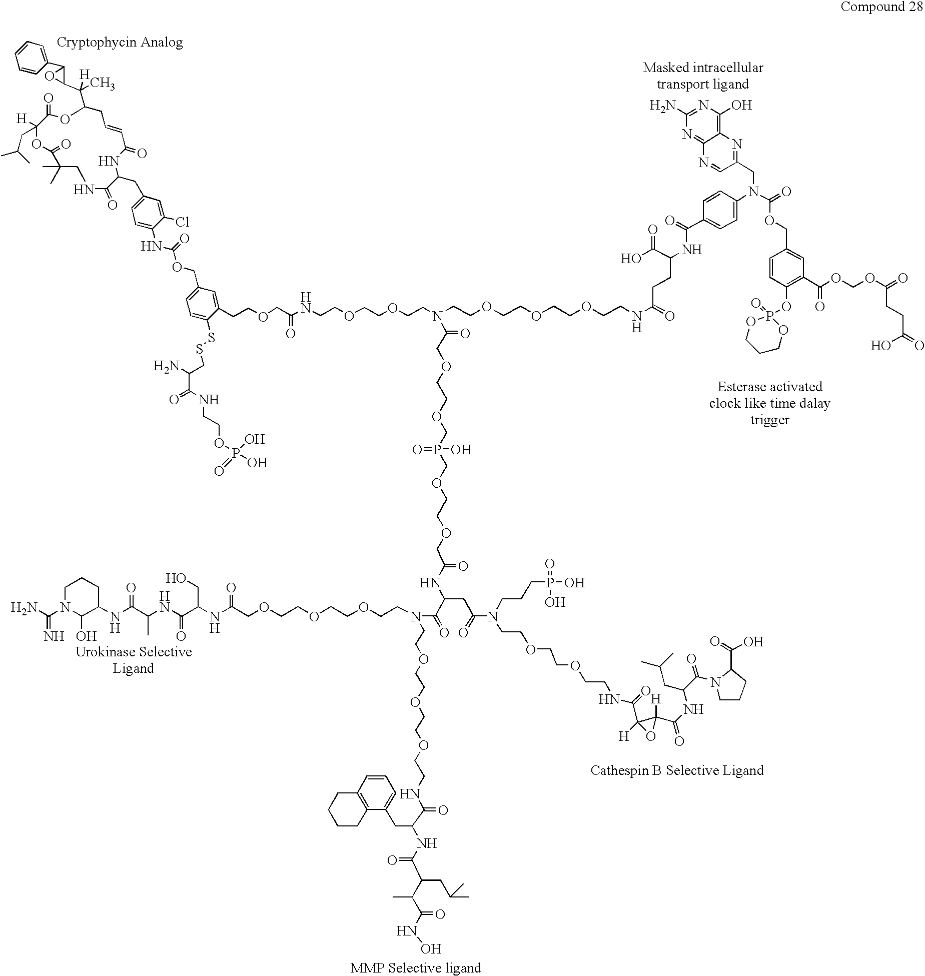

Compound 28 may be prepared by methods described for compound 24 by substituting compound 28.1 for compound 24.1 in the synthesis.

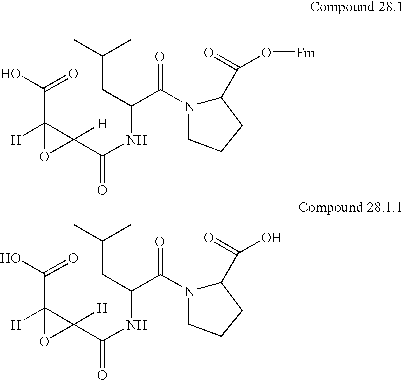

Compound 28.1 may be prepared from the known compound 28.2. The following references relate to this subject matter: Gour-Salin B. J., et al., “Epoxysuccinyl Dipeptides as Selective Inhibitors of Cathepsin B,” J Med Chem, 36:720-725 (1993), the contents of which are incorporated herein by reference in their entirety.

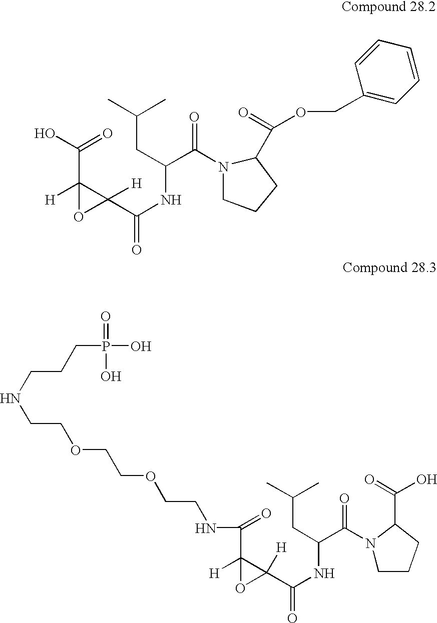

Esterification of compound 28.2 with (1,1-Dioxo-1H-1λ6-benzo[b]thiophen-2-yl)-methanol and dicyclohexylcarbodiimide, followed by catalytic hydrogenation with palladium on carbon to remove the benzyl group, followed by esterification with (9H-Fluoren-9-yl)-methanol and dicyclohexylcarbodiimide, followed by selective cleavage of the Bsm ester with tris(2-aminoethyl)-amine will give compound 28.1.

The neoantigens (and neoantigen precursors) to be used to sensitize patients in the method of targeted immunotherapy with compound 28 may be prepared by incubating human cathepsin B with a compound such as compound 28.3 or compound 28.1.1. Alternatively, synthetic oligopeptides containing approximately 7-20 amino acids corresponding to the amino acid sequence of cathepsin B that contain the cysteine alkylated by the epoxide may be prepared and alkylated with compound 28.3 or compound 28.1.

### Example 29

Compound 29 is similar to compound 28, however the linker to the Cathepsin B selective ligand has a disulfide bond that may provide a more facile catabolic route and facilitate neoantigen formation.

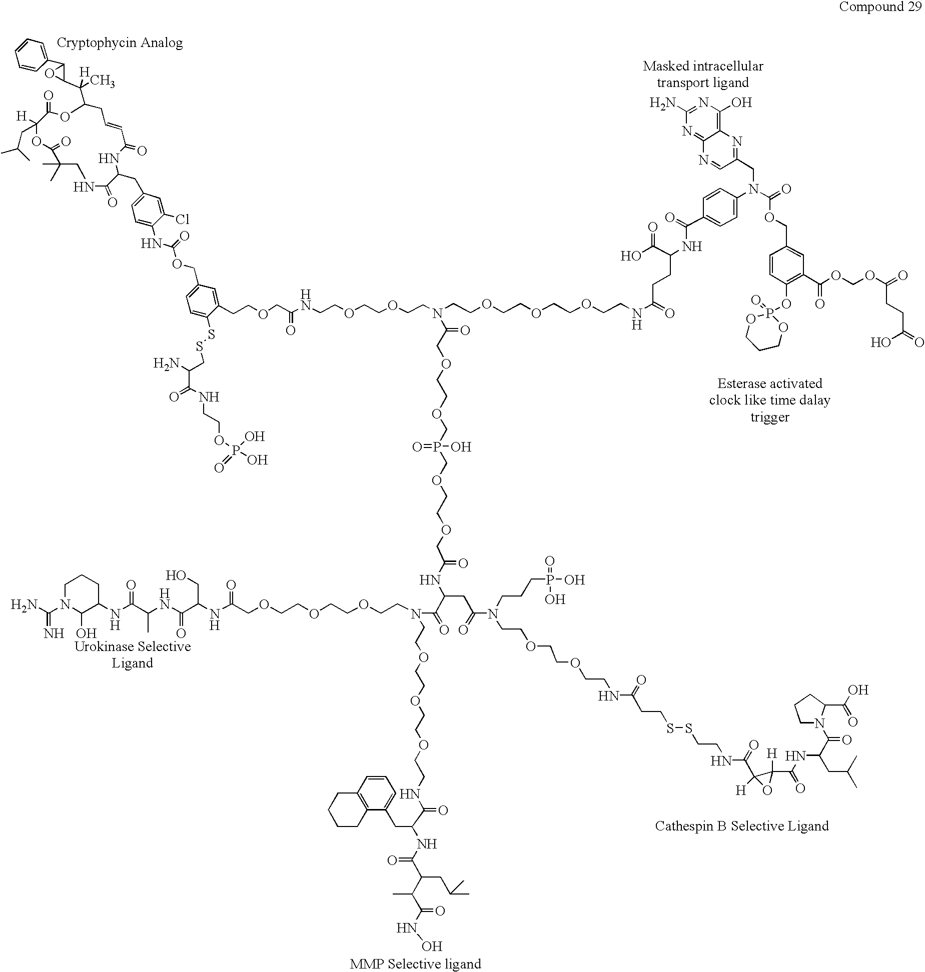

Compound 29 may be prepared by the methods for compound 28 by substituting compound 29.1 for compound 28.1.

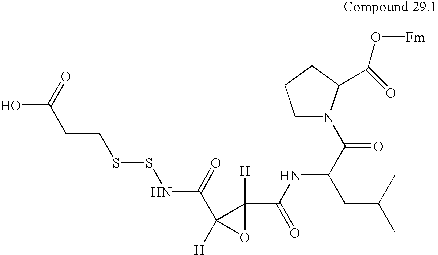

Compound 29.1 may be prepared by reacting compound 28.1 with N-hydroxysuccinimide and dicyclohexylcarbodiimide in an inert solvent and then reacting this active ester with 3-(2-Amino-ethyidisulfanyl)-propionic acid and base in an inert solvent.

The neoantigens (and neoantigen precursors), to be employed for sensitization for use with the method of targeted neoantigen immunotherapy with compound 29, may be prepared in an analogous manner as described for compound 28.

### Examples 30a, 30b and 30c

Compounds 30a 30b and 30c are similar to compound 29, however different clock like time delayed triggers are employed to unmask the intracellular transport ligand. Ortho positioned electron donating groups promote elimination of benzylic compounds at rates that are slower than the corresponding para derivatives and provide for a time delay clock like trigger. For example, under conditions in which para thio-benzyl carbamates undergo elimination with a half life of 10 minutes the corresponding ortho derivative has a half life of 72 min. Similar behavior is expected for ortho hydroxy, and ortho amino benzylic derivatives. The following references relate to this subject matter: Senter, Peter D., et al., “Development of a Drug-Release Strategy Based on the Reductive Fragmentation of Benzyl Carbamate Disulfides,” J Org Chem, 55:2975-2978 (1990), the contents of which are incorporated herein by reference in their entirety.

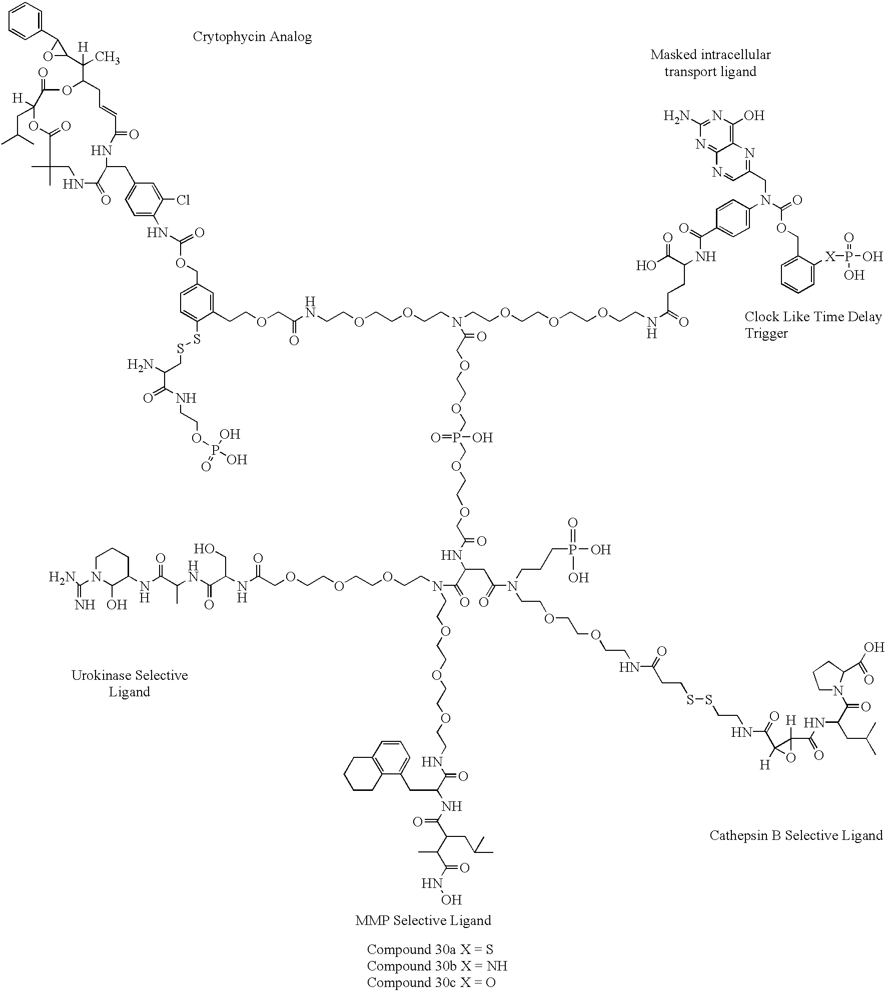

Compounds 30a, 30b, and 30c may be prepared by substituting compound 30a.1, 30b.1 and 30c.1 respectively for compound 6.2.0b in the procedure described for compound 29.

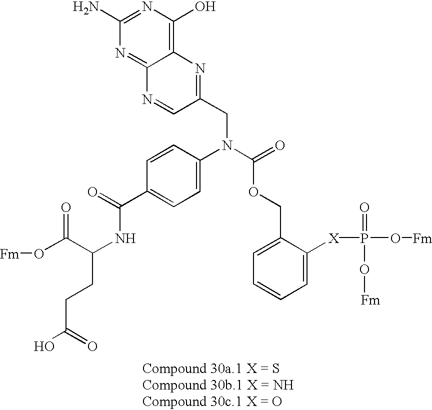

Compound 30a.1, 30b.1 and 30c.1 may be prepared by reacting compounds 30a.2, 30b.2 and 30c.2 respectively with compound 30.3 in an inert solvent in the presence of a base such as pyridine, and then cleaving the 2,2,2 trichloroethyl ester with Zn and phosphate buffer.

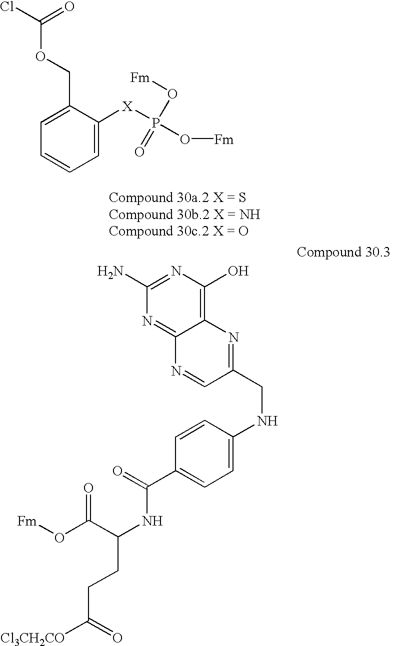

Compounds 30a.2, 30b.2, and 30c.2 may be prepared by treating the corresponding benzylic alcohol compounds 30a.3, 30b.3 and 30c.3 with phosgene in an inert solvent.

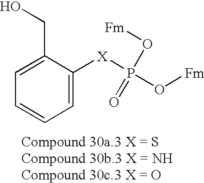

Compound 30.a.3 may be prepared by treating 2-trityloxymethyl-benzenethiol with phosphorochloridic acid bis-(9H-fluoren-9-ylmethyl) ester and base in an inert solvent followed by acid treatment.

The compound 2-trityloxymethyl-benzenethiol may be prepared by reacting [2-(2-Hydroxymethyl-phenyldisulfanyl)-phenyl]-methanol with trityl chloride and base in an inert solvent and then reducing the disulfide bond with a reagent such as sodium borohydride.

Compound 30.b.3 may be prepared by treating 2-aminobenzyl alcohol with chlorotrimethylsilane and base, and then reacting with phosphorochloridic acid bis-(9H-fluoren-9-ylmethyl) ester and base in an inert solvent, followed by hydrolysis of the silyl groups.

Compound 30c.3 may be prepared by treating 2-Hydroxy-benzaldehyde with phosphorochloridic acid bis-(9H-fluoren-9-ylmethyl) ester and base in an inert solvent, followed by reduction of the aldehyde to the alcohol. The reduction may be carried out by selective hydrogenation with palladium on carbon or by a reagent such as sodium borohydride.

Compound 30.3 may be prepared by a multi-step procedure. Compound 20.8.3c and compound 30.3a may be coupled. Treating the product with acid will remove the t-Boc group and give compound 30.3.

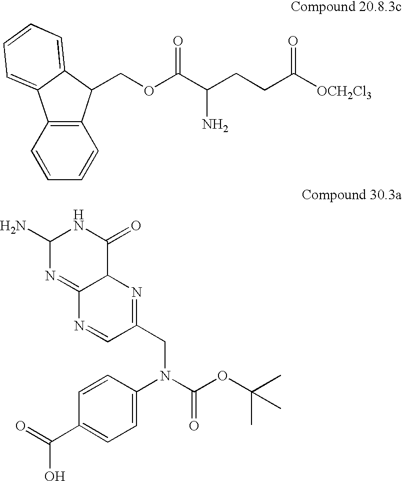

Compound 30.3a may be prepared by treating pteroic acid with a reagent such as di-t-butyl pyrocarbonate in an inert solvent.

In an alternate method for the preparation of compound 30.3, pteroic acid may be treated with an excess of a reagent such as hexamethyldisilazane and a catalytic amount of chlorotrimethylsilane in an inert solvent. The silylated derivative may then be reacted with (1,1-Dioxo-1H-1λ6-benzo[b]thiophen-2-yl)-methyl chloroformate in an inert solvent. After hydrolysis of the silyl groups, the product may be coupled to compound 20.8.3c. Selective removal of the Bsmoc group with tris(2-aminoethyl)-amine will give compound 30.3.

### Example 31

Compound 31 is similar to compound 30 but a different cyclization based phosphatase activated time delayed clock like trigger is employed to unmask the intracellular transport ligand. Phosphatase will cleave the phosphoester and formaldehyde will be eliminated, thereby converting the nonnucleophilic quarternary ammonium group into a nucleophilic tertiary amino group. The tertiary amino group will then catalyze the hydrolysis of the carbamate by a cyclic intermediate with a half life of approximately 40 minutes. The following references relate to this subject matter: Saari W. S., et al., “Cyclization-Activated Prodrugs. Basic Carbamates of 4-Hydroxyanisole,” J Med Chem, 33:97-101 (1990); Krise J. P., et al., “Novel Prodrug Approach for Tertiary Amines: Synthesis and Preliminary Evaluation of N-Phosphonooxymethyl Prodrugs,” J Med Chem, 42:3094-3100 (1999); Krise J. P., et al., “A Novel Prodrug Approach for Tertiary Amines. 3. In Vivo Evaluation of Two N-Phosphonooxymethyl Prodrugs in Rats and Dogs,” J Pharm Sciences, 88(9):928-932 (1999), the contents of which are incorporated herein by reference in their entirety.

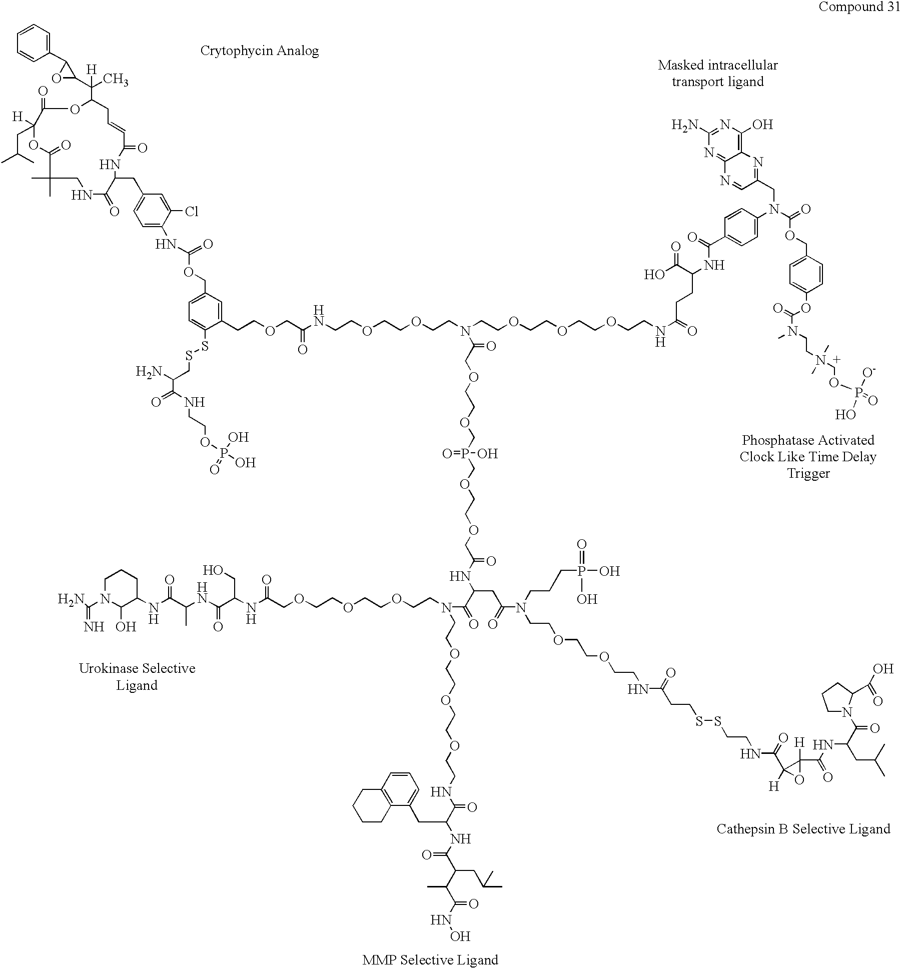

Compound 31 may be prepared by substituting compound 31.1 for compound 30c.1 in the process described for the synthesis of compound 30c.

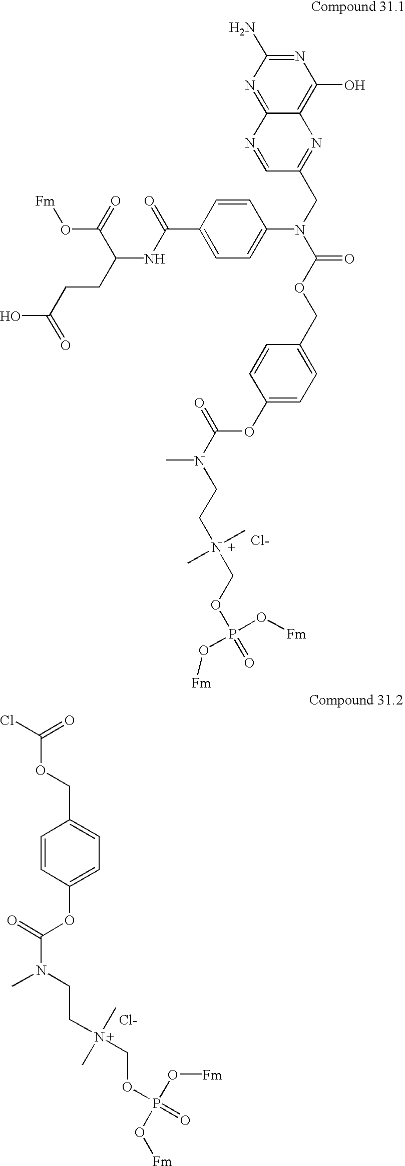

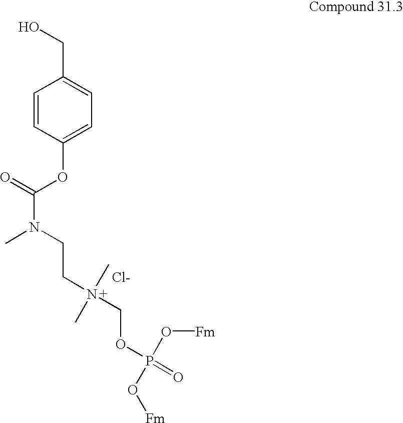

Compound 31.1 may be prepared by reacting compound 31.2 and compound 30.3 and then cleaving the 2,2,2 trichloroethyl ester with Zn. Compound 31.2 may be prepared by reacting compound 31.3 with phosgene in an inert solvent. Compound 31.3 may be prepared by reduction of the corresponding aldehyde (compound 31.4) with hydrogen and palladium on carbon or with a reagent such as sodium borohyd ride. Compound 31.4 may be prepared by reacting p-hydroxybenzaldehyde with phosgene in an inert solvent and then reacting the resulting chloforomate with compound 31.5 in an inert solvent in the presence of a base such as pyridine.

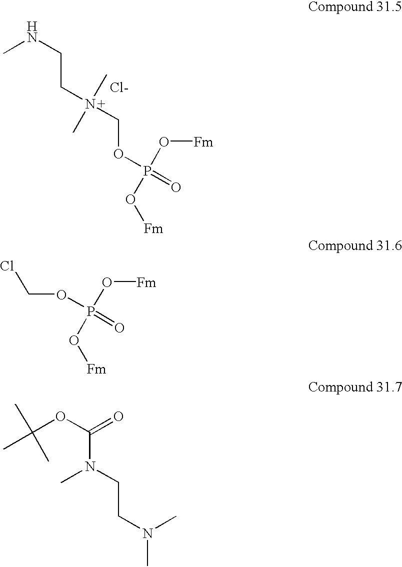

Compound 31.5 may be prepared by reacting compounds 31.6 and 31.7 in an inert solvent and then treating with acid to remove the t-Boc group. Compound 31.6 may be prepared by alkylating tetramethyl-ammonium bis-(9H-fluoren-9-ylmethyl) phosphate with chloroiodomethane in an inert solvent. Compound 31.7 may be prepared by treating N,N,N′-trimethyl-ethane-1,2-diamine with di-t-butyl pyrocarbonate and in an inert solvent. The following references relate to this subject matter: Saari W. S., et al., “Cyclization-Activated Prodrugs. Basic Carbamates of 4-Hydroxyanisole,” J Med Chem, 33:97-101 (1990); Krise J. P., et al., “Novel Prodrug Approach for Tertiary Amines: Synthesis and Preliminary Evaluation of N-Phosphonooxymethyl Prodrugs,” J Med Chem, 42:3094-3100 (1999); Krise J. P., et al., “A Novel Prodrug Approach for Tertiary Amines. 3. In Vivo Evaluation of Two N-Phosphonooxymethyl Prodrugs in Rats and Dogs,” J Pharm Sciences, 88(9):928-932 (1999), the contents of which are incorporated herein by reference in their entirety.

### Example 32

Compound 32 has targeting ligands for PSMA and laminin receptor. It has a clock like esterase activated time delayed trigger that will function to unmask the intracellular transport ligand. Esterase will unmask a carboxylate group situated ortho to the phosphotriester group. The carboxylate group will, by an intramolecular nucleophilic reaction, cleave the phosphotriester, thereby unmasking the phenolic hydroxy group. The intramolecular nucleophilic reaction is expected to proceed with a half life of approximately 90 minutes under physiological conditions. The cytotoxic agent, campothecin, will be released upon activation of an intracellular trigger. The following references relate to this subject matter: Bromilow R. H., et al., “Intramolecular Catalysis of Phosphate Triester Hydrolysis. Nucleophilic Catalysis by the Neighbouring Carboxyl Group of the Hydrolysis of Dialkyl 2-Carboxyphenyl Phosphates,” J Chem Soc, 1091-1096 (1971), the contents of which are incorporated herein by reference in their entirety.

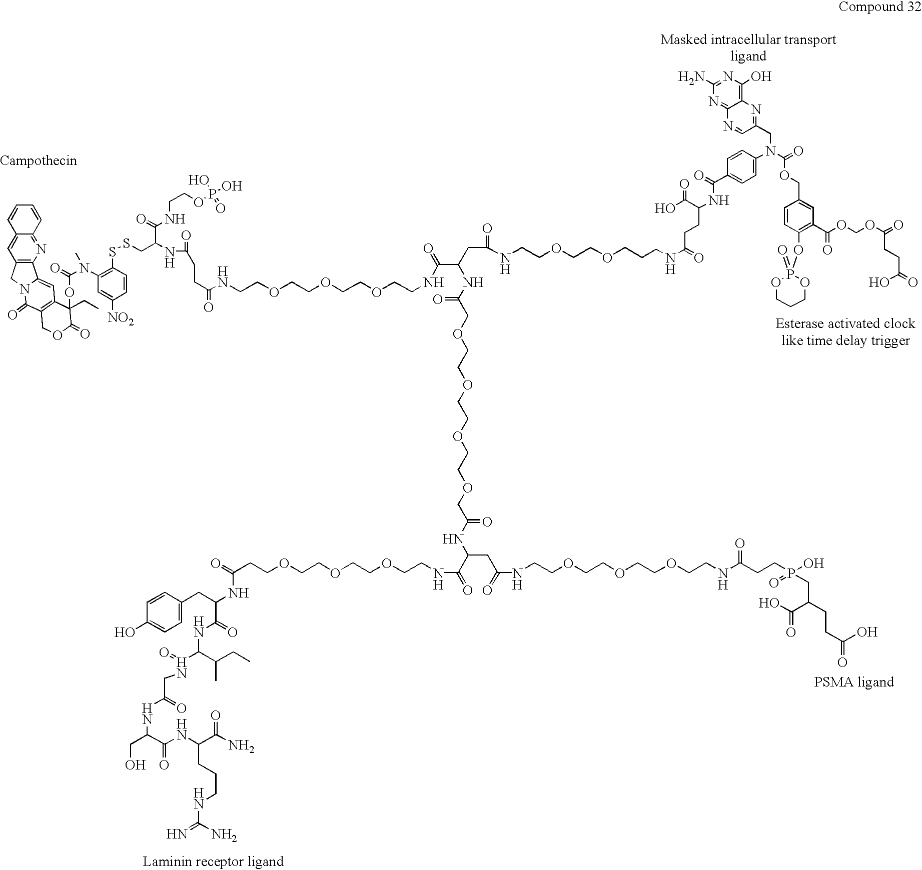

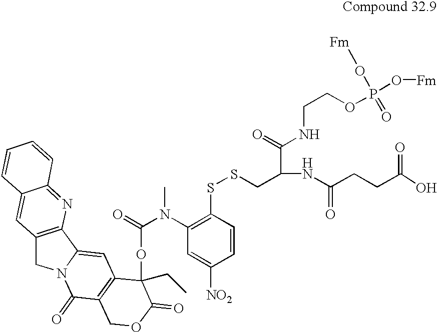

Compound 32 may be prepared by the methods described for compound 6 by replacing compound 6.2.0c with compound 32.9.

Synthesis of Compound 32.1

Compound 32.1 may be prepared by a multi-step process.

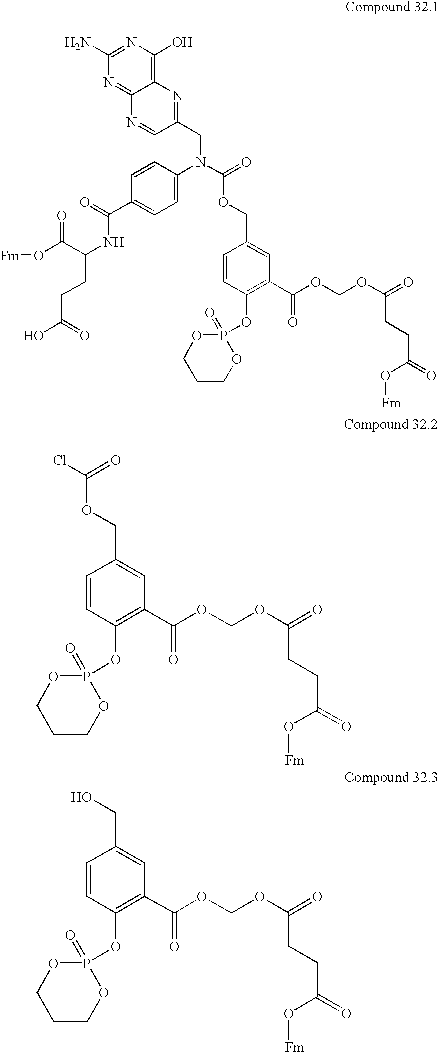

Compound 32.1 may be prepared by reacting compound 32.2 and compound 30.3 in an inert solvent in the presence of a base such as pyridine and then cleaving the 2,2,2 trichloro-ethyl ester with Zn. Compound 32.2 may be prepared by treating compound 32.3 with phosgene in an inert solvent. Compound 32.3 733 may be prepared by reduction of the aldehyde compound 32.4 with hydrogen and palladium catalyst or a reagent such as sodium borohydride.

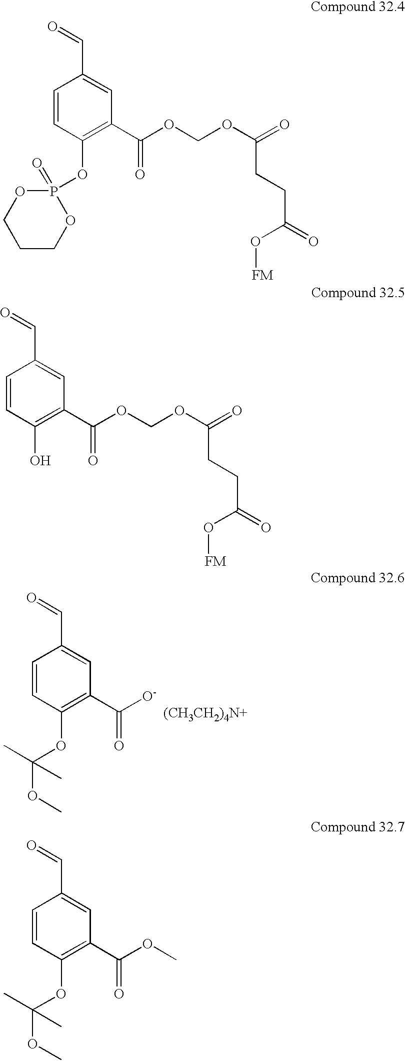

Compound 32.4 may be prepared by reacting compound 32.5 and 2-chloro-[1,3,2]dioxaphosphinane 2-oxide in an inert solvent in the presence of base. Compound 32.5 may be prepared by reacting compound 32.6 and succinic acid chloromethyl ester 9H-fluoren-9-ylmethyl ester in an inert solvent and then treating with dilute acid to selectively cleave the 1-methyl-1 methoxyethyl ether. (Alternatively, the silver salt of compound 32.6 may be employed). Succinic acid chloromethyl ester 9H-fluoren-9-ylmethyl ester may be prepared by treating succinic acid mono-(9H-fluoren-9-ylmethyl) ester with chloroiodomethane in an inert solvent.

Synthesis of Compound 32.9

Compound 32.9 may be prepared by reacting compound 32.10 and compound 32.11 in an inert solvent in the presence of a base such as pyridine and then selectively removing the Bsmoc group with tris(2-aminoethyl)-amine, and then reacting with succinic anhydride in an inert solvent.

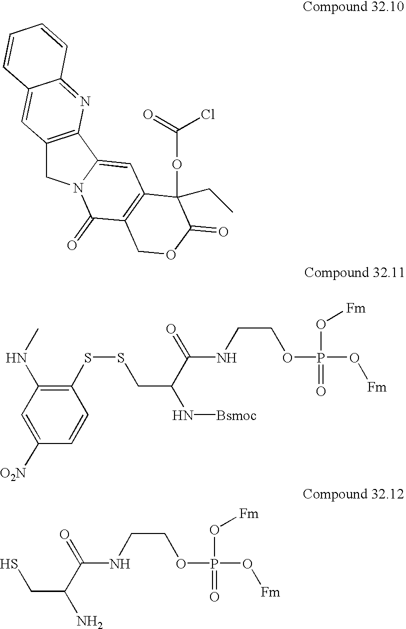

Compound 32.10 may be prepared by treating campothecan with phosgene in an inert solvent.

Compound 32.11 may be prepared by a multi-step process. Reacting (2-Mercapto-5-nitro-phenyl)-methyl-carbamic acid tert-butyl ester with diethyl azidocarboxylate in an inert solvent and then reacting the product with compound 32.12 will give compound 32.13. Treatment with (1,1-Dioxo-1H-1λ6-benzo[b]thiophen-2-yl)-methyl chloroformate and base in an inert solvent, followed by treatment with acid to cleave the t-Boc group, will give compound

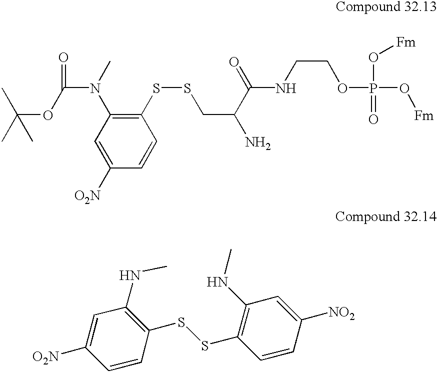

The compound (2-Mercapto-5-nitro-phenyl)-methyl-carbamic acid tert-butyl ester may be prepared by treating compound 32.14 with di-t-butyl pyrocarbonate in an inert solvent and then reducing the disulfide bond with a reagent such as sodium borohydride.

### Example 33

Compound 33 is similar to compound 6, however compound 33 has a different type of esterase activated time delay clock like trigger, which will unmask the intracellular transport ligand. The esters of N,N-disubstituted hydroxyacetamides are very rapidly cleaved by esterase. The triggering mechanisms are similar to those described for compound 32. The following references relate to this subject matter: Bundgaard H.; Nielsen N. M., “Esters of N,N-Disubstituted 2-Hydroxyacetamides as a Novel Highly Biolabile Prodrug Type for Carboxylic Acid Agents,” J Med Chem, 30(3):450-453 (1987), the contents of which are incorporated herein by reference in their entirety.

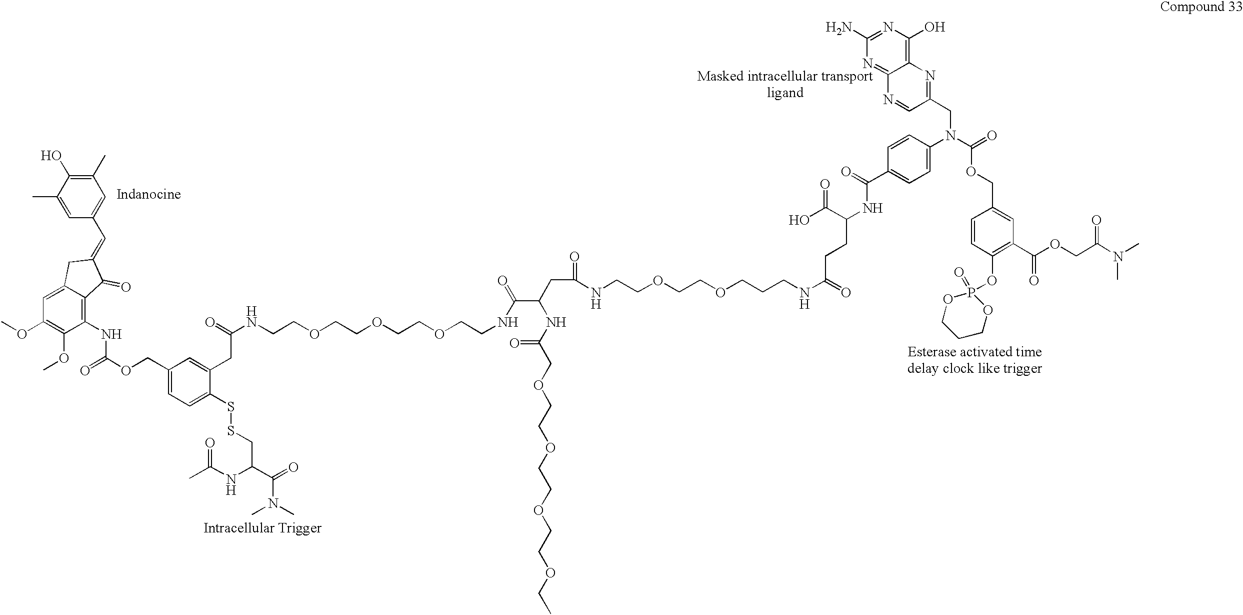

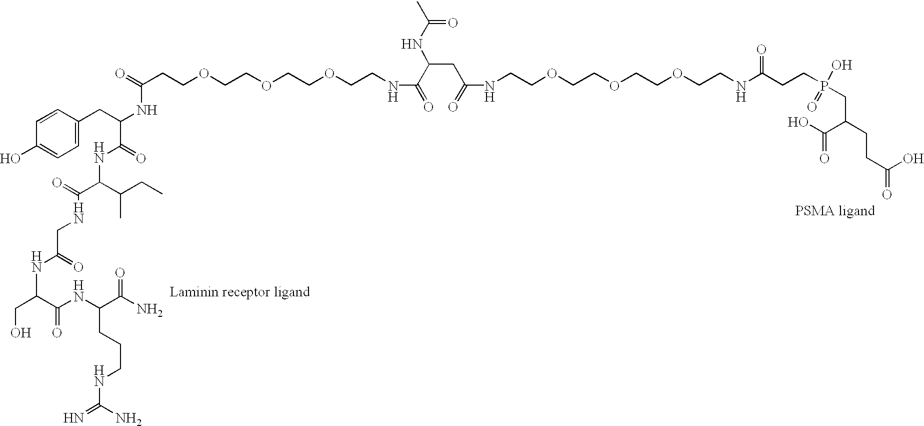

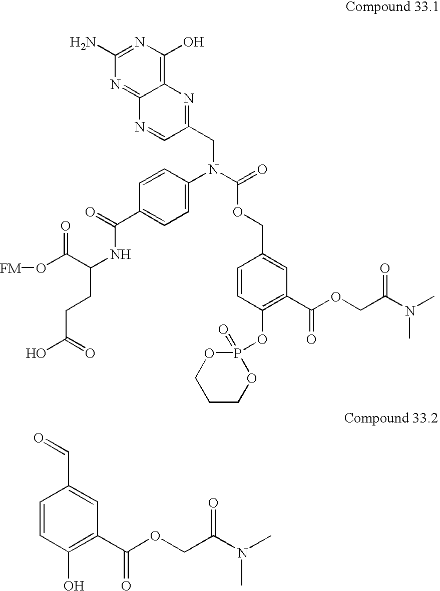

Compound 33 may be prepared by replacing compound 6.2.0b with compound 33.1 in the procedure described for the synthesis of compound 6. Compound 33.1 may be made by the procedure described for compound 32.1 by substituting compound 33.2 for compound 32.5. Compound 33.2 may be prepared by coupling compound 32.6 and 2-hydroxy-N,N-dimethyl-acetamide and then treating with acid to cleave the 1-methyl-1 methoxyethyl ether.

### Example 34

Compound 34 is a multifunctional drug delivery vehicle for use in the method of targeted neoantigen immunotherapy against epidermal growth factor receptors. It has targeting ligands for sigma receptors, urokinase, matrix metalloproteinases (1, 2, 3, 9, and MT-MMP-1) and epidermal growth factor receptors including erB2. The drug has a masked folic acid as an intracellular transport ligand with a phosphatase activated time delay clock like trigger. Thioreductase will activate an intracellular trigger and release a compound that will irreversibly bind to the epidermal growth factor receptors and generate neoantigen precursors. The patient may be sensitized to these epidermal growth factor based neoantigens to 737 evoke a targeted immune response against the tumor. The following references relate to this subject matter: Discafani C. M., et al., “Irreversible Inhibition of Epidermal Growth Factor Receptor Tyrosine Kinase with In Vivo Activity by N-[4-[(3-Bromophenyl)amino]-6-quinazolinyl]-2-butynamide (CL-387,785),” Biochem Pharm, 57:917-925 (1999); Smaill J. B., et al., “Tyrosine Kinase Inhibitors. 17. Irreversible Inhibitors of the Epidermal Growth Factor Receptor: 4-(Phenylamino)quinazoline- and 4-(Phenylamino)pyrido[3,2-d]pyrimidine-6-acrylamides Bearing Additional Solubilizing Functions,” J Med Chem, 43:1380-1397 (2000), the contents of which are incorporated herein by reference in their entirety.

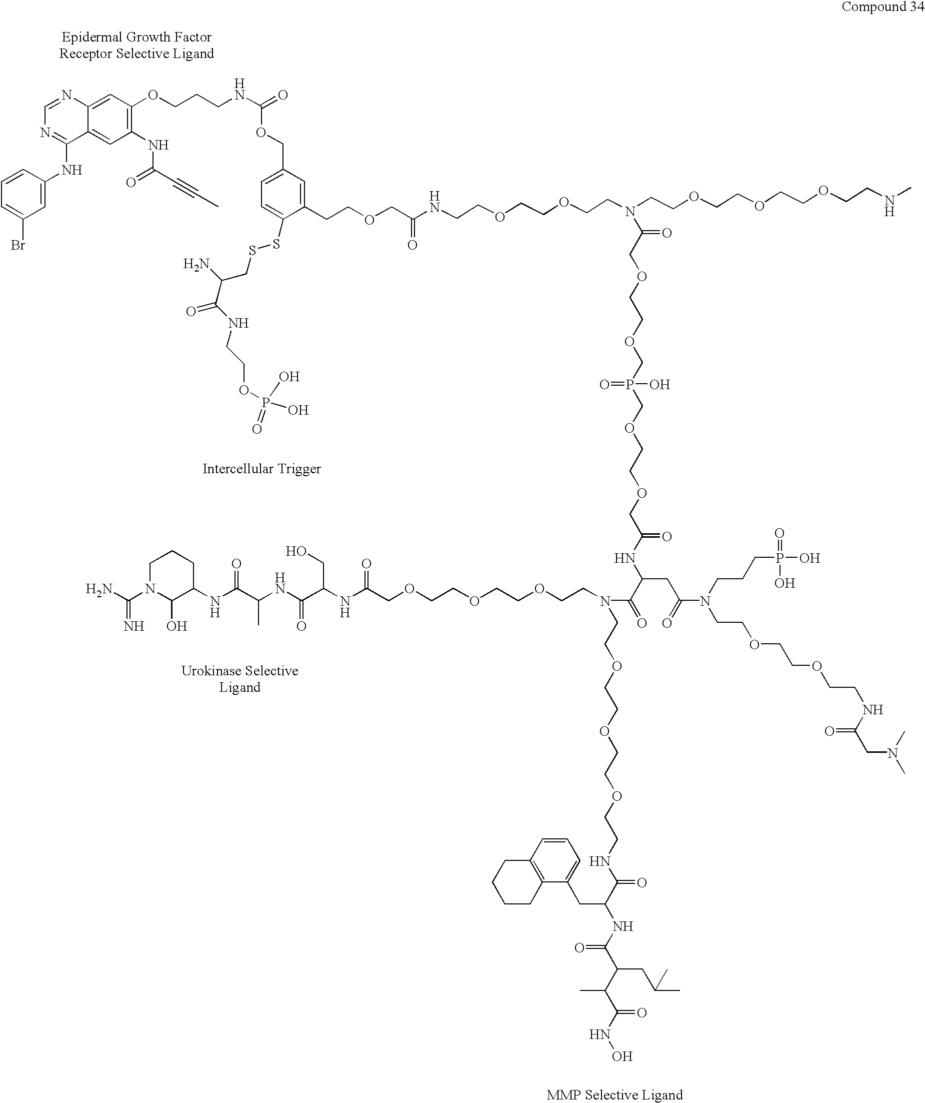

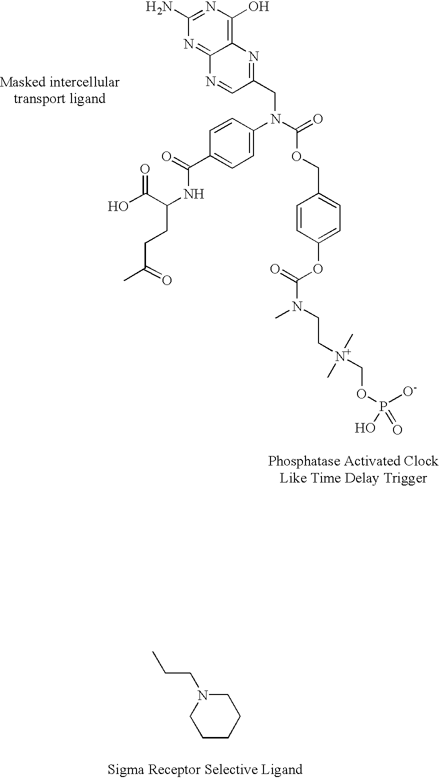

Compound 34 may be prepared by the methods described for the synthesis of compound 21 by replacing compound 21.2.2e with compound 14.5, then replacing compound 21.1.2 with compound 34.1 and also replacing compound 6.2.0b with compound 31.1.

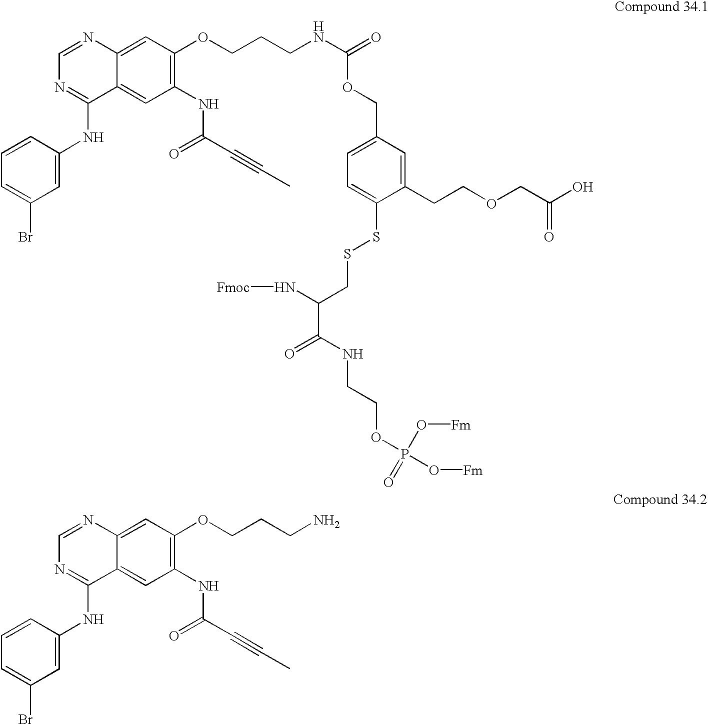

Compound 34.1 may be prepared by reacting compound 34.2 and compound 23.2b in an inert solvent in the presence of a base such as pyridine and then treating with tris(2-aminoethyl)-amine to cleave the Bsm ester.

Compound 34.2 may be prepared by a multi-step process. Reacting (3-bromo-phenyl)-(7-fluoro-6-nitro-quinazolin-4-yl)-amine and 2-(3-Hydroxy-propyl)-isoindole-1,3-dione in the presence of a strong base such as sodium hydride in an inert solvent will give compound 34.3.

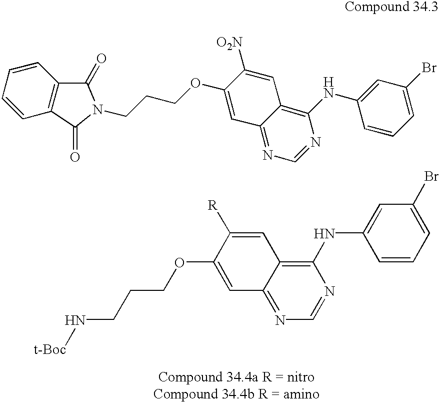

Removal of the phthalyl protecting group, followed by treatment with di-t-butyl pyrocarbonate and in an inert solvent will give compound 34.4a. Suitable reagents that are compatible with the nitro group to accomplish the deprotection are well known. The following references relate to this subject matter: Greene, Theodora W.; Wuts, Peter G. M. (1999) “Protective Groups in Organic Synthesis” John Wiley & Sons, Inc. p 565, the contents of which are incorporated herein by reference in their entirety.

Catalytic hydrogenation of the nitro group with palladium on carbon will give compound 34.4b. Treatment with the mixed anhydride formed between but-2-ynoic acid and isobutyl chloroformate in an inert solvent in the presence of base, followed by treatment with acid to remove the t-Boc group will give compound 34.2. The following references relate to this subject matter: Discafani C. M., et al., “Irreversible Inhibition of Epidermal Growth Factor Receptor Tyrosine Kinase with In Vivo Activity by N-[4-[(3-Bromophenyl)amino]-6-quinazolinyl]-2-butynamide (CL-387,785),” Biochem Pharm, 57:917-925 (1999); Smaill J. B., et al., “Tyrosine Kinase Inhibitors. 17. Irreversible Inhibitors of the Epidermal Growth Factor Receptor: 4-(Phenylamino)quinazoline- and 4-(Phenylamino)pyrido[3,2-d]pyrimidine-6-acrylamides Bearing Additional Solubilizing Functions,” J Med Chem, 43:1380-1397 (2000); U.S. Pat. No. 5,760,04, Jun. 2, 1998, Wissner et al., “4-Aminoquinazoline EGFR Inhibitors”, the contents of which are incorporated herein by reference in their entirety. Patients may be sensitized to the epidermal growth factor receptor or erb2 derived neoantigens by immunizing with epidermal growth factor receptors or erb2 that has been reacted with compound 34.2. Alternatively, synthetic oligopeptides that correspond to the amino acid sequence of the receptor with compound 34.2 covalently attached may be employed.

### Example 35

Compound 35 is similar to compound 34, however a different group is employed to modify the epidermal growth factor receptor. The following references relate to this subject matter: Smaill J. B., et al., “Tyrosine Kinase Inhibitors. 17. Irreversible Inhibitors of the Epidermal Growth Factor Receptor: 4-(Phenylamino)quinazoline- and 4-(Phenylamino)pyrido[3,2-d]pyrimidine-6-acrylamides Bearing Additional Solubilizing Functions,” J Med Chem, 43:1380-1397 (2000); Smaill J. B., et al., “Tyrosine Kinase Inhibitors. 15.4-(Phenylamino)quinazoline and 4-(Phenylamino)pyrido[d]pyrimidine Acrylamides as Irreversible Inhibitors of the ATP Binding Site of the Epidermal Growth Factor Receptor,” J Med Chem, 42:1803-1815 (1999), the contents of which are incorporated herein by reference in their entirety.

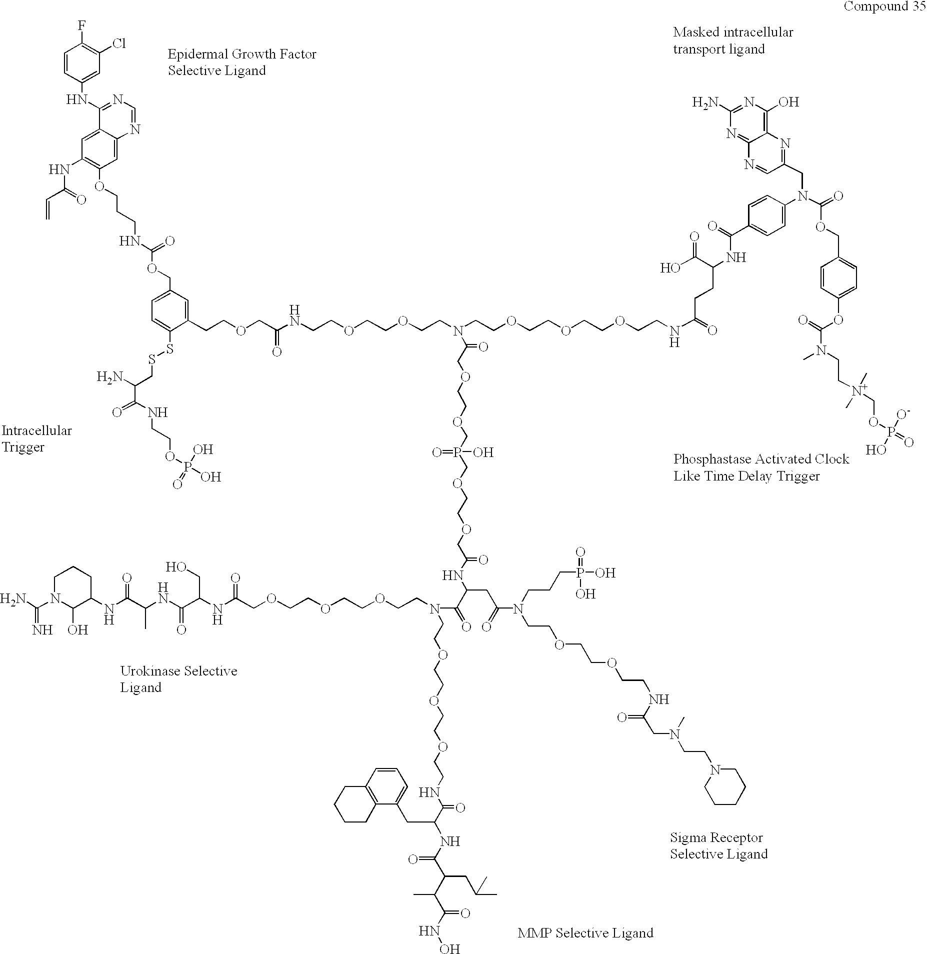

Compound 35 may be prepared by the method described for compound 34 by replacing compound 34.2 with compound 35.1.

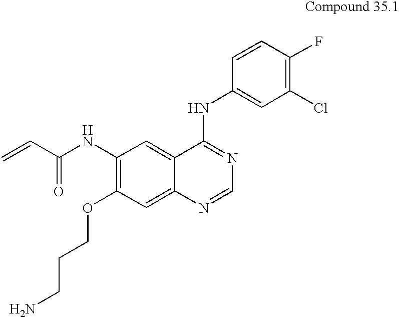

Compound 35.1 may be prepared by replacing (3-bromo-phenyl)-(7-fluoro-6-nitro-quinazolin-4-yl)-amine with (3-Chloro-4-fluoro-phenyl)-(7-fluoro-6-nitro-quinazolin-4-yl)-amine and by replacing but-2-ynoic acid with acrylic acid in the procedure described for the synthesis of compound 34.

Patients may be sensitized to the neoantigens using compounds analogous to those described in example 34.

### Example 36

Compound 36 is a multifunctional drug delivery vehicle with ligands for urokinase, matrix metalloproteinases (1, 2, 3, 9, and MT-MMP-1) and epidermal growth factor receptors including erbB2. The drug has a phosphatase activated time delay clock like trigger that will unmask the intracellular transport ligand. The drug has an intracellular trigger that when cleaved by thioreductases will free a compound that will irreversible modify epidermal growth factor receptors and erb2 receptors and in the process generate neoantigens. In addition, the drug has another intracellular trigger, which when activated will release a leukotriene receptor agonist. This leukotriene receptor agonist will, after diffusing out of the tumor cells, elicit a localized inflammatory response by activating the innate immune system. This inflammatory reaction will synergize with the adaptive immune response generated against the neoantigens. The following references relate to this subject matter: Daines R. A., et al., “Trisubstituted Pyridine Leukotriene B4 Receptor Antagonists: Synthesis and Structure-Activity Relationships,” J Med Chem, 36(22):3321-32 (1993); Kingsbury W. D., et al., “Synthesis of Structural Analogs of Leukotriene B4 and their Receptor Binding Activity,” J Med Chem, 36(22):3308-20 (1993), the contents of which are incorporated herein by reference in their entirety.

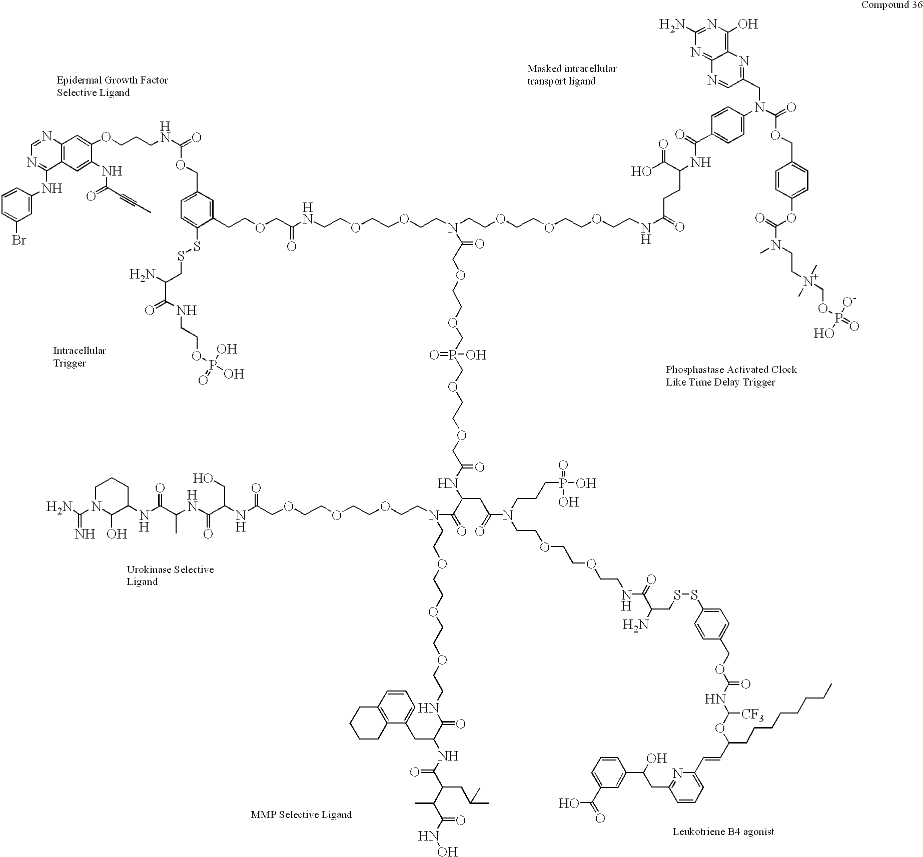

Compound 36 may be prepared using the method described for compound 34 by replacing compound 36.1 for compound 21.2.2e.

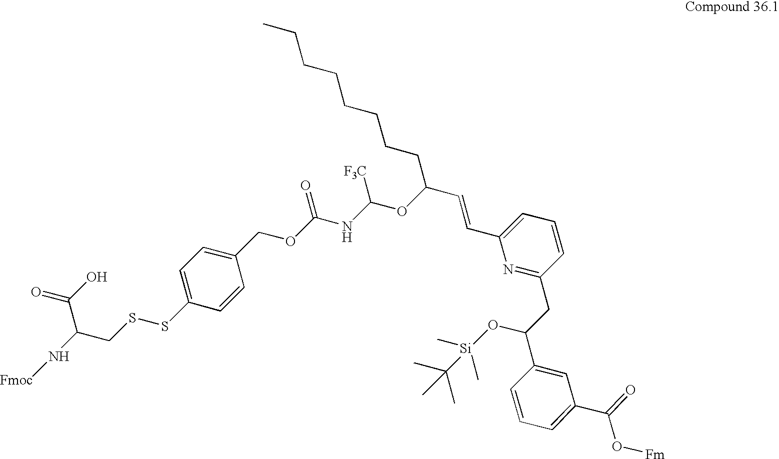

Compound 36.1 may be prepared by reacting compound 36.2 and compound 36.3 in an inert solvent in the presence of a base.

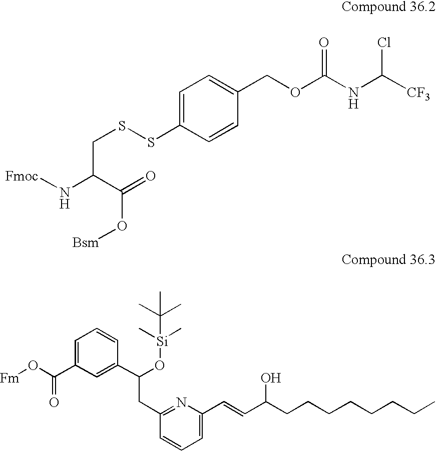

Compound 36.2 may be prepared by a multi-step process. Reacting 4-(tert-Butyl-dimethyl-silanyloxymethyl)-benzenethiol with diethylazodicarboxylate in an inert solvent and then reacting the adduct with Fmoc-L-cysteine will form the mixed disulfide compound 36.4.

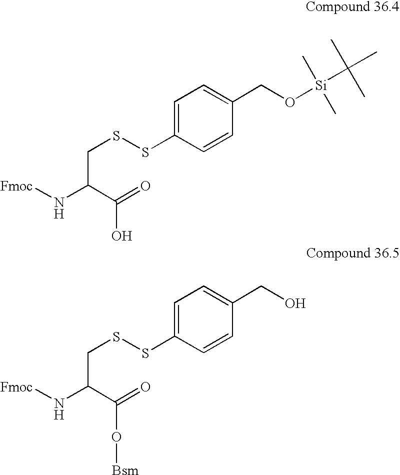

Treating with dicylcohyxylcarbodiimide and (1,1-Dioxo-1H-1λ6-benzo[b]thiophen-2-yl)-methanol in an inert solvent will give the Bsm ester. Treatment with acid will cleave the t-butyldimethyl silyl group and give compound 36.5. Treatment with phosgene in an inert solvent will give the chloroformate. Treatment with ammonia, at low temperature in an inert solvent, will give compound 36.6.

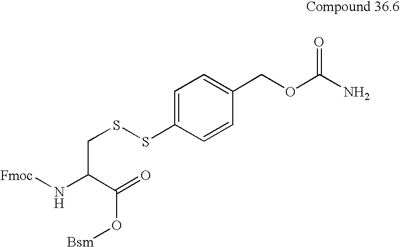

Treatment of compound 36.6 with trifluoro-acetaldehyde in an inert solvent, followed by treatment with a reagent such as phosphorous trichloride will give compound 36.2. The following references relate to this subject matter: Weygand F., et al., “2,2,2-Trifluoro-1-acylaminoethyl Groups as Protective Groups for Imino Groups of Histidine in Peptide Synthesis,” Chem Ber, 100(12):3841-9 (1967); Weygand, Friedrich; Steglich, Wolfgang; Pietta, Pier G., Chem Ber, 99: p.1944 (1966), the contents of which are incorporated herein by reference in their entirety.

Compound 36.3 may by prepared by treating compound 36.7 with trityl chloride and base in an inert solvent and then reacting with (9H-Fluoren-9-yl)-methanol and dicyclohexylcarbodiimide in an inert solvent, followed by acid treatment to remove the trityl group.

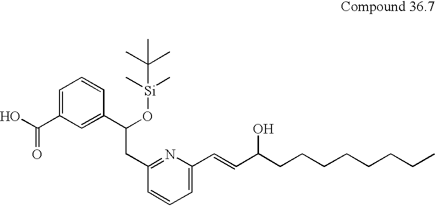

The following references relate to this subject matter: Kingsbury W. D., et al., “Synthesis of Structural Analogs of Leukotriene B4 and their Receptor Binding Activity,” J Med Chem, 36(22):3308-20 (1993), the contents of which are incorporated herein by reference in their entirety.

### Example 37

Compound 37 is a multifunctional drug delivery vehicle with targeting ligands for urokinase, matrix metalloproteinases (1, 2, 3, 9, and MT-MMP-1) and cathepsin B. Like compound 29, this compound may be used for the method of targeted neoantigen generation where the neoantigens are derived from cathepsin B.

Compound 37 also has two masked formyl butyl pyrophosphate analogs. 3-formyl-1-butyl-pyrophosphate and related derivatives are extremely potent activators of γ/δ T cells. The formyl groups will be unmasked by the action of esterase. The pyrosphosphate analog will be unmasked by an esterase activated clock like trigger that will have a half life of about 90 minutes. Synergy between the innate and adaptive immune response is expected to augment the antitumor immune response. The following references relate to this subject matter: Belmant C, et al., “3-Formyl-1-butyl Pyrophosphate a Novel Mycobacterial Metabolite-Activating Human Gammadelta T Cells,” J Biol Chem, 274(45):32079-84 (1999), the contents of which are incorporated herein by reference in their entirety.

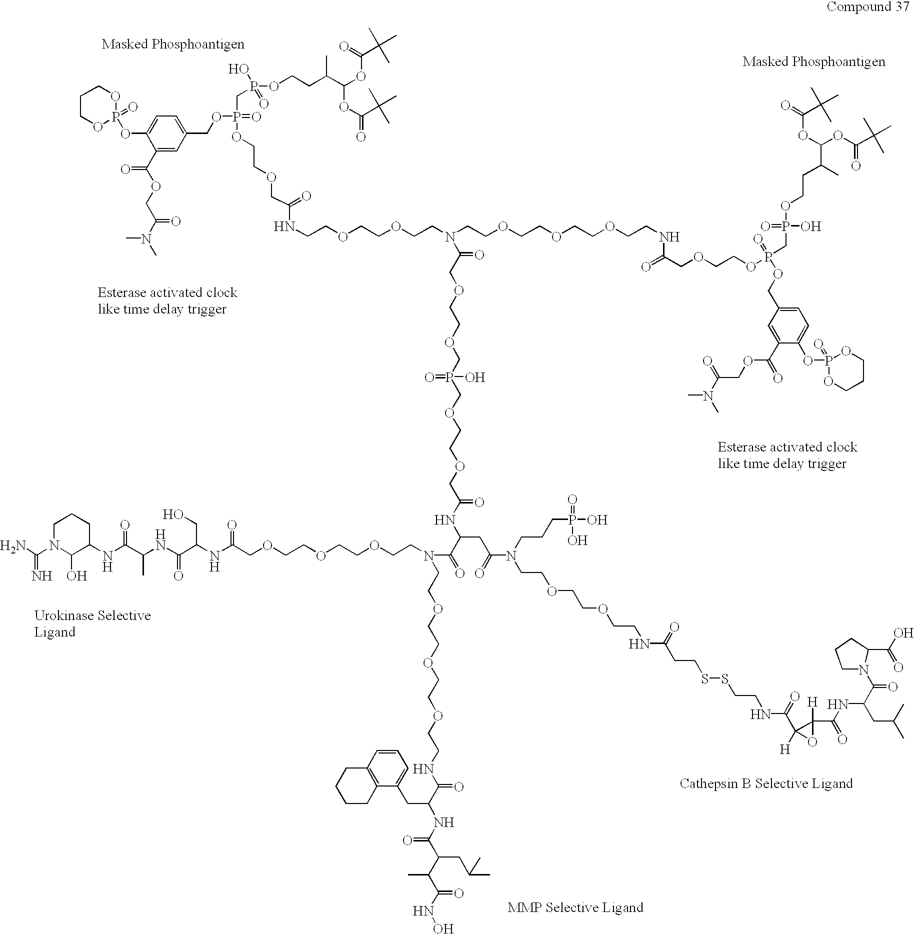

Compound 37 may be prepared by the method described for compound 29 by replacing compounds 21.1.2 and 6.2.0b with compound 37.1.

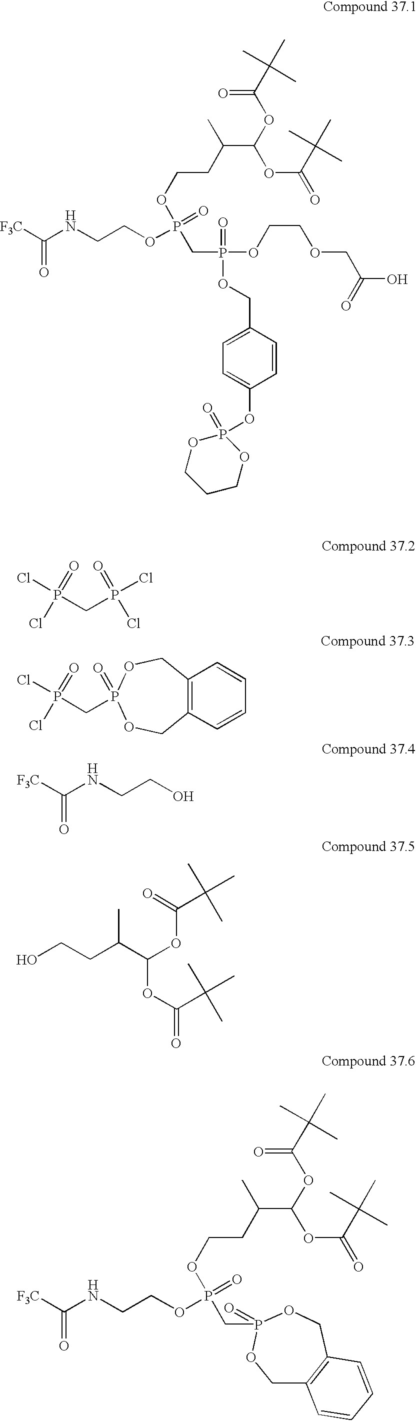

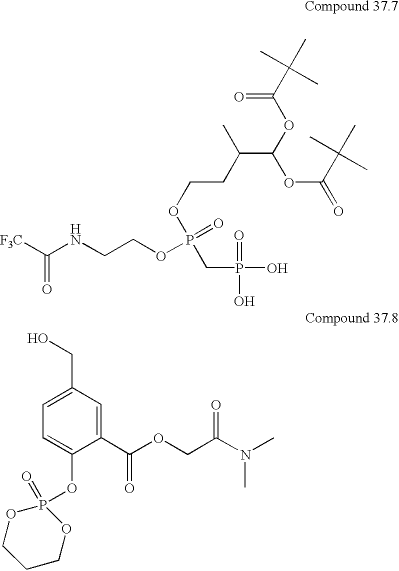

Compound 37.1 may be prepared by a multi-step process. Compound 37.2 may be reacted with (2-Hydroxymethyl-phenyl)-methanol and base in an inert solvent to give compound 37.3. Compound 37.3 may then be reacted with one equivalent of 37.4 followed by an excess of 37.5 in the presence of base, in an inert solvent to give compound 37.6 after purification by chromatography. Hydrogenation with palladium on carbon will give compound 37.7. Compound 37.7 may then be reacted with one equivalent of compound 37.8 in an inert solvent with an agent such as triisopropylbenzenesulfonyl 3-nitro-1,2,4 triazole and base in an inert solvent. Reaction of the product in a similar fashion with (2-Hydroxy-ethoxy)-acetic acid allyl ester, followed by purification by chromatography, and removal of the allyl protecting group with Pd(0) will give compound 37.1.

Compound 37.5 may be prepared by treating acetic acid 3-methyl-4-oxo-butyl ester with pivalic acid anhydride and boron trifluoride etherate in an inert solvent and then hydrolyzing the acetate ester with aqueous sodium hydroxide.

### Example 38

Compound 38 is a multifunctional drug delivery vehicle with targeting ligands for urokinase, matrix metalloproteinases (1, 2, 3, 9, and MT-MMP-1) and sigma receptors. The drug has a phosphatase activated time delay clock like trigger that will unmask the intracellular transport ligand. The drug has an intracellular trigger that will be activated by thioreductases and will free an analog of wortmannin, which is an irreversible inhibitor of phosphatidylinositol 3-kinase. The covalent modification of phosphatidylinositol 3-kinase will generate neoantigens to which the patient may be sensitized so as to elicit a targeted antitumor immunity. The following references relate to this subject matter: Creemer L. C., et al., “Synthesis and in Vitro Evaluation of New Wortmannin Esters: Potent Inhibitors of Phosphatidylinositol 3-Kinase,” J Med Chem, 39:5021-5024 (1996); Wymann M. P., et al “Wortmannin Inactivates Phosphoinositide 3-Kinase by Covalent Modification of Lys-802, A Residue Involved in the Phosphate Transfer Reaction,” Mol Cell Biol, 4:1722-33 (1996), the contents of which are incorporated herein by reference in their entirety.

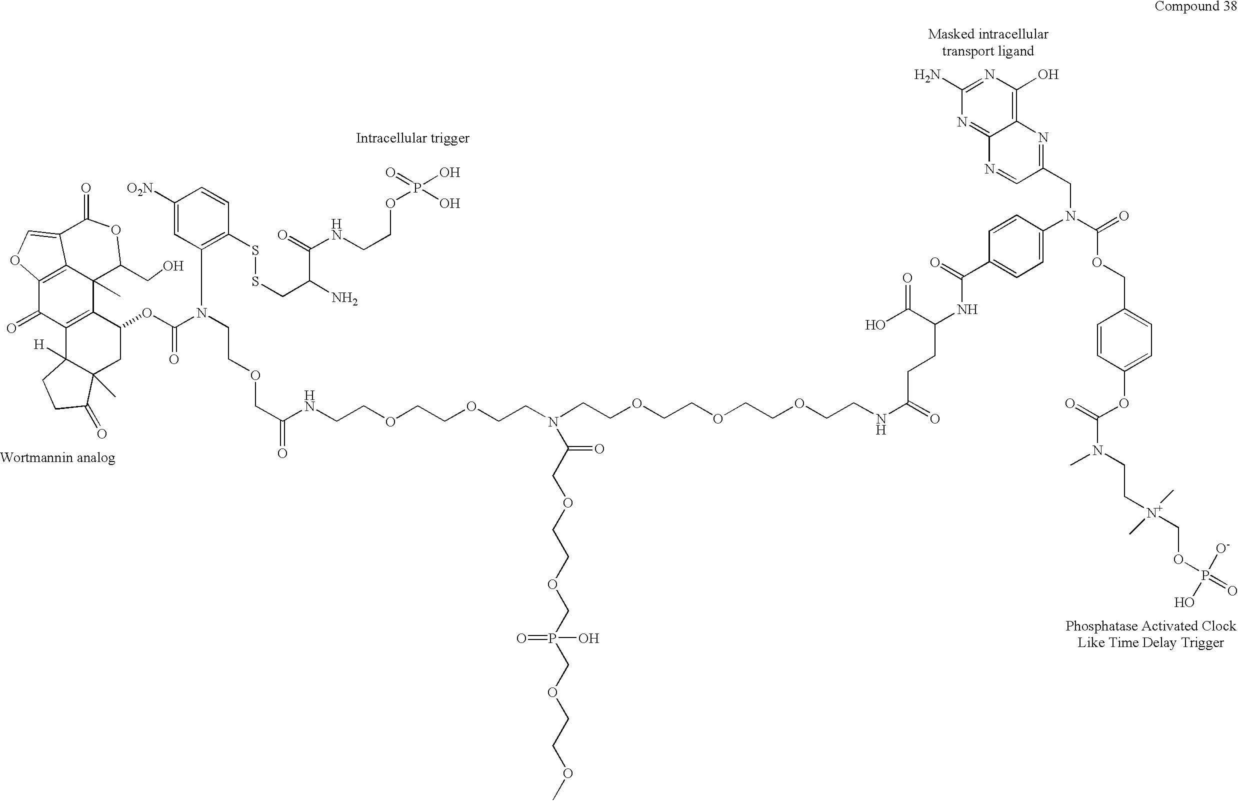

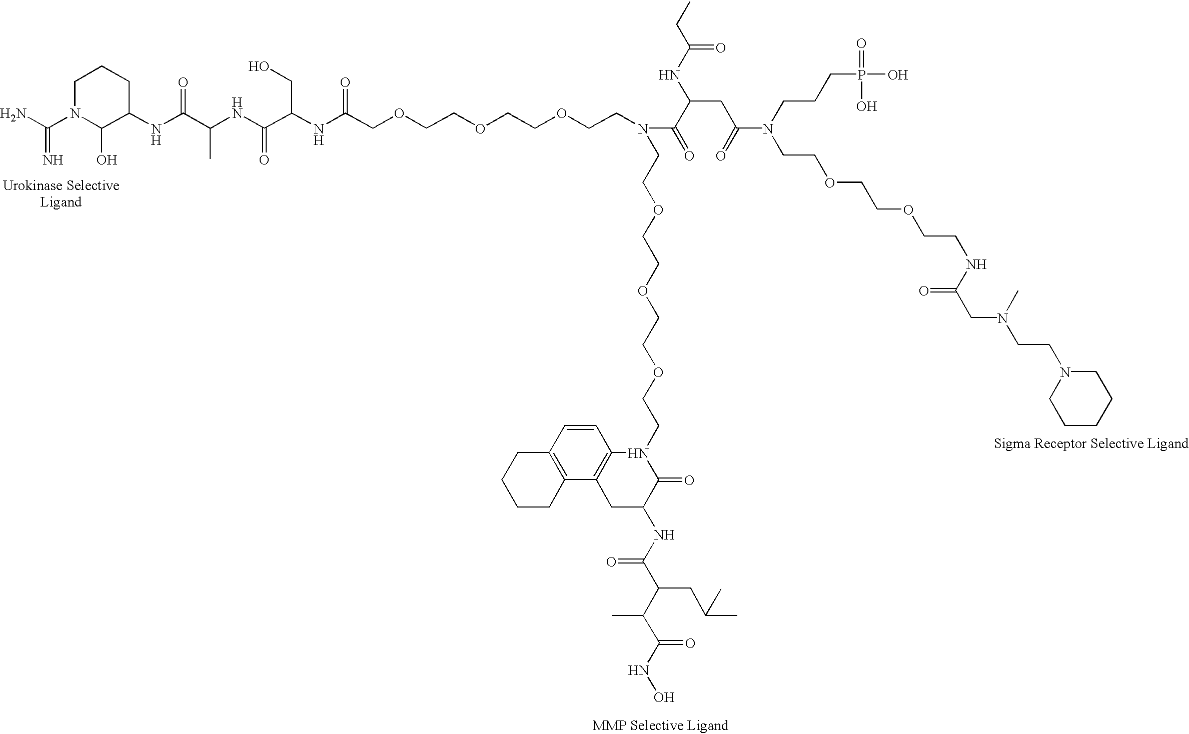

Compound 38 may be prepared by the methods described for compound 34 by replacing compound 34.1 with compound 38.1.

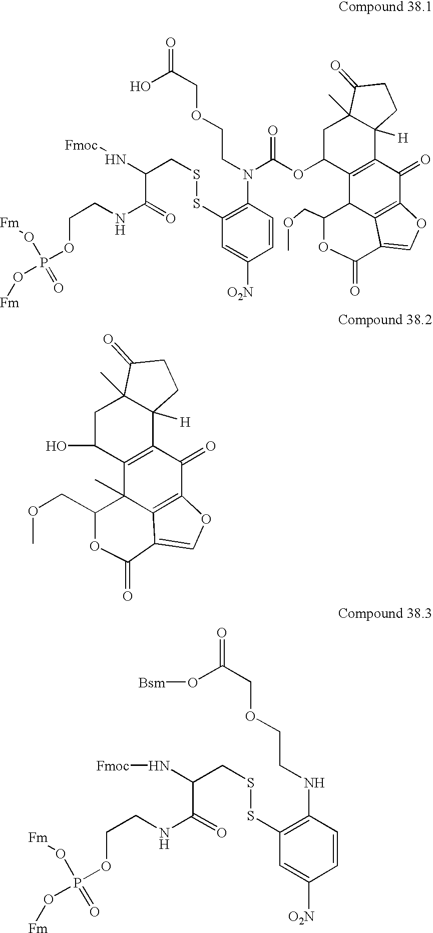

Compound 38.1 may be prepared by treating compound 38.2 with phosgene in an inert solvent, then reacting the chloroformate with compound 38.3, and then selectively removing the Bsm ester with tris(2-aminoethyl)amine under conditions that will leave the Fmoc groups intact. Compound 38.2 is a known compound. The following references relate to this subject matter: Creemer L. C., et al., “Synthesis and in Vitro Evaluation of New Wortmannin Esters: Potent Inhibitors of Phosphatidylinositol 3-Kinase,” J Med Chem, 39:5021-5024 (1996), the contents of which are incorporated herein by reference in their entirety.

Compound 38.3 may be prepared by the method described for the synthesis of compound 21.1.2b by replacing compound 17.11.1a with compound 38.4 in the synthetic scheme.

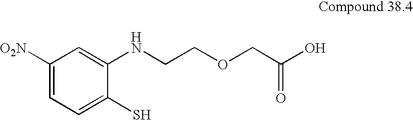

Compound 38.4 may be prepared by a multi-step procedure. Alkylation of 2-Fluoro-5-nitro-phenylamine with (2-Chloro-ethoxy)-acetic acid methyl ester in an inert solvent in the presence of base will give [2-(2-Fluoro-5-nitro-phenylamino)-ethoxy]-acetic acid methyl ester. Treatment with sodium sulfide, followed by hydrolysis of the methyl ester will give compound 38.4.

The neoantigens for sensitization may be obtained by reacting phosphoinositide 3-kinase with compound 38.2 or by employing synthetic oligopeptides with amino sequences that correspond to the modified site of the enzyme that have the wortmannin analog covalently attached in the appropriate manner.

### Example 39

Compound 39 is similar to compound 38, however the drug bears a tamoxifen analog that is linked to a masked alyklating group. The tamoxifen analog will be released and converted into an active alkylating agent upon activation of an intracellular trigger by thioreductases. The estrogen receptor will be alkylated at cysteine 530 and in the process neoantigens will be generated. P-glycoprotein will also be selectively alkylated. Accordingly, neoantigens derived from both the estrogen receptor and p-glycoprotein will be generated. Neoantigens for senstization may be prepared by treating estrogen receptor and p-glycoprotein with compound 39.2. Alternatively, synthetic oligopeptides that correspond to the modified portions of the respective proteins may be employed. The following references relate to this subject matter: Katzenellenbogen J. A., et al., “Efficient and Highly Selective Covalent Labeling of the Estrogen Receptor with [³H]Tamoxifen Aziridine,” J Biol Chem, 258(6):3487-3495 (1983); Harlow K. W., et al., “Identification of Cysteine 530 as the Covalent Attachment Site of an Affinity-labeling Estrogen (Ketononestrol Aziridine) and Antiestrogen (Tamoxifen Aziridine) in the Human Estrogen Receptor,” J Biol Chem, 264(29):17476-17485 (1989); Reese J. C.; Katzenellenbogen B. S., “Mutagenesis of Cysteines in the Hormone Binding Domain of the Human Estrogen Receptor,” 266(17):10880-10887 (1991); Aliau S., et al., “Cysteine 530 of the Human Estrogen Receptor α is the Main Covalent Attachment Site of 11β-(Aziridinylalkoxyphenyl)estradiols,” Biochemistry, 38:14752-14762 (1999); Robertson D. W., et al., “Tamoxifen Aziridines: Effective Inactivators of the Estrogen Receptor,” Endocrinology, 109(4):1298-300 (1981); Safa A. R., et al., “Tamoxifen Aziridine, a Novel Affinity Probe for P-glycoprotein in Multidrug Resistant Cells,” Biochem Biophys Res Commun, 202(1):606-12 (1994), the contents of which are incorporated herein by reference in their entirety.

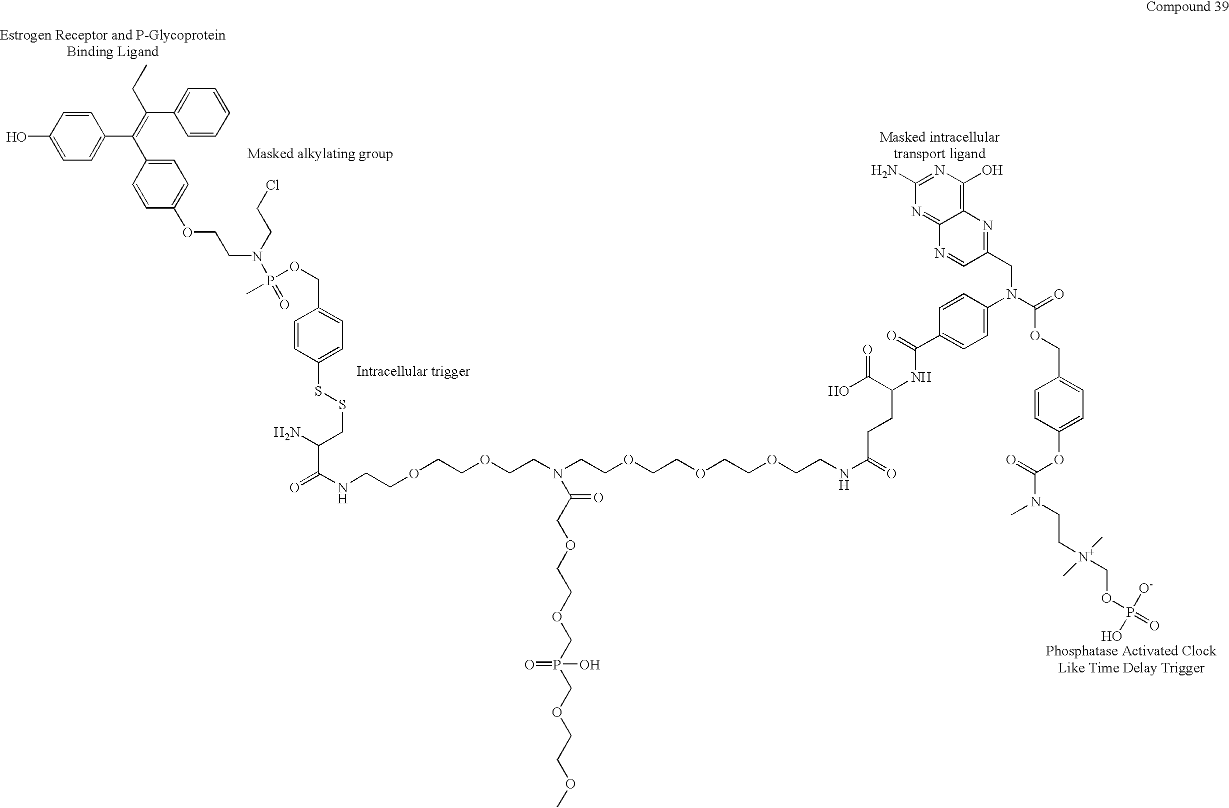

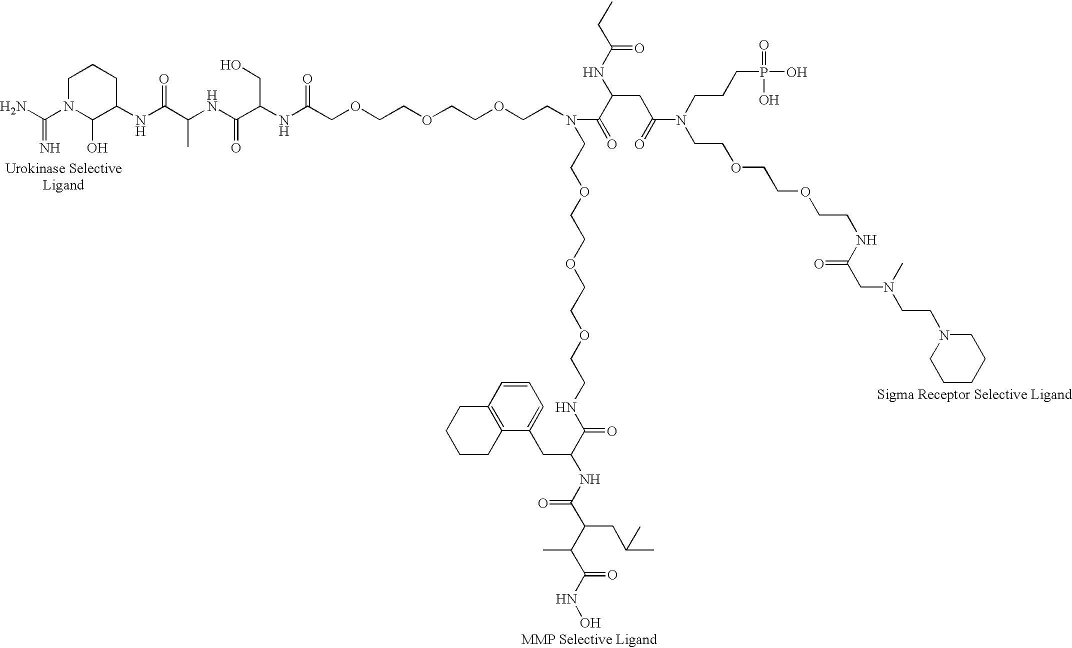

Compound 39 may be prepared by the methods described for compound 38 by replacing compound 38.1 with compound 39.1.

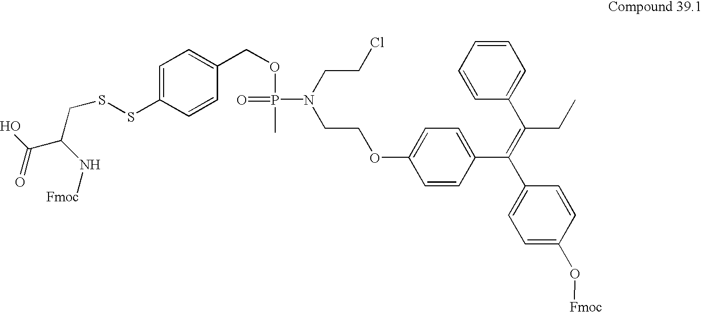

Compound 39.1 may be prepared by reacting compound 36.5 and compound 39.2 in an inert solvent in the presence of base and then selectively cleaving the Bsm ester with tris(2-aminoethyl)amine under conditions that will leave the Fmoc groups intact.

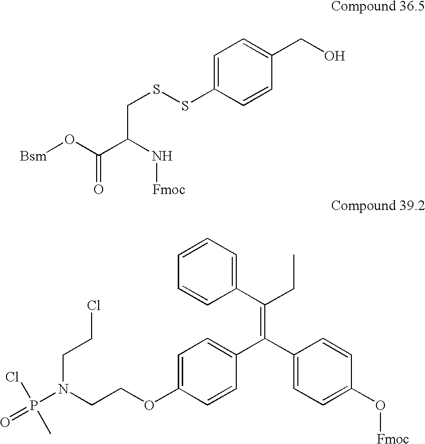

Compound 39.2 may be prepared by treating compound 39.3 with methylphosphonic acid dichloride and base in an inert solvent. Compound 39.3 may be prepared by treating compound 39.4 with HCl or thionyl chloride in an inert solvent.

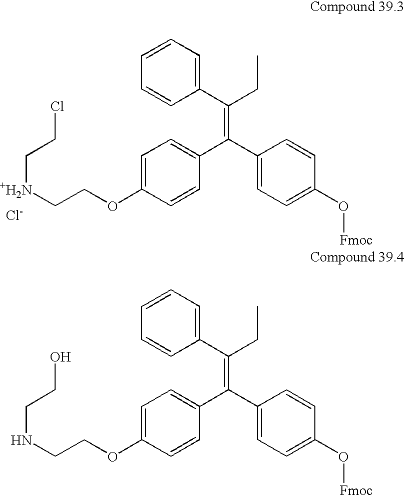

Compound 39.4 may be made by a multi-step process. Treatment of 2-[2-(4-Bromo-phenoxy)-ethoxy]-tetrahydro-pyran with n-butyl lithium in an inert solvent followed by reaction with 2-Phenyl-1-[4-(tetrahydro-pyran-2-yloxy)-phenyl]-butan-1-one and HCl treatment will give compound 39.5. The following references relate to this subject matter: Katzenellenbogen J. A., et al., “Efficient and Highly Selective Covalent Labeling of the Estrogen Receptor with [³H]Tamoxifen Aziridine,” J Biol Chem, 258(6):3487-3495 (1983), the contents of which are incorporated herein by reference in their entirety.

Treatment with sodium hydride and pivaloyl chloride in an inert solvent will give compound 39.6.

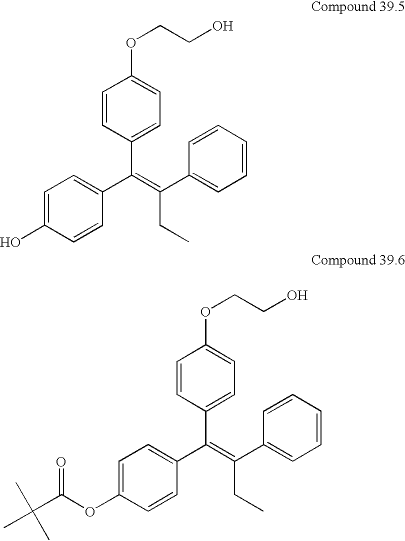

Treatment with tosyl chloride and base in an inert solvent will give the tosylate that may then be reacted with ethanolamine to give compound 39.7. Treatment with di-t-butyl pyrocarbonate and in an inert solvent with a base will give compound 39.8.

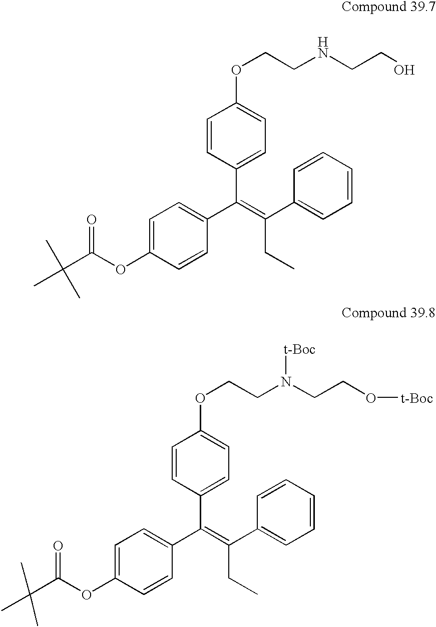

Alkaline hydrolysis of compound 39.8 will selectively cleave the pivaloyl ester and give compound 39.9a. Treatment with 9-fluorenylmethyl chloroformate, in an inert solvent with base, will then give compound 39.9b. Removing the t-Boc groups with acid will give compound 39.4.

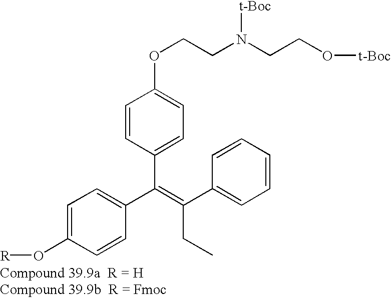

### Example 40

Compound 40 is similar to compound 39, however, the tamoxifen analog has a phosphate group to increase solubility. The phosphate group will be cleaved by esterases to generate a ligand with binding affinity to the estrogen receptor.

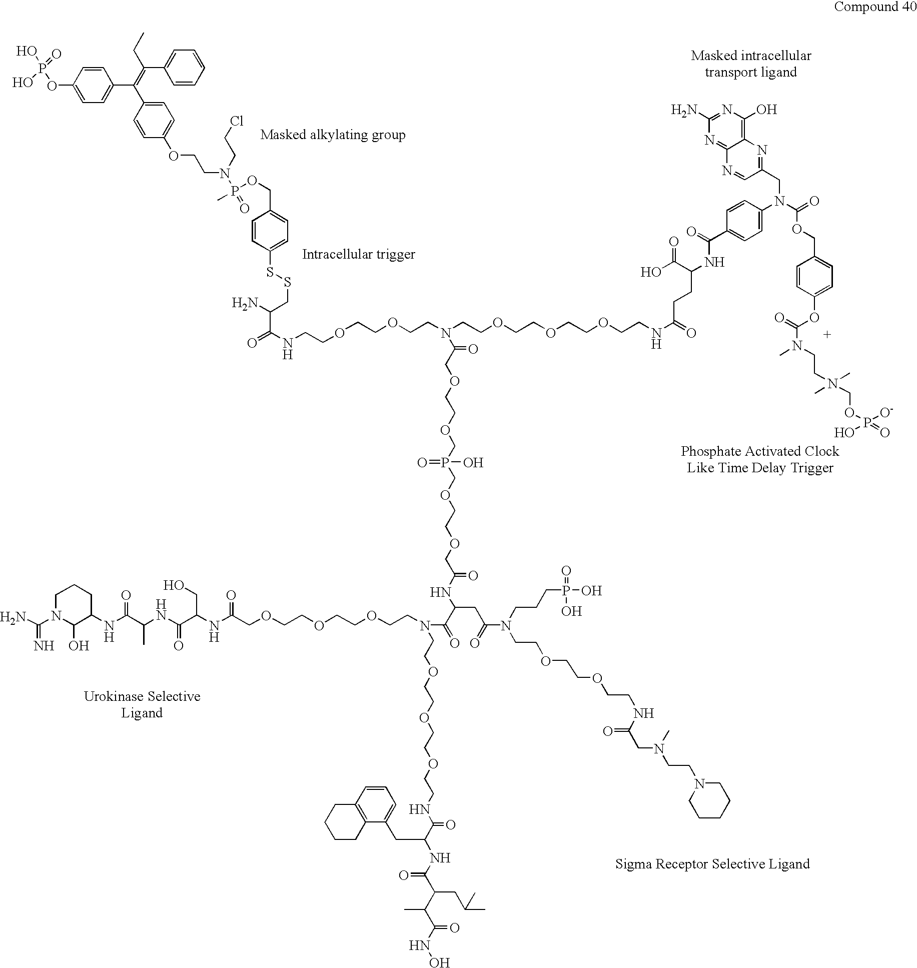

Compound 40 may be prepared by the method described for compound 39 by reacting compound 39.9a with phosphorochloridic acid bis-(9H-fluoren-9-ylmethyl) ester and a base such as triethylamine in an inert solvent (in the place of Fmoc chloroformate).

### Example 41

Compound 41 is similar to compound 38, however, the drug will deliver cerulenin, an irreversible inhibitor of fatty acid synthase. The interaction of cerulenin and fatty acid synthase will generate neoantigens in a targeted manner. The following references relate to this subject matter: Funabashi H., et al., “Binding Site of Cerulenin in Fatty Acid Synthetase,” J Biochem, 105:751-755 (1989); Moche M., et al., “Structure of the Complex between the Antibiotic Cerulenin and Its Target, β-Ketoacyl-Acyl Carrier Protein Synthase,” J Biological Chem, 274(10):6031-6034 (1999), the contents of which are incorporated herein by reference in their entirety.

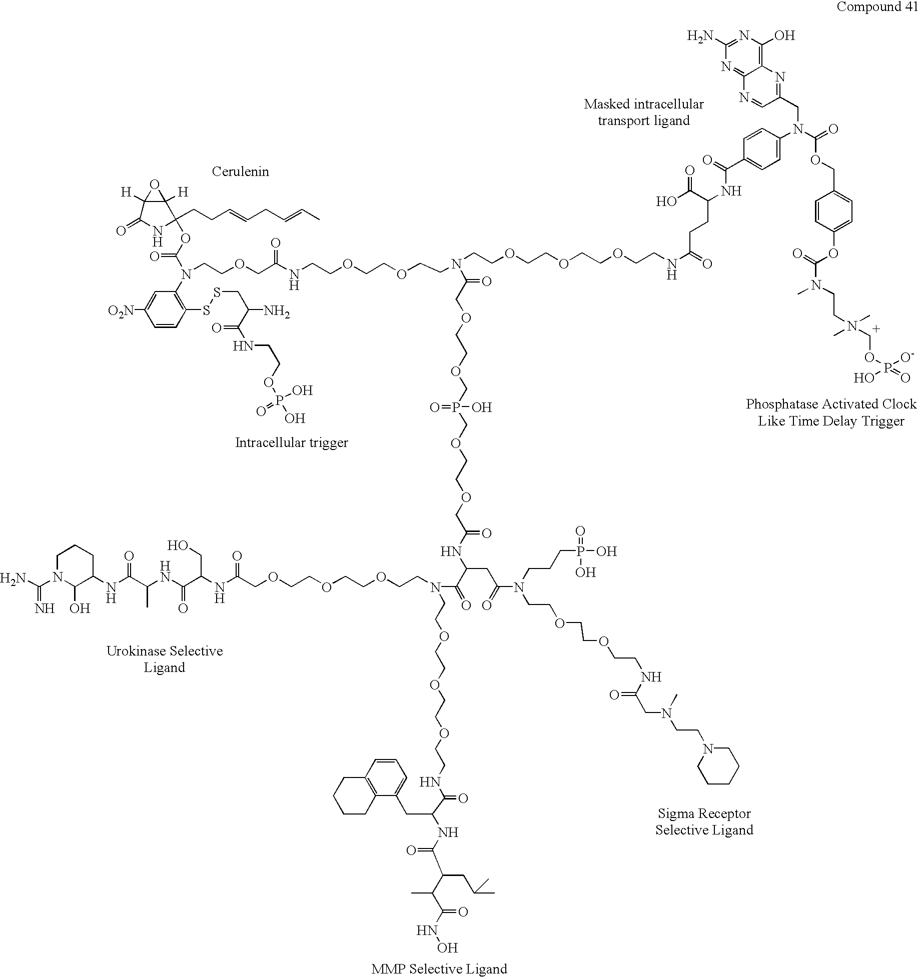

Compound 41 may be prepared by the method described for compound 38 by replacing compound 41.1 for compound 38.1.

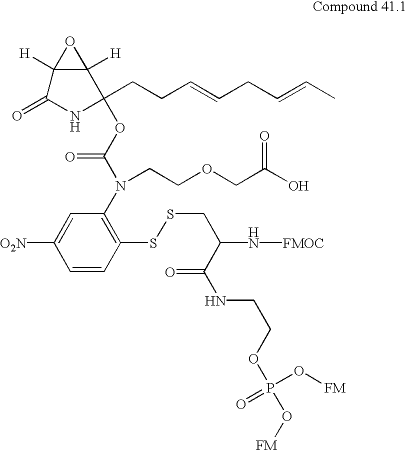

Compound 41.1 may be prepared by reacting compound 38.3 with phosgene in an inert solvent, then reacting the product with cerulenin in the presence of a base, and then selectively removing the Bsm ester with tris(2-aminoethyl)amine under conditions that will leave the Fmoc groups intact.

### Example 42

Compound 42 is similar to compound 41, however, a different trigger is used to release the cerulenin. The trigger will be activated by thioreductases, which will free the n-methyl-phosphate derivative of cerulenin, which will be degraded by phosphatase to cerulenin.

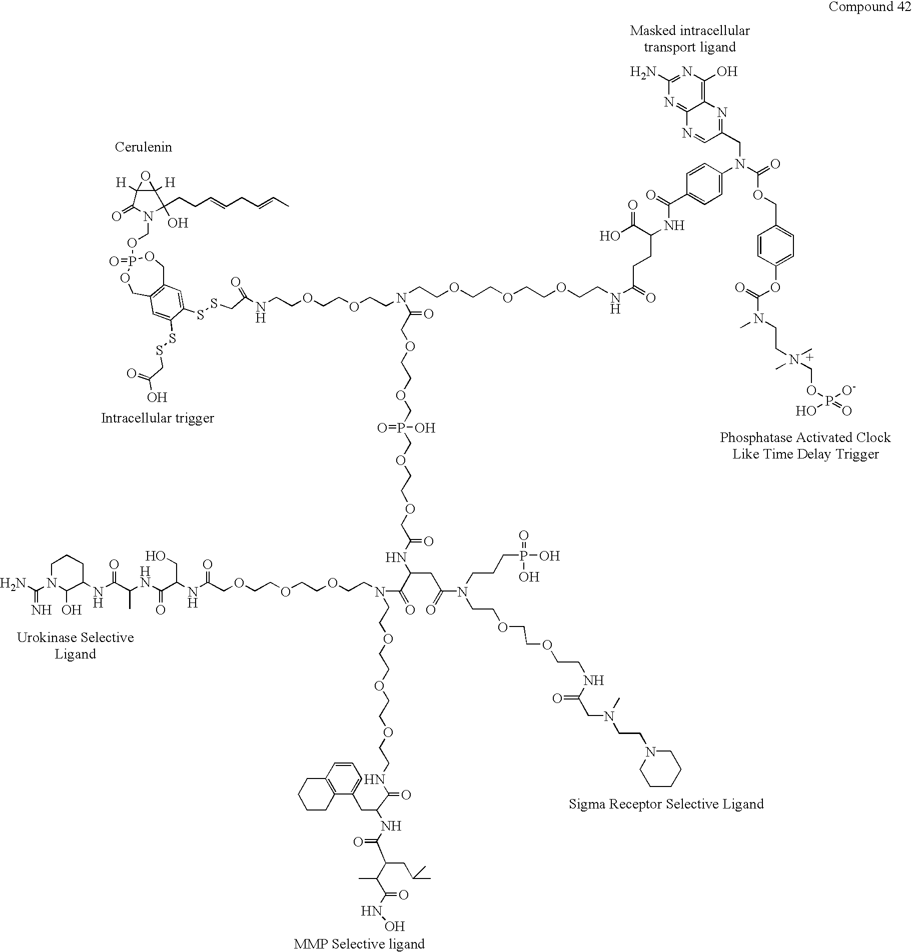

Compound 42 may be prepared by the method described for compound 38 by replacing compound 38.1 with compound 42.1.

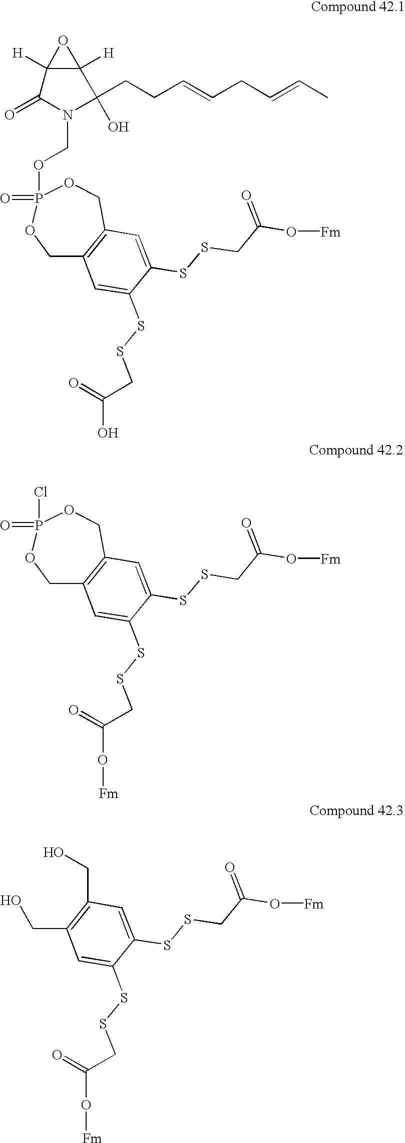

Compound 42.1 may be prepared by a multi-step process. Reacting cerulenin with formaldehyde in an inert solvent, then reacting the n-hydroxymethylated product with compound 42.2 in the presence of base, and then treating with one equivalent of strong base, will give compound 42.1 after purification by chromatography. The following references relate to this subject matter: Bundgaard H., “Formation of Prodrugs of Amines, Amides, Ureides, and lmides,” Methods in Enzymology, 112:347-359 (1985), the contents of which are incorporated herein by reference in their entirety.

Compound 42.2 may be prepared by reacting compound 42.3 with phosphorous oxychloride in an inert solvent in the presence of base. Compound 42.3 may be prepared by reacting mercapto-acetic acid 9H-fluoren-9-ylmethyl ester with diethyl azidocarboxylate in an inert solvent and then reacting the product with compound 42.4.

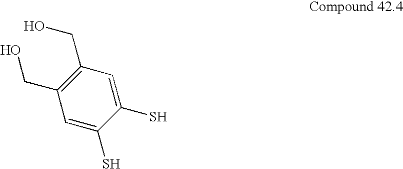

Compound 42.4 may be prepared by a multi-step process. Reacting 4,5-Dichloro-phthalic acid with sodium ethanethiolate in dimethylformamaide will give 4,5-dimercapto-phthalic acid. Reduction with borane in a solvent such as tetrahydrofuran will give compound 42.4. The following references relate to this subject matter: Testaferri L., et al., “Simple Syntheses of Aryl Alkyl Thioethers and of Aromatic Thiols from Unactivated Aryl Halides and Efficient Methods for Selective Dealkylation of Aryl Alkyl Ethers and Thioethers,” Synthesis, 751-755 (1983)., the contents of which are incorporated herein by reference in their entirety.

Neoantigens for sensitization may be prepared by treating fatty acid synthase with cerulenin. Alternatively, synthetic oligopeptides that correspond to the modified portions of the fatty acid synthase may be employed.

### Example 43

Compound 43 is a multifunctional drug delivery vehicle with targeting ligands for urokinase, MMPs 2, 3, 9,12, and 13, and sigma receptors. The drug has a masked intracellular transporter with a phosphatase activated time delay clock like trigger and will release resorcylic acid lactone upon activation of an intracellular trigger by thioreductase. Resorcylic acid lactone is a potent irreversible inhibitor of MEK. The interaction of resorcylic acid lactone and MEK will generate neoantigens that may be used in the method of targeted immunotherapy. The following references relate to this subject matter: Zhao A., et al., “Resorcylic Acid Lactones: Naturally Occurring Potent and Selective Inhibitors of MEK,” J Antibiotics, 52(12):1086-1094 (1999); Hoshino R., et al., “Constitutive Activation of the 41-/43-kDa Mitogen-activated Protein Kinase Signaling Pathway in Human Tumors,” Oncogene, 18:813-822 (1999), the contents of which are incorporated herein by reference in their entirety.

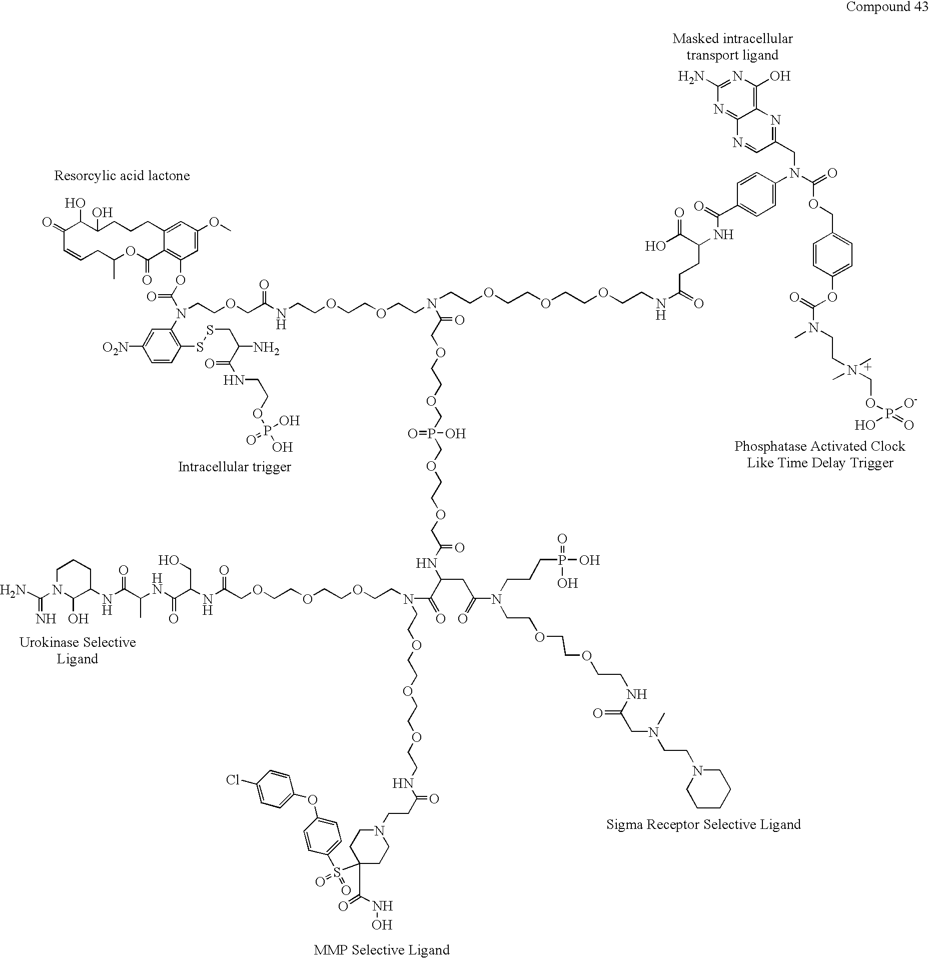

Compound 43 may be prepared by the methods described for compound 38 by replacing compound 38.2 with compound 43.1a or compound 43.1b and also by replacing compound 21.2.1d with compound 18.1.

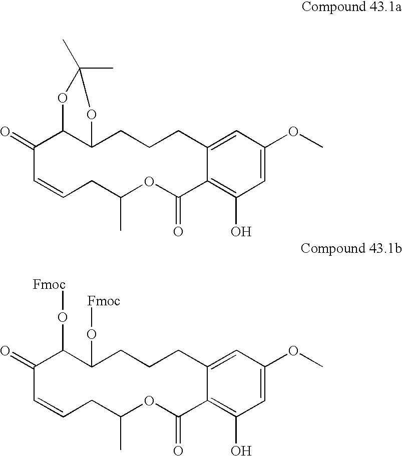

Compound 43.1a may be prepared by routine methods of acetonide formation from resorcylic acid lactone. The following references relate to this subject matter: Greene, Theodora W.; Wuts, Peter G. M. (1999) “Protective Groups in Organic Synthesis” John Wiley & Sons, Inc. p 207-213, the contents of which are incorporated herein by reference in their entirety.

Compound 43.1b may be prepared by treating compound 43.1a with (1,1-Dioxo-H-1λ6-benzo[b]thiophen-2-yl)-methyl chloroformate and base in an inert solvent, then treating with acid to cleave the acetonide protecting group, then treating with 9H-fluoren-9-ylmethyl chloroformate in the presence of a base, and then selectively removing the Bsmoc group with tris(2-aminoethyl)amine under conditions that will leave the Fmoc group intact.

The neoantigens for sensitization purposes may be prepared by treating MEK with resorcylic acid lactone. Alternatively, synthetic oligopeptides that correspond to the modified MEK with resorcylic acid lactone covalently attached may be employed.

### Example 44

Compound 44 is a multifunctional drug delivery vehicle with a targeting ligand for prostatic specific membrane antigen, a nonspecific targeting ligand for cell membranes, and an irreversible inhibitor for prostate specific antigen. The interaction of the PSA inhibitor and PSA will generate neoantigens for use with the method of targeted neoantigen immuntherapy. The role of the nonspecific membrane binding ligand is to enhance the affinity of the drug for PSMA positive cells. The PSMA binding ligand will bind with a Ki in the low nanomolar range to PSMA. The additional binding energy provided by the membrane binding ligand to the cell should provide for essentially irreversible binding to PSMA positive cells.

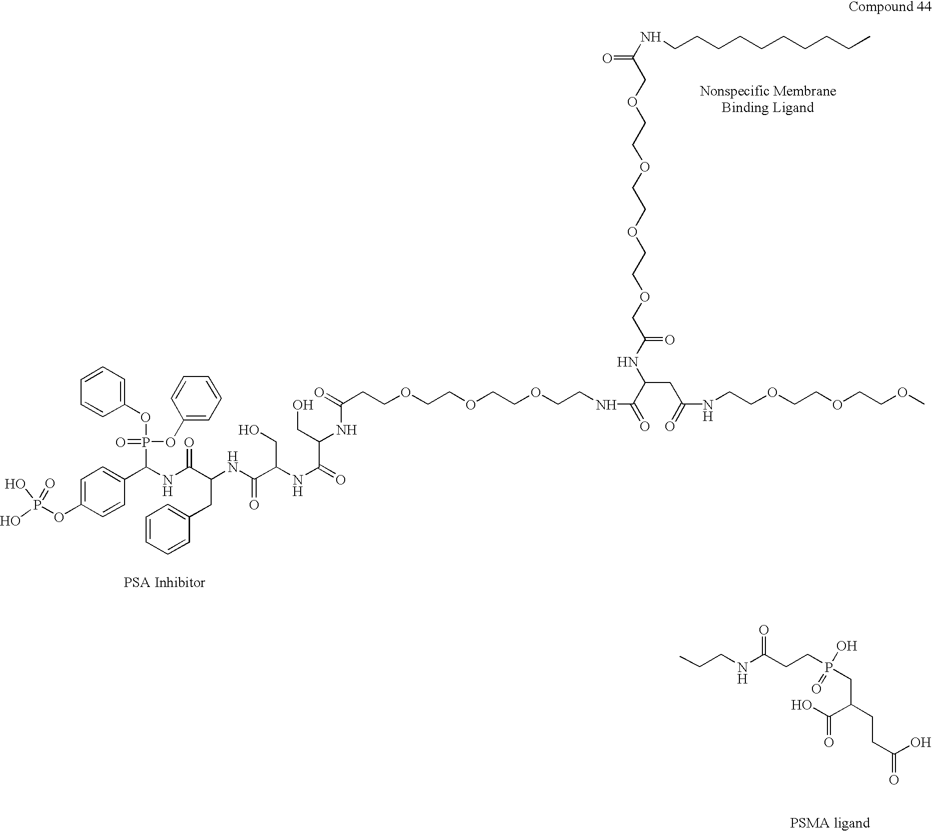

Compound 44 may be prepared by a multi-step process. Coupling L-aspartic acid α2,2,2 trichloroethyl β benzyl diester with compound 44.1 in an inert solvent will give compound 44.2.

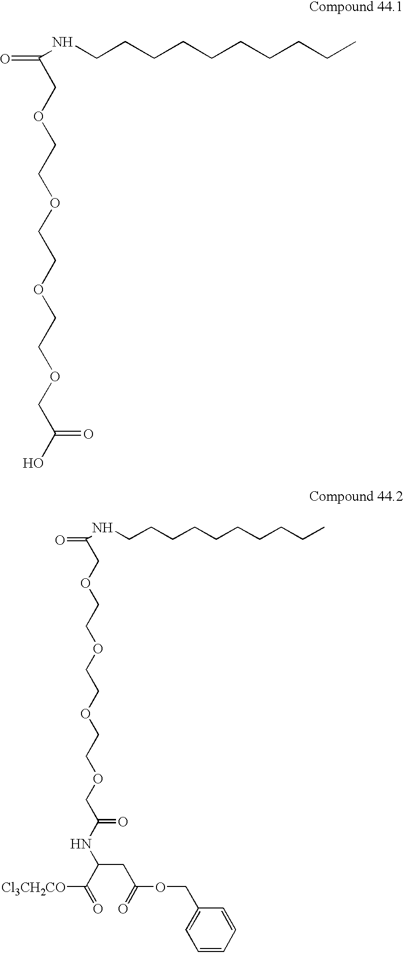

Removal of the benzyl group by catalytic hydrogenation with Pd on carbon and coupling to compound 44.3 will give compound 44.4.

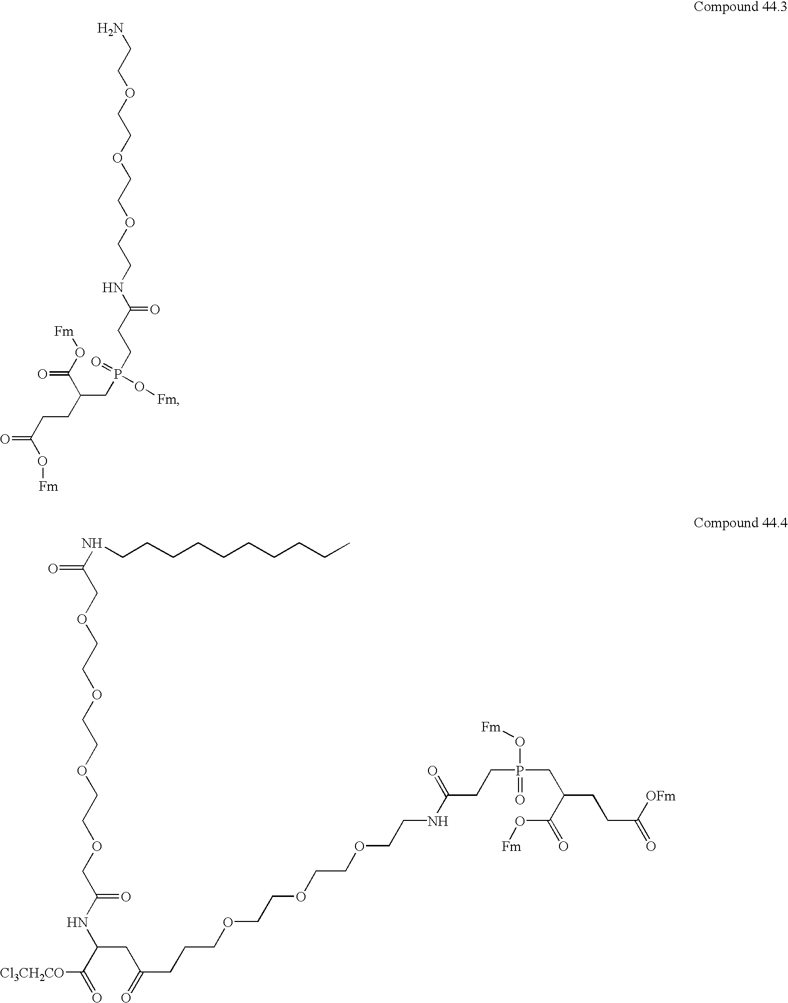

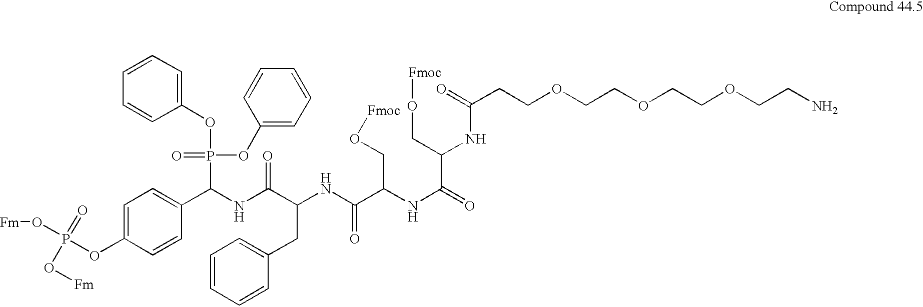

Compound 44.4 may be treated with Zn and acid to cleave the 2,2,2 trichloroethyl ester. The product may then be coupled with compound 44.5. Treatment of the product with base will remove the Fmoc and cleave the Fm ester groups and give compound 44.

Compound 44.1 may be prepared by coupling {2-[2-(2-Carboxymethoxy-ethoxy)-ethoxy]-ethoxy}-acetic acid with decylamine and isolating the monosubstituted product.

Compound 44.3 may be prepared by a multi-step process. Coupling 2-(2-{2-[2-(Trityl-amino)-ethoxy]-ethoxy}-ethoxy)-ethylamine and compound 6.6.1 followed by alkaline hydrolysis will give compound 44.3b.

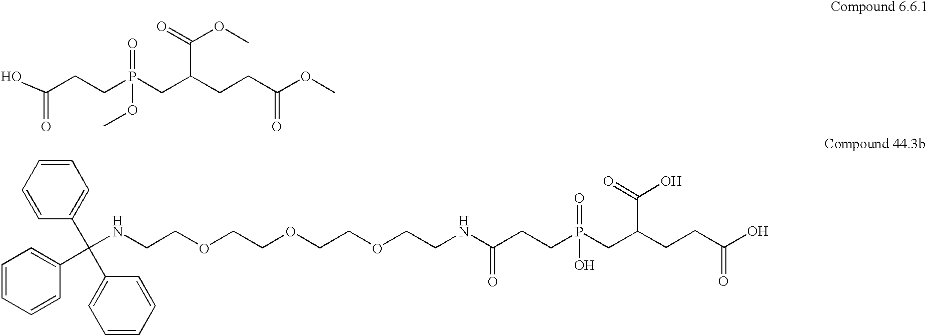

Compound 44.3b may be esterified with (9H-Fluoren-9-yl)-methanol and a condensing agent such as dicyclohexylcarbodiimide or triisopropylbenzenesulfonyl 3-nitro-1,2,4 triazole and base in an inert solvent.

Treatment with HCL will remove the trityl protecting group and give compound 44.3 as the hydrochloride salt.

Compound 44.5 may be prepared by a multi-step process.

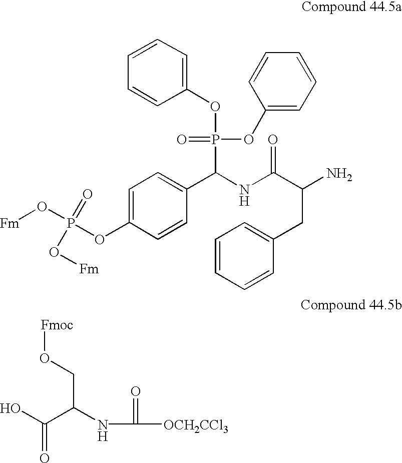

Compound 44.5a may be coupled with compound 44.5b. The product may be treated with Zn and acid to remove the 2,2,2 trichloroethyl group and the product may again be coupled with compound 44.5b. Treatment with Zn and acid followed by coupling to 3-(2-{2-[2-(2,2,2-Trichloro-ethoxycarbonylamino)-ethoxy]-ethoxy}-ethoxy)-propionic acid and removal of the trichloro-ethoxycarbonyl protecting group with Zn and acid will give compound 44.5.

Compound 44.5a may be prepared by a multi-step process.

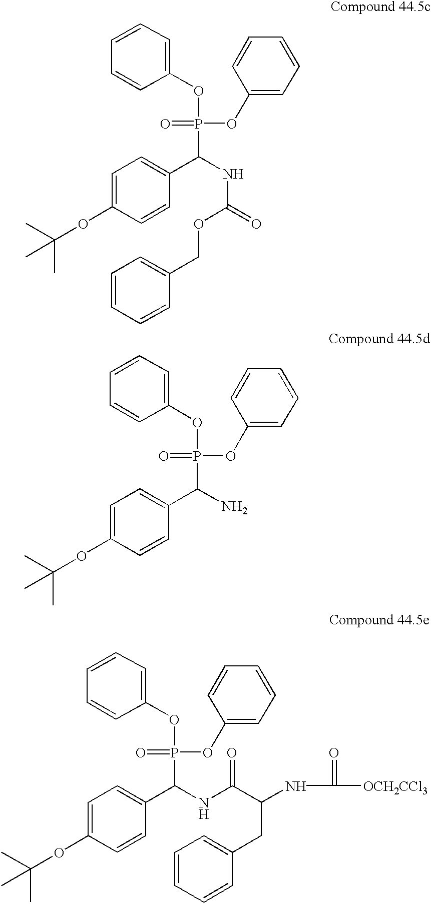

Reacting 4-tert-butoxy-benzaldehyde, benzyl carbamate, triphenyl phosphite, and glacial acetic acid will give compound 44.5c. Catalytic hydrogenation with Pd on carbon in methanol with HCL will give compound 44.5d. The following references relate to this subject matter: Oleksyszyn J., et al., “Novel Amidine-Containing Peptidyl Phosphonates as Irreversible Inhibitors for Blood Coagulation and Related Serine Proteases,” J Med Chem, 37:226-231 (1994); Oleksyszyn J., et al., “Diphenyl 1-Aminoalkanephosphonates,” Synthesis, 985-986 (1979), the contents of which are incorporated herein by reference in their entirety.

Coupling to L-N-2,2,2 trichloroethoxylcarbonyl phenylalanine will give compound 44.5e. Treatment with trifluoracetic acid will remove the t-butyl group. Treatment of the product with phosphorochloridic acid bis-(9H-fluoren-9-ylmethyl) ester and a base such as triethylamine in an inert solvent, followed by removal of the 2,2,2 trichloroethylcarbonyl protecting group with Zn and acid will give compound 44.5a.

The neoantigen for sensitization purposes may be prepared by treating PSA with compound 44.6. Alternatively, synthetic oligopeptides that correspond to the modified amino acid sequence of PSA with the inhibitor covalently attached may be employed.

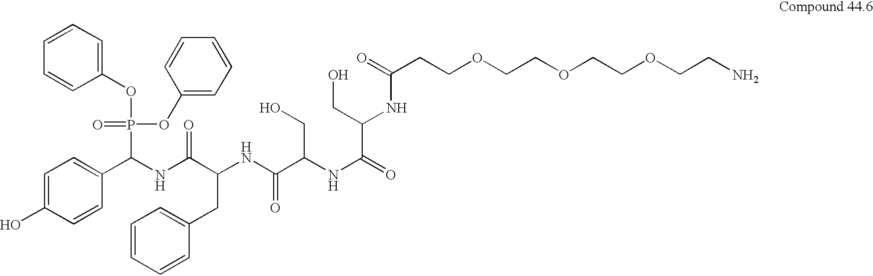

### Example 45

Compound 45 is similar to compound 44. However, in compound 45a haolenol lactone based inhibitor is used to covalently modify PSA and generate neoantigens.

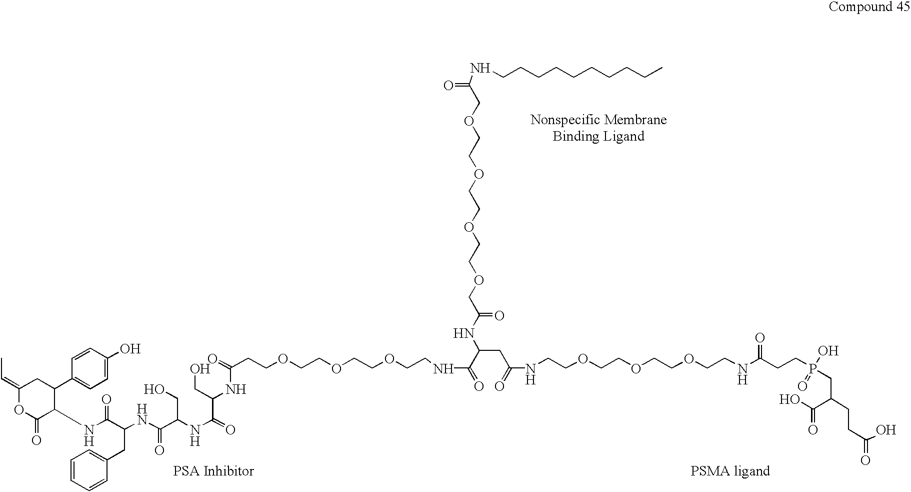

Compound 45 may be prepared by the method described for compound 44 by replacing compound 44.5 with compound 45.1.

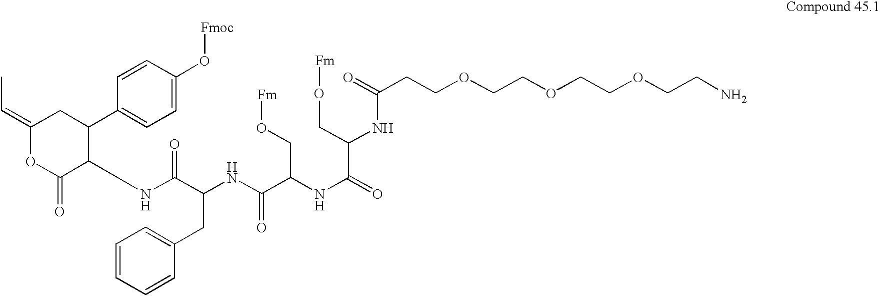

Compound 45.1 may be prepared by a multi-step procedure. Reacting ethyl nitroacetate and compound 45.2 in an inert solvent in the presence of a base such as sodium hydride will give compound 45.3. The following references relate to this subject matter: Sofia M. J.; Katzenellenbogen J. A., “Enol Lactone Inhibitors of Serine Proteases. The Effect of Regiochemistry on the Inactivation Behavior of Phenyl-Substituted (Halomethylene)tetra- and -dihydrofuranones and (Halomethylene)tetrahydropyranones toward α-Chymotrypsin: Stable Acyl Enzyme Intermediate,” J Med Chem, 29:230-238 (1986); Läuger P., et al., “Carbinols, Carbamates et esters Propynyliques, et Leur Activite Hypnotique,” Helv Chim Acta, 42:2379-2393 (1959), the contents of which are incorporated herein by reference in their entirety.

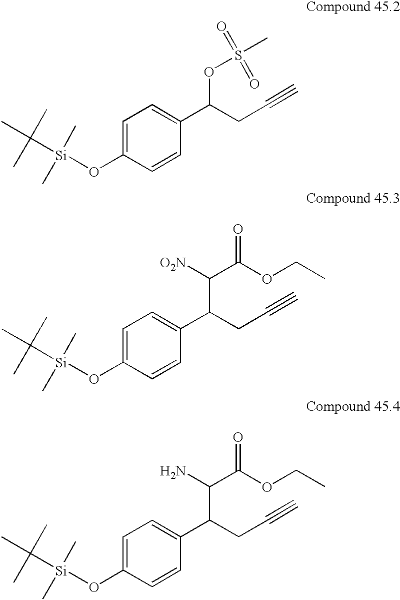

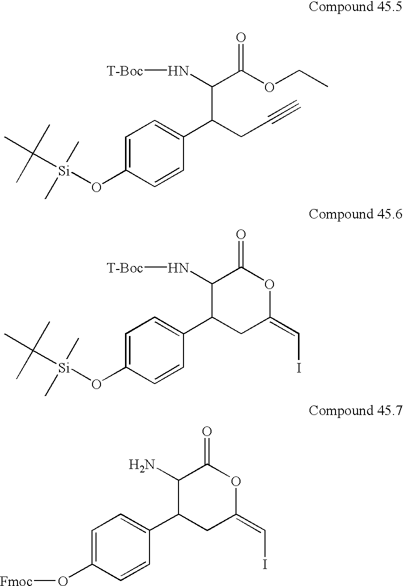

Selective reduction of the nitro group by a reagent such as diethylchlorophosphite will give compound 45.4. The following references relate to this subject matter: Fischer B.; Sheihet L., “Diethyl Chlorophosphite: A Mild Reagent for Efficient Reduction of Nitro Compounds to Amines,” J Org Chem, 63:393-395 (1998), the contents of which are incorporated herein by reference in their entirety.

Treatment with di-t-butyl pyrocarbonate and in an inert solvent will give compound 45.5.

In an alternate method, the free acid derivative of compound 45.5 may be prepared by reacting compound 45.2 and 2-tert-Butoxycarbonylamino-malonic acid dimethyl ester in the presence of a strong base, the hydrolyzing the methyl esters, and heating to decarboxylate. The following references relate to this subject matter: Rai R. ; Katzenellenbogen J. A., “Effect of Conformational Mobility and Hydrogen-Bonding Interactions on the Selectivity of Some Guanidinoaryl-Substituted Mechanism-Based Inhibitors of Trypsin-like Serine Proteases,” J Med Chem, 35:4297-4305 (1992), the contents of which are incorporated herein by reference in their entirety.

Hydrolysis of the methyl ester of compound 45.5 followed by treatment with iodine in an inert solvent will give compound 45.6. Treatment with tetrabutylammonium fluoride or acid will remove the t-butyidimethylsilyl protecting group. Treatment with 9-fluorenylmethyl chloroformate and base in an inert solvent followed by acid treatment will give compound 45.7.

Compound 45.7 may be coupled with t-Boc L-phenylalanine. The product may be treated with acid to remove the t-Boc group and then may be coupled with N-t-Boc, O-Fmoc-L-serine (L-2-tert-Butoxycarbonylamino-3-(9H-fluoren-9-ylmethoxycarbonyloxy)-propionic acid). The t-Boc group may again be removed and the product may then be coupled to another N-Boc-O-Fmoc-L-serine. Removal of the t-Boc group, followed by coupling to 3-{2-[2-(2-tert-butoxy-carbonylamino-ethoxy)-ethoxy]-ethoxy}-propionic acid, followed by removal of the t-Boc group will give compound 45.1.

The neoantigen for sensitization purposes may be prepared by treating PSA with a compound such as compound 45.8. Alternatively, synthetic oligopeptides that correspond to the modified amino acid sequence of PSA with the inhibitor covalently attached may be employed.

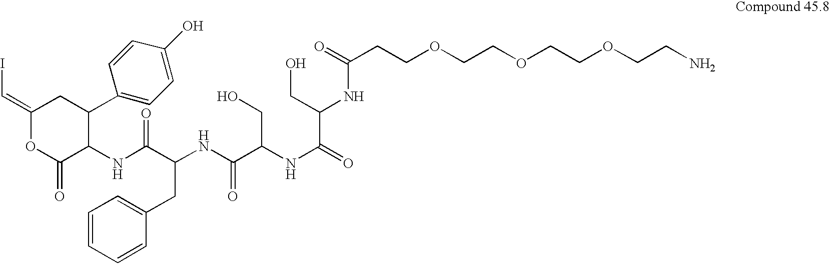

### Example 46

Compound 46 has targeting ligands for MMPs 2, 3, 9, 12, and 13, Fibroblast Activation Protein (FAP), and Seprase. The drug will bind irreversibly to FAP and seprase and generate neoantigens that may be used for targeted immunotherapy.

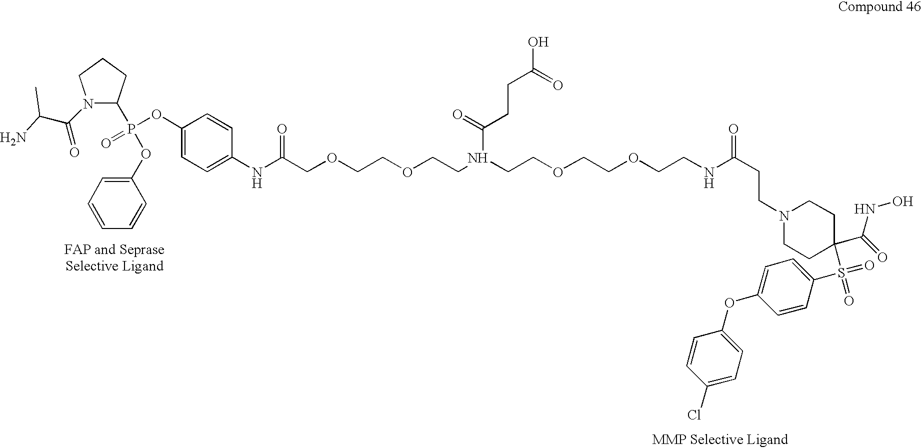

Compound 46 may be prepared by a multistep process. Coupling compound 17.7 with succinic acid mono-(9H-fluoren-9-ylmethyl) ester will give compound 46.1.

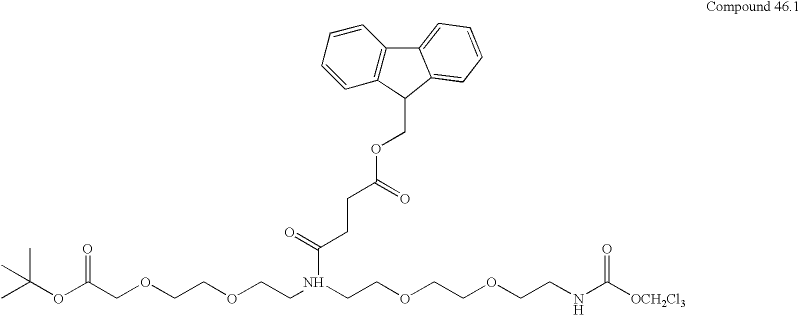

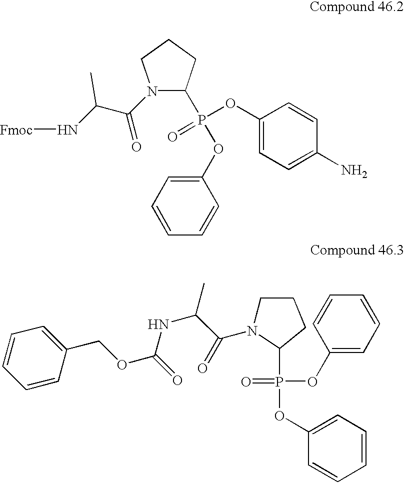

Treatment with trifluoroacetic acid will cleave the t-butyl ester group. The product may then be coupled to compound 46.2. Treatment with Zn and acid will cleave the 2,2,2 trichloroethoxycarbonyl group. The product may then be coupled to compound 18.1. Treatment with base will remove the protecting groups and give compound 46.

Compound 46.2 may be prepared by a multi-step process. Treating compound 46.3 with sodium hydroxide will give compound 46.4. The following references relate to this subject matter: Belyaev A., et al., “Structure-Activity Relationship of Diaryl Phosphonate Esters as Potent Irreversible Dipeptidyl Peptidase IV Inhibitors,” J Med Chem, 42:1041-1052 (1999); Belyaev A., et al., “A New Synthetic Method for Proline Diphenyl Phosphonates,” Tetrahedron Let, 36(21):3755-3758 (1995), the contents of which are incorporated herein by reference in their entirety.

Esterification with (4-hydroxy-phenyl)-carbamic acid tert-butyl ester using a reagent such as as triisopropyl-benzenesulfonyl 3-nitro-1,2,4 triazole and base in an inert solvent will give compound 46.5.

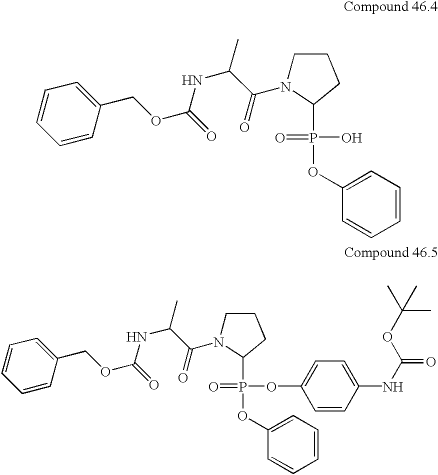

Catalytic hydrogenation with Pd on carbon, followed by treatment with 9H-fluoren-9-ylmethyl chloroformate in the presence of a base such as pyridine in an inert solvent, followed by treatment with acid to remove the t-Boc group will give compound 46.2.

The neoantigens for sensitization purposes may be prepared by treating FAP and seprase with an inhibitor such as compound 46.6 Alternatively, synthetic oligopeptides that correspond to the modified portion of FAP and seprase with the inhibitor covalently attached may be employed.

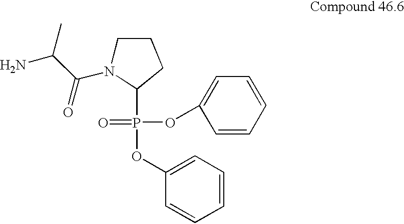

### Example 47

Compound 47 is a multifunctional drug delivery vehicle with targeting ligands for serine proteases, matrix metalloproteinases (1, 2, 3, 9, and MT-MMP-1), and cathepsin B. The compound is similar to compound 37, but has a haloenol lactone derivative that is expected to irreversibly inactivate and, in the process, generate neoantigens from a variety of trypsin like serine proteases. This compound may be used for the method of targeted neoantigen generation where the neoantigens are derived from cathepsin B, urokinase, plasmin tissue plasminogen activator, trypsin, and human glandular kallikrein 2. Compound 47 also has two masked formyl butyl pyrophosphate analogs. 3-formyl-1-butyl-pyrophosphate and related derivatives are extremely potent activators of γ/δ T cells. The formyl groups will be unmasked by the action of esterase. The pyrosphosphate analog will be unmasked by an esterase activated clock like trigger that will have a half life of about 90 minutes. Synergy between the innate and adaptive immune response is expected to augment the antitumor immune response. The following references relate to this subject matter: Rai R.; Katzenellenbogen J. A., “Guanidinophenyl-Substituted Enol Lactones as Selective, Mechanism-Based Inhibitors of Trypsin-like Serine Proteases,” J Med Chem, 35:4150-4159 (1992), the contents of which are incorporated herein by reference in their entirety. 780

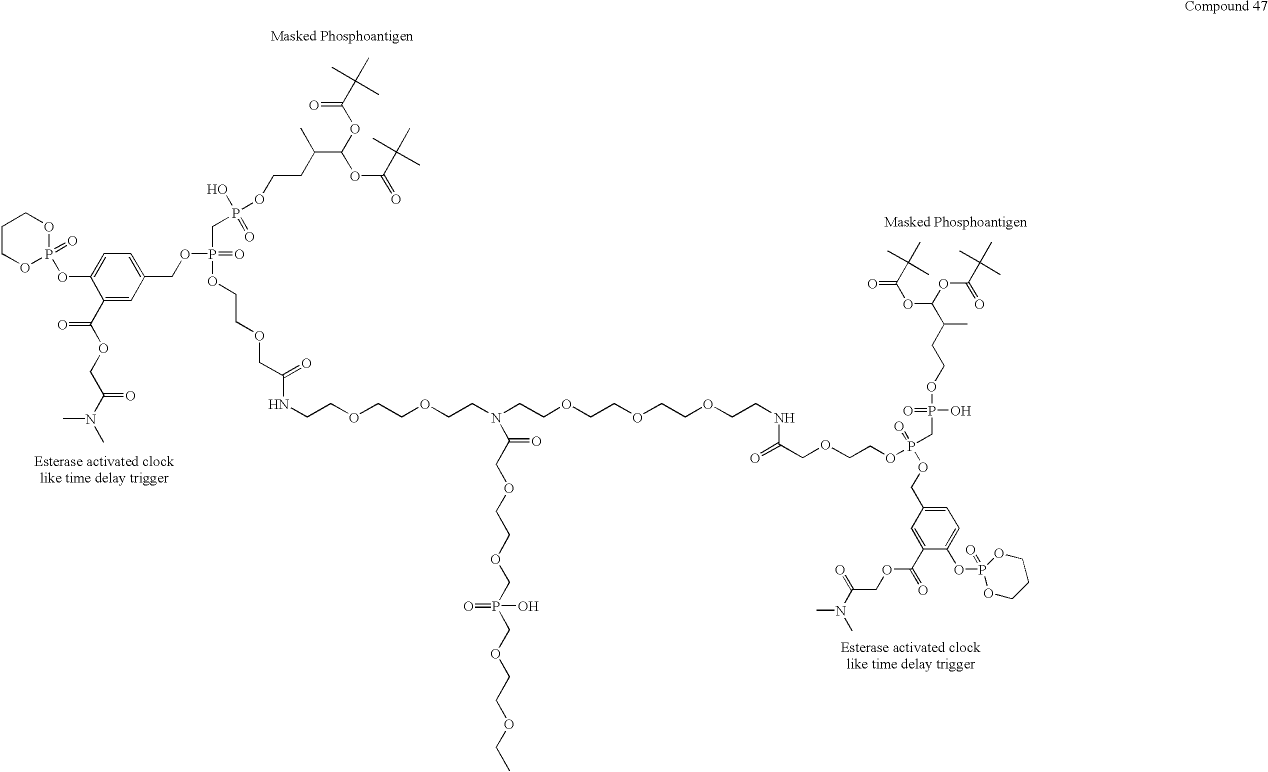

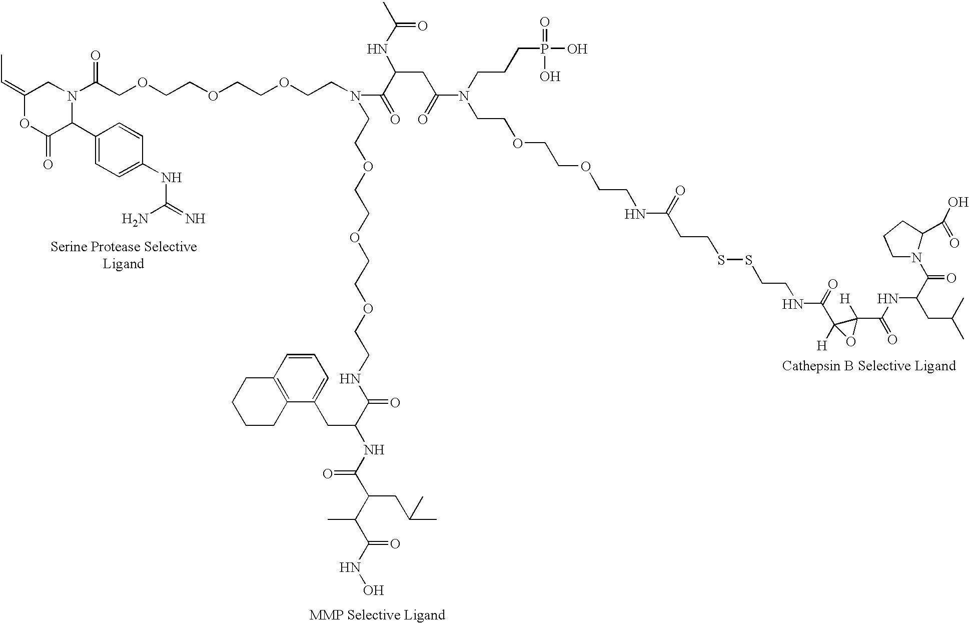

Compound 47 may be prepared by the method described for compound 37 by replacing compound 21.2.1b with compound 47.1.

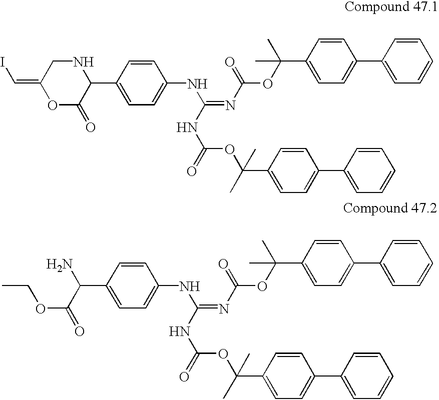

Compound 47.1 may be prepared by a multi-step process. Alkylating compound 47.2 with 3-Bromo-propyne in the presence of base in an inert solvent will give compound 47.3. Hydrolysis of the ethyl ester followed by treatment with 9H-fluoren-9-ylmethyl chloroformate in the presence of a base such as pyridine in an inert solvent will give compound 47.4.

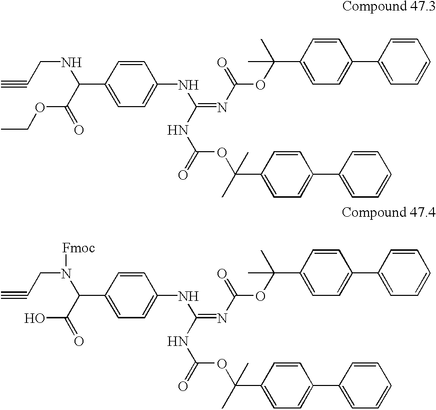

Treatment of compound 47.4 with iodine will give compound 47.5.

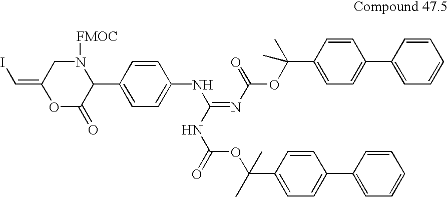

Treatment with base will give compound 47.1.

Compound 47.2 may be prepared by a multi-step process. P-amino-L-phenyl glycine ethyl ester may be treated with 9H-fluoren-9-ylmethyl chloroformate in the presence of a base to give compound 47.6. Treatment with compound 47.7 in the presence of base in an inert solvent, followed by removal of the Fmoc group with base, will give compound 47.2.

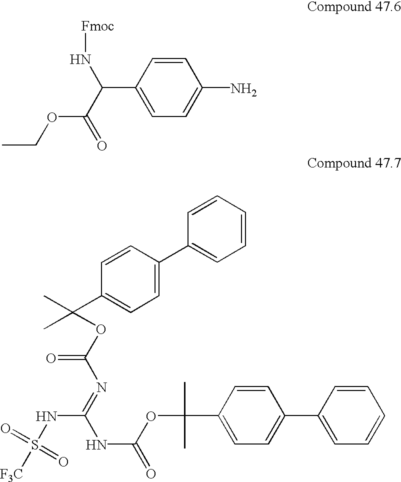

Compound 47.7 may be prepared by treating guanidine with a strong base and a reagent such as 4-(1-Biphenyl-4-yl-1-methyl-ethoxycarbonyloxy)-benzoic acid methyl ester followed by treatment with a base such as sodium hydride and triflic anhydride. The following references relate to this subject matter: Feichtinger K., et al., “Diprotected Triflylguanidines: A New Class of Guanidinylation Reagents,” J Org Chem, 63:3804-3805 (1998), the contents of which are incorporated herein by reference in their entirety.

The neoantigens for sensitization purposes may be prepared by treating the respective serine proteases with an inhibitor such as compound 47.8 (as the dihydrocloride salt). Alternatively, synthetic oligopeptides that correspond to the modified portion of the proteases with the inhibitor covalently attached may be employed.

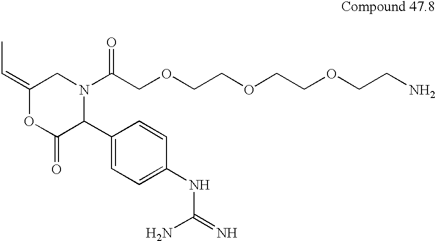

### Example 48

Compound 48 is similar to compound 47, however the enol lactone inhibitor has a para amidino group rather than a para guanidino group. Compound 48 may be prepared by replacing compound 47.2 with compound 48.1 in the method described for compound 47.

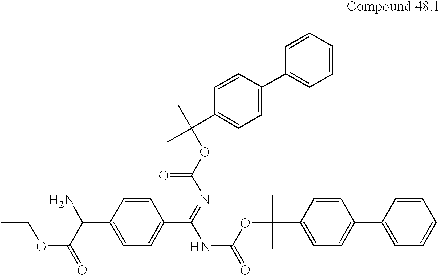

Compound 48.1 may be prepared by a multi-step process. Treating L-p-amidino phenylglycine ethyl ester with one equivalent of carbonic acid 4-nitro-phenyl ester 2-trimethylsilanyl-ethyl ester and base in an inert solvent will give after purification compound 48.2.

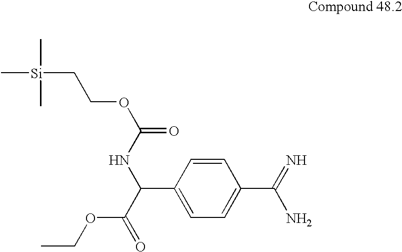

Treating compound 48.2 with base and a reagent such as 4-(1-Biphenyl-4-yl-1-methyl-ethoxycarbonyloxy)-benzoic acid methyl ester in an inert solvent and then removing the silyl based protecting group with tetra-butylammonium fluoride will give compound 48.1.

Neoantigens for sensitization purposes may be prepared by reacting the respective proteases with an inhibitor such as compound 48.3 (as the dihydrocloride salt). Alternatively, synthetic oligopeptides that correspond to the modified portion of the proteases with the inhibitor covalently attached may be employed.

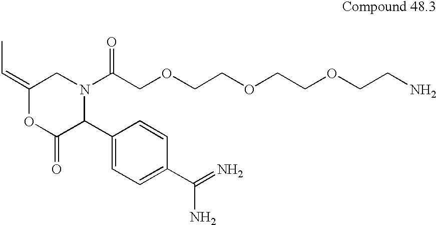

### Example 49

Compound 49 is similar to compound 47 except the masked phosphoantigen activators of γ/δ T cells have been replaced with ligands that will bind to the N-formyl peptide receptor after unmasking. Activation of the N-formyl peptide receptor will induce leukocyte chemotaxis, superoxide generation, and the release of inflammatory cytokines. These will all synergize with the immune response directed towards the neoantigens generated from the covalent modification of cathepsin B and the targeted trypsin like serine proteases. The N-formyl peptide receptor ligands are masked by esterase triggered clock like time delay tiggers. The following references relate to this subject matter: Higgins J. D., et al., “N-Terminus Urea-Substituted Chemotactic Peptides: New Potent Agonists and Antagonists toward the Neutrophil fMLF Receptor,” J Med Chem, 39(5):1013-1015 (1996), the contents of which are incorporated herein by reference in their entirety.

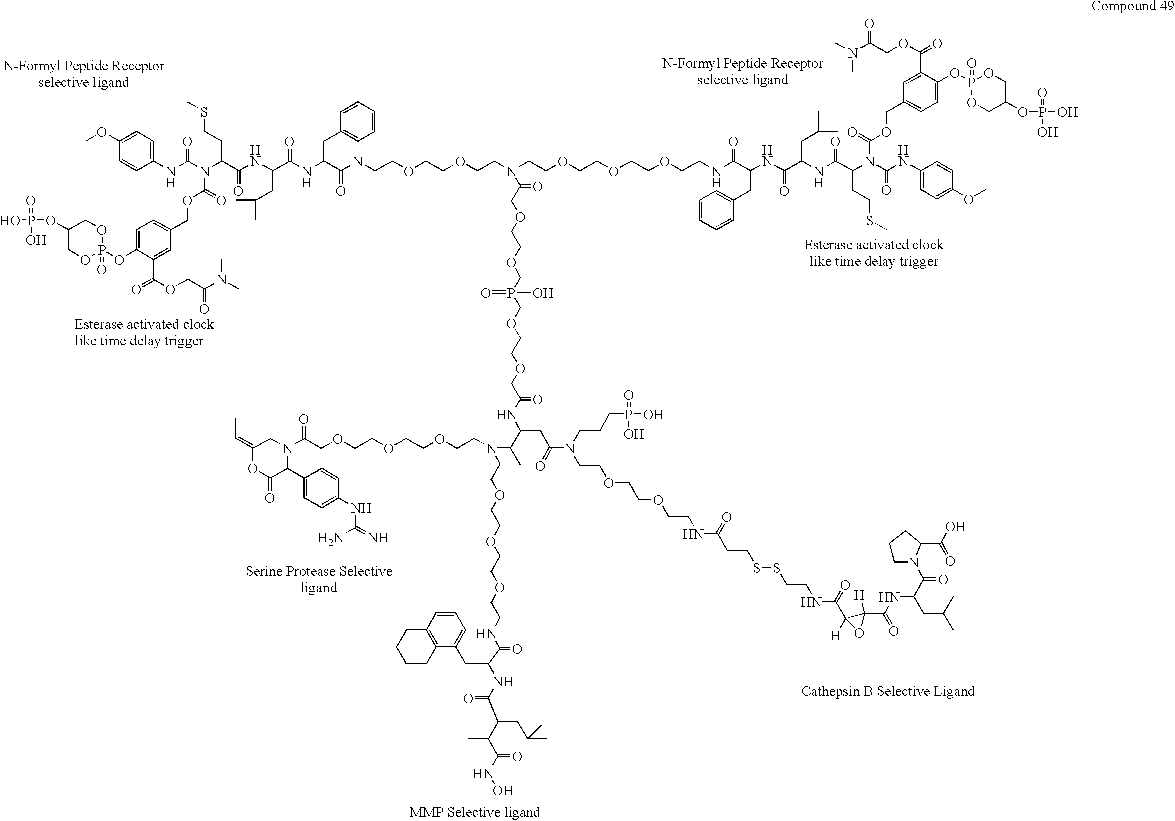

Compound 49 may be prepared by the methods described for compound 47 by replacing compound 37.1 with compound 49.1.

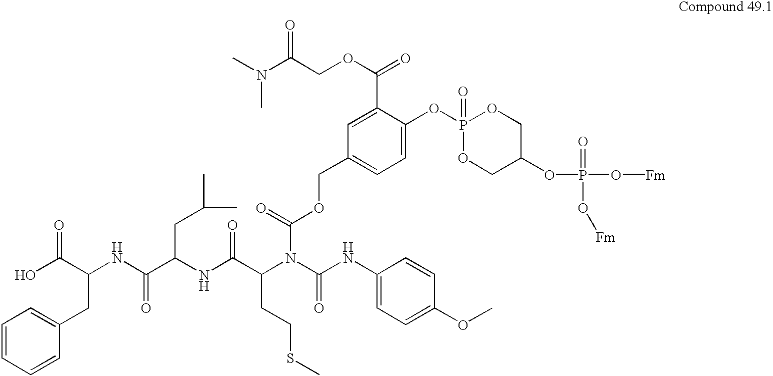

Compound 49.1 may be prepared by deprotection of compound 49.2 with Zn and acid.

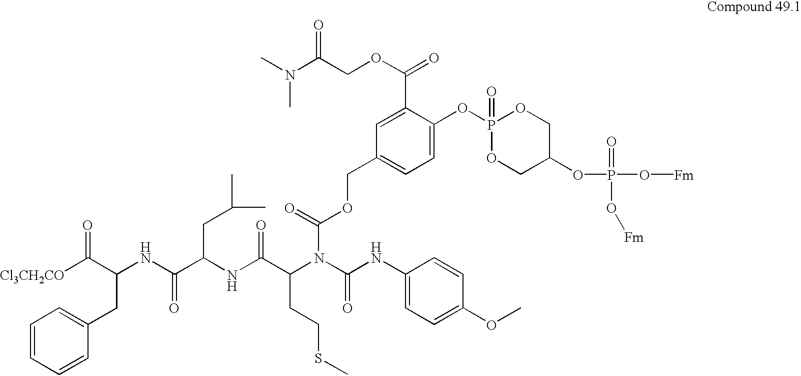

Compound 49.2 may be prepared by a multi-step process. Treating compound 49.3 with acid will remove the t-butyidimethylsilyl protecting group. Treating the product with phosphorochloridic acid bis-(9H-fluoren-9-ylmethyl) ester and a base such as triethylamine in an inert solvent will give compound 49.2.

Compound 49.3 may be prepared by coupling compounds 49.4 and 49.5.

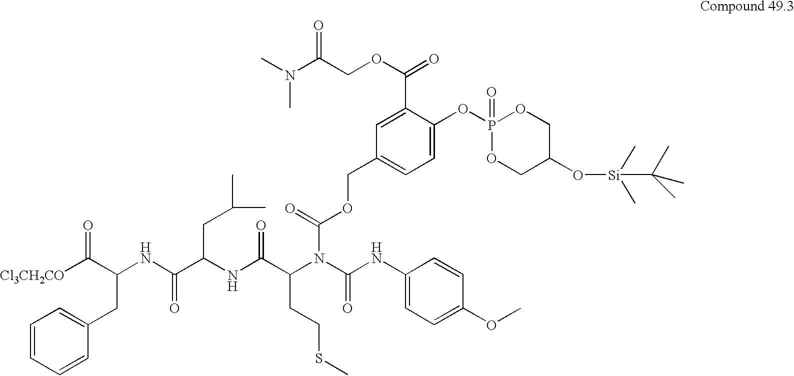

Compound 49.4 may be synthesized from the L-amino acids using routine methods of peptide synthesis. Compound 49.5 may be prepared by treating compound 49.6 with dilute acid to cleave the 1-methyl-1-(4-biphenylyl)ethyl ester group.

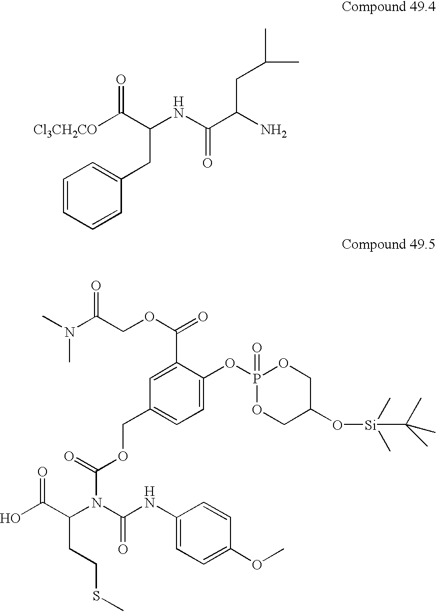

Compound 49.6 may be prepared by treating compound 49.7 with a strong base in an inert solvent at a low temperature such as −78° C. and then reacting with phosgene and p-methoxyaniline.

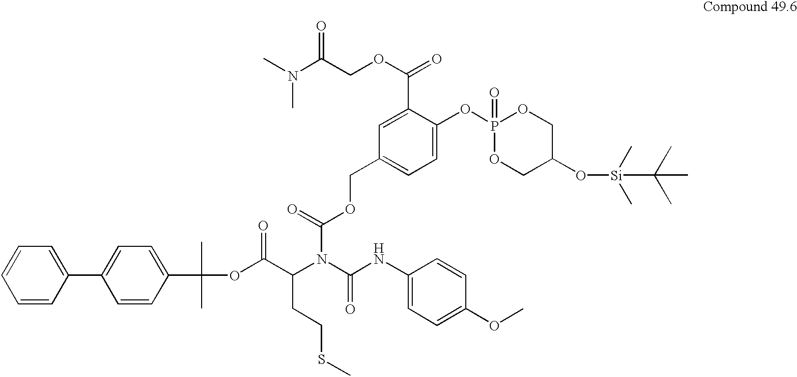

Compound 49.7 may be prepared by reacting L-methionine 1-methyl-1-(4-biphenylyl)ethyl ester with compound 49.8 in an inert solvent in the presence of base. Compound 49.8 may be prepared by treating compound 49.9 with phosgene and base in an inert solvent.

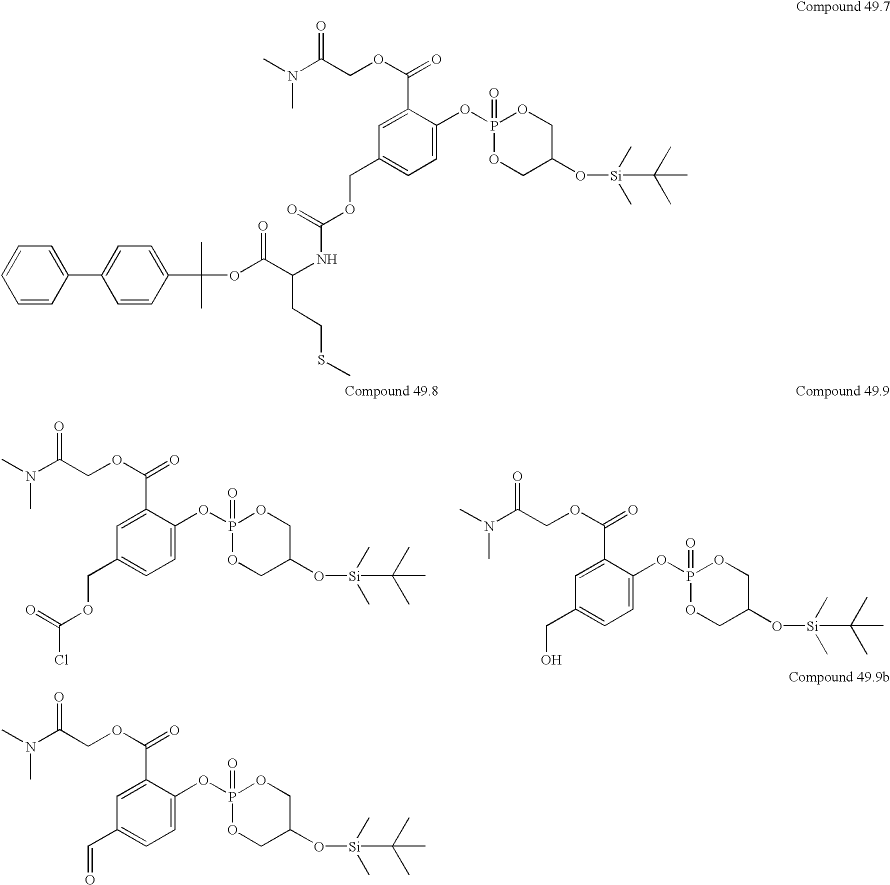

Compound 49.9 may be prepared by reacting compound 33.2 with compound 49.10 in an inert solvent in the presence of base and then reducing the aldehyde (compound 49.9.b) by catalytic hydrogenation with palladium on carbon or by a reagent such as sodium borohydride.

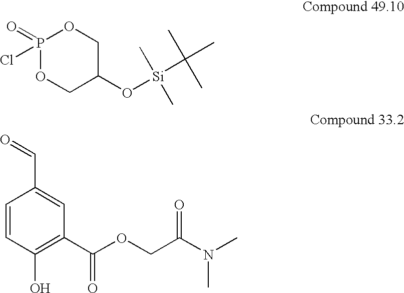

Compound 49.10 may be prepared by reacting phosphorous oxychloride and 2-(tert-Butyl-dimethyl-silanyloxy)-propane-1,3-diol in an inert solvent in the presence of base.

Treating 2-Phenyl-[1,3]dioxan-5-ol with t-butyldimethylchlorosilane and base in an inert solvent and then hydrogenating with palladium on carbon will give -(tert-Butyl-dimethyl-silanyloxy)-propane-1,3-diol.

The neoantigens for sensitization purposes are the same as described for compound 47.

### Example 50

Compound 50 is similar to compound 43, however it has a potent inhibitor of thymidylate synthase 1843U89 to which is attached, by a short linker, a masked hydroxy-salen copper complex. Cleavage of the salen phosphate ester by phosphatase and of the intracellular trigger by thioreductases will free the hydroxy-salen copper-TS inhibitor complex from the remainder of the targeted drug. The complex will bind tightly to TS. Free radicals generated by the copper complex will react with TS and induce neoantigens that may be used for targeted immunotherapy. Salen copper and salen iron complexes are known to generate free radicals under a variety of conditions. The presence of para hydroxy substituents on the salicylidene moieties leads to a radical generating system from oxygen. The hydroxy substituted salicylidene moieties form hydroquinones, which cooperate in the redox reaction and aid in the generation of free radicals. Intracellularly a variety of mechanisms exist that can lead to redox cycling and the continued generation of free radicals. The following references relate to this subject matter: Lamour E., et al., “Oxidation of Cu′ to Cu′″, Free Radical Production, and DNA Cleavage by Hydroxy-Salen-Copper Complexes. Isomeric Effects Studied by ESR and Electrochemisty,” J Am Chem Soc, 121:1862-1869 (1999); Routier S., et al., “DNA Cleavage by Hydroxy-Salicylidene-Ethylendiamine-Iron Complexes,” Nucleic Acids Res, 27(21):4160-4166 (1999); Routier S., et al., “Synthesis of a Functionalized Salen-Copper Complex and Its Interaction with DNA,” J Org Chem, 61:2326-2331 (1996), the contents of which are incorporated herein by reference in their entirety.

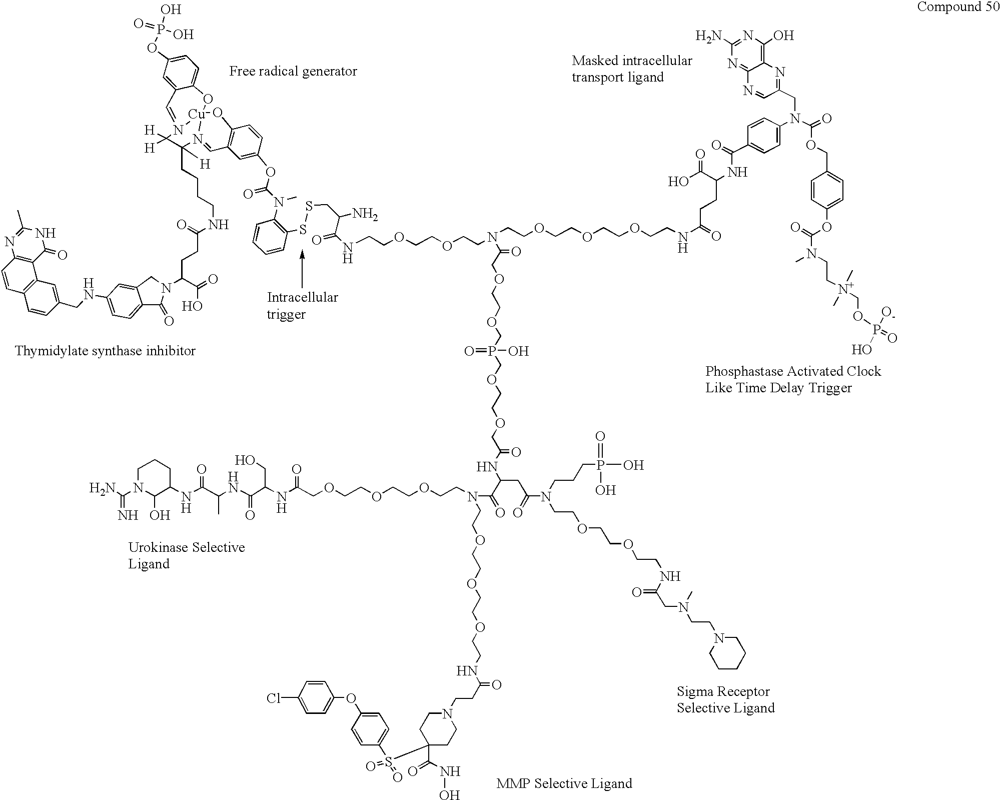

Compound 50 may be prepared by the methods described for compound 38 by replacing compound 21.2.1d with compound 18.1 and also replacing 38.1 with compound 50.1.

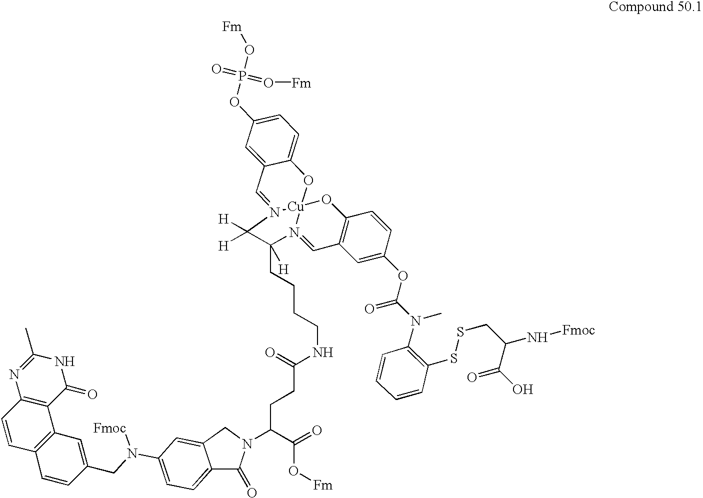

Compound 50.1 may be prepared by coupling compound 50.2 and compound 50.3 and then treating with tris(2-aminoethyl)amine to cleave the Bsm ester under conditions that will leave the Fmoc group intact. The synthesis of compound 50.2 is described in example 8 (compound 8.2.1).

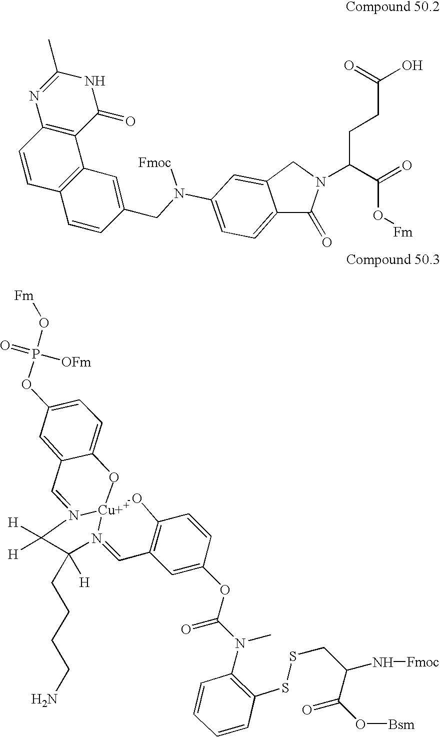

Compound 50.3 may be prepared by reacting compound 50.4 (or compound 50.4.1) and compound 50.5 in an inert solvent in the presence of base and then removing the t-Boc group with acid.

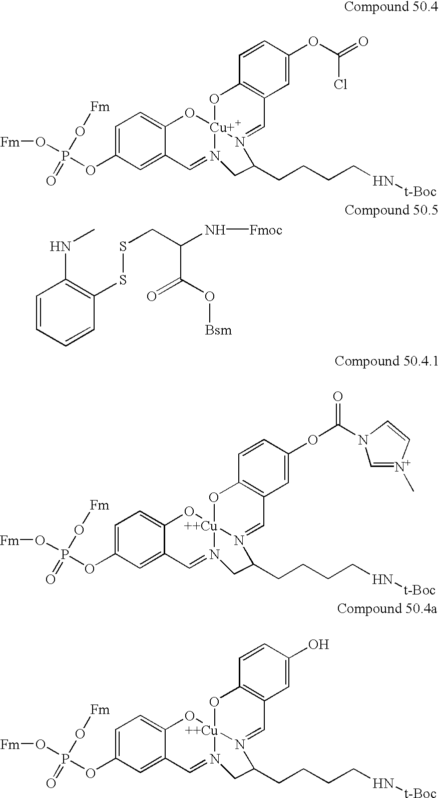

Compound 50.4 may be prepared by treating compound 50.4a with phosgene in an inert solvent.

Compound 50.4.1 may be prepared by treating compound 50.4a with 1,1′-carbonylbis(3-methylimidazolium) triflate in an inert solvent at low temperature in the presence of base. The following references relate to this subject matter: Saha A. K., et al., “1,1′-Carbonylbis(3-methylimidazolium) Triflate: An Efficient Reagent for Aminoacylations,” J Am Chem Soc, 111:4856-4859 (1989), the contents of which are incorporated herein by reference in their entirety.

Compound 50.4a may be prepared by reacting compound 50.4b, compound 50.4c, compound 50.4d, and cooper (II) acetate and isolating the desired product by chromatography.

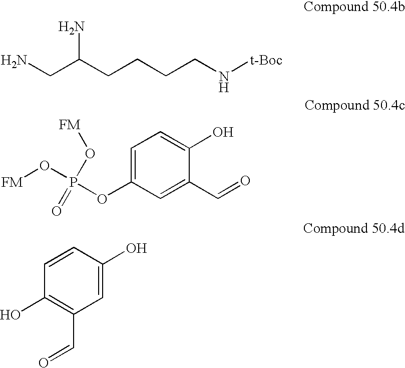

The following references relate to this subject matter: Lamour E., et al., “Oxidation of Cu″ to Cu′″, Free Radical Production, and DNA Cleavage by Hydroxy-Salen-Copper Complexes. Isomeric Effects Studied by ESR and Electrochemisty,” J Am Chem Soc, 121:1862-1869 (1999); Routier S., et al., “Synthesis of a Functionalized Salen-Copper Complex and Its Interaction with DNA,” J Org Chem, 61:2326-2331 (1996), the contents of which are incorporated herein by reference in their entirety.

Compound 50.4c may be prepared by treating 2-(tert-Butyl-dimethyl-silanyloxy)-5-hydroxy-benzaldehyde with phosphorochloridic acid bis-(9H-fluoren-9-ylmethyl) ester and a base such as triethylamine in an inert solvent and then removing the silyl protecting group with acid.

### Example 51

Compound 51 is similar to compound 50, however the hydrox-salen copper complex is masked as phosphate esters, which will be cleaved by phosphatases to activate the free radical generator. The TS inhibitor-salen-copper complex will be freed from the remainder of the drug by an intracellular trigger following activation by intracellular thioreductases.

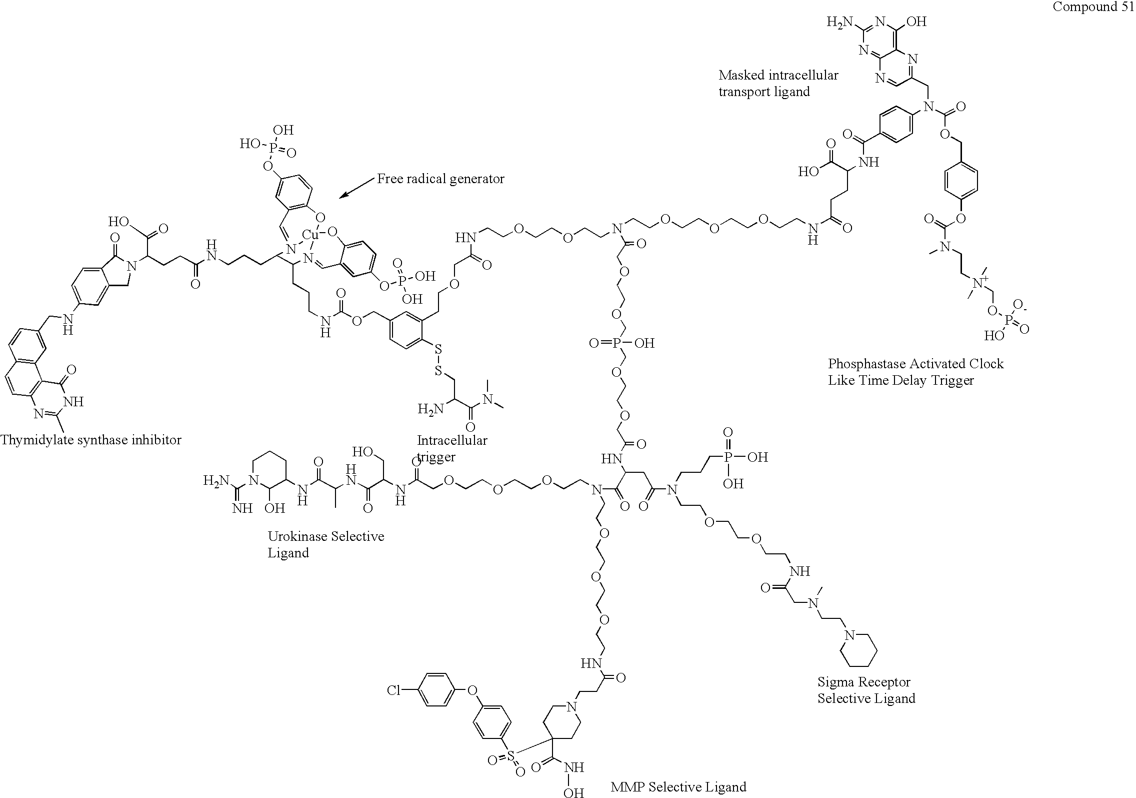

Compound 51 may be prepared by the method described for compound 50 by replacing compound 50.1 with compound 51.1.

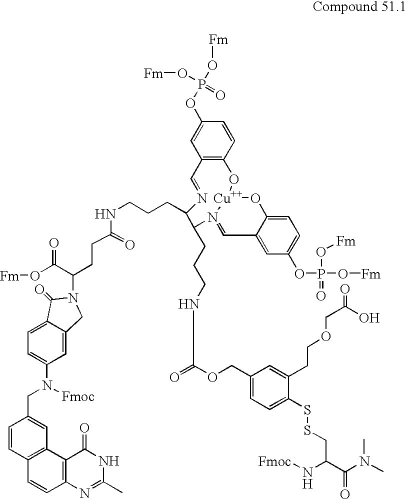

Compound 51.1 may be prepared by coupling compound 50.2 and compound 51.2 and then treating with tris(2-aminoethyl)amine to cleave the Bsm ester under conditions that will leave the Fmoc group intact.

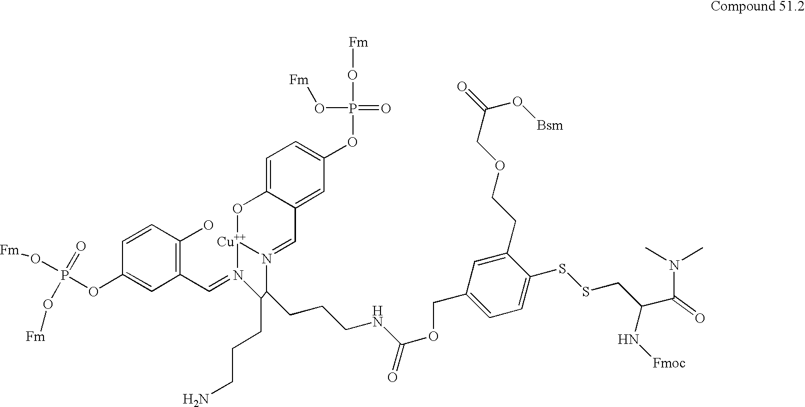

Compound 51.2 may be prepared by reacting compound 51.3 and compound 51.4 in an inert solvent in the presence of base.

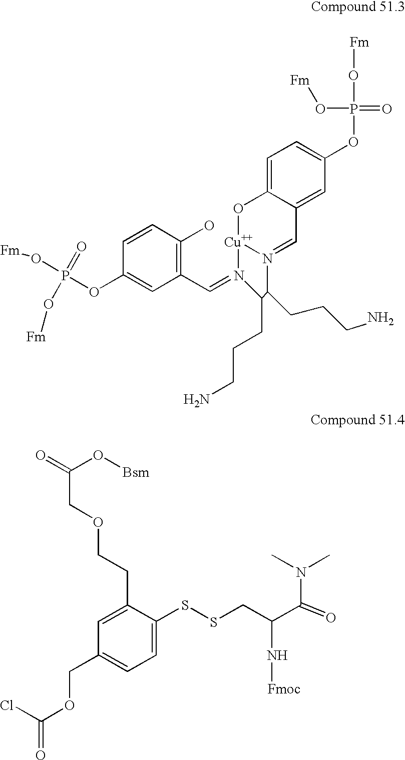

Compound 51.3 may be prepared by reacting 2 equivalents of compound 50.4c, compound 51.5 and copper (11) acetate and then treating with acid to remove the t-Boc groups.

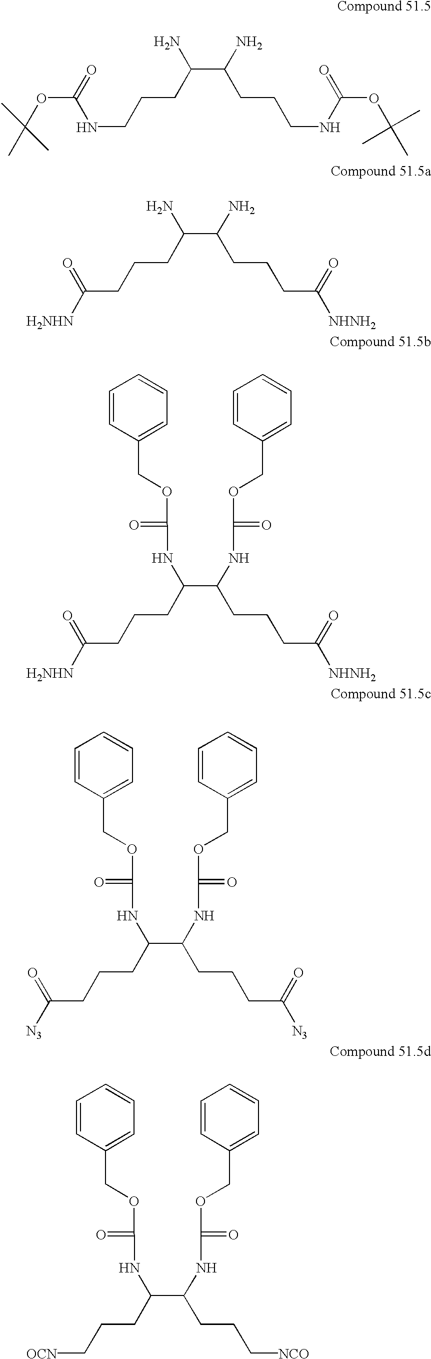

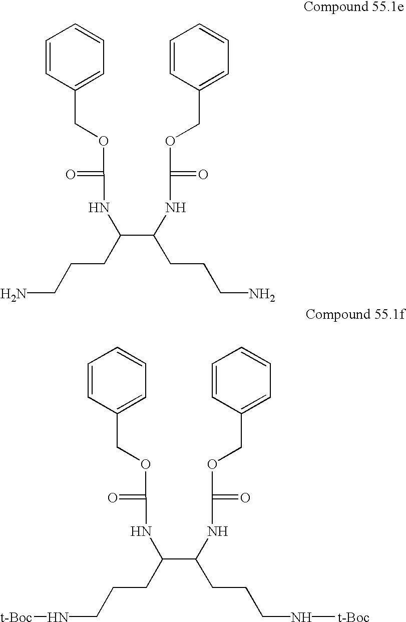

Compound 51.5 may be prepared by a multi-step process. Catalytic hydrogenation of [2,2′]Bipyridinyl-6,6′-diol with Pd on carbon will give [2,2′]Bipiperidinyl-6,6′-dione. Treatment with hydrazine will give compound 51.5a. Reaction with 2 equivalents of benzyl chloroformate and base will protect the more reactive amino groups and give compound 51.5b. Treatment with sodium nitrite and acid will give compound 51.5c. Heating will, via the Curtius rearrangement, give compound 51.5d. Hydrolysis will give compound 51.5e. Treatment with di-t-butyl pyrocarbonate and in an inert solvent will give compound 51.5f. Catalytic hydrogenation will give compound 51.5.

Compound 51.4 may be prepared by the methods described for the synthesis of compound 23.2b by replacing compound 14.11.4 with L-N-Fmoc-cysteine N,N-dimethylamide.

The neoantigens, for sensitization purposes, may be prepared by treating thymidylate synthase with a compound such as compound 51.6 in the presence of oxygen and a reducing agent such as ascorbic acid so that a redox cycle can be established leading to augmented hydroxy radical production. Alternatively, the compounds derived from the interaction of TS and compound 51.6 may be identified, synthesized and employed.

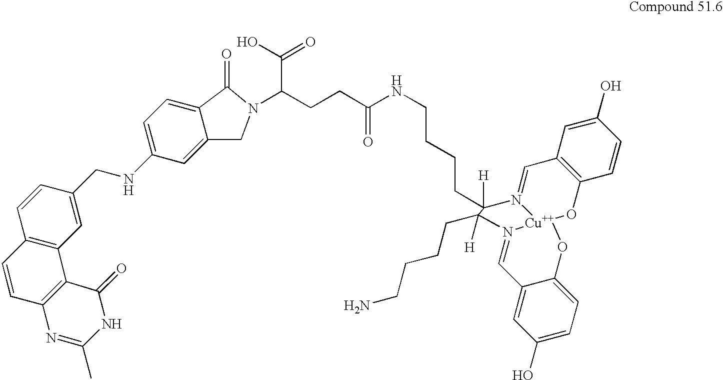

### Example 52

Compound 52 is similar to compound 51, however, an enediyne analog is employed to generate free radicals and create neoantigens from the enzyme TS. The following references relate to this subject matter: Zein N., et al., “Protein Damage Caused by a Synthetic Enediyne Core,” Biorg Med Chem Leff, 3(6):1351-1356 (1993); Kadow J. F., et al., “Conjugate Addition-Aldol Approach to the Simple Bicyclic-Diynene Core Structure Found in the Esperamicins and Calicheamicins,” Tetrahedron Lett, 33(11):1423-1426 (1992); U.S. Pat. No. 5,395,849 Mar. 7, 1995 Wittman, et al., “Hybrid Antitumor Compounds Containing a Cyclic Enediyne and a DNA-Binder”; U.S. Pat. No. 5,198,560 Mar. 30, 1993 Kadow, et al., “Cytotoxic Bicyclo[7.3.1]Tridec-4-Ene-2,6-Diyne Compounds and Process for the Preparation Thereof”, the contents of which are incorporated herein by reference in their entirety.

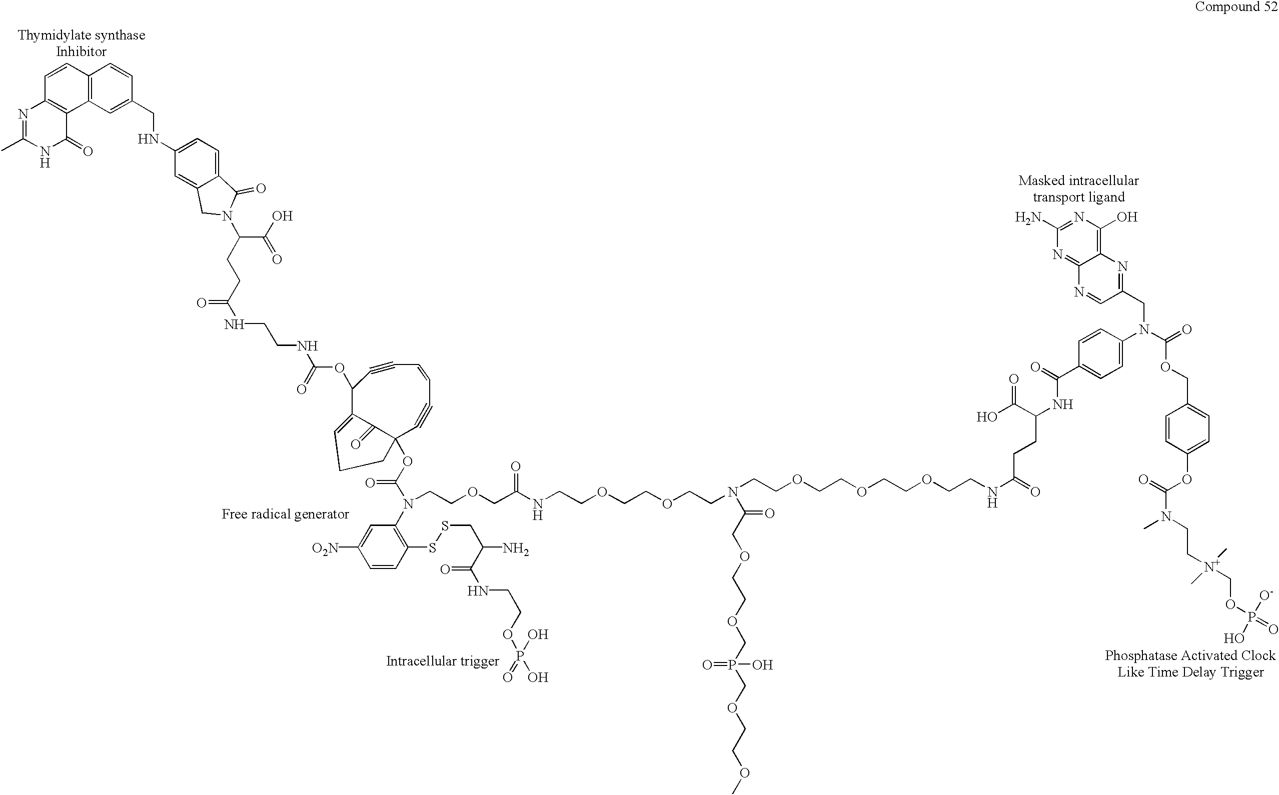

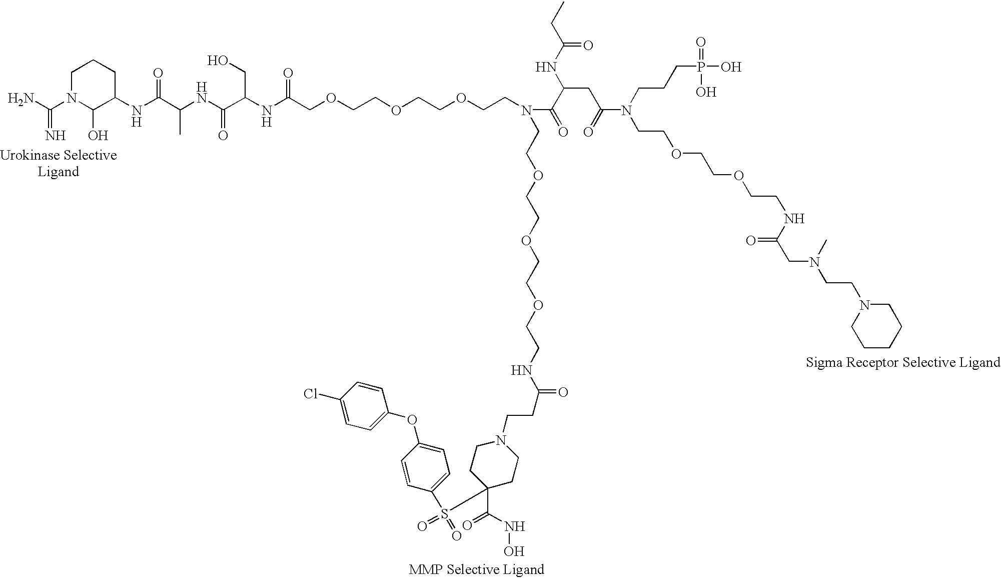

Compound 52 may be prepared by the method described for compound 50 by replacing compound 50.1 with compound 52.1. Also, the order of deprotection of compound 21.1.1 should be modified, as Bsm deprotection with tris(2-aminoethyl)amine may not be compatible with preservation of the enediyne. Compound 21.1.1 should have the Bsm group removed prior to reacting with compound 52.1. This leaves a free carboxylate group and requires that compound 31.1 to be converted into an active ester such as an N-hydroxysuccinimide ester for its coupling reaction to avoid unwanted side products.

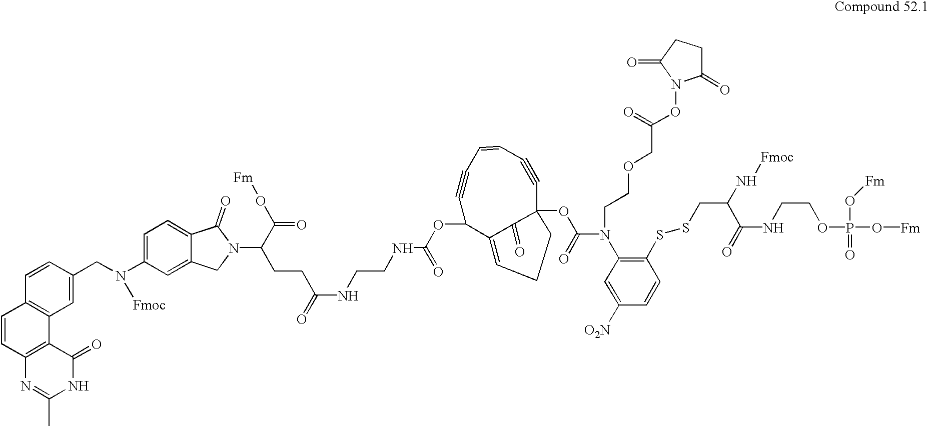

Compound 52.1 may be prepared in a multi-step process. Compound 52.2 may be treated with phosgene in an inert solvent at low temperature and then reacted with (2-Amino-ethyl)-carbamic acid tert-butyl ester to give compound 52.3. Selective removal of the t-butyidimethylsilyl protecting group with acid followed by treatment with phosgene in an inert solvent will give compound 52.4.

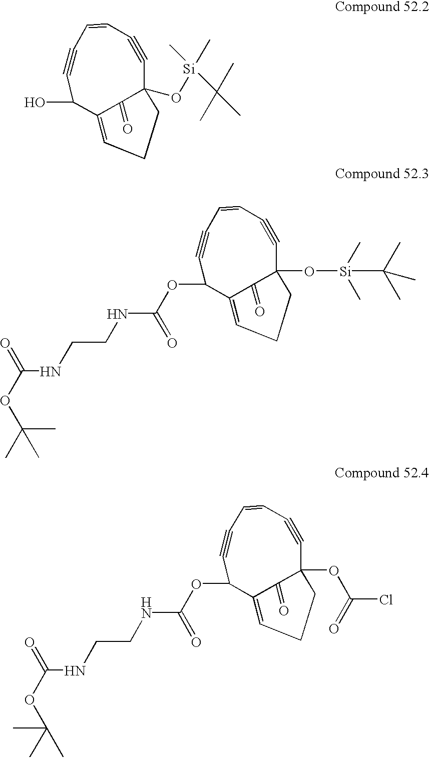

Compound 52.4 may be reacted with compound 52.5 in an inert solvent in the presence of base. Treatment of the product with acid will cleave the t-Boc and t-butyl ester groups and give compound 52.6.

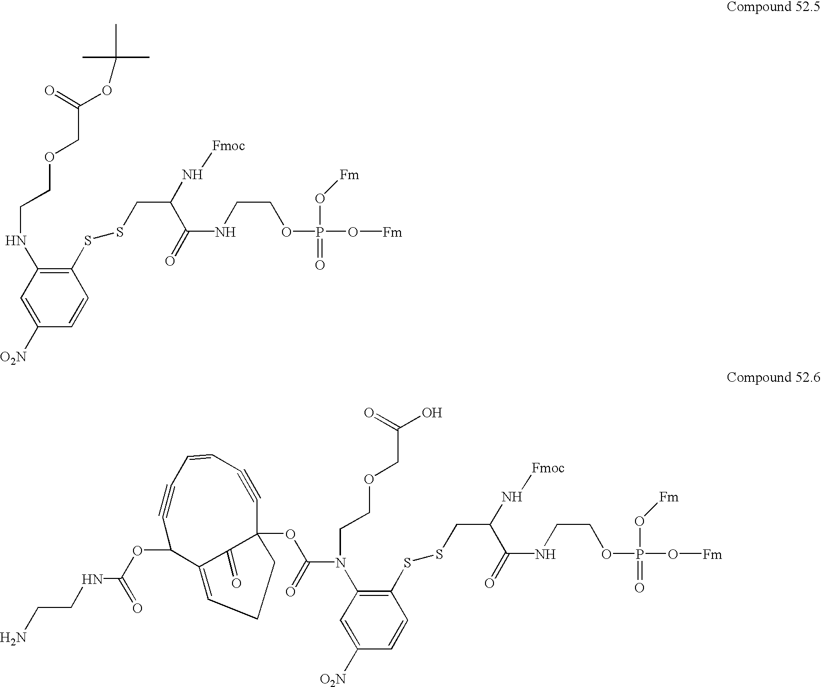

Compound 52.6 may then be reacted with compound 52.7. Treatment of the product with a reagent such as dicylcohexylcarbodiimide and N-hydroxysuccinimide will give compound 52.1.

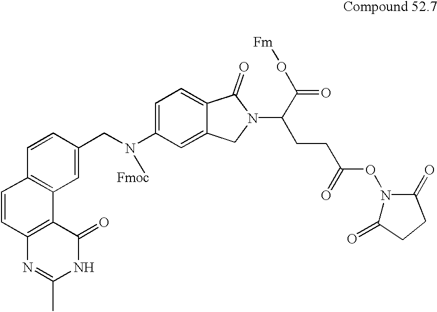

Compound 52.7 may be prepared by coupling of compound 50.2 with N-hydroxysuccinimide using a reagent such as dicyclohexylcarbodiimide.

The neoantigens, for sensitization purposes, may be prepared by treating TS with a compound, such as compound 52.8 in the presence of a thiol, such as cysteine to initiate diradical formation. Alternatively, the synthetic oligopeptides that correspond to the family of TS derived products generated by the interaction of activated compound 52.8 and TS may be employed.

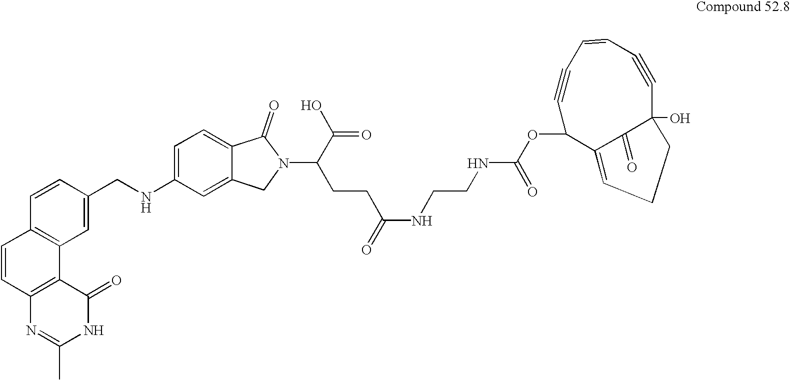

### Example 53

Compound 53 is similar to compound 52, however in a chelate of Iron (II) is employed as a free radical generator to create neoantigens from TS. Iron (II) complexes with chelating agents are known to generate free radicals under a variety of conditions. The following references relate to this subject matter: Kocha T., et al., “Hydrogen Peroxide-mediated Degradation of protein: Different Oxidation Modes of Copper- and Iron-dependent Hydroxyl Radicals on the Degradation of Albumin,” Biochem Biophys Acta, 1337:319-326 (1997); Egan T. J., et al., “Catalysis of the Haber-Weiss Reaction by Iron-Diethylenetriaminepentaacetate,” J Inorg Biochem, 48:241-249 (1992); Hertzberg R. P.; Dervan P. B., “Cleavage of DNA with Methidiumpropyl-EDTA-Iron (II): Reaction Conditions and Product Analyses,” Biochemistry, 23:3934-3945 (1984); Schepartz A.; Cuenoud B., “Site-Specific Cleavage of the Protein Calmodulin Using a Trifluoperazine-Based Affinity Reagent,” J Am Chem Soc, 112:3247-3249 (1990), the contents of which are incorporated herein by reference in their entirety.

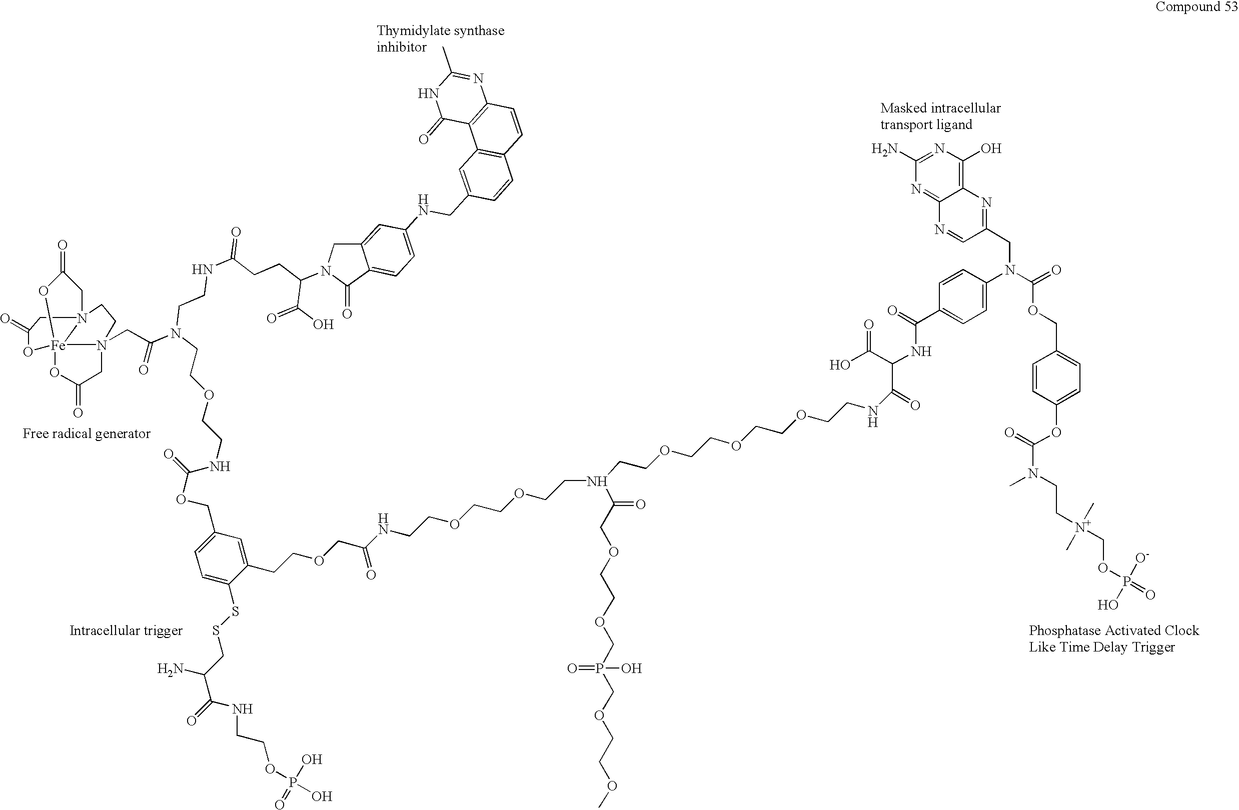

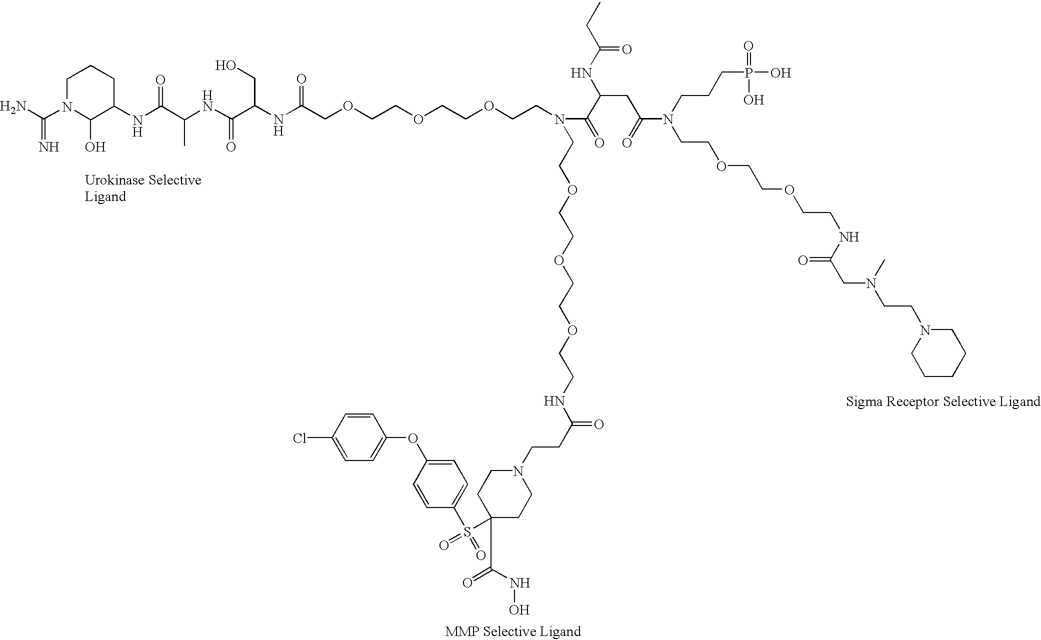

Compound 53 may be prepared as described for compound 50 by replacing compound 50.1 with compound 53.1.

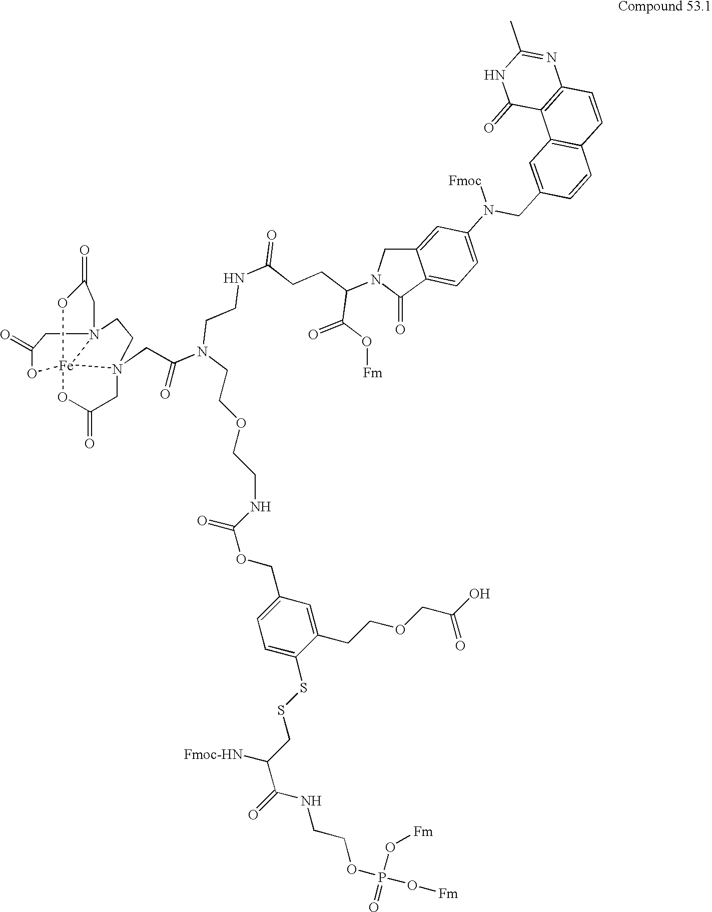

Compound 53.1 may be prepared by reacting compound 53.2 and compound 23.2b in an inert solvent, in the presence of base, and then treating with tris(2-aminoethyl)amine to cleave the Bsm ester under conditions that will leave the Fmoc groups intact.

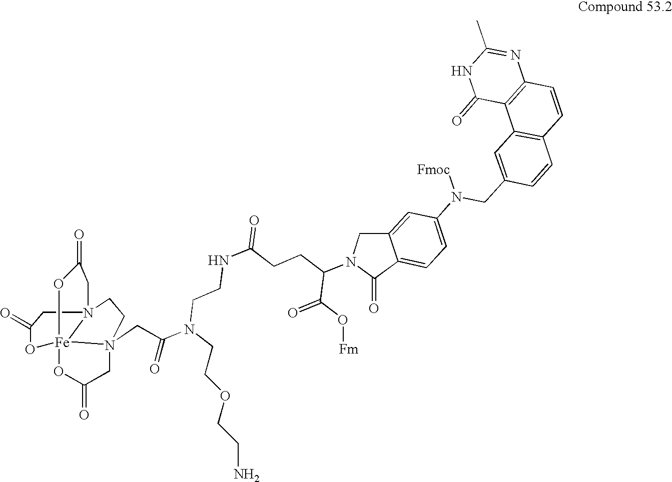

Compound 53.2 may be prepared by reacting compound 53.3 and compound 52.7 and then treating with acid to remove the t-Boc group.

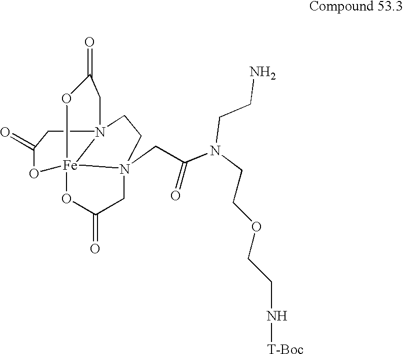

Compound 53.3 may be prepared by a multi-step procedure. Coupling compound 53.4 and compound 53.5 will give compound 53.6. Hydrolysis of the ethyl esters and treatment with an Iron (II) salt will give compound 53.7. The trityl protecting group may then be selectively removed by treatment with acid to give compound 53.3.

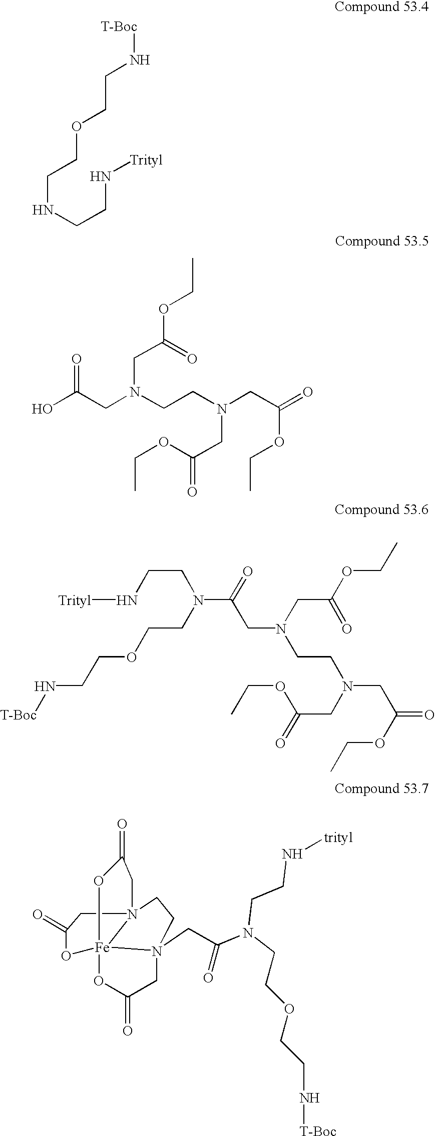

Compound 53.4 may be prepared by reacting benzenesulfonic acid 2-(2-tert-butoxycarbonylamino-ethoxy)-ethyl ester and N1-Trityl-ethane-1,2-diamine in an inert solvent in the presence of base.

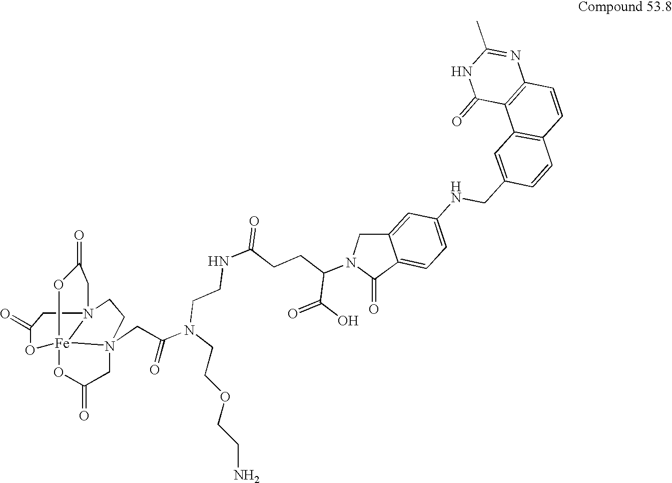

Neoantigens, for sensitization purposes, may be prepared by treating TS with a compound such as compound 53.8 in the presence of hydrogen peroxide, or hydrogen peroxide and ascorbic acid, or with a thiol base reducing agent under aerobic conditions. Alternatively, synthetic oligopeptides corresponding to the degradation products, resulting from the interaction of compound 53.8 with TS under Fenton conditions may be employed.

### Example 54

Compound 54 is similar to compounds 34 and compound 53. Compound 54 will generate neoantigens from epidermal growth factor receptors by both covalent modification of the active site of the enzyme and by Fenton chemistry induced free radical damage to the enzyme.

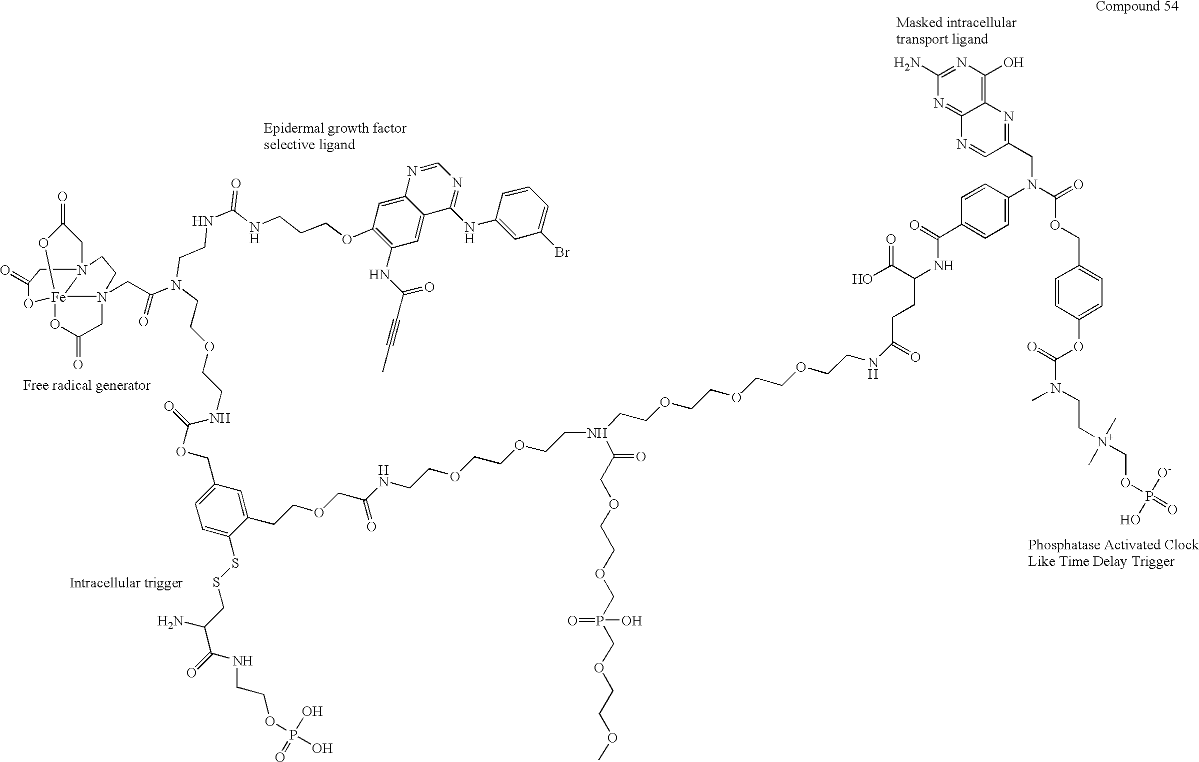

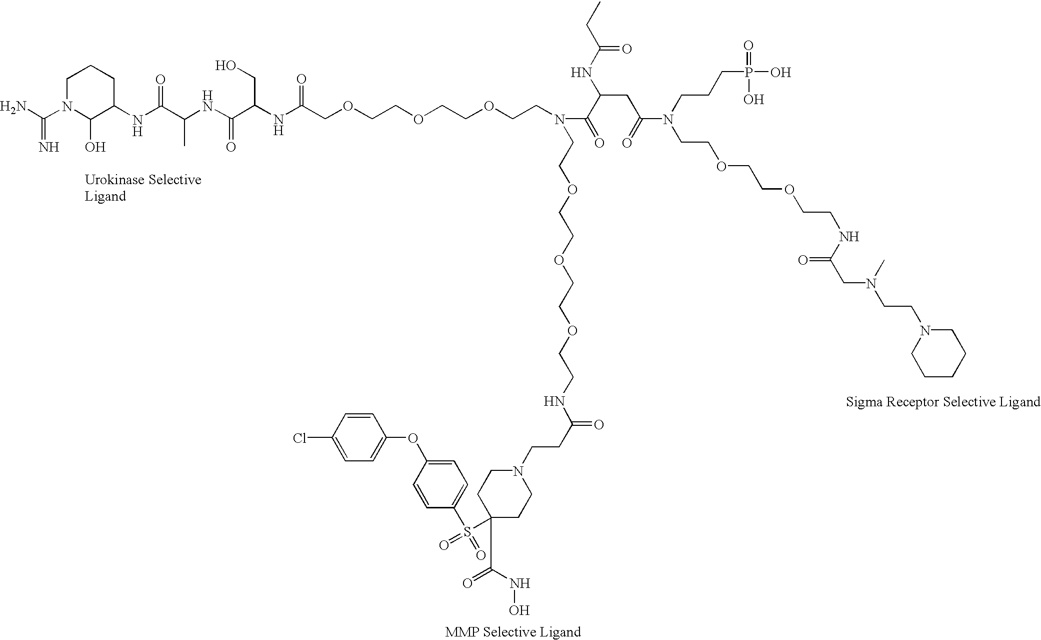

Compound 54 may be prepared by the method described for compound 53 by replacing compound 53.2 with compound 54.1.

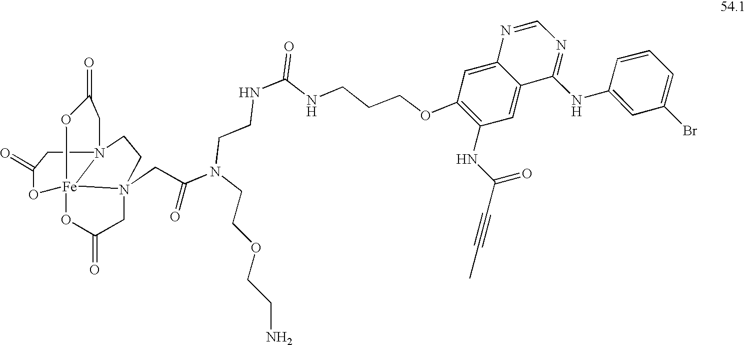

Compound 54.1 may be prepared by reacting compound 34.2 and compound 54.2 and then treating with acid to remove the t-Boc group.

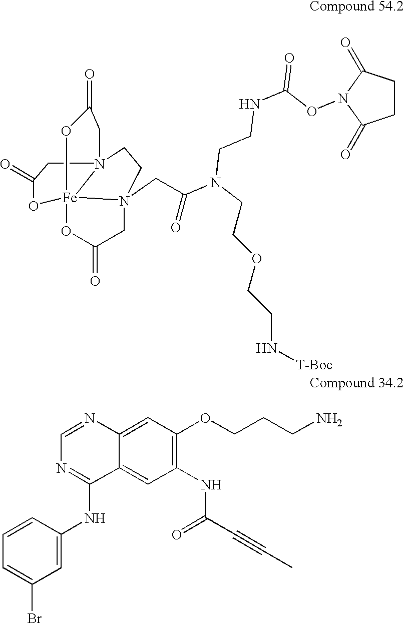

Compound 54.2 may be prepared by treating compound 53.3 with N,N′, disuccinimidyl carbonate in an inert solvent in the presence of pyridine.

The neoantigens, for sensitization purposes, may be prepared by treating the respective epidermal growth factor related target with a compound such as compound 54.1 under conditions as described in example 53. Alternatively, the corresponding synthetic oligopeptides may be employed.

### Example 55

Compounds A55 and B55 are a set of monofactorial drugs that will exhibited target synergistic toxicity and multifactorial targeting when administered in combination. Compound A55 will deliver a thymidylate synthase inhibitor to prostate specific membrane antigen positive cells. Compound B55 will deliver a nucleoside transport inhibitor to urokinase positive cells. Prostatic cancer cells that jointly express both urokinase and PSMA will have both denovo and salvage pathways of thymidine metabolism inhibited and will be selectively killed.

Compound A55 has a PSMA targeting ligand, a fatty amide ligand that will bind nonspecifically, but weakly to cell membranes and a masked intracellular transport ligand with an esterase activated time delay clock like trigger. A potent inhibitor of TS (1843U89) will be released upon activation of an intracellular trigger. The intracellular trigger may be activated either by reduction of the quinone, by DT-diaphorase, or by nucleophilic activation by glutathione. The following references relate to this subject matter: Flader C., et al., “Development of Novel Quinone Phosphorodiamidate Prodrugs Targeted to DT-Diaphorase,” J Med Chem, 43:3157-3167 (2000), the contents of which are incorporated herein by reference in their entirety.

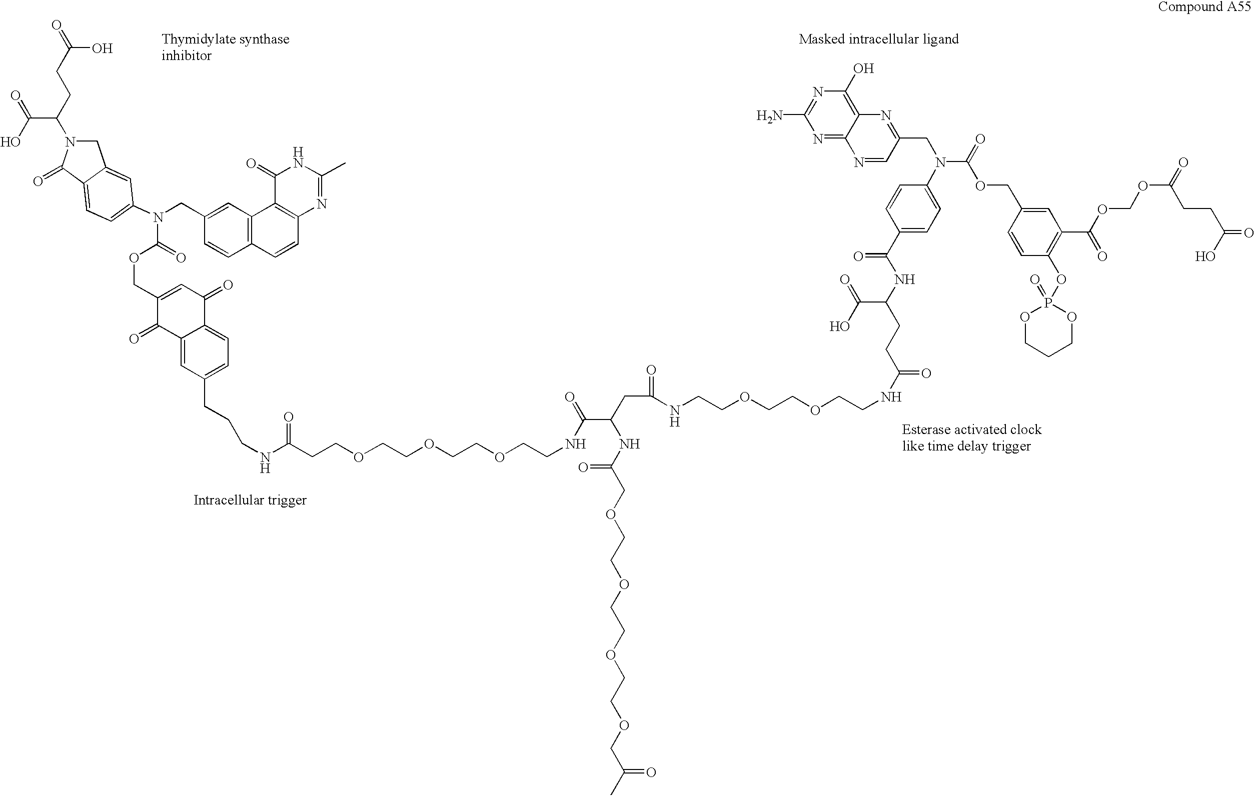

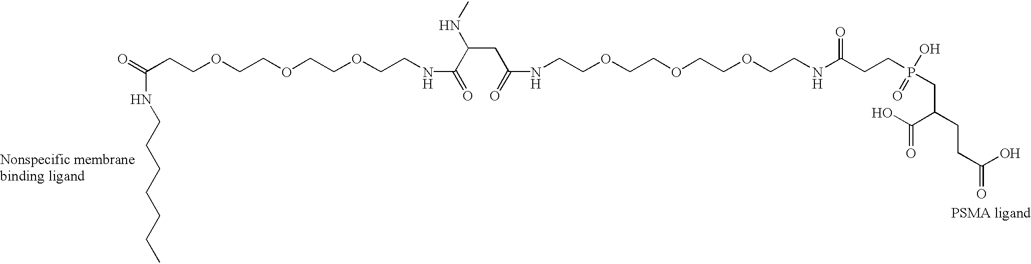

Compound A55 may be prepared by a multi-step process. Compounds A55.1 and A55.2 may be coupled to give compound A55.3.

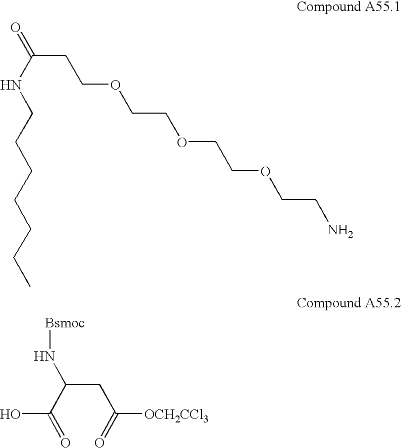

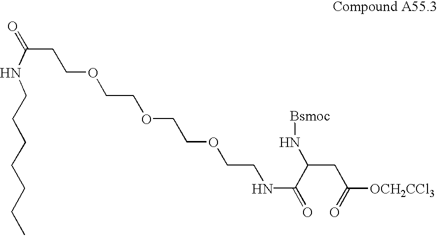

Treatment with Zn and acid, followed by coupling to compound 44.3, followed by removal of the Bsmoc protecting group with tris(2-aminoethyl)amine under conditions that will leave the Fmoc group intact, will give compound A55.4.

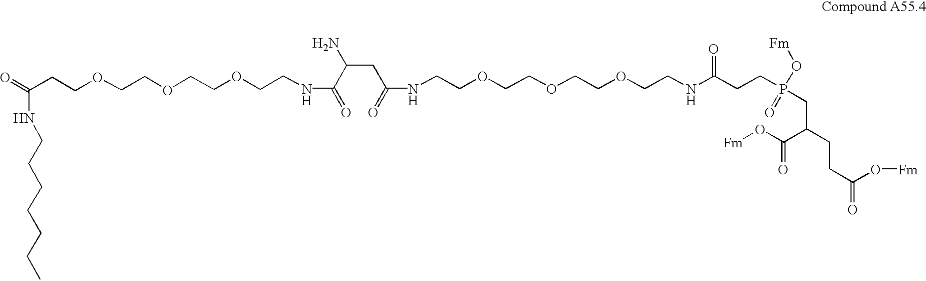

Coupling compound A55.4 and (2-{2-[2-(2,2,2-Trichloroethoxycarbonyl methoxy)-ethoxy]-ethoxy}-ethoxy)-acetic acid followed by treatment with Zn and acetic acid will give compound A55.5.

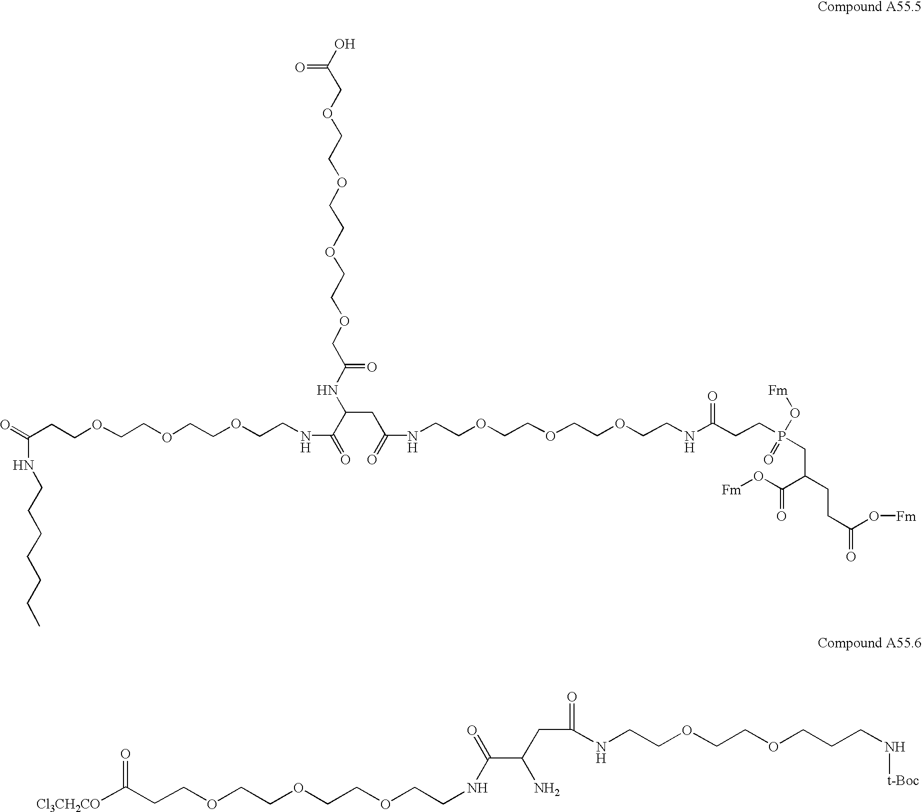

Coupling compound A55.5 and compound A55.6 and treating with acid to remove the T-boc group will give compound A55.7.

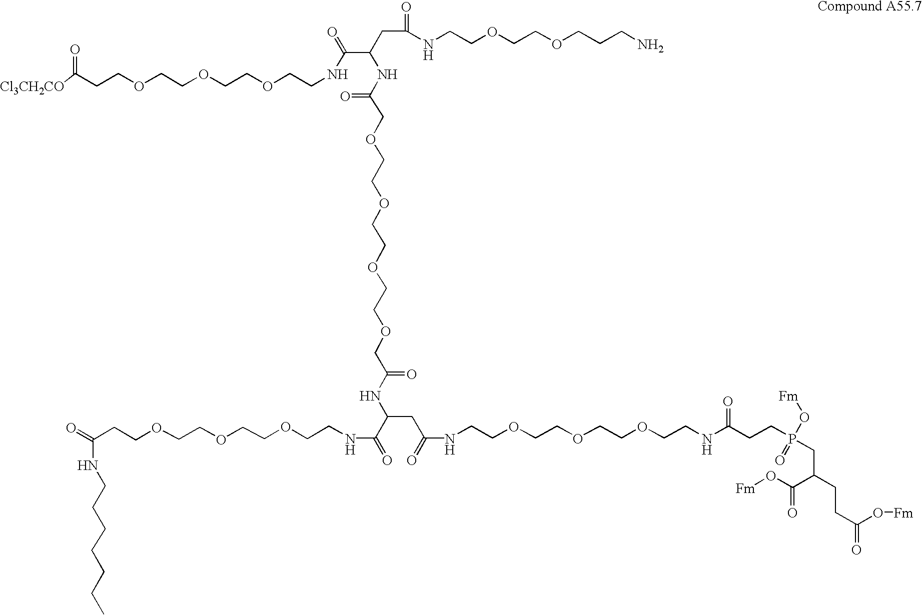

Coupling compound A55.7 and compound 32.1, followed by the removal of the trichloroethyl protecting group with Zn and acetic acid, will give compound A55.8.

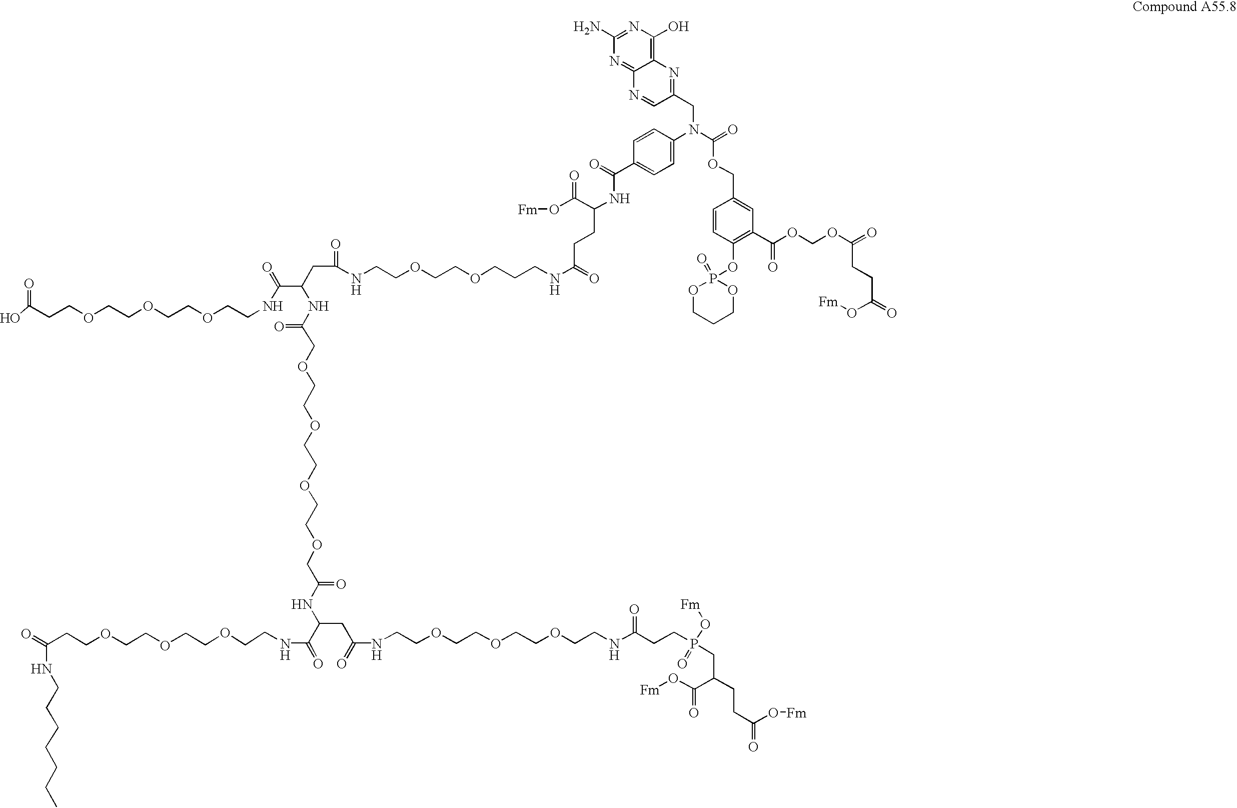

Coupling compound A55.8 with compound A55.9 followed by treatment with base to remove the Fmoc and Fm groups will give compound A55.

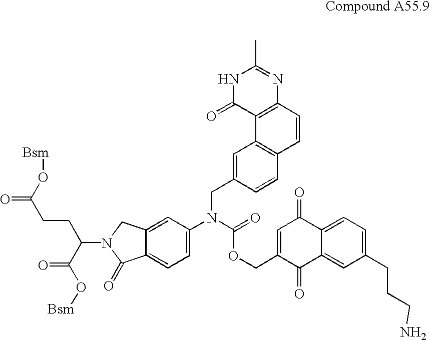

Compound A55.9 may be prepared by reacting compound A55.10 and A55.11 in an inert solvent in the presence of a base such as pyridine and then treating with a base such as diisopropylethylamine to selectively remove the Fmoc group without cleaving the Bsm esters.

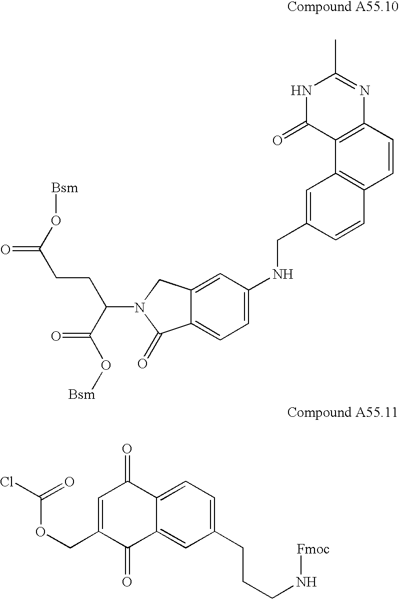

Compound A55.10 may be prepared by a multi-step process. The TS inhibitor 1843U89 may be treated with an excess of a reagent such as hexamethyidisilazane and a catalytic amount of chlorotrimethylsilane in an inert solvent. The following references relate to this subject matter: Duch D. S., et al., “Biochemical and Cellular Pharmacology of 1843U89, a Novel Benzoquinazoline Inhibitor of Thymidylate Synthase,” Cancer Res, 53:810-818 (1993); Pendergast W., et al., “Benzo[f]quinazoline Inhibitors of Thymidylate Synthase: Methyleneamino-Linked Aroylglutamate Derivatives,” J Med Chem, 37:838-844 (1994), the contents of which are incorporated herein by reference in their entirety.

The product may then be reacted with benzyl chloroformate, followed by oxalyl chloride and catalytic amount of dimethylformamide. The resulting acid chloride may then be reacted with (1,1-Dioxo-1H-1λ6-benzo[b]thiophen-2-yl)-methanol and base. Catalytic hydrogenation, with palladium on carbon, will remove the benzyloxycarbonyl protecting group and give compound A55.10. In an alternate method, compound A55.9 may be prepared by reacting compound A55.11b and compound A55.10 in an inert solvent in the presence of a base such as pyridine and oxidizing of the product with a reagent such as cerium ammonium nitrate, and then removing the Fmoc group with diisopropylethylamine.

Compounds A55.11 and A55.1 lb may be prepared by reacting compounds A55.12 and A55.12b respectively with phosgene in an inert solvent.

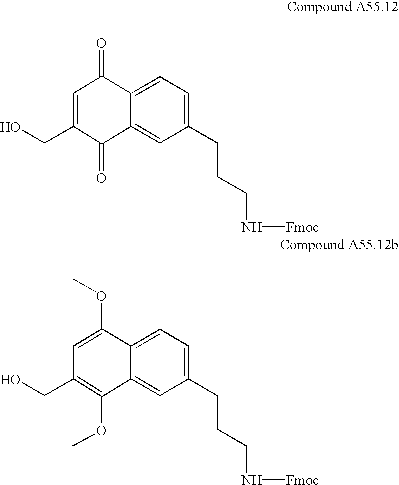

Compound A55.12 may be prepared by a mult-step process. The compound 4-(2-chloroethyl)acetophenone may be converted into 2-[4-(2-Chloro-ethyl)-phenyl]-2-methyl-[1,3]dioxolane by treatment with acid and ethylene glycol. Treatment with sodium cyanide will give 3-[4-(2-Methyl-[1,3]dioxolan-2-yl)-phenyl]-propionitrile. Acid hydrolysis will give 3-(4-Acetyl-phenyl)-propionamide. Treament with cooper (II) chloride will give compound A55.13.

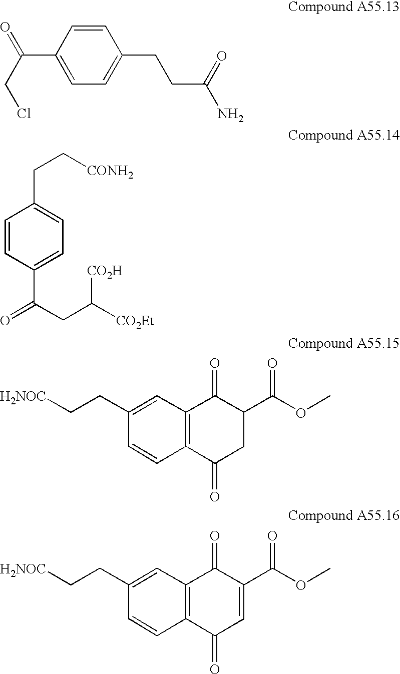

Treatment of A55.15 with diethylmalonate and a strong base in an inert solvent, followed by hydrolysis of one of ethyl ester groups with base, will give compound A55.14. Treatment of compound A55.14 with anhydrous HF will give compound A55.15. The following references relate to this subject matter: Fieser L. F.; Hershberg E. B. “Inter- and Intramolecular Acylations with Hydrogen Fluoride,” J Am Chem Soc, 61:1272-1281 (1939), the contents of which are incorporated herein by reference in their entirety.

A55.15 may be halogenated with an agent such as bromine or copper (II) chloride and then the product should be treated with base to give the elimination product A55.16. Catalytic hydrogenation with Pd on carbon, followed by treatment with methyl iodide and base, will give compound A55.17. Reduction with lithium aluminum hydride in an inert solvent will give compound A55.18. Treatment with 9-fluorenylmethyl chloroformate in an inert solvent will give compound A55.12.b.

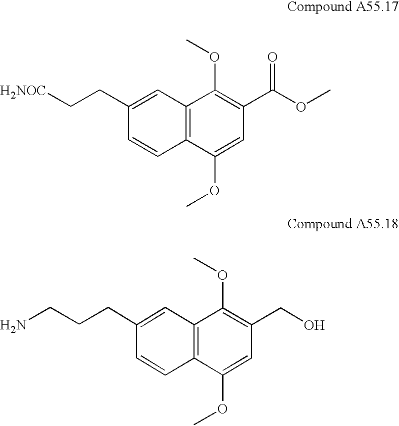

Treatment of compound A55.12b chloro-acetic anhydride and base will give compound A55.19.

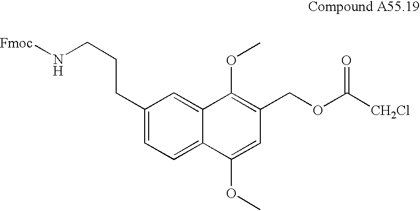

Oxidation with cerium (IV) ammonium nitrate in an inert solvent followed by hydrolysis of the chloroacetate ester will give compound A55.12. .1

Compound B55 has a targeting ligand for urokinase, a low affinity nonspecific membrane binding ligand, and two masked “5′-S-(2Aminoethyl)-N6-(4-Nitrobenzyl)-5′-Thioadenosine ligands, which when unmasked, will bind tightly to nucleoside transport inhibitors on the surface of targeted cells. The nucleoside transport inhibitors are masked by an esterase activated time delay clock like trigger. Its expected rate-limiting step will be the intramolecular nucleophilic attack of the carboxylate group on the phosphotriester, which should proceed with a half life of approximately 90 minutes. The unmasked phenolic hydroxy group, which is in equilibrium with the powerfully electron donating oxyanion, will trigger rapid acetal cleavage by stabilizing carbocation formation at the benzylic carbon.

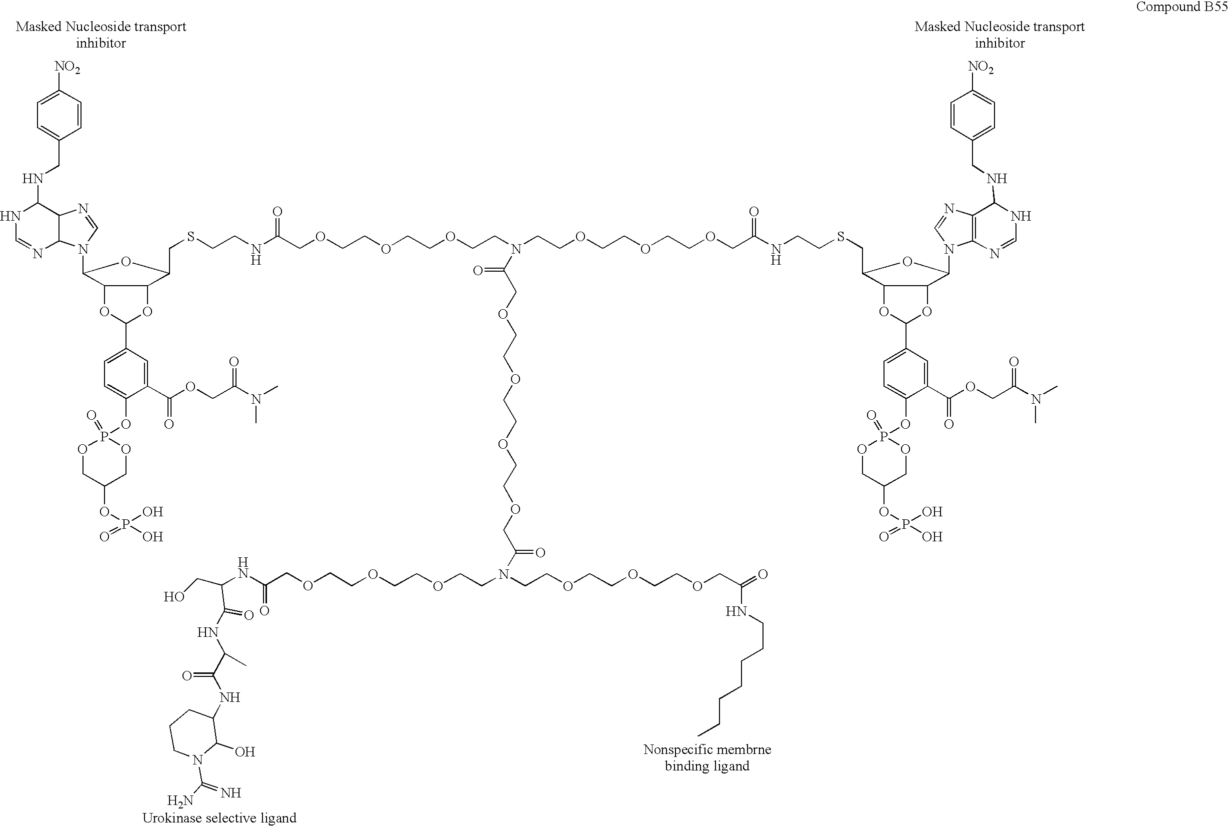

Compound B55 may be prepared by a multi-step process. Treating compound B55.1 with base to cleave the Fm ester, followed by coupling to heptylamine, followed by acid treatment to cleave the t-butyl ester, followed by coupling to compound B55.2 will give compound B55.3. Cleavage of the allyl esters with Pd (0) and coupling to compound B55.4 will give B55.5. Treatment with base will remove the Fmoc and Fm protecting groups. The t-butyidimethylsilyl protecting groups may then be removed with a reagent such as t-butylammonium fluoride or by treatment with acid under conditions that do not cleave the acetal groups to give compound B55.

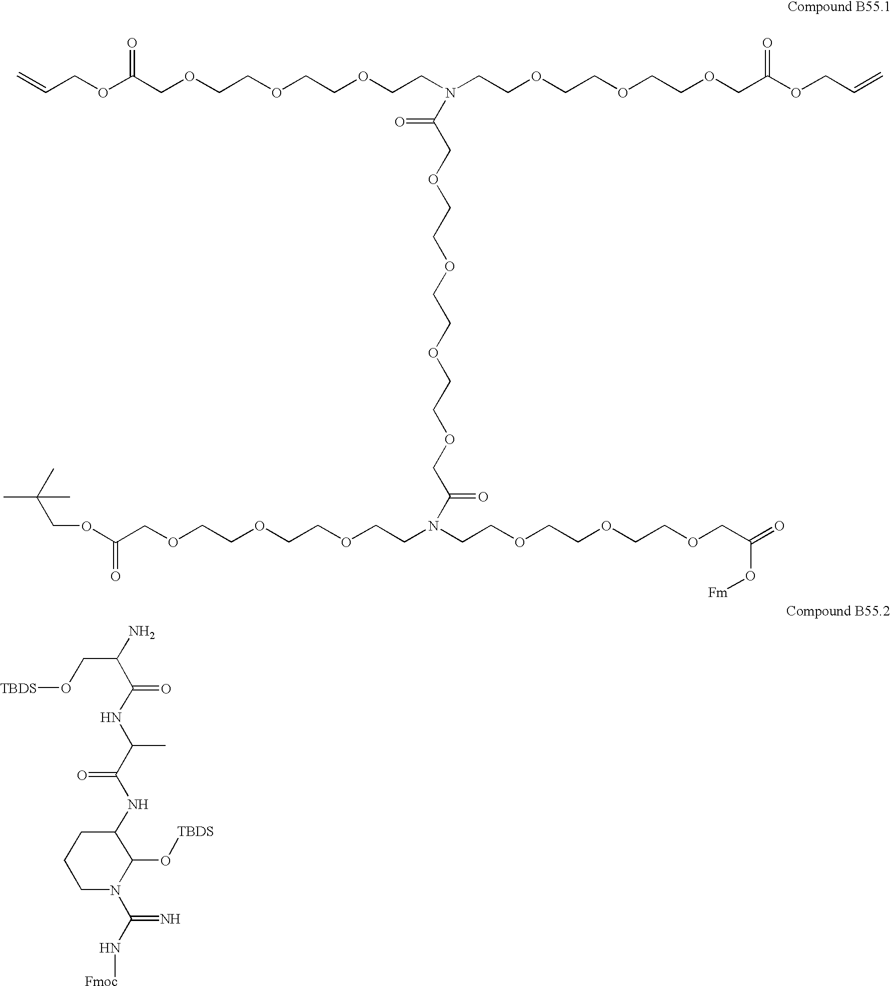

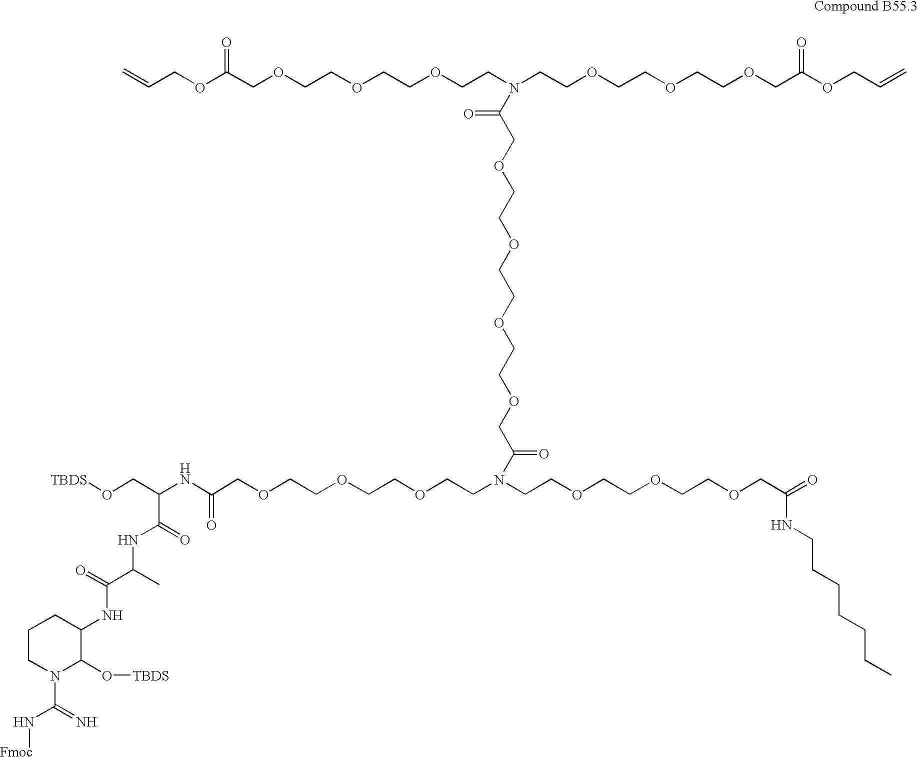

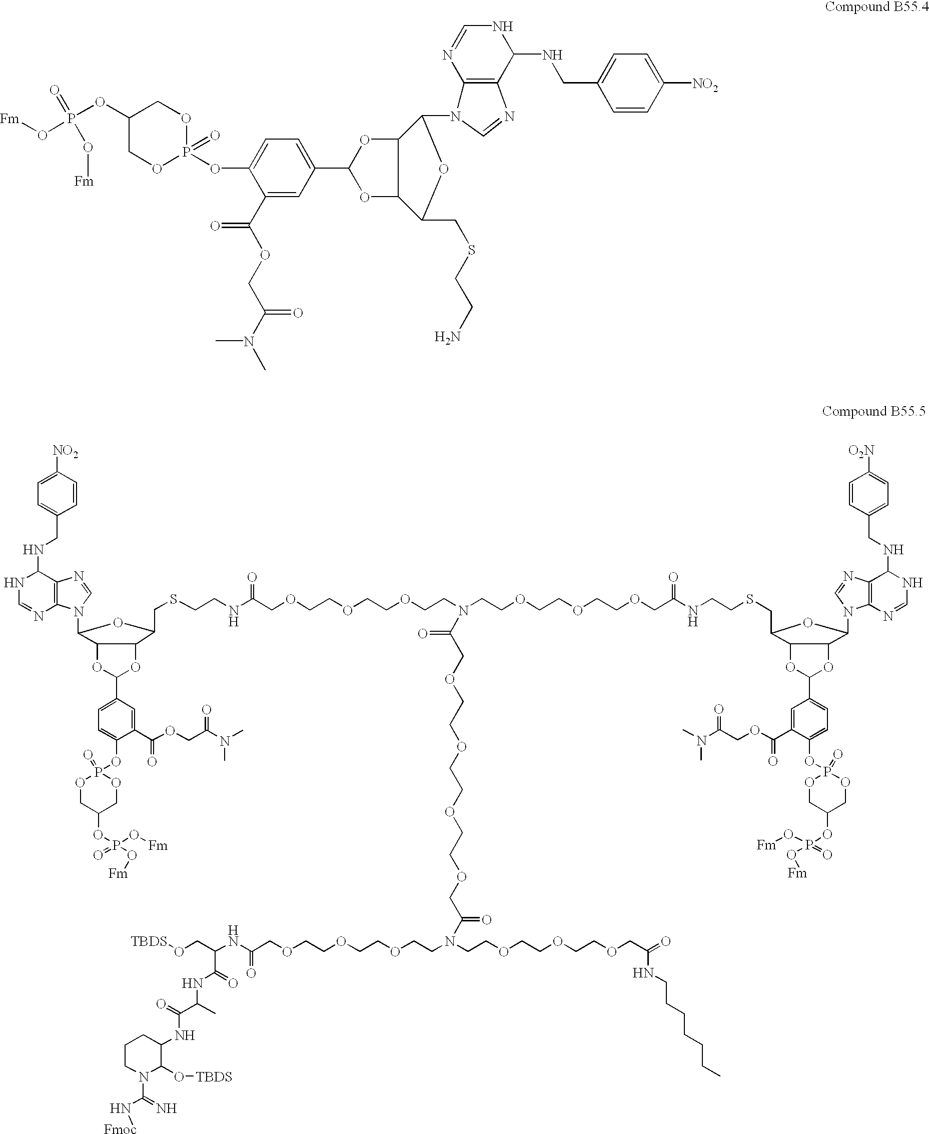

Compound B55.1 may be prepared by a multi-step process. Treating {2-[2-(2-{2-[2-(2-Carboxymethoxy-ethoxy)-ethoxy]-ethylamino}-ethoxy)-ethoxy]-ethoxy}-acetic acid (compound B55.1d) with di-t-butyl pyrocarbonate and in an inert solvent will give [2-(2-{2-[{2-[2-(2-Carboxymethoxy-ethoxy)-ethoxy]-ethyl}-(3,3-dimethyl-butyryl)-amino]-ethoxy}-ethoxy)-ethoxy]-acetic acid. Esterification with allyl alcohol followed by acid treatment to remove the t-Boc group will give {2-[2-(2-{2-[2-(2-Allyloxycarbonyl-methoxy-ethoxy)-ethoxy]-ethylamino}-ethoxy)-ethoxy]-ethoxy}-acetic acid allyl ester (compound B55.1a). This may be coupled to {2-[2-(2-tert-Butoxycarbonylmethoxy-ethoxy)-ethoxy]-ethoxy}-acetic acid and the product treated with acid to remove the t-buyl ester to give compound B55.1b.

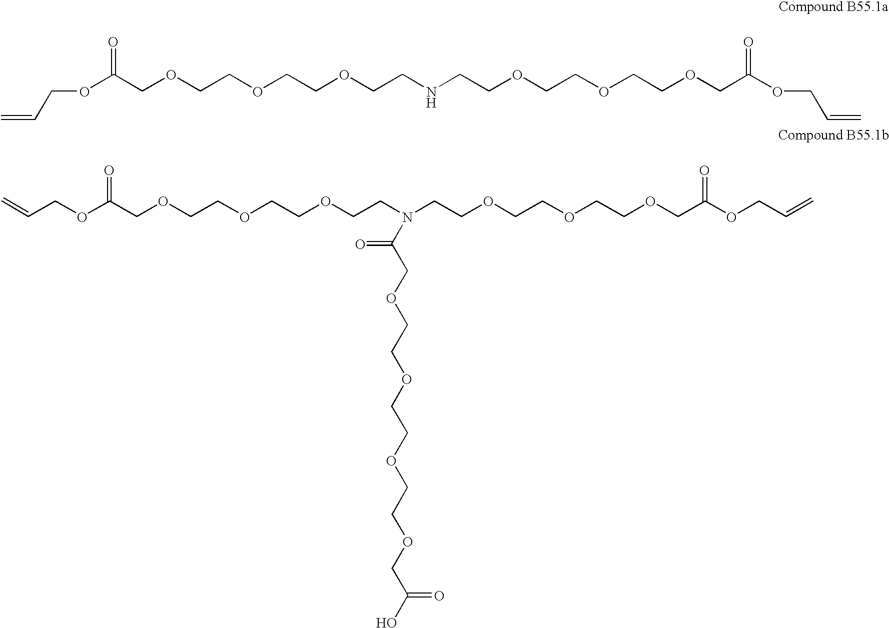

Compound B55.1b may be coupled to compound B55.1c to give compound 55.1.

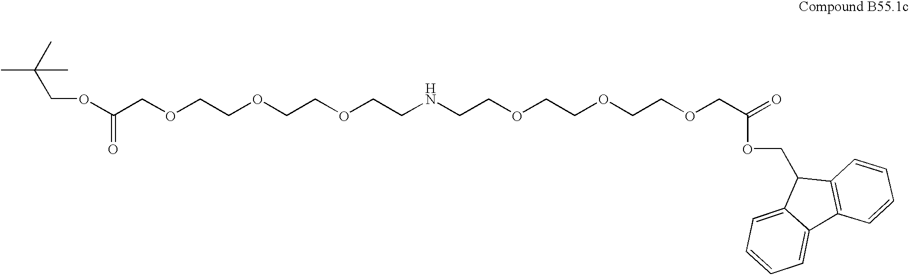

Compound B55.1c may be prepared by a multi-step process. The compound {2-[2-(2-{2-[2-(2-Carboxymethoxy-ethoxy)-ethoxy]-ethylamino}-ethoxy)-ethoxy]-ethoxy}-acetic acid may be treated with 2,2,2 trichloroethyl chloorformate and base in an inert solvent or under Schotten-Bauman conditions. The product may then be esterified with one equivalent of T-butyl alcohol and [2-(2-{2-[(2-{2-[2-(2,2-Dimethyl-propoxycarbonylmethoxy)-ethoxy]-ethoxy}-ethyl)-(2,2,2-trichloroethoxycarbonyl)-amino]-ethoxy}-ethoxy)-ethoxy]-acetic acid isolated. This may then be esterified with (9H-Fluoren-9-yl)-methanol and treated with Zn and acetic acid to give compound B55.1c.

Compound B55.1d may be prepared by reacting {2-[2-(2-Chloro-ethoxy)-ethoxy]-ethoxy}-acetic acid tert-butyl ester and {2-[2-(2-Amino-ethoxy)-ethoxy]-ethoxy}-acetic acid tert-butyl ester in an inert solvent in the presence of base, purifying the product by chromatography and then removing the t-butyl ester groups with acid.

Compound B55.4 may be prepared by a multi-step process. Compound 49.9b may be deprotected with tetrabutylammonium fluoride and treated with Phosphorochloridic acid bis-(9H-fluoren-9-ylmethyl) ester in an inert solvent, and the product treated with an excess of 2,2-dimethoxypropane and acid to give compound B55.4.1. This compound may be reacted with 5′-S-[2-(1,1-dioxobenzo[b]thiophen-2-yloxycarbonylamino)ethyl]-N6-(4-nitrobenzyl)-5′-thioadenosine in presence of an acid, and the product can be selectively deprotected with tris(2-aminoethyl)amine to give compound B55.4.1.5′-S-[2-(1,1-dioxobenzo[b]thiophen-2-yloxycarbonylamino)ethyl]-N6-(4-nitrobenzyl)-5′-thioadenosine can be prepared by reacting of 5′-S-(2-aminoethyl)-N6-(4-nitrobenzyl)-5′-thioadenosine with 1,1-dioxobenzo[b]thiophen-2-yloxycarbony chloride. 5′-S-(2-aminoethyl)-N6-(4-nitrobenzyl)-5′-thioadenosine is a known compound.

Compound B55.2 can be prepared by a method analogous to the method used for compound 14.7

### Example 56

Example 56 is similar to example 55. However, compound A55 is replaced with compound A56 that has a different intracellular trigger.

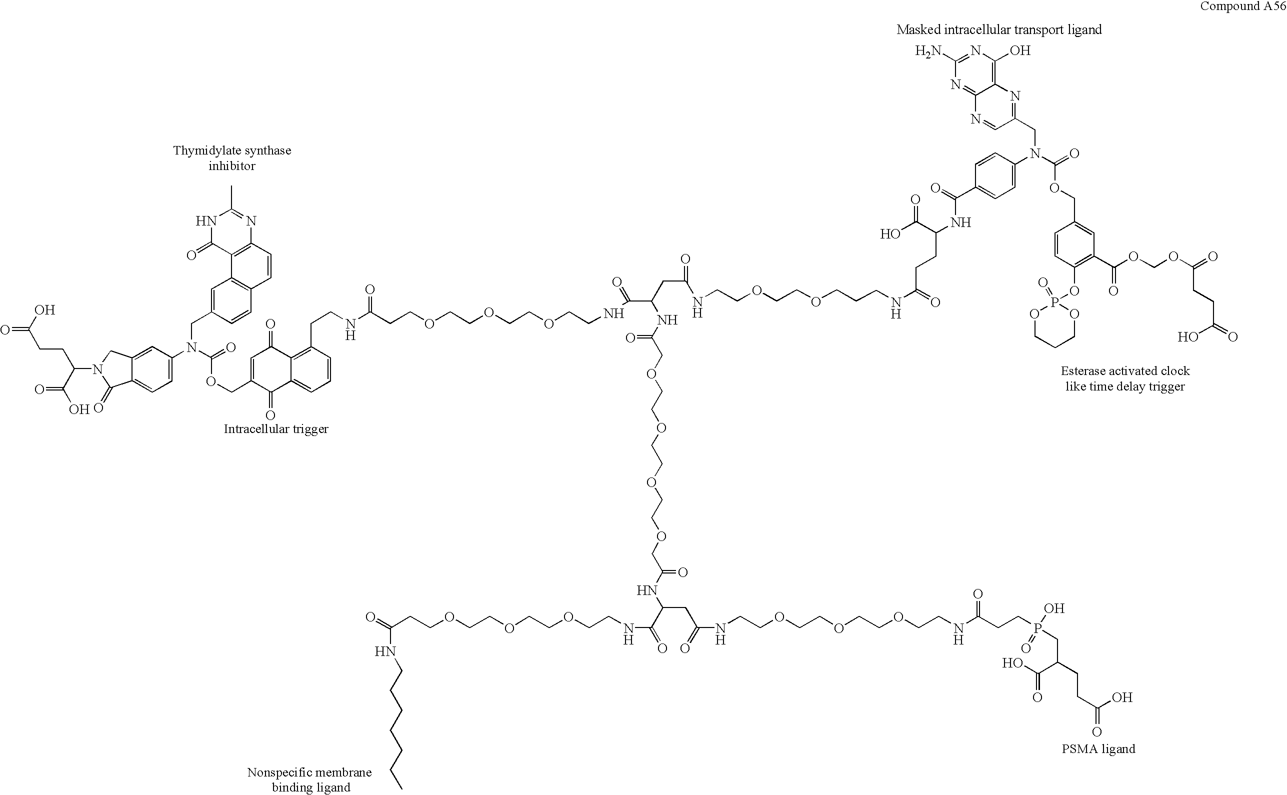

Compound A56 may be prepared by the methods described for compound A55 by replacing compound A55.12 or compound A55.12b with compound A56.1 or compound A56.1.b, respectively.

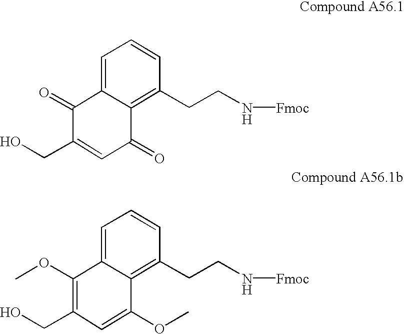

Compound A56.1 may be prepared by a multi-step process. Naphthalene-1,4-diol may be treated with one equivalent of tert-butydimethylchlorosilane and base in an inert solvent to give 4-(tert-butyl-dimethyl-silanyloxy)-naphthalen-1-ol. Treatment with methyl iodide and base will give tert-Butyl-(4-methoxy-naphthalen-1-yloxy)-dimethyl-silane. Heating with hexacarbonylchromium will form the Cr(CO)₃ complex. Treatment with LiCH₂CN in an inert solvent followed by oxidation with iodine will give compound A56.2.

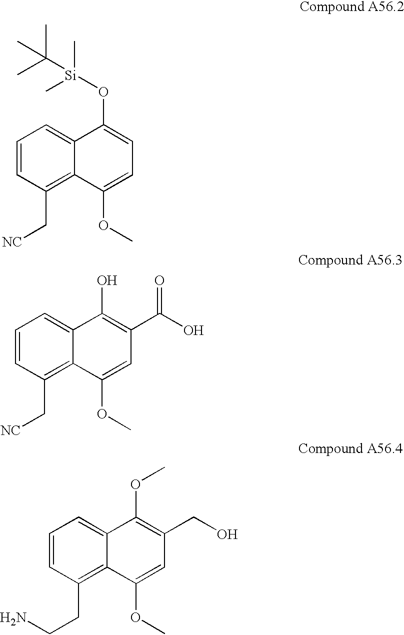

The following references relate to this subject matter: McQuillin F. J., et al., Transition Metal Organometallics for Organic Synthesis, Cambridge University Press, 1991, p.187, the contents of which are incorporated herein by reference in their entirety.

The silyl protecting group may be removed with t-butylammonium fluoride and the product may be treated with carbon dioxide and a base such as sodium hydroxide to give compound A56.3. Treatment with methyl iodide and base followed by reduction with lithium aluminum hydride in an inert solvent will give compound A56.4. Treatment with 9-fluorenylmethyl chloroformate in an inert solvent will give compound A56.1b. Treatment of compound A56.1b with chloroacetic anhydride and base, followed by oxidation with cerium (IV) ammonium nitrate in an inert solvent, followed by hydrolysis of the chloroacetate ester will give compound A56.1.

### Example 57

Example 57 is similar to example 56, however, compound B55 is replaced with compound B57. Compound B57 has two targeting ligands for urokinase and two masked nucleoside transport inhibitors that are based on dipyridamole. The dipyridamole groups are masked with esterase activated clock like time delay triggers. An additional phosphate group on the dipyridamole moiety will be cleaved by phosphatases.

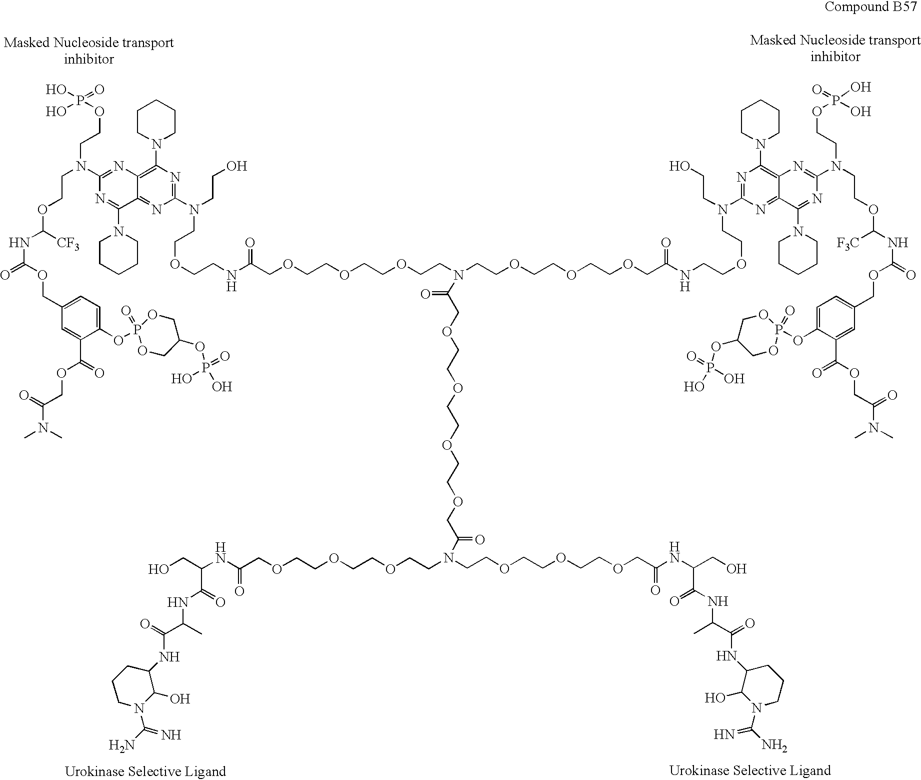

Compound B57 may be prepared by the method described for compound B55 by replacing heptylamine with compound B55.2 and also replacing compound B55.4 with compound B57.1.

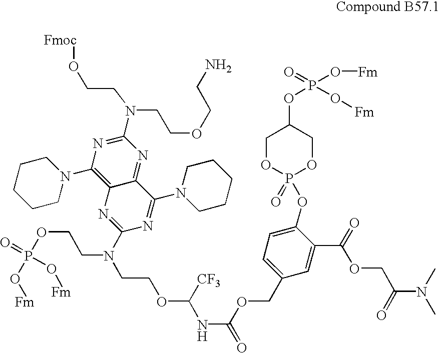

Compound B57.1 may be prepared by reacting compound B57.2 and compound B57.3 in an inert solvent in the presence of base and then cleaving the trichloroethyl ester with Zn and phosphate buffer.

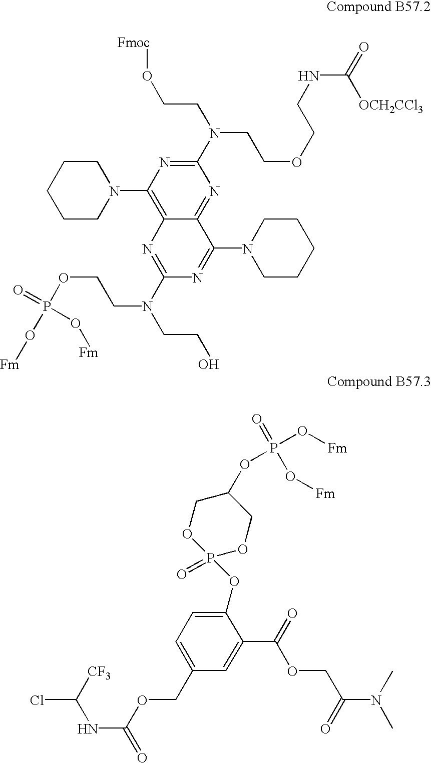

Compound B57.2 may be prepared by a multi-step process. Reacting 2,6-Dichloro-4,8-di-piperidin-1-yl-pyrimido[5,4-d]pyrimidine with (2-{2-[2-(Tetrahydropyran-2-yloxy)-ethylamino]-ethoxy}-ethyl)-carbamic acid 2,2,2-trichloro-ethyl ester in an inert solvent in the presence of base will give compound B57.4. Reacting compound B57.4 with carbonic acid tert-butyl ester 2-[2-(tert-butyl-dimethyl-silanyloxy)-ethylamino]-ethyl ester in an inert solvent in the presence of a base will give compound B57.5.

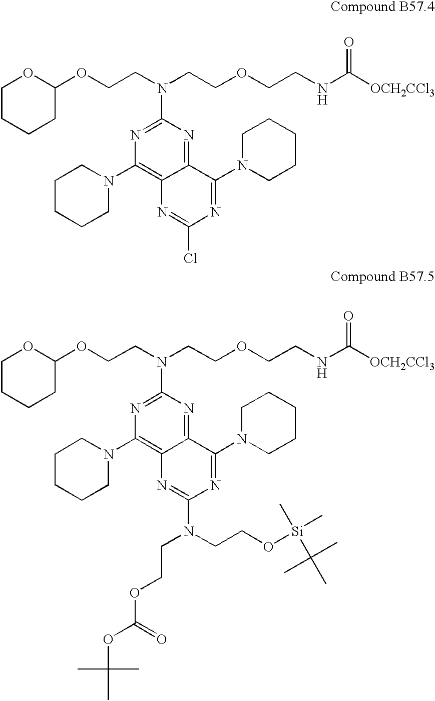

Compound B57.5 may be treated with tetrabutylammonium fluoride in an inert solvent to remove the silyl protecting group. The product may then be reacted with phosphorochloridic acid bis-(9H-fluoren-9-ylmethyl) ester and a base such as triethylamine in an inert solvent. The product may then be treated with acid to selectively remove the tetrahydropyranyl protecting group. The product my then be reacted with 9H-fluoren-9-ylmethyl chloroformate in the presence of a base such as pyridine in an inert solvent. The product may then be treated with acid to remove the t-Boc group and give compound B57.2.

The compound (2-{2-[2-(Tetrahydro-pyran-2-yloxy)-ethylamino]-ethoxy}-ethyl)-carbamic acid 2,2,2-trichloro-ethyl ester may be prepared by a multi-step process. Reacting 2-[2-(2-Amino-ethoxy)-ethylamino]-ethanol with carbonic acid 2,5-dioxo-pyrrolidin-1-yl ester 2,2,2-trichloro-ethyl ester will give {2-[2-(2-Hydroxy-ethylamino)-ethoxy]-ethyl}-carbamic acid 2,2,2-trichloro-ethyl ester. Treatment with one equivalent benzyl chloroformate and pyridine will give (2-Hydroxy-ethyl)-{2-[2-(2,2,2-trichloro-ethoxycarbonylamino)-ethoxy]-ethyl}-carbamic acid benzyl ester. Treatment with acid catalyst and dihydropyran will give [2-(Tetrahydro-pyran-2-yloxy)-ethyl]-{2-[2-(2,2,2-trichloroethoxycarbonylamino)-ethoxy]-ethyl}-carbamic acid benzyl ester. Catalytic hydrogenation with Pd on carbon will give the desired final product.

The compound carbonic acid tert-butyl ester 2-[2-(tert-butyl-dimethyl-silanyloxy)-ethylamino]-ethyl ester may be prepared by a multi-step process. Treating 2-(2-Hydroxy-ethylamino)-ethanol with benzyl chloroformate and a base such as pyridine in an inert solvent will give bis-(2-hydroxy-ethyl)-carbamic acid benzyl ester. Treatment with 1 equivalent of tert-butyldimethylchlorosilane and base in an inert solvent will give, after purification, [2-(tert-Butyl-dimethyl-silanyloxy)-ethyl]-(2-hydroxy-ethyl)-carbamic acid benzyl ester. Treatment with di-t-butyl pyrocarbonate and base in an inert solvent followed by catalytic hydrogenation with Pd on carbon will give the desired final product.

Compound B57.3 may be prepared by a multi-step process. Treating compound 49.9b with acid or a reagent such as tetrabutylammonium fluoride will remove the silyl protecting group. The product may then be reacted with phosphorochloridic acid bis-(9H-fluoren-9-ylmethyl) ester and a base such as triethylamine in an inert solvent. The aldehyde group may then be reduced by catalytic hydrogenation with Pd on carbon or with a reagent such as sodium borohyd ride. The product may then be treated with phosgene in an inert solvent. The product may then be treated with one equivalent of ammonia and a base in an inert solvent. The product may then be treated with trifluoroacetaldehyde. The product may then be treated with a reagent such as phosphorous trichloride to give compound B57.3.

### Example 58

Example 58 is similar to example 57, however, a different nucleoside transport inhibitor is employed. In compound B58, an analog of dilazep is employed as the nucleoside transport inhibitor. The dilazep analog is masked with an esterase activated clock like time delayed trigger. In example B58, the amide analog of dilazep is employed. The following references relate to this subject matter: Gati W. P.; Paterson A. R. P., “Interaction of [³H]Dilazep at Nucleoside Transporter-Associated Binding Sites on S49 Mouse Lymphoma Cells,” Molecular Pharmacology, 3:134-141(1989), the contents of which are incorporated herein by reference in their entirety.

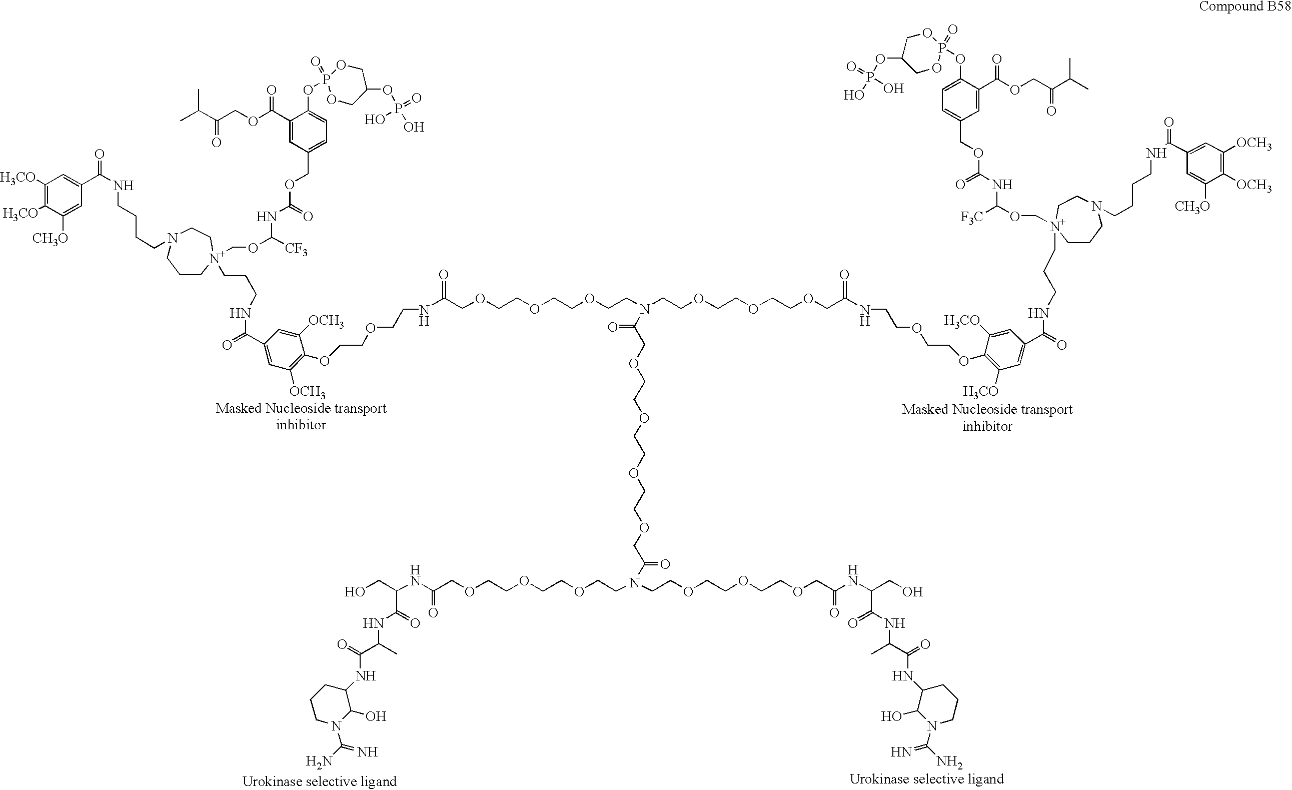

Compound B58 may be employed by the methods described for compound B57 by replacing compound B57.1 with compound B58.1.

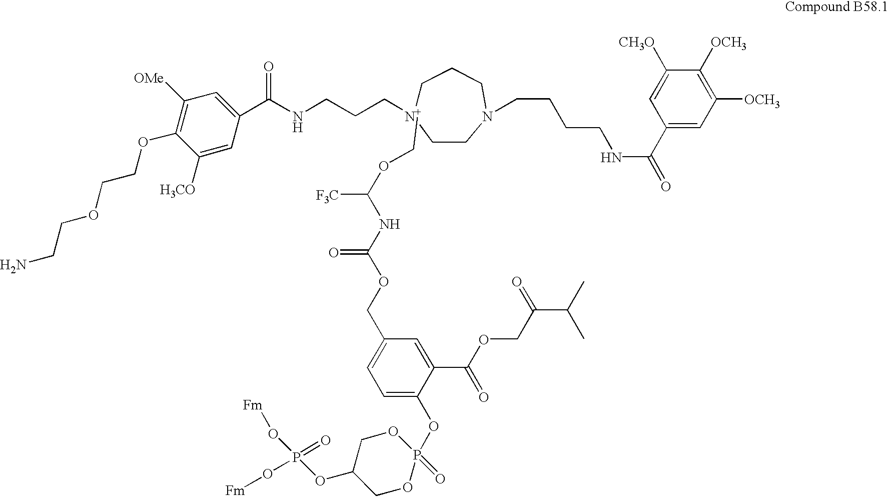

Compound B58.1 may be prepared by a mult-step process. Treating 1,4-diazacycloheptane with toluene-4-sulfonic acid 3-tert-butoxycarbonylaminopropyl ester and base in an inert solvent followed by treatment with acid to remove the t-Boc group will give 3-[1,4]Diazepan-1-yl-propylamine. Treatment with trifluoroacetic anhydride and base will give 2,2,2-Trifluoro-N-{3-[4-(2,2,2-trifluoro-acetyl)-[1,4]diazepan-1-yl]-propyl}-acetamide, compound B58.2. Treatment of compound B58.3 with iodochloromethane and a base such as N,N-diisoproplyethylamine will give the corresponding chloromethyl derivative, which can be reacted with compound B58.2 to give compound B58.4.

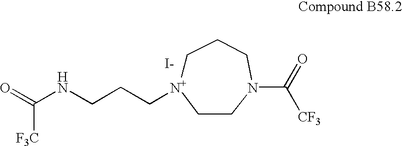

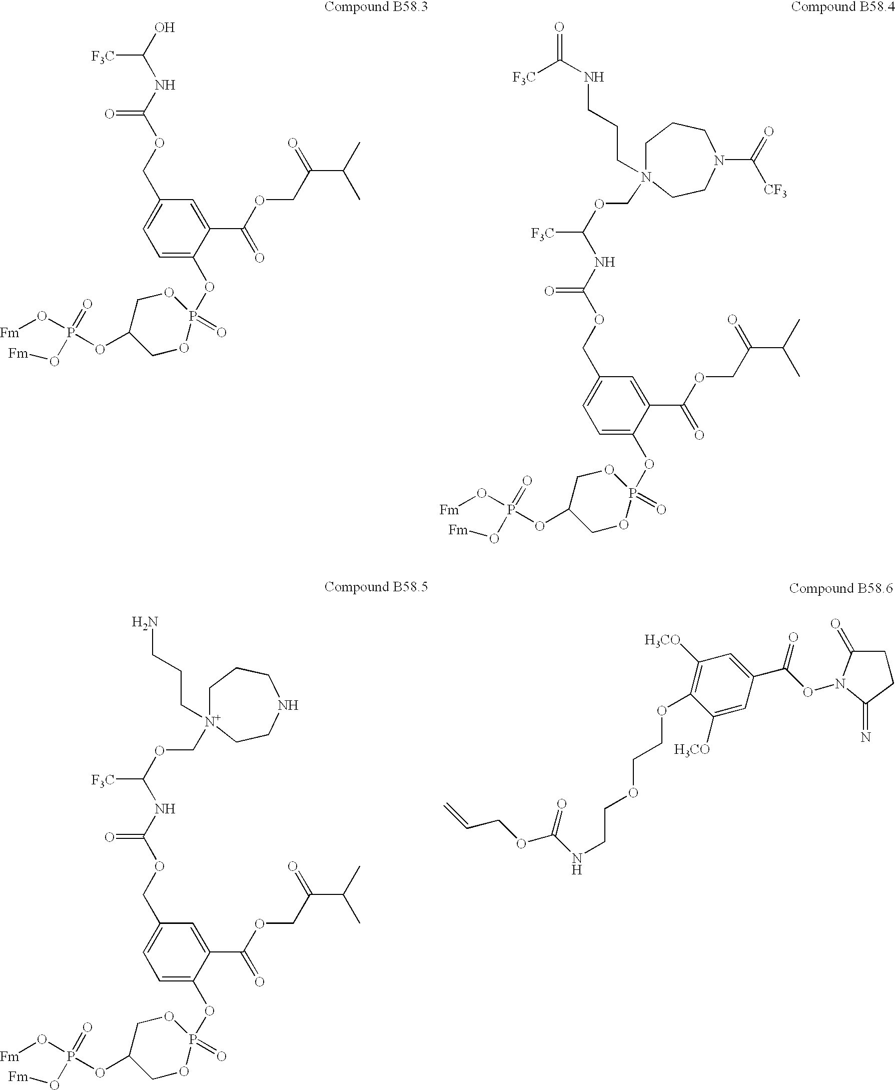

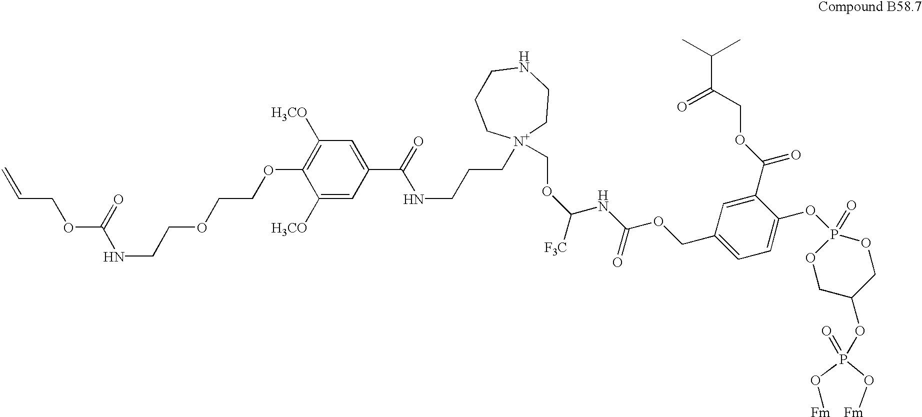

Selective removal of the trifluoroacetyl groups with a reagent, such as tris(2-aminoethyl)amine and a transesterification catalyst, such as distannoxane will give compound B58.5. Reacting with compound B58.6 in an inert solvent will give compound B58.7. Reacting compound B58.7 with 3,4,5-trimethoxy-N-(3-oxo-propyl)-benzamide in an inert solvent, in the presence of acid catalyst with removal of water, followed by reduction with a reagent such as sodium cyanoborohydride will give B58.8.

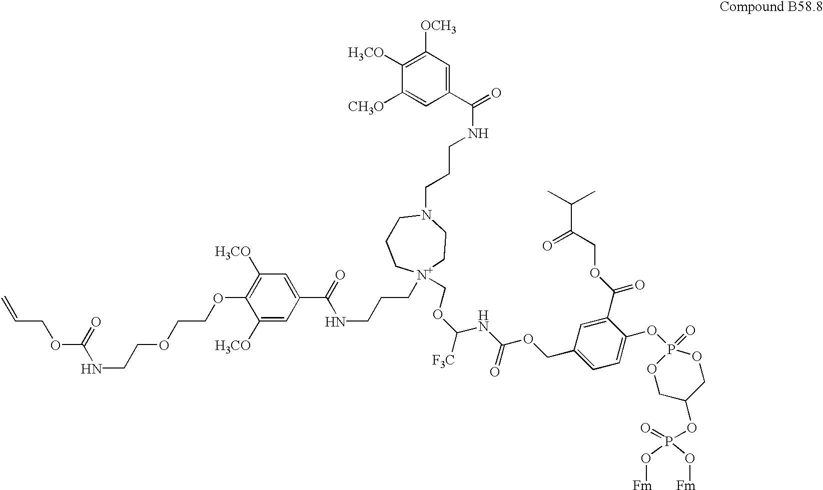

Removal of the alloxycarbonyl protecting group with Pd(0) will give compound B58.1.

Compound B58.6 may be prepared by a multi-step process. Treating 2-(2-Amino-ethoxy)-ethanol with di-t-butyl pyrocarbonate and in an inert solvent will g i v e [2-(2-Hydroxy-ethoxy)-ethyl]-carbamic acid tert-butyl ester. Treating with tosyl chloride and base in an inert solvent will give Toluene-4-sulfonic acid 2-(2-tert-butoxycarbonylamino-ethoxy)-ethyl ester. Reacting with 4-Hydroxy-3,5-dimethoxy-benzoic acid tert-butyl ester and a strong base will give 4-[2-(2-tert-Butoxycarbonyl-amino-ethoxy)-ethoxy]-3,5-dimethoxy-benzoic acid tert-butyl ester. Treatment with acid will give 4-[2-(2-Amino-ethoxy)-ethoxy]-3,5-dimethoxy-benzoic acid. Treatment with allyl chloroformate under Schotten-Bauman conditions followed by coupling to N-hydroxysuccinimide with a reagent such as dicyclohexylcarbodiimide will give compound B58.6.

### Example 59

Compounds A59, B55, C59 and folic acid are a set of compounds, which when used in combination will exhibit multifactorial targeting with synergistic toxicity against tumor cells that jointly express urokinase, MMP2, 3, 9,12, and 13, and laminin receptors.

Compound A59 will deliver trimetrexate to laminin receptor positive cells. Trimetrexate is a potent inhibitor of dihydrofolate reductase.

Compound B55 will deliver to urokinase positive cells masked “5′-S-(2-Aminoethyl)-N6-(4-Nitrobenzyl)-5′-Thioadenosine ligands, which when unmasked will bind tightly to nucleoside transport proteins on the surface of the targeted cells.

Compound C59 will deliver AG2034 to MMP2, 3,9,12, and 13 positive cells. AG2034 is a potent inhibitor of glycinamide ribonucleotide formyltransferase.

Pronounced synergistic toxicity is expected in cells that are jointly targeted by compounds A59, B55, and C59 in the presence of exogenous folate. The following references relate to this subject matter: Gaumont Y., et al., “Quantitation of Folic Acid Enhancement of Antifolate Synergism,” Cancer Res, 52:2228-2235 (1992); Faessel H. M., et al., “Super in Vitro Synergy between Inhibitors of Dihydrofolate Reductase and Inhibitors of Other Folate-requiring Enzymes: The Critical Role of Polyglutamylation,” Cancer Res, 58:3036-3050 (1998), the contents of which are incorporated herein by reference in their entirety.

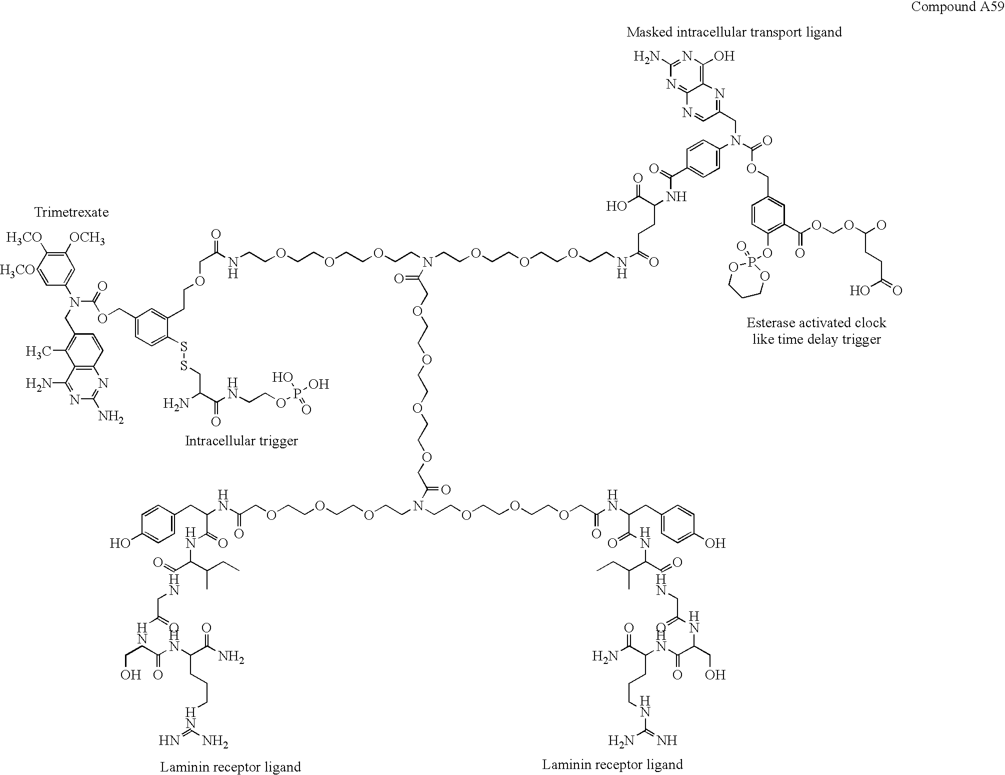

Compound A59 may be prepared by a mult-step procedure. Compound A59.1 may be coupled with two equivalents of A59.2. Treatment with tris(2-aminoethyl)amine under conditions that will leave the Fmoc group intact will cleave the Bsm ester. The product may then be coupled to compound A59.3. Treatment with dilute acid followed by base will then give compond A59.

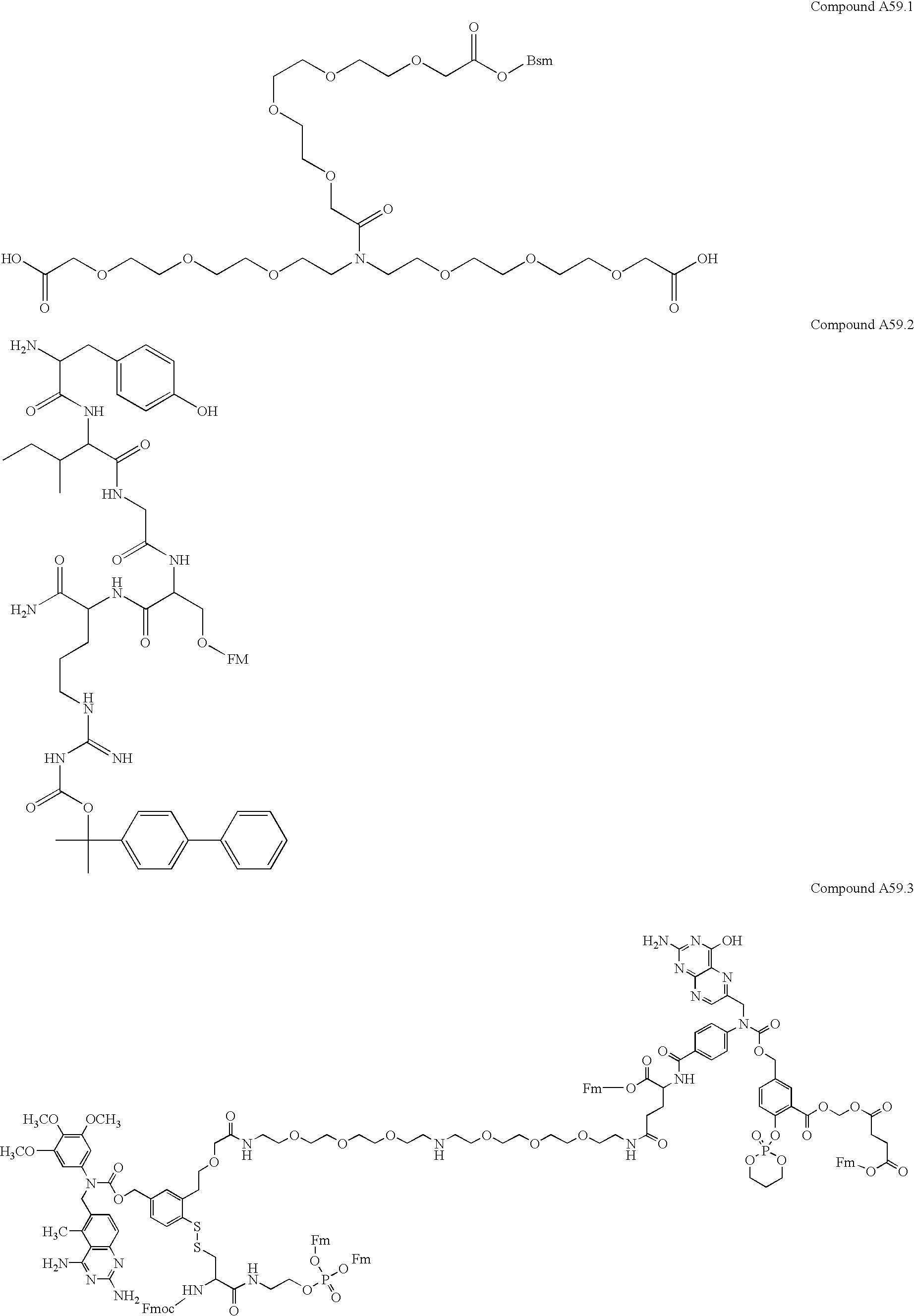

Compound A59.1 may be prepared by coupling compound B55.1a and (2-{2-[2-(1,1-Dioxo-1H-116-benzo[b]thiophen-2-ylmethoxycarbonylmethoxy)-ethoxy]-ethoxy}-ethoxy)-acetic acid and then cleaving the allyl esters with Pd(0).

Compound A59.2 may be prepared by routine methods of oligopeptide synthesis.

Compound A59.3 may be prepared by coupling compound A59.4 and A59.5, treating with Zn and acid to remove the trichloroethoxycarbonyl protecting group, coupling with A59.6, and then treating with tris(2-aminoethyl)amine under conditions that will leave the Fmoc group intact to remove Bsmoc group.

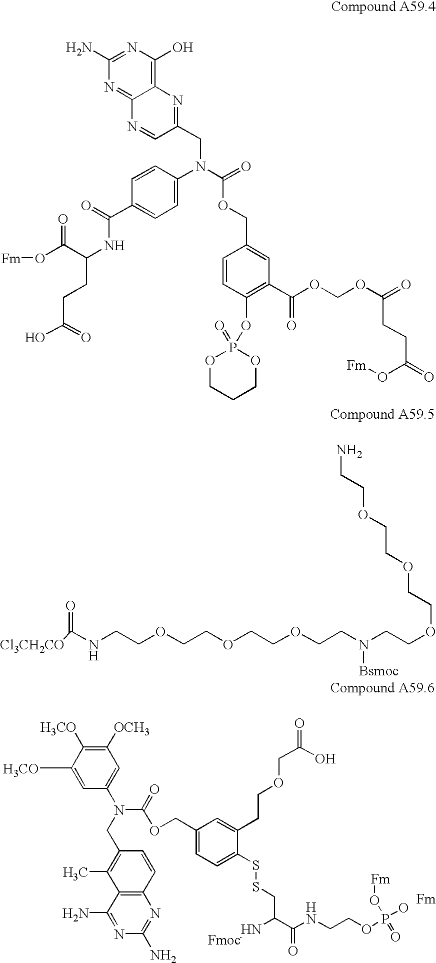

The synthesis of compound A59.4 has been given elsewhere. Compound A59.5 may be prepared by a multi-step procedure. Treating Bis-(2-{2-[2-(2-amino-ethoxy)-ethoxy]-ethoxy}-ethyl)-amine with one equivalent of trityl chloride and base in an inert solvent will give, after purification, (2-{2-[2-(2-Amino-ethoxy)-ethoxy]-ethoxy}-ethyl)-[2-(2-{2-[2-(trityl-amino)-ethoxy]-ethoxy}-ethoxy)-ethyl]-amine. Treatment with 1 equivalent of carbonic acid 2,5-dioxo-pyrrolidin-1-yl ester 2,2,2-trichloro-ethyl ester will give {2-[2-(2-{2-[2-(2-{2-[2-(Trityl-amino)-ethoxy]-ethoxy}-ethoxy)-ethylamino]-ethoxy}-ethoxy)-ethoxy]-ethyl}-carbamic acid 2,2,2-trichloro-ethyl ester. Treatment with 1 equivalent of (1,1-Dioxo-1H-1λ6-benzo[b]thiophen-2-yl)-methyl chloroformate and base, followed by HCl treatment to remove the trityl group will give compound A59.5.

Compound A59.6 may be prepared by reacting trimetrexate and compound 23.2b in an inert solvent in the presence of a base such as pyridine and then treating with tris(2-aminoethyl)amine under conditions that will leave the Fmoc group intact to cleave the Bsm ester.

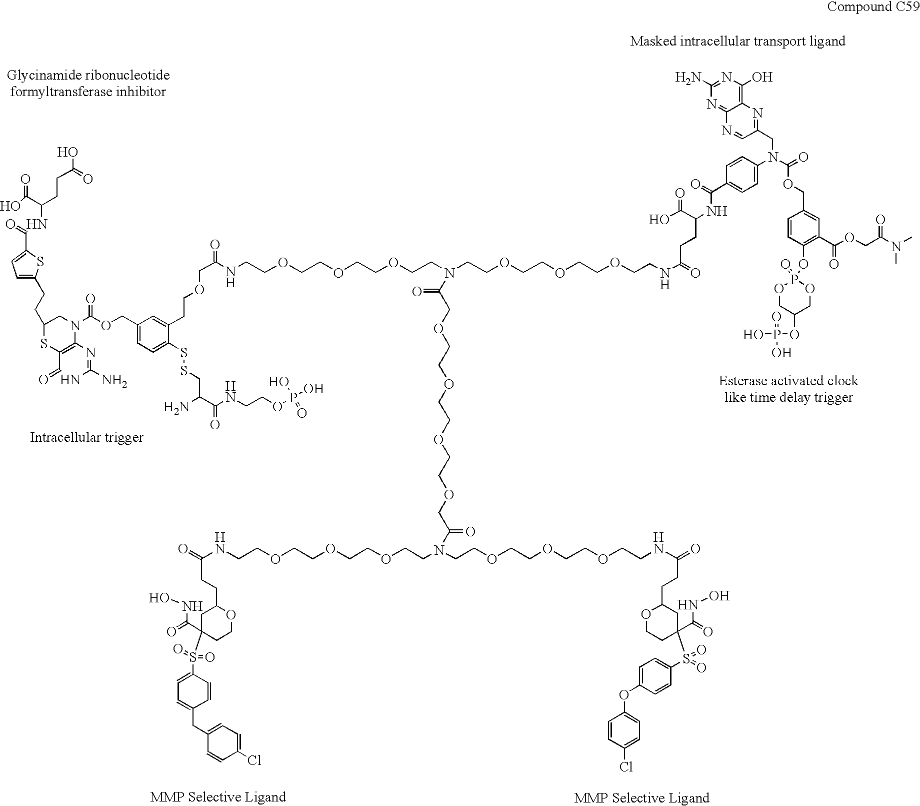

Compound C59 may be prepared by a multistep procedure. Compound C59.1 may be coupled with two equivalents of compound C59.2.

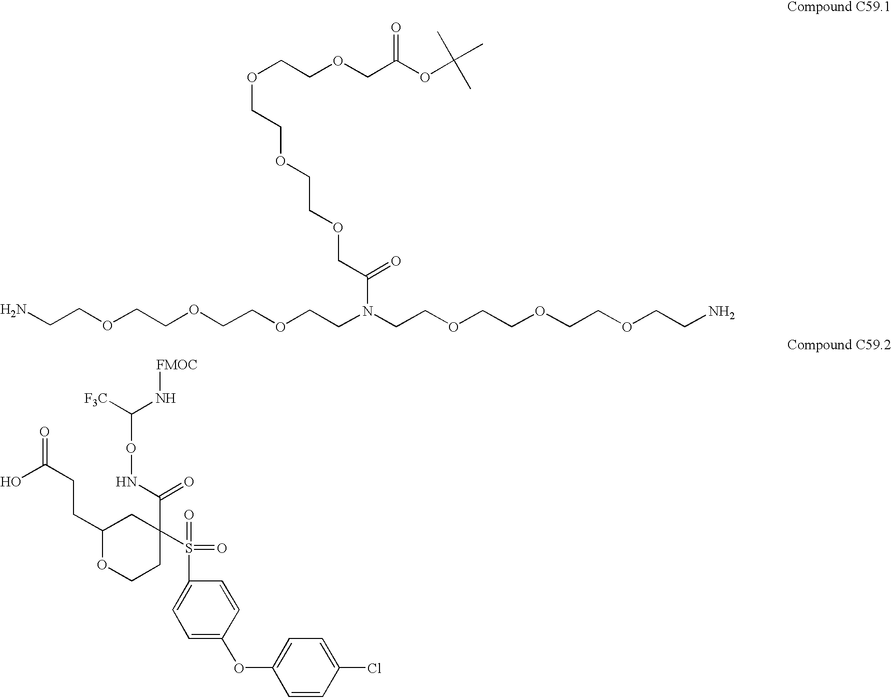

Treatment with trifluoracetic acid will remove the t-butyl ester. The product may then be coupled to compound C59.3. Treatment with base will then give compound C59.

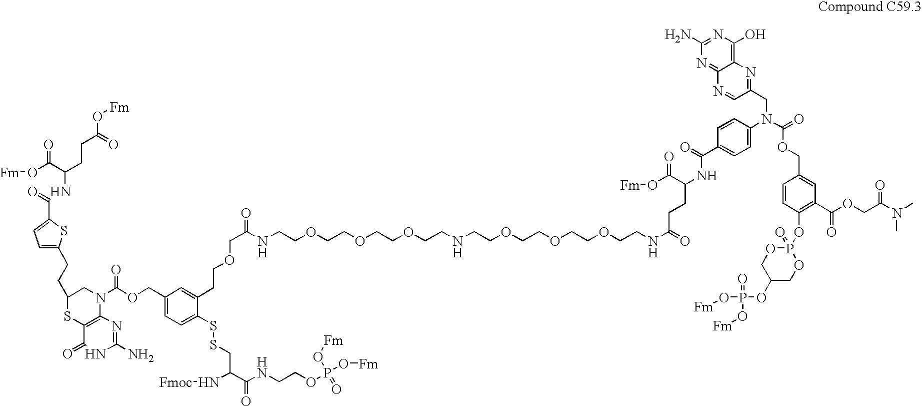

Compound C59.1 may be prepared by a multi-step process. The compound 2-(2-{2-[2-(Trityl-amino)-ethoxy]-ethoxy}-ethoxy)-ethylamine may be coupled to {2-[2-(2-Benzyloxycarbonylamino-ethoxy)-ethoxy]-ethoxy}-acetic acid. The product may then be reduced with a reagent such as lithium aluminum hydride in an inert solvent. The product may then be treated with trityl chloride and base in an inert solvent to give Bis-[2-(2-{2-[2-(trityl-amino)-ethoxy]-ethoxy}-ethoxy)-ethyl]-amine. This may then be coupled to {2-[2-(2-tert-Butoxycarbonylmethoxy-ethoxy)-ethoxy]-ethoxy}-acetic acid and treated with dilute acid to remove the trityl groups to give compound C59.1.

The synthesis of compound C59.2 was given in example 17.

Compound C59.3 may be prepared by a multi-step procedure. Compound C59.4 may be coupled to compound C59.5. Treatment with Pd(0) will remove the allyloxycarbonyl protecting group. The product may then be coupled with compound C59.6. Treatment with tris(2-aminoethyl)amine under conditions that will leave the Fmoc group intact will give compound C59.3.

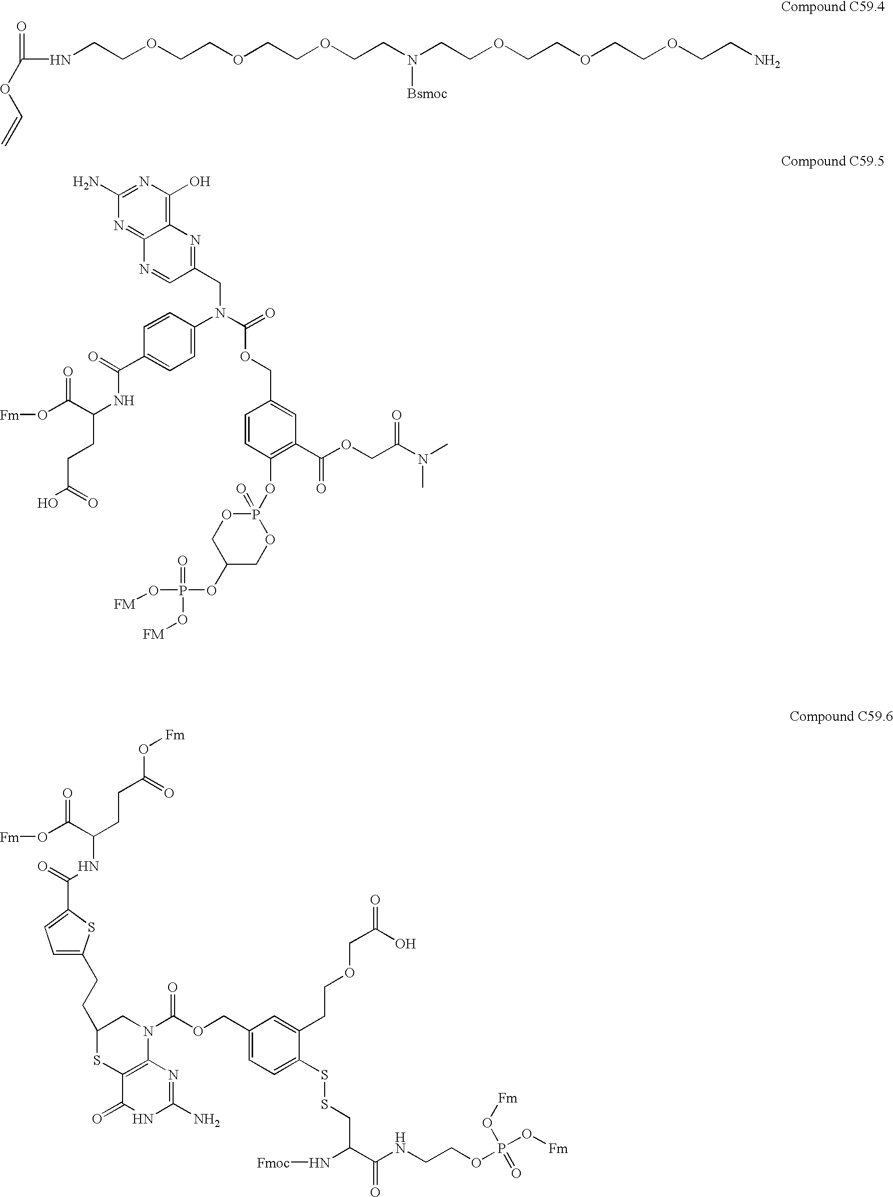

Compound C59.4 may be prepared by a multi-step process. Treating Bis-(2-{2-[2-(2-amino-ethoxy)-ethoxy]-ethoxy}-ethyl)-amine with 1 equivalent of trityl chloride and isolating the monosubstituted product will give (2-{2-[2-(2-Amino-ethoxy)-ethoxy]-ethoxy}-ethyl)-[2-(2-{2-[2-(trityl-amino)-ethoxy]-ethoxy}-ethoxy)-ethyl]-amine. Treating with one equivalent of a reagent such as carbonic acid allyl ester 2,5-dioxo-pyrrolidin-1-yl ester in an inert solvent, followed with (1,1-Dioxo-1H-1λ6-benzo[b]thiophen-2-yl)-methyl chloroformate and base, followed by HCL treatment to remove the trityl group will give compound C59.4 as the hydrochloride salt.

Compound C59.5 may be prepared by reacting C59.5a and C59.5b in an inert solvent in the presence of a base such as pyridine and then treating with Zn and acid to remove the trichloroethyl group.

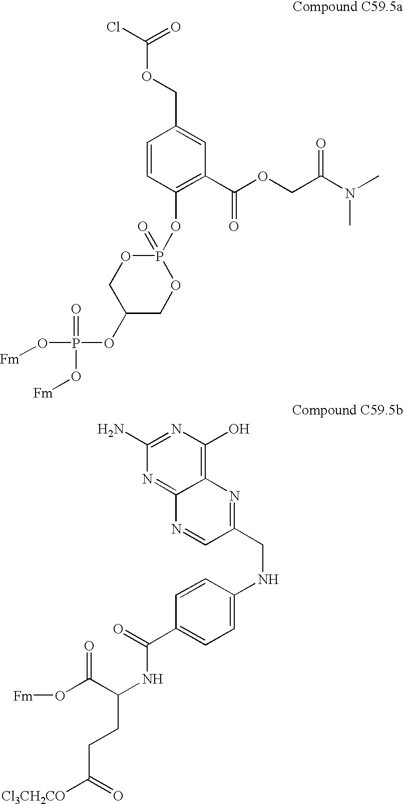

Compound C59.6 may be prepared by coupling reacting compound C59.6a and compound C59.6b in an inert solvent in the presence of a base followed by removal of the silyl and Bsm protecting groups. The silyl based protecting group may be removed by a reagent such as pyridine HF. The Bsm ester may be selectively cleaved with tris(2-aminoethyl)amine under conditions that will leave the Fmoc group intact.

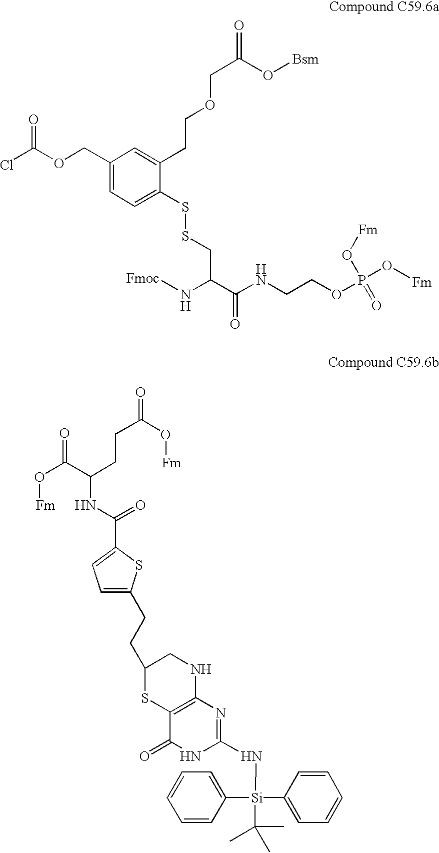

Compound C59.6b may be prepared by treating compound C59.6c with tert-butylchlorodiphenylsilane in an inert solvent in the presence of base. Compound C59.6c may be prepared by coupling compound C59.6d and L-glutamic acid di-(9H-Fluoren-9-yl)-methyl ester. Compound C59.6d is a known compound.

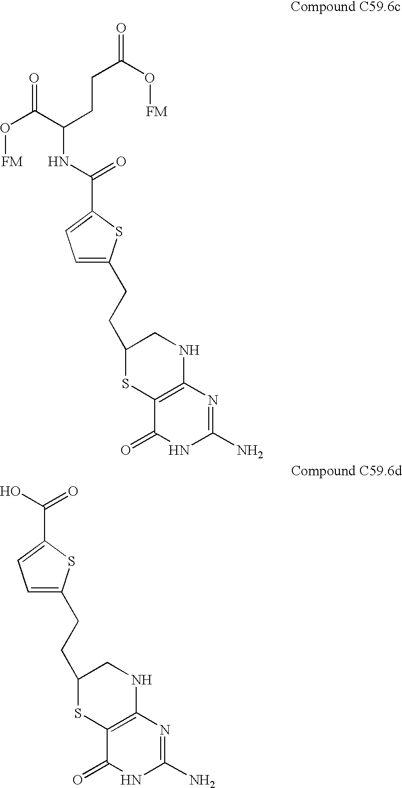

The following references relate to this subject matter: Varney M. D., et al., “Protein Structure-Based Design, Synthesis, and Biological Evaluation of 5-Thia-2,6-diamino-4(3H)-oxopyrimidines: Potent Inhibitors of Glycinamide Ribonucleotide Transformylase with Potent Cell Growth Inhibition,” J Med Chem, 40:2502-2524 (1997); Overman L. E., et al., “tert-Butyldiphenylsilylamines: A Useful Protecting Group for Primary Amines,” Tetrahedron Let, 27(37):4391-4394 (1986), the contents of which are incorporated herein by reference in their entirety.

### Example 60

Compounds A59, B57, C59 and folic acid are a set of compounds which when used in combination will exhibit multifactorial targeting against tumor cells that jointly express urokinase, MMP2, 3, 9, 12, and 13, and laminin receptors.

### Example 61.1

Compounds A59, B58, C59 and folic acid are a set of compounds, which when used in combination, will exhibit multifactorial targeting against tumor cells that jointly express urokinase, MMP2, 3, 9,12, and 13, and laminin receptors.

### Example 61.2

Compounds A61, B57, and C61 are a set of compounds, which when used in combination, will exhibit targeting against tumor cells that jointly express urokinase, and PSMA or MMP(2, 3, 9, 12, or 13) and PSMA. Compound A61 will deliver mycophenolic acid, a potent inhibitor of inosine monophosphate dehydrogenase, to PSMA positive cells. Compound B57 will deliver a nucleoside transport inhibitor based on dipyridamole to the surface of urokinase positive cells.

Compound C61 will deliver an ImmucillinGP analog to MMP 2, 3, 9, 12 or 13 positive cells. ImmucillinGP is a potent inhibitor of hypoxanthine-guanine phosphoribosyltransferase. Jointly targeted cells will be exposed to inhibitors of both the denovo and salvage pathways of guanine nucleotide synthesis.

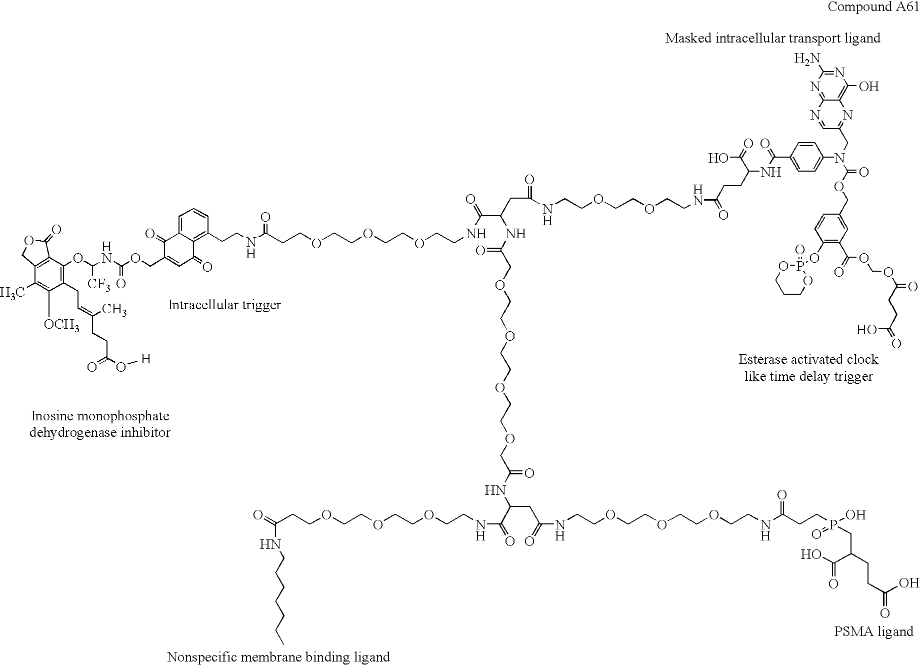

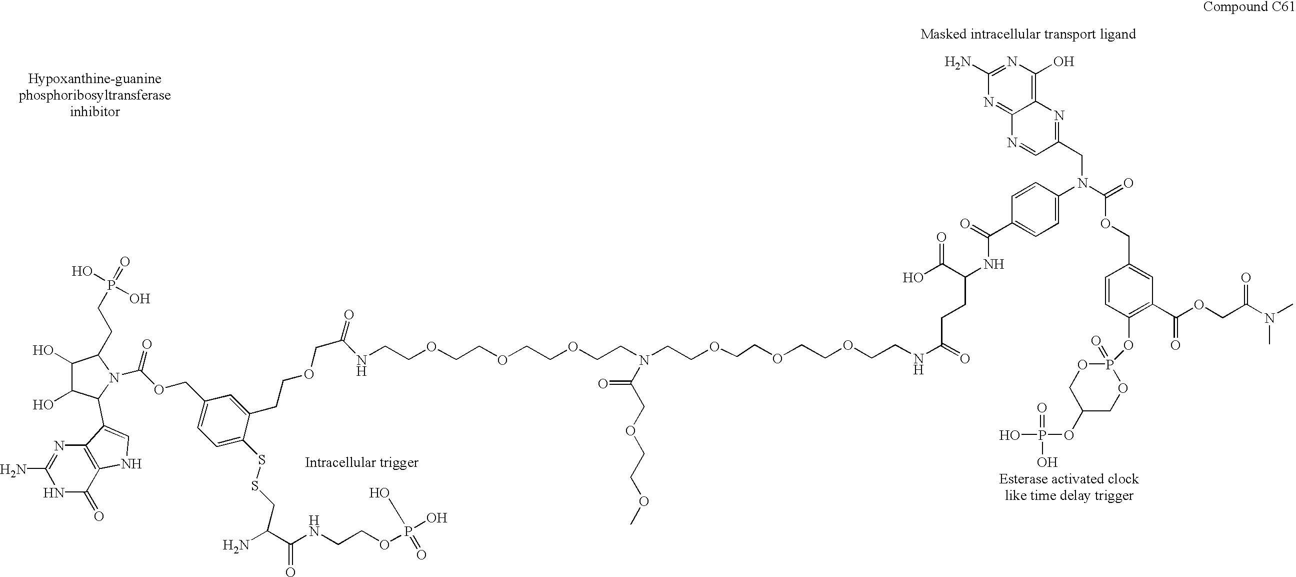

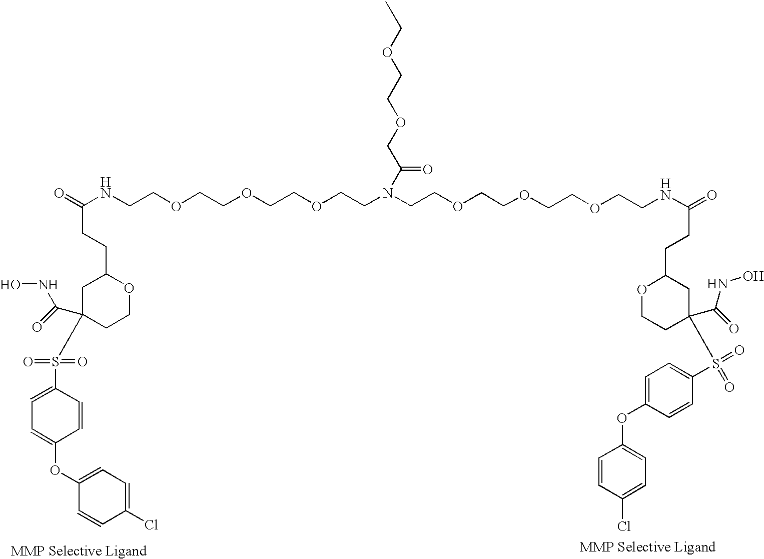

Compound A61 may be prepared by coupling compound A55.5 and compound A61.1a.

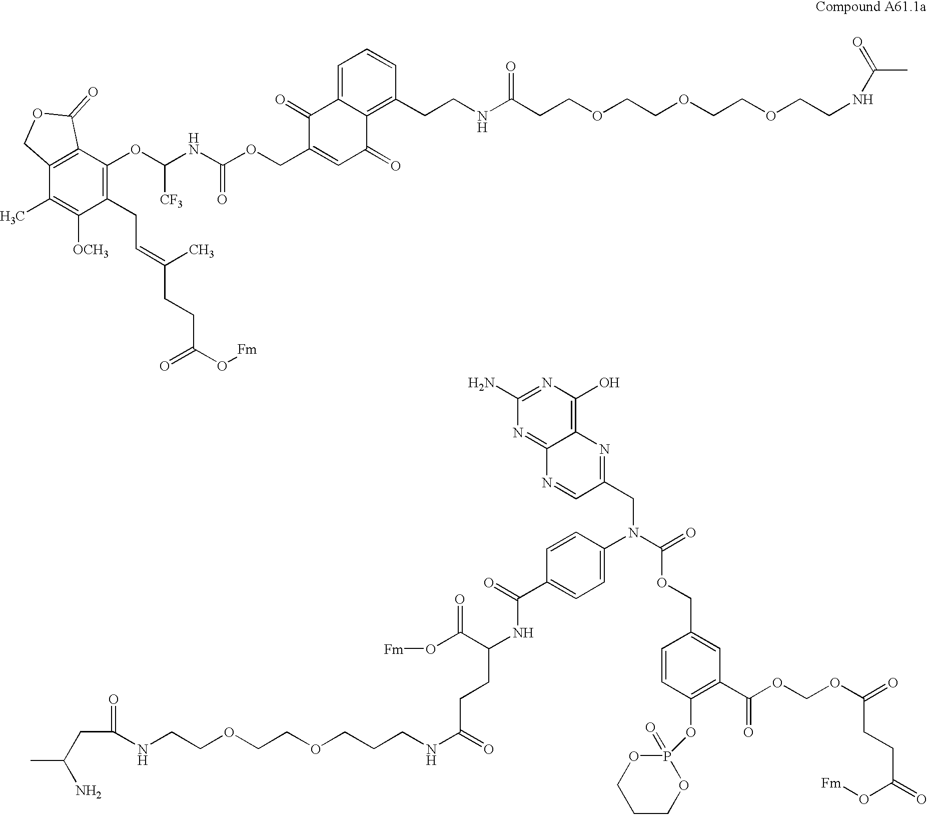

Compound A61.1a may be prepared by coupling compound A61.1b and compound A61.1c and then treating with acid to remove the 2-Biphenyl-4-yl-propan-2-oxy-carbonyl protecting group.

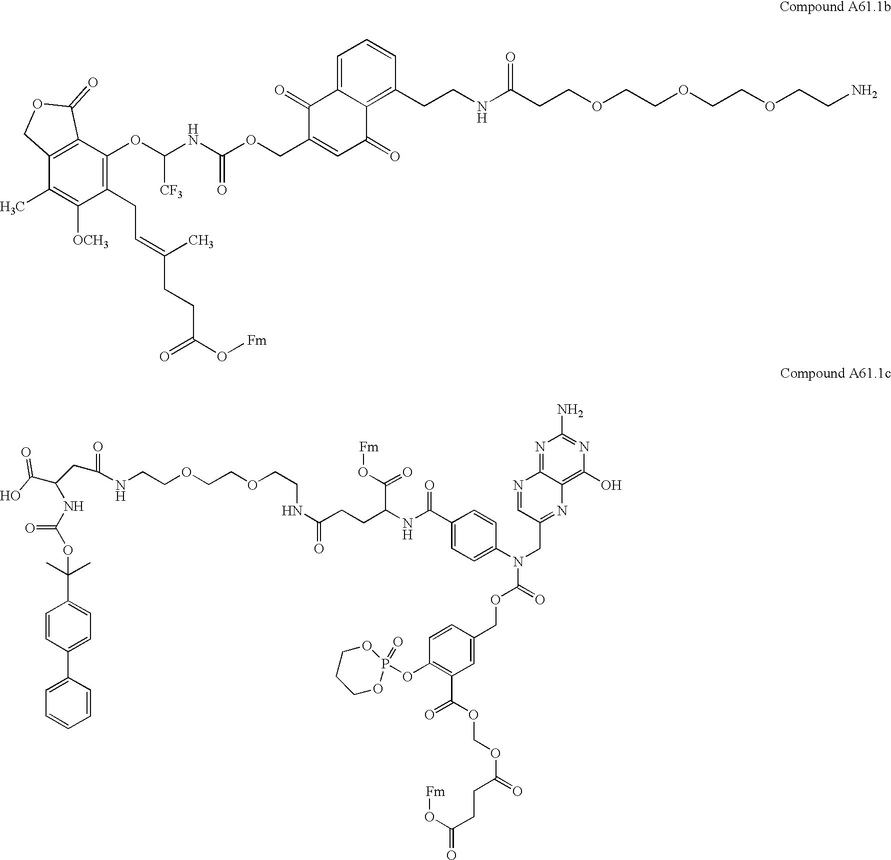

Compound A61.1b may be prepared by reacting compound A61.2 and compound A61.3 in the presence of base in an inert solvent and then treating with acid to remove t-Boc group.

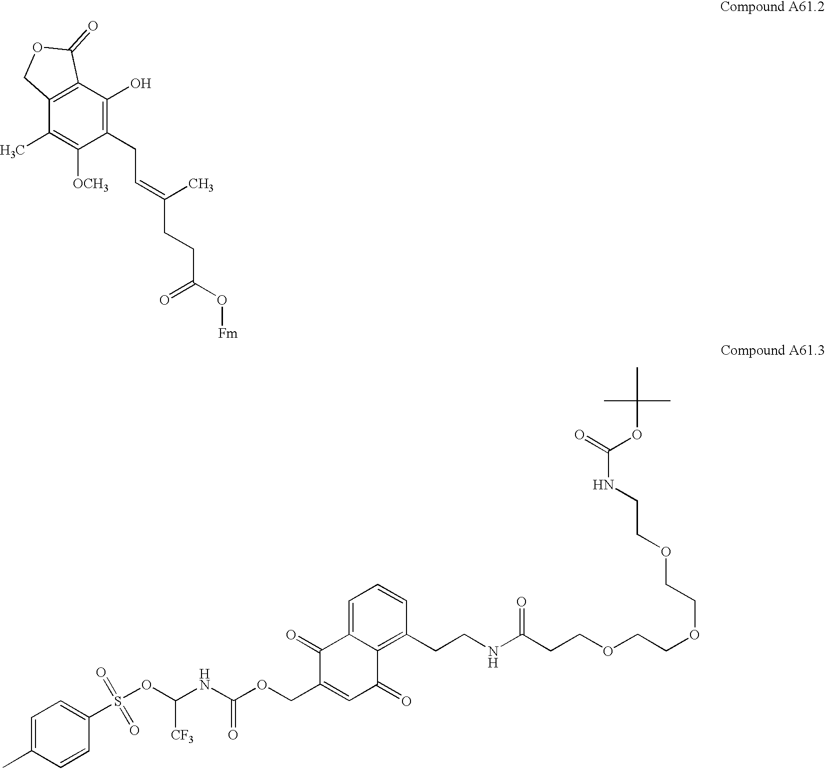

Compound A61.2 may be prepared by treating mycophenolic acid with (9H-Fluoren-9-yl)-methanol and an agent such as dicyclohexylcarbodiimide in an inert solvent. Alternatively the phenol hydroxyl may be protected before esterification.

Compound A61.3 may be prepared by a multi-step process. Compound A56.4 may be coupled with 3-{2-[2-(2-tert-Butoxycarbonyl amino-ethoxy)-ethoxy]-ethoxy}-propionic acid in an inert solvent. The product may then be treated with phosgene and a base such as pyridine, followed by ammonia at low temperature to give compound A61.4.

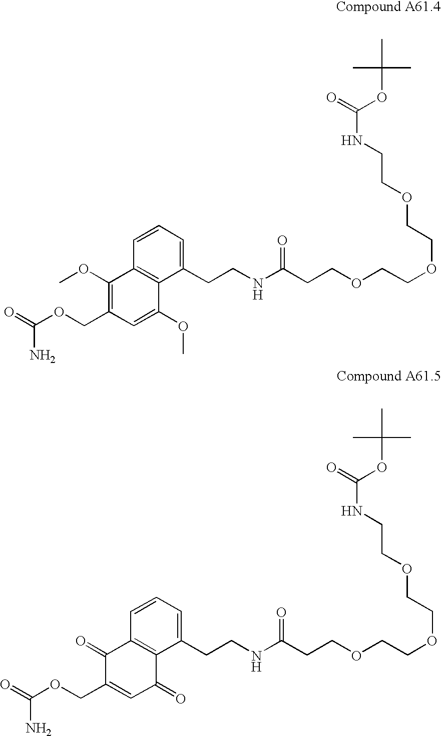

Compound A61.4 may be treated with cerium (IV) ammonium nitrate in an inert solvent to give compound A61.5. Treatment of compound A61.5 with trifluoroacetaldehyde followed by tosyl chloride and base in an inert solvent will give compound A61.3.

Compound A61.1c may be prepared by treating compound 32.1 with N-hydroxysuccinimide and dicyclohexylcarbodiimde to form the active ester, and then reacting the product with compound A61.6 in an inert solvent in the presence of base.

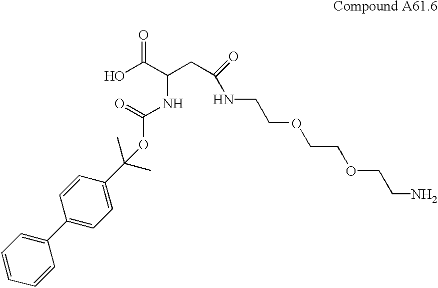

Compound A61.6 (as the salt) may be prepared by coupling L-N-(2-Biphenyl-4-yl-propan-2-oxy-carbonyl) aspartic acid a methyl ester with 2-[2-(2-Amino-ethoxy)-ethoxy]-ethylamine and then cleaving the methyl ester with base.

Compound C61 may be prepared by the method described for compound C59 by replacing compound C59.6b with compound 20.9.

### Example 62

Compounds A61, B57, and C62 are a set of compounds, which when used in combination, will exhibit targeting against tumor cells that jointly express urokinase, and PSMA or MMP(2, 3, 9,12, or 13) and PSMA. Compound C62 is similar to compound C61 except that lmmucillinGP, rather than the phosphonate analog of lmmucillinGP, is employed. Also, a different intracellular trigger is employed.

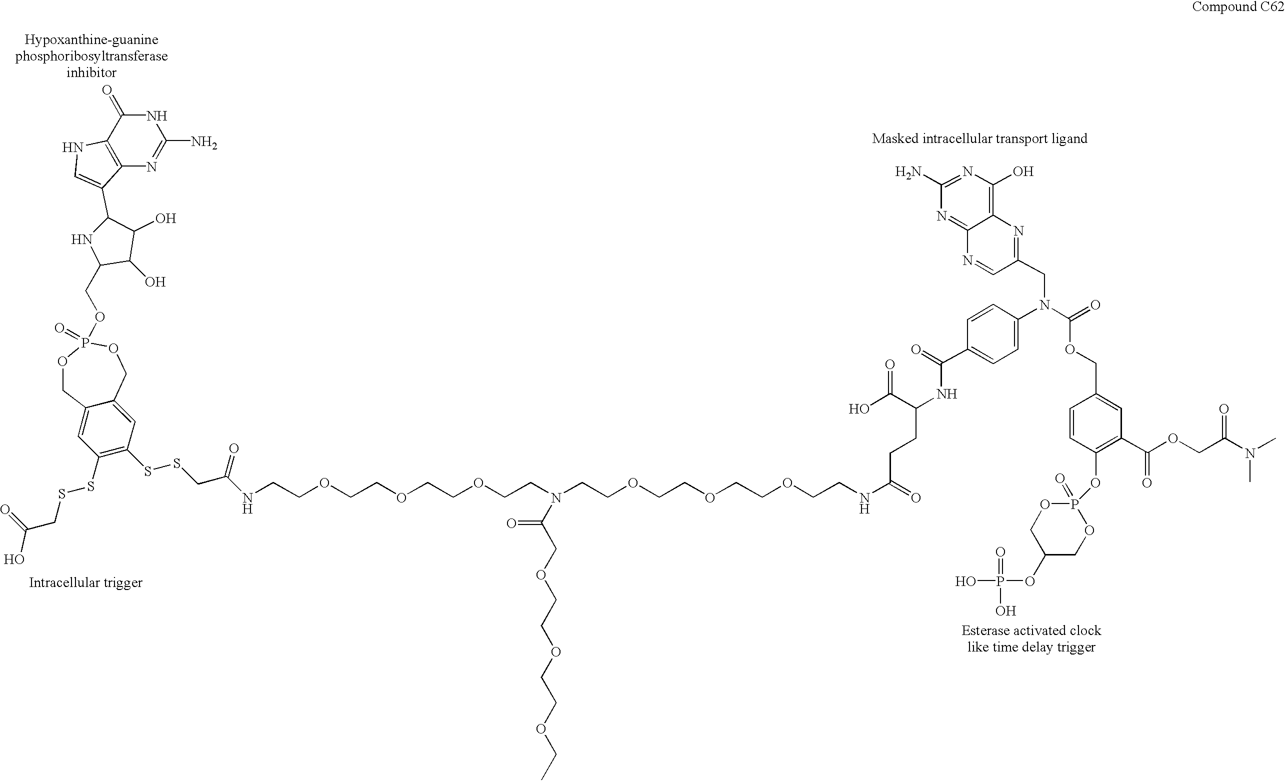

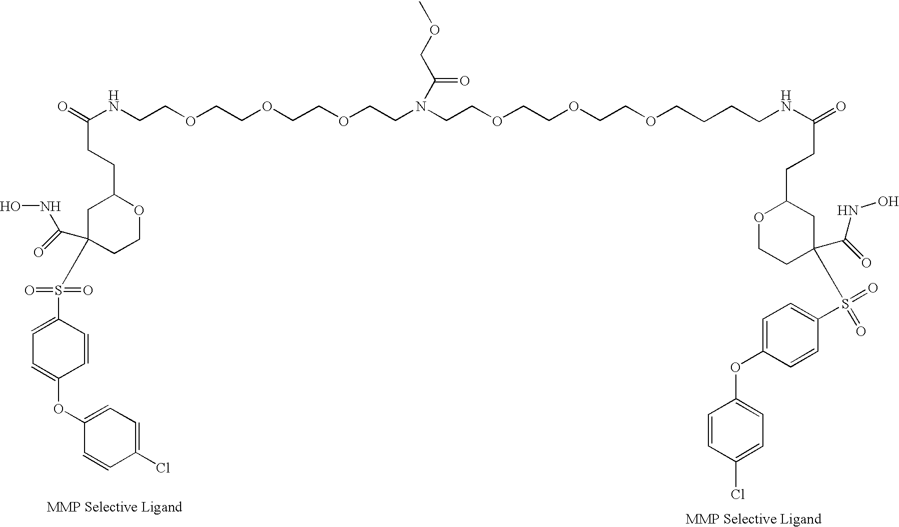

Compound C62 may be prepared by the method described for compound C59 by replacing compound C59.6 with compound C62.1.

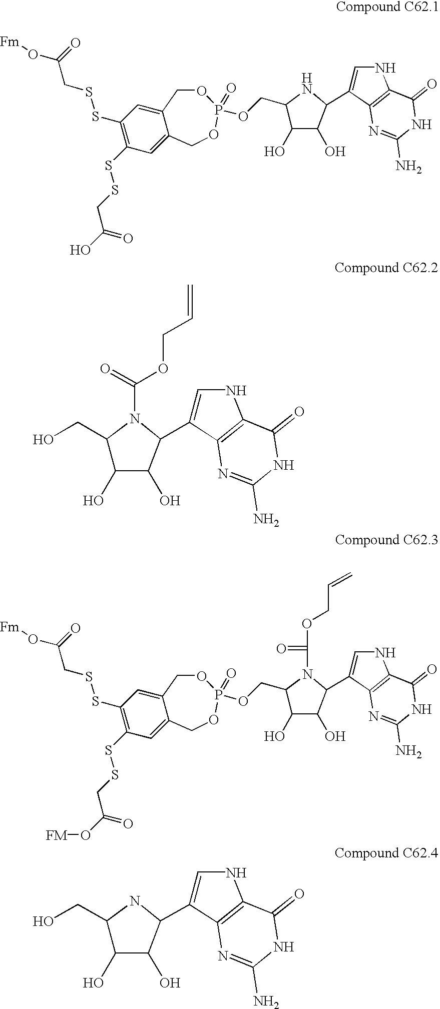

Compound C62.1 may be prepared by reacting compound C62.2 and compound 42.2 in an inert solvent in the presence of base, to give compound C62.3.

Treatment with one equivalent of strong base, followed by removal of the allyloxycarbonyl protecting group with Pd(0), will give compound C62.1.

Compound C62.2 may be prepared by treating compound C62.4 with one equivalent of allyl chloroformate and base in an inert solvent.

### Example 63

Compound A63 and compound B55 are a set of compounds that will selectively target cells that are positive for both urokinase and PSMA. Compound A63 will deliver brequinar, a potent inhibitor of dihydroorotic acid dehydrogenase to PSMA positive cells. Dihydroorotic acid dehydrogenase is the fourth enzyme in the committed pathway of de novo pyrimidine synthesis. Compound B55 will deliver to urokinase positive cells a nucleoside transport inhibitor.

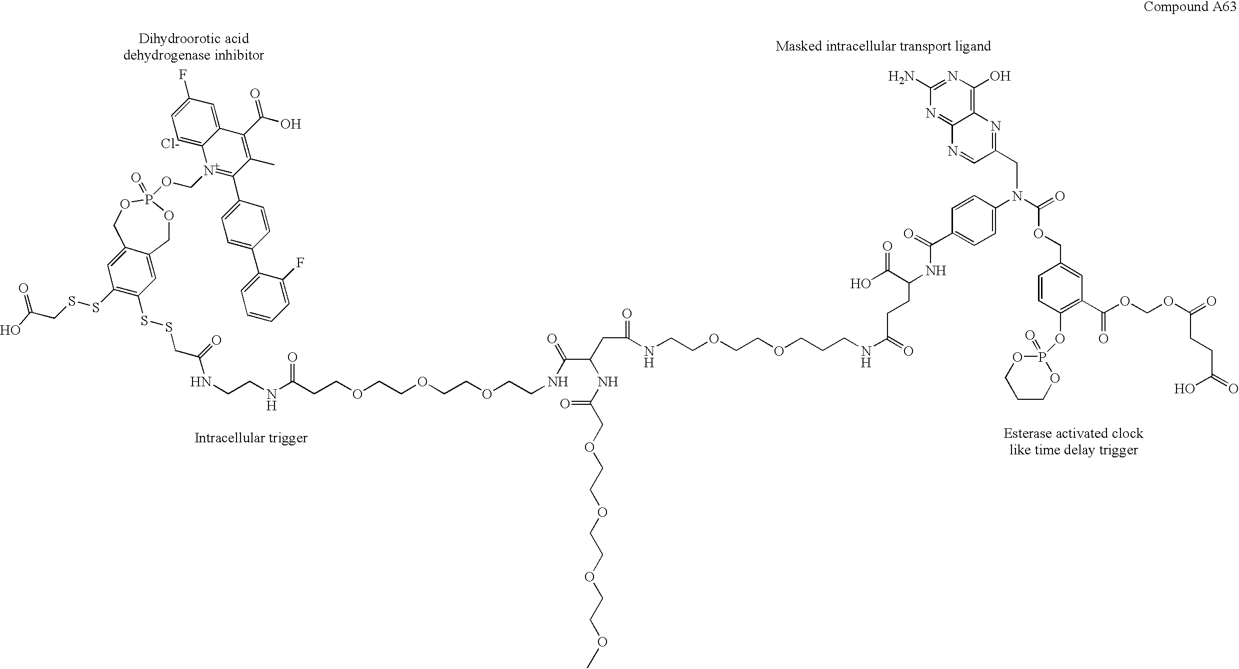

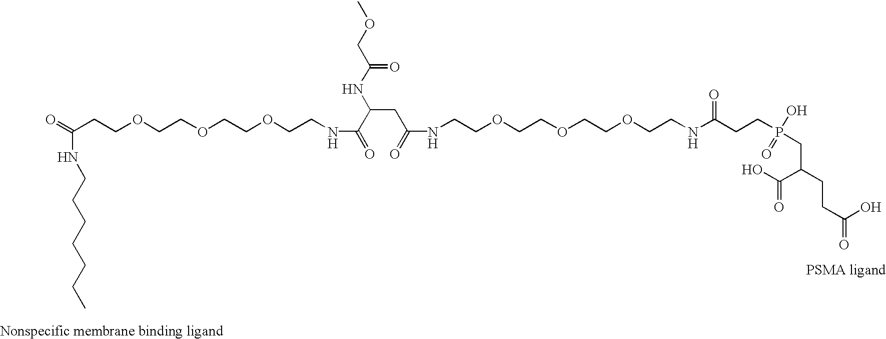

Compound A63 may be prepared by the method described for compound A55 by replacing compound A55.9 with compound A63.1.

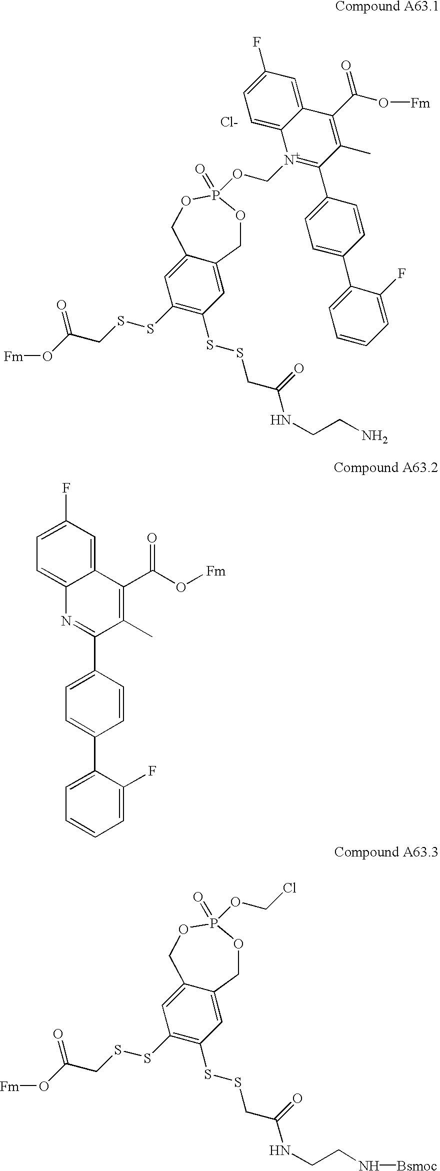

Compound A63.1 may be prepared by a multi-step process. Reacting A63.2 and A63.3 in an inert solvent followed by removal of the Bsm group with tris(2-aminoethyl)amine under conditions that will leave the Fmoc group intact will give compound A63.1.

Compound A63.2 may be prepared by reacting brequinar with (9H-Fluoren-9-yl)-methanol and dicyclohexylcarbodiimide in an inert solvent.

Compound A63.3 may be prepared by a multi-step process. Compound 42.3 may be treated with one equivalent of a strong base to give compound A63.4. This may then be coupled to (2-Amino-ethyl)-carbamic acid 1,1-dioxo-1H-1λ6-benzo[b]thiophen-2-ylmethyl ester to give compound A63.5. Treatment with phosphorous oxychloride and base will give compound A63.6. Treatment with one equivalent of tetrabutylammonium hydroxide in an inert solvent at low temperature will give compound A63.7. Treatment with iodochloromethane and base in an inert solvent will give A63.3.

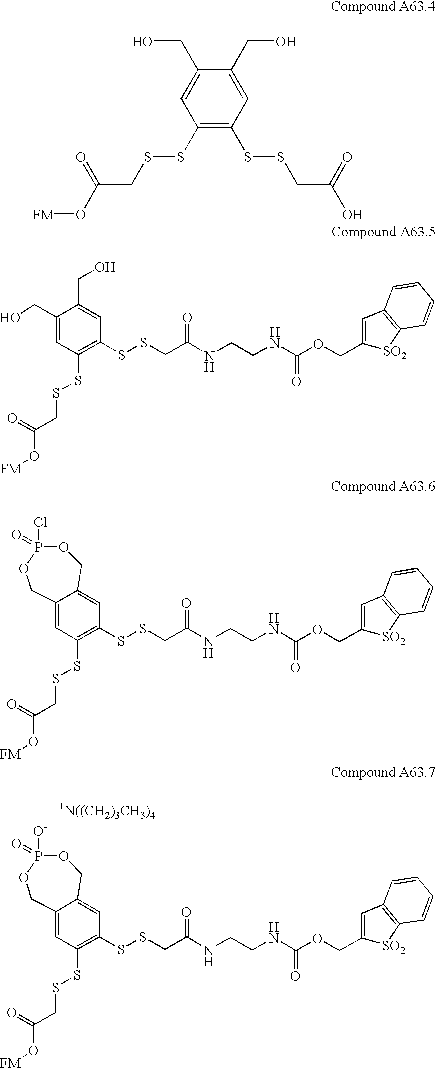

### Example 64

Compound A64 and compound B55 are a set of compounds that will target cells that express MMP (2, 3, 9, 12, or 13) and urokinase. Compound A65 will deliver to MMP+cells a potent inhibitor to Orotidine 5′-phosphate decarboxylase. This enzyme catalyzes the final step in the de novo synthesis of uridine monophosphate. Compound B55 will deliver to urokinase positive cells a nucleoside transport inhibitor.

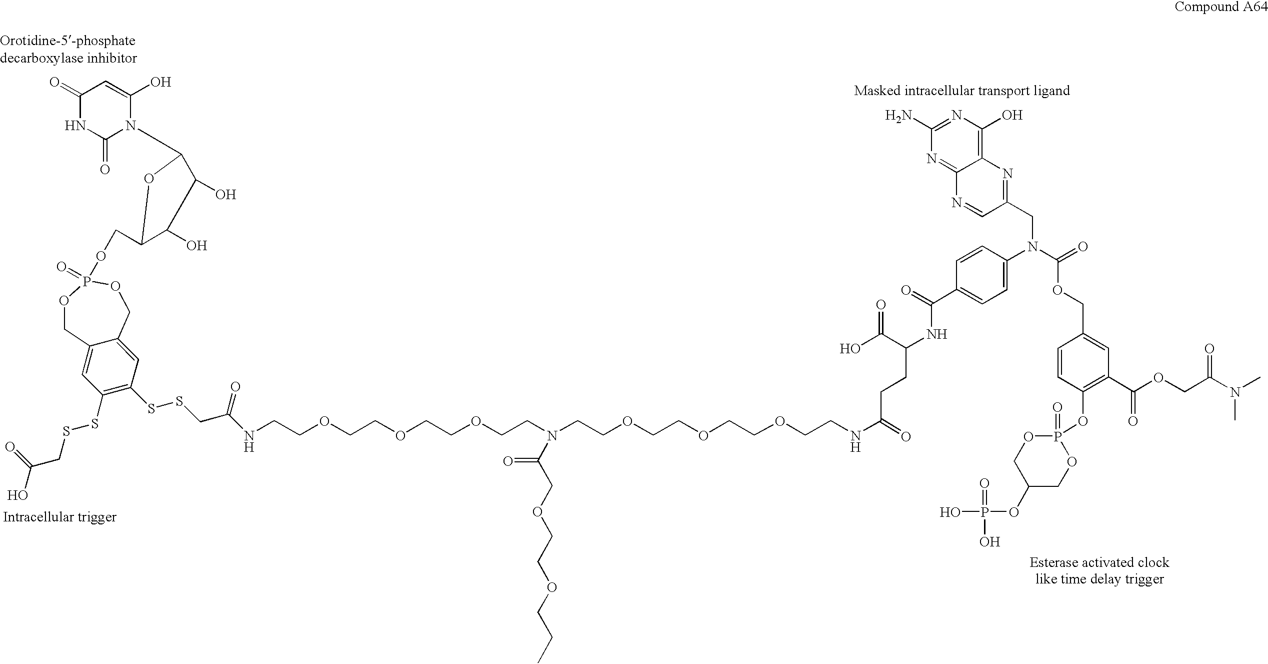

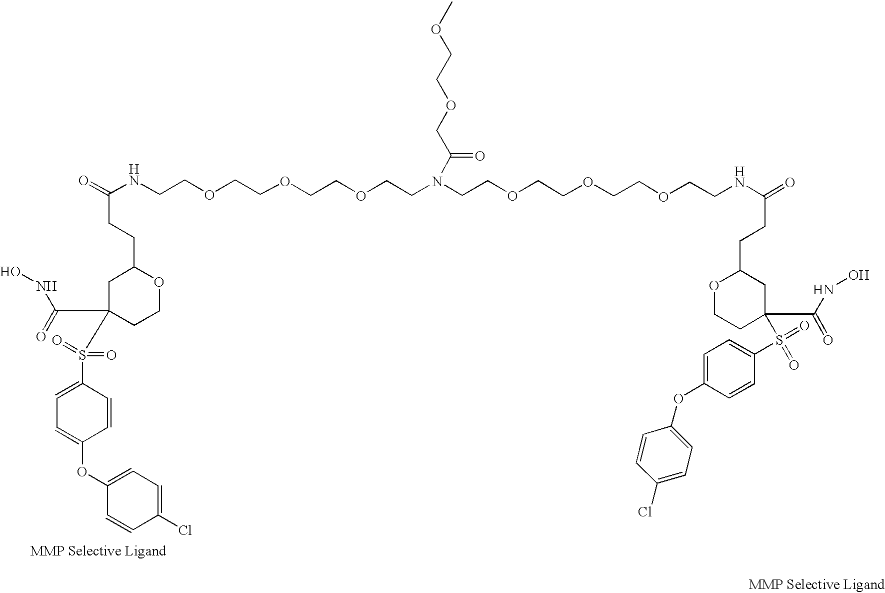

Compound A64 may be prepared by the method described for compound C62 by replacing compound C62.4 with compound A64.1.

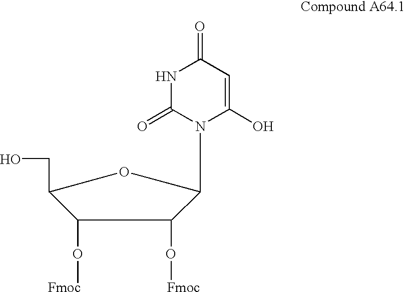

Compound A64.1 may be prepared by treating the parent nucleoside with one equivalent of trityl chloride and base in an inert solvent followed by two equivalents of 9-fluorenylmethyl chloroformate and a base such as pyridine, followed by treatment with acid to remove the 5′ trityl group. The following references relate to this subject matter: Levine H. L., et al., “Inhibition of Orotidine-5′-phosphate Decarboxylase by 1-(5′-Phospho-β-D-ribofuranosyl)barbituric Acid, 6-Azauridine 5′-Phosphate, and Uridine 5′-Phosphate,” Biochemistry, 19:4993-4999 (1980), the contents of which are incorporated herein by reference in their entirety.

### Example 65

Compound A65 has targeting ligands for urokinase, MMP (2, 3, 9, 12, or 13) and sigma receptors. The multifunctional delivery vehicle will deliver hydroxystaurosporine following intracellular transport and activation of an intracellular trigger by thiol reductases and cleavage of the phophate ester by phosphatases. Hydroxystaurosporine or UCN-01 is a potent inhibitor of protein kinases and exhibits synergistic toxicity with a wide range of antineoplastic compounds. The following references relate to this subject matter: Senderowicz A. M.; Sausville E. A., “Preclinical and Clinical Development of Cyclin-Dependent Kinase Modulators,” J Nat Cancer Institute, 92(5):376-387 (2000); Bunch R. T.; Eastman A., “Enhancement of Cisplatin-induced Cytotoxicity by 7-Hydroxystaurosporine (UCN-01), a New G₂-Checkpoint Inhibitor,” Clin Cancer Res, 2:791-797 (1996); Shao R. G., et al., “7-Hydroxystaurosporine (UCN-01) Induces Apoptosis in Human Colon Carcinoma and Leukemia Cells Independently of p53,” Exp Cell Res, 234:388-397 (1997); Monks A., et al., “UCN-01 Enhances the in Vitro Toxicity of Clinical Agents in Human Tumor Cell Lines,” Invest New Drugs, 18(2):95-107 (2000); Takahashi I., et al., “UCN-01 and UCN-02, New Selective Inhibitors of Protein Kinase C. II. Purification, Physico-chemical Properties, Structural Determination and Biological Activities,” J Antibiot, 42(4):571-6 (1989), the contents of which are incorporated herein by reference in their entirety.

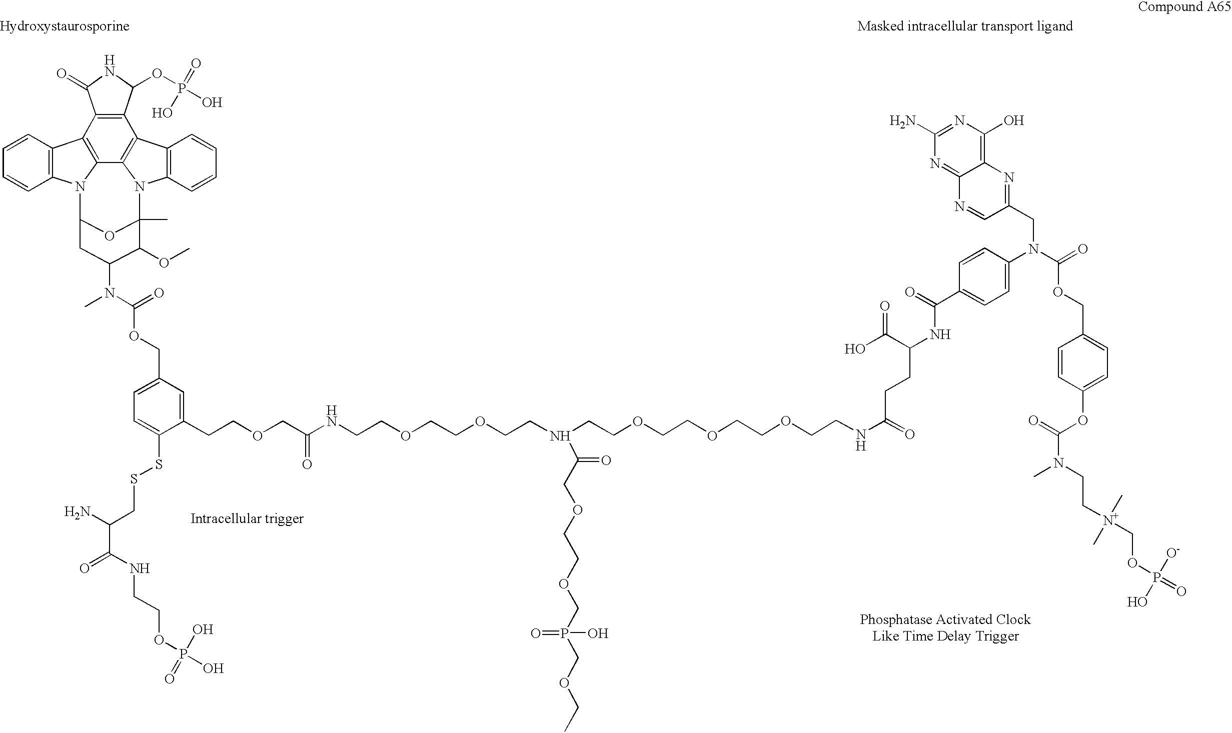

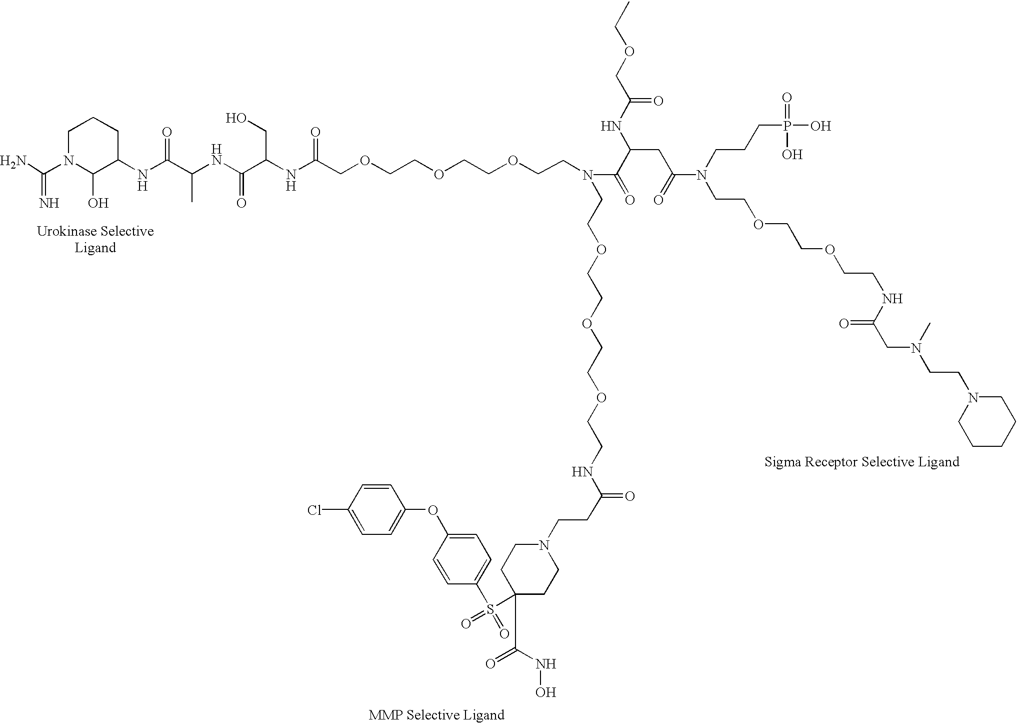

Compound A65 may be prepared by the methods described for compound 50 by replacing compound 50.1 with compound A65.1.

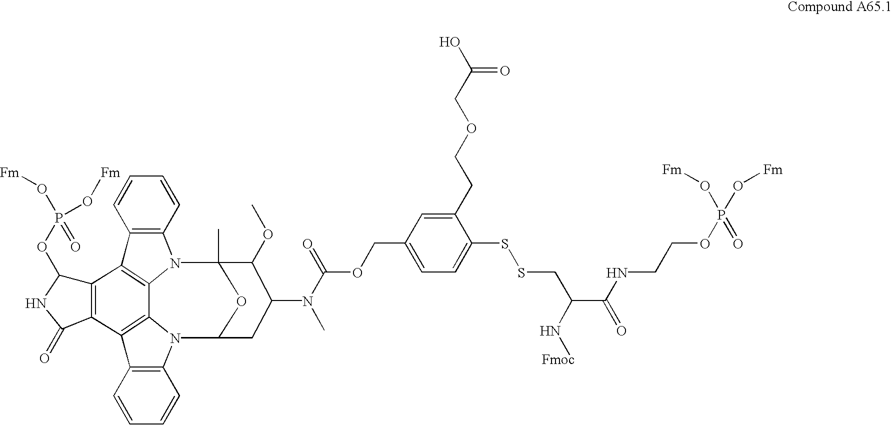

Compound A65.1 may be prepared by reacting compound A65.2 and compound 23.2b in an inert solvent in the presence of a base such as pyridine and then cleaving the Bsm ester with tris(2-aminoethyl)amine under conditions that will leave the Fmoc groups intact.

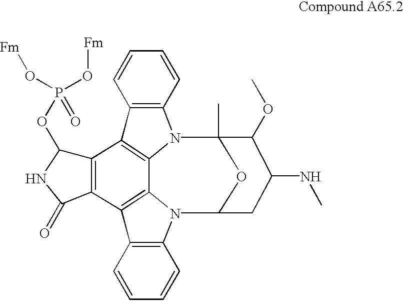

Compound A65.2 may be prepared by treating hydroxystaurosporine (UCN-01) with di-t-butyl pyrocarbonate and in an inert solvent and then reacting the product with phosphorochloridic acid bis-(9H-fluoren-9-ylmethyl) ester and a base such as triethylamine, and then treating with trifluroacetic acid to remove the t-Boc group.

What is claimed is:

1. An anticancer drug ET in which E comprises one or more effector agents that evoke tumor cell killing and T comprises:
a) A group referred to as a “tumor selective targeting ligand” which selectively binds to a target receptor that is increased on the surface of the tumor cell or in the microenvironment of the tumor cell compared to that for vital normal cells; and
b) One or more of the following:
I. A tumor selective targeting ligand;
II. A group, referred to as a “masked intracellular transport ligand” which can be modified in vivo to give a group referred to as an “intracellular transport ligand” which binds to a tumor cell receptor that actively transports bound ligands into the tumor cell;
III. A group referred to as a “trigger” that can be modified in vivo, wherein in vivo modification activates the trigger and modulates the pharmacological activity PA;
IV. A group referred to as an “intracellular trapping ligand”, which binds to one or more intracellular receptors or a group referred to as a “masked intracellular trapping ligand” which can be modified in vivo to give an “intracellular trapping ligand”;
and wherein, when a second targeting ligand is present in T then the first and second targeting ligands can bind simultaneously to two targeting receptor molecules;
and wherein when T consists of a targeting ligand and a trigger, and when in vivo modification of said trigger increases the tumor killing activity, the in vivo modification which activates said trigger, is caused by an enzyme or enzymatic activity that is increased at tumor cells or decreased at vital normal cells;
and wherein when T consists of a targeting ligand and a trigger, and when in vivo modification of said trigger decreases the tumor killing activity, the in vivo modification which activates said trigger, is caused by an enzyme or enzymatic activity that is decreased at tumor cells or increased at vital normal cells;
and provided that T is not an antibody, or an analog or component of an antibody, or a complex of antibodies, or a bispecific antibody, or an analog of a bispecific antibody, or a natural protein, or a complex of natural proteins, or a protein, or a naturally occurring polymer, or a radiolabelled dimer, or a polymer to which is attached, at multiple sites, one or more cytotoxic drugs.

2. An anticancer drug ET of claim 1 comprised of the following groups:
I. N1 targeting ligands, which may differ;
II. N2 masked intracellular transport ligands which may differ;
III. N3 triggers, which may differ, designated “detoxification triggers” wherein activation of the trigger decreases the toxicity of the drug;
IV. N4 effector agents which may differ;
V. N5 triggers which may differ, wherein activation of the trigger increases the toxicity of the drug;
VI. N6 intracellular trapping ligands or masked intracellular trapping ligands, which may differ;
and wherein:
N=1,2,3,4,5,6,7,8,9,10, or about 10; N2=0,1,2,3,4,5 or about 5; N3=0, 1, 2, 3, 4, 5, or about 5; N4=1, 2, 3, 4, 5, or about 5; N5=0, 1, 2, 3, 4, 5, 6, 7, 8, 9, 10, or about 10; N6=0, 1, 2, 3, 4, 5, 6, 7, 8, 9, 10, or about 10;

3. A compound of claim 2 in which:
N1=1,2, 3, or 4; N2=0, 1,or 2;
N3=0, 1, or 2; N4=1,2, or 3;
N5=0, 1, 2, or 3; N6=1, 2, or 3;

4. A compound of claim 3 wherein:
N1=1, N2=0, N3=1, N4=1, N5=0, and N6=0;
N1=1, N2=0, N3=0, N4=2, N5=0, and N6=0;
N1=1, N2=0, N3=0, N4=3, N5=0, and N6=0;
N1=1, N2=0, N3=0, N4=1, N5=1, and N6=0;
N1=1, N2=0, N3=0, N4=1, N5=2, and N6=0;
N1=1, N2=0, N3=0, N4=1, N5=3, and N6=0;
N1=1, N2=0, N3=0, N4=1, N5=0, and N6=1;
N1=1, N2=0, N3=1, N4=2, N5=0, and N6=0;
N1=1, N2=0, N3=1, N4=3, N5=0, and N6=0;
N1=1, N2=0, N3=1, N4=1, N5=1, and N6=0;
N1=1, N2=0, N3=1, N4=1, N5=2, and N6=0;
N1=1, N2=0, N3=1, N4=1, N5=3, and N6=0;
N1=1, N2=0, N3=1, N4=1, N5=0, and N6=1;
N1=1, N2=0, N3=1, N4=2, N5=1, and N6=0;
N1=1, N2=0, N3=1, N4=2 N5=0, and N6=1;
N1=1, N2=1, N3=1, N4=2, N5=2, and N6=0;
N1=1, N2=0, N3=1, N4=2, N5=2, and N6=1;
N1=1, N2=0, N3=1, N4=2, N5=3, and N6=0;
N1=1, N2=0, N3=1, N4=2, N5=3, and N6=1;
N1=1, N2=0, N3=1, N4=2, N5=0, and N6=1;
N1=1, N2=0, N3=1, N4=3, N5=1, and N6=0;
N1=1, N2=0, N3=1, N4=3, N5=1, and N6=1;
N1=1, N2=0, N3=1, N4=3, N5=2, and N6=0;
N1=1, N2=0, N3=1, N4=3, N5=2, and N6=1;
N1=1, N2=0, N3=1, N4=3, N5=3, and N6=0;
N1=1, N2=0, N3=1, N4=3, N5=3, and N6=1;
N1=1, N2=0, N3=1, N4=3, N5=0, and N6=1;
N1=1, N2=0, N3=1, N4=1, N5=1, and N6=1;
N1=1, N2=0, N3=1, N4=1, N5=2, and N6=1;
N1=1, N2=0, N3=1, N4=1, N5=3, and N6=1;
N1=1, N2=1, N3=0, N4=1, N5=0, and N6=0;
N1=1, N2=1, N3=0, N4=2, N5=0, and N6=0;
N1=1, N2=1, N3=0, N4=3, N5=0, and N6=0;
N1=1, N2=1, N3=0, N4=1, N5=1, and N6=0;
N1=1, N2=1, N3=0, N4=1, N5=2, and N6=0;
N1=1, N2=1, N3=0, N4=1, N5=3, and N6=0;
N1=1, N2=1, N3=0, N4=1, N5=0, and N6=1;
N1=1, N2=1, N3=0, N4=2, N5=1, and N6=0;
N1=1, N2=1, N3=0, N4=2, N5=1, and N6=1;
N1=1, N2=1, N3=0, N4=2, N5=2, and N6=0
N1=1, N2=1, N3=0, N4=2, N5=2, and N6=1;
N1=1, N2=1, N3=0, N4=2, N5=3, and N6=0;
N1=1, N2=1, N3=0, N4=2, N5=3, and N6=1;
N1=1, N2=1, N3=0, N4=2, N5=0, and N6=1;
N1=1, N2=1, N3=0, N4=3, N5=1, and N6=0;
N1=1, N2=1, N3=0, N4=3, N5=1, and N6=1;
N1=1, N2=1, N3=0, N4=3, N5=2, and N6=0;
N1=1, N2=1, N3=0, N4=3, N5=2, and N6=1;
N1=1, N2=1, N3=0, N4-3, N5=3, and N6=0;
N1=1, N2=1, N3=0, N4=3, N5=3, and N6=1;
N1=1, N2=1, N3=0, N4=3, N5=0, and N6=1;
N1=1, N2=1, N3=0, N4=1, N5=1, and N6=1;
N1=1, N2=1, N3=0, N4=1, N5=2, and N6=1;
N1=1, N2=1, N3=0, N4=1, N5=3, and N6=1;
N1=1, N2=1, N3=1, N4=1, N5=0, and N6=0;
N1=1, N2=1, N3=1, N4=2, N5=0, and N6=0;
N1=1, N2=1, N3=1, N4=3, N5=0, and N6=0;
N1=1, N2=1, N3=1, N4=1, N5=1, and N6=0;
N1=1, N2=1, N3=1, N4=1, N5=2, and N6=0;
N1=1, N2=1, N3=1, N4=1, N5=3, and N6=0;
N1=1, N2=1, N3=1, N4=1, N5=0, and N6=1;
N1=1,N2=1,N3=1,N4=2,N5=1, and N6=0;
N1=1,N2=1,N3=1,N4=2,N5=1, and N6=1;
N1=1, N2=1, N3=1, N4=2, N5=2, and N6=0;
N1=1, N2=1, N3=1, N4=2, N5=2, and N6=1;
N1=1, N2=1, N3=1, N4=2, N5=3, and N6=0
N1=1, N2=1, N3=1, N4=2, N5=3, and N6=1;
N1=1, N2=1, N3=1, N4=2, N5=0, and N6=0;
N1=1, N2=1, N3=1, N4=3, N5=1, and N6=0;
N1=1, N2=1, N3=1, N4=3, N5=1, and N6=1;
N1=1, N2=1, N3=1, N4=3, N5=2, and N6=0;
N1=1, N2=1, N3=1, N4=3, N5=2, and N6=1;
N1=1, N2=1, N3=1, N4=3, N5=3, and N6=0;
N1=1, N2=1, N3=1, N4=3, N5=3, and N6=1;
N1=1, N2=1, N3=1, N4=3, N5=0, and N6=1;
N1=1, N2=1, N3=1, N4=1, N5=1, and N6=1;
N1=1, N2=1, N3=1, N4=1, N5=2, and N6=1;
N1=1, N2=1, N3=1, N4=1, N5=3, and N6=1;
N1=1, N2=0, N3=0, N4=2, N5=1, and N6=0;
N1=1, N2=0, N3=0, N4=2, N5=2, and N6=0;
N1=1, N2=0, N3=0, N4=2, N5=3, and N6=0;
N1=1, N2=0, N3=0, N4=2, N5=0, and N6=1;
N1=1, N2=0, N3=0, N4=3, N5=1, and N6=0;
N1=1, N2=0, N3=0, N4=3, N5=2, and N6=0;
N1=1, N2=0, N3=0, N4=3, N5=3, and N6=0;
N1=1, N2=0, N3=0, N4=3, N5=0, and N6=1;
N1=1, N2=0, N3=0, N4=1, N5=2, and N6=1;
N1=1, N2=0, N3=0, N4=1, N5=3, and N6=1;
N1=1, N2=0, N3=0, N4=2, N5=1, and N6=1;
N1=1, N2=0, N3=0, N4=2, N5=2, and N6=1;
N1=1, N2=0, N3=0, N4=2, N5=3, and N6=1;
N1=1, N2=0, N3=0, N4=3, N5=1, and N6=1;
N1=1, N2=0, N3=0, N4=3, N5=2, and N6=1;
N1=1, N2=0, N3=0, N4=3, N5=3, and N6=1;
N1=1, N2=0, N3=0, N4=1, N5=1, and N6=1;
N1=2, N2=0, N3=0, N4=1, N5=0, and N6=0;
N1=2, N2=0, N3=1, N4=1, N5=0, and N6=0;
N1=2, N2=0, N3=0, N4=2, N5=0, and N6=0;
N1=2, N2=0, N3=0, N4=3, N5=0, and N6=0;
N1=2, N2=0, N3=0, N4=1, N5=1, and N6=0;
N1=2, N2=0, N3=0, N4=1, N5=2, and N6=0;
N1=2, N2=0, N3=0, N4=1, N5=3, and N6=0;
N1=2, N2=0, N3=0, N4=1, N5=0, and N6=1;
N1=2, N2=0, N3=1, N4=2, N5=0, and N6=0;
N1=2, N2=0, N3=1, N4=3, N5=0, and N6=0;
N1=2, N2=0, N3=1, N4=1, N5=1, and N6=0;
N1=2, N2=0, N3=1, N4=1, N5=2, and N6=0;
N1=2, N2=0, N3=1, N4=1, N5=3, and N6=0;
N1=2, N2=0, N3=1, N4=1, N5=0, and N6=1;
N1=2, N2=0, N3=1, N4=2, N5=1, and N6=0;
N1=2, N2=0, N3=1, N4=2, N5=1, and N6=1;
N1=2, N2=0, N3=1, N4=2, N5=2, and N6=0;
N1=2, N2=0, N3=1, N4=2, N5=2, and N6=1;
N1=2, N2=0, N3=1, N4=2, N5=3, and N6=0;
N1=2, N2=0, N3=1, N4=2, N5=3, and N6=1;
N1=2, N2=0, N3=1, N4=2, N5=0, and N6=1;
N1=2, N2=0, N3=1, N4=3, N5=1, and N6=0;
N1=2, N2=0, N3=1, N4=3, N5=1, and N6=1;
N1=2, N2=0, N3=1, N4=3, N5=2, and N6=0;
N1=2, N2=0, N3=1, N4=3, N5=2, and N6=1;
N1=2, N2=0, N3=1, N4=3, N5=3, and N6=0;
N1=2, N2=0, N3=1, N4=3, N5=3, and N6=1;
N1=2, N2=0, N3=1, N4=3, N5=0, and N6=1;
N1=2, N2=0, N3=1, N4=1, N5=1, and N6=1;
N1=2, N2=0, N3=1, N4=1, N5=2, and N6=1;
N1=2, N2=0, N3=1, N4=1, N5=3, and N6=1;
N1=2, N2=1, N3=0, N4=1, N5=0, and N6=0;
N1=2, N2=1, N3=0, N4=2, N5=0, and N6=0;
N1=2, N2=1, N3=0, N4=3, N5=0, and N6=0;
N1=2, N2=1, N3=0, N4=1, N5=1, and N6=0;
N1=2, N2=1, N3=0, N4=1, N5=2, and N6=0;
N1=2, N2=1, N3=0, N4=1, N5=3, and N6=0;
N1=2, N2=1, N3=0, N4=1, N5=0, and N6=1;
N1=2, N2=1, N3=0, N4=2, N5=1, and N6=0;
N1=2, N2=1, N3=0, N4=2, N5=1, and N6=1;
N1=2, N2=1, N3=0, N4=2, N5=2, and N6=0;
N1=2, N2=1, N3=0, N4=2, N5=2, and N6=1; N1=2, N2=1, N3=0 N4=2, N5=3, and N6=0;
N1=2, N2=1, N3=0, N4=2, N5=3, and N6=1;
N1=2, N2=1, N3=0, N4=2, N5=0, and N6=1;
N1=2, N2=1, N3=0, N4=3, N5=1, and N6=0;
N1=2, N2=1, N3=0, N4=3, N5=1, and N6=1;
N1=2, N2=1, N3=0, N4=3, N5=2, and N6=0;
N1=2, N2=1, N3=0, N4=3, N5=2, and N6=1;
N1=2, N2=1, N3=0, N4=3, N5=3, and N6=0;
N1=2, N2=1, N3=0, N4=3, N5=3, and N6=1;
N1=2, N2=1, N3=0, N4=3, N5=0, and N6=1;
N1=2, N2=1, N3=0, N4=1, N5=1, and N6=1;
N1=2, N2=1, N3=0, N4=1, N5=2, and N6=1;
N1=2, N2=1, N3=0, N4=1, N5=3, and N6=1;
N1=2, N2=1, N3=1, N4=1, N5=0, and N6=0;
N1=2, N2=1, N3=1, N4=2, N5=0, and N6=0;
N1=2, N2=1, N3=1, N4=3, N5=0, and N6=0;
N1=2, N2=1, N3=1, N4=1, N5=1, and N6=0;
N1=2, N2=1, N3=1, N4=1, N5=2, and N6=0;
N1=2, N2=1, N3=1, N4=1, N5=3, and N6=0;
N1=2, N2=1, N3=1, N4=1, N5=0, and N6=1;
N1=2, N2=1, N3=1, N4=2, N5=1, and N6=0;
N1=2, N2=1, N3=1, N4=2, N5=1, and N6=1;
N1=2, N2=1, N3=1, N4=2, N5=2, and N6=0;
N1=2, N2=1, N3=1, N4=2, N5=2, and N6=1;
N1=2, N2=1, N3=1, N4=2, N5=3, and N6=0;
N1=2, N2=1, N3=1, N4=2, N5=3, and N6=1;
N1=2, N2=1, N3=1, N4=2, N5=0, and N6=1;
N1=2, N2=1, N3=1, N4=3, N5=1, and N6=0;
N1=2, N2=1, N3=1, N4=3, N5=1, and N6=1;
N1=2, N2=1, N3=1, N4=3, N5=2, and N6=0;
N1=2, N2=1, N3=1, N4=3, N5=2, and N6=1;
N1=2, N2=1, N3=1, N4=3, N5=3, and N6=0;
N1=2, N2=1, N3=1, N4=3, N5=3, and N6=1;
N1=2, N2=1, N3=1, N4=3, N5=0, and N6=1;
N1=2, N2=1, N3=1, N4=1, N5=1, and N6=1;
N1=2, N2=1, N3=1, N4=1, N5=2, and N6=1;
N1=2, N2=1, N3=1, N4=1, N5=3, and N6=1;
N1=2, N2=0, N3=0, N4=2, N5=1, and N6=0;
N1=2, N2=0, N3=0, N4=2, N5=2, and N6=0;
N1=2, N2=0, N3=0, N4=2, N5=3, and N6=0
N1=2, N2=0, N3=0, N4=2, N5=0, and N6=1;
N1=2, N2=0, N3=0, N4=3, N5=1, and N6=0;
N1=2, N2=0, N3=0, N4=3, N5=2, and N6=0;
N1=2, N2=0, N3=0, N4=3, N5=3, and N6=0;
N1=2, N2=0, N3=0, N4=3, N5=0, and N6=1;
N1=2, N2=0, N3=0, N4=1, N5=2, and N6=1;
N1=2, N2=0, N3=0, N4=1, N5=3, and N6=1;
N1=2, N2=0, N3=0, N4=2, N5=1, and N6=1;
N1=2, N2=0, N3=0, N4=2, N5=2, and N6=1;
N1=2, N2=0, N3=0, N4=2, N5=3, and N6=1;
N1=2, N2=0, N3=0, N4=3, N5=1, and N6=1;
N1=2, N2=0, N3=0, N4=3, N5=2, and N6=1;
N1=2, N2=0, N3=0, N4=3, N5=3, and N6=1;
N1=2, N2=0, N3=0, N4=1, N5=1, and N6=1;
N1=3, N2=0, N3=0, N4=1, N5=0, and N6=0;
N1=3, N2=0, N3=1, N4=1, N5=0, and N6=0;
N1=3, N2=0, N3=0, N4=2, N5=0, and N6=0;
N1=3, N2=0, N3=0, N4=3, N5=0, and N6=0;
N1=3, N2=0, N3=0, N4=1, N5=1, and N6=0;
N1=3, N2=0, N3=0, N4=1, N5=2, and N6=0;
N1=3, N2=0, N3=0, N4=1, N5=3, and N6=0;
N1=3, N2=0, N3=0, N4=1, N5=0, and N6=1;
N1=3, N2=0, N3=1, N4=2, N5=0, and N6=0;
N1=3, N2=0, N3=1, N4=3, N5=0, and N6=0;
N1=3, N2=0, N3=1, N4=1, N5=1, and N6=0;
N1=3, N2=0, N3=1, N4=1, N5=2, and N6=0;
N1=3, N2=0, N3=1, N4=1, N5=3, and N6=0;
N1=3, N2=0, N3=1, N4=1, N5=0, and N6=1;
N1=3, N2=0, N3=1, N4=2, N5=1, and N6=0;
N1=3, N2=0, N3=1, N4=2, N5=1, and N6=1;
N1=3, N2=0, N3=1, N4=2, N5=2, and N6=0;
N1=3, N2=0, N3=1, N4=2, N5=2, and N6=1;
N1=3, N2=0, N3=1, N4=2, N5=3, and N6=0;
N1=3, N2=0, N3=1, N4=2, N5=3, and N6=1;
N1=3, N2=0, N3=1, N4=2, N5=0, and N6=1;
N1=3, N2=0, N3=1, N4=3, N5=1, and N6=0;
N1=3, N2=0, N3=1, N4=3, N5=1, and N6=1;
N1=3, N2=0, N3=1, N4=3, N5=2, and N6=0;
N1=3, N2=0, N3=1, N4=3, N5=2, and N6=1;
N1=3, N2=0, N3=1, N4=3, N5=3, and N6=0;
N1=3, N2=0, N3=1, N4=3, N5=3, and N6=1;
N1=3, N2=0, N3=1, N4=3, N5=0, and N6=1;
N1=3, N2=0, N3=1, N4=1, N5=1, and N6=1;
N1=3, N2=0, N3=1, N4=1, N5=2, and N6=1;
N1=3, N2=0, N3=1, N4=1, N5=3, and N6=1;
N1=3, N2=1, N3=0, N4=1, N5=0, and N6=0;
N1=3, N2=1, N3=0, N4=2, N5=0, and N6=0;
N1=3, N2=1, N3=0, N4=3, N5=0, and N6=0;
N1=3, N2=1, N3=0, N4=1, N5=1, and N6=0;
N1=3, N2=1, N3=0, N4=1, N5=2, and N6=0;
N1=3, N2=1, N3=0, N4=1, N5=3, and N6=0;
N1=3, N2=1, N3=0, N4=1, N5=0, and N6=1;
N1=3, N2=1, N3=0, N4=2, N5=1, and N6=0;
N1=3, N2=1, N3=0, N4=2, N5=1, and N6=1;
N1=3, N2=1, N3=0, N4=2, N5=2, and N6=0;
N1=3, N2=1, N3=0, N4=2, N5=2, and N6=1;
N1=3, N2=1, N3=0, N4=2, N5=3, and N6=0;
N1=3, N2=1, N3=0, N4=2, N5=3, and N6=1;
N1=3, N2=1, N3=0, N4=2, N5=0, and N6=1;
N1=3, N2=1, N3=0, N4=3, N5=1, and N6=0;
N1=3, N2=1, N3=0, N4=3, N5=1, and N6=1;
N1=3, N2=1, N3=0, N4=3, N5=2, and N6=0;
N1=3, N2=1, N3=0, N4=3, N5=2, and N6=1;
N1=3, N2=1, N3=0, N4=3, N5=3, and N6=0;
N1=3, N2=1, N3=0, N4=3, N5=3, and N6=1;
N1=3, N2=1, N3=0, N4=3, N5=0, and N6=1;
N1=3, N2=1, N3=0, N4=1, N5=1, and N6=1;
N1=3, N2=1, N3=0, N4=1, N5=2, and N6=1;
N1=3, N2=1, N3=0, N4=1, N5=3, and N6=1;
N1=3, N2=1, N3=1, N4=1, N5=0, and N6=0;
N1=3, N2=1, N3=1, N4=2, N5=0, and N6=0;
N1=3, N2=1, N3=1, N4=3, N5=0, and N6=0;
N1=3, N2=1, N3=1, N4=1, N5=1, and N6=0;
N1=3, N2=1, N3=1, N4=1, N5=2, and N6=0;
N1=3, N2=1, N3=1, N4=1, N5=3, and N6=0;
N1=3, N2=1, N3=1, N4=1, N5=0, and N6=1;
N1=3, N2=1, N3=1, N4=2, N5=1, and N6=0;
N1=3, N2=1, N3=1, N4=2, N5=1, and N6=1;
N1=3, N2=1, N3=1, N4=2, N5=2, and N6=0;
N1=3, N2=1, N3=1, N4=2, N5=2, and N6=1;
N1=3, N2=1, N3=1, N4=2, N5=3, and N6=0;
N1=3, N2=1, N3=1, N4=2, N5=3, and N6=1;
N1=3, N2=1, N3=1, N4=2, N5=0, and N6=1;
N1=3, N2=1, N3=1, N4=3, N5=1, and N6=0;
N1=3, N2=1, N3=1, N4=3, N5=1, and N6=1;
N1=3, N2=1, N3=1, N4=3, N5=2, and N6=0;
N1=3, N2=1, N3=1, N4=3, N5=2, and N6=1;
N1=3, N2=1, N3=1, N4=3, N5=3, and N6=0;
N1=3, N2=1, N3=1, N4=3, N5=3, and N6=1;
N1=3, N2=1, N3=1, N4=3, N5=0, and N6=1;
N1=3, N2=1, N3=1, N4=1, N5=1, and N6=1;
N1=3, N2=1, N3=1, N4=1, N5=2, and N6=1;
N1=3, N2=1, N3=1, N4=1, N5=3, and N6=1;
N1=3, N2=0, N3=0, N4=2, N5=1, and N6=0;
N1=3, N2=0, N3=0, N4=2, N5=2, and N6=0;
N1=3, N2=0, N3=0, N4=2, N5=3, and N6=0;
N1=3, N2=0, N3=0, N4=2, N5=0, and N6=1;
N1=3, N2=0, N3=0, N4=3, N5=1, and N6=0;
N1=3, N2=0, N3=0, N4=3, N5=2, and N6=0;
N1=3, N2=0, N3=0, N4=3, N5=3, and N6=0;
N1=3, N2=0, N3=0, N4=3, N5=0, and N6=1;
N1=3, N2=0, N3=0, N4=1, N5=2, and N6=1;
N1=3, N2=0, N3=0, N4=1, N5=3, and N6=1;
N1=3, N2=0, N3=0, N4=2, N5=1, and N6=1;
N1=3, N2=0, N3=0, N4=2, N5=2, and N6=1;
N1=3, N2=0, N3=0, N4=2, N5=3, and N6=1;
N1=3, N2=0, N3=0, N4=3, N5=1, and N6=1;
N1=3, N2=0, N3=0, N4=3, N5=2, and N6=1;
N1=3, N2=0, N3=0, N4=3, N5=3, and N6=1;
N1=3, N2=0, N3=0, N4=1, N5=1, and N6=1;
N1=4, N2=0, N3=0, N4=1, N5=0, and N6=0;
N1=4, N2=0, N3=1, N4=1, N5=0, and N6=0;
N1=4, N2=0, N3=0, N4=2, N5=0, and N6=0;
N1=4, N2=0, N3=0, N4=3, N5=0, and N6=0;
N1=4, N2=0, N3=0, N4=1, N5=1, and N6=0;
N1=4, N2=0, N3=0, N4=1, N5=2, and N6=0;
N1=4, N2=0, N3=0, N4=1, N5=3, and N6=0;
N1=4, N2=0, N3=0, N4=1, N5=0, and N6=1;
N1=4, N2=0, N3=1, N4=2, N5=0, and N6=0;
N1=4, N2=0, N3=1, N4=3, N5=0, and N6=0;
N1=4, N2=0, N3=1, N4=1, N5=1, and N6=0;
N1=4, N2=0, N3=1, N4=1, N5=2, and N6=0;
N1=4, N2=0, N3=1, N4=1, N5=3, and N6=0;
N1=4, N2=0, N3=1, N4=1, N5=0, and N6=1;
N1=4, N2=0, N3=1, N4=2, N5=1, and N6=0;
N1=4, N2=0, N3=1, N4=2, N5=1, and N6=1;
N1=4, N2=0, N3=1, N4=2, N5=2, and N6=0;
N1=4, N2=0, N3=1, N4=2, N5=2, and N6=1;
N1=4, N2=0, N3=1, N4=2, N5=3, and N6=0;
N1=4, N2=0, N3=1, N4=2, N5=3, and N6=1;
N1=4, N2=0, N3=1, N4=2, N5=0, and N6=1;
N1=4, N2=0, N3=1, N4=3, N5=1, and N6=0;
N1=4, N2=0, N3=1, N4=3, N5=1, and N6=1;
N1=4, N2=0, N3=1, N4=3, N5=2, and N6=0;
N1=4, N2=0, N3=1, N4=3, N5=2, and N6=1;
N1=4, N2=0, N3=1, N4=3, N5=3, and N6=0;
N1=4, N2=0, N3=1, N4=3, N5=3, and N6=1;
N1=4, N2=0, N3=1, N4=3, N5=0, and N6=1;
N1=4, N2=0, N3=1, N4=1, N5=1, and N6=1;
N1=4, N2=0, N3=1, N4=1, N5=2, and N6=1;
N1=4, N2=0, N3=1, N4=1, N5=3, and N6=1;
N1=4, N2=1, N3=0, N4=1, N5=0, and N6=0;
N1=4, N2=1, N3=0, N4=2, N5=0, and N6=0;
N1=4, N2=1, N3=0, N4=3, N5=0, and N6=0;
N1=4, N2=1, N3=0, N4=1, N5=1, and N6=0;
N1=4, N2=1, N3=0, N4=1, N5=2, and N6=0;
N1=4, N2=1, N3=0, N4=1, N5=3, and N6=0;
N1=4, N2=1, N3=0, N4=1, N5=0, and N6=1;
N1=4, N2=1, N3=0, N4=2, N5=1, and N6=0;
N1=4, N2=1, N3=0, N4=2, N5=1, and N6=1;
N1=4, N2=1, N3=0, N4=2, N5=2, and N6=0;
N1=4, N2=1, N3=0, N4=2, N5=2, and N6=1;
N1=4, N2=1, N3=0, N4=2, N5=3, and N6=0;
N1=4, N2=1, N3=0, N4=2, N5=3, and N6=1;
N1=4, N2=1, N3=0, N4=2, N5=0, and N6=1;
N1=4, N2=1, N3=0, N4=3, N5=1, and N6=0;
N1=4, N2=1, N3=0, N4=3, N5=1, and N6=1;
N1=4, N2=1, N3=0, N4=3, N5=2, and N6=0;
N1=4, N2=1, N3=0, N4=3, N5=2, and N6=1;
N1=4, N2=1, N3=0, N4=3, N5=3, and N6=0;
N1=4, N2=1, N3=0, N4=3, N5=3, and N6=1;
N1=4, N2=1, N3=0, N4=3, N5=0, and N6=1;
N1=4, N2=1, N3=0, N4=1, N5=1, and N6=1;
N1=4, N2=1, N3=0, N4=1, N5=2, and N6=1;
N1=4, N2=1, N3=0, N4=1, N5=3, and N6=1;
N1=4, N2=1, N3=1, N4=1, N5=0, and N6=0;
N1=4, N2=1, N3=1, N4=2, N5=0, and N6=0;
N1=4, N2=1, N3=1, N4=3, N5=0, and N6=0;
N1=4, N2=1, N3=1, N4=1, N5=1, and N6=0;
N1=4, N2=1, N3=1, N4=1, N5=2, and N6=0;
N1=4, N2=1, N3=1, N4=1, N5=3, and N6=0;
N1=4, N2=1, N3=1, N4=1, N5=0, and N6=1;
N1=4, N2=1, N3=1, N4=2, N5=1, and N6=0;
N1=4, N2=1, N3=1, N4=2, N5=1, and N6=1;
N1=4, N2=1, N3=1, N4=2, N5=2, and N6=0;
N1=4, N2=1, N3=1, N4=2, N5=2, and N6=1;
N1=4, N2=1, N3=1, N4=2, N5=3, and N6=0;
N1=4, N2=1, N3=1, N4=2, N5=3, and N6=1;
N1=4, N2=1, N3=1, N4=2, N5=0, and N6=1;
N1=4, N2=1, N3=1, N4=3, N5=1, and N6=0;
N1=4, N2=1, N3=1, N4=3, N5=1, and N6=1;
N1=4, N2=1, N3=1, N4=3, N5=2, and N6=0;
N1=4, N2=1, N3=1, N4=3, N5=2, and N6=1;
N1=4, N2=1, N3=1, N4=3, N5=3, and N6=0;
N1=4, N2=1, N3=1, N4=3, N5=3, and N6=1;
N1=4, N2=1, N3=1, N4=3, N5=0, and N6=1;
N1=4, N2=1, N3=1, N4=1, N5=1, and N6=1;
N1=4, N2=1, N3=1, N4=1, N5=2, and N6=1;
N1=4, N2=1, N3=1, N4=1, N5=3, and N6=1;
N1=4, N2=0, N3=0, N4=2, N5=1, and N6=0;
N1=4, N2=0, N3=0, N4=2, N5=2, and N6=0;
N1=4, N2=0, N3=0, N4=2, N5=3, and N6=0;
N1=4, N2=0, N3=0, N4=2, N5=0, and N6=1;
N1=4, N2=0, N3=0, N4=3, N5=1, and N6=0;
N1=4, N2=0, N3=0, N4=3, N5=2, and N6=0;
N1=4, N2=0, N3=0, N4=3, N5=3, and N6=0;
N1=4, N2=0, N3=0, N4=3, N5=0, and N6=1;
N1=4, N2=0, N3=0, N4=1, N5=2, and N6=1;
N1=4, N2=0, N3=0, N4=1, N5=3, and N6=1;
N1=4, N2=0, N3=0, N4=2, N5=1, and N6=1;
N1=4, N2=0, N3=0, N4=2, N5=2, and N6=1;
N1=4, N2=0, N3=0, N4=2, N5=3, and N6=1;
N1=4, N2=0, N3=0, N4=3, N5=1, and N6=1;
N1=4, N2=0, N3=0, N4=3, N5=2, and N6=1;
N1=4, N2=0, N3=0, N4=3, N5=3, and N6=1; or
N1=4, N2=0, N3=0, N4=1, N5=1, and N6=1;

5. An anticancer drug of claim 4 in which the targeting ligands selectively bind to target receptors on the surface of the tumor cell or in the microenvironment of the tumor cell, wherein the concentration of the target receptor is greater on the surface of the tumor cell or in the microenvironment of the tumor cell than on the surface or in the microenvironment of vital normal cells or normal cells.

6. A compound of claim 4, with two targeting ligands that selectively bind to target receptors on the surface of the tumor cell or in the microenvironment of the tumor cell, wherein the concentration of the target receptors is greater on the surface of the tumor cell or in the microenvironment of the tumor cell than on the surface or in the microenvironment of vital normal cells or normal cells.

7. A compound of claim 6 in which the targeting ligands are different and bind to different types of targeting receptors.

8. A compound of claim 4, with three targeting ligands that selectively bind to target receptors on the surface of the tumor cell or in the microenvironment of the tumor cell, wherein the concentration of the target receptors is greater on the surface of the tumor cell or in the microenvironment of the tumor cell than on the surface or in the microenvironment of vital normal cells or normal cells.

9. Anticancer drug of claim 4 comprised of two or more targeting ligands, wherein at least one of the targeting ligands binds to a target receptor on the surface of the target cell or in the microenvironment of the target cell, wherein the target has an increased amount of that target receptor compared to a nontarget cell that binds to a second targeting ligand of the compound.

10. A compound of claim 4 in which the effector agent is comprised of a cytotoxic drug.

11. A compound of claim 4 in which the effector agent is comprised of a radionuclide.

12. A compound of claim 4 in which the effector agent is comprised of a drug that stimulates the immune system.

13. A compound of claim 4 in which the effector agent is comprised of a group that can irreversibly chemically modify one or more tumor components.

14. A compound of claim 4 in which ET is comprised of an anticancer drug with two targeting ligands at least one of which binds to a target receptor selected from the following list:
1) a cathepsin type protease
2) a collagenase
3) a gelatinase
4) a matrix metalloproteinase
5) a membrane type matrix metalloproteinase
6) alpha v beta 3 integrin
7) bombesin/gastrin releasing peptide receptors
8) cathepsin B
9) cathepsin D
10) cathepsin K
11) cathepsin L
12) cathepsin O
13) fibroblast activation protein
14) folate binding receptors
15) gastrin/cholecystokinin type B receptor
16) glutamate carboxypeptidase II or (PSMA)
17) guanidinobenzoatase
18) laminin receptor
19) matrilysin
20) matripase
21) melanocyte stimulating hormone receptor
22) nitrobenzylthioinosine-binding receptors
23) norepenephrine transporters
24) nucleoside transporter proteins
25) peripheral benzodiazepam binding receptors
26) plasmin
27) seprase
28) sigma receptors
29) somatostatin receptors
30) stromelysin 3
31) trypsin
32) urokinase
33) MMP 1
34) MMP 2
35) MMP 3
36) MMP 7
37) MMP 9
38) Membrane type matrix metalloproteinase I
39) MMP 12
40) MMP 13

15. An anticancer drug of claim 4 comprised of one targeting ligand that binds the first target receptor (a1) and a second targeting ligand that binds to the second target receptor (a2) indicated in the pairs of (a1—a2) listed below: urokinase—a cathepsin type protease; urokinase—a collagenase; urokinase—a gelatinase; urokinase—a matrix metalloproteinase; urokinase—a membrane type matrix metalloproteinase; urokinase—alpha v beta 3 integrin; urokinase—bombesin/gastrin releasing peptide receptors; urokinase—cathepsin B; urokinase—cathepsin D; urokinase—to cathepsin K; urokinase—cathepsin L; urokinase—cathepsin O; urokinase—fibroblast activation protein; urokinase—folate binding receptors; urokinase—gastrin/cholecystokinin type B receptor; urokinase—glutamate carboxypeptidase II or (PSMA); urokinase—guanidinobenzoatase; urokinase—laminin receptor; urokinase—matrilysin; urokinase—matripase; urokinase—melanocyte stimulating hormone receptor; urokinase—nitrobenzylthioinosine-binding receptors or (nucleoside transporter); urokinase—norepinephrine transporters; urokinase—nucleoside transporter proteins; urokinase—peripheral benzodiazepam binding receptors; urokinase—plasmin; urokinase—seprase; urokinase—sigma receptors; urokinase—somatostatin receptors; urokinase—stromelysin 3; urokinase—trypsin; urokinase—urokinase; urokinase—MMP 1; urokinase—MMP 2; urokinase—MMP 3; urokinase—MMP 7; urokinase—MMP 9; urokinase—membrane type matrix metalloproteinase I; urokinase—MMP 12; urokinase—MMP 13; urokinase—a tumor antigen; plasmin—a cathepsin type protease; plasmin—a collagenase; plasmin—a gelatinase; plasmin—a matrix metalloproteinase; plasmin—a membrane type matrix metalloproteinase; plasmin—alpha v beta 3 integrin; plasmin—bombesin/gastrin releasing peptide receptors; plasmin—cathepsin B; plasmin—cathepsin D; plasmin—to cathepsin K; plasmin—cathepsin L; plasmin—cathepsin O; plasmin—fibroblast activation protein; plasmin—folate binding receptors; plasmin—gastrin/cholecystokinin type B receptor; plasmin—glutamate carboxypeptidase II or (PSMA); plasmin—guanidinobenzoatase; plasmin—laminin receptor; plasmin—matrilysin; plasmin—matripase; plasmin—melanocyte stimulating hormone receptor; plasmin—nitrobenzylthioinosine-binding receptors or (nucleoside transporter); plasmin—norepinephrine transporters; plasmin—nucleoside transporter proteins; plasmin—peripheral benzodiazepam binding receptors; plasmin—plasmin; plasmin—seprase; plasmin—sigma receptors; plasmin—somatostatin receptors; plasmin—stromelysin 3; plasmin—trypsin; plasmin—urokinase; plasmin—MMP 1; plasmin—MMP 2; plasmin—MMP 3; plasmin—MMP 7; plasmin—MMP 9; plasmin—membrane type matrix metalloproteinase I; plasmin—MMP 12; plasmin—MMP 13; plasmin—a tumor antigen; a collagenase—a cathepsin type protease; a collagenase—a collagenase; a collagenase—a gelatinase; a collagenase—a matrix metalloproteinase; a collagenase—a membrane type matrix metalloproteinase; a collagenase—alpha v beta 3 integrin; a collagenase—bombesin/gastrin releasing peptide receptors; a collagenase—cathepsin B; a collagenase—cathepsin D; a collagenase—to cathepsin K; a collagenase—cathepsin L; a collagenase—cathepsin O; a collagenase—fibroblast activation protein; a collagenase—folate binding receptors; a collagenase—gastrin/cholecystokinin type B receptor; a collagenase—glutamate carboxypeptidase II or (PSMA); a collagenase—guanidinobenzoatase; a collagenase—laminin receptor; a collagenase—matrilysin; a collagenase—-matripase; a collagenase—melanocyte stimulating hormone receptor; a collagenase—nitrobenzylthioinosine-binding receptors or (nucleoside transporter); a collagenase—norepinephrine transporters; a collagenase—nucleoside transporter proteins; a collagenase—peripheral benzodiazepam binding receptors; a collagenase—seprase; a collagenase—sigma receptors; a collagenase—somatostatin receptors; a collagenase—stromelysin 3; a collagenase—trypsin; a collagenase—a collagenase; a collagenase—MMP 1; a collagenase—MMP 2; a collagenase—MMP 3; a collagenase—MMP 7; a collagenase—MMP 9; a collagenase—membrane type matrix metalloproteinase I; a collagenase—MMP 12; a collagenase—MMP 13; a collagenase—a tumor antigen; a gelatinase—a cathepsin type protease; a gelatinase—a gelatinase; a gelatinase—a matrix metalloproteinase; a gelatinase—a membrane type matrix metalloproteinase; a gelatinase—alpha v beta 3 integrin; a gelatinase—bombesin/gastrin releasing peptide receptors; a gelatinase—cathepsin B; a gelatinase—cathepsin D; a gelatinase—to cathepsin K; a gelatinase—cathepsin L; a gelatinase—cathepsin O; a gelatinase—fibroblast activation protein; a gelatinase—folate binding receptors; a gelatinase—gastrin/cholecystokinin type B receptor; a gelatinase—glutamate carboxypeptidase II or (PSMA); a gelatinase—guanidinobenzoatase; a gelatinase—laminin receptor; a gelatinase—matrilysin; a gelatinase—matripase; a gelatinase—melanocyte stimulating hormone receptor; a gelatinase—nitrobenzylthioinosine-binding receptors or (nucleoside transporter); a gelatinase—norepinephrine transporters; a gelatinase—nucleoside transporter proteins; a gelatinase—peripheral benzodiazepam binding receptors; a gelatinase—seprase; a gelatinase—sigma receptors; a gelatinase—somatostatin receptors; a gelatinase—stromelysin 3; a gelatinase—trypsin; a gelatinase—MMP 1; a gelatinase—MMP 2; a gelatinase—MMP 3; a gelatinase—MMP 7; a gelatinase—MMP 9; a gelatinase—membrane type matrix metalloproteinase I; a gelatinase—MMP 12; a gelatinase—MMP 13; a gelatinase—a tumor antigen; a matrix metalloproteinase—a cathepsin type protease; a matrix metalloproteinase—a collagenase; a matrix metalloproteinase—a gelatinase; a matrix metalloproteinase—a matrix metalloproteinase; a matrix metalloproteinase—a membrane type matrix metalloproteinase; a matrix metalloproteinase—alpha v beta 3 integrin; a matrix metalloproteinase—bombesin/gastrin releasing peptide receptors; a matrix metalloproteinase—cathepsin B; a matrix metalloproteinase—cathepsin D; a matrix metalloproteinase—to cathepsin K; a matrix metalloproteinase—cathepsin L; a matrix metalloproteinase—cathepsin O; a matrix metalloproteinase—fibroblast activation protein; a matrix metalloproteinase—folate binding receptors; a matrix metalloproteinase—gastrin/cholecystokinin type B receptor; a matrix metalloproteinase—glutamate carboxypeptidase II or (PSMA); a matrix metalloproteinase—guanidinobenzoatase; a matrix metalloproteinase—laminin receptor; a matrix metalloproteinase—matrilysin; a matrix metalloproteinase—matripase; a matrix metalloproteinase—melanocyte stimulating hormone receptor; a matrix metalloproteinase—nitrobenzylthioinosine-binding receptors or (nucleoside transporter); a matrix metalloproteinase—norepinephrine transporters; a matrix metalloproteinase nucleoside transporter proteins; a matrix metalloproteinase—peripheral benzodiazepam binding receptors; a matrix metalloproteinase—plasmin; a matrix metalloproteinase—seprase; a matrix metalloproteinase—sigma receptors; a matrix metalloproteinase—somatostatin receptors; a matrix metalloproteinase—stromelysin 3; a matrix metalloproteinase—trypsin; a matrix metalloproteinase—a matrix metalloproteinase; a matrix metalloproteinase—MMP 1; a matrix metalloproteinase—MMP 2; a matrix metalloproteinase—MMP 3; a matrix metalloproteinase—MMP 7; a matrix metalloproteinase—MMP 9; a matrix metalloproteinase—membrane type matrix metalloproteinase I; a matrix metalloproteinase—MMP 12; a matrix metalloproteinase—MMP 13; a matrix metalloproteinase—a tumor antigen; a membrane type metalloproteinase—a cathepsin type protease; a membrane type metalloproteinase—a membrane type matrix metalloproteinase; a membrane type metalloproteinase—alpha v beta 3 integrin; a membrane type metalloproteinase—bombesin/gastrin releasing peptide receptors; a membrane type metalloproteinase—cathepsin B; a membrane type metalloproteinase—cathepsin D; a membrane type metalloproteinase—to cathepsin K; a membrane type metalloproteinase—cathepsin L; a membrane type metalloproteinase—cathepsin O; a membrane type metalloproteinase—fibroblast activation protein; a membrane type metalloproteinase—folate binding receptors; a membrane type metalloproteinase—gastrin/cholecystokinin type B receptor; a membrane type metalloproteinase—glutamate carboxypeptidase II or (PSMA); a membrane type metalloproteinase—guanidinobenzoatase; a membrane type metalloproteinase—laminin receptor; a membrane type metalloproteinase—matrilysin; a membrane type metalloproteinase—matripase; a membrane type metalloproteinase—melanocyte stimulating hormone receptor; a membrane type metalloproteinase nitrobenzylthioinosine-binding receptors or (nucleoside transporter); a membrane type metalloproteinase—norepinephrine transporters; a membrane type metalloproteinase—nucleoside transporter proteins; a membrane type metalloproteinase—peripheral benzodiazepam binding receptors; a membrane type metalloproteinase—seprase; a membrane type metalloproteinase—sigma receptors; a membrane type metalloproteinase—somatostatin receptors; a membrane type metalloproteinase—stromelysin 3; a membrane type metalloproteinase—trypsin; a membrane type metalloproteinase—MMP 1; a membrane type metalloproteinase—MMP 2; a membrane type metalloproteinase—MMP 3; a membrane type metalloproteinase—MMP 7; a membrane type metalloproteinase—MMP 9; a membrane type metalloproteinase—membrane type matrix metalloproteinase I; a membrane type metalloproteinase—MMP 12; a membrane type metalloproteinase—MMP 13; a membrane type metalloproteinase—a tumor antigen; alpha v beta 3 integrin—a cathepsin type protease; alpha v beta 3 integrin—alpha v beta 3 integrin; alpha v beta 3 integrin—bombesin/gastrin releasing peptide receptors; alpha v beta 3 integrin—cathepsin B; alpha v beta 3 integrin—cathepsin D; alpha v beta 3 integrin—cathepsin K; alpha v beta 3 integrin—cathepsin L; alpha v beta 3 integrin—cathepsin O; alpha v beta 3 integrin—fibroblast activation protein; alpha v beta 3 integrin—folate binding receptors; alpha v beta 3 integrin—gastrin/cholecystokinin type B receptor; alpha v beta 3 integrin—glutamate carboxypeptidase II or (PSMA); alpha v beta 3 integrin—guanidinobenzoatase; alpha v beta 3 integrin—laminin receptor; alpha v beta 3 integrin—matrilysin; alpha v beta 3 integrin—matripase; alpha v beta 3 integrin—melanocyte stimulating hormone receptor; alpha v beta 3 integrin—nitrobenzylthioinosine-binding receptors or (nucleoside transporter); alpha v beta 3 integrin—norepinephrine transporters; alpha v beta 3 integrin—nucleoside transporter proteins; alpha v beta 3 integrin—peripheral benzodiazepam binding receptors; alpha v beta 3 integrin—seprase; alpha v beta 3 integrin—sigma receptors; alpha v beta 3 integrin—somatostatin receptors; alpha v beta 3 integrin—stromelysin 3; alpha v beta 3 integrin—trypsin; alpha v beta 3 integrin—MMP 1; alpha v beta 3 integrin—MMP 2; alpha v beta 3 integrin—MMP 3; alpha v beta 3 integrin—MMP 7; alpha v beta 3 integrin—MMP 9; alpha v beta 3 integrin—membrane type matrix metalloproteinase I; alpha v beta 3 integrin—MMP 12; alpha v beta 3 integrin—MMP 13; alpha v beta 3 integrin—a tumor antigen; cathepsin B—a cathepsin type protease; cathepsin B—bombesin/gastrin releasing peptide receptors; cathepsin B—cathepsin B; cathepsin B—cathepsin D; cathepsin B—to cathepsin K; cathepsin B—cathepsin L; cathepsin B—cathepsin O; cathepsin B—fibroblast activation protein; cathepsin B—folate binding receptors; cathepsin B—gastrin/cholecystokinin type B receptor; cathepsin B—glutamate carboxypeptidase II or (PSMA); cathepsin B—guanidinobenzoatase; cathepsin B—laminin receptor; cathepsin B—matrilysin; cathepsin B—matripase; cathepsin B—melanocyte stimulating hormone receptor; cathepsin B—nitrobenzylthioinosine-binding receptors or (nucleoside transporter); cathepsin B—norepinephrine transporters; cathepsin B—nucleoside transporter proteins; cathepsin B—peripheral benzodiazepam binding receptors; cathepsin B—seprase; cathepsin B—sigma receptors; cathepsin B—somatostatin receptors; cathepsin B—stromelysin 3; cathepsin B—trypsin; cathepsin B—MMP 1; cathepsin B—MMP 2; cathepsin B—MMP 3; cathepsin B—MMP 7; cathepsin B—MMP 9; cathepsin B—membrane type matrix metalloproteinase I; cathepsin B—MMP 12; cathepsin B—MMP 13; cathepsin B—a tumor antigen; bombesin/gastrin releasing peptide receptors—a cathepsin type protease; bombesin/gastrin releasing peptide receptors—bombesin/gastrin releasing peptide receptors; bombesin/gastrin releasing peptide receptors—cathepsin B; bombesin/gastrin releasing peptide receptors—cathepsin D; bombesin/gastrin releasing peptide receptors—to cathepsin K; bombesin/gastrin releasing peptide receptors—cathepsin L; bombesin/gastrin releasing peptide receptors—cathepsin O; bombesin/gastrin releasing peptide receptors—fibroblast activation protein; bombesin/gastrin releasing peptide receptors—folate binding receptors; bombesin/gastrin releasing peptide receptors—gastrin/cholecystokinin type B receptor; bombesin/gastrin releasing peptide receptors—glutamate carboxypeptidase II or (PSMA); bombesin/gastrin releasing peptide receptors—guanidinobenzoatase; bombesin/gastrin releasing peptide receptors—laminin receptor; bombesin/gastrin releasing peptide receptors—matrilysin; bombesin/gastrin releasing peptide receptors—matripase; bombesin/gastrin releasing peptide receptors—melanocyte stimulating hormone receptor; bombesin/gastrin releasing peptide receptors—nitrobenzylthioinosine-binding receptors or (nucleoside transporter); bombesin/gastrin releasing peptide receptors—norepinephrine transporters; bombesin/gastrin releasing peptide receptors—nucleoside transporter proteins; bombesin/gastrin releasing peptide receptors—peripheral benzodiazepam binding receptors; bombesin/gastrin releasing peptide receptors—seprase; bombesin/gastrin releasing peptide receptors—sigma receptors; bombesin/gastrin releasing peptide receptors—somatostatin receptors; bombesin/gastrin releasing peptide receptors—stromelysin 3; bombesin/gastrin releasing peptide receptors—trypsin; bombesin/gastrin releasing peptide receptors—MMP 1; bombesin/gastrin releasing peptide receptors—MMP 2; bombesin/gastrin releasing peptide receptors—MMP 3; bombesin/gastrin releasing peptide receptors—MMP 7; bombesin/gastrin releasing peptide receptors—MMP 9; bombesin/gastrin releasing peptide receptors—membrane type matrix metalloproteinase I; bombesin/gastrin releasing peptide receptors—MMP 12; bombesin/gastrin releasing peptide receptors—MMP 13; bombesin/gastrin releasing peptide receptors—a tumor antigen; fibroblast activation protein—a cathepsin type protease; fibroblast activation protein—cathepsin D; fibroblast activation protein—to cathepsin K; fibroblast activation protein—cathepsin L; fibroblast activation protein—cathepsin O; fibroblast activation protein—fibroblast activation protein; fibroblast activation protein—folate binding receptors; fibroblast activation protein—gastrin/cholecystokinin type B receptor; fibroblast activation protein—glutamate carboxypeptidase II or (PSMA); fibroblast activation protein—guanidinobenzoatase; fibroblast activation protein—laminin receptor; fibroblast activation protein—matrilysin; fibroblast activation protein—matripase; fibroblast activation protein—melanocyte stimulating hormone receptor; fibroblast activation protein—nitrobenzylthioinosine-binding receptors or (nucleoside transporter); fibroblast activation protein—norepinephrine transporters; fibroblast activation protein—nucleoside transporter proteins; fibroblast activation protein—peripheral benzodiazepam binding receptors; fibroblast activation protein—plasmin; fibroblast activation protein—seprase; fibroblast activation protein—sigma receptors; fibroblast activation protein—somatostatin receptors; fibroblast activation protein—stromelysin 3; fibroblast activation protein—trypsin; fibroblast activation protein—MMP 1; fibroblast activation protein—MMP 2; fibroblast activation protein—MMP 3; fibroblast activation protein—MMP 7; fibroblast activation protein—MMP 9; fibroblast activation protein—membrane type matrix metalloproteinase I; fibroblast activation protein—MMP 12; fibroblast activation protein—MMP 13; fibroblast activation protein—a tumor antigen; glutamate carboxypeptidase II or PSMA—cathepsin D; glutamate carboxypeptidase II or PSMA—to cathepsin K; glutamate carboxypeptidase II or PSMA—cathepsin L; glutamate carboxypeptidase II or PSMA—cathepsin O; glutamate carboxypeptidase II or PSMA—fibroblast activation protein; glutamate carboxypeptidase II or PSMA—folate binding receptors; glutamate carboxypeptidase II or PSMA—gastrin/cholecystokinin type B receptor; glutamate carboxypeptidase II or PSMA—glutamate carboxypeptidase II or (PSMA); glutamate carboxypeptidase II or PSMA—guanidinobenzoatase; glutamate carboxypeptidase II or PSMA—laminin receptor; glutamate carboxypeptidase II or PSMA—matrilysin; glutamate carboxypeptidase II or PSMA—matripase; glutamate carboxypeptidase II or PSMA—melanocyte stimulating hormone receptor; glutamate carboxypeptidase II or PSMA—nitrobenzylthioinosine-binding receptors or (nucleoside transporter); glutamate carboxypeptidase II or PSMA—nucleoside transporter proteins; glutamate carboxypeptidase II or PSMA—peripheral benzodiazepam binding receptors; glutamate carboxypeptidase II or PSMA—seprase; glutamate carboxypeptidase II or PSMA—sigma receptors; glutamate carboxypeptidase II or PSMA—somatostatin receptors; glutamate carboxypeptidase II or PSMA—stromelysin 3; glutamate carboxypeptidase II or PSMA—trypsin; glutamate carboxypeptidase II or PSMA—MMP 1; glutamate carboxypeptidase II or PSMA—MMP 2; glutamate carboxypeptidase II or PSMA—MMP 3; glutamate carboxypeptidase II or PSMA—MMP 7; glutamate carboxypeptidase II or PSMA—MMP 9; glutamate carboxypeptidase II or PSMA—membrane type matrix metalloproteinase I; glutamate carboxypeptidase II or PSMA—MMP 12; glutamate carboxypeptidase II or PSMA—MMP 13; glutamate carboxypeptidase II or PSMA—a tumor antigen; laminin receptor—a cathepsin type protease; laminin receptor—cathepsin B; laminin receptor—cathepsin D; laminin receptor—to cathepsin K; laminin receptor—cathepsin L; laminin receptor—cathepsin O; laminin receptor—fibroblast activation protein; laminin receptor—folate binding receptors; laminin receptor—gastrin/cholecystokinin type B receptor; laminin receptor—guanidinobenzoatase; laminin receptor—laminin receptor; laminin receptor—matrilysin; laminin receptor—matripase; laminin receptor—melanocyte stimulating hormone receptor; laminin receptor—nitrobenzylthioinosine-binding receptors or (nucleoside transporter); laminin receptor—norepinephrine transporters; laminin receptor—nucleoside transporter proteins; laminin receptor—peripheral benzodiazepam binding receptors; laminin receptor—seprase; laminin receptor—sigma receptors; laminin receptor—somatostatin receptors; laminin receptor—stromelysin 3; laminin receptor—trypsin; laminin receptor—MMP 1; laminin receptor—MMP 2; laminin receptor—MMP 3; laminin receptor—MMP 7; laminin receptor—MMP 9; laminin receptor—membrane type matrix metalloproteinase I; laminin receptor—MMP 12; laminin receptor—MMP 13; laminin receptor—a tumor antigen; seprase—a cathepsin type protease; seprase—cathepsin D; seprase—to cathepsin K; seprase—cathepsin L; seprase—cathepsin O; seprase—fibroblast activation protein; seprase—folate binding receptors; seprase—gastrin/cholecystokinin type B receptor; seprase—guanidinobenzoatase; seprase—matripase; seprase—melanocyte stimulating hormone receptor; seprase—nitrobenzylthioinosine-binding receptors or (nucleoside transporter); seprase—norepinephrine transporters; seprase—nucleoside transporter proteins; seprase—peripheral benzodiazepam binding receptors; seprase—seprase; seprase—sigma receptors; seprase—somatostatin receptors; seprase—stromelysin 3; seprase—trypsin; seprase—MMP 1; seprase—MMP 2; seprase—MMP 3; seprase—MMP 7; seprase—MMP 9; seprase—membrane type matrix metalloproteinase I; seprase—MMP 12; seprase—MMP 13; seprase—a tumor antigen; guanidinobenzoatase—a cathepsin type protease; guanidinobenzoatase—cathepsin D; guanidinobenzoatase—to cathepsin K; guanidinobenzoatase—cathepsin L; guanidinobenzoatase—cathepsin O; guanidinobenzoatase—fibroblast activation protein; guanidinobenzoatase—folate binding receptors; guanidinobenzoatase—gastrin/cholecystokinin type B receptor; guanidinobenzoatase—guanidinobenzoatase; guanidinobenzoatase—matripase; guanidinobenzoatase—melanocyte stimulating hormone receptor; guanidinobenzoatase—nitrobenzylthioinosine-binding receptors or (nucleoside transporter); guanidinobenzoatase—norepinephrine transporters; guanidinobenzoatase—nucleoside transporter proteins; guanidinobenzoatase—peripheral benzodiazepam binding receptors; guanidinobenzoatase—sigma receptors; guanidinobenzoatase—somatostatin receptors; guanidinobenzoatase—stromelysin 3; guanidinobenzoatase—trypsin; guanidinobenzoatase—MMP 1; guanidinobenzoatase—MMP 2; guanidinobenzoatase—MMP 3; guanidinobenzoatase—MMP 7; guanidinobenzoatase—MMP 9; guanidinobenzoatase—membrane type matrix metalloproteinase I; guanidinobenzoatase—MMP 12; guanidinobenzoatase—MMP 13; guanidinobenzoatase—a tumor antigen; peripheral benzodiazepam binding receptors—a cathepsin type protease; peripheral benzodiazepam binding receptors—cathepsin D; peripheral benzodiazepam binding receptors—to cathepsin K; peripheral benzodiazepam binding receptors—cathepsin L; peripheral benzodiazepam binding receptors—cathepsin O; peripheral benzodiazepam binding receptors—fibroblast activation protein; peripheral benzodiazepam binding receptors—folate binding receptors; peripheral benzodiazepam binding receptors—gastrin/cholecystokinin type B receptor; peripheral benzodiazepam binding receptors—guanidinobenzoatase; peripheral benzodiazepam binding receptors—matripase; peripheral benzodiazepam binding receptors—melanocyte stimulating hormone receptor; peripheral benzodiazepam binding receptors—nitrobenzylthioinosine-binding receptors or (nucleoside transporter); peripheral benzodiazepam binding receptors—norepinephrine transporters; peripheral benzodiazepam binding receptors—nucleoside transporter proteins; peripheral benzodiazepam binding receptors—peripheral benzodiazepam binding receptors; peripheral benzodiazepam binding receptors—sigma receptors; peripheral benzodiazepam binding receptors-somatostatin receptors; peripheral benzodiazepam binding receptors—stromelysin 3; peripheral benzodiazepam binding receptors—trypsin; peripheral benzodiazepam binding receptors—MMP 1; peripheral benzodiazepam binding receptors—MMP 2; peripheral benzodiazepam binding receptors—MMP 3; peripheral benzodiazepam binding receptors—MMP 7; peripheral benzodiazepam binding receptors—MMP 9; peripheral benzodiazepam binding receptors—membrane type matrix metalloproteinase I; peripheral benzodiazepam binding receptors—MMP 12; peripheral benzodiazepam binding receptors—MMP 13; peripheral benzodiazepam binding receptors—a tumor antigen; folate binding receptors—a cathepsin type protease; folate binding receptors—cathepsin D; folate binding receptors—to cathepsin K; folate binding receptors—cathepsin L; folate binding receptors—cathepsin O; folate binding receptors—fibroblast activation protein; folate binding receptors folate binding receptors; folate binding receptors—matripase; folate binding receptors—melanocyte stimulating hormone receptor; folate binding receptors—nitrobenzylthioinosine-binding receptors or (nucleoside transporter); folate binding receptors—norepinephrine transporters; folate binding receptors—nucleoside transporter proteins; folate binding receptors—sigma receptors; folate binding receptors—somatostatin receptors; folate binding receptors—stromelysin 3; folate binding receptors—trypsin; folate binding receptors—MMP1; folatebindingreceptors—MMP2; folatebindingreceptors—MMP3; folate binding receptors—MMP 7; folate binding receptors—MMP 9; folate binding receptors—membrane type matrix metalloproteinase I; folate binding treceptors—MMP 12; folate binding receptors—MMP folatebinding receptors—a tumor antigen; folate binding receptors—a cathepsin type protease; folate binding receptors—cathepsin D; folate binding receptors—to cathepsin K; folate binding receptors—cathepsin L; folate binding receptors—cathepsin O; folate binding receptors—fibroblast activation protein; folate binding receptors folate binding receptors; folate binding receptors—matripase; folate binding receptors—melanocyte stimulating hormone receptor; folate binding receptors—nitrobenzylthioinosine-binding receptors or (nucleoside transporter); folate binding receptors—norepinephrine transporters; folate binding receptors—nucleoside transporter proteins; folate binding receptors—sigma receptors; folate binding receptors—somatostatin receptors; folate binding receptors—stromelysin 3; folate binding receptors—trypsin; folate binding receptors—MMP1; folate binding receptors—MMP 2; folate binding receptors—MMP3; folate binding receptors—MMP 7; folate binding receptors—MMP 9; folate binding receptors—membrane type matrix metalloproteinase I; folate binding receptors—MMP12; folate binding receptors—MMP13; folate binding receptors—a tumor antigen; nucleoside transporter proteins—a cathepsin type protease; nucleoside transporter proteins—cathepsin D; nucleoside transporter proteins—to cathepsin K; nucleoside transporter proteins—cathepsin L; nucleoside transporter proteins—cathepsin O; nucleoside transporter proteins—fibroblast activation protein; nucleoside transporter proteins—nucleoside transporter proteins; nucleoside transporter proteins—matripase; nucleoside transporter proteins—melanocyte stimulating hormone receptor; nucleoside transporter proteins—nitrobenzylthioinosine-binding receptors or (nucleoside transporter); nucleoside transporter proteins—norepinephrine transporters; nucleoside transporter proteins—nucleoside transporter proteins; nucleoside transporter proteins—sigma receptors; nucleoside transporter proteins—somatostatin receptors; nucleoside transporter proteins—stromelysin 3; nucleoside transporter proteins—trypsin; nucleoside transporter proteins—MMP 1; nucleoside transporter proteins—MMP 2; nucleoside transporter proteins—MMP 3; nucleoside transporter proteins—MMP 7; nucleoside transporter proteins—MMP 9; nucleoside transporter proteins—membrane type matrix metalloproteinase I; nucleoside transporter proteins—MMP 12; nucleoside transporter proteins—MMP 13; nucleoside transporter proteins—a tumor antigen; melanocyte stimulating hormone receptor—a cathepsin type protease; melanocyte stimulating hormone receptor—cathepsin D; melanocyte stimulating hormone receptor—to cathepsin K; melanocyte stimulating hormone receptor—cathepsin L; melanocyte stimulating hormone receptor—cathepsin O; melanocyte stimulating hormone receptor—fibroblast activation protein; melanocyte stimulating hormone receptor—melanocyte stimulating hormone receptor; melanocyte stimulating hormone receptor—melanocyte stimulating hormone receptor; melanocyte stimulating hormone receptor—nitrobenzylthioinosine-binding receptors or (nucleoside transporter); melanocyte stimulating hormone receptor—norepinephrine transporters; melanocyte stimulating hormone receptor—nucleoside transporter proteins; melanocyte stimulating hormone receptor—sigma receptors; melanocyte stimulating hormone receptor—somatostatin receptors; melanocyte stimulating hormone receptor—stromelysin 3; melanocyte stimulating hormone receptor—trypsin; melanocyte stimulating hormone receptor—MMP 1; melanocyte stimulating hormone receptor—MMP 2; melanocyte stimulating hormone receptor—MMP 3; melanocyte stimulating hormone receptor—MMP 7; melanocyte stimulating hormone receptor—MMP 9; melanocyte stimulating hormone receptor—membrane type matrix metalloproteinase I; melanocyte stimulating hormone receptor—MMP 12; melanocyte stimulating hormone receptor—MMP 13; melanocyte stimulating hormone receptor—a tumor antigen; sigma receptors—a cathepsin type protease; sigma receptors—cathepsin D; sigma receptors—to cathepsin K; sigma receptors—cathepsin L; sigma receptors—cathepsin O; sigma receptors—fibroblast activation protein; sigma receptors—sigma receptors; sigma receptors—matripase; sigma receptors—norepinephrine transporters; sigma receptors—sigma receptors; sigma receptors—somatostatin receptors; sigma receptors—stromelysin 3; sigma receptors—trypsin; sigma receptors—MMP 1; sigma receptors—MMP 2; sigma receptors—MMP 3; sigma receptors—MMP 7; sigma receptors—MMP 9; sigma receptors—membrane type matrix metalloproteinase I; sigma receptors—MMP 12; sigma receptors—MMP 13; sigma receptors—a tumor antigen; somatostatin receptors—a cathepsin type protease; somatostatin receptors—cathepsin D; somatostatin receptors—to cathepsin K; somatostatin receptors—cathepsin L; somatostatin receptors—cathepsin O; somatostatin receptors—fibroblast activation protein; somatostatin receptors—somatostatin receptors; somatostatin receptors matripase; somatostatin receptors—melanocyte stimulating hormone receptor; somatostatin receptors—sigma receptors; somatostatin receptors—somatostatin receptors; somatostatin receptors—stromelysin 3; somatostatin receptors—trypsin; somatostatin receptors—MMP 1; somatostatin receptors—MMP 2; somatostatin receptors—MMP 3; somatostatin receptors—MMP 7; somatostatin receptors—MMP 9; somatostatin receptors—membrane type matrix metalloproteinase I; somatostatin receptors—MMP 12; somatostatin receptors—MMP 13; somatostatin receptors—a tumor antigen; stromelysin 3—a cathepsin type protease; stromelysin 3—cathepsin D; stromelysin 3—to cathepsin K; stromelysin 3—cathepsin L; stromelysin 3—cathepsin O; stromelysin 3—fibroblast activation protein; stromelysin 3—stromelysin 3; stromelysin 3—matripase; stromelysin 3—melanocyte stimulating hormone receptor; stromelysin 3—somatostatin receptors; stromelysin 3—trypsin; stromelysin 3—MMP1; stromelysin 3—MMP 2; stromelysin 3—MMP 3; stromelysin 3—MMP7; stromelysin 3—MMP9; stromelysin 3—membrane type matrix metalloproteinase I; stromelysin 3—MMP 12; stromelysin 3—MMP 13; stromelysin 3—a tumor antigen; trypsin—a cathepsin type protease; trypsin—cathepsin D; trypsin—to cathepsin K; trypsin—cathepsin L; trypsin—cathepsin O; trypsin—fibroblast activation protein; trypsin—trypsin; trypsin—matripase; trypsin—melanocyte stimulating hormone receptor; trypsin—stromelysin 3; trypsin—MMP 1; trypsin—MMP 2; trypsin—MMP 3; trypsin—MMP 7; trypsin—MMP 9; trypsin—membrane type matrix metalloproteinase I; trypsin—MMP 12; trypsin—MMP 13; trypsin—a tumor antigen; MMP 1—a cathepsin type protease; MMP 1—cathepsin D; MMP 1—to cathepsin K; MMP 1—cathepsin L; MMP 1—cathepsin O; MMP 1—fibroblast activation protein; MMP 1—matripase; MMP 1—melanocyte stimulating hormone receptor; MMP 1—stromelysin 3; MMP 1—MMP 1; MMP 1—MMP2; MMP1—MMP 3; MMP 1—MMP 7; MMP 1—MMP9; MMP 1—membrane type matrix metalloproteinase I; MMP 1—MMP 12; MMP 1—MMP 13; MMP 1—a tumor antigen; MMP-2—a cathepsin type protease; MMP-2—cathepsin D; MMP-2—to cathepsin K; MMP-2—cathepsin L; MMP-2—cathepsin O; MMP-2—fibroblast activation protein; MMP-2—matripase; MMP-2—melanocyte stimulating hormone receptor; MMP-2—stromelysin 3; MMP-2—MMP 2; MMP-2—MMP 3; MMP-2—MMP 7; MMP-2—MMP 9; MMP-2—membrane type matrix metalloproteinase I; MMP-2—MMP-2; MMP-2—MMP-3; MMP-2—a tumor antigen; MMP-3—a cathepsin type protease; MMP-3—cathepsin D; MMP-3—to cathepsin K; MMP-3—cathepsin L; MMP-3—cathepsin O; MMP-3—matripase; MMP-3—MMP 3; MMP-3—MMP 7; MMP-3—MMP 9; MMP-3—membrane type matrix metalloproteinase I; MMP-3—MMP-3; MMP-3—a tumor antigen; MMP 7—a cathepsin type protease; MMP 7—cathepsin D; MMP 7—to cathepsin K; MMP 7—cathepsin L; MMP 7—cathepsin O; MMP 7—fibroblast activation protein; MMP 7—matripase; MMP 7—stromelysin 3; MMP 7—MMP 7; MMP 7—MMP 9; MMP 7—membrane type matrix metalloproteinase I; MMP 7—a tumor antigen; MMP 9—a cathepsin type protease; MMP 9—cathepsin D; MMP 9—to cathepsin K; MMP 9—cathepsin L; MMP 9—cathepsin O; MMP 9—matripase; MMP 9—MMP 9; MMP 9—membrane type matrix metalloproteinase I; MMP 9—a tumor antigen; MMP 12—a cathepsin type protease; MMP 12—cathepsin D; MMP 12—to cathepsin K; MMP 12—cathepsin L; MMP 12—cathepsin O; MMP 12—matripase; MMP 12—MMP 2; MMP 12—membrane type matrix metalloproteinase I; MMP 12—a tumor antigen; MMP 13—a cathepsin type protease; MMP 13—cathepsin D; MMP 13—to cathepsin K; MMP 13—cathepsin L; MMP 13—cathepsin O; MMP 13—matripase; MMP 13—membrane type matrix metalloproteinase I; MMP 13—a tumor antigen; Membrane type matrix metalloproteinase—a cathepsin type protease; Membrane type matrix metalloproteinase—cathepsin D; Membrane type matrix metalloproteinase—to cathepsin K; Membrane type matrix metalloproteinase—cathepsin L; Membrane type matrix metalloproteinase—cathepsin O; Membrane type matrix metalloproteinase—matripase; Membrane type matrix metalloproteinase—membrane type matrix metalloproteinase I; and Membrane type matrix metalloproteinase—a tumor antigen.

16. A compound of claim 15 that is also comprised of a third targeting ligand receptor that binds to a receptor that is present at increased amounts at a tumor cell compared to at a normal cell.

17. A compound of claim 16 in which the third targeting ligand binds to PSMA or glutamate carboxypeptidase II.

18. A compound of claim 4 in which the effector agent of ET is comprised of a group with the structure RN—L—V, wherein RN is a group that binds to the target biomolecule referred to as “rn”; and L is a linker, and V is a group that can covalently modify the target rn; and wherein RN—L—V can bind to rn and irreversibly chemically modify rn.

19. A compound of claim 18 in which V is comprised of a chemical group that generates free radicals and wherein the generated free radicals irreversibly chemically modify the target biomolecule rn.

20. A compound of claim 19 in which the free radical generator V is a non-radioactive metal-chelator complex.

21. A compound of claim 13 in which the effector agent is comprised of a structure that is modified by the enzymatic activity of a biomolecule and wherein this modification inactivates said biomolecule and in the process irreversibly chemically modifies said biomolecule.

22. A compound of claim 4 further comprising a second group wherein said second group binds to a receptor present in increased amounts at a target cell compared to at a non-target cell and wherein said second group is comprised of:
I. a monoclonal antibody; or
II. targeting receptor binding fragment of a monoclonal antibody; or
III. an analog or derivative which bears amino acid sequence similarity to portions of a monoclonal antibody; or
IV. a natural protein, or a complex of natural proteins, or a protein; or
V. a naturally occurring polymer.

23. A compound with a group, referred to as a “masked intracellular transport ligand” which can be modified in vivo to give a group referred to as an “intracellular transport ligand” which binds to a cell receptor that actively transports bound ligands into the cell.

24. A method of stimulating an immune response against a tumor and for treating a patient with cancer which comprises the following steps:
I. Immunizing or sensitizing a patient to a compound referred to as a neoantigen; and
II. Administering to the patient a compound referred to as a neoantigen generating compound; wherein said compound can irreversibly chemically modify a component of the tumor resulting in the generation of said neoantigen at the tumor.

25. A method of claim 24 in which the tumor component modified is selected from the following list:
1) Prostate specific Antigen
2) Human glandular kallikrein 2
3) Prostatic acid phosphatase
4) Plasmin
5) Placental type alkaline phosphatase
6) Matriptase
7) Matrix metalloproteinases
8) Thymidine phosphorylase
9) Trypsin
10) Urokinase
11) Fatty Acid Synthase
12) Steroid sulfatase
13) Epidermal growth factor receptor
14) Mitogen activated protein kinase kinase
15) Phosphatidylinositol 3-kinase
16) Mitogen activated protein kinase
17) Mitogen activated protein kinase
18) Thymidylate synthase
19) Protein kinase A
20) Fibroblast activation protein/ seprase
21) P-glycoprotein

26. A method of stimulating an immune response against a tumor and for treating a patient with cancer which comprises the following steps:
I. Immunizing or sensitizing a patient to a compound referred to as a neoantigen; and
II. Administering to the patient a compound of claim 13 referred to as a neoantigen generating compound; wherein said compound can irreversibly chemically modify a component of the tumor resulting in the generation of said neoantigen at the tumor.

27. A set of anticancer drugs referred to as “E1T1” and “E2T2” for use together or for co-administration to a patient, wherein E1 and E2 are effector agents that exhibit synergistic toxicity to a cell; and wherein T1 comprises a targeting ligand that binds to a first target receptor and T2 comprises a second targeting ligand that binds to the second target receptor which is increased on a tumor cell compared to a normal cell and where the first targeting ligand binds to a targeting receptor selected from the following list:
1) a cathepsin type protease
2) a collagenase
3) a gelatinase
4) a matrix metalloproteinase
5) a membrane type matrix metalloproteinase
6) alpha v beta 3 integrin
7) bombesin/gastrin releasing peptide receptors
8) cathepsin B
9) cathepsin D
10) cathepsin K
11) cathepsin L
12) cathepsin O
13) fibroblast activation protein
14) folate binding receptors
15) gastrin/cholecystokinin type B receptor
16) glutamate carboxypeptidase II or (PSMA)
17) guanidinobenzoatase
18) laminin receptor
19) matrilysin
20) matripase
21) melanocyte stimulating hormone receptor
22) nitrobenzylthioinosine-binding receptors
23) norepenephrine transporters
24) nucleoside transporter proteins
25) peripheral benzodiazepam binding receptors
26) plasmin
27) seprase
28) sigma receptors
29) somatostatin receptors
30) stromelysin 3
31) trypsin
32) urokinase
33) MMP 1
34) MMP 2
35) MMP 3
36) MMP 7
37) MMP 9
38) Membrane type matrix metalloproteinase I
39) MMP 12
40) MMP 13

28. A set of compounds of claim 27 wherein the effector agent E1 inhibits the denovo synthesis of a biomolecule(s) that is necessary for cell replication and/or survival, and the effector agent E2 inhibits a salvage pathway(s) that can enable a cell to by-pass the metabolic block caused by E1.

29. A set of compounds of E1T1 and E2T2 of claim 28 wherein T1 comprises a targeting ligand that binds to the first target receptor (a1); and T2 comprises a second targeting ligand that binds to the second target receptor (a2) indicated in the pairs of (a1—a2) listed below:
1) urokinase—a cathepsin type protease;
2) urokinase—a collagenase;
3) urokinase—a gelatinase;
4) urokinase—a matrix metalloproteinase;
5) urokinase—a membrane type matrix metalloproteinase;
6) urokinase—alpha v beta 3 integrin;
7) urokinase—bombesin/gastrin releasing peptide receptors;
8) urokinase—cathepsin B;
9) urokinase—cathepsin D;
10) urokinase—to cathepsin K;
11) urokinase—cathepsin L;
12) urokinase—cathepsin O;
13) urokinase—fibroblast activation protein;
14) urokinase—folate binding receptors;
15) urokinase—gastrin/cholecystokinin type B receptor;
16) urokinase—glutamate carboxypeptidase II or (PSMA);
17) urokinase guanidinobenzoatase;
18) urokinase—laminin receptor;
19) urokinase—matrilysin;
20) urokinase—matripase;
21) urokinase—melanocyte stimulating hormone receptor;
22) urokinase—nitrobenzylthioinosine-binding receptors or (nucleoside transporter);
23) urokinase—norepinephrine transporters;
24) urokinase—nucleoside transporter proteins;
25) urokinase—peripheral benzodiazepam binding receptors;
26) urokinase—plasmin;
27) urokinase—seprase;
28) urokinase—sigma receptors;
29) urokinase—somatostatin receptors;
30) urokinase—stromelysin 3;
31) urokinase—trypsin;
32) urokinase—urokinase;
33) urokinase—MMP 1;
34) urokinase—MMP 2;
35) urokinase—MMP 3;
36) urokinase—MMP 7;
37) urokinase—MMP 9;
38) urokinase—membrane type matrix metalloproteinase I;
39) urokinase—MMP 12;
40) urokinase—MMP 13;
41) urokinase—a tumor antigen;
42) plasmin—a cathepsin type protease;
43) plasmin—a collagenase;
44) plasmin—a gelatinase;
45) plasmin—a matrix metalloproteinase;
46) plasmin—a membrane type matrix metalloproteinase;
47) plasmin—alpha v beta 3 integrin;
48) plasmin—bombesin/gastrin releasing peptide receptors;
49) plasmin—cathepsin B;
50) plasmin—cathepsin D;
51) plasmin—to cathepsin K;
52) plasmin—cathepsin L;
53) plasmin—cathepsin O;
54) plasmin—fibroblast activation protein;
55) plasmin—folate binding receptors;
56) plasmin—gastrin/cholecystokinin type B receptor;
57) plasmin—glutamate carboxypeptidase II or (PSMA);
58) plasmin—guanidinobenzoatase;
59) plasmin—laminin receptor;
60) plasmin—matrilysin;
61) plasmin—matripase;
62) plasmin—melanocyte stimulating hormone receptor;
63) plasmin—nitrobenzylthioinosine-binding receptors or (nucleoside transporter);
64) plasmin—norepinephrine transporters;
65) plasmin—nucleoside transporter proteins;
66) plasmin—peripheral benzodiazepam binding receptors;
67) plasmin—plasmin;
68) plasmin—seprase;
69) plasmin—sigma receptors;
70) plasmin—somatostatin receptors;
71) plasmin—stromelysin 3;
72) plasmin—trypsin;
73) plasmin—urokinase;
74) plasmin—MMP 1;
75) plasmin—MMP 2;
76) plasmin—MMP 3;
77) plasmin—MMP 7;
78) plasmin—MMP 9;
79) plasmin—membrane type matrix metalloproteinase I;
80) plasmin—MMP 12;
81) plasmin—MMP 13;
82) plasmin—a tumor antigen;
83) a collagenase—a cathepsin type protease;
84) a collagenase—a collagenase;
85) a collagenase—a gelatinase;
86) a collagenase—a matrix metalloproteinase;
87) a collagenase—a membrane type matrix metalloproteinase;
88) a collagenase—alpha v beta 3 integrin;
89) a collagenase—bombesin/gastrin releasing peptide receptors;
90) a collagenase—cathepsin B;
91) a collagenase—cathepsin D;
92) a collagenase—to cathepsin K;
93) a collagenase—cathepsin L;
94) a collagenase—cathepsin O;
95) a collagenase—fibroblast activation protein;
96) a collagenase—folate binding receptors;
97) a collagenase—gastrin/cholecystokinin type B receptor;
98) a collagenase—glutamate carboxypeptidase II or (PSMA);
99) a collagenase—guanidinobenzoatase;
100) a collagenase—laminin receptor;
101) a collagenase—matrilysin;
102) a collagenase—matripase;
103) a collagenase—melanocyte stimulating hormone receptor;
104) a collagenase—nitrobenzylthioinosine-binding receptors or (nucleoside transporter);
105) a collagenase—norepinephrine transporters;
106) a collagenase—nucleoside transporter proteins;
107) a collagenase—peripheral benzodiazepam binding receptors;
108) a collagenase—seprase;
109) a collagenase—sigma receptors;
110) a collagenase—somatostatin receptors;
111) a collagenase—stromelysin 3;
112) a collagenase—trypsin;
113) a collagenase—a collagenase;
114) a collagenase—MMP 1;
115) a collagenase—MMP 2;
116) a collagenase—MMP 3;
117) a collagenase—MMP 7;
118) a collagenase—MMP 9;
119) a collagenase—membrane type matrix metalloproteinase I;
120) a collagenase—MMP 12;
121) a collagenase—MMP 13;
122) a collagenase—a tumor antigen;
123) a gelatinase—a cathepsin type protease;
124) a gelatinase—a gelatinase;
125) a gelatinase—a matrix metalloproteinase;
126) a gelatinase—a membrane type matrix metalloproteinase;
127) a gelatinase—alpha v beta 3 integrin;
128) a gelatinase—bombesin/gastrin releasing peptide receptors;
129) a gelatinase—cathepsin B;
130) a gelatinase—cathepsin D;
131) a gelatinase—to cathepsin K;
132) a gelatinase—cathepsin L;
133) a gelatinase—cathepsin O;
134) a gelatinase—fibroblast activation protein;
135) a gelatinase—folate binding receptors;
136) a gelatinase—gastrin/cholecystokinin type B receptor;
137) a gelatinase—glutamate carboxypeptidase II or (PSMA);
138) a gelatinase—guanidinobenzoatase;
139) a gelatinase—laminin receptor;
140) a gelatinase—matrilysin;
141) a gelatinase—matripase;
142) a gelatinase—melanocyte stimulating hormone receptor;
143) a gelatinase—nitrobenzylthioinosine-binding receptors or (nucleoside transporter);
144) a gelatinase—norepinephrine transporters;
145) a gelatinase—nucleoside transporter proteins;
146) a gelatinase—peripheral benzodiazepam binding receptors;
147) a gelatinase—seprase;
148) a gelatinase—sigma receptors;
149) a gelatinase—somatostatin receptors;
150) a gelatinase—stromelysin 3;
151) a gelatinase—trypsin;
152) a gelatinase—MMP 1;
153) a gelatinase—MMP 2;
154) a gelatinase—MMP 3;
155) a gelatinase—MMP 7;
156) a gelatinase—MMP 9;
157) a gelatinase—membrane type matrix metalloproteinase I;
158) a gelatinase—MMP 12;
159) a gelatinase—MMP 13;
160) a gelatinase—a tumor antigen;
161) a matrix metalloproteinase—a cathepsin type protease;
162) a matrix metalloproteinase—a collagenase;
163) a matrix metalloproteinase—a gelatinase;
164) a matrix metalloproteinase—a matrix metalloproteinase;
165) a matrix metalloproteinase—a membrane type matrix metalloproteinase;
166) a matrix metalloproteinase—alpha v beta 3 integrin;
167) a matrix metalloproteinase—bombesin/gastrin releasing peptide receptors;
168) a matrix metalloproteinase—cathepsin B;
169) a matrix metalloproteinase—cathepsin D;
170) a matrix metalloproteinase—to cathepsin K;
171) a matrix metalloproteinase—cathepsin L;
172) a matrix metalloproteinase—cathepsin O;
173) a matrix metalloproteinase—fibroblast activation protein;
174) a matrix metalloproteinase—folate binding receptors;
175) a matrix metalloproteinase—gastrin/cholecystokinin type B receptor;
176) a matrix metalloproteinase—glutamate carboxypeptidase II or (PSMA);
177) a matrix metalloproteinase—guanidinobenzoatase;
178) a matrix metalloproteinase—laminin receptor;
179) a matrix metalloproteinase—matrilysin;
180) a matrix metalloproteinase—matripase;
181) a matrix metalloproteinase—melanocyte stimulating hormone receptor;
182) a matrix metalloproteinase—nitrobenzylthioinosine-binding receptors or (nucleoside transporter);
183) a matrix metalloproteinase—norepinephrine transporters;
184) a matrix metalloproteinase—nucleoside transporter proteins;
185) a matrix metalloproteinase—peripheral benzodiazepam binding receptors;
186) a matrix metalloproteinase—plasmin;
187) a matrix metalloproteinase—seprase;
188) a matrix metalloproteinase—sigma receptors;
189) a matrix metalloproteinase—somatostatin receptors;
190) a matrix metalloproteinase—stromelysin 3;
191) a matrix metalloproteinase—trypsin;
192) a matrix metalloproteinase—a matrix metalloproteinase;
193) a matrix metalloproteinase—MMP 1;
194) a matrix metalloproteinase—MMP 2;
195) a matrix metalloproteinase—MMP 3;
196) a matrix metalloproteinase—MMP 7;
197) a matrix metalloproteinase—MMP 9;
198) a matrix metalloproteinase—membrane type matrix metalloproteinase I;
199) a matrix metalloproteinase—MMP 12;
200) a matrix metalloproteinase—MMP 13;
201) a matrix metalloproteinase—a tumor antigen;
202) a membrane type metalloproteinase—a cathepsin type protease;
203) a membrane type metalloproteinase—a membrane type matrix metalloproteinase;
204) a membrane type metalloproteinase—alpha v beta 3 integrin;
205) a membrane type metalloproteinase—bombesin/gastrin releasing peptide receptors;
206) a membrane type metalloproteinase—cathepsin B;
207) a membrane type metalloproteinase—cathepsin D;
208) a membrane type metalloproteinase—to cathepsin K;
209) a membrane type metalloproteinase—cathepsin L;
210) a membrane type metalloproteinase—cathepsin O;
211) a membrane type metalloproteinase—fibroblast activation protein;
212) a membrane type metalloproteinase—folate binding receptors;
213) a membrane type metalloproteinase—gastrin/cholecystokinin type B receptor;
214) a membrane type metalloproteinase—glutamate carboxypeptidase II or (PSMA);
215) a membrane type metalloproteinase—guanidinobenzoatase;
216) a membrane type metalloproteinase—laminin receptor;
217) a membrane type metalloproteinase matrilysin;
218) a membrane type metalloproteinase—matripase;
219) a membrane type metalloproteinase—melanocyte stimulating hormone receptor;
220) a membrane type metalloproteinase—nitrobenzylthioinosine-binding receptors or (nucleoside transporter);
221) a membrane type metalloproteinase—norepinephrine transporters;
222) a membrane type metalloproteinase—nucleoside transporter proteins;
223) a membrane type metalloproteinase—peripheral benzodiazepam binding receptors;
224) a membrane type metalloproteinase—seprase;
225) a membrane type metalloproteinase—sigma receptors;
226) a membrane type metalloproteinase—somatostatin receptors;
227) a membrane type metalloproteinase—stromelysin 3;
228) a membrane type metalloproteinase—trypsin;
229) a membrane type metalloproteinase—MMP 1;
230) a membrane type metalloproteinase—MMP 2;
231) a membrane type metalloproteinase—MMP 3;
232) a membrane type metalloproteinase—MMP 7;
233) a membrane type metalloproteinase—MMP 9;
234) a membrane type metalloproteinase—membrane type matrix metalloproteinase I;
235) a membrane type metalloproteinase—MMP 12;
236) a membrane type metalloproteinase—MMP 13;
237) a membrane type metalloproteinase—a tumor antigen;
238) alpha v beta 3 integrin—a cathepsin type protease;
239) alpha v beta 3 integrin—alpha v beta 3 integrin;
240) alpha v beta 3 integrin—bombesin/gastrin releasing peptide receptors;
241) alpha v beta 3 integrin—cathepsin B;
242) alpha v beta 3 integrin—cathepsin D;
243) alpha v beta 3 integrin—cathepsin K;
244) alpha v beta 3 integrin—cathepsin L;
245) alpha v beta 3 integrin—cathepsin O;
246) alpha v beta 3 integrin—fibroblast activation protein;
247) alpha v beta 3 integrin—folate binding receptors;
248) alpha v beta 3 integrin—gastrin/cholecystokinin type B receptor;
249) alpha v beta 3 integrin—glutamate carboxypeptidase II or (PSMA);
250) alpha v beta 3 integrin—guanidinobenzoatase;
251) alpha v beta 3 integrin—laminin receptor;
252) alpha v beta 3 integrin—matrilysin;
253) alpha v beta 3 integrin—matripase;
254) alpha v beta 3 integrin—melanocyte stimulating hormone receptor;
255) alpha v beta 3 integrin—nitrobenzylthioinosine-binding receptors or (nucleoside transporter);
256) alpha v beta 3 integrin—norepinephrine transporters;
257) alpha v beta 3 integrin—nucleoside transporter proteins;
258) alpha v beta 3 integrin—peripheral benzodiazepam binding receptors;
259) alpha v beta 3 integrin—seprase;
260) alpha v beta 3 integrin—sigma receptors;
261) alpha v beta 3 integrin—somatostatin receptors;
262) alpha v beta 3 integrin—stromelysin 3;
263) alpha v beta 3 integrin—trypsin;
264) alpha v beta 3 integrin—MMP 1;
265) alpha v beta 3 integrin—MMP 2;
266) alpha v beta 3 integrin—MMP 3;
267) alpha v beta 3 integrin—MMP 7;
268) alpha v beta 3 integrin—MMP 9;
269) alpha v beta 3 integrin—membrane type matrix metalloproteinase I;
270) alpha v beta 3 integrin—MMP 12;
271) alpha v beta 3 integrin—MMP 13;
272) alpha v beta 3 integrin—a tumor antigen;
273) cathepsin B—a cathepsin type protease;
274) cathepsin B—bombesin/gastrin releasing peptide receptors;
275) cathepsin B—cathepsin B;
276) cathepsin B—cathepsin D;
277) cathepsin B—to cathepsin K;
278) cathepsin B—cathepsin L;
279) cathepsin B—cathepsin O;
280) cathepsin B—fibroblast activation protein;
281) cathepsin B—folate binding receptors;
282) cathepsin B—gastrin/cholecystokinin type B receptor;
283) cathepsin B—glutamate carboxypeptidase II or (PSMA);
284) cathepsin B—guanidinobenzoatase;
285) cathepsin B—laminin receptor;
286) cathepsin B—matrilysin;
287) cathepsin B—matripase;
288) cathepsin B—melanocyte stimulating hormone receptor;
289) cathepsin B—nitrobenzylthioinosine-binding receptors or (nucleoside transporter);
290) cathepsin B—norepinephrine transporters;
291) cathepsin B—nucleoside transporter proteins;
292) cathepsin B—peripheral benzodiazepam binding receptors;
293) cathepsin B—seprase;
294) cathepsin B—sigma receptors;
295) cathepsin B—somatostatin receptors;
296) cathepsin B—stromelysin 3;
297) cathepsin B—trypsin;
298) cathepsin B—MMP 1;
299) cathepsin B—MMP 2;
300) cathepsin B—MMP 3;
301) cathepsin B—MMP 7;
302) cathepsin B—MMP 9;
303) cathepsin B—membrane type matrix metalloproteinase I;
304) cathepsin B—MMP 12;
305) cathepsin B—MMP 13;
306) cathepsin B—a tumor antigen;
307) bombesin/gastrin releasing peptide receptors—a cathepsin type protease;
308) bombesin/gastrin releasing peptide receptors—bombesin/gastrin releasing peptide receptors;
309) bombesin/gastrin releasing peptide receptors—cathepsin B;
310) bombesin/gastrin releasing peptide receptors—cathepsin D;
311) bombesin/gastrin releasing peptide receptors—to cathepsin K;
312) bombesin/gastrin releasing peptide receptors—cathepsin L;
313) bombesin/gastrin releasing peptide receptors—cathepsin O;
314) bombesin/gastrin releasing peptide receptors—fibroblast activation protein;
315) bombesin/gastrin releasing peptide receptors—folate binding receptors;
316) bombesin/gastrin releasing peptide receptors—gastrin/cholecystokinin type B receptor;
317) bombesin/gastrin releasing peptide receptors—glutamate carboxypeptidase II or (PSMA);
318) bombesin/gastrin releasing peptide receptors—guanidinobenzoatase;
319) bombesin/gastrin releasing peptide receptors—laminin receptor;
320) bombesin/gastrin releasing peptide receptors—matrilysin;
321) bombesin/gastrin releasing peptide receptors matripase;
322) bombesin/gastrin releasing peptide receptors—melanocyte stimulating hormone receptor;
323) bombesin/gastrin releasing peptide receptors—nitrobenzylthioinosine-binding receptor s or (nucleoside transporter);
324) bombesin/gastrin releasing peptide receptors—norepinephrine transporters;
325) bombesin/gastrin releasing peptide receptors—nucleoside transporter proteins;
326) bombesin/gastrin releasing peptide receptors—peripheral benzodiazepam binding receptors;
327) bombesin/gastrin releasing peptide receptors—seprase;
328) bombesin/gastrin releasing peptide receptors—sigma receptors;
329) bombesin/gastrin releasing peptide receptors—somatostatin receptors;
330) bombesin/gastrin releasing peptide receptors—stromelysin 3;
331) bombesin/gastrin releasing peptide receptors—trypsin;
332) bombesin/gastrin releasing peptide receptors—MMP 1;
333) bombesin/gastrin releasing peptide receptors—MMP 2;
334) bombesin/gastrin releasing peptide receptors—MMP 3;
335) bombesin/gastrin releasing peptide receptors—MMP 7;
336) bombesin/gastrin releasing peptide receptors—MMP 9;
337) bombesin/gastrin releasing peptide receptors—membrane type matrix metalloproteinase I;
338) bombesin/gastrin releasing peptide receptors MMP 12;
339) bombesin/gastrin releasing peptide receptors—MMP 13;
340) bombesin/gastrin releasing peptide receptors—a tumor antigen;
341) fibroblast activation protein—a cathepsin type protease;
342) fibroblast activation protein—cathepsin D;
343) fibroblast activation protein—to cathepsin K;
344) fibroblast activation protein—cathepsin L;
345) fibroblast activation protein—cathepsin O;
346) fibroblast activation protein—fibroblast activation protein;
347) fibroblast activation protein—folate binding receptors;
348) fibroblast activation protein—gastrin/cholecystokinin type B receptor;
349) fibroblast activation protein—glutamate carboxypeptidase II or (PSMA);
350) fibroblast activation protein—guanidinobenzoatase;
351) fibroblast activation protein—laminin receptor;
352) fibroblast activation protein—matrilysin;
353) fibroblast activation protein—matripase;
354) fibroblast activation protein—melanocyte stimulating hormone receptor;
355) fibroblast activation protein—nitrobenzylthioinosine-binding receptors or (nucleoside transporter);
356) fibroblast activation protein—norepinephrine transporters;
357) fibroblast activation protein—nucleoside transporter proteins;
358) fibroblast activation protein—peripheral benzodiazepam binding receptors;
359) fibroblast activation protein—plasmin;
360) fibroblast activation protein—seprase;
361) fibroblast activation protein—sigma receptors;
362) fibroblast activation protein—somatostatin receptors;
363) fibroblast activation protein—stromelysin 3;
364) fibroblast activation protein—trypsin;
365) fibroblast activation protein—MMP 1;
366) fibroblast activation protein—MMP 2;
367) fibroblast activation protein—MMP 3;
368) fibroblast activation protein—MMP 7;
369) fibroblast activation protein—MMP 9;
370) fibroblast activation protein—membrane type matrix metalloproteinase I;
371) fibroblast activation protein—MMP 12;
372) fibroblast activation protein—MMP 13;
373) fibroblast activation protein—a tumor antigen;
374) glutamate carboxypeptidase II or PSMA—cathepsin D;
375) glutamate carboxypeptidase II or PSMA—to cathepsin K;
376) glutamate carboxypeptidase II or PSMA—cathepsin L;
377) glutamate carboxypeptidase II or PSMA—cathepsin O;
378) glutamate carboxypeptidase II or PSMA—fibroblast activation protein;
379) glutamate carboxypeptidase II or PSMA—folate binding receptors;
380) glutamate carboxypeptidase II or PSMA—gastrin/cholecystokinin type B receptor;
381) glutamate carboxypeptidase II or PSMA—glutamate carboxypeptidase II or (PSMA);
382) glutamate carboxypeptidase II or PSMA—guanidinobenzoatase;
383) glutamate carboxypeptidase II or PSMA—laminin receptor;
384) glutamate carboxypeptidase II or PSMA—matrilysin;
385) glutamate carboxypeptidase II or PSMA—matripase;
386) glutamate carboxypeptidase II or PSMA—melanocyte stimulating hormone receptor;
387) glutamate carboxypeptidase II or PSMA—nitrobenzylthioinosine-binding receptors or (nucleoside transporter);
388) glutamate carboxypeptidase II or PSMA—nucleoside transporter proteins;
389) glutamate carboxypeptidase II or PSMA—peripheral benzodiazepam binding receptors;
390) glutamate carboxypeptidase II or PSMA—seprase;
391) glutamate carboxypeptidase II or PSMA—sigma receptors;
392) glutamate carboxypeptidase II or PSMA—somatostatin receptors;
393) glutamate carboxypeptidase II or PSMA—stromelysin 3;
394) glutamate carboxypeptidase II or PSMA—trypsin;
395) glutamate carboxypeptidase II or PSMA—MMP 1;
396) glutamate carboxypeptidase II or PSMA—MMP 2;
397) glutamate carboxypeptidase II or PSMA—MMP 3;
398) glutamate carboxypeptidase II or PSMA—MMP 7;
399) glutamate carboxypeptidase II or PSMA—MMP 9;
400) glutamate carboxypeptidase II or PSMA—membrane type matrix metalloproteinase I;
401) glutamate carboxypeptidase II or PSMA—MMP 12;
402) glutamate carboxypeptidase II or PSMA—MMP 13;
403) glutamate carboxypeptidase II or PSMA—a tumor antigen;
404) laminin receptor—a cathepsin type protease;
405) laminin receptor—cathepsin B;
406) laminin receptor—cathepsin D;
407) laminin receptor—to cathepsin K;
408) laminin receptor—cathepsin L;
409) laminin receptor—cathepsin O;
410) laminin receptor—fibroblast activation protein;
411) laminin receptor—folate binding receptors;
412) laminin receptor—gastrin/cholecystokinin type B receptor;
413) laminin receptor—guanidinobenzoatase;
414) laminin receptor—laminin receptor;
415) laminin receptor—matrilysin;
416) laminin receptor—matripase;
417) laminin receptor—melanocyte stimulating hormone receptor;
418) laminin receptor—nitrobenzylthioinosine-binding receptors or (nucleoside transporter);
419) laminin receptor—norepinephrine transporters;
420) laminin receptor—nucleoside transporter proteins;
421) laminin receptor—peripheral benzodiazepam binding receptors;
422) laminin receptor—seprase;
423) laminin receptor—sigma receptors;
424) laminin receptor—somatostatin receptors;
425) laminin receptor—stromelysin 3;
426) laminin receptor—trypsin;
427) laminin receptor—MMP 1;
428) laminin receptor—MMP 2;
429) laminin receptor—MMP 3;
430) laminin receptor—MMP 7;
431) laminin receptor—MMP 9;
432) laminin receptor—membrane type matrix metalloproteinase I;
433) laminin receptor—MMP 12;
434) laminin receptor—MMP 13;
435) laminin receptor—a tumor antigen;
436) seprase—a cathepsin type protease;
437) seprase—cathepsin D;
438) seprase—to cathepsin K;
439) seprase—cathepsin L;
440) seprase—cathepsin O;
441) seprase—fibroblast activation protein;
442) seprase—folate binding receptors;
443) seprase—gastrin/cholecystokinin type B receptor;
444) seprase—guanidinobenzoatase;
445) seprase—matripase;
446) seprase—melanocyte stimulating hormone receptor;
447) seprase—nitrobenzylthioinosine-binding receptors or (nucleoside transporter);
448) seprase—norepinephrine transporters;
449) seprase—nucleoside transporter proteins;
450) seprase—peripheral benzodiazepam binding receptors;
451) seprase—seprase;
452) seprase—sigma receptors;
453) seprase—somatostatin receptors;
454) seprase—stromelysin 3;
455) seprase—trypsin;
456) seprase—MMP 1;
457) seprase—MMP 2;
458) seprase—MMP 3;
459) seprase—MMP 7;
460) seprase—MMP 9;
461) seprase—membrane type matrix metalloproteinase I;
462) seprase—MMP 12;
463) seprase—MMP 13;
464) seprase—a tumor antigen;
465) guanidinobenzoatase—a cathepsin type protease;
466) guanidinobenzoatase—cathepsin D;
467) guanidinobenzoatase—to cathepsin K;
468) guanidinobenzoatase—cathepsin L;
469) guanidinobenzoatase—cathepsin O;
470) guanidinobenzoatase—fibroblast activation protein;
471) guanidinobenzoatase—folate binding receptors;
472) guanidinobenzoatase—gastrin/cholecystokinin type B receptor;
473) guanidinobenzoatase—guanidinobenzoatase;
474) guanidinobenzoatase—matripase;
475) guanidinobenzoatase—melanocyte stimulating hormone receptor;
476) guanidinobenzoatase—nitrobenzylthioinosine-binding receptors or (nucleoside transporter);
477) guanidinobenzoatase—norepinephrine transporters;
478) guanidinobenzoatase—nucleoside transporter proteins;
479) guanidinobenzoatase—peripheral benzodiazepam binding receptors;
480) guanidinobenzoatase—sigma receptors;
481) guanidinobenzoatase—somatostatin receptors;
482) guanidinobenzoatase—stromelysin 3;
483) guanidinobenzoatase—trypsin;
484) guanidinobenzoatase—MMP 1;
485) guanidinobenzoatase—MMP 2;
486) guanidinobenzoatase—MMP3 487) guanidinobenzoatase—MMP 7;
488) guanidinobenzoatase—MMP 9;
489) guanidinobenzoatase—membrane type matrix metalloproteinase
490) guanidinobenzoatase—MMP 12;
491) guanidinobenzoatase—MMP 13;
492) guanidinobenzoatase—a tumor antigen;
493) peripheral benzodiazepam binding receptors—a cathepsin type protease;
494) peripheral benzodiazepam binding receptors—cathepsin D;
495) peripheral benzodiazepam binding receptors—to cathepsin K;
496) peripheral benzodiazepam binding receptors—cathepsin L;
497) peripheral benzodiazepam binding receptors—cathepsin O;
498) peripheral benzodiazepam binding receptors—fibroblast activation protein;
499) peripheral benzodiazepam binding receptors—folate binding receptors;
500) peripheral benzodiazepam binding receptors—gastrin/cholecystokinin type B receptor;
501) peripheral benzodiazepam binding receptors—guanidinobenzoatase;
502) peripheral benzodiazepam binding receptors—matripase;
503) peripheral benzodiazepam binding receptors—melanocyte stimulating hormone receptor;
504) peripheral benzodiazepam binding receptors—nitrobenzylthioinosine-binding receptors or (nucleoside transporter);
505) peripheral benzodiazepam binding receptors—norepinephrine transporters;
506) peripheral benzodiazepam binding receptors—nucleoside transporter proteins;
507) peripheral benzodiazepam binding receptors—peripheral benzodiazepam binding receptors;
508) peripheral benzodiazepam binding receptors—sigma receptors;
509) peripheral benzodiazepam binding receptors—somatostatin receptors;
510) peripheral benzodiazepam binding receptors—stromelysin 3;
511) peripheral benzodiazepam binding receptors—trypsin;
512) peripheral benzodiazepam binding receptors—MMP 1;
513) peripheral benzodiazepam binding receptors—MMP 2;
514) peripheral benzodiazepam binding receptors—MMP 3;
515) peripheral benzodiazepam binding receptors—MMP 7;
516) peripheral benzodiazepam binding receptors—MMP 9;
517) peripheral benzodiazepam binding receptors—membrane type matrix metalloproteinase I;
518) peripheral benzodiazepam binding receptors—MMP 12;
519) peripheral benzodiazepam binding receptors—MMP 13;
520) peripheral benzodiazepam binding receptors—a tumor antigen;
521) folate binding receptors—a cathepsin type protease;
522) folate binding receptors—cathepsin D;
523) folate binding receptors—to cathepsin K;
524) folate binding receptors cathepsin L;
525) folate binding receptors—cathepsin O;
526) folate binding receptors—fibroblast activation protein;
527) folate binding receptors—folate binding receptors;
528) folate binding receptors—matripase;
529) folate binding receptors—melanocyte stimulating hormone receptor;
530) folate binding receptors—nitrobenzylthioinosine-binding receptors or (nucleoside transporter);
531) folate binding receptors—norepinephrine transporters;
532) folate binding receptors—nucleoside transporter proteins;
533) folate binding receptors—sigma receptors;
534) folate binding receptors—somatostatin receptors;
535) folate binding receptors—stromelysin 3;
536) folate binding receptors—trypsin;
537) folate binding receptors—MMP 1;
538) folate binding receptors—MMP 2;
539) folate binding receptors—MMP 3;
540) folate binding receptors—MMP 7;
541) folate binding receptors—MMP 9;
542) folate binding receptors—membrane type matrix metalloproteinase I;
543) folate binding receptors—MMP 12;
544) folate binding receptors—MMP 13;
545) folate binding receptors—a tumor antigen;
546) folate binding receptors—a cathepsin type protease;
547) folate binding receptors—cathepsin D;
548) folate binding receptors—to cathepsin K;
549) folate binding receptors—cathepsin L;
550) folate binding receptors—cathepsin O;
551) folate binding receptors—fibroblast activation protein;
552) folate binding receptors—folate binding receptors;
553) folate binding receptors—matripase;
554) folate binding receptors—melanocyte stimulating hormone receptor;
555) folate binding receptors—nitrobenzylthioinosine-binding receptors or (nucleoside transporter);
556) folate binding receptors—norepinephrine transporters;
557) folate binding receptors—nucleoside transporter proteins;
558) folate binding receptors—sigma receptors;
559) folate binding receptors—somatostatin receptors;
560) folate binding receptors—stromelysin 3;
561) folate binding receptors—trypsin;
562) folate binding receptors—MMP 1;
563) folate binding receptors—MMP 2;
564) folate binding receptors—MMP 3;
565) folate binding receptors—MMP 7;
566) folate binding receptors—MMP 9;
567) folate binding receptors—membrane type matrix metalloproteinase I;
568) folate binding receptors—MMP 12;
569) folate binding receptors—MMP 13;
570) folate binding receptors—a tumor antigen;
571) nucleoside transporter proteins—a cathepsin type protease;
572) nucleoside transporter proteins cathepsin D;
573) nucleoside transporter proteins—to cathepsin K;
574) nucleoside transporter proteins—cathepsin L;
575) nucleoside transporter proteins—cathepsin O;
576) nucleoside transporter proteins—fibroblast activation protein;
577) nucleoside transporter proteins—nucleoside transporter proteins;
578) nucleoside transporter proteins—matripase;
579) nucleoside transporter proteins—melanocyte stimulating hormone receptor;
580) nucleoside transporter proteins—nitrobenzylthioinosine-binding receptors or (nucleoside transporter);
581) nucleoside transporter proteins—norepinephrine transporters;
582) nucleoside transporter proteins—nucleoside transporter proteins;
583) nucleoside transporter proteins—sigma receptors;
584) nucleoside transporter proteins—somatostatin receptors;
585) nucleoside transporter proteins—stromelysin 3;
586) nucleoside transporter proteins—trypsin;
587) nucleoside transporter proteins—MMP 1;
588) nucleoside transporter proteins—MMP 2;
589) nucleoside transporter proteins—MMP 3;
590) nucleoside transporter proteins—MMP 7;
591) nucleoside transporter proteins—MMP 9;
592) nucleoside transporter proteins—membrane type matrix metalloproteinase I;
593) nucleoside transporter proteins—MMP 12;
594) nucleoside transporter proteins—MMP 13;
595) nucleoside transporter proteins—a tumor antigen;
596) melanocyte stimulating hormone receptor—a cathepsin type protease;
597) melanocyte stimulating hormone receptor—cathepsin D;
598) melanocyte stimulating hormone receptor—to cathepsin K;
599) melanocyte stimulating hormone receptor—cathepsin L;
600) melanocyte stimulating hormone receptor—cathepsin O;
601) melanocyte stimulating hormone receptor—fibroblast activation protein;
602) melanocyte stimulating hormone receptor—melanocyte stimulating hormone receptor;
603) melanocyte stimulating hormone receptor—melanocyte stimulating hormone receptor;
604) melanocyte stimulating hormone receptor—nitrobenzylthioinosine-binding receptors or (nucleoside transporter);
605) melanocyte stimulating hormone receptor—norepinephrine transporters;
606) melanocyte stimulating hormone receptor—nucleoside transporter
proteins;
607) melanocyte stimulating hormone receptor—sigma receptors;
608) melanocyte stimulating hormone receptor—somatostatin receptors;
609) melanocyte stimulating hormone receptor—stromelysin 3;
610) melanocyte stimulating hormone receptor—trypsin;
611) melanocyte stimulating hormone receptor—MMP 1;
612) melanocyte stimulating hormone receptor—MMP 2;
613) melanocyte stimulating hormone receptor—MMP 3;
614) melanocyte stimulating hormone receptor—MMP 7;
615) melanocyte stimulating hormone receptor—MMP 9;
616) melanocyte stimulating hormone receptor—membrane type matrix metalloproteinase I;
617) melanocyte stimulating hormone receptor—MMP 12;
618) melanocyte stimulating hormone receptor—MMP 13;
619) melanocyte stimulating hormone receptor—a tumor antigen;
620) sigma receptors—a cathepsin type protease;
621) sigma receptors—cathepsin D;
622) sigma receptors—to cathepsin K;
623) sigma receptors—cathepsin L;
624) sigma receptors—cathepsin O;
625) sigma receptors—fibroblast activation protein;
626) sigma receptors—sigma receptors;
627) sigma receptors—matripase;
628) sigma receptors—norepinephrine transporters;
629) sigma receptors—sigma receptors;
630) sigma receptors—somatostatin receptors;
631) sigma receptors—stromelysin 3;
632) sigma receptors—trypsin;
633) sigma receptors—MMP 1;
634) sigma receptors—MMP 2;
635) sigma receptors—MMP 3;
636) sigma receptors—MMP 7;
637) sigma receptors—MMP 9;
638) sigma receptors—membrane type matrix metalloproteinase I;
639) sigma receptors—MMP 12;
640) sigma receptors—MMP 13;
641) sigma receptors—a tumor antigen;
642) somatostatin receptors—a cathepsin type protease;
643) somatostatin receptors—cathepsin D;
644) somatostatin receptors—to cathepsin K;
645) somatostatin receptors—cathepsin L;
646) somatostatin receptors—cathepsin O;
647) somatostatin receptors—fibroblast activation protein;
648) somatostatin receptors—somatostatin receptors;
649) somatostatin receptors—matripase;
650) somatostatin receptors—melanocyte stimulating hormone receptor;
651) somatostatin receptors—sigma receptors;
652) somatostatin receptors—somatostatin receptors;
653) somatostatin receptors—stromelysin 3;
654) somatostatin receptors—trypsin;
655) somatostatin receptors—MMP 1;
656) somatostatin receptors—MMP 2;
657) somatostatin receptors—MMP 3;
658) somatostatin receptors—MMP 7;
659) somatostatin receptors—MMP 9;
660) somatostatin receptors—membrane type matrix metalloproteinase I;
661) somatostatin receptors—MMP 12;
662) somatostatin receptors—MMP 13;
663) somatostatin receptors—a tumor antigen
664) stromelysin 3—a cathepsin type protease;
665) stromelysin 3—cathepsin D;
666) stromelysin 3—to cathepsin K;
667) stromelysin 3—cathepsin L;
668) stromelysin 3—cathepsin O;
669) stromelysin 3—fibroblast activation protein;
670) stromelysin 3—stromelysin 3;
671) stromelysin 3—matripase;
672) stromelysin 3—melanocyte stimulating hormone receptor;
673) stromelysin 3—somatostatin receptors;
674) stromelysin 3—trypsin;
675) stromelysin 3—MMP 1;
676) stromelysin 3—MMP 2;
677) stromelysin 3—MMP 3;
678) stromelysin 3—MMP 7;
679) stromelysin 3—MMP 9;
680) stromelysin 3—membrane type matrix metalloproteinase I;
681) stromelysin 3—MMP 2;
682) stromelysin 3—MMP 13;
683) stromelysin 3—a tumor antigen;
684) trypsin—a cathepsin type protease;
685) trypsin—cathepsin D;
686) trypsin—to cathepsin K;
687) trypsin—cathepsin L;
688) trypsin—cathepsin O;
689) trypsin—fibroblast activation protein;
690) trypsin—trypsin;
691) trypsin—matripase;
692) trypsin—melanocyte stimulating hormone receptor;
693) trypsin—stromelysin 3;
694) trypsin—MMP 1;
695) trypsin—MMP 2;
696) trypsin—MMP 3;
697) trypsin—MMP 7;
698) trypsin—MMP 9;
699) trypsin—membrane type matrix metalloproteinase I;
700) trypsin—MMP 12;
701) trypsin—MMP 13;
702) trypsin—a tumor antigen;
703) MMP 1—a cathepsin type protease;
704) MMP 1—cathepsin D;
705) MMP 1—to cathepsin K;
706) MMP 1—cathepsin L;
707) MMP 1—cathepsin O;
708) MMP 1—fibroblast activation protein;
709) MMP 1—matripase;
710) MMP 1—melanocyte stimulating hormone receptor;
711) MMP 1—stromelysin 3;
712) MMP 1—MMP 1;
713) MMP 1—MMP 2;
714) MMP 1—MMP 3;
715) MMP 1—MMP 7;
716) MMP 1—MMP 9;
717) MMP 1—membrane type matrix metalloproteinase I;
718) MMP1—MMP12;
719) MMP1—MMP13;
720) MMP 1—a tumor antigen;
721) MMP-2—a cathepsin type protease;
722) MMP-2—cathepsin D;
723) MMP-2—to cathepsin K;
724) MMP-2—cathepsin L;
725) MMP-2—cathepsin O;
726) MMP-2—fibroblast activation protein;
727) MMP-2—matripase;
728) MMP-2—melanocyte stimulating hormone receptor;
729) MMP-2—stromelysin 3;
730) MMP-2—MMP 2;
731) MMP-2—MMP3;
732) MMP-2—MMP 7;
733) MMP-2—MMP 9;
734) MMP-2—membrane type matrix metalloproteinase I;
735) MMP-2—MMP-2;
736) MMP-2—MMP-3;
737) MMP-2—a tumor antigen;
738) MMP-3—a cathepsin type protease;
739) MMP-3—cathepsin D;
740) MMP-3—to cathepsin K;
741) MMP-3—cathepsin L;
742) MMP-3—cathepsin O;
743) MMP-3—matripase;
744) MMP-3—MMP 3;
745) MMP-3—MMP 7;
746) MMP-3—MMP 9;
747) MMP-3—membrane type matrix metalloproteinase I;
748) MMP-3—MMP-3;
749) MMP-3—a tumor antigen;
750) MMP 7—a cathepsin type protease;
751) MMP7—cathepsin D;
752) MMP 7—to cathepsin K;
753) MMP 7—cathepsin L;
754) MMP 7—cathepsin I;
755) MMP 7—fibroblast activation protein;
756) MMP 7—matripase;
757) MMP 7—stromelysin 3;
758) MMP 7—MMP 7;
759) MMP 7—MMP 9;
760) MMP 7—membrane type matrix metalloproteinase I;
761) MMP 7—a tumor antigen;
762) MMP 9—a cathepsin type protease;
763) MMP 9—cathepsin D;
764) MMP 9—to cathepsin K;
765) MMP 9—cathepsin L;
766) MMP 9—cathepsin O;
767) MMP 9—matripase;
768) MMP 9—MMP 9;
769) MMP 9—membrane type matrix metalloproteinase I;
770) MMP 9—a tumor antigen;
771) MMP 12—a cathepsin type protease;
772) MMP 12—cathepsin D;
773) MMP 12—to cathepsin K;
774) MMP 12—cathepsin L;
775) MMP12—cathepsin L;
776) MMP 12—matripase;
777) MMP12—MMP2;
778) MMP 12—membrane type matrix metalloproteinase I;
779) MMP 12—a tumor antigen;
780) MMP 13—a cathepsin type protease;
781) MMP13—cathepsin D;
782) MMP 13—to cathepsin K;
783) MMP 13—cathepsin L;
784) MMP 13—cathepsin O;
785) MMP 13—matripase;
786) MMP 13—membrane type matrix metalloproteinase I;
787) MMP 13—a tumor antigen;
788) Membrane type matrix metalloproteinase—a cathepsin type protease;
789) Membrane type matrix metalloproteinase—cathepsin D;
790) Membrane type matrix metalloproteinase—to cathepsin K;
791) Membrane type matrix metalloproteinase—cathepsin L;
792) Membrane type matrix metalloproteinase—cathepsin O;
793) Membrane type matrix metalloproteinase—matripase;
794) Membrane type matrix metalloproteinase—membrane type matrix metalloproteinase I; and
795) Membrane type matrix metalloproteinase—a tumor antigen.
